(19) United States
(12) Patent Application Publication
(10) Pub. No.: US 2024/0268980 A1
(43) Pub. Date: Aug. 15, 2024

(54) STRETCHING DEVICE

(71) Applicant: Peter Forsell, Bouveret (CH)

(72) Inventor: Peter Forsell, Bouveret (CH)

(21) Appl. No.: 18/443,387

(22) Filed: Feb. 16, 2024

Related U.S. Application Data
(63) Continuation-in-part of application No. PCT/EP2022/073784, filed on Aug. 26, 2022.
(63) Continuation-in-part of application No. PCT/EP2023/053903, filed on Feb. 16, 2023.

(30) Foreign Application Priority Data
Aug. 30, 2021 (WO) PCT/EP2021/073893
Feb. 18, 2022 (SE) 2250181-1
Feb. 18, 2022 (SE) 2250203-3
Aug. 26, 2022 (WO) PCT/EP2022/073784

(51) Int. Cl. A61F 5/00

(52) U.S. Cl. CPC A61F 5/0069; A61F 2005/002; A61F 2005/0023; A61F 5/0026

(57) ABSTRACT

A medical device for actively stretching a stomach wall of a patient for creating a sensation of satiety. The medical device being implantable and comprising a first member configured to be fixated to a first portion of the stomach wall, a second member configured to be fixated to a second portion of the stomach wall, an operation device for operating the second member to displace the second member in a first direction relative to the first member, for stretching the stomach wall between the first and second portion.

This application is a continuation in part of International Application No. PCT/EP2023/053903, filed Feb. 16, 2023, which designates the US and claims priority to Swedish Application No 2250181-1, filed Feb. 18, 2022 and to International Application No. PCT/EP2022/073784 filed Aug. 26, 2022, this application is also a continuation in part of International Application No. PCT/EP2022/073784, filed Aug. 26, 2022, which designates the US and claims priority to Swedish Application No 2250203-3, filed Feb. 18, 2022 and to International Application No PCT/EP2021/073893 filed Aug. 30, 2021, the entire contents of each of which are hereby incorporated by reference.

### TECHNICAL FIELD

The present invention relates to medical implants. More specifically the invention relates to medical implants for stretching the stomach wall of a patient for creating a feeling of satiety.

### BACKGROUND

When a person eats or drinks, the ingestion fills the stomach which will create a stretching of the stomach wall. The stretching activates stretch receptors which are predominantly situated in the fundus region of the stomach. The activation of stretch receptors creates a feeling of satiety in the patient, which reduces the patient's willingness to eat further.

### SUMMARY

According to one embodiment, a medical device for actively stretching a stomach wall of a patient for creating a sensation of satiety is provided. The medical device is implantable and comprises a first member configured to be fixated to a first portion of the stomach wall, and a second member configured to be fixated to a second portion of the stomach wall. The medical device further comprises an operation device for operating the second member to displace the second member in a first direction relative to the first member, for stretching the stomach wall between the first and second portion. At least one of the first and second member is flexible in a second direction substantially perpendicular to the first direction for adapting the medical device to the curvature of the stomach of the patient and/or facilitating insertion of the medical device into the body of the patient.

According to one embodiment, the operation device is further configured for operating the second member to displace the second member in a third direction relative to the first member, for stretching the stomach wall between the first and second portion.

According to one embodiment, the medical device further comprises a third member configured to be fixated to a third portion of the stomach wall. The operation device may further be configured for operating the third member to displace the third member relative to at least one of the first and second member, for stretching the stomach wall between the third portion and at least one of the first and second portions.

According to one embodiment, the medical device further comprises a fourth member configured to be fixated to a fourth portion of the stomach wall. The operation device may further be configured for operating the fourth member to displace the fourth member relative to at least one of the first, second and third member, for stretching the stomach wall between the fourth portion and at least one of the first, second and third portions.

According to one embodiment, at least one of the first, second, third and fourth member is inoperably fixated to a main portion. According to one embodiment at least two of the first, second, third and fourth members are inoperably fixated the main portion. According to one embodiment at least one of the first, second, third and fourth member is operably fixated to a main portion.

According to one embodiment, at least two of the first, second, third and fourth members are operably fixated the main portion.

The main portion may comprise at least part of the operation device.

At least one of the first, second, third and fourth member may comprise at least part of the operation device.

According to one embodiment, the member being inoperably fixated to the main portion may comprise at least part of the operation device.

According to one embodiment, the two members being inoperably fixated to the main portion each comprises at least part of the operation device.

According to one embodiment, the first member is inoperably fixated to the main portion and comprises a first portion of the operation device, and the second member is inoperably fixated to the main portion and comprises a second portion of the operation device. The first portion of the operation device may be configured for operating the third member and the second portion of the operation device may be configured for operating the fourth member.

According to one embodiment, the first member is inoperably fixated to the main portion and comprises a first portion of the operation device, and a second portion of the operation device, wherein the first portion of the operation device is configured for operating the third member and the second portion of the operation device is configured for operating the fourth member.

At least one of the first, second, third and fourth member may be elongated.

At least one of the first, second, third and fourth member may be hinged for enabling pivotal movement.

According to one embodiment, at least one of the first, second, third and fourth member may be configured to be at least partially invaginated by the tissue of the stomach wall.

According to one embodiment, at least one of the first, second, third and fourth member comprises at least one fixation portion configured for fixation of the member to tissue of the stomach wall using at least one of sutures, staplers or tissue growth promoting structure. The at least one fixation portion may comprise at least one of a through-hole and a recess for receiving the at least one of sutures or staplers.

The operation device in any if the embodiments may be a mechanical operation device and/or may be a hydraulic operation device.

According to one embodiment, the operation device comprises at least one electrical motor and the operation device may further comprise a gear system configured to reduce the speed and increase the force of the movement generated by the electrical motor.

According to one embodiment, the operation device is configured to be remotely powered.

The operation device may be remotely powered with mechanical force. The medical device may further comprise a receiving portion configured to receive mechanical force, and a transmission for transforming the received mechanical force into a force for displacing at least one of the members for stretching the stomach wall.

According to one embodiment, the receiving portion may be configured to receive a rotating mechanical force, and the transmission may be configured to transform the received rotating mechanical force into a liner mechanical force.

According to one embodiment, the receiving portion is configured to receive a linear mechanical force.

The operation device may in some embodiments be configured to be remotely powered with hydraulic force, and the medical device may is such embodiments further comprise a receiving portion configured to receive hydraulic force, and a transmission for transforming the received hydraulic force into a force for displacing at least one of the members for stretching the stomach wall. The transmission may comprise at least one hydraulic cylinder.

The medical device may further comprise an enclosure configured to enclose at least a portion of the operation device, and the enclosure may be flexible to enable the displacement of at least one member. The enclosure may comprise the main portion.

The medical device may further comprise an energy storage unit for directly or indirectly energizing the medical device and a controller for controlling the operation device.

The controller may comprise a wireless transceiver for communicating wirelessly with a source external to the body of the patient.

The medical device may further comprise at least one sensor, and the controller may be configured for receiving sensor input from the at least one sensor. The sensor may be a sensor configured to sense a physical parameter of the medical device system.

According to one embodiment, the sensor is a sensor configured to sense at least one of: a temperature of the medical device system, a parameter related to the power consumption of the medical device system, a parameter related to strain in the medical device system, a parameter related to a status of the energy storage unit, a parameter related to the wireless transfer of energy from a source external to the body of the patient, and a hydraulic pressure.

According to one embodiment, the sensor is a sensor configured to sense a physiological parameter of the patient and may be a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor is configured to sense a parameter related to the patient swallowing and may comprise at least one of: a motility sensor, a acoustic sensor, an optical sensor, or a strain sensor.

According to one embodiment, the sensor configured to sense pH is configured to sense the acidity in the stomach.

According to one embodiment, the controller is configured to transmit information based on sensor input to a source external to the body of the patient.

According to one embodiment, the controller is configured to transmit the information based on sensor input to a source external to the body of the patient, wirelessly.

The medical device could in any of the embodiments herein further comprise a capacitor connected to the implantable energy storage unit and connected to the operation device, the capacitor being configured to be charged by the implantable energy storage unit and to provide the operation device with electrical power.

The implantable energy storage unit may be a solid-state battery.

The implantable energy storage unit may be a tionyl-chlorid battery.

The medical device may further comprise a sensation generator adapted to generate a sensation detectable by a sense of the patient.

The controller may be configured to receive a patient generated control signal and the controller may be configured to control the operation device on the basis of the received patient generated control signal.

The controller may be configured to control the operation device on the basis of a signal related to a lapsed time or a time of day.

The controller may be configured to receive a signal from a sensor external to the body of the patient.

The medical device may in some embodiments further comprise an electrode arrangement configured to be arranged to engage and electrically stimulate muscle tissue of the stomach to exercise the muscle tissue to improve the conditions for long term implantation of the medical device.

A medical device system is further provided. The medical device system comprises a medical device according to any of the embodiments herein, and an implantable remote unit connected to the medical device.

The implantable remote unit may comprise an energy storage unit for directly or indirectly energizing the medical device. The implantable remote unit may comprise an energy receiver for wirelessly receiving energy from a source external to the body of the patient.

The medical device system may according to any one of the embodiments herein comprise a controller for controlling the medical device system. The controller may comprise a wireless transceiver for communicating wirelessly with a source external to the body of the patient. The implantable remote unit may further comprise at least one sensor, and wherein the controller may be configured for receiving sensor input from the at least one sensor.

The sensor may be a sensor configured to sense a physical parameter of the medical device system. The sensor may be a sensor configured to sense at least one of: a temperature of the medical device system, a parameter related to the power consumption of the medical device system, a parameter related to strain in the medical device system, a parameter related to a status of the energy storage unit, a parameter related to the wireless transfer of energy from a source external to the body of the patient, and a hydraulic pressure.

According to one embodiment, the sensor is a sensor configured to sense a physiological parameter of the patient and may be a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor is configured to sense a parameter related to the patient swallowing and may comprise at least one of: a motility sensor, a acoustic sensor, an optical sensor, or a strain sensor.

According to one embodiment, the sensor configured to sense pH is configured to sense the acidity in the stomach.

According to one embodiment, the controller is configured to transmit information based on sensor input to a source external to the body of the patient.

According to one embodiment, the controller is configured to transmit the information based on sensor input to a source external to the body of the patient, wirelessly.

The medical device could in any of the embodiments herein further comprise a capacitor connected to the implantable energy storage unit and connected to the operation device, the capacitor being configured to be charged by the implantable energy storage unit and to provide the operation device with electrical power.

The implantable energy storage unit may be a solid-state battery.

The implantable energy storage unit may be a tionyl-chlorid battery.

The medical device may further comprise a sensation generator adapted to generate a sensation detectable by a sense of the patient.

The controller may be configured to receive a patient generated control signal and the controller may be configured to control the operation device on the basis of the received patient generated control signal.

The controller may be configured to control the operation device on the basis of a signal related to a lapsed time or a time of day.

The controller may be configured to receive a signal from a sensor external to the body of the patient.

According to one embodiment, the implantable remote unit comprises at least a portion of the operation device. The portion of the operation device may comprise at least one electrical motor and may further comprise a gear system configured to reduce the speed and increase the force of the movement generated by the electrical motor. In some embodiments, the portion of the operation device comprises at least one hydraulic pump.

The medical device system may further comprise a force transferring element configured to mechanically transfer force from the implantable remote unit to the medical device. The force transferring element may be configured to transfer a rotating mechanical force from the implantable remote unit to the medical device and/or a linear mechanical force from the implantable remote unit to the medical device.

According to one embodiment, medical device system further comprises a force transferring element configured to hydraulically transfer force from the implantable remote unit to the medical device.

According to one embodiment, the medical device system further comprises at least one lead for transferring electrical energy and/or information from the implantable remote unit to the medical device.

According to one embodiment, the medical device system further comprises at least one injection port for injecting fluid into the medical device system.

A method of implanting a medical device for actively stretching a stomach wall of a patient for creating a sensation of satiety is further provided. The medical device comprises a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions. At least one of the first and second member is flexible in a second direction substantially perpendicular to the first direction. The method comprises making an incision in the abdomen of the patient, for accessing the stomach, dissecting a portion of the stomach, inserting the medical device into the abdomen of the patient, using the flexibility of at least one of the first and second member for adapting the medical device to the curvature of the stomach of the patient, and fixating the first member to a first portion of the stomach wall, and fixating the second member to a second portion of the stomach wall.

According to one embodiment, the medical device further comprises a third member configured to be fixated to a third portion of the stomach wall, and the method further comprises the step of fixating the third member to a third portion of the stomach wall.

According to one embodiment, the medical device further comprises a fourth member configured to be fixated to a fourth portion of the stomach wall, and the method comprises the step of fixating the fourth member to a fourth portion of the stomach wall.

According to one embodiment, at least one of the first, second, third and fourth members are connected to a main portion, and the method comprises using the flexibility of at least one of the first, second, third and fourth members for adapting the medical device to the curvature of the stomach of the patient by pivoting at least one of the first, second, third and fourth members relative to the main portion.

According to one embodiment, at least one of the steps of fixating the first member to a first portion of the stomach wall, and fixating the second member to a second portion of the stomach wall comprises at least partially invaginated at least one of the first and second member by the tissue of the stomach wall.

According to one embodiment, at least one of the steps of fixating the first member to a first portion of the stomach wall, and fixating the second member to a second portion of the stomach wall comprises fixating at least one of the first and second member to tissue of the stomach wall using at least one of sutures, staplers or tissue growth promoting structure.

The method may further comprise placing at least one sensor in the area of the esophagus of the patient for sensing a parameter related to the patient swallowing.

The step of placing at least one sensor in the area of the esophagus of the patient for sensing a parameter related to the patient swallowing may comprise placing at least one of: a motility sensor, a acoustic sensor, an optical sensor or a strain sensor.

According to one embodiment, placing at least one sensor in the area of the stomach of the patient comprises placing a sensor for sensing a parameter related to the patient eating.

According to one embodiment, the step of placing at least one sensor in the area of the stomach of the patient for sensing a parameter related to the patient eating comprises placing at least one of temperature sensor, blood saturation or oxygenation sensor, a blood pressure sensor, a sensor configured to sense a parameter related to an ischemia marker, or a pH sensor. The step of sensing pH may comprise sensing the acidity in the stomach.

According to one embodiment, the method may further comprise placing an electrode arrangement configured to be arranged between the medical device and the stomach to engage and electrically stimulate muscle tissue of the stomach to exercise the muscle tissue to improve the conditions for long term implantation of the medical device.

According to one embodiment, the method further comprises placing an implantable remote unit connected to the medical device in the body of the patient, and the step of placing an implantable remote unit connected to the medical device in the body of the patient may comprise placing an implantable remote unit comprising at least a portion of the operation device.

According to one embodiment, the step of placing an implantable remote unit connected to the medical device in the body of the patient may comprise placing an implantable remote unit comprising an energy storage unit.

According to one embodiment, the step of placing an implantable remote unit connected to the medical device in the body of the patient may comprise placing an implantable remote unit comprising a wireless energy receiver.

According to one embodiment, the step of placing an implantable remote unit connected to the medical device in the body of the patient may comprise placing an implantable remote unit comprising a wireless transceiver for wireless communication.

A method of implanting a medical device for actively stretching a stomach wall of a patient for creating a sensation of satiety is further provided. The medical device comprising a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions, wherein at least one of the first and second member is flexible in a second direction substantially perpendicular to the first direction. The method comprising making an incision in the abdomen of the patient, for accessing the stomach, dissecting a portion of the stomach, using the flexibility of at least one of the first and second member for facilitating insertion of the medical implant into the body of the patient, and inserting the medical device into the abdomen of the patient.

According to one embodiment, the step of using the flexibility of at least one of the first and second member for facilitating insertion of the medical device into the body of the patient further comprises using the flexibility of at least one of the first and second members for compressing the medical device such that it can be inserted through an incision in the skin of the patient.

According to one embodiment, the step of using the flexibility of at least one of the first and second member for facilitating insertion of the medical device into the body of the patient comprises using the flexibility of at least one of the first and second members for compressing the medical device such that it can be inserted through a cannula of a trocar and into the body of the patient.

According to one embodiment, the step of using the flexibility of at least one of the first and second member for expanding the medical device after the medical device has been inserted into the body of the patient.

According to one embodiment, the method further comprises the steps of fixating the first member to a first portion of the stomach wall, and fixating the second member to a second portion of the stomach wall.

According to one embodiment, the medical device further comprises a third member configured to be fixated to a third portion of the stomach wall, and the method further comprises the step of fixating the third member to a third portion of the stomach wall.

According to one embodiment, the medical device further comprises a fourth member configured to be fixated to a fourth portion of the stomach wall, and the method comprises the step of fixating the fourth member to a fourth portion of the stomach wall.

According to one embodiment, at least one of the first, second, third and fourth members are connected to a main portion, and the method comprises using flexibility of at least one of the first, second, third and fourth members by pivoting at least one of the first, second, third and fourth members relative to the main portion.

According to one embodiment, at least one of the steps of fixating the first member to a first portion of the stomach wall, and fixating the second member to a second portion of the stomach wall comprises at least partially invaginating at least one of the first and second member by the tissue of the stomach wall. At least one of the steps of fixating the first member to a first portion of the stomach wall, and fixating the second member to a second portion of the stomach wall may comprise fixating at least one of the first and second member to tissue of the stomach wall using at least one of sutures, staplers or tissue growth promoting structure.

The method may further comprise placing at least one sensor in the area of the esophagus of the patient for sensing a parameter related to the patient swallowing. The step of placing at least one sensor in the area of the esophagus of the patient for sensing a parameter related to the patient swallowing may comprise placing at least one of: a motility sensor, a acoustic sensor, an optical sensor or a strain sensor.

According to one embodiment, the method further comprises placing at least one sensor in the area of the stomach of the patient for sensing a parameter related to the patient eating.

The step of placing at least one sensor in the area of the stomach of the patient for sensing a parameter related to the patient eating may comprise placing at least one of temperature sensor, blood saturation or oxygenation sensor, a blood pressure sensor, a sensor configured to sense a parameter related to an ischemia marker, or a pH sensor. The step of sensing pH comprises sensing the acidity in the stomach.

According to one embodiment, the method further comprises placing an electrode arrangement configured to be arranged between the medical device and the stomach to engage and electrically stimulate muscle tissue of the stomach to exercise the muscle tissue to improve the conditions for long term implantation of the medical device.

According to one embodiment, the method further comprises placing an implantable remote unit connected to the medical device in the body of the patient.

The step of placing an implantable remote unit connected to the medical device in the body of the patient may comprise placing an implantable remote unit comprising at least a portion of the operation device.

The step of placing an implantable remote unit connected to the medical device in the body of the patient may comprise placing an implantable remote unit comprising an energy storage unit.

The step of placing an implantable remote unit connected to the medical device in the body of the patient may comprise placing an implantable remote unit comprising a wireless energy receiver.

The step of placing an implantable remote unit connected to the medical device in the body of the patient may comprise placing an implantable remote unit comprising a wireless communication unit.

A method of calibrating a medical device for actively stretching a stomach wall of a patient for creating a sensation of satiety is further provided. The medical device comprising a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions, and a controller comprising a transceiver for wireless communication. The method comprising: receiving, at the controller, a first input signal comprising at least one of a sensor input signal related to a physical parameter of the medical device, and an input signal from a source external to the body of the patient. The method further comprises controlling, by the controller, the operation device to adjust the stretching of the stomach wall, in response to the first input signal, and receiving, at the transceiver, a second input signal from the source external to the patient, and controlling, by the controller, the operation device to further adjust the stretching of the stomach wall, in response to the second input signal.

According to one embodiment, the sensor input signal related to a physical parameter of the medical device is a sensor input related to at least one of: energy consumption, position of at least one of the first member, the second member and the operation device, strain on at least one of the first member, the second member and the operation device, and speed of the operation device.

According to one embodiment, the input signal from a source external to the body of the patient is an input signal generated by the patient.

According to one embodiment, the input signal generated by the patient is an input signal related to at least one of: a feeling of satiety experienced by the patient, a feeling of pain experienced by the patient, and the patient ingesting something.

According to one embodiment, the step of adjusting the stretching of the stomach wall comprises adjusting one of: the force with which the stomach wall is stretched, the speed with which the stomach wall is stretched, and the duration of the stretching of the stomach wall.

A medical device for actively stretching a stomach wall of a patient for creating a sensation of satiety is further provided. The medical device being implantable and comprising: a first member comprising a first stomach engager configured to be fixated to a first portion of the stomach wall, a second member comprising a second stomach engager portion configured to be fixated to a second portion of the stomach wall, an operation device for operating the second member to displace the second member in a first direction relative to the first member, for stretching the stomach wall between the first and second portions, and a main portion. The first and second members are connected to the main portion, and at least the second member is pivotally connected to the main portion, such that the second member can be displaced in the first direction relative to the first member by the second member pivoting in relation to the main portion. The operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.

According to one embodiment, the at least one eccentric rotatable engagement member is configured to engage the second member at a distance from the point of pivot of the second member.

According to one embodiment, the eccentric rotatable engagement member comprises at least one of a recess or protrusion, and the second member comprises at least one of a recess or protrusion. The engagement between the recess or protrusion of the engagement member and the recess or protrusion of the second member causes the second member to pivot in relation to the main portion for displacing the second member in the first direction relative to the first member.

According to one embodiment, the first member is pivotally connected to the main portion, such that the first member can be displaced relative to at least one of the second member and a third member, by the first member pivoting in relation to the main portion.

According to one embodiment, the operation device is configured to simultaneously displace the second member in the first direction and the first member in a second direction to thereby increase the distance between the first stomach engager portion and the second stomach engager portion to stretch the stomach wall between the first portion of the stomach wall and the second portion of the stomach wall.

According to one embodiment, the operation device comprises a radial engagement surface configured to radially engage at least the second member such that the eccentric rotation of the engagement member causes the second member to pivot in relation to the main portion.

According to one embodiment, at least the second member is configured to be operated in the first direction by the eccentric rotation of the engagement member and in an opposite direction by an elastic element.

According to one embodiment, the eccentric rotatable engagement member comprises a groove encircling the center of rotation of the eccentric rotatable engagement member. The groove may be configured to control the movement of at least the second member in the first direction and in the opposite direction. The groove may be configured to control the movement of at least the second member at all times.

According to one embodiment, the medical device comprises the first member, the second member, the third member and a fourth member, and wherein: the first, second, third and fourth members are all pivotally connected to the main portion, the groove is configured to control the movement of the first, second, third and fourth members in a first direction and in an opposite direction, and as the eccentric rotatable engagement member comprising the groove rotates, the first, second, third and fourth members are sequentially displaced in relation to the main portion in the first direction and in the opposite direction for sequentially stretching the stomach wall portions between the members.

According to one embodiment, as the eccentric rotatable engagement member comprising the groove rotates: the first and second members are simultaneously displaced, or the second and third members are simultaneously displaced, or the third and fourth members are simultaneously displaced.

According to one embodiment, at least one of the first, second, third and fourth member is configured to be at least partially invaginated by the tissue of the stomach wall.

According to one embodiment, at least one of the first, second, third and fourth member comprises at least one fixation portion configured for fixation of the member to tissue of the stomach wall using at least one of sutures, staplers or tissue growth promoting structure. The at least one fixation portion may comprise at least one of a through-hole and a recess for receiving the at least one of sutures or staplers.

The operation device in any of the embodiments herein may comprise at least one electrical motor which may be connected to a gear system configured to reduce the speed and increase the force of the movement generated by the electrical motor.

According to one embodiment, the operation device is configured to be remotely powered.

The operation device may be configured to be remotely powered with mechanical force.

The medical device may further comprise an enclosure configured to enclose at least a portion of the operation device, and the enclosure may be flexible to enable the displacement of at least one member. The enclosure may comprise the main portion.

The medical device may further comprise an energy storage unit for directly or indirectly energizing the medical device and a controller for controlling the operation device.

The controller may comprise a wireless transceiver for communicating wirelessly with a source external to the body of the patient.

The medical device may further comprise at least one sensor, and the controller may be configured for receiving sensor input from the at least one sensor. The sensor may be a sensor configured to sense a physical parameter of the medical device system.

According to one embodiment, the sensor is a sensor configured to sense at least one of: a temperature of the medical device system, a parameter related to the power consumption of the medical device system, a parameter related to strain in the medical device system, a parameter related to a status of the energy storage unit, a parameter related to the wireless transfer of energy from a source external to the body of the patient, and a hydraulic pressure.

According to one embodiment, the sensor is a sensor configured to sense a physiological parameter of the patient and may be a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor is configured to sense a parameter related to the patient swallowing and may comprise at least one of: a motility sensor, a acoustic sensor, an optical sensor, or a strain sensor.

According to one embodiment, the sensor configured to sense pH is configured to sense the acidity in the stomach.

According to one embodiment, the controller is configured to transmit information based on sensor input to a source external to the body of the patient.

According to one embodiment, the controller is configured to transmit the information based on sensor input to a source external to the body of the patient, wirelessly.

The medical device could in any of the embodiments herein further comprise a capacitor connected to the implantable energy storage unit and connected to the operation device, the capacitor being configured to be charged by the implantable energy storage unit and to provide the operation device with electrical power.

The implantable energy storage unit may be a solid-state battery.

The implantable energy storage unit may be a tionyl-chlorid battery.

The medical device may further comprise a sensation generator adapted to generate a sensation detectable by a sense of the patient.

The controller may be configured to receive a patient generated control signal and the controller may be configured to control the operation device on the basis of the received patient generated control signal.

The controller may be configured to control the operation device on the basis of a signal related to a lapsed time or a time of day.

The controller may be configured to receive a signal from a sensor external to the body of the patient.

The medical device may in some embodiments further comprise an electrode arrangement configured to be arranged to engage and electrically stimulate muscle tissue of the stomach to exercise the muscle tissue to improve the conditions for long term implantation of the medical device.

A medical device for actively stretching a stomach wall of a patient for creating a sensation of satiety is further provided. The medical device being implantable and comprising first member comprising a first stomach engager configured to be fixated to a first portion of the stomach wall and a second member comprising a second stomach engager portion configured to be fixated to a second portion of the stomach wall. The medical device further comprises a hydraulic operation device for operating the second member to displace the second member in a first direction relative to the first member, for stretching the stomach wall between the first and second portions, and a main portion. The first and second members are connected to the main portion, and at least the second member is configured to bend or pivot in relation to the main portion, such that the second member can be displaced in the first direction relative to the first member by the second member bending or pivoting in relation to the main portion. At least the second member comprises a fluid chamber at least partially enclosed by a flexible wall portion, and the flexible wall portion causes the second member to bend or pivot when hydraulic fluid is moved to or from the fluid chamber for stretching the stomach wall between the first and second portions.

According to one embodiment, the flexible wall portion comprises elevated and lowered portions. The elevated and lowered portions may be configured to enable the second member to bend or pivot when fibrotic tissue is covering the elevated and lowered portions.

The flexible wall portion comprises at least one pleated portion such as a bellows.

According to one embodiment, the second member comprises a pivoting element pivotally fixated to the main portion, the flexible wall portion causes the pivoting element to pivot when hydraulic fluid is moved to or from the fluid chamber for stretching the stomach wall between the first and second portions.

According to one embodiment, the second member comprises a second fluid chamber partially enclosed by a flexible wall portion, and wherein the bending or pivoting of the second member is caused by the altering the relationship between the fluid levels in the first and second fluid chamber.

According to one embodiment, the bending or pivoting of the second member is caused by at least one of: movement of fluid from the first fluid chamber, and movement of fluid to the second fluid chamber.

According to one embodiment, the second member is configured to be displaced: in the first direction relative to the first member by the movement of fluid from the first fluid chamber, and in a second direction relative to the first member by the movement of fluid from the second fluid chamber.

According to one embodiment, the second member is configured to be displaced: in the first direction relative to the first member by the movement of fluid to the first fluid chamber, and in a second direction relative to the first member by the movement of fluid to the second fluid chamber.

The first member may comprise a fluid chamber at least partially enclosed by a flexible wall portion, and wherein the flexible wall portion may cause the first member to bend or pivot when hydraulic fluid is moved to or from the fluid chamber.

According to one embodiment, the medical device comprises at least one of a third member comprising a fluid chamber at least partially enclosed by a flexible wall portion, and wherein the flexible wall portion causes the third member to bend or pivot when hydraulic fluid is moved to or from the fluid chamber, a fourth member comprising a fluid chamber at least partially enclosed by a flexible wall portion, and wherein the flexible wall portion causes the fourth member to bend or pivot when hydraulic fluid is moved to or from the fluid chamber, and a fifth member comprising a fluid chamber at least partially enclosed by a flexible wall portion, and wherein the flexible wall portion causes the fifth member to bend or pivot when hydraulic fluid is moved to or from the fluid chamber.

According to one embodiment, the medical device further comprises at least one operable valve configured to operate to at least one of: switch between distributing hydraulic fluid to or from the fluid chamber of the first member and to or from the fluid chamber of the second member, switch between distributing hydraulic fluid to or from the fluid chamber of the first member and to or from the fluid chamber of the second member and to or from the fluid chamber of the third member, switch between distributing hydraulic fluid to or from the fluid chamber of the first member and to or from the fluid chamber of the second member and to or from the fluid chamber of the third member and to or from the fluid chamber of the fourth member, and switch between distributing hydraulic fluid to or from the fluid chamber of the first member and to or from the fluid chamber of the second member and to or from the fluid chamber of the third member and to or from the fluid chamber of the fourth member and to or from the fluid chamber of the fifth member.

According to one embodiment, the at least one operable valve is a rotatable operable valve.

According to one embodiment, the medical device further comprises an actuator for operating the operable valve, and the actuator is at least one of: an electric actuator and a hydraulic actuator.

According to one embodiment, the operable valve is configured to simultaneously distribute fluid to the first fluid chamber of the first member and the second fluid chamber of the second member, for causing the first and second members to bend or pivot in different directions, away from each other, for stretching the stomach wall between the first and second member.

According to one embodiment, the operable valve is configured to simultaneously distribute fluid to the first fluid chamber of the second member and the second fluid chamber of the third member, for causing the second and third members to bend or pivot in different directions, away from each other, for stretching the stomach wall between the second and third member.

According to one embodiment, each of the first, second, third, fourth and fifth member comprises a stomach engager portion. The stomach engager portion may be configured to be invaginated by the stomach wall using stomach-to-stomach sutures or staplers.

The stomach engager portion may comprise at least one of a fixation portion configured for fixation of the stomach engager portion to tissue of the stomach wall using at least one of sutures and staplers, and a tissue growth promoting structure such that the stomach engager portion can be fixated to the stomach wall by in-growth of fibrotic tissue.

According to one embodiment, the main portion comprises a fluid conduit for conducting fluid to at least one of the first, second, third, fourth and fifth member.

According to one embodiment, the medical device further comprises an implantable hydraulic pump for pumping fluid to the fluid conduit. The implantable pump may be configured to be positioned at a remote location in relation to the main portion.

The medical device may further comprises an energy storage unit for directly or indirectly energizing the medical device and a controller for controlling at least one of the pump and the operable valve.

The controller may comprise a wireless transceiver for communicating wirelessly with a source external to the body of the patient.

The medical device may further comprise at least one sensor, and the controller may be configured for receiving sensor input from the at least one sensor. The sensor may be a sensor configured to sense a physical parameter of the medical device system.

According to one embodiment, the sensor is a sensor configured to sense at least one of: a temperature of the medical device system, a parameter related to the power consumption of the medical device system, a parameter related to strain in the medical device system, a parameter related to a status of the energy storage unit, a parameter related to the wireless transfer of energy from a source external to the body of the patient, and a hydraulic pressure.

According to one embodiment, the sensor is a sensor configured to sense a physiological parameter of the patient and may be a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor is configured to sense a parameter related to the patient swallowing and may comprise at least one of: a motility sensor, a acoustic sensor, an optical sensor, or a strain sensor.

According to one embodiment, the sensor configured to sense pH is configured to sense the acidity in the stomach.

According to one embodiment, the controller is configured to transmit information based on sensor input to a source external to the body of the patient.

According to one embodiment, the controller is configured to transmit the information based on sensor input to a source external to the body of the patient, wirelessly.

The medical device could in any of the embodiments herein further comprise a capacitor connected to the implantable energy storage unit and connected to the operation device, the capacitor being configured to be charged by the implantable energy storage unit and to provide the operation device with electrical power.

The implantable energy storage unit may be a solid-state battery.

The implantable energy storage unit may be a tionyl-chlorid battery.

The medical device may further comprise a sensation generator adapted to generate a sensation detectable by a sense of the patient.

The controller may be configured to receive a patient generated control signal and the controller may be configured to control the operation device on the basis of the received patient generated control signal.

The controller may be configured to control the operation device on the basis of a signal related to a lapsed time or a time of day.

The controller may be configured to receive a signal from a sensor external to the body of the patient.

The medical device may in some embodiments further comprise an electrode arrangement configured to be arranged to engage and electrically stimulate muscle tissue of the stomach to exercise the muscle tissue to improve the conditions for long term implantation of the medical device.

A medical device for actively stretching a stomach wall of a patient for creating a sensation of satiety is further provided. The medical device being implantable and comprising: a first member comprising a first stomach engager configured to be fixated to a first portion of the stomach wall and a second member comprising a second stomach engager portion configured to be fixated to a second portion of the stomach wall. The medical device further comprises an operation device for operating the second member to displace the second member in a first direction, for stretching the stomach wall between the first and second portions, and a main portion. The first and second members are connected to the main portion, and at least the second member is configured to bend or pivot in relation to the main portion, such that the second member can be displaced in a direction away from the first member by the second member bending or pivoting in relation to the main portion. The operation device comprises a first flexible cable for pulling on the second member for displacing the second member in the direction away from the first member, and the first flexible cable is fixated to the second member at the distal half of thereof.

According to one embodiment, at least the second member comprises a flexible wall portion, and wherein the first flexible cable is at least partially enclosed by the flexible wall portion. The flexible wall portion may comprise elevated and lowered portions and the elevated and lowered portions may be configured to enable the second member to bend or pivot when fibrotic tissue is covering the elevated and lowered portions.

According to one embodiment the flexible wall portion comprises at least one pleated portion, such as a bellows.

According to one embodiment, the second member comprises a pivoting element pivotally fixated to the main portion, and wherein the first flexible cable causes the pivoting element to pivot when operated for stretching the stomach wall between the first and second portions.

According to one embodiment, the operation device comprises a second flexible cable for pulling on the second member for displacing the second member in a direction towards the first member, and the second flexible cable is fixated to the second member at the distal half thereof.

According to one embodiment, the first member is configured to bend or pivot in relation to the main portion, such that the first member can be displaced in a direction away from the second member by the first member bending or pivoting in relation to the main portion.

According to one embodiment, the first flexible cable is further fixated to the first member, such that pulling on the first flexible cable causes the second member to displace in the direction away from the first member, and the first member to displace in the direction away from the second member, for stretching the stomach wall between the first and second portions.

According to one embodiment, the medical device comprises at least one of a third member configured to bend or pivot in relation to the main portion, a fourth member configured to bend or pivot in relation to the main portion, and a fifth member configured to bend or pivot in relation to the main portion.

According to one embodiment, a second cable is connected to the second and third members, at the distal half thereof, such that pulling on the cable causes the second member to displace in a direction away from the third member, and the third member to displace in a direction away from the second member.

According to one embodiment, a third cable is connected to the third and fourth members, at the distal half thereof, such that pulling on the cable causes the third member to displace in a direction away from the fourth member, and the fourth member to displace in a direction away from the third member.

According to one embodiment, a fourth cable is connected to the fourth and fifth members, at the distal half thereof, such that pulling on the cable causes the fourth member to displace in a direction away from the fifth member, and the fifth member to displace in a direction away from the fourth member.

According to one embodiment, a fifth cable is connected to the fifth and first members, at the distal half thereof, such that pulling on the cable causes the fifth member to displace in a direction away from the first member, and the first member to displace in a direction away from the fifth member.

According to one embodiment, the medical device further comprises an operation device configured to pull on at least one of: the first cable, the second cable, the third cable, the fourth cable, and the fifth cable.

According to one embodiment, the operation device is configured to at least one of: switch between pulling on the first cable and the second cable, switch between pulling on the second cable and the third cable, switch between pulling on the third cable and the fourth cable, and switch between pulling on the fourth cable and the fifth cable.

The operation device may be a rotatable operation device.

The operation device may comprise at least one eccentric rotatable engagement member configured to engage at least the first flexible cable for pulling the first flexible wire.

The eccentric rotatable engagement member may further be configured to engage at least one of: the second flexible cable, the third flexible cable, the fourth flexible cable, and the fifth flexible cable.

According to one embodiment, the eccentric rotatable engagement member comprises at least one of a recess or protrusion, and at least the first flexible cable is fixated to at least one of a recess or protrusion, and the engagement between the recess or protrusion of the engagement member and the recess or protrusion of the second member causes the second member to pivot in relation to the main portion for displacing the second member in the first direction relative to the first member.

According to one embodiment, at least the second member is configured to be operated in the first direction by the first flexible cable and in an opposite direction by an elastic element.

According to one embodiment, the eccentric rotatable engagement member comprises a groove encircling the center of rotation of the eccentric rotatable engagement member.

According to one embodiment, the groove is configured to control the movement of at least the second member in the first direction and in the opposite direction.

The stomach engager portions may in any of the embodiments herein be configured to be invaginated by the stomach wall using stomach-to-stomach sutures or staplers.

The stomach engager portions may comprise at least one of: a fixation portion configured for fixation of the stomach engager portion to tissue of the stomach wall using at least one of sutures and staplers, and a tissue growth promoting structure such that the stomach engager portion can be fixated to the stomach wall by in-growth of fibrotic tissue.

The operation device may in any of the embodiments herein comprises at least one electrical motor which may be connected to a gear system configured to reduce the speed and increase the force of the movement generated by the electrical motor.

The operation device may partially be positioned at a remote location in the body of the patient, in relation to the main portion.

The medical device may further comprises an energy storage unit for directly or indirectly energizing the medical device and a controller for controlling at least one of the pump and the operable valve.

The controller may comprise a wireless transceiver for communicating wirelessly with a source external to the body of the patient.

The medical device may further comprise at least one sensor, and the controller may be configured for receiving sensor input from the at least one sensor. The sensor may be a sensor configured to sense a physical parameter of the medical device system.

According to one embodiment, the sensor is a sensor configured to sense at least one of: a temperature of the medical device system, a parameter related to the power consumption of the medical device system, a parameter related to strain in the medical device system, a parameter related to a status of the energy storage unit, a parameter related to the wireless transfer of energy from a source external to the body of the patient, and a hydraulic pressure.

According to one embodiment, the sensor is a sensor configured to sense a physiological parameter of the patient and may be a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor is configured to sense a parameter related to the patient swallowing and may comprise at least one of: a motility sensor, a acoustic sensor, an optical sensor, or a strain sensor.

According to one embodiment, the sensor configured to sense pH is configured to sense the acidity in the stomach.

According to one embodiment, the controller is configured to transmit information based on sensor input to a source external to the body of the patient.

According to one embodiment, the controller is configured to transmit the information based on sensor input to a source external to the body of the patient, wirelessly.

The medical device could in any of the embodiments herein further comprise a capacitor connected to the implantable energy storage unit and connected to the operation device, the capacitor being configured to be charged by the implantable energy storage unit and to provide the operation device with electrical power.

The implantable energy storage unit may be a solid-state battery.

The implantable energy storage unit may be a tionyl-chlorid battery.

The medical device may further comprise a sensation generator adapted to generate a sensation detectable by a sense of the patient.

The controller may be configured to receive a patient generated control signal and the controller may be configured to control the operation device on the basis of the received patient generated control signal.

The controller may be configured to control the operation device on the basis of a signal related to a lapsed time or a time of day.

The controller may be configured to receive a signal from a sensor external to the body of the patient.

The medical device may in some embodiments further comprise an electrode arrangement configured to be arranged to engage and electrically stimulate muscle tissue of the stomach to exercise the muscle tissue to improve the conditions for long term implantation of the medical device.

A medical device for actively stretching a stomach wall of a patient for creating a sensation of satiety is further provided. The medical device being implantable and comprising a first member comprising a first stomach engager configured to engage a first portion of the stomach wall, a second member comprising a second stomach engager portion configured to engage a second portion of the stomach wall, and a main portion. The first and second members are connected to the main portion.

The medical device further comprises an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.

According to one embodiment, the second member is configured to bend or pivot in relation to the main portion, such that the second member can be displaced in a direction away from the first member.

According to one embodiment, the first member is configured to bend or pivot in relation to the main portion, such that the first member can be displaced in a direction away from the second member.

According to one embodiment, the medical device further comprises a third member configured to engage a third portion of the stomach wall.

According to one embodiment, the medical device further comprises a fourth member configured to engage a fourth portion of the stomach wall.

According to one embodiment, the operation device is further configured for operating at least one of the first, third and fourth member to displace one of the first, third and fourth member relative to the main portion for stretching the stomach wall.

According to one embodiment, the main portion comprises at least part of the operation device.

According to one embodiment, at least one of the first, second, third and fourth member comprises at least part of the operation device.

According to one embodiment at least one of the first, second, third and fourth member is elongated.

According to one embodiment, at least one of the first, second, third and fourth member is hinged for enabling pivotal movement.

According to one embodiment, at least one of the first, second, third and fourth member is configured to be at least partially invaginated by the tissue of the stomach wall.

According to one embodiment, the main portion is configured to be at least partially invaginated by the tissue of the stomach wall.

According to one embodiment, at least one of the first, second, third and fourth member comprises at least one fixation portion configured for fixation of the member to tissue of the stomach wall using at least one of sutures, staplers or tissue growth promoting structure.

According to one embodiment, the portion of the operation device placed in the remote unit comprises at least one electrical motor.

According to one embodiment, the portion of the operation device placed in the remote unit further comprises a gear system configured to reduce the speed and increase the force of the movement generated by the electrical motor.

According to one embodiment, a portion of the operation device is placed in the main portion, and the portion of the operation device placed in the main portion comprises a receiving portion configured to receive mechanical force, and a transmission for transforming the received mechanical force into a force for displacing at least one of the members for stretching the stomach wall. The transmission may comprise a gear system configured to reduce the speed and increase the force of the received mechanical force.

According to one embodiment, the receiving portion is configured to receive a rotating mechanical force, and the transmission is configured to transform the received rotating mechanical force into a liner mechanical force.

According to one embodiment, the receiving portion is configured to receive a linear mechanical force.

According to one embodiment, a portion of the operation device is placed in the main portion. The portion of the operation device placed in the main portion comprises a receiving portion configured to receive hydraulic force, and a transmission for transforming the received hydraulic force into a force for displacing at least one of the members for stretching the stomach wall.

According to one embodiment, the transmission comprises at least one hydraulic cylinder.

According to one embodiment, the portion of the operation device placed in the main portion comprises at least one operable valve.

The portion of the operation device placed in the remote unit may comprise at least one operable valve.

According to one embodiment, the operable valve is configured to control the flow of hydraulic fluid from a first state in which the hydraulic fluid can operate the first member to a second state in which the hydraulic fluid can operate the second member. The portion of the operation device placed in the remote unit may comprise at least one hydraulic pump. The remote unit may comprise a hydraulic reservoir for holding a hydraulic fluid. The remote unit may comprise an injection port for at least one of injecting and removing hydraulic fluid from the medical device.

According to one embodiment, the remote unit further comprises an energy storage unit for directly or indirectly energizing the medical device.

According to one embodiment, the remote unit further comprises a controller for controlling the operation device. The controller may comprise a wireless transceiver for communicating wirelessly with a source external to the body of the patient.

According to one embodiment, the medical device comprises at least one sensor, and the controller may be configured for receiving sensor input from the at least one sensor.

According to one embodiment, the sensor is a sensor configured to sense a physical parameter of the medical device system.

According to one embodiment, the sensor is a sensor configured to sense at least one of: a temperature of the medical device system, a parameter related to the power consumption of the medical device system, a parameter related to strain in the medical device system, a parameter related to a status of the energy storage unit, a parameter related to the wireless transfer of energy from a source external to the body of the patient, a hydraulic pressure.

According to one embodiment, the sensor is a sensor configured to sense a physiological parameter of the patient and may be a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor is configured to sense a parameter related to the patient swallowing and may comprise at least one of: a motility sensor, a acoustic sensor, an optical sensor, or a strain sensor.

According to one embodiment, the sensor configured to sense pH is configured to sense the acidity in the stomach.

According to one embodiment, the controller is configured to transmit information based on sensor input to a source external to the body of the patient.

According to one embodiment, the controller is configured to transmit the information based on sensor input to a source external to the body of the patient, wirelessly.

The remote unit could in any of the embodiments herein further comprise a capacitor connected to the implantable energy storage unit and connected to the operation device, the capacitor being configured to be charged by the implantable energy storage unit and to provide the operation device with electrical power.

The remote unit may further comprise a sensation generator adapted to generate a sensation detectable by a sense of the patient.

The controller may be configured to receive a patient generated control signal and the controller may be configured to control the operation device on the basis of the received patient generated control signal.

The controller may be configured to control the operation device on the basis of a signal related to a lapsed time or a time of day.

The medical device could further comprises at least one lead for transferring electrical energy and/or information from the remote unit to the main portion.

According to one embodiment, the medical device further comprises a housing configured to enclose the remote unit. A first portion of the housing may be made from titanium and a second portion of the housing may be made from a ceramic material.

According to one embodiment, the portion of the housing made from a ceramic material comprises at least one coil embedded in the ceramic material.

According to one embodiment, the portion of the housing made from a ceramic material comprises at least one lead embedded in the ceramic material, for transferring electrical energy and/or information through the enclosure.

According to one embodiment, the remote unit comprises a magnetic coupling for transferring mechanical through the housing.

The remote unit may in any of the embodiments herein comprise a first portion configured to be placed on a first side of the tissue portion, the first portion having a first cross-sectional area in a first plane and comprises a first surface configured to engage a first tissue surface of the tissue portion, a second portion configured to be placed on a second side of the tissue portion, the second portion having a second cross-sectional area in a second plane and comprises a second surface configured to engage a second tissue surface of the tissue portion, and a connecting portion configured to be placed through a hole in the tissue portion, the connecting portion having a third cross-sectional area in a third plane and is configured to connect the first portion to the second portion. The first, second and third planes are parallel to each other and the third cross-sectional area is smaller than the first and second cross-sectional areas, such that the first and second portions are prevented from travelling through the hole in the tissue portion in a direction perpendicular to the first, second and third planes.

According to one embodiment, the first portion comprises a first wireless energy receiver for receiving energy transmitted wirelessly from an external wireless energy transmitter, and an internal wireless energy transmitter configured to transmit energy wirelessly to the second portion, and the second portion comprises a second wireless energy receiver for receiving energy from the first portion wirelessly.

According to one embodiment, the remote unit comprises a first energy storage unit connected to the first wireless energy receiver, and the second portion comprises a second energy storage unit connected to the second wireless energy receiver.

According to one embodiment, the first wireless energy receiver is configured to receive energy from the external wireless energy transmitter and store the received energy in the first energy storage unit, the wireless energy transmitter is configured to transmit energy stored in the first energy storage unit to the second wireless energy receiver, and the second wireless energy receiver is configured to receive energy from the internal wireless energy transmitter and store the received energy in the second energy storage unit.

According to one embodiment, the first portion comprises a first wireless communication receiver for receiving wireless communication from an external device, and the first portion comprises a first wireless communication transmitter for transmitting wireless communication to a second wireless communication receiver in the second portion.

According to one embodiment, the first portion is detachably connected to at least one of the second portion and the connecting portion.

According to one embodiment, the connecting portion comprising a flange having a flange area being larger than the third cross-sectional area, such that the flange is hindered from travelling through the hole in the tissue portion, such that the second portion and the connecting portion can be held in position by the tissue portion of the patient also when the first portion is disconnected from the connecting portion.

The connecting portion may in any of the embodiments herein comprise a conduit for transferring a fluid from the first portion to the second portion.

A medical device for actively stretching a stomach wall of a patient for creating a sensation of satiety is further provided. The medical device being implantable and comprising a first member comprising a closed curve, the first member comprising a first portion comprising a first stomach engager configured to engage a first portion of the stomach wall and a second portion comprising a second stomach engager configured to engage a second portion of the stomach wall, wherein the first member is operable such that the first and second portion can move towards each other or away from each other for stretching a portion of the stomach wall.

According to one embodiment, the first member is configured to encircle a portion of the stomach wall of the patient.

According to one embodiment, the first member comprises a simple closed curve.

According to one embodiment, the first member is ring shaped.

According to one embodiment, the first member is operable to assume an oval shape.

According to one embodiment, the first member is operable to assume an elliptical shape.

According to one embodiment, the first and second portions moving away from each other causes a stretching of a portion of the stomach wall between the first and second portions.

According to one embodiment, the first member further comprises a third portion comprising a third stomach engager configured to engage a third portion of the stomach wall and a fourth portion comprising a fourth stomach engager configured to engage a fourth portion of the stomach wall, and wherein the first and second portions moving towards each other causes the third and fourth portions to move away from each other.

According to one embodiment, the third and fourth portions moving away from each other causes a stretching of a portion of the stomach wall between the third and fourth portions.

According to one embodiment, the first member is configured to be operated to stretch the stomach wall between the first member and a second member fixated to the stomach wall.

According to one embodiment, the first and second portions moving towards each causes a stretching of the stomach wall between at least one of the first and second portion, and the second member.

According to one embodiment, the medical device further comprises a second member configured to act as a support against the force created by the operation of the first member, such that the stomach wall between the first and second member can be stretched.

According to one embodiment, the support is configured to be fixated to the stomach wall of the patient. The support may comprise a closed curve.

According to one embodiment, the first member is mechanically operable and may in such cases comprise a mechanical operation device. The mechanical operation device may comprise at least one flexible element, directly or indirectly fixated to the first and second portion of the first member. The flexible element is configured to operate the first member for at least one of: moving the first portion towards the second portion, and moving the first portion away from the second portion.

According to one embodiment, the mechanical operation device may comprise at least one second flexible element, directly or indirectly fixated to the third and fourth portion of the first member. The flexible element may be configured to operate the first member for at least one of: moving the third portion towards the fourth portion, and moving the third portion away from the fourth portion.

According to one embodiment, the mechanical operation device comprises at least one third flexible element, directly or indirectly fixated to a fifth and sixth portion of the first member. The flexible element may be configured to operate the first member for at least one of: moving the fifth portion towards the sixth portion, and moving the fifth portion away from the sixth portion.

According to one embodiment, the mechanical operation device comprises at least one fourth flexible element, directly or indirectly fixated to a seventh and eight portion of the first member. The flexible element may be configured to operate the first member for at least one of: moving the seventh portion towards the eight portion, and moving the seventh portion away from the eight portion.

According to one embodiment, at least one of the first and second members comprises at least one recess or channel, and wherein at least one of the flexible elements travels at least partially in the recess or channel.

According to one embodiment, a first portion of the flexible element is directly or indirectly fixated to the first portion of the first member, and a second portion of the flexible element is directly or indirectly fixated to the second portion of the first member, such that operation of the flexible element simultaneously operates the first and second portion of the first member.

The medical device may further comprise at least one sheath, and the least one flexible element may be configured to travel through the sheath.

According to one embodiment, the sheath comprises elevated and lowered portions, such that the sheath can remain flexible even as fibrotic tissue overgrows the sheath.

According to one embodiment, the first member is hydraulically operable and in such cases the medical device may further comprise a hydraulic operation device.

According to one embodiment, the hydraulic operation device may comprise at least one hydraulic actuator, directly or indirectly fixated to the first and second portion of the first member. The hydraulic actuator may be configured to operate the first member for at least one of: moving the first portion towards the second portion, and moving the first portion away from the second portion.

According to one embodiment, the hydraulic operation device comprises at least one second hydraulic actuator, directly or indirectly fixated to the third and fourth portion of the first member, and wherein the hydraulic actuator is configured to operate the first member for at least one of: moving the third portion towards the fourth portion, and moving the third portion away from the fourth portion.

According to one embodiment, the hydraulic operation device comprises at least one third hydraulic actuator, directly or indirectly fixated to a fifth and sixth portion of the first member, and wherein the hydraulic actuator is configured to operate the first member for at least one of: moving the fifth portion towards the sixth portion, and moving the fifth portion away from the sixth portion.

According to one embodiment, the hydraulic operation device comprises at least one fourth hydraulic actuator, directly or indirectly fixated to a seventh and eight portion of the first member. The hydraulic actuator being configured to operate the first member for at least one of: moving the seventh portion towards the eight portion, and moving the seventh portion away from the eight portion.

In one embodiment, the medical device comprises at least one hydraulic conduit for conducting a hydraulic fluid to at least one of the hydraulic actuators.

According to one embodiment, at least one of the first and second members comprises at least one recess or channel, and the hydraulic conduit may be configured to travel at least partially in the recess or channel.

According to one embodiment, at least one of the first and second members comprises at least one channel for conducting a hydraulic fluid. The medical device may in one embodiment further comprise at least one sheath. The at least one hydraulic actuator may be configured to travel through the sheath.

According to one embodiment, the sheath comprises elevated and lowered portions, such that the sheath can remain flexible even as fibrotic tissue overgrows the sheath.

According to one embodiment, the first member is flexible and/or elastic.

In one embodiment, the first member is more flexible than the second member.

In one embodiment, the first member is more elastic than the second member.

In one embodiment, the second member is substantially rigid. A major portion of the second member may be made from a material having a modulus of elasticity in the range 0.2 GPa-1000 GPa or in the range 1 GPa-400 GPa. The second member may have a modulus of elasticity in the range 0.2 GPa-1000 GPa or in the range 1 GPa-400 GPa.

At least one of the first and second member may be configured to be invaginated by the stomach wall using stomach-to-stomach sutures or staplers.

In one embodiment, at least one of the stomach engagers comprises at least one of: a fixation portion configured for fixation of the stomach engager to tissue of the stomach wall using at least one of sutures and staplers, and a tissue growth promoting structure such that the stomach engager can be fixated to the stomach wall by in-growth of fibrotic tissue.

A portion of the mechanical or hydraulic operation device may be placed in a remote unit configured to be placed at a remote location in the body of the patient. The medical device may further comprise a force transferring element configured to transfer force hydraulically or mechanically from the remote unit to the first member, for operating the first member.

The portion of the operation device placed in the remote unit may comprise at least one electrical motor.

The portion of the operation device placed in the remote unit may further comprise a gear system configured to reduce the speed and increase the force of the movement generated by the electrical motor.

In one embodiment, the portion of the operation device placed in the remote unit comprises at least one pulley for propelling the flexible element.

In one embodiment, the portion of the operation device placed in the remote unit may comprise at least one hydraulic pump.

The remote unit may comprise a hydraulic reservoir for holding a hydraulic fluid. The remote unit comprises an injection port for at least one of injecting and removing hydraulic fluid from the medical device.

The remote unit may further comprise an energy storage unit for directly or indirectly energizing the medical device. The remote unit may further comprise a controller for controlling the operation device. The controller may comprise a wireless transceiver for communicating wirelessly with a source external to the body of the patient.

In one embodiment, the medical device further comprising at least one sensor configured to sense a physical parameter of the medical device system.

In one embodiment, the sensor is a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

A medical device for actively stretching a stomach wall of a patient for creating a sensation of satiety is provided. The medical device being implantable and comprising a first member configured to be fixated to a first portion of the stomach wall, a second member configured to be fixated to a second portion of the stomach wall, and an operation device for operating the second member to displace the second member in a first direction relative to the first member, for stretching the stomach wall between the first and second portion. The medical device further comprises an acoustic sensor configured to sense at least one sound related to the patient swallowing, and a controller configured to receive a signal from the acoustic sensor, and control the operation device on the basis of the signal received from the acoustic sensor.

According to one embodiment, the acoustic sensor comprises at least one microphone.

According to one embodiment, the acoustic sensor comprises an implantable acoustic sensor.

According to one embodiment, the controller is configured to receive an acoustic sensor signal from an acoustic sensor being placed external to the body of the patient.

The sensor may further be configured to sense at least one parameter related to a functional status of the medical device, which may be a at least one parameter related to the operation of the operation device. In embodiments in which the operation device comprises an electrical motor, the sensor may be configured to sense at least one parameter related to the operation of the electrical motor.

A method of detecting that a patient swallows is further provided. The method comprises receiving a wireless signal from an implanted acoustic sensor configured to sense at least one sound related to the patient swallowing.

A method of implanting s medical device for actively stretching a stomach wall of a patient for creating a sensation of satiety is further provided. The method comprising fixating the medical device to the stomach wall of the patient, and implanting at least one acoustic sensor configured to sense at least one sound related to the patient swallowing.

The controller may comprise at least one unit having a sleep mode and an active mode, and the unit consumes less energy in the sleep mode than in the active mode. The unit is configured to switch from the sleep mode to the active mode on the basis of at least one signal from the acoustic sensor. The unit could for example be a DSP (Digital Signal Processor), another type of processor or a wake-up circuit of the controller, which in turn activates the functions of the controller. The unit may be configured to switch from the sleep mode to the active mode on the basis of a signal from the acoustic sensor related to the patient swallowing a number of times and/or on the basis of a signal from the acoustic sensor related to the patient swallowing a number of times during a time period. The number of times the patient swallows and the time could be counted/measured and compared with a pre-set or moving threshold value. The controller could further comprise at least one filtering unit configured to filter acoustic signals related to at least one of: speech, the swallowing of saliva and chewing. The filter could be a digital filter implemented as hardware or software in the controller and could have the filter characteristics of a high, low or bandpass filter.

A medical device for actively stretching a stomach wall of a patient for creating a sensation of satiety is provided. The medical device being implantable and comprising a first member configured to be fixated to a first portion of the stomach wall, a second member configured to be fixated to a second portion of the stomach wall, and an operation device for operating the second member to displace the second member in a first direction relative to the first member, for stretching the stomach wall between the first and second portion. The medical device further comprises a sensor configured to sense at least one parameter related to the patient swallowing, and be fixated to a structure of the body comprising bone. The medical device further comprises a controller configured to receive a signal from the sensor, and control the operation device on the basis of the signal received from the sensor.

According to one embodiment, the sensor comprises at least one acoustic sensor, which may be an acoustic sensor comprising at least one microphone.

According to one embodiment, the sensor comprises at least one accelerometer.

According to one embodiment, the sensor comprises at least one piezo-electric or piezo-resistive sensor.

According to one embodiment, the sensor comprises a strain gauge.

According to one embodiment, the sensor is configured to be fixated to at least one of: a sternum, and at least one rib of the patient.

The sensor configured to be fixated to the sternum or the at least one rib of the patient may be configured to be fixated to the sternum or the at least one rib of the patient, on the anterior side thereof.

The sensor configured to be fixated to the sternum or the at least one rib of the patient may be configured to be fixated to the sternum or the at least one rib of the patient, on the posterior side thereof.

The sensor may comprise an implantable sensor.

According to one embodiment, the sensor is configured to be fixated to the structure of the body comprising bone, on the outside of the skin of the patient.

According to one embodiment, the controller is configured to receive a sensor signal from a sensor being placed external to the body of the patient.

The sensor may be configured to sense at least one parameter related to a functional status of the medical device.

According to one embodiment, the sensor is configured to sense at least one parameter related to the operation of the operation device.

The controller may comprise at least one unit having a sleep mode and an active mode, and the unit consumes less energy in the sleep mode than in the active mode. The unit is configured to switch from the sleep mode to the active mode on the basis of at least one signal from the sensor. The unit could for example be a DSP (Digital Signal Processor), another type of processor or a wake-up circuit of the controller, which in turn activates the functions of the controller. The unit may be configured to switch from the sleep mode to the active mode on the basis of a signal from the sensor related to the patient swallowing a number of times and/or on the basis of a signal from the sensor related to the patient swallowing a number of times during a time period. The number of times the patient swallows and the time could be counted/measured and compared with a pre-set or moving threshold value. The controller could further comprise at least one filtering unit configured to filter acoustic signals related to at least one of: speech, the swallowing of saliva and chewing. The filter could be a digital filter implemented as hardware or software in the controller and could have the filter characteristics of a high, low or bandpass filter.

A method of detecting that a patient swallows is further provided. The method comprises receiving a wireless signal from an implanted sensor fixated to a structure of the body comprising bone and being configured to sense at least one sound related to the patient swallowing.

A method of implanting s medical device for actively stretching a stomach wall of a patient for creating a sensation of satiety is further provided. The method comprises fixating the medical device to the stomach wall of the patient, and fixating at least one sensor configured to sense at least one parameter related to the patient swallowing to a structure of the body comprising bone.

A medical device for actively stretching a stomach wall of a patient for creating a sensation of satiety is provided. The medical device being implantable and comprising a first member comprising a first stomach engager configured to engage a first portion of the stomach wall, and a second stomach engager portion configured to engage a second portion of the stomach wall. The medical device further comprises a first stomach engager configured to engage a third portion of the stomach wall, and a second stomach engager portion configured to engage a fourth portion of the stomach wall. The medical device further comprises a main portion, wherein the first and second members are operably connected to the main portion. The medical device further comprises an operation device comprising an actuator, wherein the operation device is configured to displace the actuator, such that the actuator is configured to: in a first state, operate the first member for displacing the first stomach engager of the first member in relation to the second stomach engager of the first member, for stretching the stomach wall, and in a second state, operate the second member for displacing the first stomach engager of the second member in relation to the second stomach engager of the second member, for stretching the stomach wall.

According to one embodiment, the actuator is configured to in a first state, operate the first member for displacing the first stomach engager of the first member in away from the second stomach engager of the first member, for stretching a stomach wall portion between the first and second stomach engagers, and in a second state, operate the second member for displacing the first stomach engager of the second member in away from the second stomach engager of the second member, for stretching a stomach wall portion between the first and second stomach engagers.

According to one embodiment, the actuator is configured to: in a first state, operate the first member for displacing the first stomach engager of the first member towards the second stomach engager of the first member, for at least stretching a stomach wall portion between the first and second members, and in a second state, operate the second member for displacing the first stomach engager of the second member towards the second stomach engager of the second member, for at least stretching a stomach wall portion between the first and second members.

The first and second stomach engagers may be pivotably fixated to the main portion, and the first and second stomach engagers of the first member may be pivotably fixated to the main portion at a common point of pivot.

According to one embodiment, the first member is scissor-like and the first and second stomach engagers of the first member are operable in a scissors-like manner, such that the first and second stomach engagers can be displaced towards each other and away from each other by the operation of the handle-like portion of the scissor-like first member.

According to one embodiment, the operation device is configured to displace the actuator by a rotational movement.

According to one embodiment, the operation device is configured to displace the actuator by a combination of linear and rotational movement.

The actuator may in a first state be engaged with the first member and in a second state be engaged with the second member.

According to one embodiment, the operation device is configured to disengage the actuator from the first member by linear displacement of the actuator.

The medical device may further comprise a third member comprising: a first stomach engager configured to engage a fifth portion of the stomach wall, and a second stomach engager portion configured to engage a sixth portion of the stomach wall. The operation device may be configured to displace the actuator, such that the actuator is configured to, in a third state, operate the third member for displacing the first stomach engager of the third member in relation to the second stomach engager of the third member, for stretching the stomach wall.

The medical device may further comprise a fourth member comprising: a first stomach engager configured to engage a seventh portion of the stomach wall, and a second stomach engager portion configured to engage an eight portion of the stomach wall. The operation device may be configured to displace the actuator, such that the actuator is configured to, in a fourth state, operate the fourth member for displacing the first stomach engager of the fourth member in relation to the second stomach engager of the fourth member, for stretching the stomach wall.

The operation device may comprise a hydraulic operation device and the actuator comprise a hydraulic actuator, and the hydraulic actuator may comprise at least one hydraulic cylinder.

According to one embodiment, the hydraulic actuator comprises at least one flexible member comprising elevated and lowered portions.

According to one embodiment, the flexible member comprises at least one bellows.

According to one embodiment, the elevated and lowered portions are configured to enable the hydraulic actuator to be operable when fibrotic tissue is covering the elevated and lowered portions.

According to one embodiment, the operation device comprises an implantable pump configured to at least one of: provide hydraulic fluid for displacing the actuator by a rotational movement, provide hydraulic fluid for displacing the actuator by a linear movement, and provide hydraulic fluid for operating the actuator for displacing the first stomach engager in relation to the second stomach engager. The implantable pump may be configured to be positioned at a remote location in relation to the main portion.

According to one embodiment, the operation device is operable by pressurized hydraulic fluid in a first direction and by an elastic element in an opposite direction.

According to one embodiment, the actuator is operable by pressurized hydraulic fluid in a first direction and by an elastic element in an opposite direction.

The operation device may further comprise a valve connected to the implantable pump.

The valve may be configured to: in a first state direct hydraulic fluid for displacing the actuator, and in a second state direct hydraulic fluid for operating the actuator for displacing the first stomach engager in relation to the second stomach engager.

According to one embodiment, the operation device comprises a mechanical operation device and the actuator comprises a mechanical actuator. The mechanical actuator may comprise at least one electrical motor or solenoid. The at least one electrical motor or solenoid may be configured to be positioned at a remote location in relation to the main portion.

According to one embodiment, the mechanical actuator may comprise at least one flexible member comprising elevated and lowered portions and the elevated and lowered portions may be configured to enable the mechanical actuator to be operable when fibrotic tissue is covering the elevated and lowered portions.

According to one embodiment, the electrical motor or solenoid is configured to at least one of: displace the actuator by a rotational movement, displace the actuator by a linear movement, and operate the actuator for displacing the first stomach engager in relation to the second stomach engager.

The stomach engagers may be configured to be invaginated by the stomach wall using stomach-to-stomach sutures or staplers.

According to one embodiment, the stomach engagers comprise at least one of: a fixation portion configured for fixation of the stomach engagers to tissue of the stomach wall using at least one of sutures and staplers, and a tissue growth promoting structure such that the stomach engagers can be fixated to the stomach wall by in-growth of fibrotic tissue.

The medical device may further comprise an energy storage unit for directly or indirectly energizing the medical device and/or a controller for controlling the operation device.

The controller may comprise a wireless transceiver for communicating wirelessly with a source external to the body of the patient.

According to one embodiment, the medical device further comprises at least one sensor, and the controller may be configured for receiving sensor input from the at least one sensor.

According to one embodiment, the sensor is a sensor configured to sense a physical parameter of the medical device system.

According to one embodiment, the sensor is a sensor configured to sense at least one of: a temperature of the medical device system, a parameter related to the power consumption of the medical device system, a parameter related to strain in the medical device system, a parameter related to a status of the energy storage unit, or a parameter related to the wireless transfer of energy from a source external to the body of the patient.

According to one embodiment, the sensor may be a sensor configured to sense a physiological parameter of the patient.

According to one embodiment, the sensor is a sensor configured to sense at least one of: a parameter related to the patient swallowing, a local temperature, a systemic temperature, blood saturation, blood oxygenation, blood pressure, a parameter related to an ischemia marker, or pH.

According to one embodiment, the sensor configured to sense a parameter related to the patient swallowing comprises at least one of: a motility sensor, a acoustic sensor, an optical sensor, or a strain sensor.

According to one embodiment, the sensor configured to sense pH is configured to sense the acidity in the stomach.

The controller may be configured to transmit information based on sensor input to a source external to the body of the patient.

An implantable operation device for operating a body engaging portion of the implantable medical device according to any of the embodiments herein is further provided, which may be one of the members described herein. The implantable operation device comprising an electrical machine for transforming electrical energy to mechanical force, a flexible shaft for transferring the mechanical force to a body engaging portion, and a pre-tensioning device for creating a pre-tension in the flexible shaft.

The pre-tension eliminates slack end e.g. ensures that the flexible shaft remain threaded onto pulleys or the spools/drums at all time.

The pre-tensioning device may comprise a flexible element, which may comprise at least one of a spring and an elastic material.

The flexible shaft may be configured for transferring a linear force.

The flexible shaft may be a Bowden cable.

According to one embodiment, the electrical machine comprises an electrical motor or solenoid and the implantable operation device may further comprise a transmission for transforming the mechanical force created by electrical transforming device from a rotating mechanical force to a linear mechanical force. The transmission may be configured for transforming the mechanical force created by electrical transforming device from a force having a first speed and a first strength to a force having a second lower speed and a second higher strength.

An implantable system is further provided comprising the implantable operation device according to any one of the preceding claims, and a body engaging portion configured to exert a force on a body portion of the patient, which may be a body engaging portion configured to engage the stomach wall of the patient for stretching the stomach wall such that satiety is created.

An implantable operation device for operating a body engaging portion of the implantable medical device according to any of the embodiments herein is further provided, which may be one of the members described herein. The implantable operation device comprising an electrical machine for transforming electrical energy to mechanical force, a transmission for transforming a mechanical force created by electrical transforming device from a force having a first speed and a first strength to a force having a second lower speed and a second higher strength. The transmission comprises a first and second pulley, a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.

According to one embodiment, the transmission comprises a gun tackle.

According to one embodiment, the transmission comprises a luff or watch tackle.

According to one embodiment, the transmission comprises a double tackle.

According to one embodiment, the transmission comprises a gyn tackle.

The implantable operation device may further comprise a third pulley, and the third pully may be connected to the electrical machine and the flexible element may be configured to be placed around the third pulley such that operation of the electrical machine pulls the flexible element.

According to one embodiment, the flexible element is, or transitions into, a flexible shaft for transferring the mechanical force to a body engaging portion.

The flexible shaft may be configured for transferring a linear force. The flexible shaft may be a Bowden cable.

According to one embodiment, the implantable operation device further comprising a pre-tensioning device for creating a pre-tension in the flexible element, which may be a pre-tensioning device comprising a flexible element which may comprise at least one of a spring and an elastic material.

According to one embodiment, the electrical machine comprises an electrical motor or solenoid. The electrical machine may be connected to a transmission for transforming the mechanical force created by electrical transforming device from a rotating mechanical force to a linear mechanical force.

According to one embodiment, the transmission may be configured for transforming the mechanical force created by the electrical machine from a force having a first speed and a first strength to a force having a second lower speed and a second higher strength.

An implantable system is further provided comprising the implantable operation device according to any one of the embodiments herein, and an implantable element configured to exert a force on a body portion of the patient.

An external device configured for communication with the implantable medical device according to any of the embodiments herein, when implanted in a patient, is further provided. The external device comprises at least one first wireless transceiver configured for communication with the implantable medical device using a first network protocol, for determining a distance between the external device and the implantable medical device, and at least one second wireless transceiver configured for communication with the implantable medical device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of: powering an energy consuming component of the implantable medical device, and charging an implantable energy storage unit.

According to one embodiment, the second network protocol is a standard network protocol.

According to one embodiment, the second wireless transceiver comprises a Bluetooth transceiver.

According to one embodiment, the external device is further configured to communicate with a second external device using the at least one wireless transceiver.

According to one embodiment, the external device is configured for determining a distance between the external device and the implantable medical device by determining the RSSI.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, a communication range of the first network protocol is less than a communication range of the second network protocol.

According to one embodiment, a frequency band of the first network protocol differs from a frequency band of the second network protocol.

According to one embodiment, the external device is configured to authenticate the implantable medical device if the determined distance between the external device and the implantable medical device is less than a predetermined threshold value. The external device may be configured to allow the transfer of data between the external device and the implantable medical device after the implantable medical device has been authenticated.

According to one embodiment, the external device is a wearable external device.

According to one embodiment, the external device is a handset.

An implantable medical device configured for communication with an external device according to one of the embodiments herein is further provided. The implantable medical device comprising at least one first wireless transceiver configured for communication with the external device using a first network protocol, for determining a distance between the external device and the implantable medical device, and at least one second wireless transceiver configured for communication with the external device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of: powering an energy consuming component of the implantable medical device, and charging an implantable energy storage unit.

According to one embodiment, the second network protocol is a standard network protocol.

According to one embodiment, the second wireless transceiver comprises a Bluetooth transceiver.

According to one embodiment, the implantable medical device is further configured to communicate with a second external device using said at least one wireless transceiver.

According to one embodiment, the implantable medical device is configured for determining a distance between the external device and the implantable medical device by determining the RSSI.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

The communication range of the first network protocol may be less than the communication range of the second network protocol.

The frequency band of the first network protocol may differ from a frequency band of the second network protocol.

According to one embodiment, the implantable medical device is configured to authenticate the external device if the determined distance between the external device and the implantable medical device is less than a predetermined threshold value.

According to one embodiment, the implantable medical device may be configured to allow the transfer of data between the implantable medical device and the external device after the external device has been authenticated.

An external device configured for communication with an implantable medical device according to any one of the embodiments disclosed herein is further provided. The external device comprising a wireless communication unit configured for wireless transmission of control commands to the implantable medical device and configured for wireless communication with a display device, and a computing unit configured for running a control software for creating the control commands for the operation of the implantable medical device. The computing unit may be configured to transmit a control interface to a display device configured to display the control interface to a user, receive user input from the display device, and transform the user input into the control commands for wireless transmission to the implantable medical device.

In one embodiment, the wireless communication unit comprises a wireless transceiver for wireless transmission of control commands to the implantable medical device, and wireless transmission of the control interface to the display device.

According to one embodiment, the wireless communication unit comprises a first wireless transceiver for wireless transmission of control commands to the implantable medical device, and a second wireless transceiver for wireless transmission of the control interface to the display device.

The wireless communication unit may in one embodiment be configured for wireless communication with the display device using a standard network protocol.

In one embodiment, the wireless communication unit is configured for wireless communication with the implantable medical device using a proprietary network protocol.

The wireless communication unit may comprise a Bluetooth transceiver, which may be comprised in one of the first and second wireless transceiver.

According to one embodiment, the wireless communication unit comprises a UWB transceiver, which may be comprised in one of the first and second wireless transceiver.

The wireless communication unit may comprise at least one first wireless transceiver configured for communication with the implantable medical device using a first network protocol, for determining a distance between the external device and the implantable medical device, and at least one second wireless transceiver configured for communication with the implantable medical device using a second network protocol, for transferring data between the external device and the implantable medical device.

According to one embodiment, the first wireless transceiver is configured for transcutaneous energy transfer for at least one of: powering an energy consuming component of the implantable medical device, and charging an implantable energy storage unit.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

The communication range of the first wireless transceiver may be less than a communication range of the second wireless transceiver

The frequency band of the first network protocol may differ from a frequency band of the second network protocol.

According to one embodiment, the external device is configured to authenticate the implantable medical device if a distance between the external device and the implantable medical device is less than a predetermined threshold value.

According to one embodiment, the external device is configured to be authenticated by the implantable medical device if a distance between the external device and the implantable medical device is less than a predetermined threshold value.

According to one embodiment, the external device is configured to authenticate the display device if a distance between the external device and the display device is less than a predetermined threshold value.

According to one embodiment, the external device is configured to be authenticated by the implantable medical device if a distance between the external device and the display device is less than a predetermined threshold value.

The external device may be configured to allow the transfer of data between the external device and the implantable medical device, and/or the external device and the display device, on the basis of the authentication.

According to one embodiment, the computing unit is configured to encrypt at least one of the control interface and the control commands.

A display device for communication with an external device for communication with an implantable medical device is further provided. The display device comprises a wireless communication unit configured for wirelessly receiving an implant control interface from the external device and configured for wirelessly transmitting implant control user input to the external device. The display device further comprising a display for displaying the received implant control interface, and an input device for receiving implant control input from the user.

According to one embodiment, the display device further comprises an auxiliary wireless communication unit configured to be disabled to enable at least one of: wirelessly receiving the implant control interface from the external device, and wirelessly transmitting implant control user input to the external device.

According to one embodiment, the wireless communication unit is configured for wireless communication with the external device using a standard network protocol.

According to one embodiment, the wireless communication unit is configured for wireless communication with the external device using a proprietary network protocol.

According to one embodiment, the wireless communication unit comprises a Bluetooth transceiver.

According to one embodiment, the wireless communication unit comprises a UWB transceiver.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

The communication range of the wireless communication unit of the display device may be less than a communication range of the auxiliary wireless communication unit.

According to one embodiment, the display device is configured to authenticate the external device if a distance between the display device and the external device is less than a predetermined threshold value.

According to one embodiment, the display device is configured to be authenticated by the external device if a distance between the display device and the external device is less than a predetermined threshold value.

According to one embodiment, the display device is configured to allow the transfer of data between the display device and the external device on the basis of the authentication.

The display device may be a wearable external device or a handset.

A communication system for enabling communication between a display device and an implantable medical device is further provided. The communication system comprising a display device, a server, and an external device. The display device comprises a wireless communication unit configured for wirelessly receiving an implant control interface from the server, the implant control interface being provided by the external device, the wireless communication unit further being configured for wirelessly transmitting implant control user input to the server, destined for the external device, a display for displaying the received implant control interface, and an input device for receiving implant control input from the user. The server of the communication system comprises: a wireless communication unit configured for wirelessly receiving an implant control interface from the external device and wirelessly transmitting the implant control interface to the display device, the wireless communication unit further being configured for wirelessly receiving implant control user input from the display device and wirelessly transmitting the implant control user input to the external device. The external device of the communication system comprises a wireless communication unit configured for wireless transmission of control commands to the implantable medical device and configured for wireless communication with the server, and a computing unit configured for: running a control software for creating the control commands for the operation of the implantable medical device, transmit a control interface to the server, destined for the display device, receive implant control user input generated at the display device, from the server, and transform the user input into the control commands for wireless transmission to the implantable medical device.

According to one embodiment, the computing unit of the communication system is configured to encrypt at least one of the control interface and the control commands.

According to one embodiment, the display device is configured to encrypt the user input.

According to one embodiment, the server is configured to encrypt at least one of the user input received from the display device and the control interface received from the external device.

According to one embodiment, the computing unit is configured to encrypt the control interface and the display device is configured to decrypt the encrypted control interface.

According to one embodiment, the server is configured to act as a router, transferring the encrypted control interface from the external device to the display device without decryption.

A display device for communication with an external device for communication with an implantable medical device is further provided. The display device comprising a wireless communication unit, a display, and an input device for receiving implant control input from the user. The display device is configured to run a first application for wireless communication with a server, and to run a second application for wireless communication with the external device for transmission of the implant control input to the external device for the communication with the implantable medical device, wherein the second application is configured to be accessed through the first application. The display device may comprise a first log-in function and a second log-in function, and wherein the first log-in function gives the user access to the first application and wherein the first and second log-in function in combination gives the user access to the second application.

According to one embodiment, the first log-in is a PIN-based log-in.

According to one embodiment, at least one of the first and second log-in is a log-in based on a biometric input or a hardware key.

According to one embodiment, the display device further comprises an auxiliary wireless communication unit, and the auxiliary wireless communication unit is configured to be disabled to enable wireless communication with the external device.

According to one embodiment, the display device is configured to wirelessly receive an implant control interface from the external device to be displayed on the display.

According to one embodiment of the display device, the wireless communication unit is configured for wireless communication with the external device using a standard network protocol.

According to one embodiment of the display device, the wireless communication unit is configured for wireless communication with the external device using a proprietary network protocol.

According to one embodiment of the display device, the wireless communication unit is configured for wireless communication with the external device using a first network protocol and with the server using a second network protocol.

According to one embodiment of the display device, the wireless communication unit is configured for wireless communication with the external device using a first frequency band and with the server using a second frequency band.

According to one embodiment of the display device, the wireless communication unit comprises a Bluetooth transceiver.

According to one embodiment of the display device, the wireless communication unit comprises a UWB transceiver.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the communication range of the wireless communication unit is less than a communication range of the auxiliary wireless communication unit.

According to one embodiment, the wireless communication unit comprises a first wireless transceiver for communication with the external device and a second wireless transceiver for communication with the server.

The second wireless transceiver may be configured to be disabled to enable wireless communication using the first wireless transceiver.

According to one embodiment, the display device is configured to authenticate the external device if a distance between the display device and the external device is less than a predetermined threshold value, and the display device is configured to be authenticated by the external device if a distance between the display device and the external device is less than a predetermined threshold value.

According to one embodiment, the display device is configured to allow the transfer of data between the display device and the external device on the basis of the authentication.

The display device may be a wearable external device or a handset.

According to one embodiment of the display device, the second application may be configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status, a temperature, a time, or an error.

According to one embodiment, the display device is configured to encrypt the user input.

According to one embodiment, the display is configured to encrypt the user input for decryption by the implantable medical device.

According to one embodiment, the display device is configured to decrypt the control interface received from the external device, for displaying the control interface on the display.

According to one embodiment, at least one of the first and second application is configured to receive data from an auxiliary external device and present the received data to the user.

At least one of the first and second application may be configured to receive data from an auxiliary external device comprising a scale for determining the weight of the user.

According to one embodiment, at least one of the first and second application may be configured to receive data related to the weight of the user from an auxiliary external device comprising a scale.

According to one embodiment, the display device is configured to: wirelessly transmit the data related to the weight of the user to the external device, or wirelessly transmit an instruction derived from the data related to the weight of the user, or wirelessly transmit an instruction derived from a combination of the data related to the weight of the user and the implant control input received from the user.

A communication system for enabling communication between a display device and an implantable medical device is further provided. The communication system comprises a display device, a server, and an external device. The display device comprises: a wireless communication unit configured for wirelessly receiving an implant control interface from the external device, the wireless communication unit further being configured for wirelessly transmitting implant control user input to the external device. The display device further comprises a display for displaying the received implant control interface, and an input device for receiving implant control input from the user, wherein the display device is configured to run a first application for wireless communication with the server, and to run a second application for wireless communication with the external device for transmission of the implant control input to the external device for the communication with the implantable medical device. The external device comprises a wireless communication unit configured for wireless transmission of control commands based on the implant control input to the implantable medical device and configured for wireless communication with the display device.

According to one embodiment, the display device comprises a first log-in function and a second log-in function, and wherein the first log-in function gives the user access to the first application and wherein the first and second log-in function in combination gives the user access to the second application.

The second application may be configured to receive data related to a parameter of the implanted medical device, and the second application may be configured to receive data related to a sensor value received from the implanted medical device.

According to one embodiment, the second application is configured to receive data related to a parameter related to at least one of: a battery status, a temperature, a time, or an error.

According to one embodiment, the display device is configured to encrypt the user input.

According to one embodiment, the display is configured to encrypt the user input for decryption by the implantable medical device.

According to one embodiment, the external device is configured to act as a router, transferring the encrypted user input from the display device to the implantable medical device without decryption.

According to one embodiment, the external device is configured to encrypt at least one of the control interface and the control commands.

According to one embodiment, the external device is configured to encrypt the control interface and wherein the display device is configured to decrypt the encrypted control interface.

A computer program product configured to run in a display device comprising a wireless communication unit, a display for displaying the received implant control interface, and an input device for receiving implant control input from a user is further provided. The computer program product comprising a first application for communication with a server, and a second application for communication with an external device for transmission of the implant control input to the external device for the communication with an implantable medical device, wherein the second application is configured to be accessed through the first application. The computer program product further comprises a first log-in function, and a second log-in function, wherein the first log-in function gives the user access to the first application and the first and second log-in function in combination gives the user access to the second application.

According to one embodiment of the computer program product, the second application is configured to receive data related to a parameter of the implanted medical device.

According to one embodiment of the computer program product, the second application is configured to receive data related to a sensor value received from the implanted medical device.

According to one embodiment of the computer program product, the second application is configured to receive data related to a parameter related to at least one of: a battery status, a temperature, a time, or an error.

A communication system for enabling communication between a display device, an external device, a server and an implantable medical device is further provided. The communication system comprising: a server, a display device, an external device, and an implantable medical device. The display device comprises: a wireless communication unit for wirelessly communicating with at least one of the external device and the server, a display, and an input device for receiving input from the user. The external device comprises: a wireless communication unit configured for wireless transmission of control commands to the implantable medical device and configured for wireless communication with at least one of the display device and the server. The server comprises: a wireless communication unit configured for wireless communication with at least one of the display device and the external device. The implantable medical device comprises: a wireless communication unit configured for wireless communication with the external device. The implantable medical device comprises an encryption unit which is configured to encrypt data destined for the server, transmit the data to the server via the external device, wherein the external device acts as a router transferring the data without full decryption, or the implantable medical device comprises an encryption unit and is configured to: encrypt data destined for the display device, transmit the data to the display device via the external device, wherein the external device acts as a router transferring the data without full decryption, or the server comprises an encryption unit and is configured to: encrypt data destined for the implantable medical device, transmit the data to the implantable medical device via the external device, wherein the external device acts as a router transferring the data without full decryption, or the server comprises an encryption unit and is configured to: encrypt data destined for the implantable medical device, transmit the data to the implantable medical device via the display device and the external device, wherein the display device and the external device acts as a router transferring the data without full decryption, or the display device comprises an encryption unit and is configured to: encrypt data destined for the implantable medical device, transmit the data to the implantable medical device via the external device, wherein the external device acts as a router transferring the data without full decryption, or the display device comprises an encryption unit and is configured to: encrypt data destined for the implantable medical device, transmit the data to the implantable medical device via the server and the external device, wherein the server and the external device acts as a router transferring the data without full decryption.

According to one embodiment, the display device is configured to wirelessly receive an implant control interface from the external device to be displayed on the display.

[000491] According to one embodiment of the communication system, at least two of: the wireless communication unit of the server, the wireless communication unit of the display device, the wireless communication unit of the external device, and the wireless communication unit of the implantable medical device—is configured for wireless communication using a standard network protocol.

According to one embodiment, the at least two of: the wireless communication unit of the server, the wireless communication unit of the display device, the wireless communication unit of the external device, and the wireless communication unit of the implantable medical device—is configured for wireless communication using a proprietary network protocol.

According to one embodiment, the wireless communication unit of the external device is configured to: use a first network protocol for communication with the implantable medical device and use a second network protocol for communication with the server, or use a first network protocol for communication with the implantable medical device and use a second network protocol for communication with the display device.

According to one embodiment, the wireless communication unit of the external device is configured to: use a first frequency band for communication with the implantable medical device and use a second frequency band for communication with the server, or use a first frequency band for communication with the implantable medical device and use a second frequency band for communication with the display device.

According to one embodiment, the wireless communication unit of the display device is configured to use a first network protocol for communication with the external device and use a second network protocol for communication with the server.

According to one embodiment, the wireless communication unit of the display device is configured to use a first frequency band for communication with the external device and use a second frequency band for communication with the server.

According to one embodiment, the wireless communication unit of the server is configured to use a first network protocol for communication with the external device and use a second network protocol for communication with the display device.

According to one embodiment, the wireless communication unit of the server is configured to use a first frequency band for communication with the external device and use a second frequency band for communication with the display device.

According to one embodiment, the wireless communication unit of at least one of the server, the display device, the external device, and the implantable medical device comprises a Bluetooth transceiver.

According to one embodiment, the wireless communication unit of at least one of the server, the display device, the external device, and the implantable medical device comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and a GSM type protocol.

According to one embodiment, the wireless communication unit of the external device comprises a first wireless transceiver for wireless communication with the implantable medical device, and a second wireless transceiver for wireless communication with the server, and wherein the second wireless transceiver has a longer effective range than the first wireless transceiver.

According to one embodiment, the wireless communication unit of the external device comprises a first wireless transceiver for wireless communication with the implantable medical device, and a second wireless transceiver for wireless communication with the display device, and wherein the second wireless transceiver has a longer effective range than the first wireless transceiver.

According to one embodiment, the wireless communication unit of the display device comprises a first wireless transceiver for wireless communication with the external device, and a second wireless transceiver for wireless communication with the server, and wherein the second wireless transceiver has a longer effective range than the first wireless transceiver.

According to one embodiment, the second wireless transceiver has an effective range being one of: 2 times, 4 times, 8 times 20 times, 50 times or 100 times longer than the first wireless transceiver.

According to one embodiment, the second wireless transceiver is configured to be disabled to enable wireless communication using the first wireless transceiver.

According to one embodiment, of the communication system, at least one of:
the display device is configured to authenticate the external device if a distance between the display device and the external device is less than a predetermined threshold value,
the display device is configured to be authenticated by the external device if a distance between the display device and the external device is less than a predetermined threshold value,
the display device is configured to authenticate the implantable medical device if a distance between the display device and the implantable medical device is less than a predetermined threshold value,
the display device is configured to be authenticated by the implantable medical device if a distance between the display device and the implantable medical device is less than a predetermined threshold value,
the external device is configured to authenticate the display device if a distance between the external device and the display device is less than a predetermined threshold value,
the external device is configured to be authenticated by the display device if a distance between the external device and the display device is less than a predetermined threshold value,
the external device is configured to authenticate the implantable medical device if a distance between the external device and the implantable medical device is less than a predetermined threshold value, and
the external device is configured to be authenticated by the implantable medical device if a distance between the external device and the implantable medical device is less than a predetermined threshold value.

According to one embodiment of the communication system, the display device may be configured to allow the transfer of data between the display device and the external device on the basis of the authentication.

According to one embodiment of the communication system, the external device is configured to allow the transfer of data between the display device and the external device on the basis of the authentication.

According to one embodiment of the communication system, the external device is configured to allow the transfer of data between the external device and the implantable medical device on the basis of the authentication.

According to one embodiment of the communication system, the display device is a wearable external device or a handset.

According to one embodiment of the communication system, the data encrypted by the implantable medical device is related to at least one of: a battery status, a temperature, a time, or an error.

A server for use in the communication system according to any one of the embodiments above is further provided. claims 1-24.

A display device for use in the communication system according to any one of the embodiments above is further provided.

An external device for use in the communication system according to any one of the embodiments above is further provided.

An implantable medical device for use in the communication system according to any one of the embodiments above is further provided.

Medical devices, designed to be implanted in a patient's body, are typically operated by means of electrical power. Such medical devices include electrical and mechanical stimulators, motors, pumps, etc, which are designed to support or stimulate various body functions. Electrical power can be supplied to such an implanted medical device from a likewise implanted battery or from an external energy source that can supply any needed amount of electrical power intermittently or continuously without requiring repeated surgical operations.

An implanted energy receiver or other implanted devices required for the operation of an implanted medical device must in some way be located in the patient's body in a secure and convenient way. It is often the case that the implanted device must be located close to the patient's skin in order to keep the distance between an external device, such as an energy transmitter, and the implanted device to a minimum. In practice, this means subcutaneous placement of the implanted device.

It is also often important that the implanted device is kept in a relatively fixed position so that for example energy transfer can be performed accurately.

According to one embodiment of the inventive concept, these and other objects are achieved in full, or at least in part, by an remote unit configured to be held in position by a tissue portion of a patient, the remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion, wherein the connecting portion is configured to connect the first portion to the second portion, wherein: the first, second, third and fourth planes are parallel to each other, the third cross-sectional area is smaller than the first, second and fourth cross-sectional areas, such that the first portion, second portion and connecting portion are prevented from travelling through the hole in the tissue portion in a direction perpendicular to the first, second and third planes, and the first portion is detachably connected to at least one of the connecting portion and the second portion.

In some embodiments, the connecting portion comprises a flange comprising the fourth cross-sectional area, such that the flange is prevented from travelling through the hole in the tissue portion in a direction perpendicular to the first, second and third planes.

In some embodiments, the flange protrudes in a direction parallel to the first, second, third and fourth planes, and perpendicular to a central extension of the connecting portion.

In some embodiments, the flange comprises the third surface configured to engage the first tissue surface of the first side of the tissue portion.

In some embodiments, the connecting portion comprises at least one protruding element comprising the fourth cross-sectional area, such that the at least one protruding element is prevented from travelling through the hole in the tissue portion, such that the second portion and the connecting portion can be held in position by the tissue portion of the patient also when the first portion is disconnected from the connecting portion.

In some embodiments, the at least one protruding element protrudes in a direction parallel to the first, second, third and fourth planes, and perpendicular to a central extension of the connecting portion.

In some embodiments, the at least one protruding element comprises the third surface configured to engage the first tissue surface of the first side of the tissue portion.

In some embodiments, the connecting portion comprises at least two protruding elements comprising the fourth cross-sectional area.

In some embodiments, the at least two protruding elements are symmetrically arranged about a central axis of the connecting portion.

In some embodiments, the at least two protruding elements are asymmetrically arranged about a central axis of the connecting portion.

In some embodiments, at least one of the first, second and third surfaces comprises at least one of ribs, barbs, hooks, a friction enhancing surface treatment, and a friction enhancing material, to facilitate the remote unit being held in position by the tissue portion.

In some embodiments, the connecting portion comprises a hollow portion.

In some embodiments, the hollow portion provides a passage between the first and second portions.

In some embodiments, the first portion is detachably connected to the connecting portion by at least one of a mechanical connection and a magnetic connection.

In some embodiments, the first portion is detachably connected to the connecting portion by at least one of threads and corresponding grooves, a screw, a self-locking element, a twist and lock fitting, and a spring-loaded locking mechanism.

In some embodiments, the at least one protruding element has a height in a direction perpendicular to the fourth plane being less than a height of the first portion in said direction.

In some embodiments, the at least one protruding element has a height in said direction perpendicular to the fourth plane being less than half of said height of the first portion in said direction.

In some embodiments, the at least one protruding element has a height in said direction perpendicular to the fourth plane being less than a quarter of said height of the first portion in said direction.

In some embodiments, the at least one protruding element has a height in said direction perpendicular to the fourth plane being less than a tenth of said height of the first portion in said direction.

In some embodiments, the at least one protruding element has a diameter in the fourth plane being one of: less than a diameter of the first portion in the first plane, equal to a diameter of the first portion in the first plane, and larger than a diameter of the first portion in the first plane.

In some embodiments, the at least one protruding element has a cross-sectional area in the fourth plane being one of: less than a cross-sectional area of the first portion in the first plane, equal to a cross-sectional area of the first portion in the first plane, and larger than a cross-sectional area of the first portion in the first plane.

In some embodiments, the at least one protruding element has a height in said direction perpendicular to the fourth plane being less than half of a height of the connecting portion in said direction.

In some embodiments, the at least one protruding element has a height in said direction perpendicular to the fourth plane being less than a quarter of said height of the connecting portion in said direction.

In some embodiments, the at least one protruding element has a height in said direction perpendicular to the fourth plane being less than a tenth of said height of the connecting portion in said direction.

According to one embodiment of the inventive concept, these and other objects are achieved in full, or at least in part, by an remote unit configured to be held in position by a tissue portion of a patient, the remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, and a connecting interface between the connecting portion and the second portion is excentric with respect to the second portion.

In some embodiments, the connecting interface between the connecting portion and the second portion is excentric, with respect to the second portion, in a first direction, but not in a second direction being perpendicular to the first direction.

In some embodiments, the connecting interface between the connecting portion and the second portion is excentric, with respect to the second portion, in a first direction and in a second direction being perpendicular to the first direction.

In some embodiments, the first direction and second direction are parallel to the second plane.

In some embodiments, the second portion has a first end and a second end opposing the first end, wherein the second portion has a length between the first and second end.

In some embodiments, the first end and second end are separated in a direction parallel to the second plane.

In some embodiments, the second portion is curved along the length.

In some embodiments, the second portion is curved in said first direction and said second direction being perpendicular to the first direction.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the second portion has said length in a direction being different to a central extension of the connecting portion.

In some embodiments, the second portion has a proximal region, an intermediate region, and a distal region.

In some embodiments, the proximal region extends from the first end to an interface between the connecting portion and the second portion, the intermediate region is defined by the connecting interface between the connecting portion and the second portion, and the distal region extends from the interface between the connecting portion and the second portion to the second end.

In some embodiments, the proximal region is shorter than the distal region with respect to the length of the second portion.

In some embodiments, the proximal region and the intermediate region together are shorter than the distal region with respect to the length of the second portion.

In some embodiments, the proximal region and the distal region comprises the second surface configured to engage the second surface of the second side of the tissue portion.

In some embodiments, the second portion has a length x and a width y along respective length and width directions being perpendicular to each other and substantially parallel to the second plane, wherein the connecting interface between the connecting portion and the second portion is contained within a region extending from x>0 to x<x/2 and/or y>0 to y<y/2, x and y and 0 being respective end points of the second portion along said length and width directions.

In some embodiments, the second portion is tapered from the first end to the second end.

In some embodiments, the second portion is tapered from each of the first end and second end towards the intermediate region of the second portion.

In some embodiments, the first portion has a maximum dimension being in the range of 10 to 40 mm, such as in the range of 10 to 30 mm, such as in the range of 15 to 25 mm.

In some embodiments, the first portion has a diameter being in the range of 10 to 40 mm, such as in the range of 10 to 30 mm, such as in the range of 15 to 25 mm.

In some embodiments, the connecting portion has a maximum dimension in the third plane in the range of 2 to 20 mm, such as in the range of 2 to 15 mm, such as in the range of 5 to 10 mm.

In some embodiments, the second portion has a maximum dimension being in the range of 30 to 90 mm, such as in the range of 30 to 70 mm, such as in the range of 35 to 60 mm.

In some embodiments, the first portion has one or more of a spherical shape, an ellipsoidal shape, a polyhedral shape, an elongated shape, and a flat disk shape.

In some embodiments, the connecting portion has one of an oval cross-section, an elongated cross-section, and a circular cross-section, in a plane parallel to the third plane.

In some embodiments, the distal region is configured to be directed downwards in a standing patient.

In some embodiments, the first portion comprises a proximal region extending from an first end to an interface between the connecting portion and the first portion, an intermediate region defined by an connecting interface between the connecting portion and the first portion, and a distal region extending from the interface between the connecting portion and the first portion to a second end of the first portion.

In some embodiments, the first portion has a first height, and the second portion has a second height, both heights being in a direction perpendicular to the first and second planes, wherein the first height is smaller than the second height.

In some embodiments, the first height is less than ⅔ of the second height, such as less than ½ of the second height, such as less than ⅓ of the second height.

In some embodiments, the second end of the second portion comprises connections for connecting to an implant being located in a caudal direction from a location of the remote unit in the patient.

In some embodiments, the first end of the second portion comprises connections for connecting to an implant being located in a cranial direction from a location of the remote unit in the patient.

According to one embodiment of the inventive concept, these and other objects are achieved in full, or at least in part, by an remote unit configured to be held in position by a tissue portion of a patient, the remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion has a third cross-sectional area in a third plane and is configured to connect the first portion to the second portion, wherein: the first, second and third planes are parallel to each other, the third cross-sectional area is smaller than the first and second cross-sectional areas, such that the first portion and second portion are prevented from travelling through the hole in the tissue portion in a direction perpendicular to the first, second and third planes, the first cross-sectional area has a first cross-sectional distance and a second cross-sectional distance, the first and second cross-sectional distances being perpendicular to each other and the first cross-sectional distance being longer than the second cross-sectional distance, the second cross-sectional area has a first cross-sectional distance and a second cross-sectional distance, the first and second cross-sectional distances being perpendicular to each other and the first cross-sectional distance being longer than the second cross-sectional distance, the first cross-sectional distance of the first cross-sectional area and the first cross-sectional distance of the second cross-sectional area are rotationally displaced in relation to each other with an angle exceeding 450 to facilitate insertion of the second portion through the hole in the tissue portion.

In some embodiments, the first cross-sectional distance of the first cross-sectional area and the first cross-sectional distance of the second cross-sectional area are rotationally displaced in relation to each other with an angle exceeding 600 to facilitate insertion of the second portion through the hole in the tissue portion.

In some embodiments, the first cross-sectional distance of the first cross-sectional area and the first cross-sectional distance of the second cross-sectional area are substantially perpendicular to each other to facilitate insertion of the second portion through the hole in the tissue portion.

In some embodiments, the first cross-sectional distance of the first cross-sectional area and the first cross-sectional distance of the second cross-sectional area are rotationally displaced in relation to each other with an angle exceeding 45° and being less than 135°.

In some embodiments, the cross-sectional area of the first portion is elongated.

In some embodiments, the cross-sectional area of the second portion is elongated.

In some embodiments, the connecting portion is connected eccentrically to the second portion.

In some embodiments, the first cross-sectional distance of the second portion is divided into a first, second and third equal length-portions, and wherein the connecting portion is connected to the second portion along the first length-portion of the first cross-sectional distance.

In some embodiments, the first cross-sectional area of the first portion is elongated.

In some embodiments, the second cross-sectional area of the second portion is elongated.

In some embodiments, the first portion comprises a first wireless energy receiver configured to receive energy transmitted wirelessly from an external wireless energy transmitter.

In some embodiments, the first portion comprises an internal wireless energy transmitter.

In some embodiments, the second portion comprises a second wireless energy receiver.

In some embodiments, the first portion comprises a first energy storage unit.

In some embodiments, the second portion comprises a second energy storage unit.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, and the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the remote unit further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the remote unit further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

According to one embodiment of the inventive concept, these and other objects are achieved in full, or at least in part, by an remote unit configured to be held in position by a tissue portion of a patient, the remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the remote unit further comprises at least one sensor for providing input to at least one of the first and second controller.

In some embodiments, the sensor is a sensor configured to sense a physical parameter of the remote unit.

In some embodiments, the sensor is a sensor configured to sense at least one of: a temperature of the remote unit or of a body engaging portion, a parameter related to the power consumption of the remote unit or of a body engaging portion, a parameter related to a status of at least one of the first and second energy storage unit, a parameter related to the wireless transfer of energy from a source external to the body of the patient, and a hydraulic pressure.

In some embodiments, the sensor is a sensor configured to sense a physiological parameter of the patient.

In some embodiments, the sensor is a sensor configured to sense at least one of: a parameter related to the patient swallowing, a local temperature, a systemic temperature, blood saturation, blood oxygenation, blood pressure, a parameter related to an ischemia marker, and pH.

In some embodiments, the sensor configured to sense a parameter related to the patient swallowing comprises at least one of: a motility sensor, a acoustic sensor, an optical sensor, and a strain sensor.

In some embodiments, the sensor configured to sense pH is configured to sense the acidity in the stomach.

In some embodiments, the controller is configured to transmit information based on sensor input to a device external to the body of the patient.

In some embodiments, the second portion comprises at least a portion of an operation device for operating an implantable body engaging portion.

In some embodiments, the second portion comprises at least one electrical motor.

In some embodiments, the second portion comprises a transmission configured to reduce the speed and increase the force of the movement generated by the electrical motor.

In some embodiments, the transmission is configured to transfer a week force with a high speed into a stronger force with lower speed.

In some embodiments, the transmission is configured to transfer a rotating force into a linear force.

In some embodiments, the transmission comprises a gear system.

In some embodiments, the second portion comprises a magnetic coupling for transferring mechanical work from the electrical motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

In some embodiments, the remote unit further comprises a capacitor connected to at least one of the first and second energy storage unit and connected to the electrical motor, wherein the capacitor is configured to: be charged by at least one of the first and second energy storage units, and provide the electrical motor with electrical power.

In some embodiments, at least one of the first and second portion comprises a sensation generator adapted to generate a sensation detectable by a sense of the patient.

In some embodiments, the second portion comprises a force transferring element configured to mechanically transfer force from the second portion to an implanted body engaging portion.

In some embodiments, the second portion comprises a force transferring element configured to hydraulically transfer force from the second portion to an implanted body engaging portion.

In some embodiments, the second portion comprises at least one lead for transferring electrical energy and/or information from the second portion to an implanted body engaging portion.

In some embodiments, the first portion comprises an injection port for injecting fluid into the first portion.

In some embodiments, the connecting portion comprises a conduit for transferring a fluid from the first portion to the second portion.

In some embodiments, the conduit is arranged to extend through the hollow portion of the connecting portion.

In some embodiments, the second portion comprises a first and a second chamber separated from each other, wherein the first chamber comprises a first liquid and the second chamber comprises a second liquid, and wherein the second liquid is a hydraulic liquid configured to transfer force to an implantable element configured to exert force on the body portion of the patient.

In some embodiments, a wall portion of the first chamber is resilient to allow an expansion of the first chamber.

In some embodiments, the second portion comprises a first hydraulic system in fluid connection with a first hydraulically operable implantable element configured to exert force on the body portion of the patient, and a second hydraulic system in fluid connection with a second hydraulically operable implantable element configured to exert force on the body portion of the patient, wherein the first and second hydraulically operable implantable elements are adjustable independently from each other.

In some embodiments, the first hydraulic system comprises a first hydraulic pump and the second hydraulic systems comprises a second hydraulic pump.

In some embodiments, each of the first and second hydraulic systems comprises a reservoir for holding hydraulic fluid.

In some embodiments, the implantable energized medical further comprises a first pressure sensor configured to sense a pressure in the first hydraulic system, and a second pressure sensor configured to sense a pressure in the second hydraulic system.

In some embodiments, the first surface is configured to engage the first tissue surface of the first side of the tissue portion.

In some embodiments, the first, second and third planes are parallel to a major extension plane of the tissue.

According to one embodiment of the inventive concept, these and other objects are achieved in full, or at least in part, by an remote unit configured to be held in position by a tissue portion of a patient, the remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, at least one of the first portion and the second portion comprises at least one coil embedded in a ceramic material, the at least one coil being configured for at least one of: receiving energy transmitted wirelessly, transmitting energy wirelessly, receiving wireless communication, and transmitting wireless communication.

In some embodiments, the first portion comprises a first wireless energy receiver configured to receive energy transmitted wirelessly from an external wireless energy transmitter.

In some embodiments, the first portion comprises a first wireless communication receiver.

In some embodiments, the first portion comprises a coil embedded in a ceramic material, hereinafter referred to as a first coil.

In some embodiments, the first wireless energy receiver comprises the first coil.

In some embodiments, the first wireless communication receiver comprises the first coil.

In some embodiments, the first portion comprises a distal end and a proximal end with respect to the connecting portion, along a direction perpendicular to the first plane.

In some embodiments, the first coil is arranged at the distal end of the first portion.

In some embodiments, the first portion comprises an internal wireless energy transmitter.

In some embodiments, the first portion comprises a first wireless communication transmitter.

In some embodiments, the first portion comprises a coil embedded in a ceramic material, hereinafter referred to as a second coil.

In some embodiments, the internal wireless energy transmitter comprises the second coil.

In some embodiments, the first wireless communication transmitter comprises the second coil.

In some embodiments, the second coil is arranged at the proximal end of the first portion.

In some embodiments, the first wireless energy receiver and the internal wireless energy transmitter comprises a single coil embedded in a ceramic material.

In some embodiments, the first wireless communication receiver and the first wireless communication transmitter comprises a single coil embedded in a ceramic material.

In some embodiments, the first wireless energy receiver, the internal wireless energy transmitter, the first wireless communication receiver, and the internal wireless communication transmitter comprises a single coil embedded in a ceramic material.

In some embodiments, the second portion comprises a second wireless energy receiver.

In some embodiments, the second portion comprises a coil embedded in a ceramic material, hereinafter referred to as a third coil, wherein the second wireless energy receiver comprises the third coil.

In some embodiments, the second portion comprises a distal end and a proximal end with respect to the connecting portion, along a direction perpendicular to the first plane.

In some embodiments, the third coil is arranged at the proximal end of the second portion.

In some embodiments, the first portion comprises a first energy storage unit.

In some embodiments, the second portion comprises a second energy storage unit.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first energy storage unit is configured to store less energy than the second energy storage unit, and configured to be charged faster than the second energy storage unit.

In some embodiments, the first energy storage unit has lower energy density than the second energy storage unit.

In some embodiments, the remote unit further comprises a housing configured to enclose at least the first portion, and wherein a first portion of the housing is made from titanium and a second portion of the housing is made from a ceramic material.

In some embodiments, the housing made from a ceramic material comprises the at least one coil embedded in the ceramic material.

In some embodiments, the remote unit further comprises a housing configured to enclose at least the second portion, and wherein a first portion of the housing is made from titanium and a second portion of the housing is made from a ceramic material.

In some embodiments, the portion of the housing made from a ceramic material comprises the at least one coil embedded in the ceramic material.

In some embodiments, the first, second and third planes are parallel to a major extension plane of the tissue.

In some embodiments, the connecting portion further comprises a fourt cross-sectional area in a fourth plane, wherein the fourt plane is parallel to the first, second and third planes, and wherein the third cross-sectional area is smaller than the fourth cross-sectional area.

In some embodiments, the connecting portion comprises a protruding element comprising the fourth cross-sectional area.

In some embodiments, the fourth plane is parallel to a major extension plane of the tissue.

In some embodiments, a connecting interface between the connecting portion and the second portion is excentric with respect to the second portion.

According to an embodiment of the inventive concept, an implantable device for exerting a force on a body portion of a patient is provided, wherein the implantable device comprises: an remote unit and an implantable element configured to exert a force on a body portion of the patient.

In some embodiments, the implantable element configured to exert a force on a body portion of the patient is an implantable hydraulic constriction device.

In some embodiments, the implantable hydraulic constriction device is configured for constricting a luminary organ of the patient.

In some embodiments, the implantable hydraulic constriction device comprises an implantable hydraulic constriction device for constricting an intestine of the patient.

In some embodiments, the implantable hydraulic constriction device comprises an implantable hydraulic constriction device for constricting a colon or rectum of the patient.

In some embodiments, the implantable hydraulic constriction device comprises an implantable hydraulic constriction device for constricting the intestine at a region of a stoma of the patient.

In some embodiments, the implantable hydraulic constriction device comprises an implantable hydraulic constriction device for constricting a blood vessel of the patient.

In some embodiments, the implantable hydraulic constriction device for constricting a blood vessel of the patient is configured to constrict the venous blood flow leading from an erectile tissue for promoting the engorgement of the erectile tissue.

In some embodiments, the implantable hydraulic constriction device comprises an implantable hydraulic constriction device for constricting a vas deference of the patient.

In some embodiments, the implantable element configured to exert a force on a body portion of the patient is an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the implantable element configured to exert a force on a body portion of the patient is an implantable element for actively stretching a stomach wall of the patient to create a feeling of satiety.

According to one embodiment, the electrical motor is a piezoelectric motor. Piezoelectric motors are advantageous in that they may have high precision, low power consumption, possible to be made small, lightweight, with high motion accuracy, and relatively immune to interference, such as electromagnetic interference. Piezoelectric motors can also be manufactured without magnetic and/or metallic parts.

According to one embodiment, the piezoelectric motor is a piezoelectric inchworm motor.

According to one embodiment, the piezoelectric motor is a piezoelectric inertial motor.

According to one embodiment, the piezoelectric motor is a piezoelectric walk-drive motor.

According to one embodiment, the piezoelectric motor is a linear piezoelectric motor, which may operate with at least one of: a speed in the range 1 mm/s to 10 mm/s, a stroke length in the range 4 mm-30 mm, and a force in the range 2 N-30 N.

According to one embodiment, the piezoelectric motor is a rotational piezoelectric motor, which may operate with at least one of: a rotational speed in the range 1 mrad/s-100 mrad/s, and a torque in the range 100 Nmm-900 Nmm.

According to one embodiment, the piezoelectric motor is a piezoelectric ultrasonic motor, which may be a traveling wave ultrasonic motor or a standing wave ultrasonic motor.

The piezoelectric ultrasonic motor may be a rotational piezoelectric ultrasonic motor which may operate with at least one of: a rotational speed in the range 10 mrad/s-10000 mrad/s, and a torque in the range 20 Nmm-450 Nmm.

The piezoelectric ultrasonic motor may be a linear piezoelectric ultrasonic motor, which may operate with at least one of: a speed in the range 4 mm/s-100 mm/s, and a force in the range 0,5N-30N.

According to one embodiment, the piezoelectric motor comprises at least one bimorph piezoelectric actuator.

The piezoelectric motor according to any one of the embodiments may be substantially non-magnetic.

The piezoelectric motor according to any one of the embodiments may be substantially non-metallic.

The piezoelectric motor according to any one of the embodiments may be a reversable piezoelectric motor.

According to one embodiment, a piezoelectric pump is provided comprising a first wall portion, a first diaphragm, a first chamber and a driving element. The first diaphragm and the first wall portion enclose the first chamber. The first wall portion comprises an inlet, configured to connect the first chamber to a first inlet reservoir, and an outlet, configured to connect the first chamber to a first outlet reservoir. The first diaphragm is configured to bend in response to operation of the driving element, and the driving element comprises a piezoelectric actuator, or is configured to be operated by a piezoelectric motor. Piezoelectric pumps are advantageous in that they are miniaturized and energy-efficient implantable devices. Piezoelectric pumps may precisely deliver fluid with a flow rate in the range 0.01 ml/min to 35 ml/min and a pressure in the range 0.2 kPa to 36 kPa.

According to one embodiment, a piezoelectric pump is provided wherein the inlet of the first wall portion comprises an inlet valve, and the outlet of the first wall portion comprises an outlet valve.

Any of the inlet valve of the first wall portion and outlet valve of the first wall portion may be a check valve, or an active valve. The check valve may be a ball valve. The inlet valve and outlet valve are useful to stabilize the flow rate in the first chamber.

According to one embodiment, the inlet of the first wall portion comprises an inlet static element, and the outlet of the first wall portion comprises an outlet static element, wherein any of the inlet static element and outlet static element is configured to act as a nozzle or a diffuser. Nozzles and diffusers are advantageous in that they are more resistant to wear and fatigue failure as compared to check valves and active valves.

According to one embodiment, the first diaphragm comprises a first movable wall portion.

The first movable wall portion may comprise elevated and lowered portions, wherein the elevated and lowered portions enable at least one of compression and expansion for moving the first movable wall portion. The first movable wall portion may comprise a substantially stiff portion. The first movable wall portion may comprises a bellows. The bellows may comprise metal. The bellows may comprise at least one of an oval cross-section, an elliptic cross-section and a circular cross-section. Metals are generally dense, which is advantageous as fluids do not diffuse through metals as easily. This reduces the risk that fluid diffuses from the first chamber or that fluids diffuse into the first chamber.

According to one embodiment, a piezoelectric pump is provided further comprising an auxiliary wall portion, and an auxiliary chamber sealed from the first chamber. The auxiliary wall portion and the first diaphragm enclose the auxiliary chamber. The sealing of the auxiliary chamber from first chamber is advantageous in that a component unsuitable to be in contact with the fluid in the first chamber may be hosted by the auxiliary chamber. Such a component may be the driving element.

According to one embodiment, a piezoelectric pump is provided further comprising an auxiliary wall portion, an auxiliary chamber sealed from the first chamber, and an auxiliary diaphragm configured to bend in the same direction as the first diaphragm in response to operation of the driving element. The auxiliary wall portion and the auxiliary diaphragm enclose the auxiliary chamber.

According to one embodiment, the auxiliary chamber is configured to be connected to a pressure adapter enabling variation of pressure in the auxiliary chamber. The pressure adapter may comprise an elastic portion having a surface area, and the elastic portion may be configured to maintain substantially the same surface area while enabling variation of pressure in the auxiliary chamber. This is advantageous in that a fibrotic tissue which at least partially covers the elastic portion may easily adapt to the elastic portion.

According to one embodiment, a piezoelectric pump is provided further comprising a second wall portion, a second diaphragm and a second chamber. The second diaphragm and the second wall portion enclose the second chamber. The second wall portion comprises an inlet, configured to connect the second chamber to a second inlet reservoir, and an outlet, configured to connect the second chamber to a second outlet reservoir. The second diaphragm is configured to bend in the same direction as the first diaphragm in response to operation of the driving element. The embodiment is advantageous in that the piezoelectric pump is configured to be operated in a double mode.

According to one embodiment, the inlet of the second wall portion comprises an inlet valve, and the outlet of the second wall portion comprises an outlet valve. Any of the inlet valve of the second wall portion and outlet valve of the second wall portion may be a check valve, or an active valve. The check valve may be a ball valve. The inlet valve and outlet valve are useful to stabilize the flow rate in the second chamber.

According to one embodiment, the inlet of the second wall portion comprises an inlet static element, and the outlet of the second wall portion comprises an outlet static element, wherein any of the inlet static element and outlet static element is configured to act as a nozzle or a diffuser. Nozzles and diffusers are advantageous in that they are more resistant to wear and fatigue failure as compared to check valves and active valves.

According to one embodiment, the piezoelectric pump comprises at least two portions connected in series, wherein each portion is a piezoelectric pump. The series connection allows for larger pressure compared to piezoelectric pumps in which fluid is transferred from an inlet to an outlet reservoir via a single chamber.

According to one embodiment, the piezoelectric pump comprises at least two portions connected in series, wherein each portion is a piezoelectric pump configured to be operated in a double mode. The series connection allows for larger pressure as compared to piezoelectric pumps configured to be operated in a double mode in which a first fluid is transferred from a first inlet reservoir to a first outlet reservoir via a single first chamber and a second fluid is transferred from a second inlet reservoir to a second outlet reservoir via a single second chamber.

According to one embodiment, the piezoelectric pump comprises at least two portions connected in parallel, wherein each portion is a piezoelectric pump. The parallel connection allows for a higher flow rate as compared to piezoelectric pumps in which the inlet reservoir is configured to be connected to a single inlet and the outlet reservoir is configured to be connected to a single outlet.

According to one embodiment, the piezoelectric pump comprises at least two portions connected in parallel, wherein each portion is a piezoelectric pump configured to be operated in a double mode. The parallel connection allows for a higher flow rate as compared to piezoelectric pumps configured to be operated in a double mode in which the first inlet reservoir and second inlet reservoir are configured to be connected to a single first inlet and single second inlet, respectively, and the first outlet reservoir and second outlet reservoir are configured to be connected to a single first outlet and single second outlet, respectively.

According to one embodiment, the medical device further comprises a controller configured to control the piezoelectric pump.

According to one embodiment, the medical device further comprises a sensor configured to measure a parameter of the piezoelectric pump, and a feedback unit. The sensor is further configured to transmit the measured parameter to the feedback unit. The feedback unit is configured to transmit a conditioning signal to the controller based on the measured parameter received from the sensor and based on a set value of the parameter. The controller is configured to adjust the control of the piezoelectric pump based on the conditioning signal received from the feedback unit in order for the measured parameter to achieve the set value.

[000726] According to one embodiment of the inventive concept, these and other objects are achieved in full, or at least in part, by a method of implanting an remote unit, the method comprising: placing a second portion of an remote unit between a peritoneum and a layer of muscular tissue of the abdominal wall, placing a first portion of the remote unit between the skin of the patient and a layer of muscular tissue of the abdominal wall, wherein the first and second portions are configured to be connected by a connecting portion extending through at least one layer of muscular tissue of the abdominal wall, placing a body engaging portion of the remote unit in connection with a tissue or an organ of the patient which is to be affected by the remote unit, and placing a transferring member, configured to transfer at least one of energy and force from the second portion to the body engaging portion, at least partially between a peritoneum and a layer of muscular tissue of the abdominal wall, such that at least ⅓ of the length of the transferring member is placed on the outside of the peritoneum.

In some embodiments, the transferring member is configured to transfer mechanical force from the second portion to the body engaging portion.

In some embodiments, the transferring member is configured to transfer hydraulic force from the second portion to the body engaging portion.

In some embodiments, the transferring member is configured to transfer electrical energy force from the second portion to the body engaging portion.

In some embodiments, the transferring member is configured to transfer data between the second portion and the body engaging portion.

In some embodiments, the step of placing the transferring member comprises placing the transferring member at least partially between the peritoneum and the layer of muscular tissue of the abdominal wall, such that at least ½ of the length of the transferring member is placed on the outside of the peritoneum of the patient.

In some embodiments, the step of placing the transferring member comprises placing the transferring member at least partially between the peritoneum and the layer of muscular tissue of the abdominal wall, such that at least ⅔ of the length of the transferring member is placed on the outside of the peritoneum of the patient.

In some embodiments, the step of placing the transferring member comprises placing the transferring member entirely outside of the peritoneum of the patient.

In some embodiments, the step of placing the transferring member comprises placing the transferring member such that it extends from the second portion to an area between the rib cage and the peritoneum of the patient, outside of the peritoneum.

In some embodiments, the step of placing the transferring member comprises placing the transferring member such that it extends from the second portion to an area between the stomach and the thoracic diaphragm of the patient.

In some embodiments, the step of placing the transferring member comprises placing the transferring member such that it extends from the second portion to the stomach of the patient.

In some embodiments, the step of placing the transferring member comprises placing the transferring member such that it extends from the second portion to the esophagus of the patient.

In some embodiments, the step of placing the transferring member comprises placing the transferring member such that it extends from the second portion to the retroperitoneal space.

In some embodiments, the step of placing the transferring member comprises placing the transferring member such that it extends from the second portion to an area of the kidneys.

In some embodiments, the step of placing the transferring member comprises placing the transferring member such that it extends from the second portion to the renal arteries.

In some embodiments, the step of placing the transferring member comprises placing the transferring member such that it extends from the second portion to the subperitoneal space, outside of the peritoneum.

In some embodiments, the step of placing the transferring member comprises placing the transferring member such that it extends from the second portion to the urinary bladder, outside of the peritoneum.

In some embodiments, the step of placing the transferring member comprises placing the transferring member such that it extends from the second portion to the urethra, outside of the peritoneum.

In some embodiments, the step of placing the second portion of the remote unit between the peritoneum and the layer of muscular tissue of the abdominal wall comprises placing the second portion between a first and second layer of muscular tissue of the abdominal wall.

In some embodiments, the step of placing the second portion comprises placing a second portion comprising an electrical motor.

In some embodiments, the step of placing the second portion comprises placing a second portion comprising a hydraulic pump.

In some embodiments, the step of placing the second portion comprises placing a second portion comprising an energy storage unit.

In some embodiments, the step of placing the second portion comprises placing a second portion comprising a receiver for receiving at least one of: energy and communication, wirelessly.

In some embodiments, the step of placing the first portion comprises placing a first portion comprising a transmitter for transmitting at least one of: energy and communication, wirelessly.

In some embodiments, the step of placing the second portion comprises placing a second portion comprising a controller involved in the control of the powered medical device.

In some embodiments, the second portion is elongated and has a length axis extending substantially in the direction of the elongation of the second portion, and wherein the step of placing the second portion comprises placing the second portion such that the length axis is substantially parallel with the cranial-caudal axis of the patient.

In some embodiments, the second portion is elongated and has a length axis extending substantially in the direction of the elongation of the second portion, and wherein the step of placing the second portion comprises placing the second portion such that the length axis is substantially perpendicular with the cranial-caudal axis of the patient.

In some embodiments, the second portion is elongated and has a length axis extending substantially in the direction of the elongation of the second portion, and wherein the step of placing the second portion comprises entering a hole in a layer of muscular tissue of the stomach wall in the direction of the length axis of the second portion and pivoting or angling the second portion after the hole has been entered.

In some embodiments, the step of placing the first portion of the remote unit between the skin of the patient and a layer of muscular tissue of the abdominal wall comprises placing the first portion in the subcutaneous tissue.

In some embodiments, the step of placing the first portion of the remote unit between the skin of the patient and a layer of muscular tissue of the abdominal wall comprises placing the first portion between a first and second layer of muscular tissue of the abdominal wall.

In some embodiments, the step of placing the first portion comprises placing a first portion comprising an energy storage unit.

In some embodiments, the step of placing the first portion comprises placing a first portion comprising a receiver for receiving at least one of: energy and communication, wirelessly.

In some embodiments, the step of placing the first portion comprises placing a first portion comprising a transmitter for transmitting at least one of: energy and communication, wirelessly.

In some embodiments, the step of placing the first portion comprises placing a first portion comprising a controller involved in the control of the powered medical device.

In some embodiments, the first portion is elongated and has a length axis extending substantially in the direction of the elongation of the first portion, and wherein the step of placing the first portion comprises placing the first portion such that the length axis is substantially parallel with the cranial-caudal axis of the patient.

In some embodiments, the first portion is elongated and has a length axis extending substantially in the direction of the elongation of the first portion, and wherein the step of placing the first portion comprises placing the first portion such that the length axis is substantially perpendicular with the cranial-caudal axis of the patient.

In some embodiments, the first portion is elongated and has a first portion length axis extending substantially in the direction of the elongation of the first portion, and the second portion is elongated and has a second portion length axis extending substantially in the direction of the elongation of the second portion, and wherein the step of placing the first and second portions comprises placing the first and second portions such that the first portion length axis and the second portion length axis are placed at an angle in relation to each other exceeding 30°.

In some embodiments, the step of placing the first and second portions comprises placing the first and second portions such that the first portion length axis and the second portion length axis are placed at an angle in relation to each other exceeding 45°.

In some embodiments, the method further comprises the step of placing the connecting portion through at least one layer of muscular tissue of the abdominal wall.

In some embodiments, the first portion, the second portion and the connecting portion are portions of a single unit.

In some embodiments, the method further comprises the step of connecting the first portion to the connecting portion, in situ.

In some embodiments, the method further comprises the step of connecting the second portion to the connecting portion, in situ.

In some embodiments, the method further comprises the step of connecting the transferring member to the first portion.

In some embodiments, the method further comprises the step of connecting the transferring member to the body engaging portion.

In some embodiments, the body engaging portion comprises a medical device for stretching the stomach wall such that a sensation of satiety is created.

In some embodiments, the body engaging portion comprises a constriction device configured to constrict a luminary organ of a patient.

In some embodiments, the body engaging portion comprises an implantable constriction device.

In some embodiments, the implantable constriction device comprises an implantable constriction device for constricting a luminary organ of the patient.

In some embodiments, the implantable constriction device comprises an implantable constriction device for constricting an intestine of the patient.

In some embodiments, the implantable constriction device comprises an implantable constriction device for constricting a colon or rectum of the patient.

In some embodiments, the implantable constriction device comprises an implantable constriction device for constricting the intestine at a region of a stoma of the patient.

In some embodiments, the implantable constriction device comprises an implantable constriction device for constricting a blood vessel of the patient.

In some embodiments, the implantable constriction device for constricting a blood vessel of the patient is configured to constrict the venous blood flow leading from an erectile tissue for promoting the engorgement of the erectile tissue.

In some embodiments, the implantable constriction device for constricting a blood vessel of the patient is configured to constrict the blood flow in the renal artery to affect the patients systemic blood pressure.

In some embodiments, the implantable constriction device comprises an implantable constriction device for constricting a vas deference of the patient.

In some embodiments, the body engaging portion comprises an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the body engaging comprises an element for electrically stimulating a tissue portion of a patient.

According to one embodiment of the inventive concept, these and other objects are achieved in full, or at least in part, by a kit for assembling an remote unit configured to be held in position by a tissue portion of a patient, the kit comprising: a group of one or more first portions, a group of one or more second portions, a group of one or more connecting portions, wherein at least one of said groups comprises at least two different types of said respective portions; wherein the remote unit is a modular device and, when assembled, comprises a selection, from said groups, of one first portion, one second portion, and one connecting portion, wherein: the first portion is configured to be placed on a first side of the tissue portion, the first portion having a first cross-sectional area in a first plane and comprising a first surface configured to face a first tissue surface of the first side of the tissue portion, the second portion is configured to be placed on a second side of the tissue portion, the second side opposing the first side, the second portion having a second cross-sectional area in a second plane and comprising a second surface configured to engage a second tissue surface of the second side of the tissue portion, and the connecting portion is configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, and the third cross-sectional area is smaller than the first and second cross-sectional areas, such that the first portion and second portion are prevented from travelling through the hole in the tissue portion in a direction perpendicular to the first, second and third planes.

In general, any of the embodiments of the remote unit disclosed herein may form part of such kit, and any features of such embodiments may be combined to form part of such kit.

In some embodiments, the group of one or more first portions comprises a first portion comprising a first energy storage unit.

In some embodiments, the group of one or more first portions comprises a first portion comprising a first wireless energy receiver unit for receiving energy transmitted wirelessly by an external wireless energy transmitter.

In some embodiments, the first energy storage unit is connected to the first wireless energy receiver, wherein the first wireless energy receiver is configured to receive energy transmitted wirelessly by the external wireless energy transmitter and store the received energy in the first energy storage unit.

In some embodiments, the first wireless energy receiver is configured to be physically connected to a second energy storage unit in the second portion.

In some embodiments, the group of one or more first portions comprises a first portion comprising an internal wireless energy transmitter.

In some embodiments, the group of one or more second portions comprises a second portion comprising a second wireless energy receiver configured to receive energy transmitted wirelessly by the internal wireless energy transmitter.

In some embodiments, the internal wireless energy transmitter is configured to transmit energy wirelessly to the second wireless energy receiver.

In some embodiments, the group of one or more second portions comprises a second portion comprising a second energy storage unit connected to the second wireless energy receiver.

In some embodiments, the second wireless energy receiver is configured to receive energy transmitted wirelessly by the internal wireless energy transmitter and store the received energy in the second energy storage unit.

In some embodiments, the group of one or more first portions comprises a first portion being formed as one integral unit with a connecting portion.

In some embodiments, the group of one or more second portions comprises a second portion being formed as one integral unit with a connecting portion.

In some embodiments, one of the group of one or more first, second or connecting portions comprises a first portion, second portion and connecting portion being formed as one integral unit.

In some embodiments, the group of one or more first portions comprises a first portion having a first height along a direction being perpendicular to the first plane, and a first portion having a second height along said direction being perpendicular to the first plane, wherein the second height is larger than the first height.

In some embodiments, the group of one or more first portions comprises a first portion having a first width and/or length along a direction being parallel to the first plane, and a first portion having a second width and/or length along said direction being parallel to the first plane, wherein the second width and/or length is larger than the first width and/or length.

In some embodiments, the group of one or more second portions comprises a second portion having a first height along a direction being perpendicular to the second plane, and a second portion having a second height along said direction being perpendicular to the second plane, wherein the second height is larger than the first height.

In some embodiments, the group of one or more second portions comprises a second portion having a first width and/or length along a direction being parallel to the second plane, and a second portion having a second width and/or length along said direction being parallel to the second plane, wherein the second width and/or length is larger than the first width and/or length.

In some embodiments, the group of one or more connecting portions comprises a connecting portion having a first height along a direction being perpendicular to the third plane, and a connecting portion having a second height along said direction being perpendicular to the third plane, wherein the second height is larger than the first height.

In some embodiments, the group of one or more connecting portions comprises a connecting portion having a first width and/or length along a direction being parallel to the third plane, and a connecting portion having a second width and/or length along said direction being parallel to the third plane, wherein the second width and/or length is larger than the first width and/or length.

In some embodiments, the group of one or more first portions comprises a first portion comprising an injection port for injecting fluid into the first portion.

In some embodiments, the group of one or more connecting portions comprises a connecting portion comprising a hydraulic fluid conduit for hydraulically connecting the first portion to the second portion.

In some embodiments, the group of one or more first portions comprises a first portion comprising a first controller comprising at least one processing unit.

In some embodiments, the group of one or more second portions comprises a second portion comprising a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the group of first portions comprises a first portion comprising a combined coil, wherein the combined coil is configured to receive wireless energy wirelessly from an external wireless energy transmitter, and transmit wireless energy wirelessly to the second wireless receiver of the second portion.

In some embodiments, at least one of the coils are embedded in a ceramic material.

In some embodiments, the group of one or more first portions comprises a first portion comprising a push button and/or a capacitive button for controlling a function of the remote unit.

The term “body tissue” referred to in the present disclosure may be one or several body tissue groups or layers in a patient, such as muscle tissue, connective tissue, bone, etc.

An external device configured for communication with an implantable medical device, when implanted in a patient, is provided. The external device comprises at least one first wireless transceiver configured for communication with the implantable medical device using a first network protocol, for determining a distance between the external device and the implantable medical device, and at least one second wireless transceiver configured for communication with the implantable medical device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of powering an energy consuming component of the implantable medical device and charging an implantable energy storage unit.

According to one embodiment, the second network protocol is a standard network protocol. The standard network protocol may be one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the second wireless transceiver comprises a Bluetooth transceiver.

According to one embodiment, the external device is further configured to communicate with a second external device using said at least one wireless transceiver.

According to one embodiment, the external device is configured for determining a distance between the external device and the implantable medical device by determining the RSSI.

According to one embodiment, a communication range of the first network protocol is less than a communication range of the second network protocol.

According to one embodiment, a frequency band of the first network protocol differs from a frequency band of the second network protocol.

According to one embodiment, the external device is configured to authenticate the implantable medical device if the determined distance between the external device and the implantable medical device is less than a predetermined threshold value.

According to one embodiment, the external device is configured to allow the transfer of data between the external device and the implantable medical device after the implantable medical device has been authenticated.

According to one embodiment, the external device is one from the list of: a wearable external device, and a handset.

An implantable medical device configured for communication with an external device is provided. The implantable medical device comprises at least one first wireless transceiver configured for communication with the external device using a first network protocol, for determining a distance between the external device and the implantable medical device, and at least one second wireless transceiver configured for communication with the external device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of: powering an energy consuming component of the implantable medical device, and charging an implantable energy storage unit.

According to one embodiment, the second network protocol is a standard network protocol, such as selected from the list of Radio Frequency type protocol, RFID type protocol, WLAN type protocol, Bluetooth type protocol, BLE type protocol, NFC type protocol, 3G/4G/5G type protocol, and GSM type protocol.

According to one embodiment, the second wireless transceiver comprises a Bluetooth transceiver.

According to one embodiment, the implantable medical device is further configured to communicate with a second external device using said at least one wireless transceiver.

According to one embodiment, the implantable medical device is configured for determining a distance between the external device and the implantable medical device by determining the RSSI.

According to one embodiment, a communication range of the first network protocol is less than a communication range of the second network protocol.

According to one embodiment, a frequency band of the first network protocol differs from a frequency band of the second network protocol.

According to one embodiment, the implantable medical device is configured to authenticate the external device if the determined distance between the external device and the implantable medical device is less than a predetermined threshold value.

According to one embodiment, the implantable medical device is configured to allow the transfer of data between the implantable medical device and the external device after the external device has been authenticated.

According to one embodiment, the implantable medical device comprises at least one of:
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries,
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created

reservoir,

an implant communicating with a database outside the body,

an implant able to be programmed from outside the body,

an implant able to be programmed from outside the body with a wireless signal,

an implant treating impotence,

an implant controlling the flow of eggs in the uterine tube,

an implant controlling the flow of sperms in the uterine tube,

an implant controlling the flow of sperms in the vas deferens,

an implant for hindering the transportation of the sperm in the vas deferens,

an implant treating osteoarthritis,

an implant performing a test of parameters inside the body,

an implant controlling specific treatment parameters from inside the body,

an implant controlling bodily parameters from inside the body,

an implant controlling the blood pressure,

an implant controlling the blood pressure by affecting the dilatation of the renal artery,

an implant controlling a drug treatment parameter,

an implant controlling a parameter in the blood,

an implant for adjusting or replacing any bone part of a body of the patient,

an implant replacing an organ of the patient or part of an organ of the patient or the function thereof,

a vascular treatment device,

an implant adapted to move fluid inside the body of the patient,

an implant configured to sense a parameter related to the patient swallowing,

an implant configured to exercise a muscle with electrical or mechanical stimulation,

an implant configured for emptying an intestine portion on command,

an operable implant configured to be invaginated in the stomach of the patient to reduce the volume of the stomach substantially more than the volume of the device,

an implant configured for emptying the urinary bladder from within the patient's body by compressing the bladder,

an implant configured for draining fluid from within the patient's body,

an implant configured for the active lubrication of a joint with an added lubrication fluid,

an implant configured for removing clots and particles from the patient's blood stream,

an implant configured for elongating or straightening a bone in the patient, to reduce scoliosis,

a device to stimulate the brain for a several position to a focused point,

an artificial stomach replacing the function of the natural stomach,

an implant configured for adjusting the position of a female's urinary tract or bladder neck,

an implant configured for stimulating the ampulla vas deference and creating temporary constriction.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallow solid food or is drinking fluid, wherein said food sensor is connected to the control unit of a medical device to cause an action to stretch the stomach after a determined amount of food intake.

A patient external device configured for communication with an implantable medical device, when implanted in a patient, is provided. The patient external device comprises a wireless communication unit configured for wireless transmission of control commands to the implantable medical device and configured for wireless communication with a patient display device, and a computing unit configured for running a control software for creating the control commands for the operation of the implantable medical device. The computing unit is configured to transmit a control interface as a remote display portal to a patient display device configured to display the control interface to a user, receive user input from the patient display device, and transform the user input into the control commands for wireless transmission to the implantable medical device.

According to one embodiment, the wireless communication unit comprises a wireless transceiver for wireless transmission of control commands to the implantable medical device, and wireless transmission of the control interface as the remote display portal to the patient display device.

According to one embodiment, the wireless communication unit comprises a first wireless transceiver for wireless transmission of control commands to the implantable medical device, and a second wireless transceiver for wireless transmission of the control interface to the patient display device.

According to one embodiment, the wireless communication unit is configured for wireless communication with the patient display device using a standard network protocol.

According to one embodiment, the wireless communication unit is configured for wireless communication with the implantable medical device using a proprietary network protocol.

According to one embodiment, the wireless communication unit comprises a Bluetooth transceiver.

According to one embodiment, at least one of the first and second wireless transceiver comprises a Bluetooth transceiver.

According to one embodiment, the wireless communication unit comprises a UWB transceiver.

According to one embodiment, at least one of the first and second wireless transceiver comprises a UWB transceiver.

According to one embodiment, the wireless communication unit comprises at least one first wireless transceiver configured for communication with the implantable medical device using a first network protocol, for determining a distance between the patient external device and the implantable medical device, and at least one second wireless transceiver configured for communication with the implantable medical device using a second network protocol, for transferring data between the patient external device and the implantable medical device.

According to one embodiment, the first wireless transceiver is configured for transcutaneous energy transfer for at least one of: powering an energy consuming component of the implantable medical device and charging an implantable energy storage unit.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, a communication range of the first wireless transceiver is less than a communication range of the second wireless transceiver.

According to one embodiment, at least one of:
the patient external device is configured to authenticate the implantable medical device if a distance between the patient external device and the implantable medical device is less than a predetermined threshold value,
the patient external device is configured to be authenticated by the implantable medical device if a distance between the patient external device and the implantable medical device is less than a predetermined threshold value,
the patient external device is configured to authenticate the patient display device if a distance between the patient external device and the patient display device is less than a predetermined threshold value, and
the patient external device is configured to be authenticated by the implantable medical device if a distance between the patient external device and the patient display device is less than a predetermined threshold value.

According to one embodiment, the patient external device is configured to allow the transfer of data between at least one of: the patient external device and the implantable medical device, and the patient external device and the patient display device, on the basis of the authentication.

According to one embodiment, the computing unit is configured to encrypt at least one of the control interface and the control commands.

According to one embodiment, the implantable medical device comprises at least one of:
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.

an implantable drug delivery device,

an implantable drug delivery device for injecting directly into a blood vessel and change the position of the injection site, all from within the patient's body,

an implantable drug delivery device for injecting potency enhancing drugs into an erectile tissue of the patient,

a hydraulic, mechanic, and/or electric constriction implant,

an operable volume filling device,

an operable gastric band,

an operable implant for stretching the stomach wall of the patient for creating satiety,

an implant configured to sense the frequency of the patient ingesting food,

an operable cosmetic implant,

an operable cosmetic implant for adjust the shape and/or size in the breast region of a patient,

an implant controlling medical device for the emptying of a urinary bladder,

an implant hindering urinary leakage,

an implant hindering anal incontinence,

an implant controlling the emptying of fecal matter,

an implant monitoring an aneurysm,

an implant for hindering the expansion of an aneurysm,

an implant lubricating a joint,

an implant for affecting the blood flow to an erectile tissue of the patient,

an implant for simulating the engorgement of an erectile tissue,

an implant with a reservoir for holding bodily fluids,

an implant storing and/or emptying a bodily reservoir or a surgically created

reservoir,

an implant communicating with a database outside the body,

an implant able to be programmed from outside the body,

an implant able to be programmed from outside the body with a wireless signal,

an implant treating impotence,

an implant controlling the flow of eggs in the uterine tube,

an implant controlling the flow of sperms in the uterine tube,

an implant controlling the flow of sperms in the vas deferens,

an implant for hindering the transportation of the sperm in the vas deferens,

an implant treating osteoarthritis,

an implant performing a test of parameters inside the body,

an implant controlling specific treatment parameters from inside the body,

an implant controlling bodily parameters from inside the body,

an implant controlling the blood pressure,

an implant controlling the blood pressure by affecting the dilatation of the renal artery,

an implant controlling a drug treatment parameter,

an implant controlling a parameter in the blood,

an implant for adjusting or replacing any bone part of a body of the patient,

an implant replacing an organ of the patient or part of an organ of the patient or the function thereof,

a vascular treatment device,

an implant adapted to move fluid inside the body of the patient,

an implant configured to sense a parameter related to the patient swallowing,

an implant configured to exercise a muscle with electrical or mechanical stimulation,

an implant configured for emptying an intestine portion on command,

an operable implant configured to be invaginated in the stomach of the patient to reduce the volume of the stomach substantially more than the volume of the device,

an implant configured for emptying the urinary bladder from within the patient's body by compressing the bladder,

an implant configured for draining fluid from within the patient's body,

an implant configured for the active lubrication of a joint with an added lubrication fluid,

an implant configured for removing clots and particles from the patient's blood stream,

an implant configured for elongating or straightening a bone in the patient, to reduce scoliosis,

a device to stimulate the brain for a several position to a focused point,

an artificial stomach replacing the function of the natural stomach,

an implant configured for adjusting the position of a female's urinary tract or bladder neck,

an implant configured for stimulating the ampulla vas deference and creating temporary constriction.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A patient display device for communication with a patient remote external device for communication with an implantable medical device is provided. The patient display device comprises a wireless communication unit configured for wirelessly receiving an implant control interface as a remote display portal from the patient remote external device and configured for wirelessly transmitting implant control user input to the patient remote external device, a display for displaying the received implant control interface, and an input device for receiving implant control input from the user.

According to one embodiment, the patient display device further comprises an auxiliary wireless communication unit. The auxiliary wireless communication unit is configured to be disabled to enable at least one of: wirelessly receiving the implant control interface as the remote display portal from the patient remote external device, and wirelessly transmitting implant control user input to the patient remote external device.

According to one embodiment, the wireless communication unit is configured for wireless communication with the patient remote external device using a standard network protocol. The standard network protocol may be one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the wireless communication unit is configured for wireless communication with the patient remote external device using a proprietary network protocol.

According to one embodiment, the wireless communication unit comprises a Bluetooth transceiver.

According to one embodiment, the wireless communication unit comprises a UWB transceiver.

According to one embodiment, a communication range of the wireless communication unit is less than a communication range of the auxiliary wireless communication unit.

According to one embodiment, the patient display device is configured to authenticate the patient remote external device if a distance between the patient display device and the patient remote external device is less than a predetermined threshold value, or to be authenticated by the patient remote external device if a distance between the patient display device and the patient remote external device is less than a predetermined threshold value.

According to one embodiment, the patient display device is configured to allow the transfer of data between the patient display device and the patient remote external device on the basis of the authentication.

According to one embodiment, the patient display device is a wearable external device or a handset.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A communication system for enabling communication between a patient display device and an implantable medical device, when implanted, is provided. The communication system comprises: a patient display device,

a server, and a patient remote external device. The patient display device comprises a wireless communication unit configured for wirelessly receiving an implant control interface as a remote display portal being provided by the patient remote external device. The wireless communication unit is further configured for wirelessly transmitting implant control user input to the server, destined for the patient remote external device. The system further comprises a display for displaying the received remote display portal, and an input device for receiving implant control input from the user, wherein the patient remote external device comprises a wireless communication unit configured for wireless transmission of control commands to the implantable medical device, and a computing unit. The computing unit is configured for running a control software for creating the control commands for the operation of the implantable medical device, transmitting a control interface to the patient display device, receiving implant control user input generated at the patient display device, from the server, and transforming the user input into the control commands for wireless transmission to the implantable medical device.

According to one embodiment, the computing unit is configured to encrypt at least one of the control interface and the control commands.

According to one embodiment, the patient display device is configured to encrypt the user input.

According to one embodiment, the server is configured to encrypt at least one of the user input received from the patient display device and the control interface received from the patient remote external device.

According to one embodiment, the computing unit is configured to encrypt the control interface and the patient display device is configured to decrypt the encrypted control interface.

According to one embodiment, the server is configured to act as a router, transferring the encrypted control interface from the patient remote external device to the patient display device without decryption.

According to one embodiment of the communication system or patient display device the implantable medical device comprises at least one of:

an external heart compression device,

an apparatus assisting the pump function of a heart of the patient,

an apparatus assisting the pump function comprising a turbine bump placed within a patient's blood vessel for assisting the pump function of the heart,

an operable artificial heart valve,

an operable artificial heart valve for increasing the blood flow to the coronary arteries.

an implantable drug delivery device,

an implantable drug delivery device for injecting directly into a blood vessel and change the position of the injection site, all from within the patient's body,

an implantable drug delivery device for injecting potency enhancing drugs into an erectile tissue of the patient,

a hydraulic, mechanic, and/or electric constriction implant,

an operable volume filling device,

an operable gastric band,

an operable implant for stretching the stomach wall of the patient for creating satiety,

an implant configured to sense the frequency of the patient ingesting food,

an operable cosmetic implant,

an operable cosmetic implant for adjust the shape and/or size in the breast region of a patient,

an implant controlling medical device for the emptying of a urinary bladder,

an implant hindering urinary leakage,

an implant hindering anal incontinence,

an implant controlling the emptying of fecal matter,

an implant monitoring an aneurysm,

an implant for hindering the expansion of an aneurysm,

an implant lubricating a joint,

an implant for affecting the blood flow to an erectile tissue of the patient,

an implant for simulating the engorgement of an erectile tissue,

an implant with a reservoir for holding bodily fluids,

an implant storing and/or emptying a bodily reservoir or a surgically created

reservoir,

an implant communicating with a database outside the body,

an implant able to be programmed from outside the body,

an implant able to be programmed from outside the body with a wireless signal,

an implant treating impotence,

an implant controlling the flow of eggs in the uterine tube,

an implant controlling the flow of sperms in the uterine tube,

an implant controlling the flow of sperms in the vas deferens,

an implant for hindering the transportation of the sperm in the vas deferens,

an implant treating osteoarthritis,

an implant performing a test of parameters inside the body,

an implant controlling specific treatment parameters from inside the body,

an implant controlling bodily parameters from inside the body,

an implant controlling the blood pressure,

an implant controlling the blood pressure by affecting the dilatation of the renal artery,

an implant controlling a drug treatment parameter,

an implant controlling a parameter in the blood,

an implant for adjusting or replacing any bone part of a body of the patient,

an implant replacing an organ of the patient or part of an organ of the patient or the function thereof,

a vascular treatment device,

an implant adapted to move fluid inside the body of the patient,

an implant configured to sense a parameter related to the patient swallowing,

an implant configured to exercise a muscle with electrical or mechanical stimulation,

an implant configured for emptying an intestine portion on command,

an operable implant configured to be invaginated in the stomach of the patient to reduce the volume of the stomach substantially more than the volume of the device,

an implant configured for emptying the urinary bladder from within the patient's body by compressing the bladder,

an implant configured for draining fluid from within the patient's body,

an implant configured for the active lubrication of a joint with an added lubrication fluid,

an implant configured for removing clots and particles from the patient's blood stream,

an implant configured for elongating or straightening a bone in the patient, to reduce scoliosis,

a device to stimulate the brain for a several position to a focused point,

an artificial stomach replacing the function of the natural stomach,

an implant configured for adjusting the position of a female's urinary tract or bladder neck,

an implant configured for stimulating the ampulla vas deference and creating temporary constriction.

According to one embodiment, the communication system further comprises a server. The server may comprise a wireless communication unit configured for wirelessly receiving an implant control interface received from the patient remote external device and wirelessly transmitting the implant control interface as a remote display portal to the patient display device. The wireless communication unit is further configured for wirelessly receiving implant control user input from a patient EID external device and wirelessly transmitting the implant control user input to the patient display device.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A patient display device for communication with a patient external device for communication with an implantable medical device, when implanted, is provided. The patient display device comprises a wireless communication unit, a display, and an input device for receiving implant control input from the user. The patient display device is configured to run a first application for wireless communication with a server and/or DDI, and run a second application for wireless communication with the patient external device for transmission of the implant control input to a remote display portal of the patient external device for the communication with the implantable medical device, wherein the second application is configured to be accessed through the first application. The patient display device comprises a first log-in function and a second log-in function, wherein the first log-in function gives the user access to the first application and wherein the first and second log-in function in combination gives the user access to the second application. The first log-in function may be configured to use at least one of a password, pin code, fingerprint, voice and face recognition. A second log-in function within the first application may be configured to use a private key from the user to authenticate, for a defined time period, a second hardware key of the patient external device.

According to one embodiment, the first log-in is a PIN-based log-in.

According to one embodiment, at least one of the first and second log-in is a log-in based on a biometric input or a hardware key.

According to one embodiment, the patient display device further comprises an auxiliary wireless communication unit, and wherein the auxiliary wireless communication unit is configured to be disabled to enable wireless communication with the patient external device.

According to one embodiment, the patient display device is configured to wirelessly receive an implant control interface as a remote display portal from the patient external device to be displayed on the display.

According to one embodiment, the wireless communication unit is configured for wireless communication with the patient external device using a standard network protocol.

According to one embodiment, the wireless communication unit is configured for wireless communication with the patient external device using a proprietary network protocol.

According to one embodiment, the wireless communication unit is configured for wireless communication with the patient external device using a first network protocol and with the server using a second network protocol.

According to one embodiment, the wireless communication unit is configured for wireless communication with the patient external device using a first frequency band and with the server using a second frequency band.

According to one embodiment, the wireless communication unit comprises a Bluetooth transceiver.

According to one embodiment, the wireless communication unit comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, a communication range of the wireless communication unit is less than a communication range of the auxiliary wireless communication unit.

According to one embodiment, the wireless communication unit comprises a first wireless transceiver for communication with the patient external device and a second wireless transceiver for communication with the server.

According to one embodiment, the second wireless transceiver is configured to be disabled to enable wireless communication using the first wireless transceiver.

According to one embodiment, the patient display device is configured to authenticate the patient external device if a distance between the patient display device and the patient external device is less than a predetermined threshold value, or to be authenticated by the patient external device if a distance between the patient display device and the patient external device is less than a predetermined threshold value.

According to one embodiment, the patient display device is configured to allow the transfer of data between the patient display device and the patient external device on the basis of the authentication.

According to one embodiment, the patient display device is a wearable external device or a handset.

According to one embodiment, the second application is configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status, a temperature, a time, and an error.

According to one embodiment, the patient display device is configured to encrypt the user input.

According to one embodiment, the display is configured to encrypt the user input for decryption by the implantable medical device.

According to one embodiment, the patient display device is configured to decrypt the control interface received from the patient external device, for displaying the control interface on the display.

According to one embodiment, at least one of the first and second application is configured to receive data from an auxiliary external device and present the received data to the user.

According to one embodiment, at least one of the first and second application is configured to receive data from an auxiliary external device comprising a scale for determining the weight of the user.

According to one embodiment, at least one of the first and second application is configured to receive data related to the weight of the user from an auxiliary external device comprising a scale.

According to one embodiment, the patient display device is configured to: wirelessly transmit the data related to the weight of the user to the patient external device, or wirelessly transmit an instruction derived from the data related to the weight of the user, or wirelessly transmit an instruction derived from a combination of the data related to the weight of the user and the implant control input received from the user.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A communication system for enabling communication between a patient display device and an implantable medical device, when implanted, is provided. The communication system comprises a patient display device,

a server or DDI, and a patient remote external device. The patient display device comprises a wireless communication unit configured for wirelessly receiving an implant control interface as a remote display portal from the patient remote external device, the wireless communication unit further being configured for wirelessly transmitting implant control user input to the patient remote external device, a display for displaying the received implant control interface as a remote display portal, and an input device for receiving implant control input from the user. The patient display device is configured to run a first application for wireless communication with the server, and to run a second application for wireless communication with the patient remote external device for transmission of the implant control input to the remote display portal of the patient remote external device for the communication with the implantable medical device. The patient remote external device comprises a wireless communication unit configured for wireless transmission of control commands based on the implant control input to the implantable medical device and configured for wireless communication with the patient display device.

According to one embodiment, the patient display device comprises a first log-in function and a second log-in function, and wherein the first log-in function gives the user access to the first application and wherein the first and second log-in function in combination gives the user access to the second application.

According to one embodiment, the second application is configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status,

a temperature, a time, or an error.

According to one embodiment, the patient display device is configured to encrypt the user input.

According to one embodiment, the display is configured to encrypt the user input for decryption by the implantable medical device.

According to one embodiment, the patient remote external device is configured to act as a router, transferring the encrypted user input from the patient display device to the implantable medical device without decryption.

According to one embodiment, the patient remote external device is configured to encrypt at least one of the control interface and the control commands.

According to one embodiment, the patient remote external device is configured to encrypt the control interface and wherein the patient display device is configured to decrypt the encrypted control interface.

A computer program product is provided, configured to run in a patient display device comprising a wireless communication unit, a display for displaying the received implant control interface as a remote display portal, and an input device for receiving implant control input from a user. The computer program product comprises:

a first application for communication with a server or DDI,

a second application for communication with an patient remote external device for transmission of the implant control input via the remote display portal of the patient remote external device for the communication with an implantable medical device, wherein the second application is configured to be accessed through the first application,

a first log-in function using at least one of a password, pincode, fingerprint, or face recognition, and

a second log-in function within the first application, using a private key from the user to authenticate for a defined time period a second hardware key of the patient remote external device. The first log-in function gives the user access to the first application and the first and second log-in function in combination gives the user access to the second application.

According to one embodiment, the second application is configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status,

a temperature, a time, or an error.

According to one embodiment of the communication system, patient display device or computer program product, the implantable medical device comprises at least one of:

an external heart compression device,

an apparatus assisting the pump function of a heart of the patient,

an apparatus assisting the pump function comprising a turbine bump placed within a patient's blood vessel for assisting the pump function of the heart,

an operable artificial heart valve,

an operable artificial heart valve for increasing the blood flow to the coronary arteries.

an implantable drug delivery device,

an implantable drug delivery device for injecting directly into a blood vessel and change the position of the injection site, all from within the patient's body,

an implantable drug delivery device for injecting potency enhancing drugs into an erectile tissue of the patient,

a hydraulic, mechanic, and/or electric constriction implant,

an operable volume filling device,

an operable gastric band,

an operable implant for stretching the stomach wall of the patient for creating satiety,

an implant configured to sense the frequency of the patient ingesting food,

an operable cosmetic implant,

an operable cosmetic implant for adjust the shape and/or size in the breast region of a patient,

an implant controlling medical device for the emptying of a urinary bladder,

an implant hindering urinary leakage,

an implant hindering anal incontinence,

an implant controlling the emptying of fecal matter,

an implant monitoring an aneurysm,

an implant for hindering the expansion of an aneurysm,

an implant lubricating a joint,

an implant for affecting the blood flow to an erectile tissue of the patient,

an implant for simulating the engorgement of an erectile tissue,

an implant with a reservoir for holding bodily fluids,

an implant storing and/or emptying a bodily reservoir or a surgically created

reservoir,

an implant communicating with a database outside the body,

an implant able to be programmed from outside the body,

an implant able to be programmed from outside the body with a wireless signal,

an implant treating impotence,

an implant controlling the flow of eggs in the uterine tube,

an implant controlling the flow of sperms in the uterine tube,

an implant controlling the flow of sperms in the vas deferens,

an implant for hindering the transportation of the sperm in the vas deferens,

an implant treating osteoarthritis,

an implant performing a test of parameters inside the body,

an implant controlling specific treatment parameters from inside the body,

an implant controlling bodily parameters from inside the body,

an implant controlling the blood pressure,

an implant controlling the blood pressure by affecting the dilatation of the renal artery,

an implant controlling a drug treatment parameter,

an implant controlling a parameter in the blood,

an implant for adjusting or replacing any bone part of a body of the patient,

an implant replacing an organ of the patient or part of an organ of the patient or the function thereof,

a vascular treatment device,

an implant adapted to move fluid inside the body of the patient,

an implant configured to sense a parameter related to the patient swallowing,

an implant configured to exercise a muscle with electrical or mechanical stimulation,

an implant configured for emptying an intestine portion on command,

an operable implant configured to be invaginated in the stomach of the patient to reduce the volume of the stomach substantially more than the volume of the device,

an implant configured for emptying the urinary bladder from within the patient's body by compressing the bladder,

an implant configured for draining fluid from within the patient's body,

an implant configured for the active lubrication of a joint with an added lubrication fluid,

an implant configured for removing clots and particles from the patient's blood stream,

an implant configured for elongating or straightening a bone in the patient, to reduce scoliosis,

a device to stimulate the brain for a several position to a focused point,

an artificial stomach replacing the function of the natural stomach,

an implant configured for adjusting the position of a female's urinary tract or bladder neck,

an implant configured for stimulating the ampulla vas deference and creating temporary constriction.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A communication system for enabling communication between a patient display device, a patient external device, a server and an implantable medical device, is provided. The communication system comprises a server,

a patient display device, a patient external device, and an implantable medical device.

The patient display device comprises a wireless communication unit for wirelessly communicating with at least one of the patient external device and the server, a display, and an input device for receiving input from the user. The patient external device comprises a wireless communication unit configured for wireless transmission of control commands to the implantable medical device and configured for wireless communication with at least one of the patient display device and the server. Further, the server comprises a wireless communication unit configured for wireless communication with at least one of the patient display device and the patient external device, wherein the implantable medical device comprises a wireless communication unit configured for wireless communication with the patient external device.

The implantable medical device further comprises an encryption unit and is configured to: encrypt data destined for the server, transmit the data to the server via the patient external device, wherein the patient external device acts as a router transferring the data without full decryption. In an example, the implantable medical device comprises an encryption unit and is configured to: encrypt data destined for the patient display device, transmit the data to the patient display device via the patient external device, wherein the patient external device acts as a router transferring the data without full decryption. In an example, the server comprises an encryption unit and is configured to: encrypt data destined for the implantable medical device, transmit the data to the implantable medical device via the patient external device, wherein the patient external device acts as a router transferring the data without full decryption, In an example, the server comprises an encryption unit and is configured to: encrypt data destined for the implantable medical device, transmit the data to the implantable medical device via the patient display device and the patient external device, wherein the patient display device and the patient external device acts as a router transferring the data without full decryption. In an example, the patient display device comprises an encryption unit and is configured to: encrypt data destined for the implantable medical device, transmit the data to the implantable medical device via the patient external device, wherein the patient external device acts as a router transferring the data without full decryption. In an example, the patient display device comprises an encryption unit and is configured to: encrypt data destined for the implantable medical device, transmit the data to the implantable medical device via the server and the patient external device, wherein the server and the patient external device acts as a router transferring the data without full decryption.

According to one embodiment, the patient display device is configured to wirelessly receive an implant control interface from the patient external device to be displayed on the display.

According to one embodiment, at least two of: the wireless communication unit of the server, the wireless communication unit of the patient display device, the wireless communication unit of the patient external device, and the wireless communication unit of the implantable medical device, are configured for wireless communication using a standard network protocol.

According to one embodiment, wherein at least two of: the wireless communication unit of the server, the wireless communication unit of the patient display device, the wireless communication unit of the patient external device, and the wireless communication unit of the implantable medical device, are configured for wireless communication using a proprietary network protocol.

According to one embodiment, the wireless communication unit of the patient external device is configured to use a first network protocol for communication with the implantable medical device and use a second network protocol for communication with the server, or use a first network protocol for communication with the implantable medical device and use a second network protocol for communication with the patient display device.

According to one embodiment, the wireless communication unit of the patient external device is configured to use a first frequency band for communication with the implantable medical device and use a second frequency band for communication with the server, or use a first frequency band for communication with the implantable medical device and use a second frequency band for communication with the patient display device.

According to one embodiment, the wireless communication unit of the patient display device is configured to use a first network protocol for communication with the patient external device and use a second network protocol for communication with the server.

According to one embodiment, the wireless communication unit of the patient display device is configured to use a first frequency band for communication with the patient external device and use a second frequency band for communication with the server.

According to one embodiment, the wireless communication unit of the server is configured to use a first network protocol for communication with the patient external device and use a second network protocol for communication with the patient display device.

According to one embodiment, the wireless communication unit of the server is configured to use a first frequency band for communication with the patient external device and use a second frequency band for communication with the patient display device.

According to one embodiment, the wireless communication unit of at least one of the server, the patient display device, the patient external device, and the implantable medical device comprises a Bluetooth transceiver.

According to one embodiment, the wireless communication unit of at least one of the server, the patient display device, the patient external device, and the implantable medical device comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the wireless communication unit of the patient external device comprises a first wireless transceiver for wireless communication with the implantable medical device, and a second wireless transceiver for wireless communication with the server, and wherein the second wireless transceiver has a longer effective range than the first wireless transceiver.

According to one embodiment, the wireless communication unit of the patient external device comprises a first wireless transceiver for wireless communication with the implantable medical device, and a second wireless transceiver for wireless communication with the patient display device, and wherein the second wireless transceiver has a longer effective range than the first wireless transceiver.

According to one embodiment, the wireless communication unit of the patient display device comprises a first wireless transceiver for wireless communication with the patient external device, and a second wireless transceiver for wireless communication with the server, and wherein the second wireless transceiver has a longer effective range than the first wireless transceiver.

According to one embodiment, the second wireless transceiver has an effective range being one of: 2 times, 4 times, 8 times 20 times, 50 times or 100 times longer than the first wireless transceiver.

According to one embodiment, the second wireless transceiver is configured to be disabled to enable wireless communication using the first wireless transceiver.

According to one embodiment, at least one of:

the patient display device is configured to authenticate the patient external device if a distance between the patient display device and the patient external device is less than a predetermined threshold value,

the patient display device is configured to be authenticated by the patient external device if a distance between the patient display device and the patient external device is less than a predetermined threshold value,

the patient display device is configured to authenticate the implantable medical device if a distance between the patient display device and the implantable medical device is less than a predetermined threshold value,

the patient display device is configured to be authenticated by the implantable medical device if a distance between the patient display device and the implantable medical device is less than a predetermined threshold value,

the patient external device is configured to authenticate the patient display device if a distance between the patient external device and the patient display device is less than a predetermined threshold value,

the patient external device is configured to be authenticated by the patient display device if a distance between the patient external device and the patient display device is less than a predetermined threshold value,

the patient external device is configured to authenticate the implantable medical device if a distance between the patient external device and the implantable medical device is less than a predetermined threshold value, and

the patient external device is configured to be authenticated by the implantable medical device if a distance between the patient external device and the implantable medical device is less than a predetermined threshold value.

According to one embodiment, the patient display device is configured to allow the transfer of data between the patient display device and the patient external device on the basis of the authentication.

According to one embodiment, the patient external device is configured to allow the transfer of data between the patient display device and the patient external device on the basis of the authentication.

According to one embodiment, the patient external device is configured to allow the transfer of data between the patient external device and the implantable medical device on the basis of the authentication.

According to one embodiment, the patient display device is a wearable patient external device or a handset.

According to one embodiment, the data encrypted by the implantable medical device is related to at least one of: a battery status, a temperature, a time, or an error.

A server for use in the communication system according to any one of the above embodiments is provided.

A patient display device for use in the communication system according to any one of the above embodiments is provided.

A patient external device for use in the communication system according to any one of the above embodiments is provided.

An implantable medical device for use in the communication system according to any one of the above embodiments is provided.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system configured for changing pre-programmed treatment settings of an implantable medical device, when implanted in a patient, from a distant remote location in relation to the patient, is provided. The system comprises at least one health care provider, HCP, EID external device, and a HCP private key device. HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication. The HCP EID external device comprises at least one of: a reading slot or comparable for the HCP private key device, a RFID communication, and other close distance wireless activation communication or electrical direct contact.

The HCP EID external device further comprises at least one wireless transceiver configured for communication with a data infrastructure server, DDI, through a first network protocol. Further, the system comprises a data infrastructure server, DDI, adapted to receive command from said HCP EID external device and to relay the received command without modifying said command to a patient EID external device, wherein the DDI comprises one wireless transceiver configured for communication with said patient external device, and a patient EID external device adapted to receive the command relayed by the DDI, further adapted to send this command to the implanted medical device, further adapted to receive a command from the HCP EID external device via the DDI to change said pre-programmed treatment settings of the implanted medical device, and further adapted to be activated and authenticated and allowed to perform said command by the patient providing a patient private key device adapted to be provided to the patient EID external device by the patient via at least one of: a reading slot or comparable for the patient private key device, a RFID communication or other close distance wireless activation communication or electrical direct contact. The patient EID external device comprises at least one of a reading slot or comparable for the HCP private key device, a RFID communication, and other close distance wireless activation communication or electrical direct contact. The patient EID external device further comprises at least one wireless transceiver configured for communication with the implanted medical device through a second network protocol. Further, the implanted medical device is configured to treat the patient or perform a bodily function.

According to one embodiment, at least one of the patient private key device or HCP private key device comprises a hardware key.

According to one embodiment, the private key device is at least one of, a smartcard, a key-ring device, a watch an arm or wrist band a neckless or any shaped device.

According to one embodiment of the system, at least two of: the HCP EID external device, the patient EID external device, the HCP private key device, the patient private key device, and the DDI are configured for wireless communication using a standard network protocol.

According to one embodiment, at least two of: the HCP EID external device, the patient EID external device, the HCP private key device, the patient private key device, and the DDI are configured for wireless communication using a proprietary network protocol.

According to one embodiment, the patient EID external device is configured to use a first network protocol for communication with the implantable medical device and use a second network protocol for communication with the DDI.

According to one embodiment, the patient EID external device is configured to use a first frequency band for communication with the implantable medical device and use a second frequency band for communication with the DDI.

According to one embodiment, the DDI is configured to use a first frequency band for communication with the patient EID external device and a second frequency band for communication with the patient private key device.

According to one embodiment, at least one of the HCP EID external device, the patient EID external device, the HCP private key device, the patient private key device and the DDI comprises a Bluetooth transceiver.

According to one embodiment, at least one of the HCP EID external device, the patient EID external device, the HCP private key device, the patient private key device and the DDI comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the DDI, and wherein the second wireless transceiver has longer effective range than the first wireless transceiver.

According to one embodiment, the patient private key device comprises a first wireless transceiver for wireless communication with the HCP EID external device, and a second wireless transceiver for wireless communication with the DDI, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is configured to allow transfer of data between the EID external device and the implantable medical device on the basis of an authentication of the patient EID external device.

According to one embodiment, the patient EID external device is a wearable patient external device or a handset.

According to one embodiment, the data encrypted by the implantable medical device is related to at least one of: a battery status, a temperature, a time, or an error.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, configured for changing pre-programmed treatment settings of an implantable medical device, when implanted in a patient, by a health care provider, HCP, in the physical presence of the patient. The system comprises at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing an HCP private key device comprising a HCP private key. The HCP private key device comprises at least one of: a smart card, a keyring device, a watch, a arm or wrist band, a necklace, and any shaped device. The HCP EID external device is adapted to be involved in at least one of: receiving information from the implant, receiving information from a patient remote external device, actuating the implanted medical device, changing pre-programmed settings, and updating software of the implantable medical device, when implanted. The HCP EID external device is further adapted to be activated, authenticated, and allowed to perform said command also by the patient. The system further comprises a patient private key device comprising a patient private key, wherein the patient private key device comprising at least one of: a smart card, a keyring device, a watch, a arm or wrist band, a necklace, and any shaped device. The HCP private key and the patient private key are required for performing said actions by the HCP EID external device to at least one of: receive information from the implant, to receive information from a patient remote external device, to actuate the implanted medical device, to change pre-programmed settings, and to update software of the implantable medical device, when the implantable medical device is implanted.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of: a reading slot or comparable for the HCP private key device, a RFID communication, and a close distance wireless activation communication unit, or electrical direct contact.

According to one embodiment, the HCP EID external device comprises at least one of reading slot or comparable for the HCP private key device,

a RFID communication and a close distance wireless activation communication unit, or electrical direct contact.

According to one embodiment, the HCP EID external device is adapted to receive a command from a HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, at least two of: the HCP EID external device, the patient EID external device, the HCP private key device, and the patient private key device, are configured for wireless communication using a standard network protocol.

According to one embodiment, at least two of: the HCP EID external device, the patient EID external device, the HCP private key device, and the patient private key device, are configured for wireless communication using a proprietary network protocol.

According to one embodiment, the patient EID external device is configured to use a first network protocol for communication with the implantable medical device and use a second network protocol for communication with the patient private key device.

According to one embodiment, the patient EID external device is configured to use a first frequency band for communication with the implantable medical device and use a second frequency band for communication with the patient private key device.

According to one embodiment, at least one of the HCP EID external device, the patient EID external device, the HCP private key device, and the patient private key device comprises a Bluetooth transceiver.

According to one embodiment, at least one of the HCP EID external device, the patient EID external device, the HCP private key device, and the patient private key device comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the patient private key device, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is configured to allow transfer of data between the EID external device and the implantable medical device on the basis of an authentication of the patient EID external device.

According to one embodiment, the patient EID external device is a wearable patient external device or a handset.

According to one embodiment, the data encrypted by the implantable medical device is related to at least one of: a battery status, a temperature, a time, or an error.

A system is provided, configured to change pre-programmed and pre-selected treatment actions of an implantable medical device, when implanted in a patient, by command from the patient.

The system comprises an implantable medical device, a patient remote external device, a wireless transceiver configured for communication with the implantable medical device, when the medical device is implanted, through a second network protocol, and a remote display portal. The remote display portal is configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualised on the patient display device.

According to one embodiment, the wireless transceiver, the remote display portal, and the remote display portal are comprised in the patient remote external device.

According to one embodiment, the system further comprises the patient display device, which may comprise a supporting application, a display which hosts the Remote Display Portal, and a patient display device private key.

According to one embodiment, the remote display portal is capable of generating a command to be signed by the patient display device private key.

According to one embodiment, the patient remote external device is adapted to accept input from the patient via said patient display device through its remote display portal.

According to one embodiment, the patient remote external device comprises a graphical user interface arranged on a touch-responsive display exposing buttons to express actuation functions of the implanted medical device.

According to one embodiment, the system is configured to allow the patient to actuate the implant at home through the patient remote external device by means of an authorization granted by a patient private key.

According to one embodiment, the patient private key comprises at least one of: a smart card, a keyring device, a watch, a arm or wrist band, a necklace, and any shaped device.

According to one embodiment, the system is configured to allow the patient to actuate the implantable medical device, when implanted, at home through the patient remote external device, using an authorization granted by the patient private key.

According to one embodiment, system further comprises a patient EID external device comprising at least one of: a reading slot or comparable for the patient private key device, a RFID communication, and a close distance wireless activation communication, or electrical direct contact.

According to one embodiment, the patient EID external device is adapted to be synchronised with the patient remote external device.

According to one embodiment, the patient EID external device further comprises at least one of: a wireless transceiver configured for communication with the patient, a remote external device, and a wired connector for communication with the patient remote external device.

According to one embodiment, the patient EID external device is adapted to generate an authorization to be signed by the patient private key to be installed into at least one of: the patient remote external device through the patient EID external device, and the implantable medical device.

According to one embodiment, the system comprises a patient display device comprising a supporting application capable of displaying the remote display portal with content delivered from the patient remote external device.

According to one embodiment, the remote display portal and patient remote external device are adapted to expose buttons to express the will to actuate the functions of the implanted medical device by the patient through the patient remote external device.

According to one embodiment, the patient display device comprises at least one of: a display which hosts the remote display portal, and a patient display device private key.

According to one embodiment, the remote display portal is capable of generating a command to be signed by the patient private key.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, configured for providing information from an implantable medical device, when implanted in a patient, from a distant remote location in relation to the patient. The system comprises at least one patient EID external device adapted to receive information from the implant, adapted to send such information further on to a server or dedicated data infrastructure, DDI, further adapted to be activated and authenticated and allowed to receive said information by the implanted medical device by the patient providing a private key. Further, the system comprises a patient private key device comprising the private key adapted to be provided to the patient EID external device via at least one of: a reading slot or comparable for the patient private key device, a RFID communication or other close distance wireless activation communication or direct electrical connection. The patient EID external device comprises at least one of: a reading slot or comparable for the patient private key device, an RFID communication, and other close distance wireless activation communication or direct electrical contact. Further, the patient EID external device comprises at least one wireless transceiver configured for communication with the DDI, through a first network protocol.

According to one embodiment, the at least one patient EID external device is adapted to receive information from the implant, through a second network protocol.

According to one embodiment, the system comprises the DDI, wherein the DD1 is adapted to receive information from said patient EID external device, and wherein the DDI comprises a wireless transceiver configured for communication with said patient EID external device.

According to one embodiment, the patient EID external device is adapted to receive a command relayed by the DDI, to further send the command to the implanted medical device to change said pre-programmed treatment settings of the implanted medical device, and further adapted to be activated and authenticated and allowed to perform said command by the patient providing the patient private key.

According to one embodiment, the patient private key device is adapted to provide the patient private key to the patient EID external device by the patient via at least one of; a reading slot or comparable for the patient private key device, an RFID communication or other close distance wireless activation communication, or electrical direct contact.

According to one embodiment, the patient EID external device comprises at least one of: a reading slot or comparable for the HCP private key device, a RFID communication, and other close distance wireless activation communication, or direct electrical contact.

According to one embodiment, the patient EID external device further comprising at least one wireless transceiver configured for communication with the implanted medical device through a second network protocol.

According to one embodiment, the system comprises the implantable medical device, which may be adapted to, when implanted, treat the patient or perform a bodily function.

According to one embodiment, the patient private key comprises at least one of: a smart card, a keyring device, a watch, an arm band or wrist band, a necklace, and any shaped device.

According to one embodiment, at least two of: the patient EID external device, the IDD, and the patient private key device, are configured for wireless communication using a standard network protocol.

According to one embodiment, at least two of: the patient EID external device, the IDD, and the patient private key device, are configured for wireless communication using a proprietary network protocol.

According to one embodiment, the patient EID external device is configured to use a first network protocol for communication with the implantable medical device and use a second network protocol for communication with the patient private key device.

According to one embodiment, the patient EID external device is configured to use a first frequency band for communication with the implantable medical device and use a second frequency band for communication with the patient private key device.

According to one embodiment, at least one of the patient EID external device, the patient private key device and the IDD comprises a Bluetooth transceiver.

According to one embodiment,

at least one of the patient EID external device, the patient private key device and the IDD comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the patient private key device, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is a wearable patient external device or a handset.

According to one embodiment, the data encrypted by the implantable medical device is related to at least one of: a battery status, a temperature, a time, or an error.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, comprising, an implantable medical device adapted to, when implanted in a patient, to communicate with an external device, the external device comprising at least one of a patient remote external device or a patient EID external device. The system further comprises the patient EID external device adapted to communicate with and send commands to the implantable medical device when implanted, to change pre-programmed settings, and a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact. Further the system comprises a data infrastructure server, DDI, adapted to send commands to the patient EID external device for further transport to the implanted medical device, to inactivate the authority and authenticating function of the patient private key.

According to one embodiment, the at least one patient remote external device comprises a patient remote external device private key, wherein the DDI via the patient EID external device is able to inactivate the authority and authenticating function of the patient remote external device, thereby inactivating the patient remote external device.

According to one embodiment, the patient EID external device comprises at least one wireless transceiver configured for communication with the DD1 via a first network protocol.

According to one embodiment, the system comprises the DDI, wherein the DDI is adapted to receive command from a HCP EID external device, and to send the received command to the patient EID external device, wherein the DDI comprises a wireless transceiver configured for communication with said patient external device.

According to one embodiment, the patient EID external device is adapted to receive the command from the DDI, wherein the command originates from a health care provider, HCP, and wherein the patient EID is adapted to inactivate the patient private key and to send the command to the implanted medical device.

According to one embodiment, the patient EID external device is adapted to receive the command from the DDI, wherein the command originates from a health care provider, HCP, wherein the patient EID external device is adapted to receive the command from the HCP via the DDI to inactivate the patient remote external device comprising a patient remote external device private key, and wherein the patient EID external device is further adapted to send this command to the implanted medical device.

According to one embodiment, the patient EID external device further comprises at least one wireless transceiver configured for communication with the implanted medical device through a second network protocol.

According to one embodiment, at least one of the patient private key and a patient remote external device private key comprises a hardware key.

According to one embodiment, the private key device is at least one of, a smartcard, a key-ring device, a watch an arm or wrist band a neckless or any shaped device.

According to one embodiment, at least two of: the patient remote external device, the patient EID external device, the patient private key device, and the DDI, are configured for wireless communication using a standard network protocol.

According to one embodiment, wherein at least two of: the patient remote external device, the patient EID external device, the patient private key device, and the DDI, are configured for wireless communication using a proprietary network protocol.

According to one embodiment, the patient EID external device is configured to use a first network protocol for communication with the implantable medical device and use a second network protocol for communication with the patient private key device.

According to one embodiment, the patient EID external device is configured to use a first frequency band for communication with the implantable medical device and use a second frequency band for communication with the patient private key device.

According to one embodiment, at least one of the patient remote external device, the patient EID external device, the patient private key device, and the DDI, comprise a Bluetooth transceiver.

According to one embodiment, at least one of the patient remote external device, the patient EID external device, the patient private key device, and the DDI, comprise an UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the patient private key device, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is a wearable patient external device or a handset.

According to one embodiment, the data encrypted by the implantable medical device is related to at least one of: a battery status, a temperature, a time, or an error.

According to one embodiment, the system comprises a master private key device configured to allow issuance of new private key device, wherein the HCP or HCP admin have such master private key device adapted to be able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, configured for changing pre-programmed treatment settings in steps of an implantable medical device, when implanted in a patient, by a health care provider, HCP, either in the physical presence of the patient or remotely with the patient on distance. The system comprises at least one HCP EID external device adapted to receive a command directly or indirectly from the HCP to change said pre-programmed treatment settings in steps of the implantable medical device, when implanted. The HCP EID external device is further adapted to be activated, authenticated, and allowed to perform said command by the HCP providing a HCP private key device comprising a HCP private key. The HCP private key comprises at least one of: a smart card, a keyring device, a watch, an arm or wrist band, a necklace, and any shaped device. The system further comprises a patient private key device comprising a patient private key, comprising at least one of: a smart card, a keyring device, a watch, an arm or wrist band, a necklace, and any shaped device. Both the HCP and patient private key is required for performing said action by the HCP EID external device to change the pre-programmed settings in the implant and to update software of the implantable medical device, when the implantable medical device is implanted. The patient private key is adapted to activate, be authenticated, and allowed to perform said command provided by the HCP, either via the HCP EID external device or when the action is performed remotely via a patient EID external device.

According to one embodiment, the system comprises a master private key device that allow issuance of new private key device wherein the HCP or HCP admin have such master private key device adapted to be able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system further comprises a measurement device or sensor adapted to deliver a measurement to at least one of the DDI, patent EID external device and a patient display device.

According to one embodiment, the system further comprises a food sensor adapted to measure at least if the patient swallows solid food or is drinking fluid, wherein said food sensor is configured to be connected to the control unit of a medical device to cause an action to stretch the stomach after a determined amount of food intake.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of: a reading slot or comparable for the HCP private key device, a RFID communication, and a close distance wireless activation communication unit, or electrical direct contact.

According to one embodiment, the HCP EID external device comprises at least one of: reading slot or comparable for the HCP private key device, a RFID communication, and a close distance wireless activation communication unit, or electrical direct contact.

According to one embodiment, the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, the HCP EID external device and the HCP private key device are configured for wireless communication using a standard network protocol.

According to one embodiment, the HCP EID external device and the HCP private key device are configured for wireless communication using a proprietary network protocol.

According to one embodiment, the HCP EID external device is configured to use a first network protocol for communication with the implantable medical device and use a second network protocol for communication with the HCP private key device.

According to one embodiment, the HPC EID external device is configured to use a first frequency band for communication with the implantable medical device and use a second frequency band for communication with the HCP private key device.

According to one embodiment, at least one of the HCP EID external device and the HCP private key device comprises a Bluetooth transceiver.

According to one embodiment, at least one of the HCP EID external device and the HCP private key device comprises a UWB transceiver.

A system is provided, configured for changing pre-programmed treatment settings in steps of an implantable medical device, when implanted in a patient, by a health care provider, HCP, with the patient on remote on distance. The system comprises at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP. The action by the HCP EID external device to change pre-programmed settings in the implant and to update software of the implantable medical device, when the implantable medical device is implanted, is adapted to be authenticated by a HCP private key device and a patient private key device.

According to one embodiment, the HCP private key device comprising a HCP private key, comprising at least one of: a smart card, a keyring device, a watch, an arm or wrist band, a necklace, and any shaped device.

According to one embodiment, the patient private key device comprises a patient private key, comprising at least one of: a smart card, a keyring device, a watch, an arm or wrist band, a necklace, and any shaped device.

According to one embodiment, the patient private key is adapted to activate, be authenticated, and allowed to perform said command provided by the HCP, either via the HCP EID external device or when the action is performed remotely via a patient EID external device.

According to one embodiment, the system further comprises a dedicated data infrastructure, DDI, the patient EID external device, and the HCP EID external device, wherein the communication between the patient EID external device and the HCP EID external device is performed via the DDI.

According to one embodiment, the system comprises a master private key device that allows issuance of new private key device wherein the HCP or HCP admin have such master private key device adapted to be able to replace and pair a new patient private key device or HCP private key device into the system.

According to one embodiment, the patient remote external device and the patient EID external device are an integrated unit.

According to one embodiment, the HCP dedicated device and the HCP EID external device are an integrated unit.

According to one embodiment, the system further comprises a measurement device or sensor adapted to deliver a measurement to at least one of the DDI, patent EID external device and patient display device.

According to one embodiment, the system comprises a food sensor, adapted to measure at least if the patient swallow solid food or is drinking fluid, wherein said food sensor is connected to the control unit of a medical device to cause an action to stretch the stomach after a determined amount of food intake.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of: a reading slot or comparable for the HCP private key device, a RFID communication, and a close distance wireless activation communication unit, or electrical direct contact.

According to one embodiment, the HCP EID external device comprises at least one of: reading slot or comparable for the HCP private key device, a RFID communication, and a close distance wireless activation communication unit, or electrical direct contact.

According to one embodiment, the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, the HCP EID external device and the HCP private key device are configured for wireless communication using a standard network protocol.

According to one embodiment, the HCP EID external device and the HCP private key device are configured for wireless communication using a proprietary network protocol.

According to one embodiment, the HCP EID external device is configured to use a first network protocol for communication with the implantable medical device and use a second network protocol for communication with the HCP private key device.

According to one embodiment, the HPC EID external device is configured to use a first frequency band for communication with the implantable medical device and use a second frequency band for communication with the HCP private key device.

According to one embodiment, at least one of the HCP EID external device and the HCP private key device comprises a Bluetooth transceiver.

According to one embodiment, at least one of the HCP EID external device and the HCP private key device comprises a UWB transceiver.

A system is provided, which is configured for changing pre-programmed treatment settings of an implantable medical device, when implanted in a patient, from a distant remote location in relation to the patient. The system comprises at least one health care provider, HCP, external device adapted to receive a command from the HCP to change said pre-programmed treatment settings of an implanted medical device. The HCP external device is further adapted to be activated and authenticated and allowed to perform said command by the HCP providing a HCP private key device adapted to be provided to an HCP EID external device via at least one of; a reading slot or comparable for the HCP private key device, a RFID communication or other close distance wireless activation communication. The HCP EID external device comprises at least one of: a reading slot or comparable for the HCP private key device, a RFID communication, and other close distance wireless activation communication or electrical direct contact. The HCP EID external device further comprises at least one wireless transceiver configured for communication with a patient EID external device, through a first network protocol. The system comprises the patient EID external device, the patient EID external device being adapted to receive command from said HCP external device, and to relay the received command without modifying said command to the implanted medical device. The patient EID external device comprises one wireless transceiver configured for communication with said patient external device, wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.

According to one embodiment, at least one of the patient private key device or HCP private key device comprises a hardware key.

According to one embodiment, the private key device is at least one of, a smartcard, a key-ring device, a watch an arm or wrist band a neckless or any shaped device.

According to one embodiment, the system comprises a master private key device that allow issuance of new private key device wherein the HCP or HCP admin have such master private key device adapted to be able to replace and pair a new patient private key device or HCP private key device into the system, through the HCP EID external device.

According to one embodiment, the patient remote external device and the patient EID external device is an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallow solid food or is drinking fluid, wherein said food sensor is connected to the control unit of a medical device to cause an action to stretch the stomach after a determined amount of food intake.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of; a reading slot or comparable for the HCP private key device, a RFID communication, and a close distance wireless activation communication unit, or electrical direct contact.

According to one embodiment, the HCP EID external device comprises at least one of: reading slot or comparable for the HCP private key device, a RFID communication, and a close distance wireless activation communication unit, or electrical direct contact.

According to one embodiment, the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, the HCP EID external device and the HCP private key device are configured for wireless communication using a standard network protocol.

According to one embodiment, the HCP EID external device and the HCP private key device are configured for wireless communication using a proprietary network protocol.

According to one embodiment, the HCP EID external device is configured to use a first network protocol for communication with the implantable medical device and use a second network protocol for communication with the HCP private key device.

According to one embodiment, the HPC EID external device is configured to use a first frequency band for communication with the implantable medical device and use a second frequency band for communication with the HCP private key device.

According to one embodiment, at least one of the HCP EID external device and the HCP private key device comprises a Bluetooth transceiver.

According to one embodiment, at least one of the HCP EID external device and the HCP private key device comprises a UWB transceiver.

A medical device for actively stretching a stomach wall of a patient for creating a sensation of satiety is further provided. The medical device is implantable and comprises a second member configured to be fixated to the stomach wall of the patient and comprising a closed curve and a first member configured to be fixated to the stomach wall of the patient within the footprint of the second member. The first member is operably connected to the second member such that the first member can be displaced relative to the second member, and within the footprint of the second member—for stretching a portion of the stomach wall of the patient between the second and first member.

According to one embodiment, the second member is configured to encircle a portion of the stomach wall of the patient.

According to one embodiment, the second member comprises a simple closed curve, which may mean that the second member is ring shaped.

According to one embodiment, the first member is configured to encircle a portion of the stomach wall of the patient.

According to one embodiment, the first member comprises a simple closed curve, which may mean that the first member is ring shaped.

The first and/or second members may be configured to be invaginated by the stomach wall.

According to one embodiment, the first member is configured to be mechanically operable and the medical device may further comprising a mechanical operation device for operating the first member. The mechanical operation device may comprise at least one flexible element, directly or indirectly fixated to the first member. The flexible element may be configured to operate the first member for moving the first member in a first direction towards the second member.

The mechanical operation device may further comprise at least one second flexible element, directly or indirectly fixated to the first member. The second flexible element is configured to operate the first member for moving the first member in a second direction towards the second member, the second direction being different from the first direction.

According to one embodiment, the mechanical operation device comprises at least one third flexible element, directly or indirectly fixated to the first member. The third flexible element being configured to operate the first member for moving the first member in a third direction towards the second member, the third direction being different from the first and second directions.

The second member may comprise at least one recess or channel, and at least one of the flexible elements are configured to travel at least partially in the recess or channel.

According to one embodiment, the medical device further comprises at least one sheath, and the at least one flexible element is configured to travel through the sheath.

According to one embodiment, the sheath comprises elevated and lowered portions, such that the sheath can remain flexible even as fibrotic tissue overgrows the sheath, e.g., at least one portion of the sheath is shaped like a bellows.

According to one embodiment, the sheath is connected to the first and second members, such that at least one flexible element can travel from the first to the second member through the sheath.

The mechanical operation device may comprise at least one pulley, and the at least one flexible element may be configured to engage the pulley such that transmission is created.

According to one embodiment, the pulley is connected to the first member and a first flexible element is configured to travel from a first point on the second member to the pulley such that the pulley is moved closer to the first point by the first flexible element being pulled in a direction towards the first point, and the transmission created by the pulley causes the pulley to move towards the first point at a lower speed than the first flexible element moves towards the first point.

According to one embodiment, the medical device further comprises a second pulley connected to the first member, and a second flexible element is configured to travel from a second point on the second member to the second pulley such that the second pulley is moved closer to the second point by the second flexible element being pulled in a direction towards the second point, and the transmission created by the second pulley causes the second pulley to move towards the second point at a lower speed than the second flexible element moves towards the second point.

According to one embodiment, the mechanical operation device comprises at least one movable arm having a first portion connected to the first member, and a second portion connected to the second member. The at least one movable arm may comprise at least one operable joint and the at least one movable arm may be configured to move the first member relative to the second member in at least a first direction and a second direction, where the second direction is opposite the first direction. The at least one movable arm may further be configured to move the first member relative to the second member in at least a third direction different from the first and second direction.

According to one embodiment, a first portion of a flexible element is directly or indirectly fixated to a first portion of the first member, and a second portion of the flexible element is directly or indirectly fixated to a second portion of the first member.

According to one embodiment, the flexible element is connected to a propulsion device for propelling the flexible element, and propulsion of the flexible element in a first direction causes the first member to move towards a first point on the second member, and propulsion of the flexible element in a second direction causes the first member to move towards a second point on the second member.

According to one embodiment, the first member is hydraulically operable, and may further comprising a hydraulic operation device for operating the first member. The hydraulic operation device may comprise at least one hydraulic actuator, directly or indirectly fixated to the first and second members, and the hydraulic actuator is configured to operate the first member for moving the first member in a first direction towards the second member.

According to one embodiment, the hydraulic operation device comprises at least one second hydraulic actuator, directly or indirectly fixated to the first and second members. The second hydraulic actuator may be configured to operate the first member for moving the first member in a second direction towards the second member, the second direction being different from the first direction. The medical device may further comprise at least one hydraulic conduit for conducting a hydraulic fluid to at least one of the hydraulic actuators.

At least one of the first and second members may comprise at least one recess or channel, and the hydraulic conduit may travel at least partially in the recess or channel. At least one of the first and second members may comprise at least one channel for conducting a hydraulic fluid.

According to one embodiment, the medical device further comprises at least one sheath, and at least one hydraulic actuator may be configured to travel through the sheath. The sheath may comprise elevated and lowered portions, such that the sheath can remain flexible even as fibrotic tissue overgrows the sheath.

According to one embodiment, the first member is substantially rigid.

According to one embodiment, a major portion of the second member has a modulus of elasticity in the range 50 GPa-400 GPa, which may be an effect of the second member being made from a material having a modulus of elasticity in the range 50 GPa-400 GPa.

According to one embodiment, the medical device may be configured to be completely invaginated by the stomach wall, apart from elements for transferring force or communication to or from the medical device.

According to one embodiment, the first or second member may be operable for adjusting at least one of the length of the closed curve and the cross-sectional area of the closed curve, for calibrating the stretching of the stomach wall. The first or second member may be mechanically operable for adjusting at least one of the length of the closed curve and the cross-sectional area of the closed curve, for calibrating the stretching of the stomach wall. The first or second member may comprise a rotatable threaded member for adjusting the length of the closed curve, for calibrating the stretching of the stomach wall.

According to one embodiment, the first or second member is hydraulically operable for adjusting at least one of the length of the closed curve and the cross-sectional area of the closed curve, for calibrating the stretching of the stomach wall. The first or second member may comprise at least one inflatable member for adjusting at least one of the length of the closed curve and the cross-sectional area of the closed curve, for calibrating the stretching of the stomach wall.

According to one embodiment, the first member comprises at least one portion comprising elevated and lowered areas for enabling adjustment of at least one of the length of the closed curve and the cross-sectional area of the closed curve, e.g., the first member may comprise at least one bellows-shaped portion for enabling adjustment of at least one of the length of the closed curve and the cross-sectional area of the closed curve.

According to one embodiment, a portion of the mechanical or hydraulic operation device may be placed in a remote unit configured to be placed at a remote location in the body of the patient, and the medical device may further comprise a force transferring element configured to transfer force hydraulically or mechanically from the remote unit to the first member, for operating the first member.

According to one embodiment, the portion of the operation device placed in the remote unit comprises at least one electrical motor and a gear system configured to reduce the speed and increase the force of the movement generated by the electrical motor.

According to one embodiment, the portion of the operation device placed in the remote unit may comprise at least one pulley for propelling the flexible element.

According to one embodiment, the portion of the operation device placed in the remote unit comprises at least one hydraulic pump. The remote unit may further comprise a hydraulic reservoir for holding a hydraulic fluid.

According to one embodiment, the remote unit comprises an injection port for at least one of injecting and removing hydraulic fluid from the medical device.

According to one embodiment, the remote unit further comprises an energy storage unit for directly or indirectly energizing the medical device.

According to one embodiment, the remote unit further comprises a controller for controlling the operation device.

According to one embodiment, the controller comprises a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

A medical device for actively stretching a stomach wall of a patient for creating a sensation of satiety is further provided. The medical device is implantable and comprises a second member configured to be fixated to the stomach wall of the patient, and an operable first member connected to the second member and configured to be fixated to the stomach wall at least partially within the footprint of the second member. The first member is configured to be operated to deform such that a first portion of the first member is moved away from a first portion of the second member, for stretching a portion of the stomach wall between the first portion of the first member and the first portion of the second member.

According to one embodiment, the first member comprises at least one curvature, e.g., at least a portion of the first member forms a circular arc. According to one embodiment, a portion of the first member is configured to deform, and the portion of the first member configured to deform may comprise at least one of the curvature and the circular arc.

According to one embodiment, the second member comprises at least one curvature, e.g., at least a portion of the second member forms a circular arc. According to one embodiment, the second member is ring-shaped.

According to one embodiment, the circular are of the first member has a first radius, and the circular are of the second member has a second radius, and the second radius is larger than the first radius.

According to one embodiment, at least one portion of the second member is fixedly fixated to the first member, and the second member may comprise a connecting portion for connecting the second member to the first member.

According to one embodiment, at least two portions of the second member may be fixedly fixated to the first member.

According to one embodiment, the first member may be fixated to the second member such that the connection with the second member provides counterforce against the force needed for the first member to deform.

According to one embodiment, the footprint of the first member is configured to enclose a portion of the stomach wall.

According to one embodiment, the footprint of the second member is configured to enclose a portion of the stomach wall.

According to one embodiment, the footprint of the first member is smaller than the footprint of the second member.

According to one embodiment, the second member is configured to encircle a portion of the stomach wall of the patient, and the second member may comprise a simple closed curve.

According to one embodiment, the first member is configured to encircle a portion of the stomach wall of the patient, and the first member may comprise a simple closed curve.

The for and/or second member may in any of the embodiments be configured to be invaginated by the stomach wall.

According to one embodiment, the first member is further configured to be operated to deform such that a second portion of the first member is moved away from a second portion of the second member, for stretching a portion of the stomach wall between the second portion of the first member and the second portion of the second member.

According to one embodiment, the first member is further configured to be operated to deform such that a third portion of the first member is moved away from a third portion of the second member, for stretching a portion of the stomach wall between the third portion of the first member and the third portion of the second member.

According to one embodiment, the first, second and third portions are positioned substantially equidistantly from each other.

According to one embodiment, the first member is mechanically operable and may further comprise a mechanical operation device for operating the first member.

According to one embodiment, the mechanical operation device comprises at least one flexible element, directly or indirectly fixated to the first member. The flexible element may be configured to operate the first member such that the first member deforms.

According to one embodiment, a first portion of the flexible element is directly or indirectly fixated to a first portion of the flexible element, and a second portion of the flexible element is directly or indirectly fixated to a second portion of the flexible element. The flexible element is configured to operate the first member such that the first and second portion deforms.

According to one embodiment, the flexible element is connected to a propulsion device for propelling the flexible element, and propulsion of the flexible element in a first direction causes the first member to deform such that the first portion of the first member moves away from a first portion of the second member, and propulsion of the flexible element in a second direction causes the first member to deform such that the second portion of the first member moves away from a second portion of the second member.

According to one embodiment, the mechanical operation device comprises at least one second flexible element, directly or indirectly fixated to a second portion of the first member. The second flexible element is configured to operate the first member to deform such that a second portion of the first member is moved away from a second portion of the second member, for stretching a portion of the stomach wall between the second portion of the first member and the second portion of the second member.

According to one embodiment, the mechanical operation device comprises at least one third flexible element, directly or indirectly fixated to a third portion of the first member. The third flexible element may be configured to operate the first member to deform such that a third portion of the first member is moved away from a third portion of the second member, for stretching a portion of the stomach wall between the third portion of the first member and the third portion of the second member.

According to one embodiment, the first portion is a flexible portion, and the first member is configured to deform by deformation of the at least one flexible portion.

According to one embodiment, the first flexible portion comprises at least one elastic portion and the elastic portion may be elastic by means of the elastic portion comprising at least one elastic material.

According to one embodiment, the elastic material comprises an elastic polymer material.

The elastic polymer material may be an elastic polymer material selected from a list consisting of silicone-based materials and polyurethane-based materials.

According to one embodiment, the first member comprises at least one articulating joint, and the first member is configured to deform by articulation of the at least one articulating joint.

According to one embodiment, the mechanical operation device comprises at least one pulley, and the at least one flexible element is configured to engage the pulley such that transmission is created.

According to one embodiment, a first flexible element is configured to travel from a first point on the second member to the pulley such that the pulley is moved closer to the first point by the first flexible element being pulled in a direction towards the first point, and the transmission created by the pulley causes the pulley to move towards the first point at a lower speed than the first flexible element moves towards the first point.

According to one embodiment, the pulley is connected to the first member.

According to one embodiment, the medical device further comprises at least one second pulley, and the at least one flexible element may be configured to travel from the first to the second pulley such that transmission is created.

According to one embodiment, the mechanical operation device may comprise at least one movable arm having a first portion connected to the first member, and a second portion connected to the second member. The at least one movable arm may comprise at least one operable joint.

According to one embodiment, the at least one movable arm may be configured to deform the first member by moving the first portion of the first member away from the first portion of the second member.

According to one embodiment, the first member is hydraulically operable and may further comprise a hydraulic operation device for operating the first member.

According to one embodiment, the hydraulic operation device comprises at least one hydraulic actuator, directly or indirectly fixated to the first and second members, and wherein the hydraulic actuator is configured to deform the first member by moving the first portion of the first member away from the first portion of the second member.

According to one embodiment, the hydraulic operation device comprises at least one second hydraulic actuator, directly or indirectly fixated to the first and second members, and the second hydraulic actuator may be configured to deform the first member by moving a second portion of the first member away from a second portion of the second member.

According to one embodiment, the medical device may comprise at least one hydraulic conduit for conducting a hydraulic fluid to at least one of the hydraulic actuators.

According to one embodiment, at least one of the first and second members may comprise at least one recess or channel configured to house at least one of the flexible element and the hydraulic conduit.

According to one embodiment, the medical device further comprises at least one sheath configured to house at least one of the: the flexible element, the pulley, the hydraulic conduit and the hydraulic actuator.

According to one embodiment, the sheath comprises elevated and lowered portions, such that the sheath can remain flexible even as fibrotic tissue overgrows the sheath, e.g., at least a portion of the sheath may be shaped like a bellows.

According to one embodiment, the sheath may be connected to the first and second members, such that at least one of the flexible element, the hydraulic conduit, and the hydraulic actuator can travel from the first to the second member through the sheath.

According to one embodiment, the position of the flexible first portion of the first member may be altered by the first member comprising: a first member part forming a closed curve, and an operable second member part having a rigid section and an open or flexible section. The second member part may be displaceable relative to the first member part such that the position of the open or flexible section of the second member part can be altered, for altering the position of the flexible first portion configured to be operated to deform, for changing the stomach wall portion to be stretched between the flexible first portion of the first member and the first portion of the second member.

According to one embodiment, the second member part comprises at least one curvature and may form a circular arc. According to one embodiment, the first member part is ring shaped.

At least one portion of the first member part may be fixedly fixated to the second member and the first member part may comprise a connecting portion for connecting the first member part to the second member.

According to one embodiment, the second member part may be rotatable in relation to the first member part.

According to one embodiment, the second member part is positioned at least partially inside of the first member part.

According to one embodiment, the second member part may comprise teeth configured to be engaged by corresponding teeth of an operation device for operating the second member part.

According to one embodiment, the operation device may comprise a gear wheel or worm wheel for operating the second member part.

According to one embodiment, the rigid section of the second member part forms a semi-circle.

The teeth of the second member part may be positioned on the outer periphery of the rigid section for engaging the gear wheel or worm wheel of the operation device for rotating the second member part inside of the first member part for altering the position of the open or flexible section of the second member part.

According to one embodiment, the first member part comprises elevated and lowered portions, such that the first member part can remain flexible even as fibrotic tissue overgrows the first member part, e.g., at least a portion of the first member part may be shaped like a bellows.

According to one embodiment, the second member is substantially rigid and a major portion of the second member may be made from a material having a modulus of elasticity in the range 50 GPa-400 GPa.

According to one embodiment, the medical device is configured to be completely invaginated by the stomach wall, apart from elements for transferring force or communication to or from the medical device.

According to one embodiment, at least one of the length and the cross-sectional area of at least one of the first and second member is adjustable for calibrating the stretching of the stomach wall.

According to one embodiment, at least one of the length and the cross-sectional area of at least one of the first and second member is mechanically adjustable, for calibrating the stretching of the stomach wall.

The medical device may further comprise a rotatable threaded member for adjusting the length of at least one of the first and second member, for calibrating the stretching of the stomach wall.

At least one of the length and the cross-sectional area of at least one of the first and second member may be hydraulically adjustable, for calibrating the stretching of the stomach wall.

According to one embodiment, at least one of the first and second member may comprise at least one inflatable member, for calibrating the stretching of the stomach wall.

According to one embodiment, at least one of the first and second member may comprise at least one portion comprising elevated and lowered areas for enabling adjustment of at least one of the length and the cross-sectional area of at least one of the first and second member.

According to one embodiment, at least one of the first and second member may comprise at least one bellows-shaped portion for enabling adjustment of at least one of the length and the cross-sectional area of at least one of the first and second member.

According to one embodiment, a portion of the mechanical or hydraulic operation device may be placed in a remote unit configured to be placed at a remote location in the body of the patient, and the medical device may further comprise a force transferring element configured to transfer force hydraulically or mechanically from the remote unit to the first member, for operating the first member.

According to one embodiment, the portion of the operation device placed in the remote unit comprises at least one electrical motor and a gear system configured to reduce the speed and increase the force of the movement generated by the electrical motor.

According to one embodiment, the portion of the operation device placed in the remote unit may comprise at least one pulley for propelling the flexible element.

According to one embodiment, the portion of the operation device placed in the remote unit comprises at least one hydraulic pump. The remote unit may further comprise a hydraulic reservoir for holding a hydraulic fluid.

According to one embodiment, the remote unit comprises an injection port for at least one of injecting and removing hydraulic fluid from the medical device.

According to one embodiment, the remote unit further comprises an energy storage unit for directly or indirectly energizing the medical device.

According to one embodiment, the remote unit further comprises a controller for controlling the operation device.

According to one embodiment, the controller comprises a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

A medical device for actively stretching a stomach wall of a patient for creating a sensation of satiety is provided. The medical device being implantable and comprising an operable first member configured to be fixated to a first portion of the stomach wall, and a second member configured to be fixated to a second portion of the stomach wall, and an operation device. The operation device is configured to pull at least a portion of the first member in a first direction away from at least one portion of the second member, for stretching a portion of the stomach wall between the first portion of the stomach wall and the second portion of the stomach wall for creating the sensation of satiety.

According to one embodiment, the medical device is configured to stretch the stomach wall in at least one main stretching direction, and the operation device is configured to pull at least a portion of the first member in a pulling direction having an angle relative to the main stretching direction of less than 20°.

According to one embodiment, the operation device is configured to displace the first member relative to the second member, such that at least a portion of the first member is moved further away from at least a portion of the second member, for stretching the portion of the stomach wall between the first portion of the stomach wall and the second portion of the stomach wall, for creating the sensation of satiety.

According to one embodiment, the first member is elongated and comprises a first main length axis in the direction of the elongation, and the second member is elongated and comprises a second main length axis in the direction of the elongation. The operation device is configured to displace the first member relative to the second member, such that an angle between the first main length axis and the second main length axis is altered.

According to one embodiment, the operation device is configured to deform the first member, such that at least a portion of the first member is moved further away from at least a portion of the second member, for stretching the portion of the stomach wall between the first portion of the stomach wall and the second portion of the stomach wall, for creating the sensation of satiety.

According to one embodiment, the first member is elongated and comprises a first main length axis in the direction of the elongation, and the second member is elongated and comprises a second main length axis in the direction of the elongation. The operation device is configured to deform the first member, such that an angle between the first main length axis and the second main length axis is altered.

The operation device may be connected to second member such that the connection with the second member provides counterforce against the force needed to pull the at least a portion of the first member in a direction away from at least one portion of the second member.

According to one embodiment, the operation device is further configured to pull at least a portion of the first member in a second direction away from at least one portion of the second member, for stretching a portion of the stomach wall between the first portion of the stomach wall and the second portion of the stomach wall for creating the sensation of satiety, wherein the second direction is different from the first direction.

According to one embodiment, the operation device comprises a first and a second pulling member, and the first pulling member is configured to engage a first portion of the first member and the second pulling member is configured to engage a second portion of the first member, such that: pulling on the first pulling member increases the distance between the first portion of the first member and a first portion of the second member, and pulling on the second pulling member increases the distance between the second portion of the first member and a second portion of the second member.

According to one embodiment, the first pulling member is configured to displace the first portion of the first member relative to the first portion of the second member, and the second pulling member is configured to displace the second portion of the first member relative to the second portion of the second member.

According to one embodiment, the first pulling member is configured to deform the first portion of the first member, and the second pulling member is configured to deform the second portion of the first member.

According to one embodiment, the first member comprises at least one curvature, e.g., at least a portion of the first member forms a circular arc.

According to one embodiment, a portion of the first member is configured to deform, and the portion of the first member configured to deform comprises at least one of the curvature and the circular arc.

According to one embodiment, the second member comprises at least one curvature, e.g., at least a portion of the second member forms a circular arc.

According to one embodiment, the circular are of the first member has a first radius, and the circular are of the second member has a second radius, and wherein the second radius is larger than the first radius.

According to one embodiment, at least one portion of the second member is fixedly fixated to the first member, and the second member may comprise a connecting portion for connecting the second member to the first member.

According to one embodiment, at least two portions of the second member are fixedly fixated to the first member.

According to one embodiment, the footprint of the first member is configured to enclose a portion of the stomach wall, and the footprint of the second member may be configured to enclose a portion of the stomach wall.

According to one embodiment, the footprint of the first member is smaller than the footprint of the second member. The second member may be configured to encircle a portion of the stomach wall of the patient. The second member may comprise a simple closed curve.

According to one embodiment, the second member is ring shaped.

According to one embodiment, the first member is configured to encircle a portion of the stomach wall of the patient and the first member may comprise a simple closed curve, e.g., the first member may be ring shaped.

The first and/or second member may in any of the embodiments be configured to be invaginated by the stomach wall.

According to one embodiment, the first member is configured to be invaginated by the stomach wall.

According to one embodiment, the medical device is configured to be completely invaginated by the stomach wall, apart from elements for transferring force or communication to or from the medical device.

According to one embodiment, the first member is mechanically operable and may comprise a mechanical operation device comprising a pulling member for operating the first member.

According to one embodiment, the pulling member comprises at least one flexible element, directly or indirectly fixated to the first member. The flexible element may be configured to operate the first member by pulling at least a portion of the first member in a first direction away from at least one portion of the second member.

According to one embodiment, a first portion of the flexible element is directly or indirectly fixated to a first portion of the flexible element, and a second portion of the flexible element is directly or indirectly fixated to a second portion of the flexible element. The flexible element may be configured to operate the first member such that the first and second portions are moved in a direction away from at least one portion of the second member.

According to one embodiment, the flexible element is connected to a propulsion device for propelling the flexible element, and propulsion of the flexible element in a first direction causes the first portion of the first member moves away from a first portion of the second member, and propulsion of the flexible element in a second direction causes the second portion of the first member moves away from a second portion of the second member.

According to one embodiment, the mechanical operation device comprises at least one second pulling member comprising at least one second flexible element, directly or indirectly fixated to a second portion of the first member. The second flexible element may be configured to operate the first member such that a second portion of the first member is moved away from a second portion of the second member, for stretching a portion of the stomach wall between the second portion of the first member and the second portion of the second member.

According to one embodiment, the mechanical operation device comprises at least one third pulling member comprising at least one third flexible element, directly or indirectly fixated to a third portion of the first member, and wherein the third flexible element is configured to operate the first member such that a third portion of the first member is moved away from a third portion of the second member, for stretching a portion of the stomach wall between the third portion of the first member and the third portion of the second member.

According to one embodiment, the first member comprises at least one flexible portion, and the first member is configured to deform by deformation of the at least one flexible portion. The first flexible portion may comprise at least one elastic portion which may comprise at least one elastic material which may be an elastic polymer material.

According to one embodiment, the elastic polymer material is an elastic polymer material selected from a list consisting of silicone-based materials and polyurethane-based materials.

According to one embodiment, the first member comprises at least one articulating joint, and the first member may be configured to deform by articulation of the at least one articulating joint.

According to one embodiment, the mechanical operation device may comprise at least one pulley, and the at least one flexible element may be configured to engage the pulley such that transmission is created.

According to one embodiment, a first flexible element may be configured to travel from a first point on the second member to the pulley such that the pulley is moved closer to the first point by the first flexible element being pulled in a direction towards the first point, and the transmission created by the pulley causes the pulley to move towards the first point at a lower speed than the first flexible element moves towards the first point.

According to one embodiment, the pulley is connected to the first member.

The medical device may further comprise at least one second pulley, and the at least one flexible element may be configured to travel from the first to the second pulley such that transmission is created.

According to one embodiment, the mechanical operation device may comprise at least one movable arm having a first portion connected to the first member, and a second portion connected to the second member. The at least one movable arm comprises at least one operable joint or may be configured to deform the first member by moving the first portion of the first member away from the first portion of the second member.

According to one embodiment, the first member is hydraulically operable.

According to one embodiment, the medical device further comprises a hydraulic operation device comprising a pulling member for operating the first member.

According to one embodiment, the hydraulic operation device comprises at least one pulling member comprising at least one hydraulic actuator, directly or indirectly fixated to the first and second members. The hydraulic actuator is configured to move the first member by pulling the first portion of the first member in a first direction away from the first portion of the second member.

According to one embodiment, the hydraulic operation device comprises at least one second pulling member comprising at least one second hydraulic actuator, directly or indirectly fixated to the first and second members. The second hydraulic actuator is configured to move the first member by moving a second portion of the first member away from a second portion of the second member.

According to one embodiment, the medical device comprises at least one hydraulic conduit for conducting a hydraulic fluid to at least one of the hydraulic actuators.

According to one embodiment, at least one of the first and second members comprises at least one recess or channel configured to house at least one of the flexible element and the hydraulic conduit.

According to one embodiment, the medical device may further comprise at least one sheath configured to house at least one of the: the flexible element, the pulley, the hydraulic conduit and the hydraulic actuator.

According to one embodiment, the sheath comprises elevated and lowered portions, such that the sheath can remain flexible even as fibrotic tissue overgrows the sheath. E.g., at least a portion of the sheath may be shaped like a bellows.

According to one embodiment, the sheath is connected to the first and second members, such that at least one of the flexible element, the hydraulic conduit, and the hydraulic actuator can travel from the first to the second member through the sheath.

According to one embodiment, the position of the portion of the first member configured to be pulled can be altered by the first member comprising: a first member part forming a closed curve, and an operable second member part having a rigid section and an open or flexible section. The second member part may be displaceable relative to the first member part such that the position of the open or flexible section of the second member part can be altered, for altering the position of the portion of the first member configured to be pulled, for changing the stomach wall portion to be stretched between the portion of the first member configured to be pulled and a portion of the second member.

According to one embodiment, the second member part comprises at least one curvature.

According to one embodiment, at least a portion of the second member part forms a circular arc, and the first member part may be ring-shaped.

According to one embodiment, at least one portion of the first member part may be fixedly fixated to the second member. The first member part may comprise a connecting portion for connecting the first member part to the second member.

The second member part may be rotatable in relation to the first member part.

According to one embodiment, the second member part may be positioned at least partially inside of the first member part.

According to one embodiment, second member part comprises teeth configured to be engaged by corresponding teeth of an operation device for operating the second member part.

According to one embodiment, the operation device comprises a gear wheel or worm wheel for operating the second member part.

According to one embodiment, the rigid section of the second member part may form a semi-circle.

According to one embodiment, the teeth of the second member part are positioned on the outer periphery of the rigid section for engaging the gear wheel or worm wheel of the operation device for rotating the second member part inside of the first member part for altering the position of the open or flexible section of the second member part.

According to one embodiment, the first member part may comprise elevated and lowered portions, such that the first member part can remain flexible even as fibrotic tissue overgrows the first member part, e.g., at least a portion of the first member part is shaped like a bellows.

According to one embodiment, the second member is substantially rigid, and a major portion of the second member may be made from a material having a modulus of elasticity in the range 50 GPa-400 GPa.

According to one embodiment, at least one of the length and the cross-sectional area of at least one of the first and second member may be adjustable for calibrating the stretching of the stomach wall.

According to one embodiment, the length and/or the cross-sectional area of at least one of the first and second member may be mechanically adjustable, for calibrating the stretching of the stomach wall.

According to one embodiment, the medical device further comprises a rotatable threaded member for adjusting the length of at least one of the first and second member, for calibrating the stretching of the stomach wall.

According to one embodiment, at least one of the length and the cross-sectional area of at least one of the first and second member is hydraulically adjustable, for calibrating the stretching of the stomach wall.

According to one embodiment, at least one of the first and second member comprises at least one inflatable member, for calibrating the stretching of the stomach wall.

According to one embodiment, at least one of the first and second members comprises at least one portion comprising elevated and lowered areas for enabling adjustment of at least one of the length and the cross-sectional area of at least one of the first and second member.

According to one embodiment, at least one of the first and second members comprises at least one bellows-shaped portion for enabling adjustment of at least one of the length and the cross-sectional area of at least one of the first and second member.

According to one embodiment, a portion of the mechanical or hydraulic operation device may be placed in a remote unit configured to be placed at a remote location in the body of the patient, and the medical device may further comprise a force transferring element configured to transfer force hydraulically or mechanically from the remote unit to the first member, for operating the first member.

According to one embodiment, the portion of the operation device placed in the remote unit comprises at least one electrical motor and a gear system configured to reduce the speed and increase the force of the movement generated by the electrical motor.

According to one embodiment, the portion of the operation device placed in the remote unit may comprise at least one pulley for propelling the flexible element.

According to one embodiment, the portion of the operation device placed in the remote unit comprises at least one hydraulic pump. The remote unit may further comprise a hydraulic reservoir for holding a hydraulic fluid.

According to one embodiment, the remote unit comprises an injection port for at least one of injecting and removing hydraulic fluid from the medical device.

According to one embodiment, the remote unit further comprises an energy storage unit for directly or indirectly energizing the medical device.

According to one embodiment, the remote unit further comprises a controller for controlling the operation device.

According to one embodiment, the controller comprises a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

A medical device for actively stretching a stomach wall of a patient for creating a sensation of satiety. The medical device being implantable and comprising an operable first member configured to be fixated to a first portion of the stomach wall and a second member configured to be fixated to a second portion of the stomach wall. The medical device further comprises a mechanical operation device configured to move at least a portion of the first member in a first direction away from at least one portion of the second member, for stretching a portion of the stomach wall between the first portion of the stomach wall and the second portion of the stomach wall, for creating the sensation of satiety. A portion of the operation device is placed in a remote unit configured to be placed at a remote location in the body of the patient and the medical device further comprises a force transferring element configured to transfer mechanical force from the remote unit to the first member, for operating the first member.

According to one embodiment, the medical device is configured to stretch the stomach wall in at least one main stretching direction, and the operation device is configured to pull at least a portion of the first member in a pulling direction having an angle relative to the main stretching direction of less than 20°.

According to one embodiment, the operation device is configured to displace the first member relative to the second member, such that at least a portion of the first member is moved further away from at least a portion of the second member, for stretching the portion of the stomach wall between the first portion of the stomach wall and the second portion of the stomach wall, for creating the sensation of satiety.

The first member may be elongated and comprise a first main length axis in the direction of the elongation, and the second member may be elongated and comprise a second main length axis in the direction of the elongation. The operation device may be configured to displace the first member relative to the second member, such that an angle between the first main length axis and the second main length axis is altered.

According to one embodiment, the operation device is configured to deform the first member, such that at least a portion of the first member is moved further away from at least a portion of the second member, for stretching the portion of the stomach wall between the first portion of the stomach wall and the second portion of the stomach wall, for creating the sensation of satiety.

According to one embodiment, the first member is elongated and comprises a first main length axis in the direction of the elongation, and the second member is elongated and comprises a second main length axis in the direction of the elongation. The operation device is configured to deform the first member, such that an angle between the first main length axis and the second main length axis is altered.

According to one embodiment, the operation device is connected to second member such that the connection with the second member provides counterforce against the force needed to move the at least a portion of the first member in a direction away from at least one portion of the second member.

According to one embodiment, the operation device is further configured to move at least a portion of the first member in a second direction away from at least one portion of the second member, for stretching a portion of the stomach wall between the first portion of the stomach wall and the second portion of the stomach wall for creating the sensation of satiety, wherein the second direction is different from the first direction.

According to one embodiment, the operation device comprises a first and a second operating member, and the first operating member is configured to engage a first portion of the first member and the second operating member is configured to engage a second portion of the first member, such that operation of the first operating member increases the distance between the first portion of the first member and a first portion of the second member, and operation of the second operating member increases the distance between the second portion of the first member and a second portion of the second member.

According to one embodiment, the first operating member is configured to displace the first portion of the first member relative to the first portion of the second member, and the second operating member is configured to displace the second portion of the first member relative to the second portion of the second member.

According to one embodiment, the first operating member is configured to deform the first portion of the first member, and the second operating member is configured to deform the second portion of the first member.

The first member may comprise at least one curvature and at least a portion of the first member may form a circular arc.

According to one embodiment, a portion of the first member is configured to deform, and the portion of the first member configured to deform may comprise at least one of the curvature and the circular arc.

The second member may comprise at least one curvature, and at least a portion of the second member may form a circular arc.

According to one embodiment, the circular are of the first member has a first radius, and the circular are of the second member has a second radius, and the second radius is larger than the first radius.

According to one embodiment, at least one portion of the second member is fixedly fixated to the first member, and the second member may comprise a connecting portion for connecting the second member to the first member.

According to one embodiment, at least two portions of the second member may be fixedly fixated to the first member.

The footprint of the first member may be configured to enclose a portion of the stomach wall.

According to one embodiment, the footprint of the second member may be configured to enclose a portion of the stomach wall, and the footprint of the first member may be smaller than the footprint of the second member.

According to one embodiment, the second member may be configured to encircle a portion of the stomach wall of the patient, and the second member may comprise a simple closed curve.

E.g., the second member may be ring-shaped.

According to one embodiment, the first member may be configured to encircle a portion of the stomach wall of the patient and the first member may comprise a simple closed curve. E.g., the first member may be ring-shaped.

According to one embodiment, the first member comprises at least one flexible portion, and the first member may be configured to deform by deformation of the at least one flexible portion.

The first flexible portion may comprise at least one elastic portion, which may comprises at least one elastic material. The elastic material may comprise an elastic polymer material, which may be an elastic polymer material selected from a list consisting of silicone-based materials and polyurethane-based materials.

According to one embodiment, the first member comprises at least one articulating joint, and the first member may be configured to deform by articulation of the at least one articulating joint.

In any of the embodiments herein, the first and/or second members may be fixated to the stomach wall by means of at least partial invagination.

According to one embodiment, the medical device is configured to be completely invaginated by the stomach wall, apart from elements for transferring force or communication to or from the medical device.

According to one embodiment, the first and second members are parts of a main portion placed in proximity to the stomach wall, and a portion of the operation device is placed in the main portion, and the portion of the operation device placed in the main portion comprises a receiving portion configured to receive mechanical force from a remote unit. The main portion may comprise a transmission for transforming the received mechanical force into a force for operating the first member for stretching the stomach wall. The transmission may comprise a gear system configured to reduce the speed and increase the force of the received mechanical force.

According to one embodiment, the mechanical operation device comprises at least one flexible element, directly or indirectly fixated to the first member. The flexible element may be configured to operate the first member to move at least a portion of the first member in a first direction away from at least one portion of the second member.

According to one embodiment, the main portion comprises at least one pulley, and the at least one flexible element may be configured to engage the pulley such that transmission is created.

According to one embodiment, the transmission comprises a first and second pulley, a flexible element configured to be placed around the first and a second pulley. The flexible element may be configured to be pulled by the force having the first speed and first strength, causing the first and second pulley to displace in relation to each other with the force having the second lower speed and the second higher strength, thereby creating the transmission.

According to one embodiment, a first flexible element is configured to travel from a first point on the second member to the pulley such that the pulley is moved closer to the first point by the first flexible element being pulled in a direction towards the first point, and the transmission created by the pulley causes the pulley to move towards the first point at a lower speed than the first flexible element moves towards the first point. At least one pulley may be connected to the first member.

According to one embodiment, the receiving portion may be configured to receive a rotating mechanical force, and the transmission is configured to transform the received rotating mechanical force into a liner mechanical force.

According to one embodiment, the receiving portion is configured to receive a linear mechanical force.

According to one embodiment, the main portion comprises at least one operating member in the form of a pulling member configured to pull at least a portion of the first member in a first direction away from at least one portion of the second member.

The pulling member may comprise a flexible element which may be a flexible wire, such as a nylon wire.

According to one embodiment, a first portion of the flexible element is directly or indirectly fixated to a first portion of the flexible element, and a second portion of the flexible element is directly or indirectly fixated to a second portion of the flexible element, and the flexible element may be configured to operate the first member such that the first and second portion is moved away from at least one portion of the second member.

According to one embodiment, the flexible element is connected to a propulsion device for propelling the flexible element, and propulsion of the flexible element in a first direction may cause the first member to deform such that the first portion of the first member moves away from a first portion of the second member, and propulsion of the flexible element in a second direction may cause the first member to deform such that the second portion of the first member moves away from a second portion of the second member.

According to one embodiment, the mechanical operation device may comprise at least one second flexible element, directly or indirectly fixated to a second portion of the first member, and the second flexible element may be configured to operate the first member such that a second portion of the first member is moved away from a second portion of the second member, for stretching a portion of the stomach wall between the second portion of the first member and the second portion of the second member.

According to one embodiment, the mechanical operation device may comprise at least one third pulling member comprising at least one third flexible element, directly or indirectly fixated to a third portion of the first member. The third flexible element may be configured to operate the first member such that a third portion of the first member is moved away from a third portion of the second member, for stretching a portion of the stomach wall between the third portion of the first member and the third portion of the second member.

According to one embodiment, the mechanical operation device may comprise at least one movable arm having a first portion connected to the first member, and a second portion connected to the second member. The at least one movable arm may comprise at least one operable joint.

According to one embodiment, the at least one movable arm may be configured to deform the first member by moving the first portion of the first member away from the first portion of the second member.

The main portion may comprise at least one recess or channel configured to house the flexible element.

According to one embodiment, the main portion may comprise at least one sheath configured to house at least one of the flexible element and the pulley.

According to one embodiment, the sheath comprises elevated and lowered portions, such that the sheath can remain flexible even as fibrotic tissue overgrows the sheath. E.g., the sheath may be shaped like a bellows.

According to one embodiment, the sheath may be connected to the first and second members, such that the flexible element can travel from the first to the second member through the sheath.

According to one embodiment, the second member is substantially rigid, which may mean that a major portion of the second member is made from a material having a modulus of elasticity in the range 50 GPa-400 GPa.

According to one embodiment, at least one of the length and the cross-sectional area of at least one of the first and second member is adjustable for calibrating the stretching of the stomach wall.

The length and/or the cross-sectional area of at least one of the first and second member may be mechanically adjustable, for calibrating the stretching of the stomach wall.

According to one embodiment, the medical device further comprises a rotatable threaded member for adjusting the length of at least one of the first and second member, for calibrating the stretching of the stomach wall.

According to one embodiment, at least one of the length and the cross-sectional area of at least one of the first and second member is hydraulically adjustable, for calibrating the stretching of the stomach wall.

According to one embodiment, at least one of the first and second member comprises at least one inflatable member, for calibrating the stretching of the stomach wall.

At least one of the first and second members may comprise at least one portion comprising elevated and lowered areas for enabling adjustment of at least one of the length and the cross-sectional area of at least one of the first and second member.

According to one embodiment, at least one of the first and second members comprises at least one bellows-shaped portion for enabling adjustment of at least one of the length and the cross-sectional area of at least one of the first and second member.

According to one embodiment, the portion of the operation device placed in the remote unit may comprise at least one electrical motor.

The operation device placed in the remote unit may further comprise a gear system configured to reduce the speed and increase the force of the movement generated by the electrical motor.

According to one embodiment, the portion of the operation device placed in the remote unit may comprise at least one pulley for propelling the flexible element.

According to one embodiment, the remote unit may further comprise an energy storage unit for directly or indirectly energizing the medical device.

According to one embodiment, the remote unit may further comprise a controller for controlling the operation device. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach wall of a patient for creating a sensation of satiety is provided. The medical device is implantable and comprises a first member, a second member and an operation device. The first member comprises a first member rotating section, configured to rotate around a first axis, and a first member fixation section coupled to the first member rotating section and configured to be fixated to a first portion of the stomach wall. The second member is coupled to the first member rotating section, and comprises a second member fixation section configured to be fixated to a second portion of the stomach wall. The operation device is coupled to the first member rotating section, and is configured to induce a rotation of the first member rotating section around the first axis. The first member fixation section is configured to describe a revolution around the first axis between a first position of the first member fixation section and a second position of the first member fixation section in response to the rotation of the first member rotating section. Each of the first member rotating section and the operation device is positioned below a first reference plane perpendicular to the first axis, and the first member fixation section is positioned above said first reference plane for at least a part of the revolution of the first member fixation section. The stomach wall between the first portion and second portion is increasingly stretched when the first member fixation section revolves towards the second position of the first member fixation section.

The rotation of the first member rotating section around the first axis is advantageous because, when a fibrotic tissue has encapsulated the first member rotating section, stretching the stomach wall between the first portion and second portion does not require bending of the fibrotic tissue. This is important because the fibrotic tissue is substantially inelastic, thus being not easily bendable. The first member rotating section may easily rotate around the first axis while encapsulated by the fibrotic tissue, which does not hinder the rotation.

According to one embodiment, the first member rotating section is configured to be at least partially invaginated by a tissue of the stomach wall. Invagination improves stability and long term fixation of the medical device. The first member rotating section and first member fixation section may be a rod with a circular cross-section area. The first member may further comprise a first member coupling section connecting the first member rotating section and the first member fixation section. The first member coupling section may have a circular cross-section area. The first member coupling section may be positioned above the first reference plane. The first member coupling section may be a rod arranged at angle in relation to the first axis. The first member fixation section may be parallel to the first member rotating section.

According to one embodiment, the medical device further comprises a main portion, and the operation device comprises a local portion placed in the main portion. The main portion may be configured to be at least partially invaginated by a tissue of the stomach wall. Invagination improves stability and long term fixation of the medical device.

According to one embodiment, the first member fixation section is further configured to describe the revolution around the first axis maintaining a fixed distance from the first axis.

According to one embodiment, the local portion of the operation device comprises a first conversion device. The first conversion device comprises a receiving portion configured to receive an input local force, and a transmission portion coupled to the receiving portion and to the first member rotating section, the transmission portion being configured to transform the input local force into an output local force inducing a rotation of the first member rotating section around the first axis. The receiving portion may be arranged at a distance from the transmission portion, said distance being a characteristic distance of the first conversion device. By reducing the characteristic distance of the first conversion device as compared to the distance of the first member fixation from the first axis, a smaller input local force needs to be applied in order for the first member fixation section to stretch the stomach wall with a given stretching force. Likewise, by reducing the characteristic distance of the first conversion device as compared to the distance of the first member fixation from the first axis, a larger input local speed is required in order for the first member fixation section to stretch the stomach wall with a given stretching speed.

According to one embodiment, the first conversion device is a first pulley. The first pulley has a rim, configured to engage a flexible wire, where the rim is the receiving portion of the first conversion device. The first pulley also has a central portion, wherein the central portion is the transmission portion of the first conversion device.

According to one embodiment, the rim of the first pulley is circular and the first pulley has a radius, the radius of the first pulley being the characteristic distance of the first pulley. The characteristic distance of the first conversion device may be in a range of 0.3 times to 2 times as compared to the distance of the first member fixation section from the first axis. The characteristic distance of the first conversion device may be in a range of 0.5 times to 1.2 times as compared to the distance of the first member fixation section from the first axis. The characteristic distance of the first conversion device may be in a range of 0.95 times to 1.05 times as compared to the distance of the first member fixation section from the first axis.

According to one embodiment, the operation device comprises a first flexible wire configured to engage with the rim of the first pulley to induce a rotation of the first pulley.

According to one embodiment, the first flexible wire configured to engage with the rim of the first pulley is further configured to pull the first pulley in a first rotating direction of the first pulley, and the medical device further comprises a first spring mechanism coupled to the first pulley and configured to bias the first pulley towards a second rotating direction of the first pulley opposite to the first rotating direction of the first pulley.

According to one embodiment, the first flexible wire configured to engage with the rim of the first pulley is further configured to pull the first pulley in a first rotating direction of the first pulley, and the medical device further comprises a second flexible wire configured to engage with the rim of the first pulley to pull the first pulley in a second rotating direction of the first pulley opposite to the first rotating direction of the first pulley.

According to one embodiment, the local portion of the operation device further comprises a first local motor coupled to the receiving portion of the first conversion device, and the first local motor is configured to generate a movement having a first input local force and a first input local speed. The first local motor may be a piezoelectric motor. The first conversion device may be a gear system configured to reduce the first input local speed and increase the first input local force.

According to one embodiment, the medical device further comprises a remote unit configured to be placed at a remote location in the body of the patient, and the operation device further comprises a remote portion placed in the remote unit.

According to one embodiment, the remote portion of the operation device further comprises a first remote motor coupled to the receiving portion of the first conversion device and configured to generate a movement having a first input remote speed and a first input remote force. The first remote motor may be a piezoelectric motor. The remote portion of the operation device may further comprise a first remote gear system configured to reduce the first input remote speed and increase the first input remote force. The remote portion of the operation device may further comprise a second remote motor coupled to the receiving portion of the first conversion device and configured to generate a movement having a second input remote speed and a second input remote force. The second remote motor may be a piezoelectric motor. The remote portion of the operation device may further comprise a second remote gear system configured to reduce the second input remote speed and increase the second input remote force.

According to one embodiment, the operation device further comprises a first flexible wire configured to transmit the first input remote force from the first remote motor to the receiving portion of the first conversion device.

According to one embodiment, the operation device further comprises a second flexible wire configured to transmit the second input remote force from the second remote motor to the receiving portion of the first conversion device, wherein the first flexible wire is configured to induce a rotation of the first member rotating section in a first rotating direction of the first member rotating section, and the second flexible wire is configured to induce a rotation of the first member rotating section in a second rotating direction of the first member rotating section opposite to the first rotating direction of the first member rotating section.

According to one embodiment, the operation device further comprises a first hydraulic system configured to transmit the first input remote force from the first remote motor to the receiving portion of the first conversion device. The first hydraulic system may comprise a first hydraulic conduit, a first remote hydraulic cylinder and a first local hydraulic cylinder. The first remote hydraulic cylinder is placed in the remote portion of the operation device, and is coupled to the first remote motor and to the first hydraulic conduit. The first local hydraulic cylinder is placed in the local portion of the operation device, and is coupled to the first hydraulic conduit and to the receiving portion of the first conversion device.

According to one embodiment, the operation device further comprises a second hydraulic system configured to transmit the second input remote force from the second remote motor to the receiving portion of the first conversion device. The first hydraulic system is configured to induce a rotation of the first member rotating section in a first rotating direction of the first member rotating section, and the second hydraulic system is configured to induce a rotation of the first member rotating section in a second rotating direction of the first member rotating section opposite to the first rotating direction of the first member rotating section. The second hydraulic system may further comprise a second hydraulic conduit, a second remote hydraulic cylinder and a second local hydraulic cylinder. The second remote hydraulic cylinder is placed in the remote portion of the operation device, and is coupled to the second remote motor and to the second hydraulic conduit. The second local hydraulic cylinder is placed in the local portion of the operation device, and is coupled to the second hydraulic conduit and to the receiving portion of the first conversion device.

According to one embodiment, the second member is inoperably fixated to the main portion. The second member may be configured to be at least partially invaginated by a tissue of the stomach wall. Invagination improves stability and long term fixation of the medical device. The second member may be a rod having a circular cross-section area. The second member may be parallel to the first member fixation section. This is advantageous in that the stretching of the stomach wall between the first portion and second portion is uniform.

According to one embodiment, the medical device further comprises a third member coupled to the first member rotating section, the third member comprising a third member fixation section configured to be fixated to a third portion of the stomach wall. The stomach wall between the first portion and third portion is increasingly stretched when the first member fixation section revolves towards the first position of the first member fixation section.

According to one embodiment, the third member is inoperably fixated to the main portion.

The third member may be configured to be at least partially invaginated by a tissue of the stomach wall.

Invagination improves stability and long term fixation of the medical device. The third member may be a rod having a circular cross-section area. The third member may be parallel to the first member fixation section. This is advantageous in that the stretching of the stomach wall between the first portion and third portion is uniform.

According to one embodiment, the second member further comprises a second member rotating section and a second member fixation section. The second member rotating section is coupled to the first member rotating section and configured to rotate around a second axis. The second member fixation section is coupled to the second member rotating section and configured to be fixated to the second portion of the stomach wall. The operation device is further coupled to the second member rotating section and further configured to induce a rotation of the second member rotating section around the second axis. The second member fixation section is configured to describe a revolution around the second axis between a first position of the second member fixation section and a second position of the second member fixation section in response to the rotation of the second member rotating section. Each of the second member rotating section and the operation device is positioned below a second reference plane perpendicular to the second axis. The second member fixation section is positioned above said second reference plane for at least a part of the revolution of the second member fixation section. The stomach wall between the first portion and second portion is increasingly stretched when the second member fixation section revolves towards the first position of the second member fixation section.

The rotation of the second member rotating section around the second axis is advantageous because, when a fibrotic tissue has encapsulated the second member rotating section, stretching the stomach wall between the first portion and second portion does not require bending of the fibrotic tissue. This is important because the fibrotic tissue is substantially inelastic, thus being not easily bendable. The second member rotating section may easily rotate around the second axis while encapsulated by the fibrotic tissue, which does not hinder the rotation.

According to one embodiment, the second member rotating section is configured to be at least partially invaginated by a tissue of the stomach wall. Invagination improves stability and long term fixation of the medical device. At least one of the second member rotating section and second member fixation section may be a rod with a circular cross-section area. The second member may further comprise a second member coupling section connecting the second member rotating section and the second member fixation section. The second member coupling section may have a circular cross-section area. The second member coupling section may be positioned above the second reference plane. The second member coupling section may be a rod arranged at angle in relation to the second axis. The second member fixation section may be parallel to the second member rotating section. The second member fixation section may be parallel to the first member fixation section. This is advantageous in that the stretching of the stomach wall between the first portion and second portion is uniform. The second axis may be parallel to the first axis.

According to one embodiment, the second member fixation section is further configured to describe the revolution around the second axis maintaining a fixed distance from the second axis.

According to one embodiment, the local portion of the operation device comprises a second conversion device, the second conversion device comprising a receiving portion and a transmission portion. The receiving portion is configured to receive an input local force. The transmission portion is coupled to the receiving portion of the second conversion device and to the second member rotating section. The transmission portion of the second conversion device is configured to transform the input local force received by the receiving portion of the second conversion device into an output local force inducing a rotation of the second member rotating section around the second axis.

The receiving portion of the second conversion device may be arranged at a distance from the transmission portion of the second conversion device, said distance being a characteristic distance of the second conversion device. By reducing the characteristic distance of the second conversion device as compared to the distance of the second member fixation section from the second axis, a smaller input local force needs to be applied in order for the second member fixation section to stretch the stomach wall with a given stretching force. Likewise, by reducing the characteristic distance of the second conversion device as compared to the distance of the second member fixation section from the second axis, a larger input local speed is required in order for the second member fixation section to stretch the stomach wall with a given stretching speed.

According to one embodiment, the second conversion device is a second pulley, the second pulley having a rim, where the rim of the second pulley is the receiving portion of the second conversion device, and a central portion, where the central portion of the second pulley is the transmission portion of the second conversion device. The rim is configured to engage a flexible wire.

According to one embodiment, the rim of the second pulley is circular and the second pulley has a radius, the radius of the second pulley being the characteristic distance of the second pulley.

The characteristic distance of the second conversion device may be in a range of 0.3 times to 2 times as compared to the distance of the second member fixation section from the second axis. The characteristic distance of the second conversion device may be in a range of 0.5 times to 1.2 times as compared to the distance of the second member fixation section from the second axis. The characteristic distance of the second conversion device may be in a range of 0.95 times to 1.05 times as compared to the distance of the second member fixation section from the second axis.

According to one embodiment, the operation device comprises a third flexible wire configured to engage with the rim of the second pulley to induce a rotation of the second pulley. The third flexible wire configured to engage with the rim of the second pulley is further configured to pull the second pulley in a first rotating direction of the second pulley. The medical device further comprises a second spring mechanism coupled to the second pulley and configured to bias the second pulley towards a second rotating direction of the second pulley opposite to the first rotating direction of the second pulley.

According to one embodiment, the third flexible wire configured to engage with the rim of the second pulley is further configured to pull the second pulley in a first rotating direction of the second pulley. The medical device further comprises a fourth flexible wire configured to engage with the rim of the second pulley to pull the second pulley in a second rotating direction of the second pulley opposite to the first rotating direction of the second pulley.

According to one embodiment, the first flexible wire configured to engage with the rim of the first pulley is the same as the third flexible wire configured to engage with the rim of the second pulley, and is configured to pull the first pulley and the second pulley in opposite rotating directions.

According to one embodiment, the first flexible wire configured to engage with the rim of the first pulley is the same as the third flexible wire configured to engage with the rim of the second pulley, the second flexible wire configured to engage with the rim of the first pulley is the same as the fourth flexible wire configured to engage with the rim of the second pulley, the first rotating direction of the first pulley is opposite to the first rotating direction of the second pulley, and the second rotating direction of the first pulley is opposite to the second rotating direction of the second pulley.

According to one embodiment, the local portion of the operation device further comprises a second local motor coupled to the receiving portion of the second conversion device, and the second local motor is configured to generate a movement having a second input local force and a second input local speed. The second local motor may be a piezoelectric motor. The second conversion device may be a gear system configured to reduce the second input local speed and increase the second input local force.

According to one embodiment, the remote portion of the operation device further comprises a third remote motor coupled to the receiving portion of the second conversion device and configured to generate a movement having a third input remote speed and a third input remote force. The third remote motor may be a piezoelectric motor. The remote portion of the operation device may further comprise a third remote gear system configured to reduce the third input remote speed and increase the third input remote force.

According to one embodiment, the remote portion of the operation device further comprises a fourth remote motor coupled to the receiving portion of the second conversion device and configured to generate a movement having a fourth input remote speed and a fourth input remote force.

The fourth remote motor may be a piezoelectric motor. The remote portion of the operation device may further comprise a fourth remote gear system configured to reduce the fourth input remote speed and increase the fourth input remote force.

According to one embodiment, the operation device further comprises a third flexible wire configured to transmit the third input remote force from the third remote motor to the receiving portion of the second conversion device.

According to one embodiment, the operation device further comprises a fourth flexible wire configured to transmit the fourth input remote force from the fourth remote motor to the receiving portion of the second conversion device, wherein the third flexible wire is configured to induce a rotation of the second member rotating section in a first rotating direction of the second member rotating section, and the fourth flexible wire is configured to induce a rotation of the second member rotating section in a second rotating direction of the second member rotating section opposite to the first rotating direction of the second member rotating section.

According to one embodiment, the first flexible wire is the same as the third flexible wire, the first remote motor is the same as the third remote motor, and the first flexible wire is configured to rotate the first member rotating section and second member rotating section in opposite rotating directions.

According to one embodiment, the first flexible wire is the same as the third flexible wire, the first remote motor is the same as the third remote motor, the second flexible wire is the same as the fourth flexible wire and the second remote motor is the same as the fourth remote motor. The first rotating direction of the first member rotating section is opposite to the first rotating direction of the second member rotating section, and the second rotating direction of the first member rotating section is opposite to the second rotating direction of the second member rotating section.

According to one embodiment, the operation device further comprises a third hydraulic system configured to transmit the third input remote force from the third remote motor to the receiving portion of the second conversion device. The third hydraulic system may comprise a third hydraulic conduit, a third remote hydraulic cylinder and a third local hydraulic cylinder. The third remote hydraulic cylinder is placed in the remote portion of the operation device, and is coupled to the third remote motor and to the third hydraulic conduit. The third local hydraulic cylinder is placed in the local portion of the operation device, and is coupled to the third hydraulic conduit and to the receiving portion of the second conversion device.

According to one embodiment, the operation device further comprises a fourth hydraulic system configured to transmit the fourth input remote force from the fourth remote motor to the receiving portion of the second conversion device. The third hydraulic system is configured to induce a rotation of the second member rotating section in a first rotating direction of the second member rotating section, and the fourth hydraulic system is configured to induce a rotation of the second member rotating section in a second rotating direction of the second member rotating section opposite to the first rotating direction of the second member rotating section. The fourth hydraulic system may comprise a fourth hydraulic conduit, a fourth remote hydraulic cylinder and a fourth local hydraulic cylinder. The fourth remote hydraulic cylinder is placed in the remote portion of the operation device, and is coupled to the fourth remote motor and to the fourth hydraulic conduit. The fourth local hydraulic cylinder is placed in the local portion of the operation device, and is coupled to the fourth hydraulic conduit and to the receiving portion of the second conversion device.

According to one embodiment, the first hydraulic system is the same as the third hydraulic system, the first remote motor is the same as the third remote motor, and the first hydraulic system is configured to rotate the first member rotating section and second member rotating section in opposite rotating directions.

According to one embodiment, the first hydraulic system is the same as the third hydraulic system, the first remote motor is the same as the third remote motor, the second hydraulic system is the same as the fourth hydraulic system and the second remote motor is the same as the fourth remote motor.

The first rotating direction of the first member rotating section is opposite to the first rotating direction of the second member rotating section, and the second rotating direction of the first member rotating section is opposite to the second rotating direction of the second member rotating section.

According to one embodiment, the medical device further comprises a third member, the third member comprising a third member rotating section and a third member fixation section. The third member rotating section is coupled to the second member and configured to rotate around a third axis.

The third member fixation section is coupled to the third member rotating section and configured to be fixated to a third portion of the stomach wall. The operation device is further coupled to the third member rotating section and further configured to induce a rotation of the third member rotating section around the third axis. The third member fixation section is configured to describe a revolution around the third axis between a first position of the third member fixation section and a second position of the third member fixation section in response to the rotation of the third member rotating section. Each of the third member rotating section and the operation device is positioned below a third reference plane perpendicular to the third axis, and the third member fixation section is positioned above said third reference plane for at least a part of the revolution of the third member fixation section. The stomach wall between the second portion and third portion is increasingly stretched when the third member fixation section revolves towards the first position of the third member.

The rotation of the third member rotating section around the third axis is advantageous because, when a fibrotic tissue has encapsulated the third member rotating section, stretching the stomach wall between the second portion and third portion does not require bending of the fibrotic tissue. This is important because the fibrotic tissue is substantially inelastic, thus being not easily bendable. The third member rotating section may easily rotate around the third axis while encapsulated by the fibrotic tissue, which does not hinder the rotation.

According to one embodiment, the third member rotating section is configured to be at least partially invaginated by a tissue of the stomach wall. Invagination improves stability and long term fixation of the medical device. At least one of the third member rotating section and third member fixation section may be a rod with a circular cross-section area. The third member may further comprise a third member coupling section connecting the third member rotating section and the third member fixation section. The third member coupling section may have a circular cross-section area. The third member coupling section may be positioned above the third reference plane. The third member coupling section may be a rod arranged at angle in relation to the third axis. The third member fixation section may be parallel to the third member rotating section. The third axis may be parallel to the first axis. This is advantageous in that the stretching of the stomach wall between the third portion and second portion is uniform.

According to one embodiment, the third member fixation section is further configured to describe the revolution around the third axis maintaining a fixed distance from the third axis.

According to one embodiment, the local portion of the operation device comprises a second conversion device, the second conversion device comprising a receiving portion and a transmission portion. The receiving portion is configured to receive an input local force. The transmission portion is coupled to the receiving portion of the second conversion device and to the third member rotating section. The transmission portion of the second conversion device is configured to transform the input local force received by the receiving portion of the second conversion device into an output local force inducing a rotation of the third member rotating section around the third axis. The receiving portion of the second conversion device may be arranged at a distance from the transmission portion of the second conversion device, said distance being a characteristic distance of the second conversion device. By reducing the characteristic distance of the second conversion device as compared to the distance of the third member fixation section from the third axis, a smaller input local force needs to be applied in order for the third member fixation section to stretch the stomach wall with a given stretching force. Likewise, by reducing the characteristic distance of the second conversion device as compared to the distance of the third member fixation section from the third axis, a larger input local speed is required in order for the third member fixation section to stretch the stomach wall with a given stretching speed.

According to one embodiment, the second conversion device is a second pulley, the second pulley having a rim and a central portion. The rim is the receiving portion of the second conversion device and is configured to engage a flexible wire. The central portion is the transmission portion of the second conversion device.

According to one embodiment, the rim of the second pulley is circular and the second pulley has a radius, the radius of the second pulley being the characteristic distance of the second pulley.

The characteristic distance of the second conversion device may be in a range of 0.3 times to 2 times as compared to the distance of the third member fixation section from the third axis. The characteristic distance of the second conversion device may be in a range of 0.5 times to 1.2 times as compared to the distance of the third member fixation section from the third axis. The characteristic distance of the second conversion device may be in a range of 0.95 times to 1.05 times as compared to the distance of the third member fixation section from the third axis.

According to one embodiment, the operation device comprises a third flexible wire configured to engage with the rim of the second pulley to induce a rotation of the second pulley.

According to one embodiment, the third flexible wire configured to engage with the rim of the second pulley is further configured to pull the second pulley in a first rotating direction of the second pulley, and the medical device further comprises a second spring mechanism coupled to the second pulley and configured to bias the second pulley towards a second rotating direction of the second pulley opposite to the first rotating direction of the second pulley.

According to one embodiment, the third flexible wire configured to engage with the rim of the second pulley is further configured to pull the second pulley in a first rotating direction of the second pulley, and the medical device further comprises a fourth flexible wire configured to engage with the rim of the second pulley to pull the second pulley in a second rotating direction of the second pulley opposite to the first rotating direction of the second pulley.

According to one embodiment, the first flexible wire configured to engage with the rim of the first pulley is the same as the third flexible wire configured to engage with the rim of the second pulley, and is configured to pull the first pulley and the second pulley in opposite rotating directions.

According to one embodiment, the first flexible wire configured to engage with the rim of the first pulley is the same as the third flexible wire configured to engage with the rim of the second pulley, and the second flexible wire configured to engage with the rim of the first pulley is the same as the fourth flexible wire configured to engage with the rim of the second pulley. The first rotating direction of the first pulley is opposite to the first rotating direction of the second pulley, and the second rotating direction of the first pulley is opposite to the second rotating direction of the second pulley.

According to one embodiment, the local portion of the operation device further comprises a second local motor coupled to the receiving portion of the second conversion device, and the second local motor is configured to generate a movement having a second input local force and a second input local speed. The second local motor may be a piezoelectric motor. The second conversion device may be a gear system configured to reduce the second input local speed and increase the second input local force.

According to one embodiment, the remote portion of the operation device further comprises a third remote motor coupled to the receiving portion of the second conversion device and configured to generate a movement having a third input remote speed and a third input remote force. The third remote motor may be a piezoelectric motor. The remote portion of the operation device may further comprise a third remote gear system configured to reduce the third input remote speed and increase the third input remote force.

According to one embodiment, the remote portion of the operation device further comprises a fourth remote motor coupled to the receiving portion of the second conversion device and configured to generate a movement having a fourth input remote speed and a fourth input remote force.

The fourth remote motor may be a piezoelectric motor. The remote portion of the operation device may further comprise a fourth remote gear system configured to reduce the fourth input remote speed and increase the fourth input remote force.

According to one embodiment, the operation device further comprises a third flexible wire configured to transmit the third input remote force from the third remote motor to the receiving portion of the second conversion device.

According to one embodiment, the operation device further comprises a fourth flexible wire configured to transmit the fourth input remote force from the fourth remote motor to the receiving portion of the second conversion device, wherein the third flexible wire is configured to induce a rotation of the third member rotating section in a first rotating direction of the third member rotating section, and the fourth flexible wire is configured to induce a rotation of the third member rotating section in a second rotating direction of the third member rotating section opposite to the first rotating direction of the third member rotating section.

According to one embodiment, the first flexible wire is the same as the third flexible wire, the first remote motor is the same as the third remote motor, and the first flexible wire is configured to rotate the first member rotating section and third member rotating section in opposite rotating directions.

According to one embodiment, the first flexible wire is the same as the third flexible wire, the first remote motor is the same as the third remote motor, the second flexible wire is the same as the fourth flexible wire and the second remote motor is the same as the fourth remote motor. The first rotating direction of the first member rotating section is opposite to the first rotating direction of the third member rotating section, and the second rotating direction of the first member rotating section is opposite to the second rotating direction of the third member rotating section.

According to one embodiment, the operation device further comprises a third hydraulic system configured to transmit the third input remote force from the third remote motor to the receiving portion of the second conversion device. The third hydraulic system may comprises a third hydraulic conduit, a third remote hydraulic cylinder and a third local hydraulic cylinder. The third remote hydraulic cylinder is placed in the remote portion of the operation device, and is coupled to the third remote motor and to the third hydraulic conduit. The third local hydraulic cylinder is placed in the local portion of the operation device, and is coupled to the third hydraulic conduit and to the receiving portion of the second conversion device.

According to one embodiment, the operation device further comprises a fourth hydraulic system configured to transmit the fourth input remote force from the fourth remote motor to the receiving portion of the second conversion device. The third hydraulic system is configured to induce a rotation of the third member rotating section in a first rotating direction of the third member rotating section, and the fourth hydraulic system is configured to induce a rotation of the third member rotating section in a second rotating direction of the third member rotating section opposite to the first rotating direction of the third member rotating section. The fourth hydraulic system may comprise a fourth hydraulic conduit, a fourth remote hydraulic cylinder and a fourth local hydraulic cylinder. The fourth remote hydraulic cylinder is placed in the remote portion of the operation device, and is coupled to the fourth remote motor and to the fourth hydraulic conduit. The fourth local hydraulic cylinder is placed in the local portion of the operation device, and is coupled to the fourth hydraulic conduit and to the receiving portion of the second conversion device.

According to one embodiment, the first hydraulic system is the same as the third hydraulic system, the first remote motor is the same as the third remote motor, and the first hydraulic system is configured to rotate the first member rotating section and third member rotating section in opposite rotating directions.

According to one embodiment, the first hydraulic system is the same as the third hydraulic system, the first remote motor is the same as the third remote motor, the second hydraulic system is the same as the fourth hydraulic system and the second remote motor is the same as the fourth remote motor.

The first rotating direction of the first member rotating section is opposite to the first rotating direction of the third member rotating section, and the second rotating direction of the first member rotating section is opposite to the second rotating direction of the third member rotating section.

A medical device for actively stretching a stomach wall of a patient for creating a sensation of satiety is further provided. The medical device being implantable and comprised an operable first member configured to be fixated to a first portion of the stomach wall, a second member configured to be fixated to a second portion of the stomach wall, and an operation device configured to move at least a portion of the first member in a first direction relative to at least one portion of the second member, for stretching a portion of the stomach wall between the first and second members. The operation device is further adapted to at least one of: move at least a portion of the operable first member in a second direction relative to at least one portion of the second member, and move an operable third member in a third direction for stretching a portion of the stomach wall between the third and second members. The medical device further comprises a mechanical switching device configured to switch the operation device from operating the first member to move in the first direction to operating the first member to move in the second direction, or from operating the first member to move in the first direction to operating the third member to move in the third direction.

According to one embodiment, the operation device comprises a propulsion device, a first operating element, and a second operating element. The mechanical switching device is configured to switch the operation device by altering a position of the propulsion device, such that the propulsion device in a first position operates the first operating element and in a second position operates the second operating element.

According to one embodiment, at least one of the first and second operating element is configured to, directly or indirectly, pull at least one portion of the operable first member in a direction towards at least one portion of the second member.

The medical device may further comprise an operable third member configured to be fixated to a third portion of the stomach wall. The second operating element may be configured to, directly or indirectly, pull at least one portion of the operable third member in a direction towards at least one portion of the second member.

According to one embodiment, the medical device further comprises at least one flexible element connected to the first operating element and, directly or indirectly, connected to the operable first member for pulling the operable first member in the first direction towards at least one portion of the second member.

According to one embodiment, the medical device further comprises at least one flexible element connected to the second operating element and, directly or indirectly, connected to at least one of the operable first member and operable third member, for pulling the operable first member in the second direction towards at least one portion of the second member, and/or the operable third member in a direction towards at least one portion of the second member.

According to one embodiment, the at least one flexible element comprises a flexible wire.

The medical device may further comprise at least one pulley, and the at least one flexible element may be configured to engage the pulley such that transmission is created.

According to one embodiment, the pulley is connected to one of the first member, the second member and the third member.

At least one of the first and second operating element may comprise a drum or spool.

According to one embodiment, at least one of the first and second operating element is configured to, directly or indirectly, push at least one portion of the operable first member in a direction away from at least one portion of the second member.

The medical device may further comprise an operable third member configured to be fixated to a third portion of the stomach wall. The second operating element is configured to, directly or indirectly, push at least one portion of the operable third member in a direction away from at least one portion of the second member.

According to one embodiment, the medical device further comprises at least one shaft, rod or Bowden cable connected to the first operating element and, directly or indirectly, connected to the operable first member for pushing the operable first member in the first direction away from at least one portion of the second member.

According to one embodiment, the medical device further comprises at least one shaft, rod or Bowden cable connected to the second operating element and, directly or indirectly, connected to at least one of the operable first member and the operable third member, for pushing the operable first member in the second direction away from at least one portion of the second member, and/or the operable third member in a direction away from at least one portion of the second member.

According to one embodiment, the medical device further comprises at least one sheath configured to house at least one of the flexible element, the pulley, the shaft, the rod or the Bowden cable.

According to one embodiment, the sheath comprises elevated and lowered portions, such that the sheath can remain flexible even as fibrotic tissue overgrows the sheath. E.g., at least a portion of the sheath may be shaped like a bellows.

According to one embodiment, at least one of the first and second operating element is configured to, directly or indirectly, pivot at least one portion of the operable first member relative to at least one portion of the second member.

According to one embodiment, the medical device further comprises an operable third member configured to be fixated to a third portion of the stomach wall, wherein the second operating element is configured to, directly or indirectly, pivot at least one portion of the operable third member relative to at least one portion of the second member.

According to one embodiment, the medical device further comprises at least one screw, gear wheel or worm wheel connected to the first operating element and, directly or indirectly, connected to the operable first member for pivoting the operable first member relative to at least one portion of the second member.

According to one embodiment, the medical device further comprises at least one screw, gear wheel or worm wheel connected to the second operating element and, directly or indirectly, connected to at least one of the operable first member and operable third member, for pivoting the operable first member relative to at least one portion of the second member, and/or the operable third member relative to at least one portion of the second member.

According to one embodiment, the propulsion device comprises a shaft and the shaft comprises an engaging portion. The switch is configured to move the shaft in an axial direction, such that the engaging portion can move from a first position to a second position, such that the engaging portion, in the first position operatively engages the first operating element, and in the second position does not operatively engage the first operating element.

According to one embodiment, the propulsion device comprises a shaft, and the shaft comprises an engaging portion. The switch is configured to move the shaft in a substantially radial direction, such that the engaging portion can move from a first position to a second position. In the first position, the engaging portion operatively engages the first operating element, and in the second position, the engaging portion does not operatively engage the first operating element.

According to one embodiment, the engaging portion in the first position operatively engages the first operating element, and in the second position operatively engages the second operating element.

According to one embodiment, the shaft further comprises a second engaging portion, and the switch is configured to move the shaft in an axial direction, such that the first and second engaging portions can move from a first position to a second position. The first engaging portion, in the first position, operatively engages the first operating element, and the second engaging portion, in the second position, operatively engages the second operating element.

According to one embodiment, the shaft further comprises a second engaging portion, and the switch is configured to move the shaft in a substantially radial direction, such that the first and second engaging portions can move from a first position to a second position. The first engaging portion, in the first position, operatively engages the first operating element, and the second engaging portion, in the second position, operatively engages the second operating element.

The operation device may further comprise a third operating element, and the shaft may further comprise a third engaging portion. The switch may be configured to move the shaft in the axial direction of the shaft, such that the first, second and third engaging portions can move from the first position to the second position and from the second position to a third position. The first engaging portion, in the first position, may operatively engage the first operating element, and the second engaging portion may, in the second position, operatively engages the second operating element, and the third engaging portion may, in the third position operatively engages the third operating element.

According to one embodiment, the shaft comprises at least one flexible portion.

According to one embodiment, the second member comprises at least one recess or channel configured to house the shaft.

According to one embodiment, the angle between the first and second directions exceeds 20°, preferably exceeds 30°, and most preferably exceeds 45°.

According to one embodiment, the first direction is substantially opposite to the second direction.

The switch could in any of the embodiments be an electrically powered switch and may comprise an electrical motor or solenoid.

According to one embodiment, the switch and the operable first member are operated by a single electrical motor.

According to one embodiment, the switch is operable by centrifugal force. The switch may comprise a force output element operable by centrifugal force, such that the element, when rotated at a first speed, engages a first force receiving element, and when rotated at a second speed, engages a second force receiving element.

According to one embodiment, the medical device further comprises a shaft, and the first force receiving element is connected to the shaft such that rotation of the first force receiving element causes rotation in the shaft, and the second force receiving element comprises a transmission configured to transform a rotating force into a linear force, such that rotation of the first force receiving element causes linear movement of the rotation in the shaft.

According to one embodiment, the linear movement causes the switch in the operation device from operating the first member to move in the first direction to operating the first member to move in the second direction, or from operating the first member to move in the first direction to operating the third member to move in the third direction.

According to one embodiment, the medical device comprises an electrical motor and a speed controller configured to control the speed of the electrical motor.

According to one embodiment, the speed controller is configured to control the speed of the electrical motor, such that the electrical motor, at a first speed, engages the first force receiving element, at a second speed engages the second force receiving element.

According to one embodiment, the operation device is configured to be placed at least partially in a remote unit. The operation device may comprise an electrical motor placed in the remote unit.

According to one embodiment, the switch is configured to be placed at least partially in a remote unit.

According to one embodiment, the operation device is configured to move the first or third member relative to the second member by displacing the first or third member relative to the second member.

According to one embodiment, the first member is elongated and comprises a first main length axis in the direction of the elongation, and the second member is elongated and comprises a second main length axis in the direction of the elongation. The operation device may be configured to displace the first member relative to the second member, such that an angle between the first main length axis and the second main length axis is altered.

According to one embodiment, the operation device is configured to move the first or third member relative to the second member by deforming the first or third member.

According to one embodiment, the operation device is connected to second member such that the connection with the second member provides counterforce against the force needed to move at least a portion of the first or third member relative to the second member.

The first member may comprise at least one curvature and at least a portion of the first member may form a circular arc.

According to one embodiment, a portion of the first member is configured to deform, and wherein the portion of the first member configured to deform comprises at least one of the curvature and the circular arc.

The second member may comprise at least one curvature, and at least a portion of the second member may form a circular arc.

According to one embodiment, the circular are of the first member has a first radius, and the circular are of the second member has a second radius, and the second radius is larger than the first radius.

According to one embodiment, at least one portion of the second member is fixedly fixated to the first member, and the second member may comprise a connecting portion for connecting the second member to the first member.

The footprint of the first member may be configured to enclose a portion of the stomach, and the footprint of the second member may be configured to enclose a portion of the stomach wall.

According to one embodiment, the footprint of the first member is smaller than the footprint of the second member.

According to one embodiment, the second member is configured to encircle a portion of the stomach wall of the patient, and may therefor comprise a simple closed curve. According to one embodiment, the second member is ring shaped.

According to one embodiment, the first member is configured to encircle a portion of the stomach wall of the patient, and the first member may comprise a simple closed curve. E.g., the first member may be ring-shaped.

According to one embodiment, the first member comprises at least one flexible portion, and the first member is configured to deform by deformation of the at least one flexible portion.

The first flexible portion may comprise at least one elastic portion, which may comprise at least one elastic material.

According to one embodiment, the first member comprises at least one articulating joint, and the first member may be configured to deform by articulation of the at least one articulating joint.

The first and/or second members may be configured to be at least partially invaginated by the stomach wall.

According to one embodiment, the medical device is configured to be completely invaginated by the stomach wall, apart from elements for transferring force or communication to or from the medical device.

According to one embodiment, the first and second members are parts of a main portion placed in proximity to the stomach wall, and a portion of the operation device is placed in the main portion. The portion of the operation device placed in the main portion may comprise a receiving portion configured to receive mechanical force from the remote unit.

According to one embodiment, the main portion may comprise a transmission for transforming the received mechanical force into a force for operating the first member for stretching the stomach wall.

According to one embodiment, the transmission comprises a gear or pulley system configured to reduce the speed and increase the force of the received mechanical force. A first flexible element may be configured to travel from a first point on the second member to a pulley such that the pulley is moved closer to the first point by the first flexible element being pulled in a direction towards the first point, and the transmission created by the pulley causes the pulley to move towards the first point at a lower speed than the first flexible element moves towards the first point.

According to one embodiment, at least one of the first operating element and the second operating element is configured to receive a rotating mechanical force, and a transmission is configured to transform the received rotating mechanical force into a liner mechanical force.

The second member may be substantially rigid and a major portion of the second member may be made from a material having a modulus of elasticity in the range 50 GPa-400 GPa.

According to one embodiment, at least one of the length and the cross-sectional area of at least one of the first and second member may be adjustable for calibrating the stretching of the stomach wall.

According to one embodiment, the medical device further comprises a remote unit configured to house at least a portion of the operation device.

According to one embodiment, the remote unit may further comprise an energy storage unit for directly or indirectly energizing the medical device, and/or a controller for controlling the operation device. The controller may comprise a wireless transceiver for communicating wirelessly with a source external to the body of the patient.

According to one embodiment, the medical device further comprises at least one sensor configured to sense a physical parameter of the medical device system.

According to one embodiment, the sensor is a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, and a hydraulic pressure in at least one of the hydraulic actuator, the hydraulic pump, the hydraulic reservoir and the hydraulic conduit.

A method of implanting a medical device for actively stretching a stomach wall of a patient for creating a sensation of satiety is further provided. The medical device comprises a second member configured to be fixated to the stomach wall of the patient and comprising a closed curve, and a first member configured to be fixated to the stomach wall of the patient within the footprint of the second member. According to one embodiment the first member is operably connected to the second member such that the first member can be displaced relative to the second member, and within the footprint of the second member, for stretching a portion of the stomach wall of the patient between the second and first member. The method comprises making an incision in the abdomen of the patient, for accessing the stomach, dissecting a portion of the stomach, inserting the medical device into the abdomen of the patient, fixating the second member to the stomach wall, and fixating the first member to the stomach wall, within the footprint of the second member.

A method of implanting a medical device for actively stretching a stomach wall of a patient for creating a sensation of satiety is further provided. The medical device comprises a second member configured to be fixated to the stomach wall of the patient, and an operable first member connected to the second member and configured to be fixated to the stomach wall at least partially within the footprint of the second member. The first member is configured to be operated to deform such that a first portion of the first member is moved away from a first portion of the second member, for stretching a portion of the stomach wall between the first portion of the first member and the first portion of the second member. The method comprises making an incision in the abdomen of the patient, for accessing the stomach, dissecting a portion of the stomach, inserting the medical device into the abdomen of the patient, fixating the second member to the stomach wall, and fixating the first member to the stomach wall, at least partially within the footprint of the second member.

A method of implanting a medical device for actively stretching a stomach wall of a patient for creating a sensation of satiety is further provided. The medical device comprises an operable first member configured to be fixated to a first portion of the stomach wall, and a second member configured to be fixated to a second portion of the stomach wall, and an operation device. The operation device is configured to pull at least a portion of the first member in a first direction away from at least one portion of the second member, for stretching a portion of the stomach wall between the first portion of the stomach wall and the second portion of the stomach wall for creating the sensation of satiety. The method comprises making an incision in the abdomen of the patient, for accessing the stomach, dissecting a portion of the stomach, inserting the medical device into the abdomen of the patient, fixating the operable first member to a first portion of the stomach wall, and fixating the second member to a second portion of the stomach wall.

A method of implanting a medical device for actively stretching a stomach wall of a patient for creating a sensation of satiety is further provided. The medical device comprises an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety. A portion of the operation device is placed in a remote unit configured to be placed at a remote location in the body of the patient, and a force transferring element configured to transfer mechanical force from the remote unit to the first member, for operating the first member. The method comprising making an incision in the abdomen of the patient, for accessing the stomach, dissecting a portion of the stomach, inserting the medical device into the abdomen of the patient, fixating the operable first member to a first portion of the stomach wall, and fixating the second member to a second portion of the stomach wall.

A method of implanting a medical device for actively stretching a stomach wall of a patient for creating a sensation of satiety is further provided. The medical device comprises an operable first member configured to be fixated to a first portion of the stomach wall, a second member configured to be fixated to a second portion of the stomach wall and an operation device configured to move at least a portion of the first member in a first direction relative to at least one portion of the second member, for stretching a portion of the stomach wall between the first and second members. The operation device is further configured to move at least a portion of the operable first member in a second direction relative to at least one portion of the second member, and move an operable third member in a third direction for stretching a portion of the stomach wall between the third and second members. The medical device further comprising a mechanical switching device configured to switch the operation device from operating the first member to move in the first direction to operating the first member to move in the second direction, or from operating the first member to move in the first direction to operating the third member to move in the third direction. The method comprised making an incision in the abdomen of the patient, for accessing the stomach, dissecting a portion of the stomach, inserting the medical device into the abdomen of the patient, fixating the operable first member to a first portion of the stomach wall, and fixating the second member to a second portion of the stomach wall.

At least one of the steps of fixating the first member to a first portion of the stomach wall and fixating the second member to a second portion of the stomach wall may in any of the methods comprise at least partially invaginated at least one of the first and second members by the tissue of the stomach wall.

At least one of the steps of fixating the first member to a first portion of the stomach wall and fixating the second member to a second portion of the stomach wall may in any of the methods comprise fixating at least one of the first and second member to tissue of the stomach wall using at least one of sutures, staplers or tissue growth promoting structure.

Any one of the methods may further comprise the step of placing at least one sensor in the area of the esophagus of the patient for sensing a parameter related to the patient swallowing. The step of placing at least one sensor in the area of the esophagus of the patient for sensing a parameter related to the patient swallowing may comprise placing at least one of: a motility sensor, an acoustic sensor, an optical sensor or a strain sensor.

Any one of the methods may further comprise the step of placing at least one sensor in the area of the stomach of the patient for sensing a parameter related to the patient eating. The step of placing at least one sensor in the area of the stomach of the patient for sensing a parameter related to the patient eating may comprise placing at least one of temperature sensor, blood saturation or oxygenation sensor, a blood pressure sensor, a sensor configured to sense a parameter related to an ischemia marker, or a pH sensor. The step of placing a pH sensor may comprises sensing the acidity in the stomach.

Any one of the methods may further comprise the step of placing an electrode arrangement configured to be arranged between the medical device and the stomach to engage and electrically stimulate muscle tissue of the stomach to exercise the muscle tissue to improve the conditions for long term implantation of the medical device.

Any one of the methods may further comprise the step of placing an implantable remote unit connected to the medical device in the body of the patient. The step of placing an implantable remote unit connected to the medical device in the body of the patient may comprise placing an implantable remote unit comprising at least a portion of the operation device. The step of placing an implantable remote unit may comprise placing an implantable remote unit comprising an energy storage unit. The step of placing an implantable remote unit connected to the medical device in the body of the patient may comprise placing an implantable remote unit comprising a wireless energy receiver. The step of placing an implantable remote unit connected to the medical device in the body of the patient may comprise placing an implantable remote unit comprising a wireless transceiver for wireless communication.

An implantable operation device for operating a body engaging portion of a medical implant is further provided. The implantable operation device comprises an electrical machine for transforming electrical energy to mechanical force, a transmission for transforming rotational force created by the electrical machine to a linear force and a shaft for transferring rotational and linear force from the operation device to the body engaging portion of the medical implant. The implantable operation device further comprises a switch placed between the electrical machine and the shaft. The switch is configured to switch the force exerted on shaft such that: in a first state, a rotational force is exerted on the shaft, and in a second, a linear force is exerted in the shaft. By means of the switch, several elements may be operated by the same electrical motor.

According to one embodiment, the shaft is configured to transfer a rotational force when the switch is in the first state and transfer an axial force when the switch is in the second state.

According to one embodiment, the shaft comprises an engaging portion, and the electrical machine is configured to move the shaft in an axial direction, such that the engaging portion can move from a first position to a second position. The engaging portion operatively engages a first operating element in the first position, and does not operatively engage the first operating element in the second position.

According to one embodiment, the shaft comprises an engaging portion, and the electrical machine is configured to move the shaft in an axial direction, such that the engaging portion can move from a first position to a second position. In the first position, the engaging portion operatively engages a first operating element, and in the second position, the engaging portion operatively engages a second operating element.

According to one embodiment, the shaft comprises at least one flexible portion. The shaft may be a flexible wire, such as a metal or polymer wire, such as a Bowden cable.

According to one embodiment, the implantable operation device further comprises at least one sheath configured to house at least a portion of the shaft. The sheath may be flexible sheath and may be configured to travel from the operation device to the body engaging portion of the medical implant.

The sheath may comprise elevated and lowered portions, such that the sheath can remain flexible even as fibrotic tissue overgrows the sheath.

According to one embodiment, the switch is an electrically powered switch. The switch may be operable by the electrical machine which may be an electrical motor. Alternatively, the implantable operation device further comprises an additional electrical motor or a solenoid configured to operate the switch.

According to one embodiment, the switch is operable by centrifugal force. The switch may comprise a force output element operable by centrifugal force, such that the force output element, when rotated at a first speed engages a first force receiving element, and when rotated at a second speed engages a second force receiving element.

The first force receiving element may be connected to the shaft such that rotation of the first force receiving element causes rotation in the shaft, and the second force receiving element may comprise the transmission configured to transform the rotating force into a linear force, such that rotation of the first force receiving element causes linear movement of the shaft.

The implantable operation device may further comprise a speed controller configured to control the speed of the electrical motor. The speed controller may be configured to control the speed of the electrical motor, such that the electrical motor, at a first speed, engages the first force receiving element, at a second speed engages the second force receiving element.

According to one embodiment, the implantable operation device is configured to be placed at least partially in a remote unit.

The implantable operation device may further comprise a pre-tensioning device for creating a pre-tension in the shaft. The pre-tensioning device may comprise a flexible element which may be a spring and/or an elastic material.

According to one embodiment, the implantable operation device further comprises a gear system for transforming the mechanical force created by the electrical machine from a force having a first speed and a first strength to a force having a second lower speed and a second higher strength.

An implantable system comprising the implantable operation device according to any one of the preceding embodiments and a body engaging portion configured to exert a force on a body portion of the patient is further provided.

According to an embodiment of the inventive concept, any of devices for actively stretching a stomach wall of a patient for creating a sensation of satiety shown herein may be connected to an implantable energized medical device configured to be held in position by a tissue portion of a patient. The medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, a connecting portion configured to be placed through a hole in the tissue portion extending between the first and second sides of the tissue portion, the connecting portion having a third cross-sectional area in a third plane and being configured to connect the first portion to the second portion, and a hermetic seal arrangement configured to enclose the connecting portion so as to prevent fluid from the patient to enter the connecting portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, and the connecting portion comprises a flexible structure enabling the connecting portion to flex.

In some embodiments, the flexible structure is configured to allow the connecting portion to flex in more than one direction.

In some embodiments, the flexible structure is configured to allow the connecting portion to flex in all directions.

In some embodiments, the flexible structure comprises a bellows.

In some embodiments, the bellows is a metallic bellows.

In some embodiments, the metallic bellows is welded.

In some embodiments, the bellows is a titanium bellows.

In some embodiments, the bellows form part of the hermetic seal arrangement.

In some embodiments, the flexible structure comprises elevated and lowered portions enabling said flexing of the connecting portion.

In some embodiments, the elevated and lowered portions are configured to enable the connecting portion to be compressed and/or expanded.

In some embodiments, the flexible structure has a substantially cylindrical shape.

In some embodiments, the flexible structure is configured to seal against the first portion and/or the second portion.

In some embodiments, the connecting portion and the second portion are hermetically sealed from the first portion.

In some embodiments, the hermetic seal arrangement encloses the connecting portion and the second portion so as to hermetically seal the connecting portion and the second portion from the first portion.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device,

the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical device further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, any of the devices for actively stretching a stomach wall of a patient for creating a sensation of satiety shown herein may be connected to an implantable energized medical device configured to be held in position by a tissue portion of a patient. The medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, wherein the connecting portion and the second portion are configured to form a unit having a central axis extending from a first end of said unit to a second end of said unit, the first end being proximal to the first portion and the second end being distal to the first portion, wherein a physical footprint of said unit perpendicular to the central axis decreases continuously or stepwise from the first end to the second end of said unit.

In some embodiments, said physical footprint comprises a cross-sectional area perpendicular to the central axis.

In some embodiments, the connecting portion and the second portion are one of: configured to reversibly connect to each other to form said unit; or configured to irreversibly connect to each other to form said unit; or configured as a single body forming said unit.

In some embodiments, said unit comprises an angled section forming a bend in said unit.

In some embodiments, the bend is between 150 and 165°, such as between 30° and 150°, such as between 450 and 135°, such as substantially 90°.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical device further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, any of the devices for actively stretching a stomach wall of a patient for creating a sensation of satiety shown herein may be connected to an implantable energized medical remote unit configured to be held in position by a tissue portion of a patient. The medical remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, wherein the connecting portion is configured to extend between the first portion and the second portion along a central extension axis, and wherein the second portion is configured to extend in a length direction being divergent with the central extension axis, and wherein the connecting portion has a decreasing cross-sectional area in a direction from the first portion towards the second portion along the central extension axis, wherein a largest cross-sectional area of the second portion in the length direction is smaller than a smallest cross-sectional area of the connecting portion in said direction from the first portion towards the second portion along the central extension axis, and wherein the second portion further has a decreasing cross-sectional area in the length direction from a first end of the second portion proximal to the connecting portion to a second end of the second portion distal to the connecting portion.

According to an embodiment of the inventive concept, any of the devices for actively stretching a stomach wall of a patient for creating a sensation of satiety shown herein may be connected to an implantable energized medical remote unit configured to be held in position by a tissue portion of a patient. The medical remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, a connecting portion configured to be placed through a hole in the tissue portion extending between the first and second sides of the tissue portion, the connecting portion having a third cross-sectional area in a third plane and being configured to connect the first portion to the second portion, and an electric motor, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, at least part of the electric motor is arranged within the connecting portion.

In some embodiments, the electric motor is arranged within the connecting portion within an imaginary boundary defined by the first surface of the first portion extending through the connecting portion.

In some embodiments, the electric motor is arranged within the connecting portion within an imaginary boundary defined by the second surface of the second portion extending through the connecting portion.

In some embodiments, the electric motor is fully arranged in the connecting portion within imaginary boundaries defined by the first surface of the first portion extending through the connecting portion and the second surface of the second portion extending through the connecting portion respectively.

In some embodiments, the electric motor is arranged such that its longest dimension extends in a direction substantially perpendicular to the first, second and third cross-sectional areas.

In some embodiments, the electric motor is arranged such that its longest dimension extends in a direction between the first portion and the second portion.

In some embodiments, the worm drive is configured to transfer mechanical force from the electric motor to an implantable body engaging portion being external to the implantable energized medical remote unit.

In some embodiments, the electric motor extends through the connecting portion into the first portion and/or the second portion.

In some embodiments, the electric motor extends through an imaginary boundary defined by the first surface of the first portion extending through the connecting portion.

In some embodiments, the electric motor extends through an imaginary boundary defined by the second surface of the second portion extending through the connecting portion.

In some embodiments, the electric motor extends through imaginary boundaries defined by the first surface of the first portion extending through the connecting portion and the second surface of the second portion extending through the connecting portion respectively.

In some embodiments, the implantable energized medical remote unit further comprises a gear arrangement operatively connected to the electric motor wherein the gear arrangement is partly or fully arranged in one of the first portion and the second portion.

In some embodiments, the gear arrangement is arranged within the connecting portion within an imaginary boundary defined by the first surface of the first portion extending through the connecting portion.

In some embodiments, the gear arrangement is arranged within the connecting portion within an imaginary boundary defined by the second surface of the second portion extending through the connecting portion.

In some embodiments, the gear arrangement is fully arranged in the connecting portion within imaginary boundaries defined by the first surface of the first portion extending through the connecting portion and the second surface of the second portion extending through the connecting portion respectively.

In some embodiments, the gear arrangement extends through the connecting portion into the first portion and/or the second portion.

In some embodiments, the gear arrangement extends through an imaginary boundary defined by the first surface of the first portion extending through the connecting portion.

In some embodiments, the gear arrangement extends through an imaginary boundary defined by the second surface of the second portion extending through the connecting portion.

In some embodiments, the gear arrangement extends through imaginary boundaries defined by the first surface of the first portion extending through the connecting portion and the second surface of the second portion extending through the connecting portion respectively.

In some embodiments, the gear arrangement is configured to transfer mechanical force from the electric motor to an implantable body engaging portion being external to the implantable energized medical remote unit.

In some embodiments, the gear arrangement is a worm drive or comprises a worm drive.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical remote unit further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical remote unit further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, any of the devices for actively stretching a stomach wall of a patient for creating a sensation of satiety shown herein may be connected to an implantable energized medical remote unit configured to be held in position by a tissue portion of a patient. The medical remote unit comprising: a first portion configured to be placed on a first side of the tissue portion, the first portion comprising a first surface configured to face a first tissue surface of the first side of the tissue portion, the first portion being further configured to connect, directly or indirectly, to a second portion placed on a second side of the tissue portion opposing the first side, wherein the first portion comprises an internal wireless energy transmitter configured to transmit energy wirelessly to the second portion.

In some embodiments, the first portion is configured to connect, directly or indirectly, to the second portion, via a connecting portion configured to extend through a hole in the tissue portion, the hole extending between the first side of the tissue portion and the second side of the tissue portion.

In some embodiments, the implantable energized medical remote unit further comprises the connecting portion.

In some embodiments, the connecting portion is integrally formed with the first portion.

In some embodiments, the connecting portion is a separate component with regard to the first portion, the connecting portion being configured to be connected to the first portion.

In some embodiments, the first portion has a first cross-sectional area in a first plane and the connecting portion has a second cross-sectional area in a second plane, wherein the first and second planes are parallel to each other, wherein the second cross-sectional area is smaller than the first cross-sectional area, such that the first portion and the second portion are prevented from travelling through the hole in the tissue portion in a direction perpendicular to the first and second planes.

In some embodiments, the first portion is configured to detachably connect, directly or indirectly, to the second portion.

In some embodiments, the first portion comprises a first wireless energy receiver for receiving energy transmitted wirelessly by an external wireless energy transmitter.

In some embodiments, the first portion comprises a first energy storage unit connected to the first wireless energy receiver.

In some embodiments, the first energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to a second wireless energy receiver in the second portion.

In some embodiments, the first portion comprises a first controller comprising at least one processing unit.

In some embodiments, the first controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the connecting portion comprises a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the first portion is excentric with respect to the first portion.

In some embodiments, the implantable energized medical remote unit further comprises a first wireless communication receiver configured to receive communication signals from outside the patient's body.

In some embodiments, the implantable energized medical remote unit further comprises a second wireless communication transmitter arranged in the second portion, wherein the second wireless communication transmitter is configured to transmit communication signals to the first wireless communication receiver.

In some embodiments, the implantable energized medical remote unit further comprises a first wireless communication transmitter arranged in the first portion, the first wireless communication transmitter being configured to transmit communication signals outside of the patient's body.

In some embodiments, the implantable energized medical remote unit further comprises a second wireless communication receiver arranged in the second portion, wherein the first wireless communication transmitter is configured to transmit communication signals to the second wireless communication receiver.

In some embodiments, the implantable energized medical remote unit further comprises a wireless energy receiver configured to receive energy transmitted wirelessly from outside the patient's body and deliver the received energy to the implantable energy storage unit.

In some embodiments, the implantable energized medical remote unit further comprises a control unit configured to control at least one of the body engaging implant, the implantable energy storage unit, the implantable pump, and the implantable electric motor.

In some embodiments, the implantable electric motor is operatively connected to the implantable pump via a rotatable shaft.

In some embodiments, the implantable electric motor is operatively connected to the implantable pump via a magnetic coupling.

In some embodiments, the system further comprises a gear arrangement arranged in the implantable energized medical remote unit and operatively connected to the electric motor, the gear arrangement being configured to reduce the velocity and increase the force of movement generated by the electric motor.

In some embodiments, the system further comprises a gear arrangement arranged externally to the implantable energized medical remote unit and operatively connected to the electric motor, the gear arrangement being configured to reduce the velocity and increase the force of movement generated by the electric motor.

In some embodiments, the system further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the system further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the system further comprises a gear arrangement, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the pump is an hydraulic pump.

In some embodiments, the hydraulic pump comprises a pump comprising at least one compressible hydraulic reservoir.

According to an embodiment of the inventive concept, any of the devices for actively stretching a stomach wall of a patient for creating a sensation of satiety shown herein may be connected to an implantable energized medical remote unit configured to be held in position by a tissue portion of a patient. The medical remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the first portion is configured to be placed subcutaneously in the patient, and wherein the first portion comprises a connecting interface arrangement for transferring wired energy and/or wired communication signals and/or fluid to an additional implant in the patient.

In some embodiments, a height of the first portion measured in a plane perpendicular to the first plane is 15 mm or less, such as 10 mm or less, such as 7 mm or less, such as 5 mm or less.

In some embodiments, the connecting interface arrangement comprises a port for transferring fluid from the first portion to said additional implant.

In some embodiments, the implantable energized medical remote unit further comprises at least one conduit or tube for transferring said fluid, wherein the at least one conduit or tube is connected to the port.

In some embodiments, the implantable energized medical remote unit further comprises at least one wire for energy and/or communication signals connected to the connecting interface arrangement.

In some embodiments, the height of the first portion is a maximum height.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical remote unit further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical remote unit further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, any of the devices for actively stretching a stomach wall of a patient for creating a sensation of satiety shown herein may be connected to an implantable energized medical remote unit configured to be held in position by a tissue portion of a patient. The medical remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the first portion and the second portion are configured to be placed subcutaneously in the patient, such that the implantable energized medical remote unit can be placed with either of the first portion and the second portion on the first side of the tissue portion.

In some embodiments, a height of the second portion measured in a plane perpendicular to the second plane is 15 mm or less, such as 10 mm or less, such as 7 mm or less, such as 5 mm or less.

In some embodiments, the first portion has a length in a plane parallel to the first plane, wherein the second portion has a length in a plane parallel to the second plane, and wherein the length of the first portion differ no more than 30% with regard to the length of the second portion, such as wherein the length of the first portion differ no more than 15% with regard to the length of the second portion, such as wherein the length of the first portion differ no more than 5% with regard to the length of the second portion, such as wherein the length of the first portion differ no more than 1% with regard to the length of the second portion.

In some embodiments, the first portion has a width in a plane parallel to the first plane, wherein the second portion has a width in a plane parallel to the second plane, and wherein the width of the first portion differ no more than 30% with regard to the width of the second portion, such as wherein the width of the first portion differ no more than 15% with regard to the width of the second portion, such as wherein the width of the first portion differ no more than 5% with regard to the width of the second portion, such as wherein the width of the first portion differ no more than 1% with regard to the width of the second portion.

In some embodiments, the first portion has a height in a plane perpendicular to the first plane, and wherein the height of the first portion differ no more than 30% with regard to the height of the second portion, such as wherein the height of the first portion differ no more than 15% with regard to the height of the second portion, such as wherein the height of the first portion differ no more than 5% with regard to the height of the second portion, such as wherein the height of the first portion differ no more than 1% with regard to the height of the second portion.

In some embodiments, a height of the first portion measured in a plane perpendicular to the first plane is 15 mm or less, such as 10 mm or less, such as 7 mm or less, such as 5 mm or less.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical remote unit further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical remote unit further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical remote unit further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, any of the devices for actively stretching a stomach wall of a patient for creating a sensation of satiety shown herein may be connected to an implantable energized medical remote unit configured to be held in position by a tissue portion of a patient. The medical remote unit comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein the second portion comprises or forms a reservoir for holding a fluid; the implantable energized medical remote unit further comprising: a sealed container configured to protrude into the reservoir; an actuator connected to the sealed container, the actuator being configured to expand or retract the sealed container to change the volume of the sealed container for pumping fluid to or from the reservoir; wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes.

In some embodiments, the actuator comprises an electric motor.

In some embodiments, the actuator is arranged in the connecting portion.

In some embodiments, the actuator is partly or fully arranged inside the sealed container.

In some embodiments, the second portion comprises a port in fluid communication with the reservoir for transferring fluid between the reservoir and an additional implant in the patient.

In some embodiments, the implantable energized medical remote unit further comprises a conduit connected to the port, the conduit being configured to transfer fluid between the reservoir and the additional implant.

In some embodiments, the implantable energized medical remote unit further comprises an injection port for introducing fluid, the injection port being arranged in the first portion.

In some embodiments, the implantable energized medical remote unit further comprises an internal conduit connecting the injection port to the reservoir.

In some embodiments, the sealed container is a bellows.

In some embodiments, the bellows is a metallic bellows.

In some embodiments, at least a portion of the sealed container configured to be in contact with fluid comprises metal.

In some embodiments, the volume of the sealed container can be altered such that the volume of the sealed container is more than 60% of the maximum volume of the reservoir.

In some embodiments, the sealed container comprises at least one flexible portion, and wherein the flexible portion enable at least one of compression and expansion of the sealed container.

In some embodiments, the sealed container comprises at least one elastic portion, and wherein the elastic portion enable at least one of compression and expansion of the sealed container.

In some embodiments, the implantable energized medical remote unit further comprises a first energy storage unit and/or a second energy storage unit for powering the actuator.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first energy storage unit is connected to the first wireless energy receiver.

In some embodiments, the second portion comprises the second energy storage unit, wherein the second energy storage unit is connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, the first controller and/or the second controller is configured to control the actuator.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical remote unit further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical remote unit further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical remote unit further comprises a gear arrangement, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

An external system for providing remote instructions to an implantable medical device is further provided. The external system being configured to provide instructions to be transmitted to the implantable medical device, derive a checksum from the instructions, electronically sign the instructions and the checksum. The external system is further configured to form a data packet from the instructions, the electronic signature and the checksum. The implantable medical device further comprises a wireless transmitter configured to wirelessly send the data packet to the implantable medical device. The external system may further be configured to encrypt the data packet at the external system. The checksum is configured to verify that no changes have been made to the bit stream forming the instructions.

According to one embodiment, the wireless transmitter is part of a wireless transceiver comprised in the external system.

According to one embodiment, the external system comprises a first external device and a second external device, and the first external device is configured to transmit the data packet to the second external device, and the second external device is configured to transmit the data packet wirelessly to the implantable medical device without changing the data packet and/or without full decryption of the data packet.

The external system may be configured to transmit at least one instruction for altering the control program of the implantable medical device, to the implantable medical device, which may include altering at least one parameter for affecting the control of the implantable medical device, which may include updating at least one parameter of the control program to a parameter value comprised in a set of parameter values stored in the implantable medical device.

According to one embodiment, the first external device is configured to send the data packet from the first external device to the second external device using a first network protocol and send the data packet from the second external device to the implantable medical device using a second network protocol.

According to one embodiment, the first external device is configured to send the data packet from the first external device to the second external device using wired communication and send the data packet from the second external device to the implantable medical device using wireless communication.

According to one embodiment, the first external device is configured to wirelessly send the data packet from the first external device to the second external device using a first network protocol, and wirelessly send the data packet from the second external device to the implantable medical device using a second network protocol.

According to one embodiment, the first external device is configured to wirelessly send the data packet from the first external device to the second external device using a first frequency band, and wirelessly send the data packet from the second external device to the implantable medical device using a second frequency band.

According to one embodiment, the first external device is configured to wirelessly send the data packet from the first external device to the second external device using a first wireless technology, and wirelessly send the data packet from the second external device to the implantable medical device using a second wireless technology.

According to one embodiment, the external system is configured to electronically sign the instructions at the external system using a key of the external system. The key may be a non-extractable key.

According to one embodiment, the second external device is configured to perform a proof of possession operation comprising the steps of transmitting, form the first external device to the second external device, a query based on a public key associated with the private of the external system, receiving, at the second external device, a response based on the possession of the private key in the first external device, and verifying that the response based on the possession of the private key matches the query based on a public key.

According to one embodiment, the first external device is configured to form the data packet and electronically sign the instruction using a first private key, and the second external device is configured to: receive the data packet from the first external device, verify that the first external device is a trusted transmitter, in response to the verification, electronically sign the data packet using a second private key, and transmit the data packet from the second external device to the medical implant.

According to one embodiment, the first external device is configured to electronically sign the instructions and encrypt the data packet using a key placed on a key device external to the first external device. The external system may comprise a key device configured to hold at least one private key which is part of a public-private key pair used for asymmetric encryption.

According to one embodiment, the key device comprises a wireless transmitter for wirelessly transmitting the at least one private key or a signal based on the private key, to the first external device. The second external device may be configured to at least one of: electronically sign the instructions and encrypt the data packet using a key placed on a key device external to the second external device.

According to one embodiment, the external system further comprises a second key device configured to hold at least one second private key and the second key device may comprise a wireless transmitter for wirelessly transmitting the at least one private key or a signal based on the private key to the second external device.

According to one embodiment, 5. the external system further comprises a second key device comprising a wireless transmitter for wirelessly transmitting at least one second private key or a signal based on the second private key to the first external device.

According to one embodiment, at least one of the key device and the second key device comprises at least one of: a key card, a wearable device and a handset.

The first and/or second external device may be configured to be unlocked by user credentials provided to the first external device. The user credentials may comprise a username and a password and/or a PIN-code.

According to one embodiment, the first external device is configured to verify the user credentials by comparing the user credentials with user credentials stored in the first external device. The user credentials may be stored in the first external device by the manufacturer of the first external device.

The user credentials may be stored as hardware or software in the first external device.

According to one embodiment, the first external device is configured to verify the user credentials by communicating with a remote server.

The external system may in any of the embodiments herein be configured to function without connection to the Internet and may be configured to communicate with the implantable medical device independently of time.

The first and second private keys may be different in any of the embodiments. However, the first and second private keys may comprise at least one common element. At least one first and second external devices are configured to be unlocked by at least one of the first and second private key.

According to one embodiment, the external system comprises a central server, and the central server is configured to form a data packet from the instructions, the electronic signature and the checksum and further configured to provide the formed data packet to the first external device.

The central server may be accessed by at least one healthcare professional, such that the healthcare professional can provide input to the central server for forming the instructions to be sent to the implantable medical device.

The central server may be accessed by at least one patient, such that the patient can provide input to the central server for verifying at least one of: the authenticity of the healthcare professional and the correctness of the instructions. The healthcare provider and/or the patient can electronically sign the instructions at the central server.

According to one embodiment, the central server is configured to verify the authenticity of the first and second key and electronically sign the instructions using the first and second key. The second key may be a user key, and wherein the external system may be configured to use the second key for at least one of approving that communication is transmitted to the implantable medical device, and approving that a healthcare provider prepares an instruction to the implantable medical device.

According to one embodiment, the approval step can be performed by first or second external device.

According to one embodiment, the first key is required to create an instruction to the implantable medical device and the second key is required to transmit the created instruction to the implantable medical device.

According to one embodiment, at least one of the first and second external device comprises an input button configured to be used for verifying user presence.

According to one embodiment, the input button con be configured to replace at least one of: input of at least one key to at least one of the first and second external device, and input of credentials into at least one of the first and second external device. The input button may be configured to replace the second key.

According to one embodiment, the external system is configured to transmit the data packet to the implantable medical device, and the data packet comprises at least one instruction signed by a first key and a public key including information about which root have created the public key.

According to one embodiment, at least one of the first and second external device may be configured to enable communication with the implantable medical device based on at least one password being provided to at least one of the first and second external device.

According to one embodiment, at least one of the first and second external device is configured to enable communication with the implantable medical device based on two passwords being provided to at least one of the first and second external device.

According to one embodiment, at least one of the first and second external device is configured to enable communication with the implantable medical device based on one patient password and one healthcare provider password being provided to at least one of the first and second external device.

According to one embodiment, at least one of the first and second external devices are configured to perform a verification query operation with at least one of the first and second key device, the verification query operation comprising: transmitting, from the first or second external devices, a query comprising a computational challenge to at least one of the first and second key device, receiving, at the first or second external devices, a response based on the transmitted computational challenge, and verifying, at the first or second external devices, the received response. The verification query operation may be in the form of a proof of possession operation comprising: receiving a public key of at least one of the first and second key devices, the public key being associated with a private key of the first or second key device, transmitting, from at least one of the first and second external devices, a computational challenge to the first or second key device, based on the public key received from the first or second key device, receiving a response from the first or second key device based on the possession of the private key in the first or second key device, and verifying that the response based on the possession of the private key matches the query based on a public key.

An implantable medical device configured to receive remote instructions from an external system is further provided. The implantable medical device comprises a wireless receiver configured to receive wirelessly transmitted data packets from the external system, a computing unit configured to: verify the electronic signature, and use a checksum provided in the data packet to verify the integrity of the instructions. The computing unit may further be configured to decrypt the data packet. The computing unit may be configured to use the checksum to verify that the bit stream making up the instructions is unchanged.

The wireless receiver may be part of a wireless transceiver.

According to one embodiment, the computing unit comprises a memory unit configured to store electronic signatures, and the computing unit may be configured to verify the electronic signature by comparing the electronic signature with the electronic signatures stored in the memory unit.

According to one embodiment, the implantable medical device comprises a control program configured to control at least one function of the implantable medical device, and the computing unit may be configured to alter the control program on the basis of the received instructions.

According to one embodiment, the implantable medical device comprises an internal computing unit configured to run a control program for controlling a function of the implantable medical device. The control program may comprise at least one adjustable parameter affecting the control of the implantable medical device, and the method of providing remote instructions may comprise providing instructions for altering the at least one parameter for affecting the control of the implantable medical device.

According to one embodiment, the implantable medical device comprises a central unit, comprising at least one of a wireless receiver and a wireless transceiver, and a security module connected to the central unit. The implantable medical device may be configured to transfer the data packet from the central unit to the security module, and the security module may be configured to perform at least a portion of at least one of the decryption and the signature verification.

The security module may comprise a set of rules for accepting communication from the central unit, and the security module may be configured to verify compliance with the set of rules.

According to one embodiment, the wireless receiver or wireless transceiver may be configured to be placed in an off-mode, in which no wireless communication can be received by the wireless transceiver, and the set of rules may comprise a rule stipulating that communication from the central unit is only accepted at the security module when the wireless transceiver is placed in the off-mode.

According to one embodiment, the implantable medical device may be configured to decrypt the data packet and/or verify the electronic signature using a private key of the implantable medical device. The private key may be a non-extractable key. The private key may be provided in the implantable medical device by the manufacturer of the implantable medical device and may be stored as hardware or software in the implantable medical device.

According to one embodiment, the implantable medical device is configured to perform a proof of possession operation comprising: transmitting, from the implantable medical device to the external system, a query based on a public key associated with the private key of the external system, receiving, at the implantable medical device, a response based on the possession of the private key in the external system, and verifying that the response based on the possession of the private key matches the query based on a public key.

The implantable medical device may be configured to communicate with the external system independently of time.

According to one embodiment, the implantable medical device is configured to: verify a first electronic signature made using at least one of a first key and a second key, and verifying a second electronic signature made using at least one of a first key and a second key. At least one of the first and second keys may be a private key, and the first and second keys may be different, and the first and second keys may comprise at least one common element.

According to one embodiment, the implantable medical device is configured to verify a first electronic signature to allow communication from the external system to the implantable medical device, and verify a second electronic signature to allow an instruction received in the communication to alter the control program running on the implantable medical device.

According to one embodiment, the first electronic signature is an electronic signature linked to the user of the implantable medical device and the second electronic signature is an electronic signature linked to a healthcare provider.

According to one embodiment, only a portion of the private key is needed to at least one of: decrypt the data packet and verify the electronic signature.

The implantable medical device trusts any external device holding the private key.

According to one embodiment, the implantable medical device is configured to receive the data packet comprising: at least one instruction signed by a private key of the external system, and a public key including information about which root have created the public key.

According to one embodiment, the implantable medical device is configured to accept communication from an external system based on at least one password being provided to the implantable medical device. According to one embodiment, the implantable medical device is configured to accept communication from an external system based on two passwords being provided to the implantable medical device.

According to one embodiment, the implantable medical device is configured to accept communication from an external system based on one patient password and one healthcare provider passwords being provided to the implantable medical device.

A method of providing remote instructions from an external system to an implantable medical device is further provided. The method comprises deriving a checksum, at the external system, from the instructions to be sent to the implantable medical device, electronically signing the instructions and the checksum, at the external system, wherein: the instructions, the checksum and the electronic signature form a data packet, wirelessly sending the data packet to the implantable medical device, verifying the electronic signature, and using the checksum to verify the integrity of the instructions.

According to one embodiment, the method further comprises the steps of encrypting the data packet at the external system using a private key of the external system, and decrypting, at the implantable medical device, the data packet using a private key of the implantable medical device.

According to one embodiment, the step of verifying the electronic signature comprises comparing the electronic signature with electronic signatures stored in the implantable medical device.

According to one embodiment, the step of wirelessly sending the data packet to the implantable medical device comprises sending the data packet from a first external device to a second external device using wired communication and wirelessly sending the data packet from the second external device to the implantable medical device.

According to one embodiment, the step of wirelessly sending the data packet to the implantable medical device comprises sending the data packet from a first external device to a second external device and further wirelessly sending the data packet from the second external device to the implantable medical device. The second external device transmits the data packet without changing the data packet and/or without full decryption.

According to one embodiment, the step of wirelessly sending the data packet to the implantable medical device comprises: wirelessly sending the data packet from a first external device to a second external device using a first network protocol, and wirelessly sending the data packet from the second external device to the implantable medical device using a second network protocol.

According to one embodiment, the step of wirelessly sending the data packet to the implantable medical device comprises: wirelessly sending the data packet from a first external device to a second external device using a first frequency band, and wirelessly sending the data packet from the second external device to the implantable medical device using a second frequency band.

According to one embodiment, the step of wirelessly sending the data packet to the implantable medical device comprises: wirelessly sending the data packet from a first external device to a second external device using a first wireless technology, and wirelessly sending the data packet from the second external device to the implantable medical device using a second wireless technology, wherein the first wireless technology has an effective range being one of: 2 times, 4 times, 8 times 20 times, 50 times or 100 times longer than the first wireless technology.

According to one embodiment, the implantable medical device comprises a central unit, comprising a wireless transceiver, and a security module connected to the central unit. The step of decrypting, at the implantable medical device, the data packet, comprises transferring the data packet from the central unit to the security module, and performing at least a portion of the decryption in the security module.

According to one embodiment, the security module comprises a set of rules for accepting communication from the central unit, and the step of transferring the data packet from the receiving unit of the implant to the security module comprises verifying compliance with the set of rules.

According to one embodiment, the wireless transceiver is configured to be placed in an off-mode, in which no wireless communication can be received by the wireless transceiver, and the set of rules comprises a rule stipulating that communication from the central unit is only accepted at the security module when the wireless transceiver is placed in the off-mode.

According to one embodiment, the step of electronically signing the instructions at the external system comprises electronically signing the instructions at the external system using a private key of the external system.

According to one embodiment, the step of verifying the electronic signature comprises performing a proof of possession operation comprising the steps of: transmitting, form the medical device to the external system, a query based on a public key associated with the private of the external system, receiving, at the medical device, a response based on the possession of the private key in the external system, and verifying that the response based on the possession of the private key matches the query based on a public key.

According to one embodiment, the step of forming the data packet is performed at a first external device, and the step of electronically signing the instructions comprises electronically signing the instruction using a first private key, and wherein the method further comprises: transmitting the data packet from the first external device to a second external device, verifying, at the second external device, that the transmitter is a trusted transmitter, in response to the verification, electronically signing the data packet using a second private key, and transmitting the data packet from the second external device to the medical implant, and verifying, at the medical implant, the electronic signatures generated using the first and second private keys. The method may further comprise using the checksum to verify the integrity of the instructions.

According to one embodiment, the method according to any one of the preceding embodiments is performed without connection to the Internet and/or independently of time.

According to one embodiment, the method further comprises the central server being accessed by at least one healthcare professional, and the healthcare professional providing input to the central server for forming the instructions to be sent to the implantable medical device.

According to one embodiment, the central server is accessed by at least one patient, such that the patient can provide input to the central server for verifying at least one of: the authenticity of the healthcare professional and the correctness of the instructions.

According to one embodiment, the healthcare provider may electronically sign the instructions at the central server and/or the patient may electronically sign the instructions at the central server.

According to one embodiment, the method further comprising the steps of: verifying the authenticity of the first and second key at the central server, and electronically signing the instructions using the first and second key.

According to one embodiment, the second key is a user key, and the method may comprise the steps of using the second key for at least one of: approving that communication is transmitted to the implantable medical device, and approving that a healthcare provider prepares an instruction to the implantable medical device.

According to one embodiment, the approval step can be performed by first or second external device.

According to one embodiment, the first key is required to create an instruction to the implantable medical device and the second key is required to transmit the created instruction to the implantable medical device.

At least one of the first and second external device may comprise an input button, and the method may further comprise the step of pressing the button for verifying user presence. The input button may be placed on the second external device.

According to one embodiment, the method further comprises a verification query operation between at least one of the first and second external devices and at least one of the first and second key devices, the verification query operation comprising: transmitting, from the first or second external devices, a query comprising a computational challenge to at least one of the first and second key device, receiving, at the first or second external devices, a response based on the transmitted computational challenge, and verifying, at the first or second external devices, the received response. The verification query operation may be in the form of a proof of possession operation comprising: receiving a public key of at least one of the first and second key devices, the public key being associated with a private key of the first or second key device, transmitting, from at least one of the first and second external devices, a computational challenge to the first or second key device, based on the public key received from the first or second key device, receiving a response from the first or second key device based on the possession of the private key in the first or second key device, and verifying that the response based on the possession of the private key matches the query based on a public key.

A method of providing remote instructions from an external system to an implantable medical device is further provided. The implantable medical device comprises a list of codes and the external system comprises a list of codes. The method comprising encrypting the instructions at the external system using a code from a position on the list of codes, wirelessly sending the encrypted instructions to the implantable medical device, and decrypting, at the implantable medical device, the instructions using a code from a position on the list of codes.

According to one embodiment, the method further comprises the steps of: wirelessly sending position information from the external device to the implantable medical device, and using the information at the implantable medical device for selecting the code from the list of codes.

According to one embodiment, the step of encrypting, at the external system, the instructions using a code from a position on the list of codes comprises selecting the code on a current position on the list of codes, wherein the method further comprises the step of updating the current position to a new current position after using the code.

According to one embodiment, the step of decrypting, at the implantable medical device, the instructions using a code from a position on the list of codes comprises selecting the code on a current position on the list of codes, wherein the method further comprises the step of updating the current position to a new current position after using the code.

According to one embodiment, the current position comprises a number and wherein the step of updating the current position comprises updating the number to a sequential number.

According to one embodiment, the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without changing the encrypted instructions and/or without full decryption of the instructions.

A communication system for transmission of data to or from an implantable medical device is provided. The communication system comprises an implantable medical implant, a first remote control comprising a first wireless communication unit configured for wireless transmission of data to or from the implantable medical device, the first remote control being operable by a user, and a second remote control comprising a second wireless communication unit configured for wireless transmission of control commands or data to or from the implantable medical device, and a third communication unit for communicating with a patient display device, the second remote control being inoperable by a user.

According to an embodiment, the first remote control comprises an input device for receiving a first user input, and wherein the first remote control is configured to transmit the first user input to the implantable medical device.

According to an embodiment, the second remote control is configured to receive second user input from the patient display device and to transmit the second user input to the implantable medical implant.

According to an embodiment, the data comprises a control command for the medical implant.

According to an embodiment, at least one of the first wireless communication unit and the second wireless communication unit is configured to send or receive data using near-field magnetic induction.

According to an embodiment, at least one of the first wireless communication unit and the second wireless communication unit comprises a transmitter coil for modulating a magnetic field for transmitting the data, and wherein the implantable medical implant comprises a receiving coil and an NFMI receiver connected to the receiving coil to receive the data.

According to an embodiment, the transmitter coil is configured to modulate a magnetic field, and the NFMI receiver is adapted to measure the magnetic field in the receiving coil.

According to an embodiment, at least one of the first wireless communication unit and the second wireless communication unit is configured to wirelessly charge the medical implant using near-field magnetic induction.

According to an embodiment, the medical implant comprises a coil for receiving wireless energy for charging the implant via near-field magnetic induction.

According to an embodiment, the second and third communication units are configured to transmit and/or receive data using different network protocols.

According to an embodiment, wherein the second and third communication units are configured to transmit and/or receive data using different frequency bands.

According to an embodiment, at least one of the first remote control, the second remote control and the implantable medical device comprises a Bluetooth transceiver.

According to an embodiment, at least one of first remote control, the second remote control and the implantable medical device comprises a UWB transceiver.

According to an embodiment, the network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to an embodiment, the second communication unit has a longer effective range than the third communication unit.

According to an embodiment, the second remote control is configured to communicate with a consumer electronics device.

According to an embodiment, the patient display device comprises the consumer electronics device.

According to an embodiment, the first remote control is configured to control functions of the implantable medical device based on user input to the first remote control.

According to an embodiment, a method corresponding to the communication system according to the previous aspect is provided.

According to an aspect, a method for wireless energy transfer from an external energy source located outside the patient to an internal energy receiver located inside the patient, the internal energy receiver being connected to an implantable medical device for supplying received energy thereto, is provided. The method comprises determining an accumulated amount of received energy over a time period, determining a current change in the received energy, determining a control signal reflecting the accumulated received energy and the change in the received energy, and controlling the energy transfer based on the control signal.

According to an embodiment, determining an accumulated amount of received energy is determined by the internal energy receiver.

According to an embodiment, determining a current change is performed by the internal energy receiver.

According to an embodiment, the internal energy receiver comprises a PID regulator for controlling the energy transfer.

According to an embodiment, the PID regulator is implemented in a microcontroller.

According to an embodiment, determining a control signal is performed by the internal energy receiver.

According to an embodiment, the control signal is transmitted to the external energy source, and wherein the external energy source is configured to adjust the transmitted energy base on the control signal.

According to an embodiment, controlling the energy transfer is controlled by the internal energy receiver.

According to an embodiment, controlling the energy transfer is performed by the external energy source.

According to an embodiment, controlling the energy transfer comprises adjusting the energy transfer efficiency.

According to an embodiment, the external device comprises a transmitter coil for modulating a magnetic field for transmitting data or transmitting energy, and wherein the implantable medical device comprises a receiving coil and an NFMI receiver connected to the receiving coil to receive the data or the energy.

According to an embodiment, at least one of the first wireless communication unit and the second wireless communication unit is configured to wirelessly charge the medical device using near-field magnetic induction.

According to an embodiment, the medical device comprises a coil for receiving wireless energy for charging the device via near-field magnetic induction.

According to an embodiment, the method further comprises receiving energy in pulses according to a pulse pattern, and measuring the received pulse pattern.

According to an embodiment, the method further comprises determining that the pulse pattern deviates from a predefined pulse pattern, and controlling the energy transfer based on the determination.

According to an embodiment, the method further comprises measuring a temperature in the implantable medical device or in the body of the patient, and controlling the energy transfer in response to the measured temperature.

According to an embodiment, the implantable medical device comprises at least one coil connected to a variable impedance, the method further comprising controlling the energy transfer by controlling the variable impedance.

According to an embodiment, the implantable medical device comprises at least one coil having a plurality of windings, wherein the plurality of windings each are connected to a respective variable impedance, the method further comprising controlling the energy transfer by controlling the respective variable impedance individually.

According to an aspect, an implantable medical device, a first remote control and/or a second remote control configured to perform the method according to the previous aspect are provided.

A corresponding method is also provided.

A method of teaching a voice-controlled implantable medical device to recognize a voice command is provided. The method comprises inputting a first audio training phrase to the implantable medical device, when the implantable medical device is implanted in the body of the patient and creating a transfer function, the transfer function being based on the first audio training phrase, wherein the transfer function is configured to adjust the amplitude of at least one frequency of audio received at the implantable medical device for enhancing audio received at the implantable medical device to facilitate detection of voice commands. The method further comprises inputting a second audio training phrase to the implantable medical device, the second audio training phrase comprising the voice command. The voice command comprises an instruction for the control of the implantable medical device. The method further comprises using the transfer function for generating an enhanced second audio training phrase in the implantable medical device, and associating the enhanced second audio training phrase with the instruction for the control of the implantable medical device.

In an embodiment, adjusting the amplitude comprises at least one of: filtering, cancelling and amplifying the at least one frequency.

In an embodiment, at least one of the first and second audio training phrase is a spoken audio training phrase.

In an embodiment, the spoken audio training phrase is spoken by the patient the implantable medical deivce is implanted in.

In an embodiment, the first audio training phrase comprises the voice command.

In an embodiment, the first and second audio training phrases is the same voice command.

In an embodiment, the first and second audio training phrases are different.

In an embodiment, creating the transfer function comprises amplifying frequencies muffled by the location of the implantable medical device in the body of the patient.

In an embodiment, creating the transfer function comprises filtering or cancelling noise generated by the body.

In an embodiment, the medical implant is configured to receive voice commands related to an instruction for control of the implantable medical device.

In an embodiment, the voice command relates to at least one of: performing a function of the medical device; using a sensor to measure a parameter relating to a condition of the patient or a condition of the implantable medical device; and sending or receiving data from the implantable medical device.

A system corresponding to the preceding aspect is also provided.

According to an aspect, a system for wirelessly charging an implantable medical implant, when implanted in a body of a patient, is provided. The system comprises an internal energy receiver comprising a secondary coil, the internal energy receiver being connected to the implantable medical implant and an external energy transmitter comprising a primary coil for wirelessly transmitting energy to the internal energy receiver via the secondary coil, wherein a diameter of the primary coil is larger than a diameter of the secondary coil.

According to an embodiment, the system further comprises an internal controller connected to the internal energy receiver, for controlling the amount of energy received by the internal energy receiver.

According to an embodiment, the internal energy receiver further comprises a measurement unit for measuring a parameter related to the implantable medical implant or the body of the patient.

According to an embodiment, the controller is configured to measure the accumulated energy received by the internal energy receiver over a period of time and to measure a current change in energy received, and to control the energy received based on the accumulated energy and the current change.

According to an embodiment, the controlled comprises a Proportional—Integral—Derivative, PID, regulator for controlling the received energy.

According to an embodiment, the internal energy received comprises a variable impedance.

According to an embodiment, the internal energy receiver is configured to control the resonant frequency by controlling the variable impedance.

According to an embodiment, the controller is configured to vary the variable impedance in response to a measured parameter deviating from a predetermined interval or exceeding a threshold value.

According to an embodiment, the parameter relates to the energy received by the coil over a time period.

According to an embodiment, the measurement unit is configured to measure a parameter related to a change in energy received by the coil.

According to an embodiment, the receiving unit is configured to receive transcutaneously transferred energy in pulses according to a pulse pattern, and wherein the measurement unit is configured to measure a parameter related to the pulse pattern.

According to an embodiment, the receiving unit is configured to receive transcutaneously transferred energy in pulses according to a pulse pattern, and wherein the measurement unit is configured to measure a parameter related to the pulse pattern.

According to an embodiment, the controller is configured to control the variable impedance in response to the pulse pattern deviating from a predefined pulse pattern.

According to an embodiment, the variable impedance comprises a resistor and a capacitor, the variable impedance comprises a resistor and an inductor, the variable impedance comprises an inductor and a capacitor, the variable impedance comprises a digitally tuned capacitor, the variable impedance comprises a digital potentiometer, or the variable impedance comprises a variable inductor.

According to an embodiment, the diameter of the primary coil is at least one of more than 0.5 cm, more than 10 cm, more than 15 cm, more than 20 cm, coil is more than 30 cm, or is more than 50 cm.

According to an embodiment, the area of the primary coil is more than 0.5 cm2, more than 2 cm2, more than 10 cm2, more than 100 cm2, more than 300 cm2, more than 500 cm2, or more than 800 cm2.

According to an embodiment, a method corresponding to the system for wirelessly charging an implant according to the previous aspect is provided.

According to an aspect, a system for communication with an implantable medical device, when implanted in a body of a patient, is provided. The system comprises an internal communications unit, connected to or comprised in the implantable medical device, and an external communications unit, wherein the internal communications unit and the external communications unit are configured to communicate using near field magnetic induction, NFMI.

According to an embodiment, the internal communication unit comprises an internal NFMI receiver and an internal coil connected to the internal NFMI receiver, the internal NFMI receiver being configured to measure an induced voltage in the internal coil. The external communications unit comprises an external NFMI transmitter and an external coil connected to the external NFMI transmitter, and the external coil and the external NFMI transmitter are configured to modulate a magnetic field for sending data to the implantable medical device via the internal coil.

According to an embodiment, the external NFMI transmitter further comprises a capacitor for tuning.

According to an embodiment, the internal NFMI receiver comprises a tunable resistor and capacitor tank.

According to an embodiment, the internal communication unit comprises an internal NFMI transmitter and an internal coil connected to the internal NFMI transmitter. The external communications unit comprises an external NFMI receiver and an external coil connected to the external NFMI receiver, the external NFMI receiver being configured to measure an induced voltage in the external coil, and the internal coil and the internal NFMI transmitter are configured to modulate a magnetic field for sending data to the external communications unit via the external coil.

According to an embodiment, the internal NFMI transmitter further comprises a capacitor for tuning the internal coil and the internal NFMI transmitter.

According to an embodiment, the external NFMI receiver comprises a tunable resistor and capacitor tank for tuning the external coil and the external NFMI receiver.

According to an embodiment, the implantable medical device comprises an active portion configured to monitor, treat or perform a function of a body of a patient.

According to an embodiment, the active portion is not a pacemaker, a hearing aid or a neurostimulation implant.

According to an embodiment, the internal communications unit is adapted to be implanted at a tissue depth of at least 8 cm or at least 15 cm.

According to an embodiment, the internal communications unit is adapted to be implanted in an abdomen of a patient.

According to an embodiment, the external communications unit is configured to communicate with another external device.

According to an embodiment, the internal communications unit is configured to encrypt data before transmitting it to the external communications unit.

According to an embodiment, the external communications unit is configured to relay the encrypted data to the another external device without decrypting it.

According to an aspect, an implantable medical device adapted to receive wirelessly transmitted energy is provided, the implantable medical device comprises an energy consuming part, and a first energy receiving unit, the first energy receiving unit comprising a first coil configured for receiving wirelessly transferred energy, and a first impedance unit electrically connected to the first coil, the receiving unit being configured to transfer the received energy to the energy consuming part. The implantable medical device further comprises a second energy receiving unit, the second energy receiving unit comprising a second coil configured for receiving wirelessly transferred energy and a second impedance unit electrically connected to the second coil, the receiving unit being configured to transfer the received energy to the energy consuming part. The implantable medical device further comprises a measurement unit configured to measure a parameter related to energy transfer, and a controller configured to control the subcutaneously received energy based on the parameter by controlling the first or the second impedance unit.

According to an embodiment, the first energy receiving unit has a first resonant frequency based on the inductance of the first coil and the impedance of the first impedance unit, and the second energy receiving unit has a second resonant frequency based on the inductance of the second coil and the impedance of second impedance unit.

According to an embodiment, the first receiving unit has a resonant frequency different from the resonant frequency of the second receiving unit.

According to an embodiment, the first and second impedance units are connected in parallel to the respective coil.

According to an aspect, an implantable medical device adapted to receive wirelessly transmitted energy, the implantable medical device comprises an energy consuming part, and a receiving unit configured for receiving wirelessly transferred energy and transferring the received energy to the energy consuming part, the receiving unit comprising a first coil portion and a second coil portion, and a first impedance unit and a second impedance unit, wherein the first impedance unit is connected to the first coil portion and the second impedance unit is connected to the second coil portion. The implantable medical device further comprises a measurement unit configured to measure a parameter related to energy transfer, and a controller configured to control the subcutaneously received energy based on the parameter by controlling the first or the second impedance unit.

According to an embodiment, the first coil portion and the second coil portion are at least one of: portions of the same coil, or portions or different coils connected in series.

According to an embodiment, the first coil portion and the second coil portion have the same inductance, or the first coil portion has a different inductance than the second coil portion.

According to an embodiment, the first impedance is connected in parallel to the first coil portion and the second impedance is connected in parallel to the second coil portion.

According to an embodiment, one of the first coil portion and the second coil portion are overlapping the other of the first coil portion and the second coil portion, or the first coil portion and the second coil portion are not overlapping with the other of the first coil portion and the second coil portion.

According to an embodiment, the first coil portion and the first impedance unit has a first resonance frequency, and the second coil portion and the second impedance unit has a second resonance frequency.

According to an embodiment, the first resonance frequency is different from the second resonance frequency.

According to an embodiment, the first or second impedance unit is a capacitor.

According to an embodiment, the first impedance unit and the second impedance unit have different impedances.

According to an aspect, an implantable medical device adapted to receive wirelessly transmitted energy, the implantable medical device comprises an energy consuming part, and a first receiving unit comprising a first coil configured for receiving wirelessly transferred energy and transferring the received energy to the energy consuming part, and a first impedance electrically connected to the coil. The implantable medical device further comprises a second receiving unit comprising a second coil portion and a third coil portion configured for receiving wirelessly transferred energy and transferring the received energy to the energy consuming part, and a second impedance unit and a third impedance unit, wherein the second impedance unit is connected to the second coil portion and the third impedance unit is connected to the third coil portion. The implantable medical device further comprises a measurement unit configured to measure a parameter related to energy transfer, and a controller configured to control the subcutaneously received energy based on the parameter by controlling the first, the second or the third impedance unit.

A non-therapeutic method of creating a sensation of satiety using a pre-implanted medical device configured to stretch a portion of the stomach wall of a human is further provided. The pre-implanted medical device comprises: a second member configured to be fixated to the stomach wall of the human and comprising a closed curve, a first member configured to be fixated to the stomach wall of the human within the footprint of the second member. The first member is operably connected to the second member such that the first member can be displaced: relative to the second member, and within the footprint of the second member. The non-therapeutic method comprises operating the first member for displacing the first member relative to the second member within the footprint of the second member, for stretching a stomach wall portion between the first and second member, for creating a sensation of satiety.

The non-therapeutic method of creating a feeling of satiety could be linked to a life-style choice of not eating too much or could be linked to a desired physical appearance and as such be linked to physical esthetics.

According to one embodiment, the method further comprises the step of sending a wireless control signal from a wireless remote control to the pre-implanted medical device. The first member is operated as a result of the receipt of the wireless control signal at the pre-implanted medical device.

According to one embodiment, the pre-implanted medical device further comprises a controller configured to control the operation of the first member, and the method comprises operating the first member as a result of at least one of: the receipt of a wireless control signal at the controller of the pre-implanted medical device, the receipt of a sensor signal from a pre-implanted sensor at the controller, and the lapse of a pre-determined time.

A non-therapeutic method of creating a sensation of satiety using a pre-implanted medical device configured to stretch a portion of the stomach wall of a human is further provided. The pre-implanted medical device comprises: a second member configured to be fixated to the stomach wall of the human, an operable first member configured to be fixated to the stomach wall of the human, at least partially within the footprint of the second member, wherein the first member is configured to be operated to deform. The method comprises operating the first member to deform, such that a first portion of the first member is moved away from a first portion of the second member, for stretching a portion of the stomach wall between the first portion of the first member and the first portion of the second member, for creating a sensation of satiety.

The non-therapeutic method of creating a feeling of satiety could be linked to a life-style choice of not eating too much or could be linked to a desired physical appearance and as such be linked to physical esthetics.

According to one embodiment, the method further comprises the step of sending a wireless control signal from a wireless remote control to the pre-implanted medical device. The first member is operated as a result of the receipt of the wireless control signal at the pre-implanted medical device.

According to one embodiment, the pre-implanted medical device further comprises a controller configured to control the operation of the first member, and the method comprises operating the first member as a result of at least one of: the receipt of a wireless control signal at the controller of the pre-implanted medical device, the receipt of a sensor signal from a pre-implanted sensor at the controller, and the lapse of a pre-determined time.

A non-therapeutic method of creating a sensation of satiety using a pre-implanted medical device configured to stretch a portion of the stomach wall of a human is further provided. The pre-implanted medical device comprises: an operable first member configured to be fixated to a first portion of the stomach wall, a second member configured to be fixated to a second portion of the stomach wall, and an operation device. The non-therapeutic method comprises: pulling at least a portion of the first member in a first direction away from at least one portion of the second member, for stretching a portion of the stomach wall between the first portion of the stomach wall and the second portion of the stomach wall for creating the sensation of satiety.

The non-therapeutic method of creating a feeling of satiety could be linked to a life-style choice of not eating too much or could be linked to a desired physical appearance and as such be linked to physical esthetics.

According to one embodiment, the method further comprises the step of sending a wireless control signal from a wireless remote control to the pre-implanted medical device. The first member is operated as a result of the receipt of the wireless control signal at the pre-implanted medical device.

According to one embodiment, the pre-implanted medical device further comprises a controller configured to control the operation of the first member, and the method comprises operating the first member as a result of at least one of: the receipt of a wireless control signal at the controller of the pre-implanted medical device, the receipt of a sensor signal from a pre-implanted sensor at the controller, and the lapse of a pre-determined time.

A non-therapeutic method of creating a sensation of satiety using a pre-implanted medical device configured to stretch a portion of the stomach wall of a human is further provided. The pre-implanted medical device comprises: an operable first member configured to be fixated to a first portion of the stomach wall, a second member configured to be fixated to a second portion of the stomach wall, a mechanical operation device configured to move at least a portion of the first member in a first direction away from at least one portion of the second member. A portion of the mechanical operation device is placed in a remote unit configured to be placed at a remote location in the body of the human, the pre-implanted medical device further comprises a force transferring element configured to transfer mechanical force from the remote unit to the first member. The non-therapeutic method comprises: operating the portion of the mechanical operation device placed in the remote unit such that the force transferring element transfers force from the remote unit to the operable first member for stretching a portion of the stomach wall between the first portion of the stomach wall and the second portion of the stomach wall, for creating the sensation of satiety.

The non-therapeutic method of creating a feeling of satiety could be linked to a life-style choice of not eating too much or could be linked to a desired physical appearance and as such be linked to physical esthetics.

According to one embodiment, the method further comprises the step of sending a wireless control signal from a wireless remote control to the pre-implanted medical device. The portion of the mechanical operation device placed in the remote unit is operated as a result of the receipt of the wireless control signal at the pre-implanted medical device.

According to one embodiment, the pre-implanted medical device further comprises a controller configured to control the operation of the portion of the mechanical operation device placed in the remote unit. The method comprises operating the portion of the mechanical operation device placed in the remote unit as a result of at least one of: the receipt of a wireless control signal at the controller of the pre-implanted medical device, the receipt of a sensor signal from a pre-implanted sensor at the controller, and the lapse of a pre-determined time.

A non-therapeutic method of creating a sensation of satiety using a pre-implanted medical device configured to stretch a portion of the stomach wall of a human is further provided. The pre-implanted medical device comprises: an operable first member configured to be fixated to a first portion of the stomach wall, a second member configured to be fixated to a second portion of the stomach wall, an operable third member configured to be fixated to a third portion of the stomach wall, an operation device, and a mechanical switching device connected to the operation device. The non-therapeutic method comprises: operating the mechanical switching device such that the mechanical switching device switches the operation device from operating the first member to move in a first direction to operating the third member to move in a third direction.

The non-therapeutic method of creating a feeling of satiety could be linked to a life-style choice of not eating too much or could be linked to a desired physical appearance and as such be linked to physical esthetics.

According to one embodiment, the method further comprises the step of sending a wireless control signal from a wireless remote control to the pre-implanted medical device. The mechanical switching device is operated as a result of the receipt of the wireless control signal at the pre-implanted medical device.

According to one embodiment, the pre-implanted medical device further comprises a controller configured to control the operation of the mechanical switching device. The method comprises operating the mechanical switching device as a result of at least one of: the receipt of a wireless control signal at the controller of the pre-implanted medical device, the receipt of a sensor signal from a pre-implanted sensor at the controller, and the lapse of a pre-determined time.

A non-therapeutic method of creating a sensation of satiety using a pre-implanted medical device configured to stretch a portion of the intestinal wall of a human is further provided. The pre-implanted medical device comprises: an operable first member configured to be fixated to a first portion of the intestinal wall and an operation device connected to the operable first member. The non-therapeutic method comprising altering the operable first member such that the intestinal wall is actively stretched for creating a sensation of satiety.

According to one embodiment, the operable first member comprises at least one curved portion configured to be fixated to the first portion of the intestinal wall. The method comprises operating the operable first member for altering the curvature of the operable first member for actively stretching the first portion of the intestinal wall.

According to one embodiment, the operable first member is configured to be fixated to the first portion of the intestinal wall at a plurality of positions. The method comprises operating the operable first member for altering the operable first member by increasing a length of the operable first member for actively stretching the intestinal wall.

According to one embodiment, the operable first member comprises a closed curve. The method comprises operating the operable first member for altering the operable first member by increasing the length of the closed curve.

According to one embodiment, the method comprises operating the operable first member for altering the volume of the first member.

According to one embodiment, the method further comprises the step of sending a wireless control signal from a wireless remote control to the pre-implanted medical device. The operable first member is operated as a result of the receipt of the wireless control signal at the pre-implanted medical device.

According to one embodiment, the pre-implanted medical device further comprises a controller configured to control the operation of the operable first member. The method further comprises operating the operable first member as a result of at least one of: the receipt of a wireless control signal at the controller of the pre-implanted medical device, the receipt of a sensor signal from a pre-implanted sensor at the controller, and the lapse of a pre-determined time.

A method of creating a sensation of satiety using a pre-implanted medical device configured to stretch a portion of the intestinal wall of a human is further provided. The pre-implanted medical device comprises: an operable first member configured to be fixated to a first portion of the intestinal wall, a second member configured to be fixated to a second portion of the intestinal wall, and an operation device connected to the operable first member. The method comprises operating the operable first member by moving at least a portion of the operable first member in a first direction away from the second member, for stretching the intestinal wall between the first and second portions of the intestinal wall.

According to one embodiment, the step of operating the operable first member by moving at least a portion of the operable first member in a first direction away from the second member comprises operating the operable first member by moving at least a portion of the operable first member in a first direction away from the second member substantially in an axial direction of the intestine.

According to one embodiment, the method further comprises the step of sending a wireless control signal from a wireless remote control to the pre-implanted medical device. The operable first member is operated as a result of the receipt of the wireless control signal at the pre-implanted medical device. The pre-implanted medical device may further comprise a controller configured to control the operation of the operable first member. The method may further comprise operating the operable first member as a result of at least one of: the receipt of a wireless control signal at the controller of the pre-implanted medical device, the receipt of a sensor signal from a pre-implanted sensor at the controller, and the lapse of a pre-determined time.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane exceeds one half of the length of a circumference of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is be configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane is less than one half of the length of a circumference of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member exceeds an area of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member is less than an area of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane exceeds one half of the length of a circumference of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is be configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane is less than one half of the length of a circumference of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member exceeds an area of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device.

The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

According to one embodiment, a medical device for actively stretching a stomach or intestinal wall of a human for creating a sensation of satiety is provided. The medical device is implantable and comprises an operable first member, a second member and an operation device. The operable first member is configured to be directly or indirectly fixated to a first portion of the stomach or intestinal wall, and is configured to be invaginated and held in place by tissue-to-tissue sutures or staplers. The second member is configured to be directly or indirectly fixated to a second portion of the stomach or intestinal wall, and is configured to be invaginated and held in place by tissue-to-tissue sutures or staplers. The operation device is configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member is less than an area of the portion of the operable first member positioned in the stretching plane.

According to one embodiment, the portion of the operable first member positioned in the stretching plane is configured to be invaginated.

According to one embodiment, the operation device is configured to move at least a portion of the operable first member by deforming the operable first member.

According to one embodiment, the operation device is configured to move at least a portion of the operable first member by pulling at least a portion of the first member in a first direction away from at least one portion of the second member.

According to one embodiment, at least a portion of the operation device is placed in a remote unit configured to be placed at a remote location in the body of the patient.

According to one embodiment, the operable first member is mechanically operable, and the operation device is a mechanical operation device. The mechanical operation device may comprise a pulling member for operating the operable first member. The pulling member may comprise at least one flexible element, directly or indirectly fixated to the first member, wherein the flexible element is configured to operate the first member by pulling at least a portion of the first member.

According to one embodiment, the operable first member is hydraulically operable, and the operation device is a hydraulic operation device. The pulling member may comprise at least one hydraulic actuator, directly or indirectly fixated to at least one of the first and second members.

According to one embodiment, the operation device is a piezoelectric operation device. The piezoelectric operation device may comprise a piezoelectric motor.

According to one embodiment, the medical device further comprises a controller for controlling the operation device. The controller may be placed in the remote unit. The controller may comprise a wireless transceiver for communicating wirelessly with a source external to the body of the patient.

According to one embodiment, the medical device further comprises at least one sensor configured to sense a physical parameter of the medical device system. The sensor may be a sensor configured to sense at least one of: a temperature of the medical device system, a parameter related to the power consumption of the medical device system, a parameter related to strain in at least one of the first and second members, a parameter related to a status of the energy storage unit, a parameter related to the wireless transfer of energy from a source external to the body of the patient, a hydraulic pressure in at least one of the hydraulic actuator, the hydraulic pump, the hydraulic reservoir and the hydraulic conduit.

In all embodiments, the at least one electrode can be used for exercising the muscle tissue that is mechanically affected by the members, or for increasing the satiety-creating effect of the stretching by further stimulating the stretch receptors of the stomach wall or intestine. Stimulation electrodes could be replaced of complemented with a vibrator configured to mechanically stimulate a wall portion of the stomach or intestine, such that the wall of the stomach is actively stretched and mechanically stimulated simultaneously for creating a sensation of satiety. The vibrator may be operated by a piezoelectric operation device.

The electrode or vibrator may be connected to a wireless energy receiver configured to receive energy for powering the electrode or vibrator wirelessly.

The member including the electrode and/or vibrator may be configured to be at least partially invaginated by the tissue of the stomach or intestine wall using sutures or staplers.

The vibrator may be configured to vibrate at a frequency in the range of 1-100 Hz, such as in the range of 1-20 Hz, and with a period of 0.01-1 second, such as of 0.05-1 seconds. The vibrator may be configured to vibrate at an amplitude such that the tissue in the stomach wall is displaced at least 1 mm.

The member may further comprise a second electrode, and the first and second electrodes may be configured for successively electrically stimulating two portions of the tissue wall of the stomach or intestine. The first and second electrodes may be placed on the member at a distance from each other being at least 5 mm, and/or a distance from each other corresponding to a wavelength of a wave pattern of a movement of smooth muscles of the stomach wall or intestine, or by a multiple of such a wavelength.

The stimulation controller may be configured to send stimulation signals to the first and second electrode in pulses, or pulse trains. And the pulses or pulse trains may have a frequency being determined based on a frequency of the wave pattern of a movement of smooth muscles of the stomach or intestine. The stimulation controller is configured to send stimulation signals to the first and second electrode in pulses or pulse trains having a pulse separation or pulse train separation of 0,5-10 s, such as 1-8 s, such as 2-6 s.

The medical devices addorcing to any of the embodiments described herein could be implanted using a laparoscopic surgical technique which could comprise the following steps:

introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,

blowing in pressurized gas through the needle or trocar or surgical instrument or any device inflating the abdominal cavity,

introducing working instruments comprising at least one of the steps of,

inserting at least one first trocar into the abdominal cavity,

introducing at least one camera through the trocar,

inserting at least a second trocar into the abdominal cavity,

inserting at least one instrument preferably through the second trocar,

inserting at least a third trocar into the abdominal cavity,

inserting at least one second instrument preferably through the third trocar,

dissecting an area of the stomach or intestine,

identifying a suitable position for placing a stretching device on the stomach or intestinal wall,

placing a control device in the body for controlling the stretching device,

inserting the medical device into the abdomen of the patient,

fixating the operable first member to a first portion of the stomach or intestinal wall,

fixating the second member to a second portion of the stomach or intestinal wall,

placing the control unit, and

extracting the instruments, camera and trocar, and in relation thereto

suturing, if necessary, the abdominal wall and permanently closing the skin.

The medical devices addorcing to any of the embodiments described herein could be implanted using an open surgical technique which could comprise the following steps:

cutting and open the skin and abdominal wall.

dissecting or cutting through the fascia of at least one of any muscles or any fibrotic intersections comprising; the rectus abdominalis, transversus abdominalis, the External Oblique, the Internal Oblique, the linea alba, a tendnous intersection, and the umbilicus,

introducing at least one working instrument,

dissecting an area of the stomach,

identifying a suitable position for placing a stretching device on the stomach wall,

placing a control device in the body for controlling the stretching device,

inserting the medical device into the abdomen of the patient,

fixating the operable first member to a first portion of the stomach wall,

fixating the second member to a second portion of the stomach wall,

placing the control unit, and

extracting the instruments, camera and trocar, and in relation thereto

suturing, if necessary, the abdominal wall and permanently closing the skin.

Any embodiment, part of embodiment, method, or part of method may be combined in any applicable way.

### BRIEF DESCRIPTION OF THE DRAWINGS

The invention is now described, by way of example, with reference to the accompanying drawing, in which:

FIG. 1a shows an embodiment of a medical device for actively stretching a stomach wall of a patient for creating a sensation of satiety, when placed on a stomach.

FIG. 1b shows an embodiment of a medical device for actively stretching a stomach wall of a patient for creating a sensation of satiety, in a plain top view.

FIG. 1b′ shows an embodiment of a medical device for actively stretching a stomach wall of a patient for creating a sensation of satiety, when operated to stretch the stomach wall, in a plain top view.

FIG. 1c shows an embodiment of a medical device for actively stretching a stomach wall of a patient for creating a sensation of satiety, in a folded state in a plain side view.

FIG. 2a shows an embodiment of a medical device for actively stretching a stomach wall of a patient for creating a sensation of satiety, when placed on a stomach.

FIG. 2b shows an embodiment of a medical device for actively stretching a stomach wall of a patient for creating a sensation of satiety, in a plain top view.

FIG. 3a shows an embodiment of a medical device for actively stretching a stomach wall of a patient for creating a sensation of satiety, when placed on a stomach.

FIG. 3b shows an embodiment of a medical device for actively stretching a stomach wall of a patient for creating a sensation of satiety, in a plain top view.

FIG. 4a shows an embodiment of a medical device for actively stretching a stomach wall of a patient for creating a sensation of satiety, when placed on a stomach.

FIG. 4b shows an embodiment of a medical device for actively stretching a stomach wall of a patient for creating a sensation of satiety, in a plain top view.

FIG. 4c shows an embodiment of a medical device for actively stretching a stomach wall of a patient for creating a sensation of satiety, in a folded state in a plain side view.

FIG. 5 shows an embodiment of a medical device for actively stretching a stomach wall of a patient for creating a sensation of satiety, when operated to stretch the stomach wall, in a plain top view.

FIG. 6 shows an embodiment of a medical device for actively stretching a stomach wall of a patient for creating a sensation of satiety, when operated to stretch the stomach wall, in a plain top view.

FIG. 7 shows an embodiment of a remotely powered medical device for actively stretching a stomach wall of a patient for creating a sensation of satiety, when operated to stretch the stomach wall, in a plain top view.

FIG. 8 shows an embodiment of a remotely powered medical device for actively stretching a stomach wall of a patient for creating a sensation of satiety, in a folded state in a plain top view.

FIG. 9 shows an embodiment of a remotely powered medical device for actively stretching a stomach wall of a patient for creating a sensation of satiety, when operated to stretch the stomach wall, in a plain top view.

FIG. 10 shows an embodiment of a medical device for actively stretching a stomach wall of a patient for creating a sensation of satiety, when placed on a stomach and partially invaginated by the stomach wall.

FIG. 11 shows an embodiment of a remotely powered medical device for actively stretching a stomach wall of a patient for creating a sensation of satiety, when operated to stretch the stomach wall, in a plain top view.

FIG. 12a shows an embodiment of a remotely powered medical device for actively stretching a stomach wall of a patient for creating a sensation of satiety, when operated to stretch the stomach wall, in a plain top view.

FIG. 12b shows an embodiment of a block and tackle functionality.

FIG. 12c shows an embodiment of a block and tackle functionality.

FIG. 12d shows an embodiment of a block and tackle functionality.

FIG. 12e shows an embodiment of a block and tackle functionality.

FIG. 13a shows an embodiment of a remotely powered medical device for actively stretching a stomach wall of a patient for creating a sensation of satiety, in an elevated perspective view from the right.

FIG. 13b shows an embodiment of a remotely powered medical device for actively stretching a stomach wall of a patient for creating a sensation of satiety, in a folded state in a plain top view.

FIG. 14a shows an embodiment of a remotely powered medical device for actively stretching a stomach wall of a patient for creating a sensation of satiety, in an elevated perspective view from the right.

FIG. 14a′ shows an embodiment of a remotely powered medical device for actively stretching a stomach wall of a patient for creating a sensation of satiety, in a folded state in a plain top view.

FIG. 14b shows an embodiment of a remotely powered medical device for actively stretching a stomach wall of a patient for creating a sensation of satiety, in an elevated perspective view from the right.

FIG. 14c shows an embodiment of a remotely powered medical device for actively stretching a stomach wall of a patient for creating a sensation of satiety, in an elevated perspective view from the right.

FIG. 14c′ shows an embodiment of a medical device for actively stretching a stomach wall of a patient for creating a sensation of satiety, when placed on a stomach.

FIG. 14d shows an embodiment of a remotely powered medical device for actively stretching a stomach wall of a patient for creating a sensation of satiety, in an elevated perspective view from the right.

FIG. 14d′ shows an embodiment of a medical device for actively stretching a stomach wall of a patient for creating a sensation of satiety, when placed on a stomach.

FIG. 15 shows an embodiment of a remotely powered medical device for actively stretching a stomach wall of a patient for creating a sensation of satiety, when operated to stretch the stomach wall, in a plain top view.

FIG. 16 shows an embodiment of a medical device for actively stretching a stomach wall of a patient for creating a sensation of satiety, when placed on a stomach and partially invaginated by the stomach wall.

FIG. 16′ shows an embodiment of a medical device for actively stretching a stomach wall of a patient for creating a sensation of satiety, when placed on a stomach and completely invaginated by the stomach wall.

FIG. 17a shows an embodiment of a medical device for actively stretching a stomach wall of a patient for creating a sensation of satiety, when operated to stretch the stomach wall, in a plain top view.

FIG. 17a′ shows an embodiment of a medical device for actively stretching a stomach wall of a patient for creating a sensation of satiety, when operated to stretch the stomach wall, in a plain top view.

FIG. 17b shows an embodiment of a medical device for actively stretching a stomach wall of a patient for creating a sensation of satiety, in a cross-sectional side view.

FIG. 17b′ shows an embodiment of a medical device for actively stretching a stomach wall of a patient for creating a sensation of satiety, in a cross-sectional side view.

FIG. 18a shows an embodiment of a medical device for actively stretching a stomach wall of a patient for creating a sensation of satiety, in a first state, in a plain top view.

FIG. 18b shows an embodiment of a medical device for actively stretching a stomach wall of a patient for creating a sensation of satiety, in the first state, in a cross-sectional side view.

FIG. 19a shows an embodiment of a medical device for actively stretching a stomach wall of a patient for creating a sensation of satiety, in a second state, in a plain top view.

FIG. 19b shows an embodiment of a medical device for actively stretching a stomach wall of a patient for creating a sensation of satiety, in the second state, in a cross-sectional side view.

FIG. 20a shows an embodiment of a medical device for actively stretching a stomach wall of a patient for creating a sensation of satiety, in a third state, in a plain top view.

FIG. 20b shows an embodiment of a medical device for actively stretching a stomach wall of a patient for creating a sensation of satiety, in the third state, in a cross-sectional side view.

FIG. 21 shows an embodiment of a medical device for actively stretching a stomach wall of a patient for creating a sensation of satiety, when placed on a stomach and partially invaginated by the stomach wall.

FIG. 22 shows an embodiment of a medical device for actively stretching a stomach wall of a patient for creating a sensation of satiety, in a plain top view.

FIG. 23 shows an embodiment of a medical device for actively stretching a stomach wall of a patient for creating a sensation of satiety, in a plain top view.

FIG. 24 shows an embodiment of a medical device for actively stretching a stomach wall of a patient for creating a sensation of satiety, in a cross-sectional top view.

FIG. 24 shows an embodiment of a medical device for actively stretching a stomach wall of a patient for creating a sensation of satiety, when operated for stretching the stomach wall, in a cross-sectional top view.

FIG. 25 shows an embodiment of a medical device for actively stretching a stomach wall of a patient for creating a sensation of satiety, when operated for stretching the stomach wall, in a cross-sectional top view.

FIG. 26 shows an embodiment of a medical device for actively stretching a stomach wall of a patient for creating a sensation of satiety, when operated for stretching the stomach wall, in a cross-sectional top view.

FIG. 27 shows an embodiment of a medical device for actively stretching a stomach wall of a patient for creating a sensation of satiety, when operated for stretching the stomach wall, in a cross-sectional top view.

FIG. 28a shows an embodiment of a medical device for actively stretching a stomach wall of a patient for creating a sensation of satiety, in plain top view.

FIG. 28b shows an embodiment of a medical device for actively stretching a stomach wall of a patient for creating a sensation of satiety, when operated for stretching the stomach wall, in a plain top view.

FIG. 28c shows an embodiment of a medical device for actively stretching a stomach wall of a patient for creating a sensation of satiety, when operated for stretching the stomach wall, in a plain top view.

FIG. 29a shows an embodiment of a medical device for actively stretching a stomach wall of a patient for creating a sensation of satiety, in plain top view.

FIG. 29b shows an embodiment of a medical device for actively stretching a stomach wall of a patient for creating a sensation of satiety, when operated for stretching the stomach wall, in a plain top view.

FIG. 29c shows an embodiment of a medical device for actively stretching a stomach wall of a patient for creating a sensation of satiety, when operated for stretching the stomach wall, in a plain top view.

FIG. 30a shows an embodiment of a medical device for actively stretching a stomach wall of a patient for creating a sensation of satiety, in plain top view.

FIG. 30b shows an embodiment of a medical device for actively stretching a stomach wall of a patient for creating a sensation of satiety, when operated for stretching the stomach wall, in a plain top view.

FIG. 30c shows an embodiment of a medical device for actively stretching a stomach wall of a patient for creating a sensation of satiety, when operated for stretching the stomach wall, in a plain top view.

FIG. 31A shows an embodiment of a medical device for actively stretching a stomach wall of a patient for creating a sensation of satiety, in plain top view.

FIG. 31B shows an embodiment of a medical device for actively stretching a stomach wall of a patient for creating a sensation of satiety, when operated for stretching the stomach wall, in a plain top view.

FIG. 31C shows an embodiment of a medical device for actively stretching a stomach wall of a patient for creating a sensation of satiety, in plain top view.

FIG. 31D shows an embodiment of a medical device for actively stretching a stomach wall of a patient for creating a sensation of satiety, in an idle state, in plain top view.

FIG. 31E shows the medical device of FIG. 31D, in a stretching state, in plain top view.

FIG. 31F shows an embodiment of a medical device for actively stretching a stomach wall of a patient for creating a sensation of satiety, in plain top view.

FIG. 31G shows an embodiment of a medical device for actively stretching a stomach wall of a patient for creating a sensation of satiety, in a stretching state, in plain top view.

FIG. 31H shows an embodiment of a medical device for actively stretching a stomach wall of a patient for creating a sensation of satiety, in a stretching state, in plain top view.

FIG. 311 shows an embodiment of a medical device for actively stretching a stomach wall of a patient for creating a sensation of satiety, in a stretching state, in plain top view.

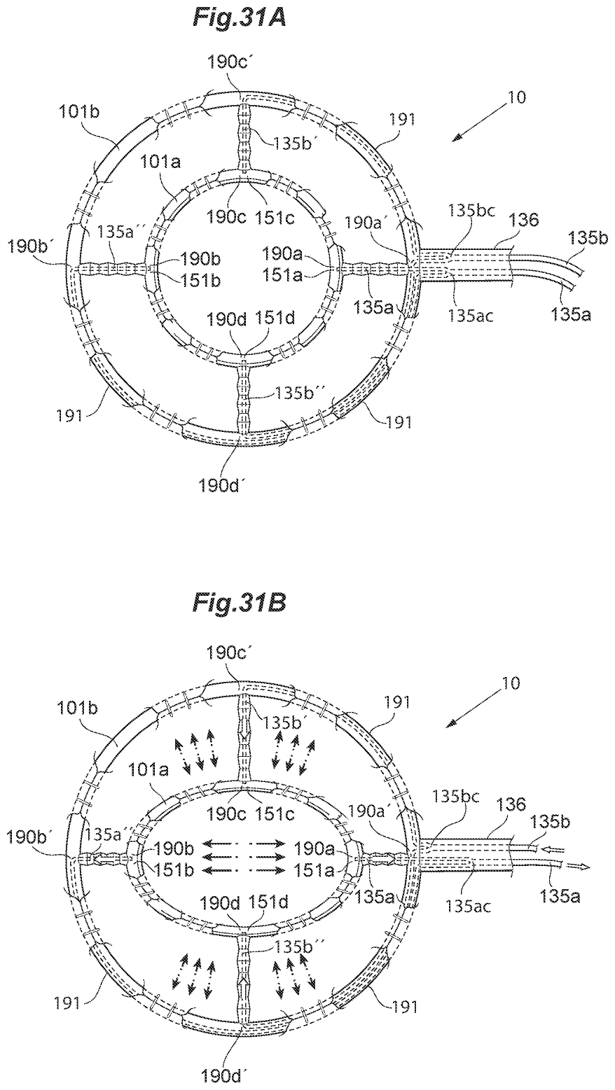

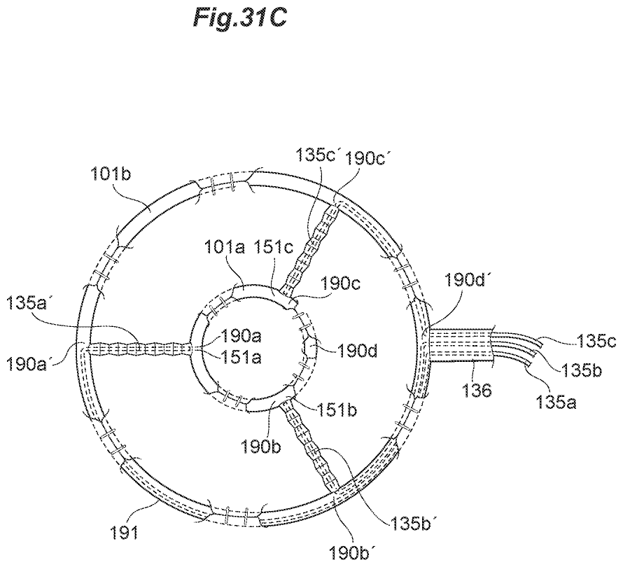

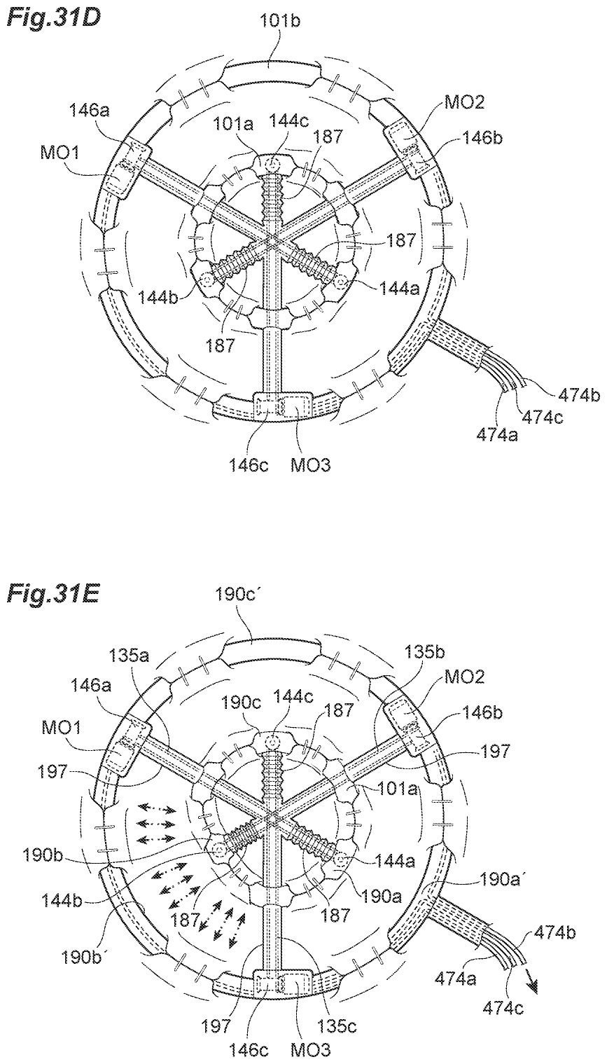

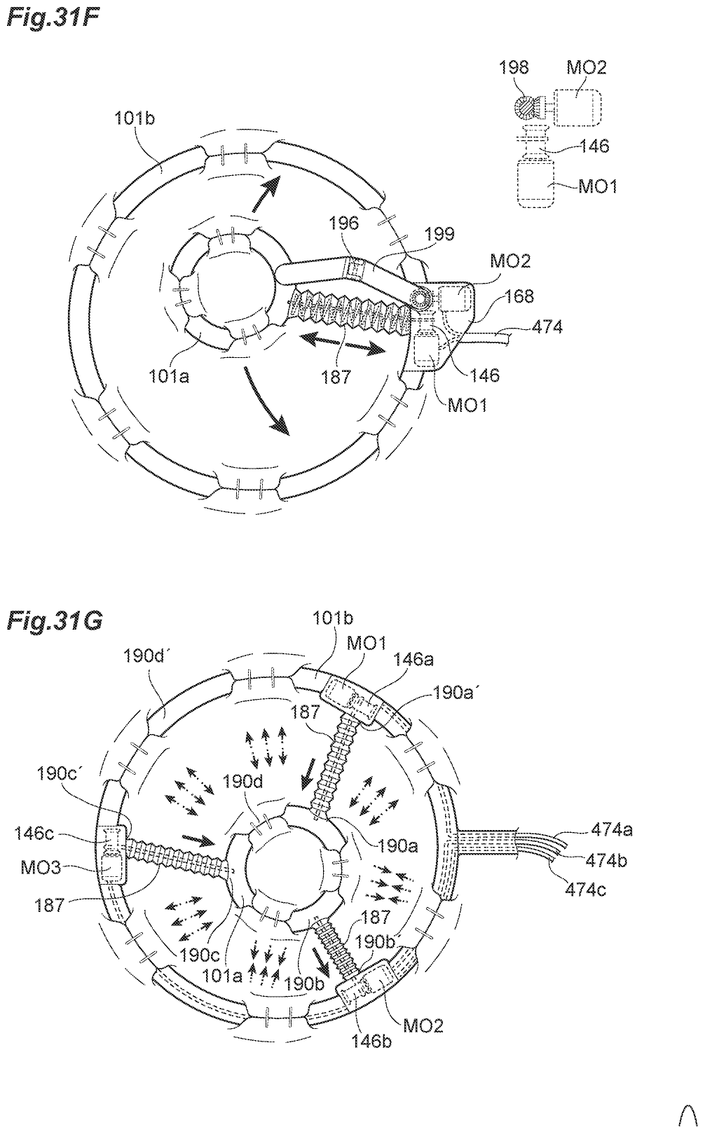

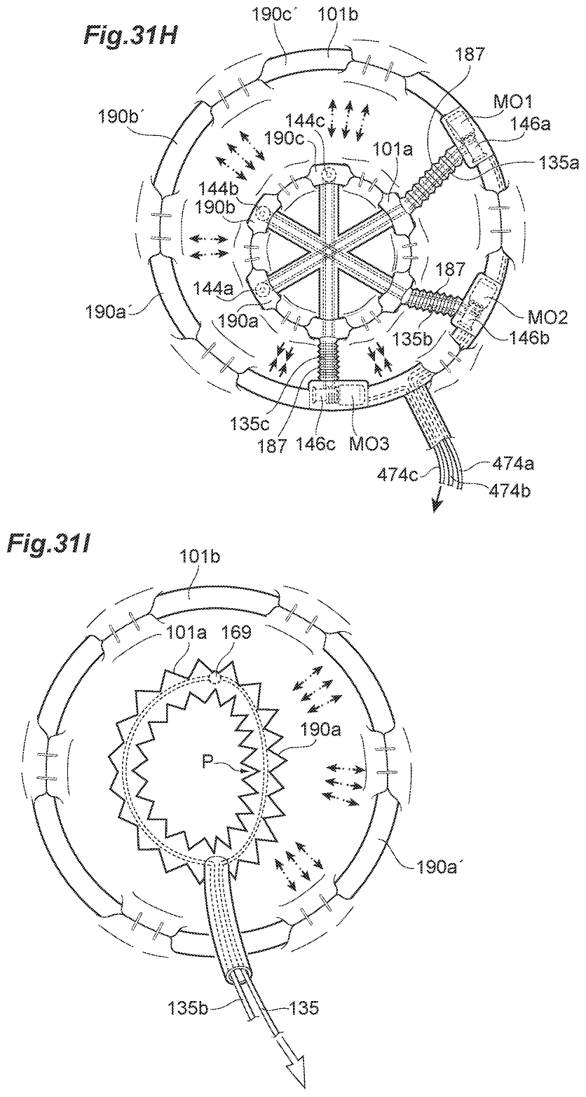

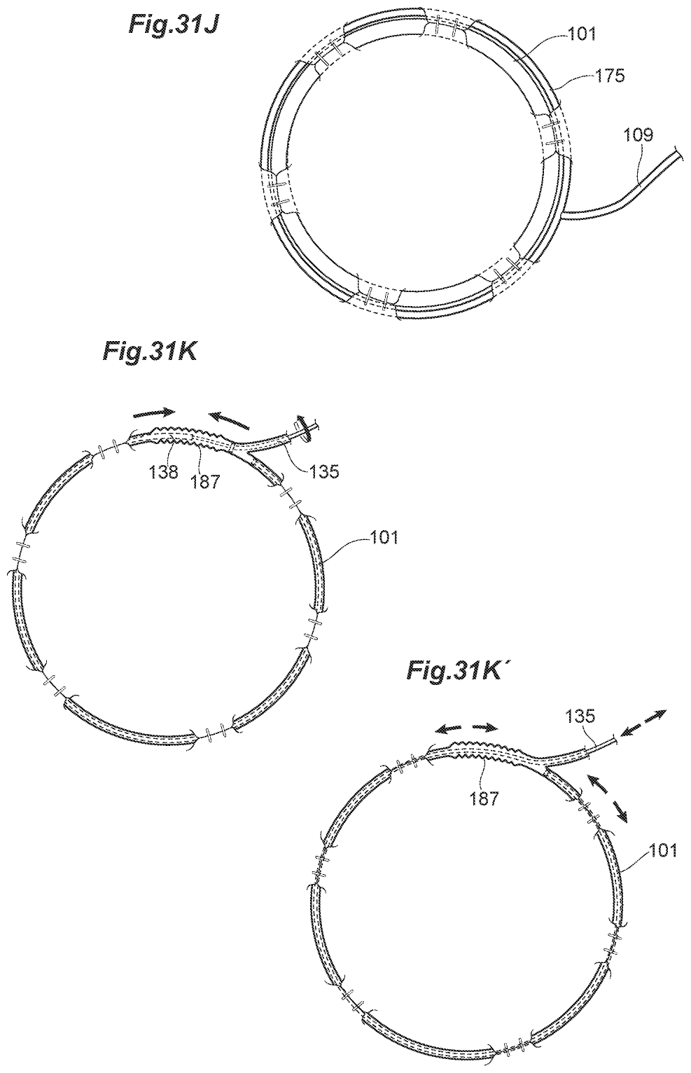

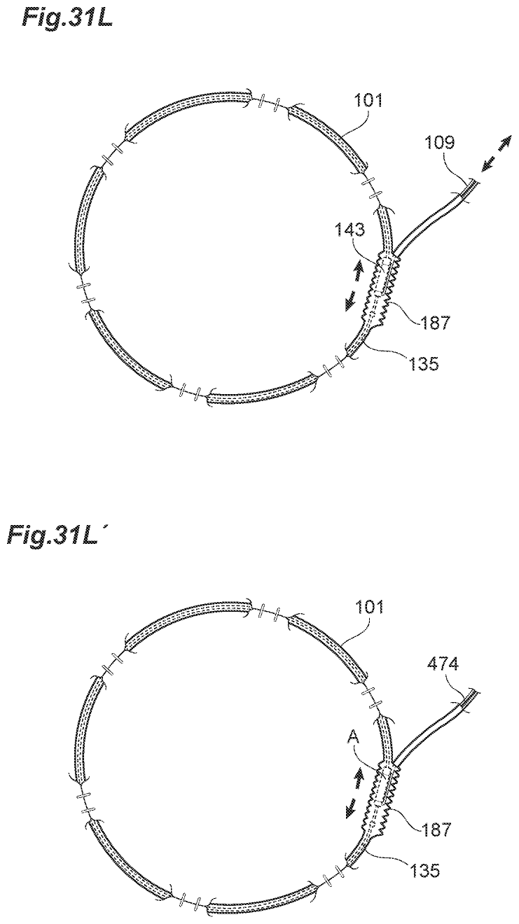

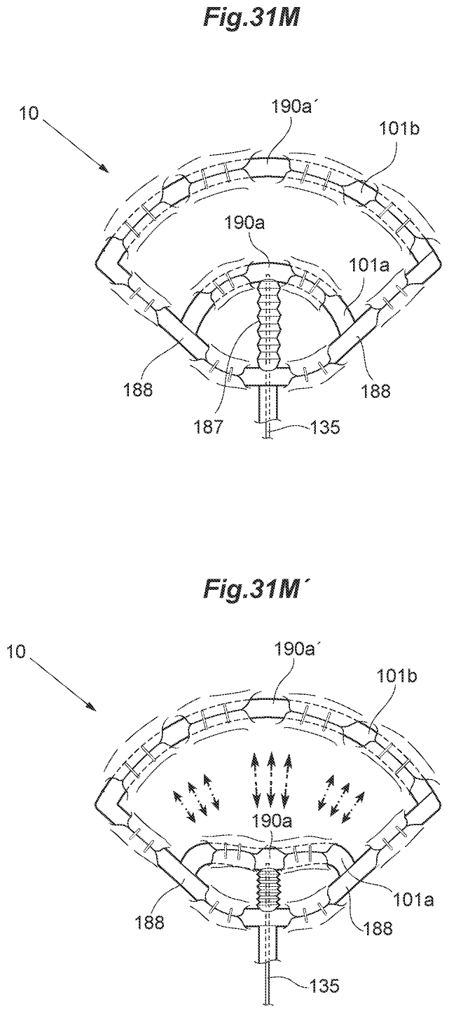

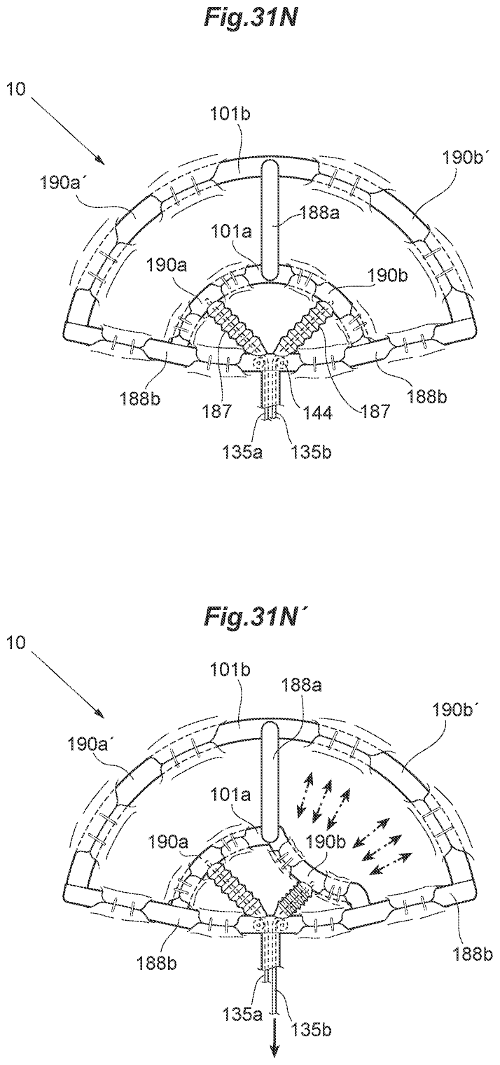

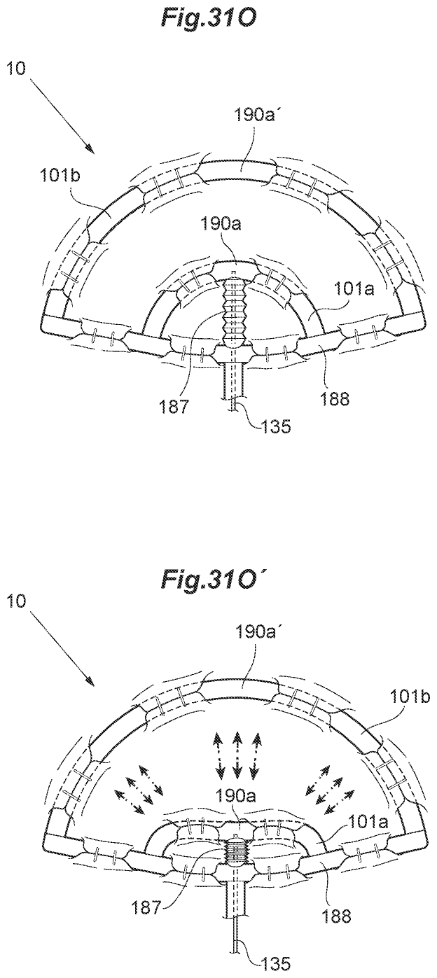

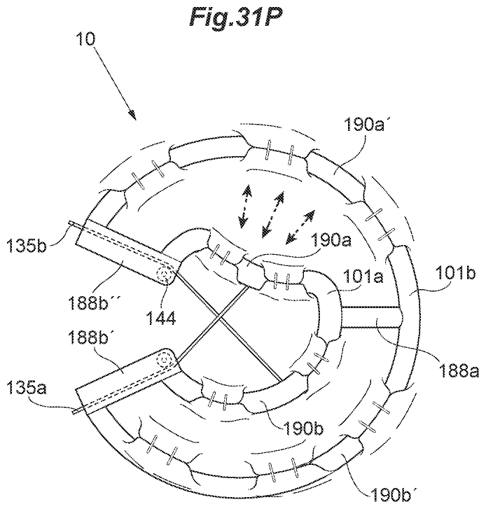

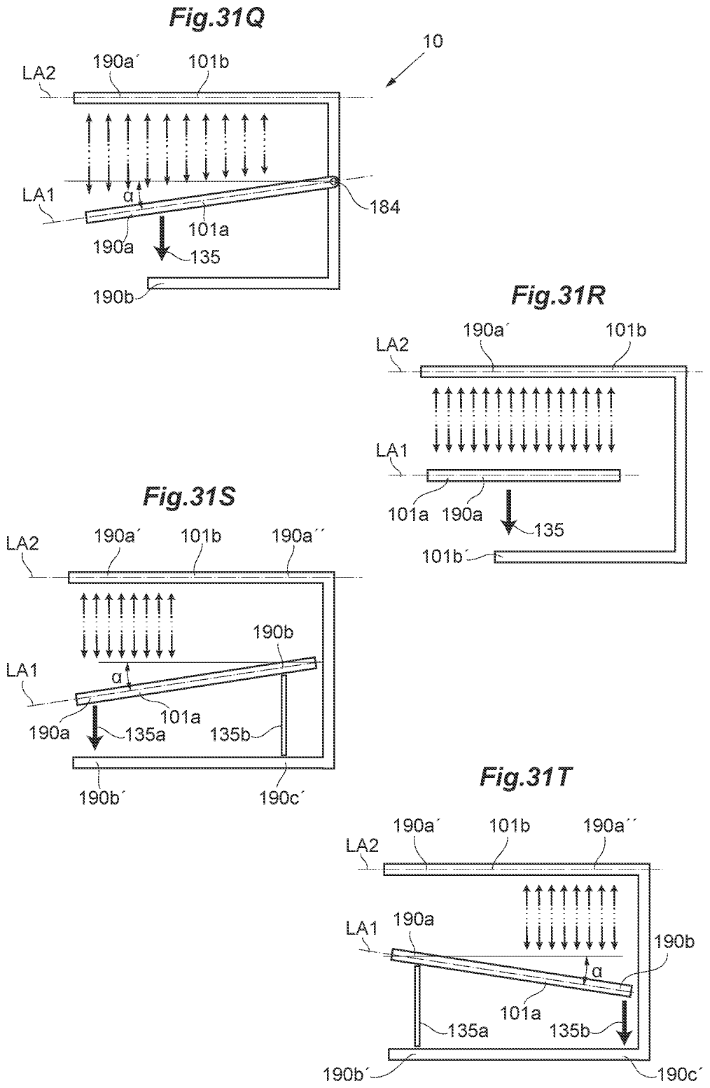

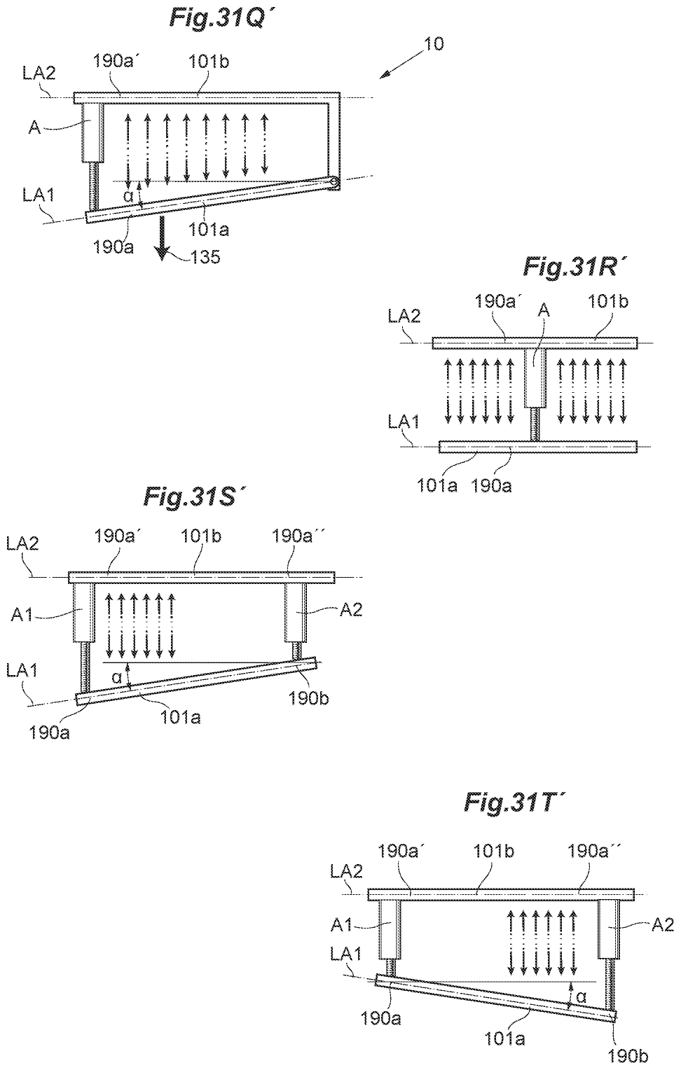

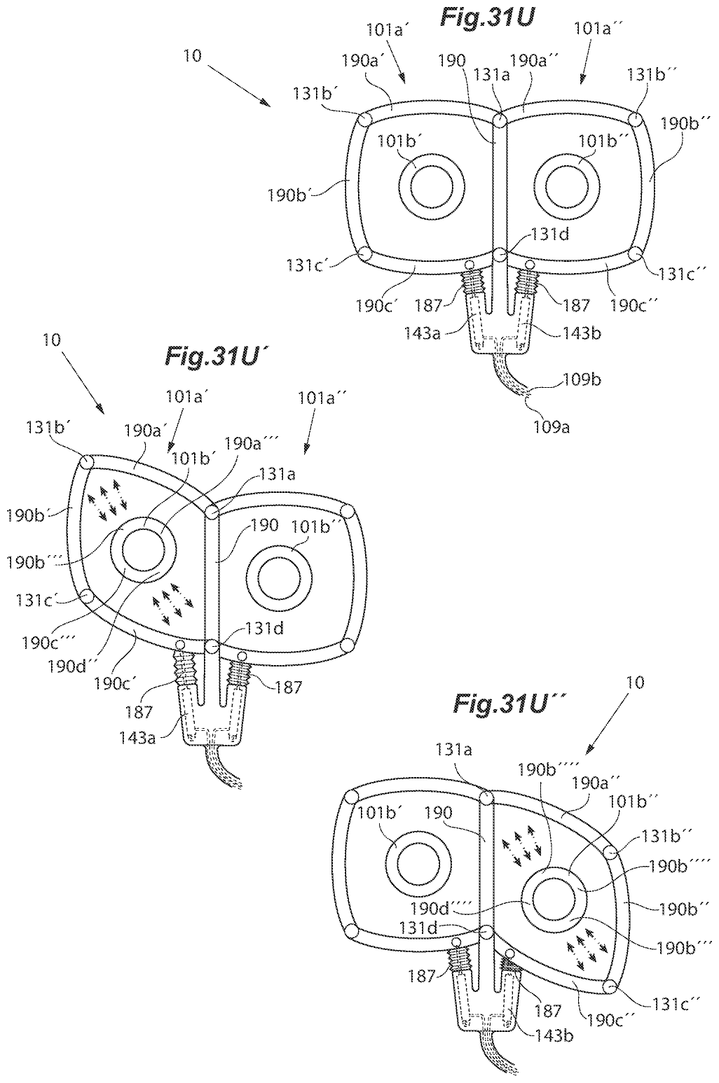

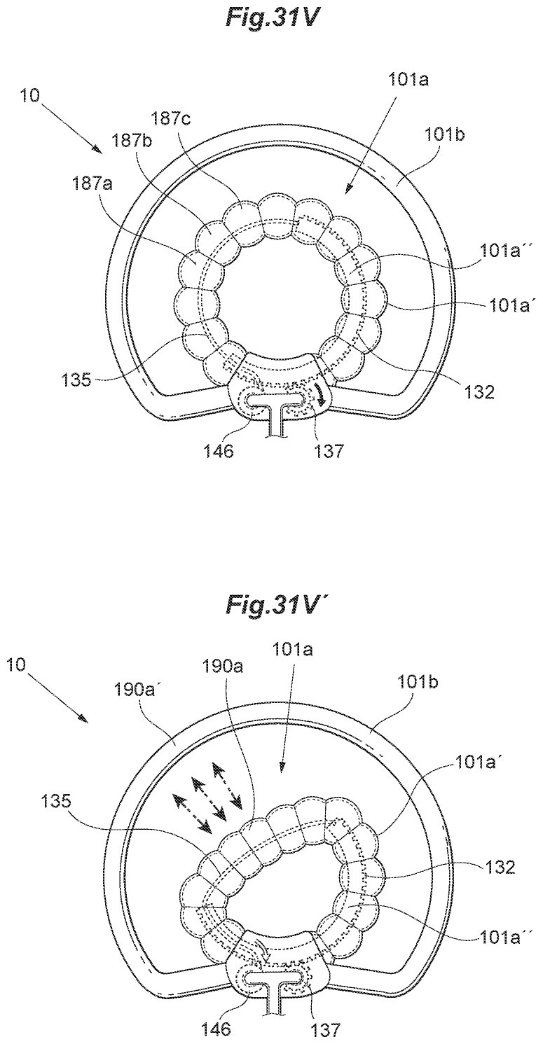

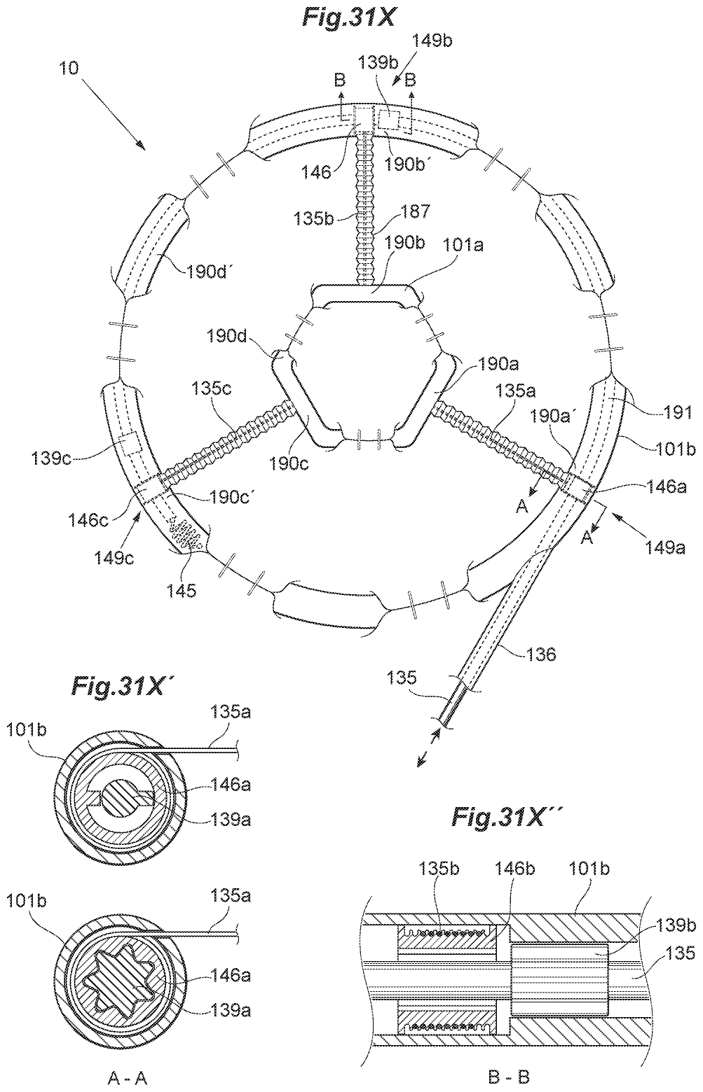

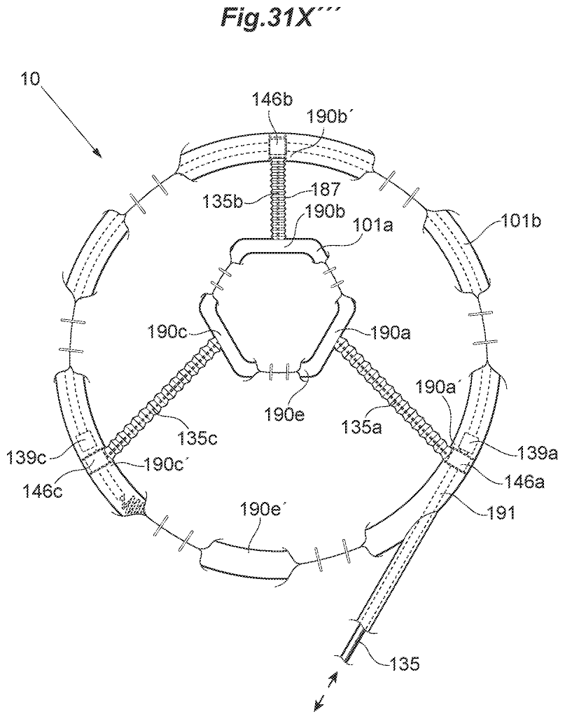

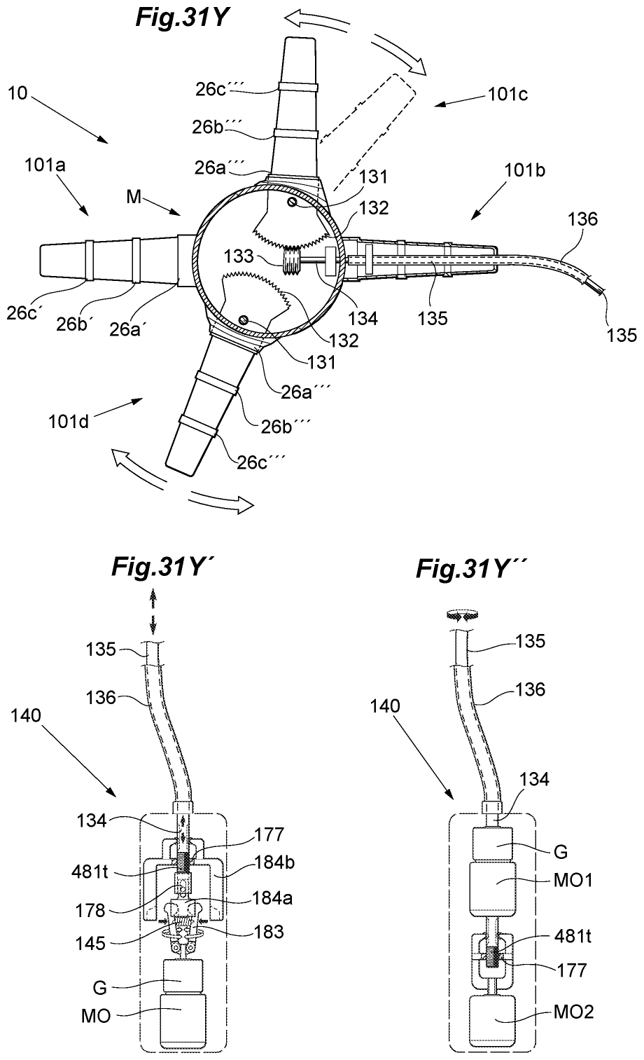

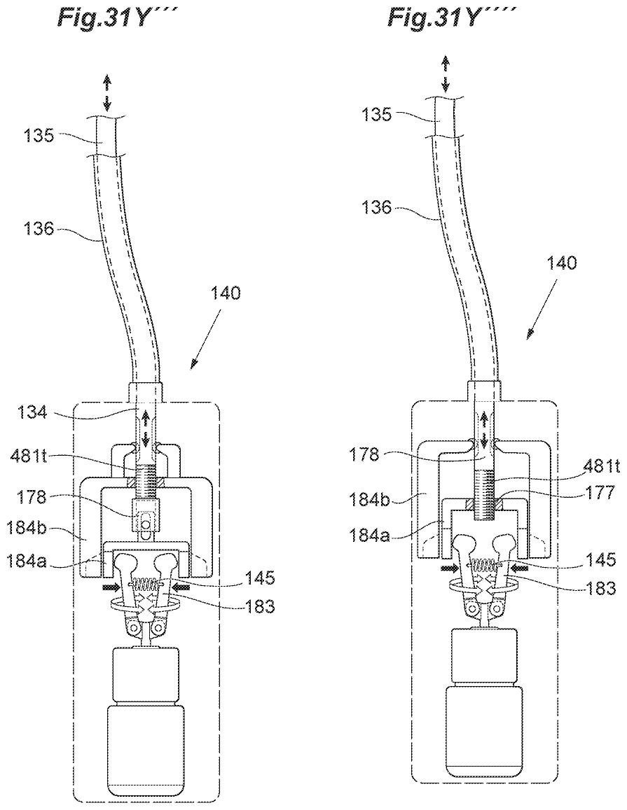

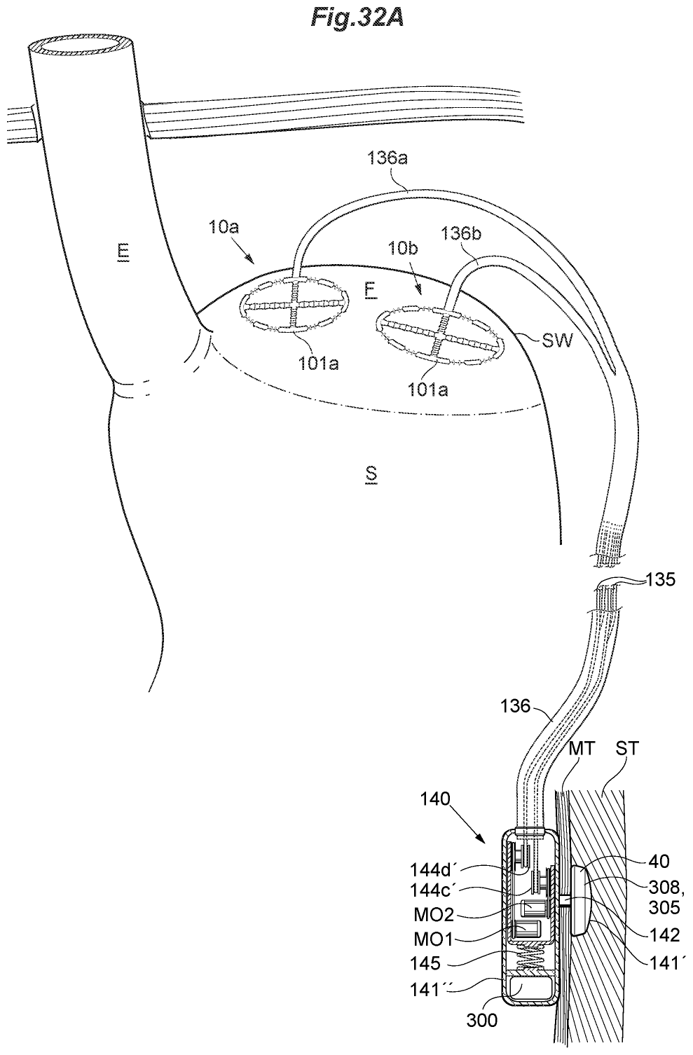

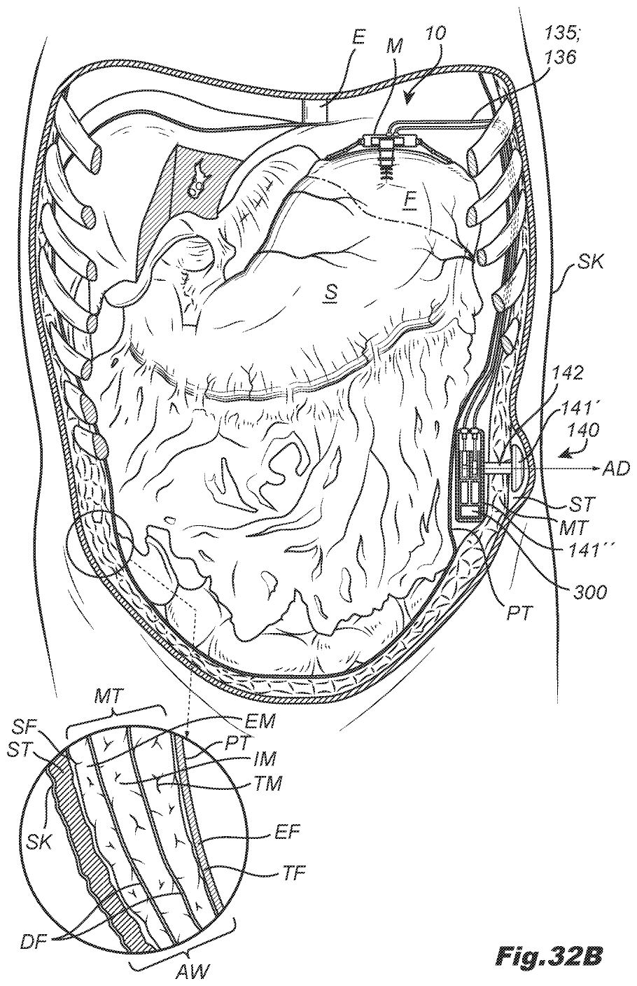

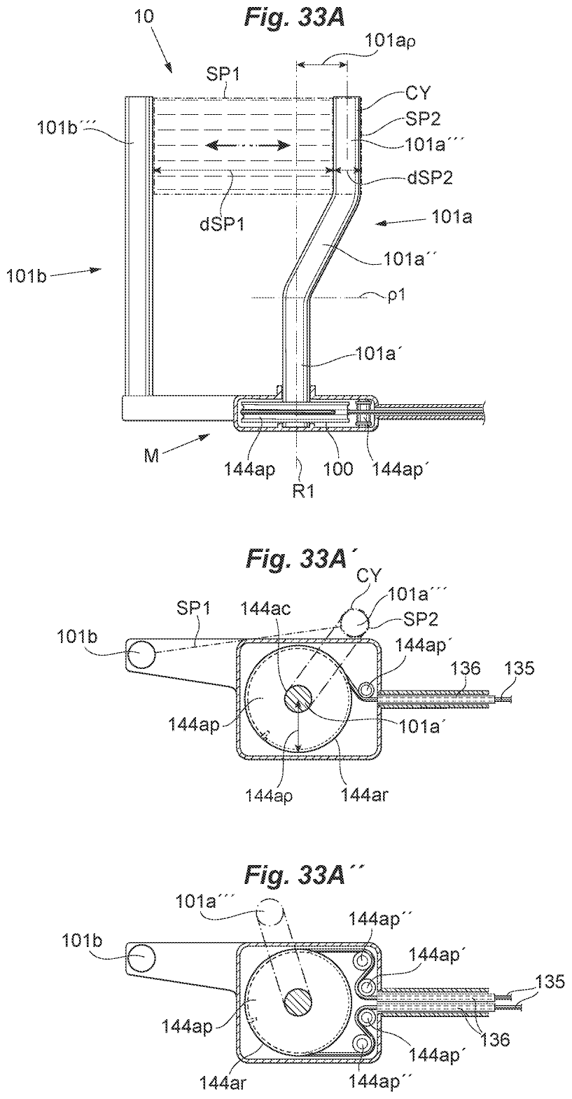

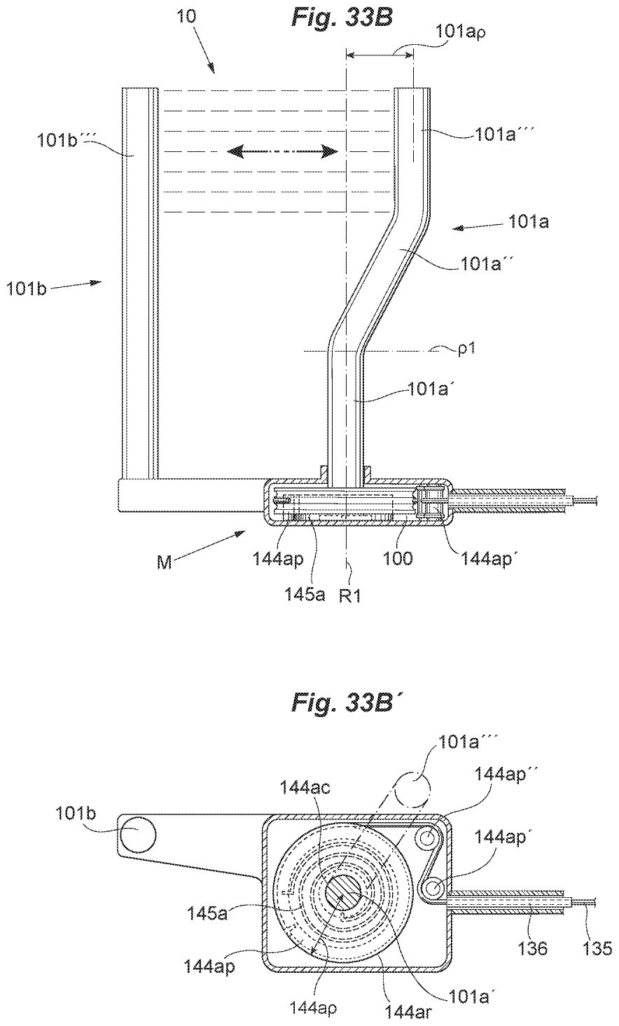

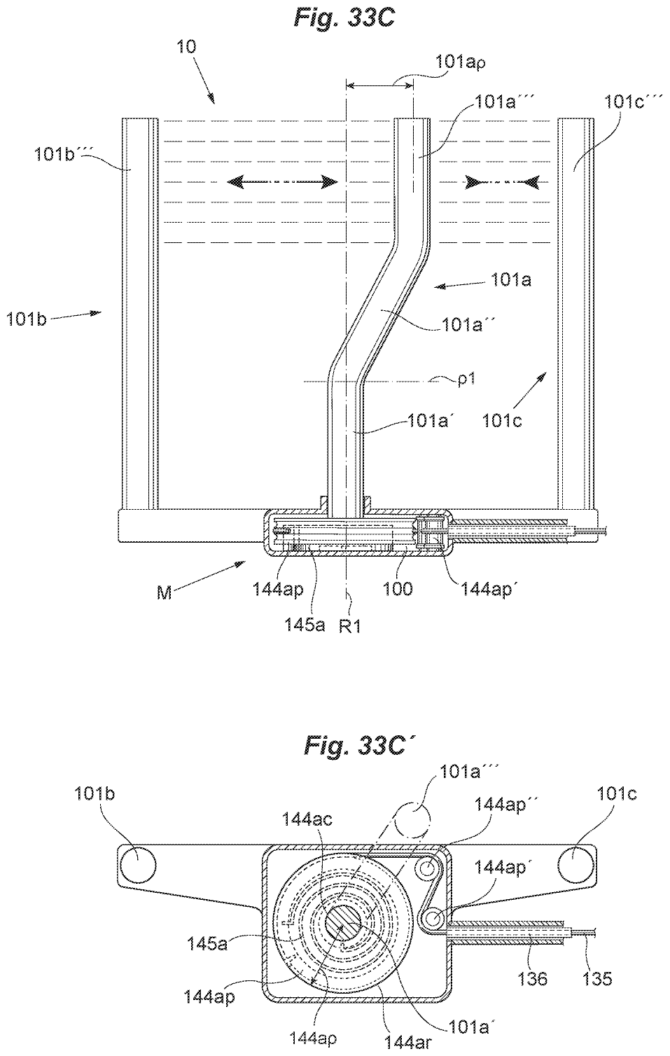

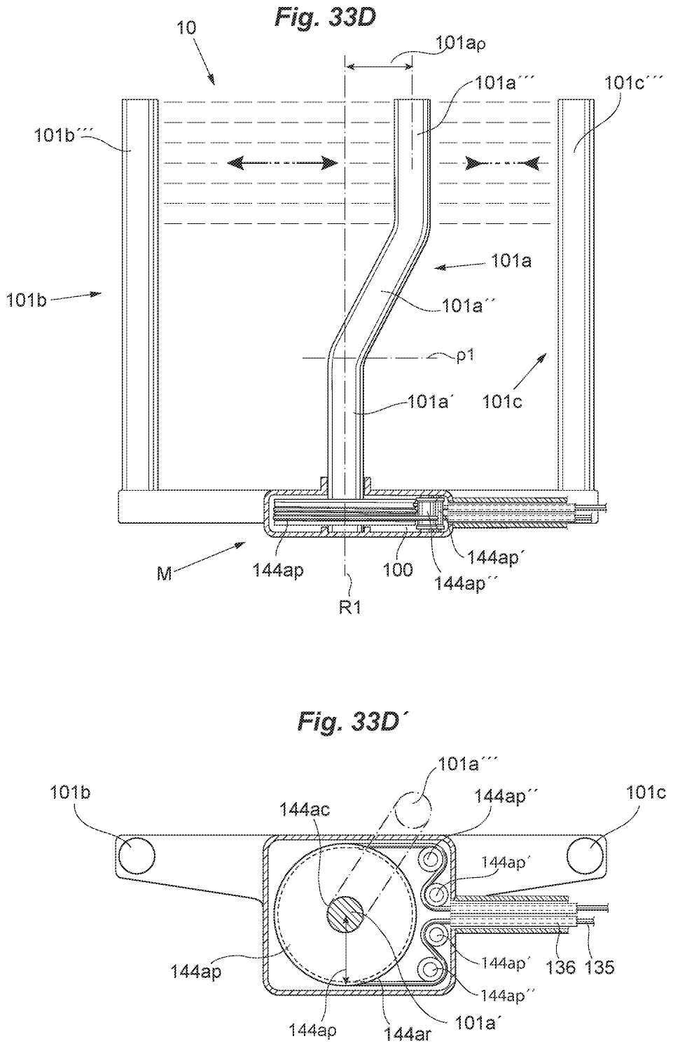

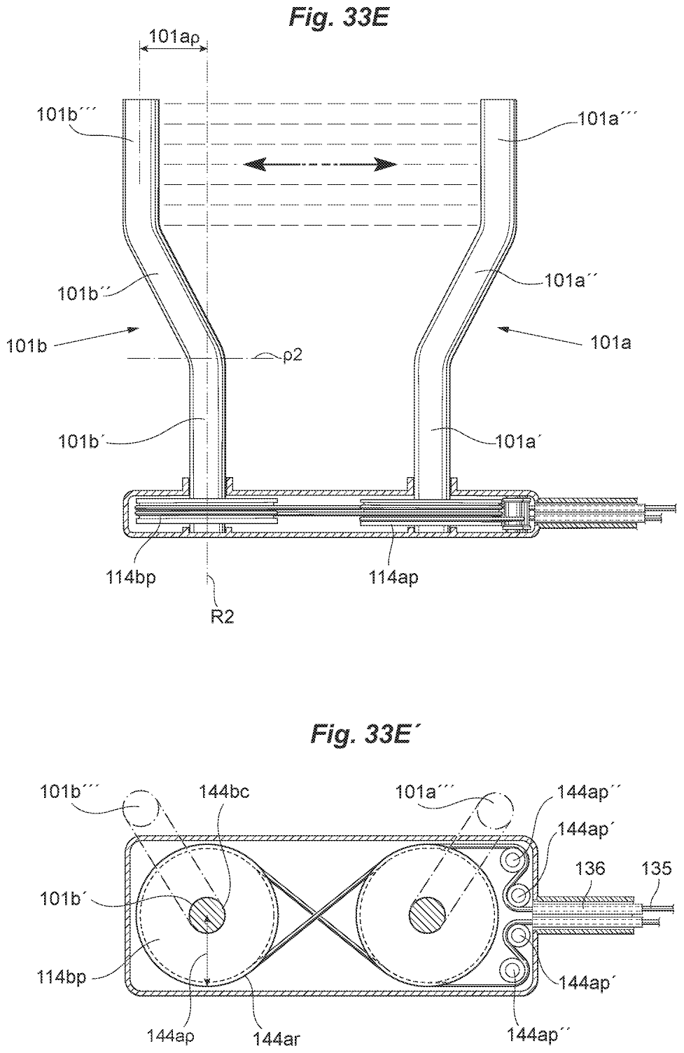

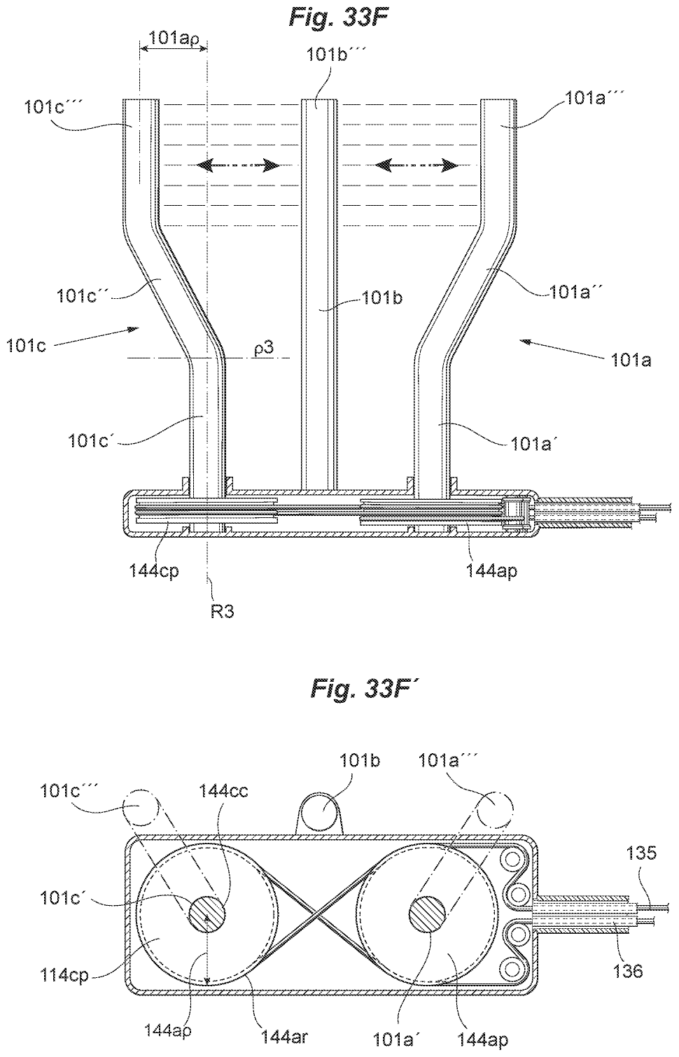

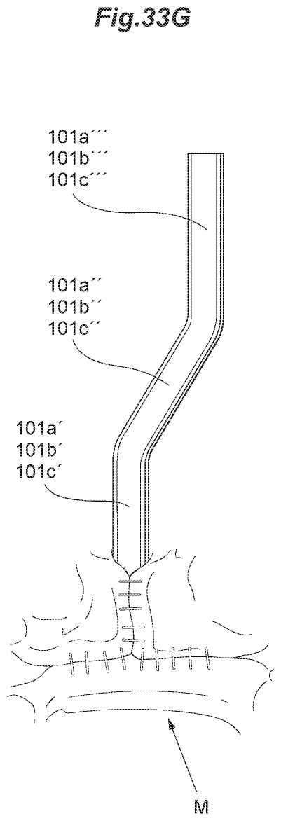

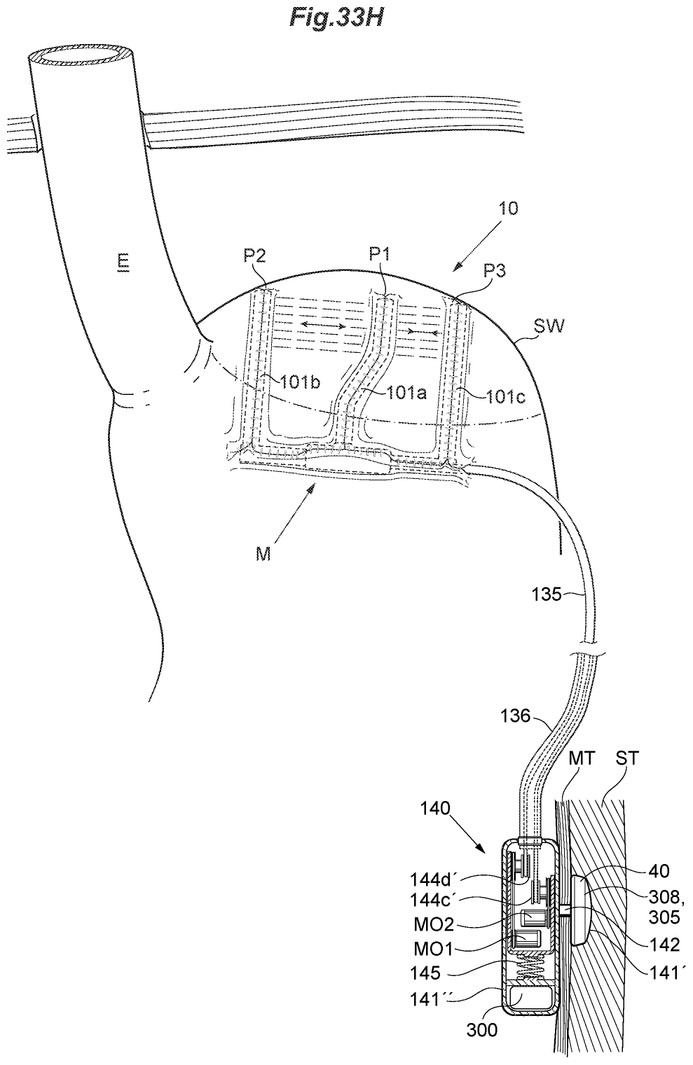

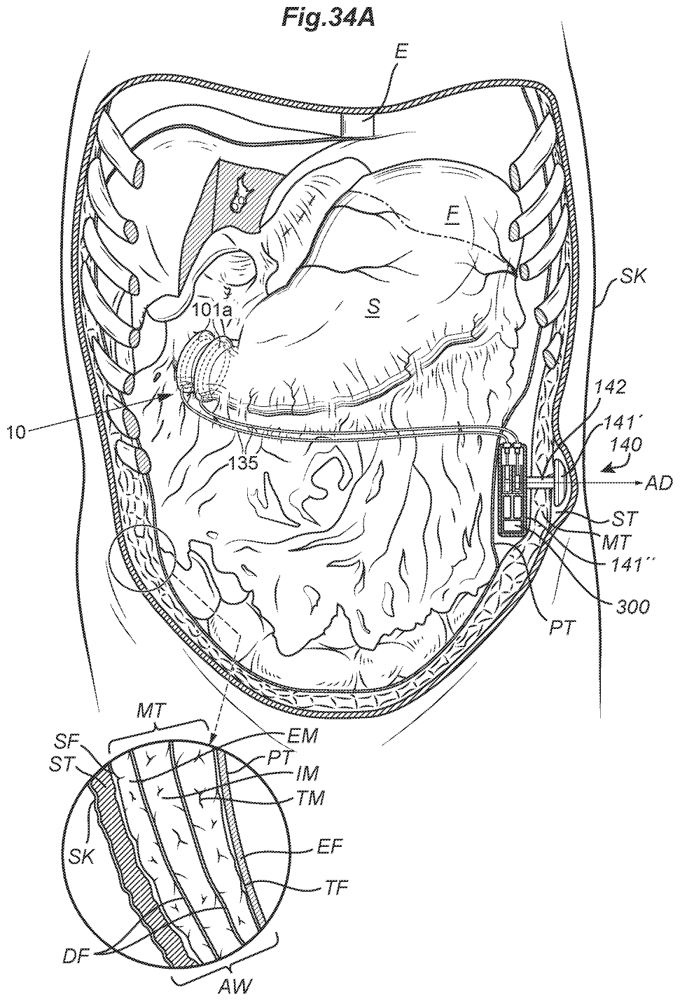

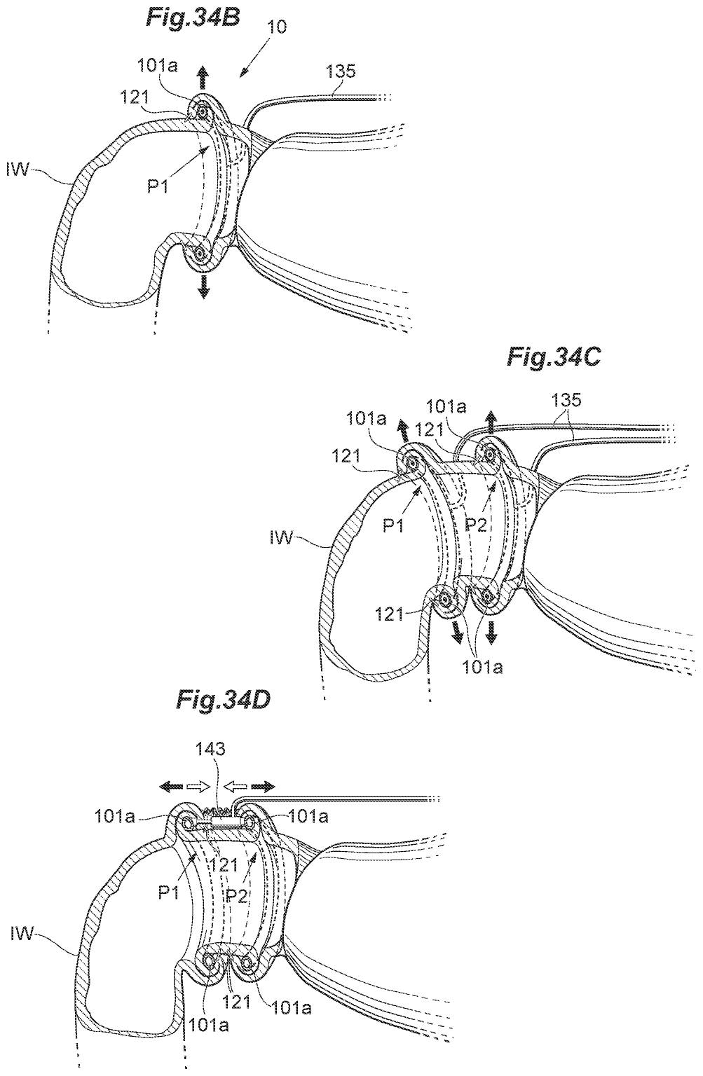

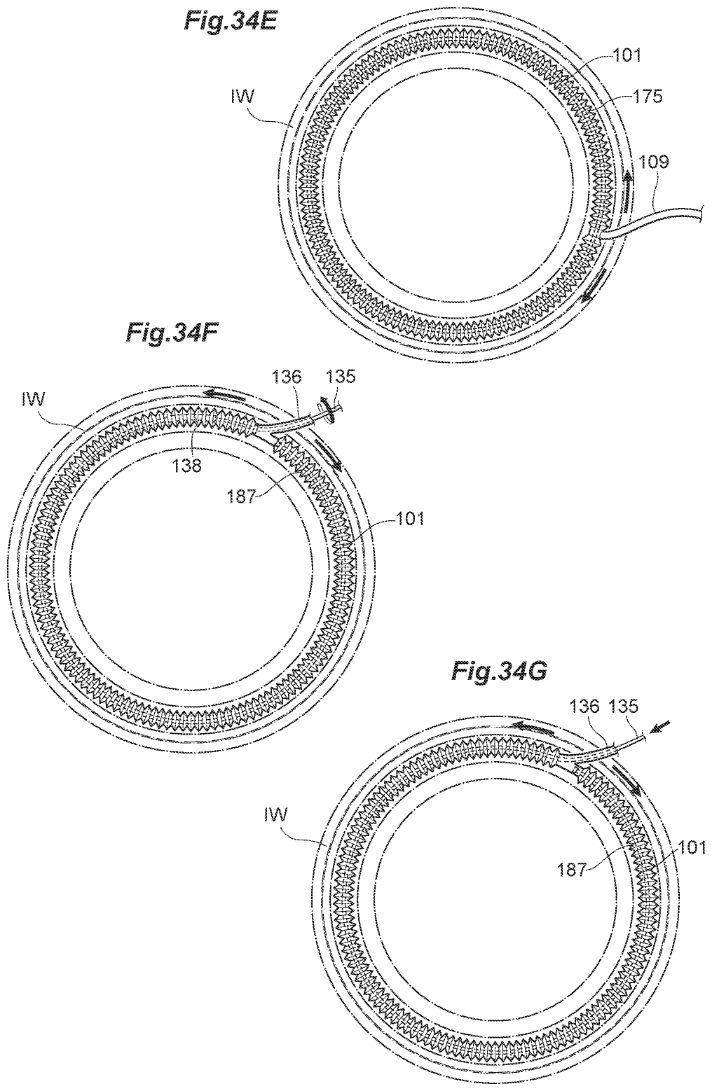

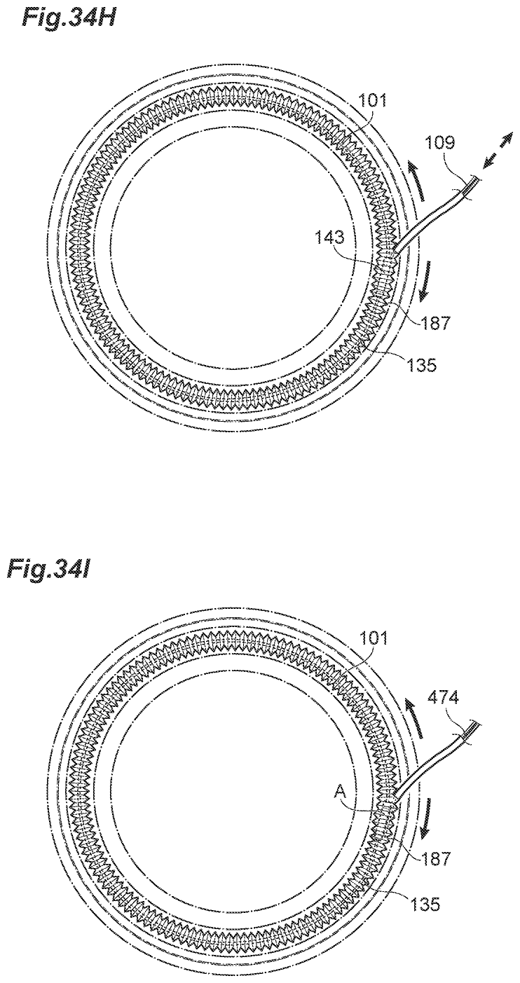

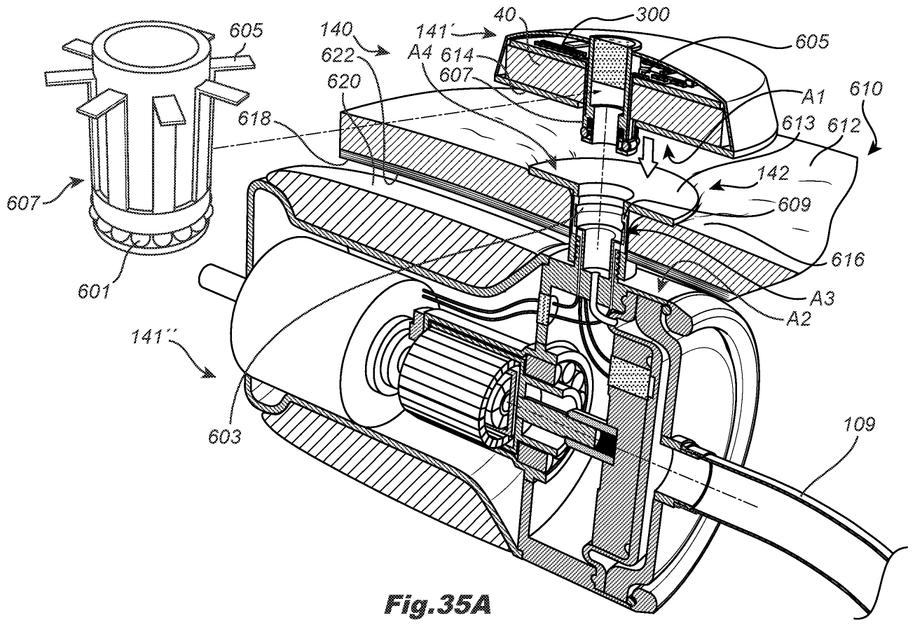

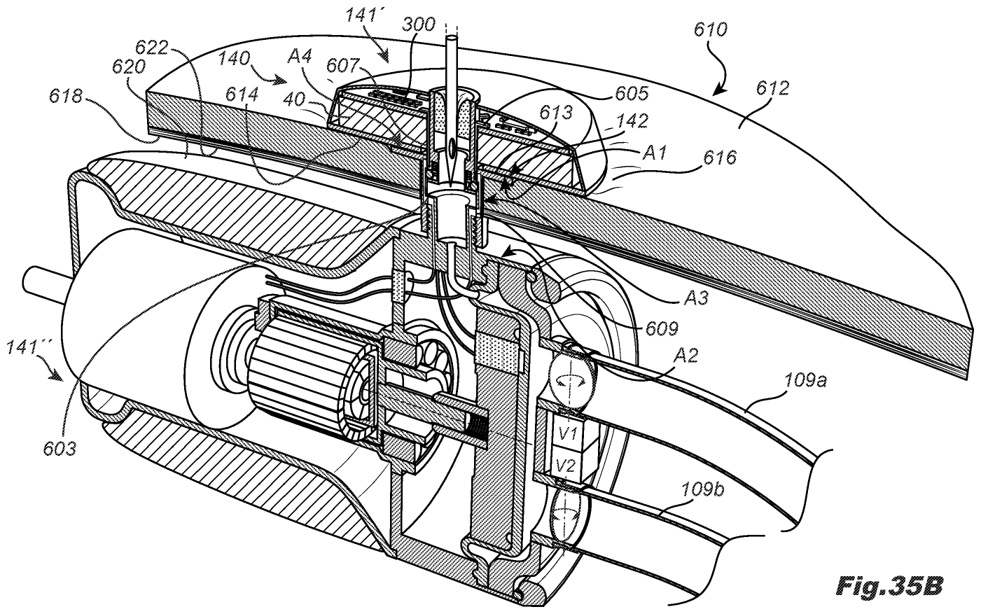

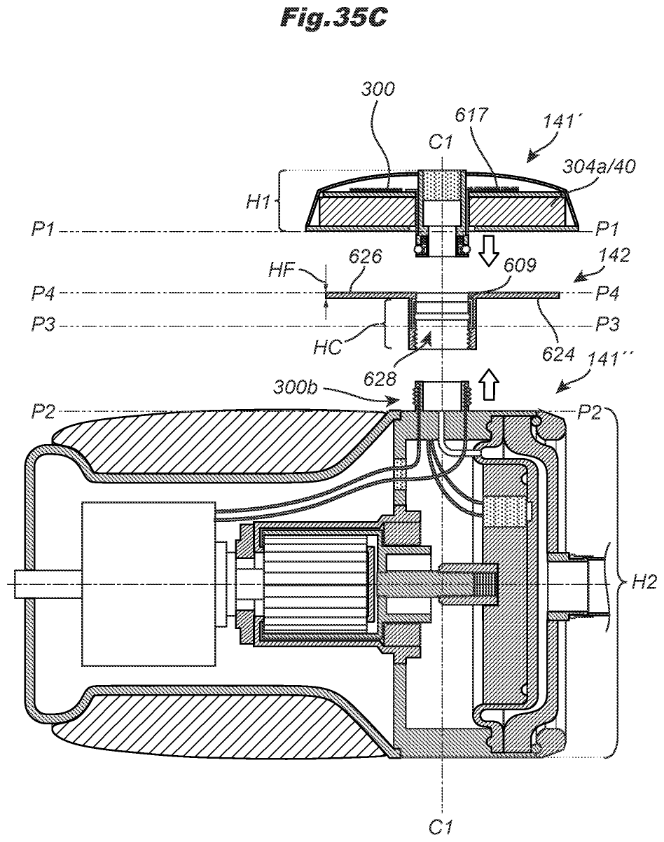

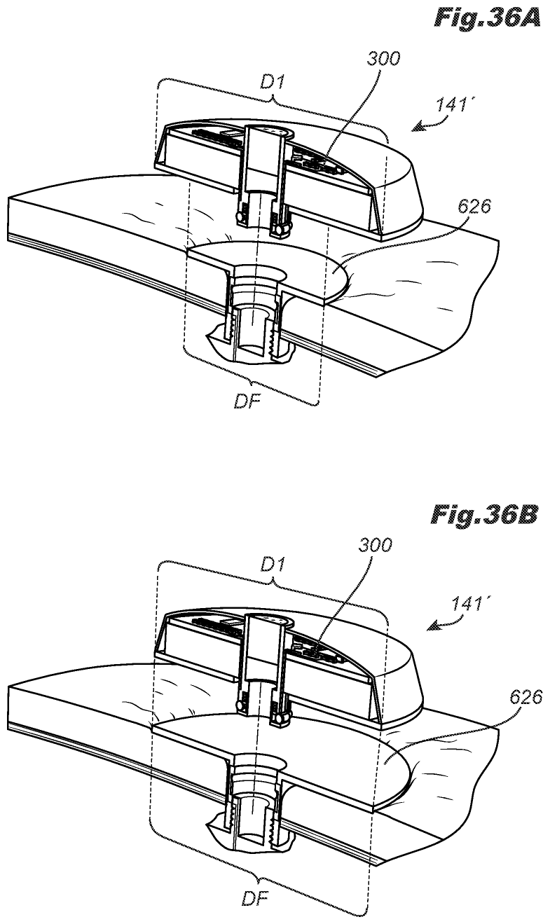

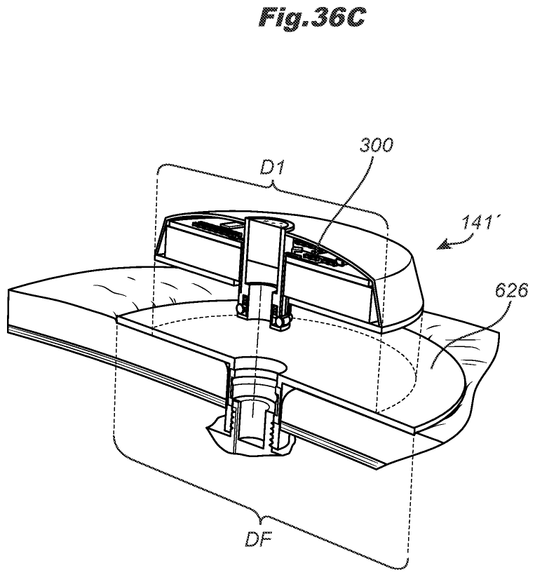

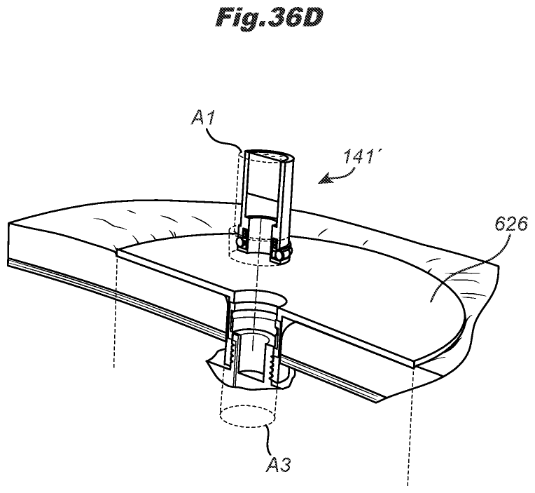

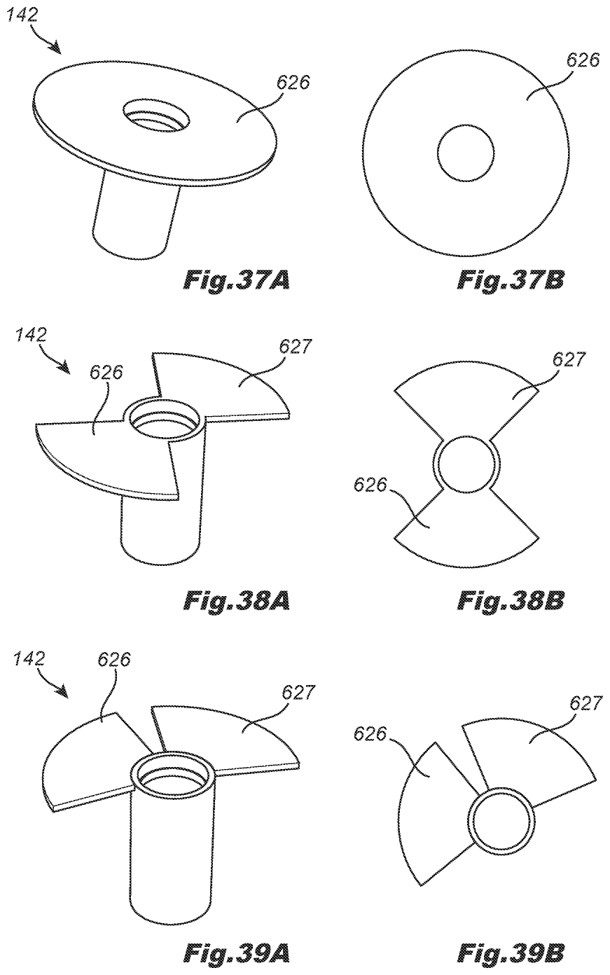

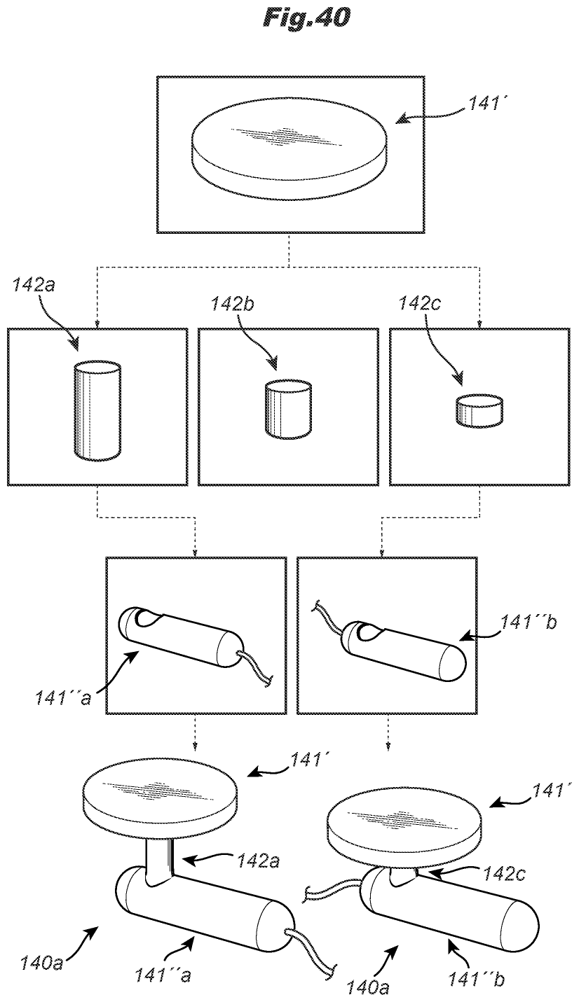

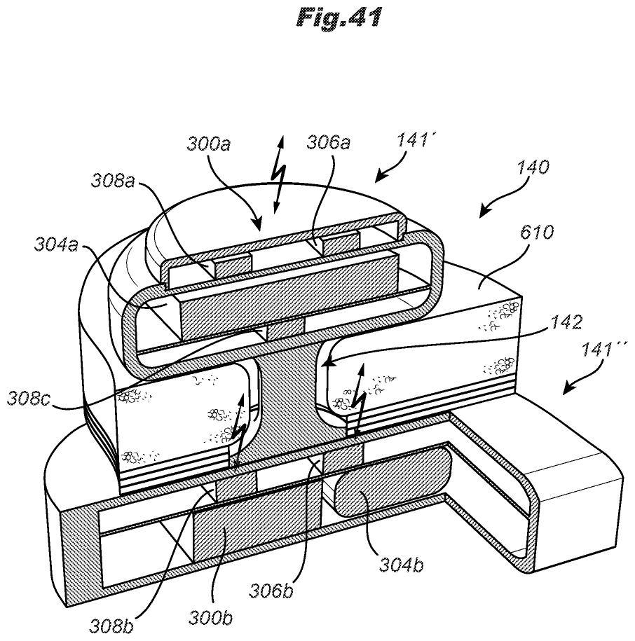

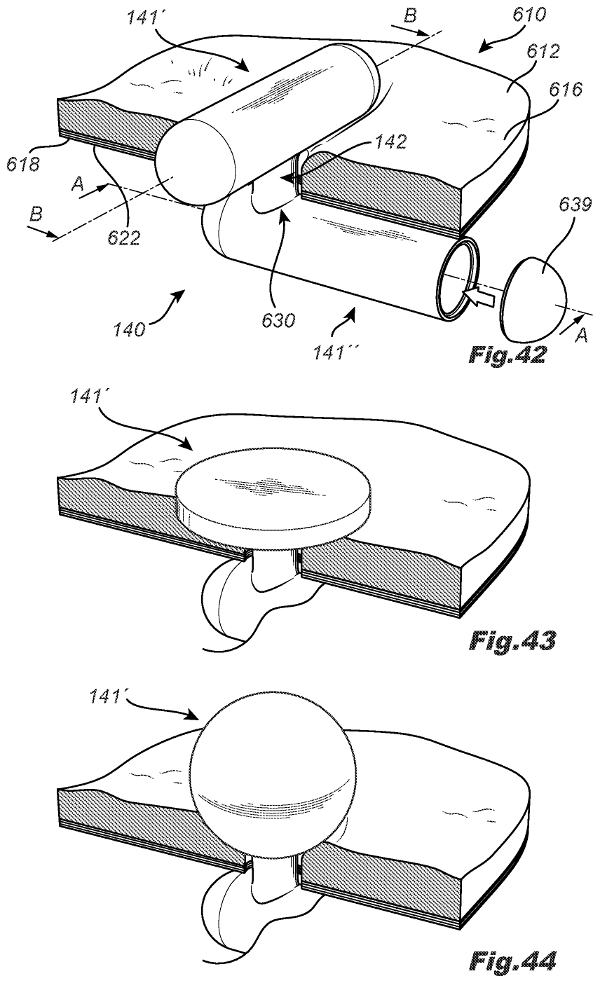

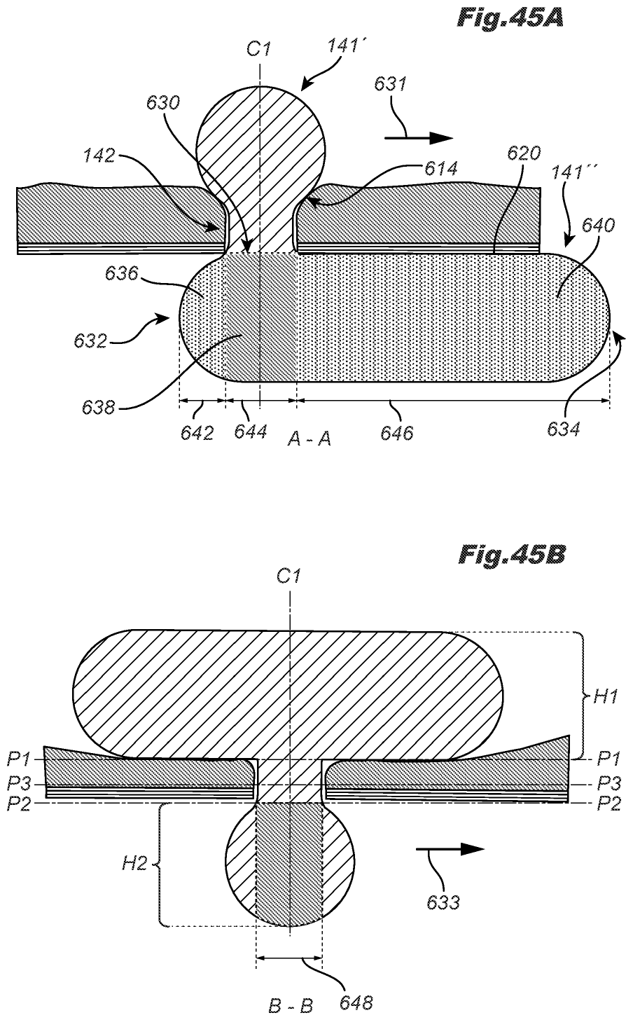

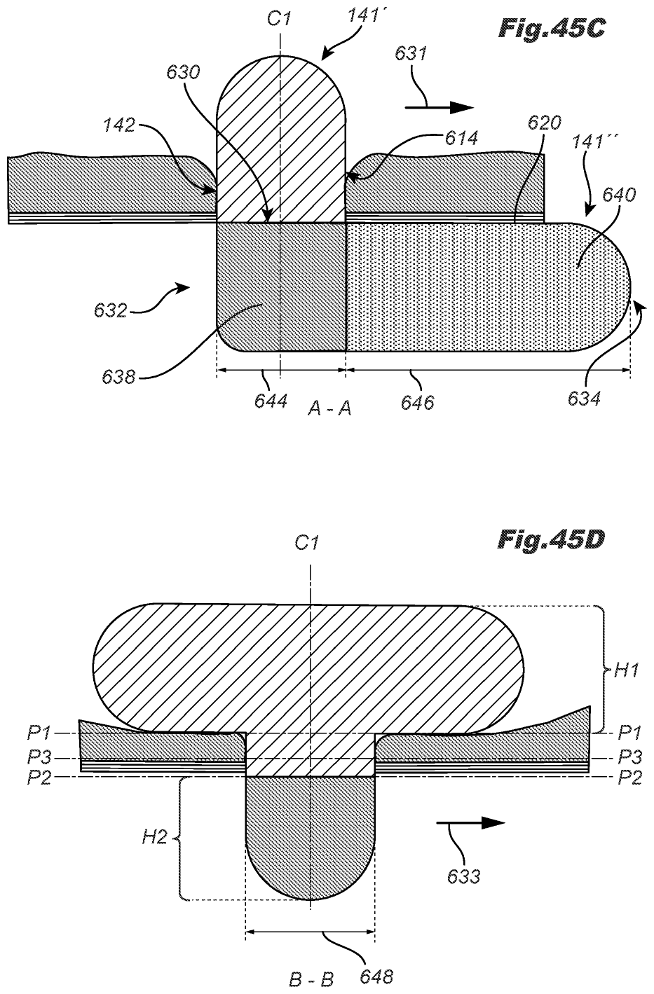

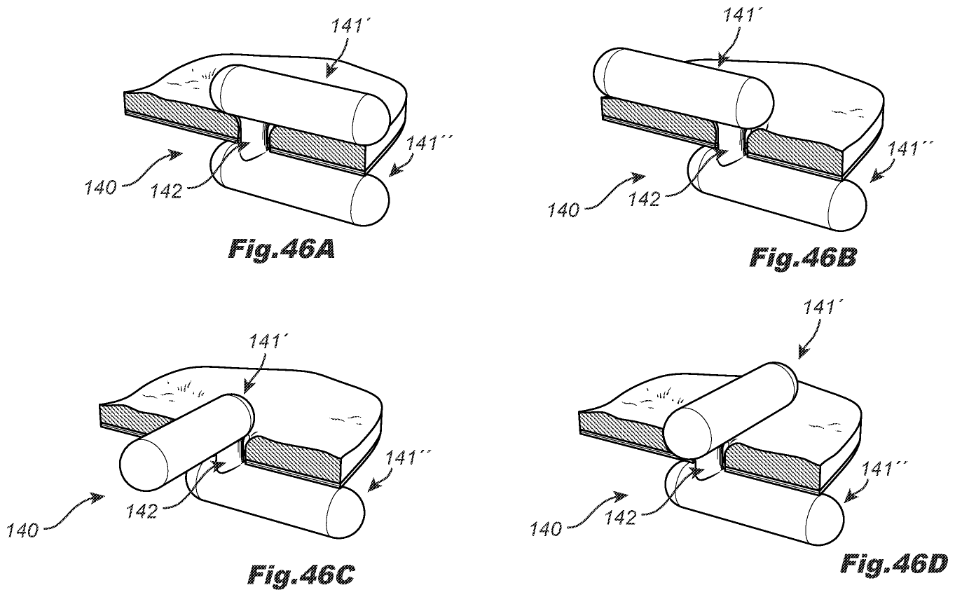

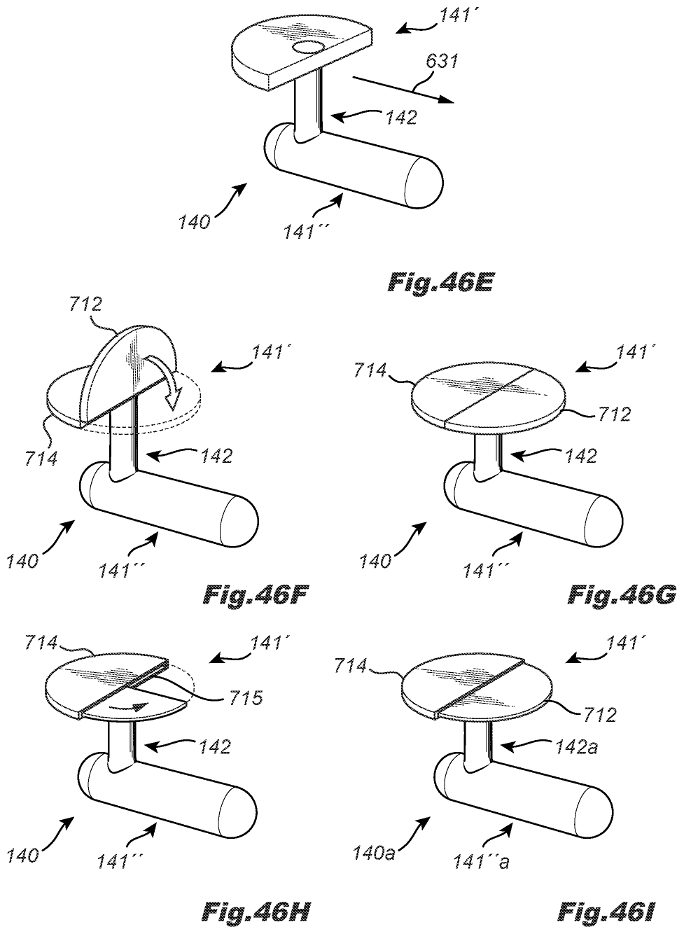

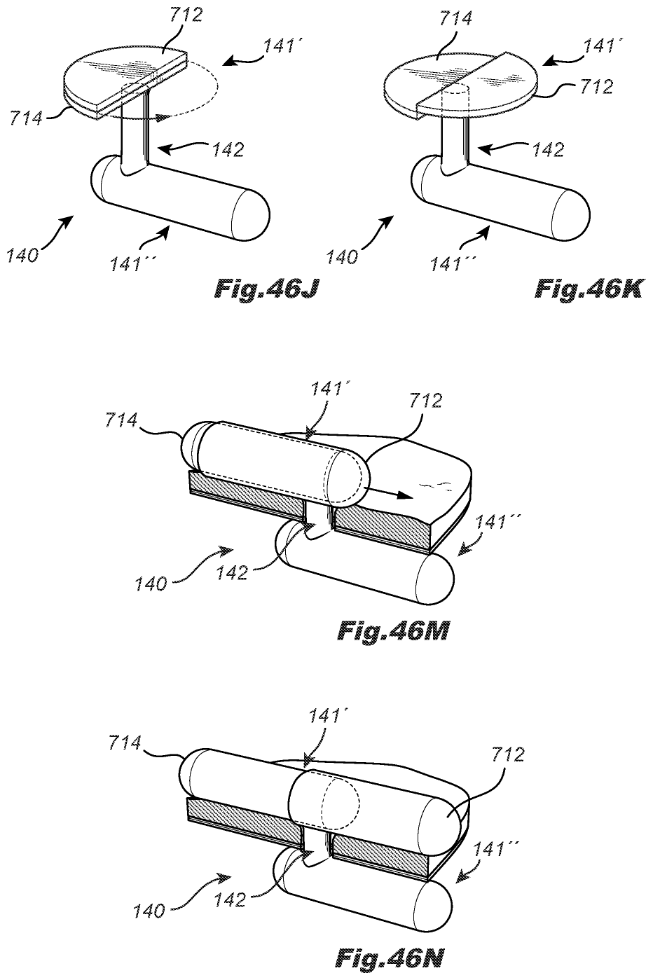

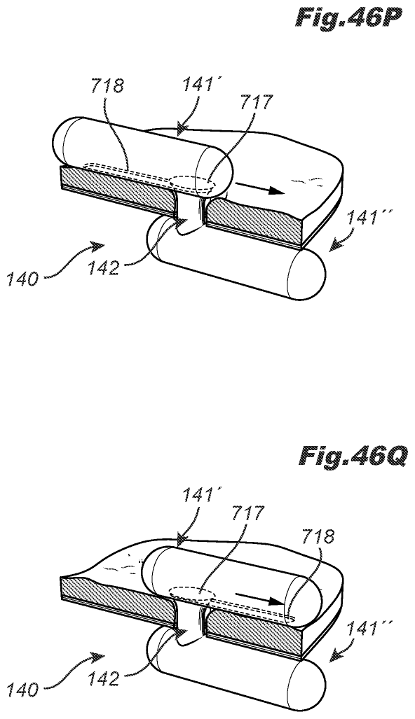

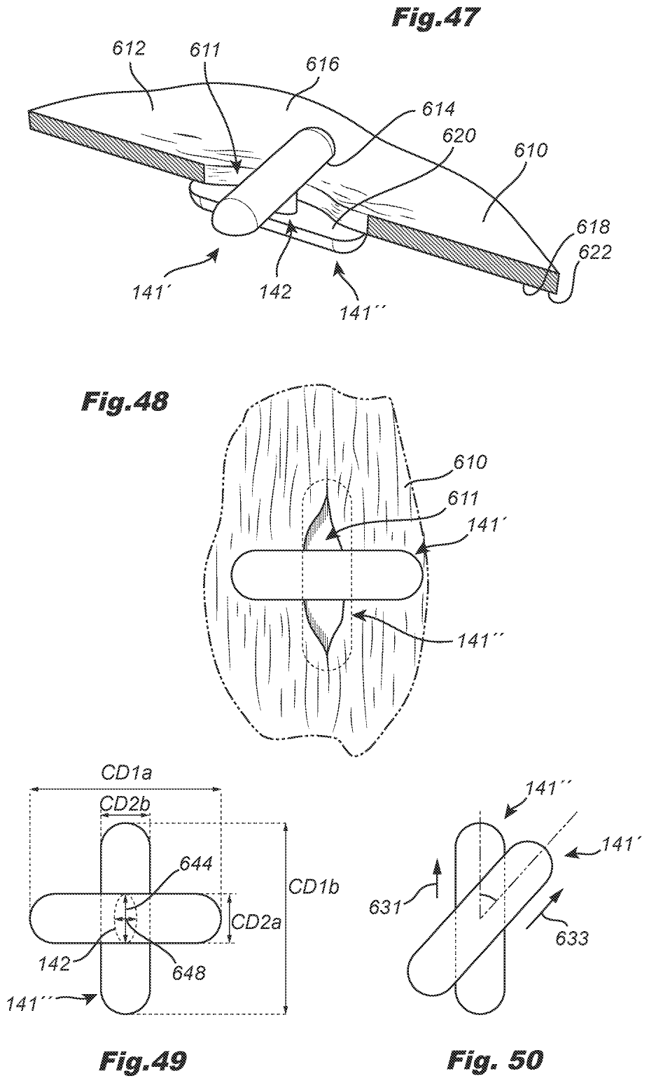

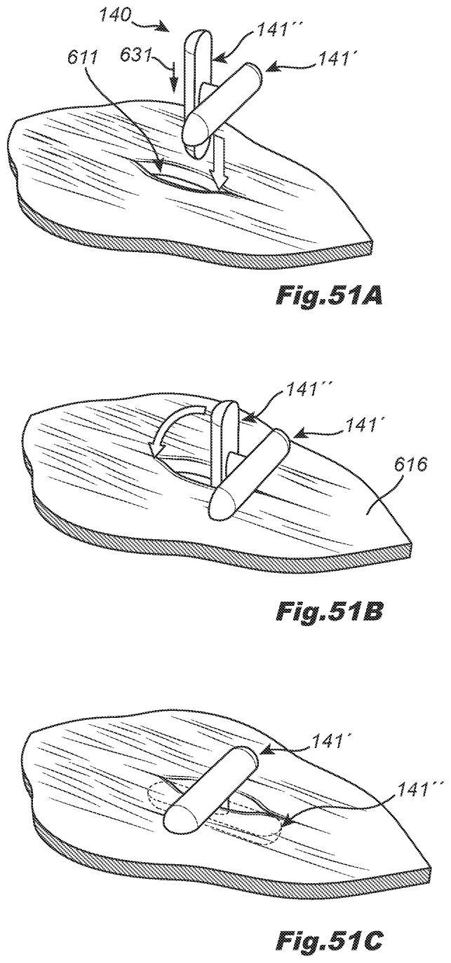

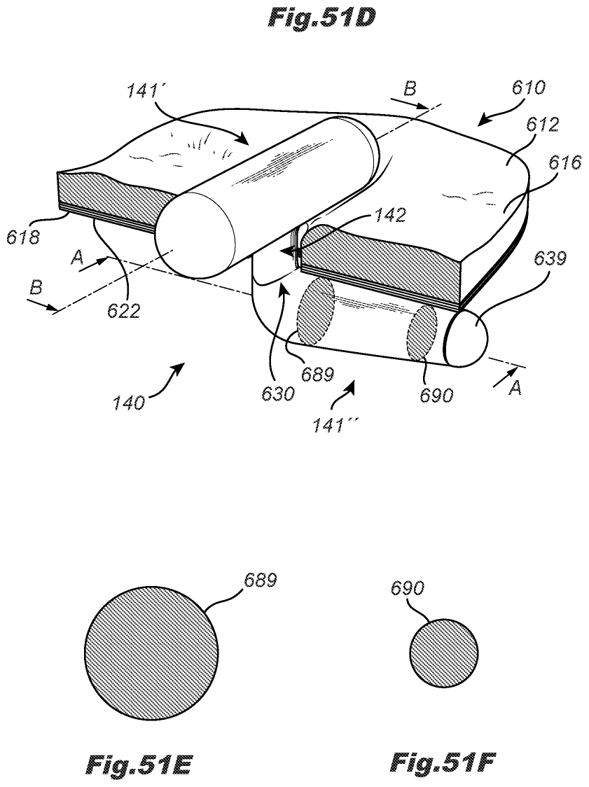

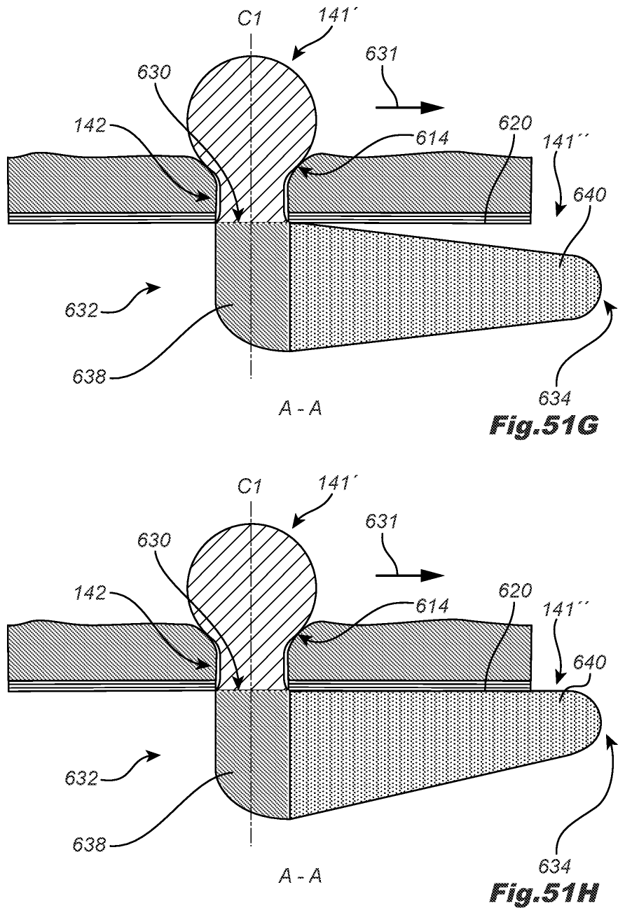

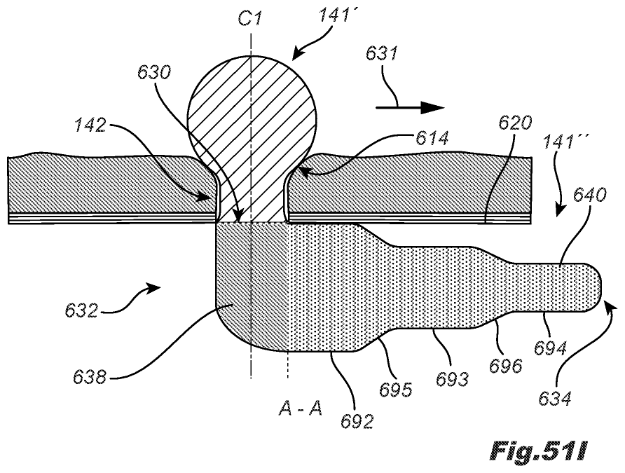

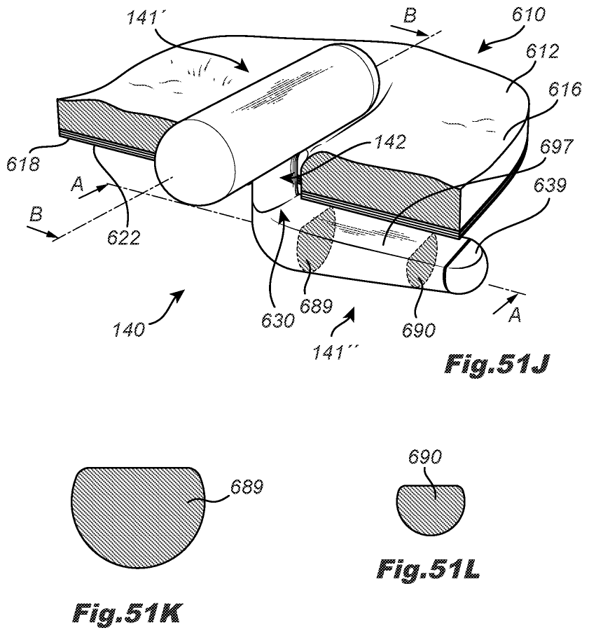

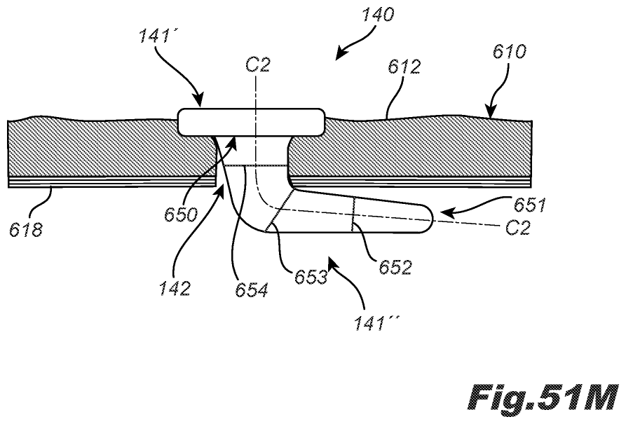

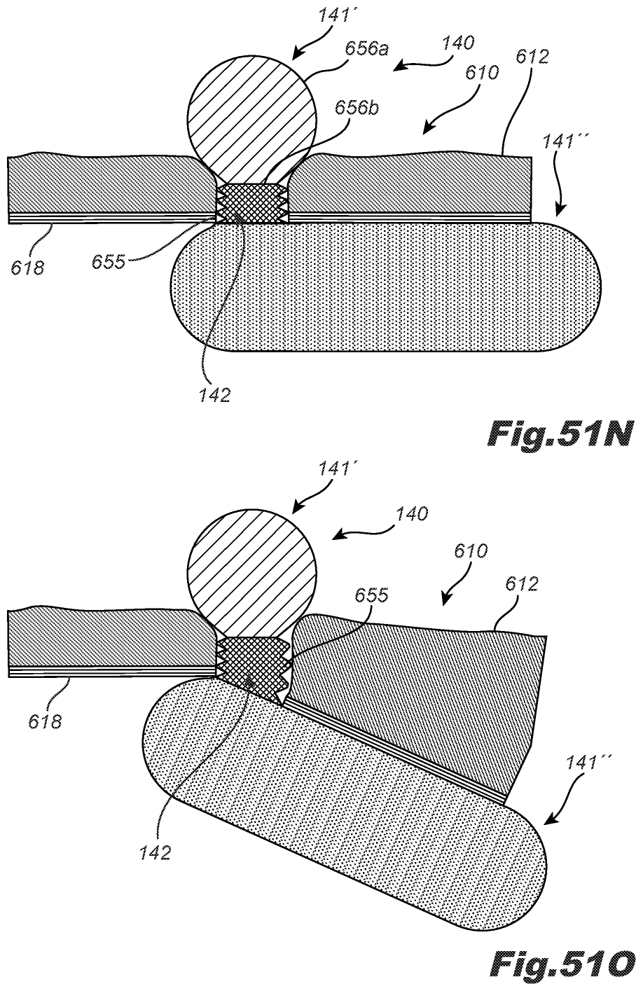

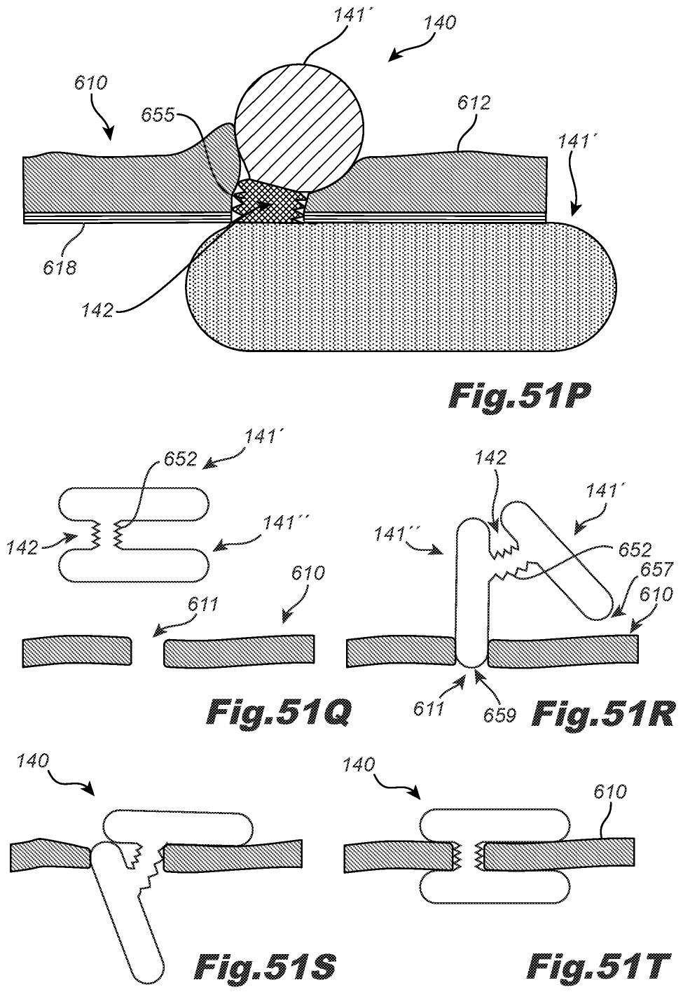

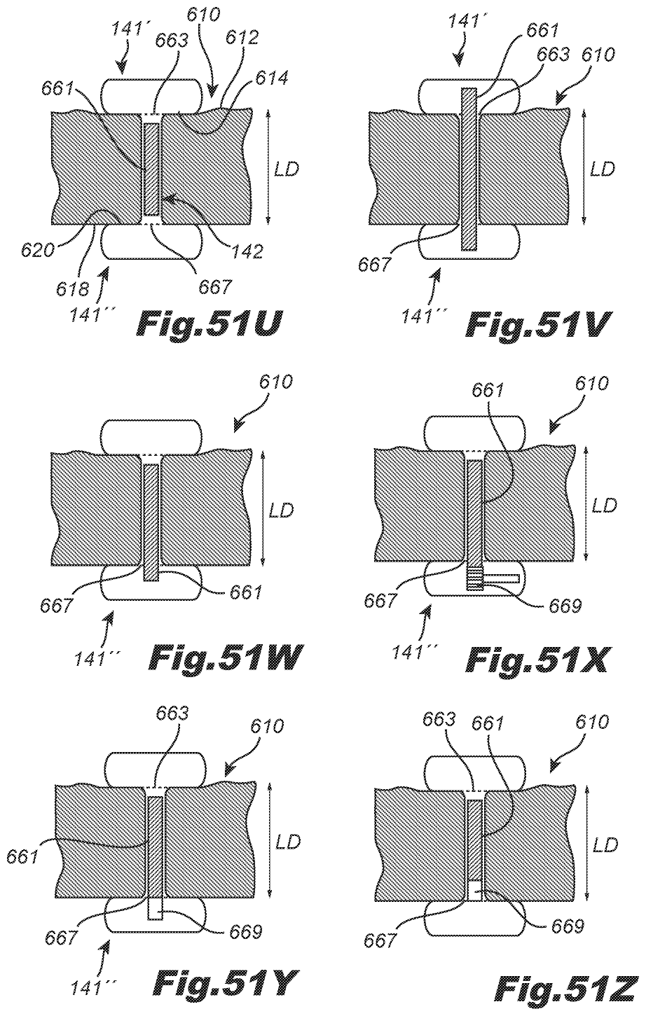

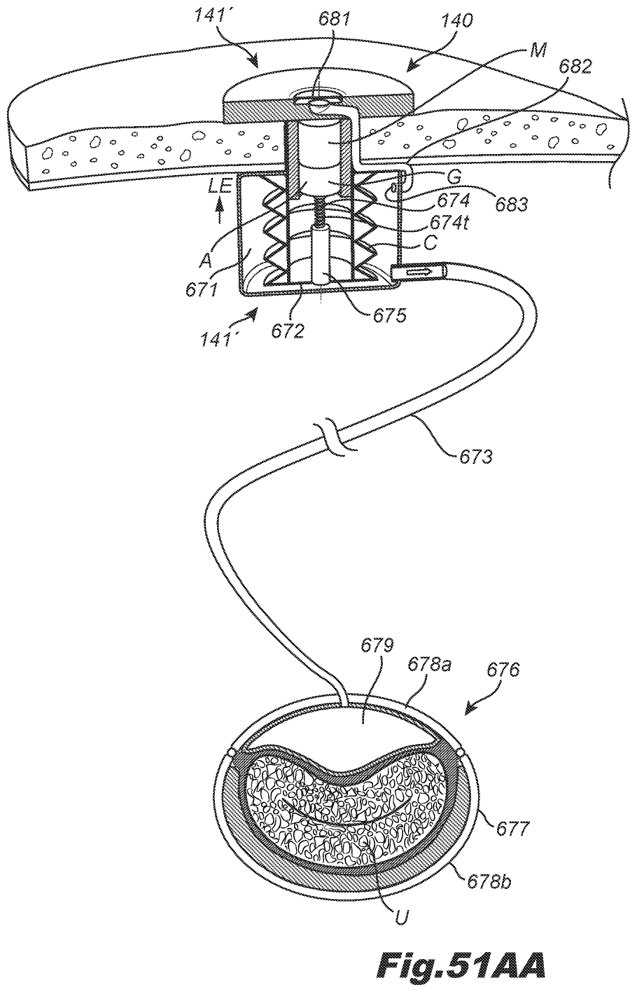

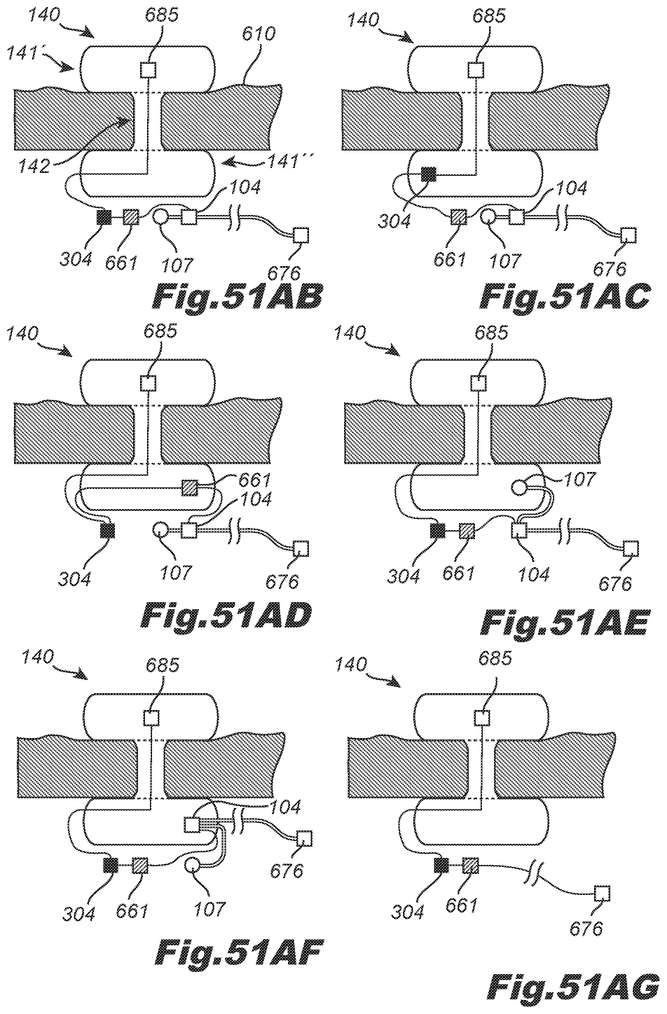

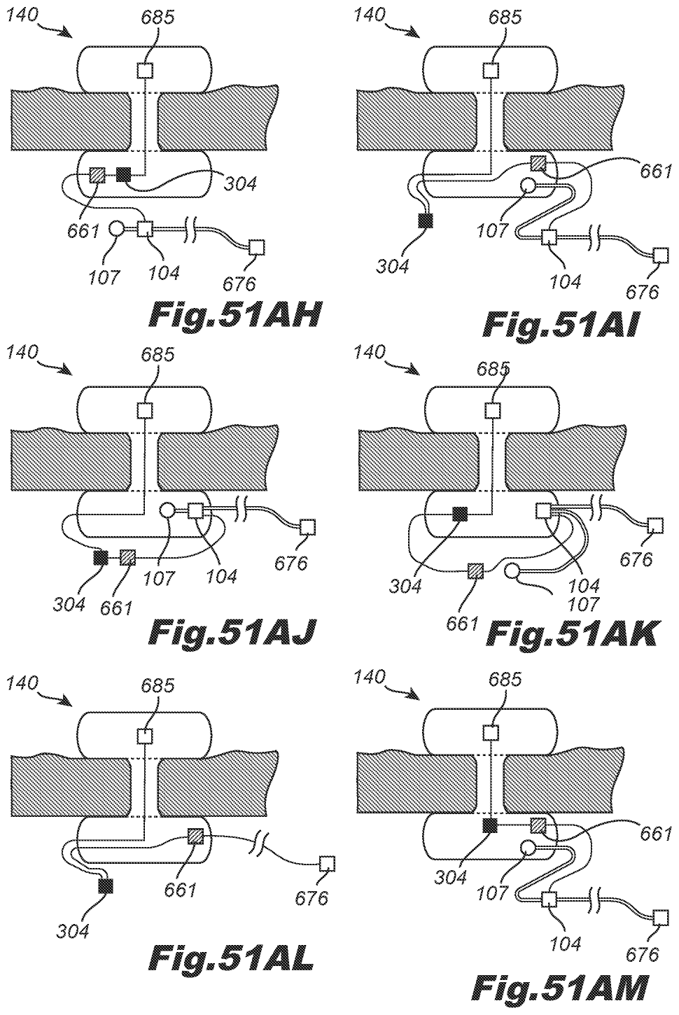

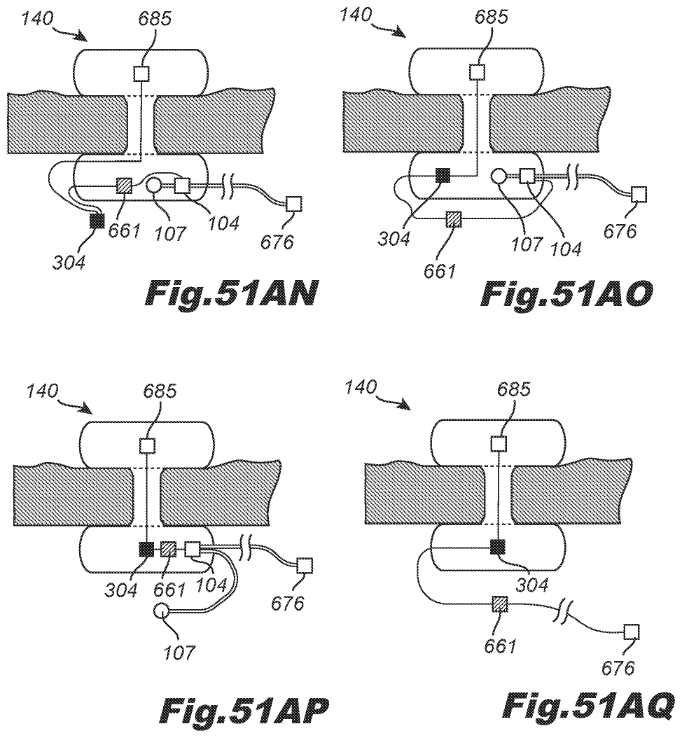

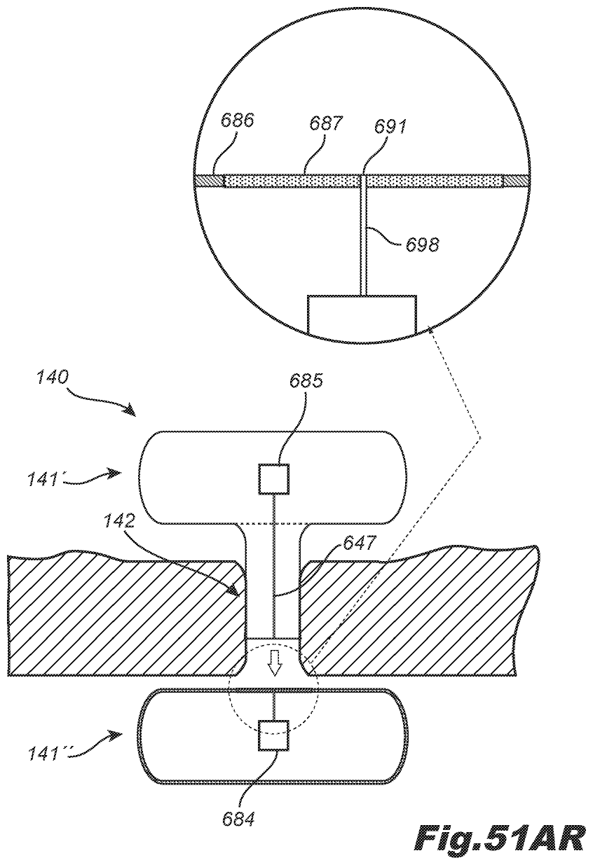

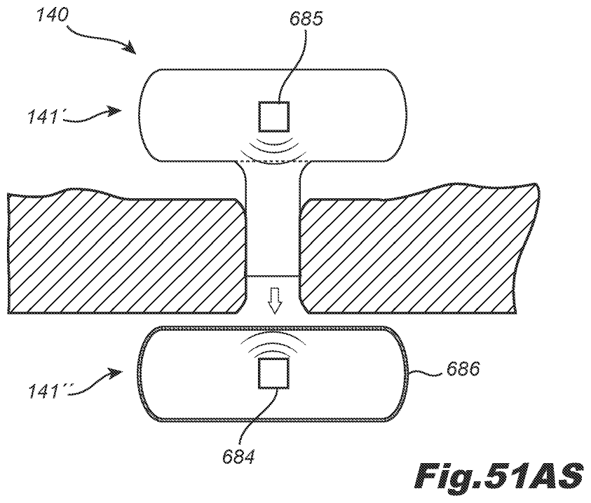

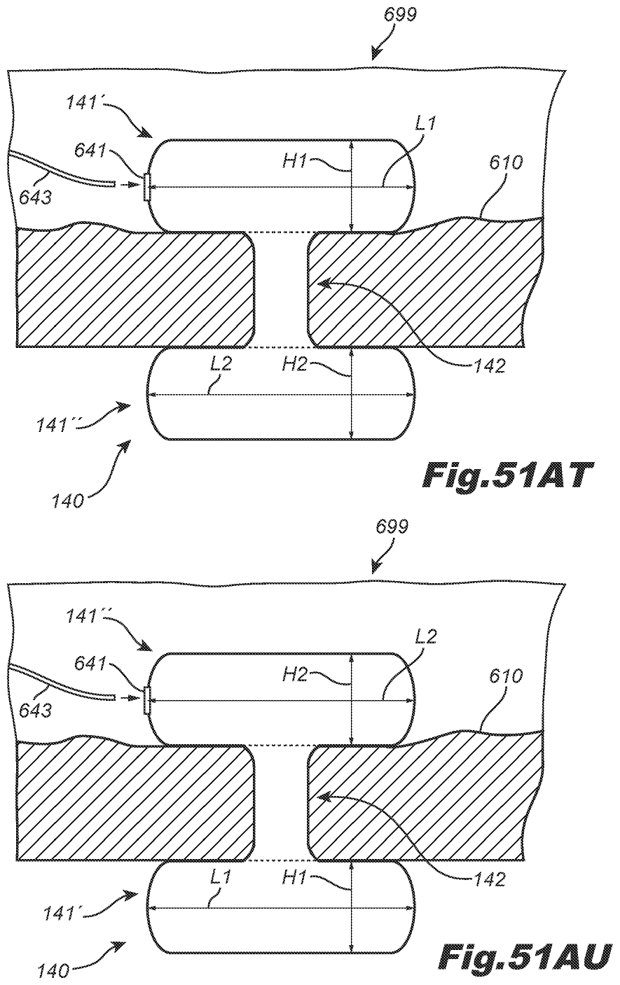

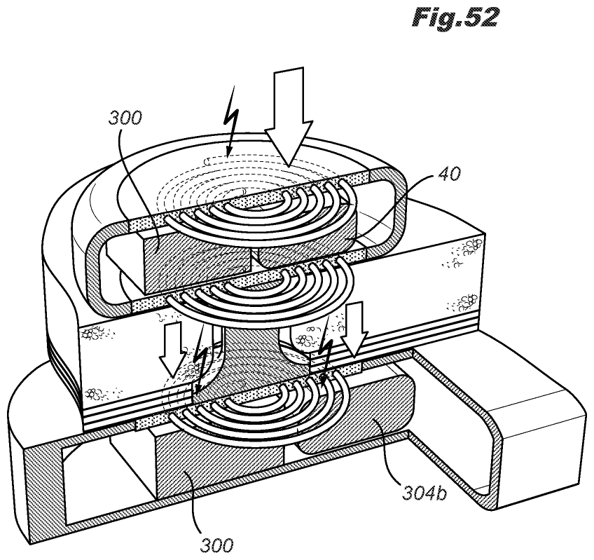

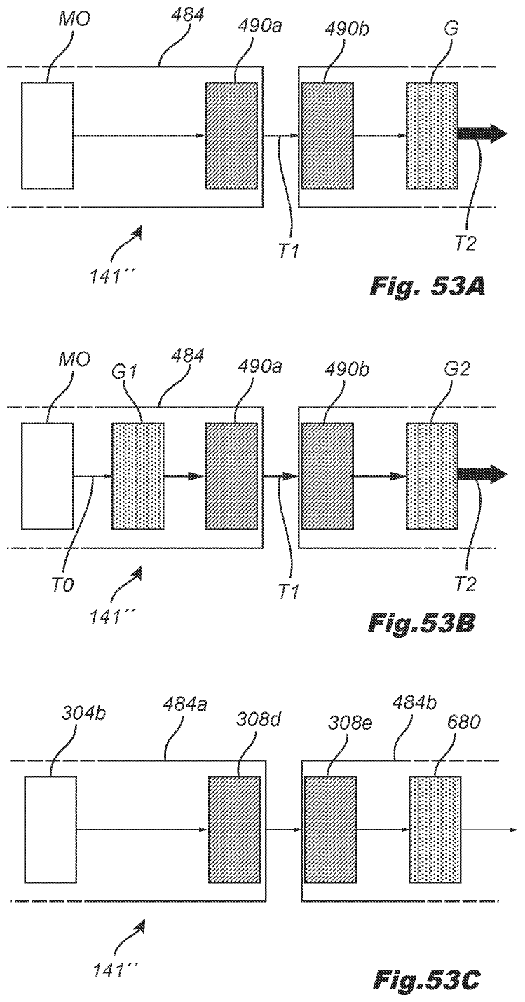

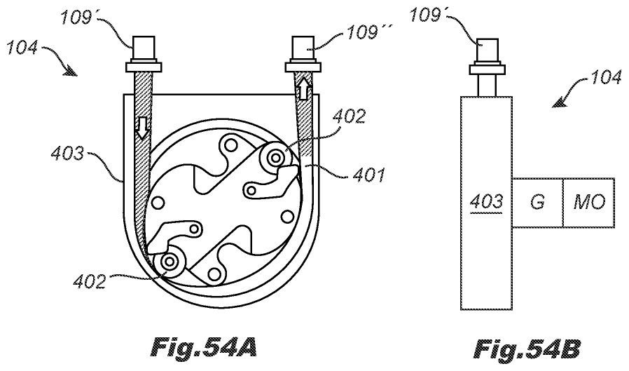

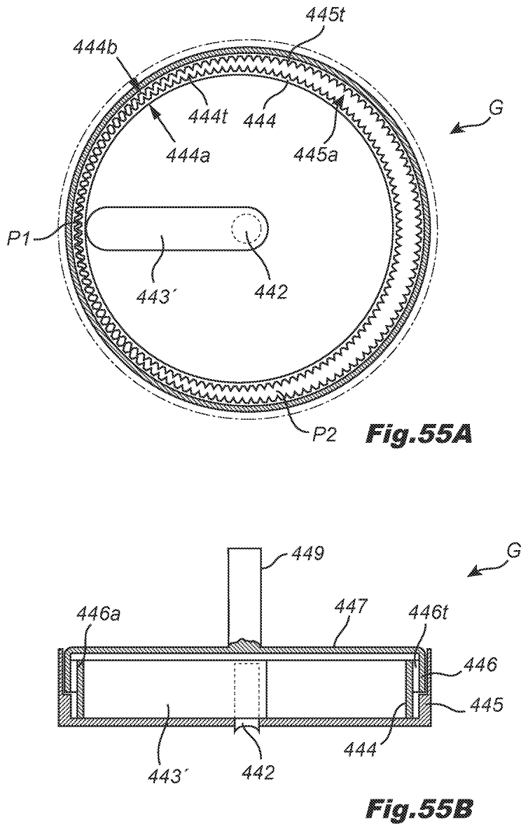

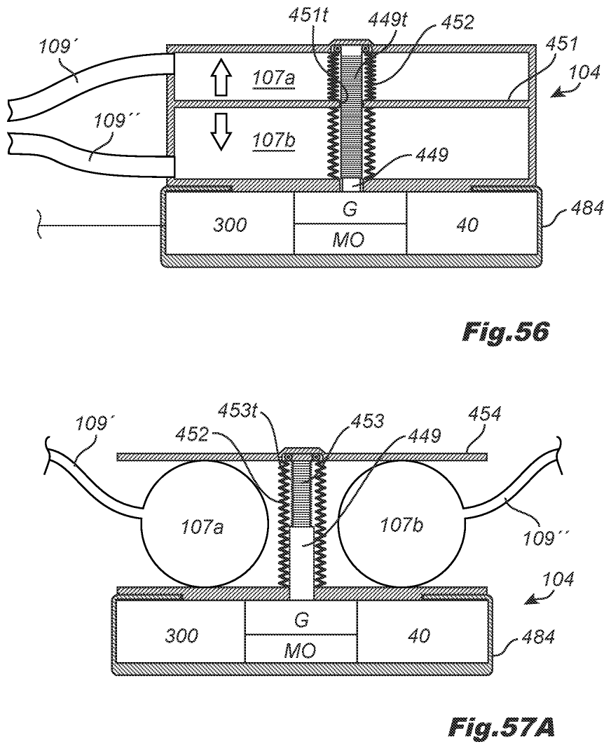

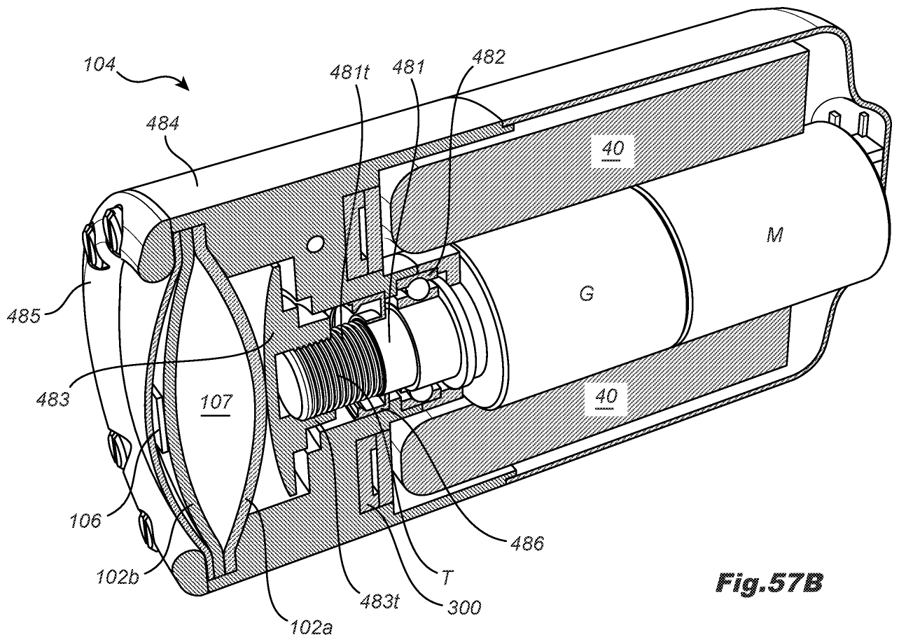

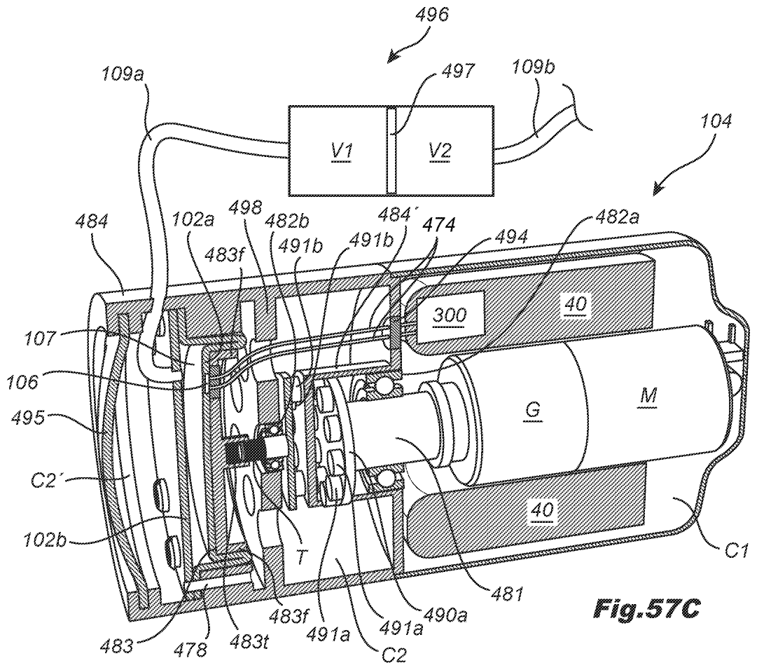

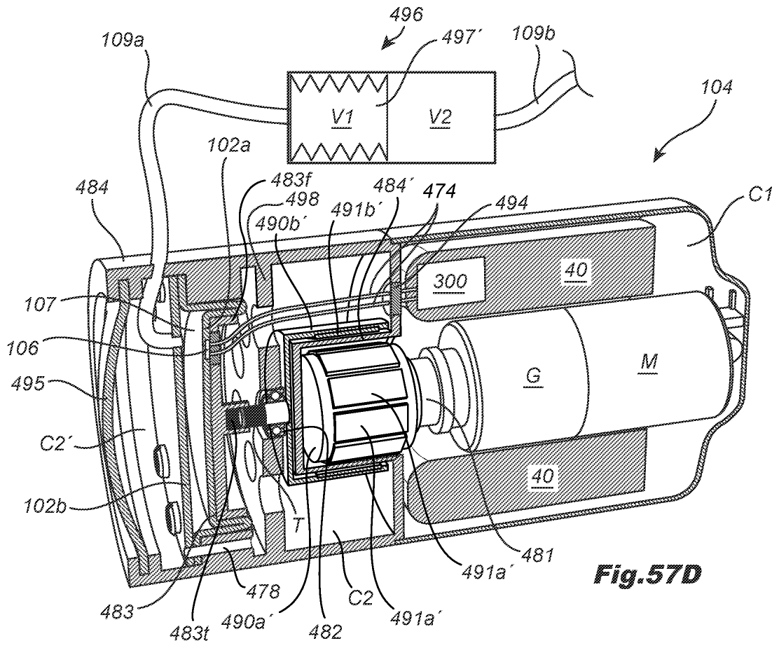

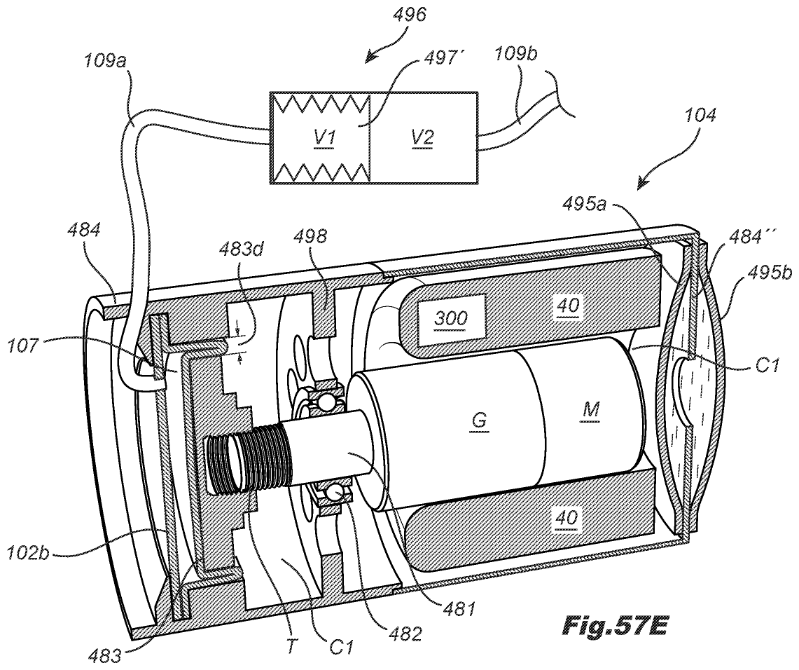

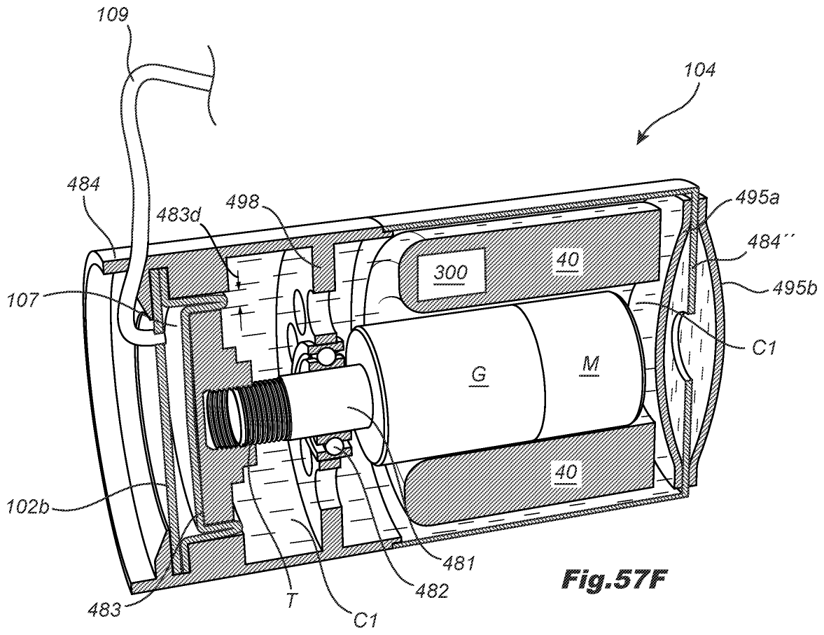

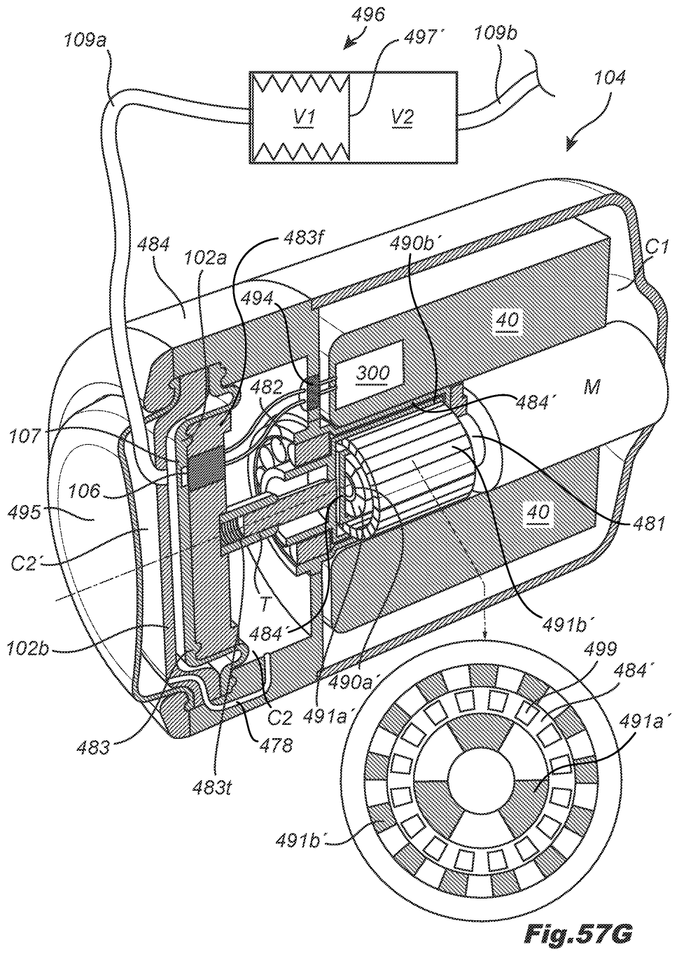

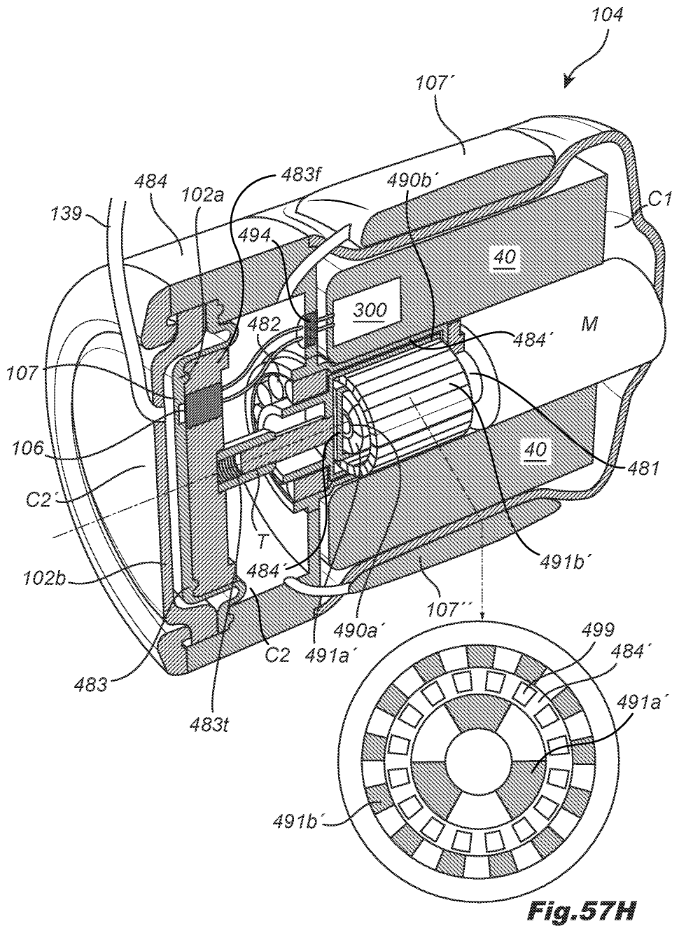

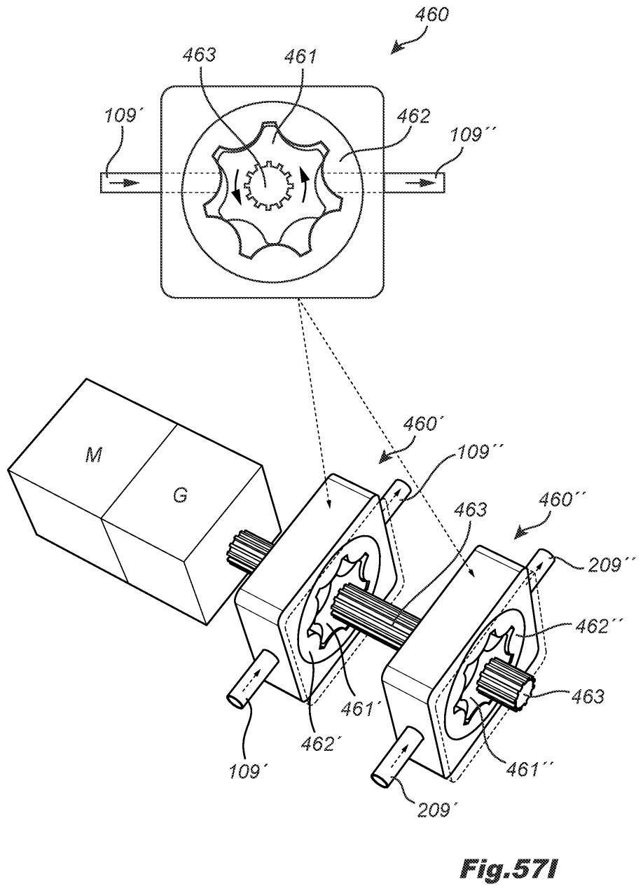

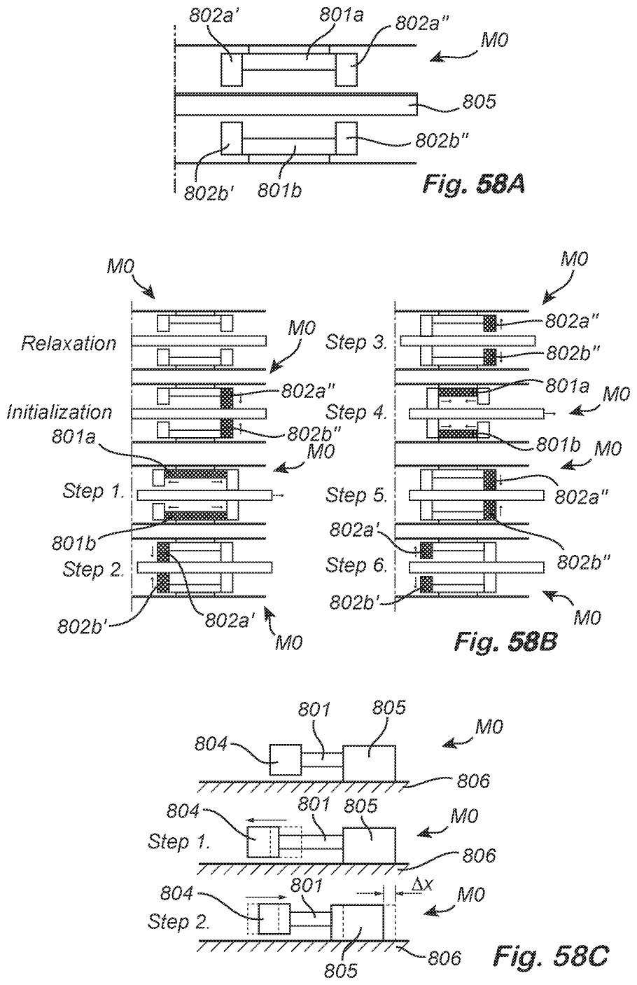

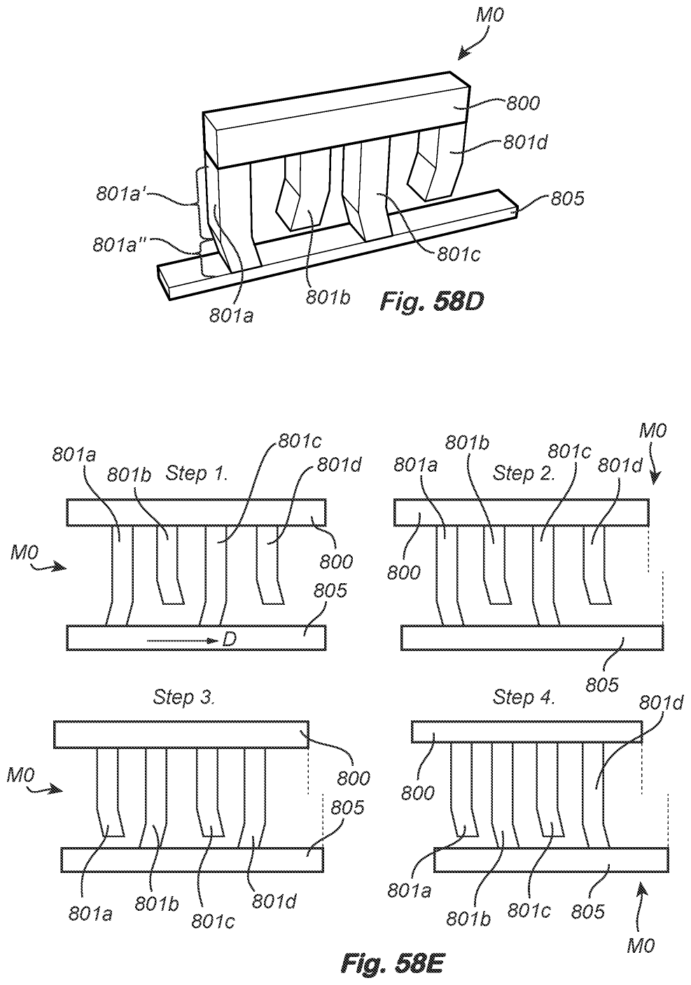

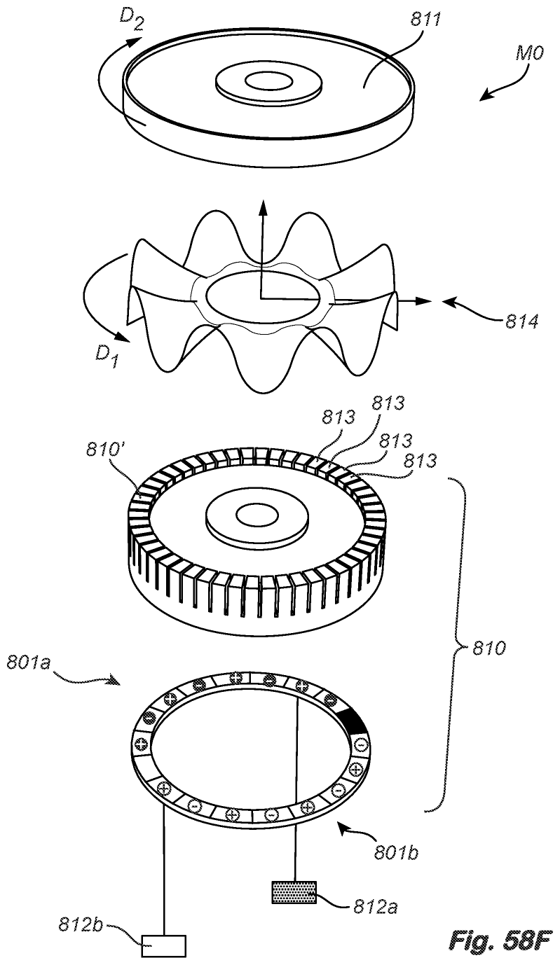

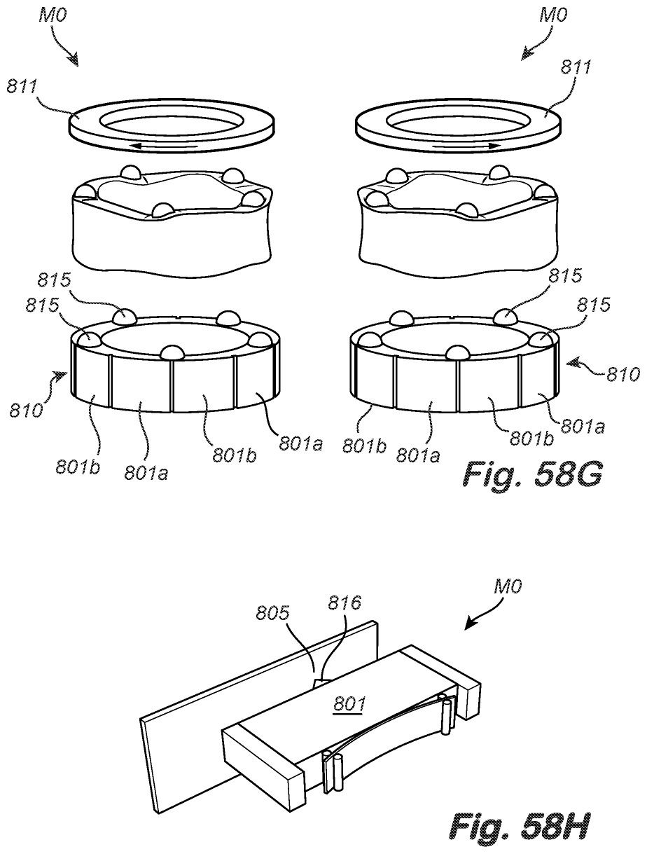

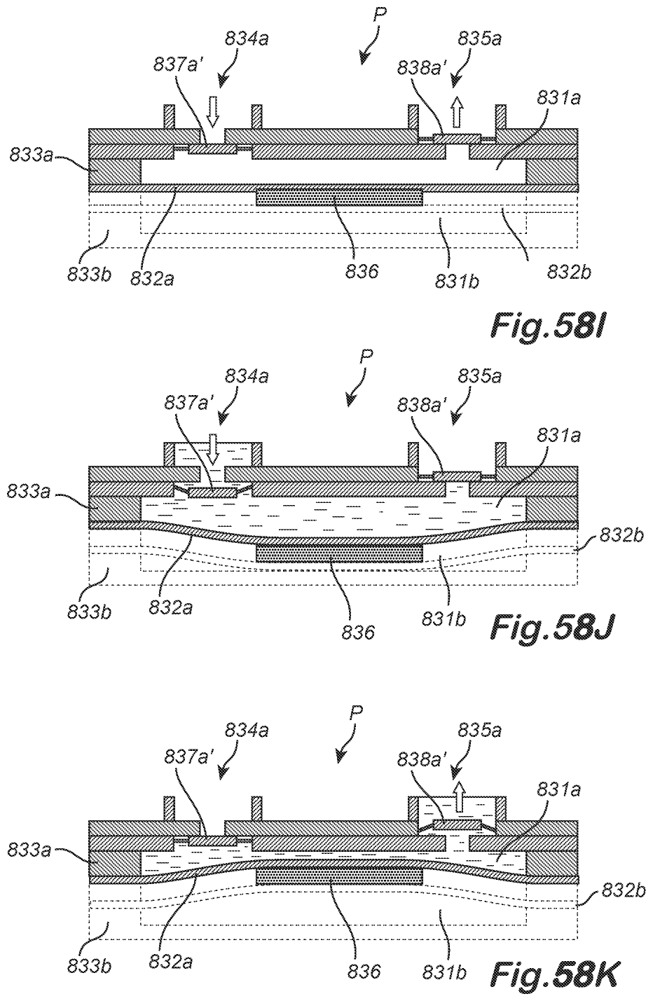

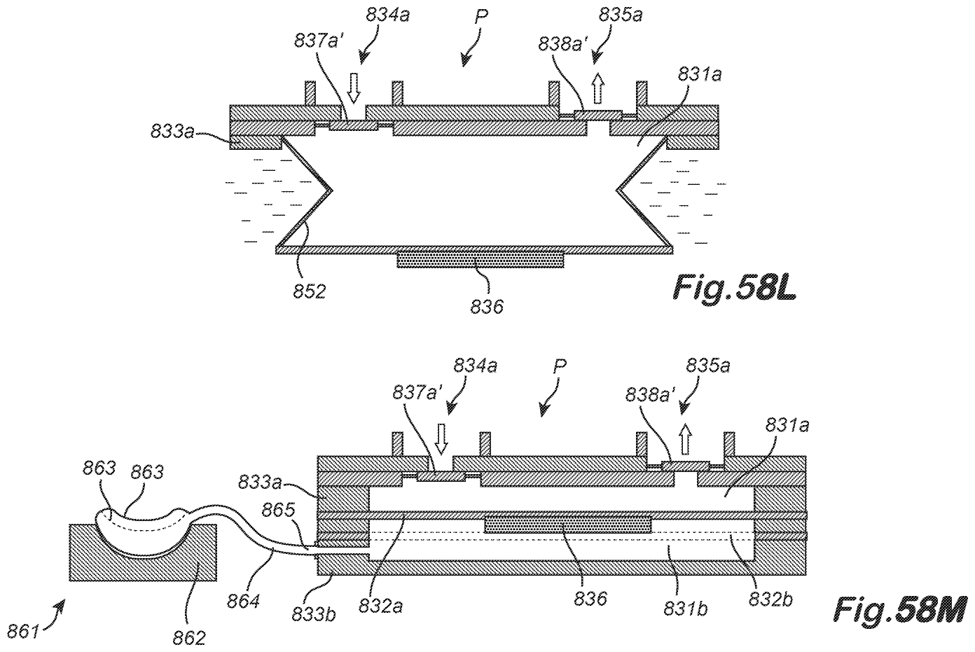

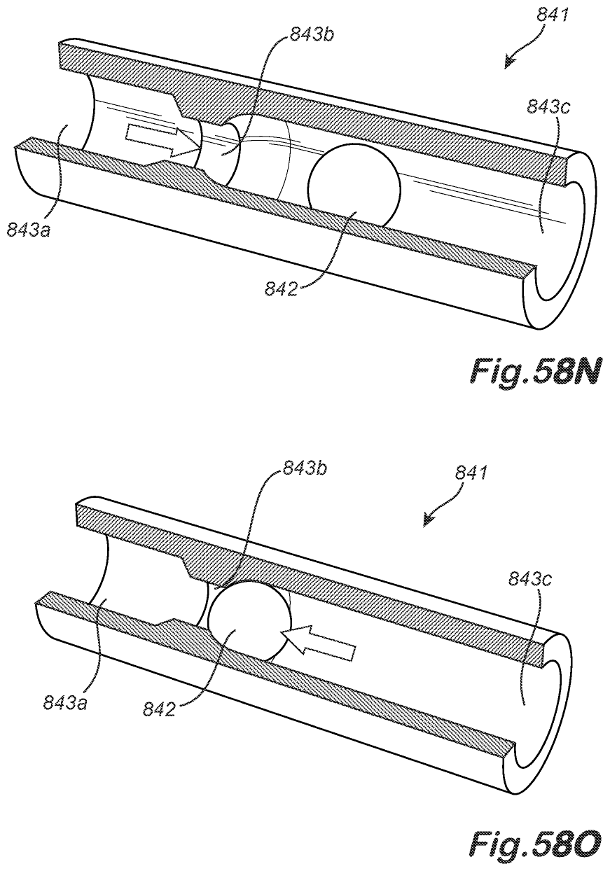

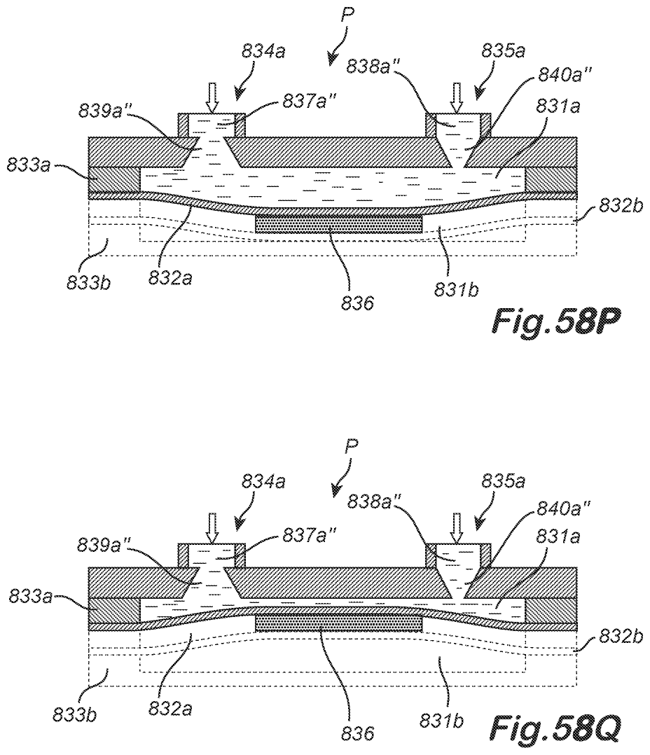

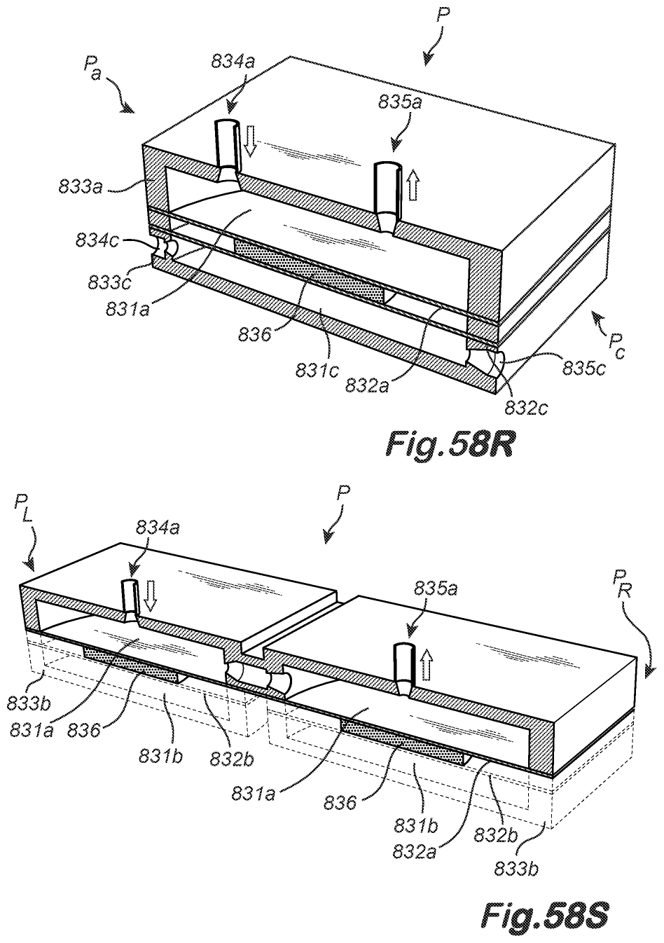

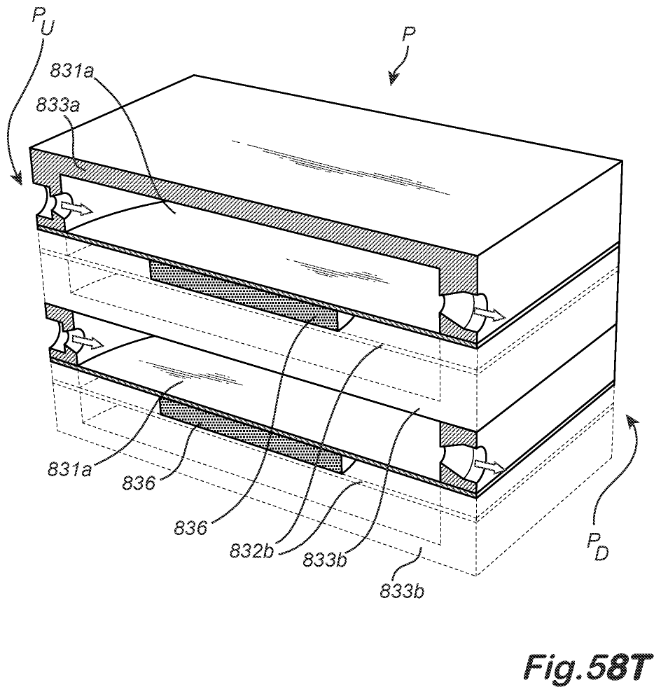

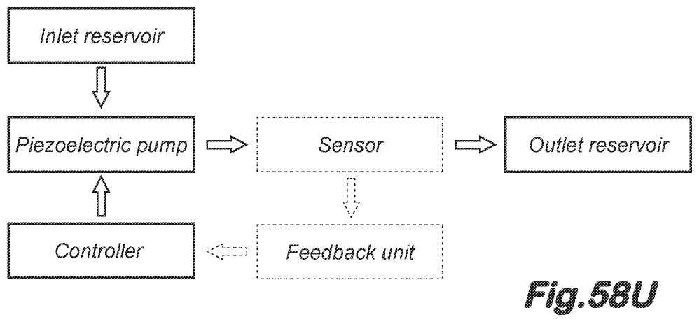

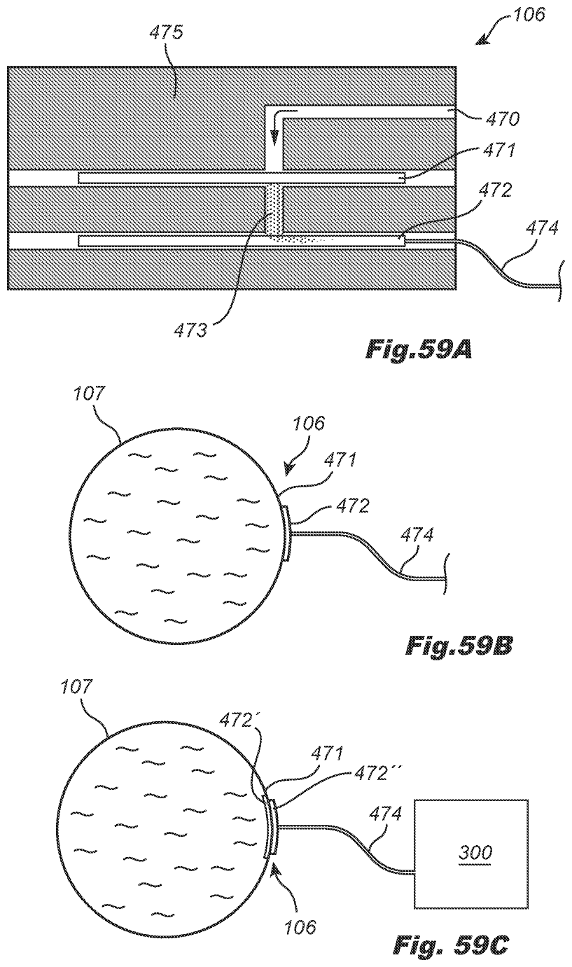

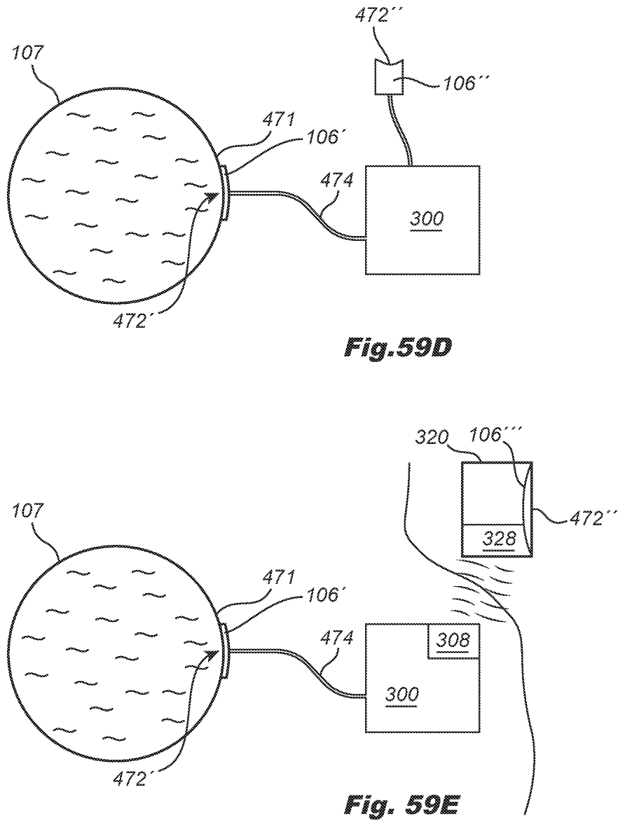

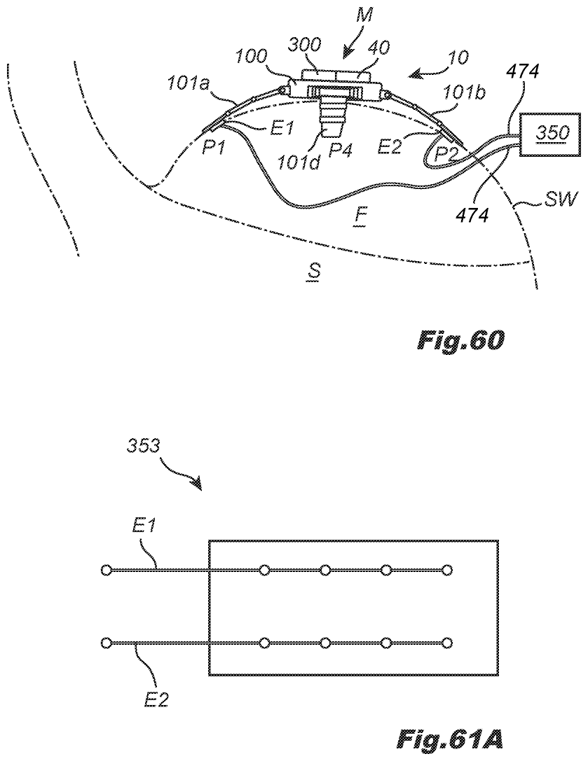

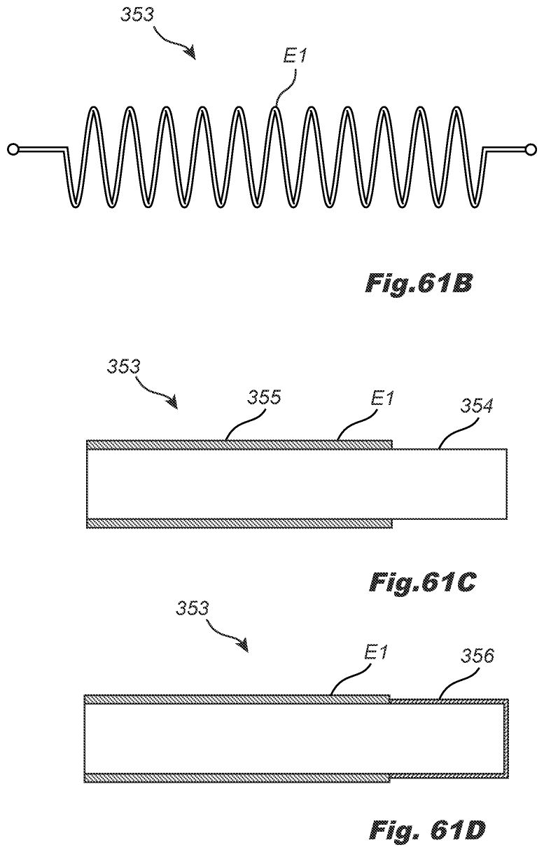

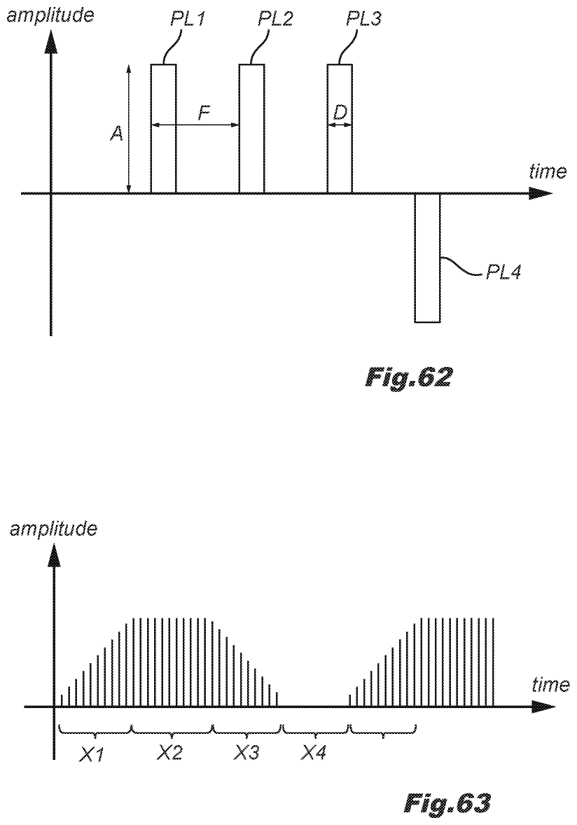

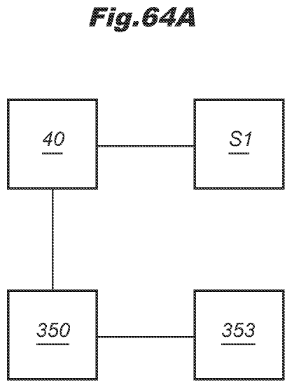

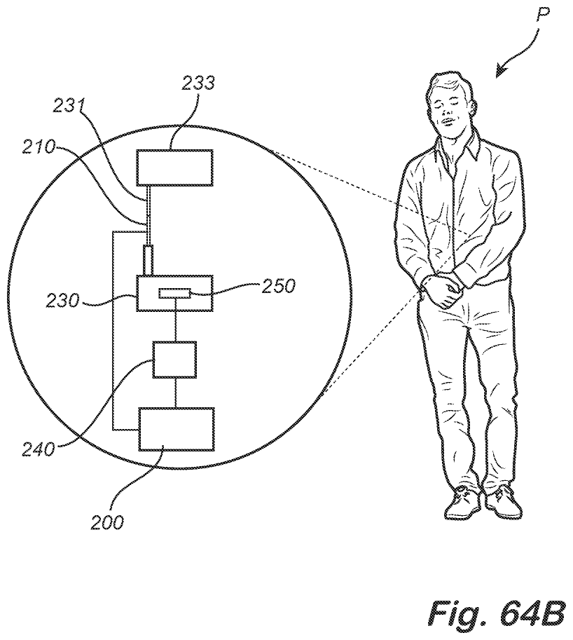

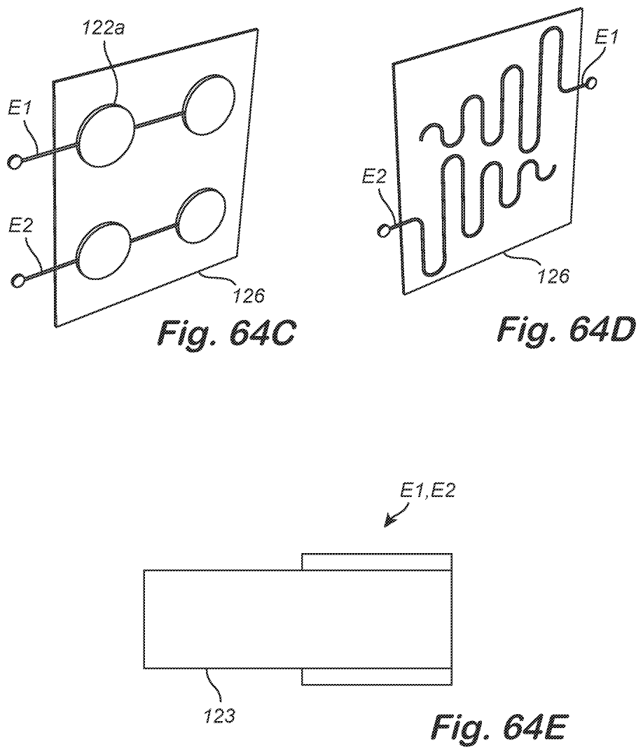

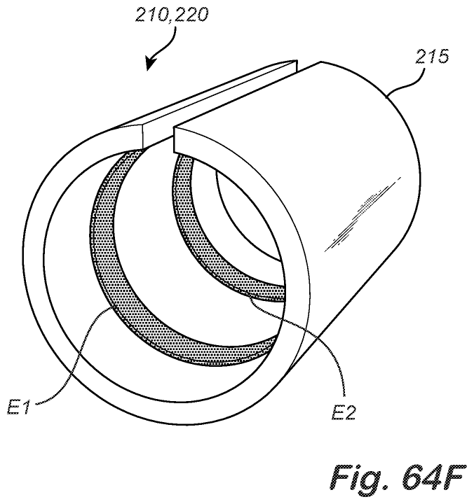

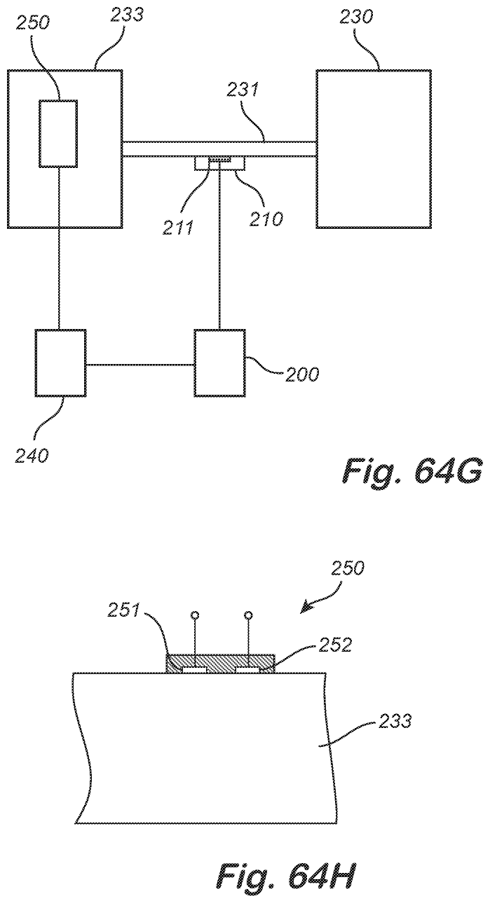

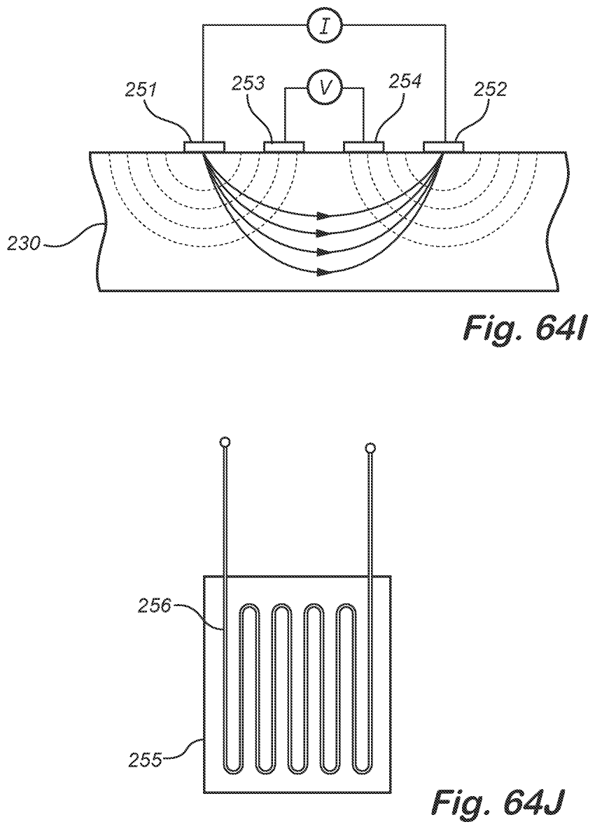

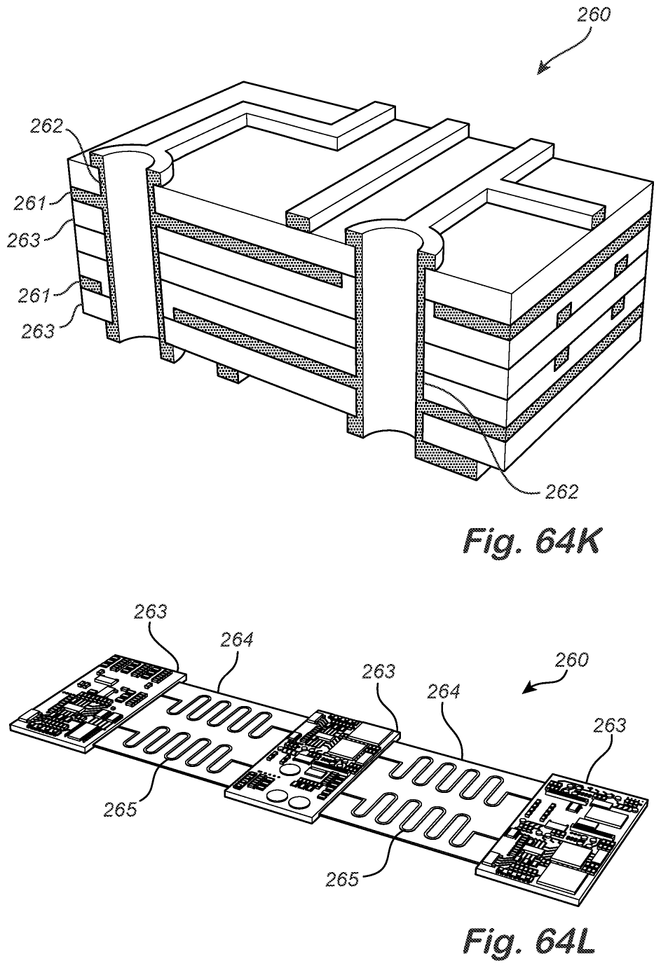

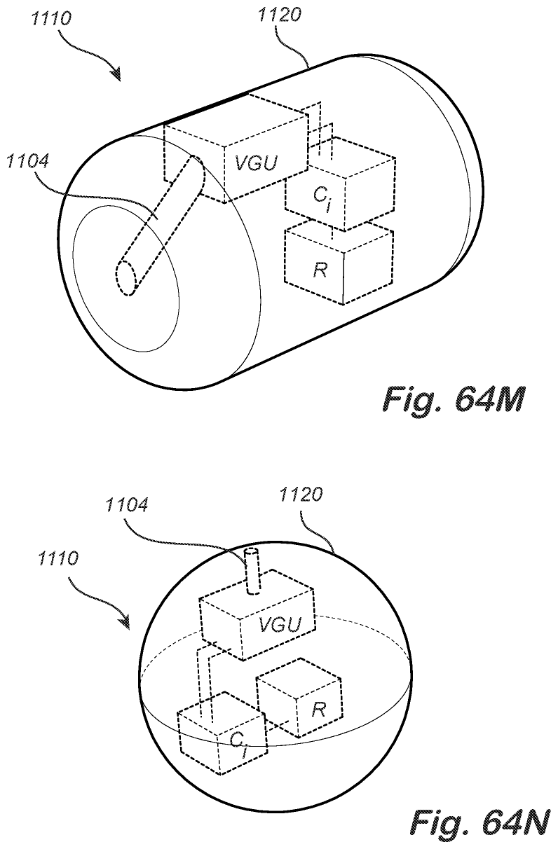

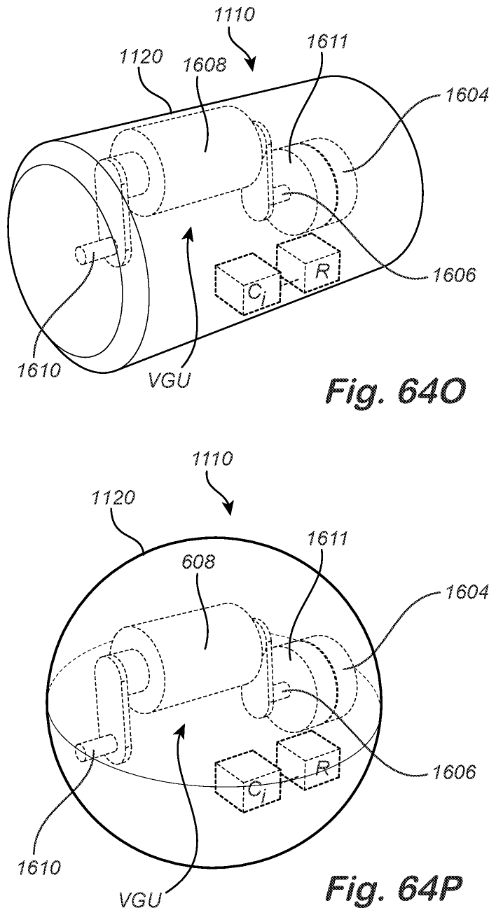

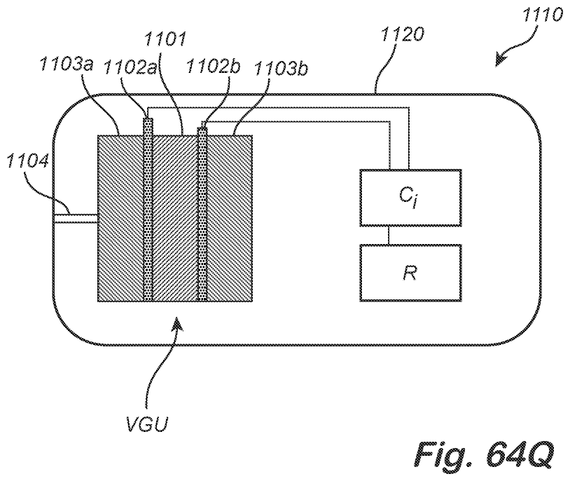

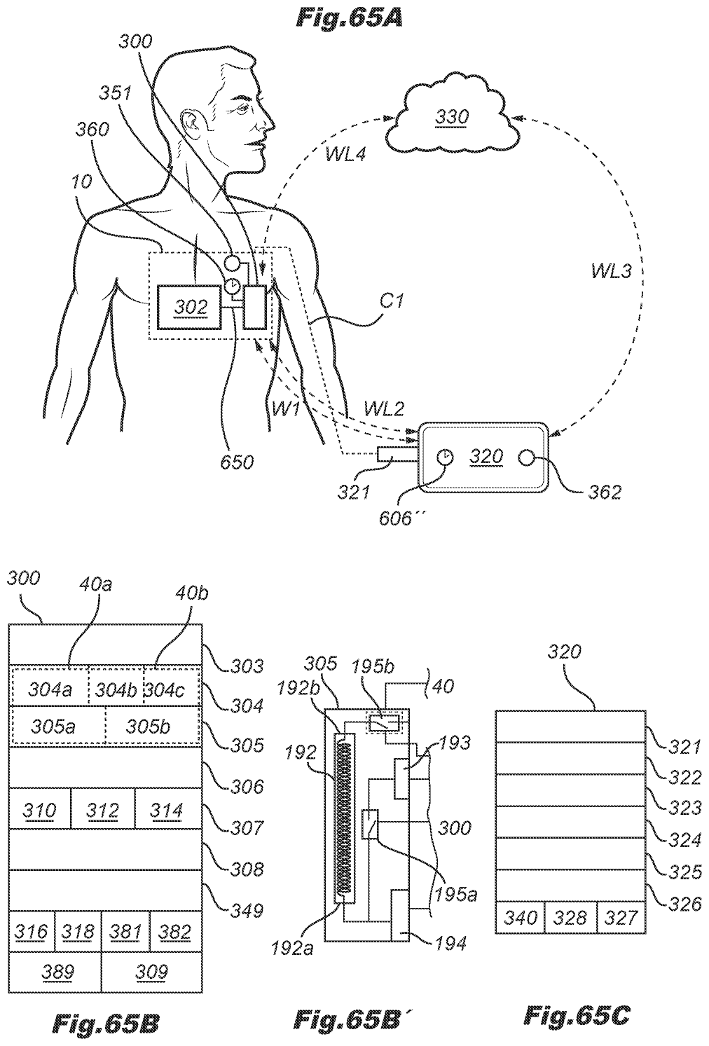

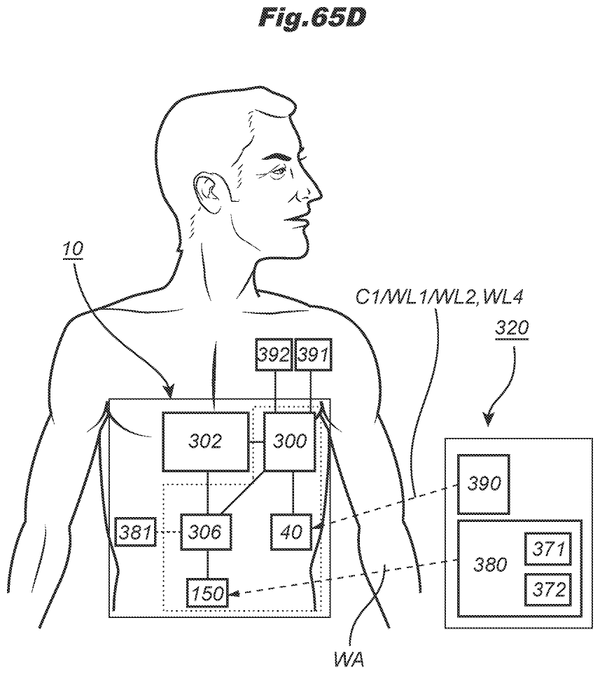

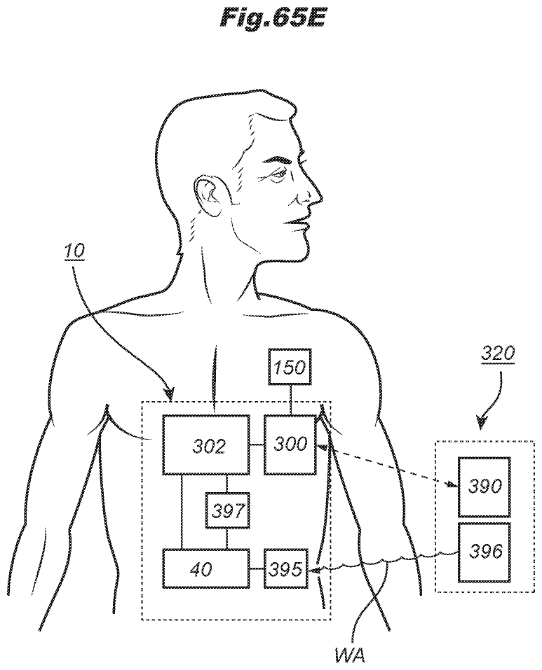

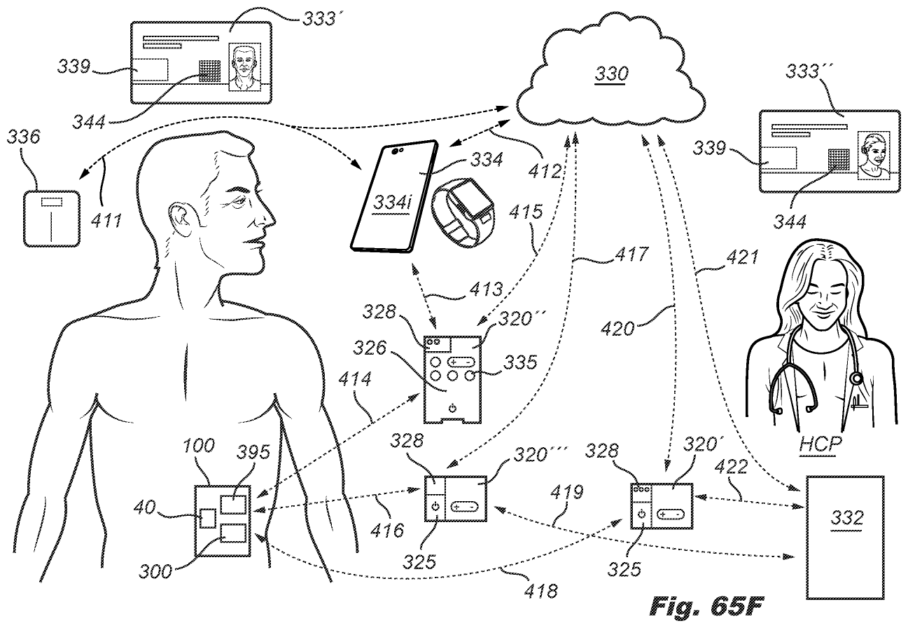

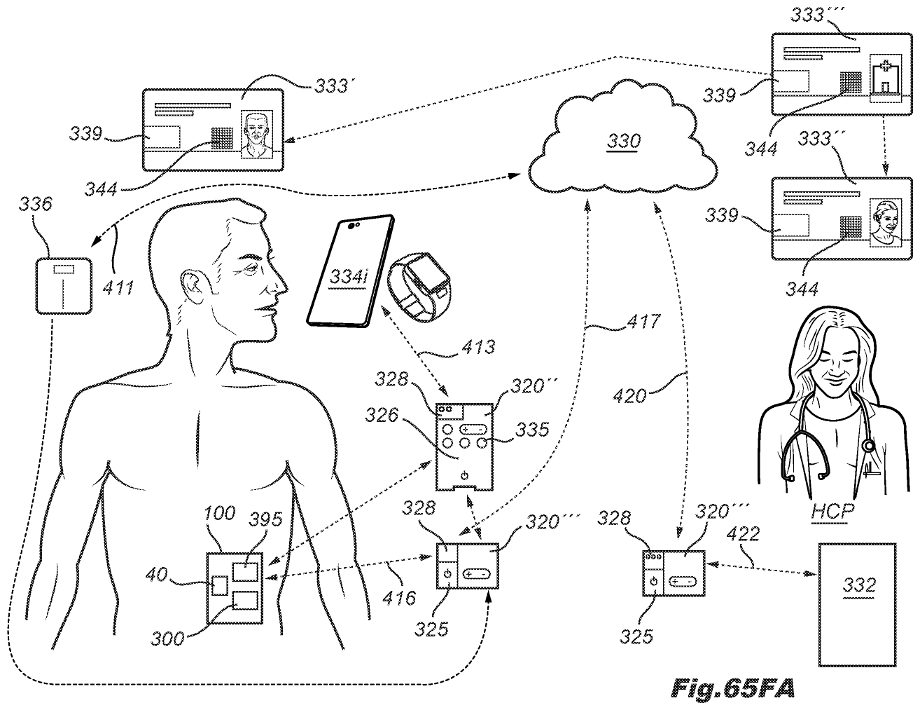

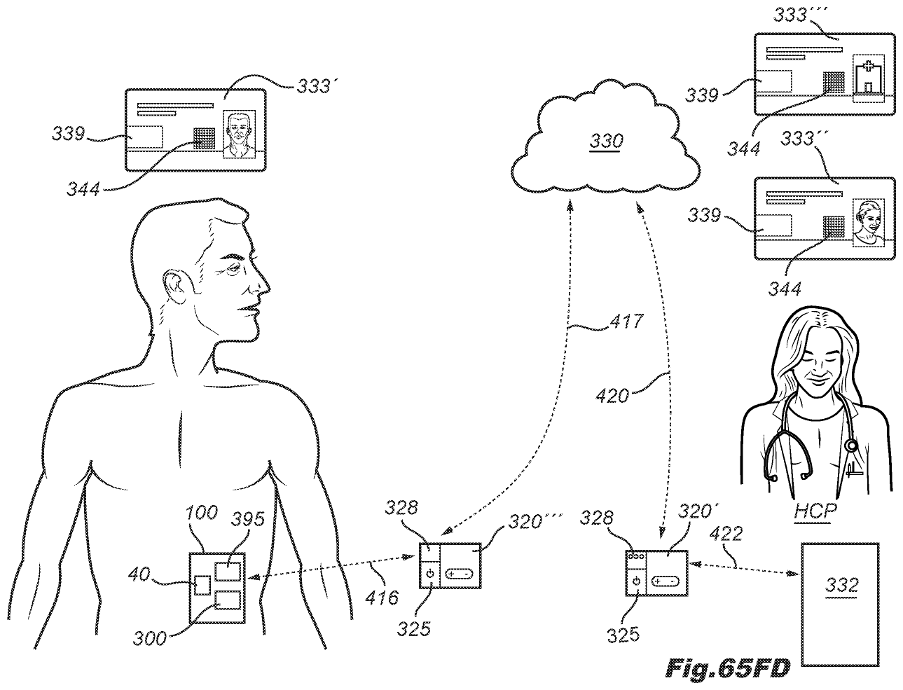

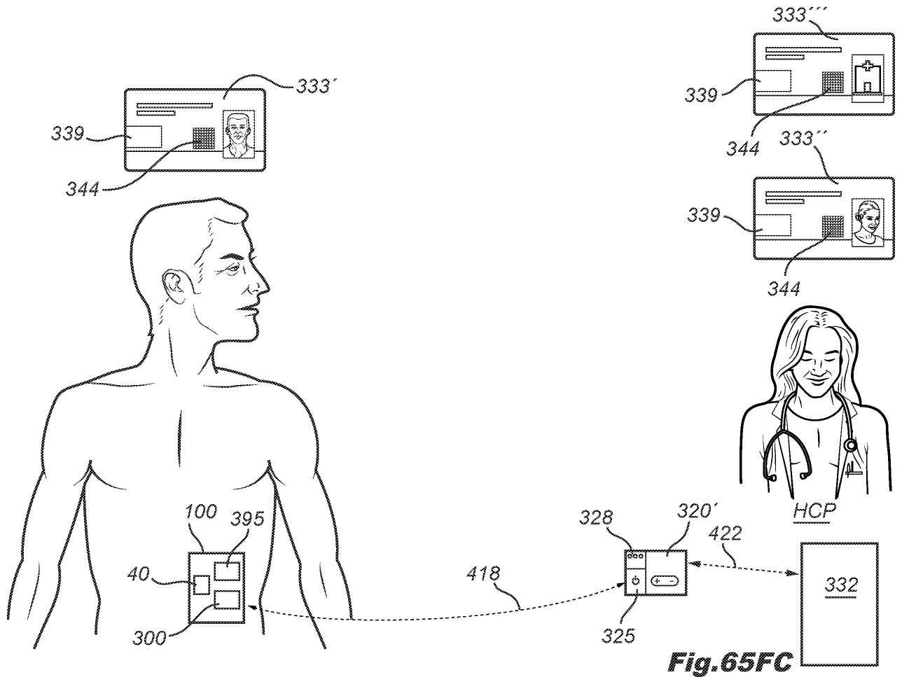

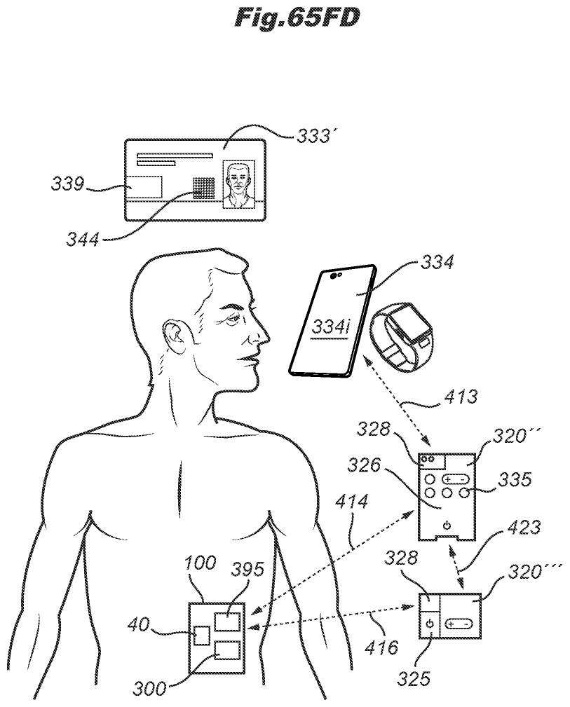

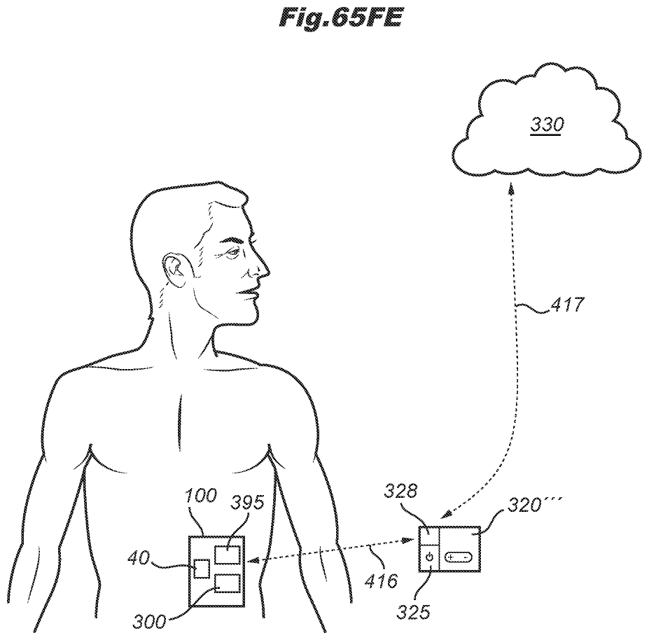

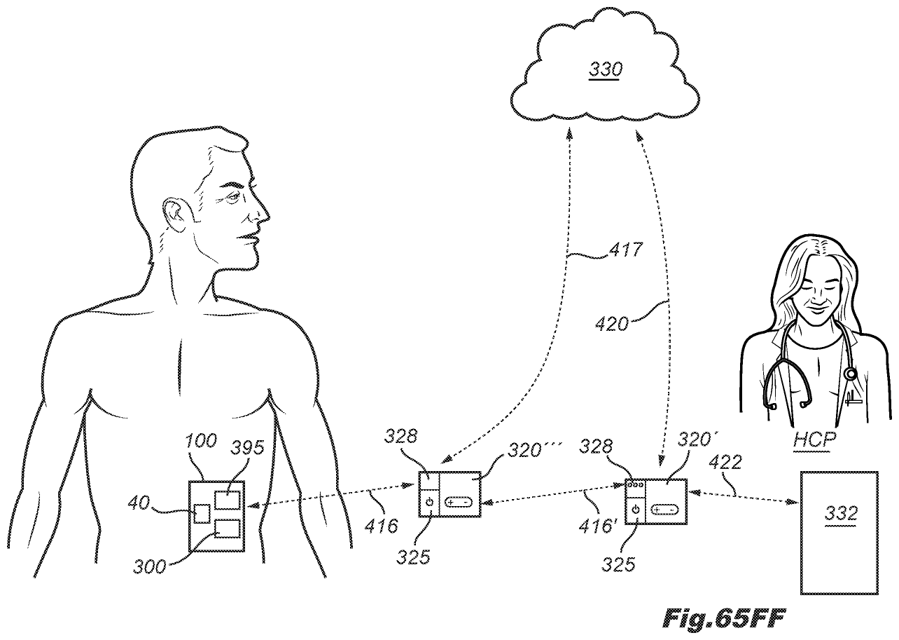

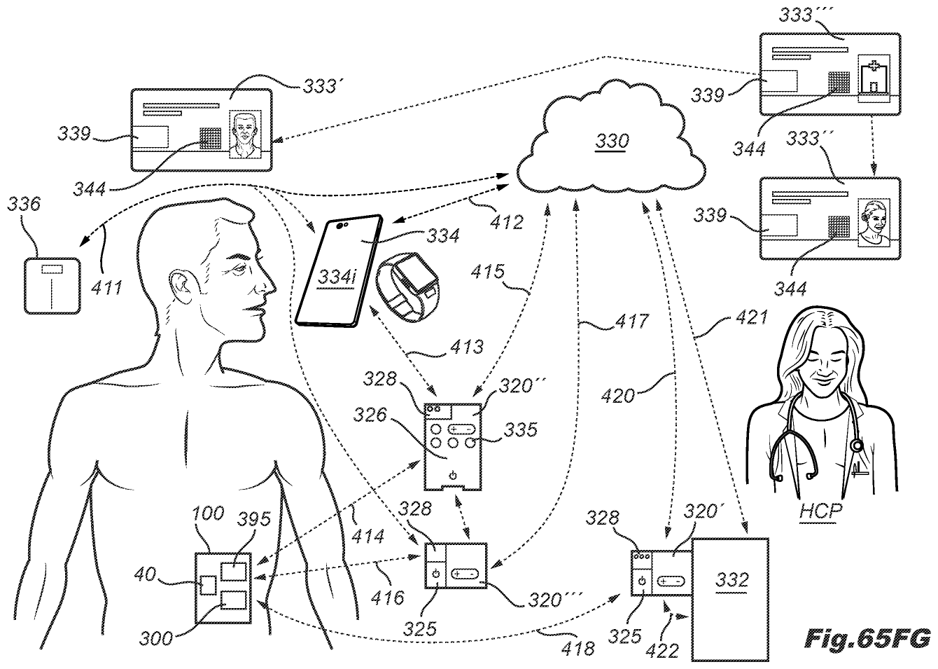

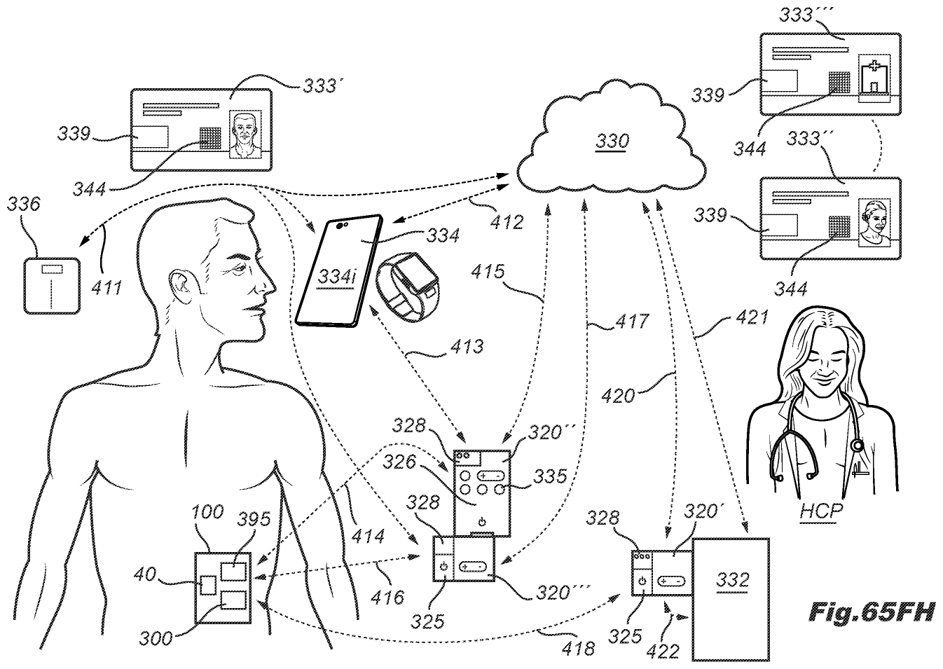

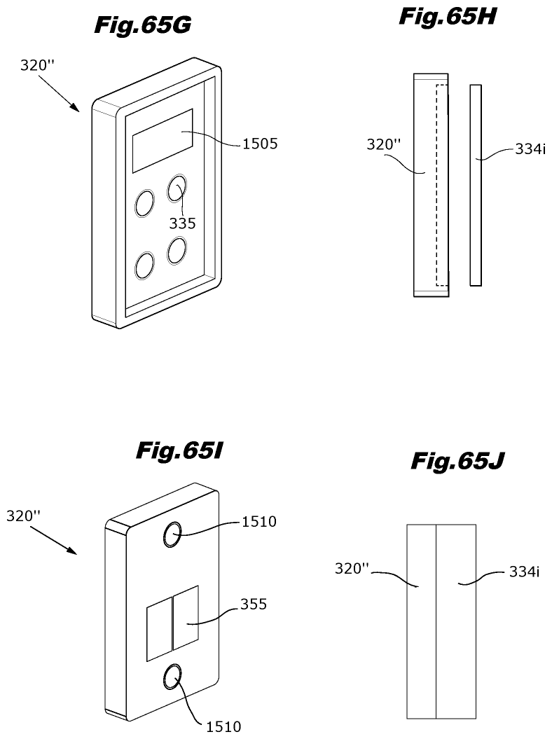

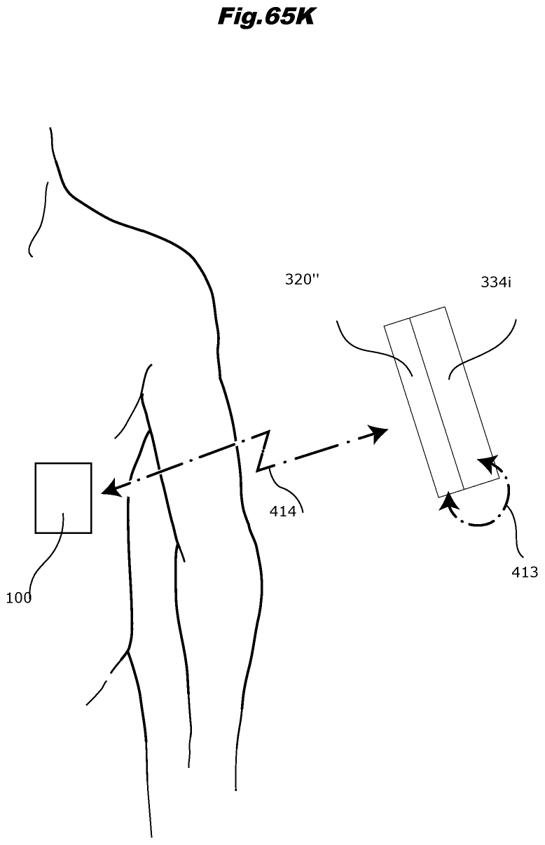

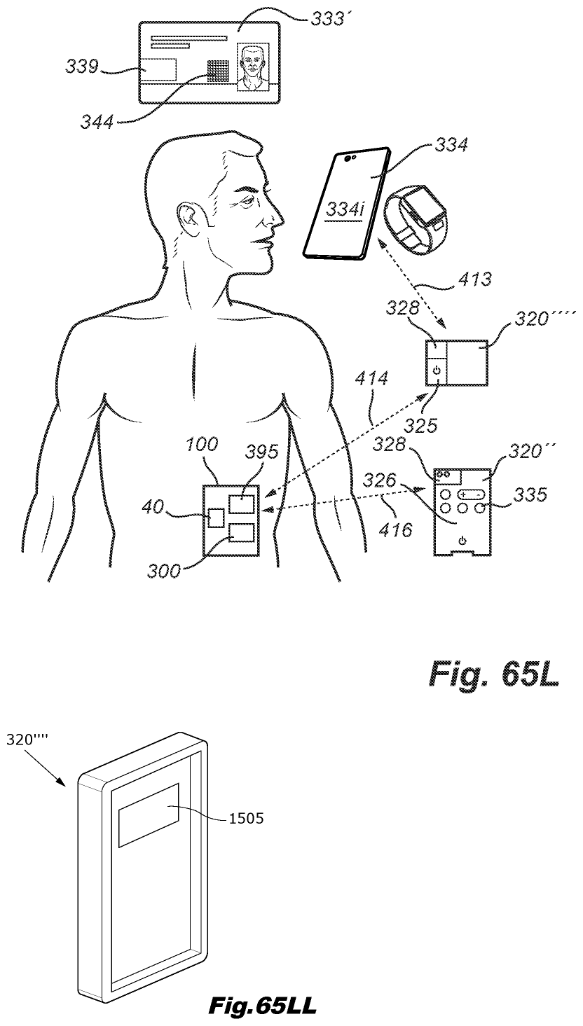

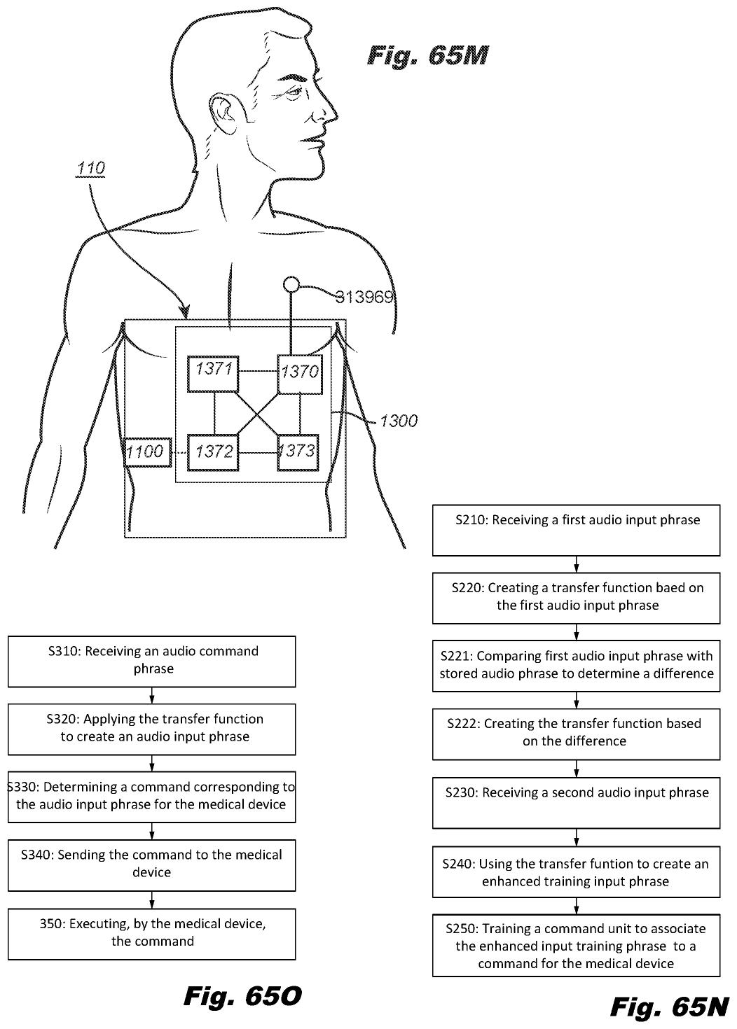

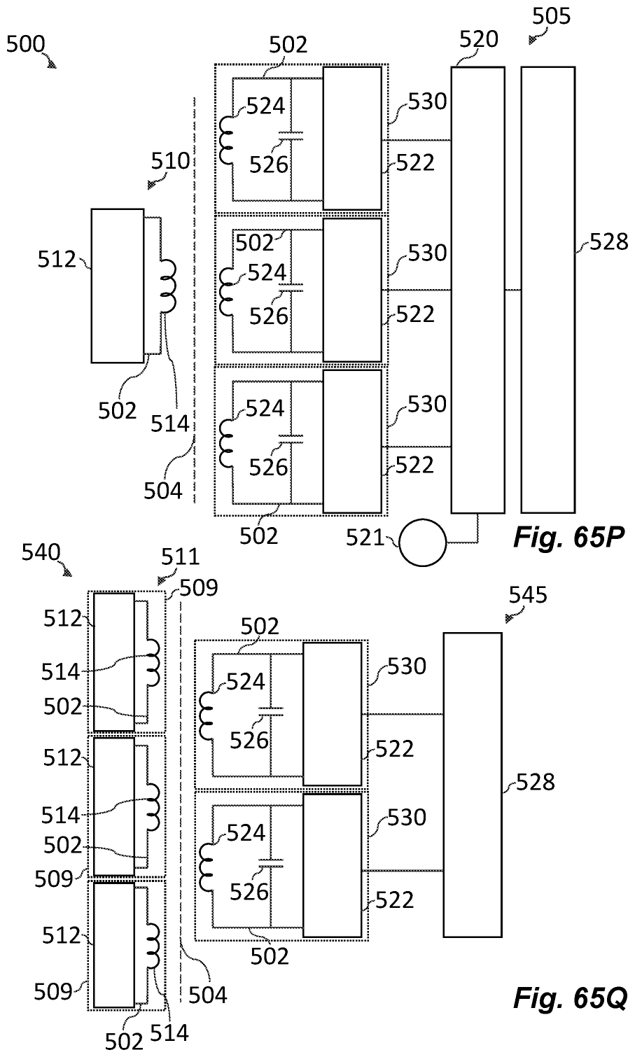

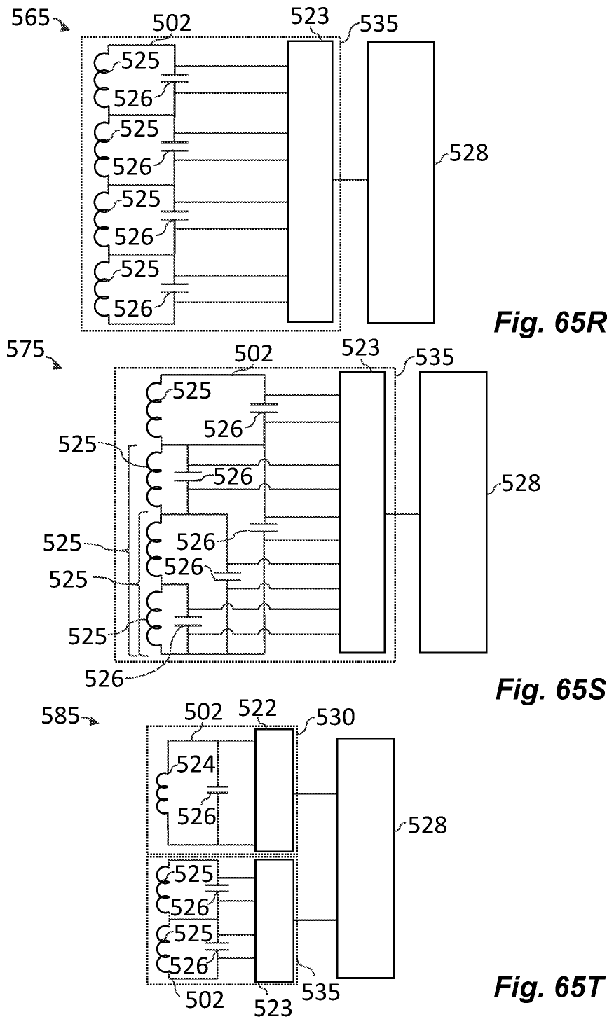

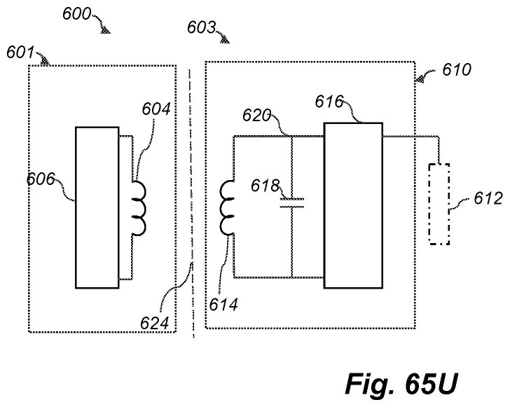

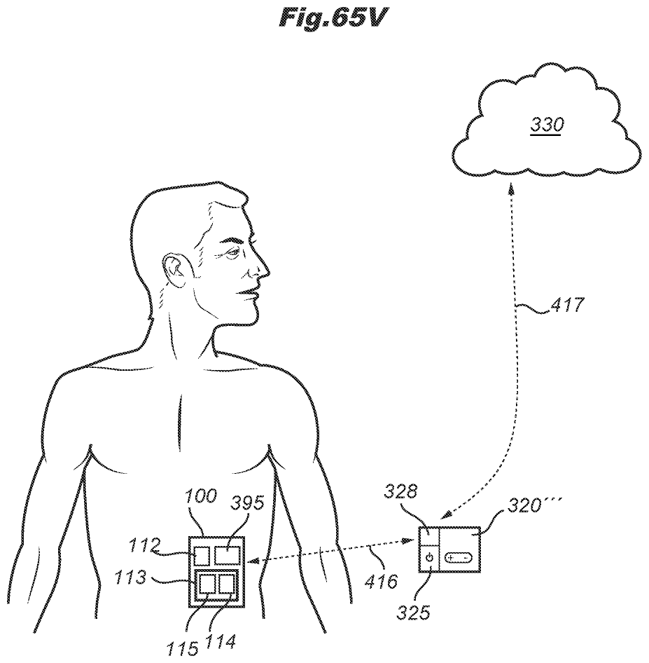

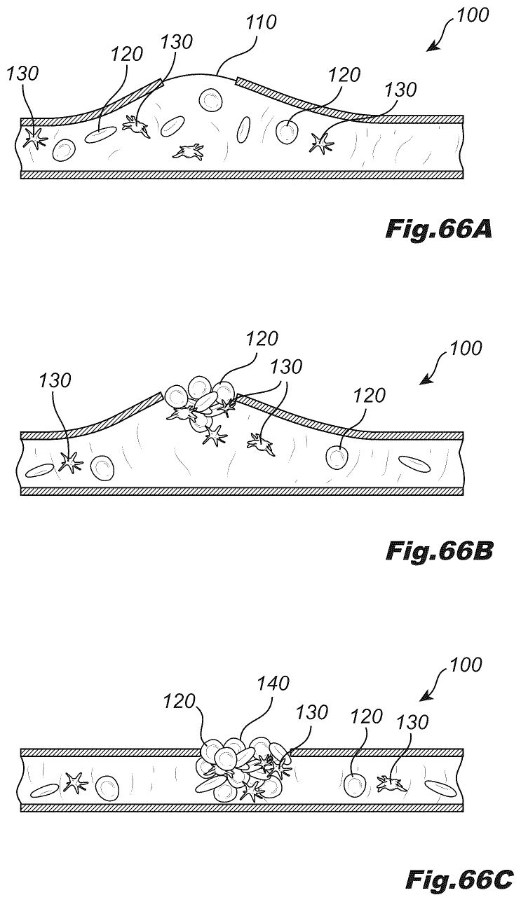

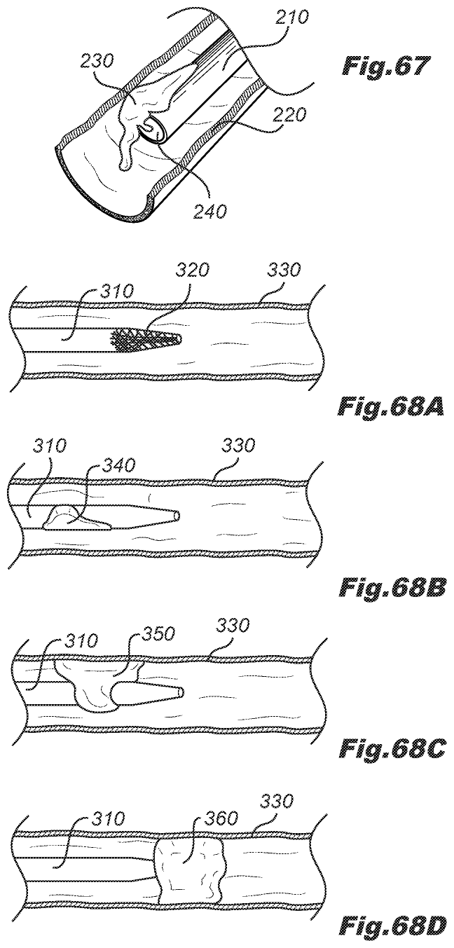

FIGS. 31J-31L′ shows embodiments of members or medical devices which are operable to be calibrated.

FIG. 31M shows an embodiment of a medical device for actively stretching a stomach wall of a patient for creating a sensation of satiety, in an idle state, in plain top view.

FIG. 31M′ shows the medical device of FIG. 31M, in a stretching state, in plain top view.

FIG. 31N shows an embodiment of a medical device for actively stretching a stomach wall of a patient for creating a sensation of satiety, in an idle state, in plain top view.

FIG. 31N′ shows the medical device of FIG. 31N, in a stretching state, in plain top view.

FIG. 31O shows an embodiment of a medical device for actively stretching a stomach wall of a patient for creating a sensation of satiety, in an idle state, in plain top view.

FIG. 31O′ shows the medical device of FIG. 31O, in a stretching state, in plain top view.

FIG. 31P shows an embodiment of a medical device for actively stretching a stomach wall of a patient for creating a sensation of satiety, in a stretching state, in plain top view.

FIGS. 31Q-31T′ schematically shows principles of different embodiments of medical devices for stretching the stomach wall.

FIG. 31U shows an embodiment of a medical device for actively stretching a stomach wall of a patient for creating a sensation of satiety, in an idle state, in plain top view.

FIG. 31U′ shows the medical device of FIG. 31U, in a first stretching state, in plain top view.

FIG. 31U″ shows the medical device of FIG. 31U, in a second stretching state, in plain top view.

FIG. 31V shows an embodiment of a medical device for actively stretching a stomach wall of a patient for creating a sensation of satiety, in an idle state, in plain top view.

FIG. 31V′ shows the medical device of FIG. 31V, in a stretching state, in plain top view.

FIG. 31X shows an embodiment of a medical device for actively stretching a stomach wall of a patient for creating a sensation of satiety, in an idle state, in plain top view.

FIG. 31X′ shows a cross-section of a first portion of the operation device of the medical device of FIG. 31X.

FIG. 31X″ shows a cross-section of a second portion of the operation device of the medical device of FIG. 31X.

FIG. 31X″′ shows the medical device of FIG. 31X, in a stretching state, in plain top view.

FIG. 31Y shows an embodiment of a medical device for actively stretching a stomach wall of a patient for creating a sensation of satiety, in an idle state, in plain top view.

FIGS. 31Y′-31Y″″ shows different embodiments of remote units for operating the medical device for actively stretching a stomach wall of FIGS. 31X-31Y.

FIG. 32A shows an embodiment of a remotely powered medical device for actively stretching a stomach wall of a patient for creating a sensation of satiety, when placed on a stomach.

FIG. 32B shows a frontal view of a human patient in cross section when a remotely powered medical device for actively stretching a stomach wall of a patient has been implanted.

FIG. 33A shows a side view of a medical device comprising a first rotatable member and a second member, the first rotatable member being configured to be rotated by a pulley, the pulley being engaged by a single flexible wire.

FIG. 33A′ shows a top view of the medical device in FIG. 33A.

FIG. 33A″ shows a top view of a medical device similar to that in FIG. 33A, the difference being that the pulley is engaged by two flexible wires.

FIG. 33B shows a side view of a medical device similar to that in FIG. 33A, the difference being that the pulley is biased by a torsion spring towards a rotating direction.

FIG. 33B′ shows atop view of the medical device in FIG. 33B.

FIG. 33C shows a side view of a medical device comprising a first rotatable member, a second member and a third member, the first rotatable member being configured to be rotated by a pulley, the pulley being engaged by a single flexible wire and the pulley being biased by a torsion spring towards a rotating direction.

FIG. 33C′ shows atop view of the medical device in FIG. 33C.

FIG. 33D shows a side view of a medical device similar to the medical device in FIG. 33C, the difference being that the pulley is engaged by two flexible wires and that the torsion spring is absent.

FIG. 33D′ shows a top view of the medical device in FIG. 33D.

FIG. 33E shows a side view of a medical device comprising a first rotatable member and a second rotatable member, the first rotatable member being configured to be rotated by a first pulley, the second rotatable member being configured to be rotated by a second pulley.

FIG. 33E′ shows atop view of the medical device in FIG. 33E.

FIG. 33F shows a side view of a medical device comprising a first rotatable member, a second member and a third rotatable member, the first rotatable member being configured to be rotated by a first pulley and the third rotatable member being configured to be rotated by a third pulley.

FIG. 33F′ shows atop view of the medical device in FIG. 33F.

FIG. 33G shows a side view of a rotatable member of any one of the medical devices in FIGS. 33A-33A′-33A″-33B-33B′-33C-33C′-33D-33D′-33E-33E′-33F-33F′, the rotatable member being partially invaginated.

FIG. 33H shows a medical device being invaginated, at least partially, and comprising a remote unit.

FIGS. 34A-341 shows a medical device for stretching the a wall portion of an intestine.

FIG. 35A shows a cross-sectional view of an implantable remote unit for powering an implantable medical device with hydraulic force.

FIG. 35B shows a cross-sectional view of an implantable remote unit for powering an implantable medical device with hydraulic force.

FIG. 35C shows an exploded cross-sectional view of an implantable remote unit for powering an implantable medical device with hydraulic force.

FIG. 36a shows a detailed cross-sectional view of a first unit of an implantable remote unit for powering an implantable medical device with hydraulic force.

FIG. 36b shows a detailed cross-sectional view of a first unit of an implantable remote unit for powering an implantable medical device with hydraulic force.

FIG. 36c shows a detailed cross-sectional view of a first unit of an implantable remote unit for powering an implantable medical device with hydraulic force.

FIG. 36d shows a detailed cross-sectional view of a first unit of an implantable energized medical device for powering an implantable medical device with hydraulic force.

FIGS. 37a-39b shows alternative embodiments of connecting portions for an implantable remote unit.

FIG. 40 shows, schematically, a kit of components forming an implantable remote unit.

FIG. 41 shows a detailed cross-sectional view of an embodiment of an implantable remote unit for powering an implantable medical device.

FIG. 42 shows a perspective elevated view from the right of an embodiment of an implantable remote unit for powering an implantable medical device.

FIG. 43 shows a perspective elevated view from the right of a portion of an embodiment of an implantable remote unit for powering an implantable medical device.

FIG. 44 shows a perspective elevated view from the right of a portion of an embodiment of an implantable remote unit for powering an implantable medical device.

FIG. 45a shows a cross-sectional plain side view of an embodiment of an implantable remote unit for powering an implantable medical device.

FIG. 45b shows a cross-sectional plain side view of an embodiment of an implantable remote unit for powering an implantable medical device.

FIG. 45c shows a cross-sectional plain side view of an embodiment of an implantable remote unit for powering an implantable medical device.

FIG. 45d shows a cross-sectional plain side view of an embodiment of an implantable remote unit for powering an implantable medical device.

FIG. 46a shows a perspective elevated view from the right of an embodiment of an implantable remote unit for powering an implantable medical device.

FIG. 46b shows a perspective elevated view from the right of an embodiment of an implantable remote unit for powering an implantable medical device.

FIG. 46c shows a perspective elevated view from the right of an embodiment of an implantable remote unit for powering an implantable medical device.

FIG. 46d shows a perspective elevated view from the right of an embodiment of an implantable remote unit for powering an implantable medical device.

FIG. 46e-46q show perspective elevated views from the right of embodiments of an implantable energized medical device for powering an implantable medical device.

FIG. 47 shows a perspective elevated view from the right of an embodiment of an implantable remote unit for powering an implantable medical device.

FIG. 48 shows a plain top view of an embodiment of an implantable remote unit for powering an implantable medical device.

FIGS. 49 and 50 shows, schematically, plain top views of two embodiments of implantable remote units for powering implantable medical devices.

FIGS. 51a-51c illustrates three stages of insertion and fixation of an embodiment of an implantable remote unit for powering an implantable medical device.

FIG. 51d shows a perspective elevated view from the right of an embodiment of an implantable energized medical device for powering an implantable medical device.

FIGS. 51e and 51f show lengthwise cross-sectional areas of the implantable medical device along the line A-A in FIG. 51d.

FIGS. 51g-51i show cross-sectional plain side views of embodiments of an implantable energized medical device for powering an implantable medical device.

FIG. 51j shows a perspective elevated view from the right of an embodiment of an implantable energized medical device for powering an implantable medical device

FIGS. 51k and 511c show lengthwise cross-sectional areas of the implantable medical device along the line A-A in FIG. 51j.

FIG. 51m shows a cross-sectional plain side view of an embodiment of an implantable energized medical device.

FIGS. 51n-51p show cross-sectional plain side views of an embodiment of an implantable energized medical device.

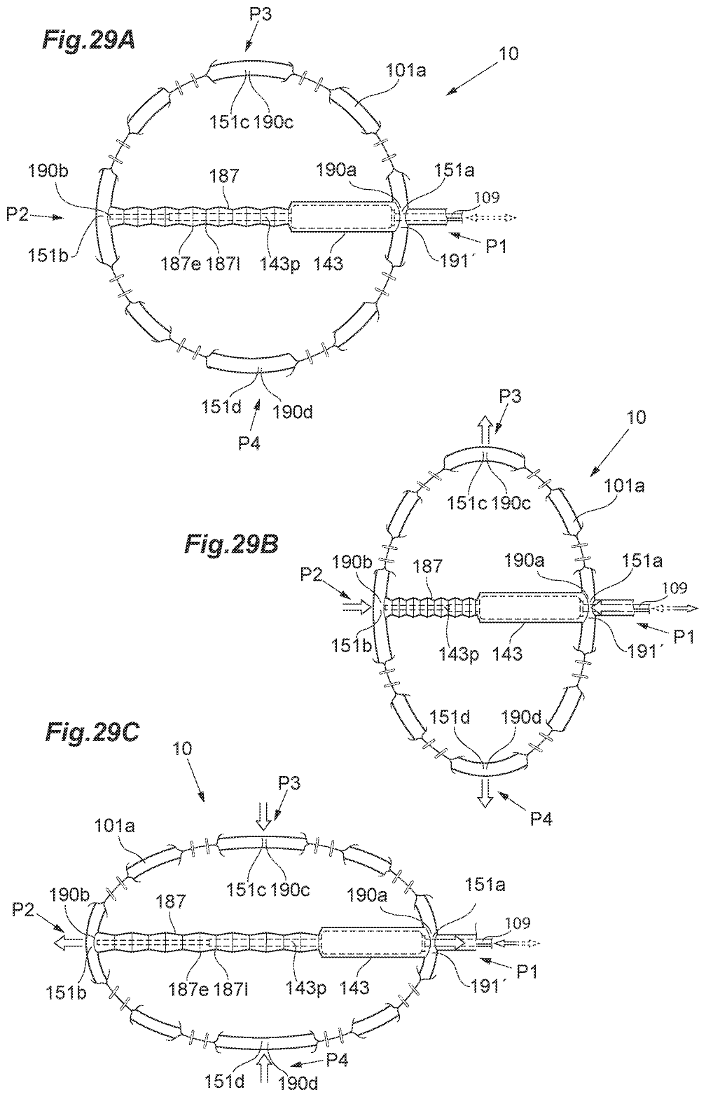

FIGS. 51q-51t shows cross-sectional plain side views of the embodiment in FIGS. 29a-29c when inserted into a tissue portion.

FIGS. 51u-51z show cross-sectional plain side views of embodiments of an implantable energized medical device.

FIG. 51aa shows an embodiment of an implantable energized medical device for powering an implantable medical device or body engaging portion.

FIGS. 51ab-51aq show schematic cross-sectional plain side views of systems comprising an implantable energized medical device.

FIG. 51ar-51au show cross-sectional plain side views of embodiments of an implantable energized medical device.

FIG. 52 shows a detailed cross-sectional view of an embodiment of an implantable remote unit for powering an implantable medical device.

FIG. 53a shows, schematically, a portion of an implantable remote unit for powering an implantable medical device.

FIG. 53b shows, schematically, a portion of an implantable remote unit for powering an implantable medical device.

FIG. 53c shows, schematically, a portion of an implantable remote unit for powering an implantable medical device.

FIG. 54a shows a plain view of an embodiment of a hydraulic pump for operating an implantable medical device.

FIG. 54b shows a side view of the hydraulic pump of FIG. 54a, for operating an implantable medical device.

FIG. 55a shows a top view of a gear system for an implantable medical device.

FIG. 55b shows a partially sectional side view of a gear system for an implantable medical device.

FIG. 56 shows a sectional side view of an embodiment of a hydraulic pump for an implantable medical device.

FIG. 57a shows a sectional side view of an embodiment of a hydraulic pump for an implantable medical device.

FIG. 57b shows a partially sectional perspective view from the left of an embodiment of a hydraulic pump for an implantable medical device.

FIG. 57c shows a partially sectional perspective view from the left of an embodiment of a hydraulic pump for an implantable medical device.

FIG. 57d shows a partially sectional perspective view from the left of an embodiment of a hydraulic pump for an implantable medical device.

FIG. 57e shows a partially sectional perspective view from the left of an embodiment of a hydraulic pump for an implantable medical device.

FIG. 57f shows a partially sectional perspective view from the left of an embodiment of a hydraulic pump for an implantable medical device.

FIG. 57g shows a partially sectional perspective view from the left of an embodiment of a hydraulic pump for an implantable medical device.

FIG. 57h shows a partially sectional perspective view from the left of an embodiment of a hydraulic pump for an implantable medical device.

FIG. 57i shows an elevated perspective view from the left of an embodiment of a hydraulic pump for an implantable medical device.

FIGS. 58A-58H shows different embodiments of piezoelectric motors which may be used for operating the implantable medical devices described herein.

FIGS. 581-58U shows different embodiments of piezoelectric pumps which may be used for operating hydraulic embodiments of implantable medical devices described herein.

FIG. 59a shows an embodiment of a sensor for sensing the pressure in a hydraulic portion of the implantable medical device.

FIG. 59b shows an embodiment of a sensor for sensing the pressure in a hydraulic portion of the implantable medical device.

FIG. 59c shows an embodiment of a sensor for sensing the pressure in a hydraulic portion of the implantable medical device.

FIG. 59d shows an embodiment of a sensor for sensing the pressure in a hydraulic portion of the implantable medical device.

FIG. 59e shows an embodiment of a sensor for sensing the pressure in a hydraulic portion of the implantable medical device.

FIG. 60 shows an embodiment of a medical device for actively stretching a stomach wall of a patient for creating a sensation of satiety, including an electrode arrangement for electrical stimulation, when placed on a stomach.

FIG. 61a shows an embodiment of an electrode arrangement, for inclusion in an implantable medical device.

FIG. 61b shows an embodiment of an electrode arrangement, for inclusion in an implantable medical device.

FIG. 61c shows an embodiment of an electrode arrangement, for inclusion in an implantable medical device.

FIG. 61d shows an embodiment of an electrode arrangement, for inclusion in an implantable medical device.

FIG. 62 shows an embodiment of a stimulation cycle for electrical stimulation of a tissue wall.

FIG. 63 shows an embodiment of a stimulation cycle for electrical stimulation of a tissue wall.

FIG. 64A is a block diagram schematically describing the function of the system for electrical stimulation of a tissue wall of the patient.

FIGS. 64B-64J show embodiments and describes various functions of a system for stimulating the tissue wall of a stomach or intestine.

FIGS. 64K-64L show embodiments of PCBs for the medical device.

FIGS. 64M-64Q show embodiments of vibrators to be used in the medical device.

FIGS. 65A-65H show an embodiment and describes various functions of an implantable controller for controlling the implantable medical device.

FIG. 65G shows an elevated perspective view from the left of a housing unit.

FIG. 65H shows a plain view from the left of a housing unit.

FIG. 651 shows an elevated perspective view from the left of a housing unit.

FIG. 65J shows a plain view from the left of a housing unit.

FIG. 65K shows a system overview of an external device comprising a housing unit and a display device in wireless communication with an implanted medical device.

FIG. 65L shows a system having a first and a second remote control.

FIG. 65LL shows the second remote control comprised in a housing unit.

FIG. 65M schematically shows a medical implant when implanted in a patient.

FIG. 65N shows a flow chart for a method for training a medical implant to recognize a voice command, according to some embodiments;

FIG. 650 shows a flow chart for a method for using voice commands to control a medical implant, according to some embodiments.

FIG. 65P-65T illustrates implantable medical devices and external devices for transferring wireless energy to the implantable medical devices.

FIG. 65U illustrates an implantable medical device and an external device configured to transmit data using near field magnetic induction.

FIG. 65V illustrates an embodiment for communication with an implantable medical device.

FIGS. 66A-66C describes the reaction that takes place when a blood vessel is damaged.

FIG. 67 shows a portion of an implantable medical device placed inside a vein with a fibrin sheath that has formed on and around part of the medical device.

FIGS. 68A-68D shows the formation of a blood clot in a vein.

FIG. 69 shows an implantable medical device comprising an implant surface and a coating arranged on the surface.

FIG. 70 shows an exemplary implantable medical device comprising an at least partially hollow implant body.

FIG. 71 shows an exemplary implantable medical device with a surface.

FIGS. 72A and 72B shows different micropatterns on the surface of an implant.

### DETAILED DESCRIPTION

In the following a detailed description of embodiments of the invention will be given with reference to the accompanying drawings. It will be appreciated that the drawings are for illustration only and are not in any way restricting the scope of the invention. Thus, any references to directions, such as “up” or “down”, are only referring to the directions shown in the figures. It should be noted that the features having the same reference numerals have the same function, a feature in one embodiment could thus be exchanged for a feature from another embodiment having the same reference numeral unless clearly contradictory. The descriptions of the features having the same reference numerals should thus be seen as complementing each other in describing the fundamental idea of the feature and thereby showing the features versatility.

A controller is to be understood as any implantable unit capable of controlling the restriction device. A controller could include a motor and/or pump or another operation device for operating the implantable hydraulic restriction device or could be separate from the operation device and only be adapted to control the operation thereof. A control signal is to be understood as any signal capable of carrying information and/or electric power such that the restriction device can be directly or indirectly controlled.

Implantable operation device is to be understood as any device or system capable of operating an active implant. An operation device could for example be an actuator such as a hydraulic actuator such as a hydraulic pump or a hydraulic cylinder, or a mechanical actuator, such as a mechanical element actuating an implant by pressing or pulling directly or indirectly on the implant, or an electro-mechanical actuator such as an electrical motor or solenoid directly or indirectly pressing or pulling on the implant.

A gear system is to be understood as any system capable of providing transmission such that work of a first form can be transmission into work of a second form. The form of the work could for example include the speed, the force and/or the direction of the work.

All foreign matter implanted into the human body inevitably causes an inflammatory response. In short, the process starts with the implanted medical device immediately and spontaneously acquiring a layer of host proteins. The blood protein-modified surface enables cells to attach to the surface enabling monocytes and macrophages to interact on the surface of the medical implant. The macrophages secrete proteins that modulate fibrosis and in turn developing the fibrosis capsule around the foreign body. In practice, a fibrosis capsule is a dense layer of excess fibrous connective tissue. On a medical device implanted in the abdomen, the fibrotic capsule typically grows to a thickness of about 0.5 mm-2 mm, and is substantially inelastic and dense. As the fibrotic tissue is substantially inelastic, this means that all operable elements that are supposed to move when implanted in the body (such as the members and enclosures of the medical devices disclosed herein) needs a shape such that it is substantially unaffected by the formation of an inelastic layer of fibrotic tissue on its surface. In some of the embodiments, this is solved by having movable parts and enclosures having elevated and lowered portions, such that the distance covered by fibrotic tissue becomes longer and bellows type expansion or bending is enabled. The distance between the “peaks” of two elevated portions may be in the interval 2 mm-6 mm, or in the interval 2 mm-5 mm and the elevation difference between the lowest point of a lowered portion to a peak may be in the interval 1 mm-6 mm, or in the interval 1 mm-5 mm.

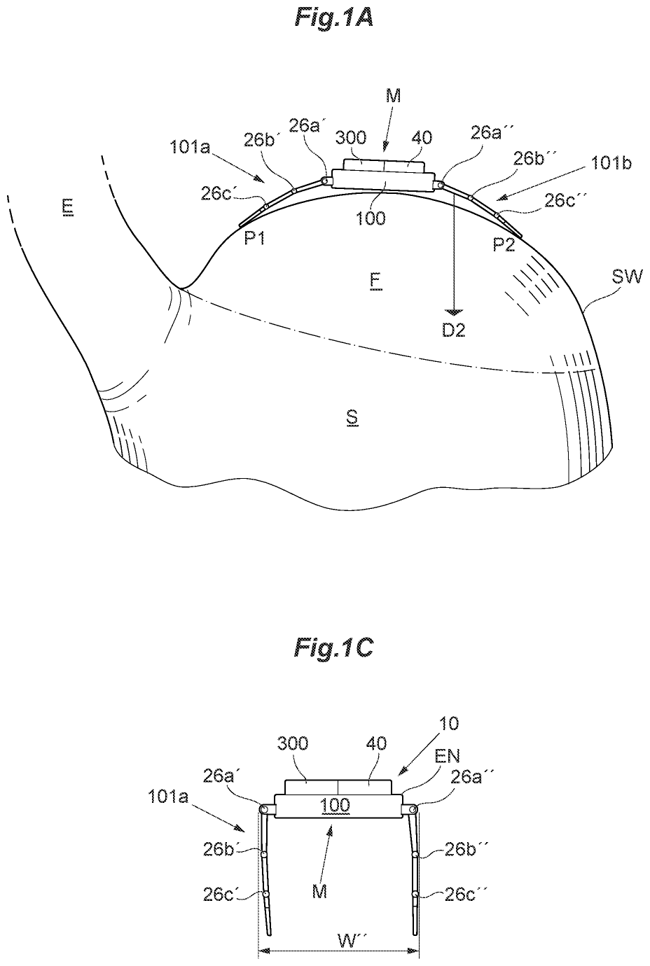

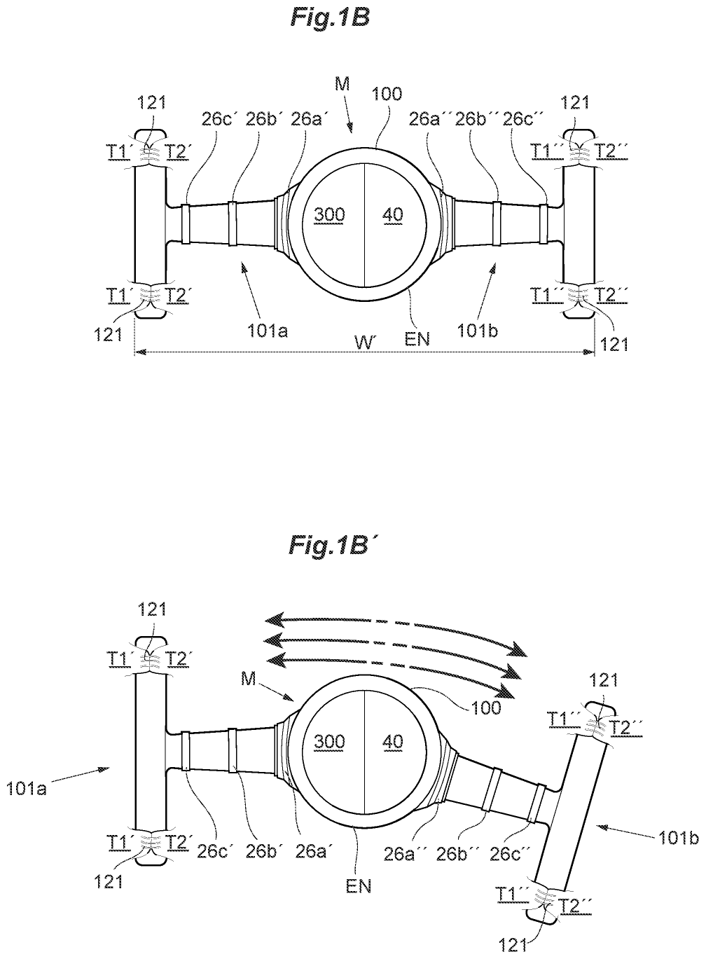

FIG. 1a shows an embodiment of a medical device 10 for actively stretching a stomach wall SW of a patient for creating a sensation of satiety. The medical device 10 is implantable and comprises a first member 101a, which in the embodiment of FIGS. 1a-1c is elongated and configured to be fixated to a first portion P1 of the stomach wall SW and a second member 101b, which in the embodiment of FIGS. 1a-1c is elongated and configured to be fixated to a second portion P2 of the stomach wall SW. The medical device 10 further comprises an operation device 100 for operating the second member 101b to displace the second member 101b in a first direction D1 relative to the first member 101a, for stretching the stomach wall SW between the first and second portion P1, P2. At least one of the first and second member 101a and 101b is flexible in a second direction D2 substantially perpendicular to the first direction D1 for adapting the medical device 10 to the curvature of the stomach S of the patient and/or for facilitating insertion of the medical device 10 into the body of the patient.

In the embodiment shown in FIGS. 1a-1c, the operation device 100 is further configured for operating the second member 101b to displace the second member in a third direction D3 relative to the first member, for stretching the stomach wall between the first and second portion P1, P2.

In the embodiment shown in FIGS. 1a and 1b, the first member 101a is inoperably fixated to a main portion M which houses the operation device 100, and the second member is operably fixated to a main portion M.

In the embodiment shown in FIGS. 1a-1c, the first and second members 101a, 101b are hinged for enabling pivotal movement. In the embodiment shown in FIGS. 1a-1c the first and second members 101a, 101b are hinged at a first, second and third pivotal points 26a′,26b′,26c′, 26a″,26b″,26c″, which enables the first and second member 101a, 101b to adapt to the curvature of the stomach S following the curvature of the fundus F part of the stomach wall SW.

In the embodiment shown in FIGS. 1a-1c, the first and second member 101a, 101b member is configured to be partially invaginated by the tissue T1′, T2′, T1″, T2″ of the stomach wall SW, such as shown in FIGS. 1a and 1b. Invagination means that tissue T1′, T2′, T1″, T2″ of the stomach wall SW is folded over the first and second members 101a, 101b and sutured 121 or stapled 121 together. This means that first tissue T1′, T1″ connects to second tissue T2′, T2″ over the first and second members 101a, 101b, making it possible for the connected tissue T1′, T2′, T1″, T2″ of the stomach wall SW to grow together creating a stable and reliable long term fixation even if the sutures 121 or staplers 121 are resorbed or rejected by the body.

In the embodiment shown in FIGS. 1a-1c, the main portion M comprises an enclosure EN enclosing the operation device 100. The enclosure EN is flexible to enable the displacement of the second member 101b in a first direction D1 relative to the first member 101a, for stretching the stomach wall SW between the first and second portion P1, P2.

In the embodiment shown in FIGS. 1a-1c, the main portion M comprise an energy storage unit 40 for directly or indirectly energizing the medical device 10. The implantable energy storage unit 40 may be any type of energy storage unit suitable for an implant, such as a re-chargeable battery or a solid-state battery, such as a tionyl-chlorid battery and may comprise a receiver for receiving energy wirelessly. The energy storage unit may be equipped with an energy storage unit indicator configured to indicate a functional status of the implantable energy storage unit. The functional status may indicate at least one of charge level and temperature of the implantable energy storage unit 40. For enabling indication of the temperature of the energy storage unit 40, the energy storage unit 40 or medical device 10 may comprising a temperature sensor.

In the embodiment shown in FIGS. 1a-1c, the main portion M further comprises a controller 30 for controlling the operation device 100. The controller connects the energy storage unit 40 to the operation device 100, such that the controller can control the energy provided to the operation device and thereby control the operation device and the stretching of the stomach wall. 38. The medical device according to claim 37, wherein the controller comprises a wireless transceiver for communicating wirelessly with a source external to the body of the patient.

The controller 300 may comprise at least one sensor, or be configured to receive sensor input from the at least one sensor. The sensor could be a sensor configured to sense a physical parameter of the medical device system, such as at least one of:

A temperature of the medical device system, to avoid excessive heating of tissue connected to the medical device during operation of the medical device or charging of the energy storage unit 40. Excessive heating may also damage the medical device and/or the energy storage unit 40. Excessive heating may also be an indicator that something is wrong with the medical device 10 and may be used for triggering an alarm function for alerting the patient or physician.

A parameter related to the power consumption of the medical device system, to avoid excessive power consumption which may drain and/or damage the energy storage unit 40. Excessive power consumption may also be an indicator that something is wrong with the medical device 10 and may be used for triggering an alarm function for alerting the patient or physician.

A parameter related to strain in the medical device, such as the strain in the operation device 100 or first or second member 101a, 101b relate to the operation of the medical device. Strain can be measured to avoid excessive strain which may damage medical device or the tissue of the patient. Excessive strain may also be an indicator that something is wrong with the medical device 10 and may be used for triggering an alarm function for alerting the patient or physician. It may be important to measure strain or pressure in or exerted by the medical device, as too high strain or pressure risks hampering the blood flow to the tissue of the stomach wall, which in the long term could lead to damage of the tissue and in the worst-case lead to necrosis.

A parameter related to the wireless transfer of energy from a source external to the body of the patient. Excessive transfer of wireless energy may damage an implanted wireless energy received or the energy storage unit. It may also create excessive heating which may damage the tissue of the patient.

In embodiments in which the operation device is a hydraulic operation device, the sensor may be a sensor configured to sense a hydraulic pressure. Hydraulic pressure can be measured to avoid excessive hydraulic pressure which may damage medical device or the tissue of the patient. Excessive hydraulic pressure may also be an indicator that something is wrong with the medical device 10 and may be used for triggering an alarm function for alerting the patient or physician.

The controller 300 may comprise a sensor or be configured to receive sensor input from a sensor configured to sense a physiological parameter of the patient. The physiological parameter of the patient may be:

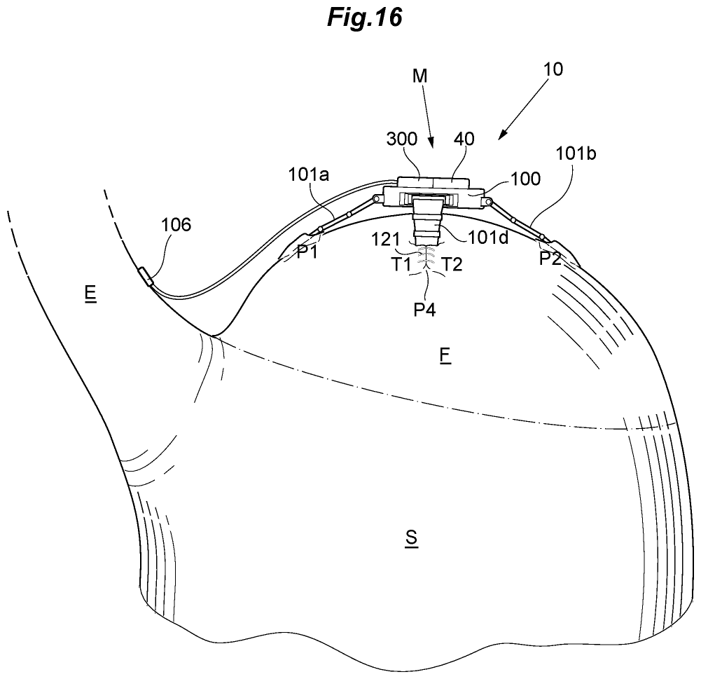

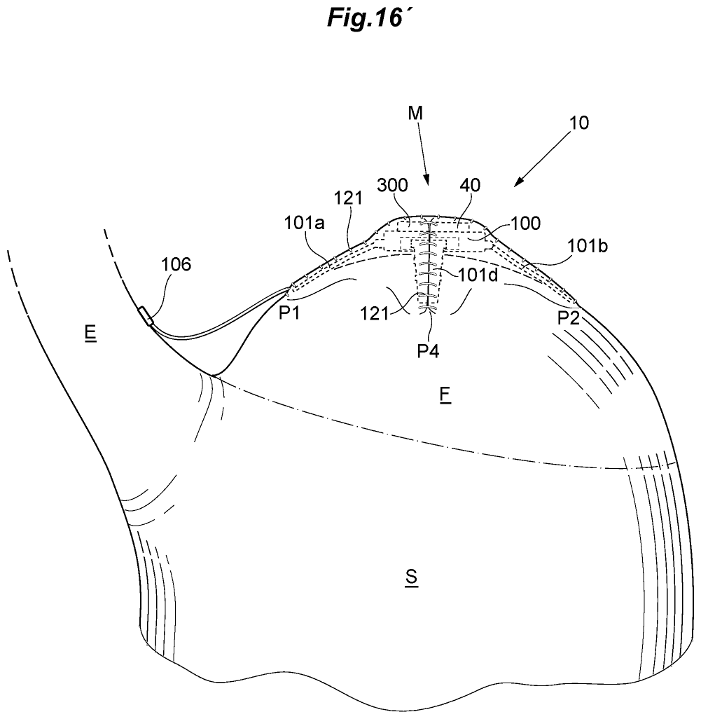

A parameter related to the patient swallowing, such that the medical device can be controlled on the basis of the patient eating or drinking. A sensor configured to sense a parameter related to the patient swallowing could comprises a motility sensor, which could be a piezo electric or piezo resistive motility sensor, or an accelerometer. In the alternative, a acoustic sensor, such as a microphone, may be used to sense the patient swallowing by picking up the sound generated by the patient swallowing. In the alternative, an optical sensor may be used for sensing the opacity alteration over the esophagus as food passes. A strain sensor could also be used for sensing the expansion of the esophagus as food passes. The features and functions of sensors sensing the patient swallowing is further described with reference to FIG. 16. The controller may comprise at least one unit having a sleep mode and an active mode, and the unit consumes less energy in the sleep mode than in the active mode. The unit is configured to switch from the sleep mode to the active mode on the basis of at least one signal from the acoustic sensor. The unit could for example be a DSP (Digital Signal Processor), another type of processor or a wake-up circuit of the controller, which in turn activates the functions of the controller. The unit may be configured to switch from the sleep mode to the active mode on the basis of a signal from the acoustic sensor related to the patient swallowing a number of times and/or on the basis of a signal from the acoustic sensor related to the patient swallowing a number of times during a time period. The number of times the patient swallows and the time could be counted/measured and compared with a pre-set or moving threshold value. The controller could further comprise at least one filtering unit configured to filter acoustic signals related to at least one of: speech, the swallowing of saliva and chewing. The filter could be a digital filter implemented as hardware or software in the controller and could have the filter characteristics of a high, low or bandpass filter.

A local temperature, to avoid local excessive heating which may damage tissue of the patient.

A systemic temperature, to avoid systemic excessive heating which may cause fever and affect the overall wellbeing of the patient.

Blood saturation/oxygenation, or a parameter related to an ischemia marker such as lactate, to control and/or avoid that the flow of blood to some tissue portion is hampered by the implantation or operation of the medical device 10. Hampered blood flow may lead to tissue damage and in the worst cases to tissue necrosis.

Blood pressure, which may be an indication that the strain created by the medical device is in some way damaging to the overall wellbeing of the patient. Increased blood pressure may be used for triggering an alarm function for alerting the patient or physician.

pH, for determining the acidity of the stomach, which could be an indicator of the function of the digestive system and/or of the frequency of ingestion. The pH may be used for controlling the medical device 10 on the basis of the patient eating or drinking.

The controller 300 may comprise a transmitter configured to transmit signals containing information to a receiver located exterior to the body of the patient. The transmitter could be a wireless transmitter configured to communicate with a wireless receiver located external to the body of the patient. In the embodiments shown in FIGS. 1a-1c, the wireless transmitter of the controller 300 is configured to transmit information based on sensor input to a source external to the body of the patient.

In some embodiments, the medical device further comprises a capacitor connected to the implantable energy storage unit 40 and connected to the operation device 100. The capacitor is configured to be charged by the implantable energy storage unit 40 and to provide the operation device with electrical power. The capacitor can compensate for situations in which the discharging of energy from the implantable energy storage unit 40 during startup of the operation device 100 is slower than the energy needed for startup of the operation device. That is, there is a difference between the energy needed by the operation device and the energy the implantable energy storage unit 40 is capable of providing without damaging the implantable energy storage unit 40. In other words, a maximum energy consumption of the energy consuming part may be higher than the maximum energy capable of being delivered by the implantable energy storage unit 40 without causing damage to the implantable energy storage unit, and the capacitor may be adapted to deliver an energy burst corresponding to difference between the required energy consumption and the maximum energy capable of being delivered by the implantable energy storage unit 40. The implantable energy storage unit 40 may be configured to store a substantially larger amount of energy than the capacitor but may be slower to charge. The capacitor could be a start capacitor, a run capacitor, a dual run capacitor or a supercapacitor.

The medical device 10 may further comprise a sensation generator adapted to generate a sensation detectable by a sense of the patient. The sensation generator could for example be a vibrator or a heat generating unit. The sensation generator could be used to provide feedback to the patient for communicating a functional status of the medical device 10 or a physiological status of the patient. Such communication could for example include information on the status of the implantable energy storage unit 40 of the medical device or alert the patient about the operation of the medical device or of an error related to the medical device.

The controller 300 may further comprise a receiver for receiving patient generated control signals from a unit located external to the body of the patient. The receiver could be a wireless receiver configured to communicate with a transmitter located external to the body of the patient. The controller 300 may be configured to control the operation device 100 on the basis of the received patient generated control signal. The control signal could for example being that the patient indicates to the medical device 10 that the patient has finished a portion of food which causes the medical device 10 to operate to stretch the stomach wall SW of the patient such that satiety is created which reduces the patients willing to eat further. In the alternative, the controller 300 may be configured to control the operation device 100 on the basis of a signal related to a lapsed time or a time of day such that satiety can be created with certain intervals or during specific periods of the day. The controller 300 could further be configured to receive a signal from a sensor external to the body of the patient and use such signal for controlling the operation of the medical device 10. The sensor external to the body of the patient could be a sensor could be a sensor measuring a parameter related to the patient eating to create input for the control of the medical device 10. Such a parameter could be related to body temperature, blood pressure or the glucose level of the blood. In the alternative, the sensor could be a sensor sensing a parameter related to the external environment, such as the atmospheric pressure, which could affect the pressures in the medical device 10.

Further features and function which may be implemented in the controller 300 is further described with reference to FIGS. 65a-65f.

FIG. 1b′ shows the medical device 10 in a state in which the second member 101b has been pivoted downwards such that the distance between the upper portion of the second member, invaginated by the tissue T1″, T2″ of the stomach wall, is moved away from the upper portion of the first member, invaginated by the tissue T1′, T2′ of the stomach wall, such that the stomach wall between these portions is stretched.

FIG. 1c shows the medical device 10 in a state in which the first and second members 101a, 101b have been folded 90 degrees in a direction substantially perpendicular to the direction in which the second member 101b is displaced in relation to the first member 101a. The folding of the first and second members 101a, 101b reduces the width of the medical device 10 from a first width W′ to a second width W″ which facilitates insertion of the medical device 10 into the body of the patient. The folding of the first and second members 101a, 101b enables the insertion of the medical device 10 though a trocar or a gastroscope which enables minimally invasive placement of the medical device. In the embodiment of FIG. 1c, the folding is enabled by the hinges 26a′,26a″. In the embodiment shown in FIG. 1a-1c the folding of the first and second members 101a, 101b reduces the width of the medical device 10 with more than 50%, i.e. W″ is less than 0.5 times the width of W′. In alternative embodiments the width W″ may be less than 0,7 times the width W′, or the width W″ may be less than 0,6 times the width W′, or the width W″ may be less than 0,4 times the width W′.

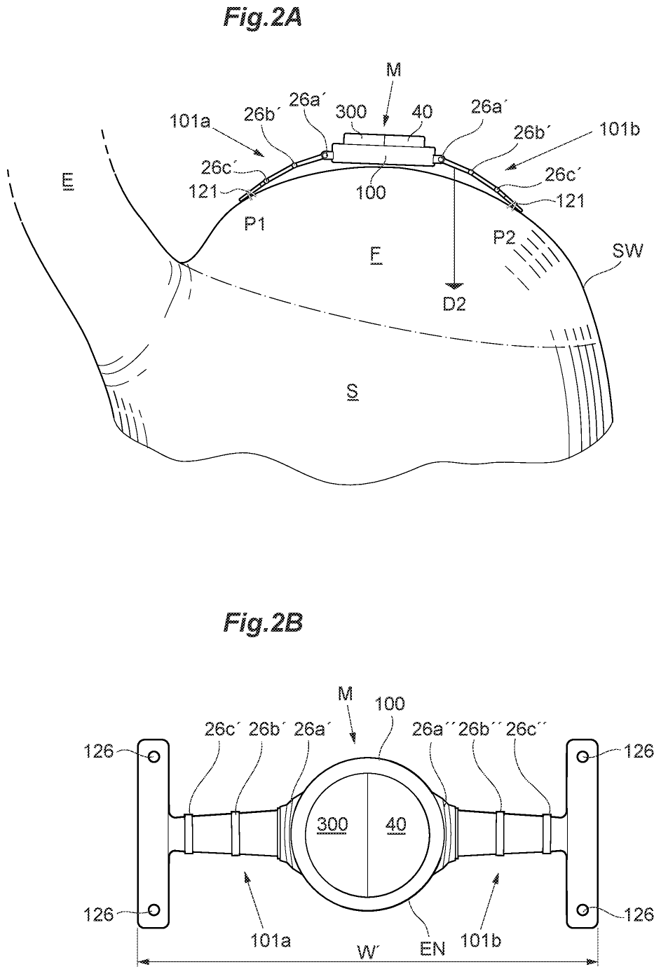

FIGS. 2a and 2b shows an embodiment of the medical device 10 similar to the embodiment shown with reference to FIGS. 1a-1c, the difference being that in the embodiment of FIGS. 2a and 2b the first and second members 101a, 101b comprises a fixation portion comprising a through hole 126 enabling the fixation of the first and second member to the tissue of the stomach wall SW using sutures or staplers. As such, the first and second members 101a, 101b can be fixated to the stomach wall SW without invagination of the portion of the members 101a, 101b. FIG. 2a shows the medical device 10 when the first and second members 101a, 101b have been fixated to the tissue of the stomach wall SW using sutures 121. I alternative embodiments, not shown, the through holes could be replaced by recesses in the first and second members 101a, 101b enabling fixation of the first and second members 101a, 101b by suturing over the first and second members 101a, 101b.

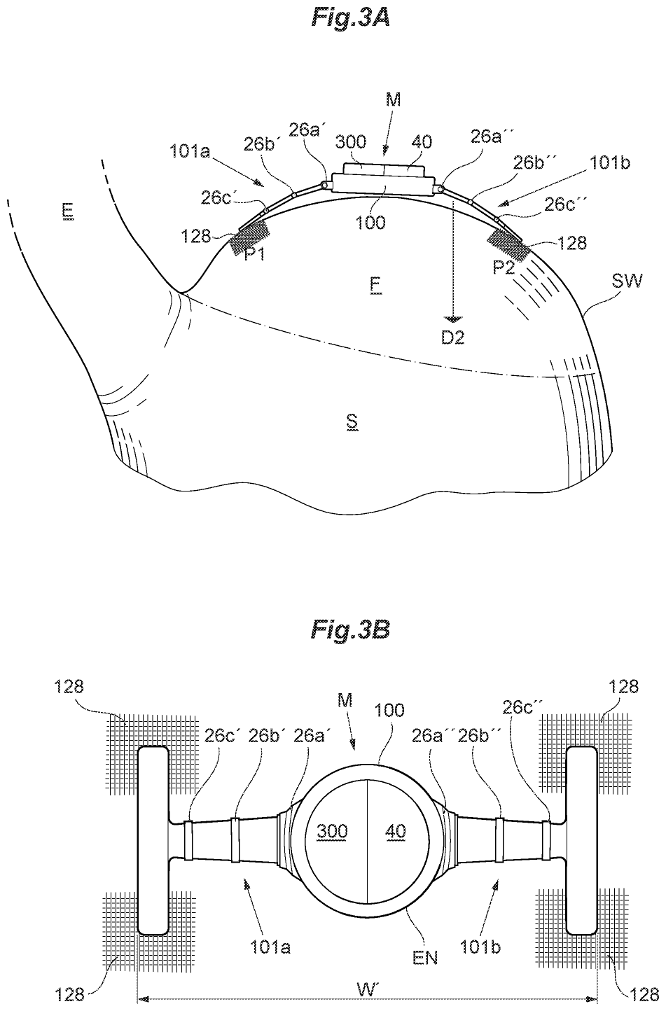

FIGS. 3a and 3b shows an embodiment of the medical device 10 similar to the embodiments shown with reference to FIGS. 1a-2b, the difference being that in the embodiment of FIGS. 3a and 3b the first and second members 101a, 101b comprises tissue growth promoting structure in the form of a mesh 128 configured to be fixated to the tissue of the stomach wall SW by means of fibrotic tissue growing into the mesh. By means of the tissue growth promoting structure the first and second members 101a, 101b can be fixated to the stomach wall SW without invagination of the portion of the members 101a, 101b. FIG. 3a shows the medical device 10 when the first and second members 101a, 101b have been fixated to the tissue of the stomach wall SW using the tissue growth promoting structure.

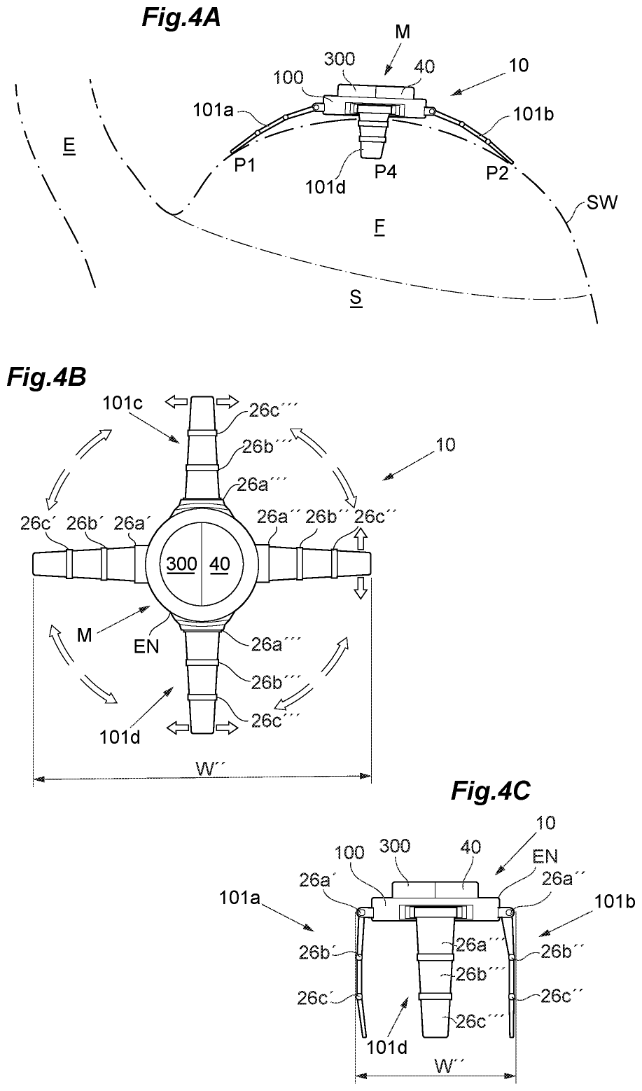

FIGS. 4a-4c shows an embodiment of the medical device 10 similar to that of FIGS. 1a-3b. However, in the embodiment shown in FIGS. 4a-4c the medical device further comprises third member 101c configured to be fixated to a third portion of the stomach wall, and a fourth member 101d configured to be fixated to a fourth portion P4 of the stomach wall SW. The operation device 100 is further configured for operating the third member 101c to displace the third member 101c relative to the first and second members 101a, 101b, for stretching the stomach wall between the third portion and the first and second portions P1, P2 respectively. The operation device 100 is further configured for operating the fourth member 101d to displace the fourth member 101d relative to the first and second members 101a, 101b, for stretching the stomach wall between the fourth portion P4 and the first and second portions P1, P2 respectively.

In the embodiment shown in FIGS. 4a-4c only the first member is inoperably fixated to the main portion M, whereas the second, third and fourth members 101b, 101c, 101d are operably fixated to the main portion M. However, in alternative embodiments, the first and second members 101a, 101b may be inoperably fixated to the main portion M and the third and fourth members 101b, 101c, 101d may be operably fixated to the main portion M.

The first, second, third and fourth members 101a, 101b, 101c, 101d may be fixated to the stomach wall SW by means of invagination, such as described in relation to FIGS. 1a-1c, or by means of sutures or staplers, such as described in relation to FIGS. 2a-2b, or by means of tissue growth promoting structure, such as described in relation to FIGS. 3a-3b.

Similarly to what is described in relation to the embodiments shown in FIGS. 1a-3b, the first, second, third and fourth members 101a, 101b, 101c, 101d are hinged for enabling pivotal movement. In the embodiment shown in FIGS. 4a-4c the first, second, third and fourth members 101a, 101b, 101c, 101d are hinged at a first, second and third pivotal points 26a′,26b′,26c′, 26a″,26b″,26c″,26a′″,26b″′,26c″′,26a″″,26b″″,26c″″ which enables the first, second, third and fourth members 101a, 101b, 101c, 101d to adapt to the curvature of the stomach S following the curvature of the fundus F part of the stomach wall SW.

Similarly to what is described in relation to the embodiments shown in FIGS. 1a-3b, the main portion M comprises an enclosure EN enclosing the operation device 100. The enclosure EN is flexible to enable the displacement of the second, third and fourth members 101b, 101c, 101d for stretching the stomach wall SW.

FIG. 4c shows the medical device 10 in a state in which the first, second, third and fourth members 101a, 101b, 101c, 101d have s been folded 90 degrees in a direction substantially perpendicular to the direction in which the second member 101b is displaced in relation to the first member 101a. The folding of the first, second, third and fourth members 101a, 101b, 101c, 101d reduces the width of the medical device 10 from a first width W′ to a second width W″ which facilitates insertion of the medical device 10 into the body of the patient. The folding of the first, second, third and fourth members 101a, 101b, 101c, 101d enables the insertion of the medical device 10 though a trocar or a gastroscope which enables minimally invasive placement of the medical device 10. In the embodiment of FIG. 4c, the folding is enabled by the hinges 26a′,26a″,26a″′,26a″″. In the embodiment shown in FIG. 1a-1c the folding of the first, second, third and fourth members 101a, 101b, 101c, 101d reduces the width of the medical device 10 with more than 50%, i.e. W″ is less than 0.5 times the width of W′. In alternative embodiments the width W″ may be less than 0,7 times the width W′, or the width W″ may be less than 0,6 times the width W′, or the width W″ may be less than 0,4 times the width W′.

A method of implanting the medical device 10 for actively stretching a stomach wall of a patient for creating a sensation of satiety, according to any one of the embodiments herein is further provided.

The method comprises the steps of making an incision in the abdomen of the patient, for accessing the area of the stomach S. The incision may be a single incision for implanting the implantable medical device for actively stretching a stomach wall using open surgical techniques or may be a plurality of incisions for implanting the implantable medical device 10 for actively stretching a stomach wall using minimally invasive, endoscopic techniques, or a combination of open and endoscopic techniques. In alternative embodiments the incision could be a single incision for the insertion of a laparoscopic port with multiple entry port enabling a laparoscopic procedure with a single incision (SILS). For enabling optical inspection in a minimally invasive procedure a cavity within the body needs to be created, which is typically done by means of pressurized CO2 gas being introduced through a trocar placed in an incision.

The method further comprises the step of dissecting a portion of the stomach S for preparing the portion of the stomach, preferably the fundus F part of the stomach for the placement and fixation of an implantable medical device 10 for actively stretching a stomach wall.

The method further comprises the step of inserting an implantable medical device 10 for actively stretching a stomach wall into the body of the patient. The method may be commenced as a minimally invasive procedure (such as Laparoscopic, SILS, NOTES etc.) and continued as open surgery when the implantable medical device 10 for actively stretching a stomach wall should be inserted. The procedure could also be performed as a hand assisted minimally invasive procedure in which the surgeon can insert a hand through a small incision in the abdomen. Hand assisted surgery has the benefit of providing sensory perception and the possibility to guide the surgical instruments whilst maintaining the possibility of visually observing the entire procedure on a TV screen.

The medical device 10, as described in many of the embodiments herein, comprises at least a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions. In some embodiments, the method could comprise using the flexibility of at least one of the first and second member for adapting the medical device to the curvature of the stomach of the patient, and fixating the first member to a first portion of the stomach wall, and fixating the second member to a second portion of the stomach wall.

The method may further comprise the step of fixating the third member to a third portion of the stomach wall, and fixating the fourth member to a fourth portion of the stomach wall.

In the embodiment shown in FIG. 4c the first, second, third and fourth members 101a-101d are connected to a main portion M, and the method comprises using flexibility of at least one of the first, second, third and fourth members 101a-101d for adapting the medical device 10 to the curvature of the stomach S of the patient by pivoting at least one of the first, second, third and fourth members relative to the main portion M.

At least one of the steps of fixating the first member 101a to a first portion of the stomach wall, and fixating the second member 101b to a second portion of the stomach wall may comprise at least partially invaginated at least one of the first and second member by the tissue of the stomach wall SW. In the alternative, or as a complement, fixating the first member to a first portion of the stomach wall, and fixating the second member to a second portion of the stomach wall may comprise fixating at least one of the first and second member to tissue of the stomach wall using at least one of sutures, staplers or tissue growth promoting structure.

The surgical method may further comprise placing at least one sensor in the area of the esophagus of the patient for sensing a parameter related to the patient swallowing. The step of placing at least one sensor in the area of the esophagus of the patient for sensing a parameter related to the patient swallowing may comprise placing at least one of: a motility sensor, a acoustic sensor, an optical sensor or a strain sensor.

The method may further comprise placing at least one sensor in the area of the stomach of the patient for sensing a parameter related to the patient eating. The step of placing at least one sensor in the area of the stomach of the patient for sensing a parameter related to the patient eating may comprise placing at least one of temperature sensor, blood saturation or oxygenation sensor, a blood pressure sensor, a sensor configured to sense a parameter related to an ischemia marker, or a pH sensor. The step of sensing pH comprises sensing the acidity in the stomach.

The method may further comprise placing an electrode arrangement configured to be arranged between the medical device and the stomach to engage and electrically stimulate muscle tissue of the stomach to exercise the muscle tissue to improve the conditions for long term implantation of the medical device.

The method may further comprising placing an implantable remote unit (such as further described with reference to FIGS. 33-53b) connected to the medical device in the body of the patient.

The step of placing an implantable remote unit connected to the medical device in the body of the patient may comprise placing an implantable remote unit comprising: at least a portion of the operation device and/or an energy storage unit and/or a wireless energy receiver and/or a wireless transceiver for wireless communication.

The flexibility of at least one of the first and second member could also be used for facilitating insertion of the medical implant into the body of the patient. The flexibility can be used for compressing the medical device such that it can be inserted through an incision in the skin of the patient or through a cannula of a trocar and into the body of the patient.

The flexibility can then enable at least the first and second member to expand after the medical device has been inserted into the body of the patient. The flexibility can be achieved by pivoting at least one of the first, second, third and fourth members relative to the main portion.

After implantation, the implantable medical device could require calibration, and the surgical method may comprise the necessary steps for calibrating the medical device for actively stretching a stomach wall of a patient for creating a sensation of satiety. A calibration method may comprise receiving, at the controller, a first input signal comprising at least one of: a sensor input signal related to a physical parameter of the medical device, and an input signal from a source external to the body of the patient, and controlling, by the controller, the operation device to adjust the stretching of the stomach wall, in response to the first input signal, and receiving, at the transceiver, a second input signal from the source external to the patient, and controlling, by the controller, the operation device to further adjust the stretching of the stomach wall, in response to the second input signal.

The sensor input signal could be related to a physical parameter of the medical device is a sensor input related to at least one of: energy consumption, position of at least one of the first member, the second member and the operation device, strain on at least one of the first member, the second member and the operation device, and speed of the operation device.

The input signal from a source external to the body of the patient could be an input signal generated by the patient, which could be a feeling of satiety experienced by the patient, a feeling of pain experienced by the patient, and the patient ingesting something.

The step of adjusting the stretching of the stomach wall could comprise adjusting one of: the force with which the stomach wall is stretched, the speed with which the stomach wall is stretched, the duration of the stretching of the stomach wall.

The step of inserting an implantable medical device 10 for actively stretching a stomach wall into the body of the patient may comprise the step of inserting an implantable controller 300 into the body of the patient, for controlling the implantable medical device 10. The step of inserting an implantable controller 300 may comprise fixating the implantable controller to tissue or bone in the body of the patient. Functions and features of the implantable controller 300 is further described throughout this application and in particular with reference to FIGS. 65a-65f.

The surgical method may, in hydraulic embodiments, further comprise the step of calibrating the fluid level in the implantable medical device through injection or retraction of fluid via the implantable injection port. Calibration of fluid levels can be done at routine check-up or in response to the medical device not functioning optimally or in response to the implantable medical device transmitting a signal indicating that the fluid level needs to be calibrated. The need for calibration can be based on leakage or diffusion of fluid from the implantable medical device.

The method may further comprise the step of calibrating the pressure or strain exerted by the implantable medical device on the stomach wall, which may comprise calibrating the pressure in the implantable medical device through the measurement of the pressure in the implantable medical device, e.g. by means of a pressure sensor in direct or indirect contact with the fluid in the implantable medical device or by the measurement of strain in the main portion or the members. The pressure/strain could be calibrated relative to the blood pressure of the patient. This could be used to make sure that the tissue of the stomach wall is not constricted such that the blood flow in the tissue is adversely affected or hampered. The pressure/strain exerted on the stomach wall could be calibrated relative to the systolic blood pressure of the patient, such that the pressure does not exceed the systolic blood pressure, to allow blood to be pressed into the tissue during the systolic cardiac phase. In the alternative, the pressure/strain could be calibrated relative to the diastolic blood pressure of the patient, such that the pressure does not exceed the diastolic blood pressure, to allow normal circulation through the tissue of the stomach wall.

The method may further comprise the step of calibrating the speed with which the implantable medical device should stretch the stomach wall. This could allow the patient to provide feedback to the device with regards to the feeling of satiety such that the device functions in an optimal way.

In embodiment in which electrical stimulation is used to counteract the effects of long-term implantation, the method may further comprise the step of calibrating the electrical stimulation of the tissue on the basis of a physiological marker, such as an ischemia marker, or on the basis of input from the patient e.g. related to a sensory response induced by the electrical stimulation, such as pain related to the electrical stimulation.

The method may further comprise performing tests related to the function of the implantable medical device. These tests may be performed during the surgical procedure or in closely after the surgical procedure. The testing could be testing a feedback function by providing sensory feedback to the patient, which could be sensory feedback in the form of vibrations created by the motor of the implantable medical device, or created by a separate vibrator. Sensory feedback could in the alternative be created in the form of electrical stimulation.

The method may comprise a step of testing a post-operative mode for enabling healing of the stomach wall and the surrounding tissue after implantation. It may be the case that the tissue surrounding the device needs to heal before the device may be used to stretch stomach wall. It may also be the case that the device needs to be fixated by the growth of fibrotic tissue in the invagination or into a fixating structure for the fixation of the members, which may be tested in a test of a post-operative mode for enabling growth of fibrotic tissue.

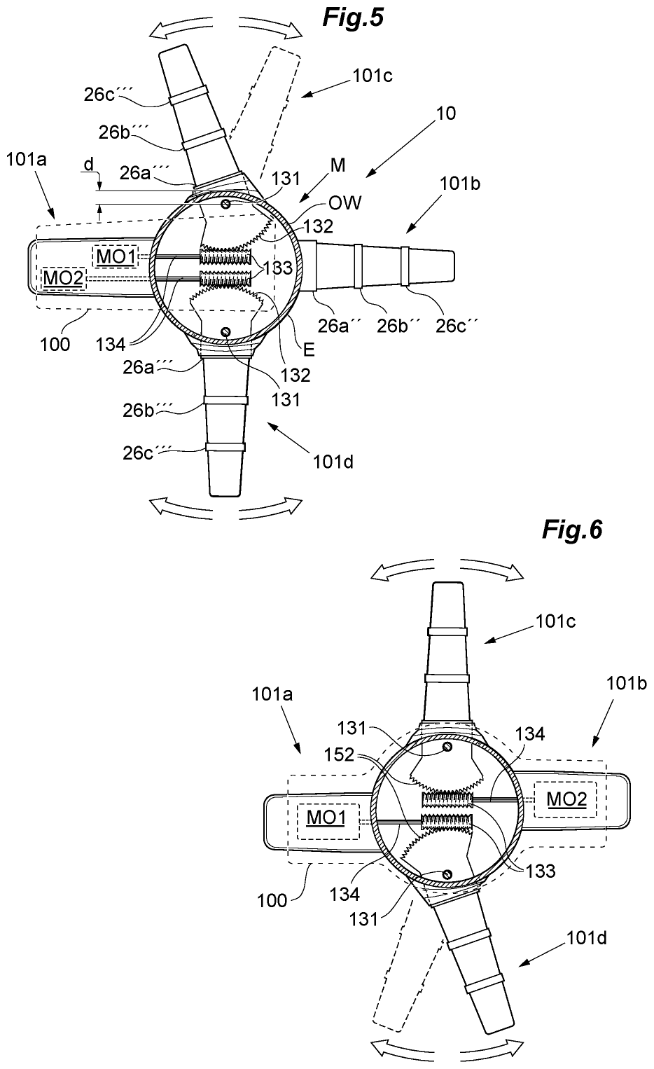

FIG. 5 shows an embodiment of the medical device 10 similar to that of FIGS. 4a-4c. In the embodiment shown in FIG. 5 the first and second member 101a, 101b are inoperably fixated to the main portion M of the medical device 10, while the third and fourth member 101c, 101d are operably fixated to the main portion M of the medical device 10. The third and fourth members 101c, 101d are operably fixated to the main portion M at a pivot point 131 such that the third and fourth members 101c, 101d can be displaced in relation to the first and second members 101a, 101b. The operation device 100, being a mechanical operation device is placed partially in the main portion M and partially in the first member 101a. The operation device comprises a first portion configured to operate the third member 101c. The first portion of the operation device 100 comprises a first motor MO1 placed in the first member 101a.

The first motor MO1 is connected to a shaft 134 which in turn is connected to a worm 133 which engages a portion of a worm wheel 132 integrated in the third member 101c for creating a worm drive placed in the main portion M for displacing the third member 101c, such that the third member 101c rotates around the pivot point 131. The second portion of the operation device 100 comprises a second motor MO2 placed in the first member 101a. The second motor MO2 is connected to a shaft 134 which in turn is connected to a transmission in the form of a worm 133 which engages a portion of a worm wheel 132 integrated in the fourth member 101d for creating a worm drive placed in the main portion M for displacing the fourth member 101d, such that the fourth member 101d rotates around the pivot point 131. As such, the transmission in the form of a worm drive transforms the rotating mechanical force from the electrical motors MO1, MO2 into a liner mechanical force for stretching the stomach wall. The motors MO1 and MO2 are in the embodiment shown in FIG. 5 electrical motors with integrated gearboxes to lower the speed of the electrical motors to an RPM suitable for operating the worm drive. The motor could for example be an implantable brushless DC motor with integrated gear box, such as the motors provided by Maxon group or Dr. Fritz Faulhaber.

The points of pivot 131,132 are according to the embodiment shown in FIG. 5 placed very close to the outer wall of the enclosure EN enclosing the main portion M. Placing the points of pivot close to the outer wall of the enclosure EN means that the movement of the members 101c, 101d, in the area of the outer wall of the enclosure EN, in relation to the outer wall of the enclosure EN is small, which means that when the medical device has been encapsulated by fibrotic tissue, the members 101c, 101d will still be able to pivot in relation to the main portion M, as the bending of the fibrotic tissue at the intersection between the displaceable members 101c, 101d and the main portion M will be small.

In the embodiment shown in FIG. 5, the point of pivot is placed at a distance d from an outer wall of the main portion M. The distance d being in the interval 1 mm-10 mm, or in the interval 1 mm-7 mm, or in the distance 2 mm-6 mm, or in the interval 3 mm-5 mm. The distance d being in the interval 2-5 times the thickness of the outer wall OW of the enclosure EN, or in the interval from 2-4 times the thickness of the outer wall OW of the enclosure.

When implanted, the first member 101a is fixated to a first portion of the stomach wall and the second member 101b is fixated to a second portion of the stomach wall and the third member 101c is fixated to a third portion of the stomach wall and the fourth member 101d is fixated to a fourth portion of the stomach wall. The first motor MO1 rotates in a first direction and thereby operates the third member 101c via the worm drive 133,132 such that the third member 101c is displaced in a direction towards the first member 101a, which increases the distance between the third member 101c and the second member 101b, which thereby increases the distance between the third portion of the stomach wall, to which the third member 101c is fixated, and the second portion of the stomach wall, to which the second member 101b is fixated. The increasing distance between the third and second portions stretches the tissue of the stomach wall between the third and second portions which activates the stretch receptors in the stomach wall creating a feeling of satiety in the patient. The first motor MO1 next rotates in a second direction and thereby operates the third member 101c via the worm drive 133,132 such that the third member 101c is displaced in a direction towards the second member 101b, which increases the distance between the third member 101c and the first member 101a, which thereby increases the distance between the third portion of the stomach wall, to which the third member 101c is fixated, and the first portion of the stomach wall, to which the first member 101a is fixated. The increasing distance between the third and first portions stretches the tissue of the stomach wall between the third and first portions which activates the stretch receptors in the stomach wall creating a feeling of satiety in the patient. Next, the second motor MO2 rotates in a first direction and thereby operates the fourth member 101d via the worm drive 133,132 such that the fourth member 101d is displaced in a direction towards the first member 101a, which increases the distance between the fourth member 101d and the second member 101b, which thereby increases the distance between the fourth portion of the stomach wall, to which the fourth member 101d is fixated, and the second portion of the stomach wall, to which the second member 101b is fixated. The increasing distance between the fourth and second portions stretches the tissue of the stomach wall between the fourth and second portions which activates the stretch receptors in the stomach wall creating a feeling of satiety in the patient. The second motor MO2 next rotates in a second direction and thereby operates the fourth member 101d via the worm drive 133,132 such that the fourth member 101d is displaced in a direction towards the second member 101b, which increases the distance between the fourth member 101d and the first member 101a, which thereby increases the distance between the fourth portion of the stomach wall, to which the fourth member 101d is fixated, and the first portion of the stomach wall, to which the first member 101a is fixated. The increasing distance between the fourth and first portions stretches the tissue of the stomach wall between the fourth and first portions which activates the stretch receptors in the stomach wall creating a feeling of satiety in the patient.

The first, second, third and fourth members 101a, 101b, 101c, 101d of the embodiment of FIG. 5 may be fixated to the stomach wall SW by means of invagination, such as described in relation to FIGS. 1a-1c, or by means of sutures or staplers, such as described in relation to FIGS. 2a-2b, or by means of tissue growth promoting structure, such as described in relation to FIGS. 3a-3b.

Similarly to what is described in relation to the embodiments shown in FIGS. 1a-3b, the second, third and fourth members 101b, 101c, 101d are hinged for enabling pivotal movement in a direction substantially perpendicular to the displacement directions of the third and fourth members 101c, 101d. The second, third and fourth members 101b, 101c, 101d are hinged at a first, second and third pivotal points 26a″,26b″,26c″,26a″′,26b″′,26c″′,26a″″, 26b″″,26c″″ which enables the second, third and fourth members 101b, 101c, 101d to adapt to the curvature of the stomach S following the curvature of the fundus F part of the stomach wall SW.

Similarly to what is described in relation to the embodiments shown in FIGS. 1a-3b, the main portion M comprises an enclosure EN enclosing a portion of the operation device 100. The enclosure EN is flexible to enable the displacement of the third and fourth members 101c, 101d for stretching the stomach wall SW.

The operation device of the embodiment of FIG. 5 may be controlled by a controller and powered by an implantable energy storage unit. The controller and/or the energy storage unit could be placed locally in the medical device 10, in direct connection with the operation device 100, such as shown in the embodiments of FIGS. 1a-4c. The controller and/or the energy storage unit could in alternative embodiments be placed remotely in a remote unit such as further described with reference to FIGS. 33-53b.

FIG. 6 shows an embodiment very similar to the embodiment shown in FIG. 5. The only difference is that the first motor MO1 is placed in the first member 101a and the second motor MO2 is placed in the second member 101b. As both the first and second members 101a, 101b now comprises a portion of the operation device 100 in the form of the motors, the hinges have been omitted from the first and second members 101a, 101b and as such, it is only the third and fourth members having hinges enabling the adaptation to the curvature of the stomach or the folding of the members for the insertion of the medical device during a minimally invasive procedure.

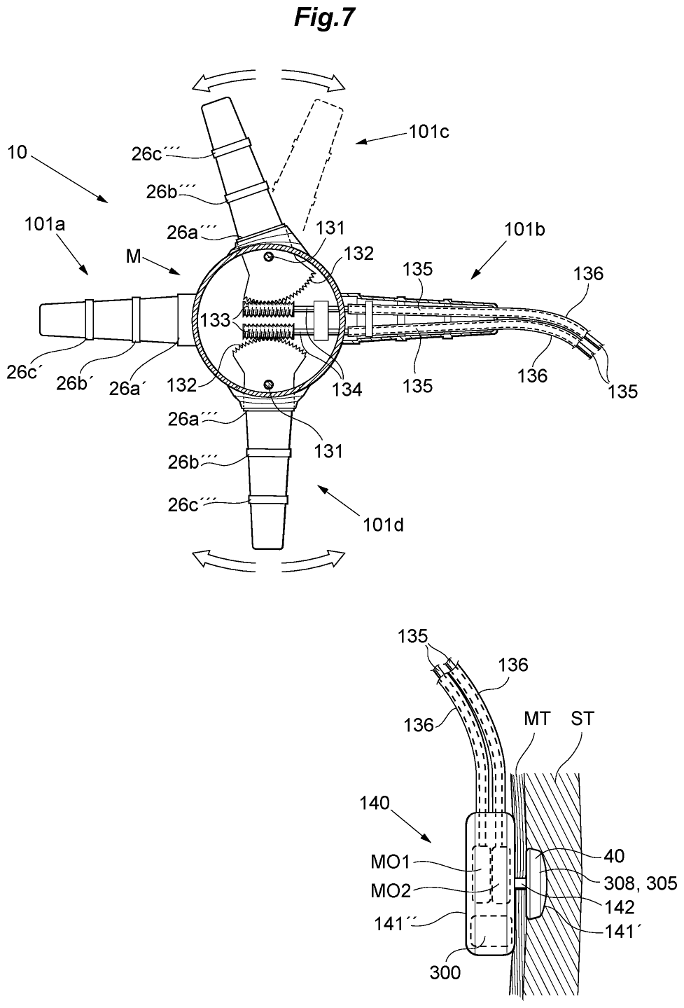

FIG. 7 shows an embodiment of the medical device 10 very similar to the embodiment shown in FIG. 5. The difference between the embodiment of FIG. 7 and the embodiment of FIG. 5 is that in the embodiment of FIG. 7, the motors MO1 and MO2 are placed in a remote unit 140. Just as in the embodiment described with reference to FIG. 5, the third and fourth members 101c, 101d are operable by transmission in the form of a worm drives which together with the third and fourth members 101c, 101d transforms the received rotating mechanical force into a liner mechanical force. Each worm drive comprises a portion of a worm wheel 132 integrated with the third and fourth member 101c, 101d, respectively. The worm wheel portions 132 are engaged and operated by worms 133 each of which being fixated to rotatable shafts 134. The rotatable shafts 134 transitions into a flexible shaft or rotating Bowden cable 135. The flexible shafts 135 transmits rotary motion from the motors MO1, MO2 in the remote unit 140 to the portion of the operation device located in the main portion M of the medical device 10. The flexible shaft 135 comprises wire made up of a plurality of strands making the wire flexible but with relatively high torsional stiffness. The wire is enclosed by a flexible protective cover 136 which encloses and protects the flexible shaft 135 from the environment in the body. The protective cover 136 comprises an inner sleeve for reducing the friction between the rotating wire and the protective cover 136, next the protective cover 136 comprises a structure providing support and protection and being configured to manage some level of external compression without that affecting the movement of the flexible shaft 135. The outermost layer of the protective cover 136 is a protective cover for sealing the flexible shaft 135 against the environment in the body of the patient. The protective cover comprises a biocompatible flexible material, such as a biocompatible flexible polymer material, such as a silicone-based material, or a polyurethane-based material. In the embodiment shown in FIG. 7, the proximal portion of the two flexible wires 134, being the portion most proximal to the main portion M, for operating the third and fourth members 101c, 101d, respectively, passes through the second member 101b, of the medical device 10. The second member 101b being inoperably fixated to the main portion M and forms a receiving portion for receiving the mechanical force created by the motors MO1, MO2.

The distal portions of the two flexible shafts 135, being the portions most proximal to the remote unit 140, are mechanically connected to the first and second motor MO1, MO2, respectively, such that rotating mechanical force created by the motors MO1, MO2 is transferred to the main portion by means of the flexible shafts 135. In the embodiment shown in FIG. 7, the remote unit 140 comprises a first portion 141′, a second portion 141″, and a connecting portion 142, mechanically connecting the first and second portons 141′,141″. The second portion 141″ is in the embodiment shown in FIG. 7 placed on the inside of muscular tissue MT of the abdominal wall of the patient, whereas the first portion 141′ is place on the outside of the muscular tissue MT of the abdominal wall, in the subcutaneous tissue ST. As such, the connecting portion 142 travels through a created hole or natural orifice of the muscular tissue MT. A cross-sectional area of the connecting portion 142, in a plane in the extension of the muscular tissue MT is smaller than a cross-sectional area of the first and second portions 141′,141″, parallel to the cross-sectional area of the connecting portion 142. The cross-sectional areas of the first and second portions 141′,141″ are also larger than the created hole or natural orifice though which the connecting portion 142 is placed. As such, the first and second portions 141′,141″ are unable to pass through the created hole or natural orifice and is as such fixated to the muscular tissue MT of the abdominal wall. In the embodiment shown in FIG. 7, the connecting portion 142, is a connecting portion having a circular cross-section and an axial direction extending from the first portion 141′ to the second portion 141″. The plane in the extension of the muscular tissue MT, is in the embodiment of FIG. 7 perpendicular to the axial direction of the connecting portion 142 extending from the first portion 141′ to the second portion 141″.

In the embodiment of FIG. 7, the controller 300 is placed in the second portion 141″, and the implantable energy storage unit 40 is placed in the second portion 141″. In the embodiment of FIG. 7, the controller 300 and the implantable energy storage unit 40 are electrically connected by means of a lead running in the connecting portion 142, such that electrical energy and communication can be transferred from the second 141″ to the first portion 141′, and vice versa. In the embodiment of FIG. 7, the first portion 141′ further comprises a wireless energy receiver 305 for receiving wireless energy for charging the implantable energy storage unit 40 and/or for powering the medical device 10, and a transceiver 308 for receiving and/or transmitting wireless signals to/from the outside the body. Features and functions of the remote unit 140 are further described with reference to FIGS. 33-53b. The controller 300 and energy storage unit 40 may have the same features and functions as the controller 300 and energy storage unit 40 when described as integrated in the main portion M. Further features and functions of the controller 300 and the implantable energy storage unit 40 are further described with reference to FIGS. 65a-65f.

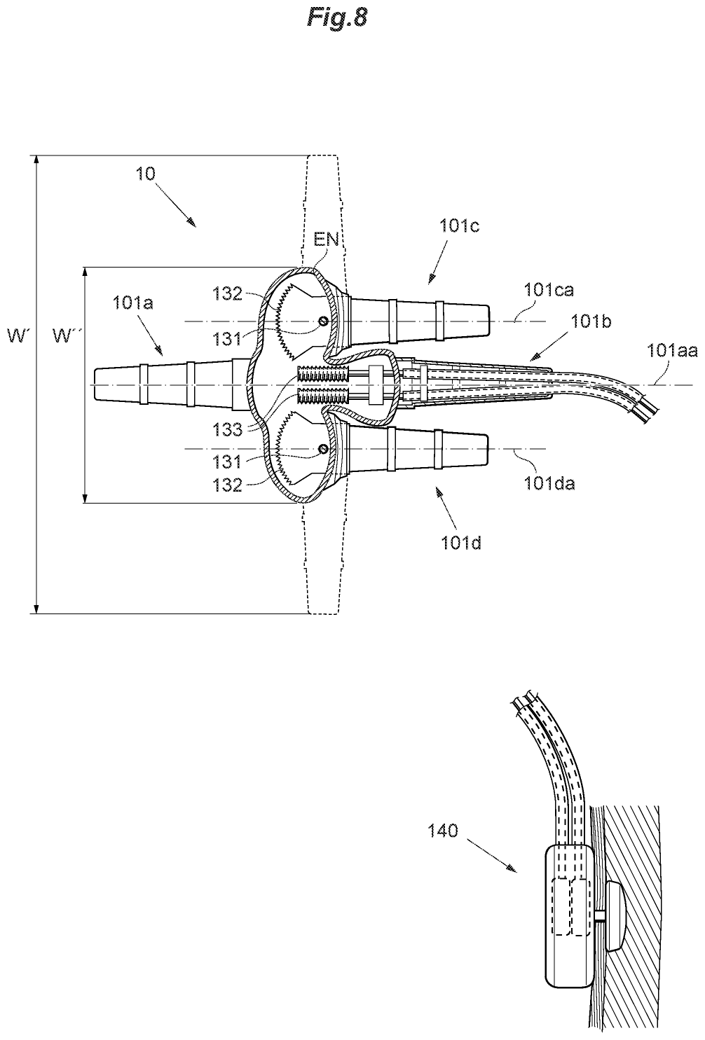

FIG. 8 shows the medical device 10 of the embodiment shown in FIG. 7, when the third and fourth members 101c, 101d have been folded around the pivotal points 131 for facilitating the insertion of the medical device 10 into the body of the patient. By folding the third and fourth members 101c, 101d, the medical device 10 can be inserted into the body of a patient through a trocar or gastroscope. In the embodiment shown in FIG. 8, the third and fourth members 101c, 101d have been folded such that a length axis 101ca running centrally in the third member 101c is placed parallel to a length axis 101aa running centrally in the first and second members 101a, 101b, and a length axis 101da running centrally in the fourth member 101c is placed parallel to a length axis 101aa running centrally in the first and second members 101a, 101b. In the embodiment of FIG. 8, it can be seen that the enclosure EN is flexible enough to enable the folding of the third and fourth members. In the folded state, the worm wheels 132 are disconnected from the worms 133. After insertion of the medical device 10 into the body of the patient, the third and fourth members 101c, 101d are rotated such that the worm wheels 132 and worms 133 are connected enabling the third and fourth members 101c, 101d to be operated by the worm drive. The folding of the third and fourth members 101c, 101d reduces the width of the medical device 10 from a first width W′ to a second width W″ which facilitates insertion of the medical device 10 into the body of the patient. In the embodiment of FIG. 8, the folding of the third and fourth members 101c, 101d reduces the width of the medical device 10 with more than 30%, i.e. W″ is less than 0,7 times the width of W′.

In alternative embodiments the width W″ may be less than 0.5 times the width W′, or the width W″ may be less than 0,6 times the width W′, or the width W″ may be less than 0,8 times the width W′.

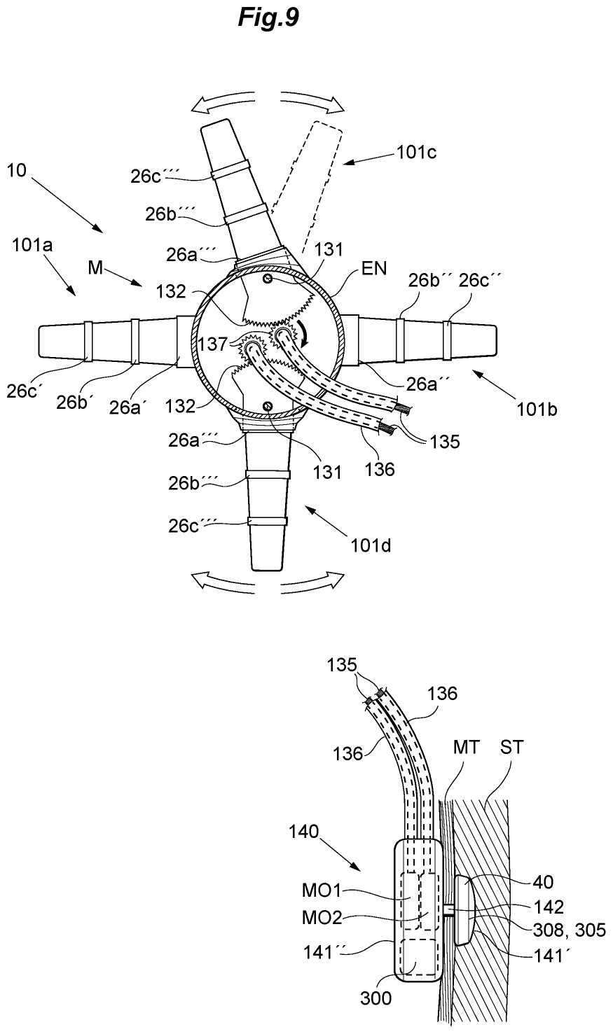

FIG. 9 shows an embodiment of the medical device very similar to the embodiment of FIGS. 7 and 8. The difference being that the portion of the operation device placed in the main portion M comprises a different transmission in which the worm drive is replaced by a transmission comprising regular gear wheels 137 engaging a part of a gear wheel 132 integrated in the third and fourth members 101c, 101d, respectively. In difference to the embodiment shown in FIGS. 7 and 8, the flexible shafts 135 transferring mechanical rotating force from the motors MO1, MO2 of the remote unit 140, does not enter the main portion M through the second member 101b, but rather centrally in the main portion M, in a direction perpendicular to the planar extension of the medical device 10. Entering the main portion M directly through the enclosure EN of the main portion M, enables all of the members 101a, 101b, 101c, 101d be flexible in the second direction D2 substantially perpendicular to the first direction D1 for adapting the medical device 10 to the curvature of the stomach S of the patient and/or for facilitating insertion of the medical device 10 into the body of the patient. I.e. all of the first, second, third and fourth members 101a, 101b, 101c, 101d are hinged at a first, second and third pivotal points 26a′,26b′,26c′,26a″,26b″,26c″,26a″′,26b″′,26c″′, 26a″″,26b″″,26c″″ for enabling pivotal movement.

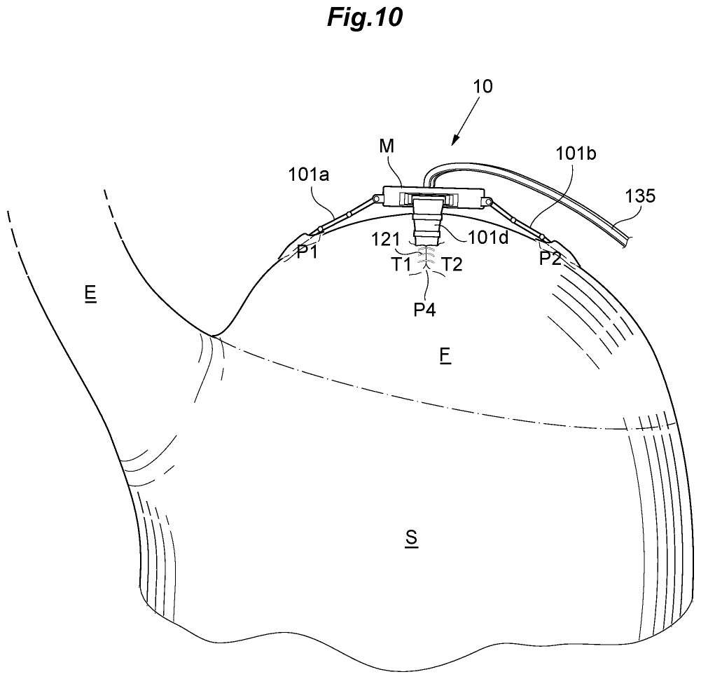

FIG. 10 shows the embodiment of FIG. 9, when the first, second, third and fourth members 101a, 101b, 101c, 101d have been invaginated by the tissue of the wall of the fundus F part of the stomach S. Tissue T1, T2 of the stomach wall has been folded over the distal portions of the members 101a, 101b, 101c, 101d and sutured 121 or stapled 121 together. This means that first tissue T1 connects to second tissue T2 over the members 101a, 101b, 101c, 101d making it possible for the connected tissue T1, T2 of the stomach wall to grow together creating a stable and reliable long term fixation even if the sutures 121 or staplers 121 are resorbed or rejected by the body. When implanted, fixated and in function, the operation device of the medical device 10 operates the fourth member 101d to displace in relation to the first and second members 101a, 101b. In a first state, the operation device operates the third member such that the third member 101c is displaced in a direction towards the second member 101b, which increases the distance between the third member 101c and the first member 101a, which thereby increases the distance between a third portion P3 of the stomach wall, to which the third member 101c is fixated, and a first portion P1 of the stomach wall, to which the first member 101a is fixated. The increasing distance between the third and first portions P3, P1 stretches the tissue of the stomach wall between the third and first portions P3, P1 which activates the stretch receptors in the stomach wall creating a feeling of satiety in the patient. In a second state, the operation device operates the third member such that the third member 101c is displaced in a direction towards the first member 101a, which increases the distance between the third member 101c and the second member 101b, which thereby increases the distance between a third portion P3 of the stomach wall, to which the third member 101c is fixated, and a second portion P2 of the stomach wall, to which the second member 101b is fixated. The increasing distance between the third and second portions P3, P2 stretches the tissue of the stomach wall between the third and second portions P3, P2 which activates the stretch receptors in the stomach wall creating a feeling of satiety in the patient.

Having the medical device 10 alter the portions of the stomach wall which are stretched ensures that the efficiency of the medical device 10 is maintained over time, as the stretch receptors of a specific portion of the stomach S may become less receptive to stretching if the same portion of the stomach wall is stretched all of the time.

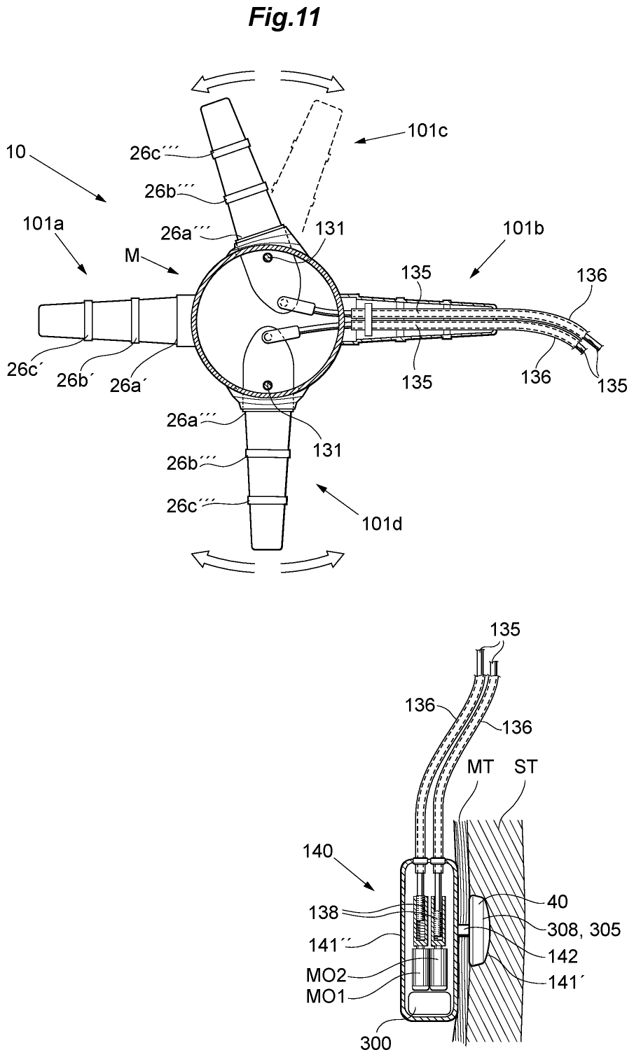

FIG. 11 shows an embodiment of the medical device 11 very similar to the embodiment of FIGS. 7 and 8. The difference being that the flexible shafts 135 are Bowden cables 135,136 configured to transfer linear force from the remote unit 140 to the main portion M of the medical device 10. The motors MO1, MO2 in the remote unit 140 are connected to nut-and-screw transmission elements 138 transferring the rotating force created by the motors MO1, MO2 to a linear force to be transferred by means of the Bowden cables 135,136 to the portion of the operation device placed in the main portion M of the medical device 10. The flexible shaft 135 of the Bowden cables 135,136 enters the main portion M through the second member 101b, forming a receiving portion, and are pivotally connected to the third and fourth members 101c, 101d such that the linear movement created by the Bowden cable makes the third and fourth members 101c, 101d pivot around the pivotal point 131 for operating the third and fourth members 101c, 101d for stretching the stomach wall of the patient.

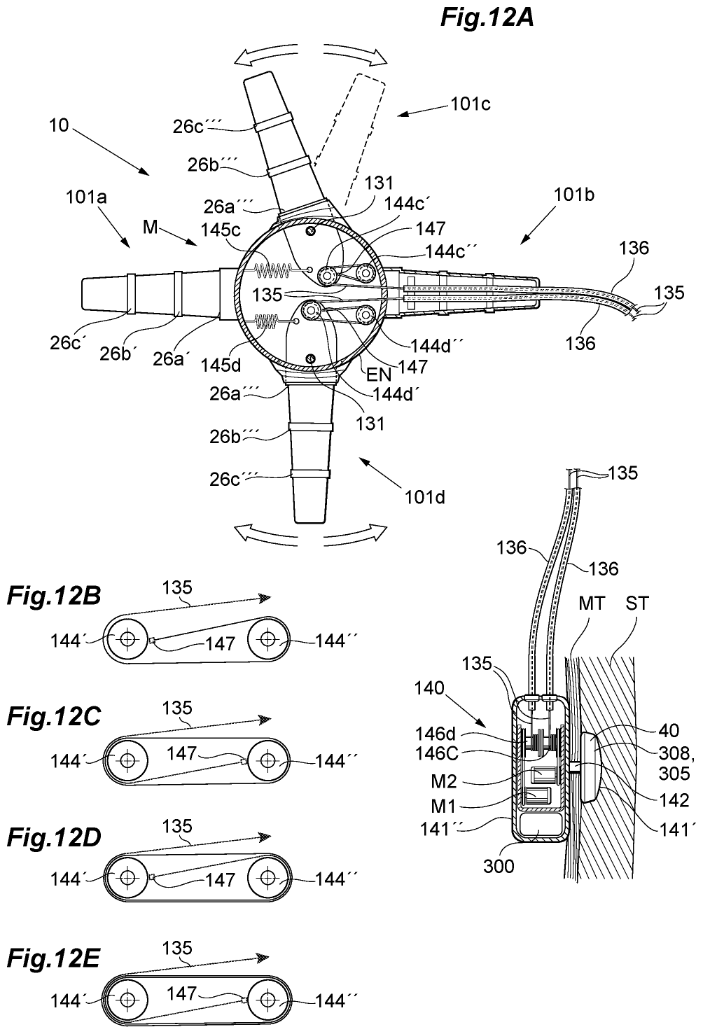

FIG. 12a shows an embodiment of the medical device 10 very similar to the embodiment of FIG. 11. The difference being that the portion of the operation device placed in the main portion M of FIGS. 12a-12e is equipped with block and tackle functionality providing transmission. The cable 135 that transfer force from the remote unit 140 to the main portion M is flexible and could be a metal wire made from a plurality of strands or could be made from a flexible polymer material. The transmission provided by the block and tackle functionality enables the third and fourth member 101c, 101d to operate with a mechanical force having higher force and lower speed than the mechanical force generated at the remote unit 140. In the embodiment shown in FIG. 12a, the remote unit 140 comprises a first motor MO1 which is connected via a belt drive with a first spool or drum 146d on which the cable 135 is rolled on and off. In the same way, a second motor MO2 is connected by means of a belt drive to a second spool or drum 146c on which a second cable 135 is rolled on and off. The cable 135 from the first spool or drum 146d runs from the remote unit 140, in a protective cover 136, through the second member 101b into the main portion M. In the main portion M, the cable 135 is threaded on a first pulley 144d′ which is rotatably fixated to the fourth member 101d. The cable 135 is then threaded onto a second pulley 144d″, rotatably fixated to the enclosure EN enclosing the main portion M, and is finally fixated to the fourth member 101d. When the cable 135 is pulled a first distance with a first force at the remote unit 140, the fourth member 101d is actuated by the first pulley 144d′ moving towards the second pulley 144d″ half that distance but with twice the force. The cable 135 from the second spool or drum 146c runs from the remote unit 140, in a protective cover 136, through the second member 101b into the main portion M. In the main portion M, the cable 135 is threaded on a first pulley 144c′ which is rotatably fixated to the third member 101c. The cable 135 is then threaded onto a second pulley 144c″, rotatably fixated to the enclosure EN enclosing the main portion M, and is finally fixated to the third member 101c. When the cable 135 is pulled a first distance with a first force at the remote unit 140, the third member 101c is actuated by the first pulley 144c′ moving towards the second pulley 144c″ half that distance but with twice the force. The third and fourth members 101c, 101d are moved in the opposite direction by means of springs 145c,145d connected to the third and fourth members 101c, 101d respectively. The springs 145c,145d also creates pre-tension in the cable 135 which eliminates slack end ensures that the cables remain threaded onto the pulleys 144c′,144c″,144d′,144d″ and the spools/drums 146c,146d at all time.

FIG. 12b shows an isolated view of the block and tackle functionality for operating the fourth member 101d, shown in FIG. 12a. The cable 135 runs from the first pulley 144′ to the second pulley 144″ and is finally fixated at a fixation point 147 at the first pulley 144′. Pulling the cable 135 a first distance with a first force results in the first pulley 144′ moving towards the second pulley 144″ half that distance but with twice the force. This embodiment is a known as a gun tackle and uses the principle rove to advantage, which means that the first pulley 144′ being rotatably fixated to the fourth member 101d moves in the same direction as the force acting on the cable 135, but with an increased strength.

FIG. 12c shows an isolated view of an alternative block and tackle functionality, in which the first pulley 144′ is a double pulley. The cable 135 runs from the first pulley 144′ to the second pulley 144″, back to the first pulley 144′ and is finally fixated at a fixation point 147 at the second pulley 144″. Pulling the cable 135 a first distance with a first force results in the first pulley 144′ moving towards the second pulley 144″ ⅓ of that distance but with three times the force. This embodiment is a known as a luff or watch tackle and uses the principle rove to advantage, which means that the first pulley 144′ rotatably fixated to the fourth member 101d moves in the same direction as the force acting on the cable 135, but with an increased strength.

FIG. 12d shows an isolated view of an alternative block and tackle functionality, in which both the first and second pulleys 144′, 144″ are double pulleys. The cable 135 runs from the first pulley 144′ to the second pulley 144″, back to the first pulley 144′, back to the second pulley 144″ and is finally fixated at a fixation point 147 at the first pulley 144′. Pulling the cable 135 a first distance with a first force results in the first pulley 144′ moving towards the second pulley 144″ ¼ of that distance but with four times the force. This embodiment is a known as a double tackle and uses the principle rove to advantage, which means that the first pulley 144′ rotatably fixated to the fourth member 101d moves in the same direction as the force acting on the cable 135, but with an increased strength.

FIG. 12e shows an isolated view of an alternative block and tackle functionality, in which the first pulley 144′ is a triple pulley and the second pulley 144″ is a double pulley. The cable 135 runs from the first pulley 144′ to the second pulley 144″, back to the first pulley 144′, back to the second pulley 144″, again back to the first pulley 144′ and is finally fixated at a fixation point 147 at the second pulley 144′. Pulling the cable 135 a first distance with a first force results in the first pulley 144′ moving towards the second pulley 144″ ⅕ of that distance but with five times the force. This embodiment is a known as a gyn tackle and uses the principle rove to advantage, which means that the first pulley 144′ rotatably fixated to the fourth member 101d moves in the same direction as the force acting on the cable 135, but with an increased strength.

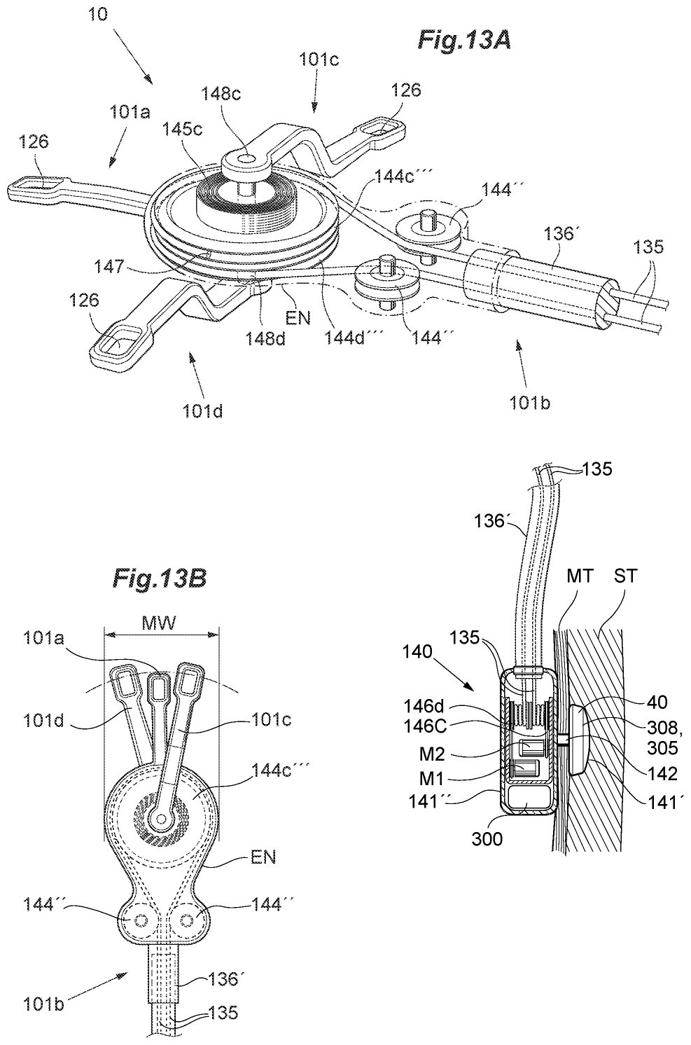

FIG. 13a shows an embodiment of the medical device 10 very similar to the embodiment of FIG. 12a. The difference being that the portion of the operation device placed in the main portion M of FIGS. 12a-12b is equipped with larger pulleys 144c″′,144d″′. The larger pulleys 144c″′,144d″′ are connected to shafts 148c,148d which exits the enclosure EN in the center of the top and bottom portion of the enclosure EN. The shafts 148c,148d extends in a direction perpendicular to the extension of the plane of the stomach wall along which the medical device 10 is placed. The shafts 148c,148d are fixated to the third and fourth members 101c, 101d respectively and as such operates the third and fourth members 101c, 101d to stretch the stomach wall. Just as in the embodiment of FIG. 12a, the remote unit 140 pulls and releases a cable 135 which is flexible and could be a metal wire made from a plurality of strands or could be made from a flexible polymer material.

A cable 135 is pulled and released by a drum 146c operated by a second motor MO2. The cable 135 travels from the remote unit 140 through a protective sheet 136′ configured to house two cables 135. The cable 135 enters the main portion M of the medical device 10 through the second member 101b and is guided by an idler pulley 144″. The cable 135 then partially wraps around the upper pulley 144c″′ of the two larger pulleys and is fixated to the upper pulley 144c″′ at a fixation point 147 such that the upper pulley 144c″′ rotates when the cable 135 is pulled. The pulling of the cable 135 connected to the upper pulley 144c″′ operates the third member 101c such that the third member 101c is displaced in a direction towards the second member 101b, which increases the distance between the third member 101c and the first member 101a, which stretches the tissue of the stomach wall between the third member 101c and the first member 101a which activates the stretch receptors in the stomach wall creating a feeling of satiety in the patient. The operation device of the embodiment of FIG. 13a further comprises a torsion spring 145c fixated to the upper pulley 144c″′ and indirectly fixated to the enclosure EN, such that pulling of the cable 135 tightens the torsion spring 145c creating the force that is used for operating the third member 101c in an opposite direction displacing the third member 101c in a direction towards the first member 101a, which increases the distance between the third member 101c and the second member 101b, which stretches the tissue of the stomach wall between the third member 101c and the second member 101b which activates the stretch receptors in the stomach wall creating a feeling of satiety in the patient. The torsion spring 145c also creates pre-tension in the cable 135 which eliminates slack end ensures that the cable 135 remain threaded onto the pulleys 144c″,144c″′ and the drum 146c at all time.

Another cable 135 is pulled and released by a drum 146d operated by a first motor MO1. The cable 135 travels from the remote unit 140 through a protective sheet 136′ configured to house two cables 135. The cable 135 enters the main portion M of the medical device 10 through the second member 101b and is guided by an idler pulley 144″. The cable 135 then partially wraps around the lower pulley 144d″′ of the two larger pulleys and is fixated to the lower pulley 144d″′ at a fixation point (hidden) such that the lower pulley 144d″′ rotates when the cable 135 is pulled. The pulling of the cable 135 connected to the lower pulley 144d″′ operates the fourth member 101d such that the fourth member 101d is displaced in a direction towards the second member 101b, which increases the distance between the fourth member 101d and the first member 101a, which stretches the tissue of the stomach wall between the fourth member 101d and the first member 101a which activates the stretch receptors in the stomach wall creating a feeling of satiety in the patient. The operation device of the embodiment of FIG. 13a further comprises a torsion spring (hidden) which in the same way as the torsion spring 145c is fixated to the lower pulley 144d″′ and indirectly fixated to the enclosure EN, such that pulling of the cable 135 tightens the lower torsion spring creating the force that is used for operating the fourth member 101d in an opposite direction displacing the fourth member 101d in a direction towards the second member 101b, which increases the distance between the fourth member 101d and the second member 101b, which stretches the tissue of the stomach wall between the fourth member 101d and the second member 101b which activates the stretch receptors in the stomach wall creating a feeling of satiety in the patient. The lower torsion spring also creates pre-tension in the cable 135 which eliminates slack end ensures that the cable 135 remain threaded onto the pulleys 144d″,144d″′ and the drum 146d at all time.

In the embodiment of FIGS. 13a and 13b, the larger pulley 144c″′,144d″′ creates transmission as a larger pulley means that the cable 135 needs to travel a longer distance to generate the same radial movement of the pulley 144c″′,144d′ and thereby of the shaft 148c, which in turn means that such longer distance can be traveled using a lower force. In the embodiment shown in FIGS. 13a and 13b, the central portion of the drums 146c,146d has a diameter which is smaller than the diameter of the pulleys 144c″′,144d″′. Specifically, in the embodiment shown in FIGS. 13a and 13b, the drums 146c,146d have a diameter of 1 mm and the pulleys 144c″′,144d″′ have a diameter of 10 mm. This means that 10 revolutions of the drums 146c,146d generates 1 revolution of the pulleys 144c″′,144d′ generating a 10/1 transmission reducing the required force to 1/10, at the same time as the speed needs to be increased 10 times. In alternative embodiments, the transmission may be more than 2/1, more than 4/1, more than 6/1 or more than 20/1.

FIG. 13b shows the embodiment described with reference to FIG. 13a, when placed in a state for insertion into the body of the patient. The cables 135 have been completely released such that the torsion springs pulls the third and fourth members 101c, 101d towards the first member 101a, such that the third and fourth members are placed within the width MW of the main portion M of the medical device 10 for facilitating the insertion of the medical device 10 into the body of the patient in either open surgery, laparoscopic surgery or natural orifice transluminal endoscopic surgery (NOTES) using for example a gastroscope.

In the embodiment shown in FIGS. 13a and 13b, the members 101a, 101b, 101c and 101d all comprises through holes 126 for the fixation of the members 101a, 101b, 101c, 101d to the stomach wall using sutures or staplers. However, this particular feature of the medical device may be assisted or replaced by any of the fixation methods disclosed herein.

In the embodiments shown in FIGS. 12a-13b, the third and fourth members 101c, 101d are actuated by means of a pulling force from the Bowden cables in a first direction and by means of forces released by springs in the second direction. However, in alternative embodiments it is equally conceivable that the Bowden cables are of a push pull type, such that they are able to transfer force both in the push direction and in the pull direction. Such embodiments eliminate the need for springs or other elastic elements in the operation of the third and fourth members 101c, 101d.

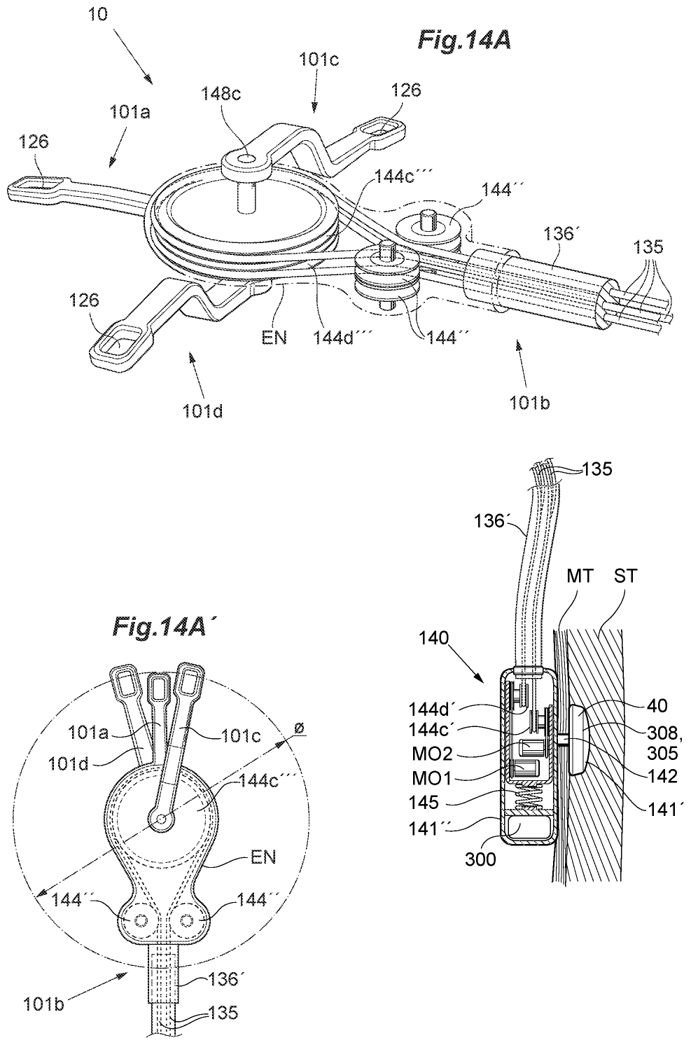

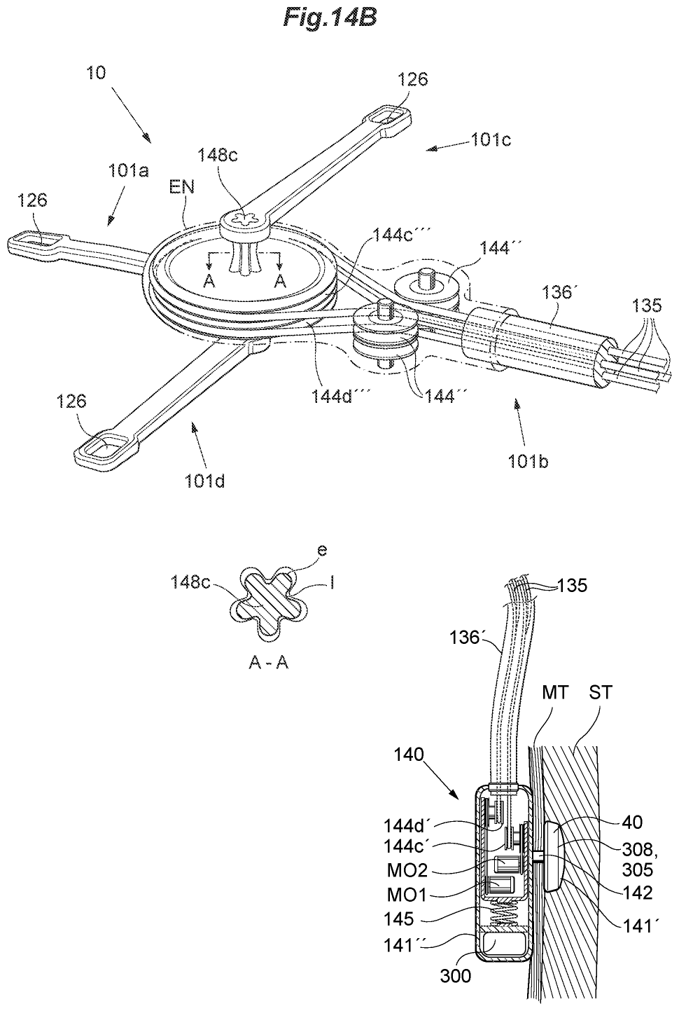

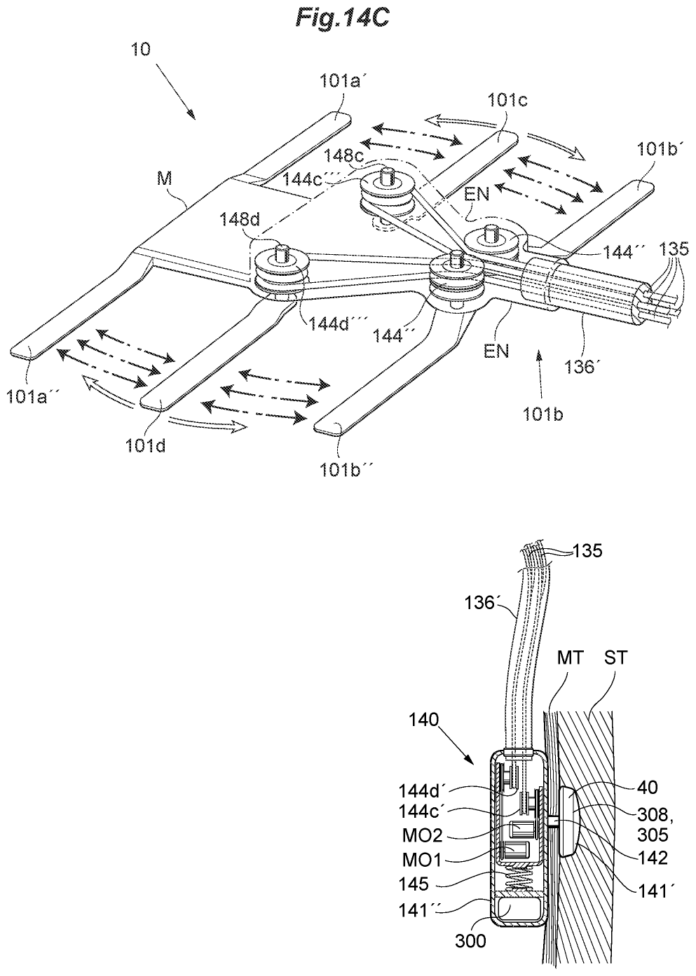

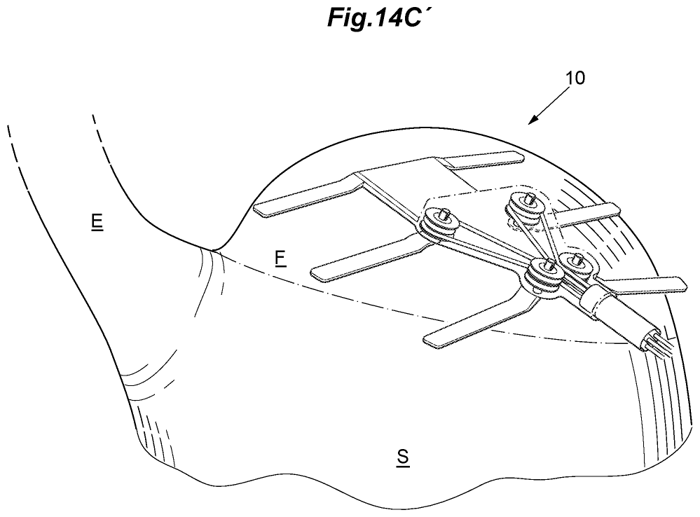

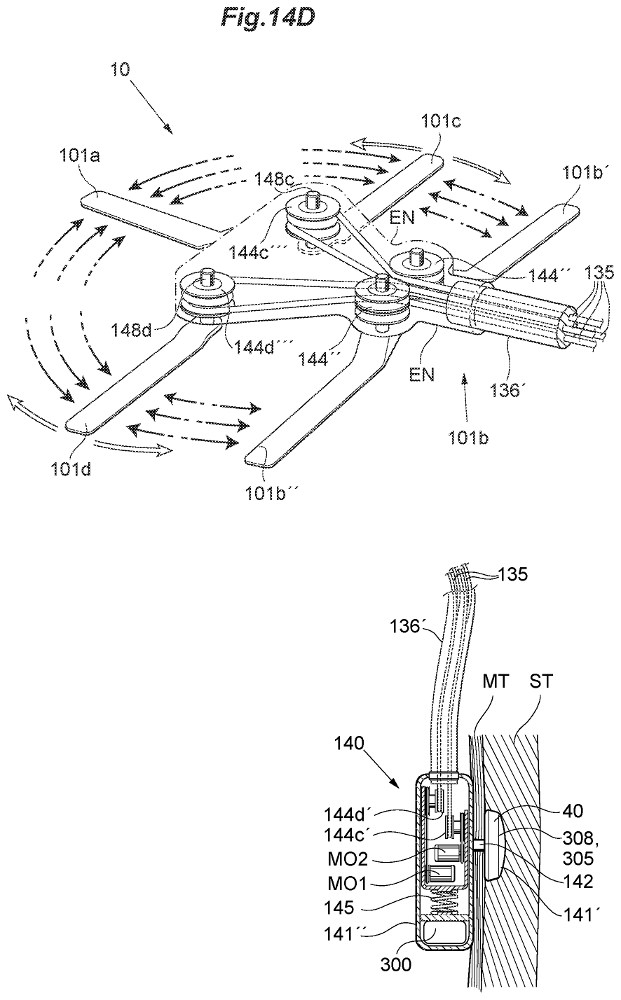

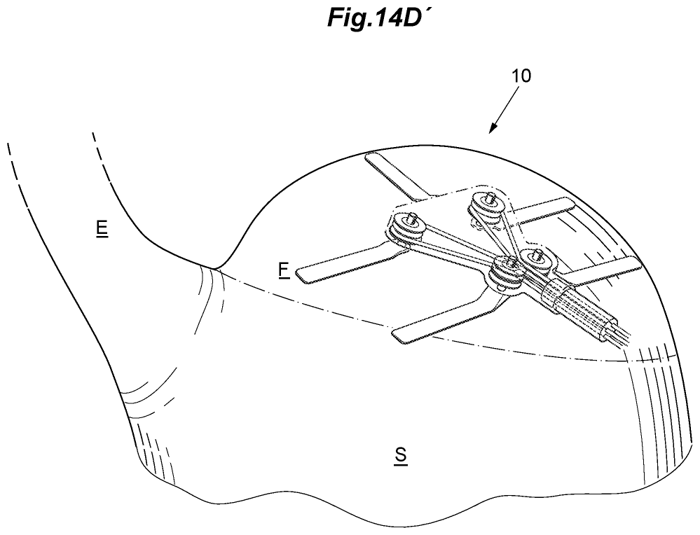

FIGS. 14a and 14a′ shows an embodiment of the medical device similar to the embodiment shown in FIGS. 13a and 13b. However, in the embodiment of FIGS. 14a and 14a′, the cables 135 are in the form of loops, running around the larger pulleys 144c″,144d″ and around smaller pulleys 144c′,144d′ in the remote unit 140. This means that the third and fourth members 101c, 101d can be displaced in both directions by pulling action by the smaller pulleys 144c′,144d′ in the remote unit 140. As such, no spring is needed to displace the larger pulleys 144c″′,144d″ in any direction. In the remote unit 140 the smaller pulleys 144c′,144d′ are operated by a first and second motor MO1, MO2 by means of belt drive. The motors MO1, MO2 are suspended and pulled back by a spring 145 which creates pre-tension in the cables 135 which eliminates slack end ensures that the cables 135 remain threaded onto the pulleys 144d′,144d′,144c″′,144d″′ all time. The cables 135 is pulled in two directions by the smaller pulleys 144c′,144d′ operated by the first and second motors MO1, MO2. The cables 135 travel from the remote unit 140 through a protective sheet 136′ configured to house four cables 135. The cables 135 enter the main portion M of the medical device 10 through the second member 101b and is guided by double idler pulleys 144″. The cables 135 are then looped around the larger upper and lower pulleys 144c″′,144d″′ such that the upper and lower pulleys 144c″′,144d″′ rotates when the cables 135 are pulled.

In the embodiment of FIGS. 14a and 14a′, the larger pulleys 144c″′,144d″′ creates transmission as a larger pulley means that the cable 135 needs to travel a longer distance to generate the same radial movement of the pulley. This in turn means that a longer distance can be traveled using a lower force. In the embodiment shown in FIGS. 14a and 14a′, the central portion of the pulleys 144c′,144d′ has a diameter which is smaller than the diameter of the larger pulleys 144c″′,144d″′. Specifically, in the embodiment shown in FIGS. 14a and 14a′, the smaller pulleys 144c′,144d′ have a diameter of 3 mm and the larger pulleys 144c″′,144d″′ have a diameter of 10 mm. This means that 10 revolutions of the smaller pulleys 144c′,144d′ generates 10/3 revolution of the larger pulleys 144c″′,144d″′ generating a 10/3 transmission reducing the required force to 0,3, at the same time as the speed needs to be increased 10/3 times. In alternative embodiments, the transmission may be more than 2/1, more than 4/1, or more than 6/1.

FIG. 14b shows an embodiment being very similar to that of FIGS. 14a,14a′. The difference with FIG. 14b is that the displaceable members 101c, 101d are straight and extends perpendicularly in relation to a length axis of the shaft 148c. The members 101c, 101d being straight separates the movement of the members 101c, 101d further from the enclosure EN of the main portion M which may be beneficial when the main portion and the displaceable members 101c, 101d are covered by fibrotic tissue. FIG. 14b also differs in that the shaft 148c has a waist and a cross-section having elevated e and lowered 1 portions, such that the circumference is made longer. The longer circumference and the waist on the shaft 148c facilitate rotation of the shaft 148c when the shaft 148c has been overgrown with fibrotic tissue. This will mean that the fibrotic tissue will be longer and as such need to extend less for enabling the same rotation of the shaft 148c. In the embodiment shown in FIG. 14b, the circumference ϕ of the shaft 148c at the cross-section A-A having a cross-sectional area A have a relationship such that ϕ>3π*sgrt(A/π).

Having straight members 101c, 101d however also means that they will pivot in two different planes, perpendicular to the length axis of the shafts 148c. The pivoting will also be in a different plane from the plane in which the first and second members 101a, 101b extend (that plane also being perpendicular to the length axis of the shaft 148c).

FIG. 14c shows an embodiment of the medical device, similar to the embodiment shown with reference to FIGS. 13a-14b. In the embodiment shown in FIG. 14c, the first member extends in two opposing portions 101a′, 101a″, each portion being fixedly and immovably fixated to the main portion M and extending perpendicularly from the main portion M. The second member also extends in two opposing portions 101b′, 101b″, each portion being fixedly and immovably fixated to the main portion M and extending perpendicularly from the main portion M. Placed centrally between the portions 101a′, 101a″, 101b′, 101b″, of the first and second members is a pivotable third member 101c and a pivotable fourth member 101d. The third and fourth members 101c, 101d are each fixated to a pulley or capstan 144c″′,144d″′ having shafts 148c,148d rotatably fixated to the main portion M. The flexible wire 135 runs from the remote unit (further disclosed with reference to FIG. 14a), via the idler pulleys 144″ to the pulleys or capstans 144c″′,144d″′ fixated to the third and fourth members 101c, 101d respectively. The flexible wires 135 loops around the capstans 144c″′,144d″′ creating the required friction between the flexible wires 135 and the capstans 144c″′,144d″′. The embodiment shown in FIG. 14c creates a relatively large area of stomach tissue which can be stretched, which leads to a reduction of the risk that the receptiveness of the stretch receptors of that specific area is disappears. If the receptiveness of the stretch receptors in the tissue wall placed between the outermost portions of the members 101a′, 101c, 101b′, 101a″, 101d, 101b″ is decreased, the stretching will increase on the portions of tissue wall placed closer to the main portion M and consequently, the receptors placed in those portions of the stomach wall will increasingly be the receptors creating the sensation of satiety. As such, the embodiment shown in FIG. 14c have a high chance of functioning in the long term. In the embodiment shown in FIG. 14c, the pivoting members 101c, 101d extends parallelly to the members 101a′, 101b′, 101a″, 101b″ being fixedly fixated to the main portion M. Also, in the embodiment shown in FIG. 14c, the distance thatthe members 101a′, 101c, 101b′, 101a″, 101d, 101b″ extends perpendicularly from the main portion M exceeds the distance between the first members 101a′, 101a″ and the third and fourth members respectively, and between the second members 101b′, 101b″ and the third and fourth members respectively, as such, a relatively long stretching distance is created between the main portion M and the outermost portions of the members 101a′, 101c, 101b′, 101a″, 101d, 101b″.

FIG. 14c′ shows the embodiment of the medical deice 10 described with reference to FIG. 14c, when positioned and fixated to the stomach S of the patient. As previously discussed, the stretch receptors are mostly located in the fundus region F of the stomach S and as such, stretching the fundus region has the most effect on the satiety sensation felt by the patient. As the medical device 10 of the embodiment of FIG. 14c becomes quite large, it may be so that the entire device cannot be placed in the fundus F region, rather a portion of the device may have to be placed in the corpus region of the stomach S in which stretching may be less efficient. Just as in other embodiments, the members may be fixated to the stomach wall by means of invagination, or by means of sutures, staplers or growth promoting structures. It may also be so that the entire medical device 10 is invaginated by the stomach wall, such as shown in FIG. 16′.

FIG. 14d shows an embodiment of the medical device similar to the embodiment shown with reference to FIGS. 14c and 14c′. However, in the embodiment shown in FIG. 14d, the first members 101b′, 101b″ have been replaced by a single first member 101a fixedly fixated to the main portion M.

The single first member 101a is fixated to the main portion M such that it extends perpendicularly to the third and fourth members 101c, 101d. As such, the tissue of the stomach wall placed between the third and fourth member 101c, 101d is stretched basically in a direction perpendicular to the extension of the third and fourth members 101c, 101d, whereas the tissue placed between the third and fourth members 101c, 101d and the single first member 101a is stretched in a direction at an angle substantially 450 from the extension of the single first member 101a. This means that the stretching of the tissue wall placed between the second members 101b′, 101b″ and the third and fourth members 101c, 101d will be more reliable for a longer time period, whereas the angled stretching between the third and fourth members and the single first member 101a risks becoming less effective as the stretch receptors becomes less receptive.

FIG. 14d′ shows the medical device 10 when positioned in the fundus region of the stomach.

The medical device according to the embodiment shown in FIG. 14d is somewhat smaller than the embodiment of the medical device shown in FIG. 14c. As such, a larger portion of the medical device 10 is positioned in the fundus region, which means that the stretching stomach wall with the use of the device according to FIG. 14d can be performed to a larger extent in the fundus region in which houses more of the stretch receptors. Just as in other embodiments, the members 101a, 101b′, 101b″, 101c, 101d may be fixated to the stomach wall by means of invagination, or by means of sutures, staplers or growth promoting structures. It may also be so that the entire medical device 10 is invaginated by the stomach wall, such as shown in FIG. 16′.

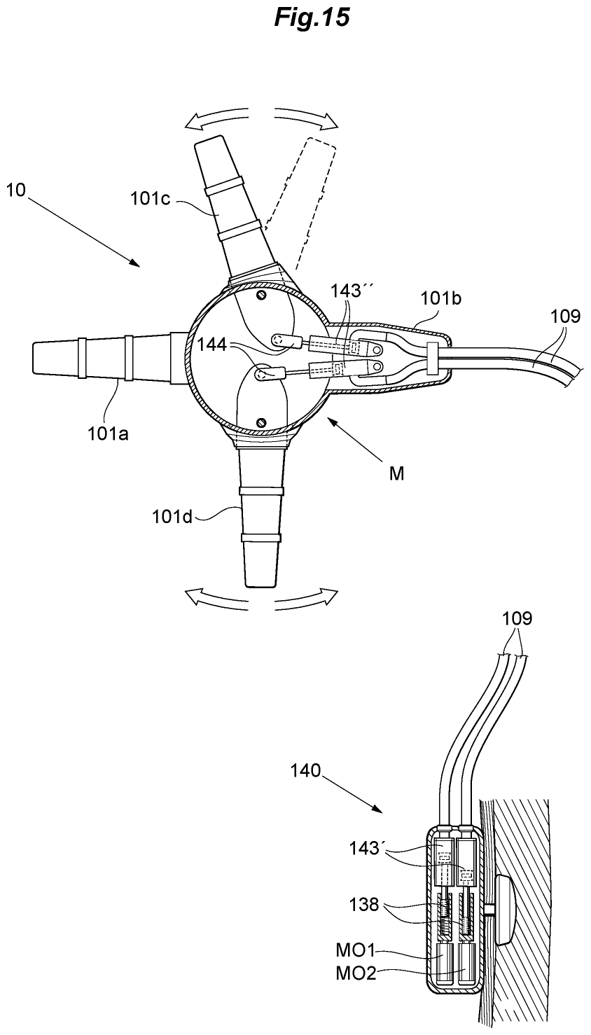

FIG. 15 shows an embodiment of the medical device 10 very similar to the embodiment of FIGS. 11 and 12a. The difference being that in the embodiment of FIG. 13, the operation device is a hydraulic operation device, such that the force transferred from the remote unit 140 to the main portion M of the medical device 10 is hydraulic force. The flexible shaft of the embodiments of FIGS. 7-12e are replaced by conduits 109 for conducting hydraulic fluid for transferring force from the remote unit 140 to the portion of the operation device placed in the main portion M of the medical device 10 hydraulically.

In the embodiment of FIG. 15, in the remote unit 140, a first and second motor MO1, MO2, are each connected to a nut-and-screw transmission element 138 transferring the rotating force created by the motors MO1, MO2 to a linear force which in turn affects two first, remote, hydraulic cylinders 143′. The two first remote hydraulic cylinders 143′ are in fluid connection with the conduits 109 such that hydraulic fluid and thereby force is conducted from the first cylinders 143′ through the conduits 109 to the second cylinders 143″ positioned in the second member 101b. The second cylinders 143″ are in mechanical connection with the third and fourth members 101c, 101d, by means of a hydraulic piston placed in the second hydraulic cylinders 143″ and are pivotally connected to the third and fourth members 101c, 101d forming a transmission of force, such that operation of the second cylinders 143″ operates the third and fourth members 101c, 101d for stretching the stomach wall.

FIG. 16 shows the medical device 10 when positioned against the stomach wall of the patient.

The medical device 10 could be the medical device according to any one of the embodiments of FIGS. 4A 6, having an integrated operation device 100, controller 300 and energy storage unit 40. The controller 300 is connected to a sensor 106 for sensing food intake of the patient. In the embodiment shown in FIG. 16, the sensor is a acoustic sensor in the form of a microphone connected to the esophagus and configured to pick up sounds in the esophagus connected to the patient swallowing. The acoustic signals generated by the sensor 106 are filtered and processed by the controller 300 and is used as input to the controller for controlling the operation device 100. The controller 300 could as a result of the sensor input control the medical device on the basis of the food intake of the patient. The controller 300 can be programmed to control the operation device on the basis of the amount of food consumed by the patient, the frequency of food intake, the time at which food is consumed or the shape or viscosity of the food intake. I.e., the patient drinking generates a different acoustic profile from the patient eating solid food, which can be used as an input to control the medical device to stretch the stomach wall to create a feeling of satiety. As an example, a patient's diet may be based on the patient only eating at certain fixed times during the day. In such an example, the medical device 10 can be programmed and controlled to immediately stretch the stomach wall to create a feeling of satiety if the patient consumes food at any other time during the day. In the embodiment shown in FIG. 16, the sensor 106 is attached to the esophagus E. In alternative embodiments, the sensor 106 may be placed encircling the esophagus E, which is a suitable embodiment when the sensor is a sensor measuring expansion of the esophagus E as a result of the patient swallowing, for example using a strain sensor which may be a piezo electric or piezo resistive sensor. The sensor could in the alternative be another type of motility sensor, such as an accelerometer measuring the motility of the esophagus E as the patient swallows. In yet another alternative, an optical sensor may be used for sensing the opacity alteration over the esophagus as food passes. The controller may comprise at least one unit having a sleep mode and an active mode, and the unit consumes less energy in the sleep mode than in the active mode. The unit is configured to switch from the sleep mode to the active mode on the basis of at least one signal from the acoustic sensor. The unit could for example be a DSP (Digital Signal Processor), another type of processor or a wake-up circuit of the controller, which in turn activates the functions of the controller. The unit may be configured to switch from the sleep mode to the active mode on the basis of a signal from the acoustic sensor related to the patient swallowing a number of times and/or on the basis of a signal from the acoustic sensor related to the patient swallowing a number of times during a time period. The number of times the patient swallows and the time could be counted/measured and compared with a pre-set or moving threshold value. The controller could further comprise at least one filtering unit configured to filter acoustic signals related to at least one of: speech, the swallowing of saliva and chewing. The filter could be a digital filter implemented as hardware or software in the controller and could have the filter characteristics of a high, low or bandpass filter.

In yet alternative embodiments, the sensor may be fixated at a location which is not immediately in connection with the esophagus, such as at a portion of the patient comprising bone, such as the sternum or a rib of the patient, or on the outside of the skin of the patient, by means of for example an adhesive patch or a band encircling the torso of the patient. Fixating the sensor to the anterior side of the sternum or a rib of the patient may be advantageous as the sensor is more accessible, which means that maintenance or replacement of the sensor could be performed with minimally invasive surgery. The sensor fixated to the sternum or a rib of the patient could be an acoustic sensor, as sound propagates well though bone, or could be a piezo electric or piezo resistive sensor or an accelerometer. Fixated to the sternum or the at least one rib of the patient could be performed on the anterior side thereof or on the posterior side thereof. An accelerometer could be an accelerometer configured to measure acceleration in one plane, two planes or three planes, such that movement such as vibrations of e.g. the sternum can be measured in one, two or three axis, which can be used to separate the swallowing of solid foods from the swallowing of fluids, and separate the speech from swallowing and movement from swallowing. For this purpose, a controller of the medical implant, or a controller external to the body of the patient could use input from the sensor, such as the accelerometer and run through a filtering unit in which of the internal or external controller, such that motion patterns linked to speech and movement can be filtered from motion patterns being linked to swallowing, and the swallowing of fluids can be distinguished from the swallowing of solids.

In yet alternative embodiments, the sensor may be fixated external to the body of the patient, such as at a location which on the sternum or ribcage on the surface of the skin. The sensor may be connected to the skin by means of an adhesive patch or by means of a band encircling the torso of the patient. The sensor could be an acoustic sensor, as sound propagates well though bone, or could be a piezo electric or piezo resistive sensor or an accelerometer. The accelerometer could be an accelerometer configured to measure acceleration in one plane, two planes or three planes, such that movement such as vibrations of e.g. the sternum can be measured in one, two or three axis, which can be used to separate the swallowing of solid foods from the swallowing of fluids, and separate the speech from swallowing and movement from swallowing. For this purpose, a controller for example placed in a smart watch or smart phone could use input from the sensor, such as the accelerometer and run through a filtering unit in the controller, such that motion patterns linked to speech and movement can be filtered from motion patterns being linked to swallowing, and the swallowing of fluids can be distinguished from the swallowing of solids.

The sensor may be configured to sense at least one parameter related to a functional status of the medical device, such as at least one parameter related to the operation of the operation device.

In yet alternative embodiments, the sensor may be configured to sense the acidity in the stomach. In such an embodiment, the sensor is preferably placed on the inside of the stomach wall, inside of the acidic environment of the stomach. As the acidity in the stomach increases when the patient eats, the acidity may be used as an indicator of the food intake of the patient.

The sensor, or a further sensor could be configured to sense a temperature of the medical device 10 or a local or systemic temperature of the patient. The temperature of the patient could be used to assess the health of the patient, such the health problems resulting from the implanted medical device can be avoided. The temperature of the medical device can be an indication of the function of the medical device. 10. If the strain in the medical device 10 increases, heat generated by the operation device 100 may increase, which could be a sign that something in the medical device 10 is not functioning properly. This is also true for the power consumption of the medical device 10, which may be measured by a sensor and/or monitored by the controller 300.

The controller 300 could be configured to receive further input from sensors configured to sense for example blood oxygenation, blood pressure, or a parameter related to an ischemia marker such as lactate.

FIG. 16′ shows the medical device 10 of the embodiment described with reference to FIG. 16.

In FIG. 16′, the medical device 10 has been completely invaginated by the tissue of the stomach wall, such that the growing-together of tissue covering the medical device 10 fixates the medical device 10 in the long term and protects the medical device 10 from the environment in the abdomen.

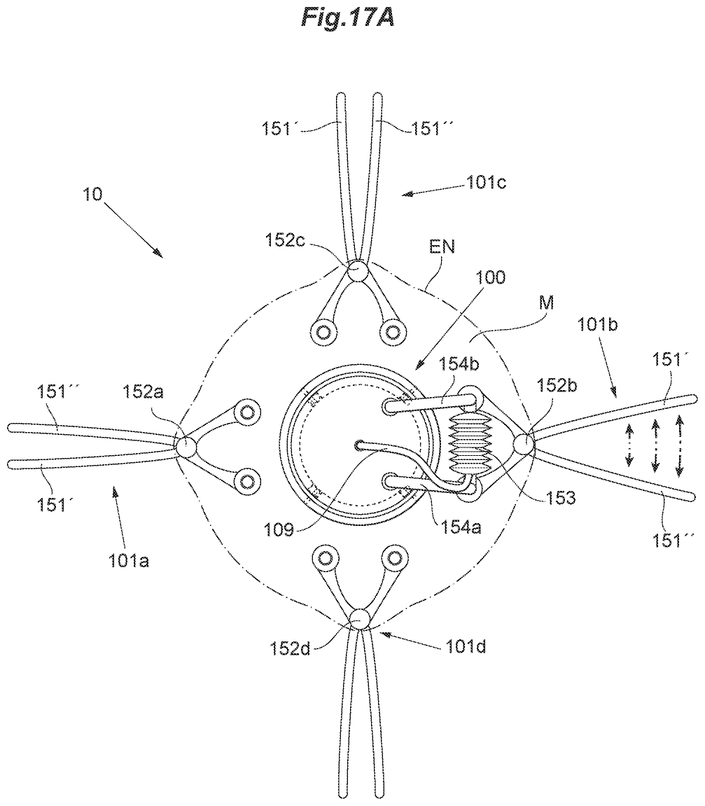

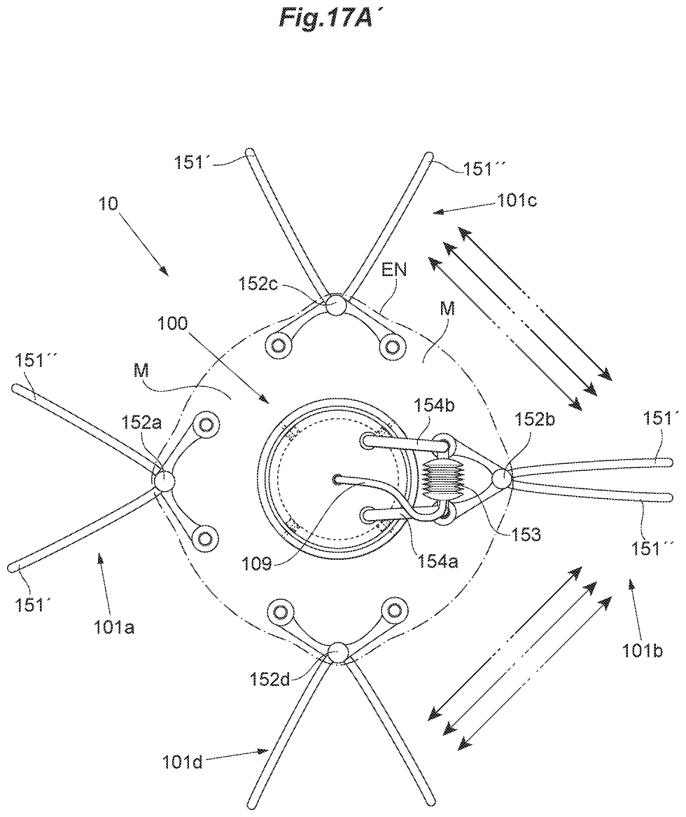

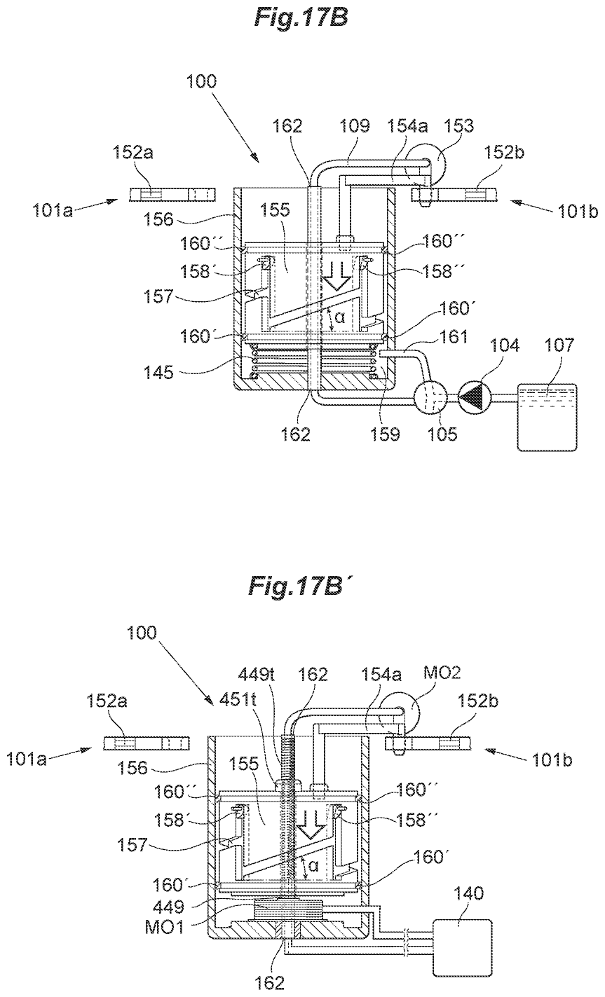

FIG. 17a shows an embodiment of the medical device 10 in a plane top view. The medical device 10 of the embodiment shown in FIG. 17a is a hydraulic medical device comprising four scissor-like members 101a, 101b, 101c, 101d each being fixated at a point of pivot 152a,152b,152c,152d at a main portion M and each comprising two elongated stomach engagers 151′,151″ pivotably fixated to the main portion M at the points of pivot 152a,152b,152c,152d, and thus sharing points of pivot 152a,152b,152c,152d. The stomach engagers 151′,151″ may be fixated to the outer surface of the stomach wall, just as described in other embodiments herein, by means of invagination, suturing, stapling or by means of in-growth of fibrotic tissue. The four scissor-like members 101a, 101b, 101c, 101d are in the embodiment shown in FIG. 17a operated by a single hydraulic operation device 100 that alternates between the scissor-like members 101a, 101b, 101c, 101d for operating each of the scissor-like members 101a, 101b, 101c, 101d individually. The hydraulic operation device 100 comprises an actuator in the form of an expandible bellows 153 placed between a first and second pivotable lever 154a,154b and connected to a conduit 109 for hydraulic fluid. In the state shown in FIG. 17a, the bellows 153 is connected to the second scissor-like member 101b at the handle-like operating ends of each of the two elongated stomach engagers 151′,151″. In the state shown in FIG. 17a, the second scissor-like member 101b is operated by the transportation of hydraulic fluid to and from the bellows 153 by the hydraulic fluid conduit 109. The movement of hydraulic fluid to the bellows 153 causes the bellows 153 to expand and thereby urging the two elongated stomach engagers 151′,151″ to move apart from each other in the end of the two elongated stomach engagers 151′,151″ engaging the stomach wall. The moving apart of the two elongated stomach engagers 151′,151″ stretches the stomach wall placed between the two elongated stomach engagers 151′,151″. The operation device 100 is enclosed by an enclosure EN which encircles and enclosed the operation device just outside of the points of pivot 152a,152b,152c,152d, such that the points of pivot 152a,152b,152c,152d are sealed from bodily fluids and the ingrowth of fibrotic tissue.

FIG. 17a′ shows an embodiment of the medical device similar to that of the embodiment of FIG. 17a. The difference being that in the embodiment of FIG. 17a′, the idle state of the scissor-like members 101a, 101b, 101c, 101d is one in which the elongated stomach engagers 151′,151″ are displaced away from each other, i.e., scissor-like members 101a, 101b, 101c, 101d are “open” in the idle state. In the state shown in FIG. 17a′, the actuator in the form of the bellows 153 have been operated such that hydraulic fluid has been moved from the bellows 153 causing the bellows to contract and “close” the second scissor-like member 101b displacing the first stomach engager 151′ of the second member 101b towards the second stomach engager 151″ of the second member 101b, for stretching the stomach wall portion between the second and third members 101b, 101c and the stomach wall portion between the second and fourth members 101b, 101d.

FIG. 17b shows the medical device 10 of the embodiment of FIG. 17b in a cross-sectional side view in which the operation device 100 is shown in further detail. In the embodiment of FIG. 17b, the operation device is configured to displaced the actuator (bellows 153) by a combination of linear and rotational movement, for displacing the actuator for a first state in which the actuator is configured for operating the second member 101b, to a second state in which the actuator is configured for operating the third member 101c. Starting from below in the figure, the operation device comprises a hydraulic cylinder 156, in which a piston 155 is positioned. The piston 155 is aligned and sealed against the inner walls of the cylinder 156 by means of lower seals 160′ and upper seals 160″. The piston 155 is moved up in the cylinder 156 by a hydraulic force created by an implantable pump 104 pumping hydraulic fluid from a fluid conduit 161 into the chamber 159 of the cylinder 156 placed below the piston 155. The movement of the piston 155 in the upwards direction is against the counter-acting force of the spring 145 which is fixated to the bottom of the piston 155 and to the bottom of the inside of the cylinder 156, for pulling the cylinder downwards. The piston 155 comprises a groove 157 having a slanted portion with an angle α, in which steering members 158′,158″ are configured to run. The steering members 158′,158″ are rotatably fixated to the inner walls of the cylinder 156 and are configured to control the rotation of the piston 155 in the cylinder 156 and thereby move the actuator in form of a bellows 153. The implantable pump 104 pumps fluid from a fluid reservoir 107. The fluid is guided by an electrically controllable valve 105 to either the inlet 161 and further into the chamber 159 for operating the piston 155, or to the bellows 153. The fluid conduit 109 operating the bellows 153 runs from the electrically controllable valve 105 through a channel 162 traveling though the cylinder 156 and the piston 155 and exiting the top portion of the piston 155 in the center thereof. The implantable pump could for example be one of the implantable pumps described with reference to FIGS. 54a-58.

In the state shown in FIGS. 17a and 17b, the spring 145 has pulled the piston 155 to its lowest position in the cylinder 156. This means that the pivotable levers fixated to the top portion of the piston 155 are in a position in which the pivotable levers engages the operating ends of each of the two elongated stomach engagers 151′,151″, such that the actuator in form of a bellows 153 can operate the operating ends of the two elongated stomach engagers 151′,151″ for stretching the stomach wall.

FIG. 17b′ shows an embodiment of the medical device similar to that of the embodiment of FIG. 17b. The difference being that in the embodiment of FIG. 17b′, the operation device 100 is a mechanical operation device and the bellows has been replaced by an electrical motor MO2 (which may be replaced by a solenoid), in the shown state, operating the second member 101b for stretching the stomach wall. The chamber and spring of the embodiment of FIG. 17b has been replaced by an electrical motor MO1 and a force output 449 in the form of a hollow shaft being threaded 449t. The threaded force output 449t engages a correspondingly threaded portion 451t of the piston having internal threads. The threaded force output 449t transforms the rotational movement crated by the electrical motor MO1 to a linear force acting on the piston 155 for moving the piston 155 up and down in the cylinder 156 for performing the same movement pattern as described in relation to the embodiment of FIG. 17b. The motor MO2, in the same way comprises a transmission for transforming rotational movement crated by the electrical motor MO2 to a linear force acting on the second member 101b. The mechanical actuator in the form of the electrical motor MO2 may comprise at least one flexible member covering the motor MO2. The flexible member comprising elevated and lowered portions configured to enable the motor MO2 to be operable when fibrotic tissue is covering the elevated and lowered portions. The first and second electrical motors MO1, MO2 are connected to electrical leads, which connects the motors to a controller and an energy storage unit comprises in a remote unit 140, details of which is further described with reference to FIGS. 7-15 and 33-53b.

In alternative embodiments, at least one of the electrical motors MO1, MO2 may be positioned at a remote location in relation to the main portion M, and the medical device may then comprise a force transferring member transferring rotational or linear force to the main portion for displacing and operating the actuator. The transferring of force from a remote unit to a main portion is for example further described with reference to FIGS. 7-14.

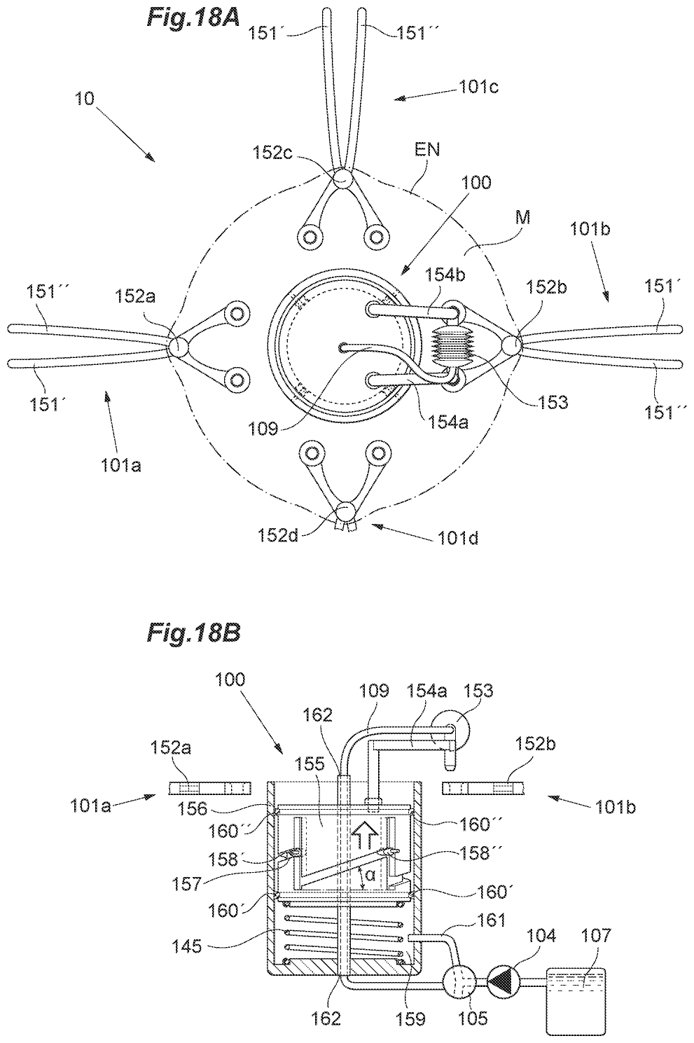

FIGS. 18a, 18b shows the medical device 10 according to the embodiment shown in FIGS. 17a,17b in a state in which the hydraulic pump 104 has pumped hydraulic fluid into the chamber 159 such that the piston 155 has moved half-way in the cylinder 156. In this position the steering members 158′,158″ rotates by means of a string action such that the protruding ends of the steering members 158′,158″ enters the slanted portions of the groove 157 that runs radially at an angle α along the outer surface of the piston 155. The piston 155 being moved half-way up in the cylinder 156 causes the pivotable levers 154a,154b to move up-wards together with the piston, causing the pivotable levers 154a,154b to be lifted from their engagement in the operating ends of the two elongated stomach engagers 151′,151″, such that the pivotable levers 154a,154b are disconnected from the operating ends of the two elongated stomach engagers 151′,151″.

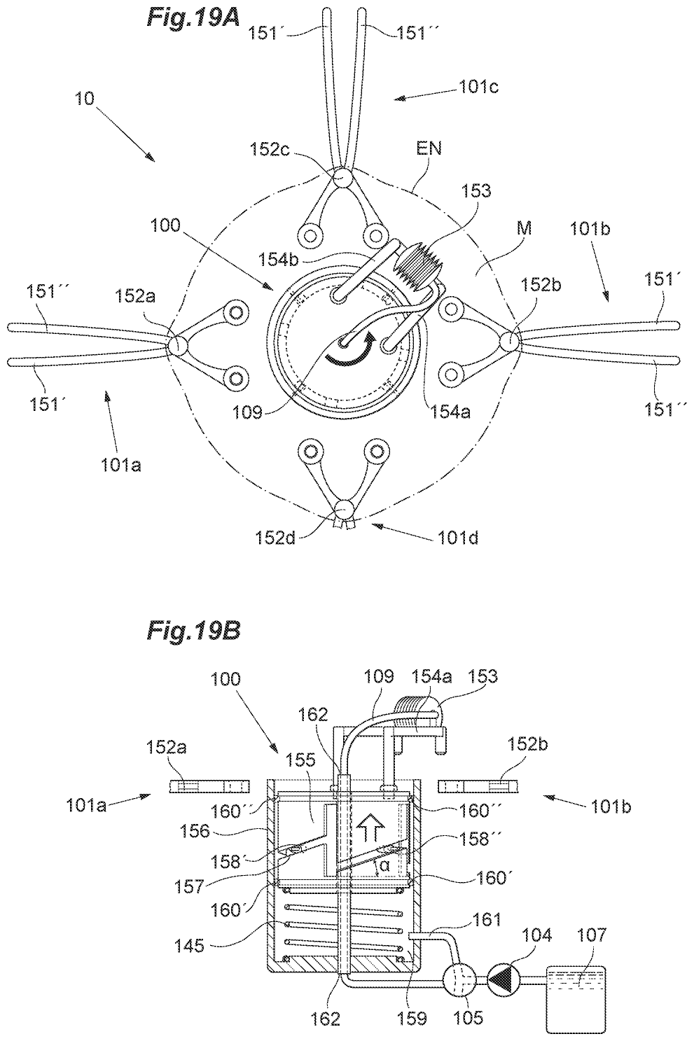

FIG. 19a, 19b shows the medical device 10 according to the embodiment shown in FIGS. 17a-18b in a state in which the hydraulic pump 104 has pumped hydraulic fluid into the chamber 159 such that the piston 155 has moved 3% to the top in the cylinder 156. The angle of the groove 157 causes the piston 155 to rotate in the cylinder 154 which causes the pivotable levers 154a,154b and the operable bellows 153 to rotate to a new position. In FIGS. 19a,19b, the pivotable levers 154a,154b and the operable bellows 153 is in a position between the position for operating the second scissor-like members 101b and the position for operating the third scissor-like members 101c.

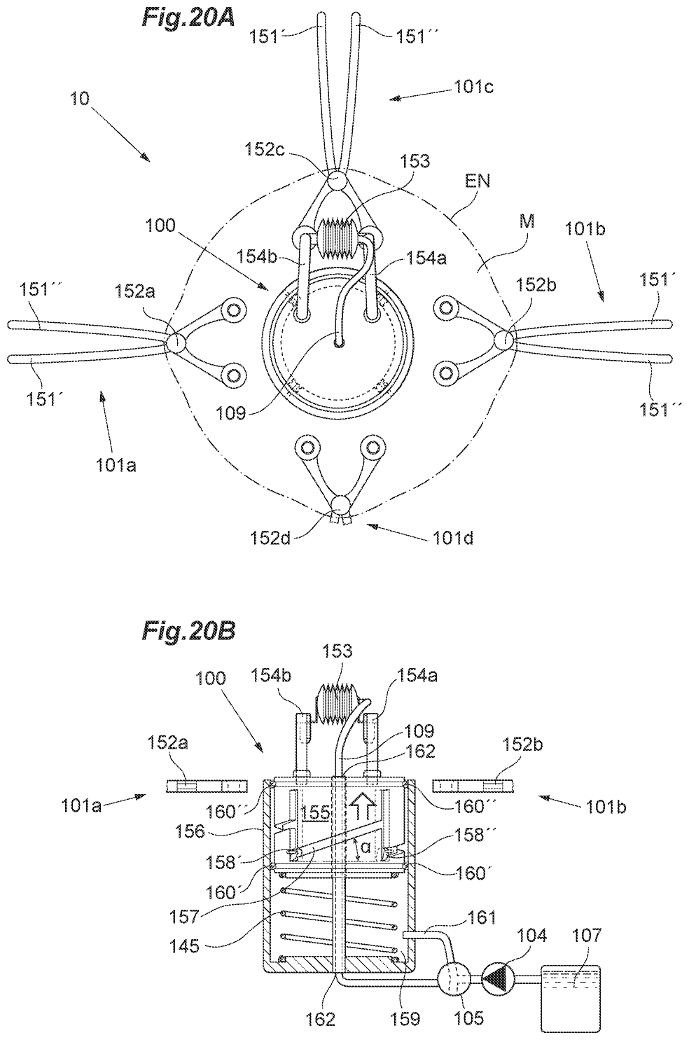

FIG. 20a, 20b shows the medical device 10 according to the embodiment shown in FIGS. 17a-19b in a state in which the hydraulic pump 104 has pumped hydraulic fluid into the chamber 159 such that the piston 155 has moved all the way to the top in the cylinder 156. The angle of the groove 157 has now caused the piston 155 to rotate turn in the cylinder 154 which has caused the pivotable levers 154a,154b and the operable bellows 153 to rotate to a new position aligned with the third scissor-like members 101c. The steering members 158′,158″ are now positioned in the bottom of the groove 157 and have moved along the angled radial portion of the groove 157. In the bottom position the steering members 158′,158″ are now in a position in which they will travel straight up along the axial portion of the groove 157 when the piston 155 travels back down. When the piston 155 is lowered from the position, the pivotable levers 154a,154b engages the third scissor-like members 101c and places the bellows 153 in an operational mode in which the pivotable levers 154a,154b and the bellows 153 can operate the third scissor-like members 101c to stretch the stomach wall of the patient such that satiety is created.

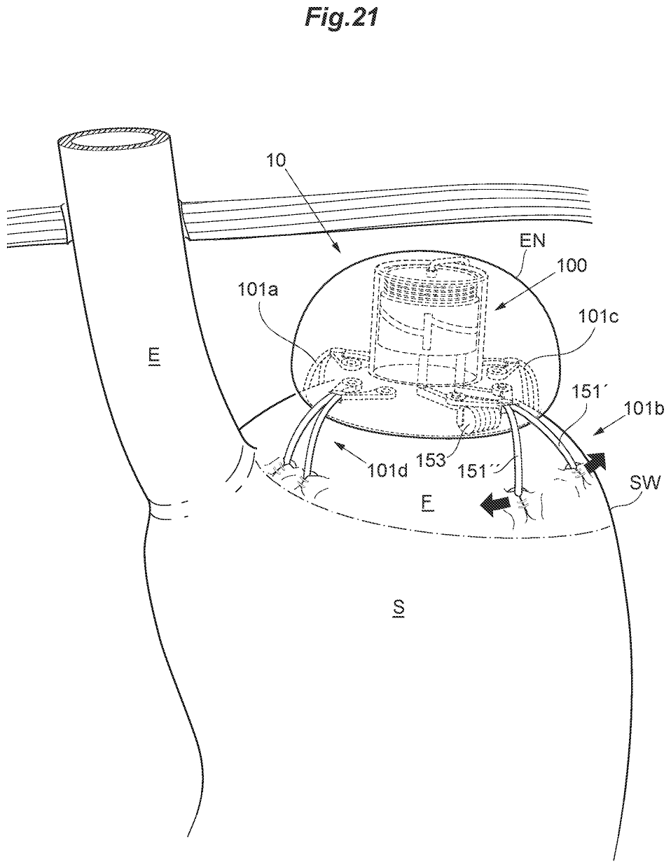

FIG. 21 shows the medical device according to the embodiment shown in FIGS. 17a-20b when implanted in the patient's body and fixated to the outside of the stomach wall SW of the fundus F of the patient. In the state shown in FIG. 21, the operation device 100 of the medical device 10 operate the second scissor-like member 101b for moving the two elongated stomach engagers 151′,151″ away from each other for stretching the stomach wall placed between the two elongated stomach engagers 151′,151″.

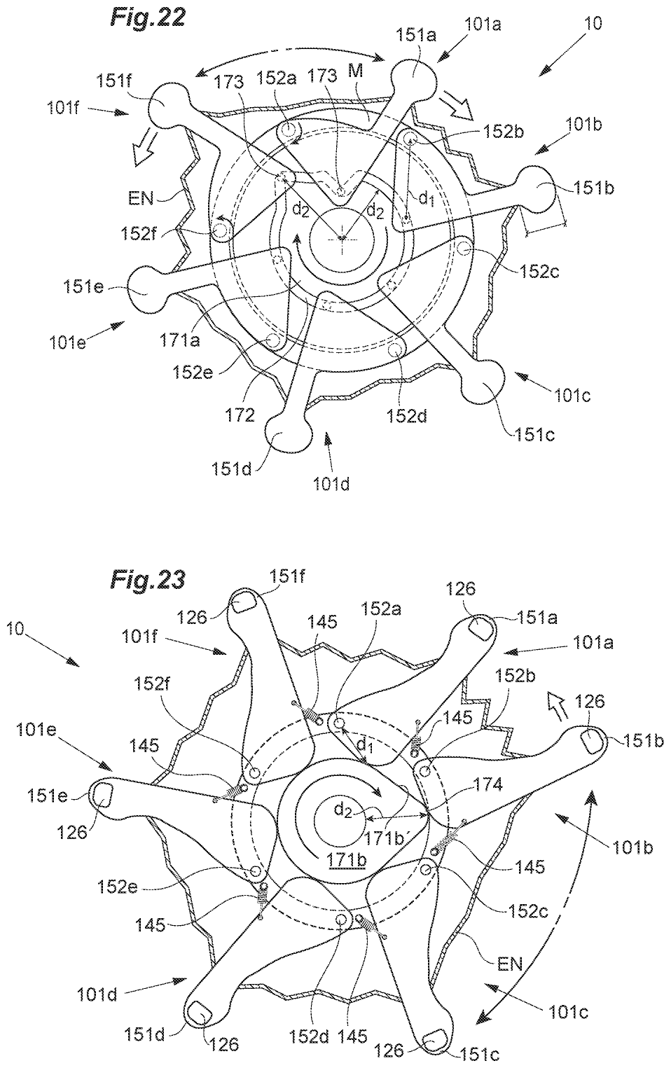

FIG. 22 shows an embodiment of the medical device 10 in a plane top view. The medical device 10 of the embodiment shown in FIG. 22 is an implantable mechanical medical device comprising six members 101a, 101b, 101c, 101d, 101e, 101f each being fixated to a main portion M at points of pivot 152a,152b,152c,152d,152e,152f and each comprising a stomach engager 151a,151b,151c,151d,151e,151f. In the embodiment of FIG. 22, the stomach engagers 151a,151b,151c,151d,151e,151f are configured to be fixated to a first, second, third, fourth, fifth and sixth portion of the stomach wall by the stomach engagers being invaginated by the stomach wall using stomach-to-stomach sutures or staplers. However, just as described in other embodiments herein, the fixation can be performed by means of invagination, suturing, stapling or by means of in-growth of fibrotic tissue. The six members 101a, 101b, 101c, 101d, 101e, 101f are operated by an operation device comprising an eccentric rotatable engagement member 171a. In the embodiment of FIG. 22, the eccentric rotatable engagement member 171a comprises a recess in the form of a groove 172 encircling the center of rotation of the eccentric rotatable engagement member 171a and being configured to engage cylindrical protrusions 173 fixated to each of the six members 101a, 101b, 101c, 101d, 101e, 101f, at a distance d1 from the points of pivot 152a,152b,152c,152d,152e,152f. The cylindrical protrusions 173 are configured to slide in, and be guided by, the groove 172, causing the distance d2, between the center of rotation of the eccentric rotatable engagement member 171a and the cylindrical protrusions 173, to vary, which in turn causes the members 101a, 101b, 101c, 101d, 101e, 101f to pivot in relation to the main portion M. The groove 172 thus controls the movement of the members 101a, 101b, 101c, 101d, 101e, 101f both in a first direction, increasing the distance d2, and in the opposite direction, decreasing the distance d2. As such, the groove is configured to control the movement of the members 101a, 101b, 101c, 101d, 101e, 101f at all times. Both the groove and the cylindrical protrusions 173 are made from a stiff durable material. Preferably the groove 172 is made in a metallic material and the cylindrical protrusions 173 are made from a self-lubricating polymer material, such as PTFE, PFA, FEP or UHMWPE. In the alternative, the groove 172 may be formed in a self-lubricating polymer material and the cylindrical protrusions 173 may be made from a metallic material. In alternative embodiments, the groove 172 and/or the cylindrical protrusions 173 may be formed form a ceramic material, such as zirconium oxide. As the eccentric rotatable engagement member 172 comprising the groove rotates, cylindrical protrusions 173 fixated to the members 101a, 101b, 101c, 101d, 101e, 101f causes sequential displacement of the members 101a, 101b, 101c, 101d, 101e, 101f in relation to the main portion M for sequentially stretching the stomach wall portions between pairs of the members. This means, that as the groove 172 rotates: the first and second members 101a, 101b are simultaneously displaced, such that the stomach engager 151a of the first member 101a moves towards the stomach engager 151f of the sixth member 101f and the stomach engager 151b of the second member 101b moves towards the stomach engager 151c of the third member 101c. When the groove 172 has completed another ⅙ revolution the second and third members 101b, 101c are simultaneously displaced, such that the stomach engager 151b of the second member 101b moves towards the stomach engager 151a of the first member 101a and the stomach engager 151c of the third member 101c moves towards the stomach engager 151d of the fourth member 101d. When the groove 172 has completed another ⅙ revolution the third and fourth members 101c, 101d are simultaneously displaced, such that the stomach engager 151c of the third member 101c moves towards the stomach engager 151b of the second member 101b and the stomach engager 151d of the fourth member 101d moves towards the stomach engager 151e of the fifth member 101e. When the groove 172 has completed another ⅙ revolution the fourth and fifth members 101d, 101e are simultaneously displaced, such that the stomach engager 151d of the fourth member 101d moves towards the stomach engager 151c of the third member 101c and the stomach engager 151e of the fifth member 101e moves towards the stomach engager 151f of the sixth member 101f. When the groove 172 has completed another ⅙ revolution the fifth and sixth members 101e, 101f are simultaneously displaced, such that the stomach engager 151e of the fifth member 101e moves towards the stomach engager 151d of the fourth member 101d and the stomach engager 151f of the sixth member 101f moves towards the stomach engager 151a of the first member 101a. When the groove 172 has completed another ⅙ revolution the sixth and first members 101f, 101a are simultaneously displaced, such that the stomach engager 151f of the sixth member 101f moves towards the stomach engager 151e of the fifth member 101e and the stomach engager 151a of the first member 101a moves towards the stomach engager 151b of the second member 101b.

In the embodiment of FIG. 22, the main portion M and the points of pivot 152a,152b,152c,152d,152e,152f are enclosed by an enclosure EN which is a flexible enclosure EN comprising elevated and lowered areas, such that the members can be operated to stretch the stomach wall even as fibrotic tissue grows on the flexible enclosure EN. The flexible enclosure EN comprising elevated and lowered areas could comprise a pleated portion, such as a bellows. The distance between the “peaks” of two elevated areas may be in the interval 2 mm-6 mm, or in the interval 2 mm-5 mm and the elevation difference between the lowest point of a lowered area to a peak may be in the interval 1 mm 6 mm, or in the interval 1 mm-5 mm.

In the embodiment shown in FIG. 22, the operation device comprises an electrical motor for propelling the eccentric rotatable engagement member 171a such that it rotates. The electrical motor is connected to the eccentric rotatable engagement member 171a via a transmission in the form of a gear system configured to reduce the speed and increase the force of the movement generated by the electrical motor.

FIG. 23 shows an embodiment of the medical device similar to the embodiment shown in FIG. 22. The difference is that the embodiment of FIG. 23 comprises five members 101a, 101b, 101c, 101d, 101e pivotally connected to the main portion M and an eccentric rotatable engagement member 171b comprising a radial engagement surface 171b′ configured to radially engage the first, second, third, fourth and fifth members 101a, 101b, 101c, 101d, 101e such that the eccentric rotation of the engagement member 171b causes the members 101a, 101b, 101c, 101d, 101e to pivot in relation to the main portion M.

In the embodiment shown in FIG. 23, each member 101a, 101b, 101c, 101d, 101e is also connected to the main portion M by means of an elastic element 145 in the form of a spring 145. The members 101a, 101b, 101c, 101d, 101e are configured to be operated in the first direction by the eccentric rotation of the engagement member 171b and in an opposite direction by the elastic elements 145. Just as in FIG. 22, the eccentric rotatable engagement member 171b is propelled by an electrical motor connected to the eccentric rotatable engagement member 171a via a transmission in the form of a gear system configured to reduce the speed and increase the force of the movement generated by the electrical motor.

In the embodiment shown in FIG. 23, the eccentric rotatable engagement member 171b comprises a single eccentric portion 174 having a longer distance d2 from the center of rotation of the eccentric rotatable engagement member 171b. As such, only one member 101a, 101b, 101c, 101d, 101e at a time is pivoted such that the pivoted member causes in increase in the distance between the pivoted member and the member ahead of the pivoted member, in a clockwise orientation. It is however conceivable, in alternative embodiments that the eccentric rotatable engagement member 171b also comprises a portion adjacent to the eccentric portion in which the distance to the center of rotation of the eccentric rotatable engagement member 171b is shorter, such that a member adjacent to the member pivoted by the eccentric portion 174 is pivoted in the opposite direction by the force of the elastic member 145. Having one member pivoting in the first direction and another member pivoting in a second direction makes the distance increases larger and thus increases the stretching effect.

In the embodiment of FIG. 23, each of the stomach engagers 151a,151b,151c,151d,151e, comprises a fixation portion comprising a through-hole 126 for receiving sutures or staplers for fixating each of the stomach engagers 151a,151b,151c,151d,151e to a respective portion of the stomach wall.

In the embodiments shown in FIGS. 22 and 23, the electrical motor and gear box are placed directly in the main portion M, however, in alternative embodiments it is equally conceivable that part of the operation device is placed in the main portion M of the medical device, and part of the operation device is placed remotely, such that the medical device is remotely powered with mechanical force. The remote operation device could for example be a remote operation device according to any of the embodiments of FIGS. 7-15.

Just as in the other embodiments disclosed herein, the embodiments of FIGS. 22 and 23 comprises an energy storage unit and controller for directly or indirectly energizing and controlling the medical device. The energy storage unit and controller can either be placed locally, in the main portion M of the medical device, or remotely in a remote unit (such as the remote unit in any of the embodiments of FIGS. 7-15). Features of the remote unit and the controller are further described with reference to FIGS. 33-65f.

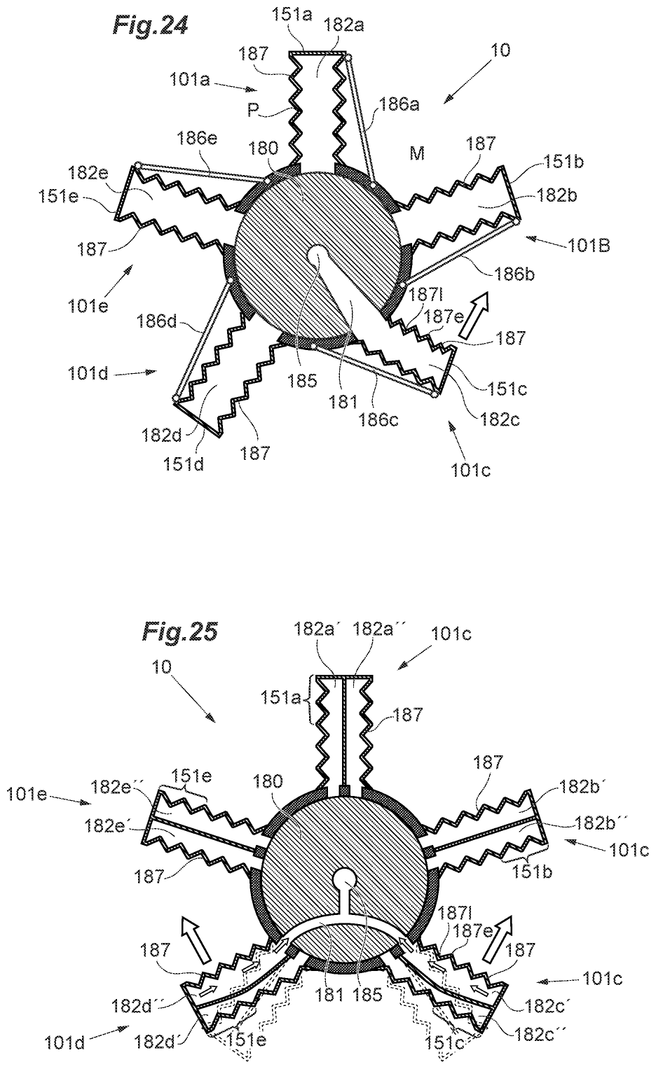

FIG. 24 shows an embodiment of the medical device 10 in a plane top view. The medical device 10 of the embodiment shown in FIG. 24 is an implantable hydraulic medical device comprising five members 101a-101e each being fixated to a main portion M and each being operable to compress and expand in relation to the main portion M, by hydraulic fluid being moved to and from fluid chambers 182a-182e enclosed by flexible wall portions 187 of each of the members 101a-101e. Each member comprises a stomach engager portion 151a-151e and each is hingedly fixated to a pivoting element 186a-186e, which in turn is pivotally fixated to the main portion M. The compression and expansion of the five members 101a-101e causes the pivoting elements 186a-186e to pivot when hydraulic fluid is moved to or from the fluid chambers 182a-182e which causes the stomach engager portions 151a-151e to displace in relation to each other, causing a stretching of the stomach wall between the stomach engager portions 151a-151e.

In the embodiment shown in FIG. 24, the flexible walls 187 comprises pleated portions P having elevated and lowered portions 187e,1871 enabling the members 101a-101e to bend also when fibrotic tissue is covering the elevated and lowered portions 187e,1871. The distance between the “peaks” of two elevated portions may be in the interval 2 mm-6 mm, or in the interval 2 mm-5 mm and the elevation difference between the lowest point of a lowered portion to a peak may be in the interval 1 mm-6 mm, or in the interval 1 mm-5 mm.

In the embodiment of FIG. 24, the stomach engagers portions 151a-151e are configured to be fixated to a first, second, third, fourth, fifth and sixth portion of the stomach wall by the stomach engager portions being invaginated by the stomach wall using stomach-to-stomach sutures or staplers. However, just as described in other embodiments herein, the fixation can be performed by means of invagination, suturing, stapling or by means of in-growth of fibrotic tissue. The five members 101a, 101b, 101c, 101d, 101e are operated by means of a rotating valve 180 configured to move a fluid conduit 181 such that each of the members can be placed in fluid connection with a fluid conduit 181.

In the state shown in FIG. 24, the rotating valve 180 is positioned such that the fluid conduit 181 is connected to the fluid chamber 182c of the third member 101c. The conduit 181 is further connected to a central conduit, which in turn is connected to a hydraulic pump (not shown), such as the hydraulic pumps described with reference to FIGS. 54a-58. In the state shown in FIG. 24, the hydraulic pump has sucked hydraulic fluid from the chamber 182c of the third member 101c, causing the third member to compress and thereby bend by the actuation of the pivoting element 186c, in a direction towards the stomach engager portion 151b of the second member 101b, and away from the stomach engager portion 151d of the fourth member 101d. The stomach engager portion 151c of the third member 101c moving away from the stomach engager portion 151d of the fourth member 101d creates a stretching of the stomach wall between the third and fourth members 101c, 101d.

The rotation of the rotating valve 180 causes a new chamber to be connected to the fluid conduit 181 and thereby being operated for bending. In this way the medical device sequentially stretches five different portions of the stomach wall such that satiety can be created without inducing fatigue or loss of sensitivity to stretching at a specific portion of stomach wall.

FIG. 25 shows an embodiment of the medical device 10 in a plane top view. The medical device 10 of the embodiment shown in FIG. 25 is an implantable hydraulic medical device comprising five members 101a, 101b, 101c, 101d, 101e each being fixated to a main portion M and operable to bend in relation to the main portion M. Each member comprises a stomach engager portion 151a,151b,151c,151d,151e. In the embodiment of FIG. 24, the stomach engagers portions 151a,151b,151c,151d,151e are configured to be fixated to a first, second, third, fourth, fifth and sixth portion of the stomach wall by the stomach engager portions being invaginated by the stomach wall using stomach-to-stomach sutures or staplers. However, just as described in other embodiments herein, the fixation can be performed by means of invagination, suturing, stapling or by means of in-growth of fibrotic tissue. The five members 101a, 101b, 101c, 101d, 101e are operated by means of a rotating valve 180 configured to move a fluid conduit 181 such that the members can be placed in fluid connection with the fluid conduit 181 in pairs. Each member 101 a, 101b, 101c, 101d, 10e comprises a first fluid chamber 182a′,182b′,182c′,182d′,182e′ and a second fluid chamber 182a″,182b″,182c″,182d″,182e″. Each member 101a, 101b, 101c, 101d, 101e can be bent in a first direction relative to the main portion M by the removal of fluid from the first chambers 182a′,182b′,182c′,182d′,182e′, and bent in a second direction relative to the main portion M by the removal of fluid from the second chambers 182a″,182b″,182c″,182d″,182e″.

In the state shown in FIG. 25, the rotating valve 180 is positioned such that the fluid conduit 181 connects the first chamber 182c′ of the third member 101c to the second chamber 182d″ of the fourth member 101d. The conduit 181 is further connected to a central conduit, which in turn is connected to a hydraulic pump (not shown), such as the hydraulic pumps described with reference to FIGS. 54a-58. In the state shown in FIG. 25, the hydraulic pump has sucked hydraulic fluid from the first chamber 182c′ of the third member 101c, causing the third member to bend in a direction towards the stomach engager portion 151b of the second member 101b. Further, the hydraulic pump has sucked hydraulic fluid from the second chamber 182d″ of the fourth member 101d, causing the fourth member to bend in a direction towards the stomach engager portion 151e of the fifth member 101e. As such, the third and fourth members 101c, 101d bends in directions away from each other causing the portion of the stomach to which the respective third and fourth stomach engager portion 151c, 151d are fixated to move away from each other creating a stretching between the third and fourth members 101c, 101d.

The rotation of the rotating valve 180 causes two new pairs of chambers to be connected and in this way the medical device sequentially stretches five different portions of the stomach wall such that satiety can be created without inducing fatigue or loss of sensitivity to stretching at a specific portion of stomach wall.

When the rotating valve 180 has completed a ⅕ revolution from the state in which the valve is in FIG. 25, the fourth and fifth members 101d, 101e are simultaneously bent, such that the stomach engager portion 151d of the fourth member 101d moves towards the stomach engager portion 151c of the third member 101c and the stomach engager portion 151e of the fifth member 101e moves towards the stomach engager portion 151a of the first member 101a. As such, the fourth and fifth members 101d, 101e bends in directions away from each other causing the portion of the stomach to which the respective fourth and fifth stomach engager portion 151d, 151e are fixated to move away from each other creating a stretching between the fourth and fifth members 101d, 101e. As the rotating valve 180 rotates ⅕ revolution at a time, all the members are bent in pairs creating sequentially stretching of the five different portions of the stomach wall.

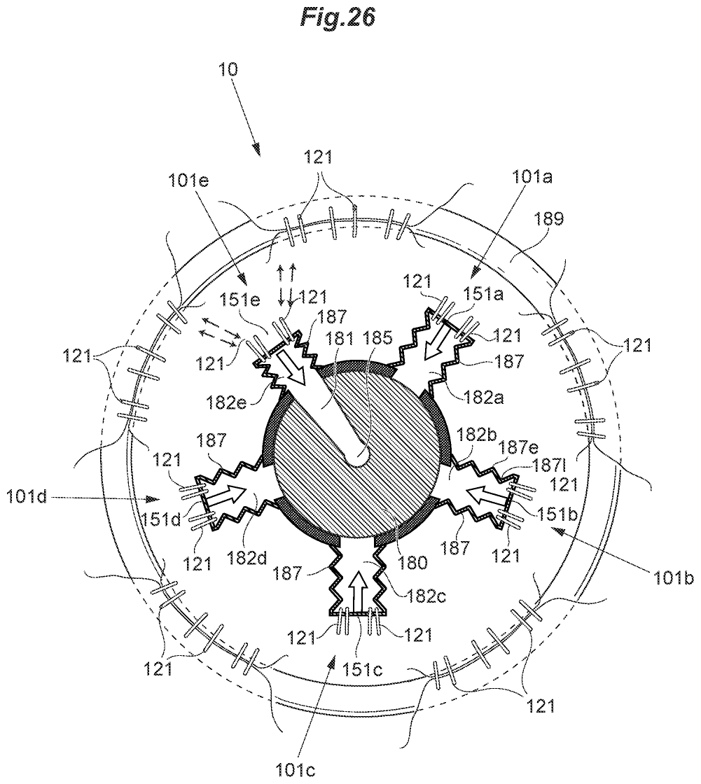

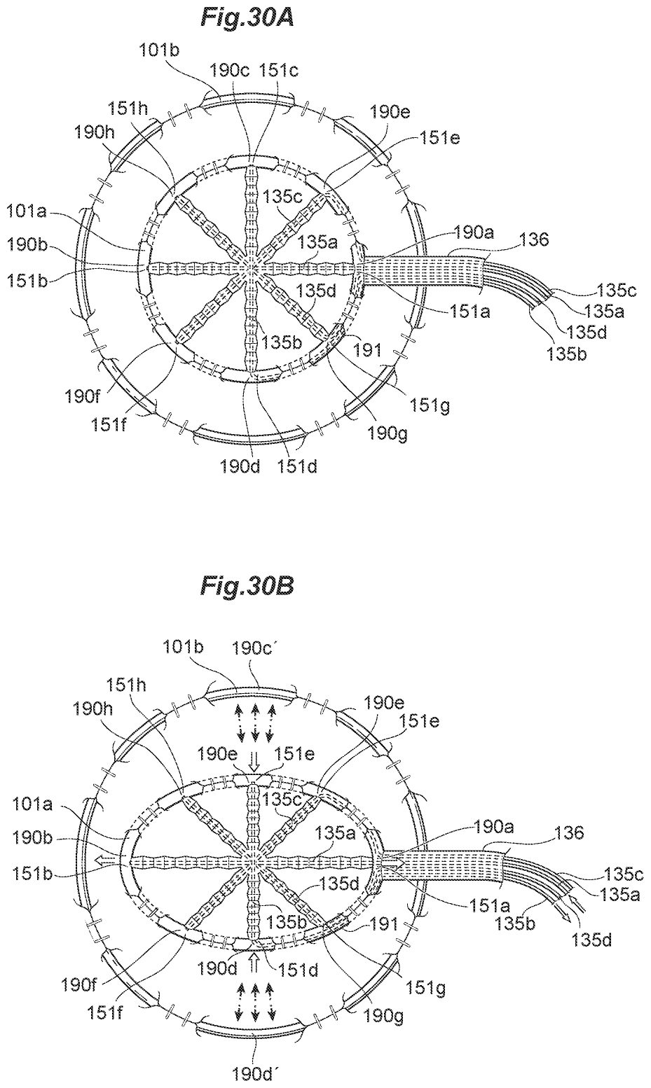

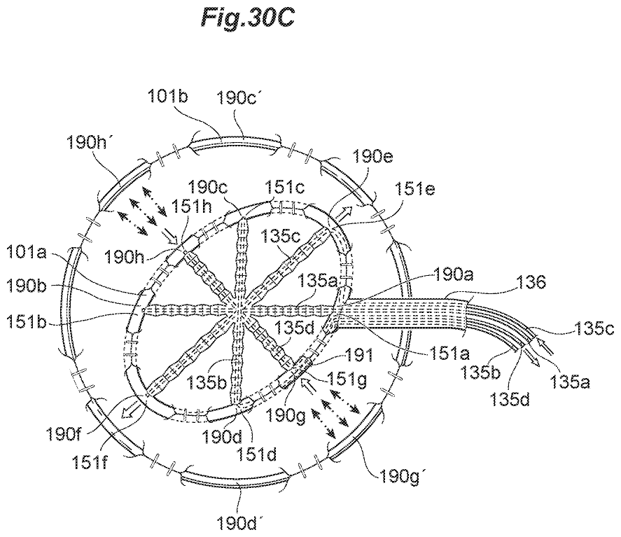

FIG. 26 shows an embodiment of the medical device similar to that of the embodiment of FIG. 24. The difference being that in the embodiment if FIG. 26, the first—fifth members 101a-101e are not connected to a pivoting element. As such, compression and expansion of the members 101a-101e only creates a linear movement in a radial direction in relation to the center 185 of the rotating valve 180. Just as in the embodiments described with reference to FIGS. 24 and 25, each member 101a-101e comprises a stomach engager 151a-151e fixated to the stomach wall. In the embodiment of FIG. 26, the medical device further comprises a simple closed curve in the form of a torus-shaped ring 189 configured to encircle a portion of the stomach wall of the patient. The ring 189 acts in the same way as the second member of the embodiment of FIGS. 30a-31b and is configured to act as a support against the force created by the operation of the first—fifth members 101a-101e, such that the stomach wall between the first—fifth members 101a-101e and the ring 189 can be stretched. Just as the first member 101a, the ring 189 in the embodiment shown in FIG. 26 is configured to be fixated to the stomach wall of the patient by means of invagination using stomach-to-stomach sutures or staplers. However, the ring could in alternative embodiments be configured to be fixated to the stomach wall using sutures and/or staplers, or by means of a tissue growth promoting structure, such that the stomach engagers can be fixated to the stomach wall by in-growth of fibrotic tissue. The ring 189 is in the embodiment shown in FIG. 26 substantially rigid with a major portion of the ring 189 being made from a material having a modulus of elasticity in the range 0.2 GPa-1000 GPa or in the range 1 GPa-400 GPa, such that the second member has a modulus of elasticity in the range 0.2 GPa-1000 GPa or in the range 1 GPa-400 GPa.

In the state shown in FIG. 26, the rotating valve 180 is positioned such that the fluid conduit 181 is connected to the fluid chamber 182e of the fifth member 101e. The conduit 181 is further connected to a central conduit, which in turn is connected to a hydraulic pump (not shown), such as the hydraulic pumps described with reference to FIGS. 54a-58. In the state shown in FIG. 26 the hydraulic pump has sucked hydraulic fluid from the chamber 182e of the fifth member 101e, causing the fifth member to compress and move the stomach engager portion 151e of the fifth member 101e radially in a proximal direction towards the center 185 of the rotating valve 180, and away from the ring 189 creating a stretching of the stomach wall placed between the fifth members 101e and the ring 189.

The rotation of the rotating valve 180 causes a new chamber to be connected to the fluid conduit 181 and thereby being operated for compression. In this way the medical device sequentially stretches five different portions of the stomach wall such that satiety can be created without inducing fatigue or loss of sensitivity to stretching at a specific portion of stomach wall.

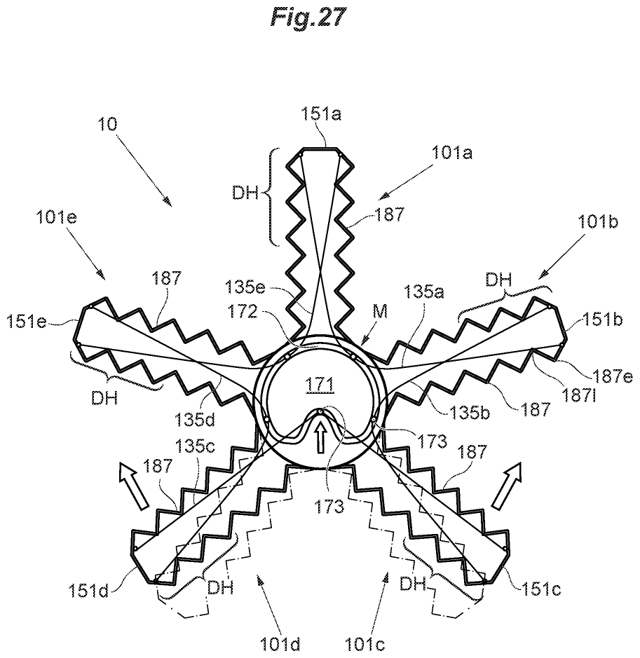

FIG. 27 shows an embodiment of the medical device for actively stretching a stomach wall of a patient according to an embodiment similar to the embodiments described with reference to FIGS. 24-26. The difference being that in the embodiment of FIG. 27, the bending of the members 101a-101e are created by flexible cables 135a-135e fixated to the respective member 101a-101e at the distal half DH of thereof. In the embodiment shown in FIG. 27, a first flexible cable 135a is connected to the first and second members 151a, 151b, at the distal half DH thereof, a second flexible cable 135b is connected to the second and third members 101b, 101c, at the distal half DH thereof, a third flexible cable 135c is connected to the third and fourth members 101c, 101d, at the distal half DH thereof, a fourth flexible cable 135d is connected to the fourth and fifth members 101d, 101e, at the distal half DH thereof, and a fifth flexible cable 135e is connected to the fifth and first members 101e, 101a, at the distal half DH thereof. Each flexible cable 135a-135e is fixated to a cylinder-shaped protrusion 173 and operated by a rotating eccentric operation device 171 comprising an eccentric rotatable engagement member in the form of a recess being a groove 172 encircling the center of rotation of the rotating eccentric operation device 171. Each cylinder-shaped protrusion 173 is configured to engage and run in the groove. In the state shown in FIG. 27, the eccentric portion of the groove 172 causes the cylinder-shaped protrusion 173 to move closed to the center of the rotating eccentric operation device 171, pulling on the third cable 135c, being fixated to the third and fourth members 101c, 101d and causing the third and fourth members 101c, 101d to move away from each other, which in turn causes a stretching of the stomach wall between a third and fourth portion of the stomach wall, to which the third and fourth members are fixated.

When the rotating eccentric operation device 171 rotates ⅕ of a revolution in a clock-wise direction, the eccentric portion of the groove 172 instead engages the cylinder-shaped protrusion 173 fixated to the fourth cable and causes the cylinder-shaped protrusion 173 fixated to the fourth cable 135d to move closer to the center of the rotating eccentric operation device 171, pulling on the fourth cable 135d, being fixated to the fourth and fifth members 101d, 101e and causing the fourth and fifth members 101d, 101e to move away from each other, which in turn causes a stretching of the stomach wall between a fourth and fifth portion of the stomach wall, to which the fourth and fifth members are fixated.

In the same way, the rotating eccentric operation device 171 engages and pulls on all the wires as it rotates, causing a sequential stretching of five portions of the stomach wall.

In the embodiment shown in FIG. 27, the members 101a-101e are bent or pivoted in a first direction by one cable and in a second direction by another cable. However, in alternative embodiments it is equally conceivable that the members 101a-101e are bent or pivoted in a first direction by one cable and in a second direction by an elastic element.

In the embodiment shown in FIG. 27, the members 101a-101e comprise flexible wall portions 187, comprising pleated portions P having elevated and lowered portions 187e,1871 enabling the members to bend or pivot also when fibrotic tissue is covering the elevated and lowered portions 187e,1871. The flexible cables 135a-135e are partially enclosed by the flexible wall portions. In the embodiment shown in FIG. 27, the flexible wall portions causes the members 135a-135e to bend or pivot in the desired direction when pulled on by the cables 135a-135e. The distance between the “peaks” of two elevated portions may be in the interval 2 mm-6 mm, or in the interval 2 mm-5 mm and the elevation difference between the lowest point of a lowered portion to a peak may be in the interval 1 mm-6 mm, or in the interval 1 mm-5 mm.

In the embodiment shown in FIG. 27, each member 101a-101e comprises a stomach engager portion 151a-151e being the distal portion of the members. In the embodiment shown in FIG. 27, the stomach engager portions are configured to be invaginated by the stomach wall using stomach-to-stomach sutures or staplers. However, in alternative embodiments, the stomach engager portions 151a-151e may comprise a fixation portion configured for fixation of the stomach engager portion to tissue of the stomach wall using at least one of sutures and staplers and/or a tissue growth promoting structure such that the stomach engager portion can be fixated to the stomach wall by in-growth of fibrotic tissue.

In the embodiment of FIG. 27, the medical device comprises a brushless direct current electrical motor (nor shown) connected to the rotating eccentric operation device 171 via a gear system configured to reduce the speed and increase the force of the movement generated by the electrical motor.

The medical device further comprises an energy storage unit for energizing the medical device.

In alternative embodiments, the operation device is partially positioned at a remote location in the body of the patient, in relation to the main portion M. A remote location may be a location being more than 5 cm from the main portion M, or more than 10 cm from the main portion M.

In the embodiment of FIG. 27, the medical device further comprises a controller for controlling the operation device. The controller comprises a wireless transceiver for communicating wirelessly with a source external to the body of the patient. The controller could be the controller (300) described in an of the embodiments with reference to FIGS. 54a-58.

The controller may comprise at least one sensor, or be configured to receive sensor input from the at least one sensor.

The sensor could be a sensor configured to sense a physical parameter of the medical device system, such as a temperature of the medical device system, to avoid excessive heating of tissue connected to the medical device during operation of the medical device or charging of the energy storage unit. Excessive heating may also damage the medical device and/or the energy storage unit. Excessive heating may also be an indicator that something is wrong with the medical device 10 and may be used for triggering an alarm function for alerting the patient or physician.

The sensor could be a sensor configured to sense a physical parameter of the medical device system, such as a parameter related to the power consumption of the medical device 10, to avoid excessive power consumption which may drain and/or damage the energy storage unit. Excessive power consumption may also be an indicator that something is wrong with the medical device 10 and may be used for triggering an alarm function for alerting the patient or physician.

The sensor could be a sensor configured to sense a physical parameter of the medical device system, such as a parameter related to strain in the medical device 10, such as the strain in the operation device or the members 101a-101e related to the operation of the medical device 10. Strain can be measured to avoid excessive strain which may damage medical device 10 or the tissue of the patient. Excessive strain may also be an indicator that something is wrong with the medical device 10 and may be used for triggering an alarm function for alerting the patient or physician. It may be important to measure strain or pressure in or exerted by the medical device, as too high strain or pressure risks hampering the blood flow to the tissue of the stomach wall, which in the long term could lead to damage of the tissue and in the worst-case lead to necrosis.

The sensor could be a sensor configured to sense a physical parameter of the medical device system, such as a parameter related to the patient swallowing, such that the medical device can be controlled on the basis of the patient eating or drinking. A sensor configured to sense a parameter related to the patient swallowing could comprises a motility sensor, which could be a piezo electric or piezo resistive motility sensor, or an accelerometer. In the alternative, a acoustic sensor, such as a microphone, may be used to sense the patient swallowing by picking up the sound generated by the patient swallowing. In the alternative, an optical sensor may be used for sensing the opacity alteration over the esophagus as food passes. A strain sensor could also be used for sensing the expansion of the esophagus as food passes. An accelerometer could be an accelerometer configured to measure acceleration in one plane, two planes or three planes, such that movement such as vibrations of e.g. the sternum can be measured in one, two or three axis, which can be used to separate the swallowing of solid foods from the swallowing of fluids, and separate the speech from swallowing and movement from swallowing. For this purpose, a controller of the medical implant, or a controller external to the body of the patient could use input from the sensor, such as the accelerometer and run through a filtering unit in which of the internal or external controller, such that motion patterns linked to speech and movement can be filtered from motion patterns being linked to swallowing, and the swallowing of fluids can be distinguished from the swallowing of solids. The features and functions of sensors sensing the patient swallowing is further described with reference to FIG. 16.

The sensor could be a sensor configured to sense a physical parameter of the medical device system, such as a local temperature, to avoid local excessive heating which may damage tissue of the patient or a systemic temperature, to avoid systemic excessive heating which may cause fever and affect the overall wellbeing of the patient.

The sensor could be a sensor configured to sense a physical parameter of the medical device system, such as a parameter related to an ischemia marker such as lactate, to control and/or avoid that the flow of blood to some tissue portion is hampered by the implantation or operation of the medical device 10. Hampered blood flow may lead to tissue damage and in the worst cases to tissue necrosis.

The sensor could be a sensor configured to sense a physical parameter of the medical device system, such as a blood pressure, which may be an indication that the strain created by the medical device is in some way damaging to the overall wellbeing of the patient. Increased blood pressure may be used for triggering an alarm function for alerting the patient or physician.

The sensor could be a sensor configured to sense a physical parameter of the medical device system, such as a pH, for determining the acidity of the stomach, which could be an indicator of the function of the digestive system and/or of the frequency of ingestion. The pH may be used for controlling the medical device 10 on the basis of the patient eating or drinking.

The controller may comprise a transmitter configured to transmit signals containing information to a receiver located exterior to the body of the patient. The transmitter could be a wireless transmitter configured to communicate with a wireless receiver located external to the body of the patient. In the embodiment shown in FIG. 27, the wireless transmitter of the controller is configured to transmit information based on sensor input to a source external to the body of the patient.

The controller may further comprise a receiver for receiving patient generated control signals from a unit located external to the body of the patient. The receiver could be a wireless receiver configured to communicate with a transmitter located external to the body of the patient. The controller may be configured to control the operation device on the basis of the received patient generated control signal. The control signal could for example being that the patient indicates to the medical device 10 that the patient has finished a portion of food which causes the medical device 10 to operate to stretch the stomach wall of the patient such that satiety is created which reduces the patients willing to eat further. In the alternative, the controller 300 may be configured to control the operation device 100 on the basis of a signal related to a lapsed time or a time of day such that satiety can be created with certain intervals or during specific periods of the day. The controller 300 could further be configured to receive a signal from a sensor external to the body of the patient and use such signal for controlling the operation of the medical device 10. The sensor external to the body of the patient could be a sensor could be a sensor measuring a parameter related to the patient eating to create input for the control of the medical device 10. Such a parameter could be related to body temperature, blood pressure or the glucose level of the blood. In the alternative, the sensor could be a sensor sensing a parameter related to the external environment, such as the atmospheric pressure, which could affect the pressures in the medical device 10.

In alternative embodiments, the bending or movement of the members (101a-101e) in any of the embodiment disclosed with reference to FIGS. 1A-27 could be propelled by a memory metal or an electroactive polymer which contracts or expands when stimulated by an electric field, such as when a voltage placed over the memory metal or electroactive polymer or a current is run through the memory metal or electroactive polymer fixated to or integrated in the respective member 101a-101e.

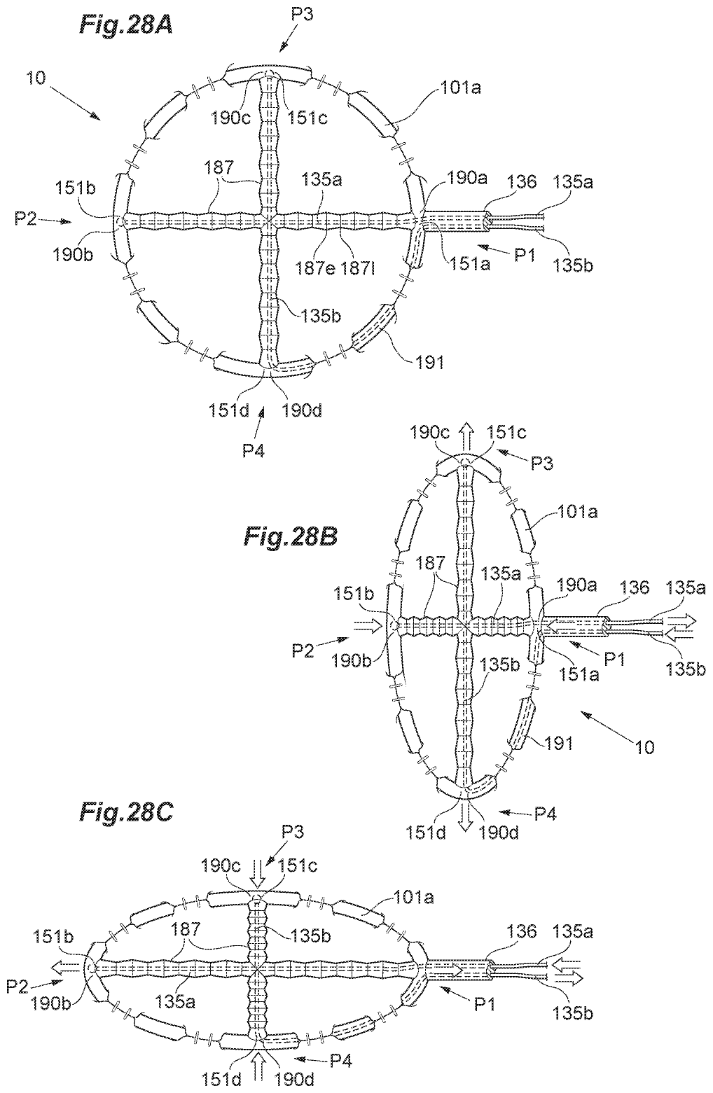

FIGS. 28a-28c shows an embodiment of the medical device 10 for actively stretching a stomach wall of a patient for creating a sensation of satiety. In the embodiment of FIGS. 28a-28c, the medical device comprises a first member 101a comprising a simple closed curve in the form of a flexible, elastic torus-shaped ring configured to encircle a portion of the stomach wall of the patient. The first member 101a comprises a first portion 190a comprising a first stomach engager 151a configured to engage a first portion P1 of the stomach wall and a second portion 190b comprising a second stomach engager 151b configured to engage a second portion P2 of the stomach wall, wherein the first member 101a is operable such that the first and second portions 190a,190b, comprising the first and second stomach engagers 151a, 151b, can move towards each other or away from each other for stretching a portion of the stomach wall. FIG. 28a shows the medical device 10 according to the embodiment of FIGS. 28a-28c in its idle state. In the embodiment shown in FIGS. 28a-28c, the first member 101a of the medical device 10 is mechanically operable by means of a mechanical operation device comprising flexible elements 135a,135b, fixated to the first and second portions 190a,190b of the first member 101a.

The first flexible element 135a is configured to operate, by the first flexible element being pulled, the first member 101a for moving the first portion 190a towards the second portion 190b, such that the third portion 190c moves away from the fourth portion 190d, for stretching the stomach wall between the third and fourth portions 190c,190d for creating a sensation of satiety. The second flexible element 135b is configured to operate, by the second flexible 135b element being pulled, the first member 101a for moving the first portion 190a towards the second portion 190b, such that the third portion 190c moves away from the fourth portion 190d, for stretching the stomach wall between the third and fourth portions 190c,190d for creating a sensation of satiety.

In the embodiment shown in FIGS. 28a-28c, the first member is mechanically operable from a remote unit, placed at a remote location in the body of the patient (further described with reference to FIG. 32). The remote unit transfers mechanical force from the remote unit to the first member 101a by means of the flexible wires 135a,135b. In the embodiment of FIGS. 28a-28c, the flexible wires 135a,135b comprises wire made up of a plurality of strands making the wire flexible but with relatively high torsional stiffness. The flexible wires 135a,135b are enclosed by a flexible protective cover 136 which encloses and protects the flexible shafts 135a,135b from the environment in the body. The protective cover 136 comprises an inner sleeve for reducing the friction between the flexible wires 135a,135b and the protective cover 136. Next the protective cover 136 comprises a structure providing support and protection and being configured to manage some level of external compression without that affecting the movement of the flexible shafts 135a,135b inside of the protective cover 136. The outermost layer of the protective cover 136 is a protective cover for sealing the flexible wires 135a,135b against the environment in the body of the patient. The protective cover 136 comprises a biocompatible flexible material, such as a biocompatible flexible polymer material, such as a silicone-based material, or a polyurethane-based material.

In the embodiment shown in FIGS. 28a-28c, the first member 101a comprises an integrated channel 191, thought which the second flexible element 135b travels from the protective cover 136 to the fourth portion 190d. At the fourth portion 190d, the second flexible wire 135b exits the first member 101a and travels across the diameter of the first member 101a and is fixated at the third portion 190c.

In the embodiment shown in FIGS. 28a-28c, the first and second flexible elements 135a,135b travels through a flexible sheath 187 covering and protecting the flexible wires 135a,135b.

The flexible sheath 187 comprises elevated and lowered portions 187e,1871, such that the flexible sheath 187 can remain flexible even as fibrotic tissue overgrows the flexible sheath 187. The flexible sheath 187 has the form of a pleated tube or bellows and comprises a biocompatible flexible material, such as a biocompatible flexible polymer material, such as a silicone-based material, or a polyurethane-based material.

In the embodiment shown in FIGS. 28a-28c, the first member 101a is configured to be invaginated by the stomach wall using stomach-to-stomach sutures or staplers. The invagination fixates the stomach engagers 151a-151d to the stomach wall, such that the stomach wall between the stomach engagers 151a-151d can be stretched for creating a sensation of satiety. In alternative embodiments, the stomach engagers could comprise at least one of fixation portions configured for fixation of the stomach engagers to tissue of the stomach wall using sutures and/or staplers, or the stomach engagers could comprise tissue growth promoting structure, such that the stomach engagers can be fixated to the stomach wall by in-growth of fibrotic tissue.

FIG. 28b shows the medical device 10 in a state in which the first member 101a has been operated by the first flexible wire 135a such that the first and second portions 190a,190b of the first member 101a have been moved towards each other, which has caused the third and fourth portions 190c,190d to move away from each other causing a stretching of the stomach wall placed between the third and fourth portions 190c,190d. When the first and second portions 190a,190b of the first member 101a have been moved towards each other the first member assumes an oval and elliptical shape. An oval shape is to be understood as a shape being a smooth-looking, simple closed curve being continuously convex and having a shape not departing much from that of an ellipse.

FIG. 28C shows the medical device 10 in a state in which the first member 101a has been operated by the second flexible wire 135b such that the third and fourth portions 190c,190d of the first member 101a have been moved towards each other, which has caused the first and second portions 190a,190b to move away from each other causing a stretching of the stomach wall placed between the first and second portions 190a,190b. When the third and fourth portions 190c,190d of the first member 101a have been moved towards each other the first member 101a assumes an oval and elliptical shape having the long axis perpendicular to the long axis of the elliptical shape formed by the first member 101a in FIG. 28B.

FIGS. 29A-29C shows an embodiment of the medical device 10 similar to the embodiment shown in FIGS. 28A—28C. The difference being that in the embodiment of FIGS. 29A—29C, the first member 101a is hydraulically operable by the medical device comprising a hydraulic operation device. In the embodiment of FIGS. 29A—29C, the medical device 10 comprises a hydraulic actuator 143, in the form of a hydraulic cylinder 143. The hydraulic cylinder 143 is fixated to the first and second portion 190a,190b of the first member 101a and is configured to operate the first member 101a for moving the first portion 190a towards the second portion 190b, and for moving the first portion 190a away from the second portion 190b. In the embodiment of FIGS. 29A—29C, a single hydraulic actuator 143 can create stretching of the stomach wall both between the first and second portions P1, P2 of stomach wall and of the stomach wall between the third and fourth portions P3, P4 of stomach wall.

In an alternative embodiment, the medical device may comprise a second hydraulic actuator, directly or indirectly fixated to the third and fourth portion of the first member, and being configured to operate the first member for moving the third portion towards the fourth portion and/or for moving the third portion away from the fourth portion.

In the embodiment shown in FIGS. 29a-29c, the hydraulic actuator 143 is connected to a hydraulic conduit 109 for conducting a hydraulic fluid to the hydraulic actuator 143. The first member 101a comprises an integrated channel 191′ for directly allowing the hydraulic fluid to be conducted by the integrated channel 191′, or for allowing the hydraulic conduit 109 to travel through the integrated channel 191′.

In the embodiment shown in FIGS. 29A—29C, the hydraulic cylinder comprises a flexible sheath 187 covering and protecting the hydraulic piston 143p. The flexible sheath 187 comprises elevated and lowered portions 187e,1871, such that the flexible sheath 187 can remain flexible even as fibrotic tissue overgrows the flexible sheath 187. The flexible sheath 187 has the form of a pleated tube or bellows and comprises a biocompatible flexible material, such as a biocompatible flexible polymer material, such as a silicone-based material, or a polyurethane-based material. The distance between the “peaks” of two elevated portions may be in the interval 2 mm-6 mm, or in the interval 2 mm-5 mm and the elevation difference between the lowest point of a lowered portion to a peak may be in the interval 1 mm-6 mm, or in the interval 1 mm-5 mm.

FIG. 29B shows the medical device 10 in a state in which the first member 101a has been operated by the hydraulic cylinder 143 such that the first and second portions 190a,190b of the first member 101a have been moved towards each other, which has caused the third and fourth portions 190c,190d to move away from each other causing a stretching of the stomach wall placed between the third and fourth portions 190c,190d. When the first and second portions 190a,190b of the first member 101a have been moved towards each other the first member assumes an oval and elliptical shape.

FIG. 29C shows the medical device 10 in a state in which the first member 101a has been operated by hydraulic cylinder 143 such that the first and second portions 190a,190b of the first member 101a have been moved away from each other, causing a stretching of the stomach wall placed between the first and second portions 190a,190b. When the first and second portions 190a,190b of the first member 101a have been moved away from each other, the first member 101a assumes an oval and elliptical shape having the long axis perpendicular to the long axis of the elliptical shape formed by the first member 101a in FIG. 29B.

FIGS. 30A-30C shows the medical device in an embodiment in which the medical device comprises a first and second member 101a, 101b. The first member 101a is similar to the first member 101a of the embodiment of FIGS. 28A—28C, the difference being that the first member 101a further comprises a third flexible element 135c fixated to a fifth and sixth portion 190e,190f of the first member 101a, and a fourth flexible element 135d fixated to a seventh and eight portion 190g,190h of the first member 101a. Operation of the third flexible wire 135c causes the fifth and sixth portions 190e,190f of the first member 101a to move towards each other, which causes the seventh and eight portions 190g,190h to move away from each other and operation of the fourth flexible wire 135d causes the seventh and eight portions 190g,190h of the first member 101a to move towards each other, which causes the fifth and sixth portions 190e,190f to move away from each other.

The second member 101b is configured to act as a support against the force created by the operation of the first member 101a, such that the stomach wall between the first and second members 101a, 101b can be stretched. Just as the first member 101a, the second member 101b is in the embodiment shown in FIGS. 30a-30c configured to be fixated to the stomach wall of the patient by means of invagination using stomach-to-stomach sutures or staplers. However, also the second member 101b could in alternative embodiments be configured to be fixated to the stomach wall using sutures and/or staplers, or by means of a tissue growth promoting structure, such that the stomach engagers can be fixated to the stomach wall by in-growth of fibrotic tissue.

Just as the first member 101a, the second member 101b is a simple closed curve in the form of a torus-shaped ring configured to encircle a portion of the stomach wall of the patient. The difference is that the first member 101a is more flexible and more elastic than the second member 101b. The second member 101b is in the embodiment shown in FIGS. 30a-30c substantially rigid with a major portion of the second member being made from a material having a modulus of elasticity in the range 0.2 GPa-1000 GPa or in the range 1 GPa-400 GPa, such that the second member has a modulus of elasticity in the range 0.2 GPa-1000 GPa or in the range 1 GPa-400 GPa. A major portion of the second member 101b could for example comprise a biocompatible metallic material, such as titanium or a medical grade metal alloy, such as medical grade stainless steel. In the alternative, the second member 101b could comprise a ceramic material such as zirconium carbide, or a stiff medical grade polymer material such as Ultra-high-molecular-weight polyethylene (UHMWPE) or Polytetrafluoroethylene (PTFE) or a thermoplastic polyester such as polylactide (PLA). The second member 101b could also comprise at least one composite material, such as any combination of metallic/ceramic and polymer materials or a polymer material reinforced with organic or inorganic fibers, such as carbon or mineral fibers.

FIG. 30B shows the medical device 10 in a state in which the first member 101a has been operated by the second flexible element 135b such that the third and fourth portions 190c,190d of the first member 101a have been moved towards each other. The movement of the third and fourth portions 190c,190d towards each other causes the distance between the third portion 190c and a third outer portion 190c′ to increase, which causes a stretch of the stomach wall placed between the third portion 190c and the third outer portion 190c′. The movement of the third and fourth portions 190c,190d towards each other further causes the distance between the fourth portion 190d and a fourth outer portion 190d′ to increase, which causes a stretch of the stomach wall placed between the fourth portion 190d and the fourth outer portion 190d′. In the embodiment shown in FIG. 30B, the third and fourth portions 190c,190d moving towards each other further causes the distance between the first and second portions 190a,190b to increase, which causes a stretch of the stomach wall placed between the first and second portions 190a,190b. In this way, a simultaneous stretch of three portions of stomach wall is created.

FIG. 30C shows the medical device 10 in a state in which the first member 101a has been operated by the fourth flexible element 135d such that the seventh and eight portions 190g,190h of the first member 101a have been moved towards each other. The movement of the seventh and eighth portions 190g,190h towards each other causes the distance between the seventh portion 190g and a seventh outer portion 190g′ to increase, which causes a stretch of the stomach wall placed between the seventh portion 190g and the seventh outer portion 190g′. The movement of the seventh and eight portions 190g,190h towards each other further causes the distance between the eight portion 190h and an eight outer portion 190h′ to increase, which causes a stretch of the stomach wall placed between the eight portion 190h and the eight outer portion 190h′. In the embodiment shown in FIG. 30C, the seventh and eight portions 190g,190h towards each other further causes the distance between the fifth and sixth portions 190e,190f to increase, which causes a stretch of the stomach wall placed between the fifth and sixth portions 190e,190f. In this way, I simultaneous stretch of three portions of stomach wall is created.

FIGS. 31A and 31B shows an embodiment of the medical device similar to the embodiment shown in FIGS. 30a-30c. The difference being that a first portion 135a′ of the first flexible wire 135a connects the first portion 190a of the first member 101a to a first outer portion 190a′ of the second member 101b, and a second portion 135a″ of the first flexible wire 135a connects the second portion 190b of the first member 101a to a second outer portion 190b′ of the second member 101b. A first portion 135b′ of the second flexible wire 135b connects the third portion 190c of the first member 101a to the third outer portion 190c′ of the second member 101b, and a second portion 135b″ of the second flexible wire 135b connects the fourth portion 190d of the first member 101a to the fourth outer portion 190d′ of the second member 101b. The second member comprises channels 191 in which the flexible wires can travel, such that the flexible wires 135a,135b can be bundled in the flexible protective cover 136.

The first and second portions 135a′,135a″ of the first flexible wire 135a are connected at a first connection point 135ac in the flexible protective cover 136, and the first and second portions 135b′,135b″ of the second flexible wire 135b are connected at a second connection point 135bc in the flexible protective cover 136.

When the first flexible wire 135a is pulled, the first portion 135a′ of the first flexible wire 135a pulls on the first portion 190a of the first member 101a and the second portion 135a″ of the first flexible wire 135a simultaneously pulls on the second portion 190b of the first member 101a, which moves the first portion 190a of the first member 101a closer to the first outer portion 190a′ and the second portion 190b of the first member 101a closer to the second outer portion 190b′, of the second member 101b.

When the second flexible wire 135b is pulled, the first portion 135b′ of the second flexible wire 135b pulls on the third portion 190c of the first member 101a and the second portion 135b″ of the second flexible wire 135b simultaneously pulls on the fourth portion 190d of the first member 101a, which moves the third portion 190c of the first member 101a closer to the third outer portion 190c′ and the fourth portion 190d of the first member 101a closer to the fourth outer portion 190d′, of the second member 101b.

FIG. 31B shows the medical device 10 in a state in which the first member 101a has been operated by the first flexible element 135a such that the first and second portions 190a,190b of the first member 101a have been moved away from each other. The movement of the first and second portions 190a,190b away from each other causes a stretch of the stomach wall placed between the first and second portions 190a,190b. In addition, in the embodiment shown in FIG. 31b, the movement of the first and second portions 190a,190b away from each other causes the third and fourth portions 190c,190d of the first member 101a to move towards each other (as the first member 101a assumes an elliptical shape), which causes a stretch of the stomach wall between the third portion 190c of the first member 101a and the third outer portion 190c′ of the second member 101b, and between the fourth portion 190d and the fourth outer portion 190d′ of the second member 101b.

FIG. 31C shows an embodiment of the medical device 10 similar to the embodiment shown in FIGS. 31A and 31B. The difference being that in the embodiment shown in FIG. 31C the first member 101a is configured to be moved relative to the second member for stretching the tissue of the stomach wall placed between the first and second members. In the embodiment shown in FIG. 31C, the operation device comprises three flexible wires 135a, 135b, 135c. The first flexible wire connects the first portion 190a of the first member 101a to a first outer portion 190a′ of the second member 101b, the second flexible wire 135b connects the second portion 190b of the first member 101a to a second outer portion 190b′ of the second member 101b, and the third flexible wire 135c connects the third portion 190c of the first member 101a to the third outer portion 190c′ of the second member 101b. The second member 101b comprises channels 191 in which the flexible wires can travel, such that the flexible wires 135a, 135b, 135c can be bundled in the flexible protective cover 136. Pulling on the first flexible wires moves the first member 101a in relation to the second member such that the first portion 190a of the first member 101a is moved closer to the first portion 190a′ of the second member, such that stretching of the tissue of the stomach wall is created between a fourth portion 190d of the first member and a fourth portion 190d′ of the second member 101b. As the medical device of the embodiment shown in FIG. 31C operates in the same way when the second and third flexible wires 135b, 135c are moved, the embodiment of FIG. 31C enables stretching of three main regions of tissue of the stomach wall between the first and second members 101a, 101b.

FIGS. 31D and 31E shows an embodiment of the medical device 10 similar to the embodiments shown in FIGS. 28A and 31C. The difference being that in the embodiment shown in FIGS. 31D and 31E, the first member 101a is a ring-shaped member forming a closed curve and is configured to be deformed by pulling action from flexible wires 135a, 135b, 135c as shown in FIG. 31D. Each of the flexible wires are connected to a drum or spool 146a, 146b, 146c onto which the flexible wires 135a, 135b, 135c are configured to be wound. Each of the drums or spools 146a, 146b, 146c are operated by a propulsion device comprising electrical motors MO1, MO2, MO3 rotating the drums or spools 146a, 146b, 146c winding the flexible wires 135a, 135b, 135c which creates a pull on the flexible wires 135a, 135b, 135c. The flexible wires 135a, 135b, 135c are placed around pulleys 144a, 144b, 144c rotatably fixated to portions of the first member 101a. The drums or spools 146a, 146b, 146c and electrical motors MO1, MO2, MO3 are equidistantly positioned on the second member, and the pulleys 144a, 144b, 144c are equidistantly positioned on the first member 101a.

FIG. 31E shows the embodiment of the medical device of FIG. 31D, when in a state in which the second motor MO2 has operated the second drum or spool 146b such that the second flexible wire 135b has been wound on the second drum or spool 146b. The flexible wire 146b runs around a second pulley 144b and back to a fixation point close to the second drum or spool 146b. The set-up with the pulleys creates a 2:1 transmission, i.e. movement of 10 mm of the second flexible wire 135b creates a 5 mm movement of the second pulley 144b which moves a second portion 190b of the first member in a direction towards the second spool or drum 146b which deforms the first member 101a such that the second portion 190b of the first member 101a is moved away from the second portion 190b′ of the second member 101b such that the tissue of the stomach wall between the between the second portion 190b of the first member 101a and the second portion 190b′ of the second member 101b. 42. As the medical device of the embodiment shown in FIG. 31E operates in the same way when the first and third flexible wires 135a, 135c are moved, the embodiment of FIG. 31E enables stretching of three main regions of tissue of the stomach wall between the first and second members 101a, 101b. At least the portions 190a-190c of the first member 101a at which the pulleys 144a-144c are fixated (the portions between the positions that the stiff portions 197 of the sheaths are fixated) are flexible such that these portions 190a-190c can deform. In the embodiment shown in FIGS. 31D and 31E, the flexible portions 190a-190c comprises an elastic material in the form of an elastic polymer material such as a silicone or polyurethane-based material.

In the embodiment shown in FIGS. 31D and 31E, the flexible wires 135a, 135b, 135c runs in sheaths having a stiff portion 197 and a flexible portion 187. The stiff portion fixates portions of the first member 101a, thus ensuring that the first member 101a is fixed in a centered position in the second member 101b for ensuring that the pulling force exerted by the flexible members 135a-135c deforms the first member 101a instead of moving the first member 101a. The flexible portions 187 comprise pleated portions having elevated and lowered portions enabling the flexible portions to contract also when fibrotic tissue is covering the elevated and lowered portions. The distance between the “peaks” of two elevated portions may be in the interval 2 mm-6 mm, or in the interval 2 mm-5 mm and the elevation difference between the lowest point of a lowered portion to a peak may be in the interval 1 mm-6 mm, or in the interval 1 mm-5 mm.

In the embodiments shown in FIGS. 31D and 31E, the propulsion device comprising electrical motors MO1, MO2, MO3 are electrically connected to a controller for controlling the operation of the motors MO1, MO2, MO3 by means of electrical leads 474a, 474b, 474c. The controller may be placed locally, close to the first and second members 101a, 101b, or may be placed in a remote unit. Features of the remote unit and the controller are further described with reference to FIGS. 33-65f.

One advantage with the embodiment shown in FIGS. 31D and 31E is that the flexible wires 135a-135c are configured to stretch the stomach in a main stretching direction aligned with the direction of the pulling force of the flexible wires 135a-135c. This results in that most of the torque generated by the motor is translated in the pulling force that stretches the stomach wall which means that losses incurred by directional change of the pulling force is minimized.

FIG. 31F shows an embodiment of the medical device similar to that shown in FIG. 31C.

Just as in the embodiment shown in FIG. 31C, the first member 101a is moved in relation to the second member 101b for creating stretching between the first and second members 101a, 101b. The difference between the embodiment shown in FIG. 31F and the embodiment shown in FIG. 31C is that the operation device comprises an arm 199 having a joint 196. The arm is rotatably fixated to a driving unit 168. The arm 199 is fixedly fixated to the first member 101a and rotatably fixated to the driving unit 168. The first and second members 101a, 101b are further connected to each other by means of a flexible wire 135 running in a sheath having a flexible wall 187 with a pleated portion such that the sheath can be extended and compressed also after fibrotic tissue has grown in the flexible wall 187. The sheath is connected to the first and second members 101a, 101b and the flexible wire 135 runs through the sheath and enters the driving unit 168 and is wound on a drum or spool 146 connected to a propulsion device comprising a first electrical motor MO1 for rotating the drum or spool 146 and thereby pulling or releasing the flexible wire 135. The pulling of the flexible wire 135 moves the first member 101a closer to the driving unit 168. The driving unit 168 encloses the first electrical motor MO1 and the drum or spool 146 and further encloses a second electrical motor MO2 operably connected to the rotatable fixation of the arm 199 by means of a bevel gear 198, such that operation of the second motor MO2 operates the arm 199 to move the first member 101a up and down in the view in FIG. 31F. The combination of rotation by the arm 199 and extension and shortening and elongation of the flexible wire 135 enables the first member 101a to be relatively freely moved within the second member 101b, such that the basically all different regions of tissue of the stomach wall between the first and second members 101a, 101b can be stretched.

The propulsion device comprising the electrical motors MO1, MO2 are electrically connected to a controller for controlling the operation of the motors MO1, MO2 by means of an electrical lead 474. The controller may be placed locally, close to the first and second members 101a, 101b, or may be placed in a remote unit. Features of the remote unit and the controller are further described with reference to FIGS. 33-65f.

FIG. 31G shows an embodiment of the medical device similar to that shown in FIG. 31F.

Just as in the embodiment shown in FIG. 31F, the first member 101a is moved in relation to the second member 101b for creating stretching of the tissue of the stomach wall between the first and second members 101a, 101b. The difference between the embodiment shown in FIG. 31G and the embodiment shown in FIG. 31F is that the operation device comprises three flexible wires 135a, 135b, 135c. The first flexible wire connects a first portion 190a of the first member 101a to a first portion 190a′ of the second member 101b. The second flexible wire 135b connects a second portion 190b of the first member 101a to a second portion 190b′ of the second member 101b and the third flexible wire 135c connects a third portion 190c of the first member 101a to a third portion 190c′ of the second member 101b.

Each of the flexible wires 135a, 135b, 135c are connected to a drum or spool 146a, 146b, 146c onto which the flexible wires 135a, 135b, 135c are configured to be wound. Each of the drums or spools 146a, 146b, 146c are operated by a propulsion device comprising electrical motors MO1, MO2, MO3 rotating the drums or spools 146a, 146b, 146c winding the flexible wires 135a, 135b, 135c which creates a pull on the flexible wires 135a, 135b, 135c. The flexible wires 135a, 135b, 135c are fixated at the first, second and third portions 190a, 190b, 190c of the first member 101a. The drums or spools 146a, 146b, 146c and electrical motors MO1, MO2, MO3 are equidistantly positioned on the second member 101b, and the flexible wires 135a, 135b, 135c are equidistantly fixated to the first member 101a. The flexible wires 135a, 135b, 135c runs in sheaths having a flexible wall 187 with a pleated portion such that the sheaths can be extended and compressed also after fibrotic tissue has grown in the flexible wall 187. The sheaths are connected to the first and second members 101a, 101b and the flexible wire 135 runs through the sheath and enters the second member 101b and is wound on the drums or spools 146a, 146b, 146c.

In FIG. 31G, the medical device is shown in a state in which the second motor MO2 has operated the second drum or spool 146b such that the second flexible wire 135b has been wound on the second drum or spool 146b which moves the second portion 190b of the first member 101a in a direction towards the second spool or drum 146b such that the tissue of the stomach wall between the between a fourth portion 190d of the first member 101a and a fourth portion 190d′ of the second member 101b is stretched. As the medical device of the embodiment shown in FIG. 31G operates in the same way when the first and third flexible wires 135a, 135c are moved, the embodiment of FIG. 31G enables stretching of three main regions of tissue of the stomach wall between the first and second members 101a, 101b.

In the embodiments shown in FIGS. 31D and 31E, the electrical motors MO1, MO2, MO3 are electrically connected to a controller for controlling the operation of the motors MO1, MO2, MO3 by means of electrical leads 474a, 474b, 474c. The controller may be placed locally, close to the first and second members 101a, 101b, or may be placed in a remote unit. Features of the remote unit and the controller are further described with reference to FIGS. 33-65f.

FIG. 31H shows an embodiment of the medical device similar to that shown in FIG. 31G.

Just as in the embodiment shown in FIG. 31G, the first member 101a is moved in relation to the second member 101b for creating stretching of the tissue of the stomach wall between the first and second members 101a, 101b. The difference between the embodiment shown in FIG. 31H and the embodiment shown in FIG. 31G is that the propulsion device comprising three electrical motors MO1, MO2, MO3 operating the flexible wires 135a, 135b, 135c are placed on one side of the second member 101b and the flexible wires 135a, 135b, 135c are connected to one side of the first member 101a. Just as in the embodiment shown in FIGS. 31D and 31E the flexible wires 135a, 135b, 135c are placed around pulleys 144a, 144b, 144c which are respectively rotatably fixated to the first, second and third portions 190a, 190b, 190c of the first member 101a.

FIG. 31H shows the medical device in a state in which the third motor MO3 has operated the third drum or spool 146c such that the third flexible wire 135c has been wound on the third drum or spool 146c. The flexible wire 146c runs around the third pulley 144c and back to a fixation point close to the third drum or spool 146c. The set-up with the pulleys creates a 2:1 transmission, i.e. movement of 10 mm of the third flexible wire 135c creates a 5 mm movement of the third pulley 144c which moves the first member 101a in a direction towards the third spool or drum 146c which increases the distance between the third portion 190c of the first member and the third portion 190c′ of the second member 101b, such that mainly the tissue of the stomach wall between the third second portion 190c of the first member 101a and the third portion 190c′ of the second member 101b is stretched. However, as also the distance between the second portion 190b of the first member 101a and the second portion 190b′ of the second member 101b is somewhat increased, some stretching also occurs of the tissue of the stomach wall placed between the second portion of the first member 101a and the second portion 190b′ of the second member 101b.

As the medical device of the embodiment shown in FIG. 31H operates in the same way when the first and second flexible wires 135a, 135b are moved, the embodiment of FIG. 31H enables stretching of three main regions of tissue of the stomach wall between the first and second members 101a, 101b as well as regions between these main regions by the operation of more than one electrical motor. I.e., by pulling differently on the flexible members 135a, 135b, 135c, the first member 101a can be moved in varying degree towards the three drums or spools 146a, 146b, 146c.

The flexible wires 135a, 135b, 135c runs in sheaths having a flexible walls 187 with pleated portions such that the sheaths can be extended and compressed also after fibrotic tissue has grown in the flexible wall 187. The sheaths are connected to the first and second members 101a, 101b and the flexible wires 135a, 135b, 135c runs through the sheaths and enters the second member 101b and is wound on the drums or spools 146a, 146b, 146c.

In the embodiments shown in FIG. 31H, the electrical motors MO1, MO2, MO3 are electrically connected to a controller for controlling the operation of the motors MO1, MO2, MO3 by means of electrical leads 474a, 474b, 474c. The controller may be placed locally, close to the first and second members 101a, 101b, or may be placed in a remote unit. Features of the remote unit and the controller are further described with reference to FIGS. 33-65FH.

FIGS. 31C, 31F, 31G, 31H and 31X-31X″′ are examples of embodiment in which the medical device comprises second members configured to be fixated to the stomach wall of the human and comprising a closed curve and first members configured to be fixated to the stomach wall of the human within the footprint of the second member. The first members are operably connected to the second members such that the first members can be displaced: relative to the second members, and within the footprint of the second members. Generally, a non-therapeutic method of creating a sensation of satiety using a pre-implanted medical device configured to stretch a portion of the stomach wall of a human is further provided. The non-therapeutic method generally comprises operating the first member for displacing the first member relative to the second member within the footprint of the second member, for stretching a stomach wall portion between the first and second member, for creating a sensation of satiety.

The non-therapeutic method of creating a feeling of satiety could be linked to a life-style choice of not eating too much or could be linked to a desired physical appearance and as such be linked to physical esthetics.

The medical devices of FIGS. 31C, 31F, 31G, 31H and 31X-31X″′ may comprise or be connected to an implantable controller configured to control the operation of the first member. In such embodiments, the non-therapeutic method comprises operating the first member as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

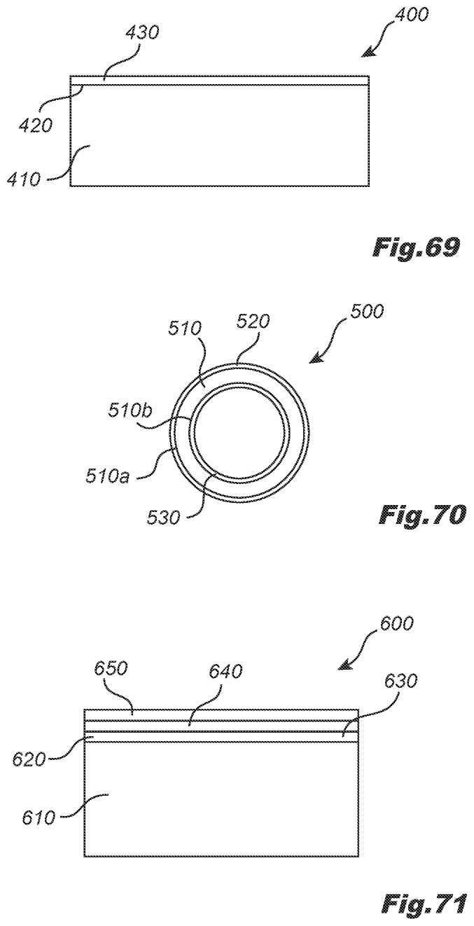

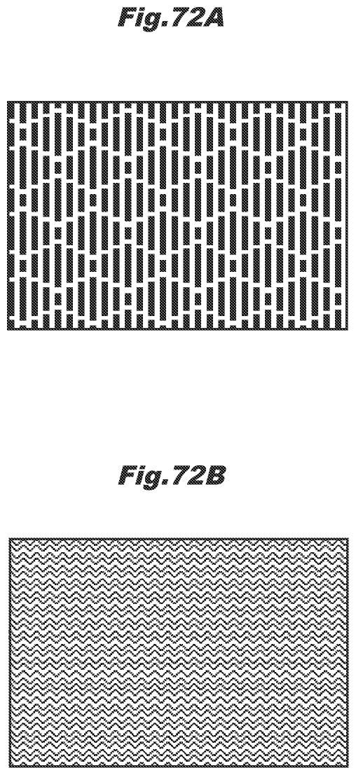

FIG. 31I shows an embodiment of the medical device in which the first member 101a is placed within the footprint of the second member 101b, without being connected to the second member 101b. The first member 101a is pleated, such that the first member can be deformed by means of flexible wires 135a, 135b running inside of the first member 101a. In the state shown in FIG. 311, the first flexible wire 135a has been pulled, such that the right side of the first member has been compressed. The compression of the right side of the first member leads to an increase in the distance between a first portion 190a of the first member 101a and a first portion 190a′ of the second member 101b, which stretches the tissue wall placed between the first portion 190a of the first member 101a and the first portion 190a′ of the second member 101b. The first and second flexible wires 135a, 135b are connected to each other and fixated to the first member at a fixation point 169. The first and second flexible wires 135a, 135b are wires of Bowden cable type, which are flexible but relatively stiff. As such, the second wire 135b supports the left side of the first member in the state shown in FIG. 31I, such that the left side does not deform when the first flexible wire 135a is pulled for deforming the right side of the first member 101a. In the same way, the first flexible wire supports the right side of the first member 101a when the second wire is pulled for deforming the left side of the first member 101a.

FIGS. 30A-30C, 31B, 31D, 31E, 31I, 31M-31P and 31U-31V′. are examples of embodiments in which the medical device comprises second members configured to be fixated to the stomach wall of the human and operable first members configured to be fixated to the stomach wall of the human, at least partially within the footprint of the second members. The first members are configured to be operated to deform. Generally, a non-therapeutic method of creating a sensation of satiety using a pre-implanted medical device configured to stretch a portion of the stomach wall of a human is further provided. The non-therapeutic method generally comprises operating the first members to deform, such that a first portion of the first members are moved away from a first portion of the second members, for stretching a portion of the stomach wall between the first portion of the first members and the first portion of the second members, for creating a sensation of satiety.

The non-therapeutic method of creating a feeling of satiety could be linked to a life-style choice of not eating too much or could be linked to a desired physical appearance and as such be linked to physical esthetics.

The medical devices of FIGS. 30A-30C, 31B, 31D, 31E, 31I, 31M-31P and 31U-31V′ may comprise or be connected to an implantable controller configured to control the operation of the first member. In such embodiments, the non-therapeutic method comprises operating the first member as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

FIGS. 31J-31L′ shows three versions of embodiments of the first or second member, the principle of which may be used in any of the embodiments shown with reference to FIGS. 28A-31I and 31M-31V′. In all of the embodiments shown in FIGS. 31J-31L′, the outer diameter of the ring-shaped member 101 can be adjusted, for calibrating the stretching performed by medical device. The calibration ensures that the tissue placed between a first and a second member is stretched to a suitable starting state before the medical device is operated for stretching the tissue wall. In the embodiment shown in FIG. 31J, the member 101 comprises an inflatable portion 175 encircling the outer periphery of the member 101.

The inflatable portion 175 is fluidly connected to a fluid conduit 109 which may connect the inflatable portion 175 to a subcutaneously placed injection port or to a hydraulic pump which may be placed in a remote unit. The inflatable portion 175 is configured to expand and thereby increase the outer diameter of the member 101.

In the embodiment shown in FIG. 31K, the member 101 comprises a flexible portion 187 comprising a pleated portion which can be expanded and contracted for altering the diameter of the member 101. In the embodiment shown in FIG. 31K, the member 101 is operated by a flexible wire 135 configured to transfer a rotating force. The flexible wire 135 comprises a threaded portion which engages a nut-portion having inner threads such that a nut-and-screw transmission element 138 is created for transferring the rotating force transferred by the flexible wire 135 to a linear force for expanding and contracting the flexible portion 187 to thereby increase and decrease the diameter of the member 101 to alter the idle tension that the member 101 creates on the tissue wall of the stomach before the medical device is operated for stretching the stomach wall to create an induced feeling of satiety.

FIG. 31K′ shows an embodiment of the member 101 similar to the embodiment shown in FIG. 31K, the difference being that in the embodiment of FIG. 31K′, the flexible wire 135 is a Bowden cable configured to transfer linear force for directly actuating the flexible portion 187 for expanding and contracting the flexible portion 187 to thereby increase and decrease the diameter of the member 101 to alter the idle tension that the member 101 creates on the tissue wall of the stomach before the medical device is operated for stretching the stomach wall. In the embodiment shown in FIG. 31K′, the member 101 comprises a channel though which the Bowden cable 135 runs, and as such, the Bowden cable 135 stabilizes the member 101.

FIG. 31L shows an embodiment of the member 101 similar to the embodiment shown in FIGS. 31K and 31K′, the difference being that in the embodiment of FIG. 31L, the member is hydraulically operable. The member comprises a hydraulic cylinder 143 placed inside of the flexible portion 187. The hydraulic cylinder 143 is connected to a flexible wire 135 that runs in a channel in the member 101. The expansion and contraction of the flexible portion 187 increases and decreases the diameter of the member 101 to alter the idle tension that the member 101 creates on the tissue wall of the stomach before the medical device is operated for stretching the stomach wall. The hydraulic cylinder 143 is connected to a hydraulic pump placed in a remote unite by means of a fluid conduit 109. Features of the remote unit and the controller are further described with reference to FIGS. 33-65f.

FIG. 31L′ shows an embodiment of the member 101 similar to the embodiment shown in FIG. 31L, the difference being that in the embodiment of FIG. 31L′, the hydraulic cylinder (143) is replaced by an electrical actuator A, which may comprise an electrical motor or a solenoid. The electrical actuator A is connected to a controller by means of an electrical lead 474. The controller may be placed in a remote unit. Features of the remote unit and the controller are further described with reference to FIGS. 33-65FH.

FIGS. 31M and 31M′ shows a medical device 10 for actively stretching a stomach wall of a patient for creating a sensation of satiety. FIG. 31M shows the medical device 10 in its idle state, and FIG. 31M′ shows the medical device 10 in the state in which the operation device has been operated for stretching the stomach wall of the patient. The medical device 10 is implantable and comprises an operable first member 101a configured to be fixated to a first portion of the stomach wall, and a second member 101b configured to be fixated to a second portion of the stomach wall. The medical device 10 further comprises an operation device comprising a flexible wire 135 configured to pull a first portion 190a of the first member 101a in a first direction away from a first portion 190a′ of the second member 101b, for stretching a portion of the stomach wall between the first portion 190a of the first member and the first portion 190a′ of the second member for creating the sensation of satiety.

In the embodiment shown in FIGS. 31M and 31M′, the first and second members 101a, 101b are both curved such that the first and second members 101a, 101b forms circular arcs enclosing portions of the stomach wall. The first member 101a is curved with a curvature having a smaller radius than the curvature of the second member 101b, and the footprint of the first member 101a is smaller than the footprint of the second member 101b.

Also, the upper portion of the first member 101a is placed within the footprint of the second member 101b. The flexible wire 135 is connected to the first member 101a at a first portion 190a of the first member 101a. The first member 101a is made from a flexible material, such as a flexible, elastic, polymer material, such as a silicone-based or polyurethane-based material. The flexible wire is configured to deform the first member 101a from the connection of the flexible wire 135 at the first portion 190a of the first member 101a, as shown in FIG. 31M′. The flexible wire 135 runs through a flexible portion 187 of a sheath, such that the sheath can be compressed as the flexible wire 135 is pulled.

The flexible portion 187 of the sheath is shaped as a bellows with elevated and lowered portions enabling the flexible sheath 187 to be compressed even when fibrotic tissue has grown on the sheath.

As the stomach wall is stretched between the first portion 109a of the first member 101a and a first portion 109a′ of the second member 101b, the stretching takes place in the prolongation of the flexible wire, which means that the stretching occurs mainly in the pulling direction of the flexible wire. This creates an efficient stretching as basically all of the force with which the flexible wire 135 pulls on the first portion 190a of the first member is transferred into stretching force for stretching the stomach wall. I.e. there are no losses related to alteration of the direction of the exerted force.

In the embodiment shown in FIGS. 31M and 31M′, the first member 101a and the second member 101b are connected to each other by means of a connecting portion 188 connecting the outer ends of the first member 101a to the outer ends of the second member 101b. As such, the outer ends of the first member 101a are fixedly fixated to the outer ends of the second member 101b. The flexible wire 135 runs through the connecting portion 188 and the flexible portion 187 is connected to a central portion of the connecting portion 188 and to a central portion of the first member 101a. The connecting portion 188 thus connects the operation device in form of the flexible wire 135 to the second member 101b such that the connection with the second member 101b provides counterforce against the force needed to pull and thereby deform the first portion 190a of the first member 101a in the direction away from the first portion 190a′ of the second member 101b.

FIGS. 31N and 31N′ shows an embodiment of the medical device 10 similar to the embodiment shown in FIGS. 31M and 31M′. The difference being that in the embodiment shown in FIGS. 31N and 31N′, the operation device comprises two flexible wires 135a, 135b. The first flexible wire 135a is connected to a first portion 190a of the first member 101a and the second flexible wire 135b is connected to a second portion 190b of the first member 101a. The embodiment shown in FIGS. 31N and 31N′ further differs in that the circular arcs formed by the curved first and second members 101a, 101b extends further, such that the curvature of the first and second members 101a, 101b almost forms semi-circles. As such, the footprints of the first and second members 101a, 101b encloses larger portions of the stomach wall, and the footprint of the second member 101b almost entirely encloses the first member 101a. The first member 101a is connected to the second member 101b by means of connecting members 188a, 188b, wherein the first connecting member 188a is placed centrally in the first and second members 101a, 101b, in the center of the arcs of the first and second members 101a, 101b, between the first portions 190a, 190a′ and the second portions 190b, 190b′. The connecting portions 188a, 188b thus connects the operation device in form of the flexible wires 135a, 135b to the second member 101b such that the connection with the second member 101b provides counterforce against the force needed to pull and thereby deform the first and second portions 190a, 190b of the first member 101a in the direction away from the first and second portions 190a′, 190b′ of the second member 101b. The flexible members 135a, 135b run on pulleys 144 for altering the direction of the pulling force exerted on the flexible wires 135a, 135b. The pulleys 144 are connected to the second connecting member 188b, and the flexible wires 135a, 135b runs through the second connecting member 188b.

FIG. 31N shows the medical device 10 in its idle state, and FIG. 31N′ shows the medical device 10 in a state in which the operation device in form of the first flexible wire 135a has been operated for moving the second portion 190b of the first member 101a away from the second portion 190b′ of the second member 101b, for stretching the portion of the stomach wall placed between the second portion 190b of the first member 101a and the second portion 190b′ of the second member 101b for creating a sensation of satiety. Just as in the embodiment shown in FIGS. 31M and 31M′, the first member 101a is made from a flexible material, such as a flexible, elastic, polymer material, such as a silicone-based or polyurethane-based material.

FIGS. 31O and 31O′ shows an embodiment of the medical device 10 similar to the embodiment shown in FIGS. 31M and 31M′. The difference being that in the embodiment shown in FIGS. 310 and 310′, the circular arcs formed by the curved first and second members 101a, 101b extends further, such that the curvature of the first and second members 101a, 101b almost forms semi-circles (just as in the embodiment shown in FIGS. 31N and 31N′). As such, the footprints of the first and second members 101a, 101b encloses larger portions of the stomach wall, and the footprint of the second member 101b almost entirely encloses the first member 101a.

Just as in the embodiment shown in FIGS. 31M and 31M′, the operation device comprises a flexible wire 135 configured to pull a first portion 190a of the first member 101a in a first direction away from a first portion 190a′ of the second member 101b, for stretching a portion of the stomach wall between the first portion 190a of the first member and the first portion 190a′ of the second member for creating the sensation of satiety. The first member 101a is made from a flexible material, such as a flexible, elastic, polymer material, such as a silicone-based or polyurethane-based material, and the flexible wire is configured to deform the first member 101a from the connection of the flexible wire 135 at the first portion 190a of the first member 101a, as shown in FIG. 310′. Just as in the embodiment shown in FIGS. 31M and 31M′, the first member 101a and the second member 101b are connected to each other by means of a connecting portion 188 connecting the outer ends of the first member 101a to the outer ends of the second member 101b, such that the outer ends of the first member 101a are fixedly fixated to the outer ends of the second member 101b. The flexible wire 135 runs through the connecting portion 188, and the flexible portion 187 is connected to a central portion of the connecting portion 188 and to a central portion of the first member 101a. The connecting portion 188 thus connects the operation device in form of the flexible wire 135 to the second member 101b such that the connection with the second member 101b provides counterforce against the force needed to pull and thereby deform the first portion 190a of the first member 101a in the direction away from the first portion 190a′ of the second member 101b.

FIG. 31P shows an embodiment of the medical device 10 similar to the embodiment shown in FIGS. 31N and 31N′, the difference being that in the embodiment shown in FIG. 31P the circular arcs formed by the curved first and second members 101a, 101b extends further, such that the curvatures of the first and second members 101a, 101b almost forms full circles or ring-shape. As such, the footprints of the first and second members 101a, 101b encloses even larger portions of the stomach wall, and the footprint of the second member 101b entirely encloses the first member 101a. Just as in the embodiment shown in FIGS. 31N, 31N′ the flexible members 135a, 135b run on pulleys 144 for altering the direction of the pulling force exerted on the flexible wires 135a, 135b. The pulleys 144 are connected to the second connecting member 188b, and the flexible wires 135a, 135b runs through channels in two parts 188b′, 188b″ of second connecting members connecting the first and second members 101a, 101b to each other. The first and second members 101a, 101b further being connected to each other by means of a first connecting member 188a is placed centrally in the first and second members 101a, 101b, in the center of the arcs of the first and second members 101a, 101b, between the first portions 190a, 190a′ and the second portions 190b, 190b′.

The first flexible wire 135a is connected to a first portion 190a of the first member 101a and the second flexible wire 135b is connected to a second portion 190b of the first member 101a, such that pulling on the first flexible wire 135a deforms the first portion 190a of the first member 101a moving the first portion 190a of the first member 101a in a direction away from a first portion 190a′ of the second member 101b, and pulling on the second flexible wire 135b deforms the second portion 190a of the first member 101a moving the second portion 190a of the first member 101a in a direction away from a second portion 190b′ of the second member 101b.

The flexible wires in any of the embodiments described with reference to FIGS. 31I-31P may be operated and controller locally, i.e. close to the medical device, by means of electrical motors operably connected to the flexible wires and a locally placed controller controlling the electrical motors (similar to what is disclosed in the embodiments of FIGS. 31F-31H), or by means of electrical motors and controller placed in a remote unit (similar to what is disclosed in the embodiments of FIGS. 12A—14C and 32-33). Features of the remote unit and the controller are further described with reference to FIGS. 33-65f.

FIG. 31Q schematically shows a medical device 10 for actively stretching a stomach wall of a patient for creating a sensation of satiety. The medical device 10 is implantable and comprises an operable first member 101a configured to be fixated to a first portion of the stomach wall, and a second member 101b configured to be fixated to a second portion of the stomach wall. The medical device 10 further comprises an operation device which for example could comprise a flexible wire 135. The operation device is configured to pull at least a portion of the first member 101a in a first direction away from at least one portion of the second member 101b, for stretching a portion of the stomach wall between the first portion of the stomach wall and the second portion of the stomach wall for creating the sensation of satiety. In the embodiment shown in FIG. 31Q, the medical device 10 is configured to stretch the stomach wall in at least one main stretching direction (indicated by the multiple arrows). The operation device is configured to pull at least a portion of the first member 101a in a pulling direction having an angle relative to the main stretching direction of less than 200 such that the force created by the operation device is translated into a force for stretching the stomach wall with as little loss as possible. Preferably, the operation device is configured to pull at least a portion of the first member 101a in a pulling direction having an angle relative to the main stretching direction of less than 15°, and even more preferably, the operation device is configured to pull at least a portion of the first member 101a in a pulling direction having an angle relative to the main stretching direction of less than 10°.

In the embodiment shown in FIG. 31Q, the first member 101a is fixated to the second member 101b at an articulating joint, such that the first member 101a can pivot in relation to the second member 101b by means of the articulating joint. As such, the most proximal portion of the first member is rotatably fixed to the second member 101b, while the more distal portions of the first member 101a are displaced relative to the second member. As such, most of the first member 101a is moved further away from a first portion 190a′ of the second member 101b, the operation device moving the first portion 190a of the first member 101a closer to a second portion 190b′ of the second member 101b.

In the embodiment shown in FIG. 31Q, the first member 101a is elongated and comprises a first main length axis LA1 in the direction of the elongation, and the second member 101b is elongated and comprises a second main length axis LA2 in the direction of the elongation. The operation device is configured to displace the first member 101a relative to the second member 101b, such that an angle α between the first main length axis LA1 and the second main length axis LA2 is altered.

In the embodiment shown in FIG. 31Q, the operation device comprising a flexible wire 135 is connected to second member 101b at the second portion 190b′, such that the connection with the second member 101b provides counterforce against the force needed to pull the first member 101a in the direction away from the first portion 190a′ of the second member 101b. The structure of the first and second members 101a, 101b may be rigid and may as such be made from a stiff biocompatible material, such as titanium.

FIG. 31Q′ shows an embodiment similar to the embodiment shown in FIG. 31Q, the only difference is that the flexible wire 135 pulling on the first member 101a is replaced by a linear actuator A pushing on the first member 101a. The linear actuator in the embodiment shown in FIG. 31Q′ comprises an electrical motor configured to generate a rotational force. The electrical motor being connected to a threaded “nut” portion configured to engage a threaded screw portion, such that the nut and screw combination transformed the rotational force created by the electrical motor to a linear force for pushing on the first member 101a.

FIG. 31R shows an embodiment of the medical device 10 similar to the embodiment shown in FIG. 31Q, the difference being that in the embodiment shown in FIG. 31R, the first member 101a is not fixated to the second member 101b. In the embodiment shown in FIG. 31R, the first member 101a is parallelly displaced relative to the first portion 190a′ of the second member 101b. Just as in the embodiment shown in FIG. 31Q, the first member 101a is elongated and comprises a first main length axis LA1 in the direction of the elongation, and the second member 101b is elongated and comprises a second main length axis LA2 in the direction of the elongation. The operation device is configured to displace the first member 101a relative to the second member 101b, such that the first main length axis LA1 and the second main length axis LA2 remains parallel. The operation device in the embodiment of FIG. 31R is configured to pull the first member 101a in a pulling direction aligned with the main stretching direction such that the force created by the operation device is translated into a force for stretching the stomach wall with as little loss as possible.

FIG. 31R′ shows an embodiment similar to the embodiment shown in FIG. 31R, the difference is that the flexible wire 135 pulling on the first member 101a is replaced by a linear actuator A pushing on the first member 101a. The linear actuator in the embodiment shown in FIG. 31R′ comprises an electrical motor configured to generate a rotational force. The electrical motor being connected to a threaded “nut” portion configured to engage a threaded screw portion, such that the nut and screw combination transformed the rotational force created by the electrical motor to a linear force for pushing on the first member 101a.

FIGS. 31S and 31T shows an embodiment of the medical device 10 similar to the embodiment shown in fog. 31R. The difference is that in the embodiment shown in FIGS. 31S and 31T, the operation device comprises two flexible wires 135a, 135b, each being connected to a first and second portion 190a, 190b of the first member 101a. Just as in the embodiments shown in FIGS. 21Q and 31R, the second members are substantially U-shaped and the flexible members 135a, 135b are connected to the second member 101b at a second and third portion 190b′, 190c′. When the first flexible wire 135a is pulled, the first portion 190a of the first member 101a is moved closer to the second portion 190b′ of the second member 101b, such that the first portion 190a of the first member 101a is moved further away from the first portion 190a′ of the second member 101b which stretches the stomach wall between the first portion 190a of the first member 101a and the first portion 190a′ of the second member 101b. Just as in the embodiment shown in FIG. 31Q, displacement of the first portion of the first member 101a relative to the second member 101b creates an angle α, or increases the angle α, between the first main length axis LA1 and the second main length axis LA2. When the second flexible wire 135b is pulled, the second portion 190b of the first member 101a is moved closer to the third portion 190c′ of the second member 101b, such that the second portion 190b of the first member 101a is moved further away from a fourth portion 190a″ of the second member 101b, which stretches the stomach wall between the second portion 190b of the first member 101a and the fourth portion 190a″ of the second member 101b. This also creates an angle α, or increases the angle α, between the first main length axis LA1 and the second main length axis LA2. With the embodiment of FIGS. 31S and 31T, to different areas of the stomach wall can be selected for stretching.

FIGS. 31S′ and 31T′ show embodiments similar to the embodiments shown in FIGS. 31S and 31T, the difference is that the flexible wire 135 pulling on the first member 101a is replaced by linear actuators A1, A2 pushing on the first member 101a. The linear actuators A1, A2 in the embodiment shown in FIGS. 31S′ and 31T′ comprises electrical motors configured to generate rotational force. The electrical motors being connected to threaded “nut” portions configured to engage threaded screw portions, such that the nut and screw combination transformed the rotational force created by the electrical motor to a linear force for pushing on the first member 101a.

FIGS. 11-14D′, 28A-31I, 31M-31T are examples of embodiments in which the medical device comprises an operable first member configured to be fixated to a first portion of the stomach wall, a second member configured to be fixated to a second portion of the stomach wall, and an operation device. Generally, a non-therapeutic method of creating a sensation of satiety using a pre-implanted medical device configured to stretch a portion of the stomach wall of a human is further provided. The non-therapeutic method generally comprises: pulling at least a portion of the first member in a first direction away from at least one portion of the second member, for stretching a portion of the stomach wall between the first portion of the stomach wall and the second portion of the stomach wall for creating the sensation of satiety.

The non-therapeutic method of creating a feeling of satiety could be linked to a life-style choice of not eating too much or could be linked to a desired physical appearance and as such be linked to physical esthetics.

The medical devices of FIGS. 11-14D′, 28A-31I, 31M-31T may comprise or be connected to an implantable controller configured to control the operation of the first member. In such embodiments, the non-therapeutic method comprises operating the first member as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

FIGS. 31U-31U″ shows an embodiment of a medical device 10 for stretching the stomach wall of a patient. The medical device 10 shown in FIGS. 31U-31U″ comprises two first members 101a′, 101a″ and two second members 101b′, 101b″. The left first member 101a′ and the left second member 101b′ forms a first stretching unit and the right first member 101a″ and the right second member 101b″ forms a second stretching unit. The first and second stretching units operates in the same way but are configured to stretch different portions of the stomach wall and by alternatingly operating the first and second stretching unit, different areas of the stomach wall can be stretched, which means that the portion of the stomach wall not being stretched can recover. Allowing the stretched tissue to recover is important as fatigue in the tissue may have a negative effect on the tissues ability to generate sensations of satiety, i.e. an over-stretched stomach portion may become immune to the stretching resulting in that the function of the creation of an artificial sensation of satiety through stretching disappears.

The first stretching unit comprises a first member 101a′ comprising a first portion 190a′, a second portion 190b′ and a third portion 10c′. The three portions 190a′-190c′ forms a closed curve together with a common portion 190 which connects one end portion of the first portion 190a′ with one end portion of the second portion 190c′. End portions of the four portions (the first—third portion 190a′-190c and the common portion 190) are connected by means of articulating joints or points of pivot 131a-131d, such that the portions can pivot in relation to each other. More specifically, in the first stretching unit, the first portion 190a′ is connected to the common portion 190 by means of a first point of pivot 131a, the first portion 190a′ is connected to the second portion 190b′ by means of a second point of pivot 131b′, the second portion 190b′ is connected to the third portion 190c′ by means of a third point of pivot 131c′, and the third portion 190c′ is connected to the common portion 190 by means of a fourth point of pivot 131d. The portions 190a″-190c″ of the second stretching unit are connected to each other in the same way.

The third portion 190c′ is connected to an operation device, which in the embodiment shown in FIG. 31U is a hydraulic cylinder 143a which is operated by means of pressurized hydraulic fluid conducted to the hydraulic cylinder 143a by means of a conductor 109a, fluidly connecting the hydraulic cylinder 143a to a source of pressurized fluid, such as a hydraulic pump placed in a remote unit. Extension and compression of the hydraulic cylinder 143a causes the third portion 190c′ to pivot relative to the common portion 190. A second hydraulic cylinder 143b operates the third portion 190c″ of the second stretching unit in the same way.

The first stretching units each further comprises a left and right second member 101b′, 101b″, which are separate from, but positioned within the footprint, of the left and right first members 101a′, 101a″, respectively.

FIG. 31U′ shows the medical device 10 in a state in which the first hydraulic cylinder 143a has been extended and thereby has operated the third portion 190c′ of the first stretching unit and has caused the third portion 190c′ to pivot upwards relative to the common portion 190. This pivot propagates in the first stretching unit such that the third portion 190c′ pivots relative to the second portion 190b′, the second portion 190b′ pivots relative to the first portion 190a′, and the first portion 190a′ pivots relative to the common portion 190 such that the shape of the closed curve formed by the portions 190-190c′ of the first stretching unit alters from being substantially a square to being substantially a rhombus. The alteration of the shape of the first stretching unit moves the first point of pivot 131a closer to the third point of pivot 131c′ and thereby the first point of pivot 131a closer to a first portion 190a″′ of the second member 101b′ and the third point of pivot 131c′ closer to a third portion 190c″′ of the second member 101b′. Simultaneously, the second point of pivot 131b′ is moved further away from the fourth point of pivot 131d and thereby the second point of pivot 131b′ is moved further away from a second portion 190b″′ of the second member 101b′ and the fourth point of pivot 131d is moved further away from a fourth portion 190d″′ of the second member 101b′. The increase in distance between the second point of pivot 131b′ and the second portion 190b″′ of the second member 101b′ stretches the stomach wall placed between the second point of pivot 131b′ and the second portion 190b″′ of the second member 101b′, and the increase in distance between the fourth point of pivot 131d and the fourth portion 190d″′ of the second member 101b′ stretches the stomach wall placed between the fourth point of pivot 131d and the fourth portion 190d″′ of the second member 101b′.

FIG. 31U″ shows the medical device 10 in a state in which the second hydraulic cylinder 143b has been compressed and thereby operated the third portion 190c″ of the second stretching unit and has caused the third portion 190c″ to pivot downwards relative to the common portion 190. This pivot propagates in the second stretching unit such that the third portion 190c″ pivots relative to the second portion 190b″, the second portion 190b″ pivots relative to the first portion 190a″, and the first portion 190a″ pivots relative to the common portion 190 such that the shape of the closed curve formed by the portions 190-190c″ of the second stretching unit alters from being substantially a square to being substantially a rhombus. The alteration of the shape of the second stretching unit moves the second point of pivot 131b″ closer to the fourth point of pivot 131d and thereby the second point of pivot 131b″ closer to a second portion 190b″″ of the second member 101b′ and the fourth point of pivot 131d closer to a fourth portion 190d″″ of the second member 101b′. Simultaneously, the first point of pivot 131a is moved further away from the third point of pivot 131c″ and thereby the first point of pivot 131a is moved further away from a second portion 190b″″ of the second member 101b′ and the third point of pivot 131c″ is moved further away from a third portion 190c″″ of the second member 101b′. The increase in distance between the first point of pivot 131a and the second portion 190b″″ of the second member 101b′ stretches the stomach wall placed between the first point of pivot 131a and the second portion 190b″″ of the second member 101b′, and the increase in distance between the third point of pivot 131c″ and the third portion 190c″″ of the second member 101b′ stretches the stomach wall placed between the third point of pivot 131c″ and the third portion 190c″″ of the second member 101b′.

FIGS. 31V and 31V′ shows an embodiment of the medical device 10 configured to stretch the stomach wall between a first member 101a and a second member 101b. The first member 101a being a ring shaped first member 101a forming a closed curve and being connected to, and placed within, the footprint of a ring-shaped second member 101b. The first and second members 101a, 101b both being configured to be fixated to the stomach wall by means of the first and second members 101a, 101b being at least partially invaginated in the stomach wall such that tissue of the stomach all is folded over the first and second members, respectively, whereby the first and second members are fixated in tissue tunnels created by the tissue wall of the stomach.

In the embodiment shown in FIGS. 31V and 31V′, the first member 101a comprises a first member part 101a′ which is hollow and made up of a plurality of flexible portions 187a, 187b, 187c connected to each other for forming the ring shaped first member part 101a′. The flexible portions 187a, 187b, 187c are preferably made from an elastic polymer material such that the first member part 101a′ can elastically deform. The hollow channel enclosed by the plurality of flexible portions 187a, 187b, 187c is configured to house a second member part 101a″ in the form of a semi-circular arc comprising an outer threaded portion 132 configured to engage and be propelled by a gear wheel 137 connected to the second member 101b. As the second member part 101a″ is semi-circular, it (relative to a full circle) forms a rigid section and an open or flexible section, i.e., the open section being the section of the hollow first member part 101a′ void of the semi-circular second member part 101a″. As the second member part 101a″ is displaceable, i.e., rotatable, relative to the first member part 101a′, the position of the open or flexible section of the second member part 101a″ can be altered. As such, the position of a flexible first portion 190a configured to be operated to deform, for changing the stomach wall portion to be stretched between the flexible first portion 190a of the first member 101a and the first portion 190a′ of the second member 101b.

The plurality of flexible portions 187a, 187b, 187c forms elevated and lowered portions on the first member part 101a″, such that the first member part 101a″ becomes bellows shaped, such that the first member part 101a″ can remain flexible even as fibrotic tissue overgrows the first member part 101a″. In the embodiment shown in FIGS. 31V and 31V′, a major portion of the second member 101b is made from a material having a modulus of elasticity in the range 50 GPa-400 GPa.

The medical device shown in FIGS. 31V and 31V′ further comprises a flexible wire 135 configured to be housed within the first member part 101a and configured to be wound on a drum 146 or spool such that the portion of the flexible wire 135 housed within the channel enclosed by the plurality of flexible portions 187a, 187b, 187c can be made longer and shorter by the rotation of the drum 146.

FIG. 31V′ shows the medical device 10 in a state in which the second member part 101a″ has been rotated such that a first portion 190a has a center placed between end portions of the second member part 101a″ coinciding with a first portion 190a′ of the second member in a clock direction being substantially 10.30. The flexible wire has been wound on the spool 146 such that the portion of the flexible wire 135 housed within the channel enclosed by the plurality of flexible portions 187a, 187b, 187c has been made shorter causing the first portion 190a to be moved closer to the center of the first member 101a and thereby further away from the first portion 190a′ of the rigid second member 101b such that the stomach wall between the first portion 190a of the first member 101a and the first portion 190a′ of the second member 101b is stretched.

The drum 146 and the gear wheel 137 can be powered by electric motors placed locally, in direct connection with the medical device 10. In the alternative, the drum 146 and/or the gear wheel 137 may be powered by and electric motor placed in a remote unit, in which case a rotating flexible shaft may be used to transfer mechanical force to the medical device (such as for example shown in FIG. 7). In the alternative, the drum 146 and the gear wheel 137 may be powered by a hydraulic motor fluidly connected to a source of pressurized fluid, such as a hydraulic pump placed in a remote unit. The medical device 10 may further comprise a controller configured to control the operation of the drum 146 and the gear wheel 137 independently of each other. Just as the motors, the controller may be placed locally, in connection with the medical device 10, or in a remote unit. Features of the remote unit and the controller are further described with reference to FIGS. 33-65f.

FIG. 31X shows an embodiment of the medical device 10 comprising an operable first member 101a forming a closed curve having a hexagonal shape encircling a portion of the stomach wall and being configured to be fixated to the stomach wall by means of the operable first member being invaginated by a portion of the stomach wall. The operable first member 101a is configured to be displaced relative to the second member 101b such that a portion of the stomach wall between the first and second members 101a, 101b is stretched by the displacement. The second member 101b forms a simple closed curve by being circular or substantially ring or torus shaped, encircling a portion of the stomach wall. The second member 101b is also configured to be fixated to the stomach wall by means of invagination. The medical device 10 according to the embodiment shown in FIG. 31X comprises an operation device configured to move the first member in a first, second and third direction relative to the second member, for stretching portions of the stomach wall between the first, second and third members.

The medical device 10 further comprises a mechanical switching device 149a, 149b, 149c configured to switch the operation device from operating the first member 101a to move in a first direction to operating the first member 101a to move in a second direction and from operating the first member 101a to move in a second direction to operating the first member 101a to move in a third direction.

The first member 101a have a largest cross-sectional distance being smaller than the largest cross-sectional distance of the second member 101b. In alternative embodiments, the first member is circular, i.e. ring-shaped, and have a first radius, which may be smaller than a second radius being the radius of the second member 101b.

In the embodiment shown in FIGS. 31X-31X″′, the first member encloses a smaller portion of the stomach wall than the second member 101b, and the footprint of the first member 101a is smaller than the footprint of the second member 101b.

The first and second members 101a, 101b together forms a main portion, being the portion placed in close proximity to the stomach wall. The second member 101b is substantially rigid and a major portion of the second member 101b is made from a material having a modulus of elasticity in the range 50 GPa-400 GPa, such as titanium.

The operation device comprises a propulsion device, in the form of a flexible shaft 135.

The flexible shaft transfers force from a remote unit (not shown) and is configured to transfer both linear and rotational movement. The flexible shaft 135 travels from the remote unit to the second member enclosed in a sheath or protective cover 136 until it enters the second member (at a receiving portion), in which it travels in a channel 191. The flexible shaft 135 comprises a first, second and third engaging portion 139a, 139b, 139c in the form of a star-shaped spline shaft configured to engage a first, second and third operating element 146a, 146b, 146c in the form of a first, second and third drum rotatably attached in the second member 101b. The first, second and third engaging portion 139a, 139b, 139c are distributed along the flexible shaft and spaced apart such that only one of the engaging portions engages an operating element, whereas two of the engaging portions does not engage an operating element. In the state shown in FIG. 31X, the first engaging portion 139a engages the first operating element (drum) 146a, such that the flexible shaft propels the drum 146a by means of the engagement between the first engaging portion 139a and the first drum 146a. When the flexible shaft 135 is moved linearly, in its axial direction, the positions of the engaging portions 139a-139c are moved relative to the operating elements 146a-146c. When the flexible shaft 135 is moved such that the positions of the engaging portions 139a-139c are moved one step, the first engaging portion 139a disengages from the first operating element, simultaneously the second engaging portion 139b engages the second operating element 146b, such that the flexible shaft 135 propels the second drum 146b by means of the engagement between the second engaging portion 139b and the second drum 146b, this is the state illustrated in FIG. 31X″′. When the flexible shaft 135 is moved further such that the positions of the engaging portions 139a-139c are moved one more step (not shown), the second engaging portion 139b disengages from the second operating element 146b, simultaneously the third engaging portion 139c engages the third operating element 146c, such that the flexible shaft 135 propels the third drum 146c by means of the engagement between the third engaging portion 139c and the third drum 146c.

In the first state, shown in FIG. 31X, the flexible shaft 135 propels the first drum 146a, which is placed at a first portion 190b′ of the second member 101b, for winding and unwinding a flexible wire 135a on the first drum 146a. This is shown in more detail in FIG. 31X′. When the first drum 146a is propelled in a first direction, the flexible wire 135a is wound onto the first drum 146a. The flexible wire 135a is protected by a flexible sheath 187 comprising elevated and lowered portions, such that the sheath can remain flexible even as fibrotic tissue overgrows the sheath. The flexible wire is fixedly fixated to a first portion 190a of the first member 101a. When the flexible wire 135a is wound onto the first drum 146a, the first member 101a is pulled towards the first portion 190a′ of the second member 101b, which decreases the distance between the first portion 190a of the first member 101a and the first portion 190a′ of the second member 101b and increases the distance between a fourth portion 190d of the first member 101a and a fourth portion 190d′ of the second member 101b, which stretches the stomach wall placed between the fourth portion 190d of the first member 101a and the fourth portion 190d′ of the second member 101b.

FIG. 31X′ shows a cross-section A-A of the second portion of the switching device 149a in a more detailed view. In the upper version of the cross-section A-A, the first engaging portion 139a comprises a single protrusion configured to engage a single protrusion of the first drum 146a. As the single protrusions makes up only a small portion of the space between the first drum 146a and the engaging portion 139a, the risk that the single protrusion of the engaging portion 139a will engage the single protrusion of the first drum 146a and disturb the insertion of the engaging portion in the drum is very small. In the lower version of the cross-section A-A, the engaging portion is star-shaped i.e. comprises elements similar to splines. The star-shaped embodiment provides more guidance and further engagement surfaces, which means that the first drum 146a will rotate along with the engaging portion 139a immediately when the engaging portion 139a starts to rotate. This can be an advantage when control of the rotation of the first drum 146a by the engagement of the engaging portion 139a is important such as when the exact location of the first member 101a relative to the second member 101b is important.

FIG. 31X″ shows a cross-section B-B of the second portion of the switching device 149b in a more detailed view. In FIG. 31X″ it is shown that the second engaging portion 139b does not engage the second operating element 146b. It can further be seen how the flexible wire 135b is wound on the second drum 146b.

FIG. 31X″′ shows the medical device 10 in the second state, in which the flexible shaft 135 has been moved such that the second engaging portion 139b propels the second drum 146b, which is placed at a second portion 190b′ of the second member 101b, for winding and unwinding a flexible wire 135b on the second drum 146a. In the state shown in FIG. 31X″′ the second drum has been propelled such that the flexible wire 135b is wound onto the second drum 146b and first member 101a has been pulled towards the second portion 190b′ of the second member 101b, which has decreased the distance between the second portion 190b of the first member 101a and the second portion 190b′ of the second member 101b and increased the distance between a fifth portion 190e of the first member 101a and a fifth portion 190e′ of the second member 101b, which stretches the stomach wall placed between the fifth portion 190e of the first member 101a and the fifth portion 190e′ of the second member 101b.

In the embodiment shown in FIGS. 31X-31X″′ the flexible wires are directly connected to the first member 101a at the first, second and third portions. However, in an alternative embodiment, the flexible wires may be connected to the first member via pulleys for creating transmission, such as for example shown in FIGS. 31D, 31E and 31H.

In the embodiment shown in FIGS. 31X-31X″′ the first member 101a can be displaced in a first, second and third direction relative to the second member 101b. The angle between the first, second and third directions is substantially 120°.

In the embodiment shown in FIGS. 31X-31X″′ the operation device and the switch is partially placed in a remote unit. More specifically, in the embodiment shown in FIGS. 31X-31X″′, an electrical motor, gear box, energy storage unit and controller is placed in the remote unit. Features of the remote unit and the controller are further described with reference to FIGS. 33-65f.

In the embodiment shown in FIGS. 31X-31X″′ the operation device is configured to move the first member 101a relative to the second member 101b by displacing the first member 101a relative to the second member, however, in alternative embodiments, the operation device may be configured move the first member 101a relative to the second member 101b by deforming at least one flexible portion of the first member. Such flexible portion may comprise at least one elastic portion which may comprise at least one elastic material. Examples and further description of deforming first members are further provided in FIGS. 31E and 31M-31P.

The first and/or second members shown in FIGS. 31X-31X″′ may be modified such that at least one of the length and the cross-sectional area of at least one of the first and second member is adjustable for calibrating the stretching of the stomach wall. Examples of this feature, which may be implemented in the first and/or second members 101a, 101b in FIGS. 31X-31X″′, are shown and described in FIGS. 31J-31L′.

FIG. 31Y shows an alternative implementation of the concept described with reference to FIGS. 31X-31X″″. In FIG. 31Y, the concept has been implemented in an embodiment of the medical device 10 similar to that described with reference to FIG. 7. The difference being that the point of pivot 131 of the third member 101c has been displaced to the right and the point of pivot 131 of the fourth member 101d has been displaced to the left. When the worm 133 is in its first, proximal position, it engages the worm wheel 132 of the third member 101c for operating the third member, and when the worm is in its second, distal position, it engages the worm wheel 132 of the fourth member 101d, for operating the fourth member 101d. The worm wheel 133 switches between the first and second position by means of the rotatable shaft 134 connected to the flexible shaft 135 is extended and retracted.

FIGS. 31Y′-31Y″″ shows different embodiments of a remote unit 140. In the embodiment shown in FIG. 31Y′, the remote unit 140 comprises a switch 183, 184a, 184b, operable by centrifugal force. An electrical motor MO is operably connected to a gear system G which reduces the speed of the electrical motor MO. The gear system G is in turn connected to a first portion of the switch operable by centrifugal force. The first portion comprises a force output element comprising two pivotable arms 183 which are connected to each other with a spring 145 acting to pull the two pivotable arms 183 towards each other. As the pivotable arms 183 rotates along with the electrical motor M, the pivotable arms are pressed outwards by the centrifugal force. At low speed, the force exerted by the spring 145 is stronger than the force exerted by the centrifugal force, which forces the arms 183 towards each other and in engagement with a force receiving element in form of a first engagement member 184a, causing the telescopic shaft portion 178 and thereby the flexible shaft 135 to rotate. When the speed is increased, the arms 183 are forced apart, causing the arms to engage the second force receiving element in the form of a second engagement member 184b causing relative rotation between the second engagement member 184b and the shaft 134. The shaft 134 comprises a threaded portion 481t and the second engagement member 184b comprises a nut portion 177 comprising inner threads. The relative rotation between the second engagement member 184b and the shaft 134 causes an axial displacement of the shaft 134 and thereby of the flexible shaft 135. The axial movement of the flexible shaft 135 propagates to the worm 133 and causes the worm 133 to switch from operating the worm wheel 132 connected to the third member, to operating the worm wheel 132 connected to the fourth member 101d.

In the embodiment shown in FIG. 31Y″, the switch comprises a second electrical motor MO2, configured to only generate axial movement by the connection with a transmission comprising a threaded portion 481t of the shaft and a not portion 177 connected to the second electrical motor MO2.

The transmission thus transforms the rotational movement generated by the electrical motor MO2 to a linear, axial movement causing an axial displacement first electrical motor MO1, the gear system G, and the flexible shaft 135.

FIG. 31Y″′ shows an embodiment similar to that described with reference to FIG. 31Y′, the difference being that in the embodiment shown in FIG. 31Y″′, the first engagement member 184a comprises a sleeve, just as the second engagement member 184b. The first engagement member 184a is connected to the telescopic shaft portion 178, just as in the embodiment shown in FIG. 31Y′. At a first speed, the spring 145 keeps the arms 183 connected to each other, such that the switch is in a neutral position in which the rotation of the arms 183 does not move the flexible shaft 135 in any direction. When the speed is increased, the arms 183 are forced apart, causing the arms to engage the first engagement member 184a causing rotation of the flexible shaft 135. When the speed is further increased, the arms 183 passes the engagement portion of the first engagement member 184a and continues outwards, such that the arms engage and propels the second engagement member 184b, causing relative rotation between the second engagement member 184b and the shaft 134. Just as in the embodiment of FIG. 31Y′, the relative rotation between the second engagement member 184b and the shaft 134 causes an axial displacement of the shaft 134 and thereby of the flexible shaft 135. The axial displacement of the flexible shaft 135 propagates to the worm 133 and causes the worm 133 to switch from operating the worm wheel 132 connected to the third member, to operating the worm wheel 132 connected to the fourth member 101d.

FIG. 31Y″″ shows an embodiment similar to that described with reference to FIG. 31Y″′, the difference being that in the embodiment shown in FIG. 31Y″″, the first engagement member 184a comprises the nut portion 177 comprising inner threads configured to engage a threaded portion 481t of the shaft 134, such that rotation of the first engagement member 184a creates axial displacement of the flexible shaft 135 which propagates to the worm 133 and causes the worm 133 to switch from operating the worm wheel 132 connected to the third member, to operating the worm wheel 132 connected to the fourth member 101d. The second engagement member 184b is connected to the telescopic shaft portion 178 such that rotation of the second engagement member 184b creates rotation of the flexible shaft 135.

The remote units 140 shown in FIGS. 31Y′-31Y″″ are described in relation to the medical device 10 of FIG. 31Y, however, the remote units 140 in 31Y′-31Y″″ are equally suitable for operating the medical device of FIGS. 31X-31X″′.

In a broader sense, the remote units 140 shown in FIGS. 31Y′-31Y″″ are suitable for operating all types of implants having an active, or body engaging portion, configured to receive both linear and rotational force and should be seen as a stand-alone element which may be implemented in a wide range of implants.

FIGS. 31Y-31Y″″ are examples of embodiments in which the implanted medical device comprises: an operable first member configured to be fixated to a first portion of the stomach wall, a second member configured to be fixated to a second portion of the stomach wall, an operable third member configured to be fixated to a third portion of the stomach wall, an operation device, and a mechanical switching device connected to the operation device. Generally, a non-therapeutic method of creating a sensation of satiety using a pre-implanted medical device configured to stretch a portion of the stomach wall of a human is further provided. The non-therapeutic method generally comprises operating the mechanical switching device such that the mechanical switching device switches the operation device from operating the first member to move in a first direction to operating the third member to move in a third direction.

The non-therapeutic method of creating a feeling of satiety could be linked to a life-style choice of not eating too much or could be linked to a desired physical appearance and as such be linked to physical esthetics.

The medical devices of FIGS. 31Y-31Y″″ may comprise or be connected to an implantable controller configured to control the operation of the mechanical switching device. In such embodiments, the non-therapeutic method comprises operating the mechanical switching device as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

The members (101a-101d) of the embodiments shown in FIGS. 28A-31Y are fixated to the stomach wall by means of invagination, meaning that tissue form the stomach wall is folded over the structure of the respective member and the tissue is sutured or stapled stomach-to-stomach, such that the tissue is connected by means of fibrotic tissue generation i.e. the tissue grows together over the member this creating a stable long-term fixation which after some period no longer relies on the fixation created by the sutures or staplers. The invagination may be performed on portions of the first and/or second member, or may be performed on the entire first and/or second member. In some embodiments, the medical device is configured to be completely invaginated by the stomach wall apart from elements for transferring force or communication to or from the medical device. Complete invagination improves the long-term fixation and reduces the risk that the function of the device is adversely affected by unexpected ingrowth of fibrotic tissue or the connection with other organs in the body. Complete invagination also reduces the risk that other organs in the body of the patient is inadvertently affected by the medical device, reducing the risk of e.g. pain, damage to the thoracic diaphragm, damage to the liver, damage to the intestines (such as ileus), damage to the esophagus or damage to the spleen etc..

The members of the embodiments shown in FIGS. 28A-34H are described as being operated by mechanical and/or hydraulic operation devices. However, a mechanical or electrical operation device in any of the embodiments shown in FIGS. 28A-34H may be replaced by any of the other hydraulic or mechanic operation devices shown herein. Electrical motor(s) transforming electrical energy to mechanical force may be placed locally in vicinity to the members of the medical device or may be placed in a remote unit. The electrical motor(s) may be configured to act on the members by a mechanical connection, such as a flexible wire (or Bowden cable), a rotating shaft or a gear or worm wheel connection. In the alternative, electrical motor(s) may be configured to power a hydraulic pump in fluid connection with a hydraulic operation device (such as a hydraulic cylinder or hydraulic motor) in turn operating the members for stretching the tissue of the stomach wall.

FIG. 32A shows the stomach S of a patient when two medical devices 10a, 10b according to the embodiment described with reference to FIGS. 28a-28c have been fixated to the stomach wall SW of the fundus F for actively stretching the stomach wall SW of the fundus F for inducing a sensation of satiety. Having two medical devices 10a,10b for actively stretching the stomach wall SW of the patient enables the alternation of stomach wall SW portions for stretching, which reduces the risk that the satiety inducing effect is reduced by fatigue of the stretch receptors in the stomach wall SW. The first members 101a of the first and second medical devices 10a, 10b are fixated to the stomach wall SW of the fundus F by means of invagination using stomach-to-stomach sutures or staplers. Each first member 101a is operated by means of flexible wires 135 traveling inside of protective covers 136a,136b, which combine to a single protective cover for guiding the flexible wires 135 to a remote unit 140 for remote operation of the first members 101a.

The remote unit 140 of the embodiment shown in FIG. 32A is configured to pull on the flexible wires 135 in two directions by means of the flexible wires 135 being looped around pulleys 144d′ 144c′, wherein one pulley 144c′ is configured for operating the first member 101a of the first medical device 10a and another pulley 144d′ is configured for operating the first member 101a of the second medical device 10b. The flexible wire is in the embodiment of FIG. 32A a metal wire made from a plurality of strands, but could however also be a wire made from a flexible polymer material. The remote unit 140 comprises a first motor MO1 which is connected via a belt drive to the pulley 144d′ and a second motor MO2 is connected by means of a belt drive to the pulley 144c′. The motors MO1, MO2 are suspended and pulled back by a spring 145 which creates pre-tension in the cables 135 which eliminates slack end ensures that the cables 135 remain threaded onto the pulleys 144d′, 144c′ at all time.

The motors MO1 and MO2 are in the embodiment shown in FIG. 32A electrical motors with integrated gearboxes to lower the speed of the electrical motors to an RPM suitable for operating the worm drive. The motor could for example be an implantable brushless DC motor with integrated gear box, such as the motors provided by Maxon group or Dr. Fritz Faulhaber. The gearboxes integrated with the electrical motors could in alternative embodiments be supplemented or replaced by gearing provided by block and tackle functionality. Such functionality is further described with reference to FIGS. 12A—12E and could be placed in the remote unit 140 or in direct connection with the two medical devices 10a, 10b.

In the embodiment of FIG. 32A, the remote unit comprises a first and second portion 141′,141″ placed on different sides of a portion of muscle tissue MT of the patient, and connected by means of a connecting portion placed through a hole in the muscle tissue MT. The second portion 141′ is placed on the inside of the muscle tissue MT and the first portion is placed on the outside of the muscle tissue MT in the subcutaneous tissue ST. In the embodiment shown in FIG. 32A, the controller 300 is placed in the second portion 141″, and the implantable energy storage unit 40 is placed in the first portion 141′. The controller 300 and the implantable energy storage unit 40 are electrically connected by means of a lead running in the connecting portion, such that electrical energy and communication can be transferred from the second 141″ to the first portion 141′, and vice versa. In the embodiment of FIG. 32A, the first portion 141′ further comprises a wireless energy receiver 305 for receiving wireless energy for charging the implantable energy storage unit 40 and/or for powering the medical device, and a transceiver 308 for receiving and/or transmitting wireless signals to/from the outside the body. The implantable energy storage unit 40 may be any type of energy storage unit suitable for an implant, such as a re-chargeable battery or a solid-state battery, such as a tionyl-chlorid battery. The energy storage unit may be equipped with an energy storage unit indicator configured to indicate a functional status of the implantable energy storage unit. The functional status may indicate at least one of charge level and temperature of the implantable energy storage unit 40. For enabling indication of the temperature of the energy storage unit 40, the energy storage unit 40 or medical device 10 may comprising a temperature sensor.

The controller 300 may comprise at least one sensor, or be configured to receive sensor input from the at least one sensor. The sensor could be a sensor configured to sense a physical parameter of the medical device system, such as at least one of:

A temperature of the medical device system, to avoid excessive heating of tissue connected to the medical device during operation of the medical device or charging of the energy storage unit 40. Excessive heating may also damage the medical device and/or the energy storage unit 40. Excessive heating may also be an indicator that something is wrong with the medical device 10 and may be used for triggering an alarm function for alerting the patient or physician.

A parameter related to the power consumption of the medical device system, to avoid excessive power consumption which may drain and/or damage the energy storage unit 40. Excessive power consumption may also be an indicator that something is wrong with the medical device 10 and may be used for triggering an alarm function for alerting the patient or physician.

A parameter related to strain in the medical device, such as the strain in the motors MO1, MO2, the flexible wires 135 or in the first member 101a (and in the second member in alternative embodiments). Strain can be measured to avoid excessive strain which may damage medical device or the tissue of the patient. Excessive strain may also be an indicator that something is wrong with the medical device 10 and may be used for triggering an alarm function for alerting the patient or physician.

A parameter related to the wireless transfer of energy from a source external to the body of the patient. Excessive transfer of wireless energy may damage an implanted wireless energy received or the energy storage unit. It may also create excessive heating which may damage the tissue of the patient.

The controller 300 may comprise a sensor or be configured to receive sensor input from a sensor configured to sense a physiological parameter of the patient. The physiological parameter of the patient may be:

A parameter related to the patient swallowing, such that the medical device can be controlled on the basis of the patient eating or drinking. A sensor configured to sense a parameter related to the patient swallowing could comprises a motility sensor, which could be a piezo electric or piezo resistive motility sensor, or an accelerometer. In the alternative, a acoustic sensor, such as a microphone, may be used to sense the patient swallowing by picking up the sound generated by the patient swallowing. In the alternative, an optical sensor may be used for sensing the opacity alteration over the esophagus as food passes. A strain sensor could also be used for sensing the expansion of the esophagus as food passes. The features and functions of sensors sensing the patient swallowing is further described with reference to FIG. 16.

A local temperature, to avoid local excessive heating which may damage tissue of the patient.

A systemic temperature, to avoid systemic excessive heating which may cause fever and affect the overall wellbeing of the patient.

Blood saturation/oxygenation, or a parameter related to an ischemia marker such as lactate, to control and/or avoid that the flow of blood to some tissue portion is hampered by the implantation or operation of the medical device 10. Hampered blood flow may lead to tissue damage and in the worst cases to tissue necrosis.

Blood pressure, which may be an indication that the strain created by the medical device is in some way damaging to the overall wellbeing of the patient. Increased blood pressure may be used for triggering an alarm function for alerting the patient or physician.

pH, for determining the acidity of the stomach, which could be an indicator of the function of the digestive system and/or of the frequency of ingestion. The pH may be used for controlling the medical device 10 on the basis of the patient eating or drinking.

In some embodiments, the medical device further comprises a capacitor connected to the implantable energy storage unit 40 and connected to the motors MO1, MO2. The capacitor is configured to be charged by the implantable energy storage unit 40 and to provide the operation device with electrical power. The capacitor can compensate for situations in which the discharging of energy from the implantable energy storage unit 40 during startup of the motors MO1, MO2 is slower than the energy needed for startup of the motors. That is, there is a difference between the energy needed by the operation device and the energy the implantable energy storage unit 40 is capable of providing without damaging the implantable energy storage unit 40.

The controller 300 may further comprise a receiver for receiving patient generated control signals from a unit located external to the body of the patient. The receiver could be a wireless receiver configured to communicate with a transmitter located external to the body of the patient. The controller 300 may be configured to control the operation device 100 on the basis of the received patient generated control signal. The control signal could for example being that the patient indicates to the medical device 10 that the patient has finished a portion of food which causes the medical device 10 to operate to stretch the stomach wall SW of the patient such that satiety is created which reduces the patients willing to eat further. In the alternative, the controller 300 may be configured to control the operation device 100 on the basis of a signal related to a lapsed time or a time of day such that satiety can be created with certain intervals or during specific periods of the day. The controller 300 could further be configured to receive a signal from a sensor external to the body of the patient and use such signal for controlling the operation of the medical device 10. The sensor external to the body of the patient could be a sensor could be a sensor measuring a parameter related to the patient eating to create input for the control of the medical device 10. Such a parameter could be related to body temperature, blood pressure or the glucose level of the blood. In the alternative, the sensor could be a sensor sensing a parameter related to the external environment, such as the atmospheric pressure, which could affect the pressures in the medical device 10.

In alternative embodiments in which the medical device is hydraulically operated (such as described with reference to FIGS. 29a-29c), the remote unit is a hydraulic remote unit, such as for example disclosed in FIGS. 15 or 35A-35B configured to transport hydraulic force to hydraulic actuator(s) by means of implantable conduits, e.g. for operating the first member of the medical device of FIGS. 29A—29C.

In embodiments in which the operation device is a hydraulic operation device, controller 300 may comprise, or be configured to receive input from, a sensor configured to sense a hydraulic pressure. Hydraulic pressure can be measured to avoid excessive hydraulic pressure which may damage medical device or the tissue of the patient. Excessive hydraulic pressure may also be an indicator that something is wrong with the medical device 10 and may be used for triggering an alarm function for alerting the patient or physician.

Having a plurality of smaller medical devices 10a, 10b, such as shown in FIG. 32, facilitates the insertion of the medical devices 10a, 10b into the body of the patient in either open surgery, laparoscopic surgery or natural orifice transluminal endoscopic surgery (NOTES) using for example a gastroscope.

Although FIG. 32A describes the implantation and operation of two medical devices of the embodiment shown in FIGS. 28A—28C, in alternative embodiments, three, four or more medical devices could be fixated and operated in the same manner. Also, the medical device of FIGS. 29A—29C, 30A—30C or 31A-31B could be operated by a remote unit in the same manner as described with reference to FIG. 32A.

Further features and functions of the remote unit 140, the controller 300 and the implantable energy storage unit 40 are further described with reference to FIGS. 65A—65FH.

FIG. 32B shows a frontal view of the abdomen of the patient when the medical device 10 according to the embodiment described with reference to FIGS. 9 and 10 has been implanted. This is however only an example of an embodiment and it is clear that any of the embodiments of the medical device disclosed herein can be implanted and connected in the manner described with reference to FIG. 32B. The medical device 10 is in the embodiment shown in FIG. 32B operated by a remote unit 140 which in the embodiment shown in FIG. 32B is the remote unit 140 of the embodiment of FIG. 11. This is however only an example of a remote unit for operation of the medical device 10 and it is clear that any of the embodiments of remote units disclosed herein can be implanted and connected in the manner described with reference to FIG. 32B. The remote unit 140 comprises a first portion 141′, a second portion 141″, and a connecting portion 142, mechanically connecting the first and second portions 141′,141″.

The second portion 141″ is in the embodiment shown in FIG. 32B placed on the inside of muscular tissue MT of the abdominal wall AW of the patient, whereas the first portion 141′ is placed on the outside of the muscular tissue MT of the abdominal wall AW, in the subcutaneous tissue ST. As such, the connecting portion 142 travels through a created hole in, or natural orifice between, the muscles of the muscular tissue MT. A cross-sectional area of the connecting portion 142, in a plane in the extension of the muscular tissue MT is smaller than a cross-sectional area of the first and second portions 141′,141″ parallel to the cross-sectional area of the connecting portion 142. The cross-sectional areas of the first and second portions 141′,141″ are also larger than the created hole or natural orifice though which the connecting portion 142 is placed. As such, the first and second portions 141′,141″ are unable to pass through the created hole or natural orifice and is as such fixated to the muscular tissue MT of the abdominal wall. This enables the remote unit 140 to be suspended and fixated to the muscle tissue MT of the abdominal wall AW.

In the embodiment shown in FIG. 32B, the connecting portion 142, is a connecting portion 142 having a circular cross-section and an axial direction AD extending from the first portion 141′ to the second portion 141″. The plane in the extension of the muscular tissue MT, is in the embodiment of FIG. 32B perpendicular to the axial direction AD of the connecting portion 142 extending from the first portion 141′ to the second portion 141″.

As is further described with reference to FIG. 7, in the embodiment of FIG. 32B, the controller is placed in the second portion 141″, and the implantable energy storage unit is placed in the first portion 141′. The controller and the implantable energy storage unit are electrically connected to each other by means of a lead running in the connecting portion 142, such that electrical energy and communication can be transferred from the second 141″ to the first portion 141′, and vice versa. In the embodiment of FIG. 32B, the first portion 141′ further comprises a wireless energy receiver for receiving wireless energy for charging the implantable energy storage unit and/or for powering the medical device 10, and a transceiver for receiving and/or transmitting wireless signals to/from the outside the body. Further features and functions of the controller and the implantable energy storage unit are further described with reference to FIGS. 65A—65FH.

The abdominal wall AW is most locations generally formed by a set of layers of skin, fat/fascia, muscles and the peritoneum. The deepest layer in the abdominal wall AW is the peritoneum PT, which covers many of the abdominal organs, for example the large and small intestines. The peritoneum PT is a serous membrane composed of a layer of mesothelium supported by a thin layer of connective tissue and serves as a conduit for abdominal organ's blood vessels, lymphatic vessels, and nerves. The area of the abdomen enclosed by the peritoneum PT is called the intraperitoneal space. The tissue and organs within the intraperitoneal space are called “intraperitoneal” (e.g., the stomach and intestines). The tissue and organs in the abdominal cavity that are located behind the intraperitoneal space are called “retroperitoneal” (e.g., the kidneys), and tissue and organs located below the intraperitoneal space are called “subperitoneal” or “infraperitoneal” (e.g., the bladder).

The peritoneum PT is connected to a layer of extraperitoneal fat EF which is connected to a layer or transversalis fascia TF. Connected to the transversalis fascia TF, at the area of the abdominal wall AW at which the section is extracted, is muscle tissue MT separated by layers of deep fascia DF.

The deep fascia DF between the layers of muscle is thinner than the transversalis fascia TF and the Scarpa's fascia SF placed on the outside of the muscle tissue MT. Both the transversalis fascia TF and the Scarpa's fascia SF are relatively firm membranous sheets. At the area of the abdominal wall AW at which the section is extracted, the muscle tissue MT is composed of the transverse abdominal muscle TM (transversus abdominis), the internal oblique muscle IM (obliquus internus) and the external oblique muscle EM (obliquus externus). In other areas of the abdominal wall AW, the muscle tissue could also be composed of the rectus abdominis and the pyramidalis muscle.

The layer outside of the muscle tissue MT, beneath the skin SK of the patient is called subcutaneous tissue ST, also called the hypodermis, hypoderm, subcutis or superficial fascia. The main portion of the subcutaneous tissue ST is made up of Camper's fascia which consists primarily of loose connective tissue and fat. Generally, the subcutaneous tissue ST contains larger blood vessels and nerves than those found in the skin.

Placing the remote unit 140 at an area of the abdomen is advantageous as the intestines are easily displaced for making sufficient room for the remote unit 140, without the remote unit 140 affecting the patient too much in a sensational or visual way. Also, the placement of the remote unit 140 in the area of the abdomen makes it possible to fixate the remote unit 140 to the muscle tissue MT of the abdomen for creating an attachment keeping the remote unit 140 firmly in place. In the embodiment shown in FIG. 32B, the first portion 141′ of the remote unit 140 is placed on the left side of the patient in between the peritoneum PT and the muscle tissue MT. The first portion 141′ is placed in the subcutaneous tissue ST between the muscle tissue MT and the skin SK of the patient. Placing the first portion 141′ subcutaneously enables easy access to the first portion 141′ for e.g. wireless communication using a wireless transceiver placed in the first portion 141′, wireless charging of an implantable storage unit using a wireless energy receiver placed in the first portion 141′, injection of a hydraulic fluid (relevant when the operation device is a hydraulic operation device), into an injection port placed in the first portion 141′, manual manipulation of for example a push button placed in the first portion 141′, or maintenance or replacement of the first portion 141′ via a small incision in the skin SK at the first portion 141′.

In the embodiment shown in FIG. 32B, the flexible wires 135 running inside of protective a cover 136 transports linear mechanical force from the remote unit 140 to the main portion M of the medical device 10. The flexible wires 135 run between the peritoneum PT and the muscle tissue MT vertically until the flexible wires 135 reaches the height of the main portion M of the medical device 10.

At this height, the wires 135 enters the peritoneum PT and travels substantially horizontally to the main portion M of the medical device 10. As such, the flexible wire 135 is placed inside of the intraperitoneal space for as short distance as possible which reduces the risk that implanted, foreign body, elements disturb the intraperitoneal organs, reducing the risk of damage to organs, and reducing the risk that foreign body elements cause ileus.

In the embodiment shown in FIG. 32B, the connecting portion 142 connects the first and second portions 141′,141″ though three layers of muscle tissue MT, namely tissue of the transverse abdominal muscle TM, the internal oblique muscle IM and the external oblique muscle EM. In alternative embodiments, it is however conceivable that the second portion 141″ is placed in between layers of muscle, such as between tissue of the transverse abdominal muscle TM, the internal oblique muscle IM, or between the internal oblique muscle IM and the external oblique muscle EM. As such, it is conceivable that in alternative embodiments, the connecting portion 142 connects the first and second portions 141′,141″ through two layers of muscle tissue MT, or through one layer of muscle tissue MT.

In alternative embodiments, it is furthermore conceivable that the first portion 141′ is placed in between layers of muscle, such as between tissue of external oblique muscle EM and the internal oblique muscle IM, or between the internal oblique muscle IM and the transverse abdominal muscle TM.

In embodiments in which the medical device is hydraulically remotely operable (such as further described with reference to FIG. 15), the flexible wires 135 running inside of protective a cover 136 for transporting linear mechanical force from the remote unit 140 to the main portion M shown in FIG. 32B is replaced by conduits (109 in FIG. 15) for conducting hydraulic fluid for transferring force from a portion of the hydraulic operation device placed in the remote unit 140 to a portion of the operation device placed in the main portion M of the medical device 10 hydraulically.

The medical devices of any of the embodiments shown in FIGS. 1A, 1B, 2A, 3A, 4A, 5, 6, 7, 8, 9, 11, 12A, 13A, 14A, 14B, 14C, 14D, 15, 17A, 21, 22, 23, 24, 25, 26, 27, 28A, 29A, 30A, 31a, 31C, 31D, 31F, 31G, 31H, 31I, 31J, 31K, 31K′, 31L, 31L′, 31M, 31N, 31O, 31P, 31Q-31T, 31U, 31V, 31X, 31Y, 33A-33H and 34A-341 could be implanted in the body using open surgery or laparoscopic surgical technique. The laparoscopic surgical technique could comprise the following steps: introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,

blowing in pressurized gas through the needle or trocar or surgical instrument or any device inflating the abdominal cavity,

introducing working instruments comprising at least one of the steps of,

inserting at least one first trocar into the abdominal cavity,

introducing at least one camera through the trocar,

inserting at least a second trocar into the abdominal cavity,

inserting at least one instrument preferably through the second trocar,

inserting at least a third trocar into the abdominal cavity,

inserting at least one second instrument preferably through the third trocar,

dissecting an area of the stomach or intestine,

identifying a suitable position for placing a stretching device on the stomach or intestinal wall,

placing a control device in the body for controlling the stretching device,

inserting the medical device into the abdomen of the patient,

fixating the operable first member to a first portion of the stomach or intestinal wall,

fixating the second member to a second portion of the stomach or intestinal wall,

placing the control unit, and

extracting the instruments, camera and trocar, and in relation thereto

suturing, if necessary, the abdominal wall and permanently closing the skin.

The open surgical technique could comprise the following steps:

cutting and open the skin and abdominal wall.

dissecting or cutting through the fascia of at least one of any muscles or any fibrotic intersections comprising; the rectus abdominalis, transversus abdominalis, the External Oblique, the Internal Oblique, the linea alba, a tendnous intersection, and the umbilicus,

introducing at least one working instrument,

dissecting an area of the stomach,

identifying a suitable position for placing a stretching device on the stomach wall,

placing a control device in the body for controlling the stretching device,

inserting the medical device into the abdomen of the patient,

fixating the operable first member to a first portion of the stomach wall,

fixating the second member to a second portion of the stomach wall,

placing the control unit, and

extracting the instruments, camera and trocar, and in relation thereto

suturing, if necessary, the abdominal wall and permanently closing the skin.

In the following, rotation is to be understood as the motion of an object along a curved path, such as a circular path, around an axis located, at least partially, within the object. Thus, rotation implies no change in the position of the object in relation to the axis. Revolution, instead, is to be understood as the motion of an object along a curved path, such as a circular path, around an axis located outside the object. Thus, revolution implies a change of position of the object in relation to the axis.

FIG. 33A illustrates an embodiment of the medical device 10 for actively stretching a stomach wall of a patient for creating a sensation of satiety. FIG. 33A shows a side view of the embodiment. The medical device 10 comprises a first member 101a, a second member 101b and an operation device 100.

The first member 101a comprises a first member rotating section 101a′ and a first member fixation section 101a″′, which is configured to be fixated to a first portion of the stomach wall. The second member 101b comprises a second member fixation section 101b″′ configured to be fixated to a second portion of the stomach wall. The first member rotating section 101a′ is rotatable around a first axis R1. Each of the first member rotating section 101a′ and operation device 100 is positioned below a first reference plane p1 perpendicular to the first axis R1. The operation device 100 is coupled to the first member rotating section 101a′ and configured to induce a rotation of the first member rotating section 101a′ around the first axis R1. The first member fixation section 101a″′ is configured to describe a revolution around the first axis R1 between a first position and a second position of the first member fixation section 101a″′ in response to the rotation of the first member rotating section 101a′. The second member 101b is coupled to the first member rotating section 101a′ such that, when the first member fixation section 101a″′ revolves towards the second position of the first member fixation section 101a″′, the stomach wall between the first portion and second portion is increasingly stretched.

The rotation of the first member rotating section 101a′ around the first axis R1 is advantageous because, when a fibrotic tissue has encapsulated the first member rotating section 101a′, stretching the stomach wall between the first portion and second portion does not require bending of the fibrotic tissue. This is important because the fibrotic tissue is substantially inelastic, thus being not easily bendable. The first member rotating section 101a′ may easily rotate around the first axis R1 while encapsulated by the fibrotic tissue, which does not hinder the rotation.

The medical device 10 further comprises a main portion M, and the operation device 100 comprises a local portion placed in the main portion M. The main portion M may be configured to be at least partially invaginated by a tissue of the stomach wall (cf. FIGS. 33G-33H) in order to improve stability and long-term fixation of the medical device 10. The local portion of the operation device 100 is coupled to the first member rotating section 101a″.

In the embodiment of FIG. 33A, the first axis R1 is substantially perpendicular to the operation device 100 where the local portion of the operation device 100 is coupled to the first member rotating section 101a′. The first member fixation section 101a″′ is positioned above the first reference plane ρ_1 for the entirety of the revolution of the first member fixation section 101a′. However, in other embodiments the first axis R1 is arranged at an angle in relation to the operation device 100 where the portion of the operation device 100 is coupled to first member rotating section 101a′, said angle being substantially different from ninety degrees. As a result, the first member fixation section 101a″′ may be positioned below the first reference plane ρ_1 for a part of the revolution.

The first member rotating section 101a′ may be configured to be at least partially invaginated by a tissue of the stomach wall (cf. FIGS. 33G-33H) in order to improve stability and long-term fixation of the medical device 10. In the embodiment of FIG. 33A, each of the first member rotating section 101a′ and first member fixation section 101a″′ is a rod having a length and a circular cross-section area, the circular cross-section area having a center. The first axis R1 is a rotational symmetry axis of the first member rotating section 101a′ parallel to the length of the first member rotating section 101a′ and passing by the center of the circular cross-section area of the first member rotating section 101a′. However, each of the first member rotating section 101a′ and first member fixation section 101a″′ may alternatively have a different shape.

In the embodiment of FIG. 33A, the first member rotating section 101a′ is connected to the first member fixation section 101a″′ by a first member coupling section 101a″. The first member coupling section 101a″ is a rod having a circular cross-section area and is positioned above the first reference plane ρ_1. The first member coupling section 101a″ is arranged at angle in relation to the first axis R1. However, the first member coupling section 101a″ may have a different shape, e.g. a curved or serpentine shape, and it may be positioned, at least partially, below the first reference plane ρ1. The first member coupling section 101a″ spaces the first member fixation section 101a″′ apart from the first axis R1. Thus, the first axis R1 is external to the first member fixation section 101a″′, which is configured to revolve around the first axis R1. Preferably, the first member fixation section 101a″′ is parallel to the first member rotating section 101a′. However, the first member fixation section 101a″′ may have a different orientation in relation to the first member rotating section 101a′.

In the embodiment in FIG. 33A, the second member 101b is inoperably fixated to the main portion M. Therefore, the first member rotating section 101a′ and the second member 101b are coupled by the main portion M, which houses the local portion of the operation device 100 coupled to the first member rotating section 101a′. The second member 101b may be configured to be at least partially invaginated by a tissue of the stomach wall in order to improve stability and long-term fixation of the medical device 10. In the embodiment in FIG. 33A, the second member 101b is a rod having a circular cross-section area and is parallel to the first member rotating section 101a′. However, the second member 101b may have a different shape, or a different orientation. Preferably, as shown for the embodiment in FIG. 33A, the second member fixation section 101b′ is arranged at a distance from the main portion M in the direction of the first axis R1, the distance being substantially the same as the distance of the first member fixation section 101a″′ from the main portion M in the direction of the first axis R1. Thus, the stomach wall between the first portion and second portion is free of fibrotic tissue. As a result, the stretching of the stomach wall between the first portion and second portion is facilitated, and the induced sensation of satiety is enhanced. Preferably, as illustrated for the embodiment in FIG. 33A, the second member fixation section 101b″′ is parallel to the first member fixation section 101a″′. Thus, the stretching of the stomach wall between the first portion and second portion is uniform.

In the embodiment in FIG. 33A, the first member fixation section 101a″′ is further configured to describe the revolution around the first axis R1 maintaining a fixed distance 101ap from the first axis R1. The first axis R1 is perpendicular to the main portion M, and the first member fixation section 101a″′ is parallel to the first member rotating section 101a′. Therefore, the first member fixation section 101a″′ is configured to revolve around the first axis R1 by describing a circular path with a radius 101ap. In FIG. 33A, the radius 101ap is the distance between the first axis R1 and the center of the circular cross-section area of the first member fixation section 101a″′.

In the embodiment in FIG. 33A, the first member fixation section 101a″′ is configured to be directly or indirectly fixated to a first portion of the stomach or intestinal wall, and is configured to be invaginated and held in place by tissue-to-tissue sutures or staplers. The second member fixation section 101b″′ is configured to be directly or indirectly fixated to a second portion of the stomach or intestinal wall, and is configured to be invaginated and held in place by tissue-to-tissue sutures or staplers. The operation device 100 is configured to move at least a portion of the first member fixation section 101a″′ in a first direction away from the second member fixation section 101b″′, for stretching the stomach or intestinal wall at least in a stretching plane SP1, SP2 extending between the first member fixation section 101a″′ and second member fixation section 101b″′, wherein a portion of the first member fixation section 101a″′ is positioned in the stretching plane SP1, SP2, and wherein a length of a distance dSP1 between the first member fixation section 101a″′ and the second member fixation section 101b″′ in the stretching plane is less than one half of the length of a circumference CY of the portion of the first member fixation section 101a″′ positioned in the stretching plane and less than the cross-sectional distance dSP2 in the stretching plane SP1, SP2, being equal to the width of the first member fixation section 101a″′. As such, the area available for stretching SP1 exceeds the area of the stomach or intestinal wall used for the invagination of the first member fixation section 101a″′. However, in other embodiments the length of the distance between the first member fixation section 101a″′ and the second member fixation section 101b″′ in the stretching plane may be less than one half of the length of the circumference CY of the portion of the first member fixation section 101a″′ positioned in the stretching plane and/or less than the cross-sectional distance dSP2 in the stretching plane SP1, SP2, being equal to the width of the first member fixation section 101a″′, which may be advantageous in embodiments in which a lot of stomach or intestinal wall is needed to create a stable invagination.

In the embodiment in FIG. 33A, the first member fixation section 101a″′ is configured to be directly or indirectly fixated to a first portion of the stomach or intestinal wall, and is configured to be invaginated and held in place by tissue-to-tissue sutures or staplers. The second member fixation section 101b″′ is configured to be directly or indirectly fixated to a second portion of the stomach or intestinal wall, and is configured to be invaginated and held in place by tissue-to-tissue sutures or staplers. The operation device is configured to move at least a portion of the first member fixation section 101a″′ in a first direction away from the second member fixation section 101b″′, for stretching the stomach or intestinal wall at least in a stretching plane extending between the first member fixation section 101a″′ and second member fixation section 101b″′, wherein a portion of the first member fixation section 101a″′ is positioned in the stretching plane SP1, SP2, and wherein an area SP1 of the stretching plane SP1, SP2 between the first member fixation section 101a″′ and second member fixation section 101b″exceeds an area SP2 of the portion of the first member fixation section 101a″′ positioned in the stretching plane SP1, SP2. As such, the area available for stretching SP1 exceeds the area of the stomach or intestinal wall used for the invagination of the first member fixation section 101a″′. However, in other embodiments the area SP1 of the stretching plane SP1, SP2 between the first member fixation section 101a″′ and second member fixation section 101b″′ may be less than the area SP2 of the portion of the first member fixation section 101a″′ positioned in the stretching plane SP1, SP2, which may be advantageous in embodiments in which a lot of stomach or intestinal wall is needed to create a stable invagination.

In the embodiments shown in FIGS. 33A-33F, the stomach or intestinal wall portion between the first and second member can be undissected and devoid of any implanted parts, such that the stomach or intestinal wall portion between the first and second member can be stretched without interference from fibrotic tissue, which creates a good stretching results.

The local portion of the operation device 100 placed in the main portion M comprises a first conversion device. In FIG. 33A, the first conversion device is a first pulley 144ap. However, in any one of the embodiments herein disclosed the first conversion device may be a different conversion device, e.g. a lever or any type of conversion device adapted to be actuated by a hydraulic system. The first conversion device comprises a receiving portion, which is configured to receive an input local force, and a transmission portion, which is coupled to the receiving portion. The transmission portion is configured to transform the input local force into an output local force inducing a rotation of the first member rotating section 101a′ around the first axis R1.

A top view of the embodiment in FIG. 33A is illustrated in FIG. 33A′. In FIG. 33A′, the first pulley 144ap is also shown. The operation device 100 further comprises a flexible wire 135. The receiving portion of the first pulley 144ap is a rim 144ar, configured to engage with a flexible wire 135.

The transmission portion is a central portion 144ac, which is coupled to the first member rotating section 101a′. The flexible wire 135 is configured to induce a rotation of the first pulley 144ap. The flexible wire 135 is configured to move with an input local speed, and to apply an input local force to the first pulley 144ap. Preferably, the flexible wire 135 is pulled in order to induce a rotation of the first pulley 144ap. When the rim 144ar rotates around the first axis R1 in response to an input local force applied via the flexible wire 135, the central portion 144ac also rotates in the same rotating direction. Thus, the central portion 144ac transmits the rotating movement to the first member rotating section 101a′. The receiving portion is arranged at a distance from the transmission portion, said distance being a characteristic distance of the first conversion device. In FIG. 33A′, the rim 144ar is circular and the first pulley 144ap has a radius 144ap, which is the characteristic distance of the first pulley 144ap. However, other shapes for the rim 144ar are possible, such as an elliptical shape. In alternative embodiments, where the first conversion device is a lever, the receiving portion is an arm of the lever, the arm having a length, and the characteristic distance is the length of the arm.

The radius 144ap is in a range of 0.3 times to 2 times as compared to the distance 101ap of the first member fixation section 101a″′ from the first axis R1. In other embodiments, the radius 144ap is in a range of 0.5 times to 1.2 times as compared to the distance 101ap of the first member fixation section 101a″′ from the first axis R1. In yet other embodiments, the radius 144ap is in a range of 0.95 times to 1.05 times as compared to the distance 101ap of the first member fixation section 101a′ from the first axis R1. The ratio of the radius 101ap over the radius 144ap is the ratio of the input local force applied via the flexible wire 135 to rotate the first pulley 144ap over the force applied in response by the first member fixation section 101a″′ to stretch the stomach wall. Likewise, the ratio of the radius 101ap over the radius 144ap is the ratio of the speed at which the first member fixation section 101a″′ revolves around the first axis R1 over the speed at which the first pulley 144ap rotates around the first axis R1. As a result, by reducing the radius 144ap as compared to the radius 101ap, a smaller input local force needs to be applied via the flexible wire 135 in order for the first member fixation section 101a″′ to stretch the stomach wall with a given stretching force. Likewise, by reducing the radius 144ap as compared to the radius 101ap, the flexible wire 135 needs to move with a larger input local speed in order for the first member fixation section 101a″′ to stretch the stomach wall with a given stretching speed. Preferably, the stomach wall is stretched by applying to the first portion of the stomach wall a stretching force of 5 N and stretching the first portion of the stomach wall at a speed of 2 m/s.

As shown in FIG. 33A′, the flexible wire 135 enters the main portion M by passing through a cover 136 and being subsequently threated over a pulley 144ap′. Finally, the flexible wire 135 engages with the rim 144ar of the first pulley 144ap. The pulley 144ap′ is rotatably fixated to the main portion M.

The first pulley 144ap and the pulley 144ap′ are configured to rotate in opposite rotating directions. In other embodiments, a torsion spring is coupled to the first pulley 144ap in order to bias the first pulley 144ap in a given rotating direction. The use of the pulley 144ap′ as shown in FIG. 33A′ is advantageous to arrange the flexible wire 135 inside the main portion M. However, in any of the embodiments herein disclosed the pulley 144ap′ may be absent.

FIG. 33A″ shows atop view of an embodiment similar to that of FIGS. 33A-33A′. The operation device 100 comprises two flexible wires 135. The first flexible wire 135 enters the main portion by passing through a first cover 136, and being subsequently threated over a pulley 144ap′ and then a pulley 144ap″, the pulley 144ap′ and pulley 144ap″ belonging to a first set and arranged on a first side in the main portion M. Finally, the first flexible wire 135 engages with the rim 144ar of the first pulley 144ap from a first side of the rim 144ar. The second flexible wire 135 enters the main portion by passing through a second cover 136, and being subsequently threated over a pulley 144ap′ and then a pulley 144ap″, the pulley 144ap′ and pulley 144ap″ belonging to a second set and arranged on a second side in the main portion M. The second side in the main portion M where the pulley 144ap′ and pulley 144ap″ engaged by the first flexible wire 135 are arranged is opposite to the first side in the main portion M where the pulley 144ap′ and pulley 144ap″ engaged by the second flexible wire 135 are arranged. Finally, the second flexible wire 135 engages with the rim 144ar of the first pulley 144ap from a second side of the rim 144ar, opposite to the first side of the rim 144ar engaged by the first flexible wire 135.

The pulleys 144ap′ and pulley 144ap″ are rotatably fixated to the main portion M. The two flexible wires 135 are configured to rotate the first pulley 144ap in opposite rotating directions. When the first flexible wire 135 is displaced by a distance in a displacement direction for rotating the first pulley 144ap, the second flexible wire 135 is displaced by the same distance in the opposite displacement direction. Likewise, when the second flexible wire 135 is displaced by a distance in a displacement direction for rotating the first pulley 144ap, the first flexible wire 135 is displaced by the same distance in the opposite displacement direction. Preferably, the first flexible wire 135 is pulled in a pulling direction for rotating the first pulley 144ap in a first rotating direction. Preferably, the second flexible wire 135 is pulled in the same pulling direction for rotating the first pulley 144ar in a second rotating direction opposite to the first rotating direction. When the any of the two flexible wires 135 is displaced, the pulleys 144ap″ rotate in the same rotating direction as the first pulley 144ap, and the pulleys 144ap′ rotate in the opposite rotating direction. The use of two sets of pulleys 144ap′, 144ap″ as shown in FIG. 33A″ is advantageous to arrange the flexible wires 135 inside the main portion M. However, in any of the embodiments herein disclosed any one of the pulleys 144ap′ and 144ap″ may be absent.

FIG. 33B illustrates a side view of an embodiment similar to that in FIGS. 33A-33A′-33A″.

FIG. 33B′ shows a top view of the embodiment in FIG. 33B. The embodiment in FIGS. 33B-33B′ differs from that in FIG. 33A″ in that a single flexible wire 135 engages with the rim of the first pulley 144ap. Similarly to the embodiment in FIG. 33A″, the flexible wire 135 enters the main portion M by passing through a cover 136, and being subsequently threated over a pulley 144ap′ and then a pulley 144ap″.

The pulley 144ap′ and pulley 144ap″ are both arranged on a side of the main portion M, and rotatably fixated to the main portion M. The same considerations made for the embodiment in FIG. 33A″ on the rotating directions of the pulley 144ap′, pulley 144ap″ and first pulley 144ap apply to the embodiment in FIGS. 33B-33B′. However, in contrast to the embodiment in FIG. 33A″, the medical device 10 comprises a torsion spring 145a in the embodiment shown in FIGS. 33B-33B′. The torsion spring 145a is coupled to the first pulley 145a and to the main portion M. The flexible wire 135 is configured to rotate the first pulley 144ap in a first rotating direction, whereas the torsion spring 145a is configured to bias the first pulley 145ap in a second rotating direction opposite to the first rotating direction. Preferably, the flexible wire 135 is pulled in order to rotate the first pulley 144ap. A torsion spring 145a may be included in the embodiment in FIGS. 33A-33A′ to bias the first pulley 144ap therein in a given rotating direction.

FIG. 33C illustrates a side view of an embodiment similar to that in FIG. 33B. A top view of the embodiment in FIG. 33C is shown in FIG. 33C′. The embodiment in FIG. 33C additionally comprises a third member 101c. The third member 101c comprises a third member fixation section 101c″′ configured to be fixated to a third portion of the stomach wall. The third member 101c is coupled to the first member rotating section 101a′ such that, when the first member fixation section 101a″ revolves towards the first position of the first member fixation section 101a″, the stomach wall between the first portion and third portion is increasingly stretched. Like the embodiment in FIGS. 33B-33B′, the embodiment in FIGS. 33C-33C′ comprises atorsion spring 145a, configured to bias the first pulley 144ap in a given rotating direction, and a single flexible wire 135, configured to rotate the first pulley 144ap in the opposite rotating direction.

In the embodiment in FIG. 33C, the second member 101b is inoperably fixated to the main portion M. Therefore, the first member rotating section 101a′ and the third member 101c are coupled by the main portion M, which houses the local portion of the operation device 100 coupled to the first member rotating section 101a′. The third member 101c may be configured to be at least partially invaginated by a tissue of the stomach wall in order to improve stability and long term fixation of the medical device 10. In the embodiment in FIG. 33C, the third member 101c is a rod having a circular cross-section area and is parallel to the first member rotating section 101a′. However, the third member 101c may have a different shape, or a different orientation.

Preferably, as shown for the embodiment in FIG. 33C, the third member fixation section 101c″′ is arranged at a distance from the main portion M in the direction of the first axis R1, the distance being substantially the same as the distance of the first member fixation section 101a″′ from the main portion M in the direction of the first axis R1. Thus, the stomach wall between the first portion and third portion is free of fibrotic tissue. As a result, the stretching of the stomach wall between the first portion and third portion is facilitated, and the induced sensation of satiety is enhanced. Preferably, as illustrated for the embodiment in FIG. 33C, the third member fixation section 101c″′ is parallel to the first member fixation section 101a″′. Thus, the stretching of the stomach wall between the first portion and third portion is uniform.

In the embodiment in FIGS. 33C-33C′, each the first member 101a, second member 101b and third member 101c protrudes in the direction of the first axis R1 from the main portion M on the same side of the main portion M. Furthermore, the second member 101b and third member 101c are positioned on opposite sides in relation to the first axis R1. Thus, when the stomach wall between the first portion and second portion is stretched, the stomach wall between the first portion and third portion contracts, and vice versa. However, other arrangements of the first member 101a, second member 101b and third member 101c are possible, as illustrated below.

As a variation of the embodiment in FIGS. 33C-33C′, another embodiment (not shown) is provided. In this alternative embodiment, the second member and third member protrude in the direction of the first axis from the main portion on a first side and second side, respectively, of the main portion, the second side being opposite to the first side. Thus, the second portion and third portion are arranged on the first side and second side of the main portion, respectively. The first member comprises a top section, protruding in the direction of the first axis on the first side of the main portion, and a bottom section, protruding in the direction of the first axis on the second side of the main portion. Thus, the first member comprises a top first member fixation section and a bottom first member fixation section, configured to be fixated, respectively, to a top first portion and a bottom first portion of the stomach wall. Each of the top section and bottom section of the first member may be the first member 101a in FIG. 33D. The second member and third member are positioned on the same side in relation to the first axis. Thus, when the stomach wall between the second portion and the top first portion is stretched, so is the stomach wall between the third portion and the bottom first portion. Alternatively, the second member and third member are arranged on opposite sides in relation to the first axis. Thus, when the stomach wall between the second portion and the top first portion is stretched, the stomach wall between the third portion and the bottom first portion contracts.

FIG. 33D illustrates a side view of an embodiment similar to that in FIGS. 33C-33C′, and to that in FIG. 33A″. FIG. 33D′ shows a top view of the embodiment in FIG. 33D. Like the embodiment in FIGS. 33C-33C′, the embodiment in FIGS. 33D-33D′ comprises a third member 101c in addition to the first member 101a and second member 101b. The same considerations made for the embodiment in FIGS. 33C-33C′ in relation to the first member 101a, second member 101b and third member 101c apply to the embodiment in FIGS. 33D-33D′. Like the embodiment in FIG. 33A″, the embodiment in FIGS. 33D-33D′ comprises two flexible wires 135 configured to rotate the first pulley 144ap in opposite rotating directions. Like the embodiment in FIG. 33A″, the embodiment in FIGS. 33D-33D′ further comprises the pulleys 144ap′ and pulleys 144ap″. The same considerations made for the embodiment in FIG. 33A″ in relation to the flexible wires 135, the first pulley 144ap, pulleys 144ap′ and pulleys 144ap″ apply to the embodiment in FIGS. 33D-33D′.

FIG. 33E shows a side view of an embodiment similar to that in FIG. 33A. FIG. 33E′ illustrates a top view of the embodiment in FIG. 33E. In the embodiment in FIG. 33E, the first member 101a is the same as in FIG. 33A. Thus, the same considerations made on the first member 101a with reference to FIG. 33A apply to the embodiment in FIG. 33E. The embodiment in FIG. 33E comprises an operation device 100 and a second member 101b. The second member 101b comprises a second member rotating section 101b′ and the second member fixation section 101b″′, which is configured to be fixated to the second portion of the stomach wall. The second member rotating section 101b′ is rotatable around a second axis R2. The first member 101a is the same as in FIGS. 33A-33B. Thus, the same considerations made on the first member 101a with reference to FIGS. 33A-33B apply to the embodiment in FIG. 33E. Likewise, the local portion of the operation device 100 is placed in the main portion M of the medical device 10.

In the embodiment in FIG. 33E, each of the first member rotating section 101a′ and operation device 100 is positioned below the first reference plane ρ_1 perpendicular to the first axis R1. Furthermore, each of the second member rotating section 101b′ and operation device 100 is positioned below a second reference plane ρ_2 perpendicular to the second axis R2. The operation device 100 is coupled to both the first member rotating section 101a′ and to the second member rotating section 101b′.

The operation device 100 is configured to induce a rotation of the first member rotating section 101a′ as disclosed with reference to FIG. 33A. In the embodiment in FIG. 33E, the operation device 100 is further configured to induce a rotation of the second member rotating section 101b′ around the second axis R2.

The second member fixation section 101b″′ is configured to describe a revolution around the second axis R2 between a first position and a second position of the second member fixation section 101b″′ in response to the rotation of the second member rotating section 101b′. The second member rotating section 101b′ is coupled to the first member rotating section 101a′ such that, when the first member fixation section 101a″′ revolves towards the second position of the first member fixation section 101a″′, the stomach wall between the first portion and second portion is increasingly stretched. Furthermore, when the second member fixation section 101b″′ revolves towards the first position of the second member fixation section 101b″′, the stomach wall between the first portion and second portion is increasingly stretched.

The rotation of the second member rotating section 101b′ around the second axis R2 is advantageous because, when a fibrotic tissue has encapsulated the second member rotating section 101b′, stretching the stomach wall between the first portion and second portion does not require bending of the fibrotic tissue. This is important because the fibrotic tissue is substantially inelastic, thus being not easily bendable. The second member rotating section 101b′ may easily rotate around the second axis R2 while encapsulated by the fibrotic tissue, which does not hinder the rotation. Furthermore, each of the first member fixation section 101a″′ and second member fixation section 101b″′ may actively stretch the stomach wall between the first portion and second portion, because each of the first member fixation section 101a″′ and second member fixation section 101b″′ is configured to revolve. Thus, a wider range of stretching values is achievable as compared to the embodiments in FIGS. 33A-33B.

The local portion of the operation device 100 placed in the main portion M is coupled to both the first member rotating section 101a′ and to the second member rotating section 101b′. In the embodiment of FIG. 33E, the second axis R2 is substantially perpendicular to the operation device 100 where the local portion of the operation device 100 is coupled to the second member rotating section 101b′. The second member fixation section 101b″′ is positioned above the second reference plane ρ_2 for the entirety of the revolution of the second member fixation section 101b′. However, in other embodiments the second axis R2 is arranged at an angle in relation to the operation device 100 where the local portion of the operation device 100 is coupled to the second member rotating section 101b′, said angle being substantially different from ninety degrees. As a result, the second member fixation section 101b″′ may be positioned below the second reference plane ρ_2 for a part of the revolution.

The second member rotating section 101b′ may be configured to be at least partially invaginated by a tissue of the stomach wall (cf. FIGS. 33G) in order to improve stability and long term fixation of the medical device 10. In the embodiment of FIG. 33E, each of the second member rotating section 101b′ and second member fixation section 101b″′ is a rod having a length and a circular cross-section area, the circular cross-section area having a center. The second axis R2 is a rotational symmetry axis of the second member rotating section 101b′ parallel to the length of the second member rotating section 101b′ and passing by the center of the circular cross-section area of the second member rotating section 101b′. However, each of the second member rotating section 101b′ and second member fixation section 101b″′ may alternatively have a different shape.

In the embodiment of FIG. 33E, the second member rotating section 101b′ is connected to the second member fixation section 101b″′ by a second member coupling section 101b″. The second member coupling section 101b″ is a rod having a circular cross-section area and is positioned above the second reference plane ρ2. The second member coupling section 101b″ is arranged at angle in relation to the second axis R2. However, the second member coupling section 101b″ may have a different shape, e.g. a curved or serpentine shape, and it may be positioned, at least partially, below the second reference plane ρ_2.

The second member coupling section 101b″ spaces the second member fixation section 101b″′ apart from the second axis R2. Thus, the second axis R2 is external to the second member fixation section 101b″′, which is configured to revolve around the second axis R2. Preferably, the second member fixation section 101b″′ is parallel to the second member rotating section 101b′. However, the second member fixation section 101b″′ may have a different orientation in relation to the second member rotating section 101b′.

Preferably, as shown for the embodiment in FIG. 33E, the second member fixation section 101b″′ is arranged at a distance from the main portion M in the direction of the second axis R2, the distance being substantially the same as the distance of the first member fixation section 101a″′ from the main portion M in the direction of the first axis R1. Thus, the stomach wall between the first portion and second portion is free of fibrotic tissue. As a result, the stretching of the stomach wall between the first portion and second portion is facilitated, and the induced sensation of satiety is enhanced. Preferably, as illustrated for the embodiment in FIG. 33E, the second member fixation section 101b′ is parallel to the first member fixation section 101a″′. Thus, the stretching of the stomach wall between the first portion and second portion is uniform. Preferably, as shown for the embodiment in FIG. 33E, the first axis R1 is parallel to the second axis R2. However, other orientations of the first axis R1 in relation to the second axis R2 are possible.

In the embodiment in FIG. 33E, the second member fixation section 101b″′ is further configured to describe the revolution around the second axis R2 maintaining a fixed distance 101bp from the second axis R2. The second axis R2 is perpendicular to the main portion M, and the second member fixation section 101b″′ is parallel to the second member rotating section 101b′. Therefore, the second member fixation section 101b″′ is configured to revolve around the second axis R2 by describing a circular path with a radius 101bp. In FIG. 33E, the radius 101 bp is the distance between the second axis R2 and the center of the circular cross-section area of the second member fixation section 101b″′.

In the embodiment in FIG. 33E, the local portion of the operation device 100 placed in the main portion M comprises both a first conversion device and a second conversion device. In FIG. 33E, the first conversion device is a first pulley 144ap, and the second conversion device is a second pulley 144bp. However, in any one of the embodiments herein disclosed, any one of the first conversion device and second conversion device may be a different conversion device, e.g. a lever or any type of conversion device adapted to be actuated by a hydraulic system. The first conversion device comprises a receiving portion, which is configured to receive an input local force, and a transmission portion, which is coupled to the receiving portion. The transmission portion is configured to transform an input local force into an output local force inducing a rotation of the first member rotating section 101a′ around the first axis R1. Likewise, the second conversion device comprises a receiving portion, which is configured to receive an input local force, and a transmission portion, which is coupled to the receiving portion. The transmission portion is configured to transform an input local force into an output local force inducing a rotation of the second member rotating section 101b′ around the second axis R2. The receiving portion of the first conversion device is arranged at a distance from the transmission portion of the first conversion device, said distance being a characteristic distance of the first conversion device. Likewise, the receiving portion of the second conversion device is arranged at a distance from the transmission portion of the second conversion device, said distance being a characteristic distance of the second conversion device.

Considerations similar to those made on the first conversion device with reference to FIGS. 33A-33A′ apply to the first conversion device in FIG. 33E. Specifically, the same considerations made on the characteristic distance of the first conversion device with reference to FIGS. 33A-33A′ apply to the characteristic distance of the first conversion device in FIG. 33E. Specifically, the same considerations made on the radius 144ap of the first pulley 144ap with reference to FIGS. 33A-33A′ apply to the radius 144ap of the first pulley 144ap in FIG. 33E. Differently from FIGS. 33A-33A′, the first pulley 144ap in FIGS. 33E-33E′ is engaged by two flexible wires 135, like in FIG. 33A″. Like in FIG. 33A″, two sets of pulleys 144ap′, 144ap″ are used to arrange the flexible wires 135 inside the main portion M.

Considerations similar to those made on the first conversion device with reference to FIGS. 33A-33A′ apply to the second conversion device 144bp in FIG. 33E. Specifically, considerations similar to those made on the first pulley 144ap with reference to FIGS. 33A-33A′ apply to the second pulley 144bp in FIG. 33E. In FIG. 33E′, a top view of the first pulley 144ap and second pulley 144bp is shown.

In the following, we refer to the second pulley 144bp. The receiving portion is a rim 144br, configured to engage with a flexible wire 135. The transmission portion is a central portion 144bc, which is coupled to the second member rotating section 101b′. In FIG. 33E′, the rim 144br is circular and the second pulley 144bp has a radius 144bp, which is the characteristic distance of the second pulley 144bp. However, other shapes for the rim 144br are possible, such as an elliptical shape. In alternative embodiments, where the second conversion device is a lever, the receiving portion is an arm of the lever, the arm having a length, and the characteristic distance is the length of the arm.

The radius 144 bp is in a range of 0.3 times to 2 times as compared to the distance 101bp of the second member fixation section 101b″′ from the second axis R2. In other embodiments, the radius 144 bp is in a range of 0.5 times to 1.2 times as compared to the distance 101 bp of the second member fixation section 101b″′ from the second axis R2. In yet other embodiments, the radius 144 bp is in a range of 0.95 times to 1.05 times as compared to the distance 101bp of the second member fixation section 101b″′ from the second axis R2. The ratio of the radius 101 bp over the radius 144 bp is the ratio of the input local force applied via a flexible wire 135 to rotate the second pulley 144bp over the force applied in response by the second member fixation section 101b″′ to stretch the stomach wall. Likewise, the ratio of the radius 101 bp over the radius 144 bp is the ratio of the speed at which the second member fixation section 101b″′ revolves around the second axis R2 over the speed at which the second pulley 144bp rotates around the second axis R2. As a result, by reducing the radius 144 bp as compared to the radius 101bp, a smaller input local force needs to be applied via a flexible wire 135 in order for the second member fixation section 101b″′ to stretch the stomach wall with a given stretching force. Likewise, by reducing the radius 144 bp as compared to the radius 101 bp, a flexible wire 135 needs to move with a larger input local speed in order for the second member fixation section 101b″′ to stretch the stomach wall with a given stretching speed. Preferably, the stomach wall is stretched by applying to the second portion of the stomach wall a stretching force of 5 N and stretching the second portion of the stomach wall at a speed of 2 m/s.

In the embodiment shown in FIGS. 33E-33E′, the first conversion device and second conversion device are configured to be actuated symbiotically, meaning that the actuation of either conversion device implies the actuation of the other. As shown in FIG. 33E′, the first flexible wire 135, after engaging with the rim 144ar of the first pulley 144ap from a first side of the rim 144ar, engages with the rim 144br of the second pulley 144bp from a second side of the rim 144br, opposite to the first side of the rim 144br. Thus, the first flexible wire 135 is configured to rotate the first pulley 144ap and second pulley 144bp in opposite rotating directions. As shown in FIG. 33E′, the second flexible wire 135, after engaging with the rim 144ar of the first pulley 144ap from a second side of the rim 144ar, engages with the rim 144br of the second pulley 144bp from a first side of the rim 144br, opposite to the second side of the rim 144br. Thus, the second flexible wire 135 is configured to rotate the first pulley 144ap and second pulley 144bp in opposite rotating directions. As discussed in relation to FIG. 33A″, when the first flexible wire 135 is displaced by a distance in a displacement direction for rotating the first pulley 144ap and second pulley 144bp, the second flexible wire 135 is displaced by the same distance in the opposite displacement direction. Likewise, when the second flexible wire 135 is displaced by a distance in a displacement direction for rotating the first pulley 144ap and second pulley 144bp, the first flexible wire 135 is displaced by the same distance in the opposite displacement direction. Preferably, the two flexible wires are moved by being pulled.

As a variation of the embodiment in FIGS. 33E-33E′, another embodiment (not shown) is provided. In this alternative embodiment, the first flexible wire is configured to rotate the first pulley and second pulley as disclosed with reference to FIGS. 33E-33E′, whereas the second flexible wire is absent.

The first flexible wire is configured to rotate the first pulley in a first rotating direction of the first pulley, and further configured to rotate the second pulley in a second rotating direction of the second pulley, opposite to the first rotating direction of the first pulley. The first pulley is coupled to a first torsion spring configured to bias the first pulley in a second rotating direction of the first pulley, opposite to the first rotating direction of the first pulley. The second pulley is coupled to a second torsion spring configured to bias the second pulley in a first rotating direction of the second pulley, opposite to the second rotating direction of the second pulley.

As a variation of the embodiment in FIGS. 33E-33E′, another embodiment (not shown) is provided. In this alternative embodiment, the second conversion device is configured to be actuated independently of the first conversion device. Specifically, the second pulley is configured to be actuated independently of the first pulley. A second pair of flexible wires may be configured to engage with the second pulley, independently of a first pair of flexible wires configured to engage with the first pulley. Alternatively, as shown in FIGS. 33A′-33B′-33C′, the first pair of flexible wires configured to engage with the first pulley may be replaced by a single first flexible wire configured to engage with the first pulley. In such a case, the first pulley may be coupled to a first torsion spring configured to bias the first pulley, as shown in FIGS. 33B′-33C′. Likewise, the second pair of flexible wires configured to engage with the second pulley may be replaced by a single third flexible wire configured to engage with the second pulley. In such a case, the second pulley may be coupled to a second torsion spring configured to bias the second pulley.

FIG. 33F illustrates a side view of an embodiment similar to that in FIG. 33E. FIG. 33F′ illustrates a top view of the embodiment in FIG. 33F. In the embodiment in FIG. 33F, the first member 101a is the same as in FIG. 33E. Thus, the same considerations made on the first member 101a with reference to FIG. 33E apply to the embodiment in FIG. 33F. The second member 101b shown in FIG. 33E is replaced, in the embodiment in FIG. 33F, by a second member 101b which is the same as the second member 101b shown in FIG. 33A. Thus, the same considerations made on the second member 101b shown in FIG. 33A apply to the embodiment in FIG. 33F. The embodiment in FIG. 33F comprises an operation device 100. The embodiment in FIG. 33F further comprises a third member 101c. The third member 101c comprises a third member rotating section 101c′ and a third member fixation section 101c″′, which is configured to be fixated to the third portion of the stomach wall. The third member rotating section 101c′ is rotatable around a third axis R3. The operation device 100 is further coupled to the third member rotating section 101c′ and further configured to induce a rotation of the third member rotating section 101c′ around the third axis R3.

In the embodiment in FIG. 33F, each of the first member rotating section 101a′ and operation device 100 is positioned below the first reference plane ρ_1 perpendicular to the first axis R1. Furthermore, each of the third member rotating section 101c′ and operation device 100 is positioned below a third reference plane ρ_3 perpendicular to the third axis R3. The operation device 100 is coupled to both the first member rotating section 101a′ and to the third member rotating section 101c′. The operation device 100 is configured to induce a rotation of the first member rotating section 101a′ as disclosed with reference to FIG. 33A. In the embodiment in FIG. 33F, the operation device 100 is further configured to induce a rotation of the third member rotating section 101c′ around the third axis R3. The third member fixation section 101c″′ is configured to describe a revolution around the third axis R3 between a first position and a second position of the third member fixation section 101c″′ in response to the rotation of the third member rotating section 101c′. The third member rotating section 101c′ is coupled to the second member 101b such that, when the third member fixation section 101c″′ revolves towards the first position of the third member fixation section 101c″′, the stomach wall between the second portion and third portion is increasingly stretched.

The rotation of the third member rotating section 101c′ around the third axis R3 is advantageous because, when a fibrotic tissue has encapsulated the third member rotating section 101c′, stretching the stomach wall between the second portion and third portion does not require bending of the fibrotic tissue. This is important because the fibrotic tissue is substantially inelastic, thus being not easily bendable. The third member rotating section 101c′ may easily rotate around the third axis R3 while encapsulated by the fibrotic tissue, which does not hinder the rotation.

The operation device 100 comprises a local portion placed in the main portion M. The local portion is coupled to both the first member rotating section 101a′ and to the third member rotating section 101c′. In the embodiment of FIG. 33F, the third axis R3 is substantially perpendicular to the operation device 100 where the local portion of the operation device 100 is coupled to the third member rotating section 101c′. The third member fixation section 101c″′ is positioned above the third reference plane ρ_3 for the entirety of the revolution of the third member fixation section 101c′. However, in other embodiments the third axis R3 is arranged at an angle in relation to the operation device 100 where the local portion of the operation device 100 is coupled to the third member rotating section 101c′, said angle being substantially different from ninety degrees. As a result, the third member fixation section 101c″′ may be positioned below the third reference plane ρ_3 for a part of the revolution.

The third member rotating section 101c′ may be configured to be at least partially invaginated by a tissue of the stomach wall (cf. FIGS. 33G) in order to improve stability and long-term fixation of the medical device 10. In the embodiment of FIG. 33F, each of the third member rotating section 101c′ and third member fixation section 101c″′ is a rod having a length and a circular cross-section area, the circular cross-section area having a center. The third axis R3 is a rotational symmetry axis of the third member rotating section 101c′ parallel to the length of the third member rotating section 101c′ and passing by the center of the circular cross-section area of the third member rotating section 101c′. However, each of the third member rotating section 101c′ and third member fixation section 101c″′ may alternatively have a different shape.

In the embodiment of FIG. 33F, the third member rotating section 101c′ is connected to the third member fixation section 101c″′ by a third member coupling section 101c″. The third member coupling section 101c″ is a rod having a circular cross-section area and is positioned above the third reference plane ρ3. The third member coupling section 101c″ is arranged at angle in relation to the third axis R3. However, the third member coupling section 101c″ may have a different shape, e.g. a curved or serpentine shape, and it may be positioned, at least partially, below the third reference plane ρ3.

The third member coupling section 101c″ spaces the third member fixation section 101c″′ apart from the third axis R3. Thus, the third axis R3 is external to the third member fixation section 101c″′, which is configured to revolve around the third axis R3. Preferably, the third member fixation section 101c″′ is parallel to the third member rotating section 101c′. However, the third member fixation section 101c″′ may have a different orientation in relation to the third member rotating section 101c′.

Preferably, as shown for the embodiment in FIG. 33F, the third member fixation section 101c″′ is arranged at a distance from the main portion M in the direction of the third axis R3, the distance being substantially the same as the distance of the second member fixation section 101b″′ from the main portion M in the direction of the third axis R3. Thus, the stomach wall between the second portion and third portion is free of fibrotic tissue. As a result, the stretching of the stomach wall between the second portion and third portion is facilitated, and the induced sensation of satiety is enhanced. Preferably, as illustrated for the embodiment in FIG. 33F, the third member fixation section 101c″′ is parallel to the second member fixation section 101b″′. Thus, the stretching of the stomach wall between the second portion and third portion is uniform. Preferably, as shown for the embodiment in FIG. 33F, the first axis R1 is parallel to the third axis R3. However, other orientations of the first axis R1 in relation to the third axis R3 are possible.

In the embodiment in FIG. 33F, the third member fixation section 101c″′ is further configured to describe the revolution around the third axis R3 maintaining a fixed distance 101cp from the third axis R3. The third axis R3 is perpendicular to the main portion M, and the third member fixation section 101c″′ is parallel to the third member rotating section 101c′. Therefore, the third member fixation section 101c″′ is configured to revolve around the third axis R3 by describing a circular path with a radius 101cp. In FIG. 33F, the radius 101cp is the distance between the third axis R3 and the center of the circular cross-section area of the third member fixation section 101c″′.

In the embodiment in FIG. 33F, the local portion of the operation device 100 placed in the main portion M comprises both a first conversion device and a second conversion device. In FIG. 33F, the first conversion device is a first pulley 144ap, and the second conversion device is a second pulley 144cp. However, any one of the first conversion device and second conversion device may be a different conversion device, e.g. a lever or any type of conversion device adapted to be actuated by a hydraulic system. The first conversion device comprises a receiving portion, which is configured to receive an input local force, and a transmission portion, which is coupled to the receiving portion. The transmission portion is configured to transform an input local force into an output local force inducing a rotation of the first member rotating section 101a′ around the first axis R1. Likewise, the second conversion device comprises a receiving portion, which is configured to receive an input local force, and a transmission portion, which is coupled to the receiving portion. The transmission portion is configured to transform an input local force into an output local force inducing a rotation of the third member rotating section 101c′ around the third axis R3. The receiving portion of the first conversion device is arranged at a distance from the transmission portion of the first conversion device, said distance being a characteristic distance of the first conversion device. Likewise, the receiving portion of the second conversion device is arranged at a distance from the transmission portion of the second conversion device, said distance being a characteristic distance of the second conversion device.

The same considerations made on the first conversion device with reference to FIG. 33E apply to the first conversion device in FIG. 33F. Specifically, the same considerations made on the characteristic distance of the first conversion device with reference to FIG. 33E apply to the characteristic distance of the first conversion device in FIG. 33F. Specifically, the same considerations made on the radius 144ap of the first pulley 144ap with reference to FIG. 33E apply to the radius 144ap of the first pulley 144ap in FIG. 33F. Like in FIG. 33E, two sets of pulleys 144ap′, 144ap″ are used to arrange the flexible wires 135 inside the main portion M.

The same considerations made on the second conversion device with reference to FIG. 33E apply, mutatis mutandis, to the second conversion device in FIG. 33F. Specifically, the same considerations made on the second pulley 144bp with reference to FIG. 33E apply, mutatis mutandis, to the second pulley 144cp in FIG. 33F. In FIG. 33F′, a top view of the first pulley 144ap and second pulley 144cp is shown. In the following, we refer to the second pulley 144cp. The receiving portion is a rim 144cr, configured to engage with a flexible wire 135. The transmission portion is a central portion 144cc, which is coupled to the third member rotating section 101c′. In FIG. 33F′, the rim 144cr is circular and the second pulley 144cp has a radius 144cp, which is the characteristic distance of the second pulley 144cp. However, other shapes for the rim 144cr are possible, such as an elliptical shape. In alternative embodiments, where the second conversion device is a lever, the receiving portion is an arm of the lever, the arm having a length, and the characteristic distance is the length of the arm.

The radius 144cp is in a range of 0.3 times to 2 times as compared to the distance 101cp of the third member fixation section 101c″′ from the third axis R3. In other embodiments, the radius 144cp is in a range of 0.5 times to 1.2 times as compared to the distance 101cp of the third member fixation section 101c″′ from the third axis R3. In yet other embodiments, the radius 144cp is in a range of 0.95 times to 1.05 times as compared to the distance 101cp of the third member fixation section 101c″′ from the third axis R3. The ratio of the radius 101cp over the radius 144cp is the ratio of the input local force applied via a flexible wire 135 to rotate the second pulley 144cp over the force applied in response by the third member fixation section 101c″′ to stretch the stomach wall. Likewise, the ratio of the radius 101cp over the radius 144cp is the ratio of the speed at which the third member fixation section 101c″′ revolves around the third axis R3 over the speed at which the second pulley 144cp rotates around the third axis R3. As a result, by reducing the radius 144cp as compared to the radius 101cp, a smaller input local force needs to be applied via a flexible wire 135 in order for the third member fixation section 101c″′ to stretch the stomach wall with a given stretching force. Likewise, by reducing the radius 144cp as compared to the radius 101cp, a flexible wire 135 needs to move with a larger input local speed in order for the third member fixation section 101c″′ to stretch the stomach wall with a given stretching speed. Preferably, the stomach wall is stretched by applying to the third portion of the stomach wall a stretching force of 5 N and stretching the third portion of the stomach wall at a speed of 2 m/s.

As shown in FIG. 33F′, the first flexible wire 135, after engaging with the rim 144ar of the first pulley 144ap from a first side of the rim 144ar, engages with the rim 144cr of the second pulley 144cp from a second side of the rim 144cr, opposite to the first side of the rim 144cr. Thus, the first flexible wire 135 is configured to rotate the first pulley 144ap and second pulley 144cp in opposite rotating directions. As shown in FIG. 33F′, the second flexible wire 135, after engaging with the rim 144ar of the first pulley 144ap from a second side of the rim 144ar, engages with the rim 144cr of the second pulley 144cp from a first side of the rim 144cr, opposite to the second side of the rim 144cr. Thus, the second flexible wire 135 is configured to rotate the first pulley 144ap and second pulley 144cp in opposite rotating directions. As discussed in relation to FIG. 33A″, when the first flexible wire 135 is displaced by a distance in a displacement direction for rotating the first pulley 144ap and second pulley 144cp, the second flexible wire 135 is displaced by the same distance in the opposite displacement direction. Likewise, when the second flexible wire 135 is displaced by a distance in a displacement direction for rotating the first pulley 144ap and second pulley 144cp, the first flexible wire 135 is displaced by the same distance in the opposite displacement direction. Preferably, the two flexible wires are moved by being pulled.

As a variation of the embodiment in FIGS. 33F-33F′, another embodiment (not shown) is provided. In this alternative embodiment, the first flexible wire is configured to rotate the first pulley and second pulley as disclosed with reference to FIGS. 33F-33F′, whereas the second flexible wire is absent.

The first flexible wire is configured to rotate the first pulley in a first rotating direction of the first pulley, and further configured to rotate the second pulley in a second rotating direction of the second pulley, opposite to the first rotating direction of the first pulley. The first pulley is coupled to a first torsion spring configured to bias the first pulley in a second rotating direction of the first pulley, opposite to the first rotating direction of the first pulley. The second pulley is coupled to a second torsion spring configured to bias the second pulley in a first rotating direction of the second pulley, opposite to the second rotating direction of the second pulley.

As a variation of the embodiment in FIGS. 33F-33F′, another embodiment (not shown) is provided. In this alternative embodiment, the second conversion device is configured to be actuated independently of the first conversion device. Specifically, the second pulley is configured to be actuated independently of the first pulley. A second pair of flexible wires may be configured to engage with the second pulley, independently of a first pair of flexible wires configured to engage with the first pulley. Alternatively, as shown in FIGS. 33A′-33B′-33C′, the first pair of flexible wires configured to engage with the first pulley may be replaced by a single flexible wire configured to engage with the first pulley.

In such a case, the first pulley may be coupled to a first torsion spring configured to bias the first pulley, as shown in FIGS. 33B′-33C′. Likewise, the second pair of flexible wires configured to engage with the second pulley may be replaced by a single flexible wire configured to engage with the second pulley. In such a case, the second pulley may be coupled to a second torsion spring configured to bias the second pulley.

In any one of the embodiments disclosed with reference to FIGS. 33A-33A′-33A″-33B-33B′-33C-33C′-33D-33D′-33E-33E′-33F-33F′, comprising any related variation embodiment, the local portion of the operation device 100 may comprise a first local motor coupled to the receiving portion of the first conversion device, where the first local motor is configured to generate a movement having a first input local force and a first input local speed. Thus, the first local motor is configured to induce a rotation of the first member rotating section 101a′ around the first axis R1. For example, the first local motor may be coupled to the first member rotating section 101a′ by a worm drive, similarly to the motor MO1 which is coupled to the member 101c in FIG. 5. The worm drive comprises a worm and a worm wheel. The first local motor may be a piezoelectric motor, as disclosed with reference to FIGS. 58A-58B-58C-58D-58E-58F-58G-58H. The first conversion device may be a gear system, configured to reduce the first input local speed and increase the first input local force.

In any one of the embodiments disclosed with reference to FIGS. 33E-33E′, comprising any related variation embodiment, the local portion of the operation device 100 may comprise a second local motor coupled to the receiving portion of the second conversion device, where the second local motor is configured to generate a movement having a second input local force and a second input local speed. Thus, the second local motor is configured to induce a rotation of the second member rotating section 101b′ around the second axis R2. For example, the second local motor may be coupled to the second member rotating section 101b′ by a worm drive, similarly to the motor MO1 which is coupled to the member 101c in FIG. 5. The worm drive comprises a worm and a worm wheel. The second local motor may be a piezoelectric motor, as disclosed with reference to FIGS. 58A-58B-58C-58D-58E-58F-58G-58H. The second conversion device may be a gear system, configured to reduce the second input local speed and increase the second input local force.

In any one of the embodiments disclosed with reference to FIGS. 33F-33F′, comprising any related variation embodiment, the local portion of the operation device 100 may comprise a second local motor coupled to the receiving portion of the second conversion device, where the second local motor is configured to generate a movement having a second input local force and a second input local speed. Thus, the second local motor is configured to induce a rotation of the third member rotating section 101c′ around the third axis R3. For example, the second local motor may be coupled to the third member rotating section 101c′ by a worm drive, similarly to the motor MO1 which is coupled to the member 101c in FIG. 5. The worm drive comprises a worm and a worm wheel. The second local motor may be a piezoelectric motor, as disclosed with reference to FIGS. 58A-58B-58C-58D-58E-58F-58G-58H. The second conversion device may be a gear system, configured to reduce the second input local speed and increase the second input local force.

As discussed for other embodiments herein disclosed, in any one of the embodiments disclosed with reference to FIGS. 33A-33A′-33A″-33B-33B′-33C-33C′-33D-33D′-33E-33E′-33F-33F′, comprising any related variation embodiment, the medical device 10 may comprise a remote unit 140 configured to be placed at a remote location in the body of the patient, as shown in FIG. 33H. In such a case, the operation device 100 further comprises a remote portion placed in the remote unit. The remote unit 140 is the remote unit 140 disclosed with reference to FIG. 14D.

In any one of the embodiments, comprising any related variation embodiment, disclosed with reference to FIGS. 33A-33A′-33A″-33B-33B′-33C-33C′-33D-33D′-33E-33E′-33F-33F′, the remote portion of the operation device may further comprise a first remote motor MO1 (cf. FIG. 33H) coupled to the receiving portion of the first conversion device and configured to generate a movement having a first input remote speed and a first input remote force. In such a case, a first flexible wire may be configured to transmit the first input remote force from the first remote motor MO1 to the receiving portion of the first conversion device. The first remote motor MO1 may be a piezoelectric motor, as disclosed with reference to FIGS. 58A-58B-58C-58D-58E-58F-58G-58H. The remote portion of the operation device may further comprise a first remote gear system configured to reduce the first input remote speed and increase the first input remote force. The first remote gear system is coupled to the first remote motor MO1 and to the first flexible wire.

In any one of the embodiments, comprising any related variation embodiment, disclosed with reference to FIGS. 33A″-33D-33D′-33E-33E′-33F-33F′ where the first conversion device may be actuated in at least two opposite directions, the remote portion of the operation device may comprise, in addition to the first remote motor MO1 (cf. FIG. 33H), a second remote motor MO2 (cf. FIG. 33H) coupled to the receiving portion of the first conversion device and configured to generate a movement having a second input remote speed and a second input remote force. In such a case, a second flexible wire may be configured to transmit the second input remote force from the second remote motor MO2 to the receiving portion of the second conversion device. The second flexible wire is configured to induce a rotation of the first member rotating section in a second rotating direction of the first member rotating section opposite to the first rotating direction of the first member rotating section. The second remote motor MO2 may be a piezoelectric motor, as disclosed with reference to FIGS. 58A-58B-58C-58D-58E-58F-58G-58H. The remote portion of the operation device may further comprise a second remote gear system configured to reduce the second input remote speed and increase the second input remote force.

The second remote gear system is coupled to the second remote motor MO2 and to the second flexible wire.

In any one of the embodiments, comprising any related variation embodiment, disclosed with reference to FIGS. 33A″-33D-33D′-33E-33E′-33F-33F′ where the second conversion device is configured to be actuated independently of the first conversion device, the remote portion of the operation device may further comprise a third remote motor coupled to the receiving portion of the second conversion device and configured to generate a movement having a third input remote speed and a third input remote force. In such a case, a third flexible wire may be configured to transmit the third input remote force from the third remote motor to the receiving portion of the second conversion device.

The third remote motor may be a piezoelectric motor, as disclosed with reference to FIGS. 58A-58B-58C-58D-58E-58F-58G-58H. The remote portion of the operation device may further comprise a third remote gear system configured to reduce the third input remote speed and increase the third input remote force.

The third remote gear system is coupled to the third remote motor and to the third flexible wire.

In any one of the embodiments, comprising any related variation embodiment, disclosed with reference to FIGS. 33A″-33D-33D′-33E-33E′-33F-33F′ where the second conversion device is configured to be actuated independently of the first conversion device and where the second conversion device is configured to be actuated in at least two opposite directions, the remote portion of the operation device may comprise, in addition to the third remote motor, a fourth remote motor coupled to the receiving portion of the second conversion device and configured to generate a movement having a fourth input remote speed and a fourth input remote force. In such a case, a fourth flexible wire may be configured to transmit the fourth input remote force from the fourth remote motor to the receiving portion of the second conversion device. The fourth remote motor may be a piezoelectric motor, as disclosed with reference to FIGS. 58A-58B-58C-58D-58E-58F-58G-58H. The remote portion of the operation device may further comprise a fourth remote gear system configured to reduce the fourth input remote speed and increase the fourth input remote force. The fourth remote gear system is coupled to the fourth remote motor and to the fourth flexible wire.

In any one of the embodiments, comprising any related variation embodiment, disclosed with reference to FIGS. 33A-33A′-33A″-33B-33B′-33C-33C′-33D-33D′-33E-33E′-33F-33F′ where the first conversion device is adapted to be actuated by a hydraulic system, the remote portion of the operation device may further comprise a first remote motor coupled to the receiving portion of the first conversion device and configured to generate a movement having a first input remote speed and a first input remote force. In such a case, the operation device may further comprise a first hydraulic system configured to transmit the first input remote force from the first remote motor to the receiving portion of the first conversion device. The first hydraulic system is similar to the hydraulic system disclosed with reference to FIG. 15. The first hydraulic system comprises a first hydraulic conduit and a first remote hydraulic cylinder placed in the remote portion of the operation device. The first remote hydraulic cylinder is coupled to the first remote motor and to the first hydraulic conduit. The first hydraulic system further comprises a first local hydraulic cylinder placed in the local portion of the operation device, the first local hydraulic cylinder being coupled to the first hydraulic conduit and to the receiving portion of the first conversion device. The first remote motor may be a piezoelectric motor, as disclosed with reference to FIGS. 58A-58B-58C-58D-58E-58F-58G-58H. The remote portion of the operation device may further comprise a first remote gear system configured to reduce the first input remote speed and increase the first input remote force. The first remote gear system is coupled to the first remote motor and to the first hydraulic conduit.

In any one of the embodiments, comprising any related variation embodiment, disclosed with reference to FIGS. 33A″-33D-33D′-33E-33E′-33F-33F′ where the first conversion device may be actuated in at least two opposite directions and where it is adapted to be actuated by a hydraulic system, the remote portion of the operation device may comprise, in addition to the first remote motor, a second remote motor coupled to the receiving portion of the first conversion device and configured to generate a movement having a second input remote speed and a second input remote force. In such a case, the operation device may comprise, in addition to the first hydraulic system, a second hydraulic system configured to transmit the second input remote force from the second remote motor to the receiving portion of the first conversion device. The first hydraulic system is configured to induce a rotation of the first member rotating section in a first rotating direction of the first member rotating section. The second hydraulic system is configured to induce a rotation of the first member rotating section in a second rotating direction of the first member rotating section opposite to the first rotating direction of the first member rotating section. The second hydraulic system is similar to the hydraulic system disclosed with reference to FIG. 15. The second hydraulic system comprises a second hydraulic conduit and a second remote hydraulic cylinder placed in the remote portion of the operation device. The second remote hydraulic cylinder is coupled to the second remote motor and to the second hydraulic conduit. The second hydraulic system further comprises a second local hydraulic cylinder placed in the local portion of the operation device, the second local hydraulic cylinder being coupled to the second hydraulic conduit and to the receiving portion of the first conversion device. The second remote motor may be a piezoelectric motor, as disclosed with reference to FIGS. 58A-58B-58C-58D-58E-58F-58G-58H. The remote portion of the operation device may further comprise a second remote gear system configured to reduce the second input remote speed and increase the second input remote force. The second remote gear system is coupled to the second remote motor and to the second hydraulic system.

In any one of the embodiments, comprising any related variation embodiment, disclosed with reference to FIGS. 33A″-33D-33D′-33E-33E′-33F-33F′ where the second conversion device is configured to be actuated independently of the first conversion device and where the second conversion device is adapted to be actuated by a hydraulic system, the remote portion of the operation device may further comprise a third remote motor coupled to the receiving portion of the second conversion device and configured to generate a movement having a third input remote speed and a third input remote force.

In such a case, the operation device may further comprise a third hydraulic system configured to transmit the third input remote force from the third remote motor to the receiving portion of the second conversion device. The third hydraulic system is similar to the hydraulic system disclosed with reference to FIG. 15. In those embodiments where the second conversion device is configured to induce a rotation of the second member rotating section, the third hydraulic system is configured to induce a rotation of the second member rotating section. In those embodiments where the second conversion device is configured to induce a rotation of the third member rotating section, the third hydraulic system is configured to induce a rotation of the third member rotating section. The third hydraulic system comprises a third hydraulic conduit and a third remote hydraulic cylinder placed in the remote portion of the operation device. The third remote hydraulic cylinder is coupled to the third remote motor and to the third hydraulic conduit. The third hydraulic system further comprises a third local hydraulic cylinder placed in the local portion of the operation device, the third local hydraulic cylinder being coupled to the third hydraulic conduit and to the receiving portion of the second conversion device. The third remote motor may be a piezoelectric motor, as disclosed with reference to FIGS. 58A-58B-58C-58D-58E-58F-58G-58H. The remote portion of the operation device may further comprise a third remote gear system configured to reduce the third input remote speed and increase the third input remote force. The third remote gear system is coupled to the third remote motor and to the third hydraulic conduit.

In any one of the embodiments, comprising any related variation embodiment, disclosed with reference to FIGS. 33A″-33D-33D′-33E-33E′-33F-33F′ where the second conversion device is configured to be actuated by a hydraulic system independently of the first conversion device and where the second conversion device is configured to be actuated in at least two opposite directions, the remote portion of the operation device may comprise, in addition to the third remote motor, a fourth remote motor coupled to the receiving portion of the second conversion device and configured to generate a movement having a fourth input remote speed and a fourth input remote force. In such a case, the operation device may comprise, in addition to the third hydraulic system, a fourth hydraulic system configured to transmit the fourth input remote force from the fourth remote motor to the receiving portion of the second conversion device. In those embodiments where the second conversion device is configured to induce a rotation of the second member rotating section, the third hydraulic system is configured to induce a rotation of the second member rotating section in a first rotating direction of the second member rotating section, and the fourth hydraulic system is configured to induce a rotation of the second member rotating section in a second rotating direction of the second member rotating section opposite to the first rotating direction of the second member rotating section. In those embodiments where the second conversion device is configured to induce a rotation of the third member rotating section, the third hydraulic system is configured to induce a rotation of the third member rotating section in a first rotating direction of the third member rotating section, and the fourth hydraulic system is configured to induce a rotation of the third member rotating section in a second rotating direction of the third member rotating section opposite to the first rotating direction of the third member rotating section.

The fourth hydraulic system is similar to the hydraulic system disclosed with reference to FIG. 15. The fourth hydraulic system comprises a fourth hydraulic conduit and a fourth remote hydraulic cylinder placed in the remote portion of the operation device. The fourth remote hydraulic cylinder is coupled to the fourth remote motor and to the fourth hydraulic conduit. The fourth hydraulic system further comprises a fourth local hydraulic cylinder placed in the local portion of the operation device, the fourth local hydraulic cylinder being coupled to the fourth hydraulic conduit and to the receiving portion of the second conversion device. The fourth remote motor may be a piezoelectric motor, as disclosed with reference to FIGS. 58A-58B-58C-58D-58E-58F-58G-58H. The remote portion of the operation device may further comprise a fourth remote gear system configured to reduce the fourth input remote speed and increase the fourth input remote force. The fourth remote gear system is coupled to the fourth remote motor and to the fourth hydraulic system.

In any one of the embodiments herein disclosed comprising both a first remote motor and third remote motor, in addition to both a first flexible wire and third flexible wire, the third flexible wire being configured to induce a rotation of the second member rotating section, the first remote motor may be the same as the third remote motor. In such a case, the first flexible wire is the same as the third flexible wire, and the first flexible wire is configured to rotate the first member rotating section and second member rotating section in opposite rotating directions.

In any one of the embodiments herein disclosed comprising both a first remote motor and third remote motor, in addition to both a first flexible wire and third flexible wire, the third flexible wire being configured to induce a rotation of the third member rotating section, the first remote motor may be the same as the third remote motor. In such a case, the first flexible wire is the same as the third flexible wire, and the first flexible wire is configured to rotate the first member rotating section and third member rotating section in opposite rotating directions.

In any one of the embodiments herein disclosed comprising a first remote motor, a second remote motor, a third remote motor and a fourth remote motor, in addition to a first flexible wire, a second flexible wire, a third flexible wire and a fourth flexible wire, the third flexible wire and fourth flexible wire being configured to induce a rotation of the second member rotating section, the first remote motor may be the same as the third remote motor, and the second remote motor may be the same as the fourth remote motor. In such a case, the first flexible wire is the same as the third flexible wire, and the second flexible wire is the same as the fourth flexible wire. The first rotating direction of the first member rotating section is opposite to the first rotating direction of the second member rotating section, and the second rotating direction of the first member rotating section is opposite to the second rotating direction of the second member rotating section.

In any one of the embodiments herein disclosed comprising a first remote motor, a second remote motor, a third remote motor and a fourth remote motor, in addition to a first flexible wire, a second flexible wire, a third flexible wire and a fourth flexible wire, the third flexible wire and fourth flexible wire being configured to induce a rotation of the third member rotating section, the first remote motor may be the same as the third remote motor, and the second remote motor may be the same as the fourth remote motor. In such a case, the first flexible wire is the same as the third flexible wire, and the second flexible wire is the same as the fourth flexible wire. The first rotating direction of the first member rotating section is opposite to the first rotating direction of the third member rotating section, and the second rotating direction of the first member rotating section is opposite to the second rotating direction of the third member rotating section.

In any one of the embodiments herein disclosed comprising both a first remote motor and third remote motor, in addition to both a first hydraulic system and third hydraulic system, the third hydraulic system being configured to induce a rotation of the second member rotating section, the first remote motor may be the same as the third remote motor. In such a case, the first hydraulic system is the same as the third hydraulic system, and the first hydraulic system is configured to rotate the first member rotating section and second member rotating section in opposite rotating directions.

In any one of the embodiments herein disclosed comprising both a first remote motor and third remote motor, in addition to both a first hydraulic system and third hydraulic system, the third hydraulic system being configured to induce a rotation of the third member rotating section, the first remote motor may be the same as the third remote motor. In such a case, the first hydraulic system is the same as the third hydraulic system, and the first hydraulic system is configured to rotate the first member rotating section and third member rotating section in opposite rotating directions.

In any one of the embodiments herein disclosed comprising a first remote motor, a second remote motor, a third remote motor and a fourth remote motor, in addition to a first hydraulic system, a second hydraulic system, a third hydraulic system and a fourth hydraulic system, the third hydraulic system and fourth hydraulic system being configured to induce a rotation of the second member rotating section, the first remote motor may be the same as the third remote motor, and the second remote motor may be the same as the fourth remote motor. In such a case, the first hydraulic system is the same as the third hydraulic system, and the second hydraulic system is the same as the fourth hydraulic system. The first rotating direction of the first member rotating section is opposite to the first rotating direction of the second member rotating section, and the second rotating direction of the first member rotating section is opposite to the second rotating direction of the second member rotating section.

In any one of the embodiments herein disclosed comprising a first remote motor, a second remote motor, a third remote motor and a fourth remote motor, in addition to a first hydraulic system, a second hydraulic system, a third hydraulic system and a fourth hydraulic system, the third hydraulic system and fourth hydraulic system being configured to induce a rotation of the third member rotating section, the first remote motor may be the same as the third remote motor, and the second remote motor may be the same as the fourth remote motor. In such a case, the first hydraulic system is the same as the third hydraulic system, and the second hydraulic system is the same as the fourth hydraulic system. The first rotating direction of the first member rotating section is opposite to the first rotating direction of the third member rotating section, and the second rotating direction of the first member rotating section is opposite to the second rotating direction of the third member rotating section.

FIG. 34A shows a frontal view of the abdomen of the patient, just as is shown in FIG. 32B. In the embodiment shown in FIG. 34A, a medical device 10 comprising two first members 101a according to the embodiments further described with reference to FIG. 34C has been positioned on and fixated to the proximal portion of the Duodenum, close to the pylorus and antrum region of the stomach, more specifically at the portion of the Duodenum called the Duodenal Flexure. This is however only an example of an embodiment of a stretching device capable of being positioned in the region of the Duodenum and it is clear that many of the embodiments of stretching devices disclosed herein can be implanted and connected in the manner described with reference to FIG. 34A. The medical device 10 is in the embodiment shown in FIG. 34A operated by a remote unit 140 which in the embodiment shown in FIG. 34A is the remote unit 140 of the embodiment of FIG. 11. This is however only an example of a remote unit for operation of the medical device 10 and it is clear that any of the embodiments of remote units disclosed herein can be implanted and connected in the manner described with reference to FIG. 34A.

In the embodiment shown in FIG. 34A, the flexible wires 135 that transports linear mechanical force from the remote unit 140 to the first members 101a of the medical device 10 are running inside of a protective cover. The flexible wires 135 run between the peritoneum PT and the muscle tissue MT horizontally across the abdomen until the flexible wires 135 reaches the position of the first members 101a of the medical device 10. At this position, the wires 135 enters the peritoneum PT and travels substantially dorsally to the first members 101a of the medical device 10. As such, the flexible wires 135 are placed inside of the intraperitoneal space for as short distance as possible which reduces the risk that implanted, foreign body, elements disturb the intraperitoneal organs. This reduces the risk of damage to organs and reduces the risk that foreign body elements cause ileus.

In embodiments in which the medical device is hydraulically remotely operable (such as for example when the first member 101a is a first member of the type described with reference to FIG. 31L′) the flexible wires are replaced by conduits (109 in FIG. 31L) for conducting hydraulic fluid for transferring force from a portion of a hydraulic operation device placed in the remote unit to the first member of the medical device 10 hydraulically.

FIG. 34B shows an embodiment of an implantable medical device 10 for actively stretching an intestinal wall of a human for creating a sensation of satiety. The medical device 10 comprises an operable first member 101a configured to be fixated to a first portion P1 of the intestinal wall IW. In the embodiment shown in FIG. 34B, the operable first member 101a is a curved operable first member, more specifically a curved operable first member 101a forming a closed curve in the form or a circular ring enclosing the first portion P1 of the intestinal wall IW. The curved operable first member 101a is fixated to the first intestine portion P1 by means of invagination. I.e., the intestine is folded or rolled over the ring-shaped first member 101a and then sutured 121 intestinal tissue—to—intestinal tissue, such that the ring-shaped first member 101a becomes placed in an annular tissue “tunnel” extending radially around the circumference of the first intestinal portion P1. The operation device is configured alter the curvature of the first member 101a, i.e. increasing length of the circumference and thereby the diameter of the ring-shaped first member 101a. As the ring-shaped first member 101a is fixated to the first portion of the intestine P1, on the outside thereof, the increasing diameter creates an outward pushing force increasing the diameter of the intestine and thereby actively stretching the portion of the intestine P1 in a radial direction for creating a sensation of satiety. In alternative embodiments, the operable first member 101a may be fixated the intestinal wall IW in other ways, such as with sutures running from the operable first member 101a to the intestinal wall IW. What is important is that the operable first member 101a is fixated to the first portion P1 of the intestinal wall IW at a plurality of positions, such that that the different annularly distributed positions of the intestinal wall expand in different directions, such that the tissue of the intestinal wall placed between the fixation positions becomes stretched as the circumference of the ring-shaped closed curve is expanded.

Generally, a non-therapeutic method of creating a sensation of satiety using a pre-implanted medical device configured to stretch a portion of the stomach wall of a human is further provided. The non-therapeutic method generally comprises altering the operable first member such that the intestinal wall is actively stretched for creating a sensation of satiety.

The non-therapeutic method of creating a feeling of satiety could be linked to a life-style choice of not eating too much or could be linked to a desired physical appearance and as such be linked to physical esthetics.

The first member 101a may comprise or be connected to an implantable controller configured to control the operation of the first member 101a. In such embodiments, the non-therapeutic method comprises operating the first member as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

In the embodiment shown in FIG. 34B, the operable first member 101a is an operable first member according to the embodiment further described with reference to FIG. 34G. However, it is equally conceivable that the first members 101a, according to any of the embodiments described with reference to FIGS. 34E-341, are invaginated and operated in the same way.

In alternative embodiments, at least one stimulation electrode could be placed on the operable first member 101a (in the same way as further described with reference to FIG. 60) of electrically stimulating tissue wall of the intestine. The stimulation electrode could in some embodiments be complemented or replaced by a vibrator configured to mechanically stimulate a wall portion of the stomach, such that the wall of the intestine is actively stretched and electrically and/or mechanically stimulated simultaneously for creating a sensation of satiety. The vibrator may be a vibrator such as further described with reference to FIGS. 64X—64Z and could be configured to be operated by a piezoelectric operation device, such as any of the piezoelectric motors described with reference to FIGS. 58A-58H.

The electrode E1 or vibrator may be connected to a wireless energy receiver configured to receive energy for powering the electrode or vibrator wirelessly.

The operable first member 101a when including an electrode and/or vibrator may be configured to be at least partially invaginated by the tissue of the stomach wall using stomach-to-stomach sutures or staplers.

The vibrator may be configured to vibrate at a frequency in the range of 1-100 Hz, such as in the range of 1-20 Hz, and with a period of 0.01-1 second, such as of 0.05-1 second. The vibrator may be configured to vibrate at an amplitude such that the tissue in the intestine wall is displaced at least 1 mm.

The member 101a may in some embodiments further comprises a second electrode, and the first and second electrodes may be configured for successively electrically stimulating two portions of the tissue wall of the intestine. The first and second electrodes may be placed on the member 101a at a distance from each other being at least 5 mm, and/or a distance from each other corresponding to a wavelength of a wave pattern of a movement of smooth muscles of the intetinal wall, or by a multiple of such a wavelength. The stimulation controller 350 may be configured to send stimulation signals to the first and second electrode in pulses, or pulse trains. And the pulses or pulse trains may have a frequency being determined based on a frequency of the wave pattern of a movement of smooth muscles of the stomach. The stimulation controller 350 is configured to send stimulation signals to the first and second electrode in pulses or pulse trains having a pulse separation or pulse train separation of 0,5-10 s, such as 1-8 s, such as 2-6 s.

FIG. 34C shows an embodiment of the medical device for stretching the intestinal wall IW of the patient for creating a feeling of satiety. In the embodiment shown in FIG. 34C (and in FIG. 34A), the medical device comprises two operable first members 101a, each of which being configured to expand radially such as further described with reference to FIG. 34B. The invaginated first members of FIG. 34C could be any one of the ring-shaped first members described with reference to FIGS. 34E-341. The advantage of having several first members for creating the stretching, is that more total tissue can be stretched, and also the stretched area can be varied such that the effect of actively stretching a tissue portion is not reduce by over stretching or fatigue of a specific tissue portion.

FIG. 34D shows another embodiment of a medical device 10, in which the medical device 10 comprises a first member 101a, being a ring-shaped member, configured to be fixated to a first portion P1 of the intestinal wall IW by means of invagination. The medical device further comprises a second member 101b, being a ring-shaped member, configured to be fixated to a second portion P2 of the intestinal wall IW by means of invagination. The medical device 10 further comprises an operation device in the form of a hydraulic cylinder 143 configured to be operated for moving the first and second members 101a, 101b away from each other, such that the first and second positions P1, P2 are moved away from each other, axially in the direction of the elongation of the intestine, such that the area of the intestinal tissue wall IW between the first and second positions P1, P2 is stretched, such that a sensation of satiety is induced. One advantage of stretching stomach wall between a first and second position, is that the intestinal tissue between the first and second position P1, P2 may be unaffected by fibrotic tissue, as no part of the medical device is placed in direct contact with the tissue wall of the intestine in this area. In alternative embodiments, it is conceivable that the embodiments are combined, such that the members expand radially and away from each other for stretching the tissue wall of the intestine in two directions simultaneously.

Generally, a non-therapeutic method of creating a sensation of satiety using a pre-implanted medical device configured to stretch a portion of the stomach wall of a human is further provided. The non-therapeutic method generally comprises operating the operable first member by moving at least a portion of the operable first member in a first direction away from the second member, for stretching the intestinal wall between the first and second portions of the intestinal wall, for creating a sensation of satiety.

The non-therapeutic method of creating a feeling of satiety could be linked to a life-style choice of not eating too much or could be linked to a desired physical appearance and as such be linked to physical esthetics.

The first member 101a may comprise or be connected to an implantable controller configured to control the operation of the first member 101a. In such embodiments, the non-therapeutic method comprises operating the first member as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

According to one embodiment, the step of operating the operable first member by moving at least a portion of the operable first member in a first direction away from the second member comprises operating the operable first member by moving at least a portion of the operable first member in a first direction away from the second member substantially in an axial direction of the intestine.

According to one embodiment, the method further comprises the step of sending a wireless control signal from a wireless remote control to the pre-implanted medical device. The operable first member is operated as a result of the receipt of the wireless control signal at the pre-implanted medical device. The pre-implanted medical device may further comprise a controller configured to control the operation of the operable first member. The method may further comprise operating the operable first member as a result of at least one of: the receipt of a wireless control signal at the controller of the pre-implanted medical device, the receipt of a sensor signal from a pre-implanted sensor at the controller, and the lapse of a pre-determined time.

In all of the embodiments shown with reference to FIGS. 34B-34D, the operation device may be placed locally, i.e. directly in or on the first members 101a, or may be placed in a remote unit (140), such as shown in FIG. 34A.

In all of the embodiments of FIGS. 34B-34D, the first member may be a first member configured to have a changing volume, such that the first member can expand and increase its volume inside of the tissue tunnel in which it is placed. One example of a first member capable of expanding its volume hydraulically is shown in FIG. 34G.

In embodiments in which the members are configured to be operated by a remote operation device, or an operation device placed partially remotely, the first member may be configured to be completely invaginated by the intestinal wall, apart from elements for transferring force or communication to and from the first member.

FIG. 34E shows an embodiment of a member 101, which may be used as a first member 101a in the embodiments shown with reference to FIGS. 34A-34D. In the embodiment shown in FIG. 34E, the member 101 comprises an inflatable portion 175 encircling the outer periphery of the member 101. The inflatable portion 175 is fluidly connected to a fluid conduit 109 which may connect the inflatable portion 175 to a subcutaneously placed injection port or to a hydraulic pump which may be placed in a remote unit. The inflatable portion 175 is configured to expand and thereby increase the volume and thereby the outer diameter of the member 101, for creating an induced feeling of satiety.

FIG. 34F shows an alternative embodiment, similar to the embodiment shown with reference to FIG. 34E, the difference being that in the embodiment shown in FIG. 31F, the member 101 is operated by a flexible wire 135 configured to transfer a rotating force. The flexible wire 135 runs inside of a protective cover 136. The flexible wire 135 comprises a threaded portion which engages a nut-portion having inner threads such that a nut-and-screw transmission element 138 is created for transferring the rotating force transferred by the flexible wire 135 to a linear force for making the portion of the flexible wire placed inside of the member longer and shorter to thereby increase and decrease the diameter of the member 101 for stretching the intestinal wall to create an induced feeling of satiety.

FIG. 34G shows an embodiment of a member 101, which may be used as a first member 101a in the embodiments shown with reference to FIGS. 34A-34D. In the embodiment shown in FIG. 34G, the member 101 is operated by a flexible wire 135 in the form of a Bowden cable configured to transfer linear force for directly actuating the member 101 by increasing the length of the circumference of the member 101, and thereby the diameter of the member 101. The flexible wire 135 loops around in the member 101 which is comprised of a semi-rigid sheath comprising elevated and lowered portions, such that the length (circumference) of the member 101 can be altered even as fibrotic tissue overgrows the member 101. In the embodiment shown in FIG. 34E, the sheath is shaped like a bellows and acts as a bellows by expanding and contracting as the length of the flexible wire inside of the member 101 is varied.

FIG. 34H shows an embodiment of the member 101 similar to the embodiment shown in FIGS. 34F and 34G, the difference being that in the embodiment of FIG. 34H, the member 101 is hydraulically operable. The member comprises a hydraulic cylinder 143 placed inside of the member 101 for expanding and contracting the member 101 which increases and decreases the diameter of the member 101. The hydraulic cylinder 143 is connected to a hydraulic pump placed in a remote unite by means of a fluid conduit 109. Features of the remote unit and the controller are further described with reference to FIGS. 33-65FH.

FIG. 341 shows an embodiment of the member 101 similar to the embodiment shown in FIG. 34H, the difference being that in the embodiment of FIG. 341, the hydraulic cylinder (143) is replaced by an electrical actuator A, which may comprise an electrical motor, such as a piezoelectric motor, or a piezoelectric actuator, or a solenoid. The electrical actuator A is connected to a controller by means of an electrical lead 474. The controller may be placed in a remote unit. Features of the remote unit and the controller are further described with reference to FIGS. 33-65FH.

The medical device 10 in any of the embodiments shown with reference to FIGS. 34A—341, may further comprise a sensor configured to sense at least one of: a temperature locally at the members, a parameter related to strain in at least one of the first and second members, and a hydraulic pressure in at least one of the hydraulic actuator, the hydraulic pump, the hydraulic reservoir and the hydraulic conduit.

FIGS. 7-14D′, 28A—28C, 30A-31E, 31G-31I, 31M-31T, 31X-341 are examples of embodiments in which the medical device comprises an operable first member configured to be fixated to a first portion of the stomach wall, a second member configured to be fixated to a second portion of the stomach wall, a mechanical operation device configured to move at least a portion of the first member in a first direction away from at least one portion of the second member. A portion of the mechanical operation device is placed in a remote unit configured to be placed at a remote location in the body of the patient, the pre-implanted medical device further comprises a force transferring element configured to transfer mechanical force from the remote unit to the first member. Generally, a non-therapeutic method of creating a sensation of satiety using a pre-implanted medical device configured to stretch a portion of the stomach wall of a human is further provided. The non-therapeutic method generally comprises operating the portion of the mechanical operation device placed in the remote unit such that the force transferring element transfers force from the remote unit to the operable first member for stretching a portion of the stomach wall between the first portion of the stomach wall and the second portion of the stomach wall, for creating the sensation of satiety.

The non-therapeutic method of creating a feeling of satiety could be linked to a life-style choice of not eating too much or could be linked to a desired physical appearance and as such be linked to physical esthetics.

The medical devices of FIGS. 7-14D′, 28A—28C, 30A-31E, 31G-31I, 31M-31T, 31X-341 may comprise or be connected to an implantable controller configured to control the operation of the first member. In such embodiments, the non-therapeutic method comprises operating the first member as a result of the receipt of a wireless control signal at the controller, the wireless control signal being sent from a remote control external to the body of the patient, and/or the receipt of a sensor signal from a pre-implanted sensor connected to the controller, and/or the lapse of a pre-determined time which is programmed into the controller.

FIGS. 35A, 35B and 35C show an embodiment of a remote unit 140 which may be used in combination with any of the hydraulically operable medical devices disclosed herein. The remote unit 140 is configured to be held in position by a tissue portion 610 of a patient. The remote unit 140 comprises a first portion 141′ configured to be placed on a first side 612 of the tissue portion 610, the first portion 141′ having a first cross-sectional area A1 in a first plane P1 and comprising a first surface 614 configured to face a first tissue surface 616 of the first side 612 of the tissue portion 610. The remote unit 140 further comprises a second portion 141″ configured to be placed on a second side 618 of the tissue portion 610, the second side 618 opposing the first side 612, the second portion 141″ having a second cross-sectional area A2 in a second plane P2 and comprising a second surface 620 configured to engage a second tissue surface 622 of the second side 618 of the tissue portion 610. The remote unit 140 further comprises a connecting portion 142 configured to be placed through a hole in the tissue portion 610 extending between the first and second sides 612, 618 of the tissue portion 610. The connecting portion 142 here has a third cross-sectional area A3 in a third plane P3 and a fourth cross-sectional area A4 in a fourth plane P4 and a third surface 624 configured to engage the first tissue surface 616 of the first side 612 of the tissue portion 610. The connecting portion 142 is configured to connect the first portion 141′ to the second portion 141″.

The connecting portion 142 thus has a portion being sized and shaped to fit through the hole in the tissue portion 610, such portion having the third cross-sectional area A3. Furthermore, the connecting portion 142 may have another portion being sized and shaped to not fit through the hole in the tissue portion 610, such portion having the fourth cross-sectional area A4. Likewise, the second portion 141″ may have a portion being sized and shaped to not fit through the hole in the tissue portion 610, such portion having the second cross-sectional area A2. Thus, the connecting portion 142 may cooperate with the second portion 141″ to keep the device in place in the hole of the tissue portion 610.

In the embodiment illustrated in FIG. 35A, the first portion 141′ is configured to detachably connect, i.e. reversibly connect to the connecting portion 142 by a mechanical and/or magnetic mechanism. In the illustrated embodiment, a mechanic mechanism is used, wherein one or several spring-loaded spherical elements 601 lock in place in a groove 603 of the connecting portion 142 when the first portion 141′ is inserted into the connecting portion 142. Other locking mechanisms are envisioned, including corresponding threads and grooves, self-locking elements, and twist and lock fittings.

The remote unit 140 is configured such that, when implanted, the first portion 141′ will be placed closer to an outside of the patient than the second portion 141″. Furthermore, in some implantation procedures the remote unit 140 may be implanted such that space will be available beyond the second portion, i.e. beyond the second side 618 of the tissue portion 610, whereas there may be as much space on the first side 612 of the tissue portion. Furthermore, tissue and/or skin may exert a force on the first portion 141″ towards the tissue portion 610, and provide for that the second portion 141″ does not travel through the hole in the tissue portion towards the first side 612 of the tissue portion. Thus, it is preferably if the remote unit 140 is primarily configured to prevent the first portion 141″ from travelling through the hole in the tissue portion 612 towards the second side 618 of the tissue portion 610.

The first portion 141′ may further comprise one or several connections 605 for transferring energy and/or communication signals to the second portion 141″ via the connecting portion 142. The connections 605 in the illustrated embodiment are symmetrically arranged around a circumference of a protrusion 607 of the first portion 141′ and are arranged to engage with a corresponding connection 609 arranged at an inner surface of the connecting portion 142. The protrusion 607 may extend in a central extension C1 of the central portion 142. The second portion 141″ may also comprise one or several connections 611, which may be similarly arranged and configured as the connections 605 of the first portion 141′. For example, the one or several connections 611 may engage with the connection 609 of the connecting portion 142 to receive energy and/or communication signals from the first portion 141′. Although the protrusion 607 is illustrated separately in FIGS. 35A and 35B, it is to be understood that the protrusion 607 may be formed as one integral unit with the first portion 141′.

Other arrangements of connections are envisioned, such as asymmetrically arranged connections around the circumference of the protrusion 607. It is also envisioned that one or several connections may be arranged on the first surface 614 of the first portion 141′, wherein the connections are arranged to engage with corresponding connections arranged on the opposing surface 613 of the connecting portion. Such connections on the opposing surface 613 may cover a relatively large area as compared to the connection 609, thus allowing a larger area of contact and a higher rate and/or signal strength of energy and/or communication signal transfer. Furthermore, it is envisioned that a physical connection between the first portion 141′, connecting portion 142 and second portion 141″ may be replaced or accompanied by a wireless arrangement, as described further in other parts of the present disclosure.

Any of the first surface 614 of the first portion 141′, the second surface 620 of the second portion 141′, the third surface 624 of the connecting portion 142, and an opposing surface 613 of the connecting portion 142, may be provided with at least one of ribs, barbs, hooks, a friction enhancing surface treatment, and a friction enhancing material, to facilitate the remote unit 140 being held in position by the tissue portion, and/or to facilitate that the different parts of the device are held in mutual position.

The opposing surface 613 of the connecting portion 142 and the first surface 614 of the first portion 141′ may provide, fully or partly, a connection mechanism to detachably connect the first portion 141′ to the connecting portion 142. Such connection mechanisms have been described previously in the presented disclosure, and can be arranged on one or both of the opposing surface 613 and the first surface 614, and will not be further described here.

The opposing surface 613 may be provided with a recess configured to house at least part of the first portion 141′. In particular, such recess may be configured to receive at least a portion of the first portion 141′, including the first surface 614. Similarly, the first surface 614 may be provided with a recess configured to house at least part of the connecting portion 142. In particular, such recess may be configured to receive at least a portion of the connecting portion 142, and in some embodiments such recess may be configured to receive at least one protruding element to at least partially enclose at least one protruding element or flange.

In the illustrated embodiment, the first portion 141′ comprises a first energy storage unit 304a and a controller 300a comprising one or several processing units connected to the first energy storage unit 304a. The first energy storage unit 304a may be rechargeable by wireless transfer of energy. In some embodiments, the first energy storage unit 304a may be non-rechargeable. Upon reaching the life-time end of such first energy storage, a replacement first portion comprising a new first energy storage unit may simply be swapped in place for the first portion having the depleted first energy storage unit. The second portion 141″ may further comprise a controller 300b comprising one or several processing units. As will be described in other parts of the present disclosure, the first portion 141′ and the second portion 141″ may comprise one or several functional parts, such as receivers, transmitters, transceivers, control units, processing units, sensors, energy storage units, sensors, etc.

The remote unite 140 may be non-inflatable.

In FIGS. 35A and 35C, the second portion 141″ in the illustrated embodiment comprises a pump, the specific embodiment of which is disclosed with reference to FIG. 57h. However, it is to be understood that other embodiments of the second portion 141″ are able to be connected to the first portion 141′ via the connecting portion 142, such as second portions 141″ comprising a motor for providing mechanical work without the use of fluids or any of the other pumps described with reference to FIGS. 54a-58. Furthermore, although the connecting portion 142 is illustrated in FIG. 35A as a separate unit, the connecting portion 142 may form part of the second portion 141″ such that the portions forms part of a single unit.

The first portion 141′ may be detachably connected to at least one of the connecting portion 142 and the second portion 141″.

FIG. 35B shows an embodiment similar to that of the embodiment of FIG. 35A, the only difference being that the first and second hydraulic conduit 109a, 109b is connected to the hydraulic pump such that hydraulic fluid can be conducted to a first and second receiver of hydraulic fluid (e.g. for hydraulically operating a first and second member for stretching different portions of the stomach wall).

The outflow from the hydraulic pump to the conduits 109a, 109b is controlled by two valves V1, V2 which are electrically actuated and controlled from the controller 300 of the remote unit 140.

As can be seen in FIG. 35C, the first, second, third and fourth planes P1, P2, P3 and P4, are parallel to each other. Furthermore, in the illustrated embodiment, the third cross-sectional area A3 is smaller than the first, second and fourth cross-sectional areas A1, A2 and A4, such that the first portion 141′, second portion 141″ and connecting portion 142 are prevented from travelling through the hole in the tissue portion 610 in a direction perpendicular to the first, second and third planes P1, P2 and P3. Hereby, the second portion 141″ and the connecting portion 142 can be held in position by the tissue portion 610 of the patient also when the first portion 141′ is disconnected from the connecting portion 142.

It is to be understood that the illustrated planes P1, P2, P3 and P4 are merely an example of how such planes may intersect the remote unit 140. Other arrangements of planes are possible, as long as the conditions above are fulfilled, i.e. that the portions have cross-sectional areas, wherein the third cross-sectional area in the third plane P3 is smaller than the first, second and fourth cross-sectional areas, and that the planes P1, P2, P3 and P4 are parallel to each other.

The connecting portion 142 illustrated in FIG. 35A may be defined as a connecting portion 142 comprising a flange 626. The flange 626 thus comprises the fourth cross-sectional area A4 such that the flange 626 is prevented from travelling through the hole in the tissue portion 610 in a direction perpendicular to the first, second and third planes P1, P2 and P3. The flange 626 may protrude in a direction parallel to the first, second, third and fourth planes P1, P2, P3 and P4. This direction is perpendicular to a central extension C1 of the connecting portion 142.

The connecting portion 142 is not restricted to flanges, however. Other protruding elements may additionally or alternatively be incorporated into the connecting portion 142. As such, the connecting portion 142 may comprise at least one protruding element comprising the fourth cross-sectional area A4, such that the at least one protruding element is prevented from travelling through the hole in the tissue portion 610, such that the second portion 141″ and the connecting portion 142 can be held in position by the tissue portion 610 of the patient also when the first portion 141′ is disconnected from the connecting portion 142. The at least one protruding element may protrude in a direction parallel to the first, second, third and fourth planes P1, P2, P3 and P4. This direction is perpendicular to a central extension C1 of the connecting portion 142. As such, the at least one protruding element will also comprise the third surface configured to engage the first tissue surface 616 of the first side 612 of the tissue portion 610.

The connecting portion 142 may comprise a hollow portion 628. The hollow portion 628 may provide a passage between the first and second portions 141′, 141″. In particular, the hollow portion 628 may house a conduit for transferring fluid from the first portion 141′ to the second portion 141″. The hollow portion 628 may also comprise or house one or several connections or electrical leads for transferring energy and/or communication signals between the first portion 141′ and the second portion 141″.

Some relative dimensions of the remote unit 140 will now be described with reference to FIGS. 35C and 36A-36D, however it is to be understood that these dimensions may also apply to other embodiments of the remote unit 140. The at least one protruding element 626 may have a height HF in a direction perpendicular to the fourth plane being less than a height H1 of the first portion 141′ in said direction. The height HF may alternatively be less than half of said height H1 of the first portion 141′ in said direction, less than a quarter of said height H1 of the first portion 141′ in said direction, or less than a tenth of said height H1 of the first portion 141′ in said direction.

The height H1 of the first portion 141′ in a direction perpendicular to the first plane may be less than a height H2 of the second portion 141″ in said direction, such as less than half of said height H2 of the second portion 141″ in said direction, less than a quarter of said height H2 of the second portion 141″ in said direction, or less than a tenth of said height H2 of the second portion 141″ in said direction.

The at least one protruding element 626 may have a diameter DF in the fourth plane being one of less than a diameter D1 of the first portion 141′ in the first plane, equal to a diameter D1 of the first portion 141′ in the first plane, and larger than a diameter D1 of the first portion 141′ in the first plane. Similarly, the cross-sectional area of the at least one protruding element 626 in the fourth plane may be less, equal to, or larger than a cross-sectional area of the first portion in the first plane.

The at least one protruding element 626 may have a height HF in a direction perpendicular to the fourth plane being less than a height HC of the connecting portion 142 in said direction. Here, the height HC of the connecting portion 142 is defined as the height excluding the at least one protruding element, which forms part of the connecting portion 142. The height HF may alternatively be less than half of said height HC of the connecting portion 142 in said direction, less than a quarter of said height HC of the connecting portion 142 in said direction, or less than a tenth of said height HC of connecting portion 142 in said direction.

It is important to note that although the implantable energized medical device is disclosed herein as having a third cross-sectional area being smaller than a first cross-sectional area, this feature is not essential. The third cross-sectional area may be equal to or larger than the first cross-sectional area.

As shown in FIG. 36D, the first portion 141′ may have a first cross-sectional area A1 being equal to or smaller than the third cross-sectional area A3 of the connecting portion 142. In particular, the first portion 141′ does not necessarily need to provide a cross-sectional area being larger than the third cross-sectional area of connecting portion 142, intended to pass through a hole in the tissue, if the connecting portion 142 provides an additional cross-sectional area being larger than the third cross-sectional area of the connecting portion 142. The first portion 141′ as illustrated in FIG. 36D may comprise the components discussed elsewhere in the present disclosure, although not shown, such as an energy storage unit, receiver, transmitter, etc.

Wireless energy receivers and/or communication receivers and/or transmitters in the first portion 141′ may be configured to receive energy from and/or communicate wirelessly with an external device outside the body using electromagnetic waves at a frequency below 100 kHz, or more specifically below 40 kHz, or more specifically below 20 kHz. The wireless energy receivers and/or communication receivers and/or transmitters in the first portion 141′ may thus be configured to communicate with the external device using “Very Low Frequency” communication (VLF). VLF signals have the ability to penetrate a titanium housing of the implantable energized medical device, such that the electronics of the implantable medical device can be completely encapsulated in a titanium housing. In addition, or alternatively, communication and energy transfer between the first portion 141′ and second portion 141″ may be made using VLF signals. In such embodiments, receivers and transmitters (for energy and/or communication) of the first portion 141′ and second portion 141″ are configured accordingly.

As shown in FIGS. 37A-37B, the at least one protruding element 626 may have an annular shape, such as a disk shape. However, elliptical, elongated and/or other polyhedral or irregular shapes are also possible. In the illustrated embodiment, the at least one protruding element 626 extends a full revolution around the center axis of the connecting portion 142. However, other arrangements are possible, wherein the at least one protruding element 626 constitute a partial circle sector. In the case of a plurality of protruding elements, such plurality of protruding elements may constitute several partial circle sectors.

As shown in FIGS. 38A-38B, 39A-39B, the connecting portion 142 may comprise at least two protruding elements 626, 627. For example, the connecting portion 142 may comprise at least three, four, five, fix, seven, eight, nine, ten protruding elements, and so on. In such embodiments, the at least two protruding elements 626, 627 may together comprise the fourth cross-sectional area, thus providing a necessary cross-sectional area to prevent the first portion and second portion from travelling through the hole in the tissue portion.

The at least two protruding elements 626, 627 may be symmetrically arranged about the central axis of the connecting portion, as shown in FIGS. 38a-38b, or asymmetrically arranged about the central axis of the connecting portion, as shown in FIGS. 39a-39b. In particular, the at least two protruding elements 626, 627 may be asymmetrically arranged so as to be located towards one side of the connecting portion 142, as shown in FIGS. 39a-39b. The arrangement of protruding element(s) may allow the remote unit 140, and in particular the connecting portion 142, to be placed in areas of the patient where space is limited in one or more directions.

The first portion 141′ may comprise a first energy storage unit for supplying the remote unit 140 with energy.

Although one type or embodiment of the implantable remote unit 140, may fit most patients, it may be necessary to provide a selection of implantable remote units 140 or portions to be assembled into implantable remote units 140. For example, some patients may require different lengths, shapes, sizes, widths or heights depending on individual anatomy. Furthermore, some parts or portions of the implantable remote units 140 may be common among several different types or embodiments of remote units, while other parts or portions may be replaceable or interchangeable. Such parts or portions may include energy storage devices, communication devices, fluid connections, mechanical connections, electrical connections, and so on.

To provide flexibility and increase user friendliness, a kit of parts may be provided. The kit preferably comprises a group of one or more first portions, a group of one or more second portions, and a group of one or more connecting portions, the first portions, second portions and connecting portions being embodied as described throughout the present disclosure. At least one of the groups comprises at least two different types of said respective portions. By the term “type”, it is hereby meant a variety, class or embodiment of said respective portion.

In some embodiments of the kit, the group of one or more first portions, the group of one or more second portions, and the group of one or more connecting portions, comprise separate parts which may be assembled into a complete remote unit. The remote unit may thus be said to be modular, in that the first portion, the second portion, and/or the connecting portion may be interchanged for another type of the respective portion.

In some embodiments, the connecting portion form part of the first portion or the second portion.

With reference to FIG. 40, the kit for assembling the remote unit comprises a group 650 of one or more first portions 141′, in the illustrated example a group of one first portion 141′, a group 652 of one or more connecting portions 142, in the illustrated example a group of three connecting portions 142, and a group 654 of one or more second portions 141″, in the illustrated example a group of two second portions 141″. For simplicity, all types and combinations of first portions, second portions and connecting portions will not be illustrated or described in detail.

Accordingly, the group 652 of one or more connecting portions 142 comprise three different types of connecting portions 142. Here, the different types of connecting portions 142 comprise connecting portions 142a, 142b, 142c having different heights. Furthermore, the group 654 of one or more second portions 141″ comprise two different types of second portions 141″.

Here, the different types of second portions 141″ comprise a second portion 141″ a being configured to eccentrically connect to a connecting portion, having a first end and a second end as described in other parts of the present disclosure, wherein the second end of the second portion 141″ a comprises or is configured for at least one connection for connecting to an implant being located in a caudal direction from a location of the remote unit in the patient, when the device is assembled. In the illustrated figure, the at least one connection is visualized as a lead or wire. However, other embodiments are possible, including the second end comprising a port, connector or other type of connective element for transmission of power, fluid, and/or signals.

Furthermore, the different types of second portions 141″ comprise a second portion 141″b being configured to eccentrically connect to a connecting portion, having a first end and a second end as described in other parts of the present disclosure, wherein the first end of the second portion 141″b comprises or is configured for at least one connection for connecting to an implantable medical device for stretching the stomach wall of the patient, being located in a cranial direction from a location of the remote unit in the patient, when the device is assembled. In the illustrated figure, the at least one connection is visualized as a lead or wire. However, other embodiments are possible, including the first end comprising a port, connector or other type of connective element for transmission of power, fluid, and/or signals.

Thus, the remote unit may be modular, and different types of devices can be achieved by selecting and combining a first portion 141′, a connecting portion 142, and a second portion 141″, from each of the groups 652, 654, 656.

In the illustrated example, a first remote unit 140a is achieved by a selection of the first portion 141′, the connecting portion 142a, and the second portion 141″ a. Such remote unit 140a may be particularly advantageous in that the connecting portion 142a may be able to extend through a thick layer of tissue to connect the first portion 141′ and the second portion 141″ a. Another remote unit 140b is achieved by a selection of the first portion 141′, the connecting portion 142c, and the second portion 141″b. Such device may be particularly advantageous in that the connecting portion 142c has a smaller footprint than the connecting portion 142a, i.e. occupying less space in the patient. Owing to the modular property of the remote units 140a and 140b, a practician or surgeon may select a suitable connecting portion as needed upon having assessed the anatomy of a patient. Furthermore, since remote units 140a and 140b share a common type of first portions 141′, it will not be necessary for a practician or surgeon to maintain a stock of different first portions (or a stock of complete, assembled devices) merely for the sake of achieving a device having different connections located in the first end or second end of the second portion respectively, as in the case of second portions 141″ a, 141″b.

The example illustrated in FIG. 40 is merely exemplifying to display the idea of a modular implantable remote unit 140. The group 650 of one or more first portions 141′ may comprise a variety of different features, such as first portions with or without a first energy storage unit, with or without a first wireless energy receiver unit for receiving energy transmitted wirelessly by an external wireless energy transmitter, with or without an internal wireless energy transmitter, and/or other features as described throughout the present disclosure. Other features include different height, width, or length of the first portion. It is to be understood that first portions having one or more such features may be combined with a particular shape or dimensions to achieve a variety of first portions. The same applies to connecting portions and second portions.

With reference to FIG. 41, an embodiment of an implantable remote unit 140, will be described. The remote unit 140 is configured to be held in position by a tissue portion 610 of a patient.

The remote unit 140 comprises a first portion 141′ configured to be placed on a first side of the tissue portion 610, the first portion 141′ having a first cross-sectional area in a first plane and comprising a first surface configured to face and/or engage a first tissue surface of the first side of the tissue portion 610.

The device 140 further comprises a second portion 141″ configured to be placed on a second side of the tissue portion 610, the second side opposing the first side, the second portion 141″ having a second cross-sectional area in a second plane and comprising a second surface configured to engage a second tissue surface of the second side of the tissue portion 610. The remote unit 140 further comprises a connecting portion 142 configured to be placed through a hole in the tissue portion 610 extending between the first and second sides of the tissue portion 610. The connecting portion 142 here has a third cross-sectional area in a third plane. The connecting portion 142 is configured to connect the first portion 141′ to the second portion 141″. Here, the first portion 141′ comprises a first wireless energy receiver 308a for receiving energy transmitted wirelessly by an external wireless energy transmitter, and an internal wireless energy transmitter 308a configured to transmit energy wirelessly to the second portion. Furthermore, the second portion here comprises a second wireless energy receiver 308b configured to receive energy transmitted wirelessly by the internal wireless energy transmitter 308a.

Although receivers and transmitters may be discussed and illustrated separately in the present disclosure, it is to be understood that the receivers and/or transmitters may be comprised in a transceiver. Furthermore, the receivers and/or transmitters in the first portion 141′ and second portion 141″ respectively may form part of a single receiving or transmitting unit configured for receiving or transmitting energy and/or communication signals, including data. Furthermore, the internal wireless energy transmitter and/or a first wireless communication receiver/transmitter may be a separate unit 308c located in a lower portion of the first portion 141′, referred to as a proximal end of the first portion 141′ in other parts of the present disclosure, close to the connecting portion 142 and the second portion 141″. Such placement may provide for that energy and/or communication signals transmitted by the unit 308c will not be attenuated by internal components of the first portion 141′ when being transmitted to the second portion 141″. Such internal components may include a first energy storage unit 304a.

The first portion 141′ here comprises a first energy storage unit 304a connected to the first wireless energy receiver 308a. The second portion comprises a second energy storage unit 304b connected to the second wireless energy receiver 308b. Such an energy storage unit may be a solid-state battery, such as a thionyl-chloride battery.

In some embodiments, the first wireless energy receiver 308a is configured to receive energy transmitted wirelessly by the external wireless energy transmitter and store the received energy in the first energy storage unit 304a. Furthermore, the internal wireless energy transmitter 308a is configured to wirelessly transmit energy stored in the first energy storage unit 304a to the second wireless energy receiver 308b, and the second wireless energy receiver 308b is configured to receive energy transmitted wirelessly by the internal wireless energy transmitter 308a and store the received energy in the second energy storage unit 305b.

The first energy storage unit 304a may be configured to store less energy than the second energy storage unit 304b, and/or configured to be charged faster than the second energy storage unit 304b. Hereby, charging of the first energy storage unit 304a may be relatively quick, whereas transfer of energy from the first energy storage unit 304a to the second energy storage unit 304b may be relatively slow. Thus, a user can quickly charge the first energy storage unit 304a, and will not during such charging be restricted for a long period of time by being connected to an external wireless energy transmitter, e.g. at a particular location. After having charged the first energy storage unit 304a, the user may move freely while energy slowly transfers from the first energy storage unit 304a to the second energy storage unit 304b, via the first wireless energy transmitter 308a,c and the second wireless energy receiver 308b.

The first portion may comprise a first controller comprising at least one processing unit 306a. The second portion may comprise a second controller comprising at least one processing unit 306b.

At least one of the first and second processing unit 306a, 306b may be connected to a wireless transceiver 308a,b,c for communicating wirelessly with an external device.

The first controller may be connected to a first wireless communication receiver 308a,c in the first portion 141′ for receiving wireless communication from an external device and/or from a wireless communication transmitter 308b in the second portion 141″. Furthermore, the first controller may be connected to a first wireless communication transmitter 308a,c in the first portion 141′ for transmitting wireless communication to a second wireless communication receiver 308b in the second portion 141″. The second controller may be connected to the second wireless communication receiver 308b for receiving wireless communication from the first portion 141′. The second controller may further be connected to a second wireless communication transmitter 308b for transmitting wireless communication to the first portion 141′.

In some embodiments, the first wireless energy receiver 308a comprises a first coil, and the wireless energy transmitter 308a,c comprises a second coil, as shown in FIG. 52.

The device may further comprise at least one sensor (not shown) for providing input to at least one of the first and second controller. Such sensor data may be transmitted to an external device via the first wireless communication transmitter 308a and/or the second wireless communication transmitter 308b. The sensor may be or comprise a sensor configured to sense a physical parameter of the device 140. The sensor may also be or comprise a sensor configured to sense at least one of a temperature of the remote unit 140, a temperature of an implantable device for stretching the stomach wall (which may be located in the main portion), a parameter related to the power consumption of the device, a parameter related to the power consumption of an implantable device for stretching the stomach wall, a parameter related to a status of at least one of the first and second energy storage unit 304a, 304b, a parameter related to the wireless transfer of energy from a source external to the body of the patient, and a hydraulic pressure. The sensor may also be or comprise a sensor configured to sense a physiological parameter of the patient, such as at least one of a parameter related to the patient swallowing, a local temperature, a systemic temperature, a blood saturation, a blood oxygenation, a blood pressure, a parameter related to an ischemia marker, or pH. The sensor configured to sense a parameter related to the patient swallowing may comprise at least one of a motility sensor, a acoustic sensor, an optical sensor, and a strain sensor. The sensor configured to sense pH may be configured to sense the acidity in the stomach.

The sensor may be configured to sense a temperature of the device 140, to avoid excessive heating of tissue connected to the device during operation of the device, or during operation of an external implant using the device, or charging of an energy storage unit in the device 140. Excessive heating may also damage the device and/or the energy storage unit. Excessive heating may also be an indicator that something is wrong with the device and may be used for triggering an alarm function for alerting the patient or physician. The sensor may also be configured to sense a parameter related to the power consumption of the device 140 or the power consumption of an external implant being powered by the device 140, to avoid excessive power consumption which may drain and/or damage the energy storage unit of the device 140. Excessive power consumption may also be an indicator that something is wrong with the device 140 and may be used for triggering an alarm function for alerting the patient or physician.

With reference to FIGS. 42, 45A and 45B, an embodiment of an implantable remote unit 140 will be described. The remote unit 140 is configured to be held in position by a tissue portion 610 of a patient. The remote unit 140 comprises a first portion 141′ configured to be placed on a first side 612 of the tissue portion 610, the first portion 141′ having a first cross-sectional area A1 in a first plane P1 and comprising a first surface 614 configured to face and/or engage a first tissue surface 616 of the first side 612 of the tissue portion 610. The remote unit 140 further comprises a second portion 141″ configured to be placed on a second side 618 of the tissue portion 610, the second side 618 opposing the first side 612, the second portion 141″ having a second cross-sectional area A2 in a second plane P2 and comprising a second surface 620 configured to engage a second tissue surface 622 of the second side 618 of the tissue portion 610. The remote unit 140 further comprises a connecting portion 142 configured to be placed through a hole in the tissue portion 610 extending between the first and second sides 612, 618 of the tissue portion 610. The connecting portion 142 here has a third cross-sectional area A3 in a third plane P3. The connecting portion 142 is configured to connect the first portion 141′ to the second portion 141″. In the illustrated embodiment, a connecting interface 630 between the connecting portion 142 and the second portion 141″ is eccentric with respect to the second portion 141″.

The first portion 141′ has an elongated shape in the illustrated embodiment of FIG. 42. Similarly, the second portion 141″ has an elongated shape. However, the first portion 141′ and/or second portion 141″ may assume other shapes, such as a flat disk e.g. having a width and length being larger than the height, a sphere, an ellipsoid, or any other polyhedral or irregular shape, some of these being exemplified in FIGS. 42-44.

As illustrated in FIGS. 45A and 45B, the connecting interface 630 between the connecting portion 142 and the second portion 141″ may be eccentric, with respect to the second portion 141″ in a first direction 631, but not in a second direction 633 being perpendicular to the first direction. The first direction 631 is here parallel to the line A-A, to the second plane P2, and to a length of the second portion 141″. The second direction 633 is here parallel to the line B-B, to the second plane P2, and to a width of the second portion 141″. It is also possible that the connecting interface between the connecting portion 142 and the second portion 141″ is eccentric, with respect to the second portion 141″, in the first direction 631 as well as in the second direction 633 being perpendicular to the first direction 631.

Similarly, a connecting interface between the connecting portion 142 and the first portion 141′ may be eccentric with respect to the first portion 141′ in the first direction 631, and/or in the second direction 633.

The first portion 141′, connecting portion 142 and second portion 141″ may structurally form one integral unit. It is however also possible that the first portion 141′ and the connecting portion 142 structurally form one integral unit, while the second portion 141″ form a separate unit, or, that the second portion 141″ and the connecting portion 142 structurally form one integral unit, while the first portion 141′ form a separate unit.

Additionally, or alternatively, the second portion 141″ may comprise a removable and/or interchangeable portion 639. In some embodiments, the removable portion 639 may form part of a distal region which will be further described in other parts of the present disclosure. A removable portion may also form part of a proximal region. Thus, the second portion 141″ may comprise at least two removable portions, each being arranged at a respective end of the second portion 141″. The removable portion 639 may house, hold or comprise one or several functional parts of the remote unit 140, such as gears, motors, connections, reservoirs, and the like as described in other parts of the present disclosure. An embodiment having such removable portion 639 will be able to be modified as necessary to circumstances of a particular patient.

In the case of the first portion 141′, connecting portion 142 and second portion 141″ structurally forming one integral unit, the eccentric connecting interface between the connecting portion 142 and the second portion 141″, with respect to the second portion 141″, will provide for that the remote unit 140 will be able to be inserted into the hole in the tissue portion. The remote unit 140 may for example be inserted into the hole at an angle, similar to how a foot is inserted into a shoe, to allow most or all of the second portion 141″ to pass through the hole, before it is angled, rotated, and/or pivoted to allow any remaining portion of the second portion 141″ to pass through the hole and allow the remote unit 140 to assume its intended position.

As illustrated in FIGS. 42—44, the first portion 141′ may assume a variety of shapes, such as an oblong shape, a flat disk shape, a spherical shape, or any other polyhedral or irregular shape. Similarly, the second portion 141″ may assume a variety of shapes, such as an oblong shape, a flat disk shape, a spherical shape, or any other polyhedral or irregular shape. The proposed shapes of the first and second portions 141′, 141″ may be mixed and combined to form embodiments not exemplified in the illustrated embodiments. For example, one or both of the first and second portions 141′, 141″ may have a flat oblong shape. In this context, the term “flat” is related to the height of the first or second portion 141′, 141″, i.e. in a direction parallel to a central extension C1 of the connecting portion 142. The term “oblong” is related to a length of the first or second portion 141′, 141″. A definition of such length is further discussed in other parts of the present disclosure.

With reference to FIGS. 45A-45B, the second portion 141″ has a first end 632 and a second end 634 opposing the first end 632. The length of the second portion 141″ is defined as the length between the first end 632 and the second end 634. The length of the second portion 141″ is furthermore extending in a direction being different to the central extension C1 of the connecting portion 142. The first end 632 and second end 634 are separated in a direction parallel to the second plane P2. Similarly, the first portion 141′ has a length between a first and a second end, the length extending in a direction being different to the central extension C1 of the connecting portion 142.

The second portion 141″ may be curved along its length. For example, one or both ends of the second portion 141″ may point in a direction being substantially different from the second plane P2, i.e. curving away from or towards the tissue portion when implanted. In some embodiments, the second portion 141″ curves within the second plane P2, exclusively or in combination with curving in other planes. The second portion 141″ may also be curved in more than one direction, i.e. along its length and along its width, the width extending in a direction perpendicular to the length.

The first and second ends 632, 634 of the second portion 141″ may comprise an elliptical point respectively. For example, the first and second ends 632, 634 may comprise a hemispherical end cap respectively. It is to be understood that also the first and second ends of the first portion 141′ may have such features.

The second portion 141″ may have at least one circular cross-section along the length between the first end 632 and second end 634, as illustrated in FIG. 42. It is however possible for the second portion 141″ to have at least one oval cross-section or at least one elliptical cross-section along the length between the first end 632 and the second end 634. Such cross-sectional shapes may also exist between ends in a width direction of the second portion 141″. Similarly, such cross-sectional shapes may also exist between ends in a length and/or width direction in the first portion 141′.

In the following paragraphs, some features and properties of the second portion 141″ will be described. It is however to be understood that these features and properties may also apply to the first portion 141′.

The second portion 141″ has a proximal region 636, an intermediate region 638, and a distal region 640. The proximal region 636 extends from the first end 632 to an interface between the connecting portion 142 and the second portion 141″, the intermediate region 638 is defined by the connecting interface 630 between the connecting portion 142 and the second portion 141″, and the distal region 640 extends from the connecting interface 630 between the connecting portion 142 and the second portion 141″ to the second end 634. The proximal region 636 is shorter than the distal region 640 with respect to the length of the second portion, i.e. with respect to the length direction 631. Thus, a heel (the proximal region) and a toe (the distal region) is present in the second portion 141″.

The second surface 620, configured to engage with the second tissue surface 622 of the second side 618 of the tissue portion 610, is part of the proximal region 636 and the distal region 640. If a length of the second portion 141″ is defined as x, and the width of the second portion 141″ is defined as y along respective length and width directions 631, 633 being perpendicular to each other and substantially parallel to the second plane P2, the connecting interface between the connecting portion 142 and the second portion 141″ is contained within a region extending from x>0 to x<x/2 and/or y>0 to y<y/2, x and y and 0 being respective end points of the second portion 141″ along said length and width directions. In other words, the connecting interface between the connecting portion 142 and the second portion 141″ is eccentric in at least one direction with respect to the second portion 141″, such that a heel and atoe is formed in the second portion 141″.

The first surface 614 configured to face and/or engage the first tissue surface 616 of the first side 612 of the tissue portion 610 may be substantially flat. In other words, the first portion 141′ may comprise a substantially flat side facing towards the tissue portion 610. Furthermore, an opposing surface of the first portion 141′, facing away from the tissue portion 610, may be substantially flat. Similarly, the second surface 620 configured to engage the second tissue surface 622 of the second side 618 of the tissue portion 610 may be substantially flat. In other words, the second portion 141″ may comprise a substantially flat side facing towards the tissue portion 610. Furthermore, an opposing surface of the second portion 141″, facing away from the tissue portion 610, may be substantially flat.

The second portion 141″ may be tapered from the first end 632 to the second end 634, thus giving the second portion 141″ different heights and/or widths along the length of the second portion 141″. The second portion may also be tapered from each of the first end 632 and second end 634 towards the intermediate region 638 of the second portion 141″.

Some dimensions of the first portion 141′, the second portion 141″ and the connecting portion 142 will now be disclosed. Any of the following disclosures of numerical intervals may include or exclude the end points of said intervals.

The first portion 141′ may have a maximum dimension being in the range of 10 to 60 mm, such as in the range of 10 to 40 mm such as in the range of 10 to 30 mm, such as in the range of 10 to 25 mm, such as in the range of 15 to 40 mm, such as in the range of 15 to 35 mm, such as in the range of 15 to 30 mm, such as in the range of 15 to 25 mm. By the term “maximum dimension” it is hereby meant the largest dimension in any direction.

The first portion 141′ may have a diameter being in the range of 10 to 60 mm, such as in the range of 10 to 40 mm such as in the range of 10 to 30 mm, such as in the range of 10 to 25 mm, such as in the range of 15 to 40 mm, such as in the range of 15 to 35 mm, such as in the range of 15 to 30 mm, such as in the range of 15 to 25 mm.

The connecting portion 142 may have a maximum dimension in the third plane P3 in the range of 2 to 20 mm, such as in the range of 2 to 15 mm, such as in the range of 2 to 10 mm, such as in the range of 5 to 10 mm, such as in the range of 8 to 20 mm, such as in the range of 8 to 15 mm, such as in the range of 8 to 10 mm.

The second portion 141″ may have a maximum dimension being in the range of 30 to 90 mm, such as in the range of 30 to 70 mm, such as in the range of 30 to 60 mm, such as in the range of 30 to 40 mm, such as in the range of 35 to 90 mm, such as in the range of 35 to 70 mm, such as in the range of 35 to 60 mm, such as in the range of 35 to 40 mm.

The first portion has a first height H1, and the second portion has a second height H2, both heights being in a direction perpendicular to the first and second planes P1, P2. The first height may be smaller than the second height. However, in the embodiments illustrated in FIGS. 45A-45B, the first height H1 is substantially equal to the second height H2. Other height ratios are possible, for example the first height Hi may be less than ⅔ of the second height H2, such as less than ½ of the second height H2, such as less than ⅓ of the second height H2, such as less than ¼ of the second height H2, such as less than ⅕ of the second height H2, such as less than 1/10 of the second height H2.

As illustrated in FIGS. 45A-45B, the proximal region 636 has a length 642 being shorter than a length 646 of the distal region 640. The intermediate region 638 has a length 644, and a width 648.

In some embodiments, the length 644 of the intermediate region 638 is longer than the width 648. In other words, the connecting interface between the connecting portion 142 and the second portion 141″ may be elongated, having a longer dimension (in the exemplified case, the length) and a shorter dimension (in the exemplified case, the width). It is also possible that the length 644 of the intermediate region 638 is shorter than the width 648 of the intermediate region 638.

The length 646 of the distal region 640 is preferably longer than the length 644 of the intermediate region 638, however, an equally long distal region 640 and intermediate region 638, or a shorter distal region 640 than the intermediate region 638, is also possible. The length 642 of the proximal region 636 may be shorter than, equal to, or longer than the length 644 of the intermediate region 638.

The length 644 of the intermediate region 638 is preferably less than half of the length of the second portion 141″, i.e. less than half of the combined length of the proximal region 636, the intermediate region 638, and the distal region 630. In some embodiments, the length 644 of the intermediate region 638 is less than a third of the length of the second portion 141″, such as less than a fourth, less than a fifth, or less than a tenth of the length of the second portion 141″.

The connecting portion may have one of an oval cross-section, an elongated cross-section, and a circular cross-section, in a plane parallel to the third plane P3. In particular, the connecting portion may have several different cross-sectional shapes along its length in the central extension C1.

FIGS. 45C-45D illustrate an embodiment similar to the one described in conjunction with FIGS. 45a-45b. However, the embodiment of FIGS. 45c-45d lacks a proximal portion, i.e. the second portion 141″ does not comprise a “heel”. Furthermore, such embodiment may have a connecting portion 142 having a length and width, in directions 631 and 633 respectively, being equal to a height of the second portion in a direction parallel to the central extension C1, as illustrated. Thus, the connecting portion 142 and the second portion 141″ may be constituted by a substantially uniformly wide body.

In some embodiments the distal region 640 is configured to be directed downwards in a standing patient, i.e. in a caudal direction when the remote unit 140 is implanted. As illustrated in FIGS. 46A-46D, different orientations of the second portion 141″ relative the first portion 141′ are possible.

In some embodiments, a connection between either the first portion 141′ and the connecting portion 142, or between the second portion 141″ and the connecting portion 142, may allow for a plurality of different connecting orientations. For example, a connection mechanism between the first portion 141′ and the connecting portion 142 (or between the second portion 141″ and the connecting portion 142) may possess a 90 degree rotational symmetry to allow the second portion 141′ to be set in four different positions with respect to the first portion 141, each differing from the other by 90 degrees. Other degrees of rotational symmetry are of course possible, such as 30 degrees, 45 degrees, 60 degrees, 120 degrees, 180 degrees and so on. In other embodiments there are no connective mechanism between any of the first portion 141′, the connecting portion 142, and the second portion 141″ (i.e. the portions are made as one integral unit), and in such cases different variants of the device 140 can be achieved during manufacturing. In other embodiments, the connective mechanism between the first portion 141′ and the connecting portion 142 (or between the second portion 141″ and the connecting portion 142) is non-reversible, i.e. the first portion 141′ and the second portion 141″ may initially be handled as separate parts, but the orientation of the second portion 141″ relative the first portion 141′ cannot be changed once it has been selected and the parts have been connected via the connecting portion 142.

The different orientations of the second portion 141″ relative the first portion 141′ may be defined as the length direction of the second portion 141″ having a relation or angle with respect to a length direction of the first portion 141′. Such angle may be 15 degrees, 30, 45, 60, 75 90, 105, 120, 135, 150, 165, 180, 195, 210, 225, 240, 255, 270, 285, 300, 315, 330, 345 or 360 degrees. In particular, the angle between the first portion 141′ and the second portion 141″ may be defined as an angle in the planes P1 and P2, or as an angle in a plane parallel to the tissue portion 610, when the remote unit 140 is implanted. In the embodiment illustrated in FIGS. 46A-46D, the length direction of the second portion 141″ is angled by 0, 90, 180, and 270 degrees with respect to the length direction of the first portion 141′.

Referring now to FIGS. 46e-k, 46m, 46n, 46p and 46q. The following will discuss some features of the first portion 141′, and in some cases additionally or alternatively of the connecting portion 142, which enable the first portion 141′ to increase its cross-sectional area in the first plane (i.e. to increase an area of the first surface configured to face the first tissue surface), and/or which enable the first portion 141′ to be rotated, translated, or otherwise moved in relation to the connecting portion 142.

In some embodiments, the first portion 141′ will be configured to extend further away from the connecting portion 142 in or within the first plane. It is to be understood that these features can be combined with other features of the implantable energized medical device. In particular, the specific shape of the first portion, connecting portion and/or second portion in the illustrated embodiments are merely exemplary. Other shapes are possible, as discussed in the present disclosure. Accordingly, the elongated second portion 141″ does not necessarily need to be elongated as shown for example in FIG. 46e, and furthermore, the first portion 141′ does not necessarily need to have a semicircular shape.

With reference to FIG. 46e, an implantable energized medical device 140 is shown, wherein the first portion 141′ is configured and shaped such that an edge 710 of the first portion 141′ is substantially aligned with the connecting portion 142 with regard to the first direction 631. In other words, no part of the first portion 141′ protrudes forward of the connecting portion 142 with regard to the first direction 631. Hereby, insertion of the implantable energized medical device 140 may be facilitated, in particular when angled downwards, since the first portion 141′ will not abut the tissue until most or all of the second portion 141″ has been inserted through the hole in the tissue. Although the edge 710, as well as other edges of the first portion 141′, are hereby shown as having no radius, radiused edges are possible. Thus, the edge 710 may have a radius, and/or the first portion 141′, and/or the second portion 141″, and/or the connecting portion 142, may comprise radiused edges.

With reference to FIGS. 46f and 46g, a first portion 141′ is shown being configured to have its surface area increased. Here, the first cross-sectional area is increased, thereby increasing an area of the first surface configured to face (and in some embodiments also configured to contact) the first tissue surface. In the illustrated embodiment, the first portion 141′ comprises a first element 712 and a second element 714 being hingedly interconnected to allow the first element 712 to assume a first state (not shown) wherein the first element 712 is arranged on top of the second element 714, and a second state wherein the first element 712 is folded to be located adjacent or next to the second element 714. A similar configuration may be achieved by other means of interconnection between the first element 712 and second element 714, i.e. the configuration is not limited to a hinge-type connection. For example, the first element 712 and second element 714 may be constructed of a single piece of material being flexible enough to be able to fold over itself to assume the first and second state respectively.

Preferably, the first and second element 712, 714 are interconnected and formed such that a transition between the first and second element 712, 714 along the first direction 631 is flush. Furthermore, while in the first state, the first portion 141′ may possess the same feature as discussed in conjunction with FIG. 46e, i.e. the first portion 141′ may be substantially aligned with the connecting portion 142.

With reference to FIGS. 46h and 46i, a first portion 141′ is shown being configured to have its surface area increased. Here, the first cross-sectional area is increased, thereby increasing an area of the first surface configured to face (and in some embodiments also configured to contact) the first tissue surface. In the illustrated embodiment, the first portion 141′ comprises a first element 712 and a second element 714. The second element 714 here comprises a slot 715 configured to partially or fully house the first element 712. The first element 712 is configured to rotate about an axis to assume a first state, wherein the first element 712 is partially or completely housed in within the slot 715, and a second state wherein the first element 712 protrudes from the slot 715 to increase the first cross-sectional area. The first element 712 may be configured to rotate 180 degrees about the axis. In the illustrated example, the first and second elements 712, 714 are shaped as semi-circles and form a shape conforming to a full circle in the second state. However, it is also possible that the first element 712 only rotate about the axis up to 90 degrees, thus forming a shape conforming to three quarters of a circle in the second state. Other shapes are also possible, e.g. polygons.

With reference to FIGS. 46j and 46k, a similar configuration as described with reference to FIGS. 46h and 46i is shown. However, here the second element 714 does not comprise a slot, and the first element is thus not housed in a slot. Instead, the first element 712 is arranged on top of the second element 714 (similar to the embodiment of FIGS. 46f and 46g). The first portion 141′ is here configured to have its surface area increased, in particular the first cross-sectional area is increased, thereby increasing an area of the first surface configured to face (and in some embodiments also configured to contact) the first tissue surface. The first element 712 is configured to rotate about an axis to assume a first state, wherein the first element 712 is partially or completely arranged on top of the second element 714. Here, “completely arranged on top of” means that the first element 712 is confined within the borders of the second element 714. By rotation of the first element 712 about the axis, the first element 712 can assume a second state wherein the first element 712 protrudes over an edge or border of the second element 714 to increase the first cross-sectional area. The first element 712 may be configured to rotate 180 degrees about the axis. However, it is also possible that the first element 712 only rotate about the axis up to 90 degrees. Other shapes of the first and second element 712, 714 are also possible, e.g. polygons.

With reference to FIGS. 46m and 46n, a first portion 141′ is shown being configured to have its surface area increased. Here, the first cross-sectional area is increased, thereby increasing an area of the first surface configured to face (and in some embodiments also configured to contact) the first tissue surface. In the illustrated embodiment, the first portion 141′ comprises a first element 712 and a second element 714. The first element 712 here comprises a slot configured to partially or completely house the second element 714. The first element 712 is configured to assume a first state, as shown in FIG. 46m, wherein the second element 714 is arranged partially or fully within the slot of the first element 712, and a second state, as shown in FIG. 46n, wherein the first element 712 has been moved in a first direction to cause the second element 714 to protrude from the slot of the first element 712, and to cause the first element 712 to extend further away from the connecting portion 142 in the first plane. As will be understood, other variations are possible, e.g. the second element 714 may comprise the slot, and the first element 712 may be partially or fully housed within such slot, and subsequently the first element 712 or the second element 714 may be moved to protrude from such slot.

With reference to FIGS. 46p and 46q, a first portion 141′ is shown being configured to be moved in relation to the connecting portion 142. The expression “configured to be moved” may in this context be interpreted as the first portion 141′ being configured to assume at least two different positions with regard to the connecting portion 142 while still remaining in direct contact with the connecting portion. Here, the connecting portion 142 comprises a protruding element 717 and the first portion 141′ comprises a slot 718, wherein the protruding element 717 is configured to slide within the slot 718 along a predetermined path, e.g. in a first direction and a direction opposite said first direction. The protruding element 717 may be configured to be interlocked within the slot 718 such that the protruding element 717 can only be removed from the slot 718 in a preconfigured position. In other embodiments, the protruding element 717 may be permanently enclosed within the slot 718. By sliding the first portion 141′ in the first direction, an extension of the first portion 141′ in the first plane with respect to the connecting portion 142 will be able to be adjusted. Any position between the endpoints of the slot 718 may be able to be assumed by the first portion 141′. In particular, first portion 141′ and/or the connecting portion 142 may comprise a locking mechanism configured to secure a position of the first portion 141′ in relation to the connecting portion 142. Such locking mechanism may rely on flexible parts being biased towards each other to maintain the first portion 141′ and connecting portion 142 in a fixed position in relation to each other. Other possible locking mechanisms include the use of friction, snap-locking means, etc.

The second end 634 of the second portion 141″ may comprise one or several connections for connecting to an implant being located in a caudal direction from a location of the remote unit in the patient. Hereby, when the remote unit 140 is implanted in a patient, preferably with the distal region 640 and second end 634 pointing downwards in a standing patient, the connections will be closer to the implant as the second end 634 will be pointing in the caudal direction whereas the first end 632 will be pointing in the cranial direction. It is also possible that the second end 634 of the second portion 141″ is configured for connecting to an implant, i.e. the second end 634 may comprise a port, connector or other type of connective element for transmission of power, fluid, and/or signals.

Likewise, the first end 632 of the second portion 141″ may comprise one or several connections for connecting to an implant being located in a cranial direction from a location of the remote unit in the patient. Hereby, when the remote unit 140 is implanted in a patient, preferably with the distal region 640 and second end 634 pointing downwards in a standing patient, the connections will be closer to the implant as the first end 632 will be pointing in the cranial direction whereas the second end 634 will be pointing in the caudal direction. It is also possible that the first end 632 of the second portion 141″ is configured for connecting to an implant, i.e. the first end 632 may comprise a port, connector or other type of connective element for transmission of power, fluid, and/or signals.

With reference to FIGS. 47 and 48, an embodiment of an implantable remote unit 140 will be described. The remote unite 140 is configured to be held in position by a tissue portion 610 of a patient.

The remote unit 140 comprises a first portion 141′ configured to be placed on a first side 612 of the tissue portion 610, the first portion 141′ having a first cross-sectional area in a first plane and comprising a first surface 614 configured to face and/or engage a first tissue surface 616 of the first side 612 of the tissue portion 610. The remote unit 140 further comprises a second portion 141″ configured to be placed on a second side 618 of the tissue portion 610, the second side 618 opposing the first side 612, the second portion 141″ having a second cross-sectional area in a second plane and comprising a second surface 620 configured to engage a second tissue surface 622 of the second side 618 of the tissue portion 610. The remote unit 140 further comprises a connecting portion 142 configured to be placed through a hole in the tissue portion 610 extending between the first and second sides 612, 618 of the tissue portion 610. The connecting portion 142 here has a third cross-sectional area in a third plane. The connecting portion 142 is configured to connect the first portion 141′ to the second portion 141″.

With reference to FIG. 49, the first cross-sectional area has a first cross-sectional distance CD1a and a second cross-sectional distance CD2a, the first and second cross-sectional distances CD1a, CD2a being perpendicular to each other and the first cross-sectional distance CD1a being longer than the second cross-sectional distance CD2a. Furthermore, the second cross-sectional area has a first cross-sectional distance CD1b and a second cross-sectional distance CD2b, the first and second cross-sectional distances CD2a, CD2b being perpendicular to each other and the first cross-sectional distance CD1b being longer than the second cross-sectional distance CD2b. The first cross-sectional distance CD1a of the first cross-sectional area and the first cross-sectional distance CD1b of the second cross-sectional area are rotationally displaced in relation to each other with an angle exceeding 45° to facilitate insertion of the second portion 141″ through the hole in the tissue portion. In the embodiment illustrated in FIG. 49, the rotational displacement is 90°.

The rotational displacement of the first portion 141′ and the second portion 141″ forms a cross-like structure, being particularly advantageous in that insertion through the hole in the tissue portion 610 may be facilitated, and once positioned in the hole in the tissue portion 610 a secure position may be achieved. In particular, if the remote unit 140 is positioned such that the second portion 141″ has its first cross-sectional distance CD1b extending along a length extension of the hole 611 in the tissue portion 610, insertion of the second portion 141″ through the hole 611 may be facilitated. Furthermore, if the first portion 141′ is then displaced in relation to the second portion 141″ such that the first cross-sectional distance CD 1a of the first portion 141′ is displaced in relation to a length extension of the hole 611, the first portion 141′ may be prevented from travelling through the hole 611 in the tissue portion. In these cases, it is particularly advantageous if the hole 611 in the tissue portion is oblong, ellipsoidal, or at least has one dimension in one direction being longer than a dimension in another direction. Such oblong holes in a tissue portion may be formed for example in tissue having a fiber direction, where the longest dimension of the hole may be aligned with the fiber direction.

In the embodiment illustrated in FIG. 47, the first surface 614 of the first portion 141′ is flat, thus providing a larger contact surface to the first tissue surface 616 and consequently less pressure on the tissue portion. A more stable position may also be achieved by the flat surface. Also the second surface 620 of the second portion 141″ may be flat. However, other shapes, such as those described in other parts of the present disclosure, are possible.

As shown in FIG. 49, the connecting portion 142 may have an elongated cross-section in the third plane. It may be particularly advantageous if the connecting portion 142 has a longer length 644 than width 648, said length 644 extending in the same direction as a length direction of the second portion 141″, i.e. in the same direction as an elongation of the second portion 141″. Hereby, the elongation of the connecting portion 142 may run in the same direction as an elongation of the hole in the tissue portion.

With reference to FIG. 50, the rotational displacement of first cross-sectional distance of the first cross-sectional area and the first cross-sectional distance of the second cross-sectional area is shown, here at an angle about 45°. Accordingly, there is a rotational displacement, in the first, second and third planes, between a length direction 633 of the first portion 141′ and a length direction 631 of the second portion 141″. Other angles of rotational displacement are possible, such as 60°, 75, 90°, 105°, 120°, 135°, etc.

One and the same remote unit 140 may be capable of assuming several different arrangements with regards to rotational displacement of the first portion 141′ and the second portion 141″. In particular, this is possible when the first portion 141′ and/or the second portion 141″ is configured to detachably connect to the interconnecting portion 142. For example, a connection mechanism between the first portion 141′ and the connecting portion 142, or between the second portion 141″ and the connecting portion 142, may possess a rotational symmetry to allow the first portion 141′ to be set in different positions in relation to the connecting portion 142 and in extension also in relation to the second portion 141″. Likewise, such rotational symmetry may allow the second portion 142″ to be set in different positions in relation to the connecting portion 142 and in extension also in relation to the first portion 141′.

With reference to FIGS. 51a-51c, a procedure of insertion of the remote unit 140 in a tissue portion 610 will be described. The remote unit 140 may be oriented such that a length direction 631 of the second portion 141″ points downwards into the hole 611. Preferably, the second portion 141″ is positioned such that it is inserted close to an edge of the hole 611. The second portion 141″ may then be inserted partially through the hole 611, until the point where the first portion 141′ abuts the first tissue surface 616. Here, a 900 rotational displacement between the first portion 141′ and the second portion 141″, as described above, will allow a relatively large portion of the second portion 141″ to be inserted before the first portion 141′ abuts the first tissue surface 616. Subsequently, the remote unit 140 may be pivoted to slide or insert the remaining portion of the second portion 141″ through the hole 611. While inserting the remaining portion of the second portion 141″, the tissue may naturally flex and move to give way for the second portion 141″. Upon having fully inserted the second portion 141″ through the hole 611, such that the second portion 141″ is completely located on the other side of the tissue portion 610, the tissue may naturally flex back.

With reference to FIGS. 56a-561, embodiments of an implantable energized medical device 140, which may be referred to as a remote unit in other parts of the present disclosure, will be described. As illustrated, these implantable energized medical devices have a second portion being shaped in a particular manner in order to facilitate removal of the implantable energized medical device once it has been implanted for a period of time and fibrotic tissue has begun to form around the second portion. It is hereby disclosed that these types of second portions, as illustrated in FIGS. 56a-561 c, and as disclosed below, may be combined with any of the other features of the implantable energized medical device discussed in the present disclosure.

The device 140 is configured to be held in position by a tissue portion 610 of a patient. The device 140 comprises a first portion 141′ configured to be placed on a first side 612 of the tissue portion 610, the first portion 141′ having a first cross-sectional area in a first plane and comprising a first surface configured to face and/or engage a first tissue surface 616 of the first side 612 of the tissue portion 610.

The device 140 further comprises a second portion 141″ configured to be placed on a second side 618 of the tissue portion 610, the second side 618 opposing the first side 612, the second portion 141″ having a second cross-sectional area in a second plane and comprising a second surface configured to engage a second tissue surface 622 of the second side 618 of the tissue portion 610. The device 140 further comprises a connecting portion 142 configured to be placed through a hole in the tissue portion 610 extending between the first and second sides 612, 618 of the tissue portion 610. The connecting portion 142 here has a third cross-sectional area in a third plane. The connecting portion 142 is configured to connect the first portion 141′ to the second portion 141″. In the illustrated embodiment, a connecting interface 630 between the connecting portion 142 and the second portion 141″ is arranged at an end of the second portion 141″.

The first portion 141′ may have an elongated shape. Similarly, the second portion 141″ may have an elongated shape. However, the first portion 141′ and/or second portion 141″ may assume other shapes, such as a flat disk e.g. having a width and length being larger than the height, a sphere, an ellipsoid, or any other polyhedral or irregular shape, some of these being exemplified in FIGS. 42-44.

To provide a frame of reference for the following disclosure, and as illustrated in FIGS. 51g,51h and 51i, a first direction 631 is here parallel to the line A-A, to the second plane, and to a length of the second portion 141″. A second direction 633 is here parallel to the line B-B, to the second plane, and to a width of the second portion 141″. The second portion 141″ has a first end 632 and a second end 634 opposing the first end 632. The length of the second portion 141″ is defined as the length between the first end 632 and the second end 634. The length of the second portion 141″ is furthermore extending in a direction being different to the central extension C1 of the connecting portion 142. The first end 632 and second end 634 are separated in a direction parallel to the second plane. Similarly, the first portion 141′ has a length between a first and a second end, the length extending in a direction being different to the central extension C1 of the connecting portion 142.

The first portion 141′, connecting portion 142 and second portion 141″ may structurally form one integral unit. It is however also possible that the first portion 141′ and the connecting portion 142 structurally form one integral unit, while the second portion 141″ form a separate unit, or, that the second portion 141″ and the connecting portion 142 structurally form one integral unit, while the first portion 141′ form a separate unit.

Additionally, or alternatively, the second portion 141″ may comprise a removable and/or interchangeable portion 639 as described in other parts of the present disclosure.

In the following paragraphs, some features and properties of the second portion 141″ will be described. It is however to be understood that these features and properties may also apply to the first portion 141′.

The second portion 141″ has an intermediate region 638, and a distal region 640. A proximal region may be present, as described in other parts of the present disclosure, The intermediate region 638 is defined by the connecting interface 630 between the connecting portion 142 and the second portion 141″, and the distal region 640 extends from the connecting interface 630 between the connecting portion 142 and the second portion 141″ to the second end 634.

The first surface 614 configured to face and/or engage the first tissue surface 616 of the first side 612 of the tissue portion 610 may be substantially flat. In other words, the first portion 141′ may comprise a substantially flat side facing towards the tissue portion 610. Furthermore, an opposing surface of the first portion 141′, facing away from the tissue portion 610, may be substantially flat. Similarly, the second surface 620 configured to engage the second tissue surface 622 of the second side 618 of the tissue portion 610 may be substantially flat. In other words, the second portion 141″ may comprise a substantially flat side facing towards the tissue portion 610. Furthermore, an opposing surface of the second portion 141″, facing away from the tissue portion 610, may be substantially flat.

The second portion 141″ may be tapered from the first end 632 to the second end 634, thus giving the second portion 141″ different heights and/or widths along the length of the second portion 141″. The second portion may also be tapered from each of the first end 632 and second end 634 towards the intermediate region 638 of the second portion 141″.

Still referring to FIGS. 51a-511, the second portion 141″ and connecting portion 142 here form a connecting interface 630. Furthermore, the second portion 141″ has a lengthwise cross-sectional area along the first direction, wherein a second lengthwise cross-sectional area 690 is smaller than a first lengthwise cross-sectional area 689 and wherein the first lengthwise cross-sectional area 689 is located closer to the connecting interface 630 with regard to the first direction 631. Hereby, a tapered second portion is formed, being tapered towards the second end 634. The lengthwise cross-sectional area of the second portion 141″ may decrease continuously from an end of the intermediate region 638 towards the second end 634, as illustrated for example in FIG. 51g. The decrease may be linear, as illustrated for example in FIG. 51g. However, other types of decreasing lengthwise cross-sectional areas are possible, such as a parabolic, exponential, stepwise, or stepwise with radiused edges between each step thus forming a smooth rounded contour.

FIGS. 51e and 51f illustrate how the lengthwise cross-sectional area decrease over the length of the second portion 141″ towards the second 634, as viewed along the line A-A. FIG. 51e illustrate the first lengthwise cross-sectional area 689, and FIG. 51f illustrate the second lengthwise cross-sectional area 690.

In some embodiments, the lengthwise cross-sectional area may decrease over a majority of the length of the second portion towards the second end 634. In some embodiments, a decrease of the lengthwise cross-sectional area over at least of the length of the second portion towards the second end 634 may be sufficient. In the example illustrated in FIG. 51g, the lengthwise cross-sectional area decrease over about 85% of the length of the second portion.

With the second portion 141″ having rotational symmetry along the first direction 631, as illustrated for example in FIG. 51d, the shape of the second portion 141″ may be conical.

As illustrated in FIG. 51h, the second portion 141″ may have an upper surface, which include the second surface 620 configured to engage a second tissue surface of the second side of the tissue portion as discussed in other parts of the present disclosure, wherein the upper surface or second surface 620 is substantially flat and parallel to the second plane. In some embodiments the upper surface may be substantially perpendicular to the central extension C1 of the connecting portion 142. Hereby, the second surface may be configured to lay flat against the second side of the tissue portion. In such embodiments, a lower surface of the second portion 141″, opposite the second surface 620 and facing away from the first portion 141′, may be configured to taper towards the second end 634, thus achieving the decreasing lengthwise cross-sectional area along the first direction 631 towards the second end 634.

FIG. 51i illustrate an embodiment wherein the lengthwise cross-sectional area decrease in a stepwise manner towards the second end 634 of the second portion 141″. Here, the second portion 141″ has three major segments 692, 693, 694 having substantially constant diameter and each respective diameter being smaller moving towards the second end 634, being connected by intermediate segments 695, 696, wherein the diameter decreases along the first direction 631. Other variations of major segments having substantially constant diameter, and intermediate segments, having a decreasing diameter along the first direction 632, are possible, such as at least two major segments connected by a single intermediate segment with decreasing diameter, at least four major segments connected by three intermediate segments with decreasing diameter, and so on.

Referring now to FIGS. 51j-511, an implantable energized medical device similar to the one illustrated in FIG. 51h is illustrated. As can be seen in the perspective view of FIG. 51j, the second portion 141″ has a decreasing lengthwise cross-sectional area towards the second end. The upper surface 697 is also visible in this view, being substantially flat and providing a contact area to the second tissue surface 622. The first lengthwise cross-sectional area 689 is larger than the second cross-sectional area 690, as can be seen in FIGS. 51k-511, and the first lengthwise cross-sectional area 689 is located closer to the connecting interface between the connecting portion 142 and the second portion 141″ with regard to the first direction.

Referring now to FIG. 51m, an implantable energized medical device which may incorporate one or several of the features described in conjunction with FIGS. 33-511, and which may be referred to as a remote unit in other parts of the present disclosure, will be described. The remote unit 140 is configured to be held in position by a tissue portion 610 of a patient. The remote unit 140 comprises a first portion 141′ configured to be placed on a first side 612 of the tissue portion 610, the first portion 141′ having a first cross-sectional area in a first plane and comprising a first surface configured to face and/or engage a first tissue surface of the first side 612 of the tissue portion 610. The remote unit 140 further comprises a second portion 141″ configured to be placed on a second side 618 of the tissue portion 610, the second side 618 opposing the first side 612, the second portion 141″ having a second cross-sectional area in a second plane and comprising a second surface configured to engage a second tissue surface of the second side 618 of the tissue portion 610. The remote unit 140 further comprises a connecting portion 142 configured to be placed through a hole in the tissue portion 610 extending between the first and second sides 612, 618 of the tissue portion 610. The connecting portion 142 is configured to connect the first portion 141′ to the second portion 141″.

The connecting portion 142 and the second portion 141″ are configured to form a unit having a central axis C2 extending from a first end 650 of said unit to a second end 651 of said unit, the first end 650 being proximal to the first portion 141′ and the second end 651 being distal to the first portion 141′. The first end 650 may generally be defined as the interface between the connecting portion 142 and the first portion 141′.

A physical footprint of the unit perpendicular to the central axis C2 decreases continuously or stepwise from the first end 650 to the second end 651 of said unit. Here, the physical footprint 652 is smaller than the physical footprint 653 which is more proximal to the first end 650, the physical footprint 653 in turn being smaller than the physical footprint 654 which is even more proximal to the first end 650. The illustrated footprints 652, 653, 654 may be cross-sectional areas which are determined in a plane perpendicular to the central axis C2. The footprints 652, 653, 654 may also be seen as the extension of the unit in a plane perpendicular to the central axis C2.

In embodiments where the unit comprises one or more bends or one or more angled sections, the physical footprint shall preferably decrease continuously or stepwise from the first end 650 to the second end 651 of the unit also along such bends or angled sections.

By decreasing the physical footprint along the central axis C2, removal of the remote unit 140 may be facilitated. In particular, the device 10 may more easily slide out of scar tissue which has formed around the implanted remote unit 140.

The connecting portion 142 and the second portion 141″ may be configured to reversibly connect to each other to form the unit. Such a connection may be a snap-fit connection, a magnetic type connection, a threaded connection, or a combination thereof, as described in other parts of the present disclosure. An irreversible connection between the connecting portion 142 and the second portion 141″ is also possible. In this sense, the term “irreversible” shall be understood as a connection which cannot be disengaged without damage or irreversible damage. It is also possible that the connecting portion 142 and the second portion 141″ are formed as a single body forming the unit. In such cases there are no seal or interface between the connecting portion 142 and the second portion 141″.

The unit here comprises an angled section forming a bend in the unit. The bend being about 900 as measured from the first end 650 to the second end 651. Hereby, a secure position is achieved, and a smaller vertical footprint, i.e. the space occupied by the device in a direction inwards to the center of the patient, may be achieved. The bend may be between 150 and 165°, such as between 300 and 150°, such as between 450 and 135°, such as substantially 90°.

Referring now to FIGS. 51n-51p, an implantable energized medical device which may incorporate one or several of the features described in conjunction with FIGS. 33-51m, and which may be referred to as a remote unit in other parts of the present disclosure, will be described.

The remote unit 140 is configured to be held in position by a tissue portion 610 of a patient. The remote unit 140 comprises a first portion 141′ configured to be placed on a first side 612 of the tissue portion 610, the first portion 141′ having a first cross-sectional area in a first plane and comprising a first surface configured to face and/or engage a first tissue surface of the first side 612 of the tissue portion 610. The remote unit 140 further comprises a second portion 141″ configured to be placed on a second side 618 of the tissue portion 610, the second side 618 opposing the first side 612, the second portion 141″ having a second cross-sectional area in a second plane and comprising a second surface configured to engage a second tissue surface of the second side 618 of the tissue portion 610. The remote unit 140 further comprises a connecting portion 142 configured to be placed through a hole in the tissue portion 610 extending between the first and second sides 612, 618 of the tissue portion 610. The connecting portion 142 is configured to connect the first portion 141′ to the second portion 141″.

The remote unit 140 further comprises a hermetic seal arrangement, which may cover the first portion 141′, the connecting portion 142 and the second portion 141″, as visualized by hermetic seal arrangement 656a. Such hermetic seal arrangement may for example be achieved by a housing made of a metal, such as titanium. The hermetic seal arrangement may also cover only the connecting portion 142 and the second portion 141″, as visualized by hermetic seal arrangement 656b. Such hermetic seal arrangement may be achieved by the flexible structure 655 being sealed with regard to the first portion 141′, and further wherein the flexible structure 655 is either joined with the second portion 141″ in a sealing manner, or formed as an integral unit with the second portion 141″.

Any entry to the connecting portion 142 and/or the second portion 141″, may be achieved by means of a sealed entry (not shown). Such sealed entry can for example be achieved by a portion of an outside of connecting portion 142 and/or the second portion 141″ comprising a ceramic portion integrated in, or brazed to, the material of the connecting portion 142 and/or the second portion 141″ respectively. In such cases, the material of the connecting portion 142 and/or the second portion 141″ is preferably a metal, such as titanium. At least one metallic lead or conduit may travel through the sealed entry for transferring energy, information or fluid respectively from an outside of the remote unit 140, also known as the wet side, to an inside of the remote unit 140. The at least one metallic lead may in turn be integrated in, or brazed to, the ceramic portion. Thus, the at least one metallic lead can pass the sealed entry without being further insulated, such that the sealed entry can enable the transfer of electrical energy, information, or fluid, through a wall of titanium and ceramics, such that the connecting portion 142 and/or the second portion 141″ can be hermetically enclosed by the hermetical seal arrangement which reduces the risk of any fluid diffusing into the remote unit 140, and in particular into the connecting portion 142 and/or the second portion 141″.

Here, the connecting portion 142 comprises a flexible structure 655 enabling the connecting portion 142 to flex. As can be seen in FIG. 51, the connecting portion 142 may flex to accommodate for the thickness of the tissue portion 610, in this particular case the second portion 141″ is flexing downwards and away from the first portion 141′. However, the flexible structure 655 may be configured to allow flexing in any and all directions. In some embodiments, the flexible structure 655 may be configured to allow roll, pitch, and/or upwards and downwards movement with regard to the first portion 141′ and/or second portion 141″. As can be seen in FIG. 51p, the connecting portion 142 may flex to accommodate for the thickness of the tissue portion 610, in this particular case the first portion 141′ is flexing downwards and towards the second portion 141″.

The flexible structure 655 here comprises a bellows, which may be annularly fixated by means of soldering or welding to the first portion 141′ and/or the second portion 141″. The bellows may be a metallic bellows, and more specifically may be a titanium bellows. The flexible structure 655 may thus be flexible by means of elasticity of the metal or the titanium. Metals are generally dense which is advantageous as fluids do not easily diffuse through the metal. This reduces the risk that gas or fluid diffuses into the remote unit 140.

The bellows of the flexible structure 655 may assume a relaxed state, i.e. where the structure is not biased. In such relaxed state the flexible structure 655 may have a length L1 as measured from the first portion 141′ to the second portion 141″. Once the flexible structure 655 is compressed, the length of the flexible structure 655 may decrease to a length L2. Conversely, if the flexible structure 655 is pulled, the length of the flexible structure 655 may increase to L3, being larger than both L1. Depending on the corrugated structure of the bellows, e.g. the dimensions of the corrugations and their frequency along the length of the bellows, different degrees of flexibility may be achieved.

The bellows comprise lowered portions and elevated portions. The lowered portions and elevated portions enable at least one of compression, expansion and flexing of the bellows. By compressing or expanding one side of the bellows, flexing of the first portion 141′ or second portion 141″ may be achieved.

If the bellows is made from a metal, the metal may be welded to form the corrugations of the bellows. Furthermore, the bellows, or the flexible structure 655, may form part of the hermetic seal arrangement.

The flexible structure 655 may have a substantially cylindrical shape, as illustrated in FIGS. 51n-51p. Such shape may provide for that flexing is available in all directions with little to no variation in resistance depending on the flexing direction.

Referring now to FIGS. 51q-51t, an implantable energized medical device similar to the one described in conjunction with FIGS. 51n-51p, and which may incorporate one or several of the features described in conjunction with FIGS. 33-51n, and which may be referred to as a remote unit in other parts of the present disclosure, will be described. In particular, it is shown how the remote unit 140 may be inserted into a hole 611 in a tissue portion 610 of a patient.

Owing to the flexible structure 652 of the connecting portion 142, the first portion 141′ and second portion 141″ can be separated to increase the distance between respective ends 657,659 of the first portion 141′ and second portion 141″. Hereby, the second portion 141″ can be inserted into the hole 611 in the tissue portion 610 without being hindered by the first portion 141′ abutting the tissue portion 610, as shown in FIG. 51r. Once a sufficiently large portion of the second portion 141″ has been inserted through the hole 611, the remote unit 140 can be rotated to achieve the desired position in the tissue portion 610.

Referring now to FIGS. 51u-51z, an implantable energized medical device which may incorporate one or several of the features described in conjunction with FIGS. 33-51r, and which may be referred to as a remote unit in other parts of the present disclosure, will be described. It should be noted that FIGS. 51u-51z are schematic.

In each of the embodiments illustrated in FIGS. 51u-51z, the remote unit 140 comprises a first portion 141′, a connecting portion 142, and a second portion 141″. The remote unit 140 is placed in a hole in a tissue portion 610. The remote unit 140 comprises an electric motor 661 wherein at least part of the electric motor is arranged within the connecting portion 142. The connecting portion 142 may be defined by an imaginary boundary 663 defined by the first surface 614 of the first portion 141′ extending through the connecting portion 142. Here, the imaginary boundary 663 lines up with the first side 612 of the tissue portion 610. The connecting portion 142 may be further defined by an imaginary boundary 667 defined by the second surface 620 of the second portion 141″ extending through the connecting portion 142. Here, the imaginary boundary 667 lines up with the second side 618 of the tissue portion 610.

Furthermore, in each of the illustrated embodiments of FIGS. 51u-51z, the electric motor 661 is preferably oriented such that its longest dimension aligns with a longest dimension of the remote unit 140, in this case being a dimension extending from the first portion 141′ to the second portion 141″ as indicated by the reference sign LD.

Referring first to FIG. 51u, the electric motor 661 is fully arranged within the connecting portion 142.

In FIG. 51v, the electric motor 661 is arranged such that it extends through the connecting portion into the first portion 141′, past the imaginary boundary 663, and such that it extends through the connecting portion into the second portion 141″, past the imaginary boundary 667.

In FIG. 51w, the electric motor 661 is arranged such that it is contained within the imaginary boundary 663, in the connecting portion 142, and such that it extends through the connecting portion into the second portion 141″, past the imaginary boundary 667.

In FIG. 51x, a gear arrangement 669 is operatively connected to the electric motor 661.

The gear arrangement may be located in the second portion 141″ or the connecting portion 142. The electric motor 661 may extend into the second portion 141″, past the imaginary boundary 667, as illustrated, or it may be located within the connecting portion 142 within the imaginary boundary 667.

The gear arrangement 669 is configured to transfer mechanical force from the electric motor to an implantable body engaging portion (not shown) being external to the remote unit 140, or to an actuating element in e.g. a pump (not shown). The gear arrangement 669 may be a worm drive, as illustrated. The gear arrangement 669 may further be configured to transfer movement from the electrical motor 661 to a different plane or axis than what is provided by the electric motor 661 itself. In the illustrated embodiment, the gear arrangement 669 transfers movement from the electrical motor 661 to a new axis being substantially perpendicular to the axis provided by the electric motor 661.

In FIG. 51y, an embodiment similar to the one described in conjunction with FIG. 51x is shown. Here, a gear arrangement 669 is operatively connected to an electric motor 661. The electric motor 661 is arranged such that it is contained within the connecting portion 142, within the imaginary boundaries 663 and 667. However, the gear arrangement 669 is arranged in the second portion 141″.

In FIG. 51z, a gear arrangement 669 is operatively connected to the electric motor 661.

The gear arrangement 669 as well as the electric motor 661 is arranged such that they are contained within the connecting portion 142, within the imaginary boundaries 663, 667.

Without reference to any particular figure, it is herein further disclosed that the implantable energized medical remote unit 140 may comprise the first portion configured to be placed on a first side of the tissue portion, the first portion comprising a first surface configured to face a first tissue surface of the first side of the tissue portion, the first portion being further configured to connect, directly or indirectly, to a second portion, as disclosed in other parts of the present disclosure, placed on a second side of the tissue portion opposing the first side, wherein the first portion comprises an internal wireless energy transmitter configured to transmit energy wirelessly to the second portion. Such first portion may be configured to connect to the second portion via a connecting portion as disclosed in other parts of the present disclosure. The connecting portion may form part of such remote unit 140, and may be integrally formed with the first portion, or may be a separate component with regard to the first portion, wherein the connecting portion is configured to connect to the first portion. In other words, one embodiment of the remote unit 140 may consist of the first portion only, omitting the second portion and optionally omitting the connecting portion.

FIG. 51aa shows a cross-sectional view of an implantable energized medical device, which may incorporate one or several of the features described in conjunction with FIGS. 33-51z, and which may be referred to as a remote unit in other parts of the present disclosure.

The second portion 141″ of the remote unit 140 comprises or forms a reservoir 671 for holding a fluid, and remote unit 140 further comprises a sealed container C configured to protrude into the reservoir 671. An actuator A is connected to the sealed container C, the actuator A being configured to expand or retract the sealed container C to change the volume of the sealed container C for pumping fluid to or from the reservoir 671.

The reservoir 671 is configured to hold the fluid to be pumped. The fluid is preferably a biocompatible incompressible liquid, such as a saline solution, but could in the alternative be an oil-based liquid, such as a silicone oil, or a gas. The sealed container C is configured to protrude into the reservoir 671, such that a wall of the sealed container forms a portion of a wall enclosing the reservoir 671. The sealed container C comprises a first movable wall portion 672 forming a portion of the wall of the reservoir 671. The movable wall portion 672 being a portion of the sealed container C protruding into the reservoir 671. The actuator A is directly or indirectly connected to the first movable wall portion 672, for moving the movable wall portion 672, for altering a volume of the sealed container C and a volume of the reservoir 671, for pumping the fluid to or from the reservoir 671.

The sealed container C may comprise a first portion (as shown) and a second portion (not shown), where movement of the first movable wall portion 672 causes movement of a second movable wall portion, altering a volume of the second portion of the sealed container C, such that the volume change of the sealed container C is less than the volume change of the reservoir 671, when the volume of the reservoir 671 is altered for pumping fluid to or from the reservoir 671. When the volume of the reservoir 671 is reduced and expanded, the reservoir 671 functions as a pump for moving fluid to and from a body engaging portion, i.e. an additional implant in the patient's body

The first and second portions of the sealed container may be mirrored and identical, and as such, in a relaxed state, have the same volume. The pressure in the sealed container remains substantially the same all the time as the volume of the sealed container C remains substantially the same when the volume of the reservoir 671 is altered for pumping fluid to or from the reservoir 671.

In the embodiment shown in FIG. 51aa, the sealed container C is annular, and the center of the sealed container C is annularly fixated to outer portions of the reservoir 671. As the actuator moves the first movable wall portion 672 causing the sealed container C to expand, a second movable wall portion, if present, moves the same distance, contracting the second portion of the sealed container C, such that the volume in the sealed container remains the same when the volume of the reservoir 671 is altered for pumping fluid to or from the reservoir 671. The sealed container C is substantially rigid in directions other than the length extension LE of the sealed container C, such that the diameter of the sealed container is substantially constant and the volume of the sealed container only changes as a result of the first and second movable wall portions moving in the direction of the length extension of the sealed container C.

In the embodiment shown in FIG. 51aa, the sealed container C comprises a titanium bellows, and the titanium bellows is annularly fixated by means of soldering or welding to the to reservoir or second portion 141″.

In the embodiment shown in FIG. 51aa, the volume of the sealed container C is altered less than 10% when the volume of the reservoir is altered for pumping fluid to or from the reservoir 671. In particular, the volume of the sealed container C may be altered less than 5% when the volume of the reservoir 671 is altered for pumping fluid to or from the reservoir 671.

In the embodiment shown in FIG. 51aa, the walls of the sealed container C comprises lowered portions and elevated portions. The lowered portions and elevated portions enable at least one of compression and expansion of the sealed container C. In particular, in the embodiment shown in FIG. 51aa the sealed container C comprises a bellows enabling the contraction and expansion of the sealed container C, by means of the elasticity of the bellows making the bellows flexible. In particular, in the embodiment shown in FIG. 51aa, the bellows is a metal bellows, in particular a titanium bellows. The sealed container C in the form of a titanium bellows is thus flexible by means of the elasticity of the titanium.

As the sealed container C is a titanium bellows 452, at least a portion of the first movable wall portion 672 being in contact with the fluid in the reservoir 671 comprises metal, namely the titanium. Metals are generally dense which is advantageous as fluids do not diffuse through the metal as easy. This reduces the risk that gas diffuses from the sealed container C or that fluids diffuse into the sealed container C. In the embodiment shown in FIG. 51aa, the entire wall enclosing the sealed container C is made from metal, in particular titanium. In alternative embodiments it is however conceivable that a portion of the wall of the sealed container C is made from a flexible or elastic polymer material, such as a silicone-based material or a polyurethane-based material. In embodiments in which the sealed container has a wall made from composite of metallic or non-metallic materials, the non-metallic materials could be provided as a layer or a coating applied or sprayed onto the metal. In some embodiments, at least 50% of the area of the wall enclosing the sealed container C comprises metal and in alternative embodiments at least 80% of the area of the wall enclosing the sealed container C comprises metal, and in yet alternative embodiments, at least 90% of the area of the wall enclosing the sealed container C comprises metal.

The sealed container C may be configured to enclose a gas, such as helium or air. More specifically, the sealed container C may be configured to enclose a gas having a pressure exceeding standard atmospheric pressure (atm), i.e. 101,325 Pa. The sealed container C in the embodiment of FIG. 51aa is hermetically enclosed by a metallic layer such that the gas enclosed in the sealed container C is hermetically enclosed by a metallic layer.

The remote unit 140 may further comprise a fluid conduit 673 for connecting the reservoir 671 to a body engaging portion 676 of an implant configured for receiving the fluid pumped from the reservoir 671. The body engaging portion 676 is here an implantable constriction device in a state in which the implantable constriction device is constricting a luminary organ U and thereby restricts the flow of fluid through the luminary organ U. The implantable constriction device comprises a surrounding structure 677 having a periphery surrounding the luminary organ U when implanted. The surrounding structure 677 comprises two support elements 678a, 678b connected to each other for forming the surrounding structure 677. The first support element 678a is configured to support a first operable hydraulic constriction element 679. The first operable hydraulic constriction element 679 is configured to constrict the luminary organ U for restricting the flow of fluid therethrough and configured to release the constriction of the luminary organ U upon request. The first and second support elements 678a, 678b each comprises a curvature adapted for the curvature of the luminary organ U such that the implantable constriction device fits snuggly around the luminary organ U such that the distance that the operable hydraulic constriction elements 679 needs to expand to constrict the luminary organ U is kept at a minimum.

All foreign matter implanted into the human body inevitably causes an inflammatory response. In short, the process starts with the implanted medical device immediately and spontaneously acquiring a layer of host proteins. The blood protein-modified surface enables cells to attach to the surface enabling monocytes and macrophages to interact on the surface of the medical implant. The macrophages secrete proteins that modulate fibrosis and in turn developing the fibrosis capsule around the foreign body. In practice, a fibrosis capsule is a dense layer of excess fibrous connective tissue. On a medical device implanted in the abdomen, the fibrotic capsule typically grows to a thickness of about 0.5 mm-2 mm, and is substantially inelastic and dense.

Fluid is conducted from the reservoir 671 to the operable hydraulic constriction element 679 such that the implantable constriction device constricts the luminary organ U and thereby restricts a flow of fluid through the luminary organ U. As a first portion of the sealed container C is expanded and the second portion of the sealed container, if present, is compressed, the volume of the sealed container C remains the same throughout the shift, and as such, the actuator A does not need to work against a changing pressure in the sealed container C. The volume of the reservoir 671 when the sealed container C is in its most expanded state may be less than 50% of the volume of the reservoir 671 when the sealed container C is in its most compressed state.

The actuator A may comprise an electrical motor M configured to convert electrical energy to a rotating mechanical force. The motor M may be connected to a transmission configured to receive mechanical force and reduce the speed and increase the force of the received mechanical force. In the embodiment of FIG. 51aa, the transmission is in the form of a gear arrangement G configured to increase the torque of the mechanical force created by the electrical motor M and to deliver a force with a higher torque. Consequently, low torque may be provided by the motor M, i.e. a relatively small force with high angular velocity, which is transferred to the gear arrangement G to achieve a relatively large force with low angular velocity. The gear arrangement is in turn connected to a further transmission configured to transform the received rotating mechanical force into a liner mechanical force. The further transmission comprises a shaft 674 connected to the force output of the gear arrangement G. The shaft comprises outer threads 674t adapted to engage inner threads of a nut portion 675 in the form of a hollow shaft having inner threads, such that the interaction between the threaded shaft 674, 674t and the threaded inner portion of the nut portion 675 transforms the radially rotating force generated by the motor M and the gear system G, to a linear force acting in the axial direction of the shaft 674. The nut portion 675 is fixated to, or integrated with, the first movable wall portion 672 of the sealed container C.

The sealed container C may further comprise a first and a second connecting member for connecting the first movable wall portion 672 to the second movable wall portion (not shown), such that the second movable wall portion moves in synchronization with the first movable wall portion 672, as the operation device operates the first movable wall portion 672. The first and second connecting members could be metal rods, such as titanium rods welded or soldered to the inner surfaces of the first and second movable wall portions respectively.

The actuator A is further arranged within the sealed container C, thus being protected from fluids in the reservoir 671 by means of the hermetic property of the sealed container C.

The reservoir 671 may have an oval cross-section, more specifically an elliptic cross-section, and even more specifically a circular cross-section. Having a circular cross-section enables the reservoir 671 to have the same cross-sectional shape as the sealed container C, which may also have an oval cross-section, more specifically an elliptic cross-section, and even more specifically a circular cross-section, which means that the distance between the wall of the bellows of the sealed container C can be made really small for reducing the space occupied by the remote unit 140 in the body of the patient.

The sealed container C may further comprise a sealed entry in the form of a portion of the wall of the sealed container C comprising a ceramic portion integrated in, or brazed to, the metal or titanium of the sealed container C. At least one metallic lead may travel through the sealed entry for transferring electrical energy or information from within the sealed container C to the environment outside the sealed container C, also known as the wet side. The at least one metallic lead may in turn be integrated in, or brazed to, the ceramic portion. Thus, the at least one metallic lead can pass the sealed entry without being further insulated, such that the sealed entry can enable the transfer of electrical energy or information through a wall of titanium and ceramics, such that the sealed container C can be hermetically enclosed by titanium and ceramics which reduces the risk of any fluid diffusing into the sealed container C.

As shown in FIG. 51aa, the remote unit 140 may further comprise an injection port 681 configured to receive a needle or other means for transferring fluid to or from the remote unit 140, in particular to or from the reservoir 671. The injection port 681 is connected to an internal conduit 682 which is fluidly connected to the reservoir 671 via a port 683. The injection port 681 may comprise a membrane configured to form a seal around the needle or other means for transferring fluid when penetrated.

Referring now to FIGS. 51ab-51aq, embodiments of a system comprising an implantable energized medical device, which may incorporate one or several of the features described in conjunction with FIGS. 33-51aa, and which may be referred to as a remote unit in other parts of the present disclosure.

Each system depicted in FIGS. 51ab-51ap comprises an implantable energized medical remote unit 140 configured to be held in position by a tissue portion 610 of a patient, the remote unit 140 comprising: a first portion 141′, a second portion 141″, and a connecting portion 142, as described elsewhere in the present disclosure. The system may further comprise at least one of the following components: an implantable energy storage unit 304, an implantable reservoir 107 configured to hold a fluid, a body engaging implant 676 configured to at least one of stretch, contract, expand, stimulate and exert a force on body tissue or a body organ, an implantable pump 104 configured to transfer fluid to and from the implantable reservoir and the body engaging implant respectively via a conduit, and an implantable electric motor 661 connected to the implantable energy storage unit 304, the implantable electric motor 661 being configured to operate the implantable pump 104. The embodiments differ mainly in the arrangement and location of the components, in particular whether the components are arranged within the device, i.e. in the first portion 141′, connecting portion 142 and/or second portion 141″, or whether they are arranged external to the device, i.e. outside of the first portion 141′, connecting portion 142 and/or second portion 141″.

As illustrated, the implantable pump 104 may be connected to the implantable reservoir 107 via a fluid conduit.

The expression “arranged externally” shall in this context be understood as a component being configured to be located outside of the remote unit 140. Furthermore, such component may be configured to be implanted in a location in the patient's body which is remote to the location of the implanted remote unit 140.

Furthermore, each system comprises an internal component 685 arranged in the remote unit 140, wherein the internal component 685 may have capabilities of at least one of receiving wireless energy, transmitting wireless energy, receiving communication signals, and transmitting communication signals. The internal component 685, although here illustrated as a single unit, may comprise several units.

The illustrated embodiments are purely schematic, and lines connecting components may symbolize one or several metallic leads for transferring energy and/or for transferring communication signals (such may be the case for lines between the implantable energy storage unit 304 and the implantable electric motor 661). They may also symbolize a shaft or magnetic coupling configured to transfer force or movement (such may be the case for lines between the implantable electric motor 661 and the implantable pump 104). It shall also be noted that the lines connecting components does not necessarily imply that the components are physical separated and connected by e.g. metallic leads or shafts. They may instead simply be seen as an indication that the components are connected electrically or mechanically. For example, in the case of the implantable electric motor 661 and the implantable pump 104, these two components, although illustrated as being separate and connected by a line, may form part of a single unit which integrates the implantable electric motor 661 with the implantable pump 104 to achieve the functionality of a pump. Furthermore, the implantable pump 104 may form part of the implantable reservoir 104, and in particular the implantable pump 104 may be arranged at least partly inside or in connection with the implantable reservoir 104.

Furthermore, the location of the components in relation to the remote unit 140, internally and externally, as well as the length of the illustrated lines and conduits, shall not be seen as limited by the illustrated embodiments. In contrast, for example, the length of lines and conduits may be shorter or longer than depicted. Similarly, illustrated components may be located closer to or further away from each other, and/or closer to or further away from the remote unit 140, and/or in other locations or portions of the remote unit 140. Furthermore, the entrance and exit of lines or conduits with regard to the remote unit 140 are schematic and only exemplary.

Furthermore, although not shown in the illustrated embodiments, a conduit in fluid connection with the reservoir may be arranged internally and/or externally to the remote unit 140 for introducing and/or removing fluid from the reservoir. The conduit may be connected to an injection port in the remote unit 140.

A gear arrangement may be included in the system, preferably arranged in proximity of the electric motor 661. The gear arrangement, if present, is operatively connected to the electric motor and configured to reduce the velocity and increase the force of movement generated by the electric motor 661. Thus, if the electric motor 661 is arranged externally with the regard to the remote unit 140, the gear arrangement may be arranged externally as well, or internally, i.e. within the remote unit 140, and vice versa.

An advantage of having one or more components externally arranged to the remote unit 140 is that load distribution may be improved. This is particularly important since one of the objects of the remote unit 140 is to achieve and maintain a secure placement in the patient's body. By distributing weight to other parts of the patient's body, the risk of detachment of the remote unit 140 from its implanted position may be decreased. Furthermore, it may be advantageous to distribute heat generation from one or more of the components to particular parts or regions or several parts or regions of the patient's body.

Referring first to FIG. 51ab, the system comprises, externally arranged with regard to the remote unit 140, an implantable energy storage unit 304, an implantable electric motor 661, an implantable reservoir 107, and an implantable pump 104.

Referring now to FIG. 51ac, the system comprises, externally arranged with regard to the remote unit 140, an implantable electric motor 661, an implantable reservoir 107, and an implantable pump 104. Furthermore, the system comprises an implantable energy storage unit 304 arranged in the remote unit 140.

Referring now to FIG. 51ad, the system comprises, externally arranged with regard to the remote unit 140, an implantable energy storage unit 304, an implantable reservoir 107, and an implantable pump 104. Furthermore, the system comprises an implantable electric motor 661 arranged in the remote unit 140.

Referring now to FIG. 51ae, the system comprises, externally arranged with regard to the remote unit 140, an implantable energy storage unit 304, an implantable electric motor 661, and an implantable pump 104. Furthermore, the system comprises an implantable reservoir 107 arranged in the remote unit 140.

Referring now to FIG. 51af, the system comprises, externally arranged with regard to the remote unit 140, an implantable energy storage unit 304, an implantable electric motor 661, and an implantable reservoir 107. Furthermore, the system comprises an implantable pump 104 arranged in the device 104.

Referring now to FIG. 51ag, the system comprises, externally arranged with regard to the remote unit 140, an implantable energy storage unit 304 and an implantable electric motor 661. It should be noted that this embodiment does not necessarily comprise an implantable reservoir 107 nor an implantable pump 104. The implantable electric motor 661 is here operatively connected to the body engaging implant 676.

Referring now to FIG. 51ah, the system comprises, externally arranged with regard to the remote unit 140, an implantable reservoir 107 and an implantable pump 104. Furthermore, the system comprises an implantable energy storage unit 304 and an implantable electric motor 661, arranged in the device 104.

Referring now to FIG. 51ai, the system comprises, externally arranged with regard to the remote unit 140, an implantable energy storage unit 304 and an implantable pump 104. Furthermore, the system comprises an implantable electric motor 661 and an implantable reservoir 107, arranged in the remote unit 140.

Referring now to FIG. 51aj, the system comprises, externally arranged with regard to the remote unit 140, an implantable energy storage unit 304 and an implantable electric motor 661. Furthermore, the system comprises an implantable reservoir 107 and an implantable pump 104, arranged in the remote unit 140.

Referring now to FIG. 51ak, the system comprises, externally arranged with regard to the remote unit 140, an implantable electric motor 661 and an implantable reservoir 107. Furthermore, the system comprises an implantable energy storage unit 304 and an implantable pump 104, arranged in the device 104.

Referring now to FIG. 51al, the system comprises, externally arranged with regard to the remote unit 140, an implantable energy storage unit 304. Furthermore, the system comprises an implantable electric motor 661 arranged in the device 104. It should be noted that this embodiment does not necessarily comprise an implantable reservoir 107 nor an implantable pump 104. The implantable electric motor 661 is here operatively connected to the body engaging implant 676.

Referring now to FIG. 51am, the system comprises, externally arranged with regard to the remote unit 140, an implantable pump 104. The system further comprises an implantable energy storage unit 304, an implantable electric motor 661, and an implantable reservoir 107, arranged in the device 104.

Referring now to FIG. 51an, the system comprises, externally arranged with regard to the remote unit 140, an implantable energy storage unit 304. The system further comprises an implantable pump 104, an implantable electric motor 661, and an implantable reservoir 107, arranged in the device 104.

Referring now to FIG. 51ao, the system comprises, externally arranged with regard to the remote unit 140, an implantable electric motor 661. The system further comprises an implantable energy storage unit 304, an implantable pump 104, and an implantable reservoir 107, arranged in the device 104.

Referring now to FIG. 51ap, the system comprises, externally arranged with regard to the remote unit 140, an implantable reservoir 107. The system further comprises an implantable energy storage unit 304, an implantable electric motor 661, an implantable pump 104, arranged in the device 104.

Referring now to FIG. 51aq, the system comprises, externally arranged with regard to the remote unit 140, and an implantable electric motor 661. Furthermore, the system comprises an implantable energy storage unit 304 arranged in the device 104. It should be noted that this embodiment does not necessarily comprise an implantable reservoir 107 nor an implantable pump 104. The implantable electric motor 661 is here operatively connected to the body engaging implant 676.

Referring now to FIG. 51ar, an implantable energized medical device which may incorporate one or several of the features described in conjunction with FIGS. 33-51aq, and which may be referred to as a remote unit in other parts of the present disclosure, will be described. The remote unit 140 is configured to be held in position by a tissue portion of a patient. The remote unit 140 comprises a first portion 141′ configured to be placed on a first side of the tissue portion, the first portion 141′ having a first cross-sectional area in a first plane and comprising a first surface configured to face and/or engage a first tissue surface of the first side of the tissue portion. The remote unit 140 further comprises a second portion 141″ configured to be placed on a second side of the tissue portion, the second side opposing the first side, the second portion 141″ having a second cross-sectional area in a second plane and comprising a second surface configured to engage a second tissue surface of the second side of the tissue portion.

The remote unit 140 further comprises a connecting portion 142 configured to be placed through a hole in the tissue portion extending between the first and second sides of the tissue portion. The connecting portion 142 is configured to connect the first portion 141′ to the second portion 141″.

The remote unit 140 here comprises an internal component 685 arranged in the remote unit 140, wherein the internal component 685 may have capabilities of at least one of receiving wireless energy, transmitting wireless energy, receiving communication signals, and transmitting communication signals. The internal component 685, although here illustrated as a single unit, may comprise several units.

The second portion 141″ is here hermetically sealed by means of an outer wall 686 of the second portion comprising a metal, such as titanium. The second portion 141″ may further comprise an internal component 684 having capabilities of at least one of receiving wireless energy, receiving wired energy, receiving communication signals, and transmitting communication signals. Such internal component 684 may comprise an electric motor, a pump, or the like.

An outer wall of the first portion 141′ may comprise or consist of a polymer material.

Accordingly, fluid will likely be able to permeate through the outer wall of the first portion 141′ over time when the remote unit 140 is implanted. In order to protect components of the second portion 141″, a hermetic seal is formed with respect to the connecting portion 142 and with respect to the first portion 141′.

Furthermore, in order to achieve wired communication or energy transfer between the second portion 141″ and the connecting portion 142, the outer wall 686 of the second portion 141″ may comprise a ceramic portion 687 integrated in, or brazed to, the outer wall 686. The ceramic portion 687 may in turn comprise at least one metallic lead 691 travelling through the ceramic portion 687 for transferring electrical energy or information from within the second portion 141″ to an outside of the second portion 141″ and/or from the outside of the second portion 141″, such as from the connecting portion 142, to an inside of the second portion 141″. The outside of the second portion 141″, being outside of the hermetic seal, is commonly referred to as the “wet” side. The at least one metallic lead 691 may in turn be integrated in, or brazed to, the ceramic portion 687, such that the at least one metallic lead 687 can pass the ceramic portion 687 without being further insulated.

Similarly, the connecting portion 142 may comprise an outer wall comprising a metal, such as titanium. Such outer wall of the connecting portion 142 may form a hermetic seal. Furthermore, the outer wall of the connecting portion 142 may comprise a ceramic portion integrated in, or brazed to, the titanium (not shown). At least one metallic lead may travel through the ceramic portion for transferring electrical energy or information from within the connecting portion to an outside of the connecting portion and/or from the outside of the connecting portion to an inside of the connecting portion. The least one metallic lead may be integrated in, or brazed to, the ceramic portion of the connecting portion, such that the at least one metallic lead can pass said ceramic portion without being further insulated.

The at least one metallic lead 691 may connect, or extend to form, an internal lead 698.

Such internal lead 698 may be connected to the internal component 684, as illustrated.

Accordingly, when the connecting portion 142 and the second portion 141″ engage, the at least one metallic lead 691 will engage with a corresponding metallic lead 647 of the connecting portion to form a connection for transferring wired energy and/or wired communication signals. Owing to the integrated ceramic portion 687, the transfer of such wired energy and/or wired communication signals can be achieved through the boundary of the second portion 141″ without breaking the hermetic seal.

Referring now to FIG. 51as, a remote unit 140 similar to the one described in conjunction with FIG. 51ar is illustrated. However, here, no ceramic port is necessary since the internal component 685 located in the first portion 141′ and the internal component 684 located in the second portion 141″ are configured to transmit and/or receive wireless energy and/or wireless communication signals. Thus, the outer wall 686 of the second portion 141″, forming a hermetic seal, need not be penetrated.

Referring now to FIGS. 51at and 51au, an implantable energized medical device which may incorporate one or several of the features described in conjunction with FIGS. 33-51as, and which may be referred to as a remote unit in other parts of the present disclosure, will be described.

The remote unit 140 is configured to be held in position by a tissue portion 610 of a patient. The remote unit 140 comprises a first portion 141′ configured to be placed on a first side of the tissue portion 610, the first portion 141′ having a first cross-sectional area in a first plane and comprising a first surface configured to face and/or engage a first tissue surface of the first side of the tissue portion 610. The remote unit 140 further comprises a second portion 141″ configured to be placed on a second side of the tissue portion 610, the second side opposing the first side, the second portion 141″ having a second cross-sectional area in a second plane and comprising a second surface configured to engage a second tissue surface of the second side of the tissue portion 610. The remote unit 140 further comprises a connecting portion 142 configured to be placed through a hole in the tissue portion 610 extending between the first and second sides of the tissue portion. The connecting portion 142 is configured to connect the first portion 141′ to the second portion 141″.

As illustrated in FIG. 51at, the first portion 141′ is configured to be placed subcutaneously, which is also evident from its implanted position relative to the surface of the skin 699 of the patient.

Furthermore, the first portion may comprise a connecting interface arrangement 641 configured to transfer wired energy and/or wired communication signals and/or fluid to an additional implant in the patient. The connecting interface arrangement 641 is here illustrated as a single unit, it is however to be understood that the connecting interface arrangement 641 may include one or several connecting interface units at different locations on the remote unit 140. In particular, a connecting interface for fluid may require a separate connecting interface unit, or port, and a connecting interface for wired energy or communication signals may require another separate connecting interface unit.

A lead, wire or fluid conduit 643 may form part of the remote unit 140. Such components may also form part of a system which includes the remote unit 140. The lead or wire 643 is configured to connect to the connecting interface arrangement 641 for transferring wired energy and/or wired communication signals. Similarly, the fluid conduit 643 is configured to connect to the connecting interface arrangement 641 for transferring fluid to and from the remote unit 140 and a body engaging implant implanted in another part of the patient's body (not shown), and/or a reservoir implanted in another part of the patient's body (not shown), and/or a pump implanted in another part of the patient's body (not shown).

By flipping the remote unit 140 so that the first portion 140′ and the second portion 141″ switch places, i.e. so that the second portion 141″ is instead located closest to the surface of the skin 699, a wire, lead and/or fluid conduit 643 may run below the tissue portion 610 as opposed to above the tissue portion 610. Whether to run a lead, wire or fluid conduit below the tissue portion 610 or above the tissue portion 610 may be chosen depending on where the lead, wire or fluid conduit shall connect to an additional implant, where such additional implant is located, and/or how such additional implant is implanted. Thus, both the first portion 141′ and the second portion 141″ may be configured to be placed subcutaneously, i.e. closest to the surface of the skin 699 relative to the tissue portion 610, such that the remote unit 140 can be placed with either of the first portion 141′ and the second portion 141″ on side of the tissue portion 610 being closest to the surface of the skin 699.

The connecting interface arrangement 641 may alternatively or additionally be arranged at the second portion 141″, as shown in FIG. 51au. Thus, a lead, wire or fluid conduit may run above the tissue portion 610 even when the first portion portion 141′ is implanted below the tissue portion 610. Similarly, by flipping the remote unit 140 as shown in FIG. 51au, the first portion 141′ will be located above the tissue portion 610 and closest to the surface of the skin 699, and the second portion 141″ will be located below the tissue portion. Thus, a lead, wire or fluid conduit may run below the tissue portion 610 in this configuration.

The terms “above” and “below” in this context shall be understood as directional references where closer to the surface of the skin is “higher” or “above”, and further towards the center of the patient is “lower” or “below”.

A height H1 of the first portion 141′ may be 15 mm or less, such as 10 mm or less, such as 7 mm or less, such as 5 mm or less. The height H1 may be a maximum height of the first portion 141′, i.e. the height H1 may be defined as the height at the location where the first portion 141′ has the largest height. Likewise, a height H2 of the second portion 141″ may be 15 mm or less, such as 10 mm or less, such as 7 mm or less, such as 5 mm or less. The height H2 may be a maximum height of the second portion 141″, i.e. the height H2 may be defined as the height at the location where the first portion 141″ has the largest height.

A length L1 of the first portion 141′ and a length L2 of the second portion 141″ may differ no more than 30%, such as no more than 15%, such as no more than 5%, such as no more than 1%, such as wherein the length L1 of the first portion 141′ and the length L2 of the second portion 141″ are substantially equal, as illustrated.

Similarly, a width (not shown, measured in a direction extending inwards or outwards of the illustrated plane) W1 of the first portion 141′ and a width W2 of the second portion 141″ may differ no more than 30%, such as no more than 15%, such as no more than 5%, such as no more than 1%, such as wherein the width W₁ of the first portion 141′ and the width W2 of the second portion 141″ are substantially equal.

Similarly, a height H1 of the first portion 141′ and a height H2 of the second portion 141″ may differ no more than 30%, such as no more than 15%, such as no more than 5%, such as no more than 1%, such as wherein the height H1 of the first portion 141′ and the height H2 of the second portion 141″ are substantially equal, as illustrated.

With reference to FIG. 52, an embodiment of an implantable remote unit 140, which may be referred to as a remote unit in other parts of the present disclosure, will be described. The remote unit 140 is configured to be held in position by a tissue portion 610 of a patient. The remote unit 140 comprises a first portion 141′ configured to be placed on a first side 612 of the tissue portion 610, the first portion 141′ having a first cross-sectional area in a first plane and comprising a first surface 614 configured to face and/or engage a first tissue surface of the first side 612 of the tissue portion 610. The remote unit 140 further comprises a second portion 141″ configured to be placed on a second side 618 of the tissue portion 610, the second side 618 opposing the first side 612, the second portion 141″ having a second cross-sectional area in a second plane and comprising a second surface 620 configured to engage a second tissue surface of the second side 618 of the tissue portion 610. The remote unit 140 further comprises a connecting portion 142 configured to be placed through a hole in the tissue portion 610 extending between the first and second sides 612, 618 of the tissue portion 610. The connecting portion 142 here has a third cross-sectional area in a third plane. The connecting portion 142 is configured to connect the first portion 141′ to the second portion 141″.

At least one of the first portion and the second portion comprises at least one coil embedded in a ceramic material, the at least one coil being configured for at least one of: receiving energy transmitted wirelessly, transmitting energy wirelessly, receiving wireless communication, and transmitting wireless communication. In the illustrated embodiment, the first portion 141′ comprises a first coil 658 and a second coil 660, and the second portion 141″ comprises a third coil 662. The coils are embedded in a ceramic material 664

As discussed in other part of the present disclosure, the first portion 141′ may comprise a first wireless energy receiver configured to receive energy transmitted wirelessly from an external wireless energy transmitter, and further the first portion 141′ may comprise a first wireless communication receiver. The first wireless energy receiver and the first wireless communication receiver may comprise the first coil. Accordingly, the first coil may be configured to receive energy wirelessly, and/or to receive communication wirelessly.

By the expression “the receiver/transmitter comprising the coil” it is to be understood that said coil may form part of the receiver/transmitter.

The first portion 141′ comprises a distal end 665 and a proximal end 666, here defined with respect to the connecting portion 142. In particular, the proximal end 665 is arranged closer to the connecting portion 142 and closer to the second portion 141″ when the remote unit 140 is assembled. In the illustrated embodiment, the first coil 658 is arranged at the distal end 665.

The first portion 141′ may comprise an internal wireless energy transmitter, and further a first wireless communication transmitter. In some embodiments, the internal wireless energy transmitter and/or the first wireless communication transmitter comprises the first coil 658. However, in some embodiments the internal wireless energy transmitter and/or the first wireless communication transmitter comprises the second coil 660. The second coil 660 is here arranged at the proximal end 665 of the first portion 141′. Such placement of the second coil 660 may provide for that energy and/or communication signals transmitted by the second coil 660 will not be attenuated by internal components of the first portion 141′ when being transmitted to the second portion 141″.

In some embodiments, the first wireless energy receiver and the internal wireless energy transmitter comprises a single coil embedded in a ceramic material. Accordingly, a single coil may be configured for receiving energy wirelessly and for transmitting energy wirelessly. Similarly, the first wireless communication receiver and the first wireless communication transmitter may comprise a single coil embedded in a ceramic material. Even further, in some embodiments a single coil may be configured for receiving and transmitting energy wirelessly, and for receiving and transmitting communication signals wirelessly.

The coils discussed herein are preferably arranged in a plane extending substantially parallel to the tissue portion 610.

The second portion 141″ may comprise a second wireless energy receiver, and/or a second wireless communication receiver. In some embodiments, the third coil 662 in the second portion 141″ comprises the second wireless energy receiver and/or the second wireless communication receiver.

The second portion 141″ comprises a distal end 668 and a proximal end 670, here defined with respect to the connecting portion 142. In particular, the proximal end 668 is arranged closer to the connecting portion 142 and closer to the first portion 141′ when the remote unit 140 is assembled. In the illustrated embodiment, the third coil 662 is arranged at the proximal end 668 of the second portion 141″. Such placement of the third coil 662 may provide for that energy and/or communication signals received by the third coil 662 will not be attenuated by internal components of the second portion 141″ when being received from the first portion 141′.

The first portion 141′ may comprise a first controller 300a connected to the first coil 658, second coil 660, and/or third coil 662. The second portion 141″ may comprise a second controller 300b connected to the first coil, 658, second coil 660, and/or third coil 662.

In the illustrated embodiment, the first portion 141′ comprises a first energy storage unit 304a connected to the first wireless energy receiver 308a, i.e. the first coil 658. The second portion comprises a second energy storage unit 304b connected to the second wireless energy receiver 308b, i.e. the third coil 662. Such an energy storage unit may be a solid-state battery, such as a thionyl-chloride battery.

In some embodiments, the first coil 658 is configured to receive energy transmitted wirelessly by the external wireless energy transmitter and store the received energy in the first energy storage unit 304a. Furthermore, the first coil 658 and/or the second coil 660 may be configured to wirelessly transmit energy stored in the first energy storage unit 304a to the third coil 662, and the third coil 662 may be configured to receive energy transmitted wirelessly by the first coil 658 and/or the second coil 660 and store the received energy in the second energy storage unit 305b.

The first energy storage unit 304a may be configured to store less energy than the second energy storage unit 304b, and/or configured to be charged faster than the second energy storage unit 304b. Hereby, charging of the first energy storage unit 304a may be relatively quick, whereas transfer of energy from the first energy storage unit 304a to the second energy storage unit 304b may be relatively slow. Thus, a user can quickly charge the first energy storage unit 304a, and will not during such charging be restricted for a long period of time by being connected to an external wireless energy transmitter, e.g. at a particular location. After having charged the first energy storage unit 304a, the user may move freely while energy slowly transfers from the first energy storage unit 304a to the second energy storage unit 304b, via the first and/or second coil and the third coil.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is configured to receive electromagnetic waves at a frequency above a frequency level, and/or to transmit electromagnetic waves at a frequency below the frequency level, wherein the second portion is configured to receive and/or transmit electromagnetic waves at a frequency below the frequency level, and wherein the frequency level is 100 kHz.

In some embodiments, wherein the first portion is configured to transmit electromagnetic waves at the frequency below the frequency level to the second portion.

In some embodiments, the first portion is configured to transmit electromagnetic waves at the frequency above the frequency level to an external device.

In some embodiments, the frequency level is 40 kHz or 20 kHz.

In some embodiments, the electromagnetic waves comprise wireless energy and/or wireless communication.

In some embodiments, the first portion comprises a first wireless energy receiver for receiving energy transmitted wirelessly by an external wireless energy transmitter above the frequency level, and an internal wireless energy transmitter configured to transmit energy wirelessly to the second portion below the frequency level, and the second portion comprises a second wireless energy receiver configured to receive energy transmitted wirelessly by the internal wireless energy transmitter below the frequency level.

In some embodiments, the first portion comprises a first controller comprising at least one processing unit.

In some embodiments, the second portion comprises a second controller comprising at least one processing unit.

In some embodiments, the first controller is connected to a first wireless communication receiver in the first portion for receiving wireless communication from an external device above the frequency level, the first controller is connected to a first wireless communication transmitter in the first portion for transmitting wireless communication to a second wireless communication receiver in the second portion below the frequency level.

In some embodiments, the second controller is connected to the second wireless communication receiver for receiving wireless communication from the first portion below the frequency level.

In some embodiments, the first portion comprises an outer casing made from a polymer material.

In some embodiments, the outer casing forms a complete enclosure, such that electromagnetic waves received and transmitted by the first portion must travel through the casing.

In some embodiments, the second portion comprises an outer casing made from titanium.

In some embodiments, the outer casing forms a complete enclosure, such that electromagnetic waves received and transmitted by the second portion must travel through the casing.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is configured to receive and/or transmit electromagnetic waves at a frequency below the frequency level, and wherein the frequency level is 100 kHz.

In some embodiments, the second portion is configured to receive and/or transmit electromagnetic waves at a frequency below the frequency level.

In some embodiments, the first portion is configured to transmit electromagnetic waves at the frequency below the frequency level to the second portion.

In some embodiments, the first portion is configured to transmit electromagnetic waves at the frequency below the frequency level to an external device.

In some embodiments, the frequency level is 40 kHz or 20 kHz.

In some embodiments, the electromagnetic waves comprise wireless energy and/or wireless communication.

In some embodiments, the first portion comprises a first wireless energy receiver for receiving energy transmitted wirelessly by an external wireless energy transmitter below the frequency level, and an internal wireless energy transmitter configured to transmit energy wirelessly to the second portion below the frequency level, and the second portion comprises a second wireless energy receiver configured to receive energy transmitted wirelessly by the internal wireless energy transmitter below the frequency level.

In some embodiments, the first portion comprises a first controller comprising at least one processing unit.

In some embodiments, the second portion comprises a second controller comprising at least one processing unit.

In some embodiments, the first controller is connected to a first wireless communication receiver in the first portion for receiving wireless communication from an external device below the frequency level, the first controller is connected to a first wireless communication transmitter in the first portion for transmitting wireless communication to a second wireless communication receiver in the second portion below the frequency level.

In some embodiments, the second controller is connected to the second wireless communication receiver for receiving wireless communication from the first portion below the frequency level.

In some embodiments, the first portion comprises an outer casing made from a polymer material.

In some embodiments, the first portion comprises an outer casing made from titanium.

In some embodiments, the outer casing forms a complete enclosure, such that electromagnetic waves received and transmitted by the first portion must travel through the casing.

In some embodiments, the second portion comprises an outer casing made from titanium.

In some embodiments, the outer casing forms a complete enclosure, such that electromagnetic waves received and transmitted by the second portion must travel through the casing.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is made from a polymer material, the second portion comprises a casing made from titanium, wherein the casing forms a complete enclosure.

In some embodiments, the casing of the second portion forms a complete enclosure such that the entirety of the outer surface of the second portion is covered by the casing, when the second portion is connected to the connecting portion.

In some embodiments, the first portion comprises a casing made from the polymer material.

In some embodiments, the casing of the first portion forms a complete enclosure such that the entirety of the outer surface of the first portion is covered by the casing.

In some embodiments, the connecting portion comprises a connection arranged to connect to the first and second portion respectively and carry electrical signals and/or energy.

In some embodiments, the connection is arranged in a core of the connecting portion such that it is encapsulated by outer material of the connecting portion.

In some embodiments, the connecting portion comprises a ceramic material.

In some embodiments, the connection is encapsulated within the ceramic material.

In some embodiments, the first portion comprises a first connection configured to connect to the connection of the connecting portion.

In some embodiments, the second portion comprises a second connection configured to connect to the connection of the connection portion.

In some embodiments, the casing of the second portion is hermetically sealed.

In some embodiments, the second connection is arranged such that the hermetical seal of the second portion is kept intact.

In some embodiments, the casing of the first portion is hermetically sealed.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, and wherein the connecting portion is configured to extend between the first portion and the second portion along a central extension axis, and wherein the second portion is configured to extend in a length direction being divergent with the central extension axis, and wherein the connecting portion has a substantially constant cross-sectional area along the central extension axis, or wherein the connecting portion has a decreasing cross-sectional area in a direction from the first portion towards the second portion along the central extension axis, and/or wherein the second portion has a substantially constant cross-sectional area along the length direction, or wherein the second portion has a decreasing cross-sectional area in the length direction.

In some embodiments, the third cross-sectional area is smaller than the first cross-sectional area.

In some embodiments, the connecting portion is tapered in the direction from the first portion towards the second portion along the central extension axis.

In some embodiments, the connecting portion has a circular or oval cross-section along the central extension axis with a decreasing diameter in the direction from the first portion towards the second portion.

In some embodiments, the second portion is tapered in the length direction.

In some embodiments, the connecting portion has a circular or oval cross-section in the length direction with a decreasing diameter in the length direction.

In some embodiments, the length direction extends from an interface between the connecting portion and the second portion towards an end of the second portion.

In some embodiments, the length direction extends in a direction substantially perpendicular to the central extension axis.

FIGS. 53a and 53b illustrate a gear arrangement and magnetic coupling for coupling the remote unit to an implant exerting force on a body part, and in particular a gear arrangement for transferring mechanical movement through an outer housing of the device or an outer housing of the second portion 141″.

The housing 484 of the device or second portion 141″ may be present in some embodiments of the device. In such embodiments, the housing 484 is configured to enclose, at least, the controller (not shown), motor M, any receivers and transmitters if present (not shown), and any gear arrangements G, Gi, G2 if present. Hereby, such features are protected from bodily fluids. The housing 484 may be an enclosure made from one of or a combination of: a carbon-based material (such as graphite, silicon carbide, or a carbon fiber material), a boron material, a polymer material (such as silicone, Peek®, polyurethane, UHWPE or PTFE), a metallic material (such as titanium, stainless steel, tantalum, platinum, niobium or aluminum), a ceramic material (such as zirconium dioxide, aluminum oxide or tungsten carbide) or glass. In any instance the enclosure should be made from a material with low permeability, such that migration of fluid through the walls of the enclosure is prevented.

The remote unit may comprise at least part of a magnetic coupling, such as a magnetic coupling part 490a. A complementary part of the magnetic coupling, such as magnetic coupling part 490b, may be arranged adjacent to the remote unit 140, so as to magnetically couple to the magnetic coupling part 490a and form the magnetic coupling. The magnetic coupling part 490b may form part of an entity not forming part of the remote unit 140. However, in some embodiments the second portion 141″ comprises several chambers being hermetically sealed from each other. Such chambers may be coupled via a magnetic coupling as discussed herein. The magnetic coupling 490a, 490b provide for that mechanical work output by the remote unit 140 via e.g. an electric motor can be transferred from the device to an implantable medical device for stretching the stomach wall of the patient. In other words, the magnetic coupling 490a, 490b provides for that mechanical force can be transferred through the housing 484.

The coupling between components, such as between a motor and gear arrangement, or between a gear arrangement and a magnetic coupling, may be achieved by e.g. a shaft or the like.

In some embodiments, for example as illustrated in FIG. 53a, a force output of a motor MO in the second portion 141″ is connected to the magnetic coupling part 490a. The magnetic coupling part 490a transfers the force output from the motor MO to the magnetic coupling part 490b, i.e. via the magnetic coupling 490a, 490b. The force output transferred via the magnetic coupling 490a, 490b here has a torque T1, which is substantially the same torque as delivered by the motor MO. The magnetic coupling part 490b is connected to a gear arrangement G, located external to the device, for example in a medical implant configured to exert force on a body part, or intermediate to a medical implant configured to exert force on a body part. The gear arrangement G is configured to increase the torque of the force delivered via the magnetic coupling 490a, 490b to deliver a force with torque T2 being higher than torque T1 to a medical implant. Consequently, low torque may be provided by the motor MO, i.e. a relatively small force with high angular speed, which is transferred via the magnetic coupling 490a, 490b before the torque is increased via gear arrangement G to achieve a relatively large force with low angular speed. Hereby, the magnetic coupling 490a, 490b may utilize relatively weak magnetic forces to transfer the mechanical work through the housing 484 of the device without the risk of slipping between the magnetic coupling parts 490a, 490b.

In some embodiments, for example as illustrated in FIG. 53b, a force output of a motor MO in the second portion 141″ is connected to a first gear arrangement G1, which in turn is coupled to the magnetic coupling part 490a. The motor MO here provides a mechanical force with torque TO. The magnetic coupling part 490a transfers the force output from the motor MO to the first gear arrangement G1. The first gear arrangement G1 is configured to increase the torque of the force delivered from the motor MO to deliver a force with a higher torque T1 to the magnetic coupling 490a, 490b. The magnetic coupling part 490a transfers the force with torque T1 to the magnetic coupling part 490b. The magnetic coupling part 490b is connected to a second gear arrangement G2, located external to the device, for example in a medical implant configured to exert force on a body part, or intermediate to a medical implant configured to exert force on a body part. The second gear arrangement G2 is configured to increase the torque of the force delivered via the magnetic coupling 490a, 490b to deliver a force with torque T2 being higher than torque T1, and thus higher than torque TO, to a medical implant. Consequently, low torque may be provided by the motor MO, i.e. a relatively small force with high angular speed. The torque of the force provided by the motor MO is then increased by the first gear arrangement G1, before the force is transferred via the magnetic coupling 490a, 490b. The torque of the force transferred via the magnetic coupling 490a, 490b is then yet again increased via the second gear arrangement G2 to achieve a relatively large force with low angular speed. Hereby, the magnetic coupling 490a, 490b may utilize relatively weak magnetic forces to transfer the mechanical work through the housing 484 of the device without the risk of slipping between the magnetic coupling parts 490a, 490b. Furthermore, since some of the torque increase is made within the second portion 141″, and a remaining portion of the torque increase is made external to the device and the second portion 141″, the gear arrangements G1, G2 may be sized and configured appropriately to share the work of increasing the torque.

FIG. 53c schematically illustrates an energy storage 304b connected to a wireless energy transmitter 308. The energy storage 304b and the wireless energy transmitter 308 are arranged in one portion or chamber of the second portion 141″. Furthermore, a wireless energy receiver 308e is arranged in another portion or chamber of the second portion 141″. The portions or chambers may be separated or defined by respective housings, external walls and/or internal walls 484a, 484b. The wireless energy transmitter 308d is configured to wirelessly transmit energy to the wireless energy receiver 308e. Hereby, an internal energy transfer is achieved within the second portion 141″. The wireless energy transmitter 308d and wireless energy receiver 308e may comprise one or more coils, respectively. The wireless energy receiver 308e may be connected to a further energy storage 680 arranged within the second portion 141″. Such energy storage 680 may be connected to a medical implant, such that the energy storage 680 can deliver energy to the medical implant. In some embodiments however, the wireless energy receiver 308e is directly connected to a medical implant to deliver energy directly to the medical implant, thus omitting the energy storage 680.

The magnetic coupling schematically described with reference to FIG. 53a may be combined with a hydraulic pump, such as any one of the hydraulic pumps described with reference to FIGS. 54a-58, such that the motor and optionally a gear system may be completely enclosed in a housing, such as in the remote unit, and only in magnetic connection with the hydraulic pump, such that the hydraulic pump can be propelled by an encapsulated electrical motor. This reduces the risk that electronics of the implantable system is placed in connection with bodily fluids and also reduces the risk electronic or electrical components are placed in connection with the hydraulic fluids used in the hydraulic pump.

FIG. 54a shows an embodiment of a hydraulic pump 104 which may be used as a part of a hydraulic operation device for operating any of the hydraulic embodiments of the medical device disclosed herein. The hydraulic pump 104 may be placed in, or in direct connection with a main portion of a medical device, or may be placed in a remote unit, for example replacing part of the hydraulic operation device placed in the remote unit described with reference to FIG. 15. In the embodiment of FIG. 54a, the hydraulic pump 104 is a peristaltic hydraulic pump shown in cross-section. The implantable peristaltic pump 104 comprises a deflectable hollow member 401 for fluid transportation, in form of a tubing made from a resilient material, such as an elastomeric polymer material, such as silicone, Parylene® coated silicone, NBR, Hypalon, Viton, PVC, EPDM, Polyurethane or Natural Rubber. The deflectable hollow member 401 is placed between a first portion of a fluid conduit 109′ at the inlet of the hydraulic pump 104 and a second portion of a fluid conduit 109″ at the outlet of the hydraulic pump 104. The deflectable hollow member 401 is adapted to be deflected by operable compression members 402 or “wipers”, adapted to engage and compress the hollow member 401, and thus transport the hydraulic fluid. The compression member 402 is propelled by the motor MO via a gear system G. The hollow member 401 is placed inside a peristaltic pump housing 403, such that the hollow member 401 is compressed between the operable compression member 402 and the housing 403. The peristaltic pump 104 is a sealed pump which means that fluid will not leak through the pump even at standstill. As the peristaltic pump is a sealed pump no additional valve is needed to keep the fluid through the fluid conduits 109′, 109″ closed.

The deflectable hollow member 401 is connected to or integrated with fluid conduits 109′109″, which in turn are connectable with fluid conduits (109) of the hydraulic operation devices in any of the embodiments described herein. When the compression member 402 is propelled in a counterclockwise direction, it creates a peristaltic wave which presses hydraulic fluid through the hollow member 401 and further through the second portion of the fluid conduit 109″. When the compression member 402 is propelled in a clockwise direction, it creates a peristaltic wave which presses hydraulic fluid through the hollow member 401 and further through the first fluid conduit 109′. By using two peristaltic pumps 104 of the embodiment of FIG. 54a, in connection with the medical device according to the embodiment described with reference to FIG. 15, the third member (101c) can be operated in a first and second direction by operating the motor MO of the first peristaltic pump in a first and second direction, and the fourth member (101d) can be operated by operating the motor MO of the second peristaltic pump in a first and second direction.

FIG. 54b shows the peristaltic pump in accordance with the embodiment of FIG. 54a in a side view in which the electrical motor MO and gear system G for propelling the compression member 402 is shown. The electrical motor MO is adapted to transform electrical energy to mechanical work. The electrical motor MO may receive electrical energy from a receiving unit receiving wireless energy transmitted from an energy transmitting unit external to the body of the patient, and/or may receive electrical energy stored in an implantable energy storage unit. The electrical motor MO is in the embodiment of FIGS. 54a and 54b a brush-less direct current electrical motor MO, but in alternative embodiment the electrical motor could be an electrical motor MO selected from an alternating current (AC), a linear electrical motor, an axial electrical motor, a piezo-electric motor, a multiple phase motor, such as a three-phase motor, a bimetal motor, and a memory metal motor.

The force output of the electrical motor MO is in connection with a force input of a gear system G adapted to receive mechanical work having a first force and first speed, and output mechanical work having a different second force and a different second speed, such that the high speed movement supplied by the electrical motor MO is transformed to low speed movement with increased force.

The gear system G may for example comprise a gear system having the configuration such as the gear system G described with reference to FIGS. 55a and 55b. In alternative embodiments, it is conceivable that the gear system G comprises a transmission system of some other configuration, such as a conventional gear wheel system, a worm gear system or a belt transmission system.

FIG. 55a shows an embodiment of an implantable gear system G which may be used as a transmission for any of the electrical motors shown herein, for the purpose of transforming the high speed movement supplied by the electrical motor to low speed movement with increased force. The implantable gear system G of FIG. 55a is adapted to receive mechanical work having a first force and first speed, and output mechanical work having a second, different force and a second different speed. The gear system G comprises a force input 442 connected to an operable element 443′ adapted to engage a first gear 444 having the shape of a hollow cylinder, comprising a first number of teeth 444t, for example 160, on the peripheral outside thereof, and a second gear 445 having the shape of a hollow cylinder, comprising a greater number of teeth 445t than the first gear, for example 462, on the inside surface thereof. The operable element 443′ is adapted to engage the inside 444a of the first gear 444, such that the outside 444b of the first gear 444 is pressed against the inside 445a of the second gear 445 such that the teeth 444t of the first gear 444 are interengaged with the teeth 445t of the second gear 445 in position P₁ interspaced by positions (for example the position P₂) at which the teeth are not interengaged. The operation of the operable element 443′ advances the position P₁ and thereby causes relative rotation between the first gear 444 and the second gear 445. In the embodiment shown in FIG. 55a, the second gear 445 comprises two more teeth 445t than the first gear 444, resulting in the first gear 444 rotating 2/160 or 1/80 of a revolution for each revolution that the operable element 443′ performs, which results in a transmission of 80 times, i.e. the force output (449 of FIG. 55b) provides a force with 1/80 of the speed and 80 times the force, thus increasing the force which can be exerted on the stomach wall by the electrical motor, 80 times. In the embodiment shown in FIG. 55a the operable element 443′ slides radially against the inner surface of the first gear 444. For reducing the friction a lubricating fluid may be present in the gear system G, it is further conceivable that the operable element 443′ or the surface against which the operable implant 443′ slides may comprise a self-lubricating material, such as Graphalloy, Nyliol or PTFE.

FIG. 55b shows the gear system G in a sectional side view, in an embodiment in which the gear system G comprises a third gear 446 having an inside 446a comprising the same amount of teeth 446t as the outside 444b of the first gear 444. The teeth 446t of the third gear 446 are adapted to interengage with the teeth of the first gear 444 such that the third gear 446 rotates in relation to the second gear 445, along with the interengaged position (P₁ of FIG. 55a). The third gear 446 is in connection with a force output 449 of the gear system 440 by means of a radially extending connecting structure 447 for transferring force from the third gear 446 to the force output 449.

The gear system G of FIGS. 55a and 55b could for example be made of a metallic material, plastic material or ceramic material. In one embodiment, the gear system is made from non-metallic and/or non-magnetic material, such that the gear system G does not affect the energy transfer to an implantable energy receiver. The gear system G may be lubricated with a biocompatible lubricant, such as hyaluronic acid, and may, for that purpose, be placed inside a reservoir adapted to hold a hydraulic fluid, which also may serve as a lubricant. The gear system G may be encapsulated by an enclosure for preventing bodily fluids from affecting the gear system G and/or the in-growth of human tissue in the gear system and/or the leakage of hydraulic and/or lubricating fluids. The enclosure may be a non-metallic and/or non-magnetic enclosure, such that the material of the enclosure does not affect the ability of transferring wireless energy to a wireless energy receiver of the operable implant. The gear system may be encapsulated separately or may be encapsulated along with an electrical motor (such as shown in FIGS. 54a,54b) or alongside additional components (such as shown in FIGS. 56a and 57a).

FIG. 56 shows a cross-sectional view of an electrical motor MO in combination with a gear system G for propulsion of a hydraulic pump 104. The hydraulic pump described with reference to FIG. 56 may be used as a part of a hydraulic operation device for operating any of the hydraulic embodiments of the medical device disclosed herein. The hydraulic pump 104 may be placed in, or in direct connection with a main portion of a medical device, or may be placed in a remote unit, for example replacing part of the hydraulic operation device placed in the remote unit described with reference to FIG. 15. The electrical motor MO is connected to the controller 300 which in turn is connected to an energy storage unit 40. The energy storage unit 40 may be a battery, a chargeable battery or a capacitor by means of which energy can be stored in the body of the patient.

The controller 300, the energy storage unit 40 and the motor MO and gear system G the may be enclosed by a housing 484 such that the controller 300 is protected from bodily fluids. The housing 484 may be an enclosure made from one of or a combination of: a carbon based material (such as graphite, silicon carbide, or a carbon fiber material), a boron material, a polymer material (such as silicone, Peek®, polyurethane, UHWPE or PTFE), a metallic material (such as titanium, stainless steel, tantalum, platinum, niobium or aluminum), a ceramic material (such as zirconium dioxide, aluminum oxide or tungsten carbide) or glass. In any instance the enclosure should be made from a material with low permeability, such that migration of fluid through the walls of the enclosure is prevented.

Turning now to the hydraulic pump 104 shown in FIG. 56. In the embodiment shown in FIG. 56, the force output 449 of the gear system G is threaded 449t and engages a correspondingly threaded portion 451t of the movable wall 451 such that the rotating force created by the motor MO and gear system G is transferred to a linear force moving the movable wall 451. The threaded force output 449 is enclosed by pleated bellows portions 452 both above and below the movable wall 451 such that the threaded force output 449 is protected from the fluid in the lumens of the reservoirs 107a, 107b. The reservoirs 107a, 107b has a common moveable wall 451 for changing the volume of the implantable fluid reservoirs 107a, 107b and thereby increasing fluid in the first fluid reservoir 107a simultaneously with decreasing fluid in the second fluid reservoir 107b and vice versa. The peristaltic pump is a sealed pump which means that fluid will not leak through the pump even at standstill. As the peristaltic pump is a sealed pump no additional valve is needed to keep the fluid through the fluid conduits 109′, 109″ closed.

The movable wall pump 104 of FIG. 56 is a sealed pump which means that fluid will not leak through the pump even at standstill. As the movable wall pump 104 is a sealed pump, no additional valve is needed to keep the fluid through the fluid conduits 109′, 109″ closed.

FIG. 57a shows a cross-sectional view of a hydraulic pump comprising two expandible reservoirs 107a, 107b. The hydraulic pump described with reference to FIG. 57a may be used as a part of a hydraulic operation device for operating any of the hydraulic embodiments of the medical device disclosed herein. The hydraulic pump 104 may be placed in, or in direct connection with a main portion of a medical device, or may be placed in a remote unit, for example replacing part of the hydraulic operation device placed in the remote unit described with reference to FIG. 15. The hydraulic pump 104 of FIG. 57a comprises an encapsulated motor MO, gear system G, controller 300 and energy storage unit 40 being identical to that described with reference to FIG. 56. Turning to the hydraulic pump 104, the force output 449 is, in the embodiment described in FIG. 57a a hollow shaft equipped with inner threads (not shown) adapted to engage outer threads 453t of a threaded member 453, such that the interaction between the hollow shaft 449 and the threaded member 453 transforms the radially rotating force generated by the motor MO and the gear system G, to a linear force. The threaded member 453 is connected to a radially extending engaging member 454 adapted to engage the first and second reservoirs 107a, 107b containing a hydraulic fluid. The reservoirs 107a, 107b may be fixated to the radially extending engaging members 454, for example by means of an adhesive, such that the reservoirs 107a, 107b are forced to expand when the radially extending engaging member 454 is moved upwards in the expanding direction of the reservoirs 107a, 107b. The first reservoir 107a is connected to a first fluid conduit and the second reservoir 107b is connected to a second fluid conduit 109″. The embodiment shown in FIG. 57a further comprises a pleated bellows portions 452 for encapsulating and protecting the force output 449 and the threaded member 453 from bodily fluids. The reservoirs 107a, 107b are preferably made from medical grade implantable silicone or Parylene® coated medical grade implantable silicone, but may in alternative embodiments be made from another resilient material such as NBR, Hypalon, Viton, PVC, EPDM, Polyurethane or Natural Rubber. When the reservoirs 107a, 107b are compressed and expanded they function as hydraulic pumps for moving hydraulic fluid any of the hydraulic embodiments herein. By using two pumps 104 of the embodiment of FIG. 56 or the embodiment of FIG. 57a, in connection with the medical device according to the embodiment described with reference to FIG. 15, the third member (101c) can be operated in a first and second direction by operating the motor MO of the first pump in a first and second direction, and the fourth member (101d) can be operated by operating the motor MO of the second pump in a first and second direction.

FIG. 57b shows a cross-sectional view of a hydraulic pump 104 similar to the hydraulic pump or the embodiment of FIG. 57a. The hydraulic pump described with reference to FIG. 57b may be used as a part of a hydraulic operation device for operating any of the hydraulic embodiments of the medical device disclosed herein. The hydraulic pump 104 may be placed in, or in direct connection with a main portion of a medical device, or may be placed in a remote unit, for example replacing part of the hydraulic operation device placed in the remote unit described with reference to FIG. 15. In the embodiment of FIG. 57b, the hydraulic pump 104 comprises one expandible reservoir 107. The hydraulic pump 104 comprises an encapsulated motor MO, gear system G, controller 300 and energy storage unit 40. The motor MO is configured to generate force in a radial direction by rotation of the force output in the form of a shaft 481. The shaft 481 is equipped with outer threads 481t adapted to engage inner threads 483t of a compression member 483, such that the interaction between the threaded shaft 481, 481t and the threaded portion 483t of the compression member 483 transforms the radially rotating force generated by the motor MO and the gear system G, to a linear force acting in the axial direction of the shaft 481, and thus makes up a transmission T. The axial force acts on the compression member 483 which engages a first resilient wall 102a of the compressible reservoir 107 for compressing the compressible reservoir 107 and thus increasing the pressure on a hydraulic fluid in the compressible reservoir 107. The compression member 483 may be fixated to the first resilient wall portion 102a by means of an adhesive, such that the reservoir 107 is forced to expand when the compression member 483 moves in the expanding direction of the reservoir 107. The reservoir 107 is connected to a fluid conduit (not shown) for conducting hydraulic fluid from the compressible reservoir to the and from the reservoir 107. The reservoir 107 is preferably made from medical grade implantable silicone or Parylene® coated medical grade implantable silicone, but may in alternative embodiments be made from another resilient material such as NBR, Hypalon, Viton, PVC, EPDM, Polyurethane or Natural Rubber. When the reservoir 107 is compressed and expanded it functions as hydraulic pump for moving hydraulic fluid in any of the hydraulic embodiments herein.

The hydraulic pump 104 further comprises at least one bearing 482 for the shaft 481 placed between the gear system G and the compressible reservoir 107. The bearing 482 is configured to withhold at least half of the force in the axial direction, for reducing the axial load on the motor MO and the gear system G which is caused by the compression of the reservoir 107. In the embodiment shown in FIG. 57b, the bearing 482 is a ball bearing, but in other embodiments the bearing may comprise a roller bearing or a plain bearing preferably including a self-lubricating material such as PTFE or HDPE.

The gear system G is connected to the motor MO, and placed between the motor MO and transmission T and adapted to receive mechanical work via the shaft 481 having a force and a speed, and output mechanical work having a stronger force and a lower speed. The compressible reservoir 107 comprises a first resilient wall portion 102a and a second resilient wall portion 102b, wherein the first resilient wall portion 102a is more resilient than the second resilient wall portion 102b.

In alternative embodiments, the compression member 483 may be directly connected to the first resilient wall portion 102a, and in such embodiments, the threaded portion 483t may be integrated in the first resilient wall portion 102a.

In the embodiment shown in FIG. 57b, the hydraulic pump 104 further comprises a pressure sensor 106 connected to the compressible reservoir 107 and configured to sense the pressure in the compressible reservoir 107. The pressure sensor 106 is integrated in, and placed on the outside of, the second resilient wall portion 102b of the compressible reservoir 107. The pressure sensor 106 comprises a strain gauge-based pressure sensor 106 such as for example described with reference to FIGS. 59a-59e. It may be important to measure strain or pressure in or exerted by the medical device, as too high strain or pressure risks hampering the blood flow to the tissue of the stomach wall, which in the long term could lead to damage of the tissue and in the worst-case lead to necrosis.

The compressible reservoir 107 in the embodiment shown in FIG. 57b comprises a first and second resilient wall portion 102a, 102b in the form of a first and second circular diaphragm 102a, 102b.

The first resilient wall portion 102a has a convex shape facing the compression member 483, and the second resilient wall portion 102b has a convex shape facing away from the compression member 483 and a lumen is formed between the two diaphragms 102a, 102b, and being enclosed by the concave surfaces of the diaphragms 102a, 102b. The first resilient wall portion 102a is configured to be compressed and thus inverted, such that the part of the first resilient wall portion 102a facing the compression member 483 assumes a concave shape facing the compression member 483, and as such, a convex shape is formed towards the lumen of the compressible reservoir 107. The inverted, convex, portion of the first resilient wall portion 102a thus enters the concave shape of the second resilient wall portion 102b. The portion of the compression member 483 configured to engage the first resilient wall portion 102a comprises a convex portion for facilitating the inversion of the convex portion of the first resilient wall portion 102a. In the embodiment shown in FIG. 57b, the first resilient wall portion 102a is more resilient than the second resilient wall portion 102b such that the compressible reservoir 107 can create a suction when the compression member 483 moves in the direction away from the compressible reservoir 107 thus enabling the compressible reservoir 107 to expand. In the embodiment shown in FIG. 57b, a major portion of the first resilient wall portion is made from a material having a modulus of elasticity (E) which is less than 70% or the modulus of elasticity (E) of the material of a major portion of the second resilient wall portion 102b. In alternative embodiments, it is conceivable that the first and second resilient wall portions 102a, 102b are made from the same material, but with the second resilient wall portion 102b being more than 1.5 times as thick as the first resilient wall portion 102a. In the embodiment shown in FIG. 57b, the two diaphragms 102a, 102b are pressed against each other, for creating the sealed lumen between the first and second diaphragm, by means of a fixation ring 485, which is screwed into the housing 484.

In the embodiment shown in FIG. 57b, the hydraulic pump further comprises a shaft sealing 486, which is a sealing engaging the shaft and thus creating a seal between the portion of the pump housing 484 comprising the motor MO, gear system G, energy storage unit 40 and controller 300, and the portion of the pump housing 484 comprising the compressible reservoir 107. The seal reduces the risk that hydraulic fluid that may leak from the compressible reservoir 107 will come in contact with any of the motor MO, gear system G, energy storage unit 40 and/or controller 300. In the embodiment shown in FIG. 57b, the shaft sealing comprises a spring-loaded PTFE sealing 486. A spring engages the housing 484 of the hydraulic pump 104 and the PTFE sealing for creating a constant elastic pressure between the sealing and the shaft 481 which ensures a self-lubricating tight seal. In alternative embodiments, the spring may be replaced by a different type of elastic element, such as an elastic element made from an elastomer. In alternative embodiment, the shaft sealing 486 could be a shaft sealing made from another self-lubricating material such as HDPE.

The hydraulic pump 104 of FIG. 57b is enclosed by a pump housing 484, which in the embodiment shown in FIG. 57b is a titanium housing 484. In alternative embodiments, the housing could be made from another medical grade metal alloy, such as medical grade stainless steel or could comprise a ceramic material such as zirconium carbide, or a stiff medical grade polymer material such as Ultra-high-molecular-weight polyethylene (UHMWPE) or Polytetrafluoroethylene (PTFE) or a thermoplastic polyester such as polylactide (PLA). The housing could also comprise at least one composite material, such as any combination of metallic/ceramic and polymer materials or a polymer material reinforced with organic or inorganic fibers, such as carbon or mineral fibers.

FIG. 57c shows a cross-sectional view of a hydraulic pump 104 similar to the hydraulic pump of the embodiment of FIG. 57b. In the embodiment of FIG. 57c, the hydraulic pump comprises one expandible reservoir 107. The hydraulic pump 104 comprises a housing 484 comprising a first and a second chamber C1, C2 separated from each other by a barrier 484′. Just as in the embodiment of FIG. 57c, the first chamber C1 comprises the motor MO configured for transforming electrical energy to mechanical work and the gear system gear system G adapted to receive mechanical work having a first force and first speed, and output mechanical work having a different second force and a different second speed, such that the high speed movement supplied by the electrical motor MO is transformed to low speed movement with increased force. The output mechanical work having the different second force and different second speed acts on a shaft 481 which transfers the force to a magnetic coupling 490a, 490b for transferring mechanical work from the motor MO to an actuator in the form of a compression member 483 for compressing the expandible reservoir 107 for pressing a hydraulic fluid through the conduit 109a. The magnetic coupling 490a, 490b comprises a first disc shaped member 490a mounted to the shaft 481 such that the first disc shaped member 490a rotates along with the shaft 481. The shaft 481 is supported by ball bearings 482 assisting in the centering of the shaft 481.

The first disc shaped member 490a comprises magnets (or a material susceptible to magnetic fields) 491 evenly distributed axially in a circular formation on the distal surface of the first disc shaped member 490a.

The barrier 484′ separates the first chamber C1 of the housing 484 from the second chamber C2 of the housing. In the embodiment shown in FIG. 57c, the barrier 484′ is made from the same material as the outer wall of the housing 484, i.e. medical grade titanium. In the embodiment shown in FIG. 57c the barrier is materially integrated with the portion of the outer wall of the housing 484 enclosing the second chamber C2. However, in other embodiments it is equally conceivable that the barrier is materially integrated with the portion of the outer wall of the housing 484 enclosing the first chamber C1. In any event, the purpose is the both the first and second chambers C2 should be hermetically enclosed and separated from each other.

The second part of the magnetic coupling comprises a second disc shaped member 490b positioned in the second chamber C2 and held in place by a ball bearing 482b being fixated to the inside of the wall of the housing 484 enclosing the second chamber C2 by means of an internal wall portion 498. The second disc shaped member 490b comprises magnets (or a material susceptible to magnetic fields) 491b evenly distributed in a circular formation axially on the distal surface of the first disc shaped member 490b. The magnets 490b of the second disc shaped member 490b are configured to be magnetically connected to the magnets 491a of the first disc shaped member 490a such that the second disc shaped member 490b is dragged by the first disc shaped member 490a by means of the magnetic connection. As such, force from the motor MO is transferred from the first hermetically enclosed chamber C1 to the second hermetically enclosed chamber C2.

The second disc shaped member 490b comprises a threaded shaft which is configured to be placed in and engage with a sleeve of a compression member 483. The sleeve of the compression member 483 comprises inside threads 483t for creating a transmission T that transforms the radially rotating force generated by the motor MO and the gear system G, to a linear force acting in the axial direction of the shaft 481, and thus makes up a transmission T.

The compression member 483 is a disc shaped element having a distal surface engaging a first resilient wall portion 102a of the reservoir 107 for moving the first resilient wall portion 102a and thereby compressing the reservoir 107. The periphery of the compression member 483 comprises a flange 483f extending towards the first chamber C1 in the proximal direction creating a lateral surface area towards the housing 484. The lateral surface of the flange 483f is configured to engage the first resilient wall portion 102a for creating a rolling crease of the first resilient wall portion 102a. The disc shaped compression member 483 is rigid and made from titanium, just as the rest of the housing 484.

That the compression member 483 is rigid makes the reservoir 107 stiff.

The reservoir 107 is further enclosed by a second wall portion 102b which is a rigid titanium wall portion through which the conduit 109a enters the reservoir 107. Compression of the reservoir 107 thus forces the fluid from the reservoir through the conduit 109a. The housing 484 further comprises a transfer channel 478 creating a fluid connection between the second chamber C2 and a portion of the second chamber C2′ placed more distally. The transfer channel ensures that the pressure is the same in the second chamber C2 and distal portion of the second chamber C2′. The distal portion C2′ of the second chamber C2 comprises an expansion portion comprising a resilient membrane 495 configured to move to alter the volume of the distal portion C2′ of the second chamber C2 for compensating for the changes to the volume of the reservoir 107 which is created by the movement of the first resilient wall portion 102a of the reservoir 107. As such, the pressure in the second chamber C2 will be substantially constant. The resilient membrane 495 is in the embodiment shown in FIG. 57c made from a medical grade elastic silicone material but may in alternative embodiments be made from another biocompatible polymer material, such as polyurethane.

The hydraulic pump of FIG. 57c further comprises a pressure sensor 106 placed on the first resilient wall portion 102a of the chamber 107 for sensing the pressure in the chamber 107. The sensor 106, which may be a pressure sensor of the types described with reference to FIGS. 59a-59e, is connected to electrical leads 474 for transferring an electrical sensor signal from the pressure sensor 106 to the controller 300. The electrical leads 474 passes from the second chamber C2 to the first chamber C1 through an electrically insulating ceramic grommet 494 integrated in the barrier 484′ wall such that the leads 474 can pass the barrier 484′ without being further insulated which enables the leads 474 to pass through the barrier 484′ whilst the barrier hermetically separates the first chamber C1 from the second chamber C2. It may be important to measure strain or pressure in or exerted by the medical device, as too high strain or pressure risks hampering the blood flow to the tissue of the stomach wall, which in the long term could lead to damage of the tissue and in the worst-case lead to necrosis.

A first portion 109a of the fluid conduit is connected to an implantable hydraulic force transfer device 496 comprising a first chamber V1 configured to house a first fluid, and as such the first portion 109a of the fluid conduit forms a fluid inlet into the first chamber V1. The first chamber V1 is in connection with a movable wall portion 497 for varying the size of the first chamber V1. The movable wall portion 497 is in turn connected to a second chamber V2 configured to house a second fluid. The second chamber comprises an outlet formed by a second portion 109b of the fluid conduit. The second portion 109b of the fluid conduit fluidly connects the second chamber C2 to a conduit (109) in any of the hydraulic embodiments described herein. As such, the implantable hydraulic force transfer device 496 transfers hydraulic force from a remote unit to the main portion of a medical device without mixing the first and second fluids.

In the embodiment shown in FIG. 57c, the implantable hydraulic force transfer device 496 comprises a cylinder-shaped housing in which the piston-like movable wall portion 497 moves linearly.

The piston-like movable wall portion 497 seals against the inner side of the wall of the cylinder-shaped housing such that the first and second chambers V1, V2 remains separated. The implantable hydraulic force transfer device 496 enables the system to have a first fluid in the compressible reservoir 107 and in the first chamber V1 of the implantable hydraulic force transfer device 496. This part of the system may be hermetically sealed in such a way that leakage is highly improbable, which enables this part of the system to use a fluid which cannot be allowed to escape into the body, such as an oil-based fluid, such as a silicone oil. The second part of the system, comprising the second chamber C2 of the implantable hydraulic force transfer device 496, the second portion 109b of the fluid conduit, and the rest of the hydraulic operation device of the medical device (not shown) will have a second fluid which must be a biocompatible fluid as some level of leakage or diffusion may be hard to avoid. In the second part of the system the fluid could for example be an isotone aqueous fluid, such as a saline solution.

In the embodiment shown in FIGS. 57a-57h, the housing 484 and the housing of the implantable hydraulic force transfer device 496 is a titanium housing. However, it is equally conceivable that the housing is made from another biocompatible material such as a medical grade metal alloy, such as medical grade stainless steel or a ceramic material such as zirconium carbide, or a stiff medical grade polymer material such as Ultra-high-molecular-weight polyethylene (UHMWPE) or Polytetrafluoroethylene (PTFE) or a thermoplastic polyester such as polylactide (PLA).

In alternative embodiments, the magnetic coupling described with reference to FIGS. 57c and 57d could be used in connection with another type of pumps, such as the pumps described with reference to FIGS. 54a, 54b, 56 and 58. In the alternative, the magnetic coupling could be used in connection with a gear pump. It is also conceivable that the magnetic coupling could be used in connection with a mechanical actuator configured to transfer mechanical force from the magnetic coupling to a medical device to exert a force on a body portion of a patient. The mechanical actuator could be an actuator configured to transfer a rotating force into a linear force, such as the transmission (T) described with reference to FIGS. 56-57h.

FIG. 57d shows a hydraulic pump in an embodiment similar to the embodiment shown in FIG. 57c. One difference with the embodiment of FIG. 57d in comparison to the embodiment of FIG. 57c is that the first coupling part 490a′ comprises magnets 491a′ or material susceptible to magnetic fields which are placed radially along an outer periphery, on the lateral surface, of the cylinder-like first coupling part 490a′. The magnets 491a′ of the first coupling part 490a′ are magnetically connected to magnets 491b′ placed radially on the inner letteral surface of the cylinder-shaped second coupling part 490b′. The magnets 491a′,491b′ of the first and second coupling parts 490a′, 490b′ are separated from each other by the barrier 484′. The second coupling part 490b′ is connected to a rotatable shaft which is supported by ball bearings 482b being fixated to the inside of the wall of the housing 484 enclosing the second chamber C2 by means of an internal wall portion 498. The rotatable shaft comprises a threaded portion which is configured to be placed in and engage with a sleeve of a compression member 483. The sleeve of the compression member 483 comprises inside threads 483t for creating a transmission T that transforms the radially rotating force generated by the motor MO and the gear system G, to a linear force acting in the axial direction of the shaft 481, and thus makes up a transmission T.

Another difference between the embodiment shown in FIG. 57c and the embodiment shown in FIG. 57d is in the implantable hydraulic force transfer device 496. In the embodiment shown in FIG. 57d, the implantable hydraulic force transfer device 496 comprises a movable wall portion 497′ in the form of a bellows with a pleated flexible wall portion which can be compressed and expanded. The material of the flexible wall portion could be an elastic material, such as an elastic polymer material or a substantially inelastic material such as a metal material forming a metal bellows which is mainly flexible due to its shape. In an alternative embodiment, the flexible wall portion can be purely elastic and thus be without the pleats, which means that the expansion and contraction of the reservoir is done purely based on the elasticity of the material in the flexible wall. The flexible movable wall portion 497′ encloses the first chamber V1 and keeps the chamber V1 completely separated from the chamber V2. The implantable hydraulic force transfer device 496 enables the system to have a first fluid in the compressible reservoir 107 and in the first chamber V1 of the implantable hydraulic force transfer device 496. This part of the system may be hermetically sealed in such a way that leakage is highly improbable, which enables this part of the system to use a fluid which cannot be allowed to escape into the body, such as an oil-based fluid, such as a silicone oil. The second part of the system, comprising the second chamber C2 of the implantable hydraulic force transfer device 496, the second portion 109b of the fluid conduit, and the hydraulic components of the implantable medical device (not shown) will have a second fluid which must be a biocompatible fluid as some level of leakage or diffusion may be hard to avoid. In the second part of the system the fluid could for example be an isotone aqueous fluid, such as a saline solution.

FIG. 57e shows an embodiment of a hydraulic pump 104 which is similar to the embodiment shown in FIG. 57b. One difference in comparison to the embodiment of FIG. 57b is that the compression member 483 has a flat circular surface engaging the first resilient wall portion 102a of the reservoir 107.

The flat surface is bonded to the first resilient wall portion 102a such that the first resilient wall portion 102a moves along with the compression member 483. The compression member 483 has a diameter such that a distance 483d is created between the compression member 483 and the portion of the housing facing the compression member 483. The distance is slightly more than two times the thickness of the first resilient wall portion 102a, such that the first resilient wall portion 102a can be folded such that a rolling crease of the first resilient wall portion 102a is created which moves along with the compression member 483. The distance 483d is smaller than the radius (or half cross-sectional distance) of the compression member 483. The distance is 483d is also smaller than half the radius of the compression member 483. The first resilient wall portion 102a, towards the second chamber C2, being either folded or supported by the compression member means that ensures that the reservoir 107 will be substantially stiff which enables the fluid amount in the hydraulically operable medical device connected to the reservoir 107 to remain the same even as the pressure exerted on the hydraulically operable medical device increases.

The embodiment of FIG. 57e differs from the embodiment of FIGS. 57c and 57d in that it only comprises a single chamber C1. The housing 484 of the hydraulic pump 104 of FIG. 57e comprises an expansion portion placed in the proximal portion of the hydraulic pump 104 (on the right side of the hydraulic pump of FIG. 57e). The expansion portion comprises a first and second resilient membrane 495a, 495b with a silicone oil filling the space formed between the first and second resilient membranes 495a, 495b. The oil between the first and second resilient membrane 495a, 495b reduces the risk of diffusion of fluids through the expansion portion. The first and second resilient membranes 495a, 495b are placed on two sides of a portion 484″ of the housing comprising a hole through which the fluid can travel as the expansion portion compensates for the changes to the volume of the reservoir 107 which is created by the movement of the first resilient wall portion 102a of the reservoir 107. As such, the pressure in the first chamber C1 will be substantially constant. The first and second resilient membranes 495a, 495b are in the embodiment shown in FIG. 57e made from a medical grade elastic silicone material but may in alternative embodiments be made from another biocompatible polymer material, such as polyurethane.

Another aspect of having the housings of any of the embodiments herein, is that the atmospheric pressure that the patient exists in may vary. At sea level, the air pressure is about 101 kPa, in a commercial airplane at cruising altitude, the air pressure is about 80 kPa which is about the same as in Mexico city, whereas in La Paz, the highest situated city, air pressure is only 62 kPa. This difference in air pressure affects any gaseous fluid, such as the air present in the chamber C1 in the embodiment of FIG. 57e. The reduced atmospheric air pressure means that the gaseous fluid inside of the housing needs to be able to expand if the pressure in the housing should remain the same. If the pressure in the housing would increase 20%-40%, the motor would have to operate the hydraulic medical device against that pressure which would mean that the motor would have to be more powerful which would require more energy. As the expansion portion comprises a resilient membrane, the expansion portion allows the gaseous fluid in the housing to expand which at least reduces the pressure increase in the housing in response to a reduced atmospheric pressure.

FIG. 57f differs from the embodiment of FIG. 57e only in that the chamber C1 is completely filled with a liquid dielectric silicone oil. The liquid fluid could in the alternative be a synthetic single-phase liquid dielectric fluid, such as ElectroCool EC-100, from Engineered Fluids, or a 2-phase coolant such as Fluorinert or Novec from 3M. The fluid in the chamber C1 is non-conductive and as such does not risk damaging the electrical components placed in the chamber C1, such as the energy storage unit 40. In the embodiment shown in FIG. 57f, the expandible reservoir 107, the conduit 109 and the medical device configured to exert force on the body portion of the patient forms the second chamber and second hydraulic system configured to comprise a second liquid which is a hydraulic liquid configured to transfer force. The second liquid may be an isotone aqueous liquid, such as a saline solution.

In the embodiment shown in FIG. 57f, the first chamber comprises the motor MO, the gear system G and the transmission T for transforming the rotating force generated by the motor MO to a linear force for pressing on the expandible reservoir 107. Advantages with having the housing and the first chamber C1 entirely filled with a liquid fluid includes the liquid acting as a cooling agent for components that may produce heat, such as the controller 300, the energy storage unit 40, the motor MO, gear system G, bearing 482 and transmission T, and as a lubricant for components that may require lubrication, such as the motor MO, gear system G, bearing 482 and transmission T.

Just as in FIG. 57e, the housing 484 of the hydraulic pump 104 comprises an expansion portion 495a, 484″, 495b placed in the proximal portion of the hydraulic pump 104 (on the right side of the hydraulic pump of FIG. 57f), such that the housing can expand when the expandable reservoir 107 expands.

In alternative embodiments, the liquid filled first chamber C1 could be used in connection with another type of pump, i.e. the shaft 481 could be connected to another type of pump, such as the pumps described with reference to FIGS. 54a,54b,56 and 58, or a gear pump.

FIG. 57g shows an embodiment of a hydraulic pump 104 which is similar to the embodiment shown in FIG. 57d. The main difference with the embodiment shown in FIG. 57g is that it made more compact as the gear system is integrated in the magnetic coupling. The magnetic coupling thus comprises a magnetic gear which transfers a week force with a high speed into a stronger force with lower speed.

The magnetic coupling/gear comprises a first coupling part 490a′ fixated to the shaft 481 connected to the electrical motor MO such that the first coupling part 490a′ rotates along with the electrical motor MO. The first coupling part 490a′ comprises a first number of magnets 491a′, which in the embodiment shown in FIG. 57g is 6 magnets, 3 with each polarity (3 pole pairs). The magnets are placed radially along an outer periphery, on the lateral surface, of the cylinder-like first coupling part 490a′. The second coupling part 490b′ comprises a second number of magnets 491b′, placed radially on the inner letteral surface of the cylinder-shaped second coupling part 490b′. In the embodiment shown in FIG. 57g the second coupling part 490b′ comprises 26 magnets, 13 with each polarity. Between the first coupling part 490a′ and the second coupling part 490b′ there is a stationary part, which is a portion of the barrier 484′.

The stationary part comprises a plurality of intermediate ferromagnetic elements 499 thus placed between the first and second coupling parts 490a′, 490b′. The intermediate ferromagnetic elements 499 directs the concentration of the magnetic lines between the magnets 491a′, 491b′ of the first coupling part 490a′ and the second coupling part 490b′. The gear ratio between the first coupling part 490a′ and the second coupling part 490b′ is the number of magnetic pole pairs on the second coupling part 490a′ divided by the number of magnetic pole pairs on the second coupling part 490b′. In the embodiment shown in FIG. 57g, the gear ratio is 13/3. The number of intermediate ferromagnetic elements 499 is equal to the sum of pole pairs on the first and second coupling parts 490a′, 490b′. In the embodiment shown in FIG. 57g this means that the number of intermediate ferromagnetic elements 499 is 16 (13+3). In operation, this set up of magnetic gear changes the direction of rotation of the coupling, which means that that in operation the second coupling part 490b′ will rotate in the opposite direction and 4,33 times slower than the first coupling part 490a′. The embodiment having a magnetic gear have a number of advantages, for example, the magnetic gear is quiet, does not wear and does not need to be lubricated. In alternative embodiments it is conceivable that the magnetic gear is used in combination with a traditional gear wheel gear-system or a transmission of the kind described with reference to FIGS. 55a, 55b.

The second coupling part 490b′ is connected to a rotatable shaft which is supported by roller bearings 482 being fixated to the inside of the wall of the housing 484. The rotatable shaft comprises a threaded portion which is configured to be placed in and engage with a sleeve of a compression member 483. The sleeve of the compression member 483 comprises inside threads 483t for creating a transmission T that transforms the radially rotating force generated by the motor MO and the gear system G, to a linear force acting in the axial direction of the shaft 481, and thus makes up a transmission T.

FIG. 57h shows an embodiment of a hydraulic pump 104 which is similar to the embodiment shown in FIG. 57g. The main difference with the embodiment shown in FIG. 57h is that the expansion portion is replaced with two resilient reservoirs 107′, 107″ which are placed in indentations in the housing, on respective two opposite sides of the housing. The two resilient reservoirs 107′, 107″ are configured to expand and contract to compensate for the changes to the volume of the reservoir 107 which is created by the movement of the first resilient wall portion 102a of the reservoir 107. As such, the pressure in the second chamber C2 will be substantially constant. The two resilient reservoirs 107′, 107″ are made from a medical grade elastic silicone material but may in alternative embodiments be made from another biocompatible polymer material, such as polyurethane.

FIG. 57I shows and embodiment of a system comprising a motor MO, gear system G and two implantable pumps 460′,460″. The implantable pumps 40′,460″ described with reference to FIG. 57I may be used as a part of a hydraulic operation device for operating any of the hydraulic embodiments of the medical device disclosed herein. The hydraulic pump 104 may be placed in, or in direct connection with a main portion of a medical device, or may be placed in a remote unit, for example replacing part of the hydraulic operation device placed in the remote unit described with reference to FIG. 15. In the embodiment shown in FIG. 57I, the force output of the motor MO is connected to a force input of the gear system G. The gear system G is configured to reduce the speed and increase the force of the movement generated by the motor MO, such that the movement exiting the gear system G at the force output of the gear system G is a mechanical force with a lower speed and a greater force than the movement entering the force input of the gear system G. Typically, an implantable brushless DC motor, such as the motors provided by Maxon group or Dr. Fritz Faulhaber, typically produces a rotational speed exceeding 10 000 rpm. For such a motor to be able to mechanically operate any of the hydraulic pumps described herein, a gear system G is needed. In the embodiment shown with reference to FIG. 57I, the gear system G reduces the rotational speed 100 times, to about 100 rpm. The force output of the gear system G is mechanically connected to a common rotating shaft 463. The first hydraulic pump comprises a first gerotor pump 460′ and the second hydraulic pump comprises a second gerotor pump 460″. The common rotating shaft 463 is mechanically connected to an inner rotor 461′ of the first gerotor pump 460′ and an inner rotor 461″ of the second gerotor pump, such that the motor MO propels the first and second gerotor pump 460′, 460″. A gerotor is a positive displacement pump comprising consists of an inner rotor 461 and an outer rotor 462. The inner rotor 461 has 6 teeth, while the outer rotor has 7 teeth (the importance being that the outer rotor 462 has one tooth more than the inner rotor 461. The axis of the inner rotor 461, which is the rotational center of the common rotating shaft 463, is offset from the rotational center or axis of the outer rotor 462. Both the inner and outer rotors 461, 462 rotate on their respective axes. The geometry of the two rotors 461, 462 partitions the volume between them into 6 different dynamically changing volumes. During the rotation cycle, each of these volumes changes continuously, so any given volume first increases, and then decreases. An increase creates a vacuum. This vacuum creates suction, and hence, this part of the cycle is where the inlet 109′ is located. As a volume decreases compression occurs which pumps the fluid though the outlet 109″.

In the embodiment shown in FIG. 57I, the first gerotor pump 460′ is configured to be in fluid connection with a first portion of a hydraulic operation device for pumping hydraulic fluid into the first hydraulic operation device. The second gerotor pump 460″ is configured to be in fluid connection with a second hydraulic operation device for pumping hydraulic fluid into the second hydraulic operation device. The inlets 109′, 109″ of the first and second gerotor pumps 460′,460″ are configured to be connected to a reservoir for holding hydraulic fluid, or in the alternative, the first inlet 109′ is configured to be connected to a first implantable reservoir and the second inlet 109″ is configured to be connected to a second implantable reservoir.

In alternative embodiments, the first and second hydraulic pump mechanically connected to a common rotating shaft could be pump comprising at least one compressible hydraulic reservoir (such as the pump described with reference to FIG. 57a), a pump comprising a displaceable wall (such as the pump described with reference to FIG. 56), or a peristaltic pump (such as the pump described with reference to FIGS. 54a and 54b).

The embodiment of two pumps mechanically connected to a common rotating shaft, described with reference to FIG. 57I, could be implemented in any of the embodiments disclosed herein in which there are more than one operable hydraulic members, such as in embodiment disclosed with reference to FIG. 15.

The operation device shown as 100 in the embodiments described with reference to FIGS. 1A—4A, 4C—6, 16—21 and 60 could comprise a piezo-electric operation device, such as a piezo-electric motor. In the same way, the motors shown as MO in FIGS. 5—7, 9, 11, 12A, 13A, 14A, 14B, 14C, 14D, 15, 17B′, 31F-31H, 31Y′-32, 35A-35C, 51AA, 54B, 56-58, could be piezoelectric motors. And the pumps shown in FIGS. 17B, 18B, 19B, 20B, 51AB-51AE, 51AH-51AK, 51AM-51AP, 53A-53B, 54A-54B could be operated by means of piezo-electric operation devices, such as piezo-electric motors.

The piezoelectric effect is a property of certain solid materials to generate an electrical voltage in response to an applied mechanical stress (so-called direct piezoelectric effect) and to deform elastically in response to an applied electrical voltage (so-called inverse piezoelectric effect). The piezoelectric effect is a reversible process, meaning that materials exhibiting the direct piezoelectric effect also exhibit the inverse piezoelectric effect.

Materials exhibiting the piezoelectric effect are denoted as piezoelectric materials. Examples of piezoelectric materials comprise: crystalline materials, such as lithium niobate, lithium tantalate and quartz; ceramics, such as lead zirconate titanate, potassium niobate and barium titanate; polymers, such as polyvinylidene fluoride.

Piezoelectric coefficients are a fundamental property of piezoelectric materials. A given piezoelectric material is characterized by a set of piezoelectric coefficients, wherein a piezoelectric coefficient is a measure of the relationship between the applied mechanical stress along a first direction and the generated electric charge along a second direction. Piezoelectric coefficients are usually expressed in units of picocoulombs per newton (pC/N). The value of piezoelectric coefficients may strongly vary depending on the piezoelectric material and piezoelectric coefficient being considered. For example, the d_33 piezoelectric coefficient is commonly reported for piezoelectric materials and quantifies the electric charge generated along a given direction in response to the mechanical stress applied along the same direction.

A piezoelectric motor or piezo motor is a type of electric motor that uses the inverse piezoelectric effect to generate mechanical motion, typically linear or rotatory motion. Piezo motors are often used in applications where precise positioning and fine control of movement are required. Piezo motors have the advantage of providing high motion accuracy, being possible to miniaturize and being relatively immune to interference, such as electromagnetic interference. Piezoelectric motors can also be manufactured without magnetic and/or metallic parts, and instead be manufactured from ceramics or certain composites. This feature is particularly advantageous in medical and biotechnology applications with strong magnetic fields. Piezoelectric motors can thus be made MRI-safe, meaning that the patient can undergo Magnetic Resonance Imaging (MRI) while having the piezo motor implanted. MR1 is a medical imaging technique used to form pictures of the anatomy and the physiological processes of the body using strong magnetic fields. Conventional implantable electromagnetic motors prevent the use of MR1 as the strong magnetic field risks damaging both the patient and the implant.

Also, compared to classical electromagnetic motors, piezo motors may have a simpler structure and smaller footprint. Piezo motors may offer improved positioning accuracy and simpler design, as linear motion may be obtained directly, without the need of mechanical coupling elements otherwise required to convert the rotary motion of classical electromagnetic motors to linear motion.

That linear motion can be obtained directly may improve the positioning accuracy.

An additional advantage of piezoelectric motors is that they usually feature higher energy-efficiency and less power consumption compared to conventional electromagnetic motors.

As discussed in more detail in the sections below, inchworm motors, inertial motors, walk-drive and ultrasonic motors are three four common types of piezoelectric motors.

FIG. 58A shows an embodiment of an inchworm motor MO configured to generate linear motion. The inchworm motor MO comprises a first lateral piezoelectric actuator 801a and second lateral piezoelectric actuator 801b. The first lateral piezoelectric actuator 801a is laterally connected to a first clutching actuator 802a′ and a second clutching actuator 802a″. The second lateral piezoelectric actuator 801b is laterally connected to a third clutching actuator 802b′ and a fourth clutching actuator 802b″. The inchworm motor MO is configured to impart a linear motion to a movable member 805. The movable member 805 is configured to be attached to the load or mechanism to be moved.

FIG. 58B illustrates an operation cycle of the piezoelectric inchworm motor MO wherein the movable member 805 is linearly moved in a direction to the right in the illustration by sequentially controlling the first and second piezoelectric actuators 801a, 801b and the clutching actuators 802a′, 802a″, 802b′, 802b″. In an initial relaxation state, the movable member 805 is detached from all the clutching actuators 802a′, 802a″, 802b′, 802b″. The inchworm motor MO is subsequently brought into an initialization state by electrically activating the second and fourth clutching actuators 802a″, 802b″. As a result, the clutching actuators 802a″, 802b″ extend and clutch the movable member 805.

In step 1, the first and second lateral actuators 801a, 801b extend in response to an applied electrical voltage. As a result, the movable member 805 undergoes a first linear displacement with a distance equal to half the distance of the extension of the lateral actuators 801a, 801b.

In step 2, the first and third clutching actuators 802a′, 802b′ are electrically activated. As a result, the first and third clutching actuators 802a′, 802b′ extend and clutch the movable member 805.

In step 3, the electrical voltage applied to the third and fourth clutching actuators 802a″, 802b″ is decreased as compared to the initialization state. As a result, the third and fourth clutching actuators 802a″, 802b″ detach from the movable member 805.

In step 4, the electrical voltage applied to the first and second lateral actuators 801a, 801b is decreased as compared to step 1. As a result, the first and second lateral actuators 801a, 801b contract and the movable member 805 undergoes a second linear displacement with a distance equal to half the distance of the contraction of the lateral actuators 801a, 801b.

In step 5, the second and fourth clutching actuators 802a″, 802b″ are electrically activated such that they extend and clutch the movable member 805.

In step 6, the electrical voltage applied to the first and second clutching actuators 802a′, 802b′ is decreased as compared to step 2. As a result, the first and second clutching actuators 802a′, 802b′ contract and detach from the movable member 805.

The steps from 1 to 6 may be repeated a number of times in the sequence illustrated above in order to move the movable member 805 by a desired distance.

This configuration of an inchworm piezoelectric motor MO creating a linear motion could for example be used to generate the linear motion needed for the operation of the flexible shafts (135) in the embodiments described with reference to FIGS. 11, 12A-12E, 13A-14D′, 28A—28C, 30A-31E, 31I, 31K′, 31M-31T, 32, 33 thus replacing the combination of rotational electrical motors (MO) and transmission elements transferring the rotating force to a linear force.

This configuration of an inchworm piezoelectric motor MO creating a linear motion could for example be used to generate the linear motion needed for the direct operation of the members in 15-17B, 17C—21, 29A—29C, 31L-31L′, 31U-31U″, thus replacing the hydraulic cylinder creating the linear motion.

In the embodiment shown in FIG. 58B, the inchworm motor is configured to generate a linear motion at a speed in the range 1 mm/s to 10 mm/s, a stroke length in the range 4 mm-30 mm and a force in the range 2 N-30 N.

In alternative embodiments, the movable member 805 may be replaced with a rotary module (not shown) such that the inchworm motor can be configured to generate rotary motion. In such embodiments, the rotating inchworm motor could be used as the rotational electrical motor described with reference to FIGS. 5, 6, 7, 8, 9, 11-15, 17B′, 31D-31H-31K, 31Y-35, 51AA, 54B and 56-57I.

An inchworm motor configured to generate rotary motion may have a rotational speed in the range 0,5 mrad/s to around 70 mrad/s and a torque ranging from around 100 Nmm to around 900 Nmm.

FIG. 58C illustrates an embodiment of a piezoelectric inertial motor MO configured to generate linear motion. The motor MO comprises a movable member 805, a piezoelectric actuator 801, a recoiling member 804 and a base 806. The movable member 805 is attached to the piezoelectric actuator 801. The piezoelectric actuator 801 is attached to the recoiling member 804. The movable member 805 is in contact with the base 806. The movable member 805 is configured to be attached to the load or mechanism to be moved.

An operation mode of the piezoelectric inertial motor MO described in FIG. 58C comprises two steps. In step 1, the piezoelectric actuator 801 slowly elongates in response to an applied voltage. Due to the static friction between the movable member 805 and the base 806, the movable member 805 does not move. In step 2, the piezoelectric actuator 801 rapidly contracts in response to a change in the applied voltage. As a result of the recoil produced by the recoiling member 804, the movable member 805 moves by a distance Ax to the left in the illustration of FIG. 58C.

By reversing the operation cycle, the piezoelectric inertial motor MO generates motion in the opposite direction.

In the embodiment shown in FIG. 58C, the piezoelectric inertial motor MO is configured to generate linear motion and may feature a speed in the range 1 mm/s to 10 mm/s, a stroke length in the range 4 mm-30 mm and a force in the range 2 N-30 N.

This configuration of a piezoelectric inertial motor MO creating a linear motion could for example be used to generate the linear motion needed for the operation of the flexible shafts (135) in the embodiments described with reference to FIGS. 11, 12A-12E, 13A-14D′, 28A—28C, 30A-31E, 31I, 31K′, 31M-31T, 32, 33 thus replacing the combination of rotational electrical motors (MO) and transmission elements transferring the rotating force to a linear force.

This configuration of a piezoelectric inertial motor MO creating a linear motion could for example be used to generate the linear motion needed for the direct operation of the members in 15-17B, 17C—21, 29A—29C, 31L-31L′, 31U-31U″, thus replacing the hydraulic cylinder creating the linear motion.

In alternative embodiments of piezoelectric inertial motors (not shown), the movable member 805 may be replaced with a with a rotary module such that the piezoelectric inertial motor is configured to generate rotary motion. Piezoelectric inertial motors configured to generate rotary motion may have a rotational speed in the range 1 mrad/s-100 mrad/s and a torque in the range 100 Nmm-900 Nmm. In such embodiments, the rotating piezoelectric inertial motor could be used as the rotational electrical motor described with reference to FIGS. 5, 6, 7, 8, 9, 11-15, 17B′, 31D-31H-31K, 31Y-35, 51AA, 54B and 56-57I.

Yet another design of a piezoelectric motor suitable for use in the implantable medical device described herein is the walk-drive motor. Walk-drive motors take their name from the fact that their working principle essentially resembles a walk. Linear motion is achieved through the coordinated and sequential action of a number of piezoelectric actuators acting as legs.

FIG. 58D shows an embodiment of a piezoelectric walk-drive motor MO. The piezoelectric walk-drive motor MO comprises a number of piezoelectric actuators 801a-801d attached to a supporting member 800. The piezoelectric actuators 801a-801d may be divided into a first set 801a, 801c and a second set 801b, 801d. The first and second set 801a, 801c and 801b, 801d of piezoelectric actuators may be controlled independently. The piezoelectric walk-drive motor MO is configured to impart a linear motion to a movable member 805 by sequentially controlling the piezoelectric actuators 801a-801d. The movable member 805 is configured to be attached to the load or mechanism to be moved.

A piezoelectric walk-drive motor 805 may be operated in various operation modes, each offering specific advantages in terms of performance.

FIG. 58E illustrates an operation cycle of a piezoelectric walk-drive motor MO according to one embodiment. In this embodiment, the first set of piezoelectric actuators 801a, 801c is controlled by a first electrical voltage V_1. The second set of piezoelectric actuators 801b, 801d is controlled by a second electrical voltage V_2. The cycle comprises a number of steps.

In step 1, in response to a change in V_1, the first set of piezoelectric actuators 801a, 801c stretch and make contact with the movable member 805. When in contact, the first set of piezoelectric actuators 801a, 801c are bended sideways in a direction opposite to the motion direction D. Conversely, the second set of piezoelectric actuators 801b, 801d detach from the movable member 805 in response to a change in V_2.

In step 2, the first set of piezoelectric actuators 801a, 801c maintain contact with the movable member 805 and bend in the motion direction D in response to a change in V_1. The second set of piezoelectric actuators 801b, 801d remain detached from the movable member 805. As a result of the friction between the first set of piezoelectric actuators 801a, 801c and the movable member 805, the movable member 805 is moved in the motion direction D.

In step 3, in response to a change in V_2, the second set of piezoelectric actuators 801b, 801d stretch and make contact with the movable member 805. When in contact, the second set of piezoelectric actuators 801b, 801d are bended in a direction opposite to the motion direction D. Conversely, the first set of piezoelectric actuators 801a, 801c detach from the movable member 805 in response to a change in V_1.

In step 4, the second set of piezoelectric actuators 801b, 801d maintain contact with the movable member 805 and bend in the motion direction D in response to a change in V_2. The first set of piezoelectric actuators 801a, 801c remain detached from the movable member 805. As a result of the friction between the second set of piezoelectric actuators 801b, 801d and the movable member 805, the movable member 805 is moved in the motion direction D.

The piezoelectric actuators 801a-801d in FIG. 58D and FIG. 58E may be bimorph piezoelectric actuators. A bimorph actuator comprises at least two piezoelectric layers bonded together and oppositely responding to a given applied voltage. E.g., one layer extends and the other contracts. As a result, the bimorph actuator may stretch and bend. Alternatively, any of piezoelectric actuators 801a-801d in FIG. 58D and FIG. 58E may comprise a top part (illustrated as 801a′ in FIG. 58D), configured to be attached to the supporting member 800, and a bottom part (exemplified by illustration as 801a″ in FIG. 58D). The top part 801a′ is configured to deform perpendicularly to the motion direction D in response to the voltage applied to the piezoelectric actuator 801a. The bottom part 801a″ is configured to deform parallelly to the motion direction D in response to the voltage. As a result, the piezoelectric actuator 801a may stretch and bend.

By reversing the operation cycle, the piezoelectric walk-drive motor MO generates motion in the opposite direction.

In the embodiment shown in FIGS. 58D and 58E, the piezoelectric walk-drive motor MO is configured to generate linear motion at a speed in the range 1 mm/s to 10 mm/s and a force in the range 2 N-30 N. As the maximum stroke is limited by the length of the movable member 805 (also called a runner), there is no set limit for the maximum stroke.

This configuration of a piezoelectric walk-drive motor MO creating a linear motion could for example be used to generate the linear motion needed for the operation of the flexible shafts (135) in the embodiments described with reference to FIGS. 11, 12A-12E, 13A-14D′, 28A—28C, 30A-31E, 31I, 31K′, 31M-31T, 32, 33 thus replacing the combination of rotational electrical motors (MO) and transmission elements transferring the rotating force to a linear force.

This configuration of a piezoelectric walk-drive motor MO creating a linear motion could for example be used to generate the linear motion needed for the direct operation of the members in 15-17B, 17C—21, 29A—29C, 31L-31L′, 31U-31U″, thus replacing the hydraulic cylinder creating the linear motion.

In alternative embodiments of the piezoelectric walk-drive motor (not shown), the movable member 805 may be replaced with a with a rotary module such that the piezoelectric walk-drive motor is configured to generate rotary motion. Piezoelectric walk-drive motors configured to generate rotary motion may have a rotational speed in the range 0,5 mrad/s to around 70 mrad/s and a torque ranging from around 100 Nmm to around 900 Nmm. In such embodiments, the rotating piezoelectric walk-drive motor could be used as the rotational electrical motor described with reference to FIGS. 5, 6, 7, 8, 9, 11-15, 17B′, 31D-31H-31K, 31Y-35, 51AA, 54B and 56-57I.

An ultrasonic motor is another type of piezoelectric motor. In ultrasonic motors, a first component of the motor, the stator, supports mechanical vibrations in the ultrasonic frequency range—from tens to hundreds of kHz. The stator comprises a number of piezoelectric actuators. Ultrasonic mechanical vibrations are excited in the stator in response to an electrical voltage applied to the piezoelectric actuators. The stator is configured to transfer the ultrasonic vibrations to a second component of the motor, such as a rotor or slider depending on the scheme of operation. Depending on the scheme of operation, various types of motion, such as linear or rotary, may be imparted to the second component.

A rotary ultrasonic motor is a piezoelectric ultrasonic motor configured to generate rotary motion. Rotary ultrasonic motors comprise traveling wave ultrasonic motors (TWUSM) and standing wave ultrasonic motor (SWUSM). In TWUSMs the stator vibrates according to a travelling wave pattern.

In SWUSMs the stator vibrates according to a standing wave pattern.

FIG. 57F shows an embodiment of a TWUSM MO. The TWUSM MO comprises a ring-shaped stator 810 with a top and a bottom surface. The stator 810 is configured to engage with a ring-shaped rotor 811. The stator 810 comprises a ring-shaped member 810′, a first number of piezoelectric actuators 801a and a second number of piezoelectric actuators 801b. The piezoelectric actuators 801a, 801b are attached to the ring-shaped member 810′. An alternating electrical voltage V_A may be applied from a first voltage generator 812a (typically a controller connected to an energy source) to the piezoelectric actuators 801a. An alternating electrical voltage V_B, phase-shifted with respect to VA, may be applied to the from a second voltage generator 812b to the second number of piezoelectric actuators 801b. The ring-shaped member 810′ may comprise a number of teeth 813. The rotor 811 is configured to be attached to the load or mechanism to be moved.

The first number of piezoelectric actuators 801a deform in response to the voltage V_A such that they induce a first vibration pattern in the stator 810. The second number of piezoelectric actuators 801b deform in response to the voltage V_B such that they induce a second vibration pattern in the stator 810. The interference of the first and second vibration pattern excites a travelling wave 814 in the stator 810. The travelling wave 814 has a given propagation direction D_1, either clock-wise or counter-clockwise. The regions of maximum displacement—so-called antinodes—and regions of no displacement—so-called nodes—of the travelling wave pattern oscillate transversely with respect to the top and bottom surface of the stator 810, but they also travel circumferentially along the stator 810 perimeter.

The propagation of the travelling wave 814 makes the stator vibrate accordingly. As a result, the stator 810 imparts a rotatory motion to the rotor 811 in a rotation direction D_2, opposite to the travelling wave 814 propagation direction D_1. The teeth 813 facilitate the motion transmission from the stator 810 to the rotor 811 by enhancing the friction between the rotor 811 and the stator 810.

The frequency and amplitude of the applied electrical voltages may be controlled and adjusted to tune the performance of the TWUSM MO, including speed, direction and accuracy of motion.

In contrast to TWUSMs, a standing wave ultrasonic motor (SWUSM) requires only a single alternating electrical voltage to operate. In response to this applied voltage, the piezoelectric actuators 801a, 801b of the stator 810 make the stator 810 vibrate according to a standing wave pattern. A standing wave is characterized by antinodes and nodes that do not travel in space. As a result, a standing wave does not have a propagation direction. The stator 810 vibrates in a way that antinodes and nodes oscillate transversely with respect to the top and bottom surface of the stator. However, antinodes and nodes do not travel circumferentially along the stator.

FIG. 57G shows an embodiment of a SWUSM. The SWUSM MO comprises a ring-shaped stator 810 with a top and a bottom surface. The stator 810 is configured to engage with a ring-shaped rotor 811. The stator 810 comprises a first set of piezoelectric actuators 801a and a second set of piezoelectric actuators 801b. An alternating electrical voltage may be selectively and exclusively applied to either set of piezoelectric actuators 801a, 801b while the other set is left floating. In such a case, the set to which the voltage is applied is referred to as active, while the other set is referred to as free. The stator 810 comprises a number of protrusions 815. The stator 810 is configured to engage with the rotor 811 via the protrusions 815. The rotor 811 is configured to be attached to the load or mechanism to be moved.

A standing wave vibration pattern may be excited in the stator 810 in response to the applied voltage. As a result, the protrusions 815 oscillate at a first angle with respect to the top surface of the stator 810 when the piezoelectric actuators 801a are active. The protrusions 815 oscillate at a second angle with respect to the top surface of the stator 810 when the piezoelectric actuators 801b are active, with the second angle different from the first angle. The first angle is such that the stator 810 imparts a clockwise rotary motion to the rotor 811. The second angle is such that the stator 810 imparts a counter-clockwise rotary motion to the rotor 811.

In the embodiment shown in FIG. 57G, the rotary ultrasonic motor MO has a rotational speed in the range 10 mrad/s-10000 mrad/s. and produces a torque in the range 20 Nmm-450 Nmm.

Rotary ultrasonic motors, such as the SWUSM or TWUSM configured to generate rotary motion could be used as the rotational electrical motor described with reference to FIGS. 5, 6, 7, 8, 9, 11-15, 17B′, 31D-31H-31K, 31Y-35, 51AA, 54B and 56-57I.

FIG. 57H shows an embodiment of a linear ultrasonic motor MO. The linear ultrasonic motor MO comprises a piezoelectric actuator 801, a pushing member 816, and a movable member 805 (or slider). The piezoelectric actuator 801 vibrates at its resonance frequency in response to an applied alternating voltage. The piezoelectric actuator 801 is attached to the pushing member 816. As a result of the vibrations in the piezoelectric actuator 801, the pushing member 816 alternatively contacts the movable member 805 and makes it move linearly by frictional coupling.

In the embodiment shown in FIG. 57H, the linear ultrasonic piezo motor MO is configured to generate linear motion with a speed in the range 4 mm/s to 100 mm/s and a force in the range 0,5 N-30 N. As the maximum stroke is limited by the length of the movable member 805 (also called a slider), there is no set limit for the maximum stroke.

This configuration of a linear ultrasonic piezo motor MO could for example be used to generate the linear motion needed for the operation of the flexible shafts (135) in the embodiments described with reference to FIGS. 11, 12A-12E, 13A-14D′, 28A—28C, 30A-31E, 31I, 31K′, 31M—31T, 32, 33 thus replacing the combination of rotational electrical motors (MO) and transmission elements transferring the rotating force to a linear force.

This configuration of a linear ultrasonic piezo motor MO could for example be used to generate the linear motion needed for the direct operation of the members in 15-17B, 17C—21, 29A—29C, 31L-31L′, 31U-31U″, thus replacing the hydraulic cylinder creating the linear motion.

The hydraulic operation devices in any of the hydraulic embodiments disclosed herein (in particular the embodiments of FIGS. 15, 17a, 17b, 18a-21, 24—26, 29a-29c and 31L′), could be operated by a piezoelectric pump.

FIG. 581 illustrates an embodiment of a piezoelectric pump configured to be implanted in the body of a patient for powering a hydraulically actuated member for stretching a stomach or intestinal wall of the patient. The piezoelectric pump P comprises a chamber 831a, a diaphragm 832a and a wall.

The wall comprises a wall portion 833a. The diaphragm 832a is connected to the wall portion 833a, such that the wall portion 833a and the diaphragm 832a enclose the chamber 831a. The wall portion 833a comprises an inlet 834a and an outlet 835a. The inlet 834a and the outlet 835a are configured to connect the chamber 831a with an inlet reservoir (not shown) and an outlet reservoir (not shown), respectively.

The diaphragm 832a is configured to bend such that the volume of the chamber varies

The piezoelectric pump is configured to be operated in a supply mode and a pump mode, as shown in FIG. 58J and FIG. 58K, respectively. In the supply mode, the diaphragm 832a bends downwards such that the volume of the chamber 831a increases, thereby decreasing the pressure in the chamber 831a compared to the inlet reservoir. Thus, an amount of fluid is supplied by the inlet reservoir to the chamber 831a via the inlet 834a. In the pump mode, the diaphragm 832a bends upwards such that the volume of the chamber 831a decreases, thereby increasing the pressure in the chamber 831a compared to the outlet reservoir. Thus, an amount of fluid is pumped from the chamber 831a to the outlet reservoir via the outlet 835a.

The piezoelectric pump comprises a driving element 836. The driving element 836 is coupled to the diaphragm 832a. A controller (cf. FIG. 58U) is configured to control the action of the driving element 836.

In some embodiments, the driving element 836 is a piezoelectric actuator, e.g. a bimorph piezoelectric actuator or any of the piezoelectric actuators herein disclosed. The driving element 836 is configured to be connected to a voltage generator. In response to an applied voltage, the driving element 836 deforms elastically, thereby imparting stress to the diaphragm 832a. As a result, the diaphragm 832a bends downwards or upwards depending on the applied voltage.

In other embodiments, the driving element 836 is driven by a piezoelectric motor. In these embodiments, the diaphragm 832a bends downwards or upwards in response to a mechanical displacement of the driving element 836 induced by the piezoelectric motor.

In any of the embodiments herein disclosed, the diaphragm 832a may comprise a bellows 852, as shown in FIG. 58L. The bellows 852 enable the contraction and expansion of the diaphragm 832a by means of the elasticity of the bellows 852. In particular, in the embodiment shown in FIG. 58L, the bellows 852 is a metal bellows 852, in particular a titanium bellows 852. As the chamber 831a comprises the titanium bellows 852, at least a portion of the wall portion 833a being in contact with the fluid in the chamber 831a comprises metal, namely titanium. Metals are generally dense, which is advantageous as fluids do not diffuse through metals as easily. This reduces the risk that fluid diffuses from the chamber 831a or that fluids diffuse into the chamber 831a. In the embodiment shown in FIG. 58L, the entire wall enclosing the chamber 831a is made from metal, in particular titanium. In embodiments in which the wall enclosing the chamber is made from a composite of metallic or non-metallic materials, the non-metallic materials could be provided as a layer or a coating applied or sprayed onto the metal. In some embodiments, at least 50% of the area of the wall enclosing the chamber 831a comprises metal and in alternative embodiments at least 80% of the area of the wall enclosing the chamber 831a comprises metal, and in yet alternative embodiments, at least 90% of the area of the wall enclosing the chamber 831a comprises metal.

Referring again to FIGS. 581-58K, the wall may further comprise a wall portion 833b and the piezoelectric pump P a chamber 831b. In this case, the diaphragm 832a is connected to the wall portion 833b such that the wall portion 833b and the diaphragm 832a enclose the chamber 831b. The chamber 831a and chamber 831b are separated by the diaphragm 832a. The chamber 831a and chamber 831b are configured to contain a first fluid and a second fluid, respectively, the two fluids being possibly different.

The chamber 831a is sealed from the chamber 831b, thereby preventing mixing of fluids between the two chambers. The fluid in the chamber 831b may be a gas, e.g. air. The sealing of the chamber 831a from chamber 831b is advantageous. In fact, a component unsuitable to be in contact with the fluid in the chamber 831a may be hosted by the chamber 831b. Such a component may be the driving element 836.

In some embodiments, the piezoelectric pump P may further comprise a diaphragm 832b connected to the wall portion 833b. In these embodiments, the wall portion 833b and the diaphragm 832b enclose the chamber 831b. Both the diaphragm 832a and diaphragm 832b are coupled to the driving element 836. In response to the action of the driving element 836, the diaphragm 832a and diaphragm 832b bend towards the same direction, i.e. upwards or downwards. The driving element 836 may be interposed between the diaphragm 832a and diaphragm 832b to prevent contact of the driving element 836 with the fluid in any of the chambers 831a and 831b. Alternatively, the wall portion 833b may be open, such that no chamber 831b is formed. Then the driving element 836 is interposed between the diaphragm 832a and diaphragm 832b to prevent contact of the driving element 836 with, for instance, the fluid in the chamber 831a or the body of the patient. In some embodiments, the diaphragm 832b may comprise bellows 852. Then, the same considerations made on the diaphragm 832a, wall portion 833a and chamber 831a in connection with FIG. 58L apply, mutatis mutandis, to the diaphragm 832b, wall portion 833b and chamber 831b.

In any of the embodiments herein disclosed in which the wall portion 833b encloses the chamber 831b, the chamber 831b may be configured to be connected to a pressure adapter 861, as shown in FIG. 58M. In particular, in the embodiment in FIG. 58M the pressure adapter 861 comprises a substantially stiff portion 862, an elastic portion 863 and a conduit 864. The elastic portion 863 is attached to the rigid portion 862, and connected to the conduit 864. The conduit 864 is configured to be connected to the chamber 831b via an opening 865 to enable variation of pressure in the chamber 831b.

The volume enclosed by the elastic portion 863 increases when the pressure in the chamber 831b increases. Vice versa, the volume enclosed by the elastic portion 863 decreases when the pressure in the chamber 831b decreases. In some embodiments, the elastic portion 863 is configured to maintain the same surface area when the volume enclosed by the elastic portion 863 varies. This is advantageous in that a fibrotic tissue, as disclosed herein, which at least partially covers the elastic portion 863 may easily adapt to the elastic portion 863.

The inlet 834a and the outlet 835a comprise an inlet valve 837a′ and an outlet valve 838a′, respectively. The inlet valve 837a′ and the outlet valve 838a′ are check valves, e.g. ball valves, bridge-type valves and cantilever-type valves. Check valves are configured to enable fluid flow in one direction while preventing backflow in the opposite direction. The inlet valve 837a′ is configured to enable fluid flow from the inlet reservoir to the chamber 831a, while preventing backflow in the opposite direction. Likewise, the outlet valve 838a′ is configured to enable fluid flow from the chamber 831a to the outlet reservoir, while preventing backflow in the opposite direction.

In other embodiments, the inlet valve 837a′ and the outlet valve 838a′ are active valves, i.e. valves controlled by a driving element. The driving element may be an actuator, e.g. a piezoelectric actuator, or a motor, e.g. a piezoelectric motor. A controller (cf. FIG. 58U) is configured to synchronize the opening and closing of the active valves so as to enable fluid transfer from the inlet 834a to the outlet 835a.

FIG. 58N and FIG. 580 illustrate an embodiment of a ball valve 841 when enabling or inhibiting fluid flow, respectively. The ball valve comprises a flow-control element 842, e.g. a ball, and a conduit 843. The conduit 843 comprises a portion 843a and a portion 843c. The conduit 843 further comprises a portion 84833b connecting the conduit portion 843a and conduit portion 843c. The flow-control element 842 is configured to move along the conduit portion 843c. The size of the flow-control element 842 is such that the flow-control element 842 cannot pass from the conduit portion 843c to the conduit portion 843a. For instance, if the flow-control element 842 is a ball, the diameter of the ball is larger than a cross-section of a conduit portion 843b. When the pressure in the conduit portion 843a is larger than the pressure in the conduit portion 843c, the flow-control element is pushed away from the conduit portion 843b and fluid flow is enabled from the conduit portion 843a to the conduit portion 843c. On the contrary, when the pressure in the conduit portion 843c is larger than the pressure in the conduit portion 843a, the flow-control element 842 seals the conduit portion 843b such that fluid flow is inhibited from the conduit portion 843c to the conduit portion 843a.

FIG. 58P and FIG. 58Q illustrate an embodiment of a piezoelectric pump operating in the supply mode and pump mode, respectively. The piezoelectric pump is configured to be implanted in the body of a patient. In this embodiment, the inlet valve 837a′ and outlet valve 838a′ (cf. FIGS. 581 to 58K) are replaced by a static element 837a″ and a static element 838a″, respectively. The static element 837a″ and static element 838a″ are configured to control the fluid flow throughout the chamber 831a while maintaining a static geometry. The static element 837a″ comprises a passage 839a″ with an increasing cross-section area when entering the chamber 831a. On the contrary, the static element 838a″ comprises a passage 840a″ with a decreasing cross-section area when entering the chamber 831a. The static element 837a″ and static element 838a″ are advantageous in that they are more resistant to wear and fatigue failure as compared to check valves and active valves. In fact, maintenance or replacement of an implantable device, such as the piezoelectric pumps disclosed herein or any of their components, pose risks for the patient.

In the supply mode (cf. FIG. 58P), the static element 837a″ acts as a diffuser and the static element 838a″ acts as a nozzle. An amount of fluid may enter the chamber 831a from the outlet 835a. However, the passage 839a″ opposes a lower flow restriction than the passage 840a″. As a result, the amount of fluid flowing into the chamber 831a via the inlet 834a is larger than the amount of fluid flowing into the chamber 831a via the outlet 835a. In the pump mode (cf. FIG. 58Q), the static element 837a″ acts as a nozzle and the static element 838a″ acts as a diffuser. An amount of fluid may exit the chamber 831a from the inlet 834a. However, the passage 840a″ opposes a lower flow restriction than the passage 839a″. As a result, the amount of fluid flowing out of the chamber 831a via the outlet 835a is larger than the amount of fluid flowing out of the chamber 831a via the inlet 834a.

An embodiment of a piezoelectric pump is illustrated in FIG. 58R. The piezoelectric pump is configured to be implanted in the body of a patient. The piezoelectric pump is configured to be operated in a double mode, as illustrated in the following. The piezoelectric pump comprises an upper portion Pa and a lower portion Pc. The upper portion Pa comprises a chamber 831a, a diaphragm 832a and a wall portion 833a. The diaphragm 832a is connected to the wall portion 833a such that the wall portion 833a and the diaphragm 832a enclose the chamber 831a. The wall portion 833a comprises an inlet 834a and an outlet 835a. The inlet 834a and the outlet 835a are configured to connect the chamber 831a with a first inlet reservoir (not shown) and first outlet reservoir (not shown), respectively. The inlet 834a comprises an inlet valve 837a′ or a static element 837a″. The outlet 835a comprises an outlet valve 838a′ or a static element 838a″.

The lower portion Pc comprises a chamber 831c, a diaphragm 832c and a wall portion 833c. The diaphragm 832c is connected to the wall portion 833c such that the wall portion 833c and the diaphragm 832c enclose the chamber 831c. The wall portion 833c comprises an inlet 834c and an outlet 835c. The inlet 834c and the outlet 835c are configured to connect the chamber 831c with a second inlet reservoir (not shown) and a second outlet reservoir (not shown), respectively. The inlet 834c comprises an inlet valve 837c′ or a static element 837c″. The outlet 835c comprises an outlet valve 838c′ or a static element 838c″. The chamber 831a and chamber 831c are configured to contain a first fluid and a second fluid, respectively, the two fluids being possibly different. The chamber 831a is sealed from the chamber 831c, thereby preventing mixing of fluids between the two chambers.

The diaphragm 832a and diaphragm 832c are coupled and configured to bend towards the same direction, i.e. upwards or downwards. A driving element 836, e.g. a piezoelectric actuator or any other driving element herein disclosed, is coupled to the diaphragm 832a and diaphragm 832c. In response to the action of the driving element 836, the diaphragm 832a and diaphragm 832c bend towards the same direction, i.e. upwards or downwards. The driving element 836 may be interposed between the diaphragm 832a and diaphragm 832b to prevent contact of the driving element 836 with the fluid in any of the chambers 831a and 831c. In some embodiments, the diaphragm 832c may comprise bellows 852.

Then, the same considerations made on the diaphragm 832a, wall portion 833a and chamber 831a in connection with FIG. 58L apply, mutatis mutandis, to the diaphragm 832c, wall portion 833c and chamber 831c.

The variation of the volume of the chamber 831a, due to a bending of the diaphragm 832a, is mirrored by an equal opposite variation of the volume of the chamber 831c, due to a bending of the diaphragm 832c. As a result, when the upper portion Pa operates in a supply mode, the lower portion Pc operates in the complementary mode, i.e. the pump mode. Vice versa, when the upper portion Pa operates in a pump mode, the lower portion Pc operates in the complementary mode, i.e. the supply mode. The double mode configuration illustrated in FIG. 58R is advantageous in that a single driving element 836 simultaneously drives two chambers. Therefore, a piezoelectric pump configured to be operated in a double mode may be cheaper, more space-efficient and more energy-efficient as compared to two independent piezoelectric pumps, each having its own driving element. It may also eliminate the need for an enclosed gas in the implant.

FIG. 58S shows an embodiment of a piezoelectric pump comprising at least a first portion PL and a last portion PR connected in series. The series may comprise additional portions. Each of the portions may be any of the embodiments disclosed with reference to FIGS. 1A to 1C and FIGS. 3A and 3B. The piezoelectric pump P is configured to be implanted in the body of a patient. The outlet of the first portion PL is configured to be connected to the inlet of the next portion of the series. The inlet of the last portion PR is configured to be connected to the outlet of the previous portion of the series. The inlet of each of the portions other than the first portion PL and last portion PR, if any, is configured to be connected to the outlet of the previous portion of the series. The outlet of each of the portions other than the first portion PL and last portion PR, if any, is configured to be connected to the inlet of the next portion of the series. The piezoelectric pump P is configured to transfer an amount of fluid from an inlet reservoir, configured to be connected to the inlet 834a of the first portion PL, to an outlet reservoir, configured to be connected to the outlet 835a of the last portion PR. This amount of fluid passes, sequentially, through the chamber 831a of the first portion PL, the chambers 831a of the next portions, if any, and the chamber 831a of the last portion PR. A controller (cf. FIG. 58U) is configured to synchronize the action of the driving elements of the portions of the series. Thus, the bending of the diaphragms of the portions of the series is synchronized so as to create the pressure required for the fluid transfer. If any of the inlet and outlets comprises an active valve, the opening and closing of any of the active valves is controlled by the controller so as to enable the fluid transfer. The series connection allows for larger pressure compared to piezoelectric pumps in which fluid is transferred from an inlet to an outlet reservoir via a single chamber.

In another embodiment (not shown), a piezoelectric pump P is provided comprising at least a first portion PL and a last portion PR connected in series. The series may comprise additional portions. The piezoelectric pump P is configured to be operated in a double mode. Each of the portions of the series may correspond to the embodiment disclosed with reference to FIG. 58R. Thus, each of the portions of the series may be configured to be operated in a double mode. Then, the outlet 835a of the first portion PL is configured to be connected to the inlet 834a of the next portion of the series, and the outlet 835c of the first portion PL is configured to be connected to the inlet 834c of the next portion of the series. The inlet 834a of the last portion PR is configured to be connected to the outlet 835a of the previous portion of the series, and the inlet 834c of the last portion PR is configured to be connected to the outlet 835c of the previous portion of the series. The inlet 834a of each of the portions other than the first portion PL and last portion PR, if any, is configured to be connected to the outlet 835a of the previous portion of the series. The inlet 834c of each of the portions other than the first portion PL and last portion PR, if any, is configured to be connected to the outlet 835c of the previous portion of the series. The outlet 835a of each of the portions other than the first portion PL and last portion PR, if any, is configured to be connected to the inlet 834a of the next portion of the series. The outlet 835c of each of the portions other than the first portion PL and last portion PR, if any, is configured to be connected to the inlet 834c of the next portion of the series. The piezoelectric pump P is configured to transfer an amount of a first fluid from a first inlet reservoir, configured to be connected to the inlet 834a of the first portion PL, to a first outlet reservoir, configured to be connected to the outlet 835a of the last portion PR. This amount of fluid passes, sequentially, through the chamber 831a of the first portion PL, the chambers 831a of the next portions, if any, and the chamber 831a of the last portion PR. The piezoelectric pump P is further configured to transfer an amount of a second fluid from a second inlet reservoir, configured to be connected to the inlet 834c of the first portion PL, to a second outlet reservoir, configured to be connected to the outlet 835c of the last portion PR. This amount of fluid passes, sequentially, through the chamber 831c of the first portion PL, the chambers 831c of the next portions, if any, and the chamber 831c of the last portion PR. A controller (cf. FIG. 58U) is configured to synchronize the action of the driving elements of the portions of the series. Thus, the bending of the diaphragms of the portions of the series is synchronized so as to create the pressure required for the fluid transfer. If any of the inlet and outlets comprises an active valve, the opening and closing of any of the active valves is controlled by the controller so as to enable the fluid transfer. The series connection allows for larger pressure as compared to piezoelectric pumps configured to be operated in a double mode in which a first fluid is transferred from a first inlet reservoir to a first outlet reservoir via a single first chamber and a second fluid is transferred from a second inlet reservoir to a second outlet reservoir via a single second chamber.

FIG. 58T shows an embodiment of a piezoelectric pump P comprising at least a first portion PU and a last portion PD connected in parallel. The parallel connection may comprise additional portions. Each of the portions may be any of the embodiments disclosed with reference to FIGS. 581 to 58K and FIGS. 58P and 58Q. The piezoelectric pump P is configured to be implanted in the body of a patient. The inlet of each portion is configured to be connected to an inlet reservoir. The outlet of each portion is configured to be connected to an outlet reservoir. The piezoelectric pump P is configured to transfer an amount of fluid from the inlet reservoir to the outlet reservoir. A controller (cf. FIG. 58U) is configured to synchronize the action of the driving elements of the portions of the parallel connection in order to ensure synergetic operation, wherein, at a given operation stage, each portion operates in the same mode, i.e. either in the supply mode or pump mode. If any of the inlet and outlets comprises an active valve, the opening and closing of any of the active valves is controlled accordingly by the controller. The parallel connection allows for a higher flow rate as compared to piezoelectric pumps in which the inlet reservoir is configured to be connected to a single inlet and the outlet reservoir is configured to be connected to a single outlet.

In another embodiment (not shown), a piezoelectric pump is provided comprising at least a first portion and a last portion connected in parallel. The parallel connection may comprise additional portions. Each of the portions may correspond to the embodiment disclosed with reference to FIG. 58R. Thus, each of the portions of the parallel connection is configured to be operated in a double mode. Then the inlet 834a and inlet 834c of each portion are configured to be connected to a first inlet reservoir and second inlet reservoir, respectively. The outlet 835a and outlet 835c of each portion are configured to be connected to a first outlet reservoir and second outlet reservoir, respectively. The piezoelectric pump is configured to transfer an amount of a first fluid from the first inlet reservoir to the first outlet reservoir. The piezoelectric pump is further configured to transfer an amount of a second fluid from the second inlet reservoir to the second outlet reservoir. A controller (cf. FIG. 58U) is configured to synchronize the action of the driving elements of the portions of the parallel connection in order to ensure synergetic operation. Thus, at a given operation stage, the chamber 831a of each portion operates in the same mode, i.e. either in the supply mode or pump mode, and the chamber 831b of each portion operates in the complementary mode. If any of the inlet and outlets comprises an active valve, the opening and closing of any of the active valves is controlled accordingly by the controller. The parallel connection allows for a higher flow rate as compared to piezoelectric pumps configured to be operated in a double mode in which the first inlet reservoir and second inlet reservoir are configured to be connected to a single first inlet and single second inlet, respectively, and the first outlet reservoir and second outlet reservoir are configured to be connected to a single first outlet and single second outlet, respectively.

The piezoelectric pumps herein disclosed are configured to be operated with a flow rate in the range 0.01 ml/min to 35 ml/min and a pressure in the range 0.2 kPa to 36 kPa.

FIG. 58U shows an embodiment of a piezoelectric pumping system configured to be implanted in the body of a patient. The system comprises a piezoelectric pump, an inlet reservoir, an outlet reservoir, and a controller. The piezoelectric pump may be any of the piezoelectric pumps herein disclosed. The controller is configured to control the piezoelectric pump. The piezoelectric pumping system may comprise a sensor and a feedback unit. The sensor is configured to measure a parameter of the piezoelectric pump, such as flow rate and pressure. Based on the sensor measurement and a set value of the measured parameter, the feedback unit sends a conditioning signal to the controller. Thus, the controller adjusts the control of the piezoelectric pump in order for the measured parameter to achieve the set value.

The hydraulic operation device or system could in any of the hydraulic embodiments disclosed herein (In particular the embodiments of FIGS. 15, 17a, 17b, 18a-21, 24—26, 29a-29c and 31L′) further comprise pressure sensor(s) for sensing the pressure in the fluid flowing in the hydraulic system of the hydraulic operation devices. The sensor(s) could for example be sensors such as the sensors described with reference to FIGS. 59A-59E. The sensor values could be used as input to an implantable controller which then could be used for controlling an implantable pump and/or an implantable valve. It may be important to measure pressure in or exerted by the medical device, as too high pressure risks hampering the blood flow to the tissue of the stomach wall, which in the long term could lead to damage of the tissue and in the worst case lead to necrosis. The controller could use a continuous or intermittent pressure signal to compute an average pressure over a time period, such as a period of more than 20 seconds, more than 1 minute, more than 3 minutes, more than 5 minutes or more than 10 minutes, as it is the average pressure over a time period that risks creating low oxygenation in the tissue and thus risks the damaging of the tissue. It may be ok that the pressure on the tissue exceeds the diastolic bold pressure, and even the systolic blood pressure, for a shorter period but not be ok if that period exceeds 20 seconds or 1 minute or 3 minutes or 5 minutes or 10 minutes. It is conceivable that the controller measures the average pressure as the integral of pressure values over a period of time.

FIG. 59A shows an embodiment of a pressure sensor 106 which could be implemented in any of the medical devices shown herein for sensing a pressure in a hydraulic fluid in the system. Pressure is an expression of the force required to stop a fluid from expanding and is stated in terms of force per unit area. The pressure sensor 106 acts as a transducer generating a signal as a function of the pressure imposed. In FIGS. 59A and 59B, a diaphragm is used as a force collector. However, it is equally conceivable that the diaphragm is replaced by e.g. a piston, a bourdon tube, or a bellows acting as force collector.

The pressure sensor 106 comprises a sensor housing 475 which comprises integrated channels. An inlet channel 470 is configured to conduct hydraulic fluid such that the hydraulic fluid is placed in contact with a diaphragm 471. The diaphragm 471 is resilient and could for example be made from a medical grade silicone material which is elastic enough such that the pressure exerted on the diaphragm 471 is transferred to a gel-like substance 473 which in turn presses on a pressure sensing element. The pressure sensing element is thus separated from the hydraulic fluid in the hydraulic operation device by the diaphragm 471. In the embodiment shown in FIG. 59A, the pressure sensing element 472 is a strain gauge which creates an electrical pressure sensor signal which is transferred to a controller by means of a lead 474. The strain gauge could be a resistive, piezoresistive or piezoelectric strain gauge, or an optical strain gauge or a capacitive strain gauge.

A resistive strain gauge uses a pressure sensing element 472 where metal strain gauges are fixated. The resistance through the metal strain gauges is changed with the elongation which is used to create the electrical pressure signal. A piezoresistive strain gauge uses the piezoresistive effect of strain gauges to detect strain due to applied pressure, resistance increasing as pressure deforms the material. Common technology types are Silicon (Monocrystalline), Polysilicon Thin Film, Bonded Metal Foil, Thick Film, Silicon-on-Sapphire and Sputtered Thin Film. A capacitive strain gauge uses the diaphragm 471 to create a variable capacitor to detect strain due to applied pressure as the capacitance decreases as pressure deforms the diaphragm 471. Common technologies use metal, ceramic, and silicon diaphragms. Electromagnetic strain gauges measure the displacement of the diaphragm 471 by means of changes in inductance (reluctance), LVDT, Hall Effect, or by eddy current principle. An optical strain gauge uses the physical change of an optical fiber to detect strain due to applied pressure. A common example of this type utilizes Fiber Bragg Gratings. The strain gauges may be connected to form a Wheatstone bridge circuit to maximize the output of the sensor and to reduce sensitivity to errors.

The pressure sensor, when implemented in any of the hydraulic operation devices shown herein, is ultimately configured to measure the pressure in the medical device which exerts pressure on the stomach wall for the purpose stretching the stomach wall to create a sensation of satiety. When a pressure is exerted on a portion of the stomach wall, the blood flow of that particular portion of the stomach wall is hampered, which creates a risk that the portion suffers from ischemia, which may cause irreversible necrosis of the restricted tissue. By measuring the pressure, the hydraulic pumps or electrically controllable valves of the system can be controlled to create pressure or strain against the stomach wall optimal for stretching the stomach wall, which in many instances is a mediation between the stretching effect while making sure that the stretching does not damage the tissue of the stomach wall.

The tissue wall is oxygenized through the circulatory blood system in which the blood pressure in a normal person is about 120 mm Hg during systole and 80 mm Hg during diastole. This means that a normal person is capable if oxygenizing tissue against a pressure not exceeding 120 mm Hg. 120 mm Hg equals 163 cm H20, which means that there is no risk, in a normal person, that tissue will suffer from ischemia as long as the pressure exerted is below 100 cm H20. As a short hampering of the blood flow in the tissue could be acceptable, but a longer could be damaging, the continuous sensing and control of the pressure exerted is important.

FIG. 59B shows an alternative embodiment of the pressure sensor, in which the pressure sensor 106 comprises a diaphragm 471 being an integrated part of the reservoir 107 in which the pressure is to be measured. A pressure sensing element 472 is connected to the diaphragm 471, such that the diaphragm 471 separates the pressure sensing element 472 from the hydraulic fluid. The pressure sensing element 472 comprises a strain gauge, for example a strain gauge functioning in accordance with one of the strain gauge principles described above. The strain gauge is connected to a controller by means of a lead 474, such that the measured pressure in the reservoir could be used in the control of the system.

In alternative embodiments, the pressure sensor could be used for measuring the pressure of a gaseous fluid. In this case, the diaphragm is in connection with an enclosed lumen configured to hold a gaseous fluid, and the pressure sensing element is configured to sense the pressure of the gaseous fluid.

The enclosed lumen configured to hold a gaseous fluid may then be in connection with a part of the hydraulic system holding the hydraulic fluid, such that the pressure in the hydraulic system can be measured indirectly by measuring the pressure of the gaseous fluid in the enclosed lumen.

FIG. 59C shows an embodiment of a pressure sensor similar to the embodiment shown in FIG. 59B, with the difference that in the embodiment of FIG. 59C, the pressure sensor 106 comprises a first 472′ and second 472″ pressure sensing element. The first pressure sensing element 472′ is configured to measure a pressure the implanted hydraulic system, e.g. by measuring the pressure in an implanted fluid reservoir 107 in fluid connection, or indirect fluid connection, with the implantable hydraulic operation device (e.g. described with reference to FIG. 15). The second pressure sensing element 472″ is configured to measure the atmospheric pressure. The reservoir 107 comprises an elastic membrane 471 being integrated in the wall of the reservoir 107, and the first pressure sensing element 472′ is configured to measure the pressure in the reservoir 107 on the first, inner, side of the elastic membrane 471 and the second pressure sensing element 472″ is configured to measure the atmospheric pressure on the second, outer, side of the elastic membrane 471. The pressure sensor 106 is connected to a controller 300, and the controller 300 is configured to derive an absolute pressure by subtracting the atmospheric pressure from the pressure in the reservoir 107. The controller then controls the pressure in the reservoir 107, and thus indirectly in the hydraulic system/hydraulic operation device, on the basis of the derived absolute pressure, or in the alternative on the basis of the received first and second input signals.

In the embodiment shown in FIG. 59C, the pressure sensor is configured to derive the pressure in the reservoir 107 by measuring the pressure in the reservoir relative to the atmospheric pressure. However, in alternative embodiments it is equally conceivable that the pressure sensor is configured to derive the pressure in the reservoir or in any other part of the hydraulic system by comparing a pressure with vacuum.

FIG. 59D shows an embodiment of a pressure sensor similar to the embodiment shown in FIG. 59B, with the difference that in the embodiment of FIG. 59D, the implant comprises a second implantable pressure sensor 106″ connected to the controller 300. The second implantable pressure sensor 106″ is configured to sense the atmospheric pressure by means of a pressure sensing element 472″. The controller 300 is as such configured to receive a second input signal related to the atmospheric pressure from the second implantable pressure sensor 106″, not necessarily placed in direct connection with the reservoir 107. In the same way as in the embodiment described with reference to FIG. 59C, the controller 300 is configured to control the pressure in the reservoir 107, and thus indirectly in the hydraulic system/hydraulic operation device, on the basis of a derived absolute pressure, or in the alternative on the basis of the received first and second input signals from the first and second pressure sensors 106′, 106″.

FIG. 59E shows an embodiment of a pressure sensor similar to the embodiment shown in FIG. 59D, with the difference that in the embodiment of FIG. 59E the system comprises a second pressure sensor 106″′ located external to the body of the patient. In the embodiment shown in FIG. 59E, the external device 320 is an external controller or communicator (further described with reference to FIGS. 65A—65FA) comprising a wireless transceiver 328 configured to communicate wirelessly with an implantable transceiver of the implantable controller 300. The second pressure sensor 106″′ located external to the body of the patient comprises a pressure sensing element 472″ configured to sense the atmospheric pressure and communicate the atmospheric pressure to the implantable controller 300 by means of the wireless communication link (328, 308) between the external device 320 and the implantable controller 300. The external device 320 may communicate the atmospheric pressure each time the patient uses, controls, programs or adjusts the implantable medical device, i.e. a signal related the atmospheric pressure may be sent together with the signal for operating, controlling or programming the medical device. As such, the implantable controller 300 may control the pressure in the reservoir 107, and thus indirectly in the hydraulic system/hydraulic operation device on the basis of a derived absolute pressure, or in the alternative on the basis of the received first and second input signals from the first and second pressure sensors 106′, 106″′, such that differences in atmospheric pressure due to weather or altitude may be considered when setting the suitable pressure in the hydraulic system/hydraulic operation device. This enables the pressure to be optimized not to damage tissue by for example hampering the blood flow.

In the alternative, the atmospheric pressure may be measured by means of the pressure sensor 106′ connected to the reservoir, or by means of a pressure sensor connected to the hydraulic system/hydraulic operation device. The method of measuring the atmospheric pressure comprises releasing the pressure from the hydraulic system/hydraulic operation device before the pressure is measured. As no pressure is added to the hydraulic system/hydraulic operation device, the atmospheric pressure will be the pressure that is measures. The pressure measured when the hydraulic system/hydraulic operation device is without added pressure can be used as a reference value against which the pressure in the hydraulic system/hydraulic operation device can be measured. This enables both the atmospheric (reference) pressure and the pressure in the hydraulic system/hydraulic operation device to be measured using the same pressure sensor, which creates a compact and efficient design. The measured reference could also be compared with the atmospheric pressure measured by a second, external pressure sensor 106″′. This comparison/calibration can be used to establish that there is no pressure in the hydraulic system/hydraulic operation device when the controller has released the pressure. The pressure applied to the hydraulic system/hydraulic operation device can be controlled either by controlling the actual pressure, or by controlling the volume of fluid pumped and/or by controlling the cross-sectional distance of the constricted urethra. I.e. if the pressure is continuously calibrated it can be established that a certain fluid level or distance leads to a specific pressure, which could make control of the device easier then control using constant pressure measurement. The controller (a computing unit of the controller) could in one embodiment create an absolute pressure by subtracting the pressure in the hydraulic system/hydraulic operation device, when substantially no pressure is exerted, from the pressure in the hydraulic system/hydraulic operation device when the pressure in the hydraulic system/hydraulic operation device has been increased. The operation device could then control the pressure in the hydraulic system/hydraulic operation device on the basis of the absolute pressure. In embodiments in which the fluid level or cross-sectional distance of the urethra is used as control value, the pressure may be used as a back-up or safety system, e.g. the pressure sensor can be set to give an alarm signal or take a specific action if the pressure increases over a set value (threshold).

In all of the described sensor embodiments above, any of the pressure sensors 106 may be a strain gauge-based pressure sensor, such as a piezoresistive or piezoelectric pressure sensor, or an optical pressure sensor, a capacitive pressure sensor, or an electromagnetic pressure sensor.

As described with further reference to FIGS. 65A—65FH, the controller 300 referenced in any of FIGS. 65A—65FH could be configured to control an electrically operable pump and/or valve to control the pressure in the hydraulic system/hydraulic operation device.

In the following a detailed description of a method and apparatus for electrically stimulating the tissue of stomach wall and thereby improve the conditions for long term implantation of the medical device will be given. The electrical electrode arrangement described and the electrical electrodes comprised in the arrangement may be implemented in any of the embodiments of the medical device described herein for the purpose of exercising the tissue wall which is in contact with the medical device.

The body tends to react to a medical implant, partly because the implant is a foreign object, and partly because the implant interacts mechanically with tissue of the body. Exposing tissue to long-term engagement with, or pressure from, an implant may deprive the cells of oxygen and nutrients, which may lead to deterioration of the tissue, atrophy and eventually necrosis. The interaction between the implant and the tissue may also result in fibrosis, in which the implant becomes at least partially encapsulated in fibrous tissue. It is therefore desirable to stimulate or exercise the cells to stimulate blood flow and increase tolerance of the tissue for pressure from the implant.

Muscle tissue is generally formed of muscle cells that are joined together in tissue that can be either striated or smooth, depending on the presence or absence, respectively, of organized, regularly repeated arrangements of myofibrillar contractile proteins called myofilaments. Striated muscle tissue is further classified as either skeletal or cardiac muscle tissue. Skeletal muscle tissue is typically subject to conscious control and anchored by tendons to bone. Cardiac muscle tissue is typically found in the heart and not subject to voluntary control. A third type of muscle tissue is the so-called smooth muscle tissue, which is typically neither striated in structure nor under voluntary control. Smooth muscle tissue can be found within the walls of organs and in for example the stomach wall.

The contraction of the muscle tissue may be activated both through the interaction of the nervous system as well as by hormones. The different muscle tissue types may vary in their response to neurotransmitters and endocrine substances depending on muscle type and the exact location of the muscle.

A nerve is an enclosed bundle of nerve fibers called axons, which are extensions of individual nerve cells or neurons. The axons are electrically excitable, due to maintenance of voltage gradients across their membranes, and provide a common pathway for the electrochemical nerve impulses called action potentials. An action potential is an all-or-nothing electrochemical pulse generated by the axon if the voltage across the membrane changes by a large enough amount over a short interval. The action potentials travel from one neuron to another by crossing a synapse, where the message is converted from electrical to chemical and then back to electrical.

The distal terminations of an axon are called axon terminals and comprise synaptic vesicles storing neurotransmitters. The axonal terminals are specialized to release the neurotransmitters into an interface or junction between the axon and the muscle cell. The released neurotransmitter binds to a receptor on the cell membrane of the muscle cell for a short period of time before it is dissociated and hydrolyzed by an enzyme located in the synapse. This enzyme quickly reduces the stimulus to the muscle, which allows the degree and timing of muscular contraction to be regulated delicately.

The action potential in a normal skeletal muscle cell is similar to the action potential in neurons and is typically about −90 mV. Upon activation, the intrinsic sodium/potassium channel of the cell membrane is opened, causing sodium to rush in and potassium to trickle out. As a result, the cell membrane reverses polarity and its voltage quickly jumps from the resting membrane potential of −90 mV to as high as +75 mV as sodium enters. The muscle action potential lasts roughly 2-4 ms, the absolute refractory period is roughly 1-3 ms, and the conduction speed along the muscle is roughly 5 m/s.

This change in polarity causes in turn the muscle cell to contract.

The contractile activity of smooth muscle cells is typically influenced by multiple inputs such as spontaneous electrical activity, neural and hormonal inputs, local changes in chemical composition, and stretch. This in contrast to the contractile activity of skeletal and cardiac muscle cells, which may rely on a single neural input. Some types of smooth muscle cells are able to generate their own action potentials spontaneously, which usually occur following a pacemaker potential or a slow wave potential. However, the rate and strength of the contractions can be modulated by external input from the autonomic nervous system. Autonomic neurons may comprise a series of axon-like swellings, called varicosities, forming motor units through the smooth muscle tissue. The varicosities comprise vesicles with neurotransmitters for transmitting the signal to the muscle cell.

The muscle cells described above, i.e., the cardiac, skeletal and smooth muscle cells are known to react to external stimuli, such as electrical stimuli applied by electrodes. A distinction can be made between stimulation transmitted by a nerve and direct electrical stimulation of the muscle tissue. In case of stimulation via a nerve, an electrical signal may be provided to the nerve at a location distant from the actual muscle tissue, or at the muscle tissue, depending on the accessibility and extension of the nerve in the body. In case of direct stimulation of the muscle tissue, the electrical signal may be provided to the muscle cells by an electrode arranged in direct or close contact with the cells. However, other tissue such as fibrous tissue and nerves may of course be present at the interface between the electrode and the muscle tissue, which may result in the other tissue being subject to the electrical stimulation as well.

In the context of the present application, the electrical stimulation discussed in connection with the various aspects and embodiments may be provided to the tissue in direct or indirect contact with the medical device. Preferably, the electrical stimulation is provided by one or several electrode elements arranged at the interface or contact surface between the medical device and the tissue. Thus, the electrical stimulation may, in terms of the present disclosure, be considered as a direct stimulation of the tissue. Particularly when contrasted to stimulation transmitted over a distance by a nerve, which may be referred to as an indirect stimulation or nerve stimulation.

Hence, an electrode arrangement comprising one or several electrode elements may be arranged in, partly in, on, or in close vicinity of the tissue that is to be exercised by means of an electrical signal. Preferably, the electrode may be arranged to transmit the electrical signal to the portions of the tissue that is affected, or risks to be affected, by mechanical forces exerted by the medical implant. Thus, the electrode element may be considered to be arranged between the implanted device and the tissue against which the device is arranged to rest when implanted.

During operation of the medical device, or the electrode arrangement, the electric signal may cause the muscle cells to contract and relax repeatedly. This action of the cells may be referred to as exercise and may have a positive impact in terms of preventing deterioration and damage of the tissue. Further, the exercise may help increasing tolerance of the tissue for pressure and mechanical forces generated by the medical device.

The interaction between the implanted electrode element and the tissue of the stomach wall is to a large extent determined by the properties at the junction between the tissue and the electrode element. The active electrically conducting surface of the electrode element (in the following referred to as “metal”, even though other materials is equally conceivable) can either be uncoated resulting in a metal-tissue interface, or insulated with some type of dielectric material. The uncoated metal surface of the electrode element may also be referred to as a bare electrode. The interface between the electrode element and the tissue may influence the behavior of the electrode element, since the electrical interaction with the tissue is transmitted via this interface. In the biological medium surrounding the electrode element, such as the actual tissue and any electrolyte that may be present in the junction, the current is carried by charged ions, while in the material of the electrode element the current is carried by electrons. Thus, in order for a continuous current to flow, there needs to be some type of mechanism to transfer charge between these two carriers.

In some examples, the electrode element may be a bare electrode wherein the metal may be exposed to the surrounding biological medium when implanted in, or at the muscle tissue that is to be stimulated. In this case there may be a charge transfer at a metal-electrolyte interface between the electrode element and the tissue. Due to the natural strive for thermodynamic equilibrium between the metal and the electrolyte, a voltage may be established across the interface which in turn may cause an attraction and ordering of ions from the electrolyte. This layer of charged ions at the metal surface may be referred to as a “double layer” and may physically account for some of the electrode capacitance.

Hence, both capacitive faradaic processes may take place at the electrode element. In a faradaic process, a transfer of charged particles across the metal-electrolyte interface may be considered as the predominant current transfer mechanism. Thus, in a faradaic process, after applying a constant current, the electrode charge, voltage and composition tend to go to constant values. Instead, in a capacitive (non-faradaic) process charge is progressively stored at the metal surface and the current transfer is generally limited to the amount which can be passed by charging the interface.

In some examples, the electrode element may comprise a bare electrode portion, i.e., an electrode having an uncoated surface portion facing the tissue such that a conductor-tissue interface is provided between the electrode element and the tissue when the electrode element is implanted. This allows for the electric signal to be transmitted to the tissue by means of a predominantly faradaic charge transfer process. A bare electrode may be advantageous from a power consumption perspective, since a faradaic process tend to be more efficient than a capacitive charge transfer process. Hence, a bare electrode may be used to increase the current transferred to the tissue for a given power consumption.

In some examples, the electrode element may comprise a portion that is at least partly covered by a dielectric material so as to form a dielectric-tissue interface with the muscle tissue when the electrode is implanted. This type of electrode element allows for a predominantly capacitive, or non-faradaic, transfer of the electric signal to the muscle tissue. This may be advantageous over the predominantly faradaic process associated with bare electrodes, since faradaic charge transfer may be associated with several problems. Example of problems associated with faradaic charge transfer include undesirable chemical reactions such as metal oxidation, electrolysis of water, oxidation of saline, and oxidation of organics. Electrolysis of water may be damaging since it produces gases. Oxidation of saline can produce many different compounds, some of which are toxic. Oxidation of the metal may release metal ions and salts into the tissue which may be dangerous. Finally, oxidation of organics in a situation with an electrode element directly stimulating tissue may generate chemical products that are toxic.

These problems may be alleviated if the charge transfer by faradaic mechanisms is reduced, which may be achieved by using an electrode at least partly covered by a dielectric material. Preferably, the dielectric material is chosen to have as high capacitance as possible, restricting the currents flowing through the interface to a predominantly capacitive nature.

Several types of electrode elements can be combined with the present disclosure. The electrode element can for example be a plate electrode, comprising a plate-shaped active part forming the interface with the tissue. In other examples, the electrode may be a wire electrode, formed of a conducting wire that can be brought in electrical contact with the tissue. Further examples may include needle- or pin-shaped electrodes, having a point at the end which can be attached to or inserted in the muscle tissue. The electrodes may for example be encased in epoxy for electrical isolation and protection and comprise gold wires or contact pads for contacting the muscle tissue. Some of these examples of electrodes, methods of stimulating using electrodes, and how the electrode arrangements can be arranged in connection with implantable medical devices will be discussed below with reference to FIGS. 60—72.

FIG. 60 shows the embodiment of the implantable medical device further described with reference to FIGS. 4a-4c. The medical device 10 of FIG. 60 further comprises an electrode arrangement comprising two electrodes E1, E2 for electrically stimulating the tissue of the stomach wall SW for exercising the muscle tissue to improve the conditions for long term implantation of the medical device, or for increasing the effect of the stretching of the stomach wall. In the embodiment of FIG. 60, the electrode arrangement is arranged on the members 101a, 101b and thus placed in abutment and in electrical connection with the tissue of the stomach wall SW. Each of the two electrode elements E1, E2 are connected to a stimulation controller 350 by means of electrical leads 474. The stimulation controller 350 is configured to be operably connected to the electrode arrangement for controlling the electrical stimulation of the tissue. In the embodiment shown in FIG. 60, the stimulation controller 350 is configured to control the electrical stimulation such that the tissue is stimulated by a series of electrical pulses. In the embodiment shown in FIG. 60, the pulses comprise a pulse of a first polarity followed by a pulse of a second, reversed polarity, and the pulsed electrical stimulation signal generated comprises a pulse frequency of 0.01-150 Hz. In the embodiment shown in FIG. 60, the electrical stimulation signal comprises a pulse duration of 0.01-100 ms and a pulse amplitude of 1-15 mA. More specifically, in the embodiment of FIG. 60, the electrical stimulation signal comprises a pulse frequency of 0.15-0.25 Hz, a pulse duration of 20-30 ms and a pulse amplitude of 3-10 mA. Further, in the embodiment of FIG. 60, the electrical stimulation signal comprises a build-up period of 0.01-2 s in which the amplitude is gradually increasing, a stimulation period of 1-60 s, and a stimulation pause of 0.01-60 s, wherein the electrical signal comprises a pulse frequency of 1-50 Hz and a pulse duration of 0.1-10 ms.

The stimulation controller 350 of FIG. 60 is integrated in an implantable controller, such as the implantable controller described with reference to FIGS. 65A—65FH, and the stimulation controller may be configured to receive input from a wireless remote control, directly or via a receiver of the implantable controller, for controlling the stimulation or for programming a stimulation routine for exercising the muscle tissue to improve the conditions for long term implantation of the medical device 10. The programming of a stimulation routine could for example be the programming of the frequency of the stimulation, or the current and/or voltage of the stimulation.

In the embodiment shown in FIG. 60, the implantable medical device 10 further comprises an implantable sensor S1 configured to sense actions potentials generated by pacemaker cells of the tissue of the stomach wall SW. The implantable sensor S1 is connected to the main portion M and connected to the stimulation controller 350 by means of a sensor lead 474. The stimulation controller 350 is configured to control the electrical simulation based at least partly on the sensed action potentials and is configured to generate electrical pulses amplifying the sensed action potentials. The implantable sensor may be implemented in any of the embodiments of medical devices 10 for controlling the electrical stimulation by the electrode elements, which also may be implemented in any of the embodiments of medical devices described herein.

At least one stimulation electrode (E1) could be applied between a member and the tissue wall of the stomach, such as shown in FIG. 60, in any of the embodiments of medical devices for stretching the stomach wall described herein. More specifically, at least one stimulation electrode could be placed on a member of the embodiments shown in FIGS. 1A, 1B, 2A, 3A, 4A, 5, 6, 7, 8, 9, 11, 12A, 13A, 14A, 14B, 14C, 14D, 15, 17A, 21, 22, 23, 24, 25, 26, 27, 28A, 29A, 30A, 31, 31C, 31D, 31F, 31G, 31H, 31I,31J, 31K, 31K′, 31L, 31L′, 31M, 31N, 31O, 31P, 31Q-31T, 31U, 31V, 31X, 31Y, 33A-33H and 34A-341 such that the electrode is placed in connection with the stomach wall of the patient when the medical device is implanted. In all embodiments, the at least one electrode can be used for exercising the muscle tissue that is mechanically affected by the members, or for increasing the satiety-creating effect of the stretching by further stimulating the stretch receptors of the stomach wall.

In alternative embodiments, the at least one stimulation electrode E1 could be replaced of complemented with a vibrator configured to mechanically stimulate a wall portion of the stomach, such that the wall of the stomach is actively stretched and mechanically stimulated simultaneously for creating a sensation of satiety. The vibrator may be a vibrator such as further described with reference to FIGS. 64X—64Z and could be configured to be operated by a piezoelectric operation device, such as any of the piezoelectric motors described with reference to FIGS. 58A-58H.

The electrode E1 or vibrator may be connected to a wireless energy receiver configured to receive energy for powering the electrode or vibrator wirelessly.

The member including the electrode and/or vibrator may be configured to be at least partially invaginated by the tissue of the stomach wall using stomach-to-stomach sutures or staplers.

The vibrator may be configured to vibrate at a frequency in the range of 1-100 Hz, such as in the range of 1-20 Hz, and with a period of 0.01-1 seconds, such as of 0.05-1 seconds. The vibrator may be configured to vibrate at an amplitude such that the tissue in the stomach wall is displaced at least 1 mm.

The member 101a may further comprises a second electrode, and the first and second electrodes may be configured for successively electrically stimulating two portions of the tissue wall of the stomach. The first and second electrodes may be placed on the member 101a at a distance from each other being at least 5 mm, and/or a distance from each other corresponding to a wavelength of a wave pattern of a movement of smooth muscles of the stomach wall, or by a multiple of such a wavelength.

The stimulation controller 350 may be configured to send stimulation signals to the first and second electrode in pulses, or pulse trains. And the pulses or pulse trains may have a frequency being determined based on a frequency of the wave pattern of a movement of smooth muscles of the stomach. The stimulation controller 350 is configured to send stimulation signals to the first and second electrode in pulses or pulse trains having a pulse separation or pulse train separation of 0,5-10 s, such as 1-8 s, such as 2-6 s.

FIG. 61A is an example of a bipolar electrode arrangement, comprising a first and a second electrode element E1, E2 that can be connected to different electrical potentials. Thus, the first electrode element E1 can be operated as an anode and the second electrode element E2 can be operated as a cathode. The electrode elements E1, E2 may be attached directly to an outer surface of a member of the medical device, such as disclosed with reference to FIG. 60. In some examples the electrode elements E1, E2 may be arranged on a support, such as a flexible patch, which may be configured to be attached to the medical device. The electrode arrangement 353 can be arranged between the medical device/member (101a, 101b) and the tissue (such as disclosed with reference to FIG. 60) and may in some examples be provided as a separate, physically distinct item and in other examples be integrated in the medical device.

The electrode arrangement 353 may comprise one or several contact pads for increasing the contact surface between the electrode and the tissue when implanted. During operation, the electrical signal may be delivered to the muscle tissue by means of the first and second electrode elements E1, E2 so as to stimulate contraction of the muscle cells.

FIG. 61B is another example of an electrode arrangement 353, which in the present example may be a unipolar electrode element E1. The electrode element E1 may for example be operated as a cathode when implanted. The electrode element E1 may be formed of a flat, coiled wire for increasing the contact surface between the electrode element E1 and the tissue. Further, the coiled configuration allows for a certain mechanical flexibility of the electrode element E1 such that it can follow the muscle tissue during contraction and relaxation.

FIG. 61C illustrates the end portion of a needle- or pin-shaped electrode arrangement 353, wherein the active portion of the electrode element E1 is provided as a bare electrode surface 354 at the end of the electrode element E1, protruding from an insulation 355 covering the rest of the electrode element E1. Thus, when implanted at or in the muscle tissue, the active, bare electrode surface 354 of the electrode element E1 may form a metal-tissue interface with the muscle tissue, wherein the interface may surround the end portion of the electrode element E1 so as to provide a relatively large contact surface.

The present example is advantageous in that it can be inserted into the tissue, thereby allowing for a selective stimulation at a certain depth of the tissue.

FIG. 61D shows a similar electrode element as the one in FIG. 61C, with the difference that the present electrode element E1 comprises an active portion that is covered by a dielectric material 356 so as to protect the electrode material from deterioration and to facilitate capacitive current transfer. The dielectric material 356 may for example be electrochemically deposited tantalum oxide, which allows the electrical charge to pass through the interface but reduces the risk for electrode corrosion, gas formation and metabolite reactions.

It will be appreciated that both faradaic and capacitive mechanisms may be present at the same time, irrespectively of the type of electrode used. Thus, capacitive charge transfer may be present also for a bare electrode forming a metal-tissue interface, and faradaic charge transfer may be present also for a coated electrode forming a dielectric-tissue interface. It has been found that the faradaic portion of the current delivered to the muscle tissue can be reduced or even eliminated by reducing the duration of the pulses of the electric signal. Reducing the pulse duration has turned out to be an efficient way of increasing the portion of the signal which can be passed through the interface as a capacitive current, rather than by a faradaic current. As a result, shorter pulses may produce less electrode and tissue damage.

The capacitive portion of the current may further be increased, relative to the faradaic portion, by reducing the amplitude of the current pulses of the electrical signal. Reducing the amplitude may reduce or suppress the chemical reactions at the interface between the electrode and the tissue, thereby reducing potential damage that may be caused by compounds and ions generated by such reactions.

In one example, the electrical stimulation may be controlled in such a manner that a positive pulse of the electrical signal is followed by a negative pulse (or, put differently, a pulse of a first polarity being followed by a pulse of a second, reversed polarity), preferably of the same amplitude and/or duration. Advantageously, the subsequent negative (or reversed) pulse may be used to reverse or at least moderate chemical reactions or changes taking place in the interface in response to the first, positive pulse. By generating a reversed pulse the risk of deterioration of the electrode and/or the tissue at the interface between the electrode and the muscle tissue may be reduced.

FIG. 62 shows an example of a pulsed electrical signal to be applied to an electrode for electrically stimulating muscle tissue via an electrode-tissue interface as discussed above. The electrical signal may be generated by a stimulation controller arranged outside the body or implanted in the body (as described with reference to FIG. 60). The stimulation controller may be operatively connected to the electrode element by means of a lead, and the electrical signal shown in the present figure may either reflect the signal as generated at the stimulation controller, or the signal as delivered to the electrode element at the electrode-tissue interface. The characteristics of the electrical signal may be selected and varied determined on the electrical and properties at the electrode-tissue interface and on the actual response of the tissue. The electrical stimulation delivered to the muscle cells may depend on several factors, such as the configuration and placement of the electrode element at the tissue, the presence of fibrous material at the interface, the composition of the electrolyte in the interface, accumulation of non-conducting material on the electrode surfaces, etcetera. It is therefore suggested that the characteristics of the electric signal, as shown in the present figure, be selected and varied based on an observed or estimated response from the stimulated tissue.

In the present example, the electrical signal is a pulsed signal comprising square waves PL1, PL2, PL3, PL4. However, other shapes of the pulses may be employed as well. The pulse signal may be periodic, as shown, or may be intermittent (i.e., multiple series of pulses separated by periods of no pulses). The pulses may have an amplitude A, which may be measured in volts, ampere or the like. Each of the pulses of the signal may have a pulse width D. Likewise, if the signal is periodic, the pulse signal may have a period F that corresponds to a frequency of the signal. Further, the pulses may be either positive or negative in relation to a reference.

The pulse frequency may for example lie within the range of 0.01-150 hertz. More specifically, the pulse frequency may lie within at least one of the ranges of 0.1-1 Hz, 1-10 Hz, 10-50 Hz and 50-150 Hz. It has been observed that relatively low pulse frequencies may be employed to imitate or enhance the slow wave potential associated with pacemaker cells of the smooth muscle tissue. Thus, it may be advantageous to use relatively low pulse frequencies, such as 0.01-0.1 Hz or frequencies below 1 Hz or a few Hz for such applications.

The pulse duration may for example lie within the range of 0.01-100 milliseconds, such as 0.1-20 milliseconds (ms), and preferably such as 1-5 ms. The natural muscle action potential has in some studies been observed to last about 2-4 ms, so it may be advantageous to use a pulse duration imitating that range.

The amplitude may for example lie within the range of 1-15 milliamperes (mA), such as 0.5-5 mA in which range a particularly good muscle contraction response has been observed in some studies.

In a preferred, specific example the electrical stimulation may hence be performed using a pulsed signal having a pulse frequency of 10 Hz, a pulse duration of 3 ms and an amplitude of 3 mA.

FIG. 63 shows an example of a pulsed signal, comprising build-up period X1, in which the amplitude is gradually increasing, a stimulation period X2 during which the muscle tissue is exposed to a contracting stimulation signal, a ramp down period X3 in which the amplitude is gradually decreasing, and a stimulation pause X4 before a new build-up period is initiated. The build-up period may for example be 0.01-2 seconds, the stimulation period 1-60 seconds, the ramp-down period 0.01-2 seconds, and the stimulation pause 0.01-60 seconds. The pulse frequency may for example be 1-50 Hz, the pulse duration 0.1-10 milliseconds and the amplitude during the stimulation period be 1-15 milliampere. The stimulation of skeletal muscle tissue may for example be performed using a frequency of 50 Hz and pulses having a duration of 100 ρs. The current amplitude may be 1, 2.5, 7.5 or 10 mA. In particular, a desired muscle contraction response has been experimentally observed within a range of 0.5 to 5.0 mA.

In the present example, a coiled electrode may be used as a cathode. Another example design is a multi-stranded wire arranged in a helical design. They can be imbricated in the muscular wall of the organ, such as the urinary bladder, and can be stimulated in any desired pattern. The stimulus parameters may for example be biphasic pulses, 10 to 40 Hz, lasting 0.1 to 5 ms, with a current density of 3 to 5 mA/cm2.

FIG. 64 is a schematic outline of a system for electrically stimulating or exercising muscle cells to increase tolerance of the tissue for pressure from the medical device. The system may be used in combination with the medical device and may in some examples be comprises in such a medical device.

The system comprises an electrode arrangement 353 which may be similarly configured as the electrodes arrangements/electrode elements discussed above in connection with the previous examples, an energy storage unit 40 for providing the electrical energy required for generating the electrical signal, and a stimulation controller 350 controlling the generation of the electrical signal.

The electrode arrangement, which may comprise one or several electrode elements, such as a bare electrode or an electrode at least partly covered by a dielectric material, may be configured to be implanted in the muscle tissue to be stimulated, or to engage the muscle, so as to form an electrode-tissue interface through which the stimulating signal may be transferred. Alternatively, or additionally, the electrode element may be arranged in close vicinity to the muscle tissue such that an electrical coupling between the electrode element and the muscle tissue may be established. This may for example be the case when other tissue, such as connective tissue, is present between the implanted device and the muscle tissue.

The electrode may be electrically connected to the energy storage unit 40, for example by means of a wiring or a lead, such that the electrical signal may be transferred to the electrode-tissue interface. In some examples, the electrode may be integrated with or attached to the medical device, such that the electrode when implanted in the patient is arranged at the interface between the members and the muscle tissue. The electrode can thereby be used for exercising the muscle tissue that is mechanically affected by the members, or for increasing the satiety-creating effect of the stretching by further stimulating the stretch receptors.

The energy storage unit 40 may for example be of a non-rechargeable type, such as a primary cell, or of a rechargeable type, such as a secondary cell. The energy storage unit 40 may be rechargeable by energy transmitted from outside the body, from an external energy storage unit, or be replaced by surgery. Further, the electrode arrangement 353 may be operably connected to a stimulation controller 353, which may comprise an electrical pulse generator, for generating the electrical pulse. The stimulation controller 350 may be integrated with the energy storage unit 40 or provided as a separate, physically distinct unit which may be configured to be implanted in the body or operate from the outside of the body. In case of the latter, is may be advantageous to allow the external control unit to communicate wirelessly with the stimulation controller for example by means of a communication unit of a more general controller (for example described with reference to FIGS. 65A—65FH).

The system may according to some examples comprise a sensor S1 that is configured to sense a physical parameter of the body and/or the medical device. The sensor S1 may for example be employed to sense or detect a bodily response to the electrical stimulation, such as for example a contraction of the stimulated muscle tissue. In an example, the sensor S1 may be configured to sense action potentials that are being sent to the muscle tissue. The action potentials may for example be generated by pacemaker cells of the muscle tissue, which may be registered by the sensor S1 and transmitted to the stimulation controller 350. The stimulation controller 350 may use the received signal when controlling the energy storage unit 40, such that the generated electrical signal amplifies the sensed action potentials.

In an embodiment, there is provided a stimulation device for delivering electrical stimulation to tissue in the stomach of the patient, such as also explained with reference to FIG. 60, to generate a signal in the vagal afferents innervating the stomach tissue, to thereby create a sensation of satiety and/or reduce the appetite in a patient.

An exemplary system for generating a signal in the vagal afferents innervating the gastric musculature in the stomach or the intestine of a patient will now be discussed with reference to FIGS. 64B-64F. FIG. 64B is a schematic illustration of a particular example of the system and its interaction with the body of the patient P, and more particularly with a stomach or intestinal tissue 230 innervated by a vagal afferent 231. As indicated in the figure, the vagal afferent 231 may extend between the stomach tissue 230 and the central nervous system (CNS) 233. In the present example, the origin of the illustrated nerve 231 is represented by item 233. It will be appreciated that the stomach tissue 230 and the CNS 233 are merely schematically indicated in the present figure.

As illustrated, the system comprises a stimulation device 40 configured to deliver, directly or indirectly, a first simulation signal to the vagal afferent 231 innervating stomach tissue 230. This can be achieved by delivering stimulation directly to the vagal afferent, or to the tissue of the stomach. The system further comprises a control unit, or controller 240, configured to control an operation of the stimulation device 40 such that the stimulation signal stimulates an activity of the vagal afferent 231. A signal that results in an activation of the nerve may be referred to as an activation signal. By activation is generally understood the generation of a nerve signal, i.e., action potentials travelling in the nerve.

The nervous response may typically be determined by, inter alia, a frequency content of the signal. The signal may be a periodic signal, including at least one of: a variable frequency component, a variable duty cycle component, a variable amplitude component, and a variable pause component.

It may therefore be beneficial to measure the effect of the stimulation to determine whether the treatment has an intended effect or not and to provide feedback that can be used to adjust the characteristics of the stimulation signal. For example, the measured effect may be used as feedback in a closed loop control of the stimulation device 40. This will be discussed in further detail later in the present disclosure.

A stimulation signal may have different characteristics depending on the desired neural response to be achieved. As already mentioned, these characteristics may relate to amplitude, frequency, waveform, polarity, and duty cycle. The duty cycle may be understood as the ratio of the time that the signal is ‘on’, i.e., active, to the total time of one cycle (period). For a pulsed signal, this would correspond to the ratio of the pulse length to the length a cycle. The duty cycle is usually expressed as a percentage or a fraction. For example, a 50% duty cycle may be understood as the signal being ‘on’ for half of the cycle and ‘off’ for the other half. The duty cycle of an electric stimulation signal can influence the energy delivered to the tissue (or at least the energy to which the tissue is exposed). A higher duty cycle means that the tissue may be exposed to more electric charges and energy, which can increase the stimulation effect, whereas a lower duty cycle means that the tissue may be exposed to less electric charge and energy. A lower duty cycle may mean that the tissue has more time to recover and adapt to the stimulation, which can reduce the risk of tissue damage or fatigue. Different types of tissues may require different duty cycles for optimal stimulation. Electric stimulation signals with low duty cycles (less than 10%) have been shown to promote cell regeneration, proliferation, and growth, whereas electric signals with high duty cycles (more than 50%) have been shown to inhibit cell growth.

Accordingly, the pause component of a stimulation signal, describing the time interval between two consecutive pulses or two consecutive pulse trains, can affect the stimulation of tissue in several ways. Increasing the pause component may facilitate recovery from the previous stimulation and reduce the risk of overstimulation or fatigue. A longer pause component can reduce the risk of tissue damage or adaptation, while a shorter pause component can increase the stimulation effect. Furthermore, the pause component may influence the net charge delivered to the tissue and the electrochemical reactions at the electrode-tissue interface. A longer pause component can allow the charge to dissipate and the pH to normalize, while a shorter pause component can cause charge accumulation and pH changes. The pause component may in some examples range from 0.1-10 seconds, such as 0.5-2 seconds, depending on the tissue type.

In an example the stomach tissue 230, innervated by the vagal afferent 23, or the vagal afferent itself 231 can be activated or stimulated by an activation signal comprising a frequency in the range of 0.1-100 Hz, such as 1-50 Hz. Such a signal may be referred to as a low-frequency signal. The activation signal may comprise a voltage in the range of 1-15 V, such as about 10 V and a current in the range of 1-50 mA, such as 2-4 mA.

The control unit 240 may be configured to control the operation of the stimulation device 40.

The control unit 240 may as well be configured to control the operation of the stimulation device 40 to provide a signal for activating the vagal afferent 231 innervating the stomach tissue 230.

The stimulation device 40 may comprise circuitry and a power source for generating the stimulation signal electrical stimulation signals. The circuitry and, optionally, the power source may be arranged within a housing which may be implantable in the body of the patient. Electrical leads may be provided to connect the circuitry to a signal generating means 210 arranged at the stomach tissue. In case of an electric stimulation signal, the signal generating means 210 may comprise an electrode arrangement.

At least parts of the stimulation device 40, such as a housing and/or energy source, may be implanted in the stomach tissue may be implanted in fat tissue of the patient, be anchored to bone tissue, or implanted subcutaneously.

The control unit 240 may be integrated with the stimulation device 40, such as arranged within the same housing as the electric circuitry, or the energy source mentioned above. In other examples, the control unit 240 may be arranged separately or remotely, i.e., at a different physical location than the stimulation device 40. In the latter case, the control unit 240 may be communicatively coupled to the stimulation device 40 by means of a wired or wireless connection. The control unit 240 may hence be arranged within the patient's body or externally, i.e., outside the body of the patient P.

FIGS. 64C-64F show examples of electrode arrangements which may be implemented in any of the stimulation devices 40 discussed in the present disclosure.

FIG. 64C is an example of a bipolar electrode arrangement comprising a first and a second electrode element E1, E2, having a plurality of contact portions 122a which can be arranged to abut the vagal afferent 231, or be arranged to touch the stomach tissue 230 innervated by the vagal afferent 231.

The electrode arrangement may be operated as a bipolar electrode arrangement by connecting the first and second electrode elements E1, E2 to different electrical potentials. Thus, the first electrode element E1 can be operated as an anode and the second electrode element E2 as a cathode. The electrode elements E1, E2 may be attached directly to a support structure 126, such as a patch, or a cuff 215 as shown in FIG. 64F. The electrode arrangement may comprise one or several contact pads, or contacting portions 122a, for increasing the contact surface between the electrode and the tissue when implanted. During operation, the stimulation signal may be delivered to the tissue, i.e., the nerve or the stomach tissue, by means of the first and/or second electrode elements E1, E2 to activate action potentials in the nerve directly or to stimulate the stomach tissue 230 innervated by the vagal afferent 231. It may be contemplated that the electrode arrangement only comprises the first electrode element E1

FIG. 64E is another example of an electrode arrangement of an electrical stimulation device 40 as discussed above. In the present example, the electrode arrangement may be operated as a unipolar electrode element or as a bipolar electrode arrangement. The electrode arrangement comprises a first electrode element E1 and a second electrode element E2 which may be formed of a wire or electrical lead arranged in a flat, coiled structure for increasing the contact surface between the electrode elements E1, E2 and the nerve tissue. The electrode elements E1, E2 may be arranged on a flexible and/or stretchable support or patch 216, allowing it to conform to the shape of the tissue to which it is attached and move with any movement of the same. The coiled configuration allows for a certain mechanical flexibility of the electrode elements E1, E2 such that they can move with any movement or deformation of the support 215. It may be contemplated that the electrode arrangement only comprises the first electrode element E1

FIG. 64E illustrates the end portion of a needle- or pin-shaped electrode element E1, wherein the active portion of the electrode element Elis provided as a bare electrode surface 123 at the end of the electrode element E1. Thus, when implanted at or in the tissue, the active, bare electrode surface 123 of the electrode element E1 may form a metal-tissue interface with the tissue. The tip 123 of the electrode element E1 may be inserted into the nerve tissue 231 or arranged to abut an outer surface of the nerve 231. The present example may form a unipolar electrode, and may require another electrode, such as a ground electrode, to be arranged elsewhere on the patient's body to form a closed electric circuit. The completing electrode may, for instance, be formed by a housing of the stimulation device 40 or the vibration device 110.

FIG. 64F shows an electrode arrangement 210, 220 comprising a cuff 215 for attachment around the nerve 231, 232. The cuff 215 forms a support structure for the electrode element(s) E1, E2 and allows them to be touching the outer surface of the nerve 231. The cuff 215 may be configured to at least partly surround or enclose a circumference of the nerve 231, 232 and may comprise an opening or slit allowing the cuff 215 to be fitted around the nerve 231.

One or more of the first and second electrode arrangements 210, 220 may comprise one or more of the electrode elements E1, E2 described above. The electrode elements E1, E2, which also may be referred to as stimulation electrodes, may be spaced apart along the vagal afferent nerve 231. This allows the stimulation device 40 to generate the stimulation signal(s) such that a first one of the stimulation electrodes E1, E2 serves as a cathode and a second one of the stimulation electrodes E1, E2 serves as an anode. It will be appreciated that further electrodes (not shown) may be provided, such as a third electrode, a fourth electrode, and a fifth electrode. Each of the third, fourth and fifth electrode may serve as an anode or a cathode during operation of the stimulation device 40

Various measures may be taken to ensure electrical safety and to comply with different regulatory frameworks. Direct current (DC) flowing through electrodes or other implanted parts of a system according to any of the aspects of the present disclosure may be a safety concern, as it may cause tissue damage. For example, it has been reported that DC levels as low as 2-3 pA may cause pathological changes in nerve tissue. It is therefore desirable to limit leakage current (DC) to 1IpA or less, such a 0.1 pA or less. This may be achieved by means of a capacitor, also referred to as a DC blocking capacitor, which may be arranged in any of the current pathways. Specifically, the capacitor may be connected in series with two or more electrodes of the implant, such as the ones employed to apply a stimulation signal or a measuring signal. Furthermore, the capacitor may be connected in series with a part of the implant (such as an electrode, an energy source, or a housing) and the body of the patient, thereby reducing any current that might flow between the implant and tissue of the patient.

A further advantage of the capacitor relates to prevention of charge accumulation on the electrodes. By coupling a capacitor to the electrodes, the capacitor may help dissipating accumulated charge from the electrodes, thereby allowing them to ‘slide back’ to their operating potential range.

The capacitor may be implemented in the circuitry of the medical device, such as the stimulation device 40 discussed above. The capacitor may hence be provided as a component on any of the PCBs 260 or provided separate from the PCB 260, discussed in further detail below.

FIG. 64G is a schematic illustration of a stimulation device 40 configured to deliver a signal in a vagal afferent 231 innervating an stomach tissue 230 of a patient. The stimulation device 40 may form part of a system comprising a sensor device 250 configured to generate a sensor signal indicating the response in the celiac vagus nerve, illustrated herein as part of the CNS 233, as well as a control unit, or controller 240, operable to receive the sensor signal and to control an operation of the stimulation device 40 based at least in part on the sensor signal. For illustrative purposes, the stimulation signal is in the present figure applied by means of an electrode arrangement 210 arranged to touch the nerve 231. However, the stimulation signal could just as well be applied to the stomach tissue 230 instead. The exemplary electrode arrangement 210 may be a unipolar electrode, comprising a first stimulation electrode 211 that may act as a cathode or anode. Another electrode (not shown) may be provided elsewhere to close the electric circuit.

The sensor device 250 may be configured to measure the neural response in various ways.

The sensor device 250 may be configured to employ one or more electrodes for measuring an electrical characteristic of the celiac vagus nerve.

FIG. 64H shows an example in which the sensor device 250 comprises one or more sensor electrodes 251, 252 configured to measure an electric activity in the celiac vagus nerve 233 in response to the electrical stimulation signal. This approach may be referred to as electromyography, EMG. The electrode(s) may be arranged to measure the electric activity in the celiac vagus nerve 233. An increased activity in the celiac vagus nerve 233 may indicate that a similar bodily response of the stimulation has been obtained as would have been obtained by distension of the stomach wall caused by eating. In the present example, a first sensor electrode 251 and a second sensor electrode 252 are provided to generate the sensor signal. The voltage signal, indicating the response in the celiac vagus nerve 233, may typically be in the range of 1-2 mV.

The sensor electrode 251-254 of the sensor device 250 may be configured to be arranged at the celiac vagus nerve 230 or inserted into the celiac vagus nerve 230. The sensor electrode(s) 251 may in some examples be formed as one or more patch electrodes that can be attached to the celiac vagus nerve 230. In some examples, the sensor electrode(s) 251 may be formed as needle electrodes arranged to protrude at least partially into the celiac vagus nerve 230.

The sensor device 250 may further comprise a reference electrode, allowing the sensor signal to be based on an electrical interaction between one or more sensor electrode 251 and the reference electrode. The reference electrode may be formed by a housing of the stimulation device 40, and/or an electrode arranged at the effector tissue 230, spaced apart from the sensor electrode 211.

As mentioned above, the sensor device 250 may in some examples comprise one or more mechanical sensor elements for measuring a mechanical characteristics or response. The sensor device 250 may, for example, be configured to measure mechanical movement in the effector tissue 230. FIG. 64J shows an example of such a sensor device 250, which comprises a strain gauge for measuring a contraction or relaxation of effector tissue 230 in response to the stimulation signal. Thus, the effector tissue 230 may be muscle tissue, such as smooth tissue. The strain may be positive (due to elongation of the muscle tissue) or negative (compressive, due to contraction of the muscle tissue). The strain gauge may be arranged to convert a change in dimension to a change in electrical resistance. In the present example, the strain gauge comprises a wire or foil 256 arranged in a grid pattern. During operation, the electrical resistance of the strain gauge may change in proportion to the deformation (and thus strain) experienced by the wire or foil pattern 256. An excitation voltage may be applied to the strain gauge and a sense voltage measured as an output voltage. As the resistance changes due to induced strain, the output voltage also changes.

The present example comprises a metallic foil pattern 256 arranged on a flexible support 255, such as a thin silicone film 255. The flexible support, or support patch 255, can be attached to an outer surface of the effector tissue 230 to be measured. Due to the flexible nature of the support 255, it may deform and contract as the effector tissue 230 deforms and contracts, thereby causing the metallic foil pattern 256 to deform accordingly.

The output from the sensor device 250 may be retrieved by the control unit 240, which may be configured to determine a response measure based on the sensor signal. The response measure may be understood as a measure indicative of the response in the celiac vagus nerve. Hence, the response measure may be a certain voltage, impedance, phase, resistance depending on the principle of operation used by the sensor device. In case of the sensor device 250 being an EMG sensor, the response measure may be a voltage, in case of an EIM sensor, the response measure may be an impedance and/or phase.

The control unit 240 may be operable to compare the response measure with a predetermined reference measure and to control the stimulation device based on the comparison to adjust or maintain a desired response in the effector tissue 230. The control unit 240 may, for example, increase an intensity of the stimulation signal in response to the response measure being below the reference measure and to reduce the intensity of the stimulation signal in response to the response measure exceeding the reference measure. The control unit 240 may thus operate as a closed-loop controller, or feedback controller, using information carried by the sensor signal as feedback when controlling the operation of the stimulation device 40 in a control loop. The control unit 240 may be configured to increase the intensity of the stimulation signal by increasing at least one of a frequency, current amplitude, and voltage amplitude of the stimulation signal. Further, the control unit 240 may be configured to reduce the intensity of the stimulation signal by reducing at least one of the frequency, current amplitude, and voltage amplitude of the stimulation signal.

The predetermined reference measure may be based on a previous measurement of the effector response in the patient, and/or on previous measurements of effector responses in other patients.

The control unit 240 may be configured to monitor the level of celiac vagus nerve over time, and to control the stimulation device based on a change rate in the effector response over time. Thus, the control unit 240 may be arranged to calculate a time derivative of the effector response and control the operation of the stimulation device 40 accordingly.

It will be appreciated that the response measure in some examples may be used to determine a calibration parameter of the stimulation device 40. The determination of the calibration parameter may form part of a calibration process, which may be performed in connection with implantation of the stimulation device 40. The calibration process may also be performed intermittently or on a regular basis, for example upon request by a healthcare professional. The calibration parameter may indicate an offset needed to adjust a characteristic of the stimulation signal, such as a voltage, frequency, or current, to achieve a desired level of effector response. The calibration process may hence be performed to ensure proper operation of the stimulation device 40 and increase the prospects of a desired and predictable effect of the applied stimulation signal.

A printed circuit board, PCB 260, may be employed to accommodate the circuitry and electrical components enabling the functionality of the system described above. Accordingly, at least one of the stimulation device 40, the source of energy, and the control unit 240 may supported by such as PCB. The PCB may be integrated in a housing or casing facilitating implantation in the body of the patient. Specific examples of PCBs 260 will now be discussed with reference to FIGS. 23K—23L.

The PCB 260 serves as a physical platform for supporting and interconnecting electronic components of the system. The PCB 260 typically comprises a substrate 263 on which conductive paths 261 are etched or printed to establish electrical connections. Components such as resistors, capacitors, and integrated circuits such as ASICs may then be mounted on the substrate 263. The design and configuration of the PCB 260 depend on the intended application and site of implantation, with considerations for size and flexibility playing roles.

FIG. 64K shows an example of a multi-layer PCB 260, in which a plurality of substrate layers 263, each provided with its own set of conductive paths 261, are bonded together with layers of insulation. The multi-layer configuration reduces the footprint of the PCB 260, allowing for more functionality within the limited space of a medical implant such as the stimulation device 40. The layers 263 are interconnected through vias 262, which may be through-holes filled or plated with conductive material. The relatively compact design of the PCB 260, with the reduced footprint enabled by the multi-layered configuration, makes it possible to implant the device in locations where the available space is relatively restricted.

The PCB 260 may also be of a flexible type, and or a stretchable type. Flexible PCBs are typically made using a flexible substrate, such polyimide or polyester film, which allows the PCB to conform to a specific shape or to flex during its use. This flexibility is particularly advantageous in medical implants that need to move of flex with the surrounding tissue, reducing the risk of damage to both the device and the tissue. Flexible PCBs can be single-layered or multi-layered and may, beneficially, be used in implants requiring adaptability to movement or specific anatomic contours.

Stretchable PCBs may be fabricated from materials that can withstand stretching, such as silicone-based substrates with conductive paths that can withstand stretching. The conductive paths may, for example, be formed of silver-filled silicone, or a conductive path or wire that is arranged in a ‘serpentine’ trace. The serpentine trace may be characterized by its zigzag or wave-like pattern, effectively distributing mechanical stress over a larger area and absorbing deformations caused by the substrate moving or stretching.

FIG. 64L shows a particular example of a PCB 260 which is both flexible and stretchable.

The PCB 260 comprises one or more flexible portions, such as flexible substrate portions 263, as well as one or more flexible portions comprising a stretchable substrate 264 with a conductive path 265 arranged in a wave-like pattern. In the present example, the PCB 260 comprises three flexible portions 263 interconnected by two stretchable substrate portions 264. The resulting arrangement is a PCB 260 that can conform to the specific shape of the tissue to which it is attached and adapt to movements such as contractions of the tissue. In further examples, two or more non-flexible portions (such as regular planar PCB portions or multi-layered PCB portions) may be interconnected by one or more flexible or stretchable portions to provide some flexibility/stretchability to the arrangement.

Various measures may be taken to ensure electrical safety and to comply with different regulatory frameworks. Direct current (DC) flowing through electrodes or other implanted parts of a system according to any of the aspects of the present disclosure may be a safety concern, as it may cause tissue damage. For example, it has been reported that DC levels as low as 2-3 μA may cause pathological changes in nerve tissue. It is therefore desirable to limit leakage current (DC) to 1 μA or less, such a 0.1 μA or less. This may be achieved by means of a capacitor, also referred to as a DC blocking capacitor, which may be arranged in any of the current pathways. Specifically, the capacitor may be connected in series with two or more electrodes of the implant, such as the ones employed to apply a stimulation signal or a measuring signal. Furthermore, the capacitor may be connected in series with a part of the implant (such as an electrode, an energy source, or a housing) and the body of the patient, thereby reducing any current that might flow between the implant and tissue of the patient.

A further advantage of the capacitor relates to prevention of charge accumulation on the electrodes. By coupling a capacitor to the electrodes, the capacitor may help dissipating accumulated charge from the electrodes, thereby allowing them to ‘slide back’ to their operating potential range.

The capacitor may be implemented in the circuitry of the medical device, such as the stimulation device 40 discussed above. The capacitor may hence be provided as a component on any of the PCBs 260 or provided separate from the PCB 260.

The present invention also relates to implantable vibration devices configured to deliver therapeutic or non-therapeutic vibration stimulation to human tissue at an implanted position in the human body. Implantable vibration devices which can be incorporated in any of the members described herein will now be described with reference to FIGS. 64M—64Q. More specifically, at least one implantable vibration device could be placed on or in a member of the embodiments shown in FIGS. 1A, 1B, 2A, 3A, 4A, 5, 6, 7, 8, 9, 11, 12A, 13A, 14A, 14B, 14C, 14D, 15, 17A, 21, 22, 23, 24, 25, 26, 27, 28A, 29A, 30A, 31a, 31C, 31D, 31F, 31G, 31H, 31I, 31J, 31K, 31K′, 31L, 31L′, 31M, 31N, 31O, 31P, 31Q-31T, 31U, 31V, 31X, 31Y, 33A-33H and 34A-341.

FIG. 64M schematically illustrates an implantable vibration device 1110 which is suitable for incorporation on or in a member for stretching the stomach or intestine. The implantable vibration device 1110 comprising a casing 1120. Contained in the casing 1120, a piezoelectric vibration generating unit VGU is provided. The VGU is capable of causing the implantable vibration device 1110 to vibrate in a suitable manner. In FIG. 64M, the implantable vibration device 1110 is also provided with wireless energy receiver R for receiving wireless energy for the operation of the vibration generating unit, and an internal controller CI configured to control the operation of the vibration generating unit. By providing a wireless energy receiver R and a controller CI within the casing 1120, a physically independent vibration generating device which can be implanted in the human body is obtained. Thus, leads and cables for controlling and/or providing energy to the vibration device can be omitted.

In alternative embodiments, the wireless energy receiver R and the internal controller may be provided in a different casing, separate from the casing 1120, and connected to the vibration generating unit via lead or cable. Preferably, such lead or cable flexible and short, typically having a length of less than 10 cm, such as of less than 5 cm. This may be advantageous as it allows for the casing 120 to be implanted via invagination in the tissue of the patient, whereas a smaller casing containing the wireless energy receiver and/or internal control CI can be implanted in a different manner.

The casing is shown in FIG. 64M to have a substantially cylindrical outer shapes, but other shapes such a sphere can also be contemplated, which is shown in FIG. 64N. The casing may be made of any suitable biocompatible material known to the person skilled in the art. In order to mitigate fibrin creation caused by contact between the implantable vibration device 1110 and the tissue or flowing blood of a patient, the implantable casing may comprise a specific coating arranged on the outer surface of the casing. The coating may comprise at least one layer of a biomaterial. The biomaterial is preferably fibrin-based. The coating may comprise at least one drug or substance with antithrombotic and/or antibacterial and/or antiplatelet characteristics. The drug or substance may be encapsulated in a porous material.

There may be provided a second coating arranged on the first coating. The second coating may be a different biomaterial than said first coating. In particular, the first coating may comprise a layer of perfluorocarbon chemically attached to the surface and the second coating may comprise a liquid perfluorocarbon layer.

Finally, the surface may comprise a micro pattern, wherein the micro pattern may be etched into the surface prior to insertion into the body. The layer of a biomaterial may be coated on the micro pattern.

According to an embodiment, implantable vibration device may be configured to be invaginated when placed on the outside of the stomach wall. In another embodiment, the movement restriction device may be configured to be invaginated when placed on the inside of the stomach wall.

In some embodiments, the implantable vibration device 1110 is configured to be at least partially invaginated by the tissue of the stomach wall using stomach-to-stomach sutures or staplers. In some embodiments, the system comprises the stomach-to-stomach sutures or staplers. Thus, the implantable vibration device can be kept in a partly invaginated position, preferably on the outside of the stomch or intestine wall. Consequently, the vibration device can be kept in a position where it abuts the tissue of the stomach wall.

The vibration device may be adapted to be placed in the stomach cavity. To this end, the vibration device may be adapted to be inserted into the stomach cavity via a gastroscope or intraluminar instrument, and be adapted to be invaginated in the stomach or intestine wall by surgery.

In some embodiments, the implantable vibration device is configured to abut the tissue of the stomach wall on the outside thereof, preferably be being invaginated by the tissue of the stomach wall.

In some embodiments the implantable vibration device is configured to abut the tissue of the intestine wall on the outside thereof, preferably be being invaginated by the tissue of the intestine wall.

In some embodiments, the implantable vibration device is configured to be implanted in or near the sexually responsive tissue near the vulva of a female patient.

In some embodiments, the implantable vibration device has a volume of from 0.3 cm3-6.6 cm3, or in the range 0.5 cm3-7.3 cm3, or in the range 3 cm3-8 cm3, or in the range 2.5 cm3-6.6 cm3, or in the range 4 cm3-7.3 cm3.

The wireless energy receiver R is configured to receive wireless energy for the operation of the vibration generating unit. In some embodiments, the wireless energy receiver R of the implantable vibration device includes a secondary coil, configured to receive wireless energy from a wireless energy transmitter, preferably comprising a primary coil configured to induce a voltage in the secondary coil of the vibration device. This way, energy can be transmitted wirelessly from the energy transmitter to the energy receiver via the primary and secondary coils. A suitable transmitter is typically implanted at a second, distant position in the body of the patient.

The vibration generating unit VGU is configured to cause the implantable vibrator to vibrate, so that it is capable of temporarily displacing tissue of the patient when the vibrator is in its implanted position in the body.

The vibrations provided by the vibration device can generally be defined by their frequency, their period and by their amplitude. The frequency denotes the number of complete cycles of vibration occurring per period of time.

The vibration generating unit VGU can be configured to cause the implantable vibrator to vibrate with a frequency in the range of from 0.01 Hz to 10,000 Hz.

It has been found that a vibration frequency in the range of 1-200 Hz, such as in the range of 1-150 Hz, such as in the range of 30-150 Hz, preferably in the range of 35-150 Hz, such as in the range of 35-100 hHz, such as in the range of 60-100 Hz has proven advantageous for activating at least some of the mechanoreceptors responsible for at least part of the control of satiety in the patient.

In some embodiments, the implantable vibration device is configured to vibrate with a period of 0.01-1 seconds, such as of 0.05-1 seconds. The period of the vibration is defined as the time it takes for the vibration to complete its cycle.

In some embodiments, vibration generating unit VGU is configured to cause the implantable vibrator 1110 to vibrate at an amplitude of at least 0.01 mm. In some embodiments the vibration generating unit VGU is configured to cause the implantable vibrator 1110 to vibrate at an amplitude of from 0.01 mm-30 mm.

The amplitude is defined as the maximum displacement of the mass center of the vibration device 110 from its resting position. Preferably, the vibration device 1110 is provided its invaginated position such that it can displace tissue of the stomach or intestine wall a distance approximately corresponding to the amplitude of the vibrations.

A displacement of tissue of at least 1 mm has been found sufficient to activate the relevant mechanoreceptors in the stomach.

Preferably, the implantable vibration device is configured to vibrate at an amplitude of at least 1 mm, such as of at least 2 mm, such as of at least 3 mm, such as at of at least 4 mm, such as at least 5 mm, such as at least 6 mm. In some embodiments, the vibration generating unit VGU is configured to cause the implantable vibrator 1110 to vibrate at an amplitude in the range of 1-10 mm, such in the range of 1-5 mm, preferably 2−4 mm.

This means that tissue which abuts the vibration device in the implanted position can displaced a corresponding length by each vibration cycle.

In some embodiments, the vibration device has a mass of at least 10 g. A sufficient mass is preferred such that a sufficient force can be delivered to the tissue for activating at least some of the mechanoreceptors.

The vibration generating unit is preferably operated by a piezoelectric motor. Depending on the piezoelectric motor type, the vibrations may be caused by various mechanisms, such as those exemplified in the following.

The piezoelectric effect is a property of certain solid materials to generate an electrical voltage in response to an applied mechanical stress (so-called direct piezoelectric effect) and to deform elastically in response to an applied electrical voltage (so-called inverse piezoelectric effect). The piezoelectric effect is a reversible process, meaning that materials exhibiting the direct piezoelectric effect also exhibit the inverse piezoelectric effect.

Materials exhibiting the piezoelectric effect are denoted as piezoelectric materials. Examples of piezoelectric materials comprise: crystalline materials, such as lithium niobate, lithium tantalate and quartz; ceramics, such as lead zirconate titanate, potassium niobate and barium titanate; polymers, such as polyvinylidene fluoride.

FIGS. 64O and 64P schematically shows an implantable vibration device 1110. FIGS. 64O and 64P differ in the shape of the casing 1120. FIG. 640 shows a substantially cylindrical casing.

FIG. 64P shows a substantially spherical casing.

The implantable vibration device comprises a casing 1110 which contains a vibration generating unit VGU. Herein, the vibration generating unit VGU is based on an eccentric mechanism for causing the implantable vibration device to vibrate. The vibration generating unit comprises motor 1604, a first motor axis 1606, an eccentric element 1608 eccentrically mounted to the first motor axis 1606, a second axis 1610 which suitably is supported by a bearing mounted to the casing 1120. The vibration generating unit VGU may comprise a gear box 1611 that transforms the speed of rotation of the motor 1604 to a suitable speed.

Upon operation of the motor 1604, the eccentric element 1608 will rotate eccentrically about the first axis 1606, to thereby cause the implantable vibration device to vibrate. The operation of the motor is preferably operated by the internal control unit C1. The motor is preferably powered by energy received wirelessly by the energy transmitter T.

Another option for causing vibrations in the implantable vibration device it to mount a weight on the motor 1604 via an axis 1606, wherein said axis is attached to the weight at a position offset from the center of the weight. Upon rotation of the weight, the implantable vibration device will be made to vibrate.

All parts of the vibration generating unit VGU, including the motor 1604 are preferably made of materials compatible with MR1 scanning. Consequently, the VGU does not comprise any metallic and/or magnetic parts, and can instead by manufactured by polymeric and/or ceramic materials.

FIG. 64Q illustrates an alternative mechanism suitable for a vibration generating unit, which is also based on a piezoelectric motor. In FIG. 64Q, there is a shown a schematic side view of vibration generating unit VGU, suitable to be placed in an implantable vibration device 1110 according to the present invention. FIG. 64Q shows a simple design of a vibration generating mechanism which comprises a piezoelectric material 1101 sandwiched between a respective electrode layers 1102a, b, preferably comprised of copper foils. Each electrode layer comprises a tab which extends outside the footprint of the piezoelectric material, which allows for further electrical connections in the form of leads connected to the wireless energy receiver R and/or controller C1. The energy transfer from the energy receiver R to the vibration generating unit is typically controlled by an internal controller C1.

The piezoelectric material 1101 is configured to extend in response to an applied electrical voltage controllable by the internal controller C1, or by an external controller. Once the voltage is removed or reversed, the piezoelectric material 1101 contracts to its relaxed state. Consequently, by alternatingly applying and removing (or reversing) a voltage over the piezoelectric material, the material can be made to expand and contract at a frequency which corresponds to the frequency of the alternating voltage. If this is performed at a certain frequency, the alternating expansion and contraction can cause an implantable vibration device to vibrate at a corresponding frequency.

The vibration generating unit is attached to the casing 1120 of the implantable vibration device 1110 via attachment means 1104, such that the expansion and contraction movement of the piezoelectric material in the vibration generating unit can be transferred to the casing and thereby cause the implantable vibration device 1110 to vibrate. The function and features of the controller comprised in the medical device (such as comprised in the main portion, connected to the main portion or comprised in a remote unit) for controlling the medical device will now described with reference to FIGS. 65A—65FH. The features of the controller described with reference to FIGS. 65A—65FH may be implemented and combined with any of the embodiments of medical devices (with and without remote units) disclosed herein. The features may for example be implemented in the controllers (300) shown and described with reference to FIGS. 1a-4c, 7, 9, 11, 12a, 13a, 14a, 14b, 14c, 14d, 32-36c, 52, 56-57h and 59c-59e.

Any controller 300 may comprise an internal computing unit, also called a processor or processing unit, and it may comprise a communication unit and implement methods for communication, including verification, authentication and encryption of data, as described in the following.

The controller may comprise a collection of communication related sub-units such as a wired transceiver, a wireless transceiver, energy storage unit, an energy receiver, a computing unit, a memory, or a feedback unit. The sub-units of the controller may cooperate with each other or operate independently with different purposes. The sub-units of the controller may inherit the prefix “internal”. This is to distinguish these sub-units from the sub-units of the external devices as similar sub-units may be present for both the implanted controller and the external devices. The sub-units of the external devices may similarly inherit the prefix “external”.

A wireless transceiver may comprise both a wireless transmitter and a wireless receiver. The wireless transceiver may also comprise a first wireless transceiver and a second wireless transceiver. In this case, the wireless transceiver may be part of a first communication system (using the first wireless transceiver) and a second communication system (using the second wireless transceiver).

In some embodiments, two communication systems may be implemented using a single wireless transceiver in e.g. the implant and a single wireless transceiver in e.g. an external device (i.e. one antenna at the implant and one antenna at the external device), but where for example the network protocol used for data transmission from the external device to the implant is different from the network protocol used for data transmission from the implant to the external device, thus achieving two separate communication systems.

Alternatively, the wireless transceiver may be referred to as either a wireless transmitter or a wireless receiver as not all embodiments of secure wireless communication discussed herein require two-way communication capability of the wireless transceiver. The wireless transceiver may transmit or receive wireless communication via wireless connections. The wireless transceiver may connect to both the implant and to external devices, i.e. devices not implanted in the patient.

The wireless connections may be based on radio frequency identification (RFID), near field charge (NFC), Bluetooth, Bluetooth low energy (BLE), or wireless local area network (WLAN). The wireless connections may further be based on mobile telecommunication regimes such as IG, 2G, 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

A wired transceiver may comprise both a wired transmitter and a wired receiver. The wording wired transceiver aims to distinguish between it and the wireless transceiver. It may generally be considered a conductive transceiver. The wired transceiver may transmit or receive conductive communication via conductive connections. Conductive connections may alternatively be referred to as electrical connections or as wired connections. The wording wired however, does not imply there needs to be a physical wire for conducting the communication. The body tissue of the patient may be considered as the wire. Conductive connection may use the body of the patient as a conductor. Conductive connections may still use ohmic conductors such as metals to at least some extent, and more specifically at the interface between the wired transceiver and the chosen conductor.

Communication, conductive or wireless may be understood as digital or analogue. In analogue communication, the message signal is in analogue form i.e., a continuous time signal. In digital communication, usually digital data i.e., discrete time signals containing information is transmitted.

The controller may comprise a sensation generator. A sensation generator is a device or unit that generates a sensation. The sensation generated may be configured to be experienceable by the patient such that the patient may take actions to authenticate a device, connection or communication. The sensation generator may be configured to generate a single sensation or a plurality of sensation components. The sensation or sensation components may comprise a vibration (e.g. a fixed frequency mechanical vibration), a sound (e.g. a superposition of fixed frequency mechanical vibrations), a photonic signal (e.g. a non-visible light pulse such as an infra-red pulse), a light signal (e.g. a visual light pulse), an electric signal (e.g. an electrical current pulse) or a heat signal (e.g. a thermal pulse). The sensation generator may be implanted, configured to be worn in contact with the skin of the patient or capable of creating sensation without being in physical contact with the patient, such as a beeping alarm.

The sensations generated by the sensation generator may be configured to be experienceable by a sensory function or a sense of the patient from the list of tactile, pressure, pain, heat, cold, taste, smell, sight, and hearing. Sensations may be generated of varying power or force as to adapt to sensory variations in the patient. Power or force may be increased gradually until the patient is able to experience the sensation. Variations in power or force may be controlled via feedback. Sensation strength or force may be configured to stay within safety margins. The sensation generator may be connected to the implant. The sensation generator may be comprised within the implant or be a separate unit. A motor, e.g. of the active device or unit of the implant, for controlling a physical function in the body of the patient may provide a secondary function as a sensation generator, generating a vibration or sound. Generation of vibrations or sounds of the motor MO may be achieved by operating the motor at specific frequencies. When functioning as to generate a sensation the motor MO may operate outside of its normal ranges for frequency controlling a physical function in the body. The power or force of the motor when operating to generate a sensation may also vary from its normal ranges for controlling a physical function in the body. The motor for use as an active device and a sensation generator could for example be an implantable brushless DC motor with integrated gear box, such as the motors provided by Maxon group or Dr. Fritz Faulhaber.

An external device is a device which is external to the patient in which the implant is implanted in. The external device may also be enumerated (first, second, third, etc.) to separate different external devices from each other. Two or more external devices may be connected by means of a wired or wireless communication as described above, for example through IP (internet protocol), or a local area network (LAN). The wired or wireless communication may take place using a standard network protocol such as any suitable IP protocol (IPv4, IPv6) or Wireless Local Area Network (IEEE 802.11), Bluetooth, NFC, NFMI, RFID etc. The wired or wireless communication may take place using a proprietary network protocol. Any external device may also be in communication with the implant using wired or wireless communication according to the above. Communication with implanted devices may be thus accomplished with a wired connection or with wireless radiofrequency (RF) telemetry, or near field magnetic induction (NFMI) technologies. Other methods of wireless communication may be used to communicate with implants, including optical and ultrasound. Alternatively, the concept of intrabody communication may be used for wireless communication, which uses the conductive properties of the body to transmit signals, i.e. conductive (capacitive or galvanic) communication with the implant. Means for conductive communication between an external device and an implant may also be called “electrical connection” between an external device and an implant. The conductive communication may be achieved by placing a conductive member of the external device in contact with the skin of the patient. By doing this, the external device and/or the implant may assure that it is in direct electrical connection with the other device. The concept relies on using the inherent conductive or electrical properties of a human body. Signals may preferably be configured to affect the body or body functions minimally. For conductive communication this may mean using low currents. A current may flow from an external device to an implant or vice versa. Also, for conductive communication, each device may have a transceiver portion for transmitting or receiving the current. These may comprise amplifiers for amplifying at least the received current. The current may contain or carry a signal which may carry e.g. an authentication input, implant operation instructions, or information pertaining to the operation of the implant.

Alternatively, conductive communication may be referred to as electrical or ohmic or resistive communication.

The conductive member may be an integrated part of the external device (e.g. in the surface of a smartwatch that is intended to be in contact with the wrist of the person wearing it), or it may be a separate device which can be connected to the external device using a conductive interrace such as the charging port or the headphone port of a smartphone.

A conductive member may be considered any device or structure set up for data communication with the implant via electric conductive body tissue. The data communication to the implant may be achieved by e.g. current pulses transmitted from the conductive member through the body of the patient to be received by a receiver at the implant. Any suitable coding scheme known in the art may be employed. The conductive member may comprise an energy storage unit such as a battery or receive energy from e.g. a connected external device.

The term conductive interface is representing any suitable interface configured for data exchange between the conductive member and the external device. The conductive member may in an alternative configuration receive and transmit data to the external device through a radio interface, NFC, and the like.

An external device may act as a relay for communication between an implant and a remote device, such as e.g. second, third, or other external devices. Generally, the methods of relaying communication via an external device may be preferable for a large number of reasons. The transmission capabilities of the implant may be reduced, reducing its technical complexity, physical dimensions, and medical effects on the patient in which the implant is implanted. Communication may also be more efficient as direct communication, i.e. without a relaying device, with an implant from a remote device may require higher energy transmissions to account for different mediums and different rates of attenuation for different communication means. Remote communication with lower transmission energy may also increase the security of the communication as the spatial area or volume where the communication may be at all noticeable may be made smaller. Utilizing such a relay system further enables the use of different communication means for communication with the implant and communication with remote devices that are more optimized for their respective mediums.

An external device may be any device having processing power or a processor to perform the methods and functions needed to provide safe operation of the implant and provide the patient or other stakeholders (caregiver, spouse, employer etc.) with information and feedback from the implant. Feedback parameters could include battery status, energy level at the controller, the fluid level of the hydraulic restriction device, number of operations that the restriction device has performed, properties, version number etc. relating to functionality of the implantable medical device. The external device may for example be a handset such as a smartphone, smartwatch, tablet etc. handled by the patient or other stakeholders. The external device may be a server or personal computer handled by the patient or other stakeholders. The external device may be cloud based or a virtual machine. In the drawings, the external device handled by the patient is often shown as a smart watch, or a device adapted to be worn by the patient at the wrist of the patient. This is merely by way of example and any other type of external device, depending on the context, is equally applicable.

Several external devices may exist such as a second external device, a third external device, or another external device. The above listed external devices may e.g. be available to and controllable by a patient, in which an implant is implanted, a caregiver of the patient, a healthcare professional of the patient, a trusted relative of the patient, an employer or professional superior of the patient, a supplier or producer of the implant or its related features. By controlling the external devices may provide options for e.g. controlling or safeguarding a function of the implant, monitoring the function of the implant, monitoring parameters of the patient, updating or amending software of the implant etc.

An external device under control by a supplier or producer of the implant may be connected to a database comprising data pertaining to control program updates and/or instructions. Such database may be regularly updated to provide new or improved functionality of the implant, or to mitigate for previously undetected flaws of the implant. When an update of a control program of an implant is scheduled, the updated control program may be transmitted from the database in a push mode and optionally routed via one or more further external devices before received by the implanted controller. In another embodiment, the update is received from the database by request from e.g. an external device under control by the patient having the implant implanted in his/her body, a pull mode.

The external device may require authentication to be operated in communication with other external devices or the implant. Passwords, multi-factor authentication, biometric identification (fingerprint, iris scanner, facial recognition, etc.) or any other way of authentication may be employed.

The external device may have a user interface (UI) for receiving input and displaying information/feedback from/to a user. The UI may be a graphical UI (GUI), a voice command interface, speaker, vibrators, lamps, etc.

The communication between external devices, or between an external device and the implant may be encrypted. Any suitable type of encryption may be employed such as symmetric or asymmetric encryption. The encryption may be a single key encryption or a multi-key encryption. In multi-key encryption, several keys are required to decrypt encrypted data. The several keys may be called first key, second key, third key, etc. or first part of a key, second part of the key, third part of the key, etc. The several keys are then combined in any suitable way (depending on the encryption method and use case) to derive a combined key which may be used for decryption. In some cases, deriving a combined key is intended to mean that each key is used one by one to decrypt data, and that the decrypted data is achieved when using the final key.

In other cases, the combination of the several key result in one “master key” which will decrypt the data. In other words, it is a form of secret sharing, where a secret is divided into parts, giving each participant (external device(s), internal device) its own unique part. To reconstruct the original message (decrypt), a minimum number of parts (keys) is required. In a threshold scheme this number is less than the total number of parts (e.g. the key at the implant and the key from one of the two external device are needed to decrypt the data). In other embodiments, all keys are needed to reconstruct the original secret, to achieve the combined key which may decrypt the data.

In should be noted that it is not necessary that the generator of a key for decryption is the unit that in the end sends the key to another unit to be used at that unit. In some cases, the generator of a key is merely a facilitator of encryption/decryption, and the working in behalf of another device/user.

A verification unit may comprise any suitable means for verifying or authenticating the use (i.e. user authentication) of a unit comprising or connected to the verification unit, e.g. the external device. For example, a verification unit may comprise or be connected to an interface (UI, GUI) for receiving authentication input from a user. The verification unit may comprise a communication interface for receiving authentication data from a device (separate from the external device) connected to the device comprising the verification unit. Authentication input/data may comprise a code, a key, biometric data based on any suitable techniques such as fingerprint, a palm vein structure, image recognition, face recognition, iris recognition, a retinal scan, a hand geometry, and genome comparison, etc. The verification/authentication may be provided using third party applications, installed at or in connection with the verification unit.

The verification unit may be used as one part of a two-part authentication procedure. The other part may e.g. comprise conductive communication authentication, sensation authentication, or parameter authentication.

The verification unit may comprise a card reader for reading a smart card. A smart card is a secure microcontroller that is typically used for generating, storing and operating on cryptographic keys. Smart card authentication provides users with smart card devices for the purpose of authentication. Users connect their smart card to the verification unit. Software on the verification unit interacts with the keys material and other secrets stored on the smart card to authenticate the user. In order for the smart card to operate, a user may need to unlock it with a user-PIN. Smart cards are considered a very strong form of authentication because cryptographic keys and other secrets stored on the card are very well protected both physically and logically, and are therefore hard to steal.

The verification unit may comprise a personal e-ID that is comparable to, for example, passport and driving license. The e-ID system comprises is a security software installed at the verification unit, and a e-ID which is downloaded from a web site of a trusted provided or provided via a smart card from the trusted provider.

The verification unit may comprise software for SMS-based two-factor authentication. Any other two-factor authentication systems may be used. Two-factor authentication requires two things to get authorized: something you know (your password, code, etc.) and something you have (an additional security code from your mobile device (e.g. a SMS, or a e-ID) or a physical token such as a smart card).

Other types of verification/user authentication may be employed. For example, a verification unit which communicate with an external device using visible light instead of wired communication or wireless communication using radio. A light source of the verification unit may transmit (e.g. by flashing in different patterns) secret keys or similar to the external device which uses the received data to verify the user, decrypt data or by any other means perform authentication. Light is easier to block and hide from an eavesdropping adversary than radio waves, which thus provides an advantage in this context. In similar embodiments, electromagnetic radiation is used instead of visible light for transmitting verification data to the external device.

Parameters relating to functionality of the implant may comprise for example a status indicator of the implant such as battery level, version of control program, properties of the implant, status of a motor of the implant, etc.

Data comprising operating instructions sent to the implant may comprise a new or updated control program, parameters relating to specific configurations of the implant, etc. Such data may for example comprise instructions how to operate the body engaging portion of the implantable medical device, instructions to collect patient data, instructions to transmit feedback, etc.

The expressions “confirming the electrical connection between an implant and an external device” or “authenticating a connection between an implant and an external device”, or similar expressions, are intended to encompass methods and processes for ensuring or be reasonably sure that the connection has not been compromised. Due to weaknesses in the wireless communication protocols, it is a simple task for a device to “listen” to the data and grab sensitive information, e.g. personal data regarding the patient sent from the implant, or even to try to compromise (hack) the implant by sending malicious commands or data to the implant. Encryption may not always be enough as a security measure (encryption schemes may be predictable), and other means of confirming or authenticating the external device being connected to the implant may be needed.

The expression “network protocol” is intended to encompass communication protocols used in computer networks. a communication protocol is a system of rules that allow two or more entities of a communications system to transmit information via any kind of variation of a physical quantity. The protocol defines the rules, syntax, semantics and synchronization of communication and possible error recovery methods. Protocols may be implemented by hardware, software, or a combination of both. Communication protocols have to be agreed upon by the parties involved. In this field, the term “standard” and “proprietary” is well defined. A communication protocol may be developed into a protocol standard by getting the approval of a standards organization. To get the approval the paper draft needs to enter and successfully complete the standardization process. When this is done, the network protocol can be referred to a “standard network protocol” or a “standard communication protocol”. Standard protocols are agreed and accepted by whole industry. Standard protocols are not vendor specific. Standard protocols are often, as mentioned above, developed by collaborative effort of experts from different organizations.

Proprietary network protocols, on the other hand, are usually developed by a single company for the devices (or Operating System) which they manufacture. A proprietary network protocol is a communications protocol owned by a single organization or individual. Specifications for proprietary protocols may or may not be published, and implementations are not freely distributed. Consequently, any device may not communicate with another device using a proprietary network protocol, without having the license to use the proprietary network protocol, and knowledge of the specifications for proprietary protocol. Ownership by a single organization thus gives the owner the ability to place restrictions on the use of the protocol and to change the protocol unilaterally.

A control program is intended to define any software used for controlling the implant. Such software may comprise an operating system of the implant, of parts of an operating system or an application running on the implant such as software controlling a specific functionality of the implant (e.g. the active unit of the implant, feedback functionality of the implant, a transceiver of the implant, encoding/decoding functionality of the implant, etc.). The control program may thus control the medical function of the implant, for example the pressure applied by a member or the power of the electrical stimulation device. Alternatively or additionally, the control program may control internal hardware functionality of the implant such as energy usage, transceiver functionality, etc.

The systems and methods disclosed hereinabove may be implemented as software, firmware, hardware or a combination thereof. In a hardware implementation, the division of tasks between functional units referred to in the above description does not necessarily correspond to the division into physical units; to the contrary, one physical component may have multiple functionalities, and one task may be carried out by several physical components in cooperation. Certain components or all components may be implemented as software executed by a digital signal processor or microprocessor or be implemented as hardware or as an application-specific integrated circuit. Such software may be distributed on computer readable media, which may comprise computer storage media (or non-transitory media) and communication media (or transitory media). As is well known to a person skilled in the art, the term computer storage media includes both volatile and non-volatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information, and which can be accessed by a computer. Further, it is well known to the skilled person that communication media typically embodies computer readable instructions, data structures, program modules or other data in a modulated data signal such as a carrier wave or other transport mechanism and includes any information delivery media.

A controller 300 for controlling the medical device according to any of the embodiments herein and for communicating with devices external to the body of the patient and/or implantable sensors will now be described with reference to FIGS. 65A—65C. FIG. 65A shows a patient when a medical device 10 comprising a controller 300 has been implanted. The medical device 10 comprises an active unit 302, which is the part of the medical device which comprises the one or more members and operation device for operating the members etc.. The active unit is directly or indirectly connected to the stomach wall of the patient for stretching the stomach wall for creating a sensation of satiety. The active unit 302 is connected to the controller 300 via an electrical connection C2. The controller 300 (further described with reference to FIG. 65b) is configured to communicate with an external device 320 (further described with reference to FIG. 65c). The controller 300 can communicate wirelessly with the external device 320 through a wireless connection WL1, and/or through an electrical connection C1.

Referring now to FIG. 65b, one embodiment of the controller 300 will be describe in more detail. The controller 300 comprises an internal computing unit 306 configured to control the function performed by the implantable medical device 10. The computing unit 306 comprises an internal memory 307 configured to store programs thereon. In the embodiment described in FIG. 65b, the internal memory 307 comprises a first control program 310 which can control the function of the medical device 10. The first control program 310 may be seen as a program with minimum functionality to be run at the medical device only during updating of the second control program 312. When the medical device is running with the first control program 310, the medical device may be seen as running in safe mode, with reduced functionality. For example, the first control program 310 may result in that no sensor data is stored in the medical device while being run, or that no feedback is transmitted from the medical device while the first control program 310 is running. By having a low complexity first control program, memory at the medical device is saved, and the risk of failure of the medical device during updating of the second control program 312 is reduced.

The second control program 312 is the program controlling the medical device in normal circumstances, providing the medical device with full functionality and features.

The memory 307 can further comprise a second, updatable, control program 312. The term updatable is to be interpreted as the program being configured to receive incremental or iterative updates to its code, or be replaced by a new version of the code. Updates may provide new and/or improved functionality to the implant as well as fixing previous deficiencies in the code. The computing unit 306 can receive updates to the second control program 312 via the controller 300. The updates can be received wirelessly WL1 or via the electrical connection C1. As shown in FIG. 65b, the internal memory 307 of the controller 300 can possibly store a third program 314. The third program 314 can control the function of the implantable medical device 10 and the computing unit 306 may be configured to update the second program 312 to the third program 314. The third program 314 can be utilized when rebooting an original state of the second program 312. The third program 314 may thus be seen as providing a factory reset of the controller 300, e.g. restore it back to factory settings. The third program 314 may thus be included in the implant 300 in a secure part of the memory 307 to be used for resetting the software (second control program 312) found in the controller 300 to original manufacturer settings.

The controller 300 may comprise a reset function 316 connected to or part of the internal computing unit 306 or transmitted to said internal computing unit 306. The reset function 316 is configured to make the internal computing unit 306 switch from running the second control program 312 to the first control program 310. The reset function 316 could be configured to make the internal computing unit 306 delete the second control program 312 from the memory 307. The reset function 316 can be operated by palpating or pushing/put pressure on the skin of the patient. This could be performed by having a button on the implant. Alternatively, the reset function 316 can be invoked via a timer or a reset module. Temperature sensors and/or pressure sensors can be utilized for sensing the palpating. The reset function 316 could also be operated by penetrating the skin of the patient. It is further plausible that the reset function 316 can be operated by magnetic means. This could be performed by utilizing a magnetic sensor and applying a magnetic force from outside the body. The reset function 316 could be configured such that it only responds to magnetic forces applied for a duration of time exceeding a limit, such as 2 seconds. The time limit could equally plausible be 5 or 10 seconds, or longer. In these cases, the implant could comprise a timer. The reset function 316 may thus include or be connected to a sensor for sensing such magnetic force.

In addition to or as an alternative to the reset function described above, the implant may comprise an internal computing unit 306 (comprising an internal processor) comprising the second control program 312 for controlling a function of the implantable medical device, and a reset function 318. The reset function 318 may be configured to restart or reset said second control program 312 in response to: i. a timer of the reset function 318 has not been reset, or ii. a malfunction in the first control program 310.

The reset function 318 may comprise a first reset function, such as, for example, comprise a computer operating properly, COP, function connected to the internal computing unit 306. The first reset function may be configured to restart or reset the first or the second control program 312 using a second reset function. The first reset function comprises a timer, and the first or the second control program is configured to periodically reset the timer.

The reset function 318 may further comprise a third reset function connected to the internal computing unit and to the second reset function. The third reset function may in an example be configured to trigger a corrective function for correcting the first 310 or second control program 312, and the second reset function is configured to restart the first 310 or second control program 312 sometime after the corrective function has been triggered. The corrective function may be a soft reset or a hard reset.

The second or third reset function may, for example, configured to invoke a hardware reset by triggering a hardware reset by activating an internal or external pulse generator which is configured to create a reset pulse. Alternatively, the second or third reset function may be implemented by software.

The controller 300 may further comprise an internal wireless transceiver 308. The transceiver 308 communicates wirelessly with the external device 320 through the wireless connection W1. The transceiver may further communicate with an external device 320, 300 via wireless connection WL2 or WL4. The transceiver may both transmit and receive data via either of the connections C1, WL1, WL2 and WL4. Optionally, the external devices 320 and 300, when present, may communicate with each other, for example via a wireless connection WL3.

The controller 300 can further be electrically connected C1 to the external device 320 and communicate by using the patient's body as a conductor. The controller 300 may thus comprise a wired transceiver 303 or an internal transceiver 303 for the electrical connection C1.

The confirmation/authentication of the electrical connection can be performed as described herein in the section for confirmation and/or authentication. In these cases, the implanted medical device and/or external device(s) 320 comprises the necessary features and functionality (described in the respective sections of this document) for performing such confirmation/authentication. By authenticating according to these aspects, security of the authentication may be increased as it may require a malicious third party to know or gain access to either the transient physiological parameter of the patient or detect randomized sensations generated at or within the patient.

In FIGS. 65a-65c the patient is a human, but other mammals are equally plausible. It is also plausible that the communication is performed by inductive means. It is also plausible that the communication is direct.

The controller 300 of the implantable medical device 10 according to FIG. 65b further comprises a feedback unit 349. The feedback unit 349 provides feedback related to the switching from the second control program 312 to the first control program 310. The feedback could for example represent the information on when the update of the software, i.e. the second control program 312, has started, and when the update has finished. This feedback can be visually communicated to the patient, via for example a display on the external device 320. This display could be located on a watch, or a phone, or any other external device 320 coupled to the controller 300. Preferably, the feedback unit 349 provides this feedback signal wirelessly WL1 to the external device 320. Potentially, the words “Update started”, or “Update finished”, could be displayed to the patient, or similar terms with the same meaning. Another option could be to display different colors, where green for example could mean that the update has finished, and red or yellow that the update is ongoing. Obviously, any color is equally plausible, and the user could choose these depending on personal preference. Another possibility would be to flash a light on the external device 320. In this case the external device 320 comprises the light emitting device(s) needed. Such light could for example be a LED. Different colors could, again, represent the status of the program update. One way of representing that the update is ongoing and not yet finished could be to flash the light, i.e. turning the light on and off. Once the light stops flashing, the patient would be aware of that the update is finished. The feedback could also be audible, and provided by the implantable medical device 300 directly, or by the external device 320. In such cases, the implantable medical device 10 and external device 320 comprises means for providing audio. The feedback could also be tactile, for example in the form of a vibration that the user can sense. In such case, either the implantable medical device 10 or external device 320 comprises means for providing a tactile sensation, such as a vibration and/or a vibrator.

As seen in FIG. 65b, the controller 300 can further comprise a first energy storage unit 40A.

The first energy storage unit 40A runs the first control program 310. The controller 300 further comprises a second energy storage unit 40B which runs the second control program 312. This may further increase security during update, since the first control program 310 has its own separate energy storage unit 40A. The energy storage unit 40A can comprise a first energy storage 304a and/or a first energy receiver 305a. The second energy storage unit 40B can comprise a second energy storage 304b and/or a second energy receiver 305b. The energy can be received wirelessly by inductive or conductive means. An external energy storage unit can for example transfer an amount of wireless energy to the energy receiver 305a, 305b inside the patient's body by utilizing an external coil which induces a voltage in an internal coil (not shown in figures). It is plausible that the first energy receiver 305a receives energy via a RFID pulse. The feedback unit 349 can provide feedback pertaining to the amount of energy received via the RFID pulse. The amount of RFID pulse energy that is being received can be adjusted based on the feedback, such that the pulse frequency is successively raised until a satisfying level is reached.

The controller 300 of the medical device 10 according to FIG. 65b further comprises a feedback unit an electrical switch 309. The electrical switch 309 could be mechanically connected to a member of the medical device configured to exert a force on the stomach wall of the patient and being configured to be switched as a result of the force exerted on the stomach wall of the patient exceeding a threshold value. The switch 309 could for example be bonded to one of the members being connected to the stomach wall, in any of the embodiments herein, or to a portion of a fluid conduit, reservoir or hydraulic operation device, such as a pump, being in fluid connection with the member and be switched by the expansion, movement or bending of the member. The switch 309 could alternatively be electrically connected to the operation device and being configured to be switched as a result of the current supplied to the operation device exceeding a threshold value. The switch 309 could for example be connected to the motor and be configured to be switched if the current to the motor exceeds a threshold value. Such a switch could for example be a switch 309 configured to switch if exposed to a temperature exceeding a threshold value, such as a bimetal switch which is switched by the heat created by the flow of current to e.g. the motor. In the alternative, the switch 309 configured to switch if exposed to a temperature exceeding a threshold value could be placed at a different location on the medical device 10 to switch in case of exceeding temperatures, thereby hindering the medical device from overheating which may cause tissue damage.

The switch 309 could either be configured to cut the power to the operation device or to generate a control signal to the processor 306 of the implantable controller 300, such that the controller 300 can take appropriate action, such as reducing power or turning off the operation device.

The external device 320 is represented in FIG. 65c. The external device 320 can be placed anywhere on the patient's body, preferably on a convenient and comfortable place. The external device 320 could be a wristband, and/or have the shape of a watch. It is also plausible that the external device is a mobile phone or other device not attached directly to the patient. The external device as shown in FIG. 65c comprises a wired transceiver 323, and an energy storage 324. It also comprises a wireless transceiver 328 and an energy transmitter 325. It further comprises a computing unit 326 and a memory 327. The feedback unit 322 in the external device 320 is configured to provide feedback related to the computing unit 326. The feedback provided by the feedback unit 322 could be visual. The external device 320 could have a display showing such visual feedback to the patient. It is equally plausible that the feedback is audible, and that the external device 320 comprises means for providing audio. The feedback given by the feedback unit 322 could also be tactile, such as vibrating. The feedback could also be provided in the form of a wireless signal WL1, WL2, WL3, WL4.

The second, third or fourth communication methods WL2, WL3, WL4 may be a wireless form of communication. The second, third or fourth communication method WL2, WL3, WL4 may preferably be a form of electromagnetic or radio-based communication. The second, third and fourth communication method WL2, WL3, WL4 may be based on telecommunication methods. The second, third or fourth communication method WL2, WL3, WL4 may comprise or be related to the items of the following list: Wireless Local Area Network (WLAN), Bluetooth, Bluetooth 5, BLE, GSM or 2G (2nd generation cellular technology), 3G, 4G or 5G.

The external device 320 may be adapted to be in electrical connection C1 with the medical device 10, using the body as a conductor. The electrical connection C1 is in this case used for conductive communication between the external device 320 and the medical device 10.

In one embodiment, the communication between controller 300 and the external device 320 over either of the communication methods WL2, WL3, WL4, C1 may be encrypted and/or decrypted with public and/or private keys, now described with reference to FIGS. 65a-65c. For example, the controller 300 may comprise a private key and a corresponding public key, and the external device 320 may comprise a private and a corresponding public key.

The controller 320 and the external device 320 may exchange public keys and the communication may thus be performed using public key encryption. The person skilled in the art may utilize any known method for exchanging the keys.

The controller may encrypt data to be sent to the external device 320 using a public key corresponding to the external device 320. The encrypted data may be transmitted over a wired, wireless or electrical communication channel C1, WL1, WL2, WL3 to the external device. The external device 320 may receive the encrypted data and decode it using the private key comprised in the external device 320, the private key corresponding to the public key with which the data has been encrypted. The external device 320 may transmit encrypted data to the controller 300. The external device 320 may encrypt the data to be sent using a public key corresponding to the private key of the controller 300. The external device 320 may transmit the encrypted data over a wired, wireless or electrical connection C1, WL1, WL2, WL3, WL4, directly or indirectly, to the controller of the implant. The controller may receive the data and decode it using the private key comprised in the controller 300.

In an alternative to the public key encryption, described with reference to FIGS. 65a-65c, the data to be sent between the controller 300 of the implantable medical device 10 and an external device 320, 330 or between an external device 320, 330 and the controller 300 may be signed. In a method for sending data from the controller 300 to the external device 320, 330, the data to be sent from the controller 300 may be signed using the private key of the controller 300. The data may be transmitted over a communication channel or connection C1, WL1, WL2, WL3, WL4. The external device 320, 330 may receive the message and verify the authenticity of the data using the public key corresponding to the private key of the controller 300. In this way, the external device 320, 330 may determine that the sender of the data was sent from the controller 300 and not from another device or source.

A method for communication between external devices and the controller 300 of the implantable medical device 10 using a combined key is now described with reference to FIGS. 65a-65c. A first step of the method comprises receiving, at the implant, by a wireless transmission WL1, WL2, WL3, WL4 or otherwise, a first key from an external device 320, 330. The method further comprises receiving, at the implant, by a wireless transmission WL1, WL2, WL3, a second key. The second key may be generated by a second external device, separate from the external device 320, 330 or by another external device being a generator of the second key on behalf of the second external device 320, 330.

The second key may be received at the medical device from anyone of, the external device 320, the second external device 330, and the generator of the second key. The second external device may be controlled by a caretaker, or any other stakeholder. Said another external device may be controlled by a manufacturer of the implant, or medical staff, caretaker, etc.

In case the controller 300 is receiving the second key from the external device 320, this means that the second key is routed through the external device from the second external device 330 or from another external device (generator). The routing may be performed as described herein under the tenth aspect. In these cases, the implanted medical device and/or external device(s) comprises the necessary features and functionality (described in the respective sections of this document) for performing such routing. Using the external device 320 as a relay, with or without verification from the patient, may provide an extra layer of security as the external device 320 may not need to store or otherwise handle decrypted information. As such, the external device 320 may be lost without losing decrypted information. The controller 300 a computing unit 306 configured for deriving a combined key by combining the first key and the second key with a third key held by the controller 300, for example in memory 307 of the controller 300. The third key could for example be a license number of the implant or a chip number of the implantable medical device. The combined key may be used for decrypting, by the computing unit 306, encrypted data transmitted by a wireless transmission WL1 from the external device 320 to the controller 300. Optionally, the decrypted data may be used for altering, by the computing unit 306 an operation of the implantable medical device. The altering an operation of the implantable medical device may comprise controlling or switching an active unit 302 of the implantable medical device. In some embodiments, the method further comprises at least one of the steps of, based on the decrypted data, updating a control program running in the controller 300, and operating the implantable medical device 10 using operation instructions in the decrypted data.

Methods for encrypted communication between an external device 320 and the controller 300 are provided. These methods comprise:

receiving, at the external device 320, by a wireless transceiver 328, a first key, the first key being generated by a second external device 330, separate from the external device 320 or by another external device being a generator of the second key on behalf of the second external device 330, the first key being received from anyone of the second external device 330 and the generator of the second key,

receiving, at the external device 320 by the wireless transceiver 328, a second key from the controller 300,

deriving a combined key, by a computing unit 326 of the external device 320, by combining the first key and the second key with a third key held by the external device 320 (e.g. in memory 307),

transmitting encrypted data from the implant to the external device and receiving the encrypted data at the external device by the wireless transceiver 328, and

decrypting, by the computing unit 326, the encrypted data, in the external device 320, using the combined key.

As described above, further keys may be necessary to decrypt the data. Consequently, the wireless transceiver 328 is configured for:

receiving a fourth key from a third external device,

wherein the computing unit 326 is configured for:

deriving a combined key by combining the first, second and fourth key with the third key held by the external device, and

decrypting the encrypted data using the combined key.

These embodiments further increase the security in the communication. The computing unit 326 may be configured to confirm the communication between the implantable medical device and the external device, wherein the confirmation comprises:

measuring a parameter of the patient, by the external device 320,

receiving a measured parameter of the patient, from the implantable medical device 10,

comparing the parameter measured by the implantable medical device 10 to the parameter measured by the external device 320,

performing confirmation of the connection based on the comparison, and

as a result of the confirmation, decrypting the encrypted data, in the external device, using the combined key.

The keys described in this section may in some embodiments be generated based on data sensed by sensors described herein under the twelfth or thirteenth aspect, e.g. using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

Further, increased security for communication between an external device(s) and the implantable medical device is provided.

A method of communication between an external device 320 and an implantable medical device 10 is now described with reference to FIGS. 65a-65c, when the implantable medical device 10 is implanted in a patient and the external device 320 is positioned external to the body of the patient. The external device 320 is adapted to be in electrical connection C1 with the controller 300, using the body as a conductor. The electrical connection C1 is used for conductive communication between the external device 320 and the implantable medical device 10. The implantable medical device 10 comprises the controller 300. Both the controller 300 and the external device 320 comprises a wireless transceiver 308, 208 for wireless communication C1 between the controller 300 and the external device 320. The wireless transceiver 308 (included in the controller 300) may in some embodiments comprise sub-transceivers for receiving data from the external device 320 and other external devices, e.g. using different frequency bands, modulation schemes etc.

In a first step of the method, the electrical connection C1 between the controller 300 and the external device 320 is confirmed and thus authenticated. The confirmation and authentication of the electrical connection may be performed as described herein under the fifth, thirteenth and fifteenth aspect. In these cases, the implant and/or external device(s) comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

The implant may comprise a first transceiver 303 configured to be in electrical connection C1 with the external device, using the body as a conductor. The implantable medical device may comprise a first external transmitter 203 configured to be in electrical connection C1 with the implantable medical device, using the body as a conductor, and the wireless transmitter 208 configured to transmit wireless communication W1 to the controller 300. The first transmitter 323 of the external device 320 may be wired or wireless. The first transmitter 323 and the wireless transmitter 208 may be the same or separate transmitters. The first transceiver 303 of the controller 300 may be wired or wireless. The first transceiver 303 and the wireless transceiver 102 may be the same or separate transceivers.

The controller 300 may comprise a computing unit 306 configured to confirm the electrical connection between the external device 320 and the internal transceiver 303 and accept wireless communication WL1 (of the data) from the external device 320 on the basis of the confirmation.

Data is transmitted from the external device 320 to the controller 300 wirelessly, e.g. using the respective wireless transceiver 308, 208 of the controller 300 and the external device 320. Data may alternatively be transmitted through the electrical connection C1. As a result of the confirmation, the received data may be used for instructing the implantable medical device 10. For example, a control program 310 running in the controller 300 may be updated, the controller 300 may be operated using operation instructions in the received data. This may be handled by the computing unit 306.

The method may comprise transmitting data from the external device 320 to the controller 300 wirelessly comprises transmitting encrypted data wirelessly. To decrypt the encrypted data (for example using the computing unit 306), several methods may be used.

In one embodiment, a key is transmitted using the confirmed conductive communication channel C1 (i.e. the electrical connection) from the external device 320 to the controller 300. The key is received at the controller (by the first internal transceiver 303). The key is then used for decrypting the encrypted data.

In some embodiments the key is enough to decrypt the encrypted data. In other embodiments, further keys are necessary to decrypt the data. In one embodiment, a key is transmitted using the confirmed conductive communication channel C1 (i.e. the electrical connection) from the external device 320 to the controller 300. The key is received at the controller 300 (by the first internal transceiver 303). A second key is transmitted (by the wireless transceiver 208) from the external device 320 using the wireless communication WL1 and received at the controller 300 by the wireless transceiver 308. The computing unit 306 is then deriving a combined key from the key and second key and uses this for decrypting the encrypted data.

In yet other embodiments, a key is transmitted using the confirmed conductive communication channel C1 (i.e. the electrical connection) from the external device 320 to the controller 300. The key is received at the controller (by the first internal transceiver 303). A third key is transmitted from a second external device 330, separate from the external device 320, to the implant wirelessly WL2.

The third key may be received by a second wireless receiver (part of the wireless transceiver 308) of the controller 300 configured for receiving wireless communication WL2 from second external device 330.

The first and third key may be used to derive a combined key by the computing unit 306, which then decrypts the encrypted data. The decrypted data is then used for instructing the implantable medical device 10 as described above.

The second external device 330 may be controlled by for example a caregiver, to further increase security and validity of data sent and decrypted by the controller 300.

It should be noted that in some embodiments, the external device is further configured to receive WL2 secondary wireless communication from the second external device 330, and transmit data received from the secondary wireless communication WL2 to the implantable medical device. This routing of data may be achieved using the wireless transceivers 308, 208 (i.e. the wireless connection WL1, or by using a further wireless connection WL4 between the controller 300 and the external device 320. In these cases, the medical device and/or external device(s) comprises the necessary features and functionality for performing such routing. Consequently, in some embodiments, the third key is generated by the second external device 330 and transmitted WL2 to the external device 320 which routes the third key to the controller 300 to be used for decryption of the encrypted data. In other words, the step of transmitting a third key from a second external device, separate from the external device, to the implant wirelessly, comprises routing the third key through the external device 320. Using the external device 320 as a relay, with or without verification from the patient, may provide an extra layer of security as the external device 320 may not need to store or otherwise handle decrypted information.

As such, the external device 320 may be lost without losing decrypted information.

In yet other embodiments, a key is transmitted using the confirmed conductive communication channel C1 (i.e. the electrical connection) from the external device 320 to the controller 300. The key is received at the implant (by the first internal transceiver 303). A second key is transmitted from the external device 320 to the controller 300 wirelessly WL1, received at the at the controller 300. A third key is transmitted from the second external device, separate from the external device 320, to the controller 300 wirelessly WL4. Encrypted data transmitted from the external device 320 to the controller 300 is then decrypted using a derived combined key from the key, the second key and the third key. The external device may be a wearable external device.

The external device 320 may be a handset. The second external device 330 may be a handset.

The second external device 330 may be a server. The second external device 330 may be cloud based.

In some embodiments, the electrical connection C1 between the external device 320 and the controller 300 is achieved by placing a conductive member 201, configured to be in connection with the external device 200, in electrical connection with a skin of the patient for conductive communication C1 with the medical device. In these cases, the medical device and/or external device(s) comprises the necessary features and functionality (described in the respective sections of this document) for performing such conductive communication. The communication may thus be provided with an extra layer of security in addition to the encryption by being electrically confined to the conducting path e.g. external device 320, conductive member 201, conductive connection C1, controller 300, meaning the communication will be excessively difficult to be intercepted by a third party not in physical contact with, or at least proximal to, the patient.

The keys described in this section may in some embodiments be generated based on data sensed by sensors described herein, e.g. using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

Increased security for communication between an external device(s) and an implanted medical device is provided, now described with reference to FIGS. 65a-65c.

In these embodiments, a method for communication between an external device 320 and the implantable controller 300 is provided. The wireless transceiver 308 (included in the controller 300) may in some embodiments comprise sub-transceivers for receiving data from the external device 320 and other external devices 330, e.g. using different frequency bands, modulation schemes etc.

A first step of the method comprises receiving, at the implanted medical device, by a wireless transmission WL1 or otherwise, a first key from an external device 320. The method further comprises receiving, at the implanted medical device, by a wireless transmission WL1, WL2, WL3, a second key.

The second key may be generated by a second external device 330, separate from the external device 320 or by another external device being a generator of the second key on behalf of the second external device 330. The second key may be received at the implanted medical device from anyone of, the external device 320, the second external device 330, and a generator of the second key. The second external device 330 may be controlled by a caretaker, or any other stakeholder. Said another external device may be controlled by a manufacturer of the medical device, or medical staff, caretaker, etc.

In case the medical device is receiving the second key from the external device 320, this means that the second key is routed through the external device from the second external device 330 or from the another external device (generator). In these cases, the medical device and/or external device(s) comprises the necessary features and functionality (described in the respective sections of this document) for performing such routing. Using the external device 320 as a relay, with or without verification from the patient, may provide an extra layer of security as the external device 320 may not need to store or otherwise handle decrypted information. As such, the external device 320 may be lost without losing decrypted information.

The controller 300 comprises a computing unit 306 configured for deriving a combined key by combining the first key and the second key with a third key held by the controller 300, for example in memory 307 of the controller. The combined key may be used for decrypting, by the computing unit 306, encrypted data transmitted by a wireless transmission WL1 from the external device 320 to the controller 300. Optionally, the decrypted data may be used for altering, by the computing unit 306 an operation of the implantable medical device 10. The altering an operation of the implantable medical device may comprise controlling or switching an active unit 302 of the medical device. In some embodiments, the method further comprises at least one of the steps of, based on the decrypted data, updating a control program running in the implant, and operating the implantable medical device 10 using operation instructions in the decrypted data.

In some embodiments, further keys are necessary to derive a combined key for decrypting the encrypted data received at the controller 300. In these embodiments, the first and second key are received as described above. Further, the method comprises receiving, at the implanted medical device, a fourth key from a third external device, the third external device being separate from the external device, deriving a combined key by combining the first, second and fourth key with the third key held by the controller 300, and decrypting the encrypted data, in the controller 300, using the combined key. Optionally, the decrypted data may be used for altering, by the computing unit 306, an operation of the implanted medical device as described above. In some embodiments, the fourth key is routed through the external device from the third external device.

In some embodiments, further security measures are needed before using the decrypted data for altering, by the computing unit 306, an operation of the implantable medical device. For example, an electrical connection C1 between the implantable medical device and the external device 320, using the body as a conductor, may be used for further verification of validity of the decrypted data. The electrical connection C1 may be achieved by placing a conductive member 201, configured to be in connection with the external device, in electrical connection with a skin of the patient for conductive communication C1 with the implantable medical device. The communication may thus be provided with an extra layer of security in addition to the encryption by being electrically confined to the conducting path e.g. external device 320, conductive member 201, conductive connection C1, controller 300, meaning the communication will be excessively difficult to be intercepted by a third party not in physical contact with, or at least proximal to, the patient.

Accordingly, in some embodiments, the method comprising confirming the electrical connection between the controller 300 and the external device 320, and as a result of the confirmation, altering an operation of the implantable medical device based on the decrypted data. The confirmation and authentication of the electrical connection may be performed as described herein under the general features section. In these cases, the implantable medical device and/or external device(s) 320 comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

In some embodiments, the confirmation of the electrical connection comprises: measuring a parameter of the patient, by e.g. a sensor of the implantable medical device 10, measuring the parameter of the patient, by the external device 320, comparing the parameter measured by the implantable medical device to the parameter measured by the external device 320, and authenticating the connection based on the comparison. As mentioned above, as a result of the confirmation, an operation of the implantable medical device may be altered based on the decrypted data.

Further methods for encrypted communication between an external device 320 and an implantable medical device 10 are provided. These methods comprise:

receiving, at the external device 320 by a wireless transceiver 328, a first key, the first key being generated by a second external device 330, separate from the external device 320 or by another external device being a generator of the second key on behalf of the second external device 320, the first key being received from anyone of the second external device 330 and the generator of the second key, receiving, at the external device 320 by the wireless transceiver 328, a second key from the controller 300,

deriving a combined key, by a computing unit 326 of the external device 320, by combining the first key and the second key with a third key held by the external device 320 (e.g. in memory 327),

transmitting encrypted data from the implant to the external device and receiving the encrypted data at the external device by the wireless transceiver 328, and

decrypting, by the computing unit 326, the encrypted data, in the external device 320, using the combined key.

As described above, further keys may be necessary to decrypt the data. Consequently, the wireless transceiver 328 is configured for:

receiving a fourth key from a third external device,

wherein the computing unit 326 is configured for:

deriving a combined key by combining the first, second and fourth key with the third key held by the external device, and

decrypting the encrypted data using the combined key.

In some embodiments, the communication between the controller 300 and the external device 320 needs to be confirmed (authenticated) before decrypting the data. In these cases, the implantable medical device and/or external device(s) comprises the necessary features and functionality (described in the respective sections of this document) for performing such authentication.

These embodiments further increase the security in the communication. In these embodiments the computing unit 326 is configured to confirm the communication between the implantable medical device and the external device, wherein the confirmation comprises:

measuring a parameter of the patient, by the external device 320,

receiving a measured parameter of the patient, from the implantable medical device 10,

comparing the parameter measured by the implantable medical device 320 to the parameter measured by the external device 320,

performing confirmation of the connection based on the comparison, and

as a result of the confirmation, decrypting the encrypted data, in the external device, using the combined key.

One or more of the first, second and third key may comprise a biometric key.

The keys described in this section may in some embodiments be generated based on data sensed by sensors, e.g. using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

Further, increased security for communication between an external device(s) 320, 330 and an implantable medical device is provided, described with reference to FIGS. 65a-65c. The system being configured for enabling communication between an external device 320 and the controller 300 implanted in a patient. The system comprises a conductive member 321 configured to be in connection (electrical/conductive or wireless or otherwise) with the external device, the conductive member 321 being configured to be placed in electrical connection with a skin of the patient for conductive communication C1 with the implantable medical device 10. By using a conductive member 321 as defined herein, an increased security for communication between the external device and the implantable medical device may be achieved. For example, when a sensitive update of a control program of the controller 300 is to be made, or if sensitive data regarding physical parameters of the patient is to be sent to the external device 320 (or otherwise), the conductive member 321 may ensure that the patient is aware of such communication and actively participate in validating that the communication may take place. The conductive member may, by being placed in connection with the skin of the patient, open the conductive communication channel C1 between the external device and the controller to be used for data transmission.

Electrical or conductive communication, such as this or as described under the other embodiments, may be very hard to detect remotely, or at least relatively so, in relation to wireless communications such as radio transmissions. Direct electrical communication may further safeguard the connection between the implantable medical device 10 and the external device 320 from electromagnetic jamming i.e. high-power transmissions other a broad range of radio frequencies aimed at drowning other communications within the frequency range. Electrical or conductive communication will be excessively difficult to be intercepted by a third party not in physical contact with, or at least proximal to, the patient, providing an extra level of security to the communication.

In some embodiments, the conductive member comprises a conductive interface for connecting the conductive member to the external device.

In some embodiments, the conductive member 201 is a device which is plugged into the external device 200, and easily visible and identifiable for simplified usage by the patient. In other embodiments, the conductive member 321 is to a higher degree integrated with the external device 320, for example in the form of a case of the external device 320 comprising a capacitive area configured to be in electrical connection with a skin of the patient. In one example, the case is a mobile phone case (smartphone case) for a mobile phone, but the case may in other embodiments be a case for a personal computer, or a body worn camera or any other suitable type of external device as described herein. The case may for example be connected to the phone using a wire from the case and connected to the headphone port or charging port of the mobile phone.

The conductive communication C1 may be used both for communication between the controller 300 and the external device 320 in any or both directions. Consequently, according to some embodiments, the external device 320 is configured to transmit a conductive communication (conductive data) to the controller 300 via the conductive member 321.

According to some embodiments, the controller 300 is configured to transmit a conductive communication to the external device 320. These embodiments start by placing the conductive member 321, configured to be in connection with the external device 320, in electrical connection with a skin of the patient for conductive communication C1 with the controller 300. The conductive communication between the external device 320 and the controller 300 may follow an electrically/conductively confined path comprising e.g. the external device 320, conductive member 321, conductive connection C1, controller 300.

For the embodiments when the external device 320 transmits data to the controller, the communication may comprise transmitting a conductive communication to the controller 300 by the external device 320.

The transmitted data may comprise instructions for operating the implantable medical device 10. Consequently, some embodiments comprise operating the implantable medical device 10 using operation instructions, by an internal computing unit 306 of the controller 300, wherein the conductive communication C1 comprises instructions for operating the implantable medical device 10. The operation instruction may for example involve adjusting or setting up (e.g. properties or functionality of) the active unit 302 of the implantable medical device 10.

The transmitted data may comprise instructions for updating a control program 310 stored in memory 307 of the controller 300. Consequently, some embodiments comprise updating the control program 310 running in the controller 300, by the internal computing unit 306 of the implantable medical device, wherein the conductive communication comprises instructions for updating the control program 310.

For the embodiments when the controller 300 transmits data to the external device 320, the communication may comprise transmitting conductive communication C1 to the external device 320 by the controller 300. The conductive communication may comprise feedback parameters. Feedback parameters could include battery status, energy level at the controller, the fluid level of the hydraulic restriction device, number of operations that the restriction device has performed, properties, version number etc. relating to functionality of the implantable medical device 10. In other embodiments, the conductive communication C1 comprises data pertaining to least one physiological parameter of the patient, such as blood pressure etc. The physiological parameter(s) may be stored in memory 307 of the controller 300 or sensed in prior (in real time or with delay) to transmitting the conductive communication C1. Consequently, in some embodiments, the implantable medical device 10 comprises a sensor 150 for sensing at least one physiological parameter of the patient, wherein the conductive communication comprises said at least one physiological parameter of the patient.

To further increase security of the communication between the controller 300 and the external device 320, different types of authentication, verification and/or encryption may be employed. In some embodiments, the external device 320 comprises a verification unit 340. The verification unit 340 may be any type of unit suitable for verification of a user, i.e. configured to receive authentication input from a user, for authenticating the conductive communication between the implantable medical device and the external device. In some embodiments, the verification unit and the external device comprises means for collecting authentication input from the user (which may or may not be the patient). Such means may comprise a fingerprint reader, a retina scanner, a camera, a GUI for inputting a code, a microphone, device configured to draw blood, etc. The authentication input may thus comprise a code or any be based on a biometric technique selected from the list of: a fingerprint, a palm vein structure, image recognition, face recognition, iris recognition, a retinal scan, a hand geometry, and genome comparison. The means for collecting the authentication input may alternatively be part of the conductive member which comprise any of the above examples of functionality, such as a fingerprint reader or other type of biometric reader.

In some embodiments, the security may thus be increased by receiving an authentication input from a user by the verification unit 340 of the external device 320, and authenticating the conductive communication between the controller 300 and the external device using the authentication input. Upon a positive authentication, the conductive communication channel C1 may be employed for comprising transmitting a conductive communication to the controller 300 by external device 320 and/or transmitting a conductive communication to the external device 320 by the controller 300. In other embodiments, a positive authentication is needed prior to operating the implantable medical device 10 based on received conductive communication, and/or updating a control program running in the controller 300 as described above.

FIGS. 65a-65c further shows an implantable medical device 10 implanted in a patient and being connected to a sensation generator 381.

The sensation generator 381 may be configured to generate a sensation. The sensation generator 381 may be contained within the implantable medical device 10 or be a separate unit. The sensation generator 381 may be implanted. The sensation generator 381 may also be located so that it is not implanted as such but still is in connection with a patient so that only the patient may experience sensations generated. The controller 300 is configured for storing authentication data, related to the sensation generated by the sensation generator 381.

The controller 300 is further configured for receiving input authentication data from the external device 320. Authentication data related to the sensation generated may by stored by a memory 307 of the controller 300. The authentication data may include information about the generated sensation such that it may be analyzed, e.g. compared, to input authentication data to authenticate the connection, communication or device. Input authentication data relates to information generated by a patient input to the external device 320. The input authentication data may be the actual patient input or an encoded version of the patient input, encoded by the external device 320. Authentication data and input authentication data may comprise a number of sensations or sensation components.

The authentication data may comprise a timestamp. The input authentication data may comprise a timestamp of the input from the patient. The timestamps may be a time of the event such as the generation of a sensation by the sensation generator 381 or the creation of input authentication data by the patient. The timestamps may be encoded. The timestamps may feature arbitrary time units, i.e. not the actual time. Timestamps may be provided by an internal clock 360 of the controller 300 and an external clock 362 of the external device 320. The clocks 360, 362 may be synchronized with each other.

The clocks 360, 362 may be synchronized by using a conductive connection C1 or a wireless connection WL1 for communicating synchronization data from the external device 320, and its respective clock 362, to the controller 300, and its respective clock 360, and vice versa. Synchronization of the clocks 360, 362 may be performed continuously and may not be reliant on secure communication.

Authentication of the connection may comprise calculating a time difference between the timestamp of the sensation and the timestamp of the input from the patient, and upon determining that the time difference is less than a threshold, authenticating the connection. An example of a threshold may be Is. The analysis may also comprise a low threshold as to filter away input from the patient that is faster than normal human response times. The low threshold may e.g. be 50 ms.

Authentication data may comprise a number of times that the sensation is generated by the sensation generator, and wherein the input authentication data comprises an input from the patient relating to a number of times the patient detected the sensation. Authenticating the connection may then comprise: upon determining that the number of times that the authentication data and the input authentication data are equal, authenticating the connection.

A method of authenticating the connection between an implantable medical device 10 implanted in a patient, and an external device 320 according includes the following steps.

Generating, by a sensation generator 381, a sensation detectable by a sense of the patient. The sensation may comprise a plurality of sensation components. The sensation or sensation components may comprise a vibration (e.g. a fixed frequency mechanical vibration), a sound (e.g. a superposition of fixed frequency mechanical vibrations), a photonic signal (e.g. a non-visible light pulse such as an infra-red pulse), a light signal (e.g. a visual light pulse), an electric signal (e.g. an electrical current pulse) or a heat signal (e.g. a thermal pulse). The sensation generator may be implanted, configured to be worn in contact with the skin of the patient or capable of creating sensation without being in physical contact with the patient, such as a beeping alarm.

Sensations may be configured to be consistently felt by a sense of the patient while not risking harm to or affecting internal biological processes of the patient.

The sensation generator 381, may be contained within the controller 300 or be a separate entity connected to the controller 300. The sensation may be generated by a motor (denoted as M in several embodiments shown herein) of the implantable medical device 10, wherein the motor being the sensation generator 381. The sensation may be a vibration, or a sound created by running the motor. The sensation generator 381 may be located close to a skin of the patient and thus also the sensory receptors of the skin. Thereby the strength of some signal types may be reduced.

Storing, by the controller 300, authentication data, related to the generated sensation.

Providing, by the patient input to the external device, resulting in input authentication data.

Providing the input may e.g. comprise an engaging an electrical switch, using a biometric input sensor or entry into digital interface running on the external device 320 to name just a few examples.

Transmitting the input authentication data from the external device to the controller 300. If the step was performed, the analysis may be performed by the controller 300.

Transmitting the authentication data from the implantable medical device 10 to the external device 320. If the step was performed, the analysis may be performed by the external device 320. The wireless connection WL1 or the conductive connection C1 may be used to transmit the authentication data or the input authentication data.

Authenticating the connection based on an analysis of the input authentication data and the authentication data e.g. by comparing a number of sensations generated and experienced or comparing timestamps of the authentication data and the input authentication data. If step was performed, the analysis may be performed by the implantable medical device 10.

Communicating further data between the controller 300 and the external device 320 following positive authentication. The wireless connection WL1 or the conductive connection C1 may be used to communicate the further data. The further data may comprise data for updating a control program 310 running in the controller 300 or operation instructions for operating the implantable medical device 10.

The further data may also comprise data sensed by a sensor 150 connected to the controller 300. The controller may comprise at least one unit having a sleep mode and an active mode, and the unit consumes less energy in the sleep mode than in the active mode. The unit is configured to switch from the sleep mode to the active mode on the basis of at least one signal from the sensor. The unit could for example be a DSP (Digital Signal Processor), another type of processor or a wake-up circuit of the controller, which in turn activates the functions of the controller. The unit may be configured to switch from the sleep mode to the active mode on the basis of a signal from the sensor related to the patient swallowing a number of times and/or on the basis of a signal from the sensor related to the patient swallowing a number of times during a time period. The number of times the patient swallows and the time could be counted/measured and compared with a pre-set or moving threshold value. The controller could further comprise at least one filtering unit configured to filter signals related to at least one of: speech, the swallowing of saliva and chewing. The filter could be a digital filter implemented as hardware or software in the controller and could have the filter characteristics of a high, low or bandpass filter.

If the analysis was performed by the controller 300, the external device 320 may continuously request or receive, information of an authentication status of the connection between the controller 300 and the external device 320, and upon determining, at the external device 320, that the connection is authenticated, transmitting further data from the external device 320 to the controller 300.

If the analysis was performed by the external device 320, the controller 300 may continuously request or receive, information of an authentication status of the connection between the controller 300 and the external device 320, and upon determining, at the controller 300, that the connection is authenticated, transmitting further data from the controller 300 to the external device 320.

A main advantage of authenticating a connection according to this method is that only the patient may be able to experience the sensation. Thus, only the patient may be able to authenticate the connection by providing authentication input corresponding to the sensation generation.

The sensation generator 381, sensation, sensation components, authentication data, input authentication data, and further data may be further described herein. In these cases, the implantable medical device 10 and/or external device(s) comprises the necessary features and functionality (described in the respective sections of this document). Further information and definitions can be found in this document in conjunction with the other aspects.

The method may further comprise transmitting further data between the controller 300 and the external device, wherein the further data is used or acted upon, only after authentication of the connection is performed.

The analysis or step of analyzing may be understood as a comparison or a step of comparing.

In one method, increased security for communication between an external device(s) and an implanted controller is provided. FIGS. 65a-65c show an implantable medical device 10 comprising a controller 300 and an external device 320 which may form a system.

The controller 300 comprises a transceiver 308, 303 configured to establish a connection with an external device 320, i.e. with a corresponding transceiver 328, 323. The connection may be an electrical connection C1 using the transceivers 303, 323, or a wireless connection WL1 using the transceivers 308, 328. The controller 300 further comprises a computing unit 306 configured to verify the authenticity of instructions received at the transceiver 308, 303 from the external device 320. In this aspect, the concept of using previously transmitted instructions for verifying a currently transmitted instructions are employed. Consequently, the transmitting node (in this case the external device) need to be aware of previously instructions transmitted to the implantable medical device, which reduces the risk of a malicious device instructing the implant without having the authority to do so.

In an embodiment, the computing unit 306 is configured to verify the authenticity of instructions received at the transceiver 308, 303 by extracting a previously transmitted set of instructions from a first combined set of instructions received by the transceiver. The external device 320 may thus comprise an external device comprising a computing unit 326 configured for: combining a first set of instructions with a previously transmitted set of instructions, forming a combined set of instructions, and transmitting the combined set of instructions to the implantable medical device. The previously transmitted set of instructions, or a representation thereof, may be stored in memory 327 of the external device 320.

The combined set of instructions may have a data format which facilitates such extraction, for example including metadata identifying data relating to the previously transmitted set of instructions in the combined set of instructions. In some embodiments, the combined set of instructions comprises the first set of instructions and a cryptographic hash of the previously transmitted set of instructions. Consequently, the method comprises combining, at the external device, a first set of instructions with a previously transmitted set of instructions, forming a first combined set of instructions. A cryptographic hash function is a special class of hash function that has certain properties which make it suitable for use in cryptography. It is a mathematical algorithm that maps data of arbitrary size to a bit string of a fixed size (a hash) and is designed to be a one-way function, that is, a function which is infeasible to invert. Examples include MD5, SHA1, SHA 256, etc. Increased security is thus achieved.

The first combined set of instructions is then transmitted to the implanted controller 300, where it is received by e.g. the transceiver 303, 308. The first combined set of instructions may be transmitted to the implantable medical device using a proprietary network protocol. The first combined set of instructions may be transmitted to the controller 300 using a standard network protocol. In these cases, the controller 300 and/or external device(s) comprises the necessary features and functionality (described in the respective sections of this document) for performing transmission of data. By using different communication protocols, at the external device 320, for communication with the controller 300 and with a second external device 330, an extra layer of security is added as the communication between controller 300 and the external device 320 may be made less directly accessible to remote third parties.

At the controller 300, the computing unit 306 verifies the authenticity of the received first combined set of instructions, by: extracting the previously transmitted set of instructions from the first combined set of instructions, and comparing the extracted previously transmitted set of instructions with previously received instructions stored in the implantable medical device.

Upon determining that the extracted previously transmitted set of instructions equals the previously received instructions stored in the controller 300, the authenticity of the received first combined set of instructions may be determined as valid, and consequently, the first set of instructions may be safely run at the controller 300, and the first combined set of instructions may be stored in memory 307 of the controller 300, to be used for verifying a subsequent received set of instructions.

In some embodiments, upon determining by the internal computing unit 306 that the extracted previously transmitted set of instructions differs from the previously received instructions stored in the controller 300, feedback related to an unauthorized attempt to instruct the implantable medical device 10 may be provided. For example, the transceiver 308, 303 may send out a distress signal to e.g. the external device 320 or to any other connected devices. The controller 300 may otherwise inform the patient that something is wrong by e.g. vibration or audio. The implantable medical device 10 may be run in safe mode, using a preconfigured control program which is stored in memory 307 of the controller 300 and specifically set up for these situations, e.g. by requiring specific encoding to instruct the implantable medical device 10, or only allow a predetermined device (e.g. provided by the manufacturer) to instruct the implantable medical device 10. In some embodiments, when receiving such feedback at the external device 320, the external device 320 retransmits the first combined set of instructions again, since the unauthorized attempt may in reality be an error in transmission (where bits of the combined set of instructions are lost in transmission), and where the attempt to instruct the implantable medical device 10 is indeed authorized.

The step of comparing the extracted previously transmitted set of instructions with previously received instructions stored in the controller 300 may be done in different ways. For example, the step of comparing the extracted previously transmitted set of instructions with previously received instructions stored in the controller 300 comprises calculating a difference between the extracted previously transmitted set of instructions with previously received instructions stored in the controller 300, and comparing the difference with a threshold value, wherein the extracted previously transmitted set of instructions is determined to equal the previously received instructions stored in the controller 300 in the case of the difference value not exceeding the threshold value. This embodiment may be used when received instructions is stored in clear text, or a representation thereof, in the controller 300, and where the combined set of instructions, transmitted from the external device also includes such a representation of the previously transmitted instructions. This embodiment may be robust against error in transmission where bits of information are lost or otherwise scrambled.

In other embodiments, the combined set of instructions comprises the first set of instructions and a cryptographic hash of the previously transmitted set of instructions, wherein the method further comprises, at the controller 300, calculating a cryptographic hash of the previously received instructions stored in the controller 300 and comparing the calculated cryptographic hash to the cryptographic hash included in the first combined set of instructions. This embodiment provides increased security since the cryptographic hash is difficult to decode or forge.

The above way of verifying the authenticity of received instructions at the controller 300 may be iteratively employed for further sets if instructions.

To further increase security, the transmission of a first set of instructions, to be stored at the controller 300 for verifying subsequent sets of combined instructions, where each set of received combined instructions will comprise data which in some form will represent, or be based on, the first set of instruction, may be performed.

In one example, the external device 320 may be adapted to communicate with the controller 300 using two separate communication methods. A communication range of a first communication method WL1 may be less than a communication range of a second communication method WL2. A method may comprise the steps of: sending a first part of a key from the external device 320 to the controller 300, using the first communication method WL1 and sending a second part of the key from the external device 320 to the controller 300, using the second communication method WL2. The method may further comprise deriving, in the controller 300, a combined key from the first part of the key and the second part of the key and decrypting the encrypted data, in the controller 300, using the combined key. The encrypted data may also be sent from the external device 320 to the controller 300 using the second communication method WL2. The method may then further comprise confirming an electrical connection C1 between the controller 300 and the external device 320 and as a result of the confirmation, decrypting the encrypted data in the controller 300 and using the decrypted data for instructing the controller 300.

The method may also comprise placing a conductive member 321, configured to be in connection with the external device 320, in electrical connection with a skin of the patient for conductive communication with the controller 300. By means of the electrical connection an extra layer of security is added as a potential hacker would have to be in contact with the patient to access or affect the operation of the implantable medical device 10.

Using a plurality of communication methods, may increase the security of the authentication and the communication with the implantable medical device 10 as more than one channel for communication may need to be hacked or hijacked by an unauthorized entity to gain access to the implantable medical device 10 or the communication.

The electrical connection C1 the conductive member 321 and conductive communication may be further described herein in the general definitions section. In these cases, the controller 300 and/or external device 320 comprise the necessary features and functionality (described in the respective sections of this document).

It should also be noted that any one of the first and second communication methods WL1, WL2 may be needed to be confirmed in order to decrypt the encrypted data in the controller 300 and using the decrypted data for instructing the implantable medical device 10.

The method may further comprise the step of wirelessly receiving, at the controller 300, a third part of the key from the second external device 330. In this case, the combined key may be derived from the first part of the key, the second part of the key and the third part of the key.

The first communication method WL1 may be a wireless form of communication. The first communication method WL1 may preferably be a form of electromagnetic or radio-based communication however, other forms of communication are not excluded. The first communication method WL1 may comprise or be related to the items of the following list: Radio-frequency identification (RFID), Bluetooth, Bluetooth 5, Bluetooth Low Energy (BLE), Near Field Communication (NFC), NFC-V, Infrared (IR) based communication, Ultrasound based communication.

RFID communication may enable the use of a passive receiver circuit such as those in a RFID access/key or payment card. IR based communication may comprise fiber optical communication and IR diodes. IR diodes may alternatively be used directly, without a fiber, such as in television remote control devices. Ultrasound based communication may be based on the non-invasive, ultrasound imaging found in use for medical purposes such as monitoring the development of mammal fetuses.

The first communication method WL1 may use a specific frequency band. The frequency band of the first communication method WL1 may have a center frequency of 13.56 MHz or 27.12 MHz.

These bands may be referred to as industrial, scientific and medical (ISM) radio bands. Other ISM bands not mentioned here may also be utilized for the communication methods WL1, WL2. A bandwidth of the 13.56 MHz centered band may be 14 kHz and a bandwidth of the 27.12 MHz centered band may be 326 kHz.

The communication range of the first communication method WL1 may be less than 10 meters, preferably less than 2 meters, more preferably less than 1 meter and most preferably less than 20 centimeters. The communication range of the first communication method WL1 may be limited by adjusting a frequency and/or a phase of the communication. Different frequencies may have different rates of attenuation. By implementing a short communication range of the first communication method, security may be increased since it may be ensured or made probable that the external device is under control of the patient (holding the external device close to the implant)

The communication range of the first communication method WL1 should be evaluated by assuming that a patient's body, tissue, and bones present the propagation medium. Such a propagation medium may present different attenuation rates as compared to a free space of an air-filled atmosphere or a vacuum.

By restricting the communication range, it may be established that the external device communicating with the implanted controller 300 is in fact on, or at least proximal to, the patient. This may add extra security to the communication.

The second communication method WL2 may be a wireless form of communication. The second communication method WL2 may preferably be a form of electromagnetic or radio-based communication. The second communication method WL2 may be based on telecommunication methods.

The second communication method WL2 may comprise or be related to the items of the following list: Wireless Local Area Network (WLAN), Bluetooth, Bluetooth 5, BLE, GSM or 2G (2nd generation cellular technology), 3G, 4G, 5G.

The second communication method WL2 may utilize the ISM bands as mentioned in the above for the first communication method WL1.

A communication range of the second communication method WL2 may be longer than the communication range of the first communication method WL1. The communication range of the second communication method WL2 may preferably be longer than 10 meters, more preferably longer than 50 meters, and most preferably longer than 100 meters.

Encrypted data may comprise instructions for updating a control program 310 running in the implantable medical device 10. Encrypted data may further comprise instructions for operating the implantable medical device 10.

In one embodiment, the implantable medical device 10 may transmit data to an external device 320 which may add an additional layer of encryption and transmit the data to a second external device 330, described with reference to FIGS. 65a-65c. By having the external device add an additional layer of encryption, less computing resources may be needed in the implanted controller 300, as the controller 300 may transmit unencrypted data or data encrypted using a less secure or less computing resource requiring encryption. In this way, data can still be relatively securely transmitted to a third device. The transmission of data can be performed using any of the method described herein in addition to the method or in the system described below.

Thus, in an embodiment, a system is provided. The system comprises an implantable medical device 10 comprising a controller 300 configured to transmit data from the body of the patient to an external device 320, and an encryption unit 382 for encrypting the data to be transmitted. The system further comprises an external device 320 configured to receive the data transmitted by the controller 300, encrypt the received data using a first key and transmit the encrypted received data to a third external device 330. The encryption can be performed using any of the keys described above or below. In some embodiments, the external device 320 is configured to decrypt the data received from the controller 300 before encrypting and transmitting the data. Alternatively, the external device 320 may encrypt and transmit the data received from the controller 300 without decrypting it first.

In one example, the encryption unit 382 is configured to encrypt the data to be transmitted using a second key. The first key or the second key may, for example, information specific to the implantable medical device 10, a secret key associated with the external device 320, an identifier of the implantable medical device 10 or an identifier of the controller 300. The second key could be a key transmitted by the external device 320 to the controller 300. In some examples, the second key is a combined key comprising a third key received by the controller 300 from the external device 320.

The first key may be a combined key comprising a fourth key, wherein the fourth key is received by the external device 320 from a fourth device. The fourth device may be a verification unit, either comprised in the external device, or external to the external device and connected to it. The verification unit may have a sensor 350 for verification, such as a fingerprint sensor. More details in regard to this will be described below. Alternatively, the verification unit may be a generator, as described above.

The system may be configured to perform a method for transmitting data using a sensed parameter. The method may comprise transmitting a parameter measured by the external device 320 from the external device 320 to the controller 300. In this case, the comparison of the parameter of the patient measured by the external device 320 and the parameter of the patient measured by the controller 300 may be performed by the controller 300. The implantable medical device 10 may comprise a first sensor 150 for measuring the parameter of the patient at the implantable medical device 10. The external device 320 may comprise an external sensor 350 for measuring the parameter of the patient at the external device 320.

Authentication of the connection between the controller 300 and the external device 320 may be performed automatically without input, authentication, or verification from a user or patient. This is because the comparison of parameters measured internally and externally, by the internal and external sensors 351, 350 respectively may be enough to authenticate the connection. This may typically be the case when the parameter of the patient is related to an automatically occurring physiological function of the patient such as e.g. a pulse of the patient. Certain types of authentication may however require actions from the patient, e.g. having the patient perform specific movements.

In the embodiments described herein, the controller 300 may comprise or be connected to a sensation generator 381 as described above. In response to an event in the implantable medical device, such as a reset, a restart, receipt of new instructions, receipt of a new configuration or update, installation or activation of new instructions or configuration or update, the controller 300 may be configured to cause the sensation generator 381 to generate a sensation detectable by the patient in which the implantable medical device 10 is implanted. In some examples, the user may after the sensation verify an action, for example via a user interface of an external device 320.

The implantable medical device 10 may further implement a method for improving the security of the data transmitted from the controller 300. The method, for encrypted communication between a controller 300, when implanted in a patient's body, and an external device 320, comprises encoding or encrypting, by the controller 300 or a processor 306 comprised in or connected to the controller 300, data relating to the implantable medical device 10 or the operation thereof; transmitting, by the controller 300, the data; receiving, by a second communication unit comprised the external device 320, the data; encrypting, by the external device 320, the data using an encryption key to obtain encrypted data; and transmitting the encrypted data to a third external device 330. In this way, the external device 320 may add or exchange the encryption, or add an extra layer of encryption, to the data transmitted by the controller 300. When the controller 300 encodes the data to be transmitted it may be configured to not encrypt the data before transmitting, or only using a light-weight encryption, thus not needing as much processing power as if the controller were to fully encrypt the data before the transmission.

The encrypting, by the controller 300, may comprise encrypting the data using a second key.

The encryption using the second key may be a more light-weight encryption than the encryption performed by the external device using the second key, i.e. an encryption that does not require as much computing resources as the encryption performed by the external device 320.

The first or the second key may comprise a private key exchanged as described above with reference to encryption and authentication, or the first or the second key may comprise an information specific to the implantable medical device 10, a secret key associated with the external device, an identifier of the implantable medical device 10 or an identifier of the controller 300. They may be combined keys as described in this description, and the content of the keys, any combination of keys, and the exchange of a key or keys is described in the encryption and/or authentication section.

In an embodiment, the implantable medical device 10 comprises at least one sensor for sensing at least one physiological parameter of the patient or a functional parameter of the implantable medical device 10, now described with reference to FIGS. 65a-65c. The sensor 351 may, for example, be a pressure sensor, an electrical sensor, a clock, a temperature sensor, a motion sensor, an optical sensor, a acoustic sensor, an ultrasonic sensor. The sensor 351 is configured to periodically sense the parameter and the controller 300 is configured to, in response to the sensed parameter being above a predetermined threshold, wirelessly broadcast information relating to the sensed parameter. The controller 300 may be configured to broadcast the information using a short to mid-range transmitting protocol, such as a Radio Frequency type protocol, a RFID type protocol, a WLAN type protocol, a Bluetooth type protocol, a BLE type protocol, a NFC type protocol, a 3G/4G/5G type protocol, or a GSM type protocol.

The controller of the implant may be connected to the sensor 351 and be configured to anonymize the information before it is transmitted. The transmission of data may also be called broadcasting of data.

In addition to or as an alternative to transmitting the data when the sensed parameter is above a predetermined threshold, the controller 300 may be configured to broadcast the information periodically. The controller 300 may be configured to broadcast the information in response to a second parameter being above a predetermined threshold. The second parameter may, for example, be related to the controller 300 itself, such as a free memory or free storage space parameter, or a battery status parameter. When the implantable medical device 10 comprises an implantable energy storage unit and an energy storage unit indicator, the energy storage unit indicator is configured to indicate a functional status of the implantable energy storage unit and the indication may be comprised in the transmitted data.

The functional status may indicate at least one of charge level and temperature of the implantable energy storage unit.

In some embodiments the external device 320 is configured to receive the broadcasted information, encrypt the received information using an encryption key and transmit the encrypted received information. In this way, the external device 320 may add an additional layer of encryption or exchange the encryption performed by the controller 300.

In an embodiment, the controller 300 is configured to transmit the data using the body of the patient as a conductor C1, and the external device 320 is configured to receive the data via the body. Alternatively, or in combination, the controller 300 of the implant is configured to transmit the data wirelessly to the external device WL2.

Thus, the controller 300 may implement a method for transmitting data from the controller 300 comprising a processor 306, comprising: obtaining sensor measurement data via a sensor 150 connected to or comprised in the controller 300, the sensor measurement relating to at least one physiological parameter of the patient or a functional parameter of the implantable medical device 10, and transmitting by the controller 300 the sensor measurement data in response to the sensor measurement being above a predetermined threshold, wherein the sensor 150 is configured to periodically sense the parameter. The method may further comprise broadcasting the sensor measurement data, to be received by an external device 320. The transmitting or broadcasting may comprise using at least one of a Radio Frequency type protocol, RFID type protocol, WLAN type protocol, Bluetooth type protocol, BLE type protocol, NFC type protocol, 3G/4G/5G type protocol, or a GSM type protocol.

The method may further comprise, at the processor 306, anonymizing, by the processor, the sensor measurement data before it is transmitted, or encrypting the sensor measurement data, using an encryptor 382 comprised in the processing unit 306, before it is transmitted. The transmitting of the data may further comprise to encode the data before the transmitting. The type of encoding may be dependent on the communication channel or the protocol used for the transmission.

The transmitting may be performed periodically, or in response to a signal received by the processor, for example, by an internal part of the implantable medical device 10 such as a sensor 150, or by an external device 320.

The parameter may, for example, be at least one of a functional parameter of the implantable medical device 10 (such as a battery parameter, a free memory parameter, a temperature, a pressure, an error count, a status of any of the control programs, or any other functional parameter mentioned in this description) or a parameter relating to the patient (such as a temperature, a blood pressure, or any other parameter mentioned in this description). In one example, the implantable medical device 10 comprises an implantable energy storage unit 40 and an energy storage unit indicator 304c, and the energy storage unit indicator 304c is configured to indicate a functional status of the implantable energy storage unit 40, and the sensor measurement comprises data related to the energy storage unit indicator.

In one example, the transmitting comprises transmitting the sensor measurement to an internal processor 306 configured to cause a sensation generator 381 to cause a sensation detectable by the patient in which the implantable medical device 100 is implanted.

The method may be implemented in a system comprising the implantable medical device 100 and an external device 320, and further comprise receiving the sensor measurement data at the external device 320, and, at the external device 320, encrypting the sensor measurement data using a key to obtain encrypted data, and, transmitting the encrypted data. The transmitting may, for example, be performed wirelessly WL3 or conductively C1.

In the examples or embodiments transmitting data from or to the implantable medical device 10, the following method may be implanted in order to verify the integrity of the data, described with reference to FIGS. 65a-65b. By verifying the integrity of the data, an external device 320 or a processor 306 comprised in the controller 300 may verify that the data has not been corrupted or tampered with during the transmission. In some examples, data integrity for data communicated between a controller 300 and an external device 320 or between an external device 320 and the controller 300 may be performed using a cyclic redundancy check.

Thus, in a first example, a method for evaluating a parameter of a controller 300 implanted in a patient is described. The controller 300 comprises a processor 306 and a sensor 150 for measuring the parameter. The method comprises measuring, using the sensor 150, the functional parameter to obtain measurement data; establishing a connection between the internal controller 300 and an external device 320 configured to receive data from the implant; determining, by the processor 306, a cryptographic hash or a metadata relating to the measurement data and adapted to be used by the external device 320 to verify the integrity of the received data; transmitting the cryptographic hash or metadata; and transmitting, from the controller 300, the measurement data.

The parameter may, for example, be a parameter of the controller 300, such as a temperature, a pressure, a battery status indicator, a time period length, s pressure at a restriction device, a pressure at a sphincter, or a physiological parameter of the patient, such as a pulse, a blood pressure, or a temperature. In some examples, multiple parameters may be used.

The method may further comprise evaluating the measurement data relating to the functional parameter. By evaluating it may be meant to determine if the parameter is exceeding or less than a predetermined value, to extract another parameter from the measurement data, compare the another parameter to a predetermined value, or displaying the another parameter to a user. For example, the method may further comprise, at the external device 320, to determining, based on the evaluating, that the implantable medical device 10 is functioning correctly, or determining based on the evaluating that the implantable medical device 10 is not functioning correctly.

If it is determined that the implantable medical device 10 is not functioning correctly, the method may further comprise sending, from the external device 320, a corrective command to the controller 300, receiving the corrective command at the controller 300, and by running the corrective command correcting the functioning of the implantable medical device 10 according to the corrective command.

The method may further comprise, at the external device 320, receiving the transmitted cryptographic hash or metadata, receiving the measurement data, and verifying the integrity of the measurement data using the cryptographic hash or metadata. The cryptographic hash algorithm be any type of hash algorithm, i.e. an algorithm comprising a one-way function configured to have an input data of any length as input and produce a fixed-length hash value. For example, the cryptographic hash algorithm may be MD5, SHA1, SHA 256, etc.

In some examples, the cryptographic hash is a signature obtained by using a private key of the controller 300, and wherein the verifying, by the external device 320, comprises verifying the signature using a public key corresponding to the private key.

When using a cryptographic hash, the method may further comprise calculating a second cryptographic hash for the received measurement data using a same cryptographic hash algorithm as the processor, and determining that the measurement data has been correctly received based on that the cryptographic hash and the second cryptographic hash are equal (i.e. have the same value).

When using a metadata the verifying the integrity of the data may comprises obtaining a second metadata for the received measurement data relating to the functional parameter, and determining that the data has been correctly received based on that metadata and the second metadata are equal. The metadata may, for example, be a length of the data or a timestamp. In some examples the measurement data is transmitted in a plurality of data packets. In those examples, the cryptographic hash or metadata comprises a plurality of cryptographic hashes or metadata each corresponding to a respective data packet, and the transmitting of each the cryptographic hashes or metadata is performed for each of the corresponding data packets.

A similar method may be utilized for communicating instructions from an external device 320 to a controller 300 implanted in a patient. The method comprises establishing a first connection between the external device 320 and the controller 300, establishing a second connection between a second external device 330 and the controller 300, transmitting, from the external device 320, a first set of instructions to the controller 300 over the first connection, transmitting, from the second external device 330, a first cryptographic hash or metadata corresponding to the first set of instructions to the controller 300, and, at the controller 300, verifying the integrity of the first set of instructions and the first cryptographic hash or metadata, based on the first cryptographic hash or metadata. The external device 320 may be separate from the second external device 330.

The first connections may be established between the controller 300 and a transceiver of the external communication unit 323. In some examples, the communication using the second connection is performed using a different protocol than a protocol used for communication using the first communication channel. In some examples, the first connection is a wireless connection and the second connection is an electrical connection. The second connection may, for example, be an electrical connection using the patient's body as a conductor (using 321). The protocols and ways of communicating may be any communication protocols described in this description with reference to C1, and WL1-WL4. The establishing of the first and second connections are performed according to the communication protocol used for each of the first and the second connections.

When using a cryptographic hash, the verifying the integrity of the first set of instructions may comprise calculating a second cryptographic hash for the received first set of instructions using a same cryptographic hash algorithm as the processor 306, and determining that the first set of instructions has been correctly received based on that the cryptographic hash and the second cryptographic hash are equal. The cryptographic hash may, for example, be a signature obtained by using a private key of the implantable medical device 10, and wherein the verifying comprises verifying the signature using a public key corresponding to the private key. In some examples, the cryptographic hash is a signature obtained by using a private key of the implantable medical device 10, and wherein the verifying comprises verifying the signature using a public key corresponding to the private key. The private keys and public keys, as well as the exchange or transmittal of keys have been described in this description. Alternatively, other well-known methods can be used for transmitting or exchanging a key or keys between the external device 320 and the controller 300.

When using a metadata, and wherein the verifying the integrity of the data may comprise obtaining a second metadata for the received first set of instructions, and determining that the first set of instructions has been correctly received based on that metadata and the second metadata are equal. The metadata may, for example, be any type of data relating to the data to be transmitted, in this example the first set of instructions. For example, the metadata may be a length of the data to be transmitted, a timestamp on which the data was transmitted or retrieved or obtained, a size, a number of packets, or a packet identifier.

In some examples, the controller 300 may transmit data to an external device 320 relating to the data information in order to verify that the received data is correct. The method may thus further comprise, transmitting, by the controller 300, information relating to the received first set of instructions, receiving, by the external device 320, the information, and verifying, by the external device 320, that the information corresponds to the first set of instructions sent by the external device 320. The information may, for example, comprise a length of the first set of instructions.

The method may further comprise, at the controller 300, verifying the authenticity of the first set of instructions by i. calculating a second cryptographic hash for the first set of instructions, ii. comparing the second cryptographic hash with the first cryptographic hash, iii. determining that the first set of instructions are authentic based on that the second cryptographic hash is equal to the first cryptographic hash, and upon verification of the authenticity of the first set of instructions, storing them at the controller 300.

In some examples, the first set of instructions comprises a cryptographic hash corresponding to a previous set of instruction, as described in other parts of this description.

In some examples, the first set of instructions may comprise a measurement relating to the patient of the body for authentication, as described in other parts of this description.

A system and a method for communication of instructions or control signals between an external device 320 and an implantable medical device 10 will now be described with reference to FIGS. 65a-65c.

The system shown in FIGS. 65a-65c comprises an implantable medical device 10, a first external device 320, and a second external device 330. The implantable medical device a controller 300.

The controller 300 is adapted to receive an instruction from an external device 320 over the communication channel WL1, C1 and run the instruction to control a function of the medical device 10.

The communication channel WL1, C1 may be any type of communication channel, such as a wireless connection WL1 or a conductive connection C1 described herein. For example, the wireless connection may comprise at least one of the following protocols: Radio Frequency type protocol, RFID type protocol, WLAN type protocol, Bluetooth type protocol, a BLE type protocol, a NFC type protocol, a 3G/4G/5G/6G type protocol, a GSM type protocol, and/or Bluetooth 5.

The first external device 320 is adapted to receive, such as through a user interface, or determine an instruction to be transmitted to the implantable medical device 10. The determination of the instruction may, for example, be based on received data from the implantable medical device 10, such as measurement data or data relating to a state of the implantable medical device 10, such as a battery status or a free memory status. The first external device 320 may be any type of device capable of transmitting information to the implantable medical device and capable of determining or receiving an instruction to be transmitted to the implantable medical device 10. In a preferred embodiment, the first external device 320 is a hand-held device, such as a smartphone, smartwatch, tablet etc. handled by the patient, having a user interface for receiving an instruction from a user, such as the patient or a caregiver.

The first external device 320 is further adapted to transmit the instruction to a second external device 330 via communication channel WL3. The second external device 320 is adapted to receive the instruction, encrypt the instruction using an encryption key, and then transmit the encrypted instruction to the implantable medical device 10. The implantable medical device 10 is configured to receive the instruction at the controller 300. The controller 300 thus comprises a wired transceiver or a wireless transceiver for receiving the instruction. The implantable medical device 10 is configured to decrypt the received instruction. The decryption may be performed using a decryption key corresponding to the encryption key. The encryption key, the decryption key and methods for encryption/decryption and exchange of keys may be performed as described in the “general definition of features” or as described with reference to FIGS. 65a-65c. Further, there are many known methods for encrypting data which the skilled person would understand to be usable in this example.

The second external device 330 may be any computing device capable of receiving, encrypting and transmitting data as described above. For example, the second external device 320 may be a network device, such as a network server, or it may be an encryption device communicatively coupled to the first external device.

The instruction may be a single instruction for running a specific function or method in the implantable medical device 10, a value for a parameter of the implantable medical device 10, or a set of sub-steps to be performed by the controller 300 comprised in the implantable medical device 10.

In this way, the instruction for controlling a function of the implantable medical device 10 may be received at the first external device 320 and transmitted to the implantable medical device 10 via the second external device 330. By having a second external device 330 encrypting the instruction before transmitting it to the implantable medical device 10, the instruction may be verified by the second external device 330 and the first external device 320 may function so as to relay the instruction. In some alternatives, the second external device 330 may transmit the instruction directly to the implantable medical device 10. This may provide an increased security as the instruction sent to the implantable medical device 10 may be verified by the second external device 330, which, for example, may be a proprietary device managed by the medical professional responsible for the implantable medical device 10. Further, by having the second external device 330 verifying and encrypting the instruction, the responsibility authenticity and/or correctness of the instruction may lie with the second external device 330, which may be beneficial for regulatory purposes, as the first external device 320 may not be considered as the instructor of the implantable medical device 10.

Further, the second external device 330 may verify that the instruction is correct before encrypting or signing and transmitting it to the implantable medical device 10. The second external device 330 may, for example, verify that the instruction is correct by comparing the instruction with a predetermined set of instructions, and if the instruction is comprised in the predetermined set of instructions determine that the instruction is correct. If the instruction comprises a plurality of sub-steps, the second external device 330 may determine that the instruction is correct if all the sub-steps are comprised in the predetermined set of instructions. If the instruction comprises a value for a parameter of the implantable medical device 10, the second external device 330 may verify that the value is within a predetermined range for the parameter. The second external device 320 may thus comprise a predetermined set of instructions, or a predetermined interval or threshold value for a value of a parameter, stored at an internal or external memory.

The second external device 330 may be configured to reject the instruction, i.e. to not encrypt and transmit the instruction to the implantable medical device 10, if the verification of the instruction would fail. For example, the second external device 330 determines that the instruction or any sub-step of the instruction is not comprised in the predetermined set of instructions, or if a value for a parameter is not within a predetermined interval, the second external device 330 may determine that the verification has failed.

In some embodiments, the implantable medical device 10 may be configured to verify the instruction. The verification of the instruction may be performed in the same way as described with reference to FIGS. 65a-65c. If the verification is performed by comparing the instruction or any sub-steps of the instruction with a predetermined set of instructions, the controller 300 may comprise a predetermined set of instructions. The predetermined set of instructions may, for example, be stored in an internal memory of the controller 300. Similarly, the controller 300 may store predetermined reference intervals for any parameter that can be set, and the controller 300 may be configured to compare a received value for a parameter to such a predetermined reference interval. If the verification of the instruction would fail, the controller 300 may be configured to reject the instruction, i.e. not run the instruction.

In an alternative to encrypting and decrypting the instruction, the instruction may be signed by the second external device 330 using a cryptographic hash, and the controller 300 may be configured to verify that the signature is correct before running the instruction.

A corresponding method for transmitting an instruction will now be described with reference to FIGS. 65a-65c. The instruction may relate to a function of the implantable medical device, such as an instruction to run a function or method of the implantable medical device, or to set a value of a parameter of the implantable medical device. The method comprises: transmitting an instruction for the implantable medical device from the first external device 300 to a second external device 320, the instruction relating to a function of the implantable medical device 10, encrypting, at the second external device 330 using a first encryption key, the instruction into an encrypted instruction, and transmitting the encrypted instruction from the second external device 330 to the implantable medical device 10, decrypting, at the implantable medical device, the instructions using a second encryption key corresponding to the first encryption key. The steps performed by or at the implantable medical device may be executed by the controller 300.

The instruction may be any type of instruction for controlling a function of the implantable medical device. For example, the instruction may be an instruction to run a function or method of the implantable medical device 10 or controller 300, an instruction comprising a plurality of sub-steps to be run at the controller 300, or a value for a parameter at the controller 300. The first external device 320 may, for example, receive the instruction from a user via a user interface displayed at or connected to the first external device 320. In another example, the first external device 320 may determine the instruction in response to data received from the implantable medical device 10, such as measurement data, or from another external device. Thus, in some examples, the method may further comprise receiving, at the first external device 320, an instruction to be transmitted to the implantable medical device 10. The method may further comprise displaying a user interface for receiving the instruction. In another example, the method comprises determining, at the first external device 320, an instruction to be transmitted to the implantable medical device 10.

In some embodiments, the transmitting of the encrypted instruction from the second external device 330 to the implantable medical device 10 comprises transmitting the encrypted instruction from the second external device 330 to the first external device 320, and transmitting the encrypted instruction from the first external device 320 to the controller 300 of the implantable medical device 10. In other words, the first external device 320 may relay the encrypted instruction from the second external device 330 to the controller 300, preferably without decrypting the instruction before transmitting it.

The method may further comprise to, at the controller 300, running the instruction or performing the instruction. The running of the instruction may be performed by an internal computing unit or a processor 306 comprised in the controller 300, and may, for example, cause the internal computing unit or processor 306 to instruct the implantable medical device 302 to perform an action.

The method may further comprise verifying, at the second external device 330, that the instructions are correct. The verifying may be performed as described above with reference to the corresponding system.

The method may further comprise verifying, at the controller 300, that the instructions are correct. The verifying may be performed as described above with reference to the corresponding system.

The method may further comprise authenticating the connection between the first external device 320 and the controller 300 over which the encrypted instruction is to be transmitted. The authentication may be performed as described herein.

As described above, a control program of the controller 300 may be updatable, configurable or replaceable. A system and a method for updating or configuring a control program of the controller 300 is now described with reference to FIGS. 65a-65c. The controller may comprise an internal computing unit 306 configured to control a function of the implantable medical device 10, the internal computing unit 306 comprises an internal memory 307 configured to store: i. a first control program 310 for controlling the internal computing unit, and ii. a second, configurable or updatable, with predefined program steps, control program 312 for controlling said function of the implantable medical device 10, and iii. a set of predefined program steps for updating the second control program 312. The controller 300 is configured to communicate with an external device 320. The internal computing unit 306 is configured to receive an update to the second control program 312 via the controller 300, and a verification function of, connected to, or transmitted to the controller 300. The verification function is configured to verify that the received update to the second control program 312 comprises program steps comprised in the set of predefined program steps. In this way, the updating or programming of the second control program may be performed using predefined program steps, which may decrease the risk that the new or updated control program is incorrect or comprises malicious software, such as a virus, spyware or a malware.

The predefined program steps may comprise setting a variable related to a pressure, a time, a minimum or maximum temperature, a current, a voltage, an intensity, a frequency, an amplitude of electrical stimulation, a feedback mode (sensorics or other), a post-operative mode or a normal mode, a catheter mode, a fibrotic tissue mode (for example semi-open), an time open after urination, a time open after urination before bed-time.

The verification function may be configured to reject the update in response to the update comprising program steps not comprised in the set of predefined program steps and/or be configured to allow the update in response to the update only comprising program steps comprised in the set of predefined program steps.

The internal computing unit 306 may be configured to install the update in response to a positive verification, for example by a user using an external device, by a button or similarly pressed by a user, or by another external signal.

The authentication or verification of communications between the implant and an external device has been described above.

When updating a control program of the controller 300, it may be beneficial to transmit a confirmation to a user or to an external device or system. Such a method is now described with reference to FIGS. 65a-65b.

The method for updating a control program of a controller 300 comprised in the implantable medical device 10 according to any of the embodiments herein. The controller 300 is adapted for communication with a first external device 320 and a second external device 330, which may comprise receiving, by the internal computing unit, an update or configuration to the control program from the first external device, wherein the update is received using a first communication channel; installing, by the internal computing unit 306, the update; and transmitting, by the internal computing unit, logging data relating to the receipt of the update or configuration and/or logging data relating to an installation of the update to the second external device 330 using the second communication channel; wherein the first and the second communication channels are different communication channels. By using a first and a second communication channels, in comparison to only using one, the security of the updating may be improved as any attempts to update the control program will be logged via the second communication channel, and thus, increasing the chances of finding incorrect or malicious update attempts.

The update or configuration comprises a set of instructions for the control program, and may, for examples comprise a set of predefined program steps as described above. The configuration or update may comprise a value for a predetermined parameter.

In some examples, the method further comprises confirming, by a user or by an external control unit, that the update or configuration is correct based on the received logging data.

The logging data may be related to the receipt of the update or configuration, and the controller 300 is configured to install the update or configuration in response to receipt of a confirmation that the logging data relates to a correct set of instructions. In this way, the controller 300 may receive data, transmit a logging entry relating to the receipt, and then install the data in response to a positive verification that the data should be installed.

In another example, or in combination with the one described above, the logging data is related to the installation or the update or configuration. In this example the logging data may be for information purposes only and not affect the installation, or the method may further comprise activating the installation in response to the confirmation that the update or configuration is correct.

If the update or configuration is transmitted to the controller 300 in one or more steps, the verification as described above may be performed for each of the steps.

The method may further comprise, after transmitting the logging data to the second external device, verifying the update via a confirmation from the second external device 330 via the second communication channel.

With reference to FIG. 65a-65c there may further be provided an implantable controller 300. The controller 300 is connected to a sensor 351 wherein the sensor 351 is at least one microphone sensor 351 configured to record acoustic signals. For instance, the controller 300 may be configured to register a sound related to at least one of a bodily function of the patient and a function of the implantable medical device 10. The controller 300 comprises a computing unit 306 configured to derive at least one of a pulse of the patient from the registered sound related to a bodily function, such as information related to the patient swallowing, from the registered sound related to a bodily function. In the alternative, the controller 300 could be configured to derive information related to a functional status of the implantable medical device 10 from the registered sound, such as RPM of the motor. To this end the computing unit 306 may be configured to perform signal processing on the registered sound (e.g. on a digital or analog signal representing the registered sound) so as to derive any of the above mentioned information related to a bodily function of the patient or a function of the implantable medical device 10.

The signal processing may comprise filtering the registered sound signals of the microphone sensor 351.

The implantable controller is placed in an implantable housing for sealing against fluid, and the microphone sensor 351 is placed inside of the housing. Accordingly, the controller and the microphone sensor 351 do not come into contact with bodily fluids when implanted which ensures proper operation of the controller and the microphone sensor 351.

In some implementations, the computing unit 306 is configured to derive information related to the functional status of an active unit 302 of the implantable medical device 10, from the registered sound related to a function of the implantable medical device 10. Accordingly, the computing unit 306 may be configured to derive information related to the functional status of at least one of: a motor, a pump and a transmission of the active unit 302 of the implantable medical device 10, from the registered sound related to a function of the implantable medical device 10.

The controller may comprise a transceiver 303,308 configured to transmit a parameter derived from the sound registered by the at least one microphone sensor 351 using the transceiver 303,308. For example, the transceiver 303,308 is a transceiver configured to transmit the parameter conductively (303) to an external device 320 or wirelessly (308) to an external device 320.

A method of authenticating the implantable medical device 10, the external device 320 or a communication signal or data stream between the external device 320 and the implantable medical device 10 is also described with reference to FIGS. 65a-65c. The method comprises the steps of registering a sound related to at least one of a bodily function and a function of the implantable medical device 10, using the at least one microphone sensor 351, connected to the controller 300. The method could in a first authentication embodiment comprise transmitting a signal derived from the registered sound, using the transceiver 303,308, receiving the signal in the external device 320, using the receiver 323,328 and comparing, in the external device 320, a parameter derived from the received signal with a reference parameter, using the computing unit 306. The method could in a second authentication embodiment comprise receiving a signal in the controller 300, from the external device 320, using the transceiver 323,328 and deriving a reference parameter from the received signal, using the computing unit 306 of the controller 300, and comparing, in the controller 300, a parameter derived from the received signal with the derived reference parameter, using the computing unit 306 of the controller 300.

The methods further comprise the steps of the implantable controller 300 authenticating the external device 320, or the external device 320 authenticating the implantable controller 300, on the basis of the comparison. The registered sound could for example be related to the patient eating.

According to one embodiment described with reference to FIG. 65A-65C, the communication unit or internal controller 300 or control unit 300 comprises a wireless transceiver 308 for communicating wirelessly with an external device, a security module 389, and a central unit, also referred to herein as a computing unit 306, which is to be considered as equivalent. The central unit 306 is configured to be in communication with the wireless transceiver 308, the security module 389 and the active unit 302 i.e. the medical device for stretching the stomach. The wireless transceiver 308 is configured to receive communication from the external device 320 including at least one instruction to the implantable system 10 and transmit the received communication to the central unit or computing unit 306. The central unit or computing unit 306 is configured to send secure communication to the security module 389, derived from the received communication from the external device 200, and the security module 389 is configured to decrypt at least a portion of the secure communication and verify the authenticity of the secure communication. The security module is further configured to transmit a response communication to the central unit or computing unit 306 and the central unit or computing unit is configured to communicate the at least one instruction to the active unit 302. In another embodiment, the security module is configured to communicate the at least one instruction to the active unit 302 directly. In the embodiment shown in FIG. 65A-65C, the at least one instruction is based on the response communication, or a combination of the response communication and the received communication from the external device 320.

In the embodiment shown in FIG. 65A-65C, the security module 389 comprises a set of rules for accepting communication from the central unit or computing unit 306. In the embodiment shown in FIG. 65A-65C, the wireless transceiver 308 is configured to be able to be placed in an off-mode, in which no wireless communication can be transmitted or received by the wireless transceiver 308. The set of rules comprises a rule stipulating that communication from the central unit or computing unit 306 to the security module 389 or to the active unit 302 is only accepted when the wireless transceiver 308 is placed in the off-mode.

In the embodiment shown in FIG. 65A-65C, the set of rules comprises a rule stipulating that communication from the central unit or computing unit 306 is only accepted when the wireless transceiver 308 has been placed in the off-mode for a specific time period.

In the embodiment shown in FIG. 65A-65C, the central unit or computing unit 306 is configured to verify a digital signature of the received communication from the external device 320. The digital signature could be a hash-based digital signature which could be based on a biometric signature from the patient or a medical professional. The set of rules further comprises a rule stipulating that communication from the central unit 306 is only accepted when the digital signature of the received communication has been verified by the central unit 306. The verification could for example comprise the step of comparing the digital signature or a portion of the digital signature with a previously verified digital signature stored in the central unit 306. The central unit 306 may be configured to verify the size of the received communication from the external device and the set of rules could comprise a rule stipulating that communication from the central unit 306 is only accepted when the size of the received communication has been verified by the central unit 306. The central unit could thus have a rule stipulating that communication above or below a specified size range is to be rejected.

In the embodiment shown in FIG. 65A-65C, the wireless transceiver is configured to receive a message from the external device 320 being encrypted with at least a first and second layer of encryption. The central unit 306 the decrypts the first layer of decryption and transmit at least a portion of the message comprising the second layer of encryption to the security model 389. The security module 389 then decrypts the second layer of encryption and transmits a response communication to the central unit 306 based on the portion of the message decrypted by the security module 389 or transmits the decrypted data to the active unit 302.

In the embodiment shown in FIG. 65A-65C, the central unit 306 is configured to decrypt a portion of the message comprising a digital signature, such that the digital signature can be verified by the central unit 306, also the central unit 306 is configured to decrypt a portion of the message comprising message size information, such that the message size can be verified by the central unit 306.

In the embodiment shown in FIG. 65A-65C, the central unit 306 is configured to decrypt a first and second portion of the message, and the first portion comprises a checksum for verifying the authenticity of the second portion.

In the embodiment shown in FIG. 65A-65C, the response communication transmitted from the security module 389 comprises a checksum, and the central unit 306 is configured to verify the authenticity of at least a portion of the message decrypted by the central unit 306 using the received checksum, i.e. by adding portions of the message decrypted by the central unit 306 and comparing the sum to the checksum.

In the embodiment shown in FIG. 65A-65C, the set of rules further comprise a rule related to the rate of data transfer between the central unit 306 and the security module 389. The rule could stipulate that the communication should be rejected or aborted if the rate of data transfer exceeds a set maximum rate of data transfer, which may make it harder for unauthorized persons to inject malicious code or instructions to the medical implant.

In the embodiment shown in FIG. 65A-65C, the security module 389 is configured to decrypt a portion of the message comprising the digital signature being encrypted with the second layer of encryption, such that the digital signature can be verified by the security module 389. The security module 389 then transmits a response communication to the central unit 306 based on the outcome of the verification, which can be used by the central unit 306 for further decryption of the message or for determining if instructions in the message should be communicated to the active unit 302. Alternatively, the security module determines at least one instruction for the active unit 302 based on the message, and transmits the message to the active unit 203 directly.

In the embodiment shown in FIG. 65A-65C, the central unit 306 is only capable of decrypting a portion of the received communication from the external device 320 when the wireless transceiver 308 is placed in the off-mode. In the alternative, or as an additional layer of security, the central unit 306 may be limited such that the central unit 306 is only capable of communicating instructions to the active unit 302 of the implantable medical device 10 when the wireless transceiver 308 is placed in the off-mode. This ensures that no attacks can take place while the central unit 306 is communicating with the active unit 301.

In the embodiment shown in FIG. 65A-65C, the implantable controller 300 is configured to receive, using the wireless transceiver 308, a message from the external device 320 comprising a first un-encrypted portion and a second encrypted portion. The implantable controller 300 (e.g. the central unit 306 or the security module 389) then decrypts the encrypted portion, and uses the decrypted portion to verify the authenticity of the un-encrypted portion. As such, computing power and thereby energy can be saved by not encrypting the entire communication, but rather only the portion required to authenticate the rest of the message (such as a checksum and/or a digital signature)

In the embodiment shown in FIG. 65A—65C, the central unit 306 is configured to transmit an encrypted portion to the security module 389 and receive a response communication from the security module 389 based on information contained in the encrypted portion being decrypted by the security module. The central unit 306 is then configured to use the response communication to verify the authenticity of the un-encrypted portion. The un-encrypted portion could comprise at least a portion of the at least one instruction to the implantable medical device 306. Alternatively, the central unit 306 is configured to transmit an encrypted portion and an unencrypted portion to the security module 389 and the security module 398 decrypts the encrypted portion and, using the decrypted portion, verifies the authenticity of the un-encrypted portion.

In the embodiment shown in FIG. 65A-65C, the implantable controller 300 is configured to receive, using the wireless transceiver 308, a message from the external device 320 comprising information related to at least one of: a physiological parameter of the patient and a physical or functional parameter of the implanted system 10, and use the received information to verify the authenticity of the message. The physiological parameter of the patient could be a parameter such as a parameter based on one or more of: a temperature, a heartrate and a saturation value.

The physical or functional parameter of the implanted system 10 could comprise at least one of a current setting or value of the active unit 302, a prior instruction sent to the implantable system 10 or an ID of the implanted system 10.

The portion of the message comprising the information related to the physiological parameter of the patient and/or physical or functional parameter of the implanted system 10 could be encrypted, and the central unit 306 may be configured to transmit the encrypted portion to the security module 389 and receive a response communication from the security module 389 based on the information having been decrypted by the security module 389.

In the embodiment shown in FIG. 65A-65C, the security module 389 is a hardware security module comprising at least one hardware-based key. The security module 389 may have features that provide tamper evidence such as visible signs of tampering or logging and alerting. It may also be so that the security module 389 is “tamper resistant”, which makes the security module 389 inoperable in the event that tampering is detected. For example, the response to tampering could include deleting keys is tampering is detected. The security module 389 could comprise one or more secure cryptoprocessor chip. The hardware-based key(s) in the security module 389 could have a corresponding hardware-based key placeable in the external device 320. The corresponding external hardware-based key could be placed on a key-card connectable to the external device 320.

In one embodiment, the security module 387 and the central unit 309 are both comprised in a multi-processor, wherein the security module 387 runs on a first processor and the central unit runs on second processor, different from the first.

In alternative embodiments, the security module 389 is a software security module comprising at least one software-based key, or a combination of a hardware and software-based security module and key. The software-based key may correspond to a software-based key in the external device 320. The software-based key may correspond to a software-based key on a key-card connectable to the external device 320.

In the embodiment shown in FIG. 65A-65C, the external device 320 is a handheld external device, however, in alternative embodiments, the external device may be a remote external device or a cloud based external device

In the embodiment shown in FIG. 65A-65C, the at least one instruction to the implantable system 10 comprises an instruction for changing an operational state of the implantable system 10, including an operational state of the active unit 302, i.e. medical device for stretching the stomach.

In the embodiment shown in FIG. 65A-65C, the wireless transceiver 308 is configured to communicate wirelessly with the external 320 device using electromagnetic waves at a frequency below 100 kHz, or more specifically below 40 kHz. The wireless transceiver 308 is thus configured to communicate with the external device 320 using “Very Low Frequency” communication (VLF). VLF signals have the ability to penetrate a titanium housing of the implantable system 10, such a titanium housing of the remote unit, such that the electronics of the implantable system 10 can be completely encapsulated in a titanium housing.

The wireless transceiver 308 is configured to communicate wirelessly with the external device 320 using a first communication protocol and the central unit 306 is configured to communicate with the security module 389 using a second, different, communication protocol. This adds an additional layer of security as security structures could be built into the electronics and/or software in the central unit 106 enabling the transfer from a first to a second communication protocol. The wireless transceiver 308 may be configured to communicate wirelessly with the external device using a standard network protocol, which could be one of an RFID type protocol, a WLAN type protocol,a Bluetooth (BT) type protocol,a BLE type protocol,an NFC type protocol, a 3G/4G/5G type protocol, and a GSM type protocol. In the alternative, or as a combination, the wireless transceiver 308 could be configured to communicate wirelessly with the external device 320 using a proprietary network protocol. The wireless transceiver 308 could comprises a Ultra-Wide Band (UWB) transceiver and the wireless communication between the implantable controller 300 and the external device 320 could thus be based on UWB. The use of UWB technology enables positioning of the external device 320 which can be used by the implanted system 10 as a way to establish that the external device 320 is at a position which the implanted system 10 and/or the patient can acknowledge as being correct, e.g. in the direct proximity to the implanted system 10 and/or the patient, such as within reach of the patient and/or within 1 or 2 meters of the implanted system 10. In the alternative, a combination of UWB and BT could be used, in which case the UWB communication can be used to authenticate the BT communication, as it is easier to transfer large data sets using BT.

Embodiments relating to an implantable medical device 10 having a controller 300 having a processor 306 with a sleep mode and an active mode will now be described with reference to FIG. 65d. The implant, the internal communication unit and the external device(s) may have the features described above with reference to FIGS. 65a-65c.

In an embodiment in which the controller 300 comprises a processor 306 having a sleep mode and an active mode, the controller 300 comprises or is connected to a sensor 150 and a processing unit 306 having a sleep mode and an active mode. The sensor 150 is configured to periodically measure a physical parameter of the patient, and the controller 300 is further configured to, in response to a sensor measurement preceding a predetermined value, setting the processing unit 306 in an active mode. That is, the controller 300 may “wake up″ or be set in an active mode in response to a measurement from, for example, the body. A physical parameter of the patient could for example be a local or systemic temperature, saturation/oxygenation, blood pressure or a parameter related to an ischemia marker such as lactate.

By sleeping mode it is meant a mode with less battery consumption and/or processing power used in the processing unit 306, and by “active mode” it may be meant that the processing unit 306 is not restricted in its processing.

The sensor 150 may, for example, be a pressure sensor. The pressure sensor may be adapted to measure a pressure in an organ of a patient, a reservoir of the implant or a pressure exerted by at least one member. The sensor 150 may be an analog sensor or a digital sensor, i.e. a sensor 150 implemented in part in software. In some examples, the sensor is adapted to measure one or more of a battery or energy storage status of the implantable medical device 10 and a temperature of the implantable medical device 10. In this way, the sensor 150 may periodically sense a pressure of the implantable medical device 10 or of the patient, and set the processing unit 306 in an active mode if the measured pressure is above a predetermined value. Thus, less power, i.e. less of for example a battery or energy storage comprised in the implant, may be used, thereby prolonging the lifetime of the implantable medical device 10 or increasing the time between charging occasions of the implantable medical device 10.

In some examples, the processor 306, when in set in the active mode, may cause a sensation generator 381 connected to the implant, comprised in the implantable medical device 10 or comprised in an external device 320, 330, to generate a sensation detectable by a sense of the patient. For example, the processor may cause the sensation generator to generate a sensation in response to a measure battery status, for example that the battery is above or below a predetermined level, that a measured pressure is above or below a predetermined level, or that another measured parameter has an abnormal value, i.e. less than or exceeding a predetermined interval or level. The sensation generator has been described in further detail earlier in this description.

The processing unit 306 may be configured to perform a corrective action in response to a measurement being below or above a predetermined level. Such a corrective action may, for example, be increasing or decreasing a pressure, increasing or decreasing electrical stimulation, increasing or decreasing power.

The controller 300 may comprise a signal transmitter 320 connected to the processing unit, and wherein the processing unit is configured to transmit data relating to the measurement via the transceiver 308 of the controller 300 or an additional internal signal transmitter 392. The transmitted data may be received by an external device 320.

The external device may have an external communication unit 390. The external device 320 may comprise a signal provider 380 for providing a wake signal to the controller 300. In some examples, the signal provider comprises a coil or magnet 371 for providing a magnetic wake signal.

The controller 300 may implement a corresponding method for controlling an implantable medical device 10 when implanted in a patient. The method comprises measuring, with a sensor of the controller 300 connected to or comprised in the controller 300, a physiological parameter of the patient or a parameter of the implantable medical device 10, and, in response to a sensor measurement having an abnormal value, setting, by the controller 300, a processor 306 of the controller 300 from a sleep mode to an active mode. The measuring may be carried out periodically. By “abnormal value” it may be meant a measured value exceeding or being less than a predetermined value, or a measured value being outside a predetermined interval. The method may further comprise generating, with a sensation generator 381 as described above, a sensation detectable by the patient. In some examples, the generating comprises requesting, by the processor, the sensation generator 381 to generate the sensation.

The method may further comprise to perform a medical intervention in response to a sensor measurement having an abnormal value, preferably after the processing unit has been set in the active mode.

A system comprising an implantable medical device 10 having a controller 300 having a sleep mode and an active mode will now be described with reference to FIG. 65d. In one embodiment, the controller 300 comprises a sensor 150 adapted to detect a magnetic field and a processing unit 306 having a sleep mode and an active mode, now described with reference to FIGS. 65a-65c. The external control unit 320 comprises a signal provider 380 adapted to provide a magnetic field detectable by the internal sensor 150. The controller 300 is further configured to, in response to a detected magnetic field exceeding a predetermined value, setting the processing unit 306 in an active mode. In this way, the external device 320 may cause a sleeping controller 300 or processor 306 to “wake up″.

The sensor 150 may, for example, be a hall effect sensor, a fluxgate sensor, an ultra-sensitive magnetic field sensor, a magneto-resistive sensor, an AMR or GMR sensor, or the sensor may comprise a third coil having an iron core.

The magnetic field provider 380 may have an off state, wherein it does not provide any magnetic field, and an on state, wherein it provides a magnetic field. For example, the magnetic field provider 380 may comprise a magnet 371, a coil 371, a coil having a core 371, or a permanent magnet 371. In some embodiments, the magnetic field provider 380 may comprise a shielding means for preventing a magnet 371 or permanent magnet 371 from providing a magnetic field in the off state. In order to provide a substantially even magnetic field, the magnetic field provider may comprise a first and a second coil arranged perpendicular to each other.

After the processing unit 306 has been set in an active mode, i.e. when the processing unit 306 has been woken, the implant may determine a frequency for further communication between the controller 300 and the external device 320. The controller 300 may thus comprise a frequency detector 391 for detecting a frequency for communication between the controller 300 and the second communication unit 390. The frequency detector 391 is, for example, an antenna. The external device 320 may comprise a frequency indicator 372, for transmitting a signal indicative of a frequency. The frequency indicator 372, may, for example, be a magnetic field provider capable of transmitting a magnetic field with a specific frequency. In some examples the frequency indicator is comprised in or the same as the magnetic field provider 371. In this way, the frequency signal is detected using means separate from the sensor, and can, for example, be detected using a pin on a chip.

Alternatively, the controller 300 and the external device 320 may communicate using a predetermined frequency or a frequency detected by means defined by a predetermined method according to a predetermined protocol to be used for the communication between the controller 300 and the external device 320.

In some embodiments, the sensor 150 may be used for the communication. The communication may in these embodiments be performed with such that a frequency of the magnetic field generated by the coil is 9-315 kHz, or the magnetic field generated by the coil is less than or equal to 125 kHz, preferably less than 58 kHz. The frequency may be less than 50 Hz, preferably less than 20 Hz, more preferably less than 10 Hz, in order to be transmittable through a titanium box.

In some embodiments, the controller 300 comprises a receiver unit 392, and the internal control unit and the external control unit are configured to transmit and/or receive data via the receiver unit 392 via magnetic induction. The receiver unit 392 may comprise a high-sensitivity magnetic field detector, or the receiver unit may comprise a fourth coil for receiving the magnetic induction.

The system may implement a method for controlling a medical implant implanted in a patient.

The method comprises monitoring for signals by a sensor 150 comprised in the controller 300 communicatively coupled to the active unit 302, providing, from a signal provider 380 comprised in an external device 320, a wake signal, the external device 320 being adapted to be arranged outside of the patient's body, and setting, by the controller 300 and in response to a detected wake signal WS, a mode of a processing unit 306 comprised in the internal control unit from a sleep mode to an active mode.

The method may also comprise detecting, using a frequency detector 391, a frequency for data communication between the controller 300 and a second communication unit 390 being associated with the external device 320. The frequency detector 391 is communicatively coupled to the controller 300 or the external device 320. The detection may be performed using a detection sequence for detecting the frequency. This detection sequence may, for example, be a detection sequence defined in the protocol to be used for communication between the controller 300 and the second communication unit 390. Potential protocols that may be used for communication between the controller 300 and the external device 320 has been described earlier in this description. Thus, the method may comprise determining, using the frequency detector 391, the frequency for data communication, and initiating data communication between the controller 300 and the second communication unit 390. The data communication can, for example, comprise one or more control instructions for controlling the implantable medical device 10 transmitted from the external device 320, or, for example, comprise data related to the operation of the implantable medical device 10 and be transmitted from the controller 300.

In some examples, the implantable medical device may comprise or be connected to a power supply for powering the implantable medical device 10. This will now be described with reference to FIG. 65e. The medical device, the internal control unit, and the external device(s) may comprise all elements described above with reference to FIGS. 65a-65c and FIG. 65d. The power supply may comprise an implantable energy storage unit 40 for providing energy to the medical device, an energy provider 397 connected to the implantable energy storage unit 40 and connected to an energy consuming part of the implantable medical device 10, the energy provider 397 being configured to store energy to provide a burst of energy to the energy consuming part, wherein the energy provider 397 is configured to be charged by the implantable energy storage unit 40 and to provide the energy consuming part with electrical power during startup of the energy consuming part.

Alternatively, the implantable medical device 10 may comprise a first implantable energy storage unit 40 for providing energy to an energy consuming part of the implantable medical device 10, a second implantable energy storage unit 397 connected to the implantable energy storage unit 40 and connected to the energy consuming part, wherein the second implantable energy storage unit 397 is configured to be charged by the implantable energy storage unit 40 and to provide the energy consuming part with electrical power during startup of the energy consuming part. The second implantable energy storage unit 397 has a higher energy density than the first implantable energy storage unit 40. By having a “higher energy density” it may be meant that the second implantable energy storage unit 397 has a higher maximum energy output per time unit than the first implantable energy storage unit 40. The second energy storage 397 may be an energy provider as discussed below.

The energy consuming part may be any part of the implantable medical device 10, such as a motor for powering the hydraulic pump, a valve, a processing or computing unit, a communication unit, a device for providing electrical stimulation to a tissue portion of the body of the patient, a CPU for encrypting information, a transmitting and/or receiving unit for communication with an external unit (not shown as part of the energy consuming part in the drawings, that is, the communication unit may be connected to the energy storage unit 40 and to the energy provider 397), a measurement unit or a sensor, a data collection unit, a solenoid, a piezo-electrical element, a memory metal unit, a vibrator, a part configured to operate a valve comprised in the medical device, or a feedback unit.

In this way, an energy consuming part requiring a quick start or an energy consuming part which requires a high level or burst of energy for a start may be provided with sufficient energy. This may be beneficial as instead of having an idle component using energy, the component may be completely turned off and quickly turned on when needed. Further, this may allow the use of energy consuming parts needing a burst of energy for a startup while having a lower energy consumption when already in use. In this way, a battery or an energy storage unit having a slower discharging (or where a slower discharging is beneficial for the lifetime or health of the battery) may be used for the implant, as the extra energy needed for the startup is provided by the energy provider.

Energy losses may occur in a battery or energy storage unit of an implant if the battery or energy storage unit is discharged too fast. These energy losses may for example be in the form of heat, which may damage the battery or energy storage unit. By the apparatus described in these examples, energy may be provided from the battery or energy storage unit in a way that does not damage the battery or energy storage unit, which may improve the lifetime of the battery or energy storage unit and thereby the lifetime of the medical device.

In some examples, the discharging from the implantable energy storage unit 40 during startup of the energy consuming part is slower than the energy needed for startup of the energy consuming part, i.e. the implantable energy storage unit 40 is configured to have a slower discharging than the energy needed for startup of the energy consuming part. That is, there is a difference between the energy needed by the energy consuming part and the energy the implantable energy storage unit 40 is capable of providing without damaging the implantable energy storage unit 40. In other words, a maximum energy consumption of the energy consuming part may be higher than the maximum energy capable of being delivered by the implantable energy storage unit 40 without causing damage to the implantable energy storage unit, and the energy provider 397 may be adapted to deliver an energy burst corresponding to difference between the required energy consumption and the maximum energy capable of being delivered by the implantable energy storage unit 40. The implantable energy storage unit 40 may be configured to store a substantially larger amount of energy than the energy burst provider 397, but may be slower to charge.

The implantable energy storage unit 40 may be any type of energy storage unit suitable for an implant, such as a re-chargeable battery or a solid-state battery, such as a tionyl-chlorid battery. The implantable energy storage unit 40 may be connected to the energy consuming part and configured to power the energy consuming part after it has been started using the energy provider 397.

The energy provider 397 may be any type of part configured to provide a burst of energy for the energy consuming part. In some examples, the energy provider 397 is a capacitor, such as a start capacitor, a run capacitor, a dual run capacitor or a supercapacitor. The energy provider 397 may be connected to the implantable energy storage unit 40 and be adapted to be charged using the implantable energy storage unit 40. In some examples, the energy provider may be a second energy provider 397 configured to be charged by the implantable energy storage unit 40 and to provide the energy consuming part with electrical energy. The implantable medical device 10 may further comprising a temperature sensor for sensing a temperature of the capacitor and the temperature sensor may be integrated or connected to the controller 300 such that the sensed temperature can be used as input for controlling the implantable medical device 10 or as feedback to be sent to an external device 320.

A corresponding method for powering a medical device may also be contemplated. The method comprises the steps of initiating an energy consuming part 302 of the implant, the energy consuming part being connected to an implantable energy storage unit 40, providing an initial burst of energy to the energy consuming part using an energy provider 397 connected to the implantable energy storage unit 40 and to the energy consuming part 302, the energy provider 397 being adapted to provide a burst of energy to the energy consuming part, and subsequently powering the energy consuming part 302 using the implantable energy storage unit 40.

In some examples, a maximum energy consumption of the energy consuming part is higher than the maximum energy capable of being delivered by the implantable energy storage unit 40 without causing damage to the implantable energy storage unit 40, and the energy provider 397 is adapted to deliver an energy burst corresponding to difference between the required energy consumption and the maximum energy capable of being delivered by the implantable energy storage unit 40.

The method may further comprise the step of charging the energy provider 397 using the implantable energy storage unit 40.

Initiating an energy consuming part 302 may comprise transitioning a control unit of the medical device from a sleep mode to an operational or active mode.

The implantable energy storage unit 40 may be adapted to be wirelessly charged and the implantable energy storage unit may be connected to an internal charger 395 for receiving wireless energy from an external device 320 via an external charger 396, and the method may comprise wirelessly charging the implantable energy storage unit 40. In some examples, the method comprises controlling a receipt of electrical power from an external energy storage unit at the internal charger 395. The internal energy storage unit 40 may be charged via the receipt of a transmission of electrical power from an external energy storage unit 396 by the internal charger 395.

The embodiments described herein may advantageously be combined. For example, all the embodiments relating to the communication and controlling of the medical device may be combined with the embodiments relating to the programming of the implant, the methods and systems for improving energy consumption or the power supply. The embodiments relating to the programming of the medical device may be combined with any of the embodiments relating to improving the energy consumption or the power supply. The embodiments relating to the power supply may be combined with the methods and systems for improving the energy consumption.

A computer program product of, or adapted to be run on, an internal computing unit or an external device is also provided, which comprises a computer-readable storage medium with instructions adapted to make the internal computing unit and/or the external device perform the actions as described in any embodiment or example above.

FIG. 65f and FIG. 65fa shows one embodiment of a system for charging, programming and communicating with the controller 300 of the implanted medical device 100. FIGS. 65f and 65fa further describes the communication and interaction between different external devices which may be devices held and operated by the patient, by the health care provider (HCP) or by the Dedicated Data Infrastructure (DDI), which is an infrastructure supplier for example by the manufacturer of the implanted medical device 100 or the external devices 320′,320″,320″′. The system of the embodiment of FIG. 65f and 65fa comprises three external devices 320′,320″,320″′ capable of communicating with the controller 300. The basic idea is to ensure the security of the communication with, and the operation of, the medical device 100 by having three external devices 320′,320″,320″′ with different levels of authority. The lowest level of authority is given to the patient operated remote control 320″. The remote control external device 320″ is authorized to operate functions of the implanted medical device 100 via the implanted controller 300, on the basis of patient input. The remote control 320″ is further authorized to fetch some necessary data from the controller 300. The remote control 320″ is only capable of operating the controller 300 by communicating with the software currently running on the controller 300, with the currently settings of the software. The next level of authority is given to the Patient External Interrogation Device (P-EID) 320″′, which is a charging and communication unit which is held by the patient but is partially remotely operated by the Health Care Provider (HCP) (Usually a medical doctor with the clinic providing the treatment with help of the implanted medical device 100). The P-EID 320″′ is authorized to make setting changes by selecting pre-programmed steps of the software or hardware running on the controller 300 of the implanted medical device 100. The P-EID is remotely operated by the HCP, and receives input from the HCP, via the DDI. The highest level of authority is given to the HCP-EID 320′ and its controller the HCP Dedicated Display Device (DDD). The HCP-EID 320′ is a charging and communication unit which is held by the HCP physically at the clinic of the HCP. The HCP-EID 320′ is authorized to freely alter or replace the software running on the controller 300, when the patient is physically in the clinic of the HCP. The HCP-EID 320′ is controlled by the HCP DDD, which either acts on a “webview” portal from the HCP-EID or is a device closed down to any activities (which may include the absence of an internet connection) other than controlling and communicating with the HCP-EID. The webview portal does not necessarily mean internet based or HTML-protocol and the webview portal may be communicated over other communicating protocols such as Bluetooth or any other type of standard or proprietary protocol. The HCP DDD may also communicate with the HCP-EID over a local network or via Bluetooth or other standard or proprietary protocols.

Starting from the lowest level of authority, the patient remote control external device 320″ comprises a wireless transceiver 328 for communicating with the implanted medical device 100.

The remote control 320″ is capable of controlling the operation of the implanted medical device 100 via the controller 300, by controlling pre-set functions of the implantable medical device 100, e.g. for operating an active portion of the implanted medical device 100 for performing the intended function of the implanted medical device 100. The remote control 320″ is able communicate with implanted medical device 100 using any standard or proprietary protocol designed for the purpose. In the embodiment shown in FIG. 65f and 65fa, the wireless transceiver 328 comprises a Bluetooth (BT) transceiver, and the remote control 320″ is configured to communicate with implanted medical device 100 using BT. In an alternative configuration, the remote control 320″ communicates with the implanted medical device 100 using a combination of Ultra-Wide Band (UWB) wireless communication and BT. The use of UWB technology enables positioning of the remote control 320″ which can be used by the implanted medical device 100 as a way to establish that the remote control 320″ is at a position which the implanted medical device 100 and/or the patient can acknowledge as being correct, e.g. in the direct proximity to the medical device 100 and/or the patient, such as within reach of the patient and/or within 1 or 2 meters of the implanted medical device 100.

UWB communication is performed by the generation of radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses. A UWB radio system can be used to determine the “time of flight” of the transmission at various frequencies. This helps overcome multipath propagation, since some of the frequencies have a line-of-sight trajectory, while other indirect paths have longer delay. With a cooperative symmetric two-way metering technique, distances can be measured to high resolution and accuracy. UWB is useful for real-time location systems, and its precision capabilities and low power make it well-suited for radio-frequency-sensitive environments, such as health care environments.

In embodiments in which a combination of BT and UWB technology is used, the UWB technology may be used for location-based authentication of the remote control 320″, whereas the communication and/or data transfer could take place using BT or any other way of communicating different from the UWB. The UWB signal could in some embodiments also be used as a wake-up signal for the controller 300, or for the BT transceiver, such that the BT transceiver in the implanted medical device 100 can be turned off when not in use, which eliminates the risk that the BT is intercepted, or that the controller 300 of the implanted medical device 100 is hacked by means of BT communication. In embodiments in which a BT (or alternatives)/UWB combination is used, the UWB connection may be used also for the transmission of data. In the alternative, the UWB connection could be used for the transmission of some portions of the data, such as sensitive portions of the data, or for the transmission of keys for the unlocking of encrypted communication sent over BT.

The remote control 320″ comprises computing unit 326 which runs a software application for communicating with the implanted medical device 100. The computing unit 326 can receive input directly from control buttons 335 arranged on the remote control 320″ or may receive input from a control interface 334i displayed on a patient display device 334 operated by the patient. In the embodiments in which the remote control 320″ receives input from a control interface 334i displayed on the patient display device 334 operated by the patient, the remote control 320″ transmits the control interface 334i in the form of a web-view portal, i.e. a remote interface that run in a sandbox environment on the patient's display device 334. A sandbox environment means that it runs on the display device 334 but only displays what is presented from the remote control and can only use a tightly controlled set of commands and resources, such as storage and memory space as well as network access, the ability to inspect the host system and read or write from other input devices connected to the display device 334 is extremely limited. Any action or command generated by the patient display device is like controlling a webpage. All acting software is located on the remote control that only displays its control interface onto the patient display unit. The computing unit 326 is further configured to encrypt the control interface before transmission to the patient display device 334, and encrypt the control commands before transmission to the implanted medical device 100. The computing unit 326 is further configured to transform the received user input into control commands for wireless transmission to the implantable medical device 100.

The patient's display device 334 could for example be a mobile phone, a tablet or a smart watch. In the embodiment shown in FIG. 65f and 65fa, the patient's display device 334 communicates with the remote control 320″ by means of BT. The control interface 334i in the form of a web-view portal is transmitted from the remote control 320″ to the patient's display device 334 over BT. Control commands in the form of inputs from the patient to the control interface 334i is transmitted from the patient's display device 334 to the remote control 320″, providing input to the remote control 320″ equivalent to the input that may be provided using the control buttons 335. The control commands created in the patient's display device 334 is encrypted in the patient's display device 334 and transmitted to the remote control 320′ using BT or any other communication protocol.

The remote control is normally not connected to the DDI or the Internet to increase security. In addition, the remote control 320″ may in one embodiment have its own private key and in a specific embodiment the remote control 320″ is activated by the patient's private key for a certain time period. This may activate the function of the patient's display device and the remote wed-view display portal supplied by the remote control to the patient's display device.

The patient's private key is supplied in a patient private key device compromising a smartcard that may be inserted or provided close to the remote control 320″ to activate a permission to communicate with the implant 100 for a certain time period.

The patient's display device 334 may (in the case of the display device 334 being a mobile phone or tablet) comprise auxiliary radio transmitters for providing auxiliary radio connection, such as Wi-Fi or mobile connectivity (e.g. according to the 3G, 4G or 5G standards). The auxiliary radio connection(s) may have to be disconnected to enable communication with the remote control 320″. Disconnecting the auxiliary radio connections reduces the risk that the integrity of the control interface 334i displayed on the patient's display device 334 is compromised, or that the control interface 334i displayed on the patient's display device 334 is remote controlled by an unauthorized device.

In alternative embodiments, control commands are generated and encrypted by the patient's display device and transmitted to the DDI 330. The DDI 330 could either alter the created control commands to commands readable by the remote control 320″ before further encrypting the control commands for transmission to the remote control 320″ or could simply add an extra layer of encryption before transmitting the control commands to the remote control 320″, or could simply act as a router for relaying the control commands from the patients' display device 334 to the remote control 320″. It is also conceivable that the DDI 330 adds a layer of end-to-end encryption directed at the implanted medical device 100, such that only the implanted medical device 100 can decrypt the control commands to perform the commands intended by the patient. In the embodiments above, when the patient remote display device 334 is communicating with the DDI, the patient's display device 334 may be configured to only display and interact with a web-view portal provided by a section of the DDI and it is conceivable that the web-view portal is a view of a back-end provided on the DDI 330, and in such embodiments, the patient interacting with the control interface on the patient's display device 334 is equivalent to the patient interacting with an area of the DDI 330.

The patient's display device 334 could have a first and second application related to the implanted medical device 100. The first application is the control application displaying the control interface 334i for control of the implanted medical device 100, whereas the second application is a general application for providing the patient with general information of the status of the implanted medical device 100 or information from the DDI 330 or HCP, or for providing an interface for the patient to provide general input to the DDI 330 or HCP related to the general wellbeing of the patient, the lifestyle of the patient or related to general input from the patient concerning the function of the implanted medical device 100. The second application, which do not provide input to the remote control 320″ and/or the implanted medical device 100 thus handles data which is less sensitive. As such, the general application could be configured to function also when all auxiliary radio connections are activated, whereas switching to the control application which handles the more sensitive control commands and communication with the implanted medical device 100 could require that the auxiliary radio connections are temporarily de-activated. It is also conceivable that the control application is a sub-application running within the general application, in which case the activation of the control application as a sub-application in the general application could require the temporary de-activation of auxiliary radio connections. In the embodiment shown in FIG. 65f, access to the control application requires the use of the optical and/or NFC means of the hardware key 333′ in combination with biometric input to the patient's display device, whereas accessing the general application only requires biometric input to the patient's display device and/or a pin code. In the alternative, a two-factor authentication solution, such as a digital key in combination with a pin code could be used for accessing the general application and/or the control application.

In general, a hardware key is needed to activate the patient display device 334 for certain time period to control the web-view portal of the remote control 320″, displaying the control interface 334i for control of the implanted medical device 100.

In the embodiments in which the patients display device 334 is configured to only display and interact with a web-view provided by another unit in the system, it is conceivable that the web-view portal is a view of a back-end provided on the DDI 330, and in such embodiments, the patient interacting with the control interface on the patient's display device is equivalent to the patient interacting with an area of the DDI 330.

Moving now to the P-EID 320″′. The P-EID 320″′ is an external device used by the patient, patient external device, which communicates with, and charges, the implanted medical device 100. The P-EID 320″′ can be remotely controlled by the HCP to read information from the implanted medical device 100. The P-EID 320″′ controls the operation of the implanted medical device 100, control the charging of the medical device 100, and adjusts the settings on the controller 300 of the implanted medical device 100 by changing pre-defined pre-programed steps and/or by the selection of pre-defined parameters within a defined range, e.g. Just as the remote control 320″, the P-EID 320″′ could be configured to communicate with the implanted medical device 100 using BT or UWB communication or any other proprietary or standard communication method. Since the device may be used for charging the implant, the charging signal and communication could be combined. Just as with the remote control 320″, it is also conceivable to use a combination of UWB wireless communication and BT for enabling positioning of the P-EID 320″ as a way to establish that the P-EID 320″ is at a position which the implanted medical device 100 and/or patient and/or HCP can acknowledge as being correct, e.g. in the direct proximity to the correct patient and/or the correct medical device 100. Just as for the remote control 320″, in embodiments in which a combination of BT and UWB technology is used, the UWB technology may be used for location-based authentication of the P-EID 320″, whereas the communication and/or data transfer could take place using BT. The P-EID 320″ comprises a wireless transmitter/transceiver 328 for communication and also comprises a wireless transmitter 325 configured for transferring energy wirelessly, which may be in the form of a magnetic field or any other signal such as electromagnetic, radio, light, sound or any other type of signal to transfer energy wirelessly to a wireless receiver 395 of the implanted medical device 100. The wireless receiver 395 of the implanted medical device 100 is configured to receive the energy in the form of the magnetic field and transform the energy into electric energy for storage in an implanted energy storage unit 40, and/or for consumption in an energy consuming part of the implanted medical device 100 (such as the operation device, controller 300 etc.). The magnetic field generated in the P-EID 320″′ and received in the implanted medical device 100 is denoted charging signal. In addition to enabling the wireless transfer of energy from the P-EID 320″′ to the implanted medical implant 10, the charging signal may also function as a means of communication. E.g., variations in the frequency of the transmission, and/or the amplitude of the signal may be uses as signaling means for enabling communication in one direction, from the P-EID 320″′ to the implanted medical device 100, or in both directions between the P-EID 320″′ and the implanted medical device 100. The charging signal in the embodiment shown in FIG. 65a and 65fa is a signal in the range 10-65 kHz or 115-140 kHz and the communication follow a proprietary communication signaling protocol, i.e., it is not based on an open standard. In alternative embodiments, BT could be combined with communication using the charging signal, or communication using the charging signal could be combined with an UWB signal. The energy signal could also be used as a carrying signal for the communication signal.

Just as for the remote control 320″, the UWB signal could in some embodiments also be used as a wake-up signal for the controller 300, or for the BT transceiver, such that the BT transceiver in the implanted medical device 100 can be turned off when not in use, which eliminates the risk that the BT is intercepted, or that the controller 300 of the implanted medical device 100 is hacked by means of BT communication. In the alternative, the charging signal could be used as a wakeup signal for the BT, as the charging signal does not travel very far. Also, as a means of location-based authentication, the effect of the charging signal or the RSSI could be assessed by the controller 300 in the implanted medical device 100 to establish that the transmitter is within a defined range. In the BT/UWB combination, the UWB may be used also for transmission of data. In some embodiments, the UWB and/or the charging signal could be used for the transmission of some portions of the data, such as sensitive portions of the data, or for the transmission keys for unlocking encrypted communication sent by BT. Wake-up could be performed with any other signal.

UWB could also be used for waking up the charging signal transmission, to start the wireless transfer of energy or for initiating communication using the charging signal. As the signal for transferring energy has a very high effect in relation to normal radio communication signals, the signal for transferring energy cannot be active all the time, as this signal may be hazardous e.g., by generating heat.

The P-EID 320″′ communicates with the HCP over the Internet by means of a secure communication, such as over a VPN. The communication between the HCP and the P-EID 320″′ is preferably encrypted. Preferably, the communication is sent via the DDI, which may only be relying the information. The communication from the HCP to the implanted medical device 100 may be performed using an end-to-end encryption, in which case the communication cannot be decrypted by the P-EID 320″′. In such embodiments, the P-EID 320″′ acts as a router, only passing on encrypted communication from the HCP to the controller 300 of the implanted medical device 100 (without full decryption). This solution further increases security as the keys for decrypting the information rests only with the HCP and with the implanted medical device 100, which reduces the risk that an unencrypted signal is intercepted by an unauthorized device. The P-EID 320″′ may add own encryption or information, specifically for security reasons. The P-EID 320″′ may hold its own private key and may be allowed to communicate with the implant 100 based on confirmation from the patient's private key, which may be provided as a smartcard to be inserted in a slot of the P-EID 320″′ or hold in close proximity thereto to be read by the P-EID 320″′. These two keys will add a high level of security to the performed communication between the Implant 100 and the P-EID 320″′ since the patient's hardware key in this example on the smartcard may activate and thereby allow the communication and action taken in relation to the implant. The P-EID 320″′ may as previously described change the treatment setting of the implant by selecting pre-programmed steps of the treatment possibilities. Such pre-programmed treatment options may include for example to change:

at least one of the level and time of stretching and when such stretching occur in relation to food intake of a patient for an operable implant for stretching the stomach wall of the patient for creating satiety,

parameters of an implant able to be programmed from outside the body,

parameters of an implant able to be programmed from outside the body with a wireless signal,

parameters of an implant adapted to move fluid inside the body of the patient, such as volume, pumping parameters,

parameters of an implant configured to sense a parameter related to the patient swallowing,

parameters of an implant configured to exercise a muscle with electrical or mechanical stimulation, such as stimulation parameters, amplitude frequency time period etc.,

When the implanted medical device 100 is to be controlled and/or updated remotely by the HCP, via the P-EID 320″′, a HCP Dedicated Device (DD) 332 displays an interface in which predefined program steps or setting values are presented to the HCP. The HCP provides input to the HCP DD 332 by selecting program steps, altering settings and/or values or by altering the order in which pre-defined program steps is to be executed. The instructions/parameters inputted into the HCP DD 332 for remote operation is in the embodiment shown in FIG. 65f routed to the P-EID 320″′ via the DDI 330, which may or may not be able to decrypt/read the instructions. The DDI 330 may store the instructions for a time period to later transfer the instructions in a package of created instructions to the P-EID 320″′. It is also conceivable that an additional layer of encryption is provided to the package by the DDI 330. The additional layer of encryption may be a layer of encryption to be decrypted by the P-EID 330, or a layer of encryption which may only be decrypted by the controller 300 of the implanted medical device 100, which reduces the risk that unencrypted instructions or packages are intercepted by unauthorized devices.

The instructions/parameters are then provided to the P-EID 320″, which then loads the instructions/parameters into the during the next charging/energy transfer to the implanted medical device 100 using any of the signal transferring means (wireless or conductive) disclosed herein.

The Health Care Provider EID (HCP EID) 320′ have the same features as the P-EID 320″ and can communicate with the implanted medical device 100 in the same alternative ways (and combinations of alternative ways) as the P-EID 320″′. However, in addition, the HCP EID 320′ also enables the HCP to freely reprogram the controller 300 of the implanted medical device 100, including replacing the entire program code running in the controller 300. The idea is that the HCP EID 320′ always remain with the HCP and as such, all updates to the program code or retrieval of data from the implanted medical device 100 using the HCP EID 320′ is performed with the HCP and patient present (i.e. not remote). The physical presence of the HCP is an additional layer of security for these updates which may be critical to the function of the implanted medical device 100.

In the embodiment shown in FIG. 65f and 65fa, the HCP communicates with the HCP EID 320′ using a HCP Dedicated Display Device 332 (HCP DDD), which is a HCP display device comprising a control interface for controlling and communicating with the HCP EID 320′. As the HCP EID 320′ always stays physically at the HCP's clinic, communication between the HCP EID 320′ and HCP DDD 332 does not have to be sent over the Internet. Instead, the HCP DDD 332 and the HCP EID 320′ can communicate using one or more of BT, a proprietary wireless communication channel, or a wired connection. The alteration to the programming is then sent to the implanted medical device 100 directly via the HCP EID 320′. Inputting into the HCP DDD 332 for direct operation by means of the HCP EID 320′ is the same as inputting directly into the HCP EID 320′, which then directly transfers the instructions into the implanted medical device 100.

In the embodiment shown in FIG. 65f and 65fa, both the patient and the HCP has a combined hardware key 333′,333″. The combined keys 333′,333″ comprises a hardware component comprising a unique circuitry (providing the highest level of security), a wireless NFC-transmitter 339 for transmitting a specific code (providing mid-level security), and a printed QR-code 344 for optical recognition of the card (providing the lowest level of security). The HCP private key is supplied by a HCP private key device 333″ adapted to be provided to the HCP EID external device via at least one of; a reading slot or comparable for the HCP private key device 333″, an RFID communication or other close distance wireless activation communication to both the HCP EID 320′ and the HCP DDD 332 if used. The HCP DDD 332 will be activated by such HCP private key device 333″, which for example may comprise at least one of, a smartcard, a key-ring device, a watch an arm or wrist band a neckless or any shape device.

The HCP EID external device may comprise at least one of;

a reading slot or comparable for the HCP private key device,

an RFID communication and

other close distance wireless activation communication means

The HCP external device 320′ may further comprise at least one wireless transceiver 328 configured for communication with a data infrastructure server, DDI, through a first network protocol.

A dedicated data infrastructure server, DDI, is in one embodiment adapted to receive commands from said HCP external device 320′ and may be adapted to rely the received commands without opening said commands directed to the patient external device 320″, the DDI 330 comprising one wireless transceiver configured for communication with said patient external device 320″.

The patient EID external device 320″ is in one embodiment adapted to receive the commands relayed by the DDI, and further adapted to send these commands to the implanted medical device 100, which is adapted to receive commands from the HCP, Health Care Provider, via the DDI 330 to change the pre-programmed treatment steps of the implanted medical device 100. The patient EID is adapted to be activated and authenticated and allowed to perform the commands by the patient providing a patient private key device 333′. The patient's private key device is in one embodiment adapted to be provided to the patient external device by the patient via at least one of; a reading slot or comparable for the patient private key device 333′, an RFID communication or other close distance wireless activation communication.

The patient EID external device, in one or more embodiments, comprises at least one of;

a reading slot or comparable for the HCP private key device,

an RFID communication, or

other close distance wireless activation communication

The patient EID external device may in one or more embodiments comprise at least one wireless transceiver configured for communication with the implanted medical device through a second network protocol.

The patient's key 333′ is in the embodiment shown in FIG. 65f and 65fa in the form of a key card having an interface for communicating with the P-EID 320″′, such that the key card could be inserted into a key card slot in the P-EID 320″. The NFC-transmitter 339 and/or the printed QR-code 344 can be used as means for accessing the control interface 334i of the display device 334. In addition, the display device 334 may require a pin-code and/or a biometric input, such as face recognition or fingerprint recognition.

The HCP's key 333″, in the embodiment shown in FIG. 65f and 65fa is in the form of a key card having an interface for communicating with the HCP-EID 320′, such that in one embodiment the key card could be inserted into a key card slot in the HCP-EID 320′. The NFC-transmitter 339 and/or the printed QR-code 344 can be used as means for accessing the control interface of the HCP DDD 332.

In addition, the HCP DDD 332 may require a pin-code and/or a biometric input, such as face recognition or fingerprint recognition.

In alternative embodiments, it is however conceivable that the hardware key solution is replaced by a two-factor authentication solution, such as a digital key in combination with a PIN code or a biometric input (such as face recognition and/or fingerprint recognition). The key could also be a software key, holding similar advance key features, such as the Swedish Bank ID being a good example thereof.

In the embodiment shown in FIG. 65f and 65fa, communication over the Internet takes place over a Dedicated Data Infrastructure (DDI) 330, running on a cloud service. The DDI 330 in this case handles communication between the HCP DDD 332 and the P-EID 320″′. however, the more likely scenario is that the HCP DDD 332 is closed down, such that only the necessary functions of the control application can function on the HCP DDD 332. In the closed down embodiment, the HCP DDD 332 is only able to give the necessary commands to HCP EID 320′ to further update the pre-programmed treatment steps of the Implant 100 via the P-EID 320″′ in direct contact, or more likely indirect contact via the DDI 332. If the patient is present locally, the HCP EID may communicate and act directly on the patient's implant. However, before anything is accepted by the implant, a patient private key device 333′ has to be presented to the P EID 320″′ or HCP EID 320′ for maximum security.

The DDI 330 is logging information of the contact between the HCP and the remote control 320″ via implant feedback data supplied from the implant to P-EID 320″′. Data generated between the HCP and the patient's display device 334, as well as between the HCP and auxiliary devices 336 (such as tools for following up the patient's treatments e.g. a scale in obesity treatment example or a blood pressure monitor in a blood pressure treatment example) are logged by the DDI 330. In some embodiments, although less likely, the HCP DDD 332 may also handle the communication between the patient's display device 334 and the remote control 320″. In FIG. 65fa, the and auxiliary devices 336 is connected to the P-EID as well and can thus provide input from the auxiliary devices 336 to the P-EID which can be used by the P-EID for altering the treatment or for follow up.

In all examples, the communication from the HCP to: the P-EID 320″′, the remote control 320″, the patient's display device 334 and the auxiliary devices 336 may be performed using an end-to-end encryption. In embodiments with end-to-end encryption, the communication cannot be decrypted by the DDI 330. In such embodiments, the DDI 330 acts as a router, only passing on encrypted communication from the HCP to various devices. This solution further increases security as the keys for decrypting the information rests only with the HCP and with the device sending or receiving the communication, which reduces the risk that an unencrypted signal is intercepted by an unauthorized device. The P-EID 320″′ may also only pass on encrypted information.

In addition to acting as an intermediary or router for communication, the DDI 330 collects data on the implanted medical device 100, on the treatment and on the patient. The data may be collected in an encrypted form, in an anonymized form or in an open form. The form of the collected data may depend on the sensitivity of the data or on the source from which the data is collected. In the embodiment shown in FIG. 65f and 65fa, the DDI 330 sends a questionnaire to the patient's display device 334. The questionnaire could comprise questions to the patient related to the general health of the patient, related to the way of life of the patient, or related specifically to the treatment provided by the implanted medical device 100 (such as for example a visual analogue scale for measuring pain). The DDI 330 could compile and/or combine input from several sources and communicate the input to the HCP which could use the provided information to create instructions to the various devices to be sent back over the DDI 330. The data collection performed by the DDI 330 could also be in the form a log to make sure that all communication between the units in the system can be back traced. Logging the communication ensures that all alterations to software or the settings of the software, as well as the frequency and operation of the implanted medical device 100 can be followed. Following the communication enables the DDI 330 or the HCP to follow the treatment and react it something in the communication indicates that the treatment does not provide the intended results or if something appears to be wrong with any of the components in the system. If patient feedback from the patient display device 334 indicates that a new treatment step of the implant is needed, such information must be confirmed by direct contact between HCP and patient.

In the specific embodiment disclosed in FIG. 65f and 65fa, the wireless connections between the different units are as follows. The wireless connection 411 between the auxiliary device 336 and the DDI 330 is based on WiFi or a mobile telecommunication regime or may be sent to the DDI 330 via the P-EID 320″′ and the wireless connection 411 between the auxiliary device 336 and the patient's display device 334 is based on BT or any other communication pathway disclosed herein. The wireless connection 412 between the patient's display device 334 and the DDI 330 is based on WiFi or a mobile telecommunication regime. The wireless connection 413 between the patient's display device 334 and the remote control 320″ is based on BT or any other communication pathway disclosed herein. The wireless connection 414 between the patient remote control 320″ and the implanted medical device 100 is based on BT and UWB or any other communication pathway disclosed herein. The wireless connection 415 between the remote control 320″ and the DDI 330 is likely to not be used, and if present be based on WiFi or a mobile telecommunication regime. The wireless connection 416 between the P-EID 320″′ and the implanted medical device 100 is based on BT, UWB and the charging signal or any other communication or energizing pathway disclosed herein. The wireless connection 417 between the P-EID 320″′ and the DDI 330 is based on WiFi or a mobile telecommunication regime. The wireless connection 418 between the HCP-EID 320′ and the implanted medical device 100 is based on at least one of the BT, UWB and the charging signal. The wireless connection 419 between the P-EID 320″′ and the HCP DD 332 is based on BT or any other communication path disclosed herein. The wireless connection 420 between the HPC-EID 320′ and the DDI 330 is based on WiFi or a mobile telecommunication regime. The wireless connection 421 between the HPC DD 332 and the DDI 330 is normally closed and not used and if so based on WiFi or a mobile telecommunication regime. The wireless connection 422 between the HCP-EID 320′ and the HCP DD 332 is based on at least one of BT, UWB, local network or any other communication path disclosed herein.

The wireless connections specifically described in the embodiment shown in FIG. 65f and 65fa may however be replaced or assisted by wireless connections based on radio frequency identification (RFID), near field communication (NFC), Bluetooth, Bluetooth low energy (BLE), or wireless local area network (WLAN). The mobile telecommunication regimes may for example be 1G, 2G, 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication.

The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

FIG. 65fa also discloses a master private key 333″′ device that allow issuance of new private key device wherein the HCP or HCP admin have such master private key 333″′ device adapted to be able to replace and pair a new patient private key 333′ device or HCP private key device 333″ into the system, through the HCP EID external device 320′.

A system configured for changing pre-programmed treatment settings of an implantable medical device, when implanted in a patient, from a distant remote location in relation to the patient, the system comprising:

FIG. 65fa also discloses a scenario in which at least one health care provider, HCP, external device 320′ is adapted to receive a command from the HCP to change said pre-programmed treatment settings of an implanted medical device 100, further adapted to be activated and authenticated and allowed to perform said command by the HCP providing a HCP private key device 333″. The HCP EID external device 320′ further comprising at least one wireless transceiver 328 configured for communication with a patient EID external device 320″′, through a first network protocol. The system comprises the patient EID external device 320″′, the patient EID external 320″′ device being adapted to receive command from said HCP external device 320′, and to relay the received command without modifying said command to the implanted medical device 100. The patient EID external device 320″′ comprising one wireless transceiver 328. The patient EID 320″′ is adapted to send the command to the implanted medical device 100, to receive a command from the HCP to change said pre-programmed treatment settings of the implanted medical device 100, and further to be activated and authenticated and allowed to perform said command by the patient providing a patient private key 333′ device comprising a patient private key.

Although wireless transfer is primarily described in the embodiment disclosed with reference to FIGS. 65f, 65fa the wireless communication between any of the external device may be substituted for wired communication. Also, some or all of the wireless communication between an external device and the implanted medical device 100 may be substituted for conductive communication using a portion of the human body as conductor.

FIG. 65fb shows a portion of FIG. 65f, in which some of the components have been omitted to outline a specific scenario. In the scenario outlined in FIG. 65fb, the system is configured for changing pre-programmed treatment settings of an implantable medical device 100, when implanted in a patient, from a distant remote location in relation to the patient. The system if FIG. 65fb comprises at least one HCP EID 320′ external device adapted to receive commands from the HCP to change said pre-programmed treatment settings of an implanted medical device 100. The HCP EID 320′ external device is further adapted to be activated and authenticated and allowed to perform said command by the HCP providing a HCP private key device 333″ adapted to be provided to the HCP EID external device 320′.

The private key device 333″ is adapted to be provided to the HCP EID external device 320′ via at least one of: a reading slot or comparable for the HCP private key device 333″, and an RFID communication or other close distance wireless activation communication. The HCP EID external device 320′ comprises at least one of: a reading slot or comparable for the HCP private key device 333″, an RFID communication, and other close distance wireless activation communication or electrical direct contact.

The HCP EID external device 320′ further comprises at least one wireless transceiver 328 configured for communication with a dedicated data infrastructure server (DDI) 330, through a first network protocol.

The system further comprises a dedicated data infrastructure server (DDI) 330, adapted to receive command from said HCP EID external device 320′, adapted to relay the received commands without modifying said command to a patient EID external device 320″′. The dedicated data infrastructure server (DDI) 330 further comprises a wireless transceiver 328 configured for communication with said patient external device. The system further comprises a patient EID external device 320″′ adapted to receive the command relayed by the dedicated data infrastructure server (DDI) 330 and further adapted to send commands to the implanted medical device 100 and further adapted to receive commands from the HCP EID external device 320′ via the dedicated data infrastructure server (DDI) 330 to change said pre-programmed treatment settings of the implanted medical device 100. The patient EID external device 320″′, and further adapted to be activated and authenticated and allowed to perform said command by the patient providing a patient private key device 333′ adapted to be provided to the patient EID external device 320″′ by the patient via at least one of a reading slot or comparable for the patient private key device 333′, an RFID communication or other close distance wireless activation communication or electrical direct contact. The patient EID external device 320″′ further comprises at least one of: a reading slot or comparable for the HCP private key device, an RFID communication and other close distance wireless activation communication or electrical direct contact. The patient EID external device 320″′ further comprises at least one wireless transceiver 328 configured for communication with the implanted medical device 100 through a second network protocol. The implanted medical device 100 is in turn configured to treat the patient or perform a bodily function.

The scenario described with reference to FIG. 65fb may in alternative embodiments be complemented with additional units or communication connections, or combined with any of the scenarios described with reference to FIGS. 65fc-65fe.

FIG. 65fc shows a portion of FIG. 65f, in which some of the components have been omitted to outline a specific scenario. In the scenario outlined in FIG. 65fc, system configured for changing pre-programmed treatment settings of an implantable medical device 100 is disclosed. The changing pre-programmed treatment settings are performed by a health care provider (HCP) in the physical presence of the patient. The system comprises at least one HCP EID external device 320′ adapted to receive commands from the HCP, directly or indirectly, to change said pre-programmed treatment settings in steps of an implantable medical device 100, when implanted. The HCP EID external device 320′ is further adapted to be activated, authenticated, and allowed to perform said command by the HCP providing a HCP private key device 333″ comprising a HCP private key. The HCP private key device in the embodiment of FIG. 65fc, comprises at least one of: a smart card, a keyring device, a watch, a arm or wrist band, a necklace, and any shaped device. The HCP EID external device 320′ is adapted to be involved in at least one of: receiving information from the implant 100, receiving information from a patient remote external device 336, actuating the implanted medical device 100, changing pre-programmed settings, and updating software of the implantable medical device 100, when implanted.

The HCP EID external device 320′ is adapted to be activated, authenticated, and allowed to perform said command also by the patient, the system comprises a patient private key device 333′ comprising a patient private key. The patient private key device 333′ comprising at least one of a smart card, a keyring device, a watch, a arm or wrist band, a necklace, and any shaped device. The HCP private key 333″ and the patients private key are required for performing said actions by the HCP EID external device 320′ to at least one of receive information from the implant 100, to receive information from a patient remote external device 336, to actuate the implanted medical device 100, to change pre-programmed settings, and to update software of the implantable medical device 100, when the implantable medical device is implanted.

FIG. 19c also outlines a scenario in which the system is configured for changing pre-programmed treatment settings in steps of an implantable medical device, when implanted in a patient, by a health care provider, HCP, with the patient on remote on distance, the system comprising: at least one HCP EID external device 320′ adapted to receive a command from the HCP direct or indirect, to change said pre-programmed treatment settings in steps of an implantable medical device, when implanted, wherein the HCP EID external device 320′ is further adapted to be activated, authenticated, and allowed to perform said command by the HCP. The said action by the HCP EID external device 320′ to change pre-programmed settings in the implant 100 and to update software of the implantable medical device 100, when the implantable medical device 100 is implanted, is adapted to be authenticated by a HCP private key device 333″ and a patient private key device 333′.

The scenario described with reference to FIG. 65fc may in alternative embodiments be complemented with additional units or communication connections, or combined with any of the scenarios described with reference to FIGS. 65fb, or 65fd-65fe.

FIG. 65fd shows a portion of FIG. 65f, in which some of the components have been omitted to outline a specific scenario. In the scenario outlined in FIG. 65fd, a system configured to change pre-programmed and pre-selected treatment actions of an implantable medical device 100 by command from the patient is described. The system comprises an implantable medical device 100, a patient remote external device 320″, and a wireless transceiver 328 configured for communication with the implantable medical device 100, when the medical device is implanted, through a second network protocol, The system further comprises a remote display portal interface 334i configured to receive content delivered from the patient remote external device 320″ to expose buttons to express the will to actuate the functions of the implanted medical device 100 by the patient through the patient remote external device 320″. The remote external device 320″ is further configured to present the display portal remotely on a patient display device 334 allowing the patient to actuate the functions of the implanted medical device 100 through the display portal of the patient remote external device 320″ visualised on the patient display device 334. In FIG. 65fd, a further wireless connection 423 between the patient remote external device 320″ and the patient EID external device 320″′ is provided. This further wireless connection 423 could be a wireless connection according to any one of the wireless signaling methods and protocols described herein, and the communication can be encrypted.

FIG. 65fd further shows a scenario in which the external system comprises a first external device in the form of the HCP EID external device 320′ and a second external device in the form of patient EID external device 320″′. The HCP EID external device 320′ and the patient EID external device 320″′ have a wireless or wired connection 416′ to each other and external system is configured for providing remote instructions to the implantable medical device 100. The HCP EID external device 320′ or the patient EID external device 320″′ is configured to, derive a checksum from the instructions that will be sent to the implant and electronically sign the instructions and the checksum using at least one of a patient private key device 333′ or a HCP private key device 333″′. The HCP EID external device 320′ or the patient EID external device 320″′ is then configured to form a data packet from the instructions, the electronic signature and the checksum. In the embodiment shown in FIG. 65fd, the patient EID external device 320″′ comprises a wireless transmitter configured to wirelessly send the data packet to the implantable medical device 100. The HCP EID external device 320′ or the patient EID external device 320″′ may further be configured encrypt the data packet prior to transmission. If the HCP EID external device 320′ creates and signed the instructions, the patient EID external device 320″′ may be configured to transmit the data packet wirelessly to the implantable medical device without changing the data packet and/or without full decryption of the data packet. In the embodiment shown in FIG. 65fd, the patient private key and the HCP private key are placed on a patient private key device 333′ and a HCP private key device 333″′. However, the patient private key and the HCP private key may be placed directly on the HCP EID external device 320′ or the patient EID external device 320″′. Either way, the patient private key and the HCP private key may be placed on the EIDs or the key devices by the manufacturers and may be placed on the EIDs or the key devices in the form of software or hardware.

The key may be a non-extractable key.

In the example when the HCP EID external device 320′ communicates directly with the patient EID external device 320″′, the external system is configured to function without connection to the Internet which greatly reduces the risk that the system is hacked. As the system is not connected to the Internet, the system cannot depend on a synchronized time e.g. for time-out of log-in functionality. As such, the external system is configured to communicate with the implantable medical device 100 independently of time. The authentication and verification may thus be based entirely on the possession of keys. In an alternative embodiment, the log-in of signing functionality offered by the key devices 333″, 333″′ may be complemented or replaced by an input button on one or both of the HCP EID external device 320′ or the patient EID external device 320″′, configured to be used for verifying user presence. I.e., a user presses the input button on request from the HCP EID external device 320′ or the patient EID external device 320″′ and thereby verifies presence.

The implantable medical device 100 is in this embodiment configured to receive remote instructions from the external system by a wireless receiver configured to receive wirelessly transmitted data packets from the external system, i.e. the HCP EID external device 320′ or the patient EID external device 320″′. The implantable medical device 100 is configured to: verify the electronic signature, and use a checksum provided in the data packet to verify the integrity of the instructions.

A verification query operation may further be built into the external system or between the external system and the implantable medical device 100. The verification query operation comprising: transmitting, from the HCP EID external device 320′, the patient EID external device 320″′, or the implantable medical device 100, a query comprising a computational challenge to at least one other of the HCP EID external device 320′, the patient EID external device 320″′, or the implantable medical device 100 and receiving, at the first or second external devices, a response based on the transmitted computational challenge, and verifying at the HCP EID external device 320′, the patient EID external device 320″′, or the implantable medical device 100, the received response. The verification query operation may be in the form of a proof of possession operation comprising: receiving a public key, the public key being associated with a private key, transmitting a computational challenge to the first or second key device, based on the public key received from the first or second key device, receiving a response from the first or second key device based on the possession of the private key in the first or second key device, and verifying that the response based on the possession of the private key matches the query based on a public key. The verification query operation may also be performed between one of the HCP EID external device 320′ or the patient EID external device 320″′ and one of the first and second key devices.

In an alternative authentication or verification method for providing remote instructions from the external system to the implantable medical device 100, the implantable medical device comprises a list of codes and the external system comprises a list of codes. The method comprising encrypting the instructions at the external system using a code from a position on the list of codes, wirelessly sending the encrypted instructions to the implantable medical device, and decrypting, at the implantable medical device, the instructions using a code from a position on the list of codes. The same authentication or verification method may be used for authentication or verification or s signature applied to a communication which may comprise at least one instruction.

The scenario described with reference to FIG. 65fd may in alternative embodiments be complemented with additional units or communication connections, or combined with any of the scenarios described with reference to FIGS. 65fb, 65fc, or 65fe.

FIG. 65fe shows a portion of FIG. 65fa, in which some of the components have been omitted to outline a specific scenario. In the scenario outlined in FIG. 65fe, a system configured for providing information from an implantable medical device 100, when implanted in a patient, from a distant remote location in relation to the patient is described. The system comprises at least one patient EID external device 320″′ adapted to receive information from the implant 100, and adapted to send such information further on to a server or dedicated data infrastructure, DDI, 330. The patient EID external device 320″′ is further adapted to be activated and authenticated and allowed to receive said information from the implanted medical device 100 by the patient providing a private key, The patient private key device comprises the private key adapted to be provided to the patient EID external device 320″′ via at least one of; a reading slot or comparable for the patient private key device, an RFID communication or other close distance wireless activation communication or direct electrical connection, The patient EID external device 320″′ comprises at least one of; a reading slot or comparable for the patient private key device, an RFID communication and other close distance wireless activation communication or direct electrical contact, The patient EID external device 320″′ further comprises at least one wireless transceiver 328 configured for communication with the DDI 330, through a first network protocol.

The scenario described with reference to FIG. 65fe may in alternative embodiments be complemented with additional units or communication connections, or combined with any of the scenarios described with reference to FIGS. 65fb-65fd.

FIG. 65ff shows a portion of FIG. 65fa, in which some of the components have been omitted to outline a specific scenario. In the scenario outlined in FIG. 65ff a system configured for changing pre-programmed treatment settings in steps of an implantable medical device 100, when implanted in a patient, by a health care provider, HCP, either in the physical presence of the patient or remotely with the patient on distance is described. The system comprising at least one HCP EID external device 320′ adapted to receive a command directly or indirectly from the HCP to change said pre-programmed treatment settings in steps of the implantable medical device 100, when implanted, wherein the HCP EID external device 320′ is further adapted to be activated, authenticated, and allowed to perform said command by the HCP providing a HCP private key device comprising a HCP private key, comprising at least one of: a smart card, a keyring device, a watch, a arm or wrist band, a necklace, and any shaped device. The system further comprises a patient private key device comprising a patient private key comprising at least one of: a smart card, a keyring device, a watch, a arm or wrist band, a necklace, and any shaped device. Both the HCP and patient private key is required for performing said action by the HCP EID external device 320′ to change the pre-programmed settings in the implant 100 and to update software of the implantable medical device 100, when the implantable medical device 100 is implanted. The patient private key is adapted to activate, be authenticated, and allowed to perform said command provided by the HCP, either via the HCP EID external device or when the action is performed remotely via a patient EID external device 320′. In the embodiment shown in FIG. 65ff, the communication is routed over the DDI server 330.

The scenario described with reference to FIG. 65ff may in alternative embodiments be complemented with additional units or communication connections, or combined with any of the scenarios described with reference to FIGS. 65fb-65fe.

FIG. 65fg shows an overview of an embodiment of the system, similar to that described with reference to FIG. 65fa, the difference being that the HCP EID and the HCP DDD are combined into a single device.

FIG. 65fh shows an overview of an embodiment of the system, similar to that described with reference to FIG. 65fa, the difference being that the HCP EID 320″′ and the HCP DDD 332 are combined into a single device and the P EID 320″′ and the patient remote control external device 320″ are combined into a single device.

One probable scenario/design of the communication system is for the purpose of changing pre-programmed treatment settings of an implantable medical device, when implanted in a patient, from a distant remote location in relation to the patient. The system comprises at least one health care provider, HCP, external device 320′ adapted to receive a command from the HCP to change said pre-programmed treatment settings of an implanted medical device, further adapted to be activated and authenticated and allowed to perform said command by the HCP providing a HCP private key device 333″ adapted to be provided to an HCP EID external device via at least one of; a reading slot or comparable for the HCP private key device, a RFID communication or other close distance wireless activation communication. The HCP EID external device comprising at least one of: a reading slot or comparable for the HCP private key device, a RFID communication, and other close distance wireless activation communication or electrical direct contact. The HCP EID external device further comprises at least one wireless transceiver configured for communication with a patient EID external device, through a first network protocol, wherein the system comprises the patient EID external device, the patient EID external device being adapted to receive command from said HCP external device, and to relay the received command without modifying said command to the implanted medical device. The patient EID external device comprising one wireless transceiver configured for communication with said patient external device. The patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.

In another scenario, the implantable medical device may be configured to transmit information. Such information may, for example, relate to a function of the implantable medical device, a parameter of the body of the patient, measurements, among others. In that scenario, the implantable medical device may be configured to only transmit such data in response to a received authentication.

The authentication may be received from the patient EID, or from another external device. The implantable medical device may verify that the authenticated device is authorized to request data, for example through a cryptographic verification, which in some examples is based on a key stored at the implantable medical device.

The patient EID (alternatively patient external device) may provide the authentication based on a patient private key provided to the patient EID. The implantable medical device may in that scenario verify that the authentication is based on a patient private key associated with a patient that is authorized to request information from the implant. Based on a valid authorization, the implantable medical device may send data to the patient external device. The data may in some examples be encrypted, for example in any of the ways of encrypting data from the medical implant are described herein. The authorization may be a one-time authorization, an authorization for a predetermined time interval or an authorization that is valid until withdrawn. For example, the authorization may be provided once a day, or at the time of requesting the data from the implantable medical device.

While FIGS. 65F and 65FA-FH and the description thereof discloses different scenarios, also other combinations of authentication or authorization are conceivable. For example, for changing settings of an implantable medical device, it may be required that a health care provider provides an authentication for the changes using their HCP private key. In some examples, an authorization is necessary also from the patient by the patient providing a patient private key device in order for the changes to be accepted by the implantable medical implant. In some examples, the patient may delegate its authorization to the health care provider by using the patient private key.

In some cases, when both an authorization from the health care provider and the patient are required, the health care provider and the patient could be in the same location. To provide an authorization showing that both the health care provider and the patient are at the same location, either the HCP external device or the patient external device may be adapted to receive both the patient private key and the HCP private key in order to authorize a command or a change for the implantable medical implant. Alternatively, the HCP external device or the patient external device may be configured to communicate via a short range communications technology to verify that the other device is present and authenticated before sending the changes to the implantable medical device. This added security may be beneficial, for example, when the medical implant is re-programmed, or software of the implantable medical device is otherwise changed.

In other examples, both an authorization from a patient and from a health care provider may be required, but without the requirement that they are at the same location. In those examples, the authorization may be given using their respective external device. This may be beneficial, for example, when making changes to treatment settings or updating a software is considered to be low risk. Different programs comprised in the implantable medical implant may be considered to have a different risk level associated with them. A risk determination may be programmed into the implantable medical implant as conditions for accepting an update. If the implantable medical implant determines that an update fulfils the conditions, it may install it, otherwise, if the implantable medical implant determines that the conditions are not fulfilled, it may reject the update.

In some examples, it may be sufficient to only require an authorization from at least one of a health care provider and a patient. For example, changes associated with a lower risk, such as changing pre-programmed settings or treatment settings within pre-determined ranges, may be performed using only one authorization.

Although the different scenarios outlined in FIGS. 65fb-65fh are described with specific units and method of signaling, these scenarios may very well be combined with each other or complemented with additional units or communication connections.

As have been discussed before in this application, communication with a medical implant needs to be reliable and secure. For this purpose, it is desirable to have a standalone device as an external remote control (for example described as 320″ in FIGS. 65f-65fh) for the medical implant, such that no other programs or applications run on the same device which may disturb or corrupt the communication to the medical implant. However, the smartphone or tablet (for example described as 334 in FIGS. 65f-65fh) has become an integrated part of everyday life for most people. This means that we almost always have our smartphones at hand. For this reason, it would have been convenient for the patient to communicate with the medical implant directly using the smartphone, such that no additional standalone device would have to be carried. However, as a lot of other applications are running on the smartphone, it does not fulfill the requirement of being a secure and reliable communication tool without interference from other communication. It is therefore desirable to split the tasks of providing secure communication between the external device and the implant from the task of communicating with the Internet and providing a familiar and intuitive user interface. For this purpose, and external device providing secure communication and tamperproof soft and hardware, where the display device allows for intuitive and easy use is provided. In the embodiments described with reference to FIGS. 65g-65k a device fulfilling these combinatory needs will be described in the form of a standalone remote control external device integrated in a housing unit 320″ connectable to a smartphone or another display device 334, such as a smart watch or a tablet.

FIG. 65g shows the housing unit 320″ in an elevated perspective view form the left, and FIG. 65h shows the housing unit 320″ in a plain view from the left. In the embodiment shown in FIG. 65g, the housing unit 320″ has a rectangular shape with rounded edges, having a height 1521 which is more than 1.5 times the width 1522. The housing unit 320″ comprises recess 1525 configured to receive a display device 334, in the form of a smartphone, configured to be fitted in the housing unit 320″ for mechanically, disconnectably connecting the display device 334 to the housing unit 320″. The boundaries of the recess 1525 in the housing unit 320″ forms an edge 1528 configured to encircle the display device 334, when the display device 334 is inserted into the recess 1525. In the embodiment shown in FIG. 65g, the recess 1525 has a depth 1526 configured to allow the display device 334 to be entirely inserted into the recess 1525. As such, the depth 1526 of the recess 1525 exceeds the depth 1531 of the display device 334. In the embodiment shown in FIGS. 65g and 65h, the edge is relatively thin, and has a width 1527 which is in the range ⅛- 1/100 of the width of the display device 334, as such, the housing unit 320″ has a width in the range 1,02-1,25 times the width 1522 of the housing unit 320″. In the same way, the housing unit 320″ has a height 1521 in the range 1,01-1,25 times the height 1521 of the display device 334. In the embodiment shown in FIGS. 65g-65h, the edges 1528 are configured to clasp the display device 334 and thereby mechanically fixate the display device 334 in the housing unit 320″. The minimum bounding box of the housing unit 320″ and the display device 334 when mechanically connected, is no more than, 10% wider, 10% longer or 100% higher, than the minimum bounding box of the display device 334.

For creating a clasping fixation, the edges of the housing unit 320″ is made from an elastic material crating a tension between the edge 1528 and the display device 334 holding the display device 334 in place. The elastic material could be an elastic polymer material, or a thin sheet of elastic metal. For the purpose of further fixating the display device 334 in the housing unit 320″, the inner surface of the edges 1528 may optionally comprise a recess or protrusion (not shown) corresponding to a recess or protrusion of the outer surface of the display device 334. The edges 1528 may in the alterative comprise concave portions for creating a snap-lock clasping mechanical fixation between the housing unit 320″ and the display device 334.

In the embodiment shown in FIGS. 65g and 65h, the housing unit 320″ functions as a remote control for communicating with an implanted medical device, including receiving information from, and providing instructions and updates to, the implanted medical device. Information could be information related to a state of the implanted medical device including any functional parameter of the implanted medical device or could be related to a state of the patient, including any physiological parameter pertaining to the body of the patient (further described on other sections of this disclosure). For the purpose of providing input to the implanted medical device and controlling and updating the functions of the housing unit 320″, the housing unit 320″ comprises a control interface comprising switches in the form of control buttons 335. The control buttons 335 are configured to be used when the external device is disconnected from the display device 334. The control interface further comprises a display 1505, which is a smaller and typically less sophisticated display 1505 than the display of the display device 334. In an alternative embodiment, the control buttons 335 and display 1505 are integrated into a single touch-responsive (touchscreen) display on which the control buttons may be displayed. In the embodiment shown in FIGS. 65g and 65h, one of the control buttons 335 is a control button for activating the implanted medical device and another of the control buttons 335 is a control button for deactivating the implanted medical device. When the display device 334 is attached to the housing unit 320″, the control buttons 335 and the display is covered by the display device 334 and are as such not in an operational state. In the embodiment shown in FIGS. 65g and 65h, the housing unit 320″ is configured to transmit information pertaining to the display of the user interface to the display device 334 and the display device 334 is configured to receive input pertaining to communication to or from the implantable medical device from the patient, and transmit signals based on the received input to the housing unit 320″. The input may be a command to change the operational state of the implantable medical device. The display device 334 comprises a touch screen configured to display the user interface and receive the input from the patient. The display of the display device 334 may comprise one or more OLEDs or IPS LCDs elements. When the display device 334 is connected to the housing unit 320″, the display device 334 is configured to display a control interface which is used to communicate with the housing unit 320″, i.e. providing input to and receiving information from the housing unit 320″. The input provided the housing unit 320″ is then relayed to the implanted medical device—and in the same way information communicated from the implanted medical device to the housing unit 320″ may be relayed or displayed on the display device 334. Having an external device comprising a combination of a housing unit 320″ comprising the communication means for communicating with the implanted medical device and a display device 334 basically only functioning as and Input/Output device connected to the housing unit 320″ makes it possible to have a secure communication between the housing unit 320″ and the display device 334, which is out of reach from the Internet connection of the display device 334, which makes it much harder for an external attacker to get access to any of the vital communication portions of the housing unit 320″. The communication between the housing unit and the display device 334 is very restricted and the only communication allowed from the display device 334 to the housing unit 320″ is input from the patient or a healthcare professional, and authentication parameters created by an authentication application running on the display device 334. The authentication application running on the display device 334 could be a number-generating authenticator or a biometric authenticator for authenticating the patient or health care professional, and the authentication parameters could for example be parameters derived from a facial image or a fingerprint. In the opposite direction, i.e. from the housing unit 320″ to the display device 334, the communication could be restricted to only communication needed for displaying information and/or a graphical user interface on the display device 334. The communication restrictions could for example be based on size of the communication packages or the frequency with which the communication takes place which reduces the risk that an un-authorized person makes multiple attempts to extract information from, or transit information to, the hand-held device.

In the embodiment shown with reference to FIGS. 65g and 65h, the housing unit 320″ comprises a first communication unit providing a wireless connection 413 to the display device 334. The wireless connection 413 is in the embodiment shown in FIGS. 65g and 65h based on NFC, but could in alternative embodiment be based on Bluetooth or any other communication pathway disclosed herein. The housing unit 320″ further comprises a second communication unit providing a wireless connection with the implanted medical device. The wireless communication between the housing unit 320″ and the implanted medical device is in the embodiment shown in FIGS. 65g and 65h based on Bluetooth, but could in alternative embodiments be based on NFC or UWB or any other communication pathway disclosed herein.

As mentioned, in the embodiment shown in FIGS. 65g and 65h, the wireless communication between the housing unit 320″ and the display device 334 is based on NFC, while the wireless communication between the housing unit 320″ and the is based on Bluetooth. As such, the first communication unit of the housing unit 320″ is configured to communicate wirelessly with the display device 334′ using a first communication frequency and the second communication unit of the housing unit 320″ is configured to communicate wirelessly with the implantable medical device using a second different communication frequency. For this purpose, the first communication unit of the housing unit 320″ comprises a first antenna configured for NFC-based wireless communication with the display device 334, and the second communication unit comprises a second antenna configured for Bluetooth-based wireless communication with the implantable medical device. The first and second antennae may be a wire-based antennae or a substrate-based antennae. As such, the first communication unit is configured to communicate wirelessly with the display device 334 on a first frequency and the second communication unit is configured to communicate wirelessly with the implantable medical device using a second different communication frequency. Also, first communication unit of the housing unit 320′ is configured to communicate wirelessly with the display device 334 using a first communication protocol (the NFC-communication protocol), and the second communication unit is configured to communicate wirelessly with the implantable medical device using a second communication protocol (the Bluetooth communication protocol). The first and second communication protocols are different which adds an additional layer of security as security structures could be built into the electronics and/or software enabling the transfer from a first to a second communication protocol.

In an alternative embodiment, the second communication unit may be configured to communicate wirelessly with the implantable medical device using electromagnetic waves at a frequency below 100 kHz, or preferably at a frequency below 40 kHz. The second communication unit may thus be configured to communicate with the implantable medical device using “Very Low Frequency” communication (VLF). VLF signals have the ability to penetrate a titanium housing of the implant, such that the electronics of the implantable medical device can be completely encapsulated in a titanium housing. In yet further embodiments, the first and second communication units may be configured to communicate by means of an RFID type protocol, a WLAN type protocol, a BLE type protocol, a 3G/4G/5G type protocol, or a GSM type protocol.

In yet other alternative embodiments, it is conceivable that the mechanical connection between the housing unit 320″ and the display device 334 comprises an electrical connection for creating a wire-based communication channel between the housing unit 320″ and the display device 334. The electrical connection could also be configured to transfer electric energy from the display device 334 to the housing unit, such that the housing unit 320″ may be powered or charged by the display device 334. A wired connection is even harder to access for a non-authorized entity than an NFC-based wireless connection, which further increases the security of the communication between the housing unit 320″ and the display device 334.

In the embodiment shown with reference to FIGS. 65g and 65h, the display device 334 comprises a first communication unit providing a wireless connection 413 to the housing unit 320″ based on NFC. The display device 334 further comprises a second communication unit providing a wireless connection with a further external device and/or with the Internet. The second external device may be far away, for example at a hospital or a place where a medical professional practice. The wireless communication between the display device 334 and a further external device is in the embodiment shown in FIGS. 65g and 65h based on WiFi, but could in alternative embodiments be based on for example Bluetooth.

As mentioned, in the embodiment shown in FIGS. 65g and 65h, the wireless communication between the display device 334 and the housing unit 320″ is based on NFC, while the wireless communication between the display device and a further external unit is based on WiFi. As such, the first communication unit of the display device 334 is configured to communicate wirelessly with the housing unit 320″ using a first communication frequency and the second communication unit of the display device 334 is configured to communicate wirelessly with a further external device using a second different communication frequency. For this purpose, the first communication unit of the display device 334 comprises a first antenna configured for NFC-based wireless communication with the housing unit 320″, and the second communication unit comprises a second antenna configured for WiFi-based wireless communication with a further external device. The first and second antennae may be wire-based antennae or substrate-based antennae. As such, the first communication unit is configured to communicate wirelessly with the housing unit 320″ on a first frequency and the second communication unit is configured to communicate wirelessly with the further external device using a second different communication frequency. Also, the first communication unit of the display device 334 is configured to communicate wirelessly with the housing unit 320″ using a first communication protocol (the NFC communication protocol), and the second communication unit is configured to communicate wirelessly with the further external device using a second communication protocol (the WiFi communication protocol). The first and second communication protocols are different which adds an additional layer of security as security structures could be built into the electronics and/or software enabling the transfer from a first to a second communication protocol.

In alternative embodiments, the second communication unit of the display device 334 may be configured to communicate with the further external device by means of, a WLAN type protocol, or a 3G/4G/5G type protocol, or a GSM type protocol.

In the embodiment shown in FIGS. 65g and 65h, the communication range of the first communication unit of the housing unit 320″ is less than a communication range of the second communication unit of the housing unit 320′, such that the communication distance between the housing unit 320″ and the medical implant may be longer than the communication distance between the housing unit 320″ and the display device 334. In the embodiment shown in FIGS. 65g and 65h, the communication range of the first communication unit may be constrained to a length that is less than five times the longest dimension of the minimal bounding box of the display device 334, or more precisely constrained to a length that is less than three times the longest dimension of the minimal bounding box of the display device 334.

In the embodiment shown in FIGS. 65g and 65h, communication between the housing unit 320″ and the display device 334 is only enabled when the housing unit 320″ is connected to the display device 334. I.e. at least one of the housing unit 320″ and the display device 334 is configured to allow communication between the housing unit 320″ and the display device 334 on the basis of the distance between the housing unit 320″ and the display device 334. In the alternative, the housing unit 320″ and/or the display device 334 may comprise a sensor configured to estimate whether the housing unit 320″ is attached to the display device 334 or not, such as a mechanically activated switch or a photo resistive sensor which providing sensor input when the housing unit 320″ and display device 334 are mechanically connected to each other. The signal from the at least one sensor then may be used to permit usage of the communication unit configured for communication with the display device 334.

In the embodiment shown in FIGS. 65g and 65h, communication between the housing unit 320″ and the implantable medical device is only enabled on the basis of a distance between the housing unit 320″ and the implantable medical device. In the embodiment shown in FIGS. 65g and 65h, the distance should be less than twenty times the longest dimension of the minimal bounding box of the display device, or more specifically less than ten times the longest dimension of the minimal bounding box of the display device. The distance between the housing unit 320″ and the medical implant may be measured using electromagnetic waves, or acoustic waves. The process of measuring the distance may comprise triangulation.

In the embodiment shown in FIGS. 65g and 65h, the second communication unit of the display device 334 need to be disabled to enable communication between the display device 334 and the housing unit 320″, and further the second communication unit of the display device 334 needs to be disabled to enable communication between the housing unit 320″ and the medical implant. Also, the second communication unit of the housing unit 320″ needs to be disabled to enable communication between the housing unit 320″ and the medical implant.

In the embodiment shown in FIGS. 65g and 65h, the housing unit 320″ further comprises an encryption unit configured to encrypt communication received from the display device 334 before transmitting the communication to the implanted medical device. The encryption unit may for example be based on one of the following algorithms: AES, Blowfish, DES, Kalyna, Serpent or Twofish. For the purpose for handling the communication, I/O and encryption, the housing unit 320″ comprises a processor which could be a general-purpose microprocessor and/or an instruction set processor and/or related chips sets and/or special purpose microprocessors such as ASICs (Application Specific Integrated Circuit). The processor also comprise memory for storing instruction and/or data.

The housing unit 320″ may comprise a storage unit, such as a battery, for storing energy. The storage unit may be adapted to be charged by the display device 334, or another external device. In some examples, the charging is performed using reverse wireless charging. To that end, the housing unit 320″ may comprise an energy receiver connected to the storage unit, the energy receiver being adapted to wirelessly receive energy from another device. The display device 334 may comprise a primary coil and the housing unit comprise a secondary coil connected to an energy storage of the housing unit, wherein the display device 334 is adapted to wirelessly charge the housing unit using the first coil, and wherein the housing unit is adapted to receive wirelessly transmitted energy through the second coil and store the energy in the storage unit. In one example, the wireless charging may be performed using the Qi standard for wireless charging.

FIGS. 65i and 65j shows an embodiment of the external unit similar to the embodiment described with reference to FIGS. 65g and 65h. The difference being that in the embodiment of FIGS. 65i and 65j, the housing unit 320″ does not clasp the display device 334. Instead, the housing unit comprises two magnets 1510 for magnetically fixating the display device 334 to the housing unit 320″. In alternative embodiments, it is equally conceivable that the external device comprises an intermediate portion, which is fixedly fixated to the housing unit for providing a detachable connection with the display device 334. In the alternative, the intermediate device could be fixedly fixated to the display device 334 and provide a detachable connection with the housing unit 320″.

FIG. 65k shows a system overview of the external device (which could be the external device of the embodiment described with reference to FIGS. 65g and 65h, or of the embodiment described with reference to FIGS. 65i and 65j). The housing unit 320″ is connected to the display device 334. A wireless connection 413 is provided between the housing unit 320″ and the display device 334, and a further wireless connection 413 is provided between the housing unit 320″ and the implanted medical device 100, such that the housing unit can send instructions and updates to the implanted medical device 100, and receive information, parameters (such as sensor values) and alarms from the implanted medical device 100. The communication between the external device and the medical implant 100 is further described in other portions of this disclosure.

In some examples shown in any of FIGS. 65A-65L, the patient remote control or the patient EID may comprise a wireless transceiver for communication with the implant, and a second wireless receiver for communication with a communications network over which the patient remote control or patient EID may communicate with a patient remote control, patient EID, HCP EID or DDI.

In some examples, the patient remote control or the patient EID may be wirelessly charged. Thus, the patient remote control or the patient EID may comprise a first coil for receiving wireless energy to be used or stored at the patient remote control or the patient EID.

### Dual Remote Controls

FIG. 65L shows one embodiment of a communication system for transmission of data to and/or from an implantable medical device 100. The communication system comprises an implantable medical implant, a first remote control 320″, and a second remote control 320″″. The first remote control 320″ (also referred to as patient external device 320″) is operable by a user and comprises a first wireless communication unit configured for wireless transmission of data to and/or from the implantable medical device 100. The second remote control 320″″ is inoperable by a user (i.e. may not comprise any input means such as buttons, switches, or user interface to receive any input directly from a user) and comprises a second wireless communication unit configured for wireless transmission of control commands and/or data to and/or from the implantable medical device 100. The second remote control 320″″ further comprises a third communication unit for communicating with a patient display device 334 (e.g. a smartphone, smartwatch, tablet, and/or the like).

By having two separate remote controls, the security of the implant may be improved, as there are two separate ways of controlling the implant. Thus, in case of a malfunction of either of the remote controls, the implant may still be controlled. Furthermore, this allows for the second remote control to be smaller or more compact since it is inoperable by the user other than through a patient display device or another external device. The second remote control may thus be smaller and potentially less expensive.

The first and second remote controls 320″, 320″″ each comprise a wireless transceiver 328 for communicating with the implantable medical device 100. The first and/or second remote control 320″, 320″″ is capable of controlling the operation of the implantable medical device 100 via the controller 300 (for controlling the implantable medical device and for communicating with devices external to the body of the patient and/or implantable sensors). The first and/or second remote control 320″, 320″″ may control the operation of the implantable medical device 100 by controlling pre-set functions of the implantable medical device 100, e.g. for operating an active portion of the implantable medical device 100 for performing the intended function of the implantable medical device 100.

The first and/or second remote control 320″, 320″″ is able to communicate with implantable medical device 100 using any standard or proprietary protocol designed for the purpose. At least one of the first remote control 320″, the second remote control 320″″, and the implantable medical device 100 may, e.g., comprise a Bluetooth (BT) transceiver. In particular, the wireless transceiver 328 may comprise a BT transceiver, and the and/or second remote control 320″, 320″″ may be configured to communicate with implantable medical device 100 using BT. In one embodiment, the first and/or second remote control 320″, 320″″ is configured to communicate with implantable medical device 100 using NFMI.

In an alternative configuration, the first and/or second remote control 320″, 320″″ may communicate with the implantable medical device 100 using a combination of Ultra-Wide Band (UWB) wireless communication, NFMI and/or BT. For example, at least one of first remote control 320″, the second remote control 320″″, and the implantable medical device 100 may comprise a UWB transceiver. The use of UWB technology enables positioning of the first and/or second remote control 320″, 320″″ which can be used by the implantable medical device 100 as a way to establish that the first and/or second remote control 320″, 320″″ is at a position which the implantable medical device 100 and/or the patient can acknowledge as being correct, e.g. in the direct proximity to the medical device 100 and/or the patient, such as within reach of the patient and/or within 1 or 2 meters of the implantable medical device 100.

When a combination of BT and UWB and/or NFMI technology is used, the UWB or NFMI technology may be used for location-based authentication of the first and/or second remote control 320″, 320″″, whereas the communication and/or data transfer could take place using BT or any other way of communicating different from the UWB or NFMI. The UWB or NFMI signal could in some embodiments also be used as a wake-up signal for the controller 300, or for the BT transceiver, such that the BT transceiver in the implantable medical device 100 can be turned off when not in use, which eliminates the risk that the BT is intercepted, or that the controller 300 of the implantable medical device 100 is hacked by means of BT communication. In embodiments in which a BT (or alternatives)/UWB combination is used, the UWB connection may be used also for the transmission of data. In the alternative, the UWB connection could be used for the transmission of some portions of the data, such as sensitive portions of the data, or for the transmission of keys for the unlocking of encrypted communication sent over BT.

The first remote control 320″ may be configured to control functions of the implantable medical device 100 based on user input to the first remote control 320″. In particular, the first remote control 320″ may comprise an input device for receiving a first user input, wherein the first remote control 320″ is configured to transmit the first user input to the implantable medical device 100. The first remote control 320″ may comprise a computing unit 326 which runs a software application for communicating with the implantable medical device 100. The computing unit 326 may receive the first user input directly from control buttons 335 arranged on the first remote control 320″. The computing unit 326 may be configured to encrypt control commands before transmission to the implantable medical device 100. The computing unit 326 is further configured to transform the received first user input into control commands for wireless transmission to the implantable medical device 100.

The second remote control 320″″ may comprise a wireless transmitter 325 configured for transferring energy wirelessly. The energy may be in the form of a magnetic field or any other signal such as electromagnetic, radio, light, sound or any other type of signal to transfer energy wirelessly to a wireless receiver 395 of the implantable medical device 100. The wireless receiver 395 of the implantable medical device 100 is configured to receive the energy in the form of the magnetic field and transform the energy into electric energy for storage in an implantable energy storage unit 40 of the implantable medical device 100, and/or for consumption in an energy consuming part of the implantable medical device 100 (such as the operation device, controller 300 etc.). In other words, the implantable energy storage unit 40 may be adapted to be wirelessly charged. The first remote control may similarly comprise a wireless transmitter for transferring energy wirelessly to the implantable medical implant. The implantable energy storage unit 40 may particularly be connected to the wireless receiver 395 for receiving wireless energy from the first and/or second remote control 320″, 320″″.

In the embodiment shown in FIG. 65L, the second remote control 320″″ is configured to communicate with a patient display device 334. Here, the patient display device 334 comprises the consumer electronics device. In particular, the second remote control 320″″ is configured to receive a second user input from the patient display device 334 and to transmit the second user input to the implantable medical implant. The second remote control 320″″ may receive the second user input from a control interface 334i displayed on the patient display device 334 operated by the patient.

The patient display device 334 may for example be a mobile phone, a tablet or a smart watch. The display device 334 may, for example, communicate with the second remote control 320″″ by means of BT, but any wireless or wired communication means may be used. The control interface 334i, e.g. in the form of a web-view portal, may be transmitted from the second remote control 320″″ to the patient display device 334 over BT. Control commands in the form of inputs from the patient to the control interface 334i are transmitted from the patient display device 334 to the second remote control 320″″, providing input to the second remote control 320″″ equivalent to the input that may be provided using the control buttons 335 or other input means of the first remote control 320″. The control commands created in the patient display device 334 may be encrypted in the patient display device 334 and transmitted to the second remote control 320″″ using BT or any other communication protocol.

The second remote control 320″″ may be implemented and/or integrated in an accessory to the patient display device 334. The second remote control 320″″ may, e.g., form part of a mobile phone case (i.e. smartphone case) for a mobile phone. Alternatively, the second remote control 320″″ may be integrated in a case for a personal computer, or a body worn camera, or any other suitable type of external device as described herein. The case may for example be connected to the patient display device 334 (e.g. mobile phone) using a wire from the case and connected to the patient display device (e.g. a charging port).

The second remote control 320″″ may not be connected to the DDI or the Internet, thereby increasing security. The second remote control 320″″ may have a private key, in particular the second remote control 320″″ may be activated by a private key 333′ of the patient for a certain time period. This may activate the function of the patient display device 334 and the remote wed-view display portal supplied by the second remote control to the patient display device 334.

The patient's private key 333′ may be supplied in a patient private key device comprising a smartcard that may be inserted or provided close to the first remote control 320″ and/or close to the second remote control 320″″ to activate a permission to communicate with the implantable medical device 100 for a certain time period. The patient's private key 333′ is in the embodiment shown in FIG. 65L in the form of a key card having an interface for communicating with the first remote control 320″, the second remote control 320″″, and/or another device or control. The NFC-transmitter 339 and/or the printed QR-code 344 may be used as means for accessing the control interface 334i of the display device 334. In addition, the display device 334 may require a pin-code and/or a biometric input, such as face recognition or fingerprint recognition, for controlling the implantable medical implant.

The patient display device 334 may comprise auxiliary radio transmitters for providing auxiliary radio connection, such as Wi-Fi or mobile connectivity (e.g. according to the 3G, 4G or 5G standards). The auxiliary radio connection(s) may have to be disconnected to enable communication with the second remote control 320″″. Disconnecting the auxiliary radio connections reduces the risk that the integrity of the control interface 334i displayed on the patient display device 334 is compromised, or that the control interface 334i displayed on the patient's display device 334 is remote controlled by an unauthorized device.

The data transmitted in the communication system may comprises a control command for the medical implant. Hence, real-time, remote management of patient care is provided and settings of the medical implant may be adjusted, e.g., based on the patient's current health status. Thus, invasive procedures may be averted while efficiency of healthcare delivery and patient comfort may be improved. Furthermore, more responsive and/or personalized healthcare may be provided, as adjustments can be made promptly in response to changes in the patient's condition.

At least one of the first wireless communication unit of the first remote control 320″ and the second wireless communication unit of the second remote control 320″″ may be configured to send and/or receive data using near-field magnetic induction (NFMI). Thus, enhanced security and reliability of the communication system may be provided. NFMI creates a private, secure communication link that is difficult to intercept or disrupt due to the magnetic field being spatially confined and thus less susceptible to interference compared to traditional radio frequency communication. Furthermore, NFMI penetrate materials such as water and body tissue, making it particularly suitable for communication with medical implants.

Further, at least one of the first wireless communication unit and the second wireless communication unit may comprise a transmitter coil for modulating a magnetic field for transmitting the data. In turn, the implantable medical implant may comprise a receiving coil and an NFMI receiver connected to the receiving coil to receive the data. The transmitter coil(s), in conjunction with the receiving coil and NFMI receiver of the implantable medical implant, may provide efficient and reliable data transfer. The use of a magnetic field for data transmission, which is typically more energy-efficient than traditional radio frequency communication, may additionally reduce power consumption and thereby extend an operational period of the implantable medical implant.

The transmitter coil(s) may be configured to modulate a magnetic field, and the NFMI receiver may be adapted to measure the magnetic field in the receiving coil. A modulated magnetic field may enable the construction of specific signal patterns for the data transmission such that transmission of complex data sets is enabled.

At least one of the first wireless communication unit and the second wireless communication unit may further be configured to wirelessly charge the implantable medical implant using NFMI. In particular, at least one of the first wireless communication unit and the second wireless communication unit may be, and/or act as, the wireless transmitter 325 configured for transferring energy wirelessly

Similarly, the implantable medical implant may comprise a coil for receiving wireless energy for charging the implant via NFMI. The coil of the implantable medical implant may, e.g., form part of, or be, the wireless receiver 395.

The second and third communication units of the second control unit 320″″ may be configured to transmit and/or receive data using different network protocols. In other words, the second and third communication units may be designed to send and/or receive data using separate and/or alternate networking standards. Thus, the communication system can communicate across a variety of network environments and conditions. A multi-protocol support may enhance interoperability of the second remote control 320″″, allowing for communicate with a wide range of devices and systems (such as the patient display device 334 and the implantable medical device 100). Alternatively, or additionally, the second and third communication units may for the same reasons be configured to transmit and/or receive data using different frequency bands.

The standard, communication, and/or network protocols discussed herein may be any one or more from the list of: Radio Frequency type protocol, RFID type protocol, WLAN type protocol, Bluetooth type protocol, BLE type protocol, NFC type protocol, 3G/4G/5G type protocol, and GSM type protocol.

In an example, the second communication unit has a longer effective range than the third communication unit. In other words, the second communication unit may be able to communicate with a device (e.g., the implantable medical device 100) from a further distance than the distance at which the third communication unit is able to communicate with another device (e.g., the patient display device 334). For example, the second communication unit may use a network protocol with a longer effective range than the network protocol of the third communication unit.

In the specific embodiment disclosed in FIG. 65L, the wireless connections between the different units are as follows. The wireless connection 413 between the patient display device 334 and the second remote control 320″″ is based on BT or any other communication protocol disclosed herein. The wireless connection 414 between the second remote control 320″″ and the implantable medical device 100 is based on BT and UWB or any other communication protocol disclosed herein. The wireless connection 416 between the first remote control 320″ and the implantable medical device 100 is based on BT, UWB, and the charging signal, or any other communication or energizing pathway disclosed herein.

The wireless connections specifically described in the embodiment shown in FIG. 65L may be replaced or assisted by wireless connections based on radio frequency identification (RFID), near field communication (NFC), Bluetooth, Bluetooth low energy (BLE), or wireless local area network (WLAN).

The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves. An embodiment in which the second remote control 320″″ is comprised in a housing is shown in FIG. 65LL.

### Voice Control

FIG. 65M schematically shows a medical implant 10 when implanted in a patient 1. The medical implant comprises a processor 1300 connected to a medical device 100, which may be comprised in the implant 100. The medical device may be any medical device or implant discussed herein, and may be configured to control or monitor a function of the body of a patient. The medical implant 10 may further comprise or be connected to a microphone 1369. The microphone 1369 may receive audio and transfer that audio to the processor 1300. The processor 1300 may thus receive audio via microphone 369.

The processor 1300 may have two modes of operation, a learning mode for learning voice commands and an operational mode for recognizing and transmitting voice commands to the medical device 110 or the medical implant 1100. The processor 1300 may be configured to, when in the learning mode, receive a first audio training phrase and creating a transfer function, the transfer function being based on the first audio training phrase, wherein the transfer function is configured to adjust the amplitude of at least one frequency of audio received at the medical device 110 for enhancing audio received at the medical implant 100 to facilitate detection of voice commands. To this end, the processor 1300 may comprise a transfer function unit 1370. The processor 1300 may be further adapted to receive a second audio training phrase, the second audio training phrase comprising a voice command, wherein the voice command comprises an instruction for the control of the medical implant 1100 and/or the medical device 110. The processor 300 may be further configured to use the transfer function for generating an enhanced second audio training phrase in the medical implant, and associating the enhanced second audio training phrase with the instruction for the control of the medical implant. Thus, the medical implant 110 has learned that the voice command comprised in the enhanced audio training phrase corresponds to the instruction.

In some examples, the audio training phrases are inputted into a transfer function unit 1370 for creating the transfer function. The processor may further comprise a learning unit 1371 for associating the enhanced second audio training phrase with the instruction for the control of the medical implant. The learning unit 1371 may, for example, comprise an algorithm based on machine learning for learning to associate the enhanced audio training phrase with the correct instruction for the medical device 1100. The voice commands, the instructions and any association between the voice commands may be stored in a memory unit 1373 comprised in or connected to the processor 1300.

The processor 1300 may be further adapted to receive audio input, process the audio input in order to determine an instruction and to transfer that instruction to the medical device 1100. In order to determine the instruction, the processor may use the transfer function 1370 to enhance the audio input and then determine the instruction associated with the enhanced audio input (as associated by the method described herein). The instruction may also be called a control command or a command. The instruction may be determined by and/or be transferred to the medical device 1100 via a command unit 1372 comprised in or connected to the processor 1300. The instruction may relate to a function of the medical device 1100 and may cause the medical device 1100 to perform an action, or it may relate to any other function of the medical implant 10, such as the processor 1300.

By learning voice commands, it may be meant that the processor associates an audio input with a control command for the medical device.

The processor 1300 may be further configured to, when in the operational mode, receive an audio command phrase for the medical device 1100 or implant 110. The processor 1300 may be further configured to apply a transfer function to create an enhanced audio command phrase. The transfer function may have been created as discussed above. The processor 1300 may determine a corresponding command for the medical based on the enhance audio command phrase, and send the command to the medical device 1100 or the medical implant 110. The medical device 1100 or implant 110 may then execute the command.

When the medical implant is implanted in the body, typically the medical implant stays in the same place in the body. Thus, it has been realized that any noise or distortion created by the body to audio commands may be substantially the same. By creating a transfer function based on a first audio training phase when the medical implant is implanted in the body, any noise created by the body or any distortions to the audio training phase caused by the body itself can be accounted for. The method thus accounts for that that the noise and distortions created by the body is substantially the same over time. Thus, the transfer function may account for those disturbances when enhancing any audio received by the medical implant. In this way, audio received by the medical implant may be enhanced, i.e., any known disturbances created by the body to the audio may be accounted for, before the medical implant does any further processing. Since the audio is enhanced before any training or processing, the process of recognizing which command for the medical implant the audio relates to may be simplified. That is, the processing power needed for recognizing voice commands may be reduced, which is advantageous in medical implants since the size of the implant may be decreased.

FIG. 65n shows a flow chart for a method for training a medical implant to recognize a voice command, according to some embodiments. The method 200 comprises receiving 210, by a medical implant, a first audio training phrase, when the medical implant is implanted in the body of the patient.

The method further comprises creating 220 a transfer function, the transfer function being based on the first audio training phrase, wherein the transfer function is configured to adjust the amplitude of at least one frequency of audio received at the medical device for enhancing audio received at the medical implant to facilitate detection of voice commands. Creating 220 a transfer function based on a first audio input phase when the implant has been implanted in a patient allows for specifically correcting the audio input phrase for noise and/or distortion caused by the patient's body specifically.

The creation of the transfer function may be based on training a machine learning model.

A purpose of the transfer function may be to adjust the audio input for distortions or noise specific to the body the implant has been implanted into. After the audio input has been adjusted, or enhanced, the audio input may be in a better condition for use in later steps of the method, such as for recognizing a command for the medical implant comprised in the audio input. In that way, there may be a two-step method for training the medical implant to recognize commands. Since the audio input has been adjusted or enhanced, the voice recognition of the command in the audio input may be easier, which may allow for using less processing power.

In some embodiments, the creating 220 a transfer function may further comprise to compare 221 the first audio training phrase with a stored audio phrase to determine a difference between them. Based on the difference, the transfer function may be created 222. In other words, the method may comprise creating a transfer function based on a difference between a stored audio phrase and the first audio phrase.

As an illustrative example only, the stored audio phrase may comprise a specific command or test phrase. When in a training session, a user of the implant or another person that the implant should be trained for, may speak the same specific command or test phrase. The command or test phrase may then be captured by the microphone of the implant, and transferred to the learning unit of the processor. The learning unit may then compare the received command or test phrase with the stored command or test phrase, and then, based on the difference(s), create a transfer function which takes the differences into account. The differences between the received command or test phrase and the stored command or test phrase may be indicative of a noise or distortion created by the body in which the implant has been implanted.

The method 200 may further comprise inputting 230 a second audio training phrase to the medical implant, the second audio training phrase comprising the voice command, the voice command comprising an instruction for the control of the medical implant.

The second audio training phrase may be used as input to the transfer function in order to create an enhanced audio training phrase. In this way, any noise or distortion created by the body may be alleviated by the transfer function, thus resulting in an enhanced audio training phrase. Thus, the method may further comprise using 240 the transfer function for generating an enhanced second audio training phrase in the medical implant.

The enhanced audio training phrase may then be associated 250 with the instruction for the control of the medical implant. That is, the method may comprise training a command unit to associate the second audio training phrase to a command for the medical device. The training may comprise training a machine learning model to associate enhanced audio training phrases with commands for the medical implant.

By first creating a transfer function, any following audio input may be enhanced by using the transfer function, and thus the associating of a second audio training phrase with a command may be simpler, i.e. less computationally intense, as the quality of the enhanced audio may be better that the originally audio received by the microphone of the implant. The method also allows for avoiding training the medical implant on distorted audio or audio with a lot of noise, thus improving the quality of the training.

FIG. 65o shows a flow chart for a method 300 for using voice commands to control a medical implant, according to some embodiments. In some examples, the voice commands have been learnt with the method described with reference to FIGS. 65m and 65n.

The method 300 comprises receiving 310 an audio command phrase for the medical device. The method further comprises applying 320 a transfer function to create an enhanced audio command phrase.

The method may further comprise to determine 320 a corresponding command for the medical based on the enhance audio command phrase, and send 340 the command to the medical device. The medical device may then execute 350 the command. By running the audio command phrase through the transfer function, the audio quality of the audio command phrase may be improved, thus allowing for an easier recognition of the corresponding command. This may make the recognition or determination of the command for the medical device less computationally intensive.

Controlling energy transfer at the implant

Any of the implantable medical implants described herein are configured to wirelessly receive energy for powering or charging the implantable medical implant. When transferring energy to an implantable medical implant it is important to adequately control the energy transfer. If the energy transferred or received at the medical implant is excessive, it may harm the patient. For example, if the position of external device relative to the receiving unit changes during energy transfer, the energy transferred may also increase or decrease drastically. This situation could cause severe problems since the implant cannot “consume” the suddenly very high amount of supplied energy. Unused excessive energy must be absorbed in some way, resulting in the generation of heat, which is highly undesirable as it may harm the patient. Hence, if excessive energy is transferred from external device the receiving unit, the temperature of the implant will increase, which may damage the surrounding tissue or otherwise have a negative effect on body functions. It is therefore highly desirable to always supply the right amount of energy to an implanted medical device during operation. Similarly, if too much energy is received by the implant, there may be temperature increases which may harm the patient. It has thus been realized that controlling the energy transfer at the medical implant may be advantageous.

An embodiment of a system for transferring energy to an implantable medical device will now be described with reference to FIGS. 65A-C. Alternatively, “transferring energy” may be referred to as “charging”. A corresponding a method for wireless energy transfer from an external energy source located outside the patient to an internal energy receiver 305 located inside the patient is also provided.

The system comprises an external energy source, or a charger, and an internal energy receiver 305. The external energy source may be comprised in any of the external devices, i.e. devices arranged outside of the body of a patient, described herein. The internal energy receiver 305 is connected to an implantable medical device 300 for supplying received energy thereto. Internal energy receiver 305 may be configured to determine an accumulated amount of received energy; determine a current change in the received energy, determine a control signal reflecting the accumulated received energy and the change in the received energy, and controlling the energy transfer based on the control signal. As an alternative, the determination of the control signal may be omitted, and the controlling may be performed based on the accumulated amount of energy and the current change.

By “controlling the energy transfer” it may be meant or include adjusting the energy transfer efficiency, controlling switches affecting the energy transfer, controlling a part of the internal energy receiver, controlling a part of the external energy source, turning the energy transfer off completely, or any other way of affecting the energy transfer.

In one embodiment the external energy source or the internal energy receiver 305 may comprise an energy transfer controller for controlling the energy transfer. The energy transfer controller may be configured to determine the rate of change of the received energy and/or the accumulated amount of received energy, and adjust the energy transfer based on the determined parameters.

Advantageously, the energy transfer may be controlled or adjusted by the internal energy receiver 305, as the internal energy receiver 305 is capable of directly determining how much energy is received in the internal energy receiver and faster determine if there is a risk to the patient or the medical implant. Thus, the internal energy receiver 305 may be configured to determine an accumulated amount of transferred energy is determined by the internal energy receiver 305. The internal energy receiver 305 may alternatively or in combination, be configured to determine a current change in the energy transfer. Further, the internal energy receiver 305 may be configured to determine a control signal for controlling the energy transfer. The control signal may be used in the internal energy receiver 305 for adjusting the receiving of energy, or it may be transmitted to the external energy source, and the external energy source may be configured to adjust the transmitted energy based on the control signal. That is, the controlling of the energy transfer may be performed by the internal energy receiver 305.

In some examples, the controlling of the energy transfer may be performed by the external energy source.

In some examples, the internal energy receiver 305 is configured to measure, via a measuring unit, an accumulated energy received a period of time and/or to measure a current change in energy received, and to control the energy received based on the accumulated energy and/or the current change. In some examples, this may be performed using a PID regulator, which will be described in the following.

In some examples, the controller comprised in the internal energy receiver comprises a PID regulator. Such a PID regulator may be used to control the difference between a received voltage and a desired voltage level. The PID regulator may control a switch to signal to selectively de-tune the receiving coil of the internal energy receiver. Alternatively, or in combination, the PID may regulate the switch to modulate the power signal. The PID regulator may respond quickly to changes in the power levels and provides increased control over the pulse width modulation of the power signal.

A PID regulator may be used for controlling any energy transfer as discussed herein.

### Pulse Width Modulation (PWM)

In some embodiments, the energy is supplied from the primary coil to the secondary coil using energy pulses. The pulses are achieved using modulation techniques. For example, modulation (PWMT—Pulse width modulation technique) of the pulses may be created with a system that controls the power using a continuous square wave pulse signal with a constant frequency where the duty cycle of the pulses is varied or a system that controls power using a continuous square wave pulse train signal with both constant frequency and constant pulse with and thereby adjusting the duty cycle width of the train of pulses. The PWMT may be used to digitally vary the amount of power from the power amplifier that drives the transmitting coil. Thus, the amount of energy transferred from the primary coil to the secondary coil may be controlled.

In some examples, the energy is supplied using a pulse pattern. In those examples, the receiving unit 305 may be configured to receive transcutaneously transferred energy in pulses according to a pulse pattern, and the measurement unit may be configured to measure a parameter related to the pulse pattern. In some examples, the controller is configured to control the energy received (for example by a variable impedance or via switches as described below) in response to the pulse pattern deviating from a predefined pulse pattern.

In some examples, the energy transmitted may be varied by varying the width of the energy pulses and having constant frequency and constant amplitude. The pulse width is achieved with a modulation technique, (hereafter PWMT) (in the preferred embodiment many times per second), to control the amount of energy transferred from the external energy transmitting coil in the system to the implanted receiver. The PWMT is used to digitally vary the amount of power from a power amplifier that drives the transmitting coil. There are several different ways to achieve the PWMT to control the amount of output energy from the power amplifier to the transmitting coil. Generally, modulation of the pulse width may be created with a system that controls the power using a continuous square wave pulse signal with a constant frequency where the duty cycle of the pulses are varied or a system that controls power using a continuous square wave pulse train signal with both constant frequency and a constant pulse width and thereby adjusting the duty cycle width of the train of pulses. These two basic techniques as well as most modifications of them can be used to control the output power of the transmitting coil.

The transmission of wireless energy from the external energy transmitting device may be controlled by applying to the external energy transmitting device electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

Advantageously, the PWM embodiments described herein may be combined with any embodiment relating to controlling energy transfer to an implantable medica device, variable impedance, resonant circuit, NFMI, large coil, or any other implantable medical device being in any way configured to receive energy wirelessly, as described herein.

Variable impedance

According to one embodiment described with reference to FIG. 65A-65C, the controller 300 of the implantable system 10 comprises a receiving unit 305 or energy receiver 305 comprising a coil 192 (specifically shown in FIG. 65B′) configured for receiving transcutaneously transferred energy. The receiving unit 305 further comprises a measurement unit 194 configured to measure a parameter related to the energy received by the coil 192 and a variable impedance 193 electrically connected to the coil 192. The receiving unit 305 further comprises a switch 195a placed between the variable impedance 193 and the coil 192 for switching off the electrical connection between the variable impedance 193 and the coil 192. The controller 300 is configured to control the variable impedance 193 for varying the impedance and thereby tune the coil 192 based on the measured parameter. The controller 300 is further configured to control the switch 195a for switching off the electrical connection between the variable impedance 193 and the coil 192 in response to the measured parameter exceeding a threshold value. As such, the coil can be tuned or switched off to reduce the amount of received energy if the amount of received energy becomes excessive.

The controller 300 may further be configured to vary the variable impedance in response to the measured parameter exceeding a threshold value. By varying the variable impedance, the tuning of the coil may be varied, thus affecting the resonant frequency of the receiving coil. In this way, the efficiency of the reception of energy may be varied.

The measurement unit 194 is configured to measure a parameter related to the energy received by the coil 192 over a time period and/or measure a parameter related to a change in energy received by the coil 192 by for example measure the derivative of the received energy over time. The variable impedance 193 is in the embodiment shown in FIG. 65B′ placed in series with the coil 192. In alternative embodiments it is however conceivable that the variable impedance is placed parallel to the coil 192.

The first switch 195a is placed at a first end portion 192a of the coil 192, and the receiving unit 305 further comprises a second switch 195b placed at a second end portion of the coil 192, such that the coil 192 can be completely disconnected from other portions of the implantable system 10. The receiving unit 305 is configured to receive transcutaneously transferred energy in pulses according to a pulse pattern. The measurement unit 194 is in the embodiment shown in FIG. 65B′ configured to measure a parameter related to the pulse pattern. The controller 300 is configured to control the variable impedance in response to the pulse pattern deviating from a predefined pulse pattern. The controller 300 is configured to control the switch 195a for switching off the electrical connection between the variable impedance 193 and the coil 192 in response to the pulse pattern deviating from a predefined pulse pattern. The measurement unit is configured to measure a temperature in the implantable system 10 or in the body of the patient, and the controller 300 is configured to control the first and second switch 195a,195b in response to the measured temperature.

The variable impedance 193 may comprise a resistor and a capacitor and/or a resistor and an inductor and/or an inductor and a capacitor. The variable impedance 193 may comprise a digitally tuned capacitor or a digital potentiometer. The variable impedance 193 may comprise a variable inductor. The first and second switch comprises a semiconductor, such as a MOSFET. The variation of the impedance is configured to lower the active power that is received by the receiving unit. As can be seen in FIG. 65B′, the variable impedance 193, the first and second switch 195a,195b and the measurement unit 194 are connected to the communication unit/controller 300 and the receiving unit 305 is connected to an energy storage unit 40 such that the energy storage unit 40 can store energy received by the receiving unit 305.

Resonant circuit

FIG. 65P schematically illustrates a system 500 comprising an external unit 510 and an implantable medical device 505. The external unit 510 is adapted to be arranged outside of a body. The implantable medical device 505 is adapted to be implanted into a body of a patient. The external unit 510 comprises a transmitting circuit 512, a transmitting coil 514 and electrical connections 502. The electrical connections 502 electrically connect the transmitting circuit 512 with the transmitting coil 514. The electrical connections 502 may, for example, be wires or any electrically conducting material, or they may be wireless electrical connections. The electrical connections 502 may have intrinsic electrical resistance.

The implantable medical device 505 comprises an energy consuming part 528. The implantable medical device 505 further comprises receiving units 530 for receiving transcutaneously transferred energy, wherein the receiving unit 530 is configured to transfer the received energy to the energy consuming part 528. In FIG. 65P, three receiving units 530 are illustrated. However, the medical device 505 may comprise any number of receiving units 530. The medical device 505 preferably comprises two or more receiving units 530. Each receiving unit 530 comprises a receiving circuit 522, an impedance unit 526 and a receiving coil, or coil, 524, and electrical connections 502. The electrical connections 502 in the receiving unit 530 electrically connect the receiving circuit 522 to the impedance unit 526 and to the coil 524. Electrical connections 502 electrically connect the receiving unit 530 to the energy consuming part 528. The receiving unit 530 may be directly connected to the energy consuming part 528. The receiving unit 530 may be connected to intermediate circuits, wherein the intermediate circuits are connected to the energy consuming part 528. The intermediate circuits may process the output from the receiving unit 530 and prepare it for the energy consuming part 528. The intermediate circuit may comprise amplifiers, switches, filtering, modulators, other signal transformers, or a combination thereof. In FIG. 65P, the impedance units 526 are connected in parallel to the coil 524. The impedance units 526 and the respective coil 524 may instead be connected in series, partially in series and partially in parallel, or in any other way. I FIG. 65P, the impedance units 526 are capacitors. The impedance unit 526 may consist of inductors, capacitors, capacitors and resistances, inductors and resistances, or a mixture thereof. The impedance unit 526 may have a variable impedance.

The implantable medical device may further comprise a measurement unit 521 and a controller 520. The measurement unit 521 may be configured to measure a parameter related to energy transfer from the external unit 510 to the implantable medical device 505. The controller 520 may be configured to control the subcutaneously received energy to the energy consuming part 528. The controller 520 may be configured to control the subcutaneously received energy based on the parameter measured by the measurement unit 521. The controller 520 may control the impedance units 526. The controller 520 may control a variable impedance of the impedance unit 526.

The implantable medical device 505 and the external unit 510 are electrically coupled.

The transmitting circuit 512 generates an alternating current in the transmitting coil 514. The alternating current of the transmitting coil 514 induces a current in the coil(s) 524. The receiving unit 530 is configured to receive transcutaneously transferred energy from the external unit 505 via the coil 524. One external unit 510 may transfer energy to many receiving units 530 having a respective coil 524.

The inductance of the coil 524 and the impedance of the corresponding impedance unit 526 contributes to a resonance frequency of the receiving unit 530. The inductance of the coils 524 and/or the impedance of the corresponding impedance unit 526 may differ in size between the respective receiving units 530. This may cause receiving units 530 to have different resonance frequencies in relation to each other. A variable impedance of the impedance unit 526 may allow the resonance frequencies of the receiving unit 530 to be tuned. The controller 520 may be able to tune the resonance frequency of each of the receiving units 530 individually by controlling the respective impedance unit 526. The receiving unit 530 may transfer different amounts of energy to the energy consuming device 528 depending on the frequency of an alternating magnetic field generated by the external device 510 and the resonance frequency of the receiving unit 530. By having different resonance frequencies for receiving units 530, a better energy transfer efficiency of the implantable medical device 505 may be obtained. Each receiving unit 530 may be designed to, or be fined tuned to, have the resonance frequency adapted to different frequencies of the external unit 510. By having different resonance frequencies of the receiving units 530, different external units 510 may be used, which is illustrated in FIG. 65Q.

FIG. 65Q schematically illustrates a system 540 comprising a second external unit 511 and an implantable medical device 545. The second external unit 511 comprises several transmitting units 509, wherein each transmitting unit 509 is similar to the external unit 510 illustrated in FIG. 65P, described above. The implantable medical device 545 illustrated in FIG. 65Q comprises an energy consuming part 528 and receiving units 530. The energy consuming part 528 of FIG. 65Q is similar to the energy consuming part 528 of FIG. 65P, described above. The receiving units 530 of FIG. 65Q are similar to the receiving units 530 of FIG. 65P, described above. The implantable medical device 545 of FIG. 65Q may further comprise a measurement unit and/or a controller similar to the measurement unit 521 and the controller 520 of FIG. 65P, described above.

FIG. 65Q illustrates an implantable medical device 545 comprising two receiving units 530. As mentioned above, there may be any number of receiving units 530 in the implantable medical device 545. As mentioned above, the receiving units 530 are connected to the energy consuming part 528, directly or via an intermediate circuit. The receiving units 530 are adapted to receive transcutaneously transferred energy from the external device 511. The external device 511 is adapted to transmit energy to the receiving unit 530 via the transmitting units 509 in the external device 511.

The external unit 511 of FIG. 65Q illustrates several transmitting units 509. The transmitting units 509 may all be comprised in one device, in separate devices, or a combination thereof. Each transmitting unit 509 may be adapted to send energy on a separate frequency. The transmitting units 509 illustrated in FIG. 65Q may be configured to transmit different frequencies. A transmitting unit 509 may be comprised in any external device or remote control described herein, a charging device, such as a smartphone, a qi charger, wireless charging pad, any device comprising a coil configured to send out energy, or any device configured to produce an oscillating a magnetic field. Each receiving unit 530 may be adapted to receive energy from a different transmitting unit 509 by having its resonance frequency configured to match the frequency of the transmitting unit 509. The transmitting units 509 could transmit sequentially, simultaneously, or have one or more transmissions partially overlap.

An advantage of multiple transmitting units 509 is that a better energy transfer efficiency of the implantable medical device 545 may be obtained. Each receiving unit 530 may be tuned to receive energy of a specific frequency of a corresponding transmitting unit 509, so that the receiving units 530 could be charged by their respective transmitting unit 509. Each receiving unit 530 may receive a respective transmitted energy sequentially, simultaneously, and/or independently of the other receiving units 530. An advantageous transmitted energy for a receiving unit 530 may be energy with the frequency of the resonance frequency of the respective receiving unit 530, energy with a frequency within a symmetric or nonsymmetric range around the resonance frequency of the respective receiving unit 530, or energy with a frequency that is at an offset from the resonance frequency of the respective receiving unit 530.

Each receiving unit 530 comprises a coil 524 and a resonance frequency. The resonance frequency is a function of the coil 524. Instead of a coil 524 and one resonance frequency, a part of a coil 524 may contribute to a resonance frequency, meaning that a coil 524 may have several resonance frequencies. This is illustrated in FIG. 65R.

FIG. 65R schematically illustrates an implantable medical device 565. The implantable medical device 565 is adapted to be implanted into a body of a patient. The implantable medical device 565 comprises an energy consuming part 528, similar to the energy consuming parts of FIGS. 65R and 65Q, described above. The implantable medical device 565 of FIG. 65R may further comprise a measurement unit and/or a controller similar to the measurement unit 521 and the controller 520 of FIG. 65P, described above. The medical device 565 may be configured to receive energy from an external unit, such as the external unit 510 of FIG. 65P and/or the external unit 511 of FIG. 65Q.

The implantable medical device 565 further comprises a receiving unit 535 for receiving transcutaneously transferred energy, wherein the receiving unit 535 is configured to transfer the received energy to the energy consuming part 528. The receiving unit 535 comprises a receiving circuit 523. The receiving unit 535 comprises a receiver coil, wherein the receiver coil comprises a coil with one or more center taps, a multitude of coils in parallel, or a combination thereof. Center taps do not have to be positioned in the center of a coil. Parts of a receiver coil are coil portions 525. The receiving unit 535 of FIG. 65R further comprises impedance units 526, similar to the impedance units 526 of FIG. 65P, described above. The receiving unit 535 of FIG. 65R further comprises electrical connections 502, similar to the electrical connections 502 of FIG. 65P, described above. The electrical connections 502 connect the receiving unit 535 to the energy consuming part 528. As mentioned for the receiving unit 530 of FIG. 65P, the receiving unit 535 of FIG. 65R may be connected directly to the energy consuming part 528 or connected first to intermediate circuits, wherein the intermediate circuits are connected to the energy consuming part 528. Intermediate circuits may be the same as described for FIG. 65P above.

The electrical connections 502 in the receiving unit 535 connect the receiving circuit 523 to the impedance units 526 and the coil portions 525 so that each impedance unit 526 is connected to a respective coil portion 525. The impedance unit 526 and the respective coil portions 525 form a receiving portion. The receiving portions may be seen as akin to the receiving units 530 of FIG. 65P, described above. FIG. 65R illustrates each impedance unit 526 being connected in parallel to the respective coil portion 525. The impedance unit 526 may be connected in series, or partially in series and partially in parallel, with the respective coil portion 525. The receiving circuit 523 is connected in parallel to the impedance unit 526. The receiving circuit 523 may be connected in parallel to a portion of the impedance unit 526. The receiving circuit 523 may be connected in series with the entire, or a portion of the, impedance unit 526.

The inductance of the coil portion 525 and the impedance of the corresponding impedance unit 526 contribute to a resonance frequency of the corresponding receiving portion. The inductance of the coil portions 525 and/or the impedance of the corresponding impedance unit 526 may differ in size between the respective receiving portions. This may cause receiving portions to have different resonance frequencies in relation to each other. A variable impedance of the impedance unit 526 may be individually controlled by a controller to change the resonance frequencies of the respective receiving portions. Each receiving portion may transfer different amounts of energy to the energy consuming device 528 depending on the resonance frequency of the receiving portion and the frequency of the transcutaneous transferred energy. By having different resonance frequencies of the receiving portions, a better energy transfer efficiency of the implantable medical device 565 may be obtained. Consecutive, sequential, or independent charging may be performed, where each receiving portion receives energy of different frequencies. Each receiving portion may have a resonance frequency adapted to different transcutaneously transferred energy frequencies, from one or more external units.

Advantages of having coil portions 525 include that it may reduce the required amount of coils and the amount of material needed. FIG. 65R illustrates sequential coil portions 525, where the coil portions 525 are adjacent but not overlapping. Coil portions 525 may overlap, be separate, or be partially overlapping segments of the receiving coil. This is illustrated in FIG. 65S.

FIG. 65S illustrates an implantable medical device 575. The implantable medical device 575 comprises an energy consuming part 528, a receiving circuit 523, and impedance units 526, similar to the energy consuming part 528, receiving circuit 523, and impedance units 526 of FIG. 65R, respectively. The implantable medical device of FIG. 65S may further comprise a measurement unit and/or a controller similar to the measurement unit 521 and the controller 520 of FIG. 65P, described above. Similar to the receiving unit 530 of FIG. 65P, the receiving unit 575 of FIG. 65S may be connected directly to the energy consuming part 528 or connected first to intermediate circuits, wherein the intermediate circuits are connected to the energy consuming part 528. Intermediate circuits may be the same as described for FIG. 65P above. The medical device 575 of FIG. 65S may be configured to receive energy from an external unit, such as the external unit 510 of FIG. 65P and/or the external unit 511 of FIG. 65Q.

The implantable medical device 575 further comprises coil portions 525, similar to the coil portions 525 of FIG. 65R, described above. FIG. 65S illustrates coil portions 525 that are overlapping with each other. Each coil portion 525 is connected to the respective impedance unit 526, as mentioned for the coil portions 525 and the impedance units 526 of FIG. 65R. An advantage of having overlapping coil portions 525 is that shorter or fewer coils may be used. Overlapping coil portions allow for larger inductances of the coil portions for a set receiver coil. Overlapping coil portions allows for a better energy transfer efficiency.

FIG. 65T illustrates an implantable medical device 585. The implantable medical device 585 comprises an energy consuming part 528, similar to the energy consuming part 528 of any of FIGS. 65P-65S. The implantable medical device 585 of FIG. 65T further comprise a first receiving unit 530, similar to the receiving unit 530 of FIGS. 65P and 65Q. The implantable medical device 585 of FIG. 65T further comprise a second receiving unit 535, similar to the receiving unit 535 of any of FIGS. 65R and 65S. The first receiving unit 530 and the second receiving unit 535 are electrically connected to the energy consuming part 528 by electrical connections 502. As mentioned for the receiving unit 530 of FIG. 65P, the receiving unit 585 of FIG. 65T may be connected directly to the energy consuming part 528 or connected first to intermediate circuits, wherein the intermediate circuits are connected to the energy consuming part 528. Intermediate circuits may be the same as described for FIG. 65P above.

FIG. 65T illustrates a implantable medical device 585 comprising one first receiving unit 530 and one second receiving unit 535. The implantable medical device 585 may comprise more than one first receiving unit 530. The implantable medical device 585 may comprise more than one second receiving unit 535. A mix of first receiving units 530 and second receiving units 535 may allow the implantable medical device 585 to be compact and customizable. The implantable medical device 585 may comprise more than one second receiving unit 535 and no first receiving units 530.

The implantable medical device 585 of FIG. 65T may further comprise a measurement unit and/or a controller similar to the measurement unit 521 and the controller 520 of FIG. 65P. The medical device 585 may be configured to receive energy from an external unit, such as the external unit 510 of FIG. 65P and/or the external unit 511 of FIG. 65Q.

In some examples, a coil comprised in the receiving unit 530 may comprise a plurality of windings. The plurality of windings may be connected to a respective variable impedance (as described above). An internal controller may control each of the variable impedances individually, thus providing for adjusting the resonant frequency of each of the windings separately. For examples, the secondary coil may comprise a first and a second winding, each connected to a respective variable impedance.

Large coil

A system for wirelessly charging an implantable medical implant, when implanted in a body of a patient is provided. The system comprises an internal energy receiver comprising a secondary coil, the internal energy receiver being connected to the implantable medical implant and an external energy transmitter comprising a primary coil for wirelessly transmitting energy to the internal energy receiver via the secondary coil. The diameter of the primary coil is larger than a diameter of the secondary coil.

According to embodiments described with reference to FIG. 65A-65C, the controller 300 of the implantable system 10 comprises a receiving unit 305 or internal energy receiver 305 comprising a secondary coil 192 (specifically shown in FIG. 65B′) configured for receiving transcutaneously transferred energy. The implantable system may receive the energy from an external device (also called an external energy transmitter), the external device being arranged outside of the body of the patient. The external device may comprise a primary coil for inducing a current in the coil 192 of the energy receiver 304 for wirelessly transfer energy to the receiving unit 305.

According to some embodiments, the primary coil is larger than the coil 192. By having the primary coil being larger than the secondary coil 192, the energy transmission may be improved. By having a diameter of the primary coil being larger than a diameter of the secondary coil, the wireless charging may be improved. For example, in previous wireless charging solution, there is a need for a great precision of arrangement of the secondary coil in relation to the primary coil. By having a larger diameter of the secondary coil, the need for precision may be reduced. Furthermore, having a larger primary coil wirelessly transmitting energy to a small secondary coil may provide for an improved energy transfer efficiency.

The implantable medical device may further comprise an internal controller connected to the internal energy receiver, for controlling the amount of energy received by the internal energy receiver. In some examples, the internal energy receiver further comprises a measurement unit for measuring a parameter related to the implantable medical implant or the body of the patient. The controller may be configured to measure the accumulated energy received by the internal energy receiver over a period of time and to measure a current change in energy received, and to control the energy received based on the accumulated energy and the current change. In some examples, the controlled comprises a Proportional—Integral—Derivative, PID, regulator for controlling the received energy.

The implantable medical device may comprise a variable impedance and/or a switch as described above.

With regards to the primary coil, the diameter of the primary coil may be more than 0.5 cm, more than 10 cm, more than 15 cm, more than 20 cm, more than 30 cm, or more than 50 cm. Alternatively, or in combination, the area of the primary coil is more than 0.5 cm2, more than 2 cm2, more than 10 cm2, more than 100 cm2, more than 300 cm2, more than 500 cm2, or more than 800 cm2.

Advantageously, any of the embodiments relating to wireless charging, for example, controlling energy transfer, PID regulation, variable impedance, large coil, and emergency backup function, among others, may be combined with any embodiment related to energy transfer described herein, for example Aspects 432, 433, 434, for an increased energy transfer safety mechanism.

### Emergency Backup Function

Another risk associated with an energized implantable medical device is that the implantable medical device's battery or energy storage is depleted and thus unable to energize the implantable medical device. Further, there is a risk that the internal energy receiver malfunctions, also resulting in a malfunction of the powering of the implantable medical device.

Thus, there is provided a safety mechanism that may be advantageously combined with any embodiment or aspect relating to an energized implantable medical implant described herein.

FIG. 65V shows a schematic illustration of an implantable medical device 100. The implantable medical device 100 may have an active portion 112 and an internal energy receiver 395. The active portion 112 may, for example, be configured to treat, monitor or perform a function of a body of a patient. The implantable medical implant 100 may comprise or be connected to a backup system 113, the backup system 113 being adapted to perform a backup function related to the active portion 112 of the implantable medical device 100.

In some examples, the backup function relates to switching a function of the active portion 112 off. The backup function may be any function relating to the function of the active portion, such as, but not limited to: opening an artificial sphincter, stopping a stretching a stomach portion, or stopping a stimulation of tissue. In some examples, the backup system is configured to reverse a function of the medical devices. For example, if the implanted medical device is used to constrict the urethra of a patient having urinary incontinence, the user must naturally be capable of opening said constriction, in order to perform urination, even if the implantable medical device 100 is malfunctioning.

The backup system 113 may, for example, comprise a backup energy receiver 114 to receive energy from an external device (such as any of the external devices or remote controls described herein), or to perform a function of the active portion. The backup energy receiver 114 may be adapted to receive wirelessly transferred energy from an external device (which may also be referred to as an external energy transmitter). To this end, the backup energy receiver may comprise a second secondary coil for receiving such energy. For example, in a case where the implantable medical device 100 malfunctions, an external device may wirelessly transfer energy to the backup energy receiver. The backup energy receiver 114 may receive the wirelessly transferred energy and the received energy may be used by the backup system 113 to perform the backup function.

In some examples, the function of the backup system 113 is to transfer the energy received via the backup energy receiver 114 for powering the medical device 100, or it may be used to charge a battery or accumulator of the medical device 100.

In some examples, the backup system 113 may use a battery or energy storage used by the active portion 112.

The backup function may be triggered by an external device 320″′. The external device may be any external device or remote control as described herein. The external device 320″′ may be adapted to wirelessly transfer energy to the backup system, and/or be configured to trigger the backup function of the backup system 113. The backup function may thus comprise an backup internal communications unit 115 for receiving a command from the external device, and be configured to execute the received command.

In some examples, the backup function may be triggered by an error detected by a measuring unit or a controller comprised in the medical implant. Such an error may, for example, be detected by a pressure being too high or too low, a temperature being too high or low, a battery charge status being too low, a measurement value deviating from a predetermined interval, or something else.

In other examples, a malfunction of the implantable medical device 100 may relate to the programming of the implantable medical device. In that case, the backup function of the backup system may be to re-program the malfunctioning program of the implantable medical device 100. The re-programming may be performed using any of the methods described herein.

In some examples, the backup energy receiver 114 comprises a passive or active RFID circuit adapted to be powered by the external device. In some examples, the backup energy receiver 114 comprises an NFMI energy receiver adapted to receive energy from the external device. The backup energy receiver 114 and the backup internal communication unit 115 may in some examples be comprised in the same unit, for example, in the cases where energy transmission and wireless communication may be performed using the same hardware.

### NFMI Communication and Wireless Energy Transfer

Any one of the medical devices described herein which utilize wireless communication in any way may be comprised in a system for communicating information from or to an implantable medical device, wherein the implantable medical device is implanted in a body of a patient. The system may comprise an internal communications unit comprised in or connected to the implantable medical device, and an external communications unit, wherein the internal communications unit and the external communications units are configured to send or receive data using near-field magnetic induction.

NFMI is a short-range wireless technology that communicates using a tightly coupled magnetic field. By the term NFMI it may be meant a short range wireless physical layer using low-power and non-propagating magnetic field. NFMI systems are designed to contain transmission energy within the localized magnetic field, and the magnetic field energy resonates around the communication system, but does not radiate into free space. The power density of near-field transmissions is restrictive and attenuates or rolls off at a rate proportional to the inverse of the range to the sixth power (1/r6) or −60 dB per decade. Thus, NFMI in the typical use only has a reach of around 1.5 to 2 meters.

NFMI signal can penetrate through human body tissue with low absorption rate. For example, the specific absorption rate (SAR) may be 100 times lower than Bluetooth. It has been realized that NFMI has a communication range through body tissue of for example 50 cm, which thus makes it advantageous to use for medical implants, as compared to RF communication which is disturbed by passing though body tissue. Thus, NFMI allows for communication with implants implanted also implanted deeper in the body.

Since NFMI has such a short rage, the possibility of an adversary to eavesdrop on communication with an implant, or to hack an implant form a distance is greatly reduced, as any adversary must be very close to the implant.

FIG. 65U shows an example of a system 600 using NFMI communication between an external communications unit 601 and an implantable medical device 603. The communications unit 602 is configured to communicate with the implantable medical device 603 through the skin 624 of the patient using NFMI communication. The implantable medical device comprises an internal communications unit 610 (which may alternatively be referred to as a receiving unit 305 in other embodiments) and an active portion 612. The active portion 612 may be configured to monitor, treat or perform a function of a body of a patient, and may be any medical device or medical implant described herein. The communications unit 601 may be comprised in any external device described herein.

The external communications unit 601 comprises an external coil 604 connected to an external NFMI transceiver 606. The external NFMI transceiver 606 which may comprise an NFMI transmitter chip. The external coil 604 and the external NFMI transceiver are configured to modulate a magnetic field for sending data and/or energy to the implantable medical device 603. The external NFMI may further comprise a capacitor for tuning.

In turn, the internal communications unit 610 may comprise an internal coil 614 and an internal NFMI transceiver 616. To receive data, the magnetic field modulated by the external coil 604 induces a voltage on the internal coil 614, which may be measured by the internal NFMI transceiver 616 and be decoded at the internal NFMI transceiver or at another part of the implantable medical device 603. The NFMI transceiver 616 may comprise an NFMI receiver chip. The NFMI receiver chip may comprise a tunable resistor and capacitor tank. Both of the tunable capacitance and resistance may vary within a certain range to automatically compensate the detuning of NFMI antennas.

It will be appreciated that a similar method may be used for sending data from the implantable medical device via the internal communications unit 616 to the communications unit 601 via the external communications unit 606. In that examples, the internal communications unit may comprise an NFMI transmitter chip similar to the NFMI transmitter chip comprised in the external device, and the external NFMI transceiver may comprise an NFMI receiver chip similar to the NFMI receiver chip comprised in the internal NFMI transceiver, connected to a respective coil for transmitting and/or receiving data.

Modulation schemes such as amplitude modulation, phase modulation and frequency modulation typically used in RF communications may be used in NFMI communication.

In some embodiments, the active portion is not a pacemaker, hearing aid or a neurostimulation implant.

The internal communications unit is adapted to be implanted at a tissue depth of at least 8 or 10 cm. For example, the internal communications unit may be adapted to be implanted in an abdomen of a patient.

Thus, any internal wireless communication unit comprised in an implant described herein may use NFMI to communicate with an external device. For example, for transmitting data, receiving data, receiving new programming or changes to the software of the implant and/or receiving control commands. The short rage of NFMI and the tissue depth at which NFMI may be used, makes it advantageous to use for any communication between an external device, such as a patient EID 320″, a patient remote device 320″′, a HCP EID, a HCP remote device, and an implantable medica device.

While the communications security between an implant and an external device is improved by the use of NFMI (as compared to RF communication), the information security may advantageously be combined with any encryption, data integrity checks or the like described herein.

For example, the internal communications unit may be configured to encrypt any data to be transmitted to the external communications unit, and the external communications unit may be configured to receive the data transmitted from the internal communications unit. In some examples, the external communications unit may be further configured to transmit that data to a server.

In a more specific example, NFMI may be used for wireless communication between an implant and a patient external interrogation device, patient EID, as described herein.

In some examples the external communications unit is configured to transmit a control command to the internal communications unit, and the internal communications unit is configured to transmit the control command to the implantable medical device. The control command may cause the implantable medical device to perform an action. The internal communications unit may, for example, be configured to transmit data, the data relating to a function of the implantable medical device or a measurement obtained by the implant.

The magnetic field may in addition to or as an alternative be used for charging or powering the medical implant. The use of NFMI for changing is an alternative or addition to any wireless charging of a medical implant described herein. In those cases, the internal communications unit is configured to store the received energy in a battery or similar, or to directly forward the received energy to the active portion 612 or another energy consuming part of the implantable medical device 603.

Using NFMI for charging a medical implant also has the advantage, compared to previous methods of charging an implant, that it is not heavily affected by passing through body tissue. For example, with the use of NFMI for charging, an implant at a tissue depth of 8 and up to 13 cm or more may be charged. This allows for practically charging an implant in almost any part of a body.

Advantageously, the NFMI communication system disclosed herein may be combined with any of aspects 250, 252, 255 and 284, and any of the embodiments described herein relating to wireless energy transfer using a coil.

According to one example, the system further comprises a second internal communications unit and a second external communications unit, wherein the second internal communications unit is adapted to receive and transmit data using a short range communications technology, and the second external communications unit is adapted to receive and transmit data using a short range communications technology, the short range communications technology having a shorter maximum range than NFMI. In one example, the short range communications technology is NFC, and the second internal communication unit comprises an NFC transceiver and the second external communication unit comprises an interrogation device for transmitting data to and from the RFID transceiver. By having these second internal and external communications unit, the implant may require a second authentication based on that the external communication unit is close to the implant, for example close enough to interrogate the NCF transceiver. Thus, it may be verified that the second external communication device is indeed close to the patient's body.

The implantable medical device 100 may be an active and/or operable implantable medical device 100 which may be an implantable medical device configured to exert a force on a body portion of the patient. The body portion of the patient may be a fluid carrying vessel, an organ, a joint, a membrane, a muscle, a bone or a nerve. The implantable medical device 100 may comprises an electrical motor and a controller for controlling the electrical motor and instructions transmitted to the implantable medical device 100 could be instructions pertaining to the control of the electrical motor. The controller may control, the speed, the acceleration or the torque of the motor.

A system for mitigating fibrin creation caused by the contact between the implantable medical device and the tissue or flowing blood of a patient, will now be described with reference to FIGS. 66a-72b. The system can be used as a coating for any implantable component of the medical devices disclosed herein, in particular enclosures, operable members and conduits and leads.

All foreign matter implanted into the human body inevitably causes an inflammatory response. In short, the process starts with the implanted medical device immediately and spontaneously acquiring a layer of host proteins. The blood protein-modified surface enables cells to attach to the surface enabling monocytes and macrophages to interact on the surface of the medical implant. The macrophages secrete proteins that modulate fibrosis and in turn developing the fibrosis capsule around the foreign body. In practice, a fibrosis capsule is a dense layer of excess fibrous connective tissue. On a medical device implanted in the abdomen, the fibrotic capsule typically grows to a thickness of about 0.5 mm-2 mm, and is substantially inelastic and dense.

The body tends to react to a medical implant, partly because the implant is a foreign object, and partly because the implant interacts mechanically with tissue of the body and/or blood flowing within the body. Implantation of medical devices and or biomaterial in the tissue of a patient may trigger the body's foreign body reaction (FBR). FBR leads to a formation of foreign body giant cells and the development of a fibrous capsule enveloping the implant. The formation of a dense fibrous capsule that isolates the implant from the host is the common underlying cause of implant failure. Implantation of medical devices and or biomaterial in a blood flow may also cause the formation of fibrous capsules due to the attraction of certain cells within the blood stream.

Implants may, due to the fibrin formation cause blood clotting leading to complications for the patient. Implants in contact with flowing blood and/or placed in the body may also lead to bacterial infection.

One common way of counteracting the creation of blood clots is by using blood thinners of different sorts. One commonly used blood thinner is called heparin. However, heparin have certain side-effects that are undesirable.

Fibrin is an insoluble protein that is partly produced in response to bleeding and is the major component of blood clots. Fibrin is formed by fibrinogen, a soluble protein that is produced by the liver and found in blood plasma. When tissue damage results in bleeding, fibrinogen is converted at the wound into fibrin by the action of thrombin, a clotting enzyme. The fibrin then forms, together with platelets, a hemostatic plug or clot over a wound site.

The process of forming fibrin from fibrinogen starts with the attraction of platelets.

Platelets have thrombin receptors on their surfaces that bind serum thrombin molecules. These molecules can in turn convert soluble fibrinogen into fibrin. The fibrin then forms long strands of tough and insoluble protein bound to the platelets. The strands of fibrin are then cross-linked so that it hardens and contracts, this is enabled by Factor XIII which is a zymogen found in the blood of humans.

FIGS. 66a-66c describes the reaction that takes place when a blood vessel is damaged. A blood vessel 700 is damaged and wound 710 appears. The blood contains many different cells and particles, for example red blood cells 720 and platelets 730. When the wound 710 appears red blood cells 720 and platelets 730 start to gather at the wound 710. Due to the thrombin receptors on the surface of the platelets 730 a fibrin sheath 740 starts to form which eventually creates a clot that stops the bleeding.

Fibrin may also be created due to the foreign body reaction. When a foreign body is detected in the body the immune system will become attracted to the foreign material and attempt to degrade it. If this degradation fails, an envelope of fibroblasts may be created to form a physical barrier to isolate the body from the foreign body. This may further evolve into a fibrin sheath, in case the foreign body is an implant this may hinder the function of the implant.

Implants can, when implanted in the body, be in contact with flowing blood. This may cause platelet adhesion on the surface of the implants. The platelets may then cause the fibrinogen in the blood to convert into fibrin creating a sheath on and or around the implant. This may prevent the implant from working properly and may also create blood clots that are perilous for the patient.

Implants not in contact with flowing blood can still malfunction due to fibrin creation. Here the foreign body reaction may be the underlying factor for the malfunction. Further, the implantation of a foreign body into the human body may cause an inflammatory response. The response generally persists until the foreign body has been encapsulated in a relatively dense layer of fibrotic connective tissue, which protects the human body from the foreign body. The process may start with the implant immediately and spontaneously acquiring a layer of host proteins. The blood protein-modified surface enables cells to attach to the surface, enabling monocytes and macrophages to interact on the surface of the implant. The macrophages secrete proteins that modulate fibrosis and in turn develop the fibrosis capsule around the foreign body, i.e., the implant. In practice, a fibrosis capsule may be formed of a dense layer of excess fibrous connective tissue. The inelastic properties of the fibrotic capsule may lead to hardening, tightness, deformity, and distortion of the implant, which in severe cases may result in revision surgery.

Any implant that is implanted into the body may trigger the formation of fibrin sheaths.

FIG. 67 shows a portion of implantable medical device 10 placed inside a vein 700 with a fibrin sheath 740 that has formed on and around part of the medical device 10. The sheath 740 may cause the implant 10 to malfunction and may further create a blood clot that may be harmful for the patient. The sheath 740 may cover the lumen 15 of the catheter 10 which inhibits the function of the implant 10. The formation of a blood clot may have several steps, as depicted in FIG. 68a-68d. FIG. 68a shows a fibrin sheath 740 created on a medical device 10 inside a vein 700. In FIG. 68b the sheath has further developed into an intraluminal clot 740. In FIG. 68c the clot has connected to one side of the vein creating a mural thrombosis 740. And in FIG. 68d the clot has reached a venous thrombosis 740.

A fibrin sheath 740 may be created on any implantable medical device 10 and may then cover certain necessary part of the device 10 inhibiting the function of the device 10.

Implants or biomaterials that are inserted to support or replace body parts may also cause infections of different sorts. Bacterial colonization that lead to implant-associated infections are a known issue for many types of implants. For example, the commensal skin bacteria, Staphylococci, and the Staphylococcus aureus tend to colonize foreign bodies such as implants and may cause infections. A problem with the Staphylococci is that it may also produce a biofilm around the implant encapsulating the bacterial niche from the outside environment. This makes it harder for the host defense systems to take care of the bacteria. There are other examples of bacteria and processes that creates bacteria causing infection due to implants.

FIG. 69 shows an implantable medical device or implant 10 comprising an implant surface 750 and a coating 760 arranged on the surface 750. The coating 760 may be configured to have antibacterial and/or antithrombotic characteristics. Depending on the use of the implantable medical device one or both of these effects may be advantageous. The coating 760 may be arranged on the surface 750 so that the coating shields the surface 750 from direct contact with the host body where the implantable medical device 10 is inserted.

The coating 760 may comprise at least one layer of a biomaterial. The coating 760 may comprise a material that is antithrombotic. The coating 760 may also comprise a material that is antibacterial. The coating 760 may be attached chemically to the surface 750.

FIG. 70 shows an exemplary implantable medical device or implant 10 comprising an at least partially hollow implant body 10. Blood may be arranged to flow within the implant body 10. The implant 10 may for example be a catheter. Since multiple surfaces of the implant 10 may be in contact with flowing blood it may comprise a first coating 760a and a second coating 760b. The coatings 760a and 760b may be similar or have different properties. Depending on how the implant 10 is placed the coatings 760a and 760b may come into contact with different parts or liquids within the body and may therefore comprise either similar materials or materials with different properties.

FIG. 71 shows an exemplary implantable medical device or implant 10 with a surface 750. The implantable medical device 10 comprises multiple coatings, 760a, 760b, 760c arranged on the surface. The implant 10 may comprise any number of coatings, the particular embodiment of FIG. 71 discloses three layers of coating 760a, 760b, 760c. The second coating 760b is arranged on the first coating 760a. The different coating 760a, 760b, 760c may comprise different materials with different features to prevent either fibrin sheath formation or bacteria gathering at the surface 750. As an example, the first coating 760a may comprise a layer of perfluorocarbon chemically attached to the surface. The second coating 760b may comprise a liquid perfluorocarbon layer arranged on the first coating 760a. Perfluorocarbon is used in medicine application in a variety of fields and may be advantageous for using as a coating layer.

The coatings may comprise any type of substance with antithrombotic, antiplatelet or antibacterial features. Such substances include sortase A, perfluorocarbon and more.

The coatings presented in relation to the figures may also be combined with an implantable medical device comprising certain materials that are antibacterial or antithrombotic. For example, some metals have shown to be antibacterial. In case the implant, or at least the surfaces of the implant, are made out of such a metal it may be advantageous in order to reduce bacterial infections. The medical implant or the surface of the implant may be made out of any other suitable metal or material.

The surface may for example comprise any of the following metals, or any combination of the following metals: titanium, cobalt, nickel, copper, zinc, zirconium, molybdenum, tin or lead.

An implantable medical device can also be coated with a slow releasing anti-fibrotic or antibacterial drug in order to prevent fibrin sheath creation and bacterial inflammation. The drug or medicament may be coated on the surface and be arranged to slowly be released from the implant in order to prevent the creation of fibrin or inflammation. The drug may also be covered in a porous or soluble material that slowly disintegrates in order to allow the drug to be administered into the body and prevent the creation of fibrin. The drug may be any conventional anti-fibrotic or antibacterial drug.

FIGS. 72a and 72b shows different micropatterns on the surface 750 of an implant. In order to improve blood compatibility, the implant materials physical structure may be altered or controlled. By creating a certain topography on the surface 750 of an implant fibrin creation and inflammatory reactions may be inhibited. FIG. 72a is an example of a micropattern that mimics the features of sharkskin. The micropattern may have many different shapes, many different depths into the surface 750 of the implant 10 and may be a complement to other coatings or be used individually. In FIG. 72b another example of a micropattern is disclosed.

The micropattern may for example be etched into the surface 750 of the implantable medical device 10 prior to insertion into the body. The surface of the implantable medical device 10 may for example comprise a metal. The surface may for example comprise any of the following metals, or any combination of the following metals: titanium, cobalt, nickel, copper, zinc, zirconium, molybdenum, tin or lead. This may be advantageous in that these metals have proven to be antibacterial which may ensure that the implant functions better when inserted into the host body.

The different aspects or any part of an aspect or different embodiments or any part of an embodiment may all be combined in any possible way. Any method or any step of method may be seen also as an apparatus description, as well as, any apparatus embodiment, aspect or part of aspect or part of embodiment may be seen as a method description and all may be combined in any possible way down to the smallest detail. Any detailed description should be interpreted in its broadest outline as a general summary description, and please note that any embodiment or part of embodiment as well as any method or part of method could be combined in any way. All examples herein should be seen as part of the general description and therefore possible to combine in any way in general terms.

In the following, numbered aspect groups 343-351, 368 and 369 of the present invention are provided. The different aspects are numbered individually within the groups and the references to other aspects relate to aspects within the same group. The scope of protection is however defined by the appended claims.

### Aspect 343—Obesity_Stretch_Version2_Adaptable

1. A medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of: adapting the medical device to the curvature of the stomach of the patient, and facilitating insertion of the medical device into the body of the patient.
2. The medical device according to aspect 1, wherein the operation device is further configured for operating the second member to displace the second member in a third direction relative to the first member, for stretching the stomach wall between the first and second portion.
3. The medical device according to any one of the preceding aspects, further comprising a third member configured to be fixated to a third portion of the stomach wall.
4. The medical device according to aspect 3, wherein the operation device is further configured for operating the third member to displace the third member relative to at least one of the first and second member, for stretching the stomach wall between the third portion and at least one of the first and second portions.
5. The medical device according to any one of aspects 3-4, further comprising a fourth member configured to be fixated to a fourth portion of the stomach wall.
6. The medical device according to aspect 5, wherein the operation device is further configured for operating the fourth member to displace the fourth member relative to at least one of the first, second and third member, for stretching the stomach wall between the fourth portion and at least one of the first, second and third portions.
7. The medical device according to any one of the preceding aspects, wherein at least one of the first, second, third and fourth member is inoperably fixated to a main portion.
8. The medical device according to aspect 7, wherein at least two of the first, second, third and fourth members are inoperably fixated the main portion.
9. The medical device according to any one of aspects 1-6, wherein at least one of the first, second, third and fourth member is operably fixated to a main portion.
10. The medical device according to aspect 9, wherein at least two of the first, second, third and fourth members are operably fixated the main portion.
11. The medical device according to any one of aspects 7-10, wherein the main portion comprises at least part of the operation device.
12. The medical device according to any one of the preceding aspects, wherein at least one of the first, second, third and fourth member comprises at least part of the operation device.
13. The medical device according to aspect 7, wherein the member being inoperably fixated to the main portion comprises at least part of the operation device.
14. The medical device according to aspect 8, wherein the two members being inoperably fixated to the main portion each comprises at least part of the operation device.
15. The medical device according to aspect 8, wherein the first member is inoperably fixated to the main portion and comprises a first portion of the operation device, and the second member is inoperably fixated to the main portion and comprises a second portion of the operation device, and wherein the first portion of the operation device is configured for operating the third member and the second portion of the operation device is configured for operating the fourth member.
16. The medical device according to aspect 7, wherein the first member is inoperably fixated to the main portion and comprises a first portion of the operation device, and a second portion of the operation device, wherein the first portion of the operation device is configured for operating the third member and the second portion of the operation device is configured for operating the fourth member.
17. The medical device according to any one of the preceding aspects, wherein at least one of the first, second, third and fourth member is elongated.
18. The medical device according to any one of the preceding aspects, wherein at least one of the first, second, third and fourth member is hinged for enabling pivotal movement.
19. The medical device according to any one of the preceding aspects, wherein at least one of the first, second, third and fourth member is configured to be at least partially invaginated by the tissue of the stomach wall.
20. The medical device according to any one of the preceding aspects, wherein at least one of the first, second, third and fourth member comprises at least one fixation portion configured for fixation of the member to tissue of the stomach wall using at least one of sutures, staplers or tissue growth promoting structure.
21. The medical device according to aspect 20, wherein the at least one fixation portion comprises at least one of a through-hole and a recess for receiving the at least one of sutures or staplers.
22. The medical device according to any one of the preceding aspects, wherein the operation device is a mechanical operation device.
23. The medical device according to any one of the preceding aspects, wherein the operation device is a hydraulic operation device.
24. The medical device according to any one of the preceding aspects, wherein the operation device comprises at least one electrical motor.
25. The medical device system according to aspect 24, wherein the operation device further comprises a gear system configured to reduce the speed and increase the force of the movement generated by the electrical motor.
26. The medical device according to any one of the preceding aspects, wherein the operation device is configured to be remotely powered.
27. The medical device according to aspect 26, wherein the operation device is configured to be remotely powered with mechanical force.
28. The medical device according to aspect 27, further comprising: a receiving portion configured to receive mechanical force, and a transmission for transforming the received mechanical force into a force for displacing at least one of the members for stretching the stomach wall.
29. The medical device according to aspect 28, wherein the receiving portion is configured to receive a rotating mechanical force, and wherein the transmission is configured to transform the received rotating mechanical force into a liner mechanical force.
30. The medical device according to aspect 28, wherein the receiving portion is configured to receive a linear mechanical force.
31. The medical device according to aspect 26, wherein the operation device is configured to be remotely powered with hydraulic force.
32. The medical device according to aspect 31, further comprising: a receiving portion configured to receive hydraulic force, and a transmission for transforming the received hydraulic force into a force for displacing at least one of the members for stretching the stomach wall.
33. The medical device according to aspect 32, wherein the transmission comprises at least one hydraulic cylinder.
34. The medical device according to any one of the preceding aspects, further comprising an enclosure configured to enclose at least a portion of the operation device, and wherein the enclosure is flexible to enable the displacement of at least one member.
35. The medical device according to aspect 34, wherein the enclosure comprises the main portion.
36. The medical device according to any one of the preceding aspects, further comprising an energy storage unit for directly or indirectly energizing the medical device.
37. The medical device according to any one of the preceding aspects, further comprising a controller for controlling the operation device.
38. The medical device according to aspect 37, wherein the controller comprises a wireless transceiver for communicating wirelessly with a source external to the body of the patient.
39. The medical device according to aspect 38, further comprising at least one sensor, and wherein the controller is configured for receiving sensor input from the at least one sensor.
40. The medical device according to aspect 39, wherein the sensor is a sensor configured to sense a physical parameter of the medical device system.
41. The medical device according to aspect 40, wherein the sensor is a sensor configured to sense at least one of:
   a temperature of the medical device system,
   a parameter related to the power consumption of the medical device system,
   a parameter related to strain in the medical device system,
   a parameter related to a status of the energy storage unit,
   a parameter related to the wireless transfer of energy from a source external to the body of the patient,
   a hydraulic pressure.
42. The medical device according to aspect 39, wherein the sensor is a sensor configured to sense a physiological parameter of the patient.
43. The medical device according to aspect 42, wherein the sensor is a sensor configured to sense at least one of:
a parameter related to the patient swallowing,
a local temperature,
a systemic temperature,
blood saturation,
blood oxygenation,
blood pressure,
a parameter related to an ischemia marker, or pH.
44. The medical device according to aspect 43, wherein the sensor configured to sense a parameter related to the patient swallowing comprises at least one of:
   a motility sensor,
   a acoustic sensor,
an optical sensor, or
a strain sensor.
45. The medical device according to aspect 43, wherein the sensor configured to sense pH is configured to sense the acidity in the stomach.
46. The medical device according to any one of aspects 39-45, wherein the controller is configured to transmit information based on sensor input to a source external to the body of the patient.
47. The medical device according to aspect 46, wherein the controller is configured to transmit the information based on sensor input to a source external to the body of the patient, wirelessly.
48. The medical device according to any one of the preceding aspects, further comprising a capacitor connected to the implantable energy storage unit and connected to the operation device, wherein the capacitor is configured to be charged by the implantable energy storage unit and to provide the operation device with electrical power.
49. The medical device system according to any one of aspects 36-48, wherein the implantable energy storage unit is a solid-state battery.
50. The medical device system according to aspect 49, wherein the battery is a tionyl-chlorid battery.
51. The medical device according to any one of the preceding aspects, further comprising a sensation generator adapted to generate a sensation detectable by a sense of the patient.
52. The medical device according to any one of aspects 37-51, wherein the controller is configured to receive a patient generated control signal.
53. The medical device according to aspect 52, wherein the controller is configured to control the operation device on the basis of the received patient generated control signal.
54. The medical device according to any one of aspects 37-53, wherein the controller is configured to control the operation device on the basis of a signal related to a lapsed time or a time of day.
55. The medical device according to any one of aspects 37-54, wherein the controller is configured to receive a signal from a sensor external to the body of the patient.
56. The medical device according to any one of the preceding aspects, further comprising an electrode arrangement configured to be arranged to engage and electrically stimulate muscle tissue of the stomach to exercise the muscle tissue to improve the conditions for long term implantation of the medical device.
57. A medical device system comprising: the medical device according to any one of aspects 1-56, and an implantable remote unit connected to the medical device.
58. The medical device system according to aspect 57, wherein the implantable remote unit comprises an energy storage unit for directly or indirectly energizing the medical device.
59. The medical device system according to any one of aspects 57 and 58, wherein the implantable remote unit comprises an energy receiver for wirelessly receiving energy from a source external to the body of the patient.
60. The medical device system according to any one of aspects 57-59, wherein the implantable remote unit comprises a controller for controlling the medical device system.
61. The medical device system according to aspect 60, wherein the controller comprises a wireless transceiver for communicating wirelessly with a source external to the body of the patient.
62. The medical device system according to any one of aspects 60 and 61, wherein the implantable remote unit further comprises at least one sensor, and wherein the controller is configured for receiving sensor input from the at least one sensor.
63. The medical device system according to aspect 62, wherein the sensor is a sensor configured to sense a physical parameter of the medical device system.
64. The medical device system according to aspect 63, wherein the sensor is a sensor configured to sense
at least one of:
a temperature of the medical device system,
a parameter related to the power consumption of the medical device system,
a parameter related to strain in the medical device system,
a parameter related to a status of the energy storage unit,
a parameter related to the wireless transfer of energy from a source external to the body of the patient,
a hydraulic pressure.
65. The medical device system according to aspect 62, wherein the sensor is a sensor configured to sense a physiological parameter of the patient.
66. The medical device system according to aspect 65, wherein the sensor is a sensor configured to sense
at least one of:
a parameter related to the patient swallowing,
a local temperature,
a systemic temperature,
blood saturation,
blood oxygenation,
blood pressure,
a parameter related to an ischemia marker, or
pH.
67. The medical device system according to aspect 66, wherein the sensor configured to sense a parameter related to the patient swallowing comprises at least one of:
   a motility sensor,
   a acoustic sensor,
an optical sensor, or
a strain sensor.
68. The medical device system according to aspect 66, wherein the sensor configured to sense pH is configured to sense the acidity in the stomach.
69. The medical device system according to any one of aspects 62-68, wherein the controller is configured to transmit information based on sensor input to a source external to the body of the patient.
70. The medical device system according to aspect 69, wherein the controller is configured to transmit the information based on sensor input to a source external to the body of the patient, wirelessly.
71. The medical device system according to any one of aspects 57-70, wherein the implantable remote unit comprises at least a portion of the operation device.
72. The medical device system according to aspect 71, wherein the portion of the operation device comprises at least one electrical motor.
73. The medical device system according to aspect 72, wherein the portion of the operation device further comprises a gear system configured to reduce the speed and increase the force of the movement generated by the electrical motor.
74. The medical device system according to any one of aspects 71-73, wherein the portion of the operation device comprises at least one hydraulic pump.
75. The medical device system according to any one of aspects 58-74, further comprising a capacitor connected to the implantable energy storage unit and connected to the operation device, wherein the capacitor is configured to be charged by the implantable energy storage unit and to provide the operation device with electrical power.
76. The medical device system according to any one of aspects 58-75, wherein the implantable energy storage unit is a solid-state battery.
77. The medical device system according to aspect 76, wherein the battery is a tionyl-chlorid battery.
78. The medical device system according to any one of aspects 57-77, further comprising a sensation generator adapted to generate a sensation detectable by a sense of the patient.
79. The medical device system according to any one of aspects 60-78, wherein the controller is configured to receive a patient generated control signal.
80. The medical device system according to aspect 79, wherein the controller is configured to control the medical device system on the basis of the received patient generated control signal.
81. The medical device system according to any one of aspects 60-80, wherein the controller is configured to control the medical device system on the basis of a signal related to a lapsed time or a time of day.
82. The medical device system according to any one of aspects 60-81, wherein the controller is configured to receive a signal from a sensor external to the body of the patient.
83. The medical device system according to any one of aspects 57-82, further comprising a force transferring element configured to mechanically transfer force from the implantable remote unit to the medical device.
84. The medical device system according to aspect 83, wherein the force transferring element is configured to transfer a rotating mechanical force from the implantable remote unit to the medical device.
85. The medical device system according to aspect 83, wherein the force transferring element is configured to transfer a linear mechanical force from the implantable remote unit to the medical device.
86. The medical device system according to any one of aspects 57-82, further comprising a force transferring element configured to hydraulically transfer force from the implantable remote unit to the medical device.
87. The medical device system according to any one of aspects 57-86, further comprising at least one lead for transferring electrical energy and/or information from the implantable remote unit to the medical device.
88. The medical device system according to any one of aspects 57-87, further comprising at least one injection port for injecting fluid into the medical device system.
89. A method of implanting a medical device for actively stretching a stomach wall of a patient for creating a sensation of satiety, the medical device comprising a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions, wherein at least one of the first and second member is flexible in a second direction substantially perpendicular to the first direction, the method comprising:
making an incision in the abdomen of the patient, for accessing the stomach, dissecting a portion of the stomach,
inserting the medical device into the abdomen of the patient, using the flexibility of at least one of the first and second member for adapting the medical device to the curvature of the stomach of the patient, and
fixating the first member to a first portion of the stomach wall, and fixating the second member to a second portion of the stomach wall.
90. The method according to aspect 89, wherein the medical device further comprises a third member configured to be fixated to a third portion of the stomach wall, and wherein the method comprises the step of fixating the third member to a third portion of the stomach wall.
91. The method according to aspect 90, wherein the medical device further comprises a fourth member configured to be fixated to a fourth portion of the stomach wall, and wherein the method comprises the step of fixating the fourth member to a fourth portion of the stomach wall.
92. The method according to any one of aspects 89-91, wherein at least one of the first, second, third and fourth members are connected to a main portion, and wherein the method comprises using flexibility of at least one of the first, second, third and fourth members for adapting the medical device to the curvature of the stomach of the patient by pivoting at least one of the first, second, third and fourth members relative to the main portion.
93. The method according to any one of aspect 89-92, wherein at least one of the steps of fixating the first member to a first portion of the stomach wall, and fixating the second member to a second portion of the stomach wall comprises at least partially invaginated at least one of the first and second member by the tissue of the stomach wall.
94. The method according to any one of aspect 89-93, wherein at least one of the steps of fixating the first member to a first portion of the stomach wall, and fixating the second member to a second portion of the stomach wall comprises fixating at least one of the first and second member to tissue of the stomach wall using at least one of sutures, staplers or tissue growth promoting structure.
95. The method according to any one of aspect 89-94, further comprising placing at least one sensor in the area of the esophagus of the patient for sensing a parameter related to the patient swallowing.
96. The method according to aspect 95, wherein the step of placing at least one sensor in the area of the esophagus of the patient for sensing a parameter related to the patient swallowing comprises placing at least one of: a motility sensor, a acoustic sensor, an optical sensor or a strain sensor.
97. The method according to any one of aspect 89-96, further comprising placing at least one sensor in the area of the stomach of the patient for sensing a parameter related to the patient eating.
98. The method according to aspect 97, wherein the step of placing at least one sensor in the area of the stomach of the patient for sensing a parameter related to the patient eating comprises placing at least one of temperature sensor, blood saturation or oxygenation sensor, a blood pressure sensor, a sensor configured to sense a parameter related to an ischemia marker, or a pH sensor.
99. The method according to aspect 98, wherein the step of sensing pH comprises sensing the acidity in the stomach.
100. The method according to any one of aspect 89-99, further comprising placing an electrode arrangement configured to be arranged between the medical device and the stomach to engage and electrically stimulate muscle tissue of the stomach to exercise the muscle tissue to improve the conditions for long term implantation of the medical device.
100. The method according to any one of aspect 89-100, further comprising placing an implantable remote unit connected to the medical device in the body of the patient.
101. The method according to aspect 100, wherein the step of placing an implantable remote unit connected to the medical device in the body of the patient comprises placing an implantable remote unit comprising at least a portion of the operation device.
102. The method according to any one of aspects 100 and 101, wherein the step of placing an implantable remote unit connected to the medical device in the body of the patient comprises placing an implantable remote unit comprising an energy storage unit.
103. The method according to any one of aspects 100-102, wherein the step of placing an implantable remote unit connected to the medical device in the body of the patient comprises placing an implantable remote unit comprising a wireless energy receiver.
104. The method according to any one of aspects 100-103, wherein the step of placing an implantable remote unit connected to the medical device in the body of the patient comprises placing an implantable remote unit comprising a wireless transceiver for wireless communication.
105. A method of implanting a medical device for actively stretching a stomach wall of a patient for creating a sensation of satiety, the medical device comprising a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions, wherein at least one of the first and second member is flexible in a second direction substantially perpendicular to the first direction, the method comprising:
making an incision in the abdomen of the patient, for accessing the stomach, dissecting a portion of the stomach,
using the flexibility of at least one of the first and second member for facilitating insertion of the medical implant into the body of the patient,
inserting the medical device into the abdomen of the patient.
106. The method according to aspect 105, wherein the step of using the flexibility of at least one of the first and second member for facilitating insertion of the medical device into the body of the patient comprises using the flexibility of at least one of the first and second members for compressing the medical device such that it can be inserted through an incision in the skin of the patient.
107. The method according to aspect 105, wherein the step of using the flexibility of at least one of the first and second member for facilitating insertion of the medical device into the body of the patient comprises using the flexibility of at least one of the first and second members for compressing the medical device such that it can be inserted through a cannula of a trocar and into the body of the patient.
108. The method according to any one of aspects 105-107, further comprising the step of using the flexibility of at least one of the first and second member for expanding the medical device after the medical device has been inserted into the body of the patient.
109. The method according to any one of aspects 105-108, further comprising the steps of fixating the first member to a first portion of the stomach wall, and fixating the second member to a second portion of the stomach wall.
110. The method according to any one of aspects 105-109, wherein the medical device further comprises a third member configured to be fixated to a third portion of the stomach wall, and wherein the method comprises the step of fixating the third member to a third portion of the stomach wall.
111. The method according to aspect 110, wherein the medical device further comprises a fourth member configured to be fixated to a fourth portion of the stomach wall, and wherein the method comprises the step of fixating the fourth member to a fourth portion of the stomach wall.
112. The method according to any one of aspects 105-111, wherein at least one of the first, second, third and fourth members are connected to a main portion, and wherein the method comprises using flexibility of at least one of the first, second, third and fourth members by pivoting at least one of the first, second, third and fourth members relative to the main portion.
113. The method according to any one of aspect 105-111 wherein at least one of the steps of fixating the first member to a first portion of the stomach wall, and fixating the second member to a second portion of the stomach wall comprises at least partially invaginating at least one of the first and second member by the tissue of the stomach wall.
114. The method according to any one of aspect 105-113, wherein at least one of the steps of fixating the first member to a first portion of the stomach wall, and fixating the second member to a second portion of the stomach wall comprises fixating at least one of the first and second member to tissue of the stomach wall using at least one of sutures, staplers or tissue growth promoting structure.
115. The method according to any one of aspect 105-114, further comprising placing at least one sensor in the area of the esophagus of the patient for sensing a parameter related to the patient swallowing.
116. The method according to aspect 115, wherein the step of placing at least one sensor in the area of the esophagus of the patient for sensing a parameter related to the patient swallowing comprises placing at least one of: a motility sensor, a acoustic sensor, an optical sensor or a strain sensor.
117. The method according to any one of aspect 105-116, further comprising placing at least one sensor in the area of the stomach of the patient for sensing a parameter related to the patient eating.
118. The method according to aspect 117, wherein the step of placing at least one sensor in the area of the stomach of the patient for sensing a parameter related to the patient eating comprises placing at least one of temperature sensor, blood saturation or oxygenation sensor, a blood pressure sensor, a sensor configured to sense a parameter related to an ischemia marker, or a pH sensor.
119. The method according to aspect 118, wherein the step of sensing pH comprises sensing the acidity in the stomach.
120. The method according to any one of aspect 105-119, further comprising placing an electrode arrangement configured to be arranged between the medical device and the stomach to engage and electrically stimulate muscle tissue of the stomach to exercise the muscle tissue to improve the conditions for long term implantation of the medical device.
121. The method according to any one of aspect 105-120, further comprising placing an implantable remote unit connected to the medical device in the body of the patient.
122. The method according to aspect 121, wherein the step of placing an implantable remote unit connected to the medical device in the body of the patient comprises placing an implantable remote unit comprising at least a portion of the operation device.
123. The method according to any one of aspects 121 and 122, wherein the step of placing an implantable remote unit connected to the medical device in the body of the patient comprises placing an implantable remote unit comprising an energy storage unit.
124. The method according to any one of aspects 121-123, wherein the step of placing an implantable remote unit connected to the medical device in the body of the patient comprises placing an implantable remote unit comprising a wireless energy receiver.
125. The method according to any one of aspects 121-124, wherein the step of placing an implantable remote unit connected to the medical device in the body of the patient comprises placing an implantable remote unit comprising a wireless communication unit.
126. A method of calibrating a medical device for actively stretching a stomach wall of a patient for creating a sensation of satiety, the medical device comprising a first member configured to be fixated to a first portion of the stomach wall, a second member configured to be fixated to a second portion of the stomach wall and an operation device for operating the second member to displace the second member in a first direction relative to the first member, for stretching the stomach wall between the first and second portions, and a controller comprising a transceiver for wireless communication, the method comprising:
   receiving, at the controller, a first input signal comprising at least one of: a sensor input signal related to a physical parameter of the medical device, and
an input signal from a source external to the body of the patient,
   controlling, by the controller, the operation device to adjust the stretching of the stomach wall, in response to the first input signal, and
   receiving, at the transceiver, a second input signal from the source external to the patient, and
   controlling, by the controller, the operation device to further adjust the stretching of the stomach wall, in response to the second input signal.
127. The method according to aspect 126, wherein the sensor input signal related to a physical parameter of the medical device is a sensor input related to at least one of:
   energy consumption,
position of at least one of the first member, the second member and the operation device, strain on at least one of the first member, the second member and the operation device, and speed of the operation device.
128. The method according to aspect 126, wherein the input signal from a source external to the body of the patient is an input signal generated by the patient.
129. The method according to aspect 128, wherein the input signal generated by the patient is an input signal related to at least one of:
a feeling of satiety experienced by the patient,
a feeling of pain experienced by the patient, and
the patient ingesting something.
130. The method according to any one of aspects 126-129, wherein the step of adjusting the stretching of the stomach wall comprises adjusting one of:
the force with which the stomach wall is stretched,
the speed with which the stomach wall is stretched,
the duration of the stretching of the stomach wall.
131. The medical device according to any one if aspects 1-130, wherein the operation device comprises a memory metal or an electroactive polymer.

### Aspect 344—Obesity_Stretch_Version2_Eccentric

1. A medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
2. The medical device according to aspect 1, wherein the at least one eccentric rotatable engagement member is configured to engage the second member at a distance from the point of pivot of the second member.
3. The medical device according to any one of aspects 1 and 2, wherein
   the eccentric rotatable engagement member comprises at least one of a recess or protrusion, and
   the second member comprises at least one of a recess or protrusion, and wherein the engagement between the recess or protrusion of the engagement member and the recess or protrusion of the second member causes the second member to pivot in relation to the main portion for displacing the second member in the first direction relative to the first member.
4. The medical device according to any one of the preceding aspects, wherein the first member is pivotally connected to the main portion, such that the first member can be displaced relative to at least one of the second member and a third member, by the first member pivoting in relation to the main portion.
5. The medical device according to any one of the preceding aspects, wherein the operation device is configured to simultaneously displace the second member in the first direction and the first member in a second direction to thereby increase the distance between the first stomach engager portion and the second stomach engager portion to stretch the stomach wall between the first portion of the stomach wall and the second portion of the stomach wall.
6. The medical device according to any one of the preceding aspects, wherein the operation device comprises a radial engagement surface configured to radially engage at least the second member such that the eccentric rotation of the engagement member causes the second member to pivot in relation to the main portion.
7. The medical device according to any one of the preceding aspects, wherein at least the second member is configured to be operated in the first direction by the eccentric rotation of the engagement member and in an opposite direction by an elastic element.
8. The medical device according to any one of the preceding aspects, wherein the eccentric rotatable engagement member comprises a groove encircling the center of rotation of the eccentric rotatable engagement member.
9. The medical device according to aspect 8, wherein the groove is configured to control the movement of at least the second member in the first direction and in the opposite direction.
10. The medical device according to aspect 8, wherein the groove is configured to control the movement of at least the second member at all times.
11. The medical device according to any one of aspects 8-10, wherein the medical device comprises the first member, the second member, the third member and a fourth member, and wherein:
   the first, second, third and fourth members are all pivotally connected to the main portion,
   the groove is configured to control the movement of the first, second, third and fourth members in a first direction and in an opposite direction, and
   as the eccentric rotatable engagement member comprising the groove rotates, the first, second, third and fourth members are sequentially displaced in relation to the main portion in the first direction and in the opposite direction for sequentially stretching the stomach wall portions between the members.
12. The medical device according to aspect 11, as the eccentric rotatable engagement member comprising the groove rotates:
   the first and second members are simultaneously displaced, or
   the second and third members are simultaneously displaced, or
   the third and fourth members are simultaneously displaced.
13. The medical device according to any one of the preceding aspects, wherein at least one of the first, second, third and fourth member is configured to be at least partially invaginated by the tissue of the stomach wall.
14. The medical device according to any one of the preceding aspects, wherein at least one of the first, second, third and fourth member comprises at least one fixation portion configured for fixation of the member to tissue of the stomach wall using at least one of sutures, staplers or tissue growth promoting structure.
15. The medical device according to aspect 14, wherein the at least one fixation portion comprises at least one of a through-hole and a recess for receiving the at least one of sutures or staplers.
16. The medical device according to any one of the preceding aspects, wherein the operation device comprises at least one electrical motor.
17. The medical device system according to aspect 16, wherein the operation device further comprises a gear system configured to reduce the speed and increase the force of the movement generated by the electrical motor.
18. The medical device according to any one of the preceding aspects, wherein the operation device is configured to be remotely powered.
19. The medical device according to aspect 18, wherein the operation device is configured to be remotely powered with mechanical force.
20. The medical device according to any one of the preceding aspects, further comprising an enclosure configured to enclose at least a portion of the operation device, and wherein the enclosure is flexible to enable the displacement of at least one member.
21. The medical device according to any one of the preceding aspects, further comprising an energy storage unit for directly or indirectly energizing the medical device.
22. The medical device according to any one of the preceding aspects, further comprising a controller for controlling the operation device.
23. The medical device according to aspect 22, wherein the controller comprises a wireless transceiver for communicating wirelessly with a source external to the body of the patient.
24. The medical device according to aspect 23, further comprising at least one sensor, and wherein the controller is configured for receiving sensor input from the at least one sensor.
25. The medical device according to aspect 24, wherein the sensor is a sensor configured to sense a physical parameter of the medical device system.
26. The medical device according to aspect 25, wherein the sensor is a sensor configured to sense at least one of:
a temperature of the medical device system,
a parameter related to the power consumption of the medical device system,
a parameter related to strain in the medical device system,
a parameter related to a status of the energy storage unit, or
a parameter related to the wireless transfer of energy from a source external to the body of the patient.
27. The medical device according to aspect 26, wherein the sensor is a sensor configured to sense a physiological parameter of the patient.
28. The medical device according to aspect 27, wherein the sensor is a sensor configured to sense at least one of:
a parameter related to the patient swallowing,
a local temperature,
a systemic temperature,
blood saturation,
blood oxygenation,
blood pressure,
a parameter related to an ischemia marker, or
pH.
29. The medical device according to aspect 28, wherein the sensor configured to sense a parameter related to the patient swallowing comprises at least one of:
   a motility sensor,
   a acoustic sensor,
   an optical sensor, or
   a strain sensor.
30. The medical device according to aspect 28, wherein the sensor configured to sense pH is configured to sense the acidity in the stomach.
31. The medical device according to any one of aspects 22-30, wherein the controller is configured to transmit information based on sensor input to a source external to the body of the patient.
32. The medical device according to any one of the preceding aspects, further comprising a capacitor connected to the implantable energy storage unit and connected to the operation device, wherein the capacitor is configured to be charged by the implantable energy storage unit and to provide the operation device with electrical power.
33. The medical device system according to any one of aspects 21-32, wherein the implantable energy storage unit is a solid-state battery.
34. The medical device system according to aspect 33, wherein the battery is a tionyl-chlorid battery.
35. The medical device according to any one of the preceding aspects, further comprising a sensation generator adapted to generate a sensation detectable by a sense of the patient.
36. The medical device according to any one of aspects 22-35, wherein the controller is configured to receive a patient generated control signal.
37. The medical device according to aspect 36, wherein the controller is configured to control the operation device on the basis of the received patient generated control signal.
38. The medical device according to any one of aspects 22-37, wherein the controller is configured to control the operation device on the basis of a signal related to a lapsed time or a time of day.
39. The medical device according to any one of aspects 22-38, wherein the controller is configured to receive a signal from a sensor external to the body of the patient.
40. The medical device according to any one of the preceding aspects, further comprising an electrode arrangement configured to be arranged to engage and electrically stimulate muscle tissue of the stomach to exercise the muscle tissue to improve the conditions for long term implantation of the medical device.
41. The medical device according to any one if aspects 1-40, wherein the operation device comprises a memory metal or an electroactive polymer.

### Aspect 345—Obesity_Stretch_Version2_Hydraulic-Arms

1. A medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
2. The medical device according to aspect 1, wherein the flexible wall portion comprises elevated and lowered portions.
3. The medical device according to aspect 2, wherein the elevated and lowered portions are configured to enable the second member to bend or pivot when fibrotic tissue is covering the elevated and lowered portions.
4. The medical device according to any one of aspects 1-3, wherein the flexible wall portion comprises at least one pleated portion.
5. The medical device according to any one of aspects 1-3, wherein the flexible wall portion comprises a bellows.
6. The medical device according to any one of the preceding aspects, wherein the second member comprises a pivoting element pivotally fixated to the main portion, and wherein the flexible wall portion causes the pivoting element to pivot when hydraulic fluid is moved to or from the fluid chamber for stretching the stomach wall between the first and second portions.
7. The medical device according to any one of the preceding aspects, wherein the second member comprises a second fluid chamber partially enclosed by a flexible wall portion, and wherein the bending or pivoting of the second member is caused by the altering the relationship between the fluid levels in the first and second fluid chamber.
8. The medical device according to aspect 7, wherein the bending or pivoting of the second member is caused by at least one of:
   movement of fluid from the first fluid chamber, and
   movement of fluid to the second fluid chamber.
9. The medical device according to any one of aspects 7 and 8, wherein the second member is configured to be displaced:
   in the first direction relative to the first member by the movement of fluid from the first fluid chamber, and
   in a second direction relative to the first member by the movement of fluid from the second fluid chamber.
10. The medical device according to any one of aspects 7 and 8, wherein the second member is configured to be displaced:
   in the first direction relative to the first member by the movement of fluid to the first fluid chamber, and
   in a second direction relative to the first member by the movement of fluid to the second fluid chamber.
11. The medical device according to any one of the preceding aspects, wherein the first member comprises a fluid chamber at least partially enclosed by a flexible wall portion, and wherein the flexible wall portion causes the first member to bend or pivot when hydraulic fluid is moved to or from the fluid chamber.
12. The medical device according to any one of the preceding aspects, wherein the medical device comprises at least one of:
   a third member comprising a fluid chamber at least partially enclosed by a flexible wall portion, and wherein the flexible wall portion causes the third member to bend or pivot when hydraulic fluid is moved to or from the fluid chamber,
   a fourth member comprising a fluid chamber at least partially enclosed by a flexible wall portion, and wherein the flexible wall portion causes the fourth member to bend or pivot when hydraulic fluid is moved to or from the fluid chamber, and
   a fifth member comprising a fluid chamber at least partially enclosed by a flexible wall portion, and wherein the flexible wall portion causes the fifth member to bend or pivot when hydraulic fluid is moved to or from the fluid chamber.
13. The medical device according to any one of the preceding aspects, further comprising at least one operable valve configured to operate to at least one of:
   switch between distributing hydraulic fluid to or from the fluid chamber of the first member and to or from the fluid chamber of the second member,
   switch between distributing hydraulic fluid to or from the fluid chamber of the first member and to or from the fluid chamber of the second member and to or from the fluid chamber of the third member,
   switch between distributing hydraulic fluid to or from the fluid chamber of the first member and to or from the fluid chamber of the second member and to or from the fluid chamber of the third member and to or from the fluid chamber of the fourth member,
   switch between distributing hydraulic fluid to or from the fluid chamber of the first member and to or from the fluid chamber of the second member and to or from the fluid chamber of the third member and to or from the fluid chamber of the fourth member and to or from the fluid chamber of the fifth member.
14. The medical device according to any one of the preceding aspects, wherein the at least one operable valve is a rotatable operable valve.
15. The medical device according to any one of aspects 13 and 14, further comprising an actuator for operating the operable valve, and wherein the actuator is at least one of: an electric actuator and a hydraulic actuator.
16. The medical device according to any one of the preceding aspects, wherein the operable valve is configured to simultaneously distribute fluid to the first fluid chamber of the first member and the second fluid chamber of the second member, for causing the first and second members to bend or pivot in different directions, away from each other, for stretching the stomach wall between the first and second member.
17. The medical device according to any one of the preceding aspects, wherein the operable valve is configured to simultaneously distribute fluid to the first fluid chamber of the second member and the second fluid chamber of the third member, for causing the second and third members to bend or pivot in different directions, away from each other, for stretching the stomach wall between the second and third member.
18. The medical device according to any one of the preceding aspects, wherein each of the first, second, third, fourth and fifth member comprises a stomach engager portion.
19. The medical device according to any one of the preceding aspects, wherein the stomach engager portion is configured to be invaginated by the stomach wall using stomach-to-stomach sutures or staplers.
20. The medical device according to any one of the preceding aspects, wherein the stomach engager portion comprises at least one of:
   a fixation portion configured for fixation of the stomach engager portion to tissue of the stomach wall using at least one of sutures and staplers, and
   a tissue growth promoting structure such that the stomach engager portion can be fixated to the stomach wall by in-growth of fibrotic tissue.
21. The medical device according to any one of the preceding aspects, wherein the main portion comprises a fluid conduit for conducting fluid to at least one of the first, second, third, fourth and fifth member.
22. The medical device according to aspect 19, further comprising an implantable hydraulic pump for pumping fluid to the fluid conduit.
23. The medical device according to aspect 22, wherein the implantable pump is configured to be positioned at a remote location in relation to the main portion.
24. The medical device according to any one of the preceding aspects, further comprising an energy storage unit for directly or indirectly energizing the medical device.
25. The medical device according to any one of the preceding aspects, further comprising a controller for controlling at least one of the pump and the operable valve.
26. The medical device according to aspect 25, wherein the controller comprises a wireless transceiver for communicating wirelessly with a source external to the body of the patient.
27. The medical device according to aspect 25, further comprising at least one sensor, and wherein the controller is configured for receiving sensor input from the at least one sensor.
28. The medical device according to aspect 27, wherein the sensor is a sensor configured to sense a physical parameter of the medical device system.
29. The medical device according to aspect 28, wherein the sensor is a sensor configured to sense at least one of:
   a temperature of the medical device system,
   a parameter related to the power consumption of the medical device system,
   a parameter related to strain in the medical device system,
   a parameter related to a status of the energy storage unit, or
   a parameter related to the wireless transfer of energy from a source external to the body of the patient.
30. The medical device according to aspect 27, wherein the sensor is a sensor configured to sense a physiological parameter of the patient.
31. The medical device according to aspect 30, wherein the sensor is a sensor configured to sense at least one of:
   a parameter related to the patient swallowing,
   a local temperature,
   a systemic temperature,
   blood saturation,
   blood oxygenation,
   blood pressure,
   a parameter related to an ischemia marker, or
   pH.
32. The medical device according to aspect 31, wherein the sensor configured to sense a parameter related to the patient swallowing comprises at least one of:
   a motility sensor,
   a acoustic sensor,
   an optical sensor, or
   a strain sensor.
33. The medical device according to aspect 31, wherein the sensor configured to sense pH is configured to sense the acidity in the stomach.
34. The medical device according to any one of aspects 25-33, wherein the controller is configured to transmit information based on sensor input to a source external to the body of the patient.

### Aspect 346—Obesity_Stretch_Version2_Cable-Arms

1. A medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
2. The medical device according to aspect 1, wherein at least the second member comprises a flexible wall portion, and wherein the first flexible cable is at least partially enclosed by the flexible wall portion.
3. The medical device according to aspect 2, wherein the flexible wall portion comprises elevated and lowered portions.
4. The medical device according to aspect 3, wherein the elevated and lowered portions are configured to enable the second member to bend or pivot when fibrotic tissue is covering the elevated and lowered portions.
5. The medical device according to any one of aspects 1-4, wherein the flexible wall portion comprises at least one pleated portion.
6. The medical device according to any one of the preceding aspects, wherein the second member comprises a pivoting element pivotally fixated to the main portion, and wherein the first flexible cable causes the pivoting element to pivot when operated for stretching the stomach wall between the first and second portions.
7. The medical device according to any one of the preceding aspects, wherein the operation device comprises a second flexible cable for pulling on the second member for displacing the second member in a direction towards the first member, and wherein the second flexible cable is fixated to the second member at the distal half thereof.
8. The medical device according to any one of the preceding aspects, wherein the first member is configured to bend or pivot in relation to the main portion, such that the first member can be displaced in a direction away from the second member by the first member bending or pivoting in relation to the main portion.
9. The medical device according to aspect 8, wherein the first flexible cable is further fixated to the first member, such that pulling on the first flexible cable causes: the second member to displace in the direction away from the first member, and the first member to displace in the direction away from the second member, for stretching the stomach wall between the first and second portions.
10. The medical device according to any one of the preceding aspects, wherein the medical device comprises at least one of:
   a third member configured to bend or pivot in relation to the main portion,
   a fourth member configured to bend or pivot in relation to the main portion, and
   a fifth member configured to bend or pivot in relation to the main portion.
11. The medical device according to aspect 10, wherein at least one of:
   a second cable is connected to the second and third members, at the distal half thereof, such that pulling on the cable causes the second member to displace in a direction away from the third member, and the third member to displace in a direction away from the second member,
   a third cable is connected to the third and fourth members, at the distal half thereof, such that pulling on the cable causes the third member to displace in a direction away from the fourth member, and the fourth member to displace in a direction away from the third member,
   a fourth cable is connected to the fourth and fifth members, at the distal half thereof, such that pulling on the cable causes the fourth member to displace in a direction away from the fifth member, and the fifth member to displace in a direction away from the fourth member,
   a fifth cable is connected to the fifth and first members, at the distal half thereof, such that pulling on the cable causes the fifth member to displace in a direction away from the first member, and the first member to displace in a direction away from the fifth member.
12. The medical device according to aspect 11, further comprising an operation device configured to pull
   on at least one of:
   the first cable,
   the second cable,
   the third cable,
   the fourth cable, and
   the fifth cable.
13. The medical device according to aspect 12, wherein the operation device is configured to at least one of:
   switch between pulling on the first cable and the second cable,
   switch between pulling on the second cable and the third cable,
   switch between pulling on the third cable and the fourth cable, and
   switch between pulling on the fourth cable and the fifth cable.
14. The medical device according to any one of aspects 12 and 13, wherein the operation device is a rotatable operation device.
15. The medical device according to aspect 14, wherein the operation device comprises at least one eccentric rotatable engagement member configured to engage at least the first flexible cable for pulling the first flexible wire.
16. The medical device according to aspect 15, wherein the eccentric rotatable engagement member is further configured to engage at least one of:
   the second flexible cable,
   the third flexible cable,
   the fourth flexible cable, and
   the fifth flexible cable.
17. The medical device according to aspect 16, wherein:
   the eccentric rotatable engagement member comprises at least one of a recess or protrusion,
   at least the first flexible cable is fixated to at least one of a recess or protrusion, and
   the engagement between the recess or protrusion of the engagement member and the recess or protrusion of the second member causes the second member to pivot in relation to the main portion for displacing the second member in the first direction relative to the first member.
18. The medical device according to any one of the preceding aspects, wherein at least the second member is configured to be operated in the first direction by the first flexible cable and in an opposite direction by an elastic element.
19. The medical device according to any one of aspects 15-18, wherein the eccentric rotatable engagement member comprises a groove encircling the center of rotation of the eccentric rotatable engagement member.
20. The medical device according to aspect 19, wherein the groove is configured to control the movement of at least the second member in the first direction and in the opposite direction.
21. The medical device according to any one of the preceding aspects, wherein the stomach engager portions are configured to be invaginated by the stomach wall using stomach-to-stomach sutures or staplers.
22. The medical device according to any one of the preceding aspects, wherein the stomach engager portions comprises at least one of:
   a fixation portion configured for fixation of the stomach engager portion to tissue of the stomach wall using at least one of sutures and staplers, and
   a tissue growth promoting structure such that the stomach engager portion can be fixated to the stomach wall by in-growth of fibrotic tissue.
23. The medical device according to any one of the preceding aspects, wherein the operation device comprises at least one electrical motor.
24. The medical device system according to aspect 23, wherein the operation device further comprises a gear system configured to reduce the speed and increase the force of the movement generated by the electrical motor.
25. The medical device according to any one of the preceding aspects, wherein the operation device is partially positioned at a remote location in the body of the patient, in relation to the main portion.
26. The medical device according to any one of the preceding aspects, further comprising an energy storage unit for directly or indirectly energizing the medical device.
27. The medical device according to any one of the preceding aspects, further comprising a controller for controlling the operation device.
28. The medical device according to aspect 27, wherein the controller comprises a wireless transceiver for communicating wirelessly with a source external to the body of the patient.
29. The medical device according to any one of aspects 27 and 28, further comprising at least one sensor, and wherein the controller is configured for receiving sensor input from the at least one sensor.
30. The medical device according to aspect 29, wherein the sensor is a sensor configured to sense a physical parameter of the medical device system.
31. The medical device according to aspect 30, wherein the sensor is a sensor configured to sense at least one of:
   a temperature of the medical device system,
   a parameter related to the power consumption of the medical device system,
   a parameter related to strain in the medical device system,
   a parameter related to a status of the energy storage unit, or
   a parameter related to the wireless transfer of energy from a source external to the body of the patient.
32. The medical device according to aspect 29, wherein the sensor is a sensor configured to sense a physiological parameter of the patient.
33. The medical device according to aspect 32, wherein the sensor is a sensor configured to sense at least one of:
   a parameter related to the patient swallowing,
   a local temperature,
   a systemic temperature,
   blood saturation,
   blood oxygenation,
   blood pressure,
   a parameter related to an ischemia marker, or
   pH.
34. The medical device according to aspect 33, wherein the sensor configured to sense a parameter related to the patient swallowing comprises at least one of:
   a motility sensor,
   a acoustic sensor,
   an optical sensor, or
   a strain sensor.
35. The medical device according to aspect 33, wherein the sensor configured to sense pH is configured to sense the acidity in the stomach.
36. The medical device according to any one of aspects 29-35, wherein the controller is configured to transmit information based on sensor input to a source external to the body of the patient.
37. The medical device according to any one if aspects 1-36, wherein the operation device comprises a memory metal or an electroactive polymer.

### Aspect 347—Obesity_Stretch_Version2_Remote-Operation

1. A medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and
   a force transferring element configured to transfer force hydraulically or mechanically from the remote unit to the main portion, for operating the second member.
2. The medical device according to aspect 1, wherein at least the second member is configured to bend or pivot in relation to the main portion, such that the second member can be displaced in a direction away from the first member.
3. The medical device according to any one of aspects 1 and 2, wherein the first member is configured to bend or pivot in relation to the main portion, such that the first member can be displaced in a direction away from the second member.
4. The medical device according to any one of the preceding aspects, further comprising a third member configured to engage a third portion of the stomach wall.
5. The medical device according to any one of the preceding aspects, further comprising a fourth member configured to engage a fourth portion of the stomach wall.
6. The medical device according to aspect 5, wherein the operation device is further configured for operating at least one of the first, third and fourth member to displace one of the first, third and fourth member relative to the main portion for stretching the stomach wall.
7. The medical device according to any one of aspects 1-6, wherein the main portion comprises at least part of the operation device.
8. The medical device according to any one of the preceding aspects, wherein at least one of the first, second, third and fourth member comprises at least part of the operation device.
9. The medical device according to any one of the preceding aspects, wherein at least one of the first, second, third and fourth member is elongated.
10. The medical device according to any one of the preceding aspects, wherein at least one of the first, second, third and fourth member is hinged for enabling pivotal movement.
11. The medical device according to any one of the preceding aspects, wherein at least one of the first, second, third and fourth member is configured to be at least partially invaginated by the tissue of the stomach wall.
12. The medical device according to any one of the preceding aspects, wherein the main portion is configured to be at least partially invaginated by the tissue of the stomach wall.
13. The medical device according to any one of the preceding aspects, wherein at least one of the first, second, third and fourth member comprises at least one fixation portion configured for fixation of the member to tissue of the stomach wall using at least one of sutures, staplers or tissue growth promoting structure.
14. The medical device according to any one of the preceding aspects, wherein the portion of the operation device placed in the remote unit comprises at least one electrical motor.
15. The medical device according to aspect 14, wherein the portion of the operation device placed in the remote unit further comprises a gear system configured to reduce the speed and increase the force of the movement generated by the electrical motor.
16. The medical device according to aspect 14, wherein a portion of the operation device is placed in the main portion, and wherein the portion of the operation device placed in the main portion comprises a receiving portion configured to receive mechanical force, and a transmission for transforming the received mechanical force into a force for displacing at least one of the members for stretching the stomach wall.
17. The medical device according to aspect 16, wherein the transmission comprises a gear system configured to reduce the speed and increase the force of the received mechanical force.
18. The medical device according to any one of aspects 16 and 17, wherein the receiving portion is configured to receive a rotating mechanical force, and wherein the transmission is configured to transform the received rotating mechanical force into a liner mechanical force.
19. The medical device according to aspect 16, wherein the receiving portion is configured to receive a linear mechanical force.
20. The medical device according to any one of aspects 1-13, wherein a portion of the operation device is placed in the main portion, and wherein the portion of the operation device placed in the main portion comprises:
   a receiving portion configured to receive hydraulic force, and
   a transmission for transforming the received hydraulic force into a force for displacing at least one of the members for stretching the stomach wall.
21. The medical device according to aspect 20, wherein the transmission comprises at least one hydraulic cylinder.
22. The medical device according to any one of the preceding aspects, wherein the portion of the operation device placed in the main portion comprises at least one operable valve.
23. The medical device according to any one of the preceding aspects, wherein the portion of the operation device placed in the remote unit comprises at least one operable valve.
24. The medical device according to any one of aspects 22 and 23, wherein the operable valve is configured to control the flow of hydraulic fluid from a first state in which the hydraulic fluid can operate the first member to a second state in which the hydraulic fluid can operate the second member.
25. The medical device according to any one of the preceding aspects, wherein the portion of the operation device placed in the remote unit comprises at least one hydraulic pump.
26. The medical device according to any one of the preceding aspects, wherein the remote unit comprises a hydraulic reservoir for holding a hydraulic fluid.
27. The medical device according to any one of the preceding aspects, wherein the remote unit comprises an injection port for at least one of injecting and removing hydraulic fluid from the medical device.
28. The medical device according to any one of the preceding aspects, wherein the remote unit further comprises an energy storage unit for directly or indirectly energizing the medical device.
28. The medical device according to any one of the preceding aspects, wherein the remote unit further comprises a controller for controlling the operation device.
29. The medical device according to aspect 28, wherein the controller comprises a wireless transceiver for communicating wirelessly with a source external to the body of the patient.
30. The medical device according to aspect 29, further comprising at least one sensor, and wherein the controller is configured for receiving sensor input from the at least one sensor.
31. The medical device according to aspect 30, wherein the sensor is a sensor configured to sense a physical parameter of the medical device system.
32. The medical device according to aspect 31, wherein the sensor is a sensor configured to
   sense at least one of:
   a temperature of the medical device system,
   a parameter related to the power consumption of the medical device system,
   a parameter related to strain in the medical device system,
   a parameter related to a status of the energy storage unit,
   a parameter related to the wireless transfer of energy from a source external to the body of the patient,
   a hydraulic pressure.
33. The medical device according to aspect 32, wherein the sensor is a sensor configured to sense a physiological parameter of the patient.
34. The medical device according to aspect 33, wherein the sensor is a sensor configured to sense at least one of:
   a parameter related to the patient swallowing,
   a local temperature,
   a systemic temperature,
   blood saturation,
   blood oxygenation,
   blood pressure,
   a parameter related to an ischemia marker, or
   pH.
35. The medical device according to aspect 34, wherein the sensor configured to sense a parameter related to the patient swallowing comprises at least one of:
   a motility sensor,
   a acoustic sensor,
   an optical sensor, or
   a strain sensor.
36. The medical device according to aspect 34, wherein the sensor configured to sense pH is configured to sense the acidity in the stomach.
37. The medical device according to any one of aspects 30-36, wherein the controller is configured to transmit information based on sensor input to a source external to the body of the patient.
38. The medical device according to aspect 37, wherein the controller is configured to transmit the information based on sensor input to a source external to the body of the patient, wirelessly.
39. The medical device according to any one of the preceding aspects, wherein the medical device further comprises at least one lead for transferring electrical energy and/or information from the remote unit to the main portion.
40. The medical device according to any one of the preceding aspects, wherein the medical device further comprises a housing configured to enclose the remote unit.
41. The medical device according to aspect 40, wherein a first portion of the housing is made from titanium and a second portion of the housing is made from a ceramic material.
42. The medical device according to aspect 41, wherein the portion of the housing made from a ceramic material comprises at least one coil embedded in the ceramic material.
43. The medical device according to aspect 41, wherein the portion of the housing made from a ceramic material comprises at least one lead embedded in the ceramic material, for transferring electrical energy and/or information through the enclosure.
44. The medical device according to any one of aspects 40-43, wherein the remote unit comprises a magnetic coupling for transferring mechanical through the housing.
45. The medical device according to any one of the preceding aspects, wherein the remote unit comprises a sensation generator adapted to generate a sensation detectable by a sense of the patient.
46. The medical device according to any one of the preceding aspects, wherein the remote unit comprises:
   a first portion configured to be placed on a first side of the tissue portion, the first portion having a first cross-sectional area in a first plane and comprises a first surface configured to engage a first tissue surface of the tissue portion,
   a second portion configured to be placed on a second side of the tissue portion, the second portion having a second cross-sectional area in a second plane and comprises a second surface configured to engage a second tissue surface of the tissue portion, and
   a connecting portion configured to be placed through a hole in the tissue portion, the connecting portion having a third cross-sectional area in a third plane and is configured to connect the first portion to the second portion, wherein:
      the first, second and third planes are parallel to each other,
      the third cross-sectional area is smaller than the first and second cross-sectional areas, such that the first and second portions are prevented from travelling through the hole in the tissue portion in a direction perpendicular to the first, second and third planes.
47. The medical device according to aspect 46, wherein the first portion comprises a first wireless energy receiver for receiving energy transmitted wirelessly from an external wireless energy transmitter, and an internal wireless energy transmitter configured to transmit energy wirelessly to the second portion, and the second portion comprises a second wireless energy receiver for receiving energy from the first portion wirelessly.
48. The medical device according to aspect 47, wherein the first portion comprises a first energy storage unit connected to the first wireless energy receiver.
49. The medical device according to any one of aspects 47 and 48, wherein the second portion comprises a second energy storage unit connected to the second wireless energy receiver.
50. The medical device according to aspect 49, wherein:
   the first wireless energy receiver is configured to receive energy from the external wireless energy transmitter and store the received energy in the first energy storage unit,
   the wireless energy transmitter is configured to transmit energy stored in the first energy storage unit to the second wireless energy receiver, and
   the second wireless energy receiver is configured to receive energy from the internal wireless energy transmitter and store the received energy in the second energy storage unit.
51. The medical device according to any one of aspects 46-50, wherein:
   the first portion comprises a first wireless communication receiver for receiving wireless communication from an external device,
   the first portion comprises a first wireless communication transmitter for transmitting wireless communication to a second wireless communication receiver in the second portion.
52. The medical device according to any one of aspects 46-51, wherein the first portion is detachably connected to at least one of the second portion and the connecting portion.
53. The medical device according to any one of aspects 46-52, wherein the connecting portion comprising a flange having a flange area being larger than the third cross-sectional area, such that the flange is hindered from travelling through the hole in the tissue portion, such that the second portion and the connecting portion can be held in position by the tissue portion of the patient also when the first portion is disconnected from the connecting portion.
54. The medical device according to any one of aspects 46-53, wherein the connecting portion comprises a conduit for transferring a fluid from the first portion to the second portion.
55. The medical device according to any one if aspects 1-54, wherein the operation device comprises a memory metal or an electroactive polymer.

### Aspect 348—Obesity_Stretch_Version2_Ring

1. A medical device for actively stretching a stomach wall of a patient for creating a sensation of satiety, the medical device being implantable and comprising a first member comprising a closed curve, the first member comprising a first portion comprising a first stomach engager configured to engage a first portion of the stomach wall and a second portion comprising a second stomach engager configured to engage a second portion of the stomach wall, wherein the first member is operable such that the first and second portion can move towards each other or away from each other for stretching a portion of the stomach wall.
2. The medical device according to aspect 1, wherein the first member is configured to encircle a portion of the stomach wall of the patient.
3. The medical device according to aspect 1, wherein the first member comprises a simple closed curve.
4. The medical device according to aspect 3, wherein the first member is ring shaped.
5. The medical device according to any one of the preceding aspects, wherein the first member is operable to assume an oval shape.
6. The medical device according to aspect 5, wherein the first member is operable to assume an elliptical shape.
7. The medical device according to any one of the preceding aspects, wherein the first and second portions moving away from each other causes a stretching of a portion of the stomach wall between the first and second portions.
8. The medical device according to any one of the preceding aspects, wherein the first member further comprises a third portion comprising a third stomach engager configured to engage a third portion of the stomach wall and a fourth portion comprising a fourth stomach engager configured to engage a fourth portion of the stomach wall, and wherein the first and second portions moving towards each other causes the third and fourth portions to move away from each other.
9. The medical device according to aspect 8, wherein the third and fourth portions moving away from each other causes a stretching of a portion of the stomach wall between the third and fourth portions.
10. The medical device according to any one of the preceding aspects, wherein the first member is configured to be operated to stretch the stomach wall between the first member and a second member fixated to the stomach wall.
11. The medical device according to aspect 10, wherein the first and second portions moving towards each causes a stretching of the stomach wall between at least one of the first and second portion, and the second member.
12. The medical device according to any one of the preceding aspects, further comprising a second member configured to act as a support against the force created by the operation of the first member, such that the stomach wall between the first and second member can be stretched.
13. The medical device according to aspect 12, wherein the support is configured to be fixated to the stomach wall of the patient.
14. The medical device according to any one of aspects 12 and 13, wherein the support comprises a closed curve.
15. The medical device according to any one of the preceding aspects, wherein the first member is mechanically operable.
16. The medical device according to aspect 15, further comprising a mechanical operation device.
17. The medical device according to aspect 16, wherein the mechanical operation device comprises at least one flexible element, directly or indirectly fixated to the first and second portion of the first member, and wherein the flexible element is configured to operate the first member for at least one of:
   moving the first portion towards the second portion, and
   moving the first portion away from the second portion.
18. The medical device according to aspect 17, wherein the mechanical operation device comprises at least one second flexible element, directly or indirectly fixated to the third and fourth portion of the first member, and wherein the flexible element is configured to operate the first member for at least one of:
   moving the third portion towards the fourth portion, and
   moving the third portion away from the fourth portion.
19. The medical device according to aspect 18, wherein the mechanical operation device comprises at least one third flexible element, directly or indirectly fixated to a fifth and sixth portion of the first member, and wherein the flexible element is configured to operate the first member for at least one of:
   moving the fifth portion towards the sixth portion, and
   moving the fifth portion away from the sixth portion.
20. The medical device according to aspect 19, wherein the mechanical operation device comprises at least one fourth flexible element, directly or indirectly fixated to a seventh and eight portion of the first member, and wherein the flexible element is configured to operate the first member for at least one of:
   moving the seventh portion towards the eight portion, and
   moving the seventh portion away from the eight portion.
21. The medical device according to any one of aspects 17-20, wherein at least one of the first and second members comprises at least one recess or channel, and wherein at least one of the flexible elements travels at least partially in the recess or channel.
22. The medical device according to any one of aspects 17-21, wherein a first portion of the flexible element is directly or indirectly fixated to the first portion of the first member, and a second portion of the flexible element is directly or indirectly fixated to the second portion of the first member, such that operation of the flexible element simultaneously operates the first and second portion of the first member.
23. The medical device according to any one of aspects 17-22, further comprising at least one sheath, and wherein at least one flexible element is configured to travel through the sheath.
24. The medical device according to aspect 23, wherein the sheath comprises elevated and lowered portions, such that the sheath can remain flexible even as fibrotic tissue overgrows the sheath.
25. The medical device according to any one of aspects 1-14, wherein the first member is hydraulically operable.
26. The medical device according to aspect 25, further comprising a hydraulic operation device.
27. The medical device according to aspect 26, wherein the hydraulic operation device comprises at least one hydraulic actuator, directly or indirectly fixated to the first and second portion of the first member, and wherein the hydraulic actuator is configured to operate the first member for at least one of:
   moving the first portion towards the second portion, and
   moving the first portion away from the second portion.
28. The medical device according to aspect 27, wherein the hydraulic operation device comprises at least one second hydraulic actuator, directly or indirectly fixated to the third and fourth portion of the first member, and wherein the hydraulic actuator is configured to operate the first member for at least one of:
   moving the third portion towards the fourth portion, and
   moving the third portion away from the fourth portion.
29. The medical device according to aspect 28, wherein the hydraulic operation device comprises at least one third hydraulic actuator, directly or indirectly fixated to a fifth and sixth portion of the first member, and wherein the hydraulic actuator is configured to operate the first member for at least one of:
   moving the fifth portion towards the sixth portion, and
   moving the fifth portion away from the sixth portion.
30. The medical device according to aspect 29, wherein the hydraulic operation device comprises at least one fourth hydraulic actuator, directly or indirectly fixated to a seventh and eight portion of the first member, and wherein the hydraulic actuator is configured to operate the first member for at least one of:
   moving the seventh portion towards the eight portion, and
   moving the seventh portion away from the eight portion.
31. The medical device according to any one of aspects 27-30, wherein the medical device comprises at least one hydraulic conduit for conducting a hydraulic fluid to at least one of the hydraulic actuators.
32. The medical device according to aspect 31, wherein at least one of the first and second members comprises at least one recess or channel, and wherein the hydraulic conduit travels at least partially in the recess or channel.
33. The medical device according to any one or aspects 25-32, wherein at least one of the first and second members comprises at least one channel for conducting a hydraulic fluid.
34. The medical device according to any one of aspects 27-33, further comprising at least one sheath, and wherein at least one hydraulic actuator is configured to travel through the sheath.
35. The medical device according to aspect 34, wherein the sheath comprises elevated and lowered portions, such that the sheath can remain flexible even as fibrotic tissue overgrows the sheath.
36. The medical device according to any one of the preceding aspects, wherein the first member is flexible.
37. The medical device according to aspect 36, wherein the first member is elastic.
38. The medical device according to any one of the preceding aspects, wherein the first member is more flexible than the second member.
39. The medical device according to aspect 38, wherein the first member is more elastic than the second member.
40. The medical device according to any one of aspects 12-39, wherein the second member is substantially rigid.
41. The medical device according to any one of aspects 12-40, wherein a major portion of the second member is made from a material having a modulus of elasticity in the range 0.2 GPa-1000 GPa or in the range 1 GPa-400 GPa.
42. The medical device according to any one of aspects 12-41, wherein the second member has a modulus of elasticity in the range 0.2 GPa-1000 GPa or in the range 1 GPa-400 GPa.
43. The medical device according to any one of the preceding aspects, wherein at least one of the first and second member is configured to be invaginated by the stomach wall using stomach-to-stomach sutures or staplers.
44. The medical device according to any one of the preceding aspects, wherein at least one of the stomach engagers comprises at least one of:
   a fixation portion configured for fixation of the stomach engager to tissue of the stomach wall using at least one of sutures and staplers, and
   a tissue growth promoting structure such that the stomach engager can be fixated to the stomach wall by in-growth of fibrotic tissue.
44. The medical device according to any one of aspects 16-44, wherein a portion of the mechanical or hydraulic operation device is placed in a remote unit configured to be placed at a remote location in the body of the patient, and wherein the medical device further comprises a force transferring element configured to transfer force hydraulically or mechanically from the remote unit to the first member, for operating the first member.
45. The medical device according to aspect 44, wherein the portion of the operation device placed in the remote unit comprises at least one electrical motor.
46. The medical device according to aspect 45, wherein the portion of the operation device placed in the remote unit further comprises a gear system configured to reduce the speed and increase the force of the movement generated by the electrical motor.
47. The medical device according to any one of aspects 44-46, wherein the portion of the operation device placed in the remote unit comprises at least one pulley for propelling the flexible element.
48. The medical device according to any one of aspects 44-47, wherein the portion of the operation device placed in the remote unit comprises at least one hydraulic pump.
49. The medical device according to any one of aspects 44-48, wherein the remote unit comprises a hydraulic reservoir for holding a hydraulic fluid.
50. The medical device according to any one of aspects 44-49, wherein the remote unit comprises an injection port for at least one of injecting and removing hydraulic fluid from the medical device.
51. The medical device according to any one of aspects 44-50, wherein the remote unit further comprises an energy storage unit for directly or indirectly energizing the medical device.
52. The medical device according to any one of aspects 44-51, wherein the remote unit further comprises a controller for controlling the operation device.
53. The medical device according to aspect 52, wherein the controller comprises a wireless transceiver for communicating wirelessly with a source external to the body of the patient.
54. The medical device according to any one of the preceding aspects, further comprising at least one sensor configured to sense a physical parameter of the medical device system.
55. The medical device according to aspect 54, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect 349—Obesity-Stretch-Version2-Sensor-Acoustic

1. A medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
2. The medical device according to aspect 1, wherein the acoustic sensor comprises at least one microphone.
3. The medical device according to any one of aspects 1 and 2, wherein the acoustic sensor comprises an implantable acoustic sensor.
4. The medical device according to any one of aspects 1 and 2, wherein the controller is configured to receive an acoustic sensor signal from an acoustic sensor being placed external to the body of the patient.
5. The medical device according to any one of the preceding aspects, wherein the sensor is further configured to sense at least one parameter related to a functional status of the medical device.
6. The medical device according to aspect 5, wherein the sensor is configured to sense at least one parameter related to the operation of the operation device.
7. The medical device according to aspect 6, wherein the operation device comprises an electrical motor, and wherein the sensor is configured to sense at least one parameter related to the operation of the electrical motor.
8. The medical device according to any one of the preceding aspects, wherein the controller comprises at least one unit having a sleep mode and an active mode, and wherein the unit consumes less energy in the sleep mode than in the active mode, and wherein the unit is configured to switch from the sleep mode to the active mode on the basis of at least one signal from the acoustic sensor.
9. The medical device according to aspect 8, wherein the unit is configured to switch from the sleep mode to the active mode on the basis of a signal from the acoustic sensor related to the patient swallowing a number of times.
10. The medical device according to aspect 9, wherein the unit is configured to switch from the sleep mode to the active mode on the basis of a signal from the acoustic sensor related to the patient swallowing a number of times during a time period.
11. The medical device according to any one of the preceding aspects, wherein the controller comprises at least one filtering unit configured to filter acoustic signals related to at least one of: speech, the swallowing of saliva and chewing.
12. A method of detecting that a patient swallows, comprising receiving a wireless signal from an implanted acoustic sensor configured to sense at least one sound related to the patient swallowing.
13. The method according to aspect 12, wherein the controller comprises at least one unit having a sleep mode and an active mode, and wherein the unit consumes less energy in the sleep mode than in the active mode, and wherein unit is configured to switch from the sleep mode to the active mode on the basis of a signal from the acoustic sensor related to the patient swallowing a number of times, and wherein the method comprises the step of counting the number of times that the patient swallows.
14. The method according to aspect 12, wherein the method further comprises filtering acoustic signals related to at least one of: speech, the swallowing of saliva and chewing in a filtering unit of the controller.
15. A method of implanting s medical device for actively stretching a stomach wall of a patient for creating a sensation of satiety, the method comprising:
   fixating the medical device to the stomach wall of the patient, and
   implanting at least one acoustic sensor configured to sense at least one sound related to the patient swallowing.
16. The medical device according to any one if aspects 1-15, wherein the operation device comprises a memory metal or an electroactive polymer.

### Aspect 350—Obesity_Stretch_Version2_Sensor_Sternum

1. A medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
2. The medical device according to aspect 1, wherein the sensor comprises at least one acoustic sensor.
3. The medical device according to aspect 2, wherein the acoustic sensor comprises at least one microphone.
4. The medical device according to any one of the preceding aspects, wherein the sensor comprises at least one accelerometer.
5. The medical device according to any one of the preceding aspects, wherein the sensor comprises at least one piezo-electric or piezo-resistive sensor.
6. The medical device according to any one of the preceding aspects, wherein the sensor comprises a strain gauge.
7. The medical device according to any one of the preceding aspects, wherein the sensor is configured to be fixated to at least one of:
   a sternum, and
   at least one rib of the patient.
8. The medical device according to aspect 7, wherein the sensor is configured to be fixated to the sternum or the at least one rib of the patient is configured to be fixated to the sternum or the at least one rib of the patient, on the anterior side thereof.
9. The medical device according to aspect 7, wherein the sensor is configured to be fixated to the sternum or the at least one rib of the patient is configured to be fixated to the sternum or the at least one rib of the patient, on the posterior side thereof.
10. The medical device according to any one of aspects 1-9, wherein the sensor comprises an implantable sensor.
11. The medical device according to any one of aspects 1-8, wherein the sensor is configured to be fixated to the structure of the body comprising bone, on the outside of the skin of the patient.
12. The medical device according to aspect 11, wherein the controller is configured to receive a sensor signal from a sensor being placed external to the body of the patient.
13. The medical device according to any one of the preceding aspects, wherein the sensor is further configured to sense at least one parameter related to a functional status of the medical device.
14. The medical device according to aspect 13, wherein the sensor is configured to sense at least one parameter related to the operation of the operation device.
15. The medical device according to any one of the preceding aspects, wherein the controller comprises at least one unit having a sleep mode and an active mode, and wherein the unit consumes less energy in the sleep mode than in the active mode, and wherein the unit is configured to switch from the sleep mode to the active mode on the basis of at least one signal from the sensor.
16. The medical device according to aspect 15, wherein the unit is configured to switch from the sleep mode to the active mode on the basis of a signal from the sensor related to the patient swallowing a number of times.
17. The medical device according to aspect 16, wherein the unit is configured to switch from the sleep mode to the active mode on the basis of a signal from the sensor related to the patient swallowing a number of times during a time period.
18. The medical device according to any one of the preceding aspects, wherein the controller comprises at least one filtering unit configured to filter signals related to at least one of: speech, the swallowing of saliva and chewing.
19. The medical device according to any one of the preceding aspects, wherein the controller comprises at least one filtering unit configured to filter signals related to movement of the patient.
20. The medical device according to aspect 4, wherein the accelerometer is configured to measure acceleration in one plane.
21. The medical device according to aspect 20, wherein the accelerometer is configured to measure acceleration in two planes.
22. The medical device according to aspect 20, wherein the accelerometer is configured to measure acceleration in two planes.
23. The medical device according to any one of the preceding aspects, wherein the sensor signals are configured to be sent outside the body of the patient.
24. The medical device according to aspect 23, wherein the sensor signals are configured to be sent to a filtering unit positioned on the outside the body of the patient.
25. A method of detecting that a patient swallows, comprising receiving a wireless signal from an implanted sensor fixated to a structure of the body comprising bone configured to sense at least one sound related to the patient swallowing.
25. A method of detecting that a patient swallows, comprising receiving a signal from a sensor fixated to the body of the patient and being configured to sense at least one of a sound, a movement and a vibration related to the patient swallowing.
26. A method of implanting s medical device for actively stretching a stomach wall of a patient for creating a sensation of satiety, the method comprising:
   fixating the medical device to the stomach wall of the patient, and
   fixating at least one sensor configured to sense at least one parameter related to the patient swallowing to a structure of the body comprising bone.
27. The medical device according to any one if aspects 1-26, wherein the operation device comprises a memory metal or an electroactive polymer.

### Aspect 351—Obesity_Stretch_Version2_Switching-Actuator

1. A medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
2. The medical device according to aspect 1, wherein the actuator is configured to:
   in a first state, operate the first member for displacing the first stomach engager of the first member in away from the second stomach engager of the first member, for stretching a stomach wall portion between the first and second stomach engagers, and
   in a second state, operate the second member for displacing the first stomach engager of the second member in away from the second stomach engager of the second member, for stretching a stomach wall portion between the first and second stomach engagers.
3. The medical device according to aspect 1, wherein the actuator is configured to:
   in a first state, operate the first member for displacing the first stomach engager of the first member towards the second stomach engager of the first member, for at least stretching a stomach wall portion between the first and second members, and
   in a second state, operate the second member for displacing the first stomach engager of the second member towards the second stomach engager of the second member, for at least stretching a stomach wall portion between the first and second members.
4. The medical device according to any one of the preceding aspects, wherein the first and second stomach engagers are pivotably fixated to the main portion.
5. The medical device according to aspect 4, wherein the first and second stomach engagers of the first member are pivotably fixated to the main portion at a common point of pivot.
6. The medical device according to aspect 5, wherein the first member is scissor-like and the first and second stomach engagers of the first member are operable in a scissors-like manner, such that the first and second stomach engagers can be displaced towards each other and away from each other by the operation of the handle-like portion of the scissor-like first member.
7. The medical device according to any one of the preceding aspects, wherein the operation device is configured to displace the actuator by a rotational movement.
8. The medical device according to aspect 7, wherein the operation device is configured to displace the actuator by a combination of linear and rotational movement.
9. The medical device according to any one of the preceding aspects, wherein the actuator in a first state is engaged with the first member and in a second state is engaged with the second member.
10. The medical device according to aspect 9, wherein the operation device is configured to disengage the actuator from the first member by linear displacement of the actuator.
11. The medical device according to any one of the preceding aspects, further comprising a third member comprising:
   a first stomach engager configured to engage a fifth portion of the stomach wall, and
   a second stomach engager portion configured to engage a sixth portion of the stomach wall, and wherein
the operation device is configured to displace the actuator, such that the actuator is configured to, in a third state, operate the third member for displacing the first stomach engager of the third member in relation to the second stomach engager of the third member, for stretching the stomach wall.
12. The medical device according to aspect 11, further comprising a fourth member comprising:
   a first stomach engager configured to engage a seventh portion of the stomach wall, and
   a second stomach engager portion configured to engage an eight portion of the stomach wall, and wherein the operation device is configured to displace the actuator, such that the actuator is configured to, in a fourth state, operate the fourth member for displacing the first stomach engager of the fourth member in relation to the second stomach engager of the fourth member, for stretching the stomach wall.
13. The medical device according to any one of the preceding aspects, wherein the operation device comprises a hydraulic operation device and the actuator comprises a hydraulic actuator.
14. The medical device according to aspect 13, wherein the hydraulic actuator comprises at least one hydraulic cylinder.
15. The medical device according to any one of aspects 13 and 14, wherein the hydraulic actuator comprises at least one flexible member comprising elevated and lowered portions.
16. The medical device according to aspect 15, wherein the flexible member comprises at least one bellows.
17. The medical device according to any one of aspects 15 and 16, wherein the elevated and lowered portions are configured to enable the hydraulic actuator to be operable when fibrotic tissue is covering the elevated and lowered portions.
18. The medical device according to any one of aspects 15-17, wherein the operation device comprises an implantable pump configured to at least one of:
   provide hydraulic fluid for displacing the actuator by a rotational movement,
   provide hydraulic fluid for displacing the actuator by a linear movement, and
   provide hydraulic fluid for operating the actuator for displacing the first stomach engager in relation to the second stomach engager.
19. The medical device according to aspect 18, wherein the implantable pump is configured to be positioned at a remote location in relation to the main portion.
20. The medical device according to any one of aspects 15-19, wherein the operation device operable by pressurized hydraulic fluid in a first direction and by an elastic element in an opposite direction.
21. The medical device according to aspect 20, wherein the actuator is operable by pressurized hydraulic fluid in a first direction and by an elastic element in an opposite direction.
22. The medical device according to any one of aspects 18-21, wherein the operation device further comprises a valve connected to the implantable pump, and wherein the valve is configured to:
   in a first state direct hydraulic fluid for displacing the actuator, and
   in a second state direct hydraulic fluid for operating the actuator for displacing the first stomach engager in relation to the second stomach engager.
23. The medical device according to any one of aspects 1-12, wherein the operation device comprises a mechanical operation device and the actuator comprises a mechanical actuator.
24. The medical device according to aspect 23, wherein the mechanical actuator comprises at least one electrical motor or solenoid.
25. The medical device according to aspect 24, wherein the at least one electrical motor or solenoid is configured to be positioned at a remote location in relation to the main portion.
26. The medical device according to any one of aspects 23-25, wherein the mechanical actuator comprises at least one flexible member comprising elevated and lowered portions.
27. The medical device according to aspect 26, wherein the elevated and lowered portions are configured to enable the mechanical actuator to be operable when fibrotic tissue is covering the elevated and lowered portions.
28. The medical device according to any one of aspects 25-27, wherein the electrical motor or solenoid is configured to at least one of:
   displace the actuator by a rotational movement,
   displace the actuator by a linear movement, and
   operate the actuator for displacing the first stomach engager in relation to the second stomach engager.
29. The medical device according to any one of the preceding aspects, wherein the stomach engagers are configured to be invaginated by the stomach wall using stomach-to-stomach sutures or staplers.
30. The medical device according to any one of the preceding aspects, wherein the stomach engagers comprises at least one of:
   a fixation portion configured for fixation of the stomach engagers to tissue of the stomach wall using at least one of sutures and staplers, and
   a tissue growth promoting structure such that the stomach engagers can be fixated to the stomach wall by in-growth of fibrotic tissue.
31. The medical device according to any one of the preceding aspects, further comprising an energy storage unit for directly or indirectly energizing the medical device.
32. The medical device according to any one of the preceding aspects, further comprising a controller for controlling the operation device.
33. The medical device according to aspect 32, wherein the controller comprises a wireless transceiver for communicating wirelessly with a source external to the body of the patient.
34. The medical device according to any one of aspects 32 and 33, further comprising at least one sensor, and wherein the controller is configured for receiving sensor input from the at least one sensor.
35. The medical device according to aspect 34, wherein the sensor is a sensor configured to sense a physical parameter of the medical device system.
36. The medical device according to aspect 35, wherein the sensor is a sensor configured to sense at least one of:
   a temperature of the medical device system,
   a parameter related to the power consumption of the medical device system,
   a parameter related to strain in the medical device system,
   a parameter related to a status of the energy storage unit, or
   a parameter related to the wireless transfer of energy from a source external to the body of the patient.
37. The medical device according to aspect 34, wherein the sensor is a sensor configured to sense a physiological parameter of the patient.
38. The medical device according to aspect 37, wherein the sensor is a sensor configured to sense at least one of:
   a parameter related to the patient swallowing,
   a local temperature,
   a systemic temperature,
   blood saturation,
   blood oxygenation,
   blood pressure,
   a parameter related to an ischemia marker, or
   pH.
39. The medical device according to aspect 38, wherein the sensor configured to sense a parameter related to the patient swallowing comprises at least one of:
   a motility sensor,
   a acoustic sensor,
   an optical sensor, or
   a strain sensor.
40. The medical device according to aspect 38, wherein the sensor configured to sense pH is configured to sense the acidity in the stomach.
41. The medical device according to any one of aspects 32-40, wherein the controller is configured to transmit information based on sensor input to a source external to the body of the patient.
42. The medical device according to any one if aspects 1-41, wherein the operation device comprises a memory metal or an electroactive polymer.

### Aspect 378—Subcutaneous_Control_Pop-Rivet2_Outside-Peritoneum

1. A method of implanting a medical device for stretching the stomach wall, the method comprising:
   placing a first portion of a remote unit between the skin of the patient and a layer of muscular tissue of the abdominal wall,
   placing a second portion of the remote unit between a peritoneum and a layer of muscular tissue of the abdominal wall, wherein the first and second portions are configured to be connected by a connecting portion extending through at least one layer of muscular tissue of the abdominal wall,
   placing a body engaging portion of the powered medical device in connection with a tissue or an organ of the patient which is to be affected by the powered medical device, and
   placing a transferring member, configured to transfer at least one of energy and force from the second portion to the body engaging portion, at least partially between a peritoneum and a layer of muscular tissue of the abdominal wall, such that at least ⅓ of the length of the transferring member is placed on the outside of the peritoneum.
2. The method according to aspect 1, wherein the transferring member is configured to transfer mechanical force from the second portion to the body engaging portion.
3. The method according to aspect 1, wherein the transferring member is configured to transfer hydraulic force from the second portion to the body engaging portion.
4. The method according to any one of aspects 1-3, wherein the transferring member is configured to transfer electrical energy force from the second portion to the body engaging portion.
5. The method according to any one of the preceding aspects, wherein the transferring member is configured to transfer data between the second portion and the body engaging portion.
6. The method according to any one of the preceding aspects, wherein the step of placing the transferring member comprises placing the transferring member at least partially between the peritoneum and the layer of muscular tissue of the abdominal wall, such that at least ½ of the length of the transferring member is placed on the outside of the peritoneum of the patient.
7. The method according to any one of the preceding aspects, wherein the step of placing the transferring member comprises placing the transferring member at least partially between the peritoneum and the layer of muscular tissue of the abdominal wall, such that at least ⅔ of the length of the transferring member is placed on the outside of the peritoneum of the patient.
8. The method according to any one of the preceding aspects, wherein the step of placing the transferring member comprises placing the transferring member entirely outside of the peritoneum of the patient.
10. The method according to any one of the preceding aspects, wherein the step of placing the transferring member comprises placing the transferring member such that it extends from the second portion to an area between the rib cage and the peritoneum of the patient, outside of the peritoneum.
11. The method according to any one of the preceding aspects, wherein the step of placing the transferring member comprises placing the transferring member such that it extends from the second portion to an area between the stomach and the thoracic diaphragm of the patient.
12. The method according to any one of the preceding aspects, wherein the step of placing the transferring member comprises placing the transferring member such that it extends from the second portion to the stomach of the patient.
13. The method according to any one of the preceding aspects, wherein the step of placing the second portion of the remote unit between the peritoneum and the layer of muscular tissue of the abdominal wall comprises placing the second portion between a first and second layer of muscular tissue of the abdominal wall.
14. The method according to any one of the preceding aspects, wherein the step of placing the second portion comprises placing a second portion comprising an electrical motor.
15. The method according to any one of the preceding aspects, wherein the step of placing the second portion comprises placing a second portion comprising a hydraulic pump.
16. The method according to any one of the preceding aspects, wherein the step of placing the second portion comprises placing a second portion comprising an energy storage unit.
17. The method according to any one of the preceding aspects, wherein the step of placing the second portion comprises placing a second portion comprising a receiver for receiving at least one of: energy and communication, wirelessly.
18. The method according to any one of the preceding aspects, wherein the step of placing the first portion comprises placing a first portion comprising a transmitter for transmitting at least one of: energy and communication, wirelessly.
19. The method according to any one of the preceding aspects, wherein the step of placing the second portion comprises placing a second portion comprising a controller involved in the control of the powered medical device.
20. The method according to any one of the preceding aspects, wherein the second portion is elongated and has a length axis extending substantially in the direction of the elongation of the second portion, and wherein the step of placing the second portion comprises placing the second portion such that the length axis is substantially parallel with the cranial-caudal axis of the patient.
21. The method according to any one of aspects 1-19, wherein the second portion is elongated and has a length axis extending substantially in the direction of the elongation of the second portion, and wherein the step of placing the first portion comprises placing the second portion such that the length axis is substantially perpendicular with the cranial-caudal axis of the patient.
22. The method according to any one of the preceding aspects, wherein the second portion is elongated and has a length axis extending substantially in the direction of the elongation of the second portion, and wherein the step of placing the second portion comprises entering a hole in a layer of muscular tissue of the stomach wall in the direction of the length axis of the second portion and pivoting or angling the second portion after the hole has been entered.
23. The method according to any one of the preceding aspects, wherein the step of placing the first portion of the remote unit between the skin of the patient and a layer of muscular tissue of the abdominal wall comprises placing the first portion in the subcutaneous tissue.
24. The method according to any one of aspects 1-22, wherein the step of placing the first portion of the remote unit between the skin of the patient and a layer of muscular tissue of the abdominal wall comprises placing the first portion between a first and second layer of muscular tissue of the abdominal wall.
25. The method according to any one of the preceding aspects, wherein the step of placing the first portion comprises placing a first portion comprising an energy storage unit.
26. The method according to any one of the preceding aspects, wherein the step of placing the first portion comprises placing a first portion comprising a receiver for receiving at least one of: energy and communication, wirelessly.
27. The method according to any one of the preceding aspects, wherein the step of placing the first portion comprises placing a first portion comprising a transmitter for transmitting at least one of: energy and communication, wirelessly.
28. The method according to any one of the preceding aspects, wherein the step of placing the first portion comprises placing a first portion comprising a controller involved in the control of the powered medical device.
29. The method according to any one of the preceding aspects, wherein the first portion is elongated and has a length axis extending substantially in the direction of the elongation of the first portion, and wherein the step of placing the first portion comprises placing the first portion such that the length axis is substantially parallel with the cranial-caudal axis of the patient.
30. The method according to any one of the preceding aspects, wherein the second portion is elongated and has a length axis extending substantially in the direction of the elongation of the first portion, and wherein the step of placing the first portion comprises placing the first portion such that the length axis is substantially perpendicular with the cranial-caudal axis of the patient.
31. The method according to any one of the preceding aspects, wherein the second portion is elongated and has a second portion length axis extending substantially in the direction of the elongation of the second portion second portion, and the first portion is elongated and has a first portion length axis extending substantially in the direction of the elongation of the first portion, and wherein the step of placing the first and second portions comprises placing the first and second portions such that the second portion length axis and the first portion length axis are places at an angle in relation to each other exceeding 30°.
32. The method according to aspect 38, wherein the step of placing the first and second portions comprises placing the first and second portions such that the first portion length axis and the second portion length axis are places at an angle in relation to each other exceeding 45°.
33. The method according to any one of the preceding aspects, further comprising the step of placing the connecting portion through at least one layer of muscular tissue of the abdominal wall.
34. The method according to any one of the preceding aspects, wherein the first portion, the second portion and the connecting portion are portions of a single unit.
35. The method according to any one of aspects 1-40, further comprising the step of connecting the first portion to the connecting portion, in situ.
36. The method according to any one of aspects 1-40, further comprising the step of connecting the second portion to the connecting portion, in situ.
37. The method according to any one of the preceding aspects, further comprising the step of connecting the transferring member to the second portion.

### Aspect 368—Operation_General_Bowden_Pre-Tension

1. An implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
2. The implantable operation device according to aspect 1, wherein the electrical machine comprises an electrical motor or solenoid.
3. The implantable operation device according to any one of the preceding aspects, wherein the pre-tensioning device comprises a flexible element.
4. The implantable operation device according to aspect 3, wherein the flexible element comprises at least one of a spring and an elastic material.
5. The implantable operation device according to any one of the preceding aspects, wherein the flexible shaft is configured for transferring a linear force.
6. The implantable operation device according to any one of the preceding aspects, wherein the flexible shaft is a Bowden cable.
7. The implantable operation device according to any one of the preceding aspects, further comprising a transmission for transforming the mechanical force created by electrical transforming device from a rotating mechanical force to a linear mechanical force.
8. The implantable operation device according to any one of the preceding aspects, further comprising a transmission for transforming the mechanical force created by electrical transforming device from a force having a first speed and a first strength to a force having a second lower speed and a second higher strength.
9. An implantable system comprising the implantable operation device according to any one of aspects 1-8 and a body engaging portion configured to exert a force on a body portion of the patient.
10. The implantable system according to aspect 9, wherein the body engaging portion is a constriction device configured to constrict a luminary organ of a patient.
11. The implantable device according to aspect 10, wherein the body engaging portion comprises an implantable constriction device.
12. The implantable device according to aspect 11, wherein the implantable constriction device comprises an implantable constriction device for constricting a luminary organ of the patient.
13. The implantable device according to aspect 12, wherein the implantable constriction device comprises an implantable constriction device for constricting an intestine of the patient.
14. The implantable device according to aspect 13, wherein the implantable constriction device comprises an implantable constriction device for constricting a colon or rectum of the patient.
15. The implantable device according to aspect 13, wherein the implantable constriction device comprises an implantable constriction device for constricting the intestine at a region of a stoma of the patient.
16. The implantable device according to aspect 12, wherein the implantable constriction device comprises an implantable constriction device for constricting a blood vessel of the patient.
17. The implantable device according to aspect 16, wherein the implantable constriction device for constricting a blood vessel of the patient is configured to constrict the venous blood flow leading from an erectile tissue for promoting the engorgement of the erectile tissue.
18. The implantable device according to aspect 12, wherein the implantable constriction device comprises an implantable constriction device for constricting a vas deference of the patient.
19. The implantable device according to aspect 10, wherein the body engaging portion comprises an implantable element for actively emptying the urinary bladder of the patient.
20. The implantable device according to aspect 19, wherein the implantable element for actively emptying the urinary bladder of the patient is configured to empty the bladder of the patient by compressing the urinary bladder from the outside thereof.
21. The implantable device according to aspect 20, wherein the body engaging portion comprises an implantable element for actively stretching a stomach wall of the patient to create a feeling of satiety.
22. The implantable device according to any one if aspects 1-21, wherein the operation device comprises a memory metal or an electroactive polymer.

### Aspect 369—Operation—General—Bowden-Transmission

1. An implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
2. The implantable operation device according to aspect 1, wherein the transmission comprises a gun tackle.
3. The implantable operation device according to aspect 1, wherein the transmission comprises a luff or watch tackle.
4. The implantable operation device according to aspect 1, wherein the transmission comprises a double tackle.
5. The implantable operation device according to aspect 1, wherein the transmission comprises a gyn tackle.
6. The implantable operation device according to any one of the preceding aspects, further comprising a third pulley, wherein the third pully is connected to the electrical machine and wherein the flexible element is configured to be placed around the third pulley such that operation of the electrical machine pulls the flexible element.
7. The implantable operation device according to any one of the preceding aspects, wherein the flexible element is, or transitions into, a flexible shaft for transferring the mechanical force to a body engaging portion.
8. The implantable operation device according to aspect 7, wherein the flexible shaft is configured for transferring a linear force.
9. The implantable operation device according to aspect 8, wherein the flexible shaft is a Bowden cable.
10. The implantable operation device according to any one of the preceding aspects, further comprising a pre-tensioning device for creating a pre-tension in the flexible element.
11. The implantable operation device according to aspect 10, wherein the pre-tensioning device comprises a flexible element.
12. The implantable operation device according to aspect 11, wherein the flexible element comprises at least one of a spring and an elastic material.
13. The implantable operation device according to any one of the preceding aspects, wherein the electrical machine comprises an electrical motor or solenoid.
14. The implantable operation device according to any one of the preceding aspects, further comprising a transmission for transforming the mechanical force created by electrical machine from a rotating mechanical force to a linear mechanical force.
15. The implantable operation device according to any one of the preceding aspects, further comprising a transmission for transforming the mechanical force created by the electrical machine from a force having a first speed and a first strength to a force having a second lower speed and a second higher strength.
16. The implantable operation device according to any one of aspect 13-15, wherein the electrical motor is a piezoelectric motor.
17. The implantable operation device according to aspect 16, wherein the piezoelectric motor is a piezoelectric inchworm motor.
18. The implantable operation device according to aspect 16, wherein the piezoelectric motor is a piezoelectric inertial motor.
19. The implantable operation device according to aspect 16, wherein the piezoelectric motor is a piezoelectric walk-drive motor.
20. The implantable operation device according to any one of aspects 16-19, wherein the piezoelectric motor is configured to operate with at least one of:
   a rotational speed in the range 1 mrad/s-100 mrad/s, and
   a torque in the range 100 Nmm-900 Nmm.
21. The implantable operation device according to aspect 16, wherein the piezoelectric motor is a piezoelectric ultrasonic motor.
22. The implantable operation device according to aspect 21, wherein the piezoelectric ultrasonic motor is a traveling wave ultrasonic motor.
23. The implantable operation device according to aspect 21, wherein the piezoelectric ultrasonic motor is a standing wave ultrasonic motor.
24. The implantable operation device according to any one of aspects 16-23, wherein the piezoelectric motor is substantially non-magnetic.
25. The implantable operation device according to any one of aspects 16-24, wherein the piezoelectric motor is substantially non-metallic.
26. The implantable operation device according to any one of aspects 16-25, wherein the piezoelectric operation device is a reversable piezoelectric operation device.
27. An implantable system comprising the implantable operation device according to any one of aspects 1-26, and an implantable element configured to exert a force on a body portion of the patient.
28. The implantable device according to aspect 27, wherein the implantable element configured to exert a force on a body portion of the patient is an implantable constriction device.
29. The implantable system according to aspect 28, wherein the implantable constriction device comprises an implantable constriction device for constricting a luminary organ of the patient.
30. The implantable system according to aspect 29, wherein the implantable constriction device comprises an implantable constriction device for constricting an intestine of the patient.
31. The implantable system according to aspect 30, wherein the implantable constriction device comprises an implantable constriction device for constricting a colon or rectum of the patient.
32. The implantable system according to aspect 30, wherein the implantable constriction device comprises an implantable constriction device for constricting the intestine at a region of a stoma of the patient.
33. The implantable system according to aspect 28, wherein the implantable constriction device comprises an implantable constriction device for constricting a blood vessel of the patient.
34. The implantable system according to aspect 33, wherein the implantable constriction device for constricting a blood vessel of the patient is configured to constrict the venous blood flow leading from an erectile tissue for promoting the engorgement of the erectile tissue.
35. The implantable system according to aspect 28, wherein the implantable constriction device comprises an implantable constriction device for constricting a vas deference of the patient.
36. The implantable system according to aspect 27, wherein the implantable element configured to exert a force on a body portion of the patient is an implantable element for actively emptying the urinary bladder of the patient.
37. The implantable system according to aspect 26, wherein the implantable element for actively emptying the urinary bladder of the patient is configured to empty the bladder of the patient by compressing the urinary bladder from the outside thereof.
38. The implantable system according to aspect 27, wherein the implantable element configured to exert a force on a body portion of the patient is an implantable element for actively stretching a stomach wall of the patient to create a feeling of satiety.

### Aspect 348Pc—Obesity_Stretch_Version2_Moving-Point-In-Ring

1. A medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
2. The medical device according to aspect 1, wherein the second member is configured to encircle a portion of the stomach wall of the patient.
3. The medical device according to aspect 1, wherein the second member comprises a simple closed curve.
4. The medical device according to aspect 3, wherein the second member is ring shaped.
5. The medical device according to any one of aspects 1-4, wherein the first member is configured to encircle a portion of the stomach wall of the patient.
6. The medical device according to aspect 5, wherein the first member comprises a simple closed curve.
7. The medical device according to aspect 6, wherein the first member is ring shaped.
8. The medical device according to any one of the preceding aspects, wherein the second member is configured to be invaginated by the stomach wall.
9. The medical device according to any one of the preceding aspects, wherein the first member is configured to be invaginated by the stomach wall.
10. The medical device according to any one of the preceding aspects, wherein the first member is mechanically operable.
11. The medical device according to aspect 10, further comprising a mechanical operation device for operating the first member.
12. The medical device according to aspect 11, wherein the mechanical operation device comprises at least one flexible element, directly or indirectly fixated to the first member, wherein the flexible element is configured to operate the first member for moving the first member in a first direction towards the second member.
13. The medical device according to aspect 12, wherein the mechanical operation device comprises at least one second flexible element, directly or indirectly fixated to the first member, and wherein the second flexible element is configured to operate the first member for moving the first member in a second direction towards the second member, the second direction being different from the first direction.
14. The medical device according to aspect 13, wherein the mechanical operation device comprises at least one third flexible element, directly or indirectly fixated to the first member, and wherein the third flexible element is configured to operate the first member for moving the first member in a third direction towards the second member, the third direction being different from the first and second directions.
15. The medical device according to any one of aspects 12 and 13, wherein the second member comprises at least one recess or channel, and wherein at least one of the flexible elements are configured to travel at least partially in the recess or channel.
16. The medical device according to any one of aspects 12-15, further comprising at least one sheath, and wherein at least one flexible element is configured to travel through the sheath.
17. The medical device according to aspect 16, wherein the sheath comprises elevated and lowered portions, such that the sheath can remain flexible even as fibrotic tissue overgrows the sheath.
18. The medical device according to aspect 17, wherein at least a portion of the sheath is shaped like a bellows.
19. The medical device according to any one of aspects 16-18, wherein the sheath is connected to the first and second members, such that at least one flexible element can travel from the first to the second member through the sheath.
20. The medical device according to any one of aspects 12-19, wherein the mechanical operation device comprises at least one pulley, and wherein the at least one flexible element is configured to engage the pulley such that transmission is created.
21. The medical device according to aspect 20, wherein the pulley is connected to the first member and wherein:
   a first flexible element is configured to travel from a first point on the second member to the pulley such that the pulley is moved closer to the first point by the first flexible element being pulled in a direction towards the first point, and
   the transmission created by the pulley causes the pulley to move towards the first point at a lower speed than the first flexible element moves towards the first point.
22. The medical device according to aspect 21, further comprising a second pulley connected to the first member, and wherein:
   a second flexible element is configured to travel from a second point on the second member to the second pulley such that the second pulley is moved closer to the second point by the second flexible element being pulled in a direction towards the second point, and
   the transmission created by the second pulley causes the second pulley to move towards the second point at a lower speed than the second flexible element moves towards the second point.
23. The medical device according to aspect 11, wherein the mechanical operation device comprises at least one movable arm having a first portion connected to the first member, and a second portion connected to the second member.
24. The medical device according to aspect 23, wherein the at least one movable arm comprises at least one operable joint.
25. The medical device according to aspect 23, wherein the at least one movable arm is configured to move the first member relative to the second member in at least a first direction and a second direction, where the second direction is opposite the first direction.
26. The medical device according to aspect 25, wherein the at least one movable arm is further configured to move the first member relative to the second member in at least a third direction different from the first and second direction.
27. The medical device according to any one of aspects 12-26, wherein:
   a first portion of a flexible element is directly or indirectly fixated to a first portion of the first member, and
   a second portion of the flexible element is directly or indirectly fixated to a second portion of the first member.
28. The medical device according to aspect 27, wherein the flexible element is connected to a propulsion device for propelling the flexible element, and wherein:
   propulsion of the flexible element in a first direction causes the first member to move towards a first point on the second member, and
   propulsion of the flexible element in a second direction causes the first member to move towards a second point on the second member.
29. The medical device according to any one of the preceding aspects, wherein the first member is hydraulically operable.
30. The medical device according to aspect 29, further comprising a hydraulic operation device for operating the first member.
31. The medical device according to aspect 30, wherein the hydraulic operation device comprises at least one hydraulic actuator, directly or indirectly fixated to the first and second members, and wherein the hydraulic actuator is configured to operate the first member for moving the first member in a first direction towards the second member.
32. The medical device according to aspect 31, wherein the hydraulic operation device comprises at least one second hydraulic actuator, directly or indirectly fixated to the first and second members, and wherein the second hydraulic actuator is configured to operate the first member for moving the first member in a second direction towards the second member, the second direction being different from the first direction.
33. The medical device according to any one of aspects 29-32, wherein the medical device comprises at least one hydraulic conduit for conducting a hydraulic fluid to at least one of the hydraulic actuators.
34. The medical device according to aspect 33, wherein at least one of the first and second members comprises at least one recess or channel, and wherein the hydraulic conduit travels at least partially in the recess or channel.
35. The medical device according to any one or aspects 29-34, wherein at least one of the first and second members comprises at least one channel for conducting a hydraulic fluid.
36. The medical device according to any one of aspects 29-35, further comprising at least one sheath, and wherein at least one hydraulic actuator is configured to travel through the sheath.
37. The medical device according to aspect 36, wherein the sheath comprises elevated and lowered portions, such that the sheath can remain flexible even as fibrotic tissue overgrows the sheath.
38. The medical device according to any one of the preceding aspects, wherein the first member is substantially rigid.
39. The medical device according to any one of the preceding aspects, wherein a major portion of the second member has a modulus of elasticity in the range 50 GPa-400 GPa.
40. The medical device according to any one of the preceding aspects, wherein a major portion of the second member is made from a material having a modulus of elasticity in the range 50 GPa-400 GPa.
41. The medical device according to any one of aspects 9-40, wherein the medical device is configured to be completely invaginated by the stomach wall, apart from elements for transferring force or communication to or from the medical device.
42. The medical device according to any one of the preceding aspects, wherein the second member is operable to adjust at least one of the length of the closed curve and the cross-sectional area of the closed curve, for calibrating the stretching of the stomach wall.
43. The medical device according to aspect 42, wherein the first member is mechanically operable for adjusting at least one of the length of the closed curve and the cross-sectional area of the closed curve, for calibrating the stretching of the stomach wall.
44. The medical device according to aspect 42, wherein the first member comprises a rotatable threaded member for adjusting the length of the closed curve, for calibrating the stretching of the stomach wall.
45. The medical device according to aspect 42, wherein the first member is hydraulically operable for adjusting at least one of the length of the closed curve and the cross-sectional area of the closed curve, for calibrating the stretching of the stomach wall.
46. The medical device according to aspect 45, wherein the first member comprises at least one inflatable member for adjusting at least one of the length of the closed curve and the cross-sectional area of the closed curve, for calibrating the stretching of the stomach wall.
47. The medical device according to any one of aspects 42-46, wherein the first member comprises at least one portion comprising elevated and lowered areas for enabling adjustment of at least one of the length of the closed curve and the cross-sectional area of the closed curve.
48. The medical device according to aspect 47, wherein the first member comprises at least one bellows-shaped portion for enabling adjustment of at least one of the length of the closed curve and the cross-sectional area of the closed curve.
49. The medical device according to any one of aspects 12-48, wherein a portion of the mechanical or hydraulic operation device is placed in a remote unit configured to be placed at a remote location in the body of the patient, and wherein the medical device further comprises a force transferring element configured to transfer force hydraulically or mechanically from the remote unit to the first member, for operating the first member.
50. The medical device according to aspect 49, wherein the portion of the operation device placed in the remote unit comprises at least one electrical motor.
51. The medical device according to aspect 50, wherein the portion of the operation device placed in the remote unit further comprises a gear system configured to reduce the speed and increase the force of the movement generated by the electrical motor.
52. The medical device according to any one of aspects 49-51, wherein the portion of the operation device placed in the remote unit comprises at least one pulley for propelling the flexible element.
53. The medical device according to any one of aspects 49-51, wherein the portion of the operation device placed in the remote unit comprises at least one hydraulic pump.
54. The medical device according to aspect 53, wherein the remote unit comprises a hydraulic reservoir for holding a hydraulic fluid.
55. The medical device according to any one of aspects 49-54, wherein the remote unit comprises an injection port for at least one of injecting and removing hydraulic fluid from the medical device.
56. The medical device according to any one of aspects 49-55, wherein the remote unit further comprises an energy storage unit for directly or indirectly energizing the medical device.
57. The medical device according to any one of aspects 49-56, wherein the remote unit further comprises a controller for controlling the operation device.
58. The medical device according to aspect 57, wherein the controller comprises a wireless transceiver for communicating wirelessly with a source external to the body of the patient.
59. The medical device according to any one of the preceding aspects, further comprising at least one sensor configured to sense a physical parameter of the medical device system.
60. The medical device according to aspect 59, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect 440—Obesity_Stretch_Version2_Deform-to-Stretch

1. A medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
2. The medical device according to aspect 1, wherein the first member comprises at least one curvature.
3. The medical device according to aspect 2, wherein at least a portion of the first member forms a circular arc.
4. The medical device according to any one of aspects 2 and 3, wherein a portion of the first member is configured to deform, and wherein the portion of the first member configured to deform comprises at least one of the curvature and the circular arc.
5. The medical device according to any one of the preceding aspects, wherein the second member comprises at least one curvature.
6. The medical device according to aspect 5, wherein at least a portion of the second member forms a circular arc.
7. The medical device according to aspect 6, wherein the second member is ring-shaped.
8. The medical device according to aspect 7, wherein the circular are of the first member has a first radius, and the circular are of the second member has a second radius, and wherein the second radius is larger than the first radius.
9. The medical device according to any one of aspects 1-8, wherein at least one portion of the second member is fixedly fixated to the first member.
10. The medical device according to aspect 9, wherein the second member comprises a connecting portion for connecting the second member to the first member.
11. The medical device according to any one of aspects 9 and 10, wherein at least two portions of the second member is fixedly fixated to the first member.
12. The medical device according to any one of the preceding aspects, wherein the first member is fixated to the second member such that the connection with the second member provides counterforce against the force needed for the first member to deform.
13. The medical device according to any one of the preceding aspects, wherein the footprint of the first member is configured to enclose a portion of the stomach wall.
14. The medical device according to any one of the preceding aspects, wherein the footprint of the second member is configured to enclose a portion of the stomach wall.
15. The medical device according to any one of the preceding aspects, wherein the footprint of the first member is smaller than the footprint of the second member.
16. The medical device according to any one of the preceding aspects, wherein the second member is configured to encircle a portion of the stomach wall of the patient.
17. The medical device according to aspect 16, wherein the second member comprises a simple closed curve.
18. The medical device according to aspect 17, wherein the second member is ring shaped.
19. The medical device according to any one of the preceding aspects, wherein the first member is configured to encircle a portion of the stomach wall of the patient.
20. The medical device according to aspect 19, wherein the first member comprises a simple closed curve.
21. The medical device according to aspect 20, wherein the first member is ring shaped.
22. The medical device according to any one of the preceding aspects, wherein the second member is configured to be invaginated by the stomach wall.
23. The medical device according to any one of the preceding aspects, wherein the first member is configured to be invaginated by the stomach wall.
24. The medical device according to any one of the preceding aspects, wherein the first member is further configured to be operated to deform such that a second portion of the first member is moved away from a second portion of the second member, for stretching a portion of the stomach wall between the second portion of the first member and the second portion of the second member.
25. The medical device according to aspect 24, wherein the first member is further configured to be operated to deform such that a third portion of the first member is moved away from a third portion of the second member, for stretching a portion of the stomach wall between the third portion of the first member and the third portion of the second member.
26. The medical device according to aspect 25, wherein the first, second and third portions are positioned substantially equidistantly from each other.
27. The medical device according to any one of the preceding aspects, wherein the first member is mechanically operable.
28. The medical device according to aspect 27, further comprising a mechanical operation device for operating the first member.
29. The medical device according to aspect 28, wherein the mechanical operation device comprises at least one flexible element, directly or indirectly fixated to the first member, wherein the flexible element is configured to operate the first member such that the first member deforms.
30. The medical device according to aspect 29, wherein a first portion of the flexible element is directly or indirectly fixated to a first portion of the flexible element, and a second portion of the flexible element is directly or indirectly fixated to a second portion of the flexible element, and wherein the flexible element is configured to operate the first member such that the first and second portion deforms.
31. The medical device according to aspect 30, wherein the flexible element is connected to a propulsion device for propelling the flexible element, and wherein:
   propulsion of the flexible element in a first direction causes the first member to deform such that the first portion of the first member moves away from a first portion of the second member, and
   propulsion of the flexible element in a second direction causes the first member to deform such that the second portion of the first member moves away from a second portion of the second member.
32. The medical device according to aspect 29, wherein the mechanical operation device comprises at least one second flexible element, directly or indirectly fixated to a second portion of the first member, and wherein the second flexible element is configured to operate the first member to deform such that a second portion of the first member is moved away from a second portion of the second member, for stretching a portion of the stomach wall between the second portion of the first member and the second portion of the second member.
33. The medical device according to aspect 32, wherein the mechanical operation device comprises at least one third flexible element, directly or indirectly fixated to a third portion of the first member, and wherein the third flexible element is configured to operate the first member to deform such that a third portion of the first member is moved away from a third portion of the second member, for stretching a portion of the stomach wall between the third portion of the first member and the third portion of the second member.
34. The medical device according to any one of the preceding aspects, wherein the first portion is a flexible portion, and wherein the first member is configured to deform by deformation of the at least one flexible portion.
35. The medical device according to aspect 34, wherein the first flexible portion comprises at least one elastic portion.
36. The medical device according to aspect 35, wherein the elastic portion comprises at least one elastic material.
37. The medical device according to aspect 36, wherein the elastic material comprises an elastic polymer material.
38. The medical device according to aspect 37, wherein the elastic polymer material is an elastic polymer material selected from a list consisting of silicone-based materials and polyurethane-based materials.
39. The medical device according to any one of the preceding aspects, wherein the first member comprises at least one articulating joint, and wherein the first member is configured to deform by articulation of the at least one articulating joint.
40. The medical device according to any one of aspects 29-39, wherein the mechanical operation device comprises at least one pulley, and wherein the at least one flexible element is configured to engage the pulley such that transmission is created.
41. The medical device according to aspect 40, wherein: a first flexible element is configured to travel from a first point on the second member to the pulley such that the pulley is moved closer to the first point by the first flexible element being pulled in a direction towards the first point, and the transmission created by the pulley causes the pulley to move towards the first point at a lower speed than the first flexible element moves towards the first point.
42. The medical device according to any one of aspects 40-41, wherein the pulley is connected to the first member.
43. The medical device according to any one of aspects 40-42, further comprising at least one second pulley, and wherein the at least one flexible element is configured to travel from the first to the second pulley such that transmission is created.
44. The medical device according to any one of aspects 27-42, wherein the mechanical operation device comprises at least one movable arm having a first portion connected to the first member, and a second portion connected to the second member.
45. The medical device according to aspect 44, wherein the at least one movable arm comprises at least one operable joint.
46. The medical device according to any one of aspects 44-45, wherein the at least one movable arm is configured to deform the first member by moving the first portion of the first member away from the first portion of the second member.
47. The medical device according to any one of the preceding aspects, wherein the first member is hydraulically operable.
48. The medical device according to aspect 47, further comprising a hydraulic operation device for operating the first member.
49. The medical device according to aspect 48, wherein the hydraulic operation device comprises at least one hydraulic actuator, directly or indirectly fixated to the first and second members, and wherein the hydraulic actuator is configured to deform the first member by moving the first portion of the first member away from the first portion of the second member.
50. The medical device according to aspect 49, wherein the hydraulic operation device comprises at least one second hydraulic actuator, directly or indirectly fixated to the first and second members, and wherein the second hydraulic actuator is configured to deform the first member by moving a second portion of the first member away from a second portion of the second member.
51. The medical device according to any one of aspects 48-50, wherein the medical device comprises at least one hydraulic conduit for conducting a hydraulic fluid to at least one of the hydraulic actuators.
52. The medical device according to any one of aspects 29-51, wherein at least one of the first and second members comprises at least one recess or channel configured to house at least one of the flexible element and the hydraulic conduit.
53. The medical device according to any one of aspects 29-52, further comprising at least one sheath configured to house at least one of the: the flexible element, the pulley, the hydraulic conduit and the hydraulic actuator.
54. The medical device according to aspect 53, wherein the sheath comprises elevated and lowered portions, such that the sheath can remain flexible even as fibrotic tissue overgrows the sheath.
55. The medical device according to aspect 54, wherein at least a portion of the sheath is shaped like a bellows.
56. The medical device according to any one of aspects 53-55, wherein the sheath is connected to the first and second members, such that at least one of the flexible element, the hydraulic conduit, and the hydraulic actuator can travel from the first to the second member through the sheath.
57. The medical device according to any one of aspects 34-38, 40-43 and 47-52, wherein the position of the flexible first portion of the first member can be altered by the first member comprising: a first member part forming a closed curve, and an operable second member part having a rigid section and an open or flexible section, wherein: the second member part is displaceable relative to the first member part such that the position of the open or flexible section of the second member part can be altered, for altering the position of the flexible first portion configured to be operated to deform, for changing the stomach wall portion to be stretched between the flexible first portion of the first member and the first portion of the second member.
58. The medical device according to aspect 57, wherein the second member part comprises at least one curvature.
59. The medical device according to aspect 58, wherein at least a portion of the second member part forms a circular arc.
60. The medical device according to any one of aspects 57-59, wherein the first member part is ring shaped.
61. The medical device according to any one of aspects 57-60, wherein at least one portion of the first member part is fixedly fixated to the second member.
62. The medical device according to aspect 61, wherein the first member part comprises a connecting portion for connecting the first member part to the second member.
63. The medical device according to any one of aspects 57-62, wherein the second member part is rotatable in relation to the first member part.
64. The medical device according to any one of aspects 57-63, wherein the second member part is positioned at least partially inside of the first member part.
65. The medical device according to any one of aspects 57-64, wherein the second member part comprises teeth configured to be engaged by corresponding teeth of an operation device for operating the second member part.
66. The medical device according to aspect 65, wherein the operation device comprises a gear wheel or worm wheel for operating the second member part.
67. The medical device according to any one of aspects 57-66, wherein the rigid section of the second member part forms a semi-circle.
68. The medical device according to aspect 67, wherein the teeth of the second member part are positioned on the outer periphery of the rigid section for engaging the gear wheel or worm wheel of the operation device for rotating the second member part inside of the first member part for altering the position of the open or flexible section of the second member part.
69. The medical device according to any one of aspects 57-68, wherein the first member part comprises elevated and lowered portions, such that the first member part can remain flexible even as fibrotic tissue overgrows the first member part.
70. The medical device according to aspect 69, wherein at least a portion of the first member part is shaped like a bellows.
71. The medical device according to any one of the preceding aspects, wherein the second member is substantially rigid.
72. The medical device according to any one of the preceding aspects, wherein a major portion of the second member is made from a material having a modulus of elasticity in the range 50 GPa-400 GPa.
73. The medical device according to any one of the preceding aspects, wherein the medical device is configured to be completely invaginated by the stomach wall, apart from elements for transferring force or communication to or from the medical device.
74. The medical device according to any one of the preceding aspects, wherein at least one of the length and the cross-sectional area of at least one of the first and second member is adjustable for calibrating the stretching of the stomach wall.
75. The medical device according to aspect 74, wherein at least one of the length and the cross-sectional area of at least one of the first and second member is mechanically adjustable, for calibrating the stretching of the stomach wall.
76. The medical device according to aspect 75, further comprising a rotatable threaded member for adjusting the length of at least one of the first and second member, for calibrating the stretching of the stomach wall.
77. The medical device according to aspect 74, wherein at least one of the length and the cross-sectional area of at least one of the first and second member is hydraulically adjustable, for calibrating the stretching of the stomach wall.
78. The medical device according to aspect 77, wherein at least one of the first and second member comprises at least one inflatable member, for calibrating the stretching of the stomach wall.
79. The medical device according to any one of aspects 74-78, wherein at least one of the first and second member comprises at least one portion comprising elevated and lowered areas for enabling adjustment of at least one of the length and the cross-sectional area of at least one of the first and second member.
80. The medical device according to aspect 79, wherein at least one of the first and second member comprises at least one bellows-shaped portion for enabling adjustment of at least one of the length and the cross-sectional area of at least one of the first and second member.
81. The medical device according to any one of aspects 28-80, wherein a portion of the mechanical or hydraulic operation device is placed in a remote unit configured to be placed at a remote location in the body of the patient, and wherein the medical device further comprises a force transferring element configured to transfer force hydraulically or mechanically from the remote unit to the first member, for operating the first member.
82. The medical device according to aspect 81, wherein the portion of the operation device placed in the remote unit comprises at least one electrical motor.
83. The medical device according to aspect 82, wherein the portion of the operation device placed in the remote unit further comprises a gear system configured to reduce the speed and increase the force of the movement generated by the electrical motor.
84. The medical device according to any one of aspects 82-83, wherein the portion of the operation device placed in the remote unit comprises at least one pulley for propelling the flexible element.
85. The medical device according to any one of aspects 82-84, wherein the portion of the operation device placed in the remote unit comprises at least one hydraulic pump.
86. The medical device according to aspect 85, wherein the remote unit comprises a hydraulic reservoir for holding a hydraulic fluid.
87. The medical device according to any one of aspects 82-86, wherein the remote unit comprises an injection port for at least one of injecting and removing hydraulic fluid from the medical device.
88. The medical device according to any one of aspects 82-87, wherein the remote unit further comprises an energy storage unit for directly or indirectly energizing the medical device.
89. The medical device according to any one of aspects 82-88, wherein the remote unit further comprises a controller for controlling the operation device.
90. The medical device according to aspect 89, wherein the controller comprises a wireless transceiver for communicating wirelessly with a source external to the body of the patient.
91. The medical device according to any one of the preceding aspects, further comprising at least one sensor configured to sense a physical parameter of the medical device system.
92. The medical device according to aspect 91, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect 441—Obesity_Stretch_Version2_Pull-to-Stretch

1. A medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
2. The medical device according to aspect 1, wherein the medical device is configured to stretch the stomach wall in at least one main stretching direction, and wherein the operation device is configured to pull at least a portion of the first member in a pulling direction having an angle relative to the main stretching direction of less than 20°.
3. The medical device according to any one of aspects 1 and 2, wherein the operation device is configured to displace the first member relative to the second member, such that at least a portion of the first member is moved further away from at least a portion of the second member, for stretching the portion of the stomach wall between the first portion of the stomach wall and the second portion of the stomach wall, for creating the sensation of satiety.
4. The medical device according to aspect 3, wherein: the first member is elongated and comprises a first main length axis in the direction of the elongation, and the second member is elongated and comprises a second main length axis in the direction of the elongation, wherein the operation device is configured to displace the first member relative to the second member, such that an angle between the first main length axis and the second main length axis is altered.
5. The medical device according to any one of the preceding aspects, wherein the operation device is configured to deform the first member, such that at least a portion of the first member is moved further away from at least a portion of the second member, for stretching the portion of the stomach wall between the first portion of the stomach wall and the second portion of the stomach wall, for creating the sensation of satiety.
6. The medical device according to aspect 3, wherein: the first member is elongated and comprises a first main length axis in the direction of the elongation, and the second member is elongated and comprises a second main length axis in the direction of the elongation, wherein the operation device is configured to deform the first member, such that an angle between the first main length axis and the second main length axis is altered.
7. The medical device according to any one of the preceding aspects, wherein the operation device is connected to second member such that the connection with the second member provides counterforce against the force needed to pull the at least a portion of the first member in a direction away from at least one portion of the second member.
8. The medical device according to any one of the preceding aspects, wherein the operation device is further configured to pull at least a portion of the first member in a second direction away from at least one portion of the second member, for stretching a portion of the stomach wall between the first portion of the stomach wall and the second portion of the stomach wall for creating the sensation of satiety, wherein the second direction is different from the first direction.
9. The medical device according to any one of the preceding aspects, wherein the operation device comprises a first and a second pulling member, and wherein the first pulling member is configured to engage a first portion of the first member and the second pulling member is configured to engage a second portion of the first member, such that: pulling on the first pulling member increases the distance between the first portion of the first member and a first portion of the second member, and pulling on the second pulling member increases the distance between the second portion of the first member and a second portion of the second member.
10. The medical device according to aspect 9, wherein: the first pulling member is configured to displace the first portion of the first member relative to the first portion of the second member, and the second pulling member is configured to displace the second portion of the first member relative to the second portion of the second member.
11. The medical device according to any one of aspects 9 and 10, wherein: the first pulling member is configured to deform the first portion of the first member, and the second pulling member is configured to deform the second portion of the first member.
12. The medical device according to any one of the preceding aspects, wherein the first member comprises at least one curvature.
13. The medical device according to aspect 12, wherein at least a portion of the first member forms a circular arc.
14. The medical device according to any one of aspects 12 and 13, wherein a portion of the first member is configured to deform, and wherein the portion of the first member configured to deform comprises at least one of the curvature and the circular arc.
15. The medical device according to any one of the preceding aspects, wherein the second member comprises at least one curvature.
16. The medical device according to aspect 15, wherein at least a portion of the second member forms a circular arc.
17. The medical device according to aspect 16, wherein at least a portion of the second member forms a circular arc.
18. The medical device according to aspect 17, wherein the circular are of the first member has a first radius, and the circular are of the second member has a second radius, and wherein the second radius is larger than the first radius.
19. The medical device according to any one of the preceding aspects, wherein at least one portion of the second member is fixedly fixated to the first member.
20. The medical device according to aspect 19, wherein the second member comprises a connecting portion for connecting the second member to the first member.
21. The medical device according to any one of the preceding aspects, wherein at least two portions of the second member are fixedly fixated to the first member.
22. The medical device according to any one of the preceding aspects, wherein the footprint of the first member is configured to enclose a portion of the stomach wall.
23. The medical device according to any one of the preceding aspects, wherein the footprint of the second member is configured to enclose a portion of the stomach wall.
24. The medical device according to any one of the preceding aspects, wherein the footprint of the first member is smaller than the footprint of the second member.
25. The medical device according to any one of the preceding aspects, wherein the second member is configured to encircle a portion of the stomach wall of the patient.
26. The medical device according to aspect 25, wherein the second member comprises a simple closed curve.
27. The medical device according to aspect 26, wherein the second member is ring shaped.
28. The medical device according to any one of the preceding aspects, wherein the first member is configured to encircle a portion of the stomach wall of the patient.
29. The medical device according to aspect 28, wherein the first member comprises a simple closed curve.
30. The medical device according to aspect 29, wherein the first member is ring shaped.
31. The medical device according to any one of the preceding aspects, wherein the second member is configured to be invaginated by the stomach wall.
32. The medical device according to any one of the preceding aspects, wherein the first member is configured to be invaginated by the stomach wall.
33. The medical device according to any one of the preceding aspects, wherein the medical device is configured to be completely invaginated by the stomach wall, apart from elements for transferring force or communication to or from the medical device.
34. The medical device according to any one of the preceding aspects, wherein the first member is mechanically operable.
35. The medical device according to aspect 34, further comprising a mechanical operation device comprising a pulling member for operating the first member.
36. The medical device according to aspect 35, wherein the pulling member comprises at least one flexible element, directly or indirectly fixated to the first member, wherein the flexible element is configured to operate the first member by pulling at least a portion of the first member in a first direction away from at least one portion of the second member.
37. The medical device according to aspect 36, wherein a first portion of the flexible element is directly or indirectly fixated to a first portion of the flexible element, and a second portion of the flexible element is directly or indirectly fixated to a second portion of the flexible element, and wherein the flexible element is configured to operate the first member such that the first and second portions are moved in a direction away from at least one portion of the second member.
38. The medical device according to aspect 37, wherein the flexible element is connected to a propulsion device for propelling the flexible element, and wherein:
   propulsion of the flexible element in a first direction causes the first portion of the first member moves away from a first portion of the second member, and
   propulsion of the flexible element in a second direction causes the second portion of the first member moves away from a second portion of the second member.
39. The medical device according to aspect 36, wherein the mechanical operation device comprises at least one second pulling member comprising at least one second flexible element, directly or indirectly fixated to a second portion of the first member, and wherein the second flexible element is configured to operate the first member such that a second portion of the first member is moved away from a second portion of the second member, for stretching a portion of the stomach wall between the second portion of the first member and the second portion of the second member.
40. The medical device according to aspect 39, wherein the mechanical operation device comprises at least one third pulling member comprising at least one third flexible element, directly or indirectly fixated to a third portion of the first member, and wherein the third flexible element is configured to operate the first member such that a third portion of the first member is moved away from a third portion of the second member, for stretching a portion of the stomach wall between the third portion of the first member and the third portion of the second member.
41. The medical device according to any one of the preceding aspects, wherein the first member comprises at least one flexible portion, and wherein the first member is configured to deform by deformation of the at least one flexible portion.
42. The medical device according to aspect 41, wherein the first flexible portion comprises at least one elastic portion.
43. The medical device according to aspect 42, wherein the elastic portion comprises at least one elastic material.
44. The medical device according to aspect 43, wherein the elastic material comprises an elastic polymer material.
45. The medical device according to aspect 44, wherein the elastic polymer material is an elastic polymer material selected from a list consisting of silicone-based materials and polyurethane-based materials.
46. The medical device according to any one of the preceding aspects, wherein the first member comprises at least one articulating joint, and wherein the first member is configured to deform by articulation of the at least one articulating joint.
47. The medical device according to any one of aspects 36-45, wherein the mechanical operation device comprises at least one pulley, and wherein the at least one flexible element is configured to engage the pulley such that transmission is created.
48. The medical device according to aspect 47, wherein:
   a first flexible element is configured to travel from a first point on the second member to the pulley such that the pulley is moved closer to the first point by the first flexible element being pulled in a direction towards the first point, and
   the transmission created by the pulley causes the pulley to move towards the first point at a lower speed than the first flexible element moves towards the first point.
49. The medical device according to any one of aspects 47-48, wherein the pulley is connected to the first member.
50. The medical device according to any one of aspects 47-49, further comprising at least one second pulley, and wherein the at least one flexible element is configured to travel from the first to the second pulley such that transmission is created.
51. The medical device according to any one of aspects 35-50, wherein the mechanical operation device comprises at least one movable arm having a first portion connected to the first member, and a second portion connected to the second member.
52. The medical device according to aspect 51, wherein the at least one movable arm comprises at least one operable joint.
53. The medical device according to any one of aspects 51-52, wherein the at least one movable arm is configured to deform the first member by moving the first portion of the first member away from the first portion of the second member.
54. The medical device according to any one of the preceding aspects, wherein the first member is hydraulically operable.
55. The medical device according to aspect 54, further comprising a hydraulic operation device comprising a pulling member for operating the first member.
56. The medical device according to aspect 55, wherein the hydraulic operation device comprises at least one pulling member comprising at least one hydraulic actuator, directly or indirectly fixated to the first and second members, and wherein the hydraulic actuator is configured to move the first member by pulling the first portion of the first member in a first direction away from the first portion of the second member.
57. The medical device according to aspect 56, wherein the hydraulic operation device comprises at least one second pulling member comprising at least one second hydraulic actuator, directly or indirectly fixated to the first and second members, and wherein the second hydraulic actuator is configured to move the first member by moving a second portion of the first member away from a second portion of the second member.
58. The medical device according to any one of aspects 55-57, wherein the medical device comprises at least one hydraulic conduit for conducting a hydraulic fluid to at least one of the hydraulic actuators.
59. The medical device according to any one of aspects 36-58, wherein at least one of the first and second members comprises at least one recess or channel configured to house at least one of the flexible element and the hydraulic conduit.
60. The medical device according to any one of aspects 36-59, further comprising at least one sheath configured to house at least one of the: the flexible element, the pulley, the hydraulic conduit and the hydraulic actuator.
61. The medical device according to aspect 60, wherein the sheath comprises elevated and lowered portions, such that the sheath can remain flexible even as fibrotic tissue overgrows the sheath.
62. The medical device according to aspect 61, wherein at least a portion of the sheath is shaped like a bellows.
63. The medical device according to any one of aspects 60-62, wherein the sheath is connected to the first and second members, such that at least one of the flexible element, the hydraulic conduit, and the hydraulic actuator can travel from the first to the second member through the sheath.
64. The medical device according to any one of aspects 1, 3, 5, 7, 8, 12-37, 41-50, 54-56 and 58-59, wherein the position of the portion of the first member configured to be pulled can be altered by the first member comprising:
   a first member part forming a closed curve, and
   an operable second member part having a rigid section and an open or flexible section, wherein:
   the second member part is displaceable relative to the first member part such that the position of the open or flexible section of the second member part can be altered, for altering the position of the portion of the first member configured to be pulled, for changing the stomach wall portion to be stretched between the portion of the first member configured to be pulled and a portion of the second member.
65. The medical device according to aspect 64, wherein the second member part comprises at least one curvature.
66. The medical device according to aspect 65, wherein at least a portion of the second member part forms a circular arc.
67. The medical device according to any one of aspects 64-66, wherein the first member part is ring shaped.
68. The medical device according to any one of aspects 64-67, wherein at least one portion of the first member part is fixedly fixated to the second member.
69. The medical device according to aspect 68, wherein the first member part comprises a connecting portion for connecting the first member part to the second member.
70. The medical device according to any one of aspects 64-69, wherein the second member part is rotatable in relation to the first member part.
71. The medical device according to any one of aspects 64-70, wherein the second member part is positioned at least partially inside of the first member part.
72. The medical device according to any one of aspects 64-71, wherein the second member part comprises teeth configured to be engaged by corresponding teeth of an operation device for operating the second member part.
73. The medical device according to aspect 72, wherein the operation device comprises a gear wheel or worm wheel for operating the second member part.
74. The medical device according to any one of aspects 64-73, wherein the rigid section of the second member part forms a semi-circle.
75. The medical device according to aspect 74, wherein the teeth of the second member part are positioned on the outer periphery of the rigid section for engaging the gear wheel or worm wheel of the operation device for rotating the second member part inside of the first member part for altering the position of the open or flexible section of the second member part.
76. The medical device according to any one of aspects 74-75, wherein the first member part comprises elevated and lowered portions, such that the first member part can remain flexible even as fibrotic tissue overgrows the first member part.
77. The medical device according to aspect 76, wherein at least a portion of the first member part is shaped like a bellows.
78. The medical device according to any one of the preceding aspects, wherein the second member is substantially rigid.
79. The medical device according to any one of the preceding aspects, wherein a major portion of the second member is made from a material having a modulus of elasticity in the range 50 GPa-400 GPa.
80. The medical device according to any one of the preceding aspects, wherein at least one of the length and the cross-sectional area of at least one of the first and second member is adjustable for calibrating the stretching of the stomach wall.
81. The medical device according to aspect 80, wherein at least one of the length and the cross-sectional area of at least one of the first and second member is mechanically adjustable, for calibrating the stretching of the stomach wall.
82. The medical device according to aspect 81, further comprising a rotatable threaded member for adjusting the length of at least one of the first and second member, for calibrating the stretching of the stomach wall.
83. The medical device according to aspect 81, wherein at least one of the length and the cross-sectional area of at least one of the first and second member is hydraulically adjustable, for calibrating the stretching of the stomach wall.
84. The medical device according to aspect 83, wherein at least one of the first and second member comprises at least one inflatable member, for calibrating the stretching of the stomach wall.
85. The medical device according to any one of aspects 80-84, wherein at least one of the first and second member comprises at least one portion comprising elevated and lowered areas for enabling adjustment of at least one of the length and the cross-sectional area of at least one of the first and second member.
86. The medical device according to aspect 85, wherein at least one of the first and second member comprises at least one bellows-shaped portion for enabling adjustment of at least one of the length and the cross-sectional area of at least one of the first and second member.
87. The medical device according to any one of aspects 35-86, wherein a portion of the mechanical or hydraulic operation device is placed in a remote unit configured to be placed at a remote location in the body of the patient, and wherein the medical device further comprises a force transferring element configured to transfer force hydraulically or mechanically from the remote unit to the first member, for operating the first member.
88. The medical device according to aspect 87, wherein the portion of the operation device placed in the remote unit comprises at least one electrical motor.
89. The medical device according to aspect 88, wherein the portion of the operation device placed in the remote unit further comprises a gear system configured to reduce the speed and increase the force of the movement generated by the electrical motor.
90. The medical device according to any one of aspects 87-88, wherein the portion of the operation device placed in the remote unit comprises at least one pulley for propelling the flexible element.
91. The medical device according to any one of aspects 88-89, wherein the portion of the operation device placed in the remote unit comprises at least one hydraulic pump.
92. The medical device according to aspect 91, wherein the remote unit comprises a hydraulic reservoir for holding a hydraulic fluid.
93. The medical device according to any one of aspects 88-92, wherein the remote unit comprises an injection port for at least one of injecting and removing hydraulic fluid from the medical device.
94. The medical device according to any one of aspects 88-93, wherein the remote unit further comprises an energy storage unit for directly or indirectly energizing the medical device.
95. The medical device according to any one of aspects 88-94, wherein the remote unit further comprises a controller for controlling the operation device.
96. The medical device according to aspect 95, wherein the controller comprises a wireless transceiver for communicating wirelessly with a source external to the body of the patient.
97. The medical device according to any one of the preceding aspects, further comprising at least one sensor configured to sense a physical parameter of the medical device system.
98. The medical device according to aspect 97, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
99. The medical device according to any one of the preceding aspects, wherein the operation device is piezoelectric operation device.
100. The medical device according to aspect 99, wherein the piezoelectric operation device comprises a piezoelectric motor.
101. The medical device according to aspect 100, wherein the piezoelectric motor is a piezoelectric inchworm motor.
102. The medical device according to aspect 100, wherein the piezoelectric motor is a piezoelectric inertial motor.
103. The medical device according to aspect 100, wherein the piezoelectric motor is a piezoelectric walk-drive motor.
104. The medical device according to any one of aspects 99-103, wherein the piezoelectric operation device is a linear piezoelectric operation device.
105. The medical device according to aspect 104, wherein the linear piezoelectric operation device operates with at least one of:
   a speed in the range 1 mm/s to 10 mm/s,
   a stroke length in the range 4 mm-30 mm, and
   a force in the range 2N-30N.
106. The medical device according to any one of aspects 100-103, wherein the piezoelectric motor is a rotational piezoelectric motor.
107. The medical device according to aspect 106, wherein the rotational piezoelectric motor is configured to operate with at least one of:
   a rotational speed in the range 1 mrad/s-100 mrad/s, and
   a torque in the range 100 Nmm-900 Nmm.
108. The medical device according to aspect 100, wherein the piezoelectric motor is a piezoelectric ultrasonic motor.
109. The medical device according to aspect 108, wherein the piezoelectric ultrasonic motor is a traveling wave ultrasonic motor.
110. The medical device according to aspect 108, wherein the piezoelectric ultrasonic motor is a standing wave ultrasonic motor.
111. The medical device according to any one of aspects 108-110, wherein the piezoelectric ultrasonic motor is a rotational piezoelectric ultrasonic motor configured to operate with at least one of:
   a rotational speed in the range 10 mrad/s-10000 mrad/s, and
   a torque in the range 20 Nmm-450 Nmm.
112. The medical device according to any one of aspects 108-110, wherein the piezoelectric ultrasonic motor is a linear piezoelectric ultrasonic motor configured to operate with at least one of:
   a speed in the range 4 mm/s-10 mm/s, and
   a force in the range 0,5N-30N.
113. The medical device according to any one of aspects 99-112, wherein the piezoelectric operation device comprises at least one bimorph piezoelectric operation device.
114. The medical device according to any one of aspects 99-113, wherein the piezoelectric operation device is substantially non-magnetic.
115. The medical device according to any one of aspects 99-114, wherein the piezoelectric operation device is substantially non-metallic.
116. The medical device according to any one of aspects 99-115, wherein the piezoelectric operation device is a reversable piezoelectric operation device.

### Aspect 442—Obesity_Stretch_Version2_Remote-Mechanical

1. A medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
2. The medical device according to aspect 1, wherein the medical device is configured to stretch the stomach wall in at least one main stretching direction, and wherein the operation device is configured to pull at least a portion of the first member in a pulling direction having an angle relative to the main stretching direction of less than 20°.
3. The medical device according to any one of aspects 1 and 2, wherein the operation device is configured to displace the first member relative to the second member, such that at least a portion of the first member is moved further away from at least a portion of the second member, for stretching the portion of the stomach wall between the first portion of the stomach wall and the second portion of the stomach wall, for creating the sensation of satiety.
4. The medical device according to aspect 3, wherein:
   the first member is elongated and comprises a first main length axis in the direction of the elongation, and
   the second member is elongated and comprises a second main length axis in the direction of the elongation, wherein
the operation device is configured to displace the first member relative to the second member, such that an angle between the first main length axis and the second main length axis is altered.
5. The medical device according to any one of the preceding aspects, wherein the operation device is configured to deform the first member, such that at least a portion of the first member is moved further away from at least a portion of the second member, for stretching the portion of the stomach wall between the first portion of the stomach wall and the second portion of the stomach wall, for creating the sensation of satiety.
6. The medical device according to aspect 3, wherein:
   the first member is elongated and comprises a first main length axis in the direction of the elongation, and
   the second member is elongated and comprises a second main length axis in the direction of the elongation, wherein
the operation device is configured to deform the first member, such that an angle between the first main length axis and the second main length axis is altered.
7. The medical device according to any one of the preceding aspects, wherein the operation device is connected to second member such that the connection with the second member provides counterforce against the force needed to move the at least a portion of the first member in a direction away from at least one portion of the second member.
8. The medical device according to any one of the preceding aspects, wherein the operation device is further configured to move at least a portion of the first member in a second direction away from at least one portion of the second member, for stretching a portion of the stomach wall between the first portion of the stomach wall and the second portion of the stomach wall for creating the sensation of satiety, wherein the second direction is different from the first direction.
9. The medical device according to any one of the preceding aspects, wherein the operation device comprises a first and a second operating member, and wherein the first operating member is configured to engage a first portion of the first member and the second operating member is configured to engage a second portion of the first member, such that:
   operation of the first operating member increases the distance between the first portion of the first member and a first portion of the second member, and
   operation of the second operating member increases the distance between the second portion of the first member and a second portion of the second member.
10. The medical device according to aspect 9, wherein: the first operating member is configured to displace the first portion of the first member relative to the first portion of the second member, and the second operating member is configured to displace the second portion of the first member relative to the second portion of the second member.
11. The medical device according to any one of aspects 9 and 10, wherein:
   the first operating member is configured to deform the first portion of the first member, and
   the second operating member is configured to deform the second portion of the first member.
12. The medical device according to any one of the preceding aspects, wherein the first member comprises at least one curvature.
13. The medical device according to aspect 12, wherein at least a portion of the first member forms a circular arc.
14. The medical device according to any one of aspects 12 and 13, wherein a portion of the first member is configured to deform, and wherein the portion of the first member configured to deform comprises at least one of the curvature and the circular arc.
15. The medical device according to any one of the preceding aspects, wherein the second member comprises at least one curvature.
16. The medical device according to aspect 15, wherein at least a portion of the second member forms a circular arc.
17. The medical device according to aspect 16, wherein at least a portion of the second member forms a circular arc.
18. The medical device according to aspect 17, wherein the circular are of the first member has a first radius, and the circular are of the second member has a second radius, and wherein the second radius is larger than the first radius.
19. The medical device according to any one of the preceding aspects, wherein at least one portion of the second member is fixedly fixated to the first member.
20. The medical device according to aspect 19, wherein the second member comprises a connecting portion for connecting the second member to the first member.
21. The medical device according to any one of the preceding aspects, wherein at least two portions of the second member is fixedly fixated to the first member.
22. The medical device according to any one of the preceding aspects, wherein the footprint of the first member is configured to enclose a portion of the stomach wall.
23. The medical device according to any one of the preceding aspects, wherein the footprint of the second member is configured to enclose a portion of the stomach wall.
24. The medical device according to any one of the preceding aspects, wherein the footprint of the first member is smaller than the footprint of the second member.
25. The medical device according to any one of the preceding aspects, wherein the second member is configured to encircle a portion of the stomach wall of the patient.
26. The medical device according to aspect 25, wherein the second member comprises a simple closed curve.
27. The medical device according to aspect 26, wherein the second member is ring shaped.
28. The medical device according to any one of the preceding aspects, wherein the first member is configured to encircle a portion of the stomach wall of the patient.
29. The medical device according to aspect 28, wherein the first member comprises a simple closed curve.
30. The medical device according to aspect 29, wherein the first member is ring shaped.
31. The medical device according to any one of the preceding aspects, wherein the first member comprises at least one flexible portion, and wherein the first member is configured to deform by deformation of the at least one flexible portion.
32. The medical device according to aspect 31, wherein the first flexible portion comprises at least one elastic portion.
33. The medical device according to aspect 32, wherein the elastic portion comprises at least one elastic material.
34. The medical device according to aspect 33, wherein the elastic material comprises an elastic polymer material.
35. The medical device according to aspect 34, wherein the elastic polymer material is an elastic polymer material selected from a list consisting of silicone-based materials and polyurethane-based materials.
36. The medical device according to any one of the preceding aspects, wherein the first member comprises at least one articulating joint, and wherein the first member is configured to deform by articulation of the at least one articulating joint.
37. The medical device according to any one of the preceding aspects, wherein the second member is configured to be at least partially invaginated by the stomach wall.
38. The medical device according to any one of the preceding aspects, wherein the first member is configured to be at least partially invaginated by the stomach wall.
39. The medical device according to any one of the preceding aspects, wherein the medical device is configured to be completely invaginated by the stomach wall, apart from elements for transferring force or communication to or from the medical device.
40. The medical device according to any one of the preceding aspects, wherein the first and second members are parts of a main portion placed in proximity to the stomach wall, and wherein a portion of the operation device is placed in the main portion, and wherein the portion of the operation device placed in the main portion comprises a receiving portion configured to receive mechanical force from the remote unit.
41. The medical device according to aspect 40, wherein the main portion comprises a transmission for transforming the received mechanical force into a force for operating the first member for stretching the stomach wall.
42. The medical device according to aspect 41, wherein the transmission comprises a gear system configured to reduce the speed and increase the force of the received mechanical force.
43. The medical device according to any one of the previous aspects, wherein the mechanical operation device comprises at least one flexible element, directly or indirectly fixated to the first member, wherein the flexible element is configured to operate the first member to move at least a portion of the first member in a first direction away from at least one portion of the second member.
44. The medical device according to aspect 43, wherein the main portion comprises at least one pulley, and wherein the at least one flexible element is configured to engage the pulley such that transmission is created.
45. The medical device according to aspect 44, wherein the transmission comprises:
a first and second pulley,
a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
46. The medical device according to aspect 44, wherein:
a first flexible element is configured to travel from a first point on the second member to the pulley such that the pulley is moved closer to the first point by the first flexible element being pulled in a direction towards the first point, and
the transmission created by the pulley causes the pulley to move towards the first point at a lower speed than the first flexible element moves towards the first point.
47. The medical device according to any one of aspects 44-46, wherein at least one pulley is connected to the first member.
48. The medical device according to any one of aspects 40-47, wherein the receiving portion is configured to receive a rotating mechanical force, and wherein the transmission is configured to transform the received rotating mechanical force into a liner mechanical force.
49. The medical device according to any one of aspects 40-47, wherein the receiving portion is configured to receive a linear mechanical force.
50. The medical device according to any one of the preceding aspects, wherein the main portion comprises at least one operating member in the form of a pulling member configured to pull at least a portion of the first member in a first direction away from at least one portion of the second member.
51. The medical device according to aspect 50, wherein the pulling member comprises the flexible element.
52. The medical device according to any one of aspects 43-51, wherein a first portion of the flexible element is directly or indirectly fixated to a first portion of the flexible element, and a second portion of the flexible element is directly or indirectly fixated to a second portion of the flexible element, and wherein the flexible element is configured to operate the first member such that the first and second portion is moved away from at least one portion of the second member.
53. The medical device according to aspect 43-52, wherein the flexible element is connected to a propulsion device for propelling the flexible element, and wherein:
propulsion of the flexible element in a first direction causes the first member to deform such that the first portion of the first member moves away from a first portion of the second member, and
   propulsion of the flexible element in a second direction causes the first member to deform such that the second portion of the first member moves away from a second portion of the second member.
54. The medical device according to any one of aspects 43-53, wherein the mechanical operation device comprises at least one second flexible element, directly or indirectly fixated to a second portion of the first member, and wherein the second flexible element is configured to operate the first member such that a second portion of the first member is moved away from a second portion of the second member, for stretching a portion of the stomach wall between the second portion of the first member and the second portion of the second member.
55. The medical device according to aspect 54, wherein the mechanical operation device comprises at least one third pulling member comprising at least one third flexible element, directly or indirectly fixated to a third portion of the first member, and wherein the third flexible element is configured to operate the first member such that a third portion of the first member is moved away from a third portion of the second member, for stretching a portion of the stomach wall between the third portion of the first member and the third portion of the second member.
56. The medical device according to any one of the preceding aspects, wherein the mechanical operation device comprises at least one movable arm having a first portion connected to the first member, and a second portion connected to the second member.
57. The medical device according to aspect 56, wherein the at least one movable arm comprises at least one operable joint.
58. The medical device according to any one of aspects 56-57, wherein the at least one movable arm is configured to deform the first member by moving the first portion of the first member away from the first portion of the second member.
59. The medical device according to any one of aspects 43-58, wherein the main portion comprises at least one recess or channel configured to house the flexible element.
60. The medical device according to any one of aspects 43-59, wherein the main portion comprises at least one sheath configured to house at least one of the flexible element and the pulley.
61. The medical device according to aspect 60, wherein the sheath comprises elevated and lowered portions, such that the sheath can remain flexible even as fibrotic tissue overgrows the sheath.
62. The medical device according to aspect 61, wherein at least a portion of the sheath is shaped like a bellows.
63. The medical device according to any one of aspects 60-62, wherein the sheath is connected to the first and second members, such that the flexible element can travel from the first to the second member through the sheath.
64. The medical device according to any one of the preceding aspects, wherein the second member is substantially rigid.
65. The medical device according to any one of the preceding aspects, wherein a major portion of the second member is made from a material having a modulus of elasticity in the range 50 GPa-400 GPa.
66. The medical device according to any one of the preceding aspects, wherein at least one of the length and the cross-sectional area of at least one of the first and second member is adjustable for calibrating the stretching of the stomach wall.
67. The medical device according to aspect 66, wherein at least one of the length and the cross-sectional area of at least one of the first and second member is mechanically adjustable, for calibrating the stretching of the stomach wall.
68. The medical device according to aspect 67, further comprising a rotatable threaded member for adjusting the length of at least one of the first and second member, for calibrating the stretching of the stomach wall.
69. The medical device according to aspect 67, wherein at least one of the length and the cross-sectional area of at least one of the first and second member is hydraulically adjustable, for calibrating the stretching of the stomach wall.
70. The medical device according to aspect 69, wherein at least one of the first and second member comprises at least one inflatable member, for calibrating the stretching of the stomach wall.
71. The medical device according to any one of aspects 66-70, wherein at least one of the first and second member comprises at least one portion comprising elevated and lowered areas for enabling adjustment of at least one of the length and the cross-sectional area of at least one of the first and second member.
72. The medical device according to aspect 71, wherein at least one of the first and second member comprises at least one bellows-shaped portion for enabling adjustment of at least one of the length and the cross-sectional area of at least one of the first and second member.
73. The medical device according to any one of the preceding aspects, wherein the portion of the operation device placed in the remote unit comprises at least one electrical motor. 74. The medical device according to aspect 73, wherein the portion of the operation device placed in the remote unit further comprises a gear system configured to reduce the speed and increase the force of the movement generated by the electrical motor.
75. The medical device according to any one of aspects 43-74, wherein the portion of the operation device placed in the remote unit comprises at least one pulley for propelling the flexible element.
76. The medical device according to any one of the preceding aspects, wherein the remote unit further comprises an energy storage unit for directly or indirectly energizing the medical device.
77. The medical device according to any one of the preceding aspects, wherein the remote unit further comprises a controller for controlling the operation device.
78. The medical device according to aspect 77, wherein the controller comprises a wireless transceiver for communicating wirelessly with a source external to the body of the patient.
79. The medical device according to any one of the preceding aspects, further comprising at least one sensor configured to sense a physical parameter of the medical device system.
80. The medical device according to aspect 79, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
81. The medical device according to any one of the preceding aspects, wherein the mechanical operation device is piezoelectric operation device.
82. The medical device according to aspect 81, wherein the piezoelectric operation device comprises a piezoelectric motor.
83. The medical device according to aspect 82, wherein the piezoelectric motor is a piezoelectric inchworm motor.
84. The medical device according to aspect 82, wherein the piezoelectric motor is a piezoelectric inertial motor.
85. The medical device according to aspect 82, wherein the piezoelectric motor is a piezoelectric walk-drive motor.
86. The medical device according to any one of aspects 81-85, wherein the piezoelectric operation device is a linear piezoelectric operation device.
87. The medical device according to aspect 86, wherein the linear piezoelectric operation device operates with at least one of:
   a speed in the range 1 mm/s to 10 mm/s,
   a stroke length in the range 4 mm-30 mm, and
   a force in the range 2N-30N.
88. The medical device according to any one of aspects 82-103, wherein the piezoelectric motor is a rotational piezoelectric motor.
89. The medical device according to aspect 88, wherein the rotational piezoelectric motor is configured to operate with at least one of:
   a rotational speed in the range 1 mrad/s-100 mrad/s, and
   a torque in the range 100 Nmm-900 Nmm.
90. The medical device according to aspect 82, wherein the piezoelectric motor is a piezoelectric ultrasonic motor.
91. The medical device according to aspect 90, wherein the piezoelectric ultrasonic motor is a traveling wave ultrasonic motor.
92. The medical device according to aspect 90, wherein the piezoelectric ultrasonic motor is a standing wave ultrasonic motor.
93. The medical device according to any one of aspects 90-92, wherein the piezoelectric ultrasonic motor is a rotational piezoelectric ultrasonic motor configured to operate with at least one of:
   a rotational speed in the range 10 mrad/s-10000 mrad/s, and
   a torque in the range 20 Nmm-450 Nmm.
94. The medical device according to any one of aspects 90-92, wherein the piezoelectric ultrasonic motor is a linear piezoelectric ultrasonic motor configured to operate with at least one of:
   a speed in the range 4 mm/s-10 mm/s, and
   a force in the range 0,5N-30N.
95. The medical device according to any one of aspects 81-94, wherein the piezoelectric operation device comprises at least one bimorph piezoelectric operation device.
96. The medical device according to any one of aspects 81-95, wherein the piezoelectric operation device is substantially non-magnetic.
97. The medical device according to any one of aspects 81-96, wherein the piezoelectric operation device is substantially non-metallic.
98. The medical device according to any one of aspects 81-97, wherein the piezoelectric operation device is a reversable piezoelectric operation device.

### Aspect 443—Obesity_Stretch_Version2_Several-Engaging-Points

1. A medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
2. The medical device according to aspect 1, wherein the operation device comprises:
   a propulsion device,
   a first operating element, and
   a second operating element, and wherein the mechanical switching device is configured to switch the operation device by altering a position of the propulsion device, such that the propulsion device in a first position operates the first operating element and in a second position operates the second operating element.
3. The medical device according to aspect 2, wherein at least one of the first and second operating element is configured to, directly or indirectly, pull at least one portion of the operable first member in a direction towards at least one portion of the second member.
4. The medical device according to aspect 3, further comprising an operable third member configured to be fixated to a third portion of the stomach wall, wherein the second operating element is configured to, directly or indirectly, pull at least one portion of the operable third member in a direction towards at least one portion of the second member.
5. The medical device according to aspect 3, further comprising at least one flexible element connected to the first operating element and, directly or indirectly, connected to the operable first member for pulling the operable first member in the first direction towards at least one portion of the second member.
6. The medical device according to aspect 4, further comprising at least one flexible element connected to the second operating element and, directly or indirectly, connected to at least one of the operable first member and operable third member, for pulling at least one of:
   the operable first member in the second direction towards at least one portion of the second member, and
   the operable third member in a direction towards at least one portion of the second member.
7. The medical device according to any one of aspects 4-6, wherein the at least one flexible element comprises a flexible wire.
8. The medical device according to any one of aspects 4-7, further comprising at least one pulley, and wherein the at least one flexible element is configured to engage the pulley such that transmission is created.
9. The medical device according to aspect 8, wherein the pulley is connected to one of the first member, the second member and the third member.
10. The medical device according to any one of aspects 4-9, wherein at least one of the first and second operating element comprises a drum or spool.
11. The medical device according to aspect 2, wherein at least one of the first and second operating element is configured to, directly or indirectly, push at least one portion of the operable first member in a direction away from at least one portion of the second member.
12. The medical device according to aspect 11, further comprising an operable third member configured to be fixated to a third portion of the stomach wall, wherein the second operating element is configured to, directly or indirectly, push at least one portion of the operable third member in a direction away from at least one portion of the second member.
13. The medical device according to any one of aspects 11 and 12, further comprising at least one shaft, rod or Bowden cable connected to the first operating element and, directly or indirectly, connected to the operable first member for pushing the operable first member in the first direction away from at least one portion of the second member.
14. The medical device according to aspect 13, further comprising at least one shaft, rod or Bowden cable connected to the second operating element and, directly or indirectly, connected to at least one of the operable first member and the operable third member, for pushing at least one of:
   the operable first member in the second direction away from at least one portion of the second member, and
   the operable third member in a direction away from at least one portion of the second member.
15. The medical device according to any one of aspects 4-14, further comprising at least one sheath configured to house at least one of the flexible element, the pulley, the shaft, the rod or the Bowden cable.
16. The medical device according to aspect 15, wherein the sheath comprises elevated and lowered portions, such that the sheath can remain flexible even as fibrotic tissue overgrows the sheath.
17. The medical device according to aspect 16, wherein at least a portion of the sheath is shaped like a bellows.
18. The medical device according to aspect 2, wherein at least one of the first and second operating element is configured to, directly or indirectly, pivot at least one portion of the operable first member relative to at least one portion of the second member.
19. The medical device according to aspect 18, further comprising an operable third member configured to be fixated to a third portion of the stomach wall, wherein the second operating element is configured to, directly or indirectly, pivot at least one portion of the operable third member relative to at least one portion of the second member.
20. The medical device according to any one of aspects 18 and 19, further comprising at least one screw, gear wheel or worm wheel connected to the first operating element and, directly or indirectly, connected to the operable first member for pivoting the operable first member relative to at least one portion of the second member.
21. The medical device according to aspect 20, further comprising at least one screw, gear wheel or worm wheel connected to the second operating element and, directly or indirectly, connected to at least one of the operable first member and operable third member, for pivoting at least one of:
   the operable first member relative to at least one portion of the second member, and
   the operable third member relative to at least one portion of the second member.
22. The medical device according to any one of aspects 2-21, wherein the propulsion device comprises a shaft and wherein the shaft comprises an engaging portion, and wherein the switch is configured to move the shaft in an axial direction, such that the engaging portion can move from a first position to a second position, and wherein the engaging portion:
   in the first position operatively engages the first operating element, and
   in the second position does not operatively engage the first operating element.
23. The medical device according to any one of aspects 2-21, wherein the propulsion device comprises a shaft and wherein the shaft comprises an engaging portion, and wherein the switch is configured to move the shaft in a substantially radial direction, such that the engaging portion can move from a first position to a second position, and wherein the engaging portion:
   in the first position operatively engages the first operating element, and
   in the second position does not operatively engage the first operating element.
24. The medical device according to any one of aspects 22 and 23, wherein the engaging portion:
   in the first position operatively engages the first operating element, and
   in the second position operatively engages the second operating element.
25. The medical device according to any one of aspects 22 and 23, wherein the shaft further comprises a second engaging portion, and wherein the switch is configured to move the shaft in an axial direction, such that the first and second engaging portions can move from a first position to a second position, and wherein:
   the first engaging portion, in the first position operatively engages the first operating element, and
   the second engaging portion, in the second position operatively engages the second operating element.
26. The medical device according to any one of aspects 22 and 23, wherein the shaft further comprises a second engaging portion, and wherein the switch is configured to move the shaft in a substantially radial direction, such that the first and second engaging portions can move from a first position to a second position, and wherein:
   the first engaging portion, in the first position operatively engages the first operating element, and
   the second engaging portion, in the second position operatively engages the second operating element.
27. The medical device according to aspect 25, wherein the operation device further comprises a third operating element and wherein the shaft further comprises a third engaging portion, and wherein the switch is configured to move the shaft in the axial direction of the shaft, such that the first, second and third engaging portions can move from the first position to the second position and from the second position to a third position, and wherein:
   the first engaging portion, in the first position operatively engages the first operating element,
   the second engaging portion, in the second position operatively engages the second operating element, and
   the third engaging portion, in the third position operatively engages the third operating element.
28. The medical device according to any one of aspects 22-27, wherein the shaft comprises at least one flexible portion.
29. The medical device according to any one of aspects 22-28, wherein the second member comprises at least one recess or channel configured to house the shaft.
30. The medical device according to any one of the preceding aspects, wherein the angle between the first and second directions exceeds 20°, preferably exceeds 30°, and most preferably exceeds 45°.
31. The medical device according to aspect 30, wherein the first direction is substantially opposite to the second direction.
32. The medical device according to any one of the preceding aspects, wherein the switch is an electrically powered switch.
33. The medical device according to aspect 32, wherein the switch comprises an electrical motor or solenoid.
34. The medical device according to any one of the preceding aspects, wherein the switch and the operable first member are operated by a single electrical motor.
35. The medical device according to any one of the preceding aspects, wherein the switch is operable by centrifugal force.
36. The medical device according to aspect 35, wherein the switch comprises a force output element operable by centrifugal force, such that the element:
   when rotated at a first speed engages a first force receiving element, and
   when rotated at a second speed engages a second force receiving element.
37. The medical device according to aspect 36, further comprising a shaft, and wherein:
   the first force receiving element is connected to the shaft such that rotation of the first force receiving element causes rotation in the shaft, and
   the second force receiving element comprises a transmission configured to transform a rotating force into a linear force, such that rotation of the first force receiving element causes linear movement of the rotation in the shaft.
38. The medical device according to aspect 37, wherein the linear movement causes the switch in the operation device from operating the first member to move in the first direction to operating the first member to move in the second direction, or from operating the first member to move in the first direction to operating the third member to move in the third direction.
39. The medical device according to any one of the preceding aspects, wherein the medical device comprises an electrical motor and a speed controller configured to control the speed of the electrical motor.
40. The medical device according to aspect 39, wherein the speed controller is configured to control the speed of the electrical motor, such that the electrical motor, at a first speed, engages the first force receiving element, at a second speed engages the second force receiving element.
41. The medical device according to any one of the preceding aspects, wherein the operation device is configured to be placed at least partially in a remote unit.
42. The medical device according to any one of the preceding aspects, wherein the switch is configured to be placed at least partially in a remote unit.
43. The medical device according to aspect 41, wherein operation device comprises an electrical motor placed in a remote unit.
44. The medical device according to any one of the preceding aspects, wherein operation device is configured to move the first or third member relative to the second member by displacing the first or third member relative to the second member.
45. The medical device according to aspect 44, wherein:
   the first member is elongated and comprises a first main length axis in the direction of the elongation, and
   the second member is elongated and comprises a second main length axis in the direction of the elongation, wherein the operation device is configured to displace the first member relative to the second member, such that an angle between the first main length axis and the second main length axis is altered.
46. The medical device according to any one of aspects 1-44, wherein the operation device is configured to move the first or third member relative to the second member by deforming the first or third member.
47. The medical device according to any one of the preceding aspects, wherein the operation device is connected to second member such that the connection with the second member provides counterforce against the force needed to move the at least a portion of the first or third member relative to the second member.
48. The medical device according to any one of the preceding aspects, wherein the first member comprises at least one curvature.
49. The medical device according to aspect 48, wherein at least a portion of the first member forms a circular arc.
50. The medical device according to any one of aspects 48 and 49, wherein a portion of the first member is configured to deform, and wherein the portion of the first member configured to deform comprises at least one of the curvature and the circular arc.
51. The medical device according to any one of the preceding aspects, wherein the second member comprises at least one curvature.
52. The medical device according to aspect 51, wherein at least a portion of the second member forms a circular arc.
53. The medical device according to aspect 52, wherein the circular are of the first member has a first radius, and the circular are of the second member has a second radius, and wherein the second radius is larger than the first radius.
54. The medical device according to any one of the preceding aspects, wherein at least one portion of the second member is fixedly fixated to the first member.
55. The medical device according to aspect 54, wherein the second member comprises a connecting portion for connecting the second member to the first member.
56. The medical device according to any one of the preceding aspects, wherein the footprint of the first member is configured to enclose a portion of the stomach wall.
57. The medical device according to any one of the preceding aspects, wherein the footprint of the second member is configured to enclose a portion of the stomach wall.
58. The medical device according to any one of the preceding aspects, wherein the footprint of the first member is smaller than the footprint of the second member.
59. The medical device according to any one of the preceding aspects, wherein the second member is configured to encircle a portion of the stomach wall of the patient.
60. The medical device according to aspect 59, wherein the second member comprises a simple closed curve.
61. The medical device according to aspect 60, wherein the second member is ring shaped.
62. The medical device according to any one of the preceding aspects, wherein the first member is configured to encircle a portion of the stomach wall of the patient.
63. The medical device according to aspect 62, wherein the first member comprises a simple closed curve.
64. The medical device according to aspect 63, wherein the first member is ring shaped.
65. The medical device according to any one of the preceding aspects, wherein the first member comprises at least one flexible portion, and wherein the first member is configured to deform by deformation of the at least one flexible portion.
66. The medical device according to aspect 65, wherein the first flexible portion comprises at least one elastic portion.
67. The medical device according to aspect 66, wherein the elastic portion comprises at least one elastic material.
68. The medical device according to any one of the preceding aspects, wherein the first member comprises at least one articulating joint, and wherein the first member is configured to deform by articulation of the at least one articulating joint.
69. The medical device according to any one of the preceding aspects, wherein the second member is configured to be at least partially invaginated by the stomach wall.
70. The medical device according to any one of the preceding aspects, wherein the first member is configured to be at least partially invaginated by the stomach wall.
71. The medical device according to any one of the preceding aspects, wherein the medical device is configured to be completely invaginated by the stomach wall, apart from elements for transferring force or communication to or from the medical device.
72. The medical device according to any one of the preceding aspects, wherein the first and second members are parts of a main portion placed in proximity to the stomach wall, and wherein a portion of the operation device is placed in the main portion, and wherein the portion of the operation device placed in the main portion comprises a receiving portion configured to receive mechanical force from the remote unit.
73. The medical device according to aspect 72, wherein the main portion comprises a transmission for transforming the received mechanical force into a force for operating the first member for stretching the stomach wall.
74. The medical device according to aspect 73, wherein the transmission comprises a gear or pulley system configured to reduce the speed and increase the force of the received mechanical force.
75. The medical device according to aspect 74, wherein:
   a first flexible element is configured to travel from a first point on the second member to a pulley such that the pulley is moved closer to the first point by the first flexible element being pulled in a direction towards the first point, and
   the transmission created by the pulley causes the pulley to move towards the first point at a lower speed than the first flexible element moves towards the first point.
76. The medical device according to any one of aspects 2-75, wherein at least one of the first operating element and the second operating element is configured to receive a rotating mechanical force, and wherein a transmission is configured to transform the received rotating mechanical force into a liner mechanical force.
77. The medical device according to any one of the preceding aspects, wherein the second member is substantially rigid.
78. The medical device according to any one of the preceding aspects, wherein a major portion of the second member is made from a material having a modulus of elasticity in the range 50 GPa-400 GPa.
79. The medical device according to any one of the preceding aspects, wherein at least one of the length and the cross-sectional area of at least one of the first and second member is adjustable for calibrating the stretching of the stomach wall.
80. The medical device according to any one of the preceding aspects, further comprising a remote unit configured to house at least a portion of the operation device.
81. The medical device according to aspect 80, wherein the remote unit further comprises at least one of:
   an energy storage unit for directly or indirectly energizing the medical device, and
   a controller for controlling the operation device.
82. The medical device according to aspect 81, wherein the controller comprises a wireless transceiver for communicating wirelessly with a source external to the body of the patient.
83. The medical device according to any one of the preceding aspects, further comprising at least one sensor configured to sense a physical parameter of the medical device system. 84. The medical device according to aspect 83, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect 435—Obesity_Stretch_Version2_Method

1. A method of implanting a medical device for actively stretching a stomach wall of a patient for creating a sensation of satiety, the medical device comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member,
for stretching a portion of the stomach wall of the patient between the second and first member, the method comprising:
making an incision in the abdomen of the patient, for accessing the stomach, dissecting a portion of the stomach,
inserting the medical device into the abdomen of the patient, fixating the second member to the stomach wall, and
fixating the first member to the stomach wall, within the footprint of the second member.
2. A method of implanting a medical device for actively stretching a stomach wall of a patient for creating a sensation of satiety, the medical device comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member, the method comprising:
making an incision in the abdomen of the patient, for accessing the stomach, dissecting a portion of the stomach,
inserting the medical device into the abdomen of the patient,
fixating the second member to the stomach wall, and
fixating the first member to the stomach wall, at least partially within the footprint of the second member.
3. A method of implanting a medical device for actively stretching a stomach wall of a patient for creating a sensation of satiety, the medical device comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety, the method comprising: making an incision in the abdomen of the patient, for accessing the stomach,
dissecting a portion of the stomach,
inserting the medical device into the abdomen of the patient,
fixating the operable first member to a first portion of the stomach wall, and
fixating the second member to a second portion of the stomach wall.
4. A method of implanting a medical device for actively stretching a stomach wall of a patient for creating a sensation of satiety, the medical device comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member, the method comprising: making an incision in the abdomen of the patient, for accessing the stomach, dissecting a portion of the stomach,
inserting the medical device into the abdomen of the patient,
fixating the operable first member to a first portion of the stomach wall, and
fixating the second member to a second portion of the stomach wall.
5. A method of implanting a medical device for actively stretching a stomach wall of a patient for creating a sensation of satiety, the medical device comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction, the method comprising: making an incision in the abdomen of the patient, for accessing the stomach, dissecting a portion of the stomach,
inserting the medical device into the abdomen of the patient,
fixating the operable first member to a first portion of the stomach wall, and
fixating the second member to a second portion of the stomach wall.
6. The method according to any one of aspect 1-5, wherein at least one of the steps of fixating the first member to a first portion of the stomach wall, and fixating the second member to a second portion of the stomach wall comprises at least partially invaginated at least one of the first and second members by the tissue of the stomach wall.
7. The method according to any one of aspect 1-6, wherein at least one of the steps of fixating the first member to a first portion of the stomach wall, and fixating the second member to a second portion of the stomach wall comprises fixating at least one of the first and second member to tissue of the stomach wall using at least one of sutures, staplers or tissue growth promoting structure.
8. The method according to any one of aspect 1-7, further comprising placing at least one sensor in the area of the esophagus of the patient for sensing a parameter related to the patient swallowing.
9. The method according to aspect 8, wherein the step of placing at least one sensor in the area of the esophagus of the patient for sensing a parameter related to the patient swallowing comprises placing at least one of: a motility sensor, a acoustic sensor, an optical sensor or a strain sensor.
10. The method according to any one of aspect 1-9, further comprising placing at least one sensor in the area of the stomach of the patient for sensing a parameter related to the patient eating.
11. The method according to aspect 10, wherein the step of placing at least one sensor in the area of the stomach of the patient for sensing a parameter related to the patient eating comprises placing at least one of temperature sensor, blood saturation or oxygenation sensor, a blood pressure sensor, a sensor configured to sense a parameter related to an ischemia marker, or a pH sensor.
12. The method according to aspect 11, wherein the step of sensing pH comprises sensing the acidity in the stomach.
13. The method according to any one of aspect 1-12, further comprising placing an electrode arrangement configured to be arranged between the medical device and the stomach to engage and electrically stimulate muscle tissue of the stomach to exercise the muscle tissue to improve the conditions for long term implantation of the medical device.
14. The method according to any one of aspect 1-13, further comprising placing an implantable remote unit connected to the medical device in the body of the patient.
15. The method according to aspect 14, wherein the step of placing an implantable remote unit connected to the medical device in the body of the patient comprises placing an implantable remote unit comprising at least a portion of the operation device.
16. The method according to any one of aspects 14 and 15, wherein the step of placing an implantable remote unit connected to the medical device in the body of the patient comprises placing an implantable remote unit comprising an energy storage unit.
17. The method according to any one of aspects 14-16, wherein the step of placing an implantable remote unit connected to the medical device in the body of the patient comprises placing an implantable remote unit comprising a wireless energy receiver.
18. The method according to any one of aspects 14-17, wherein the step of placing an implantable remote unit connected to the medical device in the body of the patient comprises placing an implantable remote unit comprising a wireless transceiver for wireless communication.

### Aspect 370—Obesity_Stretch_Version2_Z-Rod

1. A medical device for actively stretching a stomach wall of a patient for creating a sensation of satiety, the medical device being implantable and comprising:
   a first member, comprising:
      a first member rotating section, configured to rotate around a first axis,
      and a first member fixation section coupled to the first member rotating section and
      configured to be fixated to a first portion of the stomach wall; a second member coupled to the first member rotating section, the second member comprising a second member fixation section configured to be fixated to a second portion of the stomach wall; an operation device coupled to the first member rotating section, the operation device being configured to induce a rotation of the first member rotating section around the first axis; the first member fixation section being configured to describe a revolution around the first axis between a first position of the first member fixation section and a second position of the first member fixation section in response to the rotation of the first member rotating section;
wherein
   each of the first member rotating section and the operation device is positioned below a first reference plane perpendicular to the first axis, and the first member fixation section is positioned above said first reference plane for at least a part of the revolution of the first member fixation section;
   and the stomach wall between the first portion and second portion is increasingly stretched when the first member fixation section revolves towards the second position of the first member fixation section.
2. The medical device according to aspect 1, wherein the first member rotating section is configured to be at least partially invaginated by a tissue of the stomach wall.
3. The medical device according to any one of aspects 1-2, wherein at least one of the first member rotating section and first member fixation section is a rod with a circular cross-section area.
4. The medical device according to aspect 3, wherein the first member further comprises a first member coupling section connecting the first member rotating section and the first member fixation section.
5. The medical device according to aspect 4, wherein the first member coupling section has a circular cross-section area.
6. The medical device according to aspect 5, wherein the first member coupling section is positioned above the first reference plane.
7. The medical device according to aspect 6, wherein the first member coupling section is a rod arranged at angle in relation to the first axis.
8. The medical device according to any one of aspects 3-7, wherein the first member fixation section is parallel to the first member rotating section.
9. The medical device according to any one of aspects 1-8, wherein the medical device further comprises a main portion, and the operation device comprises a local portion placed in the main portion.
10. The medical device according to aspect 9, wherein the main portion is configured to be at least partially invaginated by a tissue of the stomach wall.
11. The medical device according to any one of aspects 1-10, wherein the first member fixation section is further configured to describe the revolution around the first axis maintaining a fixed distance from the first axis.
12. The medical device according to any one of aspects 9-10, wherein the local portion of the operation device comprises a first conversion device, the first conversion device comprising:
   a receiving portion configured to receive an input local force,
   and a transmission portion coupled to the receiving portion and to the first member rotating section, the transmission portion being configured to transform the input local force into an output local force inducing a rotation of the first member rotating section around the first axis.
13. The medical device according to aspect 12, wherein the receiving portion is arranged at a distance from the transmission portion, said distance being a characteristic distance of the first conversion device.
14. The medical device according to aspect 13, wherein the first conversion device is a first pulley, the first pulley having:
   a rim, configured to engage a flexible wire, where the rim is the receiving portion of the first conversion device,
   and a central portion, wherein the central portion is the transmission portion of the first conversion device.
15. The medical device according to aspect 14, wherein the rim of the first pulley is circular and the first pulley has a radius, the radius of the first pulley being the characteristic distance of the first pulley.
16. The medical device according to aspect 11 and any one of aspects 12-15, wherein the characteristic distance of the first conversion device is in a range of 0.3 times to 2 times as compared to the distance of the first member fixation section from the first axis.
17. The medical device according to aspect 11 and any one of aspects 12-15, wherein the characteristic distance of the first conversion device is in a range of 0.5 times to 1.2 times as compared to the distance of the first member fixation section from the first axis.
18. The medical device according to aspect 11 and any one of aspects 12-15, wherein the characteristic distance of the first conversion device is in a range of 0.95 times to 1.05 times as compared to the distance of the first member fixation section from the first axis.
19. The medical device according to any one of aspects 14-18, wherein the operation device comprises a first flexible wire configured to engage with the rim of the first pulley to induce a rotation of the first pulley.
20. The medical device according to aspect 19, wherein the first flexible wire configured to engage with the rim of the first pulley is further configured to pull the first pulley in a first rotating direction of the first pulley, and wherein the medical device further comprises a first spring mechanism coupled to the first pulley and configured to bias the first pulley towards a second rotating direction of the first pulley opposite to the first rotating direction of the first pulley.
21. The medical device according to aspect 19, wherein the first flexible wire configured to engage with the rim of the first pulley is further configured to pull the first pulley in a first rotating direction of the first pulley, and wherein the medical device further comprises a second flexible wire configured to engage with the rim of the first pulley to pull the first pulley in a second rotating direction of the first pulley opposite to the first rotating direction of the first pulley.
22. The medical device according to any one of aspects 12-21, wherein the local portion of the operation device further comprises a first local motor coupled to the receiving portion of the first conversion device, and the first local motor is configured to generate a movement having a first input local force and a first input local speed.
23. The medical device according to aspect 22, wherein the first local motor is a piezoelectric motor.
24. The medical device according to any one of aspects 22-23, wherein the first conversion device is a gear system configured to reduce the first input local speed and increase the first input local force.
25. The medical device according to any one of aspects 12-21, wherein the medical device further comprises a remote unit configured to be placed at a remote location in the body of the patient, and the operation device further comprises a remote portion placed in the remote unit.
26. The medical device according to aspect 25, wherein the remote portion of the operation device further comprises a first remote motor coupled to the receiving portion of the first conversion device and configured to generate a movement having a first input remote speed and a first input remote force.
27. The medical device according to aspect 26, wherein the first remote motor is a piezoelectric motor.
28. The medical device according to any one of aspects 26-27, wherein the remote portion of the operation device further comprises a first remote gear system configured to reduce the first input remote speed and increase the first input remote force.
29. The medical device according to any one of aspects 26-28, wherein the remote portion of the operation device further comprises a second remote motor coupled to the receiving portion of the first conversion device and configured to generate a movement having a second input remote speed and a second input remote force.
30. The medical device according to aspect 29, wherein the second remote motor is a piezoelectric motor.
31. The medical device according to any one of aspects 29-30, wherein the remote portion of the operation device further comprises a second remote gear system configured to reduce the second input remote speed and increase the second input remote force.
32. The medical device according to any one of aspects 26-31, wherein the operation device further comprises a first flexible wire configured to transmit the first input remote force from the first remote motor to the receiving portion of the first conversion device.
33. The medical device according to any one of aspects 29-31 and according to aspect 32, wherein the operation device further comprises a second flexible wire configured to transmit the second input remote force from the second remote motor to the receiving portion of the first conversion device, wherein the first flexible wire is configured to induce a rotation of the first member rotating section in a first rotating direction of the first member rotating section, and the second flexible wire is configured to induce a rotation of the first member rotating section in a second rotating direction of the first member rotating section opposite to the first rotating direction of the first member rotating section.
34. The medical device according to any one of aspects 26-31, wherein the operation device further comprises a first hydraulic system configured to transmit the first input remote force from the first remote motor to the receiving portion of the first conversion device.
35. The medical device according to aspect 34, wherein the first hydraulic system comprises:
a first hydraulic conduit;
a first remote hydraulic cylinder placed in the remote portion of the operation device, the first remote hydraulic cylinder being coupled to the first remote motor and to the first hydraulic conduit;
and a first local hydraulic cylinder placed in the local portion of the operation device, the first local hydraulic cylinder being coupled to the first hydraulic conduit and to the receiving portion of the first conversion device.
36. The medical device according to any one of aspects 29-31 and according to any one of aspects 34-35, wherein the operation device further comprises a second hydraulic system configured to transmit the second input remote force from the second remote motor to the receiving portion of the first conversion device, wherein the first hydraulic system is configured to induce a rotation of the first member rotating section in a first rotating direction of the first member rotating section, and the second hydraulic system is configured to induce a rotation of the first member rotating section in a second rotating direction of the first member rotating section opposite to the first rotating direction of the first member rotating section.
37. The medical device according to aspect 36, wherein the second hydraulic system comprises:
a second hydraulic conduit;
a second remote hydraulic cylinder placed in the remote portion of the operation device, the second remote hydraulic cylinder being coupled to the second remote motor and to the second hydraulic conduit;
and a second local hydraulic cylinder placed in the local portion of the operation device, the second local hydraulic cylinder being coupled to the second hydraulic conduit and to the receiving portion of the first conversion device.
38. The medical device according to aspect 9 and, optionally, any one of aspects 10-37, wherein the second member is inoperably fixated to the main portion.
39. The medical device according to aspect 38, wherein the second member is configured to be at least partially invaginated by a tissue of the stomach wall.
40. The medical device according to aspect 39, wherein the second member is a rod having a circular cross-section area.
41. The medical device according to aspect 40, wherein the second member is parallel to the first member fixation section.
42. The medical device according to any one of aspects 1-41, wherein the medical device further comprises:
   a third member coupled to the first member rotating section, the third member comprising a third
   member fixation section configured to be fixated to a third portion of the stomach wall; and wherein:
   the stomach wall between the first portion and third portion is increasingly stretched when the first member fixation section revolves towards the first position of the first member fixation section.
43. The medical device according to any one of aspects 38-41 and aspect 42, wherein the third member is inoperably fixated to the main portion.
44. The medical device according to aspect 43, wherein the third member is configured to be at least partially invaginated by a tissue of the stomach wall.
45. The medical device according to aspect 44, wherein the third member is a rod having a circular cross-section area.
46. The medical device according to aspect 45, wherein the third member is parallel to the first member fixation section.
47. The medical device according to any one of aspects 1-37, wherein the second member further comprises:
   a second member rotating section coupled to the first member rotating section and configured to rotate around a second axis,
   and a second member fixation section coupled to the second member rotating section and configured to be fixated to the second portion of the stomach wall; and wherein:
   the operation device is further coupled to the second member rotating section and further configured to induce a rotation of the second member rotating section around the second axis; the second member fixation section is configured to describe a revolution around the second axis between a first position of the second member fixation section and a second position of the second member fixation section in response to the rotation of the second member rotating section;
   each of the second member rotating section and the operation device is positioned below a second reference plane perpendicular to the second axis, and the second member fixation section is positioned above said second reference plane for at least a part of the revolution of the second member fixation section;
   and the stomach wall between the first portion and second portion is increasingly stretched when the second member fixation section revolves towards the first position of the second member fixation section.
48. The medical device according to aspect 47, wherein the second member rotating section is configured to be at least partially invaginated by a tissue of the stomach wall.
49. The medical device according to any one of aspects 47-48, wherein at least one of the second member rotating section and second member fixation section is a rod with a circular cross-section area.
50. The medical device according to aspect 49, wherein the second member further comprises a second member coupling section connecting the second member rotating section and the second member fixation section.
51. The medical device according to aspect 50, wherein the second member coupling section has a circular cross-section area.
52. The medical device according to aspect 51, wherein the second member coupling section is positioned above the second reference plane.
53. The medical device according to aspect 52, wherein the second member coupling section is a rod arranged at angle in relation to the second axis.
54. The medical device according to any one of aspects 49-53, wherein the second member fixation section is parallel to the second member rotating section.
55. The medical device according to any one of aspects 49-54, wherein the second member fixation section is parallel to the first member fixation section.
56. The medical device according to any one of aspects 47-55, wherein the second axis is parallel to the first axis.
57. The medical device according to any one of aspects 47-56, wherein the second member fixation section is further configured to describe the revolution around the second axis maintaining a fixed distance from the second axis.
58. The medical device according to any one of aspects 9-10 and any one of aspects 47-57, wherein the local portion of the operation device comprises a second conversion device, the second conversion device comprising:
   a receiving portion configured to receive an input local force, and a transmission portion coupled to the receiving portion of the second conversion device and to the second member rotating section, the transmission portion of the second conversion device being configured to transform the input local force received by the receiving portion of the second conversion device into an output local force inducing a rotation of the second member rotating section around the second axis.
59. The medical device according to aspect 58, wherein the receiving portion of the second conversion device is arranged at a distance from the transmission portion of the second conversion device, said distance being a characteristic distance of the second conversion device.
60. The medical device according to aspect 59, wherein the second conversion device is a second pulley, the second pulley having:
   a rim, configured to engage a flexible wire, where the rim of the second pulley is the receiving portion of the second conversion device,
   and a central portion, wherein the central portion of the second pulley is the transmission portion of the second conversion device.
61. The medical device according to aspect 60, wherein the rim of the second pulley is circular and the second pulley has a radius, the radius of the second pulley being the characteristic distance of the second pulley.
62. The medical device according to aspect 57 and any one of aspects 58-61, wherein the characteristic distance of the second conversion device is in a range of 0.3 times to 2 times as compared to the distance of the second member fixation section from the second axis.
63. The medical device according to aspect 57 and any one of aspects 58-61, wherein the characteristic distance of the second conversion device is in a range of 0.5 times to 1.2 times as compared to the distance of the second member fixation section from the second axis.
64. The medical device according to aspect 57 and any one of aspects 58-61, wherein the characteristic distance of the second conversion device is in a range of 0.95 times to 1.05 times as compared to the distance of the second member fixation section from the second axis.
65. The medical device according to any one of aspects 60-64, wherein the operation device comprises a third flexible wire configured to engage with the rim of the second pulley to induce a rotation of the second pulley.
66. The medical device according to aspect 65, wherein the third flexible wire configured to engage with the rim of the second pulley is further configured to pull the second pulley in a first rotating direction of the second pulley, and wherein the medical device further comprises a second spring mechanism coupled to the second pulley and configured to bias the second pulley towards a second rotating direction of the second pulley opposite to the first rotating direction of the second pulley.
67. The medical device according to aspect 65, wherein the third flexible wire configured to engage with the rim of the second pulley is further configured to pull the second pulley in a first rotating direction of the second pulley, and wherein the medical device further comprises a fourth flexible wire configured to engage with the rim of the second pulley to pull the second pulley in a second rotating direction of the second pulley opposite to the first rotating direction of the second pulley.
68. The medical device according to any one of aspects 19-20 and any one of aspects 65-66, wherein the first flexible wire configured to engage with the rim of the first pulley is the same as the third flexible wire configured to engage with the rim of the second pulley, and is configured to pull the first pulley and the second pulley in opposite rotating directions.
69. The medical device according to aspect 21 and aspect 67, wherein the first flexible wire configured to engage with the rim of the first pulley is the same as the third flexible wire configured to engage with the rim of the second pulley, the second flexible wire configured to engage with the rim of the first pulley is the same as the fourth flexible wire configured to engage with the rim of the second pulley, the first rotating direction of the first pulley is opposite to the first rotating direction of the second pulley, and the second rotating direction of the first pulley is opposite to the second rotating direction of the second pulley.
70. The medical device according to any one of aspects 58-69, wherein the local portion of the operation device further comprises a second local motor coupled to the receiving portion of the second conversion device, and the second local motor is configured to generate a movement having a second input local force and a second input local speed.
71. The medical device according to aspect 70, wherein the second local motor is a piezoelectric motor.
72. The medical device according to any one of aspects 70-71, wherein the second conversion device is a gear system configured to reduce the second input local speed and increase the second input local force.
73. The medical device according to aspect 26-31 and any one of aspects 58-69, wherein the remote portion of the operation device further comprises a third remote motor coupled to the receiving portion of the second conversion device and configured to generate a movement having a third input remote speed and a third input remote force.
74. The medical device according to aspect 73, wherein the third remote motor is a piezoelectric motor.
75. The medical device according to any one of aspects 73-74, wherein the remote portion of the operation device further comprises a third remote gear system configured to reduce the third input remote speed and increase the third input remote force.
76. The medical device according to any one of aspects 73-75, wherein the remote portion of the operation device further comprises a fourth remote motor coupled to the receiving portion of the second conversion device and configured to generate a movement having a fourth input remote speed and a fourth input remote force.
77. The medical device according to aspect 76, wherein the fourth remote motor is a piezoelectric motor.
78. The medical device according to any one of aspects 76-77, wherein the remote portion of the operation device further comprises a fourth remote gear system configured to reduce the fourth input remote speed and increase the fourth input remote force.
79. The medical device according to any one of aspects 73-78, wherein the operation device further comprises a third flexible wire configured to transmit the third input remote force from the third remote motor to the receiving portion of the second conversion device.
80. The medical device according to any one of aspects 76-78 and according to aspect 79, wherein the operation device further comprises a fourth flexible wire configured to transmit the fourth input remote force from the fourth remote motor to the receiving portion of the second conversion device, wherein the third flexible wire is configured to induce a rotation of the second member rotating section in a first rotating direction of the second member rotating section, and the fourth flexible wire is configured to induce a rotation of the second member rotating section in a second rotating direction of the second member rotating section opposite to the first rotating direction of the second member rotating section.
81. The medical device according to aspect 32 and aspect 79, wherein the first flexible wire is the same as the third flexible wire, the first remote motor is the same as the third remote motor, and the first flexible wire is configured to rotate the first member rotating section and second member rotating section in opposite rotating directions.
82. The medical device according to aspect 33 and aspect 80, wherein the first flexible wire is the same as the third flexible wire, the first remote motor is the same as the third remote motor, the second flexible wire is the same as the fourth flexible wire and the second remote motor is the same as the fourth remote motor, and wherein the first rotating direction of the first member rotating section is opposite to the first rotating direction of the second member rotating section, and the second rotating direction of the first member rotating section is opposite to the second rotating direction of the second member rotating section.
83. The medical device according to any one of aspects 73-78, wherein the operation device further comprises a third hydraulic system configured to transmit the third input remote force from the third remote motor to the receiving portion of the second conversion device.
84. The medical device according to aspect 83, wherein the third hydraulic system comprises:
   a third hydraulic conduit;
   a third remote hydraulic cylinder placed in the remote portion of the operation device, the third remote hydraulic cylinder being coupled to the third remote motor and to the third hydraulic conduit;
   and a third local hydraulic cylinder placed in the local portion of the operation device, the third local hydraulic cylinder being coupled to the third hydraulic conduit and to the receiving portion of the second conversion device.
85. The medical device according to any one of aspects 76-78 and according to any one of aspects 83-84, wherein the operation device further comprises a fourth hydraulic system configured to transmit the fourth input remote force from the fourth remote motor to the receiving portion of the second conversion device, wherein the third hydraulic system is configured to induce a rotation of the second member rotating section in a first rotating direction of the second member rotating section, and the fourth hydraulic system is configured to induce a rotation of the second member rotating section in a second rotating direction of the second member rotating section opposite to the first rotating direction of the second member rotating section.
86. The medical device according to aspect 85, wherein the fourth hydraulic system comprises:
   a fourth hydraulic conduit;
   a fourth remote hydraulic cylinder placed in the remote portion of the operation device, the fourth remote hydraulic cylinder being coupled to the fourth remote motor and to the fourth hydraulic conduit;
   and a fourth local hydraulic cylinder placed in the local portion of the operation device, the fourth local hydraulic cylinder being coupled to the fourth hydraulic conduit and to the receiving portion of the second conversion device.
87. The medical device according to any one of aspects 34-35 and any one of aspects 83-84, wherein the first hydraulic system is the same as the third hydraulic system, the first remote motor is the same as the third remote motor, and the first hydraulic system is configured to rotate the first member rotating section and second member rotating section in opposite rotating directions.
88. The medical device according to any one of aspects 36-37 and any one of aspects 85-86, wherein the first hydraulic system is the same as the third hydraulic system, the first remote motor is the same as the third remote motor, the second hydraulic system is the same as the fourth hydraulic system and the second remote motor is the same as the fourth remote motor, and wherein the first rotating direction of the first member rotating section is opposite to the first rotating direction of the second member rotating section, and the second rotating direction of the first member rotating section is opposite to the second rotating direction of the second member rotating section.
89. The medical device according to any one of aspects 1-41, wherein the medical device further comprises:
   a third member comprising:
      a third member rotating section coupled to the second member and configured to rotate around a third axis,
      and a third member fixation section coupled to the third member rotating section and configured to be fixated to a third portion of the stomach wall;
and wherein:
   the operation device is further coupled to the third member rotating section and further configured to induce a rotation of the third member rotating section around the third axis; the third member fixation section is configured to describe a revolution around the third axis between a first position of the third member fixation section and a second position of the third member fixation section in response to the rotation of the third member rotating section; each of the third member rotating section and the operation device is positioned below a third reference plane perpendicular to the third axis, and the third member fixation section is positioned above said third reference plane for at least a part of the revolution of the third member fixation section;
   and the stomach wall between the second portion and third portion is increasingly stretched when the third member fixation section revolves towards the first position of the third member.
90. The medical device according to aspect 89, wherein the third member rotating section is configured to be at least partially invaginated by a tissue of the stomach wall.
91. The medical device according to any one of aspects 89-90, wherein at least one of the third member rotating section and third member fixation section is a rod with a circular cross-section area.
92. The medical device according to aspect 91, wherein the third member further comprises a third member coupling section connecting the third member rotating section and the third member fixation section.
93. The medical device according to aspect 92, wherein the third member coupling section has a circular cross-section area.
94. The medical device according to aspect 93, wherein the third member coupling section is positioned above the third reference plane.
95. The medical device according to aspect 94, wherein the third member coupling section is a rod arranged at angle in relation to the third axis.
96. The medical device according to any one of aspects 91-95, wherein the third member fixation section is parallel to the third member rotating section.
97. The medical device according to any one of aspects 89-96, wherein the third axis is parallel to the first axis.
98. The medical device according to any one of aspects 89-97, wherein the third member fixation section is further configured to describe the revolution around the third axis maintaining a fixed distance from the third axis.
99. The medical device according to any one of aspects 9-10 and any one of aspects 89-98, wherein the local portion of the operation device comprises a second conversion device, the second conversion device comprising:
   a receiving portion configured to receive an input local force, and a transmission portion coupled to the receiving portion of the second conversion device and to the third member rotating section, the transmission portion of the second conversion device being configured to transform the input local force received by the receiving portion of the second conversion device into an output local force inducing a rotation of the third member rotating section around the third axis.
100. [PTO2][PTO3] The medical device according to aspect 99, wherein the receiving portion of the second conversion device is arranged at a distance from the transmission portion of the second conversion device, said distance being a characteristic distance of the second conversion device.
101. [PTO4] The medical device according to aspect 100, wherein the second conversion device is a second pulley, the second pulley having:
   a rim, configured to engage a flexible wire, where the rim of the second pulley is the receiving portion of the second conversion device,
   and a central portion, wherein the central portion of the second pulley is the transmission portion of the second conversion device.
102. [PTO5] The medical device according to aspect 101, wherein the rim of the second pulley is circular and the second pulley has a radius, the radius of the second pulley being the characteristic distance of the second pulley.
103. [PTO6] The medical device according to aspect 98 and any one of aspects 99-102, wherein the characteristic distance of the second conversion device is in a range of 0.3 times to 2 times as compared to the distance of the third member fixation section from the third axis.
104. [PTO7] The medical device according to aspect 98 and any one of aspects 99-102, wherein the characteristic distance of the second conversion device is in a range of 0.5 times to 1.2 times as compared to the distance of the third member fixation section from the third axis.
105. [PTO8] The medical device according to aspect 98 and any one of aspects 99-102, wherein the characteristic distance of the second conversion device is in a range of 0.95 times to 1.05 times as compared to the distance of the third member fixation section from the third axis.
106. [PTO9] The medical device according to any one of aspects 101-105, wherein the operation device comprises a third flexible wire configured to engage with the rim of the second pulley to induce a rotation of the second pulley.
107. [PTO10] The medical device according to aspect 106, wherein the third flexible wire configured to engage with the rim of the second pulley is further configured to pull the second pulley in a first rotating direction of the second pulley, and wherein the medical device further comprises a second spring mechanism coupled to the second pulley and configured to bias the second pulley towards a second rotating direction of the second pulley opposite to the first rotating direction of the second pulley.
108. [PTO11] The medical device according to aspect 106, wherein the third flexible wire configured to engage with the rim of the second pulley is further configured to pull the second pulley in a first rotating direction of the second pulley, and wherein the medical device further comprises a fourth flexible wire configured to engage with the rim of the second pulley to pull the second pulley in a second rotating direction of the second pulley opposite to the first rotating direction of the second pulley.
109. [PTO12] The medical device according to any one of aspects 19-20 and any one of aspects 106-107, wherein the first flexible wire configured to engage with the rim of the first pulley is the same as the third flexible wire configured to engage with the rim of the second pulley, and is configured to pull the first pulley and the second pulley in opposite rotating directions.
110. The medical device according to aspect 21 and aspect 108, wherein the first flexible wire configured to engage with the rim of the first pulley is the same as the third flexible wire configured to engage with the rim of the second pulley, the second flexible wire configured to engage with the rim of the first pulley is the same as the fourth flexible wire configured to engage with the rim of the second pulley, the first rotating direction of the first pulley is opposite to the first rotating direction of the second pulley, and the second rotating direction of the first pulley is opposite to the second rotating direction of the second pulley.
111. The medical device according to any one of aspects 99-110, wherein the local portion of the operation device further comprises a second local motor coupled to the receiving portion of the second conversion device, and the second local motor is configured to generate a movement having a second input local force and a second input local speed.
112. The medical device according to aspect 111, wherein the second local motor is a piezoelectric motor.
113. The medical device according to any one of aspects 111-112, wherein the second conversion device is a gear system configured to reduce the second input local speed and increase the second input local force.
114. The medical device according to aspect 26-31 and any one of aspects 99-110, wherein the remote portion of the operation device further comprises a third remote motor coupled to the receiving portion of the second conversion device and configured to generate a movement having a third input remote speed and a third input remote force.
115. The medical device according to aspect 114, wherein the third remote motor is a piezoelectric motor.
116. The medical device according to any one of aspects 114-115, wherein the remote portion of the operation device further comprises a third remote gear system configured to reduce the third input remote speed and increase the third input remote force.
117. The medical device according to any one of aspects 114-116, wherein the remote portion of the operation device further comprises a fourth remote motor coupled to the receiving portion of the second conversion device and configured to generate a movement having a fourth input remote speed and a fourth input remote force.
118. The medical device according to aspect 117, wherein the fourth remote motor is a piezoelectric motor.
119. The medical device according to any one of aspects 117-118, wherein the remote portion of the operation device further comprises a fourth remote gear system configured to reduce the fourth input remote speed and increase the fourth input remote force.
120. The medical device according to any one of aspects 114-119, wherein the operation device further comprises a third flexible wire configured to transmit the third input remote force from the third remote motor to the receiving portion of the second conversion device.
121. The medical device according to any one of aspects 117-119 and according to aspect 120, wherein the operation device further comprises a fourth flexible wire configured to transmit the fourth input remote force from the fourth remote motor to the receiving portion of the second conversion device, wherein the third flexible wire is configured to induce a rotation of the third member rotating section in a first rotating direction of the third member rotating section, and the fourth flexible wire is configured to induce a rotation of the third member rotating section in a second rotating direction of the third member rotating section opposite to the first rotating direction of the third member rotating section.
122. The medical device according to aspect 32 and aspect 120, wherein the first flexible wire is the same as the third flexible wire, the first remote motor is the same as the third remote motor, and the first flexible wire is configured to rotate the first member rotating section and third member rotating section in opposite rotating directions.
123. The medical device according to aspect 33 and aspect 121, wherein the first flexible wire is the same as the third flexible wire, the first remote motor is the same as the third remote motor, the second flexible wire is the same as the fourth flexible wire and the second remote motor is the same as the fourth remote motor, and wherein the first rotating direction of the first member rotating section is opposite to the first rotating direction of the third member rotating section, and the second rotating direction of the first member rotating section is opposite to the second rotating direction of the third member rotating section.
124. The medical device according to any one of aspects 114-119, wherein the operation device further comprises a third hydraulic system configured to transmit the third input remote force from the third remote motor to the receiving portion of the second conversion device.
125. The medical device according to aspect 124, wherein the third hydraulic system comprises:
   a third hydraulic conduit;
   a third remote hydraulic cylinder placed in the remote portion of the operation device, the third remote hydraulic cylinder being coupled to the third remote motor and to the third hydraulic conduit;
   and a third local hydraulic cylinder placed in the local portion of the operation device, the third local hydraulic cylinder being coupled to the third hydraulic conduit and to the receiving portion of the second conversion device.
126. The medical device according to any one of aspects 117-119 and according to any one of aspects 124-125, wherein the operation device further comprises a fourth hydraulic system configured to transmit the fourth input remote force from the fourth remote motor to the receiving portion of the second conversion device, wherein the third hydraulic system is configured to induce a rotation of the third member rotating section in a first rotating direction of the third member rotating section, and the fourth hydraulic system is configured to induce a rotation of the third member rotating section in a second rotating direction of the third member rotating section opposite to the first rotating direction of the third member rotating section.
127. The medical device according to aspect 126, wherein the fourth hydraulic system comprises:
   a fourth hydraulic conduit;
   a fourth remote hydraulic cylinder placed in the remote portion of the operation device, the fourth remote hydraulic cylinder being coupled to the fourth remote motor and to the fourth hydraulic conduit;
   and a fourth local hydraulic cylinder placed in the local portion of the operation device, the fourth local hydraulic cylinder being coupled to the fourth hydraulic conduit and to the receiving portion of the second conversion device.
128. The medical device according to any one of aspects 34-35 and any one of aspects 124-125, wherein the first hydraulic system is the same as the third hydraulic system, the first remote motor is the same as the third remote motor, and the first hydraulic system is configured to rotate the first member rotating section and third member rotating section in opposite rotating directions.
129. The medical device according to any one of aspects 36-37 and any one of aspects 126-127, wherein the first hydraulic system is the same as the third hydraulic system, the first remote motor is the same as the third remote motor, the second hydraulic system is the same as the fourth hydraulic system and the second remote motor is the same as the fourth remote motor, and wherein the first rotating direction of the first member rotating section is opposite to the first rotating direction of the third member rotating section, and the second rotating direction of the first member rotating section is opposite to the second rotating direction of the third member rotating section.

### Aspect 511—Obesity_Stretch_Version2_Piezo

1. A medical device for actively stretching wall portion of a stomach or intestine of a human for creating a sensation of satiety, the medical device being implantable and comprising:
   an operable first member configured to be fixated to a first portion of the wall of the stomach or intestine,
   an operation device configured to at least one of:
      move at least a portion of the first member in a first direction, and
      alter the first member,
such that the wall of the stomach or intestine is actively stretched for creating a sensation of satiety.
2. The medical device according to aspect 1, wherein the operable first member comprises at least one curved portion configured to be fixated to the first portion of the wall of the stomach or intestine, and wherein the operation device is configured alter the curvature of the first member for actively stretching the first portion of the intestinal wall.
3. The medical device according to any one of aspects 1-2, further comprising a second member configured to be fixated to a second portion of the wall of the stomach or intestine, and wherein the operation device is configured to move at least a portion of the first member in a first direction away from the second member, for stretching the intestinal wall between the first and second portion of the stomach or intestine.
4. The medical device according to aspect 3, wherein the operation device is configured to move at least a portion of the first member in a first direction away from the second member, for stretching the wall of the stomach or intestine between the first and second portion of the stomach or intestine.
5. The medical device according to any one of the preceding aspects, wherein the operation device is configured to alter the operable first member by increasing the volume of the first member.
6. The medical device according to any one of the preceding aspects, wherein at least one of the first and second member is configured to be invaginated in the intestinal wall.
7. The medical device according to any one of the preceding aspects, wherein the first member is configured to be completely invaginated by the intestinal wall, apart from elements for transferring force or communication to or from the first member.
8. The medical device according to any one of the preceding aspects, wherein the first member is mechanically operable, and the operation device is a mechanical operation device.
9. The medical device according to aspect 8, wherein the mechanical operation device comprises a pulling member for operating the first member.
10. The medical device according to aspect 9, wherein the pulling member comprises at least one flexible element, directly or indirectly fixated to the first member, wherein the flexible element is configured to operate the first member by pulling at least a portion of the first member.
11. The medical device according to any one of aspects 1-10, wherein the first member is hydraulically operable, and the operation device is a hydraulic operation device.
12. The medical device according to aspect 11, wherein the pulling member comprises at least one hydraulic actuator, directly or indirectly fixated to at least one of the first and second members.
13. The medical device according to any one of the preceding aspects, wherein at least a portion of the operation device is placed in a remote unit.
14. The medical device according to any one of the preceding aspects, wherein the operation device is configured to pull at least a portion of the first member in a first direction away from at least one portion of the second member, for stretching a portion of the wall of the stomach or intestine between the first portion of the wall of the stomach or intestine and the second portion of the wall of the stomach or intestine for creating the sensation of satiety.
15. The medical device according to any one of the preceding aspects, wherein the first member is configured to be operated to deform such that a first portion of the first member is moved away from a first portion of the second member, for stretching a portion of the wall of the stomach or intestine between the first portion of the first member and the first portion of the second member.
16. The medical device according to any one of aspects 1-15, wherein the operation device is a piezoelectric operation device.
17. The medical device according to aspect 16, wherein the piezoelectric operation device comprises a piezoelectric motor.
18. The medical device according to aspect 17, wherein the piezoelectric motor is a piezoelectric inchworm motor.
19. The medical device according to aspect 17, wherein the piezoelectric motor is a piezoelectric inertial motor.
20. The medical device according to aspect 17, wherein the piezoelectric motor is a piezoelectric walk-drive motor.
21. The medical device according to any one of aspects 16-20, wherein the piezoelectric operation device is a linear piezoelectric operation device.
22. The medical device according to aspect 21, wherein the linear piezoelectric operation device operates with at least one of:
   a speed in the range 1 mm/s to 10 mm/s,
   a stroke length in the range 4 mm-30 mm, and
   a force in the range 2N-30N.
23. The medical device according to any one of aspects 17-22, wherein the piezoelectric motor is a rotational piezoelectric motor.
24. The medical device according to aspect 23, wherein the rotational piezoelectric motor is configured to operate with at least one of:
   a rotational speed in the range 1 mrad/s-100 mrad/s, and
   a torque in the range 100 Nmm-900 Nmm.
25. The medical device according to aspect 17, wherein the piezoelectric motor is a piezoelectric ultrasonic motor.
26. The medical device according to aspect 25, wherein the piezoelectric ultrasonic motor is a traveling wave ultrasonic motor.
27. The medical device according to aspect 25, wherein the piezoelectric ultrasonic motor is a standing wave ultrasonic motor.
28. The medical device according to any one of aspects 25-27, wherein the piezoelectric ultrasonic motor is a rotational piezoelectric ultrasonic motor configured to operate with at least one of: a rotational speed in the range 10 mrad/s-10000 mrad/s, and a torque in the range 20 Nmm-450 Nmm.
29. The medical device according to any one of aspects 25-27, wherein the piezoelectric ultrasonic motor is a linear piezoelectric ultrasonic motor configured to operate with at least one of:
   a speed in the range 4 mm/s-10 mm/s, and
   a force in the range 0,5N-30N.
30. The medical device according to any one of aspects 16-29, wherein the piezoelectric operation device comprises at least one bimorph piezoelectric operation device.
31. The medical device according to any one of aspects 16-30, wherein the piezoelectric operation device is substantially non-magnetic.
32. The medical device according to any one of aspects 16-31, wherein the piezoelectric operation device is substantially non-metallic.
33. The medical device according to any one of aspects 16-32, wherein the piezoelectric operation device is a reversable piezoelectric operation device.

### Aspect 512—Obesity_Stretch_Version2_Intestine

1. A medical device for actively stretching an intestinal wall of a human for creating a sensation of satiety, the medical device being implantable and comprising:
   an operable first member configured to be fixated to a first portion of the intestinal wall,
   an operation device configured to at least one of:
      move at least a portion of the first member in a first direction, and
      alter the first member,
such that the intestinal wall is actively stretched for creating a sensation of satiety.
2. The medical device according to aspect 1, wherein the operable first member comprises at least one curved portion configured to be fixated to the first portion of the intestinal wall, and wherein the operation device is configured alter the curvature of the first member for actively stretching the first portion of the intestinal wall.
3. The medical device according to aspect 2, wherein the curved portion of the operable first member is configured to at least partially enclose the first portion of the intestinal wall.
4. The medical device according to aspect 3, wherein the curved portion of the operable first member forms a closed curve configured to enclose the intestinal wall.
5. The medical device according to aspect 4, wherein the closed curve comprises a circular closed curve configured to enclose the intestinal wall.
6. The medical device according to any one of aspects 2-5, wherein the operable first member is configured to be fixated to the first portion of the intestinal wall at a plurality of positions, and wherein the operation device is configured to alter the operable first member by increasing a length of the first member for actively stretching the intestinal wall.
7. The medical device according to aspect 6, wherein the operation device is configured to alter the operable first member by increasing the length of the closed curve.
8. The medical device according to aspect 7, wherein the operation device is configured to alter the operable first member by increasing the circumference of the circular closed curve.
9. The medical device according to any one of aspects 1-8, further comprising a second member configured to be fixated to a second portion of the intestinal wall, and wherein the operation device is configured to move at least a portion of the first member in a first direction away from the second member, for stretching the intestinal wall between the first and second portion.
10. The medical device according to aspect 9, wherein the operation device is configured to move at least a portion of the first member in a first direction away from the second member, for stretching the intestinal wall between the first and second portion substantially in an axial direction of the intestine.
11. The medical device according to any one of the preceding aspects, wherein the operation device is configured to alter the operable first member by increasing the volume of the first member.
12. The medical device according to any one of the preceding aspects, wherein at least one of the first and second member is configured to be invaginated in the intestinal wall.
13. The medical device according to any one of the preceding aspects, wherein the first member is configured to be completely invaginated by the intestinal wall, apart from elements for transferring force or communication to or from the first member.
14. The medical device according to any one of the preceding aspects, wherein the first member is mechanically operable, and the operation device is a mechanical operation device.
15. The medical device according to aspect 14, wherein the mechanical operation device comprises a pulling member for operating the first member.
16. The medical device according to aspect 15, wherein the pulling member comprises at least one flexible element, directly or indirectly fixated to the first member, wherein the flexible element is configured to operate the first member by pulling at least a portion of the first member.
17. The medical device according to any one of aspects 16, wherein the mechanical operation device comprises at least one pulley, and wherein the at least one flexible element is configured to engage the pulley such that transmission is created.
18. The medical device according to any one of aspects 1-13, wherein the first member is hydraulically operable, and the operation device is a hydraulic operation device.
19. The medical device according to aspect 18, wherein the hydraulic operation device comprises a pulling member for operating the first member.
20. The medical device according to aspect 19, wherein the pulling member comprises at least one hydraulic actuator, directly or indirectly fixated to at least one of the first and second members.
21. The medical device according to any one of aspects 18-20, wherein the medical device comprises at least one hydraulic conduit for conducting a hydraulic fluid to at least one of the hydraulic actuators.
22. The medical device according to any one of aspects 1-21, wherein at least a portion of the first member comprises elevated and lowered portions, such that the first member can move or be altered even as fibrotic tissue overgrows the first member.
23. The medical device according to aspect 22, wherein at least a portion of the first member is shaped like a bellows.
24. The medical device according to any one of the preceding aspects, wherein at least a portion of the operation device is placed in a remote unit.
25. The medical device according to aspect 24, wherein the portion of the operation device placed in the remote unit comprises at least one actuator.
26. The medical device according to aspect 25, wherein the actuator is a piezoelectric actuator.
27. The medical device according to aspect 25, wherein the actuator comprises at least one electrical motor.
28. The medical device according to aspect 27, wherein the electrical motor is a piezoelectric motor.
29. The medical device according to any one of aspects 25-28, wherein the portion of the operation device placed in the remote unit further comprises a gear system configured to reduce the speed and increase the force of the movement generated by the actuator.
30. The medical device according to aspect 29, wherein the portion of the operation device placed in the remote unit comprises at least one pulley for propelling the flexible element.
31. The medical device according to any one of aspects 27-30, wherein the portion of the operation device placed in the remote unit comprises at least one hydraulic pump.
32. The medical device according to aspect 31, wherein the hydraulic pump is a piezoelectric hydraulic pump.
33. The medical device according to any one of aspects 31 and 32, wherein the remote unit comprises a hydraulic reservoir for holding a hydraulic fluid.
34. The medical device according to aspect 33, wherein the remote unit comprises an injection port for at least one of injecting and removing hydraulic fluid from the medical device.
35. The medical device according to any one of aspects 24-34, further comprising an energy storage unit for directly or indirectly energizing the medical device.
36. The medical device according to aspect 35, wherein the energy storage unit is placed in the remote unit.
37. The medical device according to any one of aspects 24-36, further comprising a controller for controlling the operation device.
38. The medical device according to aspect 37, wherein the controller is placed in the remote unit.
39. The medical device according to any one of aspects 37-38, wherein the controller comprises a wireless transceiver for communicating wirelessly with a source external to the body of the patient.
40. The medical device according to any one of the preceding aspects, further comprising at least one sensor configured to sense a physical parameter of the medical device system.
41. The medical device according to aspect 40, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect 513—Obesity_Stretch_Version2_Lifestyle-Methods

1. A method of creating a sensation of satiety using a pre-implanted medical device configured to stretch a portion of the stomach wall of a human, the pre-implanted medical device comprises: a second member configured to be fixated to the stomach wall of the human and comprising a closed curve, a first member configured to be fixated to the stomach wall of the human within the footprint of the second member, wherein the first member is operably connected to the second member such that the first member can be displaced: relative to the second member, and within the footprint of the second member, the method comprising:
   operating the first member for displacing the first member relative to the second member within the footprint of the second member, for stretching a stomach wall portion between the first and second member, for creating a sensation of satiety.
2. The method according to aspect 1, further comprising the step of sending a wireless control signal from a wireless remote control to the pre-implanted medical device, wherein the first member is operated as a result of the receipt of the wireless control signal at the pre-implanted medical device.
3. The method according to aspect 1, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
4. A method of creating a sensation of satiety using a pre-implanted medical device configured to stretch a portion of the stomach wall of a human, the pre-implanted medical device comprises: a second member configured to be fixated to the stomach wall of the human, an operable first member configured to be fixated to the stomach wall of the human, at least partially within the footprint of the second member, wherein the first member is configured to be operated to deform, the method comprising:
   operating the first member to deform, such that a first portion of the first member is moved away from a first portion of the second member, for stretching a portion of the stomach wall between the first portion of the first member and the first portion of the second member, for creating a sensation of satiety.
5. The method according to aspect 4, further comprising the step of sending a wireless control signal from a wireless remote control to the pre-implanted medical device, wherein the first member is operated as a result of the receipt of the wireless control signal at the pre-implanted medical device.
6. The method according to aspect 4, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
7. A method of creating a sensation of satiety using a pre-implanted medical device configured to stretch a portion of the stomach wall of a human, the pre-implanted medical device comprises: an operable first member configured to be fixated to a first portion of the stomach wall, a second member configured to be fixated to a second portion of the stomach wall, and an operation, the method comprising:
   pulling at least a portion of the first member in a first direction away from at least one portion of the second member, for stretching a portion of the stomach wall between the first portion of the stomach wall and the second portion of the stomach wall for creating the sensation of satiety
8. The method according to aspect 7, further comprising the step of sending a wireless control signal from a wireless remote control to the pre-implanted medical device, wherein the first member is operated as a result of the receipt of the wireless control signal at the pre-implanted medical device.
9. The method according to aspect 7, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
10. A method of creating a sensation of satiety using a pre-implanted medical device configured to stretch a portion of the stomach wall of a human, the pre-implanted medical device comprises: an operable first member configured to be fixated to a first portion of the stomach wall, a second member configured to be fixated to a second portion of the stomach wall, a mechanical operation device configured to move at least a portion of the first member in a first direction away from at least one portion of the second member, wherein a portion of the mechanical operation device is placed in a remote unit configured to be placed at a remote location in the body of the human, the pre-implanted medical device further comprises a force transferring element configured to transfer mechanical force from the remote unit to the first member, the method comprising:
   operating the portion of the mechanical operation device placed in the remote unit such that the force transferring element transfers force from the remote unit to the operable first member for stretching a portion of the stomach wall between the first portion of the stomach wall and the second portion of the stomach wall, for creating the sensation of satiety.
11. The method according to aspect 10, further comprising the step of sending a wireless control signal from a wireless remote control to the pre-implanted medical device, wherein the portion of the mechanical operation device placed in the remote unit is operated as a result of the receipt of the wireless control signal at the pre-implanted medical device.
12. The method according to aspect 10, wherein the pre-implanted medical device further comprises a controller configured to control the operation of the portion of the mechanical operation device placed in the remote unit, and wherein the method comprises operating the portion of the mechanical operation device placed in the remote unit as a result of at least one of:
   the receipt of a wireless control signal at the controller of the pre-implanted medical device,
   the receipt of a sensor signal from a pre-implanted sensor at the controller, and
   the lapse of a pre-determined time.
13. A method of creating a sensation of satiety using a pre-implanted medical device configured to stretch a portion of the stomach wall of a human, the pre-implanted medical device comprises: an operable first member configured to be fixated to a first portion of the stomach wall, a second member configured to be fixated to a second portion of the stomach wall, an operable third member configured to be fixated to a third portion of the stomach wall, an operation device, and a mechanical switching device connected to the operation device, the method comprising:
   operating the mechanical switching device such that the mechanical switching device switches the operation device from operating the first member to move in a first direction to operating the third member to move in a third direction.
14. The method according to aspect 13, further comprising the step of sending a wireless control signal from a wireless remote control to the pre-implanted medical device, wherein the mechanical switching device is operated as a result of the receipt of the wireless control signal at the pre-implanted medical device.
15. The method according to aspect 13, wherein the pre-implanted medical device further comprises a controller configured to control the operation of the mechanical switching device, and wherein the method comprises operating the mechanical switching device as a result of at least one of:
   the receipt of a wireless control signal at the controller of the pre-implanted medical device,
   the receipt of a sensor signal from a pre-implanted sensor at the controller, and
   the lapse of a pre-determined time.
16. A method of creating a sensation of satiety using a pre-implanted medical device configured to stretch a portion of the intestinal wall of a human, the pre-implanted medical device comprises: an operable first member configured to be fixated to a first portion of the intestinal wall and an operation device connected to the operable first member, the method comprising altering the operable first member such that the intestinal wall is actively stretched for creating a sensation of satiety.
17. The method according to aspect 16, wherein the operable first member comprises at least one curved portion configured to be fixated to the first portion of the intestinal wall, and wherein the method comprises operating the operable first member for altering the curvature of the operable first member for actively stretching the first portion of the intestinal wall.
18. The method according to any one of aspects 16 and 17, wherein the operable first member is configured to be fixated to the first portion of the intestinal wall at a plurality of positions, and wherein the method comprises operating the operable first member for altering the operable first member by increasing a length of the operable first member for actively stretching the intestinal wall.
19. The method according to aspect 18, wherein the operable first member comprises a closed curve, and wherein the method comprising operating the operable first member for altering the operable first member by increasing the length of the closed curve.
20. The medical device according to aspect 16, wherein the method comprises operating the operable first member for altering the volume of the first member.
21. The method according to any one of aspects 16-20, further comprising the step of sending a wireless control signal from a wireless remote control to the pre-implanted medical device, wherein the operable first member is operated as a result of the receipt of the wireless control signal at the pre-implanted medical device.
22. The method according to any one of aspects 16-20, wherein the pre-implanted medical device further comprises a controller configured to control the operation of the operable first member, and wherein the method comprises operating the operable first member as a result of at least one of:
   the receipt of a wireless control signal at the controller of the pre-implanted medical device,
   the receipt of a sensor signal from a pre-implanted sensor at the controller, and
   the lapse of a pre-determined time.
23. A method of creating a sensation of satiety using a pre-implanted medical device configured to stretch a portion of the intestinal wall of a human, the pre-implanted medical device comprises: an operable first member configured to be fixated to a first portion of the intestinal wall, a second member configured to be fixated to a second portion of the intestinal wall, and an operation device connected to the operable first member, the method comprising operating the operable first member by moving at least a portion of the operable first member in a first direction away from the second member, for stretching the intestinal wall between the first and second portions of the intestinal wall.
24. The medical device according to aspect 20, wherein operating the operable first member by moving at least a portion of the operable first member in a first direction away from the second member comprises operating the operable first member by moving at least a portion of the operable first member in a first direction away from the second member substantially in an axial direction of the intestine.
25. The method according to any one of aspects 23-24, further comprising the step of sending a wireless control signal from a wireless remote control to the pre-implanted medical device, wherein the operable first member is operated as a result of the receipt of the wireless control signal at the pre-implanted medical device.
26. The method according to any one of aspects 23-24, wherein the pre-implanted medical device further comprises a controller configured to control the operation of the operable first member, and wherein the method comprises operating the operable first member as a result of at least one of:
   the receipt of a wireless control signal at the controller of the pre-implanted medical device,
   the receipt of a sensor signal from a pre-implanted sensor at the controller, and
   the lapse of a pre-determined time.

### Aspect 515—Obesity_Stretch_Version2_Stimulation

1. A medical device for actively stretching wall portion of a stomach or intestine of a human for creating a sensation of satiety, the medical device being implantable and comprising:
   an operable first member configured to be fixated to a first portion of the wall of the stomach or intestine, the operable first member comprising at least one of: an electrode and a vibrator configured to: electrically or mechanically stimulate a wall portion of the stomach or intestine, and
   an operation device configured to at least one of:
      move at least a portion of the first member in a first direction, and
      alter the first member, such that the wall of the stomach or intestine is actively stretched and stimulated simultaneously for creating a sensation of satiety.
2. The medical device according to aspect 1, wherein the operable first member comprises at least one curved portion configured to be fixated to the first portion of the wall of the stomach or intestine, and wherein the operation device is configured alter the curvature of the first member for actively stretching the first portion of the intestinal wall.
3. The medical device according to any one of aspects 1-2, further comprising a second member configured to be fixated to a second portion of the wall of the stomach or intestine, and wherein the operation device is configured to move at least a portion of the first member in a first direction away from the second member, for stretching the intestinal wall between the first and second portion of the stomach or intestine.
4. The medical device according to aspect 3, wherein the operation device is configured to move at least a portion of the first member in a first direction away from the second member, for stretching the wall of the stomach or intestine between the first and second portion of the stomach or intestine.
5. The medical device according to any one of the preceding aspects, wherein the operation device is configured to alter the operable first member by increasing the volume of the first member.
6. The medical device according to any one of the preceding aspects, wherein at least one of the first and second member is configured to be invaginated in the intestinal wall.
7. The medical device according to any one of the preceding aspects, wherein the first member is configured to be completely invaginated by the intestinal wall, apart from elements for transferring force or communication to or from the first member.
8. The medical device according to any one of the preceding aspects, wherein the first member is mechanically operable, and the operation device is a mechanical operation device.
9. The medical device according to aspect 8, wherein the mechanical operation device comprises a pulling member for operating the first member.
10. The medical device according to aspect 9, wherein the pulling member comprises at least one flexible element, directly or indirectly fixated to the first member, wherein the flexible element is configured to operate the first member by pulling at least a portion of the first member.
11. The medical device according to any one of aspects 1-10, wherein the first member is hydraulically operable, and the operation device is a hydraulic operation device.
12. The medical device according to aspect 11, wherein the pulling member comprises at least one hydraulic actuator, directly or indirectly fixated to at least one of the first and second members.
13. The medical device according to any one of the preceding aspects, wherein at least a portion of the operation device is placed in a remote unit.
14. The medical device according to any one of the preceding aspects, wherein the operation device is configured to pull at least a portion of the first member in a first direction away from at least one portion of the second member, for stretching a portion of the wall of the stomach or intestine between the first portion of the wall of the stomach or intestine and the second portion of the wall of the stomach or intestine for creating the sensation of satiety.
15. The medical device according to any one of the preceding aspects, wherein the first member is configured to be operated to deform such that a first portion of the first member is moved away from a first portion of the second member, for stretching a portion of the wall of the stomach or intestine between the first portion of the first member and the first portion of the second member.
16. The medical device according to any one of aspects 1-15, wherein the operation device is a piezoelectric operation device.
17. The medical device according to aspect 16, wherein the piezoelectric operation device comprises a piezoelectric motor.
18. The medical device according to any one of aspects 1-17, wherein the vibrator is configured to be operated by a piezoelectric operation device.
19. The medical device according to aspect 18, wherein the piezoelectric operation device comprises a piezoelectric motor.
20. The medical device according to aspect 19, wherein the piezoelectric motor is a piezoelectric ultrasonic motor.
21. The medical device according to any one of aspects 16-20, wherein the piezoelectric operation device is substantially non-magnetic.
22. The medical device according to any one of aspects 16-21, wherein the piezoelectric operation device is substantially non-metallic.
23. The medical device according to any one of aspects 1-22, wherein the electrode or vibrator are connected to a wireless energy receiver configured to receive energy for powering the electrode or vibrator wirelessly.
23. The medical device according to any one of aspects 1-22, wherein the operable first member including the electrode or vibrator is configured to be at least partially invaginated by the tissue of the stomach wall using stomach-to-stomach sutures or staplers.
24. The medical device according to any one of aspects 1-23, wherein the vibrator is configured to vibrate at a frequency in the range of 1-100 Hz, such as in the range of 1-20 Hz.
25. The medical device according to any one of aspects 1-24, wherein the vibrator is configured to vibrate with a period of 0.01-1 seconds, such as of 0.05-1 seconds.
26. The medical device according to any one of aspects 1-25, wherein the vibrator is configured to vibrate at an amplitude such that the tissue in the stomach/intestine wall is displaced at least 1 mm.
27. The medical device according to any one of aspects 1-17, wherein the medical device further comprises a second electrode, and wherein the first and second electrodes are configured for successively electrically stimulating two portions of the tissue wall of the stomach or intestine.
28. The medical device according to aspect 27, wherein the first and second electrode are placed on the operable first member at a distance from each other being at least 5 mm.
29. The medical device according to aspect 27, wherein the first and second electrode are placed on the operable first member at a distance from each other corresponding to a wavelength of a wave pattern of a movement of the tissue wall of the stomach or intestine, or by a multiple of such a wavelength.
30. The medical device according to any one of aspects 27-29, further comprising a stimulation controller configured to send stimulation signals to the first and second electrode in pulses, or pulse trains.
31. The medical device according to aspect 30, wherein the stimulation controller is configured to send stimulation signals to the first and second electrode in pulses or pulse trains having a frequency being determined based on a frequency of the wave pattern of a movement of smooth muscles of the stomach or intestine.
31. The medical device according to aspect 30, wherein the stimulation controller is configured to send stimulation signals to the first and second electrode in pulses or pulse trains having a pulse separation or pulse train separation of 0,5-10 s, such as 1-8 s, such as 2-6 s.

### AspectS P—Obesity-Stretch-Version2-P

### Aspect P1—Obesity Stretch Version2 Circumferance

1. A medical device for actively stretching a stomach or intestinal wall of a human for creating a sensation of satiety, the medical device being implantable and comprising:
   an operable first member configured to be directly or indirectly fixated to a first portion of the stomach or intestinal wall, wherein the operable first member is configured to be invaginated and held in place by tissue-to-tissue sutures or staplers,
   a second member configured to be directly or indirectly fixated to a second portion of the stomach or intestinal wall, wherein the second member is configured to be invaginated and held in place by tissue-to-tissue sutures or staplers,
   an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane exceeds ½ of the length of a circumference of the portion of the operable first member positioned in the stretching plane.
2. The medical device according to claim 1, wherein the portion of the operable first member positioned in the stretching plane is configured to be invaginated.
3. The medical device according to any one of claims 1 and 2, wherein the operation device is configured to move at least a portion of the operable first member by deforming the operable first member.
4. The medical device according to any one of the preceding claims, wherein the operation device is configured to move at least a portion of the operable first member by pulling at least a portion of the first member in a first direction away from at least one portion of the second member.
5. The medical device according to any one of the preceding claims, wherein at least a portion of the operation device is placed in a remote unit configured to be placed at a remote location in the body of the patient.
6. The medical device according to any one of the preceding aspects, wherein the operable first member is mechanically operable, and the operation device is a mechanical operation device.
7. The medical device according to aspect 6, wherein the mechanical operation device comprises a pulling member for operating the operable first member.
8. The medical device according to aspect 7, wherein the pulling member comprises at least one flexible element, directly or indirectly fixated to the first member, wherein the flexible element is configured to operate the first member by pulling at least a portion of the first member.
9. The medical device according to any one of aspects 1-5, wherein the operable first member is hydraulically operable, and the operation device is a hydraulic operation device.
10. The medical device according to aspect 9, wherein the pulling member comprises at least one hydraulic actuator, directly or indirectly fixated to at least one of the first and second members.
11. The medical device according to any one of the preceding aspects, wherein the operation device is a piezoelectric operation device.
12. The medical device according to aspect 11, wherein the piezoelectric operation device comprises a piezoelectric motor.
13. The medical device according to any one of the preceding aspects, further comprising a controller for controlling the operation device.
14. The medical device according to aspect 13, wherein the controller is placed in the remote unit.
15. The medical device according to any one of aspects 13-14, wherein the controller comprises a wireless transceiver for communicating wirelessly with a source external to the body of the patient.
16. The medical device according to any one of the preceding aspects, further comprising at least one sensor configured to sense a physical parameter of the medical device system.
17. The medical device according to aspect 16, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P1—Obesity Stretch Version2 Circumferance US Method

1. A method of implanting a medical device configured to stretch a portion of the stomach or intestinal wall of a human, the medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, and an operation device for operating the first member for displacing the first member relative to the second member, the method comprising invaginating the first member at a first position in the stomach or intestinal wall and invaginating the second member at a second position in the stomach or intestinal, such that a stretching plane extends between the first and second members in which plane a portion of the operable first member is positioned, and such that a length of a distance between the first and second member in the stretching plane exceeds ½ of the length of a circumference of the portion of the operable first member positioned in the stretching plane.
2. The method according to claim 1, further comprising the step of placing an operation device configured for moving at least a portion of the operable first member by deforming the operable first member.
3. The method according to any one of the preceding claims, further comprising the step of placing an operation device configured for displacing the first member relative to the second member by pulling at least a portion of the first member in a first direction away from at least one portion of the second member.
4. The method according to any one of the preceding aspects, further comprising the step of placing an implantable sensor wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
5. The method according to any one of the preceding aspects, wherein the method is a laparoscopic method comprising at least one of the steps of:
   introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,
   blowing in pressurized gas through the needle or trocar or surgical instrument or any device inflating the abdominal cavity,
   introducing working instruments comprising at least one of the steps of,
      inserting at least one first trocar into the abdominal cavity,
      introducing at least one camera through the trocar,
      inserting at least a second trocar into the abdominal cavity,
      inserting at least one instrument preferably through the second trocar,
      inserting at least a third trocar into the abdominal cavity,
      inserting at least one second instrument preferably through the third trocar,
   dissecting an area of the stomach or intestine,
   identifying a suitable position for placing a stretching device on the stomach or intestinal wall,
   placing a control device in the body for controlling the stretching device,
   inserting the medical device into the abdomen of the patient,
   fixating the operable first member to a first portion of the stomach or intestinal wall,
   fixating the second member to a second portion of the stomach or intestinal wall,
   placing the control unit, and
   extracting the instruments, camera and trocar, and in relation thereto suturing, if necessary, the abdominal wall and permanently closing the skin.
6. The method according to any one of aspects 1-4, wherein the method is a surgical method comprising at least one of the steps of:
   cutting and open the skin and abdominal wall.
dissecting or cutting through the fascia of at least one of any muscles or any fibrotic intersections
   comprising; the rectus abdominalis, transversus abdominalis, the External Oblique, the Internal Oblique, the linea alba, a tendnous intersection, and the umbilicus,
   introducing at least one working instrument,
   dissecting an area of the stomach,
   identifying a suitable position for placing a stretching device on the stomach wall,
   placing a control device in the body for controlling the stretching device,
   inserting the medical device into the abdomen of the patient,
   fixating the operable first member to a first portion of the stomach wall,
   fixating the second member to a second portion of the stomach wall,
   placing the control unit, and
   extracting the instruments, camera and trocar, and in relation thereto suturing, if necessary, the abdominal wall and permanently closing the skin.

### Aspect P1—Obesity Stretch Version2 Circumferance EP Method

1. A method of creating a sensation of satiety using a pre-implanted medical device configured to stretch a portion of the stomach or intestinal wall of a human, the pre-implanted medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane exceeds ½ of the length of a circumference of the portion of the operable first member positioned in the stretching plane. the method comprising:
operating the operable first member by moving at least a portion of the operable first member in a first direction away from the second member, for stretching the stomach or intestinal wall between the first and second portions of the stomach or intestinal wall.
2. The method according to aspect 1, further comprising the step of sending a wireless control signal from a wireless remote control to the pre-implanted medical device, wherein the first member is operated as a result of the receipt of the wireless control signal at the pre-implanted medical device.
3. The method according to aspect 1, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
4. The method according to any one of claims 1-3, wherein the step of operating the first member for displacing the first member relative to the second member comprises moving at least a portion of the operable first member by deforming the operable first member.
5. The method according to any one of the preceding claims, wherein the step of operating the first member for displacing the first member relative to the second member comprises moving at least a portion of the operable first member by pulling at least a portion of the first member in a first direction away from at least one portion of the second member.
6. The method according to aspect 3, wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P2—Obesity Stretch Version2 Circumferance 2

1. A medical device for actively stretching a stomach or intestinal wall of a human for creating a sensation of satiety, the medical device being implantable and comprising:
   an operable first member configured to be directly or indirectly fixated to a first portion of the stomach or intestinal wall, wherein the operable first member is configured to be invaginated and held in place by tissue-to-tissue sutures or staplers,
   a second member configured to be directly or indirectly fixated to a second portion of the stomach or intestinal wall, wherein the second member is configured to be invaginated and held in place by tissue-to-tissue sutures or staplers,
   an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane is less than ½ of the length of a circumference of the portion of the operable first member positioned in the stretching plane.
2. The medical device according to claim 1, wherein the portion of the operable first member positioned in the stretching plane is configured to be invaginated.
3. The medical device according to any one of claims 1 and 2, wherein the operation device is configured to move at least a portion of the operable first member by deforming the operable first member.
4. The medical device according to any one of the preceding claims, wherein the operation device is configured to move at least a portion of the operable first member by pulling at least a portion of the first member in a first direction away from at least one portion of the second member.
5. The medical device according to any one of the preceding claims, wherein at least a portion of the operation device is placed in a remote unit configured to be placed at a remote location in the body of the patient.
6. The medical device according to any one of the preceding aspects, wherein the operable first member is mechanically operable, and the operation device is a mechanical operation device.
7. The medical device according to aspect 6, wherein the mechanical operation device comprises a pulling member for operating the operable first member.
8. The medical device according to aspect 7, wherein the pulling member comprises at least one flexible element, directly or indirectly fixated to the first member, wherein the flexible element is configured to operate the first member by pulling at least a portion of the first member.
9. The medical device according to any one of aspects 1-5, wherein the operable first member is hydraulically operable, and the operation device is a hydraulic operation device.
10. The medical device according to aspect 9, wherein the pulling member comprises at least one hydraulic actuator, directly or indirectly fixated to at least one of the first and second members.
11. The medical device according to any one of the preceding aspects, wherein the operation device is a piezoelectric operation device.
12. The medical device according to aspect 11, wherein the piezoelectric operation device comprises a piezoelectric motor.
13. The medical device according to any one of the preceding aspects, further comprising a controller for controlling the operation device.
14. The medical device according to aspect 13, wherein the controller is placed in the remote unit.
15. The medical device according to any one of aspects 13-14, wherein the controller comprises a wireless transceiver for communicating wirelessly with a source external to the body of the patient.
16. The medical device according to any one of the preceding aspects, further comprising at least one sensor configured to sense a physical parameter of the medical device system.
17. The medical device according to aspect 16, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P2—Obesity Stretch Version2 Circumferance 2 US Method

1. A method of implanting a medical device configured to stretch a portion of the stomach or intestinal wall of a human, the medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall,
wherein the second member is configured to be invaginated and held in place by tissue-to-tissue sutures or staplers, and an operation device for operating the first member for displacing the first member relative to the second member, the method comprising invaginating the first member at a first position in the stomach or intestinal wall and invaginating the second member at a second position in the stomach or intestinal wall, such that a stretching plane extends between the first and second members in which plane a portion of the operable first member is positioned, and such that a length of a distance between the first and second member in the stretching plane is less than ½ of the length of a circumference of the portion of the operable first member positioned in the stretching plane.
2. The method according to claim 1, further comprising the step of placing an operation device configured for moving at least a portion of the operable first member by deforming the operable first member.
3. The method according to any one of the preceding claims, further comprising the step of placing an operation device configured for displacing the first member relative to the second member by pulling at least a portion of the first member in a first direction away from at least one portion of the second member.
4. The method according to any one of the preceding aspects, further comprising the step of placing an implantable sensor wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
5. The method according to any one of the preceding aspects, wherein the method is a laparoscopic method comprising at least one of the steps of:
   introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,
   blowing in pressurized gas through the needle or trocar or surgical instrument or any device inflating the abdominal cavity,
   introducing working instruments comprising at least one of the steps of,
      inserting at least one first trocar into the abdominal cavity,
      introducing at least one camera through the trocar,
      inserting at least a second trocar into the abdominal cavity,
      inserting at least one instrument preferably through the second trocar,
      inserting at least a third trocar into the abdominal cavity,
      inserting at least one second instrument preferably through the third trocar,
   dissecting an area of the stomach or intestine,
   identifying a suitable position for placing a stretching device on the stomach or intestinal wall,
   placing a control device in the body for controlling the stretching device,
   inserting the medical device into the abdomen of the patient,
   fixating the operable first member to a first portion of the stomach or intestinal wall,
   fixating the second member to a second portion of the stomach or intestinal wall,
   placing the control unit, and
   extracting the instruments, camera and trocar, and in relation thereto suturing, if necessary, the abdominal wall and permanently closing the skin.
6. The method according to any one of aspects 1-4, wherein the method is a surgical method comprising at least one of the steps of:
   cutting and open the skin and abdominal wall.
dissecting or cutting through the fascia of at least one of any muscles or any fibrotic intersections
   comprising; the rectus abdominalis, transversus abdominalis, the External Oblique, the Internal Oblique, the linea alba, a tendnous intersection, and the umbilicus,
   introducing at least one working instrument,
   dissecting an area of the stomach,
   identifying a suitable position for placing a stretching device on the stomach wall,
   placing a control device in the body for controlling the stretching device,
   inserting the medical device into the abdomen of the patient,
   fixating the operable first member to a first portion of the stomach wall,
   fixating the second member to a second portion of the stomach wall,
   placing the control unit, and
   extracting the instruments, camera and trocar, and in relation thereto suturing, if necessary, the abdominal wall and permanently closing the skin.

### Aspect P2—Obesity Stretch Version2 Circumferance 2 EP Method

1. A method of creating a sensation of satiety using a pre-implanted medical device configured to stretch a portion of the stomach or intestinal wall of a human, the pre-implanted medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein a length of a distance between the first and second member in the stretching plane is less than ½ of the length of a circumference of the portion of the operable first member positioned in the stretching plane. the method comprising: operating the operable first member by moving at least a portion of the operable first member in a first direction away from the second member, for stretching the stomach or intestinal wall between the first and second portions of the stomach or intestinal wall.
2. The method according to aspect 1, further comprising the step of sending a wireless control signal from a wireless remote control to the pre-implanted medical device, wherein the first member is operated as a result of the receipt of the wireless control signal at the pre-implanted medical device.
3. The method according to aspect 1, wherein the pre-implanted medical device further comprises a controller configured to control the operation of the first member, and wherein the method comprises operating the first member as a result of at least one of:
the receipt of a wireless control signal at the controller of the pre-implanted medical device,
the receipt of a sensor signal from a pre-implanted sensor at the controller, and
the lapse of a pre-determined time.
4. The method according to any one of claims 1-3, wherein the step of operating the first member for displacing the first member relative to the second member comprises moving at least a portion of the operable first member by deforming the operable first member.
5. The method according to any one of the preceding claims, wherein the step of operating the first member for displacing the first member relative to the second member comprises moving at least a portion of the operable first member by pulling at least a portion of the first member in a first direction away from at least one portion of the second member.
6. The method according to aspect 3, wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P3—Obesity Stretch Version2 Cross-sectional Area

1. A medical device for actively stretching a stomach or intestinal wall of a human for creating a sensation of satiety, the medical device being implantable and comprising:
   an operable first member configured to be directly or indirectly fixated to a first portion of the stomach or intestinal wall, wherein the operable first member is configured to be invaginated and held in place by tissue-to-tissue sutures or staplers,
   a second member configured to be directly or indirectly fixated to a second portion of the stomach or intestinal wall, wherein the second member is configured to be invaginated and held in place by tissue-to-tissue sutures or staplers,
   an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member exceeds an area of the portion of the operable first member positioned in the stretching plane.
2. The medical device according to claim 1, wherein the portion of the operable first member positioned in the stretching plane is configured to be invaginated.
3. The medical device according to any one of claims 1 and 2, wherein the operation device is configured to move at least a portion of the operable first member by deforming the operable first member.
4. The medical device according to any one of the preceding claims, wherein the operation device is configured to move at least a portion of the operable first member by pulling at least a portion of the first member in a first direction away from at least one portion of the second member.
5. The medical device according to any one of the preceding claims, wherein at least a portion of the operation device is placed in a remote unit configured to be placed at a remote location in the body of the patient.
6. The medical device according to any one of the preceding aspects, wherein the operable first member is mechanically operable, and the operation device is a mechanical operation device.
7. The medical device according to aspect 6, wherein the mechanical operation device comprises a pulling member for operating the operable first member.
8. The medical device according to aspect 7, wherein the pulling member comprises at least one flexible element, directly or indirectly fixated to the first member, wherein the flexible element is configured to operate the first member by pulling at least a portion of the first member.
9. The medical device according to any one of aspects 1-5, wherein the operable first member is hydraulically operable, and the operation device is a hydraulic operation device.
10. The medical device according to aspect 9, wherein the pulling member comprises at least one hydraulic actuator, directly or indirectly fixated to at least one of the first and second members.
11. The medical device according to any one of the preceding aspects, wherein the operation device is a piezoelectric operation device.
12. The medical device according to aspect 11, wherein the piezoelectric operation device comprises a piezoelectric motor.
13. The medical device according to any one of the preceding aspects, further comprising a controller for controlling the operation device.
14. The medical device according to aspect 13, wherein the controller is placed in the remote unit.
15. The medical device according to any one of aspects 13-14, wherein the controller comprises a wireless transceiver for communicating wirelessly with a source external to the body of the patient.
16. The medical device according to any one of the preceding aspects, further comprising at least one sensor configured to sense a physical parameter of the medical device system.
17. The medical device according to aspect 16, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P3—Obesity Stretch Version2 Cross-sectional Area US Method

1. A method of implanting a medical device configured to stretch a portion of the stomach or intestinal wall of a human, the medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, and an operation device for operating the first member for displacing the first member relative to the second member, the method comprising invaginating the first member at a first position in the stomach or intestinal wall and invaginating the second member at a second position in the stomach or intestinal wall, such that a stretching plane extends between the first and second members in which plane a portion of the operable first member is positioned, and such that an area of the stretching plane between the first and second member exceeds an area of the portion of the operable first member positioned in the stretching plane.
2. The method according to claim 1, further comprising the step of placing an operation device configured for moving at least a portion of the operable first member by deforming the operable first member.
3. The method according to any one of the preceding claims, further comprising the step of placing an operation device configured for displacing the first member relative to the second member by pulling at least a portion of the first member in a first direction away from at least one portion of the second member.
4. The method according to any one of the preceding aspects, further comprising the step of placing an implantable sensor wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
5. The method according to any one of the preceding aspects, wherein the method is a laparoscopic method comprising at least one of the steps of:
   introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,
   blowing in pressurized gas through the needle or trocar or surgical instrument or any device inflating the abdominal cavity,
   introducing working instruments comprising at least one of the steps of,
      inserting at least one first trocar into the abdominal cavity,
      introducing at least one camera through the trocar,
      inserting at least a second trocar into the abdominal cavity,
      inserting at least one instrument preferably through the second trocar,
      inserting at least a third trocar into the abdominal cavity,
      inserting at least one second instrument preferably through the third trocar,
   dissecting an area of the stomach or intestine,
   identifying a suitable position for placing a stretching device on the stomach or intestinal wall,
   placing a control device in the body for controlling the stretching device,
   inserting the medical device into the abdomen of the patient,
   fixating the operable first member to a first portion of the stomach or intestinal wall,
   fixating the second member to a second portion of the stomach or intestinal wall,
   placing the control unit, and
   extracting the instruments, camera and trocar, and in relation thereto suturing, if necessary, the abdominal wall and permanently closing the skin.
6. The method according to any one of aspects 1-4, wherein the method is a surgical method comprising at least one of the steps of:
   cutting and open the skin and abdominal wall.
   dissecting or cutting through the fascia of at least one of any muscles or any fibrotic intersections comprising; the rectus abdominalis, transversus abdominalis, the External Oblique, the Internal Oblique, the linea alba, a tendnous intersection, and the umbilicus,
   introducing at least one working instrument,
   dissecting an area of the stomach,
   identifying a suitable position for placing a stretching device on the stomach wall,
   placing a control device in the body for controlling the stretching device,
   inserting the medical device into the abdomen of the patient,
   fixating the operable first member to a first portion of the stomach wall,
   fixating the second member to a second portion of the stomach wall,
   placing the control unit, and
   extracting the instruments, camera and trocar, and in relation thereto suturing, if necessary, the abdominal wall and permanently closing the skin.

### Aspect P3—Obesity Stretch Version2 Cross-secotional Area EP Method

1. A method of creating a sensation of satiety using a pre-implanted medical device configured to stretch a portion of the stomach or intestinal wall of a human, the pre-implanted medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member exceeds an area of the portion of the operable first member positioned in the stretching plane, the method comprising:
   operating the operable first member by moving at least a portion of the operable first member in a first direction away from the second member, for stretching the stomach or intestinal wall between the first and second portions of the stomach or intestinal wall.
2. The method according to aspect 1, further comprising the step of sending a wireless control signal from a wireless remote control to the pre-implanted medical device, wherein the first member is operated as a result of the receipt of the wireless control signal at the pre-implanted medical device.
3. The method according to aspect 1, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
4. The method according to any one of claims 1-3, wherein the step of operating the first member for displacing the first member relative to the second member comprises moving at least a portion of the operable first member by deforming the operable first member.
5. The method according to any one of the preceding claims, wherein the step of operating the first member for displacing the first member relative to the second member comprises moving at least a portion of the operable first member by pulling at least a portion of the first member in a first direction away from at least one portion of the second member.
6. The method according to aspect 3, wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient, a hydraulic pressure in at least one of the hydraulic actuator, the hydraulic pump, the hydraulic reservoir and the hydraulic conduit.

### Aspect P4—Obesity Stretch Version2 Cross-sectional Area 2

1. A medical device for actively stretching a stomach or intestinal wall of a human for creating a sensation of satiety, the medical device being implantable and comprising:
   an operable first member configured to be directly or indirectly fixated to a first portion of the stomach or intestinal wall, wherein the operable first member is configured to be invaginated and held in place by tissue-to-tissue sutures or staplers,
   a second member configured to be directly or indirectly fixated to a second portion of the stomach or intestinal wall, wherein the second member is configured to be invaginated and held in place by tissue-to-tissue sutures or staplers,
   an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member is less than an area of the portion of the operable first member positioned in the stretching plane.
2. The medical device according to claim 1, wherein the portion of the operable first member positioned in the stretching plane is configured to be invaginated.
3. The medical device according to any one of claims 1 and 2, wherein the operation device is configured to move at least a portion of the operable first member by deforming the operable first member.
4. The medical device according to any one of the preceding claims, wherein the operation device is configured to move at least a portion of the operable first member by pulling at least a portion of the first member in a first direction away from at least one portion of the second member.
5. The medical device according to any one of the preceding claims, wherein at least a portion of the operation device is placed in a remote unit configured to be placed at a remote location in the body of the patient.
6. The medical device according to any one of the preceding aspects, wherein the operable first member is mechanically operable, and the operation device is a mechanical operation device.
7. The medical device according to aspect 6, wherein the mechanical operation device comprises a pulling member for operating the operable first member.
8. The medical device according to aspect 7, wherein the pulling member comprises at least one flexible element, directly or indirectly fixated to the first member, wherein the flexible element is configured to operate the first member by pulling at least a portion of the first member.
9. The medical device according to any one of aspects 1-5, wherein the operable first member is hydraulically operable, and the operation device is a hydraulic operation device.
10. The medical device according to aspect 9, wherein the pulling member comprises at least one hydraulic actuator, directly or indirectly fixated to at least one of the first and second members.
11. The medical device according to any one of the preceding aspects, wherein the operation device is a piezoelectric operation device.
12. The medical device according to aspect 11, wherein the piezoelectric operation device comprises a piezoelectric motor.
13. The medical device according to any one of the preceding aspects, further comprising a controller for controlling the operation device.
14. The medical device according to aspect 13, wherein the controller is placed in the remote unit.
15. The medical device according to any one of aspects 13-14, wherein the controller comprises a wireless transceiver for communicating wirelessly with a source external to the body of the patient.
16. The medical device according to any one of the preceding aspects, further comprising at least one sensor configured to sense a physical parameter of the medical device system.
17. The medical device according to aspect 16, wherein the sensor is a sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P4—Obesity Stretch Version2 Cross-secotional Area 2 US Method

1. A method of implanting a medical device configured to stretch a portion of the stomach or intestinal wall of a human, the medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, and an operation device for operating the first member for displacing the first member relative to the second member, the method comprising invaginating the first member at a first position in the stomach or intestinal wall and invaginating the second member at a second position in the stomach or intestinal wall, such that a stretching plane extends between the first and second members in which plane a portion of the operable first member is positioned, and such that an area of the stretching plane between the first and second member is less than an area of the portion of the operable first member positioned in the stretching plane.
2. The method according to claim 1, further comprising the step of placing an operation device configured for moving at least a portion of the operable first member by deforming the operable first member.
3. The method according to any one of the preceding claims, further comprising the step of placing an operation device configured for displacing the first member relative to the second member by pulling at least a portion of the first member in a first direction away from at least one portion of the second member.
4. The method according to any one of the preceding aspects, further comprising the step of placing an implantable sensor wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
5. The method according to any one of the preceding aspects, wherein the method is a laparoscopic method comprising at least one of the steps of:
   introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,
   blowing in pressurized gas through the needle or trocar or surgical instrument or any device inflating the abdominal cavity,
   introducing working instruments comprising at least one of the steps of,
      inserting at least one first trocar into the abdominal cavity,
      introducing at least one camera through the trocar,
      inserting at least a second trocar into the abdominal cavity,
      inserting at least one instrument preferably through the second trocar,
      inserting at least a third trocar into the abdominal cavity,
      inserting at least one second instrument preferably through the third trocar,
   dissecting an area of the stomach or intestine,
   identifying a suitable position for placing a stretching device on the stomach or intestinal wall,
   placing a control device in the body for controlling the stretching device,
   inserting the medical device into the abdomen of the patient,
   fixating the operable first member to a first portion of the stomach or intestinal wall,
   fixating the second member to a second portion of the stomach or intestinal wall,
   placing the control unit, and
   extracting the instruments, camera and trocar, and in relation thereto suturing, if necessary, the abdominal wall and permanently closing the skin.
6. The method according to any one of aspects 1-4, wherein the method is a surgical method comprising at least one of the steps of:
   cutting and open the skin and abdominal wall.
   dissecting or cutting through the fascia of at least one of any muscles or any fibrotic intersections comprising; the rectus abdominalis, transversus abdominalis, the External Oblique, the Internal Oblique, the linea alba, a tendnous intersection, and the umbilicus,
   introducing at least one working instrument,
   dissecting an area of the stomach,
   identifying a suitable position for placing a stretching device on the stomach wall,
   placing a control device in the body for controlling the stretching device,
   inserting the medical device into the abdomen of the patient,
   fixating the operable first member to a first portion of the stomach wall,
   fixating the second member to a second portion of the stomach wall,
   placing the control unit, and
   extracting the instruments, camera and trocar, and in relation thereto suturing, if necessary, the abdominal wall and permanently closing the skin.

### Aspect P4—Obesity Stretch Version2 Cross-secotional Area 2 EP Method

1. A method of creating a sensation of satiety using a pre-implanted medical device configured to stretch a portion of the stomach or intestinal wall of a human, the pre-implanted medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, an operation device configured to move at least a portion of the operable first member in a first direction away from the second member, for stretching the stomach or intestinal wall at least in a stretching plane extending between the first and second member, wherein a portion of the operable first member is positioned in the stretching plane, and wherein an area of the stretching plane between the first and second member is less than an area of the portion of the operable first member positioned in the stretching plane, the method comprising:
   operating the operable first member by moving at least a portion of the operable first member in a first direction away from the second member, for stretching the intestinal wall between the first and second portions of the stomach or intestinal wall.
2. The method according to aspect 1, further comprising the step of sending a wireless control signal from a wireless remote control to the pre-implanted medical device, wherein the first member is operated as a result of the receipt of the wireless control signal at the pre-implanted medical device.
3. The method according to aspect 1, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
4. The method according to any one of claims 1-3, wherein the step of operating the first member for displacing the first member relative to the second member comprises moving at least a portion of the operable first member by deforming the operable first member.
5. The method according to any one of the preceding claims, wherein the step of operating the first member for displacing the first member relative to the second member comprises moving at least a portion of the operable first member by pulling at least a portion of the first member in a first direction away from at least one portion of the second member.
6. The method according to aspect 3, wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P5—Obesity Stretch Version2 Method Undisected EP

1. A method of creating a sensation of satiety using a pre-implanted medical device configured to stretch a portion of the stomach or intestinal wall of a human, the pre-implanted medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, the method comprising:
   operating the first member for displacing the first member relative to the second member for stretching a stomach or intestinal wall portion between the first and second member which is undissected and devoid of any implanted parts, to avoid any important fibrotic tissue interfering with the stretching of the stomach or intestinal wall.
2. The method according to aspect 1, further comprising the step of sending a wireless control signal from a wireless remote control to the pre-implanted medical device, wherein the first member is operated as a result of the receipt of the wireless control signal at the pre-implanted medical device.
3. The method according to aspect 1, wherein the pre-implanted medical device further comprises a controller configured to control the operation of the first member, and wherein the method comprises operating the first member as a result of at least one of:
   the receipt of a wireless control signal at the controller of the pre-implanted medical device,
   the receipt of a sensor signal from a pre-implanted sensor at the controller, and
   the lapse of a pre-determined time.
4. The method according to any one of claims 1-3, wherein the step of operating the first member for displacing the first member relative to the second member comprises moving at least a portion of the operable first member by deforming the operable first member.
5. The method according to any one of the preceding claims, wherein the step of operating the first member for displacing the first member relative to the second member comprises moving at least a portion of the operable first member by pulling at least a portion of the first member in a first direction away from at least one portion of the second member.
6. The method according to aspect 3, wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.

### Aspect P5—Obesity Stretch Version2 Method Undisected US

1. A method of implanting a medical device configured to stretch a portion of the stomach or intestinal wall of a human, the medical device comprises: an operable first member configured to be directly or indirectly fixated to a first portion of the stomach or intestinal wall, wherein the operable first member is configured to be invaginated and held in place by tissue-to-tissue sutures or staplers, a second member configured to be directly or indirectly fixated to a second portion of the stomach or intestinal wall, wherein the second member is configured to be invaginated and held in place by tissue-to-tissue sutures or staplers, and an operation device for operating the first member for displacing the first member relative to the second member, the method comprising invaginating the first member at a first position in the stomach or intestinal wall and invaginating the second member at a second position in the stomach or intestinal, such that a stomach or intestinal wall portion between the first and second member is undissected and devoid of any implanted parts, such that the stomach or intestinal wall portion between the first and second member can be stretched without interference from fibrotic tissue.
2. The method according to claim 1, further comprising the step of placing an operation device configured for moving at least a portion of the operable first member by deforming the operable first member.
3. The method according to any one of the preceding claims, further comprising the step of placing an operation device configured for displacing the first member relative to the second member by pulling at least a portion of the first member in a first direction away from at least one portion of the second member.
4. The method according to any one of the preceding aspects, further comprising the step of placing an implantable sensor wherein the method comprises operating the first member as a result of the receipt of a sensor signal from a pre-implanted sensor configured to sense at least one of:
   a temperature of the medical device system,
   a parameter related to the power consumption of the medical device system,
   a parameter related to strain in at least one of the first and second members,
   a parameter related to a status of the energy storage unit,
   a parameter related to the wireless transfer of energy from a source external to the body of the patient,
   a hydraulic pressure in at least one of the hydraulic actuator, the hydraulic pump, the hydraulic reservoir and the hydraulic conduit.
5. The method according to any one of the preceding aspects, wherein the method is a laparoscopic method comprising at least one of the steps of:
   introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, the linea alba, a tendnous intersection, and the umbilicus,
   blowing in pressurized gas through the needle or trocar or surgical instrument or any device inflating the abdominal cavity,
   introducing working instruments comprising at least one of the steps of,
      inserting at least one first trocar into the abdominal cavity,
      introducing at least one camera through the trocar,
      inserting at least a second trocar into the abdominal cavity,
      inserting at least one instrument preferably through the second trocar,
      inserting at least a third trocar into the abdominal cavity,
      inserting at least one second instrument preferably through the third trocar,
   dissecting an area of the stomach or intestine,
   identifying a suitable position for placing a stretching device on the stomach or intestinal wall,
   placing a control device in the body for controlling the stretching device,
   inserting the medical device into the abdomen of the patient,
   fixating the operable first member to a first portion of the stomach or intestinal wall,
   fixating the second member to a second portion of the stomach or intestinal wall,
   placing the control unit, and
   extracting the instruments, camera and trocar, and in relation thereto suturing, if necessary, the abdominal wall and permanently closing the skin.
6. The method according to any one of aspects 1-4, wherein the method is a surgical method comprising at least one of the steps of:
   cutting and open the skin and abdominal wall.
   dissecting or cutting through the fascia of at least one of any muscles or any fibrotic intersections comprising; the rectus abdominalis, transversus abdominalis, the External Oblique, the Internal Oblique, the linea alba, a tendnous intersection, and the umbilicus,
   introducing at least one working instrument,
   dissecting an area of the stomach,
   identifying a suitable position for placing a stretching device on the stomach wall,
   placing a control device in the body for controlling the stretching device,
   inserting the medical device into the abdomen of the patient,
   fixating the operable first member to a first portion of the stomach wall,
   fixating the second member to a second portion of the stomach wall,
   placing the control unit, and
   extracting the instruments, camera and trocar, and in relation thereto suturing, if necessary, the abdominal wall and permanently closing the skin.

### Aspect 448—Operation_General_Bowden_Switch

1. An implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
2. The implantable operation device according to aspect 1, wherein the shaft is configured to transfer a rotational force when the switch is in the first state and transfer an axial force when the switch is in the second state.
3. The implantable operation device according to any one of aspects 1-2, wherein the shaft comprises an engaging portion, and wherein the electrical machine is configured to move the shaft in an axial direction, such that the engaging portion can move from a first position to a second position, and wherein the engaging portion:
   in the first position operatively engages a first operating element, and
   in the second position does not operatively engage the first operating element.
4. The implantable operation device according to any one of aspects 1-3, wherein the shaft comprises an engaging portion, and wherein the electrical machine is configured to move the shaft in an axial direction, such that the engaging portion can move from a first position to a second position, and wherein the engaging portion:
   in the first position operatively engages a first operating element, and
   in the second position operatively engages a second operating element.
5. The implantable operation device according to any one of aspects 1-4, wherein the shaft comprises at least one flexible portion.
6. The implantable operation device according to any one of aspects 1-5, further comprising at least one sheath configured to house at least a portion of the shaft.
7. The implantable operation device according to aspect 6, wherein the sheath is a flexible sheath.
8. The implantable operation device according to any one of aspects 6 and 7, wherein the sheath is configured to travel from the operation device to the body engaging portion of the medical implant.
9. The implantable operation device according to aspect 8, wherein the sheath comprises elevated and lowered portions, such that the sheath can remain flexible even as fibrotic tissue overgrows the sheath.
10. The implantable operation device according to any one of the preceding aspects, wherein the switch is an electrically powered switch.
11. The implantable operation device according to any one of the preceding aspects, wherein the switch is operable by the electrical machine.
12. The implantable operation device according to any one of the preceding aspects, wherein the electrical machine is an electrical motor.
13. The implantable operation device according to any one of aspects 1-10, further comprising an additional electrical motor or a solenoid configured to operate the switch.
14. The implantable operation device according to any one of aspects 1-9, wherein the switch is operable by centrifugal force.
15. The implantable operation device according to aspect 14, wherein the switch comprises a force output element operable by centrifugal force, such that the force output element:
   when rotated at a first speed engages a first force receiving element, and
   when rotated at a second speed engages a second force receiving element.
16. The implantable operation device according to aspect 15, wherein:
   the first force receiving element is connected to the shaft such that rotation of the first force receiving element causes rotation in the shaft, and
   the second force receiving element comprises the transmission configured to transform the rotating force into a linear force, such that rotation of the first force receiving element causes linear movement of the shaft.
17. The implantable operation device according to any one of the preceding aspects, further comprising a speed controller configured to control the speed of the electrical motor.
18. The implantable operation device according to aspect 17, wherein the speed controller is configured to control the speed of the electrical motor, such that the electrical motor, at a first speed, engages the first force receiving element, at a second speed engages the second force receiving element.
19. The implantable operation device according to any one of the preceding aspects, wherein the implantable operation device is configured to be placed at least partially in a remote unit.
20. The implantable operation device according to any one of the preceding aspects, wherein the shaft comprises a Bowden cable.
21. The implantable operation device according to any one of the preceding aspects, further comprising a pre-tensioning device for creating a pre-tension in the shaft.
22. The implantable operation device according to aspect 21, wherein the pre-tensioning device comprises a flexible element.
23. The implantable operation device according to aspect 22, wherein the flexible element comprises at least one of a spring and an elastic material.
24. The implantable operation device according to any one of the preceding aspects, further comprising a gear system for transforming the mechanical force created by the electrical machine from a force having a first speed and a first strength to a force having a second lower speed and a second higher strength.
25. An implantable system comprising the implantable operation device according to any one of aspects 1-24, and a body engaging portion configured to exert a force on a body portion of the patient.
26. The implantable system according to aspect 25, wherein the body engaging portion comprises an implantable medical device for actively stretching a stomach wall of a patient for creating a sensation of satiety.
27. The implantable system according to aspect 25, wherein the body engaging portion comprises an implantable constriction device.
28. The implantable device according to aspect 27, wherein the implantable constriction device comprises an implantable constriction device for constricting a luminary organ of the patient.
29. The implantable device according to aspect 28, wherein the implantable constriction device comprises an implantable constriction device for constricting one of:
   an intestine of the patient,
   a blood vessel of the patient, and
   a vas deference of the patient.
30. The implantable system according to aspect 25, wherein the body engaging portion comprises an implantable element for actively emptying the urinary bladder of the patient.

### Aspect 396—Data_packet_encryption-Implant

1. A implantable medical device configured to receive remote instructions from an external system, the implantable medical device comprising:
   a wireless receiver configured to receive wirelessly transmitted data packets from the external system,
   a computing unit configured to:
      verify the electronic signature, and
      use a checksum provided in the data packet to verify the integrity of the instructions.
2. The implantable medical device according to aspect 1, wherein the computing unit is configured to decrypt the data packet.
3. The implantable medical device according to any one of aspects 1 and 2, wherein the computing unit is configured to use the checksum to verify that the bit stream making up the instructions is unchanged.
4. The implantable medical device according to any one of aspects 1 and 2, wherein the wireless receiver is part of a wireless transceiver.
5. The implantable medical device according to any one of aspects 1-4, wherein the computing unit comprises a memory unit configured to store electronic signatures, and wherein the computing unit is configured to verify the electronic signature my comparing the electronic signature with the electronic signatures stored in the memory unit.
6. The implantable medical device according to any one of the preceding aspects, wherein the implantable medical device comprises a control program configured to control at least one function of the implantable medical device, and wherein computing unit is configured to alter the control program on the basis of the received instructions.
7. The implantable medical device according to any one of the preceding aspects, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
8. The implantable medical device according to aspect 7, wherein the computing unit comprises a memory unit configured to store parameter values, and wherein the method further comprises the step of verifying that the instructions for altering the at least one parameter will result in the at least one parameter being updated to a parameter value comprised in the set of stored parameter values.
9. The implantable medical device according to any one of the preceding aspects, wherein the implantable medical device comprises a central unit, comprising at least one of a wireless receiver and a wireless transceiver, and a security module connected to the central unit, wherein the implantable medical device is configured to transfer the data packet from the central unit to the security module and wherein the security module is configured to performing at least a portion of at least one of the decryption and the signature verification.
10. The implantable medical device according to aspect 9, wherein the security module comprises a set of rules for accepting communication from the central unit, and wherein the security module is configured to verify compliance with the set of rules.
11. The implantable medical device according to aspect 10, wherein wireless receiver or wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.
12. The implantable medical device according to any one of the preceding aspects, wherein the implantable medical device is configured to at least one of decrypting the data packet and verifying the electronic signature using a private key of the implantable medical device.
13. The implantable medical device according to any one of aspects 10-12, wherein the private key is a non-extractable key.
14. The implantable medical device according to any one of aspects 10-13, wherein the implantable medical device is configured to perform a proof of possession operation comprising:
   transmitting, from the implantable medical device to the external system, a query based on a public key associated with the private key of the external system,
   receiving, at the implantable medical device, a response based on the possession of the private key in the external system, and
   verifying that the response based on the possession of the private key matches the query based on a public key.
15. The implantable medical device according to any one of the preceding aspects, wherein the implantable medical device is configured to communicate with the external system independently of time.
16. The implantable medical device according to any one of the preceding aspects, wherein the private key is provided in the implantable medical device by the manufacturer of the implantable medical device.
17. The implantable medical device according to aspect 16, wherein the private key is stored as hardware or software in the implantable medical device.
18. The implantable medical device according to any one of the preceding aspects 12-17, wherein the implantable medical device is configured to:
   verify a first electronic signature made using at least one of a first key and a second key, and
   verifying a second electronic signature made using at least one of a first key and a second key.
19. The implantable medical device according to aspect 18, wherein at least one of the first and second keys is a private key.
20. The implantable medical device according to aspect 18, wherein the first and second keys are different.
21. The implantable medical device according to aspect 20, wherein the first and second keys comprises at least one common element.
22. The implantable medical device according to any one of aspects 18-21, wherein the implantable medical device is configured to:
   verify a first electronic signature to allow communication from the external system to the implantable medical device, and
   verify a second electronic signature to allow an instruction received in the communication to alter the control program running on the implantable medical device.
23. The implantable medical device according to aspect 22, wherein the first electronic signature is an electronic signature linked to the user of the implantable medical device and the second electronic signature is an electronic signature linked to a healthcare provider.
24. The implantable medical device according to any one of aspects 12-23, wherein only a portion of the private key is needed to at least one of: decrypt the data packet and verify the electronic signature.
25. The implantable medical device according to any one of aspects 12-23, wherein the implantable medical device trusts any external device holding the private key.
26. The implantable medical device according to any one of the preceding aspects, wherein the implantable medical device is configured to receive the data packet comprising:
   at least one instruction signed by a private key of the external system, and
   a public key including information about which root have created the public key.
27. The implantable medical device according to any one of the preceding aspects, wherein the implantable medical device is configured to accept communication from an external system based on at least one password being provided to the implantable medical device.
28. The implantable medical device according to aspect 27, wherein the implantable medical device is configured to accept communication from an external system based on two passwords being provided to the implantable medical device.
29. The implantable medical device according to aspect 28, wherein the implantable medical device is configured to accept communication from an external system based on one patient password and one healthcare provider passwords being provided to the implantable medical device.
30. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a
first direction relative to the first member, for stretching the stomach wall between the first and second portion,
wherein at least one of the first and second member is flexible in a second direction substantially perpendicular to the first direction for at least one of: adapting the medical device to the curvature of the stomach of the patient, and facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and
   a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and a controller configured to:
      receive a signal from the sensor, and
      control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
47. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created, characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 447—Data_packet_encryption—External device
1. An external system for providing remote instructions to an implantable medical device, the external system being configured to:
   provide instructions to be transmitted to the implantable medical device,
   derive a checksum from the instructions,
   electronically sign the instructions and the checksum,
   form a data packet from the instructions, the electronic signature and the checksum, wherein the external system comprises a wireless transmitter configured to wirelessly send the data packet to the implantable medical device.
2. The external system according to aspect 1, wherein the external system is further configured to encrypt the data packet at the external system.
3. The external system according to any one of aspects 1 and 2, wherein the wireless transmitter is part of a wireless transceiver comprised in the external system.
4. The external system according to any one of the preceding aspects, wherein the external system comprises a first external device and a second external device, and wherein the first external device is configured to transmit the data packet to the second external device, and wherein the second external device is configured to transmit the data packet wirelessly to the implantable medical device without changing the data packet.
5. The external system according to any one of the preceding aspects, wherein the external system comprises a first external device and a second external device, and wherein the first external device is configured to transmit the data packet to the second external device, and wherein the second external device is configured to transmit the data packet wirelessly to the implantable medical device without full decryption of the data packet.
6. The external system according to any one of the preceding aspects, wherein the external system is configured to transmit at least one instruction for altering the control program of the implantable medical device, to the implantable medical device.
7. The external system according to any one of the preceding aspects, wherein the external system is configured to provide at least one instruction to the implantable medical device for altering at least one parameter for affecting the control of the implantable medical device.
8. The external system according to aspect 7, wherein the external system is configured to provide at least one instruction for updating at least one parameter of the control program to a parameter value comprised in a set of parameter values stored in the implantable medical device.
9. The external system according to any one of the preceding aspects, wherein the first external device is configured to send the data packet from the first external device to the second external device using a first network protocol and send the data packet from the second external device to the implantable medical device using a second network protocol.
10. The external system according to any one of the preceding aspects, wherein the first external device is configured to send the data packet from the first external device to the second external device using wired communication and send the data packet from the second external device to the implantable medical device using wireless communication.
11. The external system according to any one of aspects 1-9, wherein the first external device is configured to wirelessly send the data packet from the first external device to the second external device using a first network protocol, and wirelessly send the data packet from the second external device to the implantable medical device using a second network protocol.
12. The external system according to any one of aspects 1-9 or 11, wherein the first external device is configured to wirelessly send the data packet from the first external device to the second external device using a first frequency band, and wirelessly send the data packet from the second external device to the implantable medical device using a second frequency band.
13. The external system according to any one of the preceding aspects, wherein the first external device is configured to wirelessly send the data packet from the first external device to the second external device using a first wireless technology, and wirelessly send the data packet from the second external device to the implantable medical device using a second wireless technology.
14. The external system according to any one of the preceding aspects, wherein the external system is configured to electronically sign the instructions at the external system using a key of the external system.
15. The external system according to aspect 14, wherein the key is a non-extractable key.
16. The external system according to any one of aspects 14 and 15, wherein the second external device is configured to perform a proof of possession operation comprising the steps of:
   transmitting, form the first external device to the second external device, a query based on a public key associated with the private of the external system,
   receiving, at the second external device, a response based on the possession of the private key in the first external device, and
   verifying that the response based on the possession of the private key matches the query based on a public key.
17. The external system according to any one of the preceding aspects, wherein:
   the first external device is configured to form the data packet and electronically sign the instruction using a first private key, and
   the second external device is configured to:
      receive the data packet from the first external device,
      verify that the first external device is a trusted transmitter,
      in response to the verification, electronically sign the data packet using a second private key, and
   transmit the data packet from the second external device to the medical implant.
18. The external system according to any one of the preceding aspects, wherein the checksum is configured to verify that no changes have been made to the bit stream forming the instructions.
19. The external system according to any one of the preceding aspects, wherein the first external device is configured to at least one of: electronically sign the instructions and encrypt the data packet using a key placed on a key device external to the first external device.
20. The external system according to any one of the preceding aspects, wherein the external system further comprises a key device configured to hold at least one private key.
21. The external system according to aspect 20, wherein the key device comprises a wireless transmitter for wirelessly transmitting the at least one private key or a signal based on the private key, to the first external device.
22. The external system according to any one of the preceding aspects, wherein the second external device is configured to at least one of: electronically sign the instructions and encrypt the data packet using a key placed on a key device external to the second external device.
23. The external system according to any one of aspects 14-22, wherein the external system further comprises a second key device configured to hold at least one second private key.
24. The external system according to aspect 23, wherein the second key device comprises a wireless transmitter for wirelessly transmitting the at least one private key or a signal based on the private key to the second external device.
25. The external system according to aspect 14, further comprising a second key device comprising a wireless transmitter for wirelessly transmitting at least one second private key or a signal based on the second private key to the first external device.
26. The external system according to any one of aspects 14-25, wherein at least one of the key device and the second key device comprises at least one of: a key card, a wearable device and a handset.
27. The external system according to any one of the preceding aspects, wherein the first external device is configured to be unlocked by user credentials provided to the first external device.
28. The external system according to aspect 27, wherein the first external device is configured to be unlocked by user credentials comprising a username and a password.
29. The external system according to aspect 28, wherein the first external device is configured to be unlocked by user credentials comprising a PIN-code.
30. The external system according to any one of aspects 27-29, wherein the first external device is configured to verify the user credentials by comparing the user credentials with user credentials stored in the first external device.
31. The external system according to aspect 30, wherein the first external device is configured to verify the user credentials by comparing the user credentials with user credentials stored in the first external device by the manufacturer of the first external device.
32. The external system according to any one of aspects 27-31, wherein the first external device is configured verify the user credentials by comparing the user credentials with user credentials stored as hardware or software in the first external device.
33. The external system according to any one of aspects 27-32, wherein the first external device is configured verify the user credentials by communicating with a remote server.
34. The external system according to any one of the preceding aspects, wherein the second external device is configured to be unlocked by user credentials provided to the second external device.
35. The external system according to aspect 34, wherein the first external device is configured to be unlocked by user credentials comprising a username and a password.
36. The external system according to aspect 34, wherein the first external device is configured to be unlocked by user credentials comprising a PIN-code.
37. The external system according to aspect 36, wherein the second external device is configured to verify the user credentials by comparing the user credentials with user credentials stored in the second external device.
38. The external system according to aspect 37, wherein the second external device is configured to verify the user credentials by comparing the user credentials with user credentials stored in the second external device by the manufacturer of the second external device.
39. The external system according to any one of aspects 37 and 38, wherein the second external device is configured verify the user credentials by comparing the user credentials with user credentials stored as hardware or software in the second external device.
40. The external system according to any one of aspects 37-39, wherein the second external device is configured verify the user credentials by communicating with a remote server.
41. The external system according to any one of the preceding aspects, wherein the external system is configured to function without connection to the Internet.
42. The external system according to any one of the preceding aspects, wherein the external system is configured to communicate with the implantable medical device independently of time.
43. The external system according to any one of the preceding aspects 14-42, wherein the first and second private keys are different.
44. The external system according to aspect 43, wherein the first and second private keys comprises at least one common element.
45. The external system according to any one of aspects 14-44, wherein at least one first and second external device are configured to be unlocked by at least one of the first and second private key.
46. The external system according to any one of the preceding aspects, wherein the external system comprises a central server, and wherein the central server is configured to form a data packet from the instructions, the electronic signature and the checksum and further configured to provide the formed data packet to the first external device.
47. The external system according to aspect 46, wherein the central server can be accessed by at least one healthcare professional, such that the healthcare professional can provide input to the central server for forming the instructions to be sent to the implantable medical device.
48. The external system according to aspect 46, wherein the central server can be accessed by at least one patient, such that the patient can provide input to the central server for verifying at least one of: the authenticity of the healthcare professional and the correctness of the instructions.
49. The external system according to aspect 48, wherein the healthcare provider can electronically sign the instructions at the central server.
50. The external system according to any one of aspects 48 and 49, wherein the patient can electronically sign the instructions at the central server.
51. The external system according to any one of aspects 46-50, wherein the central server is configured to verify the authenticity of the first and second key and electronically sign the instructions using the first and second key.
52. The external system according to any one of the preceding aspects, wherein the second key is a user key, and wherein the external system is configured to use the second key for at least one of:
   approving that communication is transmitted to the implantable medical device, and
   approving that a healthcare provider prepares an instruction to the implantable medical device.
53. The external system according to aspect 52, wherein the approval step can be performed by first or second external device.
54. The external system according to any one of aspects 14-53, wherein the first key is required to create an instruction to the implantable medical device and the second key is required to transmit the created instruction to the implantable medical device.
55. The external system according to any one of aspects 2-54, wherein at least one of the first and second external device comprises an input button configured to be used for verifying user presence.
56. The external system according to aspect 55, wherein the input button con be configured to replace at least one of:
   input of at least one key to at least one of the first and second external device, and
   input of credentials into at least one of the first and second external device.
56. The external system according to aspect 55, wherein the input button is configured to replace the second key.
57. The external system according to any one of the preceding aspects, wherein the external system is configured to transmit the data packet to the implantable medical device, and wherein the data packet comprises:
   at least one instruction signed by a first key and
   a public key including information about which root have created the public key.
58. The external system according to any one of aspects 2-57, wherein at least one of the first and second external device is configured to enable communication with the implantable medical device based on at least one password being provided to at least one of the first and second external device.
59. The external system according to aspect 58, wherein at least one of the first and second external device is configured to enable communication with the implantable medical device based on two passwords being provided to at least one of the first and second external device.
60. The external system according to aspect 59, wherein at least one of the first and second external device is configured to enable communication with the implantable medical device based on one patient password and one healthcare provider passwords being provided to at least one of the first and second external device.
61. The external system according to any one of the preceding aspects, wherein at least one of the first and second external devices are configured to perform a verification query operation with at least one of the first and second key device, the verification query operation comprising:
   transmitting, from the first or second external devices, a query comprising a computational challenge to at least one of the first and second key device,
   receiving, at the first or second external devices, a response based on the transmitted computational challenge, and
   verifying, at the first or second external devices, the received response.
62. The external system according to aspect 61, wherein at least one of the first and second external devices are configured to perform a verification query operation in the form of a proof of possession operation comprising:
   receiving a public key of at least one of the first and second key devices, the public key being associated with a private key of the first or second key device,
   transmitting, from at least one of the first and second external devices, a computational challenge to the first or second key device, based on the public key received from the first or second key device,
   receiving a response from the first or second key device based on the possession of the private key in the first or second key device, and
   verifying that the response based on the possession of the private key matches the query based on a public key.
63. A medical system comprising the external system according to any one of the preceding aspects and an implantable medical device.
64. The medical system according to aspect 63, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
65. The medical system according to aspect 63, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
66. The medical system according to aspect 63, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
67. The medical system according to aspect 63, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
68. The medical system according to aspect 63, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second
   portion, an acoustic sensor configured to sense at least one sound related to the patient swallowing,
   and
      a controller configured to:
      receive a signal from the acoustic sensor, and control the operation device on the basis of the signal received from the acoustic sensor.
69. The medical system according to aspect 63, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
70. The medical system according to aspect 63, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
71. The medical system according to aspect 63, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
72. The medical system according to aspect 63, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
73. The medical system according to aspect 63, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
74. The medical system according to aspect 63, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
75. The medical system according to aspect 63, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
76. The medical system according to aspect 63, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
77. The medical system according to aspect 63, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall, an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device: from operating the first member to move in the first direction to operating
   the first member to move in the second direction, or from operating the first member to move in the first direction to operating the third member to move in the third direction.
78. The medical system according to aspect 63, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
79. The medical system according to aspect 63, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
80. The medical system according to aspect 63, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
      the stretching device is adapted to stretch a portion of the stomach wall between the first area and the second area.
81. The medical system according to aspect 63, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created, characterized in that the operable stretching device is adapted to be placed against the outside of the stomach wall.
82. The medical system according to aspect 63, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
83. The medical system according to aspect 63, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with

### Aspect 447B—Data_packet_encryption-Method

1. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions.
2. The method according to aspect 1, further comprising the steps of encrypting the data packet at the external system using a private key of the external system, and decrypting, at the implantable medical device, the data packet using a private key of the implantable medical device.
3. The method according to any one of aspects 1 and 2, wherein the step of verifying the electronic signature comprises comparing the electronic signature with electronic signatures stored in the implantable medical device.
4. The method according to any one of aspects 1-3, wherein the step of wirelessly sending the data packet to the implantable medical device comprises sending the data packet from a first external device to a second external device using wired communication and wirelessly sending the data packet from the second external device to the implantable medical device.
5. The method according to any one of aspects 1-4, wherein the step of wirelessly sending the data packet to the implantable medical device comprises sending the data packet from a first external device to a second external device and further wirelessly sending the data packet from the second external device to the implantable medical device, and wherein the second external device transmits the data packet without changing the data packet.
6. The method according to any one of aspects 1-5, wherein the step of wirelessly sending the data packet to the implantable medical device comprises sending the data packet from a first external device to a second external device and further wirelessly sending the data packet from the second external device to the implantable medical device, and wherein the second external device transmits the data packet without full decryption.
7. The method according to any one of the preceding aspects, wherein the implantable medical device comprises a control program configured to control at least one function of the implantable medical device, and wherein the method further comprises altering the control program on the basis of the received instructions.
8. The method according to any one of the preceding aspects, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
9. The method according to aspect 8, wherein the implantable medical device comprises a set of stored parameter values, and wherein the method further comprises the step of verifying that the instructions for altering the at least one parameter will result in the at least one parameter being updated to a parameter value comprised in the set of stored parameter values.
10. The method according to any one of the preceding aspects, wherein the step of wirelessly sending the data packet to the implantable medical device comprises:
   wirelessly sending the data packet from a first external device to a second external device using a first network protocol, and
   wirelessly sending the data packet from the second external device to the implantable medical device using a second network protocol.
11. The method according to any one of the preceding aspects, wherein the step of wirelessly sending the data packet to the implantable medical device comprises:
   wirelessly sending the data packet from a first external device to a second external device using a first frequency band, and
   wirelessly sending the data packet from the second external device to the implantable medical device using a second frequency band.
12. The method according to any one of the preceding aspects, wherein the step of wirelessly sending the data packet to the implantable medical device comprises:
   wirelessly sending the data packet from a first external device to a second external device using a first wireless technology, and
   wirelessly sending the data packet from the second external device to the implantable medical device using a second wireless technology, wherein the first wireless technology has an effective range being one of: 2 times, 4 times, 8 times 20 times, 50 times or 100 times longer than the first wireless technology.
13. The method according to any one of the preceding aspects, wherein the implantable medical device comprises a central unit, comprising a wireless transceiver, and a security module connected to the central unit, wherein the step of decrypting, at the implantable medical device, the data packet, comprises transferring the data packet from the central unit to the security module, and performing at least a portion of the decryption in the security module.
14. The method according to aspect 13, wherein the security module comprises a set of rules for accepting communication from the central unit, and wherein the step of transferring the data packet from the receiving unit of the implant to the security module comprises verifying compliance with the set of rules.
15. The method according to aspect 14, wherein wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.
16. The method according to any one of the preceding aspects, wherein the step of electronically signing the instructions at the external system comprises electronically signing the instructions at the external system using a private key of the external system.
17. The method according to aspect 16, wherein the private key is a non-extractable key.
18. The method according to any one of aspects 16 and 17, wherein the step of verifying the electronic signature comprises performing a proof of possession operation comprising the steps of:
   transmitting, form the medical device to the external system, a query based on a public key associated with the private of the external system,
   receiving, at the medical device, a response based on the possession of the private key in the external system, and
   verifying that the response based on the possession of the private key matches the query based on a public key.
19. The method according to any one of the preceding aspects, wherein the step of forming the data packet is performed at a first external device, and wherein the step of electronically signing the instructions comprises electronically signing the instruction using a first private key, and wherein the method further comprises:
   transmitting the data packet from the first external device to a second external device,
   verifying, at the second external device, that the transmitter is a trusted transmitter,
   in response to the verification, electronically signing the data packet using a second private key, and
   transmitting the data packet from the second external device to the medical implant,
   and verifying, at the medical implant, the electronic signatures generated using the first and second private keys.
   using the checksum to verify the integrity of the instructions.
20. The method according to any one of the preceding aspects, wherein the step of electronically signing the instructions and the checksum, at the external system, comprising signing the instructions and the checksum with the use of a key placed on a key device separate from at least one of the first and second external device.
21. The method according to any one of the preceding aspects, wherein the step of electronically signing the instructions and the checksum, at the external system, comprising signing the instructions and the checksum with the use of a key placed on a key device comprising a wireless transmitter for wirelessly transmitting the at least one private key to at least one of the first and second external device.
22. The method according to any one of aspects 20 and 21, wherein the step of electronically signing the instructions and the checksum, at the external system, further comprises signing the instructions and the checksum with the use of a second key placed on the key device or on a second key device, separate from at least one of the first and second external device.
23. The method according to any one of aspects 21 and 22, wherein at least one of the key device and the second key device comprises at least one of: a key card, a wearable device and a handset.
24. The method according to any one of the preceding aspects, further comprising the step of unlocking at least one of the first and second external device using user credentials provided to the first and/or second external device.
25. The method according to aspect 24, wherein the step of unlocking at least one of the first and second external devices comprises unlocking at least one of the first and second external devices using a username and a password.
26. The method according to aspect 24, wherein the step of unlocking at least one of the first and second external devices comprises unlocking at least one of the first and second external devices using a PIN-code.
27. The method according to aspect 24, wherein the step of unlocking at least one of the first and second external devices comprises verifying, at the at least one first or second external devices, the user credentials by comparing the user credentials with user credentials stored in at least one of the first and second external devices.
28. The method according to aspect 24, wherein the step of unlocking at least one of the first and second external devices comprises verifying, at the at least one first or second external devices, the user credentials by comparing the user credentials with user credentials stored in at least one of the first and second external devices by the manufacturer of at least one of the first and second external devices.
29. The method according to any one of aspects 24-28, wherein the step of unlocking at least one of the first and second external devices comprises verifying, at the at least one first or second external devices, the user credentials by comparing the user credentials with user credentials stored as hardware or software in at least one of the first and second external devices.
30. The method according to any one of aspects 24-29, wherein the step of unlocking at least one of the first and second external devices comprises verifying, at the at least one first or second external devices, the user credentials by communicating with a remote server.
31. The method according to any one of the preceding aspects, wherein the method is performed without connection to the Internet.
32. The method according to any one of the preceding aspects, wherein the method is configured to be performed independently of time.
33. The method according to any one of the preceding aspects, wherein the first and second keys are different.
34. The method according to any one of the preceding aspects, wherein at least one of the first and second keys are private.
35. The method according to aspect 34, wherein the first and second private keys comprises at least one common element.
36. The method according to any one of aspects 4-35, comprising unlocking at least one of the first and second external devices using at least one of the first and second private key.
37. The method according to any one of the preceding aspects, wherein the step of electronically signing the instructions and the checksum is performed at a central server of the external system.
38. The method according to aspect 37, further comprising: the central server being accessed by at least one healthcare professional, and the healthcare professional providing input to the central server for forming the instructions to be sent to the implantable medical device.
39. The method according to aspect 37, further comprising the central server being accessed by at least one patient, such that the patient can provide input to the central server for verifying at least one of: the authenticity of the healthcare professional and the correctness of the instructions.
40. The method according to aspect 38, further comprising the healthcare electronically signing the instructions at the central server.
41. The method according to aspect 38, further comprising the patient electronically signing the instructions at the central server.
42. The method according to any one of aspects 37-41, further comprising the steps of:
   verifying the authenticity of the first and second key at the central server, and
   electronically sign the instructions using the first and second key.
42. The method according to any one of the preceding aspects, wherein the second key is a user key, and wherein the method comprises the steps of using the second key for at least one of:
   approving that communication is transmitted to the implantable medical device, and
   approving that a healthcare provider prepares an instruction to the implantable medical device.
43. The method according to aspect 42, wherein the approval step can be performed by first or second external device.
44. The method according to any one of aspects 4-43, wherein the first key is required to create an instruction to the implantable medical device and the second key is required to transmit the created instruction to the implantable medical device.
45. The method according to any one of aspects 4-44, wherein at least one of the first and second external device comprises an input button, and wherein the method further comprises the step of pressing the button for verifying user presence.
46. The method according to aspect 45, wherein the input button is placed on the second external device.
47. The method according to any one of the preceding aspects, wherein the trental of the data packet comprises transmittal of:
   at least one instruction signed by a first key, and
   a public key including information about which root have created the public key.
48. The method according to any one of aspects 4-47, further comprising enabling communication between the implantable medical device and at least one of the first and second medical device based on at least one password being provided to at least one of the first and second external device.
49. The method according to any one of aspects 4-47, further comprising enabling communication between the implantable medical device and at least one of the first and second medical device based on two passwords being provided to at least one of the first and second external device.
50. The method according to aspect 49, wherein the first password is a patient password and the second password is a healthcare provider passwords.
51. The method according to any one of the preceding aspects, further comprising at least one of the first and second external devices performing a verification query operation with at least one of the first and second key devices, the verification query operation comprising:
   transmitting, from the first or second external devices, a query comprising a computational challenge to at least one of the first and second key device,
   receiving, at the first or second external devices, a response based on the transmitted computational challenge, and
   verifying, at the first or second external devices, the received response.
52. The method according to aspect 51, wherein the verification query operation is in the form of a proof of possession operation comprising:
   receiving a public key of at least one of the first and second key devices, the public key being associated with a private key of the first or second key device,
   transmitting, from at least one of the first and second external devices, a computational challenge to the first or second key device, based on the public key received from the first or second key device,
   receiving a response from the first or second key device based on the possession of the private key in the first or second key device, and
   verifying that the response based on the possession of the private key matches the query based on a public key.

### Aspect 447C—Single-use_codes_encryption

1. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes.
2. The method according to aspect 1, further comprising the steps of:
   wirelessly sending position information from the external device to the implantable medical device, and
   using the information at the implantable medical device for selecting the code from the list of codes.
3. The method according to aspect 1, wherein the step of encrypting, at the external system, the instructions using a code from a position on the list of codes comprises selecting the code on a current position on the list of codes, wherein the method further comprises the step of updating the current position to a new current position after using the code.
4. The method according to any one of aspects 1 and 3, wherein the step of decrypting, at the implantable medical device, the instructions using a code from a position on the list of codes comprises selecting the code on a current position on the list of codes, wherein the method further comprises the step of updating the current position to a new current position after using the code.
5. The method according to any one of aspects 3 and 4, wherein the current position comprises a number and wherein the step of updating the current position comprises updating the number to a sequential number.
6. The method according to any one of aspects 1-5, wherein the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without changing the encrypted instructions.
7. The method according to any one of aspects 1-6, wherein the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without full decryption.
8. The method according to any one of the preceding aspects, wherein the implantable medical device comprises a control program configured to control at least one function of the implantable medical device, and wherein the method further comprises altering the control program on the basis of the received instructions.
9. The method according to any one of the preceding aspects, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
10. The method according to aspect 9, wherein the implantable medical device comprises a set of stored parameter values, and wherein the method further comprises the step of verifying that the instructions for altering the at least one parameter will result in the at least one parameter being updated to a parameter value comprised in the set of stored parameter values.
11. The method according to any one of the preceding aspects, wherein the step of wirelessly sending the encrypted instructions to the implantable medical device comprises:
   wirelessly sending the encrypted instructions from a first external device to a second external device using a first network protocol, and
   wirelessly sending the encrypted instructions from the second external device to the implantable medical device using a second network protocol.
11. The method according to any one of the preceding aspects, wherein the step of wirelessly sending the encrypted instructions to the implantable medical device comprises:
   wirelessly sending the encrypted instructions from a first external device to a second external device using a first frequency band, and
   wirelessly sending the encrypted instructions from the second external device to the implantable medical device using a second frequency band.
12. The method according to any one of the preceding aspects, wherein the step of wirelessly sending the encrypted instructions to the implantable medical device comprises:
   wirelessly sending the encrypted instructions from a first external device to a second external device using a first wireless technology, and
   wirelessly sending the encrypted instructions from the second external device to the implantable medical device using a second wireless technology, wherein the first wireless technology has an effective range being one of: 2 times, 4 times, 8 times 20 times, 50 times or 100 times longer than the first wireless technology.
13. The method according to any one of the preceding aspects, wherein the implantable medical device comprises a central unit, comprising a wireless transceiver, and a security module connected to the central unit, wherein the step of decrypting, at the implantable medical device, the encrypted instructions, comprises transferring the encrypted instructions from the central unit to the security module, and performing at least a portion of the decryption in the security module.
14. The method according to aspect 13, wherein the security module comprises a set of rules for accepting communication from the central unit, and wherein the step of transferring the encrypted instructions from the receiving unit of the implant to the security module comprises verifying compliance with the set of rules.
15. The method according to aspect 14, wherein wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.
16. The method according to any one of the preceding aspects, wherein the step of electronically signing the instructions at the external system comprises electronically signing the instructions at the external system using a private key of the external system.
17. The method according to aspect 16, wherein the private key is a non-extractable key.
18. The method according to any one of the preceding aspects, wherein the step of wirelessly sending the encrypted instructions to the implantable medical device comprises:
   wirelessly sending the encrypted instructions from a first external device to a second external device using a first wireless technology, and
   wirelessly sending the encrypted instructions from the second external device to the implantable medical device using a second wireless technology, wherein the first wireless technology has an effective range being one of: 2 times, 4 times, 8 times 20 times, 50 times or 100 times longer than the first wireless technology.
      Aspect 384SE—eHealth_General_Communication_Dual
1. An external device configured for communication with an implantable medical device
when implanted in a patient, the external device comprising:
at least one first wireless transceiver configured for communication with the implantable medical device using a first network protocol, for determining a distance between the external device and the implantable medical device, and
at least one second wireless transceiver configured for communication with the implantable medical device using a second network protocol, for transferring data between the external device and the implantable medical device.
2. The external device according to aspect 1, wherein the first wireless transceiver comprises an UWB transceiver.
3. The external device according to aspect 1, wherein the first wireless transceiver is configured for transcutaneous energy transfer for at least one of: powering an energy consuming component of the implantable medical device, and charging an implantable energy storage unit.
4. The external device according to any one of the preceding aspects, wherein the second network protocol is a standard network protocol.
5. The external device according to any one of the preceding aspects, wherein the second wireless transceiver comprises a Bluetooth transceiver.
6. The external device according to any one of the preceding aspects, wherein the external device is further configured to communicate with a second external device using said at least one wireless transceiver.
7. The external device according to any one of the preceding aspects, wherein the external device is configured for determining a distance between the external device and the implantable medical device by determining the RSSI.
8. The external device according to any one of aspects 4-7, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
9. The external device according to any one of the preceding aspects, wherein a communication range of the first network protocol is less than a communication range of the second network protocol.
10. The external device according to any one of the preceding aspects, wherein a frequency band of the first network protocol differs from a frequency band of the second network protocol.
11. The external device according to any one of the preceding aspects, wherein the external device is configured to authenticate the implantable medical device if the determined distance between the external device and the implantable medical device is less than a predetermined threshold value.
12. The external device according to aspect 11, wherein the external device is configured to allow the transfer of data between the external device and the implantable medical device after the implantable medical device has been authenticated.
13. The external device according to any one of the preceding aspects, wherein the external device is one from the list of:
   a wearable external device, and
   a handset.
14. A medical system comprising the external system according to any one of the preceding aspects and an implantable medical device.
15. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
16. The medical system according to aspect 14, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
a second member comprising a second stomach engager portion configured to be fixated to a second portion of the stomach wall,
a hydraulic operation device for operating the second member to displace the second member in a first direction relative to the first member, for stretching the stomach wall between the first and second portions, and
a main portion, wherein:
   the first and second members are connected to the main portion, and wherein at least the second member is configured to bend or pivot in relation to the main portion, such that the second member can be displaced in the first direction relative to the first member by the second member bending or pivoting in relation to the main portion,
   at least the second member comprises a fluid chamber at least partially enclosed by a flexible wall portion, and
   the flexible wall portion causes the second member to bend or pivot when hydraulic fluid is moved to or from the fluid chamber for stretching the stomach wall between the first and second portions.
17. The medical system according to aspect 14, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
a second member comprising a second stomach engager portion configured to be fixated to a second portion of the stomach wall,
an operation device for operating the second member to displace the second member in a first direction, for stretching the stomach wall between the first and second portions, and
a main portion, wherein:
   the first and second members are connected to the main portion,
   at least the second member is configured to bend or pivot in relation to the main portion, such that the second member can be displaced in a direction away from the first member by the second member bending or pivoting in relation to the main portion,
   the operation device comprises a first flexible cable for pulling on the second member for displacing the second member in the direction away from the first member, and
   the first flexible cable is fixated to the second member at the distal half of thereof.
18. The medical system according to aspect 14, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
19. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
20. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
21. The medical system according to aspect 14, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
22. The medical system according to aspect 14, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
23. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
24. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
25. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
26. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
27. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
28. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
29. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
30. The medical system according to aspect 14, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. The medical system according to aspect 14, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein the stretching device is adapted to stretch a portion of the stomach wall between the first area and the second area.
32. The medical system according to aspect 14, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
33. The medical system according to aspect 14, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. The medical system according to aspect 14, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
35. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device.
36. The implantable medical device according to aspect 35, wherein the first wireless transceiver comprises an UWB transceiver.
37. The implantable medical device according to aspect 35, wherein the first wireless transceiver is configured for transcutaneous energy transfer for at least one of: powering an energy consuming component of the implantable medical device, and charging an implantable energy storage unit.
38. The implantable medical device according to any one of aspects 35-37, wherein the second network protocol is a standard network protocol.
39. The implantable medical device according to any one of aspects 35-38, wherein the second wireless transceiver comprises a Bluetooth transceiver.
40. The implantable medical device according to any one of aspects 35-39, wherein the implantable medical device is further configured to communicate with a second external device using said at least one wireless transceiver.
41. The implantable medical device according to any one of aspects 35-40, wherein the implantable medical device is configured for determining a distance between the external device and the implantable medical device by determining the RSSI.
42. The implantable medical device according to any one of aspects 35-41, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
43. The implantable medical device according to any one of the aspects 35-42, wherein a communication range of the first network protocol is less than a communication range of the second network protocol.
44. The implantable medical device according to any one of aspects 35-43, wherein a frequency band of the first network protocol differs from a frequency band of the second network protocol.
45. The implantable medical device according to any one of aspects 35-44, wherein the implantable medical device is configured to authenticate the external device if the determined distance between the external device and the implantable medical device is less than a predetermined threshold value.
46. The implantable medical device according to aspect 45, wherein the implantable medical device is configured to allow the transfer of data between the implantable medical device and the external device after the external device has been authenticated.
47. The implantable medical device according to aspects 35-46, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of: adapting the medical device to the curvature of the stomach of the patient, and facilitating insertion of the medical device into the body of the patient.
48. The implantable medical device according to any one of aspects 35-46, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
49. The implantable medical device according to any one of aspects 35-46, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
a second member comprising a second stomach engager portion configured to be fixated to a second portion of the stomach wall,
a hydraulic operation device for operating the second member to displace the second member in a first direction relative to the first member, for stretching the stomach wall between the first and second portions, and
a main portion, wherein:
   the first and second members are connected to the main portion, and wherein at least the second member is configured to bend or pivot in relation to the main portion, such that the second member can be displaced in the first direction relative to the first member by the second member bending or pivoting in relation to the main portion,
   at least the second member comprises a fluid chamber at least partially enclosed by a flexible wall portion, and
   the flexible wall portion causes the second member to bend or pivot when hydraulic fluid is moved to or from the fluid chamber for stretching the stomach wall between the first and second portions.
50. The implantable medical device according to any one of aspects 35-46, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
51. The implantable medical device according to any one of aspects 35-46, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and
   a force transferring element configured to transfer force hydraulically or mechanically from the remote unit to the main portion, for operating the second member.
52. The implantable medical device according to any one of aspects 35-46, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
53. The implantable medical device according to any one of aspects 35-46, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and a controller configured to:
      receive a signal from the sensor, and
      control the operation device on the basis of the signal received from the sensor.
54. The implantable medical device according to any one of aspects 35-46, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
55. The implantable medical device according to any one of aspects 35-46, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
56. The implantable medical device according to any one of aspects 35-46, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
57. The implantable medical device according to any one of aspects 35-46, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced: relative to the second member, and within the footprint of the second member, for stretching a portion of the stomach wall of the patient between the second and first member.
58. The implantable medical device according to any one of aspects 35-46, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
59. The implantable medical device according to any one of aspects 35-46, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
60. The implantable medical device according to any one of aspects 35-46, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
61. The implantable medical device according to any one of aspects 35-46, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
62. The implantable medical device according to any one of aspects 35-46, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
63. The implantable medical device according to any one of aspects 35-46, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
64. The implantable medical device according to any one of aspects 35-46, wherein the implantable medical device comprises a device for treating obesity of a patient comprising:
   at least one operable stretching device (10; 110) implantable in the patient and adapted to stretch a portion of the patient's stomach wall (12), and
an implantable control unit (42) for automatically controlling the operable stretching device, when the control unit and stretching device are implanted, to stretch the stomach wall portion in connection with the patient eating such that satiety is created,
characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
65. The implantable medical device according to any one of aspects 35-46, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
66. The implantable medical device according to any one of aspects 35-46, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
67. The implantable medical device according to any one of aspects 35-46, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 385SE—eHealth_General_General_Remote Display Portal
1. An external device configured for communication with an implantable medical device
when implanted in a patient, the patient external device comprising: a wireless communication unit configured for wireless transmission of control commands to the implantable medical device and configured for wireless communication with a patient display device, and
a computing unit configured for running a control software for creating the control commands for the operation of the implantable medical device, wherein the computing unit is configured to: transmit a control interface as a remote display portal to a patient display device configured to display the control interface to a user, receive user input from the patient display device, and transform the user input into the control commands for wireless transmission to the implantable medical device.
2. The external device according to aspect 1, wherein the wireless communication unit comprises a wireless transceiver for: wireless transmission of control commands to the implantable medical device, and wireless transmission of the control interface as the remote display portal to the patient display device.
3. The external device according to aspect 1, wherein the wireless communication unit comprises:
a first wireless transceiver for wireless transmission of control commands to the implantable medical device, and
a second wireless transceiver for wireless transmission of the control interface to the patient display device.
4. The external device according to any one of the preceding aspects, wherein the wireless communication unit is configured for wireless communication with the patient display device using a standard network protocol.
5. The external device according to any one of the preceding aspects, wherein the wireless communication unit is configured for wireless communication with the implantable medical device using a proprietary network protocol.
6. The external device according to any one of the preceding aspects, wherein the wireless communication unit comprises a Bluetooth transceiver.
7. The external device according to aspect 6, wherein at least one of the first and second wireless transceiver comprises a Bluetooth transceiver.
8. The external device according to any one of aspects 1-5, wherein the wireless communication unit comprises a UWB transceiver.
9. The external device according to aspect 8, wherein at least one of the first and second wireless transceiver comprises a UWB transceiver.
10. The external device according to aspect 1, wherein the wireless communication unit comprises:
at least one first wireless transceiver configured for communication with the implantable medical device using a first network protocol, for determining a distance between the patient external device and the implantable medical device, and at least one second wireless transceiver configured for communication with the implantable medical device using a second network protocol, for transferring data between the patient external device and the implantable medical device.
11. The external device according to aspect 3, wherein the first wireless transceiver is configured for transcutaneous energy transfer for at least one of: powering an energy consuming component of the implantable medical device, and charging an implantable energy storage unit.
12. The external device according to aspect 4, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
13. The external device according to any one of aspects 3-12, wherein a communication range of the first wireless transceiver is less than a communication range of the second wireless transceiver.
14. A medical system comprising the external system according to any one of the preceding aspects and an implantable medical device.
15. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
16. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
17. The medical system according to aspect 14, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
a second member comprising a second stomach engager portion configured to be fixated to a second portion of the stomach wall,
an operation device for operating the second member to displace the second member in a first direction, for stretching the stomach wall between the first and second portions, and
a main portion, wherein:
   the first and second members are connected to the main portion,
   at least the second member is configured to bend or pivot in relation to the main portion, such that the second member can be displaced in a direction away from the first member by the second member bending or pivoting in relation to the main portion,
   the operation device comprises a first flexible cable for pulling on the second member for displacing the second member in the direction away from the first member, and
   the first flexible cable is fixated to the second member at the distal half of thereof.
18. The medical system according to aspect 14, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
19. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
20. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
21. The medical system according to aspect 14, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
22. The medical system according to aspect 14, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
23. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
24. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
25. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
26. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
27. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
28. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
29. The medical system according to aspect 14, wherein the implantable medical device comprises an implantable operation device for operating a body engaging portion of a medical implant, the implantable operation device comprising:
   an electrical machine for transforming electrical energy to mechanical force, a transmission for transforming rotational force created by the electrical machine to a
   linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
30. The medical system according to aspect 14, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. The medical system according to aspect 14, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
32. The medical system according to aspect 14, wherein the implantable medical device comprises an obesity treatment device comprising:
at least one operable stretching device (10; 10″; 110a; 110b; 410b; 50; 810) implantable in a patient and adapted to stretch a portion of the patient's stomach wall (12) and
an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
33. The medical system according to aspect 14, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. The medical system according to aspect 14, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 386SE—eHealth_General_General_Appln_App
1. A patient display device for communication with a patient external device for communication with an implantable medical device, when implanted, the patient display device comprising:
a wireless communication unit,
a display, and
an input device for receiving implant control input from the user, wherein the patient display device is configured to:
run a first application for wireless communication with a server and/or DDI, and
run a second application for wireless communication with the patient external device for transmission of the implant control input to a remote display portal of the patient external device for the communication with the implantable medical device, wherein the second application is configured to be accessed through the first application.
2. The patient display device according to aspect 1, wherein the first log-in is a PIN-based log-in.
3. The patient display device according to aspect 1, wherein at least one of the first and second log-in is a log-in based on a biometric input or a hardware key.
4. The patient display device according to any one of aspects 1-3, wherein the patient display device further comprises an auxiliary wireless communication unit, and wherein the auxiliary wireless communication unit is configured to be disabled to enable wireless communication with the patient external device.
5. The patient display device according to any one of aspects 1-4, wherein the patient display device is configured to wirelessly receive an implant control interface as a remote display portal from the patient external device to be displayed on the display.
6. The patient display device according to any one of aspects 1-5, wherein the wireless communication unit is configured for wireless communication with the patient external device using a standard network protocol.
7. The patient display device according to any one of aspects 1-5, wherein the wireless communication unit is configured for wireless communication with the patient external device using a proprietary network protocol.
8. The patient display device according to any one of aspects 1-5, wherein the wireless communication unit is configured for wireless communication with the patient external device using a first network protocol and with the server using a second network protocol.
9. The patient display device according to any one of aspects 1-5, wherein the wireless communication unit is configured for wireless communication with the patient external device using a first frequency band and with the server using a second frequency band.
10. The patient display device according to any one of aspects 1-9, wherein the wireless communication unit comprises a Bluetooth transceiver.
11. The patient display device according to any one of aspects 1-10, wherein the wireless communication unit comprises a UWB transceiver.
12. The patient display device according to aspect 6, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
13. The patient display device according to aspect 4, wherein a communication range of the wireless communication unit is less than a communication range of the auxiliary wireless communication unit.
14. A medical system comprising the patient display device according to any one of the preceding aspects and an implantable medical device.
15. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
16. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
17. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
18. The medical system according to aspect 14, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
19. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
20. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
21. The medical system according to aspect 14, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
22. The medical system according to aspect 14, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
23. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
24. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
25. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
26. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
27. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
28. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
29. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
30. The medical system according to aspect 14, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. The medical system according to aspect 14, wherein the implantable medical device comprises a device for treating obesity of a patient, the device comprising:
at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and the second part is adapted to be engaged to a second area of the stomach wall, and wherein the stretching device is adapted to stretch a portion of the stomach wall between the first area and the second area.
32. The medical system according to aspect 14, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created, characterized in that the operable stretching device is adapted to be placed against the outside of the stomach wall.
33. The medical system according to aspect 14, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. The medical system according to aspect 14, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 387SE—eHealth_General_Encryption_End-to-End
1. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption.
2. The communication system according to aspect 1, wherein the patient display device is configured to wirelessly receive an implant control interface from the patient external device to be displayed on the display.
3. The communication system according to any one of aspects 1-2, wherein at least two of:
the wireless communication unit of the server,
the wireless communication unit of the patient display device,
the wireless communication unit of the patient external device, and
the wireless communication unit of the implantable medical device, is configured for wireless communication using a standard network protocol.
4. The communication system according to any one of aspects 1-2, wherein at least two of:
the wireless communication unit of the server,
the wireless communication unit of the patient display device,
the wireless communication unit of the patient external device, and
the wireless communication unit of the implantable medical device, is configured for wireless communication using a proprietary network protocol.
5. The communication system according to any one of aspects 1-4, wherein the wireless communication unit of the patient external device is configured to: use a first network protocol for communication with the implantable medical device and use a second network protocol for communication with the server, or use a first network protocol for communication with the implantable medical device and use a second network protocol for communication with the patient display device.
6. The communication system according to any one of aspects 1-5, wherein the wireless communication unit of the patient external device is configured to: use a first frequency band for communication with the implantable medical device and use a second frequency band for communication with the server, or use a first frequency band for communication with the implantable medical device and use a second frequency band for communication with the patient display device.
7. The communication system according to any one of aspects 1-6, wherein the wireless communication unit of the patient display device is configured to use a first network protocol for communication with the patient external device and use a second network protocol for communication with the server.
8. The communication system according to any one of aspects 1-7, wherein the wireless communication unit of the patient display device is configured to use a first frequency band for communication with the patient external device and use a second frequency band for communication with the server.
9. The communication system according to any one of aspects 1-8, wherein the wireless communication unit of the server is configured to use a first network protocol for communication with the patient external device and use a second network protocol for communication with the patient display device.
10. The communication system according to any one of aspects 1-9, wherein the wireless communication unit of the server is configured to use a first frequency band for communication with the patient external device and use a second frequency band for communication with the patient display device.
11. The communication system according to any one of aspects 1-10, wherein the wireless communication unit of at least one of the server, the patient display device, the patient external device, and the implantable medical device comprises a Bluetooth transceiver.
12. The communication system according to any one of aspects 1-11, wherein the wireless communication unit of at least one of the server, the patient display device, the patient external device, and the implantable medical device comprises a UWB transceiver.
13. The communication system according to aspect 3, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
14. A medical system comprising the communication system according to any one of the preceding aspects and an implantable medical device.
15. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
16. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
17. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
18. The medical system according to aspect 14, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
19. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
20. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
21. The medical system according to aspect 14, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
22. The medical system according to aspect 14, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
23. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
24. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
25. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
26. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
27. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
28. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
29. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
30. The medical system according to aspect 14, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. The medical system according to aspect 14, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
32. The medical system according to aspect 14, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
33. The medical system according to aspect 14, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. The medical system according to aspect 14, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 396SE—eHealth_General_Communication_Dual
1. A system configured for changing pre-programmed treatment settings of an implantable medical device, when implanted in a patient, from a distant remote location in relation to the patient, the system comprising:
at least one health care provider, HCP, EID external device, and a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of: a reading slot or comparable for the HCP private key device, a RFID communication, and
other close distance wireless activation communication or electrical direct contact; wherein said HCP EID external device further comprises at least one wireless transceiver configured for communication with a data infrastructure server, DDI, through a first network protocol, wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function.
2. The system configured for changing pre-programmed treatment settings of an implantable medical device according to aspect 1, wherein the patient providing a patient private key device adapted to be provided to the patient EID external device by the patient via at least one of: a reading slot or comparable for the patient private key device, a RFID communication or other close distance wireless activation communication or electrical direct contact,
wherein said patient EID external device comprises at least one of: a reading slot or comparable for the HCP private key device, a RFID communication, and
other close distance wireless activation communication or electrical direct contact.

3. The system configured for changing pre-programmed treatment settings of an implantable medical device according to aspect 1-2, wherein the wherein said patient EID external device further comprises at least one wireless transceiver configured for communication with the implanted medical device through a second network protocol.
4. The system according to aspect 1-3, wherein at least one of the patient private key device or HCP private key device comprises a hardware key.
5. The system according to any preceding aspect, wherein the private key device is at least one of, a smartcard, a key-ring device, a watch an arm or wrist band a neckless or any shaped device.
6. The system according to any of the preceding aspects, wherein at least two of: the HCP EID external device,
   the patient EID external device,
   the HCP private key device, the patient private key device, and the DDI is configured for wireless communication using a standard network protocol.
7. The system according to any of the preceding aspects, wherein at least two of: the HCP EID external device,
   the patient EID external device,
   the HCP private key device, the patient private key device, and the DDI is configured for wireless communication using a proprietary network protocol.
8. The system according to any of the preceding aspects, wherein the patient EID external device is configured to use a first network protocol for communication with the implantable medical device and use a second network protocol for communication with the DDI.
9. The system according to any of the preceding aspects, wherein the patient EID external device is configured to use a first frequency band for communication with the implantable medical device and use a second frequency band for communication with the DDI.
10. The system according to any of the preceding aspects, wherein the DDI is configured to use a first frequency band for communication with the patient EID external device and a second frequency band for communication with the patient private key device.
11. The system according to any of the preceding aspects, wherein at least one of the HCP EID external device, the patient EID external device, the HCP private key device, the patient private key device and the DDI comprises a Bluetooth transceiver.
12. The system according to any of the preceding aspects, wherein at least one of the HCP EID external device, the patient EID external device, the HCP private key device, the patient private key device and the DDI comprises a UWB transceiver.
13. The system according to aspect 4, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
14. The system according to any of the preceding aspects, wherein the patient EID external device comprises a first wireless transceiver for wireless communication with the implantable medical device, and a second wireless transceiver for wireless communication with the DDI, and wherein the second wireless transceiver has longer effective range than the first wireless transceiver.
15. The system according to any of the preceding aspects, wherein the patient private key device comprises a first wireless transceiver for wireless communication with the HCP EID external device, and a second wireless transceiver for wireless communication with the DDI, and wherein the second wireless transceiver has longer effective range than the first wireless transceiver.
16. The system according to aspects 12 or 13, wherein the second wireless transceiver has an effective range being one of: 2 times, 4 times, 8 time, 20 times, 50 times or 100 times longer than the effective range of the first wireless transceiver.
17. The system according to any of aspects 12-14, wherein the second wireless transceiver is configured to be disabled to enable wireless communication using the first wireless transceiver.
18. The system according to any of the preceding aspects, wherein the patient EID external device is configured to allow transfer of data between the EID external device and the implantable medical device on the basis of an authentication of the patient EID external device.
19. The system according to any of the preceding aspects, wherein the patient EID external device is a wearable patient external device or a handset.
20. The system according to any of the preceding aspects, wherein the data encrypted by the implantable medical device is related to at least one of:
a battery status,
a temperature,
a time, or
an error.
21. A medical system comprising the system according to any one of the preceding aspects and an implantable medical device.
22. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
23. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and a main portion, wherein:
the first and second members are connected to the main portion, and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
24. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
25. The medical system according to aspect 21, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
26. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
27. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
28. The medical system according to aspect 21, wherein the implantable medical device comprises a medical device for actively stretching a stomach wall of a patient for creating a sensation of satiety, the medical device being implantable and comprising:
a first member comprising:
a first stomach engager configured to engage a first portion of the stomach wall, and
a second stomach engager portion configured to engage a second portion of the stomach wall,
a second member comprising:
a first stomach engager configured to engage a third portion of the stomach wall, and
a second stomach engager portion configured to engage a fourth portion of the stomach wall,
a main portion, wherein the first and second members are operably connected to the main portion,
an operation device comprising an actuator, wherein the
operation device is configured to displace the actuator, such that the actuator is configured to: in a first state, operate the first member for
displacing the first stomach engager of the first member in relation to the second stomach engager of the first member, for stretching the stomach wall, and
in a second state, operate the second member
for displacing the first stomach engager of the second member in relation to the second stomach engager of the second member, for stretching the stomach wall.
29. The medical system according to aspect 21, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
30. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
31. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member,
for stretching a portion of the stomach wall of the patient between the second and first member.
32. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient, and an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
33. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and
an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
34. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall,
a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
35. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a
second direction relative to at least one portion of the second member, and
move an operable third member in a third direction for stretching a portion of the stomach wall between the third and second members, and
a mechanical switching device configured to switch the operation device: from operating the first member to move in the first direction to operating the first member to move in the second direction, or
from operating the first member to move in the first direction to operating the third member to move in the third direction.
36. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
37. The medical system according to aspect 21, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. The medical system according to aspect 21, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
39. The medical system according to aspect 21, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
40. The medical system according to aspect 21, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein the stretching device is adapted to stretch a portion of the stomach wall between the first area and the second area.
41. The medical system according to aspect 21, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 397SE—eHealth_General_Communication_Dual
1. A system configured for changing pre-programmed treatment settings of an implantable medical device, when implanted in a patient, by a health care provider, HCP, in the physical presence of the patient, the system comprising:
at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and
allowed to perform said command by the HCP providing a HCP private key device comprising a HCP private key,
wherein the HCP private key device comprises at least one of:
a smart card,
a keyring device,
a watch,
a arm or wrist band,
a necklace, and
any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
wherein the HCP EID external device is adapted to be activated, authenticated, and allowed to perform said command also by the patient,
wherein the system further comprises:
a patient private key device comprising a patient private key, the patient private key device comprising at
least one of:
a smart card,
a keyring device,
a watch,
a arm or wrist band,
a necklace, and
any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted.
2. The system according to aspect 1, wherein the HCP EID external device further comprises a wireless transceiver configured for communication with the implanted medical device through a second network protocol.
3. The system according to aspect 1 or 2, wherein the HCP private key device is adapted to be provided to the at least one HCP external device via at least one of; a reading slot or comparable for the HCP private key device, a RFID communication, and a close distance wireless activation communication unit, or electrical direct contact.
4. The system according to anyone of the preceding aspects, wherein the HCP EID external device comprises at least one of: reading slot or comparable for the HCP private key device, a RFID communication and a close distance wireless activation communication unit, or electrical direct contact.
5. The system according to any of the preceding aspects, wherein the HCP EID external device is adapted to receive a command from a HCP dedicated device to change said pre-programmed treatment steps of the implantable medical device, when implanted, wherein the HCP dedicated device is further adapted to be activated, authenticated, and allowed to perform said command by the HCP providing their private key.
6. The system according to any of the preceding aspects, wherein at least two of: the HCP EID external device,
   the patient EID external device,
   the HCP private key device, and
the patient private key device,
are configured for wireless communication using a standard network protocol.
7. The system according to any of the preceding aspects, wherein at least two of:
the HCP EID external device,
   the patient EID external device,
   the HCP private key device, and
the patient private key device,
are configured for wireless communication using a proprietary network protocol.
8. The system according to any of the preceding aspects, wherein the patient EID external device is configured to use a first network protocol for communication with the implantable medical device and use a second network protocol for communication with the patient private key device.
9. The system according to any of the preceding aspects, wherein the patient EID external device is configured to use a first frequency band for communication with the implantable medical device and use a second frequency band for communication with the patient private key device.
10. The system according to any of the preceding aspects, wherein at least one of the HCP EID external device, the patient EID external device, the HCP private key device, and the patient private key device comprises a Bluetooth transceiver.
11. The system according to any of the preceding aspects, wherein at least one of the HCP EID external device, the patient EID external device, the HCP private key device, and the patient private key device comprises a UWB transceiver.
12. The system according to aspect 6, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
13. The system according to any of the preceding aspects, wherein the patient EID external device comprises a first wireless transceiver for wireless communication with the implantable medical device, and a second wireless transceiver for wireless communication with the patient private key device, and wherein the second wireless transceiver has longer effective range than the first wireless transceiver.
14. The system according to aspect 13, wherein the second wireless transceiver has an effective range being one of: 2 times, 4 times, 8 time, 20 times, 50 times or 100 times longer than the effective range of the first wireless transceiver.
15. The system according to aspects 13 or 14, wherein the second wireless transceiver is configured to be disabled to enable wireless communication using the first wireless transceiver.
16. The system according to any of the preceding aspects, wherein the patient EID external device is configured to allow transfer of data between the EID external device and the implantable medical device on the basis of an authentication of the patient EID external device.
17. The system according to any of the preceding aspects, wherein the patient EID external device is a wearable patient external device or a handset.
18. The system according to any of the preceding aspects, wherein the data encrypted by the implantable medical device is related to at least one of:
a battery status,
a temperature,
a time, or
an error.
19. The system according to any of the preceding aspects, comprising a master private key device configured to allow issuance of new private key device, wherein the HCP or HCP admin have such master private key device adapted to be able to replace and pair a new patient private key device or HCP private key device into the system, through the HCP EID external device.
20. The system according to anyone of the preceding aspects, wherein the patient remote external device and the patient EID external device are an integrated unit.
21. A medical system comprising the system according to any one of the preceding aspects and an implantable medical device.
22. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
23. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or
pivot when hydraulic fluid is moved to or from the fluid chamber for stretching the stomach wall between the first and second portions.
24. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
25. The medical system according to aspect 21, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion, an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
26. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to: receive a signal from the acoustic sensor, and
control the operation device on the basis of the signal received
from the acoustic sensor.
27. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
28. The medical system according to aspect 21, wherein the implantable medical device comprises a medical device for actively stretching a stomach wall of a patient for creating a sensation of satiety, the medical device being implantable and comprising:
   a first member comprising:
a first stomach engager configured to engage a first portion of the
stomach wall, and
a second stomach engager portion configured to engage a second
portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member
for displacing the first stomach engager of the second member in relation to the second stomach engager of the second member, for stretching the stomach wall.
29. The medical system according to aspect 21, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
30. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
31. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
32. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient, and an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
33. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and
an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
34. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
35. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall,
a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and move an operable third member in a third direction for
stretching a portion of the stomach wall between the third and second members, and a mechanical switching device configured to switch the operation device:
from operating the first member to move in the first direction to operating the first member to move in the second direction, or
from operating the first member to move in the first direction to operating the third member to move in the third direction.
36. The medical system according to aspect 21, wherein the implantable medical device comprises an implantable operation device for operating a body engaging portion of a medical implant, the implantable operation device comprising:
an electrical machine for transforming electrical energy to mechanical force, a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
37. The medical system according to aspect 21, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. The medical system according to aspect 21, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
39. The medical system according to aspect 21, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created, characterized in that the operable stretching device is adapted to be placed against the outside of the stomach wall.
40. The medical system according to aspect 21, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and the second engaging part is adapted to be engaged to a second area of the stomach wall, and wherein
   the stretching device is adapted to stretch a portion of the stomach wall between the first area and the second area.
41. The medical system according to aspect 21, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 398SE—eHealth_General_Communication_Dual
1. A system configured to change pre-programmed and pre-selected treatment actions of an implantable medical device, when implanted in a patient, by command from the patient, the system comprising:
an implantable medical device,
a patient remote external device, comprising
a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualised on the patient display device.
2. The system according to aspect 1, wherein the wireless transceiver, the remote display portal, and the remote display portal are comprised in the patient remote external device.
3. The system according to aspects 1 or 2, further comprising the patient display device comprising:
a supporting application,
a display which hosts the Remote Display Portal, and
a patient display device private key.
4. The system according to aspect 3, wherein the remote display portal is capable of generating a command to be signed by the patient display device private key.
5. The system according to anyone of the preceding aspects, wherein the patient remote external device is adapted to accept input from the patient via said patient display device through its remote display portal.
6. The system according to anyone of the preceding aspects, wherein the patient remote external device comprises a graphical user interface arranged on a touch-responsive display exposing buttons to express actuation functions of the implanted medical device.
7. The system according to anyone of the preceding aspects, configured to allow the patient to actuate the implant at home through the patient remote external device by means of an authorization granted by a patient private key.
8. The system according to aspect 7, wherein the patient private key comprises at least one of:
a smart card,
a keyring device,
a watch,
a arm or wrist band,
a necklace, and
any shaped device.
9. The system according to anyone of the preceding aspects, configured to allow the patient to actuate the implantable medical device, when implanted, at home through the patient remote external device, using an authorization granted by the patient private key.
10. The system according to anyone of the preceding aspects, wherein the system further comprises a patient EID external device comprising at least one of: a reading slot or comparable for the patient private key device, a RFID communication, and a close distance wireless activation communication, or electrical direct contact.
11. The system according to anyone of the preceding aspects, wherein the patient EID external device is adapted to be synchronised with the patient remote external device.
12. The system according to anyone of the preceding aspects, wherein said patient EID external device further comprises at least one of: a wireless transceiver configured for communication with the patient, a remote external device, and a wired connector for communication with the patient remote external device.
13. The system according to anyone of the preceding aspects, wherein the patient EID external device is adapted to generate an authorization to be signed by the patient private key to be installed into at least one of: the patient remote external device through the patient EID external device, and the implantable medical device.
14. The system according to anyone of the preceding aspect, comprising a patient display device comprising a supporting application capable of displaying the remote display portal with content delivered from the patient remote external device.
15. The system according to aspect 14, wherein said remote display portal and patient remote external device are adapted to expose buttons to express the will to actuate the functions of the implanted medical device by the patient through the patient remote external device.
16. The system according to aspect 14 or 15, wherein the patient display device comprises at least one of: a display which hosts the remote display portal, and a patient display device private key.
17. The system according to any of aspects 14-16, wherein said remote display portal is capable of generating a command to be signed by the patient private key.
18. The system according to any of the preceding aspects, comprising a master private key device configured to allow issuance of new private key device, wherein the HCP or HCP admin have such master private key device adapted to be able to replace and pair a new patient private key device or HCP private key device into the system, through the HCP EID external device.
19. The system according to anyone of the preceding aspects, wherein the patient remote external device and the patient EID external device are an integrated unit.
20. The system according to anyone of the preceding aspects, wherein the HCP dedicated device and the HCP EID external device are an integrated unit.
21. A medical system comprising the system according to any one of the preceding aspects and an implantable medical device.
22. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable
engagement member configured to engage at least the second member such that the eccentric rotation of the engagement member causes the second member to pivot in relation to the main portion for displacing the second member relative to the first member for stretching the stomach wall between the first and second portions.
23. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least
partially enclosed by a flexible wall portion, and
the flexible wall portion causes the second member to bend or
pivot when hydraulic fluid is moved to or from the fluid chamber for stretching the stomach wall between the first and second portions.
24. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
25. The medical system according to aspect 21, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
26. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   first direction relative to the first member, for stretching the stomach wall between the first and second portion,
   an acoustic sensor configured to sense at least one sound related to the patient swallowing, and
   a controller configured to:
receive a signal from the acoustic sensor, and control the operation device on the basis of the signal received from the acoustic sensor.
27. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
28. The medical system according to aspect 21, wherein the implantable medical device comprises a medical device for actively stretching a stomach wall of a patient for creating a sensation of satiety, the medical device being implantable and comprising:
   a first member comprising:
a first stomach engager configured to engage a first portion of the
stomach wall, and
a second stomach engager portion configured to engage a second
portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
in a first state, operate the first member for displacing the first stomach engager of the first member in relation to the second stomach engager of the first member, for stretching the stomach wall, and
   in a second state, operate the second member for displacing the first stomach engager of the second member in relation to the second stomach engager of the second member, for stretching the stomach wall.
29. The medical system according to aspect 21, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
30. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
31. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
32. The medical system according to aspect 21, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
33. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
34. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
35. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and
move an operable third member in a third direction for stretching a portion of the stomach wall between the third and second members, and
a mechanical switching device configured to switch the operation device:
from operating the first member to move in the first direction to operating the first member to move in the second direction, or
from operating the first member to move in the first direction to operating the third member to move in the third direction.
36. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
37. The medical system according to aspect 21, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. The medical system according to aspect 21, wherein the implantable medical device comprises a device for treating obesity of a patient, the device comprising:
at least one operable stretching device (10; 110) implantable in the patient and adapted to stretch a portion of the patient's stomach wall (12), and
an implantable control unit (42) for automatically controlling the operable stretching device, when the control unit and stretching device are implanted, to stretch the stomach wall portion in connection with the patient eating such that satiety is created,
characterized in that the stretching device comprises a first and a second engaging part (10″; 110a; 110b; 210a; 210b), wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
39. The medical system according to aspect 21, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
40. The medical system according to aspect 21, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
41. The medical system according to aspect 21, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 399SE—eHealth_General_Implant Information
1. A system configured for providing information from an implantable medical device, when implanted in a patient, to a distant remote location in relation to the patient, the system comprising:
at least one patient EID external device 320″′ adapted to receive information from the implant, adapted to send such information further on to a server or dedicated data infrastructure, DDI, 330, further adapted to be activated and authenticated and allowed to receive said information by the implanted medical device by the patient providing a private key,
a patient private key device comprising the private key adapted to be provided to the patient EID external device via at least one of: a reading slot or comparable for the patient private key device, a RFID communication or other close distance wireless activation communication or direct electrical connection, wherein said patient EID external device comprises at least one of;
a reading slot or comparable for the patient private key device,
an RFID communication, and
other close distance wireless activation communication or direct electrical contact; wherein said patient EID external device further comprises at least one wireless transceiver configured for communication with the DDI, through a first network protocol.
2. The system according to aspect 1, wherein the at least one patient EID external device 320″″ is adapted to receive information from the implant, through a second network protocol.
3. The system according to aspects 1 or 2, comprising the DDI, wherein the DD1 is adapted to receive information from said patient EID external device, and wherein the DDI comprises a wireless transceiver configured for communication with said patient EID external device.
4. The system according to anyone of the preceding aspects, wherein the patient EID external device is adapted to receive a command relayed by the DDI, to further send the command to the implanted medical device to change said pre-programmed treatment settings of the implanted medical device, and further adapted to be activated and authenticated and allowed to perform said command by the patient providing the patient private key.
5. The system according to anyone of the preceding aspects, wherein the patient private key device is adapted to provide the patient private key to the patient EID external device by the patient via at least one of; a reading slot or comparable for the patient private key device, an RFID communication or other close distance wireless activation communication, or electrical direct contact.
6. The system according to anyone of the preceding aspects, said patient EID external device comprising at least one of: a reading slot or comparable for the HCP private key device, a RFID communication, and other close distance wireless activation communication, or direct electrical contact.
7. The system according to anyone of the preceding aspects, said patient EID external device further comprising at least one wireless transceiver configured for communication with the implanted medical device through a second network protocol.
8. The system according to anyone of the preceding aspects, comprising the implantable medical device adapted to, when implanted, treat the patient or perform a bodily function.
9. The system according to anyone of the preceding aspect, wherein the patient private key comprises at least one of:
a smart card,
a keyring device,
a watch,
an arm band or wrist band,
a necklace, and
any shaped device.
10. The system according to any of the preceding aspects, wherein at least two of:
the patient EID external device,
the IDD, and
the patient private key device,
are configured for wireless communication using a standard network protocol.
11. The system according to any of the preceding aspects, wherein at least two of:
the patient EID external device,
the IDD, and
the patient private key device,
are configured for wireless communication using a proprietary network protocol.
12. The system according to any of the preceding aspects, wherein the patient EID external device is configured to use a first network protocol for communication with the implantable medical device and use a second network protocol for communication with the patient private key device.
13. The system according to any of the preceding aspects, wherein the patient EID external device is configured to use a first frequency band for communication with the implantable medical device and use a second frequency band for communication with the patient private key device.
14. The system according to any of the preceding aspects, wherein at least one of the patient EID external device, the patient private key device and the IDD comprises a Bluetooth transceiver.
15. The system according to any of the preceding aspects, wherein at least one of the patient EID external device, the patient private key device and the IDD comprises a UWB transceiver.
16. The system according to aspect 10, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
17. The system according to any of the preceding aspects, wherein the patient EID external device comprises a first wireless transceiver for wireless communication with the implantable medical device, and a second wireless transceiver for wireless communication with the patient private key device, and wherein the second wireless transceiver has longer effective range than the first wireless transceiver.
18. The system according to aspect 17, wherein the second wireless transceiver has an effective range being one of: 2 times, 4 times, 8 time, 20 times, 50 times or 100 times longer than the effective range of the first wireless transceiver.
19. The system according to aspects 17 or 18, wherein the second wireless transceiver is configured to be disabled to enable wireless communication using the first wireless transceiver.
20. The system according to any of the preceding aspects, wherein the patient EID external device is a wearable patient external device or a handset.
21. A medical system comprising the system according to any one of the preceding aspects and an implantable medical device.
22. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
23. The medical system according to aspect 21, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
24. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
25. The medical system according to aspect 21, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion, an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
26. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
27. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
28. The medical system according to aspect 21, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second
portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
an operation device comprising an actuator, wherein the operation device is configured to displace the actuator, such that the actuator is configured to: in a first state, operate the first member for
displacing the first stomach engager of the first member in relation to the second stomach engager of the first member, for stretching the stomach wall, and
in a second state, operate the second member
for displacing the first stomach engager of the second member in relation to the second stomach engager of the second member, for stretching the stomach wall.
29. The medical system according to aspect 21, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
30. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
31. The medical system according to aspect 21, wherein the implantable medical device comprises a medical device for actively stretching a stomach wall of a patient for creating a sensation of satiety, the medical device being implantable and comprising: a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
32. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient, and an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
33. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
34. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
35. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and
move an operable third member in a third direction for stretching a portion of the stomach wall between the third and second members, and
a mechanical switching device configured to switch the operation device: from operating the first member to move in the first direction to operating the first member to move in the second direction, or
from operating the first member to move in the first direction to operating the third member to move in the third direction.
36. The medical system according to aspect 21, wherein the implantable medical device comprises an implantable operation device for operating a body engaging portion of a medical implant, the implantable operation device comprising:
an electrical machine for transforming electrical energy to mechanical force,
a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
37. The medical system according to aspect 21, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. The medical system according to aspect 21, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
39. The medical system according to aspect 21, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
40. The medical system according to aspect 21, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and the second engaging part is adapted to be engaged to a second area of the stomach wall, and wherein
   the stretching device is adapted to stretch a portion of the stomach wall between the first area and the second area.
41. The medical system according to aspect 21, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 400SE—eHealth_General_system, DDI inactivation of remote or private key
1. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key.
2. The system according to aspect 1, wherein the at least one patient remote external device comprises a patient remote external device private key, wherein the DDI via the patient EID external device is able to inactivate the authority and authenticating function of the patient remote external device, thereby inactivating the patient remote external device.
2. The system according to aspect 1 or 2, wherein said patient EID external device comprises at least one wireless transceiver configured for communication with the DD1 via a first network protocol.
3. The system according to anyone of the preceding aspects, comprising the DDI, wherein the DDI is adapted to receive command from a HCP EID external device, and to send the received command to the patient EID external device, wherein the DDI comprises a wireless transceiver configured for communication with said patient external device.
4. The system according to anyone of the preceding aspects, wherein the patient EID external device is adapted to receive the command from the DDI, wherein the command originates from a health care provider, HCP, and wherein the patient EID is adapted to inactivate the patient private key and to send the command to the implanted medical device.
5. The system according to anyone of the preceding aspects, wherein the patient EID external device is adapted to receive the command from the DDI, wherein the command originates from a health care provider, HCP, wherein the patient EID external device is adapted to receive the command from the HCP via the DDI to inactivate the patient remote external device comprising a patient remote external device private key, and wherein the patient EID external device is further adapted to send this command to the implanted medical device.
6. The system according to anyone of the preceding aspects, wherein the patient EID external device further comprises at least one wireless transceiver configured for communication with the implanted medical device through a second network protocol.
7. The system according to aspect any preceding aspect, wherein at least one of the patient private key and a patient remote external device private key comprises a hardware key.
8. The system according to any preceding aspect, wherein the private key device is at least one of, a smartcard, a key-ring device, a watch an arm or wrist band a neckless or any shaped device.
9. The system according to any preceding aspect, wherein at least two of:
the patient remote external device,
the patient EID external device,
the patient private key device, and
the DDI,
are configured for wireless communication using a standard network protocol.
10. The system according to any preceding aspect, wherein at least two of:
the patient remote external device,
the patient EID external device,
the patient private key device, and
the DDI,
are configured for wireless communication using a proprietary network protocol.
11. The system according to any of the preceding aspects, wherein the patient EID external device is configured to use a first network protocol for communication with the implantable medical device and use a second network protocol for communication with the patient private key device.
12. The system according to any of the preceding aspects, wherein the patient EID external device is configured to use a first frequency band for communication with the implantable medical device and use a second frequency band for communication with the patient private key device.
13. The system according to any of the preceding aspects, wherein at least one of the patient remote external device, the patient EID external device, the patient private key device, and the DDI, comprise a Bluetooth transceiver.
14. The system according to any of the preceding aspects, wherein at least one of the patient remote external device, the patient EID external device, the patient private key device, and the DDI, comprise an UWB transceiver.
15. The system according to aspect 9, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
16. The system according to any of the preceding aspects, patient EID external device comprises a first wireless transceiver for wireless communication with the implantable medical device, and a second wireless transceiver for wireless communication with the patient private key device, and wherein the second wireless transceiver has longer effective range than the first wireless transceiver.
17. The system according to aspect 16, wherein the second wireless transceiver has an effective range being one of: 2 times, 4 times, 8 time, 20 times, 50 times or 100 times longer than the effective range of the first wireless transceiver.
18. The system according to aspects 16 or 17, wherein the second wireless transceiver is configured to be disabled to enable wireless communication using the first wireless transceiver.
19. The system according to any of the preceding aspects, wherein the patient EID external device is a wearable patient external device or a handset.
20. The system according to any of the preceding aspects, wherein the data encrypted by the implantable medical device is related to at least one of:
a battery status,
a temperature,
a time, or
an error.
21. A medical system comprising the system according to any one of the preceding aspects and an implantable medical device.
22. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
23. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion, and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
24. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
25. The medical system according to aspect 21, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
26. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to: receive a signal from the acoustic sensor, and
control the operation device on the basis of the signal received from the acoustic sensor.
27. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and a controller configured to:
receive a signal from the sensor, and
control the operation device on the basis of the signal received from the sensor.
28. The medical system according to aspect 21, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
29. The medical system according to aspect 21, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
30. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises: a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
31. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
32. The medical system according to aspect 21, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
33. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
34. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
35. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and move an operable third member in a third direction for stretching a portion of the stomach wall between the third and second members, and
a mechanical switching device configured to switch the operation device:
from operating the first member to move in the first direction to operating the first member to move in the second direction, or
from operating the first member to move in the first direction to operating the third member to move in the third direction.
36. The medical system according to aspect 21, wherein the implantable medical device comprises an implantable operation device for operating a body engaging portion of a medical implant, the implantable operation device comprising:
an electrical machine for transforming electrical energy to mechanical force, a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
37. The medical system according to aspect 21, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. The medical system according to aspect 21, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
39. The medical system according to aspect 21, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
40. The medical system according to aspect 21, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
41. The medical system according to aspect 21, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 401SE—eHealth_General_Patient_at Hospital
1. A system configured for changing pre-programmed treatment settings in steps of an implantable medical device, when implanted in a patient, by a health care provider, HCP, either in the physical presence of the patient or remotely with the patient on distance, the system comprising:
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
a arm or wrist band,
a necklace, and
any shaped device;
wherein the system further comprises:
a patient private key device comprising a patient private key, comprising at least one of:
a smart card,
a keyring device,
a watch,
a arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device.
2. The system according to anyone of the preceding aspects, comprising a master private key device that allow issuance of new private key device wherein the HCP or HCP admin have such master private key device adapted to be able to replace and pair a new patient private key device or HCP private key device into the system, through the HCP EID external device.
3. The system according to anyone of the preceding aspects, wherein the patient remote external device and the patient EID external device are an integrated unit.
4. The system according to anyone of the preceding aspects, wherein the HCP dedicated device and the HCP EID external device are an integrated unit.
5. The system according to anyone of the preceding aspects, further comprising a measurement device or sensor adapted to deliver a measurement to at least one of the DDI, patent EID external device and a patient display device.
6. The system according to anyone of the preceding aspects, further comprising a food sensor adapted to measure at least if the patient swallows solid food or is drinking fluid, wherein said food sensor is configured to be connected to the control unit of a medical device to cause an action to stretch the stomach after a determined amount of food intake.
7. The system according to any preceding aspect, wherein the HCP EID external device further comprises a wireless transceiver configured for communication with the implanted medical device through a second network protocol.
8. The system according to aspect any preceding aspect, wherein the HCP private key device is adapted to be provided to the at least one HCP external device via at least one of;
a reading slot or comparable for the HCP private key device, a RFID communication, and
a close distance wireless activation communication unit, or electrical direct contact.
9. The system according to anyone of the preceding aspects, wherein the HCP EID external device comprises at least one of:
reading slot or comparable for the HCP private key device,
a RFID communication, and
a close distance wireless activation communication unit, or electrical direct contact.
10. The system according to any of the preceding aspects, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
11. The system according to any of the preceding aspects, wherein the HCP EID external device and the HCP private key device are configured for wireless communication using a standard network protocol.
12. The system according to any of the preceding aspects, wherein the HCP EID external device and the HCP private key device are configured for wireless communication using a proprietary network protocol.
13. The system according to any of the preceding aspects, wherein the HCP EID external device is configured to use a first network protocol for communication with the implantable medical device and use a second network protocol for communication with the HCP private key device.
14. A medical system comprising the system according to any one of the preceding aspects and an implantable medical device.
15. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
16. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
17. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
18. The medical system according to aspect 14, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
19. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
20. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
21. The medical system according to aspect 14, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
22. The medical system according to aspect 14, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
23. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
24. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
25. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
26. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
27. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
28. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
29. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
30. The medical system according to aspect 14, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. The medical system according to aspect 14, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
32. The medical system according to aspect 14, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
33. The medical system according to aspect 14, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. The medical system according to aspect 14, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 402SE—eHealth_General_EID and DDI
1. A system configured for changing pre-programmed treatment settings in steps of an implantable medical device, when implanted in a patient, by a health care provider, HCP, with the patient on remote on distance, the system comprising:
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device.
2. The system according to aspect 1, comprising the HCP private key device comprising a HCP private key, comprising at least one of:
a smart card,
a keyring device,
a watch,
a arm or wrist band,
a necklace, and
any shaped device.
3. The system according to anyone of the preceding aspects, comprising: the patient private key device comprising a patient private key, comprising at least one of:
a smart card,
a keyring device,
a watch,
a arm or wrist band,
a necklace, and
any shaped device.
5. The system according to anyone of the preceding aspects, wherein the patient private key is adapted to activate, be authenticated, and allowed to perform said command provided by the HCP, either via the HCP EID external device or when the action is performed remotely via a patient EID external device.
6. The system according to anyone of the preceding aspects, further comprising a dedicated data infrastructure, DDI, the patient EID external device, and the HCP EID external device, wherein the communication between the patient EID external device and the HCP EID external device is performed via the DDI.
7. The system according to anyone of the preceding aspects, comprising a master private key device that allows issuance of new private key device wherein the HCP or HCP admin have such master private key device adapted to be able to replace and pair a new patient private key device or HCP private key device into the system.
8. The system according to anyone of the preceding aspects, wherein the patient remote external device and the patient EID external device are an integrated unit.
10. The system according to anyone of the preceding aspects, wherein the HCP dedicated device and the HCP EID external device are an integrated unit.
11. The system according to anyone of the preceding aspects, further comprising a measurement device or sensor adapted to deliver a measurement to at least one of the DDI, patent EID external device and patient display device.
12. The system according to anyone of the preceding aspects, comprising a food sensor, adapted to measure at least if the patient swallow solid food or is drinking fluid, wherein said food sensor is connected to the control unit of a medical device to cause an action to stretch the stomach after a determined amount of food intake.
13. The system according to any preceding aspect, wherein the HCP EID external device further comprises a wireless transceiver configured for communication with the implanted medical device through a second network protocol.
14. The system according to aspect any preceding aspect, wherein the HCP private key device is adapted to be provided to the at least one HCP external device via at least one of: a reading slot or comparable for the HCP private key device, a RFID communication, and a close distance wireless activation communication unit, or electrical direct contact.
15. The system according to anyone of the preceding aspects, wherein the HCP EID external device comprises at least one of: reading slot or comparable for the HCP private key device, a RFID communication, and a close distance wireless activation communication unit, or electrical direct contact.
16. The system according to any of the preceding aspects, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
17. The system according to any of the preceding aspects, wherein the HCP EID external device and the HCP private key device are configured for wireless communication using a standard network protocol.
18. The system according to any of the preceding aspects, wherein the HCP EID external device and the HCP private key device are configured for wireless communication using a proprietary network protocol.
19. The system according to any of the preceding aspects, wherein the HCP EID external device is configured to use a first network protocol for communication with the implantable medical device and use a second network protocol for communication with the HCP private key device.
20. The system according to any of the preceding aspects, wherein the HPC EID external device is configured to use a first frequency band for communication with the implantable medical device and use a second frequency band for communication with the HCP private key device.
21. A medical system comprising the system according to any one of the preceding aspects and an implantable medical device.
22. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
23. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion, at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
24. The medical system according to aspect 21, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   first direction, for stretching the stomach wall between the first and second portions, and a main portion, wherein:
the first and second members are connected to the main portion,
at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
25. The medical system according to aspect 21, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
26. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received
from the acoustic sensor.
27. The medical system according to aspect 21, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
28. The medical system according to aspect 21, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
29. The medical system according to aspect 21, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
30. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
31. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member,
for stretching a portion of the stomach wall of the patient between the second and first member.
32. The medical system according to aspect 21, wherein the implantable medical device comprises a medical device for actively stretching a stomach wall of a patient for creating a sensation of satiety, the medical device being implantable and comprising:
a second member configured to be fixated to the stomach wall of the patient, and an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
33. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
34. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first
direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
35. The medical system according to aspect 21, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and move an operable third member in a third direction for
stretching a portion of the stomach wall between the third and second members, and
   a mechanical switching device configured to switch the operation device:
from operating the first member to move in the first direction to operating the first member to move in the second direction, or
from operating the first member to move in the first direction to operating the third member to move in the third direction.
36. The medical system according to aspect 21, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch
the force exerted on shaft such that:
in a first state, a rotational force is exerted on the shaft, and in a second, a linear force is exerted in the shaft.
37. The medical system according to aspect 21, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   38. The medical system according to aspect 21, wherein the implantable medical device comprises a device for treating obesity of a patient, the device comprising:
at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein the stretching device is adapted to stretch a portion of the stomach wall between the first area and the second area.
39. The medical system according to aspect 21, wherein the implantable medical device comprises an obesity treatment device comprising:
at least one operable stretching device (10; 10″; 110a; 110b; 410b; 50; 810) implantable in a patient and adapted to stretch a portion of the patient's stomach wall (12) and
an implantable operation device (90; 42; 54; 40; 217; 218; 452) for operating the stretching device, when implanted, to stretch the stomach wall portion such that satiety is created, characterized in that the operable stretching device is adapted to be placed against the outside of the stomach wall.
40. The medical system according to aspect 21, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
41. The medical system according to aspect 21, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 403SE—eHealth_General
1. A system configured for changing pre-programmed treatment settings of an implantable medical device, when implanted in a patient, from a distant remote location in relation to the patient, the system comprising:
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device, a RFID communication, and
other close distance wireless activation communication or electrical direct contact, wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.
2. The system according to aspect 1, wherein at least one of the patient private key device or HCP private key device comprises a hardware key.
3. The system according to aspect 1 or 2, wherein the private key device is at least one of, a smartcard, a key-ring device, a watch an arm or wrist band a neckless or any shaped device.
4. The system according to anyone of the preceding aspects, comprising a master private key device that allow issuance of new private key device wherein the HCP or HCP admin have such master private key device adapted to be able to replace and pair a new patient private key device or HCP private key device into the system, through the HCP EID external device.
5. The system according to anyone of the preceding aspects, wherein the patient remote external device and the patient EID external device is an integrated unit.
6. The system according to anyone of the preceding aspects, wherein the HCP dedicated device and the HCP EID external device are an integrated unit.
7. The system according to anyone of the preceding aspects, comprising a measurement device or sensor adapted to deliver a measurement to at least one of the DDI, patent EID external device and a patient display device.
8. The system according to anyone of the preceding aspects, comprising a food sensor, adapted to measure at least if the patient swallow solid food or is drinking fluid, wherein said food sensor is connected to the control unit of a medical device to cause an action to stretch the stomach after a determined amount of food intake.
9. The system according to any preceding aspect, wherein the HCP EID external device further comprises a wireless transceiver configured for communication with the implanted medical device through a second network protocol.
10. The system according to aspect any preceding aspect, wherein the HCP private key device is adapted to be provided to the at least one HCP external device via at least one of;
a reading slot or comparable for the HCP private key device,
a RFID communication, and
a close distance wireless activation communication unit, or electrical direct contact.
11. The system according to anyone of the preceding aspects, wherein the HCP EID external device comprises at least one of:
reading slot or comparable for the HCP private key device,
a RFID communication, and
a close distance wireless activation communication unit, or electrical direct contact.
12. The system according to any of the preceding aspects, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
13. The system according to any of the preceding aspects, wherein the HCP EID external device and the HCP private key device are configured for wireless communication using a standard network protocol.
14. A medical system comprising the system according to any one of the preceding aspects and an implantable medical device.
15. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
16. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
17. The medical system according to aspect 14, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
18. The medical system according to aspect 14, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
19. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
20. The medical system according to aspect 14, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
21. The medical system according to aspect 14, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
22. The medical system according to aspect 14, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
23. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
24. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
25. The medical system according to aspect 14, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
26. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
27. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
28. The medical system according to aspect 14, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
29. The medical system according to aspect 14, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
30. The medical system according to aspect 14, wherein the implantable medical device comprises an apparatus for treating obesity of a patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. The medical system according to aspect 14, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
32. The medical system according to aspect 14, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
33. The medical system according to aspect 14, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. The medical system according to aspect 14, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with
      Aspect 330SE eHealth General Communication Housing
1. An external device configured for communication with an implantable medical device implanted in a patient, the external device comprising:
   a display device,
   a housing unit configured to mechanically, disconnectably connect to the display device, the housing unit comprising:
   a first communication unit for receiving communication from the display device, and
   a second communication unit for wirelessly transmitting communication to the implantable medical device.
2. The external device according to aspect 1, wherein the external device comprises a handheld electronic device.
3. The external device according to any one of aspects 1 and 2, wherein the external device is configured for communicating with the implantable medical device for changing an operational state of the implantable medical device.
4. The external device according to any one of the preceding aspects, wherein the first communication unit is a wireless communication unit for wireless communication with the display device.
5. The external device according to aspect 4, wherein: the first communication unit is configured to communicate wirelessly with the display device using a first communication frequency, the second communication unit is configured to communicate wirelessly with the implantable medical device (10) using a second communication frequency, and the first and second communication frequencies are different.
6. The external device according to any one of the preceding aspects, wherein the second communication unit is configured to communicate wirelessly with the implantable medical device (10) using electromagnetic waves at a frequency below 100 kHz.
7. The external device according to any one of the preceding aspects, wherein the second communication unit is configured to communicate wirelessly with the implantable medical device (10) using electromagnetic waves at a frequency below 40 kHz.
8. The external device according to any one of aspects 4-7, wherein the first communication unit is configured to communicate wirelessly with the display device using electromagnetic waves at a frequency above 100 kHz.
9. The external device according to any one of the preceding aspects, wherein: the first communication unit is configured to communicate with the display device using a first communication protocol,
the second communication unit is configured to communicate wirelessly with the implantable medical device (10) using a second communication protocol, and the first and second communication protocols are different.
10. The external device according to any one of aspects 3-9, wherein the housing unit comprises:
a first antenna configured for wireless communication with the display device, and
a second antenna configured for wireless communication with the implantable medical device (10).
11. The external device according to any one of aspects 1-3, wherein the first communication unit is a wire-based communication unit for wire-based communication with the display device.
12. The external device according to any one of the preceding aspects, wherein the display device comprises: a first communication unit for communication with the housing unit, and a second communication unit for wireless communication with a second external device.
13. The external device according to aspect 12, wherein the second communication unit of the display device is configured for communicating with the second external device over the Internet.
14. The external device according to any one of aspects 12 and 13, wherein the first communication unit of the display device is a wireless communication unit for wireless communication with the housing unit.
15. The external device according to aspect 14, wherein: the first communication unit of the display device is configured to communicate wirelessly with the housing unit using a first communication frequency, the second communication unit of the display device is configured to communicate wirelessly with the second external device using a second communication frequency, and the first and second communication frequencies are different.
16. The external device according to any one of aspects 14 and 15, wherein: the first communication unit of the display device is configured to communicate wirelessly with the housing unit using a first communication protocol, the second communication unit of the display device is configured to communicate wirelessly with the second external device using a second communication protocol, and the first and second communication protocols are different.
17. The external device according to any one of aspects 14-16, wherein the display device comprises: a first antenna configured for wireless communication with the housing, and a second antenna configured for wireless communication with the second external device.
18. The external device according to any one of aspects 12-13, wherein the first communication unit is a wire-based communication unit for wire-based communication with the housing unit.
19. The external device according to any one of the preceding aspects, wherein the display device is configured to display a user interface to the patient.
20. The external device according to any one of the preceding aspects, wherein the housing unit is configured to transmit information pertaining to the display of the user interface to the display device.
21. The external device according to any one of aspects 19 and 20, wherein the display device is configured to: receive input pertaining to communication to or from the implantable medical device from the patient, and transmit communication based on the received input to the housing unit.
22. The external device according to any one of aspects 19-21, wherein the display device comprises a touch screen configured to display the user interface and receive the input from the patient.
23. The external device according to any one of the preceding aspects, wherein the housing unit is configured to display a user interface to the patient.
24. The external device according to any one of the preceding aspects, wherein the first communication unit of the housing unit is configured to receive communication from the implantable medical device pertaining to input from the patient, and wirelessly transmit communication based on the received input to the implantable medical device, using the second communication unit.
25. The external device according to any one of the preceding aspects, wherein the second communication unit of the housing unit is configured for wireless communication with the implantable medical device using a standard network protocol.
26. The external device according to aspect 25, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
27. The external device according to aspect 25, wherein the second communication unit of the housing unit comprises a Bluetooth transceiver.
27. The external device according to any one of the preceding aspects, wherein the second communication unit of the housing unit is configured for wireless communication with the implantable medical device using a proprietary network protocol.
28. The external device according to any one of aspects 25-27, wherein the second communication unit of the housing unit comprises a UWB transceiver.
29. The external device according to any one of aspects 4-28, wherein the first communication unit of the housing unit is configured for wireless communication with the display device using a standard network protocol.
30. The external device according to aspect 29, wherein the standard network protocol is an NFC type protocol.
31. The external device according to any one of aspects 4-28, wherein the first communication unit of the housing unit is configured for wireless communication with the display device using a proprietary network protocol.
32. The external device according to any one of aspects 4-31, wherein a communication range of the first communication unit of the housing unit is less than a communication range of the second communication unit of the housing unit.
33. The external device according to any one of aspects 14-32, wherein a communication range of the first communication unit of the display device is less than a communication range of the second communication unit of the display device.
34. The external device according to any one of the preceding aspects, wherein at least one of the housing unit and the display device is configured allow communication between the housing unit and the display device on the basis of a distance between the housing unit and the display device.
35. The external device according to any one of the preceding aspects, wherein at least one of the housing unit and the display device is configured allow communication between the housing unit and the display device on the basis of the housing unit being mechanically connected to the display device.
36. The external device according to any one of the preceding aspects, wherein the housing unit is configured allow communication between the housing unit and the implantable medical device on the basis of a distance between the housing unit and the implantable medical device.
37. The external device according to any one of the preceding aspects, wherein the housing unit further comprises an encryption unit configured to encrypt communication received from the display device.
38. The external device according to aspect 37, wherein the housing unit is further adapted to transmit the encrypted communication, using the second communication unit, to the implantable medical device.
39. The external device according to any one of aspects 14-38, wherein the second communication unit of the display device is configured to be disabled to enable at least one of: communication between the display device and the housing unit, and communication between the housing unit and the implantable medical device.
40. The external device according to any one of the preceding aspects, wherein the display device is a wearable device or a handset.
41. A medical system comprising the external device according to any one of the preceding aspects and an implantable medical device.
42. The medical system according to aspect 41, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion, and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
43. The medical system according to aspect 41, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least
partially enclosed by a flexible wall portion, and
the flexible wall portion causes the second member to bend or pivot when hydraulic fluid is moved to or from the fluid chamber for stretching the stomach wall between the first and second portions.
44. The medical system according to aspect 41, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion,
the operation device comprises a first flexible cable for
pulling on the second member for displacing the second member in the direction away from the first member, and
the first flexible cable is fixated to the second member at the distal half of thereof.
45. The medical system according to aspect 41, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
46. The medical system according to aspect 41, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received
from the acoustic sensor.
47. The medical system according to aspect 41, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
48. The medical system according to aspect 41, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
49. The medical system according to aspect 41, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
50. The medical system according to aspect 41, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
51. The medical system according to aspect 41, wherein the implantable medical device comprises a medical device for actively stretching a stomach wall of a patient for creating a sensation of satiety, the
medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member,
for stretching a portion of the stomach wall of the patient between the second and first member.
52. The medical system according to aspect 41, wherein the implantable medical device comprises a medical device for actively stretching a stomach wall of a patient for creating a sensation of satiety, the medical device being implantable and comprising:
a second member configured to be fixated to the stomach wall of the patient, and
an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
53. The medical system according to aspect 41, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and
an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
54. The medical system according to aspect 41, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall,
a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
55. The medical system according to aspect 41, wherein the implantable medical device comprises a medical device for actively stretching a stomach wall of a patient for creating a sensation of satiety, the medical device being implantable and comprising:
an operable first member configured to be fixated to a first portion of the stomach wall,
a second member configured to be fixated to a second portion of the stomach wall,
an operation device configured to:
move at least a portion of the first member in a first direction relative to at least one portion of the second member, for stretching a portion of the stomach wall between the first and second members, and at least one of:
move at least a portion of the operable first member in a second direction relative to at least one portion of the second member, and
move an operable third member in a third direction for stretching a portion of the stomach wall between the third and second members, and a mechanical switching device configured to switch the operation device:
from operating the first member to move in the first direction to operating the first member to move in the second direction, or
from operating the first member to move in the first direction to operating the third member to move in the third direction.
56. The medical system according to aspect 41, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
57. The medical system according to aspect 41, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
58. The medical system according to aspect 41, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 410a; 410b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
59. The medical system according to aspect 41, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 417; 418; 452) for operating the stretching device, when implanted, to stretch the stomach wall portion such that satiety is created,
   characterized in that the operable stretching device is adapted to be placed against the outside of the stomach wall.
60. The medical system according to aspect 41, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein the stretching device is adapted to stretch a portion of the stomach wall between the first area and the second area.
61. The medical system according to aspect 41, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 331SE eHealth General Security Module
1. An implantable controller for an implantable medical device, the implantable controller comprises:
a wireless transceiver for communicating wirelessly with an external device, a security module, and
a central unit configured to be in communication with the wireless transceiver, the security module and the implantable medical device:
the wireless transceiver is configured to receive communication from the external device including at least one instruction to the implantable medical device, and transmit the received communication to the central unit,
the central unit is configured to send secure communication to the security module, derived from the received communication from the external device, and
the security module is configured to at least one of:
decrypt at least a portion of the secure communication, and
verify the authenticity of the secure communication, and
the security module is configured to transmit a response communication to the central unit, and the central unit is configured to communicate the at least one instruction to the implantable medical device, the at least one instruction being based on:
the response communication, or
a combination of the response communication and the received communication from the external device.
2. The implantable controller according to aspect 1, wherein the security module comprises a set of rules for accepting communication from the central unit.
3. The implantable controller according to aspect 2, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
4. The implantable controller according to aspect 4, wherein the set of rules comprises a rule stipulating that communication from the central unit is only accepted when the wireless transceiver has been placed in the off-mode for a specific time period.
5. The implantable controller according to any one of the preceding aspects wherein the central unit is configured to verify a digital signature of the received communication from the external device.
6. The implantable controller according to aspect 4, wherein the set of rules comprises a rule stipulating that communication from the central unit is only accepted when the digital signature of the received communication has been verified by the central unit.
7. The implantable controller according to any one of the preceding aspects, wherein the central unit is configured to verify the size of the received communication from the external device.
8. The implantable controller according to aspect 7, wherein the set of rules comprises a rule stipulating that communication from the central unit is only accepted when the size of the received communication has been verified by the central unit.
9. The implantable controller according to any one of the preceding aspects, wherein: the wireless transceiver is configured to receive a message from the external device being encrypted with at least a first and second layer of encryption, the central unit is configured to decrypt a first layer of decryption and transmit at least a portion of the message comprising the second layer of encryption to the security model, and the security module is configured to decrypt the second layer of encryption and transmit a response communication to the central unit based on the portion of the message decrypted by the security module.
10. The implantable controller according to aspect 9, wherein the central unit is configured to decrypt a portion of the message comprising a digital signature, such that the digital signature can be verified by the central unit.
11. The implantable controller according to aspect 9, wherein the central unit is configured to decrypt a portion of the message comprising message size information, such that the message size can be verified by the central unit.
12. The implantable controller according to aspect 9, wherein the central unit is configured to decrypt a first and second portion of the message, and wherein the first portion comprises a checksum for verifying the authenticity of the second portion.
13. The implantable controller according to any one of aspects 9-12, wherein the response communication transmitted from the security module comprises a checksum, and wherein the central unit is configured to verify the authenticity of at least a portion of the message decrypted by the central unit using the received checksum.
14. The implantable controller according to aspect 4, wherein the set of rules comprises a rule related to the rate of data transfer between the central unit and the security module.
15. The implantable controller according to any one of aspects 9-14, wherein the security module is configured to decrypt a portion of the message comprising a digital signature, encrypted with the second layer of encryption, such that the digital signature can be verified by the security module.
16. The implantable controller according to any one of aspects 4-15, wherein the central unit is only capable of decrypting a portion of the receive communication from the external device when the wireless transceiver is placed in the off-mode.
17. The implantable controller according to any one of aspects 4-16, wherein the central unit is only capable of communicating the at least one instruction to the implantable medical device when the wireless transceiver is placed in the off-mode.
18. The implantable controller according to any one of the preceding aspects, wherein the implantable controller is configured to: receive, using the wireless transceiver, a message from the external device comprising a first un-encrypted portion and a second encrypted portion, decrypt the encrypted portion, and use the decrypted portion to verify the authenticity of the un-encrypted portion.
19. The implantable controller according to aspect 18, wherein the central unit is configured to: transmit the encrypted portion to the security module, receive a response communication from the security module, based on information contained in the encrypted portion being decrypted by the security module, and use the response communication to verify the authenticity of the un-encrypted portion.
20. The implantable controller according to any one of aspects 18-19, wherein the un-encrypted portion comprises at least a portion of the at least one instruction to the implantable medical device.
21. The implantable controller according to any one of the preceding aspects, wherein the implantable controller is configured to: receive, using the wireless transceiver, a message from the external device comprising information related to at least one of: a physiological parameter of the patient and a physical parameter of the implanted medical device, and use the received information to verify the authenticity of the message.
22. The implantable controller according to aspect 21, wherein the physiological parameter of the patient comprises at least one of: a temperature, a heart rate and a saturation value.
23. The implantable controller according to aspect 21, wherein the physical parameter of the implanted medical device comprises at least one of: a current setting or value of the implanted medical device, a prior instruction sent to the implanted medical device or an ID of the implanted medical device.
24. The implantable controller according to any one of aspects 21-23, wherein the portion of the message comprising the information is encrypted, and wherein the central unit is configured to transmit the encrypted portion to the security module and receive a response communication from the security module, based on the information having been decrypted by the security module.
25. The implantable controller according to any one of the preceding aspects, wherein the security module comprises a hardware security module comprising at least one hardware-based key.
26. The implantable controller according to aspect 25, wherein the hardware-based key corresponds to a hardware-based key in the external device.
27. The implantable controller according to aspect 25, wherein the hardware-based key corresponds to a hardware-based key on a key-card connectable to the external device.
28. The implantable controller according to any one of the preceding aspects, wherein the security module comprises a software security module comprising at least one software-based key.
29. The implantable controller according to aspect 28, wherein the software-based key corresponds to a software-based key in the external device.
30. The implantable controller according to aspect 28, wherein the software-based key corresponds to a software-based key on a key-card connectable to the external device.
31. The implantable controller according to any one of the preceding aspects, wherein the security module comprises a combination of a software-based key and a hardware-based key.
32. The implantable controller according to any one of the preceding aspects, wherein the security module comprises at least one cryptoprocessor.
33. The implantable controller according to any one of the preceding aspects, wherein the wireless transceiver is configured to receive communication from a handheld external device.
34. The implantable controller according to any one of the preceding aspects, wherein the at least one instruction to the implantable medical device comprises an instruction for changing an operational state of the implantable medical device.
35. The implantable controller according to any one of the preceding aspects, wherein the wireless transceiver is configured to communicate wirelessly with the external device using electromagnetic waves at a frequency below 100 kHz.
36. The implantable controller according to aspect 35, wherein the wireless transceiver is configured to communicate wirelessly with the external device using electromagnetic waves at a frequency below 40 kHz.
37. The implantable controller according to any one of the preceding aspects, wherein: the wireless transceiver is configured to communicate wirelessly with the external device using a first communication protocol, the central unit is configured to communicate with the security module using a second communication protocol, and the first and second communication protocols are different.
38. The implantable controller according to any one of the preceding aspects, wherein the wireless transceiver is configured to communicate wirelessly with the external device using a standard network protocol.
39. The implantable controller according to aspect 38, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
40. The implantable controller according to any one of aspects 1-37, wherein the wireless transceiver is configured to communicate wirelessly with the external device using a proprietary network protocol.
41. A medical system comprising the implantable controller according to any one of the preceding aspects and an implantable medical device.
42. The medical system according to aspect 41, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable
engagement member configured to engage at least the second member such that the eccentric rotation of the engagement member causes the second member to pivot in relation to the main portion for displacing the second member relative to the first member for stretching the stomach wall between the first and second portions.
43. The medical system according to aspect 41, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first
and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least
partially enclosed by a flexible wall portion, and
the flexible wall portion causes the second member to bend or
pivot when hydraulic fluid is moved to or from the fluid chamber for stretching the stomach wall between the first and second portions.
44. The medical system according to aspect 41, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion, at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
45. The medical system according to aspect 41, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
46. The medical system according to aspect 41, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to: receive a signal from the acoustic sensor, and control the operation device on the basis of the signal received from the acoustic sensor.
47. The medical system according to aspect 41, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
48. The medical system according to aspect 41, wherein the implantable medical device comprises a medical device for actively stretching a stomach wall of a patient for creating a sensation of satiety, the medical device being implantable and comprising: a first member comprising:
   a first stomach engager configured to engage a first portion of the
stomach wall, and a second stomach engager portion configured to engage a second
portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion, an operation device comprising an actuator, wherein the
   operation device is configured to displace the actuator, such that the actuator is configured to: in a first state, operate the first member for displacing the first stomach engager of the first member in relation to the second stomach engager of the
   first member, for stretching the stomach wall, and in a second state, operate the second member for displacing the first stomach engager of the second member in relation to the second stomach engager of the second member, for stretching the stomach wall.
49. The medical system according to aspect 41, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
50. The medical system according to aspect 41, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
51. The medical system according to aspect 41, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
52. The medical system according to aspect 41, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
53. The medical system according to aspect 41, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
54. The medical system according to aspect 41, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall,
a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
55. The medical system according to aspect 41, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and move an operable third member in a third direction for
stretching a portion of the stomach wall between the third and second members, and
   a mechanical switching device configured to switch the operation device: from operating the first member to move in the first direction to operating the first member to move in the second direction, or
   from operating the first member to move in the first direction to operating the third member to move in the third direction.
56. The medical system according to aspect 41, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and in a second, a linear force is exerted in the shaft.
57. The medical system according to aspect 41, wherein the implantable medical device comprises an apparatus for treating obesity of a patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   58. The medical system according to aspect 41, wherein the implantable medical device comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 410a; 410b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
59. The medical system according to aspect 41, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 417; 418; 452) for operating the stretching device, when implanted, to stretch the stomach wall portion such that satiety is created,
   characterized in that the operable stretching device is adapted to be placed against the outside of the stomach wall.
60. The medical system according to aspect 41, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and the second engaging part is adapted to be engaged to a second area of the stomach wall, and wherein
   the stretching device is adapted to stretch a portion of the stomach wall between the first area and the second area.
61. The medical system according to aspect 41, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 432SE—eHealth_Variable_Impedance_1
1. An implantable medical device comprising a receiving unit comprising:
   at least one coil configured for receiving transcutaneously transferred energy,
   a measurement unit configured to measure a parameter related to the energy received by the coil,
   a variable impedance electrically connected to the coil,
   a switch placed between the variable impedance and the coil for switching off the electrical connection between the variable impedance and the coil, and
   a controller configured to:
   control the variable impedance for varying the impedance and thereby tune the coil based on the measured parameter, and
   control the switch for switching off the electrical connection between the variable impedance and the coil in response to the measured parameter exceeding a threshold value.
2. The implantable medical device according to aspect 1, wherein the controller is configured to vary the variable impedance in response to the measured parameter exceeding a threshold value.
3. The implantable medical device according to any one of aspects 1 and 2, wherein the measurement unit is configured to measure a parameter related to the energy received by the coil over a time period.
4. The implantable medical device according to any one of the preceding aspects, wherein the measurement unit is configured to measure a parameter related to a change in energy received by the coil.
5. The implantable medical device according to any one of the preceding aspects, wherein the first switch is placed at a first end portion of the coil, and wherein the implantable medical device further comprises a second switch placed at a second end portion of the coil, such that the coil can be completely disconnected from other portions of the implantable medical device.
6. The implantable medical device according to any one of the preceding aspects, wherein the receiving unit is configured to receive transcutaneously transferred energy in pulses according to a pulse pattern, and wherein the measurement unit is configured to measure a parameter related to the pulse pattern.
7. The implantable medical device according to aspect 6, wherein the controller is configured to control the variable impedance in response to the pulse pattern deviating from a predefined pulse pattern.
8. The implantable medical device according to aspect 6, wherein the controller is configured to control the switch for switching off the electrical connection between the variable impedance and the coil in response to the pulse pattern deviating from a predefined pulse pattern.
9. The implantable medical device according to any one of the preceding aspects, wherein the measurement unit is configured to measure a temperature in the implantable medical device or in the body of the patient, and wherein the controller is configured to control the first and second switch in response to the measured temperature.
10. The implantable medical device according to any one of the preceding aspects, wherein the variable impedance comprises a resistor and a capacitor.
11. The implantable medical device according to any one of the preceding aspects, wherein the variable impedance comprises a resistor and an inductor.
12. The implantable medical device according to any one of the preceding aspects, wherein the variable impedance comprises an inductor and a capacitor.
13. The implantable medical device according to any one of the preceding aspects, wherein the variable impedance comprises a digitally tuned capacitor.
14. The implantable medical device according to any one of the preceding aspects, wherein the variable impedance comprises a digital potentiometer.
15. The implantable medical device according to any one of the preceding aspects, wherein the variable impedance comprises a variable inductor.
16. The implantable medical device according to any one of the preceding aspects, wherein the variation of the impedance is configured to lower the active power that is received by the receiving unit.
17. The implantable medical device according to any one of the preceding aspects, wherein the variable impedance is placed in series with the coil.
18. The implantable medical device according to any one of aspects 1-16, wherein the variable impedance is placed parallel to the coil.
19. The implantable medical device according to any one of the preceding aspects, further comprising an energy storage unit connected to the receiving unit, and wherein the energy storage unit is configured for storing energy received by the receiving unit.
20. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of:
adapting the medical device to the curvature of the stomach of the patient, and facilitating insertion of the medical device into the body of the patient.
21. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
22. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
23. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
24. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and
   a force transferring element configured to transfer force hydraulically or mechanically from the remote unit to the main portion, for operating the second member.
25. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
26. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
27. The implantable medical device according to any one of the preceding aspects, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
28. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
29. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
30. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
31. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
32. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
33. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
34. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
35. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
36. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
37. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
38. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
39. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
40. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 433SE—eHealth_Variable_Impedance_2
1. An implantable medical device comprising a receiving unit comprising:
   at least one coil configured for receiving transcutaneously transferred energy, a measurement unit configured to measure a parameter related to the energy received by the coil,
   a first switch is placed at a first end portion of the coil,
   a second switch placed at a second end portion of the coil, such that the coil can be completely disconnected from other portions of the implantable medical device, and
   a controller configured to control the first and second switch for completely disconnecting the coil from other portions of the implantable medical device on the basis of the measured parameter.
2. The implantable medical device according to aspect 1, wherein the controller is configured to control the first and second switch in response to the measured parameter exceeding a threshold value.
3. The implantable medical device according to any one of aspects 1 and 2, wherein the measurement unit is configured to measure a parameter related to the energy received by the coil over a time period.
4. The implantable medical device according to any one of the preceding aspects, wherein the measurement unit is configured to measure a parameter related to a change in energy received by the coil.
5. The implantable medical device according to any one of the preceding aspects, wherein the receiving unit is configured to receive transcutaneously transferred energy in pulses according to a pulse pattern, and wherein the measurement unit is configured to measure a parameter related to the pulse pattern.
6. The implantable medical device according to aspect 5, wherein the controller is configured to control the first and second switch in response to the pulse pattern deviating from a predefined pulse pattern.
7. The implantable medical device according to any one of the preceding aspects, wherein the measurement unit is configured to measure a temperature in the implantable medical device or in the body of the patient, and wherein the controller is configured to control the first and second switch in response to the measured temperature.
8. The implantable medical device according to any one of the preceding aspects, further comprising an energy storage unit connected to the receiving unit, and wherein the energy storage unit is configured for storing energy received by the receiving unit.
9. The implantable medical device according to any one of the preceding aspects, further comprising an energy consuming part.
10. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of: adapting the medical device to the curvature of the stomach of the patient, and facilitating insertion of the medical device into the body of the patient.
11. The implantable medical device according to any one of the preceding aspects, wherein the implantable medical device comprises a medical device for actively stretching a stomach wall of a patient for creating a sensation of satiety, the medical device being implantable and comprising:
a first member comprising a first stomach engager configured to be fixated to a first portion of the stomach wall,
a second member comprising a second stomach engager portion configured to be fixated to a second portion of the stomach wall,
an operation device for operating the second member to displace the second member in a first direction relative to the first member, for stretching the stomach wall between the first and second portions, and
a main portion, wherein:
   the first and second members are connected to the main portion, and wherein at least the second member is pivotally connected to the main portion, such that the second member can be displaced in the first direction relative to the first member by the second member pivoting in relation to the main portion, and
   the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
12. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
13. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
14. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and
   a force transferring element configured to transfer force hydraulically or mechanically from the remote unit to the main portion, for operating the second member.
15. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
16. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
17. The implantable medical device according to any one of the preceding aspects, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
18. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
19. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
20. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
21. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
22. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and
   an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
23. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
24. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
25. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
26. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
27. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
28. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
29. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
30. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 434SE—eHealth_Variable_Impedance_3
1. An implantable medical device comprising a receiving unit comprising:
   at least one coil configured for receiving transcutaneously transferred energy, a measurement unit configured to measure a parameter related to the energy received by the coil, and
   a controller, wherein:
   the receiving unit is configured to receive transcutaneously transferred energy in pulses according to a pulse pattern, and
   the measurement unit is configured to measure a parameter related to the pulse pattern, and
   the controller is configured to control the receiving unit in response to the pulse pattern of the received energy deviating from a predetermined pulse pattern.
2. The implantable medical device according to aspect 1, further comprising at least one switch placed in series with the coil for switching of the coil, wherein the controller is configured to control the switch to switch of the coil in response to the pulse pattern of the received energy deviating from a predetermined pulse pattern.
3. The implantable medical device according to aspect 1, further comprising a variable impedance electrically connected to the coil, for varying the impedance and thereby tuning the coil, and wherein the controller is configured to control the variable impedance in response to the pulse pattern of the received energy deviating from a predetermined pulse pattern.
4. The implantable medical device according to any one of the preceding aspects, wherein the measurement unit is configured to measure a parameter related to the energy received by the coil over a time period.
5. The implantable medical device according to any one of the preceding aspects, wherein the measurement unit is configured to measure a parameter related to a change in energy received by the coil.
6. The implantable medical device according to any one of the preceding aspects, wherein the measurement unit is configured to measure a temperature in the implantable medical device or in the body of the patient, and wherein the controller is configured to control the first and second switch in response to the measured temperature.
7. The implantable medical device according to any one of the preceding aspects, wherein the first switch is placed at a first end portion of the coil, and wherein the implantable medical device further comprises a second switch placed at a second end portion of the coil, such that the coil can be completely disconnected from other portions of the implantable medical device.
8. The implantable medical device according to aspect 3, wherein the variable impedance comprises a resistor and a capacitor.
9. The implantable medical device according to aspect 3, wherein the variable impedance comprises a resistor and an inductor.
10. The implantable medical device according to aspect 3, wherein the variable impedance comprises an inductor and a capacitor.
11. The implantable medical device according to aspect 3, wherein the variable impedance comprises a digitally tuned capacitor.
12. The implantable medical device according to aspect 3, wherein the variable impedance comprises a digital potentiometer.
13. The implantable medical device according to aspect 3, wherein the variable impedance comprises a variable inductor.
14. The implantable medical device according to any one of aspects 3-12, wherein the variation of the impedance is configured to lower the active power that is received by the receiving unit.
15. The implantable medical device according to any one of aspects 3-13, wherein the variable impedance is placed in series with the coil.
16. The implantable medical device according to any one of aspects 3-13, wherein the variable impedance is placed parallel to the coil.
17. The implantable medical device according to any one of the preceding aspects, further comprising an energy storage unit connected to the receiving unit, and wherein the energy storage unit is configured for storing energy received by the receiving unit.
18. The implantable medical device according to any one of the preceding aspects, further comprising an energy consuming part.
19. The implantable medical device according to aspect 18, wherein the energy consuming part of the implantable medical device is configured to exert a force on a body portion of the patient.
20. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   first direction relative to the first member, for stretching the stomach wall between the first and second portion,
wherein at least one of the first and second member is flexible in a second direction substantially perpendicular to the first direction for at least one of: adapting the medical device to the curvature of the stomach of the patient, and facilitating insertion of the medical device into the body of the patient.
21. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
   a main portion, wherein:
      the first and second members are connected to the main portion, and wherein at least the second member is pivotally connected to the main portion, such that the second member can be displaced in the first direction relative to the first member by the second member pivoting in relation to the main portion, and
      the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
22. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
   a main portion, wherein:
      the first and second members are connected to the main portion, and wherein at least the second member is configured to bend or pivot in relation to the main portion, such that the second member can be displaced in the first direction relative to the first member by the second member bending or pivoting in relation to the main portion,
      at least the second member comprises a fluid chamber at least partially enclosed by a flexible wall portion, and
      the flexible wall portion causes the second member to bend or pivot when hydraulic fluid is moved to or from the fluid chamber for stretching the stomach wall between the first and second portions.
23. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
   a main portion, wherein:
      the first and second members are connected to the main portion,
      at least the second member is configured to bend or pivot in relation to the main portion, such that the second member can be displaced in a direction away from the first member by the second member bending or pivoting in relation to the main portion,
      the operation device comprises a first flexible cable for pulling on the second member for displacing the second member in the direction away from the first member, and
      the first flexible cable is fixated to the second member at the distal half of thereof.
24. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
   a main portion, wherein the first and second members are connected to the main portion,
   an operation device for operating at least the second member to displace the second member in a direction away from the first member, for stretching the stomach wall between the first and second portions, wherein a portion of the operation device is placed in a remote unit configured to be placed at a remote location in the body of the patient, and
   a force transferring element configured to transfer force hydraulically or mechanically from the remote unit to the main portion, for operating the second member.
25. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
      receive a signal from the acoustic sensor, and
      control the operation device on the basis of the signal received from the acoustic sensor.
26. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
      sense at least one parameter related to the patient swallowing, and
      be fixated to a structure of the body comprising bone, and
   a controller configured to:
      receive a signal from the sensor, and
      control the operation device on the basis of the signal received from the sensor.
27. The implantable medical device according to any one of the preceding aspects, wherein the implantable medical device comprises a medical device for actively stretching a stomach wall of a patient for creating a sensation of satiety, the medical device being implantable and comprising:
   a first member comprising:
      a first stomach engager configured to engage a first portion of the stomach wall, and
      a second stomach engager portion configured to engage a second portion of the stomach wall,
   a second member comprising:
      a first stomach engager configured to engage a third portion of the stomach wall, and
      a second stomach engager portion configured to engage a fourth portion of the stomach wall,
   a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
28. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
29. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
      a first and second pulley,
      a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
30. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient and comprising a closed curve,
   a first member configured to be fixated to the stomach wall of the patient within the footprint of the second member,
   wherein the first member is operably connected to the second member such that the first member can be displaced:
      relative to the second member, and
      within the footprint of the second member, for stretching a portion of the stomach wall of the patient between the second and first member.
31. The implantable medical device according to any one of the preceding aspects, wherein the implantable medical device comprises a medical device for actively stretching a stomach wall of a patient for creating a sensation of satiety, the medical device being implantable and comprising:
   a second member configured to be fixated to the stomach wall of the patient, and
   an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
32. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall, and
   a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
33. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
   a force transferring element configured to transfer mechanical force from the remote unit to the first member, for operating the first member.
34. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall,
   a second member configured to be fixated to a second portion of the stomach wall,
   an operation device configured to:
      move at least a portion of the first member in a first direction relative to at least one portion of the second member, for stretching a portion of the stomach wall between the first and second members, and
      at least one of:
         move at least a portion of the operable first member in a second direction relative to at least one portion of the second member, and
         move an operable third member in a third direction for stretching a portion of the stomach wall between the third and second members, and
   a mechanical switching device configured to switch the operation device:
      from operating the first member to move in the first direction to operating the first member to move in the second direction, or
      from operating the first member to move in the first direction to operating the third member to move in the third direction.
35. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is
   configured to switch the force exerted on shaft such that: in a first state, a rotational force is exerted on the shaft, and in a second, a linear force is exerted in the shaft.
36. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
37. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
38. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created, characterized in that the operable stretching device is adapted to be placed against the outside of the stomach wall.
39. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and the second engaging part is adapted to be engaged to a second area of the stomach wall, and wherein
   the stretching device is adapted to stretch a portion of the stomach wall between the first area and the second area.
40. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 377-Electro_Subcutaneous_Control_Pop-Rivet2_Bellows

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion, and
   a hermetic seal arrangement configured to enclose the connecting portion so as to prevent fluid from the patient to enter the connecting portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes, and
   the connecting portion comprises a flexible structure enabling the connecting portion to flex.
2. The implantable medical device according to aspect 1, wherein the flexible structure is configured to allow the connecting portion to flex in more than one direction.
3. The implantable medical device according to aspect 2, wherein the flexible structure is configured to allow the connecting portion to flex in all directions.
4. The implantable medical device according to any one of the preceding aspects, wherein the flexible structure comprises a bellows.
5. The implantable medical device according to aspect 4, wherein the bellows is a metallic bellows.
6. The implantable medical device according to aspect 5, wherein the metallic bellows is welded.
7. The implantable medical device according to any one of aspects 4 to 6, wherein the bellows is a titanium bellows.
8. The implantable medical device according to any one of aspects 4 to 8, wherein the bellows form part of the hermetic seal arrangement.
9. The implantable medical device according to any one of the preceding aspects, wherein the flexible structure comprises elevated and lowered portions enabling said flexing of the connecting portion.
10. The implantable medical device according to aspect 9, wherein the elevated and lowered portions are configured to enable the connecting portion to be compressed and/or expanded.
11. The implantable medical device according to any one of the preceding aspects, wherein the flexible structure has a substantially cylindrical shape.
12. The implantable medical device according to any one of the preceding aspects, wherein the flexible structure is configured to seal against the first portion and/or the second portion.
13. The implantable medical device according to any one of the preceding aspects, wherein the connecting portion and the second portion are hermetically sealed from the first portion.
14. The implantable medical device according to aspect 13, wherein the hermetic seal arrangement encloses the connecting portion and the second portion so as to hermetically seal the connecting portion and the second portion from the first portion.
15. The implantable medical device according to any one of the preceding aspects, wherein the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.
16. The implantable medical device according to aspect 15, wherein the first portion comprises a first energy storage unit connected to the first wireless energy receiver.
17. The implantable medical device according to any one of aspects 15 and 16, wherein the second portion comprises a second energy storage unit connected to the second wireless energy receiver.
18. The implantable medical device according to aspect 17, wherein at least one of the first and second energy storage unit is a solid-state battery.
19. The implantable medical device according to aspect 18, wherein the solid-state battery is a thionyl-chloride battery.
20. The implantable medical device according to any one of aspects 17-19, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.
21. The implantable medical device according to any one of aspects 15-20, wherein the first portion comprises a first controller comprising at least one processing unit.
22. The implantable medical device according to any one of aspects 15-21, wherein the second portion comprises a second controller comprising at least one processing unit.
23. The implantable medical device according to any one of aspects 21 and 22, wherein at least one of the first and second controller is connected to a wireless transceiver for communicating wirelessly with an external device.
24. The implantable medical device according to any one of aspects 21 and 22, wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
25. The implantable medical device according to aspect 24, wherein the second controller is connected to the second wireless communication receiver for receiving wireless communication from the first portion.
26. The implantable medical device according to any one of aspects 15-25, wherein the first wireless energy receiver comprises a first coil and the wireless energy transmitter comprises a second coil.
27. The implantable medical device according to any one of aspects 15-26, wherein the first portion comprises a combined coil, wherein the combined coil is configured to receive energy wirelessly from an external wireless energy transmitter, and transmit energy wirelessly to the second wireless receiver of the second portion.
28. The implantable medical device according to any one of aspects 26 and 27, wherein at least one of the coils are embedded in a ceramic material.
29. The implantable medical device according to any one of the preceding aspects, further comprising a housing configured to enclose at least the first portion, and wherein a first portion of the housing is made from titanium and a second portion of the housing is made from a ceramic material.
30. The implantable medical device according to any one of the preceding aspects, wherein the implantable medical device comprises a medical device for actively stretching a stomach wall of a patient for creating a sensation of satiety, the medical device being implantable and comprising: a first member configured to be fixated to a first portion of the stomach wall, a second member configured to be fixated to a second portion of the stomach wall, an operation device for operating the second member to displace the second member in a first direction relative to the first member, for stretching the stomach wall between the first and second portion, wherein at least one of the first and second member is flexible in a second direction substantially perpendicular to the first direction for at least one of: adapting the medical device to the curvature of the stomach of the patient, and facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable
engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion, at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received
from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the
stomach wall, and
a second stomach engager portion configured to engage a second
portion of the stomach wall,
   a second member comprising:
a first stomach engager configured to engage a third portion of the
stomach wall, and
a second stomach engager portion configured to engage a fourth
portion of the stomach wall,
a main portion, wherein the first and second members are operably connected to
the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises: a first and second pulley, a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member,
for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and at least one of:
move at least a portion of the operable first member in a second direction relative to at least one portion of the second member, and move an operable third member in a third direction for
stretching a portion of the stomach wall between the third and second members, and a mechanical switching device configured to switch the operation device:
from operating the first member to move in the first direction to operating the first member to move in the second direction, or
from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
an electrical machine for transforming electrical energy to mechanical force, a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
47. The implantable medical device according to any one of the preceding aspects, wherein the implantable medical device comprises a device for treating obesity of a patient comprising:
at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 434_Electro_Subcutaneous_Control_Pop-Rivet2_Decreasing-Area

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   wherein the connecting portion and the second portion are configured to form a unit having a central axis extending from a first end of said unit to a second end of said unit, the first end being proximal to the first portion and the second end being distal to the first portion, wherein a physical footprint of said unit perpendicular to the central axis decreases continuously or stepwise from the first end to the second end of said unit.
2. The implantable medical device according to aspect 1, wherein said physical footprint comprises a cross-sectional area perpendicular to the central axis.
3. The implantable medical device according to aspect 1 or 2, wherein the connecting portion and the second portion are one of:
   configured to reversibly connect to each other to form said unit; or
   configured to irreversibly connect to each other to form said unit; or
   configured as a single body forming said unit.
4. The implantable medical device according to any one of the preceding aspects, wherein said unit comprises an angled section forming a bend in said unit.
5. The implantable medical device according to aspect 4, wherein the bend is between 150 and 165°, such as between 300 and 150°, such as between 450 and 135°, such as substantially 90°.
6. The implantable medical device according to any one of the preceding aspects, wherein
   the first portion comprises a first wireless energy receiver for receiving energy transmitted wirelessly by an external wireless energy transmitter, and an internal wireless energy transmitter configured to transmit energy wirelessly to the second portion, and
   the second portion comprises a second wireless energy receiver configured to receive energy transmitted wirelessly by the internal wireless energy transmitter.
7. The implantable medical device according to aspect 6, wherein the first portion comprises a first energy storage unit connected to the first wireless energy receiver.
8. The implantable medical device according to any one of aspects 6 and 7, wherein the second portion comprises a second energy storage unit connected to the second wireless energy receiver.
9. The implantable medical device according to aspect 8, wherein at least one of the first and second energy storage unit is a solid-state battery.
10. The implantable medical device according to aspect 9, wherein the solid-state battery is a thionyl-chloride battery.
11. The implantable medical device according to any one of aspects 8-10, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
12. The implantable medical device according to any one of aspects 6-11, wherein the first portion comprises a first controller comprising at least one processing unit.
13. The implantable medical device according to any one of aspects 6-12, wherein the second portion comprises a second controller comprising at least one processing unit.
14. The implantable medical device according to any one of aspects 12 and 13, wherein at least one of the first and second controller is connected to a wireless transceiver for communicating wirelessly with an external device.
15. The implantable medical device according to any one of aspects 12 and 13, wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
16. The implantable medical device according to aspect 15, wherein the second controller is connected to the second wireless communication receiver for receiving wireless communication from the first portion.
17. The implantable medical device according to any one of aspects 6-16, wherein the first wireless energy receiver comprises a first coil and the wireless energy transmitter comprises a second coil.
18. The implantable medical device according to any one of aspects 6-17, wherein the first portion comprises a combined coil, wherein the combined coil is configured to receive energy wirelessly from an external wireless energy transmitter, and transmit energy wirelessly to the second wireless receiver of the second portion.
19. The implantable medical device according to any one of aspects 17 and 18, wherein at least one of the coils are embedded in a ceramic material.
20. The implantable medical device according to any one of the preceding aspects, further comprising a housing configured to enclose at least the first portion, and wherein a first portion of the housing is made from titanium and a second portion of the housing is made from a ceramic material.
21. The implantable medical device according to aspect 20, wherein the portion of the housing made from a ceramic material comprises at least one coil embedded in the ceramic material.
22. The implantable medical device according to any one of the preceding aspects, further comprising a housing configured to enclose at least the second portion, and wherein a first portion of the housing is made from titanium and a second portion of the housing is made from a ceramic material.
23. The implantable medical device according to aspect 22, wherein the portion of the housing made from a ceramic material comprises at least one coil embedded in the ceramic material.
24. The implantable medical device according to any one of the preceding aspects, wherein the first portion is detachably connected to at least one of the second portion and the connecting portion.
25. The implantable medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
26. The implantable medical device according to any one of the preceding aspects, wherein a connecting interface between the connecting portion and the second portion is excentric with respect to the second portion.
27. The implantable medical device according to any one of the preceding aspects, wherein a connecting interface between the connecting portion and the first portion is excentric with respect to the first portion.
28. The implantable medical device according to any one of the preceding aspects, wherein the second portion has a first end and a second end opposing the first end, wherein the second portion has a length between the first and second end.
29. The implantable medical device according to aspect 28, wherein the first end and second end are separated in a direction parallel to the second plane.
30. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of:
adapting the medical device to the curvature of the stomach of the patient, and
facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable
engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion, at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion, an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received
from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second
portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member,
for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and move an operable third member in a third direction for stretching a portion of the stomach wall between the third and second members, and
   a mechanical switching device configured to switch the operation device:
from operating the first member to move in the first direction to operating the first member to move in the second direction, or
from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   47. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with

### Aspect 435_Electro_Subcutaneous_Control_Pop-Rivet2_Electric-Motor-Orientation

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion, and
   an electric motor,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   at least part of the electric motor is arranged within the connecting portion.
2. The implantable medical device according to aspect 1, wherein the electric motor is arranged within the connecting portion within an imaginary boundary defined by the first surface of the first portion extending through the connecting portion.
3. The implantable medical device according to aspect 1 or 2, wherein the electric motor is arranged within the connecting portion within an imaginary boundary defined by the second surface of the second portion extending through the connecting portion.
4. The implantable medical device according to aspect 1, wherein the electric motor is fully arranged in the connecting portion within imaginary boundaries defined by the first surface of the first portion extending through the connecting portion and the second surface of the second portion extending through the connecting portion respectively.
3. The implantable medical device according to aspect 1 or 2, wherein the electric motor is arranged such that its longest dimension extends in a direction substantially perpendicular to the first, second and third cross-sectional areas.
4. The implantable medical device according to any one of the preceding aspects, wherein the electric motor is arranged such that its longest dimension extends in a direction between the first portion and the second portion.
5. The implantable medical device according to aspect 5, wherein the worm drive is configured to transfer mechanical force from the electric motor to an implantable body engaging portion being external to the implantable medical device.
6. The implantable medical device according to any one of the preceding aspects, wherein the electric motor extends through the connecting portion into the first portion and/or the second portion.
7. The implantable medical device according to aspect 6, wherein the electric motor extends through an imaginary boundary defined by the first surface of the first portion extending through the connecting portion.
8. The implantable medical device according to aspect 6, wherein the electric motor extends through an imaginary boundary defined by the second surface of the second portion extending through the connecting portion.
9. The implantable medical device according to aspect 6, wherein the electric motor extends through imaginary boundaries defined by the first surface of the first portion extending through the connecting portion and the second surface of the second portion extending through the connecting portion respectively.
10. The implantable medical device according to any one of the preceding aspects, further comprising a gear arrangement operatively connected to the electric motor wherein the gear arrangement is partly or fully arranged in one of the first portion and the second portion.
11. The implantable medical device according to aspect 10, wherein the gear arrangement is arranged within the connecting portion within an imaginary boundary defined by the first surface of the first portion extending through the connecting portion.
12. The implantable medical device according to aspect 10 or 11, wherein the gear arrangement is arranged within the connecting portion within an imaginary boundary defined by the second surface of the second portion extending through the connecting portion.
13. The implantable medical device according to any one of aspects 10 to 12, wherein the gear arrangement is fully arranged in the connecting portion within imaginary boundaries defined by the first surface of the first portion extending through the connecting portion and the second surface of the second portion extending through the connecting portion respectively.
14. The implantable medical device according to any one of aspects 10 to 13, wherein the gear arrangement extends through the connecting portion into the first portion and/or the second portion.
15. The implantable medical device according to aspect 14, wherein the gear arrangement extends through an imaginary boundary defined by the first surface of the first portion extending through the connecting portion.
16. The implantable medical device according to aspect 14, wherein the gear arrangement extends through an imaginary boundary defined by the second surface of the second portion extending through the connecting portion.
17. The implantable medical device according to aspect 14, wherein the gear arrangement extends through imaginary boundaries defined by the first surface of the first portion extending through the connecting portion and the second surface of the second portion extending through the connecting portion respectively.
18. The implantable medical device according to any one of aspects 10 to 17, wherein the gear arrangement is configured to transfer mechanical force from the electric motor to an implantable body engaging portion being external to the implantable medical device.
19. The implantable medical device according to any one of aspects 10 to 18, wherein the gear arrangement is a worm drive or comprises a worm drive.
20. The implantable medical device according to any one of the preceding aspects, wherein the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.
21. The implantable medical device according to aspect 20, wherein the first portion comprises a first energy storage unit connected to the first wireless energy receiver.
22. The implantable medical device according to any one of aspects 20 and 21, wherein the second portion comprises a second energy storage unit connected to the second wireless energy receiver.
23. The implantable medical device according to aspect 22, wherein at least one of the first and second energy storage unit is a solid-state battery.
24. The implantable medical device according to aspect 23, wherein the solid-state battery is a thionyl-chloride battery.
25. The implantable medical device according to any one of aspects 22-24, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.
26. The implantable medical device according to any one of aspects 20-25, wherein the first portion comprises a first controller comprising at least one processing unit.
27. The implantable medical device according to any one of aspects 20-26, wherein the second portion comprises a second controller comprising at least one processing unit.
28. The implantable medical device according to any one of aspects 26 and 27, wherein at least one of the first and second controller is connected to a wireless transceiver for communicating wirelessly with an external device.
29. The implantable medical device according to any one of aspects 26 and 27, wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
30. The implantable medical device according to any one of the preceding aspects, wherein the implantable medical device comprises a medical device for actively stretching a stomach wall of a patient for creating a sensation of satiety, the medical device being implantable and comprising: a first member configured to be fixated to a first portion of the stomach wall, a second member configured to be fixated to a second portion of the stomach wall, first direction relative to the first member, for stretching the stomach wall between the first and second portion, wherein at least one of the first and second member is flexible in a second direction substantially perpendicular to the first direction for at least one of: adapting the medical device to the curvature of the stomach of the patient, and facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion, and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and a main portion, wherein:
the first and second members are connected to the main portion, and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and
a main portion, wherein:
the first and second members are connected to the main portion, at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the
stomach wall, and
a second stomach engager portion configured to engage a fourth
portion of the stomach wall,
a main portion, wherein the first and second members are operably connected to
the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and within the footprint of the second member, for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a
second direction relative to at least one portion of the second member, and
move an operable third member in a third direction for stretching a portion of the stomach wall between the third and second members, and a mechanical switching device configured to switch the operation device:
from operating the first member to move in the first direction to operating the first member to move in the second direction, or
from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
an electrical machine for transforming electrical energy to mechanical force,
a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   47. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created, characterized in that the operable stretching device is adapted to be placed against the outside of the stomach wall.
   49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with

### Aspect 436_Electro_Subcutaneous_Control_Pop-Rivet2_First-Portion

1. An implantable medical device configured to be held in position by a tissue portion of a patient, the medical device comprising:
   a first portion configured to be placed on a first side of the tissue portion, the first portion comprising a first surface configured to face a first tissue surface of the first side of the tissue portion, the first portion being further configured to connect, directly or indirectly, to a second portion placed on a second side of the tissue portion opposing the first side,
   wherein the first portion comprises an internal wireless energy transmitter configured to transmit energy wirelessly to the second portion.
2. The implantable medical device according to aspect 1, wherein the first portion is configured to connect, directly or indirectly, to the second portion, via a connecting portion configured to extend through a hole in the tissue portion, the hole extending between the first side of the tissue portion and the second side of the tissue portion.
3. The implantable medical device according to aspect 2, further comprising the connecting portion.
4. The implantable medical device according to aspect 3, wherein the connecting portion is integrally formed with the first portion.
5. The implantable medical device according to aspect 3, wherein the connecting portion is a separate component with regard to the first portion, the connecting portion being configured to be connected to the first portion.
6. The implantable medical device according to any one of aspects 2-5, wherein the first portion has a first cross-sectional area in a first plane and the connecting portion has a second cross-sectional area in a second plane, wherein the first and second planes are parallel to each other, wherein the second cross-sectional area is smaller than the first cross-sectional area, such that the first portion and the second portion are prevented from travelling through the hole in the tissue portion in a direction perpendicular to the first and second planes.
7. The implantable medical device according to any one of the preceding aspects, wherein the first portion is configured to detachably connect, directly or indirectly, to the second portion.
8. The implantable medical device according to any one of the preceding aspects, wherein the first portion comprises a first wireless energy receiver for receiving energy transmitted wirelessly by an external wireless energy transmitter.
9. The implantable medical device according to aspect 8, wherein the first portion comprises a first energy storage unit connected to the first wireless energy receiver.
10. The implantable medical device according to aspect 9, wherein the first energy storage unit is a solid-state battery.
11. The implantable medical device according to aspect 10, wherein the solid-state battery is a thionyl-chloride battery.
12. The implantable medical device according to any one of aspects 8-11, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to a second wireless energy receiver in the second portion.
13. The implantable medical device according to any one of the preceding aspects, wherein the first portion comprises a first controller comprising at least one processing unit.
14. The implantable medical device according to aspect 13, wherein the first controller is connected to a wireless transceiver for communicating wirelessly with an external device.
15. The implantable medical device according to aspect 13, wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
16. The implantable medical device according to any one of aspects 8-15, wherein the first wireless energy receiver comprises a first coil and the wireless energy transmitter comprises a second coil.
17. The implantable medical device according to any one of aspects 8-16, wherein the first portion comprises a combined coil, wherein the combined coil is configured to receive energy wirelessly from an external wireless energy transmitter, and transmit energy wirelessly to the second wireless receiver of the second portion.
18. The implantable medical device according to any one of aspects 16 and 17, wherein at least one of the coils are embedded in a ceramic material.
19. The implantable medical device according to aspect 3, wherein the connecting portion comprises a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
20. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of:
adapting the medical device to the curvature of the stomach of the patient, and facilitating insertion of the medical device into the body of the patient.
21. The implantable medical device according to any one of the preceding aspects, wherein the implantable medical device comprises a medical device for actively stretching a stomach wall of a patient for creating a sensation of satiety, the medical device being implantable and comprising: a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable
engagement member configured to engage at least the second member such that the eccentric rotation of the engagement member causes the second member to pivot in relation to the main portion for displacing the second member relative to the first member for stretching the stomach wall between the first and second portions.
22. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least
partially enclosed by a flexible wall portion, and
the flexible wall portion causes the second member to bend or pivot when hydraulic fluid is moved to or from the fluid chamber for stretching the stomach wall between the first and second portions.
23. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
24. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
25. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
26. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
27. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the
stomach wall, and
a second stomach engager portion configured to engage a second
portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
28. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
29. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
30. The implantable medical device according to any one of the preceding aspects, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
31. The implantable medical device according to any one of the preceding aspects, wherein the implantable medical device comprises a medical device for actively stretching a stomach wall of a patient for creating a sensation of satiety, the medical device being implantable and comprising:
a second member configured to be fixated to the stomach wall of the patient, and an operable first member connected to the second member and configured to be fixated to the stomach wall at least partially within the footprint of the second member, wherein
the first member is configured to be operated to deform such that a first portion of the first member is moved away from a first portion of the second member, for stretching a portion of the stomach wall between the first portion of the first member and the first portion of the second member.
32. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
33. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall,
a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
34. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and move an operable third member in a third direction for stretching a portion of the stomach wall between the third and second members, and
   a mechanical switching device configured to switch the operation device: from operating the first member to move in the first direction to operating the first member to move in the second direction, or
from operating the first member to move in the first direction to operating the third member to move in the third direction.
35. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
36. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
37. The implantable medical device according to any one of the preceding aspects, wherein the implantable medical device comprises a device for treating obesity of a patient comprising:
at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and the second part is adapted to be engaged to a second area of the stomach wall, and wherein the stretching device is adapted to stretch a portion of the stomach wall between the first area and the second area.
   38. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
39. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
40. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 438_Electro_Subcutaneous_Control_Pop-Rivet2_Same-Shape-A

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   the first portion is configured to be placed subcutaneously in the patient, and wherein the first portion comprises a connecting interface arrangement for transferring wired energy and/or wired communication signals and/or fluid to an additional implant in the patient.
2. The implantable medical device according to aspect 1, wherein a height of the first portion measured in a plane perpendicular to the first plane is 15 mm or less, such as 10 mm or less, such as 7 mm or less, such as 5 mm or less.
3. The implantable medical device according to aspect 1 or 2, wherein the connecting interface arrangement comprises a port for transferring fluid from the first portion to said additional implant.
4. The implantable medical device according to aspect 3, further comprising at least one conduit or tube for transferring said fluid, wherein the at least one conduit or tube is connected to the port.
5. The implantable medical device according to any one of the preceding aspects, further comprising at least one wire for energy and/or communication signals connected to the connecting interface arrangement.
6. The implantable medical device according to aspect 2, wherein the height of the first portion is a maximum height.
7. The implantable medical device according to any one of the preceding aspects, wherein
   the first portion comprises a first wireless energy receiver for receiving energy transmitted wirelessly by an external wireless energy transmitter, and an internal wireless energy transmitter configured to transmit energy wirelessly to the second portion, and
   the second portion comprises a second wireless energy receiver configured to receive energy transmitted wirelessly by the internal wireless energy transmitter.
8. The implantable medical device according to aspect 7, wherein the first portion comprises a first energy storage unit connected to the first wireless energy receiver.
9. The implantable medical device according to any one of aspects 7 and 8, wherein the second portion comprises a second energy storage unit connected to the second wireless energy receiver.
10. The implantable medical device according to aspect 9, wherein at least one of the first and second energy storage unit is a solid-state battery.
11. The implantable medical device according to aspect 10, wherein the solid-state battery is a thionyl-chloride battery.
12. The implantable medical device according to any one of aspects 9-11, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.
13. The implantable medical device according to any one of aspects 7-12, wherein the first portion comprises a first controller comprising at least one processing unit.
14. The implantable medical device according to any one of aspects 7-13, wherein the second portion comprises a second controller comprising at least one processing unit.
15. The implantable medical device according to any one of aspects 13 and 14, wherein at least one of the first and second controller is connected to a wireless transceiver for communicating wirelessly with an external device.
16. The implantable medical device according to any one of aspects 13 and 14, wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
17. The implantable medical device according to aspect 16, wherein the second controller is connected to the second wireless communication receiver for receiving wireless communication from the first portion.
18. The implantable medical device according to any one of aspects 7-17, wherein the first wireless energy receiver comprises a first coil and the wireless energy transmitter comprises a second coil.
19. The implantable medical device according to any one of aspects 7-18, wherein the first portion comprises a combined coil, wherein the combined coil is configured to receive energy wirelessly from an external wireless energy transmitter, and transmit energy wirelessly to the second wireless receiver of the second portion.
20. The implantable medical device according to any one of aspects 18 and 19, wherein at least one of the coils are embedded in a ceramic material.
21. The implantable medical device according to any one of the preceding aspects, further comprising a housing configured to enclose at least the first portion, and wherein a first portion of the housing is made from titanium and a second portion of the housing is made from a ceramic material.
22. The implantable medical device according to aspect 21, wherein the portion of the housing made from a ceramic material comprises at least one coil embedded in the ceramic material.
23. The implantable medical device according to any one of the preceding aspects, further comprising a housing configured to enclose at least the second portion, and wherein a first portion of the housing is made from titanium and a second portion of the housing is made from a ceramic material.
24. The implantable medical device according to aspect 23, wherein the portion of the housing made from a ceramic material comprises at least one coil embedded in the ceramic material.
25. The implantable medical device according to any one of the preceding aspects, wherein the first portion is detachably connected to at least one of the second portion and the connecting portion.
26. The implantable medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
27. The implantable medical device according to any one of the preceding aspects, wherein a connecting interface between the connecting portion and the second portion is excentric with respect to the second portion.
28. The implantable medical device according to any one of the preceding aspects, wherein a connecting interface between the connecting portion and the first portion is excentric with respect to the first portion.
29. The implantable medical device according to any one of the preceding aspects, wherein the second portion has a first end and a second end opposing the first end, wherein the second portion has a length between the first and second end.
30. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a
first direction relative to the first member, for stretching the stomach wall between the first and second portion,
wherein at least one of the first and second member is flexible in a second direction substantially perpendicular to the first direction for at least one of:
adapting the medical device to the curvature of the stomach of the patient, and facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least
partially enclosed by a flexible wall portion, and
the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall, a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and be fixated to a structure of the body comprising bone, and
a controller configured to:
receive a signal from the sensor, and
control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and
at least one of:
move at least a portion of the operable first member in a second direction relative to at least one portion of the second member, and
move an operable third member in a third direction for stretching a portion of the stomach wall between the third and second members, and a mechanical switching device configured to switch the operation device:
from operating the first member to move in the first direction to operating the first member to move in the second direction, or
from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   47. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 438_Electro_Subcutaneous_Control_Pop-Rivet2_Same-Shape-B

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes,
   the first portion and the second portion are configured to be placed subcutaneously in the patient, such that the implantable medical device can be placed with either of the first portion and the second portion on the first side of the tissue portion.
2. The implantable medical device according to aspect 1, wherein a height of the second portion measured in a plane perpendicular to the second plane is 15 mm or less, such as 10 mm or less, such as 7 mm or less, such as 5 mm or less.
3. The implantable medical device according to aspect 1 or 2, wherein the first portion has a length in a plane parallel to the first plane, wherein the second portion has a length in a plane parallel to the second plane, and wherein the length of the first portion differ no more than 30% with regard to the length of the second portion, such as wherein the length of the first portion differ no more than 15% with regard to the length of the second portion, such as wherein the length of the first portion differ no more than 5% with regard to the length of the second portion, such as wherein the length of the first portion differ no more than 1% with regard to the length of the second portion.
4. The implantable medical device according to any one of the preceding aspects, wherein the first portion has a width in a plane parallel to the first plane, wherein the second portion has a width in a plane parallel to the second plane, and wherein the width of the first portion differ no more than 30% with regard to the width of the second portion, such as wherein the width of the first portion differ no more than 15% with regard to the width of the second portion, such as wherein the width of the first portion differ no more than 5% with regard to the width of the second portion, such as wherein the width of the first portion differ no more than 1% with regard to the width of the second portion.
5. The implantable medical device according to any one of the preceding aspects, wherein the first portion has a height in a plane perpendicular to the first plane, and wherein the height of the first portion differ no more than 30% with regard to the height of the second portion, such as wherein the height of the first portion differ no more than 15% with regard to the height of the second portion, such as wherein the height of the first portion differ no more than 5% with regard to the height of the second portion, such as wherein the height of the first portion differ no more than 1% with regard to the height of the second portion.
6. The implantable medical device according to any one of the preceding aspects, wherein a height of the first portion measured in a plane perpendicular to the first plane is 15 mm or less, such as 10 mm or less, such as 7 mm or less, such as 5 mm or less.
7. The implantable medical device according to any one of the preceding aspects, wherein
   the first portion comprises a first wireless energy receiver for receiving energy transmitted wirelessly by an external wireless energy transmitter, and an internal wireless energy transmitter configured to transmit energy wirelessly to the second portion, and
   the second portion comprises a second wireless energy receiver configured to receive energy transmitted wirelessly by the internal wireless energy transmitter.
8. The implantable medical device according to aspect 7, wherein the first portion comprises a first energy storage unit connected to the first wireless energy receiver.
9. The implantable medical device according to any one of aspects 7 and 8, wherein the second portion comprises a second energy storage unit connected to the second wireless energy receiver.
10. The implantable medical device according to aspect 9, wherein at least one of the first and second energy storage unit is a solid-state battery.
11. The implantable medical device according to aspect 10, wherein the solid-state battery is a thionyl-chloride battery.
12. The implantable medical device according to any one of aspects 9-11, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.
13. The implantable medical device according to any one of aspects 7-12, wherein the first portion comprises a first controller comprising at least one processing unit.
14. The implantable medical device according to any one of aspects 7-13, wherein the second portion comprises a second controller comprising at least one processing unit.
15. The implantable medical device according to any one of aspects 13 and 14, wherein at least one of the first and second controller is connected to a wireless transceiver for communicating wirelessly with an external device.
16. The implantable medical device according to any one of aspects 13 and 14, wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
17. The implantable medical device according to aspect 16, wherein the second controller is connected to the second wireless communication receiver for receiving wireless communication from the first portion.
18. The implantable medical device according to any one of aspects 7-17, wherein the first wireless energy receiver comprises a first coil and the wireless energy transmitter comprises a second coil.
19. The implantable medical device according to any one of aspects 7-18, wherein the first portion comprises a combined coil, wherein the combined coil is configured to receive energy wirelessly from an external wireless energy transmitter, and transmit energy wirelessly to the second wireless receiver of the second portion.
20. The implantable medical device according to any one of aspects 18 and 19, wherein at least one of the coils are embedded in a ceramic material.
21. The implantable medical device according to any one of the preceding aspects, further comprising a housing configured to enclose at least the first portion, and wherein a first portion of the housing is made from titanium and a second portion of the housing is made from a ceramic material.
22. The implantable medical device according to aspect 21, wherein the portion of the housing made from a ceramic material comprises at least one coil embedded in the ceramic material.
23. The implantable medical device according to any one of the preceding aspects, further comprising a housing configured to enclose at least the second portion, and wherein a first portion of the housing is made from titanium and a second portion of the housing is made from a ceramic material.
24. The implantable medical device according to aspect 23, wherein the portion of the housing made from a ceramic material comprises at least one coil embedded in the ceramic material.
25. The implantable medical device according to any one of the preceding aspects, wherein the first portion is detachably connected to at least one of the second portion and the connecting portion.
26. The implantable medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
27. The implantable medical device according to any one of the preceding aspects, wherein a connecting interface between the connecting portion and the second portion is excentric with respect to the second portion.
28. The implantable medical device according to any one of the preceding aspects, wherein a connecting interface between the connecting portion and the first portion is excentric with respect to the first portion.
29. The implantable medical device according to any one of the preceding aspects, wherein the second portion has a first end and a second end opposing the first end, wherein the second portion has a length between the first and second end.
30. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of:
adapting the medical device to the curvature of the stomach of the patient, and
facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion,
and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and the flexible wall portion causes the second member to bend or
pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to: receive a signal from the acoustic sensor, and
control the operation device on the basis of the signal received from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and
be fixated to a structure of the body comprising bone, and
a controller configured to:
receive a signal from the sensor, and
control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising:
a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member,
for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and
a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and
second members, and
at least one of:
move at least a portion of the operable first member in a second direction relative to at least one portion of the second member, and move an operable third member in a third direction for stretching a portion of the stomach wall between the third and second members, and
   a mechanical switching device configured to switch the operation device: from operating the first member to move in the first direction to operating the first member to move in the second direction, or
from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force, a transmission for transforming rotational force created by the electrical machine to a linear force,
   a shaft for transferring rotational and linear force from the operation device to the body engaging portion of the medical implant,
   a switch placed between the electrical machine and the shaft, wherein the switch is configured to switch the force exerted on shaft such that:
   in a first state, a rotational force is exerted on the shaft, and
   in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
47. The implantable medical device according to any one of the preceding aspects, wherein the implantable medical device comprises a device for treating obesity of a patient comprising:
at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with

### Aspect 439_Electro_Subcutaneous_Control_Pop-Rivet2_First-Portion-Polymer

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   the second portion is hermetically sealed by means of an outer wall of the second portion comprising titanium.
2. The implantable medical device according to aspect 1, wherein the first portion comprises an outer wall comprising a polymer material.
3. The implantable medical device according to aspect 2, wherein the outer wall of the first portion consists of the polymer material.
4. The implantable medical device according to any one of the preceding aspects, wherein the second portion is hermetically sealed with respect to the connecting portion and the first portion.
5. The implantable medical device according to any one of the preceding aspects, wherein the outer wall of the second portion comprises a ceramic portion integrated in, or brazed to, the titanium.
6. The implantable medical device according to aspect 5, wherein the ceramic portion of the second portion comprises at least one metallic lead travelling through the ceramic portion for transferring electrical energy or information from within the second portion to an outside of the second portion and/or from the outside of the second portion to an inside of the second portion.
7. The implantable medical device according to aspect 5 or 6, wherein the at least one metallic lead is integrated in, or brazed to, the ceramic portion of the second portion, such that the at least one metallic lead can pass said ceramic portion without being further insulated.
8. The implantable medical device according to any one of aspects 5 to 7, wherein the connecting portion comprises an outer wall comprising titanium.
9. The implantable medical device according to aspect 8, wherein the outer wall of the connecting portion comprises a ceramic portion integrated in, or brazed to, the titanium.
10. The implantable medical device according to aspect 9, wherein the ceramic portion of the connecting portion comprises at least one metallic lead travelling through said ceramic portion for transferring electrical energy or information from within the connecting portion to an outside of the connecting portion and/or from the outside of the connecting portion to an inside of the connecting portion.
11. The implantable medical device according to aspect 9 or 10, wherein the at least one metallic lead is integrated in, or brazed to, the ceramic portion of the connecting portion, such that the at least one metallic lead can pass said ceramic portion without being further insulated.
12. The implantable medical device according to any one of the preceding aspects, wherein
   the first portion comprises a first wireless energy receiver for receiving energy transmitted wirelessly by an external wireless energy transmitter, and an internal wireless energy transmitter configured to transmit energy wirelessly to the second portion, and
   the second portion comprises a second wireless energy receiver configured to receive energy transmitted wirelessly by the internal wireless energy transmitter.
13. The implantable medical device according to aspect 12, wherein the first portion comprises a first energy storage unit connected to the first wireless energy receiver.
14. The implantable medical device according to any one of aspects 12 and 13, wherein the second portion comprises a second energy storage unit connected to the second wireless energy receiver.
15. The implantable medical device according to aspect 14, wherein at least one of the first and second energy storage unit is a solid-state battery.
16. The implantable medical device according to aspect 15, wherein the solid-state battery is a thionyl-chloride battery.
17. The implantable medical device according to any one of aspects 14-16, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
18. The implantable medical device according to any one of aspects 12-17, wherein the first portion comprises a first controller comprising at least one processing unit.
19. The implantable medical device according to any one of aspects 12-18, wherein the second portion comprises a second controller comprising at least one processing unit.
20. The implantable medical device according to any one of aspects 18 and 19, wherein at least one of the first and second controller is connected to a wireless transceiver for communicating wirelessly with an external device.
21. The implantable medical device according to any one of aspects 18 and 19, wherein: the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
22. The implantable medical device according to aspect 21, wherein the second controller is connected to the second wireless communication receiver for receiving wireless communication from the first portion.
23. The implantable medical device according to any one of aspects 12-22, wherein the first wireless energy receiver comprises a first coil and the wireless energy transmitter comprises a second coil.
24. The implantable medical device according to any one of aspects 12-23, wherein the first portion comprises a combined coil, wherein the combined coil is configured to receive energy wirelessly from an external wireless energy transmitter, and transmit energy wirelessly to the second wireless receiver of the second portion.
25. The implantable medical device according to any one of aspects 23 and 24, wherein at least one of the coils are embedded in a ceramic material.
26. The implantable medical device according to any one of the preceding aspects, wherein the first portion is detachably connected to at least one of the second portion and the connecting portion.
27. The implantable medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
28. The implantable medical device according to any one of the preceding aspects, wherein a connecting interface between the connecting portion and the second portion is excentric with respect to the second portion.
29. The implantable medical device according to any one of the preceding aspects, wherein a connecting interface between the connecting portion and the first portion is excentric with respect to the first portion.
30. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of: adapting the medical device to the curvature of the stomach of the patient, and facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   first direction relative to the first member, for stretching the stomach wall between the first and second portions, and
a main portion, wherein:
the first and second members are connected to the main portion, and wherein at least the second member is pivotally connected to the main portion, such that the second member can be displaced in the first direction relative to the first member by the second member pivoting in relation to the main portion, and
the operation device comprises at least one eccentric rotatable engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first
and second portions, and
a main portion, wherein:
the first and second members are connected to the main portion,
and wherein at least the second member is configured to bend or pivot in relation to the main portion, such that the second member can be displaced in the first direction relative to the first member by the second member bending or pivoting in relation to the main portion,
at least the second member comprises a fluid chamber at least partially enclosed by a flexible wall portion, and
the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   first direction, for stretching the stomach wall between the first and second portions, and a main portion, wherein:
the first and second members are connected to the main portion,
at least the second member is configured to bend or pivot in
relation to the main portion, such that the second member can be displaced in a direction away from the first member by the second member bending or pivoting in relation to the main portion, the operation device comprises a first flexible cable for
pulling on the second member for displacing the second member in the direction away from the first member, and
the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received
from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and be fixated to a structure of the body comprising bone, and
a controller configured to:
receive a signal from the sensor, and
control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a
second higher strength, wherein the transmission comprises:
a first and second pulley,
a flexible element configured to be placed around the first and
second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient and comprising a closed curve,
a first member configured to be fixated to the stomach wall of the patient within the footprint of the second member,
wherein the first member is operably connected to the second member such that the first member can be displaced:
relative to the second member, and
within the footprint of the second member, for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, an operation device configured to:
move at least a portion of the first member in a first direction relative to at least one portion of the second member, for stretching a portion of the stomach wall between the first and second members, and at least one of:
move at least a portion of the operable first member in a second direction relative to at least one portion of the second member, and move an operable third member in a third direction for stretching a portion of the stomach wall between the third and second members, and
   a mechanical switching device configured to switch the operation device: from operating the first member to move in the first direction to operating the first member to move in the second direction, or
from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   47. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 448_Electro_Subcutaneous_Control_Pop-Rivet2_Second-Portion-Reservoir

1. An implantable medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and being configured to connect the first portion to the second portion,
   wherein the second portion comprises or forms a reservoir for holding a fluid;
   the implantable medical device further comprising:
   a sealed container configured to protrude into the reservoir;
   an actuator connected to the sealed container, the actuator being configured to expand or retract the sealed container to change the volume of the sealed container for pumping fluid to or from the reservoir;
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes.
2. The implantable medical device according to aspect 1, wherein the actuator comprises an electric motor.
3. The implantable medical device according to aspect 1 or 2, wherein the actuator is arranged in the connecting portion.
4. The implantable medical device according to any one of the preceding aspects, wherein the actuator is partly or fully arranged inside the sealed container.
5. The implantable medical device according to any one of the preceding aspects, wherein the second portion comprises a port in fluid communication with the reservoir for transferring fluid between the reservoir and an additional implant in the patient.
6. The implantable medical device according to aspect 5, further comprising a conduit connected to the port, the conduit being configured to transfer fluid between the reservoir and the additional implant.
7. The implantable medical device according to any one of the preceding aspects, further comprising an injection port for introducing fluid, the injection port being arranged in the first portion.
8. The implantable medical device according to aspect 7, further comprising an internal conduit connecting the injection port to the reservoir.
9. The implantable medical device according to any one of the preceding aspects, wherein the sealed container is a bellows.
10. The implantable medical device according to aspect 9, wherein the bellows is a metallic bellows.
11. The implantable medical device according to any one of the preceding aspects, wherein at least a portion of the sealed container configured to be in contact with fluid comprises metal.
12. The implantable medical device according to any one of the preceding aspects, wherein the volume of the sealed container can be altered such that the volume of the sealed container is more than 60% of the maximum volume of the reservoir.
13. The implantable medical device according to any one of the preceding aspects, wherein the sealed container comprises at least one flexible portion, and wherein the flexible portion enable at least one of compression and expansion of the sealed container.
14. The implantable medical device according to any one of the preceding aspects, wherein the sealed container comprises at least one elastic portion, and wherein the elastic portion enable at least one of compression and expansion of the sealed container.
18. The implantable medical device according to any one of the preceding aspects, further comprising a first energy storage unit and/or a second energy storage unit for powering the actuator.
19. The implantable medical device according to aspect 18, wherein
   the first portion comprises a first wireless energy receiver for receiving energy transmitted wirelessly by an external wireless energy transmitter, and an internal wireless energy transmitter configured to transmit energy wirelessly to the second portion, and
   the second portion comprises a second wireless energy receiver configured to receive energy transmitted wirelessly by the internal wireless energy transmitter.
21. The implantable medical device according to aspect 19, wherein the first energy storage unit is connected to the first wireless energy receiver.
22. The implantable medical device according to aspect 19 or 20, wherein the second portion comprises the second energy storage unit, wherein the second energy storage unit is connected to the second wireless energy receiver.
23. The implantable medical device according to aspect 21, wherein at least one of the first and second energy storage unit is a solid-state battery.
24. The implantable medical device according to aspect 22, wherein the solid-state battery is a thionyl-chloride battery.
25. The implantable medical device according to any one of aspects 22-24, wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.
26. The implantable medical device according to any one of the preceding aspects, wherein the first portion comprises a first controller comprising at least one processing unit.
27. The implantable medical device according to any one of the preceding aspects, wherein the second portion comprises a second controller comprising at least one processing unit.
28. The implantable medical device according to aspect 26 or 27, wherein the first controller and/or the second controller is configured to control the actuator.
29. The implantable medical device according to any one of aspects 26-28, wherein at least one of the first and second controller is connected to a wireless transceiver for communicating wirelessly with an external device.
30. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
wherein at least one of the first and second member is flexible in a second direction substantially perpendicular to the first direction for at least one of:
adapting the medical device to the curvature of the stomach of the patient, and facilitating insertion of the medical device into the body of the patient.
31. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction relative to the first member, for stretching the stomach wall between the first and second portions, and
a main portion, wherein:
the first and second members are connected to the main portion, and wherein at least the second member is pivotally connected to the main portion, such that the second member can be displaced in the first direction relative to the first member by the second member pivoting in relation to the main portion, and
the operation device comprises at least one eccentric rotatable
engagement member configured to engage at least the second member such that the eccentric rotation of the engagement member causes the second member to pivot in relation to the main portion for displacing the second member relative to the first member for stretching the stomach wall between the first and second portions.
32. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   a hydraulic operation device for operating the second member to displace the second member in a first direction relative to the first member, for stretching the stomach wall between the first and second portions, and
a main portion, wherein:
the first and second members are connected to the main portion, and wherein at least the second member is configured to bend or pivot in relation to the main portion, such that the second member can be displaced in the first direction relative to the first member by the second member bending or pivoting in relation to the main portion,
at least the second member comprises a fluid chamber at least
partially enclosed by a flexible wall portion, and
the flexible wall portion causes the second member to bend or pivot when hydraulic fluid is moved to or from the fluid chamber for stretching the stomach wall between the first and second portions.
33. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to be fixated to a first portion of the stomach wall,
   a second member comprising a second stomach engager portion configured to be fixated to a second portion of the stomach wall,
   an operation device for operating the second member to displace the second member in a first direction, for stretching the stomach wall between the first and second portions, and a main portion, wherein:
the first and second members are connected to the main portion,
at least the second member is configured to bend or pivot in relation to the main portion, such that the second member can be displaced in a direction away from the first member by the second member bending or pivoting in relation to the main portion,
the operation device comprises a first flexible cable for
pulling on the second member for displacing the second member in the direction away from the first member, and
the first flexible cable is fixated to the second member at the distal half of thereof.
34. The implantable medical device according to any one of the preceding aspects, wherein the implantable medical device comprises a medical device for actively stretching a stomach wall of a patient for creating a sensation of satiety, the medical device being implantable and comprising:
   a first member comprising a first stomach engager configured to engage a first portion of the stomach wall,
   a second member comprising a second stomach engager portion configured to engage a second portion of the stomach wall, a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force hydraulically or mechanically from the remote unit to the main portion, for operating the second member.
35. The implantable medical device according to any one of the preceding aspects, wherein the implantable medical device comprises a medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   an acoustic sensor configured to sense at least one sound related to the patient swallowing, and
   a controller configured to:
receive a signal from the acoustic sensor, and
control the operation device on the basis of the signal received from the acoustic sensor.
36. The implantable medical device according to any one of the preceding aspects, wherein the implantable medical device comprises medical device for actively stretching a stomach wall of a patient for creating a sensation of satiety, the medical device being implantable and comprising:
   a first member configured to be fixated to a first portion of the stomach wall,
   a second member configured to be fixated to a second portion of the stomach wall,
   an operation device for operating the second member to displace the second member in a first direction relative to the first member, for stretching the stomach wall between the first and second portion,
   a sensor configured to:
sense at least one parameter related to the patient swallowing, and be fixated to a structure of the body comprising bone, and
a controller configured to:
receive a signal from the sensor, and
control the operation device on the basis of the signal received from the sensor.
37. The implantable medical device according to any one of the preceding aspects, wherein the implantable medical device comprises a medical device for actively stretching a stomach wall of a patient for creating a sensation of satiety, the medical device being implantable and comprising:
   a first member comprising:
a first stomach engager configured to engage a first portion of the stomach wall, and
a second stomach engager portion configured to engage a second portion of the stomach wall,
   a second member comprising:
a first stomach engager configured to engage a third portion of the stomach wall, and
a second stomach engager portion configured to engage a fourth portion of the stomach wall,
a main portion, wherein the first and second members are operably connected to the main portion,
   an operation device comprising an actuator, wherein the operation device is configured to displace the actuator, such that the actuator is configured to:
      in a first state, operate the first member for displacing the first stomach engager of the first member in relation to the second stomach engager of the first member, for stretching the stomach wall, and
      in a second state, operate the second member for displacing the first stomach engager of the second member in relation to the second stomach engager of the second member, for stretching the stomach wall.
38. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a flexible shaft for transferring the mechanical force to a body engaging portion, and
   a pre-tensioning device for creating a pre-tension in the flexible shaft.
39. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming a mechanical force created by electrical transforming device from a force having a first speed and a first strength to a force having a second lower speed and a second higher strength, wherein the transmission comprises: a first and second pulley,
a flexible element configured to be placed around the first and second pulley, wherein the flexible element is configured to be pulled by the force having the first speed and first strength, causing the first and second pulley to displace in relation to each other with the force having the second lower speed and the second higher strength, thereby creating the transmission.
40. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient and comprising a closed curve,
a first member configured to be fixated to the stomach wall of the patient within the footprint of the second member,
wherein the first member is operably connected to the second member such that the first member can be displaced: relative to the second member, and
within the footprint of the second member,
for stretching a portion of the stomach wall of the patient between the second and first member.
41. The implantable medical device according to any one of the preceding aspects, wherein the implantable medical device comprises a medical device for actively stretching a stomach wall of a patient for creating a sensation of satiety, the medical device being implantable and comprising: a second member configured to be fixated to the stomach wall of the patient, and
an operable first member connected to the second member and configured to be fixated to the stomach wall at least partially within the footprint of the second member, wherein the first member is configured to be operated to deform such that a first portion of the first member is moved away from a first portion of the second member, for stretching a portion of the stomach wall between the first portion of the first member and the first portion of the second member.
42. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, and a second member configured to be fixated to a second portion of the stomach wall, and an operation device, characterized in that the operation device is configured to pull at least a portion of the first member in a first direction away from at least one portion of the second member, for stretching a portion of the stomach wall between the first portion of the stomach wall and the second portion of the stomach wall for creating the sensation of satiety.
43. The implantable medical device according to any one of the preceding aspects, wherein the implantable medical device comprises a medical device for actively stretching a stomach wall of a patient for creating a sensation of satiety, the medical device being implantable and comprising:
an operable first member configured to be fixated to a first portion of the stomach wall, a second member configured to be fixated to a second portion of the stomach wall, a mechanical operation device configured to move at least a portion of the first member in a first direction away from at least one portion of the second member, for stretching a portion of the stomach wall between the first portion of the stomach wall and the second portion of the stomach wall, for creating the sensation of satiety, wherein a portion of the operation device is placed in a remote unit configured to be placed at a remote location in the body of the patient, and a force transferring element configured to transfer mechanical force from the remote unit to the first member, for operating the first member.
44. The implantable medical device according to any one of the preceding aspects, wherein the implantable medical device comprises a medical device for actively stretching a stomach wall of a patient for creating a sensation of satiety, the medical device being implantable and comprising:
   an operable first member configured to be fixated to a first portion of the stomach wall, a second member configured to be fixated to a second portion of the stomach wall, an operation device configured to:
   move at least a portion of the first member in a first direction relative to at least one portion of the second member, for stretching a portion of the stomach wall between the first and second members, and at least one of:
   move at least a portion of the operable first member in a second direction relative to at least one portion of the second member, and move an operable third member in a third direction for
   stretching a portion of the stomach wall between the third and second members, and
      a mechanical switching device configured to switch the operation device: from operating the first member to move in the first direction to operating the first member to move in the second direction, or from operating the first member to move in the first direction to operating the third member to move in the third direction.
45. The implantable medical device according to any one of the preceding aspects, wherein the implantable medical device comprises an implantable operation device for operating a body engaging portion of a medical implant, the implantable operation device comprising:
   an electrical machine for transforming electrical energy to mechanical force,
   a transmission for transforming rotational force created by the electrical machine to a linear force,
a shaft for transferring rotational and linear force from the operation device to the body engaging portion of the medical implant,
a switch placed between the electrical machine and the shaft, wherein the switch is configured to switch the force exerted on shaft such that:
in a first state, a rotational force is exerted on the shaft, and
in a second, a linear force is exerted in the shaft.
46. The implantable medical device according to any one of the preceding aspects, wherein the implantable medical device comprises:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, characterized by
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
   47. The implantable medical device according to any one of the preceding aspects, wherein the implantable medical device comprises a device for treating obesity of a patient comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   characterized in that the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
48. The implantable medical device according to any one of the preceding aspects, wherein the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   characterized in that the operable stretching device is adapted to be placed against the outside of the stomach wall.
49. The implantable medical device according to any one of the preceding aspects, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   characterized in that the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
50. The implantable medical device according to any one of the preceding aspects, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
      Aspect 454 Dual remote controls
1. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user.
2. The communication system according to aspect 1, wherein the first remote control comprises an input device for receiving a first user input, and wherein the first remote control is configured to transmit the first user input to the implantable medical device.
3. The communication system according to any one of the preceding aspects, wherein the second remote control is configured to receive second user input from the patient display device and to transmit the second user input to the implantable medical device.
4. The communication system according to any one of the preceding aspects, wherein the data comprises a control command for the medical device.
5. The communication system according to any one of the preceding aspects, wherein at least one of the first wireless communication unit and the second wireless communication unit is configured to send or receive data using near-field magnetic induction.
6. The communication system according to aspect 5, wherein at least one of the first wireless communication unit and the second wireless communication unit comprises a transmitter coil for modulating a magnetic field for transmitting the data, and wherein the implantable medical device comprises a receiving coil and an NFMI receiver connected to the receiving coil to receive the data.
7. The communication system according to aspect 6, wherein the transmitter coil is configured to modulate a magnetic field, and the NFMI receiver is adapted to measure the magnetic field in the receiving coil.
8. The communication system according to any one of the preceding aspects, wherein at least one of the first wireless communication unit and the second wireless communication unit is configured to wirelessly charge the medical device using near-field magnetic induction.
9. The communication system according to aspect 9, wherein the medical device comprises a coil for receiving wireless energy for charging the implant via near-field magnetic induction.
10. The communication system according to any one of the preceding aspects, wherein the second and third communication units are configured to transmit and/or receive data using different network protocols.
11. The communication system according to any one of the preceding aspects, wherein the second and third communication units are configured to transmit and/or receive data using different frequency bands.
12. The communication system according to any one of the preceding aspects, wherein at least one of the first remote control, the second remote control and the implantable medical device comprises a Bluetooth transceiver.
13. The communication system according to any one of the preceding aspects, wherein at least one of first remote control, the second remote control and the implantable medical device comprises a UWB transceiver.
14. The communication system according to aspect 10, wherein the network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.
15. The communication system according to any one of the preceding aspects, wherein the second communication unit has a longer effective range than the third communication unit.
16. The communication system according to any one of the preceding aspects, wherein the second remote control is configured to communicate with a consumer electronics device.
17. The communication system according to aspect 16, wherein the patient display device comprises the consumer electronics device.
18. The communication system according to any one of the preceding aspects, wherein the first remote control is configured to control functions of the implantable medical device based on user input to the first remote control.
19. The communication system according to any one of the preceding aspects, wherein the medical device comprises a medical device for actively stretching a stomach wall of a patient for creating a sensation of satiety.
   Aspect 457 Controlling energy transfer Accumulated/PID
1. A method for wireless energy transfer from an external energy source located outside the patient to an internal energy receiver located inside the patient, the internal energy receiver being connected to an implantable medical device for supplying received energy thereto, the method comprising: determining an accumulated amount of received energy over a time period; determining a current change in the received energy;
determining a control signal reflecting the accumulated received energy and the change in the transferred or received energy; controlling the energy transfer based on the control signal.
2. The method according to aspect 1, wherein determining an accumulated amount of received energy is determined by the internal energy receiver.
3. The method according to any one of the preceding aspects, wherein determining a current change is performed by the internal energy receiver.
4. The method according to any one of the preceding aspects, wherein the internal energy received comprises a PID regulator for controlling the energy transfer.
5. The method according to aspect 4, wherein the PID regulator is implemented in a microcontroller.
6. The method according to any one of the preceding aspects, wherein determining a control signal is performed by the internal energy receiver.
7. The method according to aspect 6, wherein the control signal is transmitted to the external energy source, and wherein the external energy source is configured to adjust the transmitted energy based on the control signal.
8. The method according to any one of the preceding aspects, wherein controlling the energy transfer is controlled by the internal energy receiver.
9. The method according to any one of the preceding aspects, wherein controlling the energy transfer is performed by the external energy source.
10. The method according to any one of the preceding aspects, wherein controlling the energy transfer comprises adjusting the energy transfer efficiency.
11. The method according any one of the preceding aspects, wherein the external device comprises a transmitter coil for modulating a magnetic field for transmitting data or transmitting energy, and wherein the implantable medical implant comprises a receiving coil and an NFMI receiver connected to the receiving coil to receive the data or the energy.
12. The method according to any one of the preceding aspects, wherein at least one of the first wireless communication unit and the second wireless communication unit is configured to wirelessly charge the medical implant using near-field magnetic induction.
13. The method according to aspect 9, wherein the medical implant comprises a coil for receiving wireless energy for charging the implant via near-field magnetic induction.
14. The method according to any one of the preceding aspects, further comprising: receiving energy in pulses according to a pulse pattern, and measuring the received pulse pattern.
15. The method according to aspect 15, further comprising: determining that the pulse pattern deviates from a predefined pulse pattern, and controlling the energy transfer based on the determination.
16. The method according to any one of the preceding aspects, further comprising: measuring a temperature in the implantable medical device or in the body of the patient, and controlling the energy transfer in response to the measured temperature.
17. The method according to any one of the preceding aspects, wherein the implantable medical device comprises at least one coil connected to a variable impedance, the method further comprising controlling the energy transfer by controlling the variable impedance.
18. The method according to any one of the preceding aspects, wherein the implantable medical device comprises at least one coil having a plurality of windings, wherein the plurality of windings each are connected to a respective variable impedance, the method further comprising controlling the energy transfer by controlling the respective variable impedance individually.
   Aspect 453 Voice control
1. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant.
2. The method according to aspect 1, wherein adjusting the amplitude comprises at least one of: filtering, cancelling and amplifying the at least one frequency.
3. The method according to any one of the preceding aspects, wherein at least one of the first and second audio training phrase is a spoken audio training phrase. 4. The method according to aspect 3, wherein the spoken audio training phrase is spoken by the patient the implant is implanted in.
5. The method according to any one of the preceding aspects, wherein the first audio training phrase comprises the at least one voice command related to an instruction for the control of the medical implant.
6. The method according to any one of the preceding aspects, wherein the first and second audio training phrases is the same voice command.
7. The method according to any one of the preceding aspects, wherein the first and second audio training phrases are different.
8. The method according to any one of the preceding aspects, wherein creating the transfer function comprises amplifying frequencies muffled by the location of the medical implant in the body of the patient.
9. The method according to any one of the preceding aspects, wherein creating the transfer function comprises filtering or cancelling noise generated by the body.
10. The method according to any one of the preceding aspects, wherein the medical implant is configured to receive voice commands related to an instruction for control of the medical implant.
11. The method according to any one of the preceding aspects, wherein the voice command relates to at least one of:
   performing a function of the medical device;
   using a sensor to measure a parameter relating to a condition of the patient or a condition of the medial implant;
   sending or receiving data from the medical implant.
12. A method of using a voice command to control a medical implant, wherein the method comprises: receiving an audio command phrase for the medical device; applying a transfer function to create an enhanced audio command phrase;
determining a corresponding command for the medical based on the enhance audio command phrase; and
sending the command to the medical device.
13. The method according to aspect 12, further comprising executing, by the medical device, the command.
   Aspect Large coil
1. A system for wirelessly charging an implantable medical implant, when implanted in a body of a patient, the system comprising:
an internal energy receiver comprising a secondary coil, the internal energy receiver being connected to the implantable medical implant;
an external energy transmitter comprising a primary coil for wirelessly transmitting energy to the internal energy receiver via the secondary coil;
wherein a diameter of the primary coil is larger than a diameter of the secondary coil.
2. The system according to any one of the preceding aspects, wherein the system further comprises: an internal controller connected to the internal energy receiver, for controlling the amount of energy received by the internal energy receiver.
3. The system according to any one of the preceding aspects, wherein the internal energy receiver further comprises a measurement unit for measuring a parameter related to the implantable medical implant or the body of the patient.
4. The system according to any one of the preceding aspects, wherein the controller is configured to measure the accumulated energy received by the internal energy receiver over a period of time and to measure a current change in energy received, and to control the energy received based on the accumulated energy and the current change.
5. The system according to any one of the preceding aspects, wherein the controlled comprises a Proportional—Integral—Derivative, PID, regulator for controlling the received energy.
6. The system according to any one of the preceding aspects, wherein the internal energy received comprises a variable impedance.
7. The system according to aspect 6, when depending on any one of aspects 2-5, wherein the internal energy receiver is configured to control the resonant frequency by controlling the variable impedance.
8. The system according to aspect 7, wherein the controller is configured to vary the variable impedance in response to a measured parameter deviating from a predetermined interval or exceeding a threshold value.
9. The system according to aspect 7, wherein the parameter relates to the energy received by the coil over a time period.
10. The system according to aspect 8 or 9, wherein the measurement unit is configured to measure a parameter related to a change in energy received by the coil.
11. The system according to aspect 3-10, wherein the receiving unit is configured to receive transferred energy in pulses according to a pulse pattern, and wherein the measurement unit is configured to measure a parameter related to the pulse pattern.
12. The system according to aspect 3-10, wherein the receiving unit is configured to receive transferred energy in pulses according to a pulse pattern, and wherein the measurement unit is configured to measure a parameter related to the pulse pattern.
13. The system according to aspect 3-11, wherein the controller is configured to control the variable impedance in response to the pulse pattern deviating from a predefined pulse pattern.
14. The system according to aspect 6-13, wherein:
the variable impedance comprises a resistor and a capacitor,
the variable impedance comprises a resistor and an inductor,
the variable impedance comprises an inductor and a capacitor,
the variable impedance comprises a digitally tuned capacitor,
the variable impedance comprises a digital potentiometer, or
the variable impedance comprises a variable inductor.
15. The system according any one of the preceding aspects, wherein the diameter of the primary coil is more than 0.5 cm.
16. The system according to aspect 15, wherein the diameter of the primary coil is more than 10 cm.
17. The system according to aspect 16, wherein the diameter of the primary coil is more than 15 cm.
18. The system according to aspect 17, wherein the diameter of the primary coil is more than 20 cm.
19. The system according to aspect 18, wherein the diameter of the primary coil is more than 30 cm.
20. The system according to aspect 19, wherein the diameter of the primary coil is more than 50 cm.
21. The system according to any one of aspects 1-15, wherein the area of the primary coil is more than 0.5 cm2.
22. The system according to aspect 21, wherein the area of the primary coil is more than 2 cm2.
23. The system according to aspect 22, wherein the area of the primary coil is more than 10 cm2.
24. The system according to aspect 23, wherein the area of the primary coil is more than 100 cm2.
25. The system according to aspect 24, wherein the area of the primary coil is more than 300 cm2.
26. The system according to aspect 25, wherein the area of the primary coil is more than 500 cm2.
27. The system according to aspect 26, wherein the area of the primary coil is more than 800 cm2.
   Aspect 456 NFMI communication and energy transfer
1. A system for communication with an implantable medical device, when implanted in a body of a patient, comprising:
an internal communications unit, connected to or comprised in the implantable medical device; an external communications unit,
wherein the internal communications unit and the external communications unit are configured to communicate using near field magnetic induction.
2. The system according to aspect 1, wherein:
the internal communication unit comprises an internal NFMI receiver and an internal coil connected to the internal NFMI receiver, the internal NFMI receiver being configured to measure an induced voltage in the internal coil,
the external communications unit comprises an external NFMI transmitter and an external coil connected to the external NFMI transmitter, and
the external coil and the external NFMI transmitter are configured to modulate a magnetic field for sending data to the implantable medical device 603 via the internal coil.
3. The system according to aspect 2, wherein the external NFMI transmitter further comprises a capacitor for tuning the external coil and the external NFMI transmitter.
4. The system according to aspect 3, wherein the internal NFMI receiver comprises a tunable resistor and capacitor tank for turning the internal coil and the internal NFMI receiver.
5. The system according to any one of the preceding aspects, wherein:
the internal communication unit comprises an internal NFMI transmitter and an internal coil connected to the internal NFMI transmitter,
the external communications unit comprises an external NFMI receiver and an external coil connected to
the external NFMI receiver, the external NFMI receiver being configured to measure an induced voltage in the external coil,
the internal coil and the internal NFMI transmitter are configured to modulate a magnetic field for sending data to the external communications unit via the external coil.
6. The system according to aspect 5, wherein the internal NFMI transmitter further comprises a capacitor for tuning the internal coil and the internal NFMI receiver.
7. The system according to aspect 5 or 6, wherein the external NFMI receiver comprises a tunable resistor and capacitor tank for turning the external NFMI receiver and the external coil.
8. The system according to any one of the preceding aspects, wherein the implantable medical device comprises an active portion configured to monitor, treat or perform a function of a body of a patient.
9. The system according to aspect 8, wherein the active portion is not a pacemaker, hearing aid or a neurostimulation implant.
10. The system according to any one of the preceding aspects, wherein the internal communications unit is adapted to be implanted at a tissue depth of at least 8 cm or at least 15 cm.
11. The system according to any one of the preceding aspects, wherein the internal communications unit is adapted to be implanted in an abdomen of a patient.
12. The system according to any one of the preceding aspects, wherein the external communications unit is configured to communicate with another external device.
13. The system according to any one of the preceding aspects, wherein the internal communications unit is configured to encrypt data before transmitting it to the external communications unit.
14. The system according to aspect 13, wherein the external communications unit is configured to relay the encrypted data to the another external device without decrypting it.
15. The communication system according to any one of the preceding aspects, wherein the medical device comprises a medical device for actively stretching a stomach wall of a patient for creating a sensation of satiety.
   Aspect 459 Resonant circuit
1. An implantable medical device adapted to receive transcutaneously and wirelessly transmitted energy, the implantable medical device comprising:
an energy consuming part, a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and a controller configured to control the subcutaneously received energy based on the parameter by controlling the first or the second impedance unit.
2. The implantable medical device according to aspect 1, wherein the first energy receiving unit has a first resonant frequency based on the inductance of the first coil and the impedance of the first impedance unit, and the second energy receiving unit has a second resonant frequency based on the inductance of the second coil and the impedance of second impedance unit.
3. The implantable medical device according to aspect 2, wherein the first receiving unit has a resonant frequency different from the resonant frequency of the second receiving unit.
4. The implantable medical device according to any preceding aspect, wherein the first and second impedance units are connected in parallel to the respective coil.
5. An implantable medical device adapted to receive transcutaneously and wirelessly transmitted energy, the implantable medical device comprising:
an energy consuming part,
   a receiving unit configured for receiving transcutaneously transferred energy and transferring the received energy to the energy consuming part, comprising:
a first coil portion and a second coil portion, and
a first impedance unit and a second impedance unit, wherein the first impedance unit is connected to the first coil portion and the second impedance unit is connected to the second coil portion, a measurement unit configured to measure a parameter related to energy transfer, and
a controller configured to control the subcutaneously received energy based on the parameter by controlling the first or the second impedance unit.
6. The implantable medical device according to aspect 5, wherein the first coil portion and the second coil portion are at least one of: portions of the same coil, or portions or different coils connected in series.
7. The implantable medical device according to aspect 5 or 6, wherein the first coil portion and the second coil portion have the same inductance, or the first coil portion has a different inductance than the second coil portion.
8. The implantable medical device according to any one of aspects 5-7, wherein the first impedance is connected in parallel to the first coil portion and the second impedance is connected in parallel to the second coil portion.
9. The implantable medical device according to any one of aspects 5-8, wherein one of the first coil portion and the second coil portion are overlapping the other of the first coil portion and the second coil portion, or the first coil portion and the second coil portion are not overlapping with the other of the first coil portion and the second coil portion.
10. The implantable medical device according to any one of aspects 5-9, wherein the first coil portion and the first impedance unit has a first resonance frequency, and the second coil portion and the second impedance unit has a second resonance frequency.
11. The implantable medical device according to aspect 10, wherein the first resonance frequency is different from the second resonance frequency.
12. The implantable medical device according to any preceding aspect, wherein the first or second impedance unit is a capacitor.
13. The implantable medical device according to any preceding aspect, wherein the first impedance unit and the second impedance unit have different impedances.
14. An implantable medical device comprising:
an energy consuming part,
a first receiving unit comprising:
a first coil configured for receiving transcutaneously transferred energy and transferring the received energy to the energy consuming part,
a first impedance electrically connected to the coil,
a second receiving unit comprising:
a second coil portion and a third coil portion configured for receiving transcutaneously transferred energy and transferring the received energy to the energy consuming part, and
a second impedance unit and a third impedance unit, wherein the second impedance unit is connected to the second coil portion and the third impedance unit is connected to the third coil portion,
a measurement unit configured to measure a parameter related to energy transfer, and
a controller configured to control the subcutaneously received energy based on the parameter by controlling the first, the second or the third impedance unit.
15. The communication system according to any one of the preceding aspects, wherein the medical device comprises a medical device for actively stretching a stomach wall of a patient for creating a sensation of satiety.

1. A medical device for actively stretching a stomach wall of a patient for creating a sensation of satiety, the medical device being implantable and comprising:
a first member comprising a first stomach engager configured to engage a first portion of the stomach wall,
a second member comprising a second stomach engager portion configured to engage a second portion of the stomach wall,
a main portion, wherein the first and second members are connected to the main portion,
an operation device for operating at least the second member to displace the second member in a direction away from the first member, for stretching the stomach wall between the first and second portions, wherein a portion of the operation device is placed in a remote unit configured to be placed at a remote location in the body of the patient, and
a force transferring element configured to transfer force mechanically from the remote unit to the main portion, for operating the second member.

2. The medical device according to claim 1, wherein the portion of the operation device placed in the remote unit comprises at least one electrical machine.

3. The implantable operation device according to claim 2, wherein the electrical machine comprises a solenoid.

4. The implantable operation device according to claim 2, wherein the electrical machine comprises an electrical motor.

5. The medical device according to claim 4, wherein the portion of the operation device placed in the remote unit further comprises a gear system configured to reduce the velocity and increase the force of the movement generated by the electrical motor.

6. The medical device according to claim 1, wherein a portion of the operation device is placed in the main portion, and wherein the portion of the operation device placed in the main portion comprises a receiving portion configured to receive mechanical force, and
a transmission for transforming the received mechanical force into a force for displacing at least one of the members for stretching the stomach wall.

7. The medical device according to claim 6, wherein the transmission comprises a gear system configured to reduce the velocity and increase the force of the received mechanical force.

8. The medical device according to claim 6, wherein the receiving portion is configured to receive a rotating mechanical force, and wherein the transmission is configured to transform the received rotating mechanical force into a liner mechanical force.

9. The medical device according to claim 6, wherein the receiving portion is configured to receive a linear mechanical force.

10. The medical device according to claim 1, wherein the remote unit comprises a magnetic coupling for transferring mechanical force through the housing.

11. The medical device according to claim 1, wherein the force transferring element has a length of more than 5 cm or more than 10 cm.

12. The medical device according to claim 1, wherein the remote unit comprises:
a first portion configured to be placed on a first side of the tissue portion, the first portion having a first cross-sectional area in a first plane and comprises a first surface configured to engage a first tissue surface of the tissue portion,
a second portion configured to be placed on a second side of the tissue portion, the second portion having a second cross-sectional area in a second plane and comprises a second surface configured to engage a second tissue surface of the tissue portion, and
a connecting portion configured to be placed through a hole in the tissue portion, the connecting portion having a third cross-sectional area in a third plane and is configured to connect the first portion to the second portion, wherein:
the first, second and third planes are parallel to each other,
the third cross-sectional area is smaller than the first and second cross-sectional areas, such that the first and second portions are prevented from travelling through the hole in the tissue portion in a direction perpendicular to the first, second and third planes.

13. The medical device according to claim 12, wherein the first portion comprises a first wireless energy receiver for receiving energy transmitted wirelessly from an external wireless energy transmitter, and an internal wireless energy transmitter configured to transmit energy wirelessly to the second portion, and the second portion comprises a second wireless energy receiver for receiving energy from the first portion wirelessly.

14. The medical device according to claim 12, wherein the first portion is detachably connected to at least one of the second portion and the connecting portion.

15. The medical device according to claim 12, wherein the connecting portion comprising a portion having a flange area being larger than the third cross-sectional area, such that the portion is hindered from travelling through the hole in the tissue portion, such that the second portion and the connecting portion can be held in position by the tissue portion of the patient also when the first portion is disconnected from the connecting portion.

16. The medical device according to claim 1, wherein the operation device is configured to displace the first member in a direction away from the second member, for stretching the stomach wall between the first and second portions.

17. The medical device according to claim 1, wherein the force transferring element configured to transfer force mechanically from the remote unit to the main portion comprises a flexible shaft configured to transmit rotary motion.

18. The medical device according to claim 17, wherein the flexible shaft comprises at least one of a spring and an elastic material.

19. The medical device according to claim 1, wherein the force transferring element configured to transfer force mechanically from the remote unit to the main portion comprises a flexible shaft configured to transmit linear motion.

20. The medical device according to claim 19, wherein the flexible shaft comprises at least one of a spring and an elastic material.
